(19) United States
(12) Patent Application Publication
(10) Pub. No.: US 2009/0280153 A1
(43) Pub. Date: Nov. 12, 2009

(54) electrical devices, anti-scarring agents, and therapeutic compositions

(75) Inventors: William L. Hunter, Vancouver (CA); Philip M. Toleikis, Vancouver (CA); David M. Gravett, West Vancouver (CA); Arpita Maiti, Vancouver (CA); Richard T. Liggins, Coquitlam (CA); Aniko Takacs-Cox, North Vancouver (CA); Rui Avelar, Vancouver (CA); Pierre E. Signore, Vancouver (CA); Troy A. E. Loss, North Vancouver (CA); Anne Hutchinson, Burnaby (CA); Gaye McDonald-Jones, North Delta (CA); Fara Lakhani, Vancouver (CA)

Correspondence Address: SEED INTELLECTUAL PROPERTY LAW GROUP PLLC, 701 FIFTH AVENUE, SUITE 5400, SEATTLE, WA, 98104-7092, US

(73) Assignee: Angiotech International AG, Zug (CH)

(21) Appl. No.: 11/919,274

(22) PCT Filed: Mar. 31, 2006
(86) PCT No.: PCT/US06/11610

§ 371 Date: Jul. 9, 2009

Related U.S. Application Data
(60) Provisional application No. 60/679,293, filed on May 10, 2005.
(60) Provisional application No. 60/679,292, filed on May 10, 2005.

(51) Int. Cl. A61F 2/00; A61K 31/335; A61N 1/00

(52) U.S. Cl. 424/423; 514/450; 607/2

(57) ABSTRACT

Electrical devices (e.g., cardiac rhythm management and neurostimulation devices) for contact with tissue are used in combination with an anti-scarring agent in order to inhibit scarring that may otherwise occur when the devices are implanted within an animal.

### BACKGROUND OF THE INVENTION

1. Field of the Invention

The present invention relates generally to pharmaceutical compositions and electrical devices that inhibit fiborisis or gliosis and methods for making and using such compositions and electrical devices.

2. Description of the Related Art

Medical devices having electrical components, such as electrical pacing or stimulating devices, can be implanted in the body to provide electrical conduction to the central and peripheral nervous system (including the autonomic system), cardiac muscle tissue (including myocardial conduction pathways), smooth muscle tissue and skeletal muscle tissue. These electrical impulses are used to treat many bodily dysfunctions and disorders by blocking, masking, stimulating, or replacing-electrical signals within the body. Examples include pacemaker leads used to maintain the normal rhythmic beating of the heart; defibrillator leads used to “re-start” the heart when it stops beating; peripheral nerve stimulating devices to treat chronic pain; deep brain electrical stimulation to treat conditions such as tremor, Parkinson's disease, movement disorders, epilepsy, depression and psychiatric disorders; and vagal nerve stimulation to treat epilepsy, depression, anxiety, obesity, migraine and Alzheimer's Disease.

The clinical function of an electrical device such as a cardiac pacemaker lead, neurostimulation lead, or other electrical lead depends upon the device being able to effectively maintain intimate anatomical contact with the target tissue (typically electrically excitable cells such as muscle or nerve) such that electrical conduction from the device to the tissue can occur. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue (or glial tissue—called “gliosis”—when it occurs within the central nervous system). Scarring (i.e., fibrosis or gliosis) can also result from trauma to the anatomical structures and tissue surrounding the implant during the implantation of the device. Lastly, fibrous encapsulation of the device can occur even after a successful implantation if the device is manipulated (some patients continuously “fiddle” with a subcutaneous implant) or irritated by the daily activities of the patient. When scarring occurs around the implanted device, the electrical characteristics of the electrode-tissue interface degrade, and the device may fail to function properly. For example, it may require additional electrical current from the lead to overcome the extra resistance imposed by the intervening scar (or glial) tissue. This can shorten the battery life of an implant (making more frequent removal and re-implantation necessary), prevent electrical conduction altogether (rendering the implant clinically ineffective) and/or cause damage to the target tissue. Additionally, the surrounding tissue may be inadvertently damaged from the inflammatory foreign body response, which can result in loss of function or tissue necrosis.

### BRIEF SUMMARY OF THE INVENTION

Briefly stated, the present invention discloses pharmaceutical agents that inhibit one or more aspects of the production of excessive fibrous (scar) or glial tissue. In one aspect, the present invention provides compositions for delivery of selected therapeutic agents via medical implants or implantable electrical medical devices, as well as methods for making and using these implants and devices. Compositions and methods are described for coating electrical medical devices and implants with drug-delivery compositions such that the pharmaceutical agent is delivered in therapeutic levels over a period sufficient to prevent the device electrode from being encapsulated in fibrous or glial tissue and to allow normal electrical conduction to occur. Alternatively, locally administered compositions (e.g., topicals, injectables, liquids, gels, sprays, microspheres, pastes, wafers) containing an inhibitor of fibrosis (or gliosis) are described that can be applied to the tissue adjacent to the electrical medical device or implant, such that the pharmaceutical agent is delivered in therapeutic levels over a period sufficient to prevent the device electrode from being encapsulated in fibrous or glial tissue. And finally, numerous specific cardiac and neurological implants and devices are described that produce superior clinical results as a result of being coated with agents that reduce excessive scarring and fibrous (or glial) tissue accumulation as well as other related advantages.

Within one aspect of the invention, drug-coated or drug-impregnated implants and medical devices are provided which reduce fibrosis or gliosis in the tissue surrounding the electrical device or implant, or inhibit scar development on the device/implant surface (particularly the electrical lead), thus enhancing the efficacy of the procedure. For example, it may require additional electrical current from the lead to overcome the extra resistance imposed by the intervening scar (or glial) tissue. This can shorten the battery life of an implant (making more frequent removal and re-implantation necessary), prevent electrical conduction altogether (rendering the implant clinically ineffective) and/or cause damage to the target tissue. Within various embodiments, fibrosis or gliosis is inhibited by local or systemic release of specific pharmacological agents that become localized to the adjacent tissue.

The repair of tissues following a mechanical or surgical intervention, such as the implantation of an electrical device, involves two distinct processes: (1) regeneration (the replacement of injured cells by cells of the same type and (2) fibrosis (the replacement of injured cells by connective tissue). There are four general components to the process of fibrosis (or scarring) including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). As utilized herein, “inhibits (reduces) fibrosis” should be understood to refer to agents or compositions which decrease or limit the formation of fibrous tissue (i.e., by reducing or inhibiting one or more of the processes of angiogenesis, connective tissue cell migration or proliferation, ECM production, and/or remodeling). In addition, numerous therapeutic agents described in this invention may have the additional benefit of also reducing tissue regeneration where appropriate.

It should be noted that in implantation procedures that cause injuries to the central nervous system (CNS), fibrosis is replaced by a process called gliosis (the replacement of injured or dead cells with glial tissue). Glial cells form the supporting tissue of the CNS and are comprised of macroglia (astrocytes, oligodendrocytes, ependyma cells) and microglia cells. Of these cell types, astrocytes are the principle cells responsible for repair and scar formation in the brain and spinal cord. Gliosis is the most important indicator of CNS damage and consists of astrocyte hypertrophy (increase in size) and hyperplasia (increase in cell number as a result of cell division) in response to injury or trauma, such as that caused by the implantation of a medical device. Astrocytes are responsible for phagocytosing dead or damaged tissue and repairing the injury with glial tissue and thus, serve a similar role to that performed by fibroblasts in scarring outside the brain. In medical devices implanted into the CNS, it is the hypertrophy and proliferation of astrocytes (gliosis) that leads to the formation of a “scar-like” capsule around the implant which can interfere with electrical conduction from the device to the neuronal tissue.

Within certain embodiments of the invention, an implant or device is adapted to release an agent that inhibits fibrosis or gliosis through one or more of the mechanisms sited herein. Within certain other embodiments of the invention, an implant or device contains an agent that while remaining associated with the implant or device, inhibits fibrosis between the implant or device and the tissue where the implant or device is placed by direct contact between the agent and the tissue surrounding the implant or device.

Within related aspects of the present invention, cardiac and neurostimulation devices are provided comprising an implant or device, wherein the implant or device releases an agent which inhibits fibrosis (or gliosis) in vivo. Within yet other aspects of the present invention, methods are provided for manufacturing a medical device or implant, comprising the step of coating (e.g., spraying, dipping, wrapping, or administering drug through) a medical device or implant. Additionally, the implant or medical device can be constructed so that the device itself is comprised of materials which inhibit fibrosis in or around the implant. A wide variety of electrical medical devices and implants may be utilized within the context of the present invention, depending on the site and nature of treatment desired.

Within various embodiments of the invention, the implant or device is further coated with a composition or compound, which delays the onset of activity of the fibrosis-inhibiting (or gliosis-inhibiting) agent for a period of time after implantation. Representative examples of such agents include heparin, PLGA/MePEG, PLA, and polyethylene glycol. Within further embodiments, the fibrosis-inhibiting (or gliosis-inhibiting) implant or device is activated before, during, or after deployment (e.g., an inactive agent on the device is first activated to one that reduces or inhibits an in vivo fibrotic or gliotic reaction).

Within various embodiments of the invention, the tissue surrounding the implant or device is treated with a composition or compound that contains an inhibitor of fibrosis or gliosis. Locally administered compositions (e.g., topicals, injectables, liquids, gels, sprays, microspheres, pastes, wafers) or compounds containing an inhibitor of fibrosis (or gliosis) are described that can be applied to the surface of, or infiltrated into, the tissue adjacent to the electrical medical device or implant, such that the pharmaceutical agent is delivered in therapeutic levels over a period sufficient to prevent the device electrode from being encapsulated in fibrous or glial tissue. This can be done in lieu of coating the device or implant with a fibrosis/gliosis-inhibitor, or done in addition to coating the device or implant with a fibrosis/gliosis-inhibitor. The local administration of the fibrosis/gliosis-inhibiting agent can occur prior to, during, or after implantation of the electrical device itself.

Within various embodiments of the invention, an electrical device or implant is coated on one aspect, portion or surface with a composition which inhibits fibrosis, as well as being coated with a composition or compound which promotes scarring on another aspect, portion or surface of the device (i.e., to affix the body of the device into a particular anatomical space). Representative examples of agents that promote fibrosis and scarring include silk, silica, crystalline silicates, bleomycin, quartz dust, neomycin, talc, metallic beryllium and oxides thereof, retinoic acid compounds, copper, leptin, growth factors, a component of extracellular matrix; fibronectin, collagen, fibrin, or fibrinogen, polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGFβ, PDGF, VEGF, bFGF, TNFα, NGF, GM-CSF, IGF-1, IL-1, IL-1-β, IL-8, IL-6, and growth hormone); connective tissue growth factor (CTGF) as well as analogues and derivatives thereof.

Also provided by the present invention are methods for treating patients undergoing surgical, endoscopic or minimally invasive therapies where an electrical device or implant is placed as part of the procedure. As utilized herein, it should be understood that “inhibits fibrosis or gliosis” refers to a statistically significant decrease in the amount of scar tissue in or around the device or an improvement in the interface between the electrical device or implant and the tissue, which may or may not lead to a permanent prohibition of any complications or failures of the device/implant.

The pharmaceutical agents and compositions are utilized to create novel drug-coated implants and medical devices that reduce the foreign body response to implantation and limit the growth of reactive tissue on the surface of, or around in the tissue surrounding the device, such that performance is enhanced. Electrical medical devices and implants coated with selected pharmaceutical agents designed to prevent scar tissue overgrowth and improve electrical conduction can offer significant clinical advantages over uncoated devices.

For example, in one aspect the present invention is directed to electrical stimulatory devices that comprise a medical implant and at least one of (i) an anti-scarring agent and (ii) a composition that comprises an anti-scarring agent. The agent is present so as to inhibit scarring that may otherwise occur when the implant is placed within an animal. In another aspect the present invention is directed to methods wherein both an implant and at least one of (i) an anti-scarring agent and (ii) a composition that comprises an anti-scarring agent, are placed into an animal, and the agent inhibits scarring that may otherwise occur. These and other aspects of the invention are summarized below.

Thus, in various independent aspects, the present invention provides a device, comprising a cardiac or neurostimulator implant and an anti-scarring agent or a composition comprising an anti-scarring agent, wherein the agent inhibits scarring. These and other devices are described in more detail herein.

In additional aspects, for each of the aforementioned devices combined with each of the agents described herein, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In addition to devices, the present invention also provides methods. For example, in additional aspects of the present invention, for each of the aforementioned devices, and for each of the aforementioned combinations of the devices with the anti-scarring (or anti-gliotic) agents, the present invention provides methods whereby a specified device is implanted into an animal, and a specified agent associated with the device inhibits scarring (or gliosis) that may otherwise occur. Each of the devices identified herein may be a “specified device”, and each of the anti-scarring agents identified herein may be an “anti-scarring agent”, where the present invention provides, in independent embodiments, for each possible combination of the device and the agent.

The agent may be associated with the device prior to the device being placed within the animal. For example, the agent (or composition comprising the agent) may be coated onto an implant, and the resulting device then placed within the animal. In addition, or alternatively, the agent may be independently placed within the animal in the vicinity of where the device is to be, or is being, placed within the animal. For example, the agent may be sprayed or otherwise placed onto, adjacent to, and/or within the tissue that will be contacting the medical implant or may otherwise undergo scarring. To this end, the present invention provides placing a cardiac or neurostimulation implant and an anti-scarring (or anti-gliosis) agent or a composition comprising an anti-scarring (or anti-gliosis) agent into an animal host, wherein the agent inhibits scarring or gliosis.

In additional aspects, for each of the aforementioned methods used in combination with each of the agents described herein, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In each of the aforementioned devices, compositions, methods of making the aforementioned devices or compositions, and methods of using the aforementioned devices or compostions, the present invention provides that the anti-fibrotic agent may be one or more of the following: 1) an anti-fibrotic agent that inhibits cell regeneration, 2) an anti-fibrotic agent that inhibits angiogenesis, 3) an anti-fibrotic agent that inhibits fibroblast migration, 4) an anti-fibrotic agent that inhibits fibroblast proliferation, 5) an anti-fibrotic agent that inhibits deposition of extracellular matrix, 6) an anti-fibrotic agent inhibits tissue remodeling, 7) an adensosine A2A receptor antagonist, 8) an AKT inhibitor, 9) an alpha 2 integrin antagonist, wherein the alpha 2 integrin antagonist is Pharmaprojects No. 5754 (Merck KgaA), 10) an alpha 4 integrin antagonist, 11) an alpha 7 nicotinic receptor agonist, 12) an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof, 13) an apoptosis antagonist, 14) an apoptosis activator, 15) a beta 1 integrin antagonist, 16) a beta tubulin inhibitor, 17) a blocker of enzyme production in Hepatitis C, 18) a Bruton's tyrosine kinase inhibitor, 19) a calcineurin inhibitor, 20) a caspase 3 inhibitor, 21) a CC chemokine receptor antagonist, 22) a cell cycle inhibitor selected from the group consisting of SNS-595 (Sunesis), synthadotin, KRX-0403, homoharringtonine, and an analogue or derivative thereof, 23) a cathepsin B inhibitor, 24) a cathepsin K inhibitor, wherein the cathepsin K inhibitor is 462795 (GlaxoSmithKline), INPL-022-D6 (Amura Therapeutics), or an analogue or derivative thereof, 25) a cathepsin L inhibitor, 26) a CD40 antagonist, 27) a chemokine receptor agonist, 28) a chymase inhibitor, 29) a collagenase antagonist, 30) a CXCR antagonist, 31) a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), CGP 74514A, bohemine, olomoucine (CAS No. 101622-51-9), indole-3-carbinol (CAS No. 700-06-1), and an analogue or derivative thereof, 32) a cyclooxygenase 1 inhibitor, 33) a DHFR inhibitor, 34) a dual integrin inhibitor, 35) an elastase inhibitor, 36) an elongation factor-1alpha inhibitor, 37) an endothelial growth factor antagonist, 38) an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), lavendustin A (CAS No. 125697-92-9), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), SU 1498 (a VEGF-R inhibitor), a VEGFR-2 kinase inhibitor (Bristol-Myers Squibb), XL-647 (Exelixis), a KDR inhibitor from Abbott Laboratories, sorafenib tosylate, and an analogue or derivative thereof, 39) an endotoxin antagonist, 40) an epothilone and tubulin binder, 41) an estrogen receptor antagonist, 42) an FGF inhibitor, 43) a farnexyl transferase inhibitor, 44) a farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof, 45) an FLT-3 kinase inhibitor, 46a) an FGF receptor kinase inhibitor, 47) a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), mevastatin, and an analogue or derivative thereof, 48) a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), radicicol from Humicola fuscoatra (CAS No. 12772-57-5), and an analogue or derivative thereof, 49) a histone deacetylase inhibitor, 50) an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), cerivastatin Na (CAS No. 143201-11-0), and an analogue or derivative thereof, 51) an ICAM inhibitor, 52) an IL, ICE and IRAK antagonist, wherein the antagonist is a CJ-14877, CP-424174 (Pfizer), NF-61 (Negma-Lerads), and an analogue or derivative thereof, 53) an IL-2 inhibitor, 54) an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof, 55) an IMPDH (inosine monophosphate), 56) an integrin antagonist, 57) an interleukin antagonist, 58) an inhibitor of type III receptor tyrosine kinase, 59) an irreversible inhibitor of enzyme methionine aminopeptidase type 2, 60) an isozyme selective delta protein kinase C inhibitor, 61) a JAK3 enzyme inhibitor, 62) a JNK inhibitor, 63) a kinase inhibitor, 64) a kinesin antagonist, 65) a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof, 66) a MAP kinase inhibitor, 67) a matrix metalloproteinase inhibitor, 68) an MCP-CCR2 inhibitor, 69) an mTOR inhibitor, 70) an mTOR kinase inhibitor,71) a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, dolastatin 15 (CAS No. 123884-00-4), vincamine, and an analogue or derivative thereof, 72) an MIF inhibitor, 73) an MMP inhibitor, 74) a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-O₂-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof, 75) an NF kappa B inhibitor selected from the group consisting of emodin (CAS No. 518-82-1), AVE-0545 or AVE-0547 (Sanofi-Aventis), bortezomib (CAS No. 179324-69-7) (Millennium Pharmaceuticals), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), Bay 11-7085, and an analogue or derivative thereof, 76) a nitric oxide agonist, 77) an ornithine decarboxylase inhibitor, 78) a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), SKF86002 (CAS No. 72873-74-6), RPR-200765A (Sanofi-Aventis), SD-282 (Johnson & Johnson), TAK-715 (Takeda), and an analogue or derivative thereof, 79) a palmitoyl-protein thioesterase inhibitor, 80) a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof, 81) a peroxisome proliferators-activated receptor agonist selected from the group consisting of (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), LBM642, WY-14,643 (CAS No. 50892-23-4), GW7647, fenofibric acid (CAS No. 42017-89-0), MCC-555 (CAS No. 161600-01-7), GW9662, GW1929, GW501516, L-165,041 (CAS No. 79558-09-1), and an analogue or derivative thereof, 82) a phosphatase inhibitor, 83) a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), theobromine (CAS No. 83-67-0), papverine hydrochloride (CAS No. 61-25-6), quercetin dehydrate (CAS No. 6151-25-3), YM 976 (CAS No. 191219-80-4), irsogladine (CAS No. 57381-26-7), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof, 84) a PKC inhibitor, 85) a platelet activating factor antagonist, 86) a platelet-derived growth factor receptor kinase inhibitor, 87) a prolyl hydroxylase inhibitor, 88) a polymorphonuclear neutrophil inhibitor, 89) a protein kinase B inhibitor, 90) a protein kinase C stimulant, 91) a purine nucleoside analogue, 92) a purinoreceptor P2X antagonist, 93) a Raf kinase inhibitor, 94) a reversible inhibitor of ErbB1 and ErbB2, 95) a ribonucleoside triphosphate reductase inhibitor, 96) an SDF-1 antagonist, 97) a sheddase inhibitor, 98) an SRC inhibitor, 99) a stromelysin inhibitor, 100) an Syk kinase inhibitor, 101) a telomerase inhibitor, 102) a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-1 antagonists from Sydney, non-industrial source), TGF-βI receptor kinase inhibitors from Eli Lilly, TGF-β receptor inhibitors from Johnson & Johnson, and an analogue or derivative thereof, 103) a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof, 104) a tumor necrosis factor antagonist, 105) a Toll receptor inhibitor, 106) a tubulin antagonist, 107) a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), herbimycin A, and an analogue or derivative thereof, 108) a VEGF inhibitor, 109) a vitamin D receptor agonist, 110) ZD-6474 (an angiogenesis inhibitor), 111) AP-23573 (an mTOR inhibitor), 112) synthadotin (a tubulin antagonist), 113) S-0885 (a coliagenase inhibitor), 114) aplidine (an elongation factor-1 alpha inhibitor), 115) ixabepilone (an epithilone), 116) IDN-5390 (an angiogenesis inhibitor and an FGF inhibitor), 117) SB-2723005 (an angiogenesis inhibitor), 118) ABT-518 (an angiogenesis inhibitor), 119) combretastatin (an angiogenesis inhibitor), 120) anecortave acetate (an angiogenesis inhibitor), 121) SB-715992 (a kinesin antagonist), 122) temsirolimus (an mTOR inhibitor), and 123) adalimumab (a TNFα antagonist), 124) erucylphosphocholine (an ATK inhibitor), 125) alphastatin (an angiogenesis inhibitor), 126) bortezomib (an NF Kappa B inhibitor), 127) etanercept (a TNFα antagonist and TACE inhibitor), 128) humicade (a TNFα inhibitor), and 129) gefitinib (a tyrosine kinase inhibitor), 130) a histamine receptor antagonist selected from the group consisting of phenothiazines (e.g., promethazine), alkylamines (e.g., chlorpheniramine (CAS No. 7054-11-7), brompheniramine (CAS No. 980-71-2), fexofenadine hydrochloride, promethazine hydrochloride, loratadine, ketotifen fumarate salt, and acrivastine), methylxanthines (e.g., theophylline, theobromine, and caffeine), cimetidine (available under the tradename TAGAMET from SmithKline Beecham Phamaceutical Co., Wilmington, Del.), ranitidine (available under the tradename ZANTAC from Warner Lambert Company, Morris Plains, N.J.), famotidine (available under the tradename PEPCID from Merck & Co., Whitehouse Station, N.J.), nizatidine (available under the tradename AXID from Reliant Pharmaceuticals, Inc., Liberty Corner, N.J.), nizatidine, and roxatidine acetate (CAS No. 78628-28-1), H3 receptor antagonists (e.g., thioperamide and thioperamide maleate salt), and anti-histamines (e.g., tricyclic dibenozoxepins, ethanolamines, ethylenediamines, piperizines, piperidines, and pthalazinones), 131) an alpha adrenergic receptor antagonist, 132) an anti-psychotic compound, 133) a CaM kinase II inhibitor, 134) a G protein agonist, 135) an antibiotic selected from the group consisting of apigenin (Cas No. 520-36-5), ampicillin sodium salt (CAS No. 69-52-3), puromycin, and an analogue or derivative thereof, 136) an anti-microbial agent, 137) a DNA topoisomerase inhibitor selected from the group consisting of β-lapachone (CAS No. 4707-32-8), (−)-arctigenin (CAS No. 7770-78-7), aurintricarboxylic acid, and an analogue or derivative thereof, 138) a thromboxane A2 receptor inhibitor selected from the group consisting of BM-531 (CAS No. 284464-46-6), ozagrel hydrochloride (CAS No. 78712-43-3), and an analogue or derivative thereof, 139) a D2 dopamine receptor antagonist, 140) a Peptidyl-Prolyl Cis/Trans Isomerase Inhibitor, 141) a dopamine antagonist, an anesthetic compound, 142) a clofting factor, 143) a lysyl hydrolase inhibitor, 144) a muscarinic receptor inhibitor, 145) a superoxide anion generator, 146) a steroid, 147) an anti-proliferative agent selected from the group consisting of silibinin (CAS No. 22888-70-6), silymarin (CAS No. 65666-07-1), 1,2-hexanediol, dioctyl phthalate (CAS No. 117-81-7), zirconium (IV) oxide, glycyrrhizic acid, spermidine trihydrochloride, tetrahydrochloride, CGP 74514, spermine tetrahydrochloride, NG-methyl-L-arginine acetate salt, galardin, and an analogue or derivative thereof, 148) a diuretic, 149) an anti-coagulant, 150) a cyclic GMP agonist, 151) an adenylate cyclase agonist, 152) an antioxidant, 153) a nitric oxide synthase inhibitor, 154) an anti-neoplastic agent selected from tirapazamine (CAS No. 27314-97-2), fludarabine (CAS No. 21679-14-1), cladribine, imatinib mesilate, and an analogue or derivative thereof, 155) a DNA synthesis inhibitor, 156) a DNA alkylating agent selected from dacarbazine (CAS No. 4342-03-4), temozolomide, procarbazine HCl, and an analogue or derivative thereof, 157) a DNA methylation inhibitor, 158) a NSAID agent, 159) a peptidylglycine alpha-hydroxylating monooxygenase inhibitor, 160) an MEK1/MEK 2 inhibitor, 161) a NO synthase inhibitor, 162) a retinoic acid receptor antagonist selected from isotretinoin (CAS No. 4759-48-2) and an analogue or derivative thereof, 163) an ACE inhibitor, 164) a glycosylation inhibitor, 165) an intracellular calcium influx inhibitor, 166) an anti-emetic agent, 167) an acetylcholinesterase inhibitor, 168) an ALK-5 receptor antagonist, 169) a RAR/RXT antagonist, 170) an eIF-2a inhibitor, 171) an S-adenosyl-L-homocysteine hydrolase inhibitor, 172) an estrogen agonist, 173) a serotonin receptor inhibitor, 174) an anti-thrombotic agent, 175) a tryptase inhibitor, 176) a pesticide, 177) a bone mineralization promoter, 178) a bisphosphonate compound selected from risedronate and an analogue or derivative thereof, 179) an anti-inflammatory compound, 180) a DNA methylation promoter, 181) an anti-spasmodic agent, 182) a protein synthesis inhibitor, 183) an α-glucosidase inhibitor, 184) a calcium channel blocker, 185) a pyruvate dehydrogenase activator, 186) a prostaglandin inhibitor, 187) a sodium channel inhibitor, 188) a serine protease inhibitor, 189) an intracellular calcium flux inhibitor, 190) a JAK2 inhibitor; 191) an androgen inhibitor, 192) an aromatase inhibitor, 193) an anti-viral agent, 194) a 5-HT inhibitor, 195) an FXR antagonist, 196) an actin polymerization and stabilization promoter, 197) an AXOR12 agonist, 198) an angiotensin II receptor agonist, 199) a platelet aggregation inhibitor, 200) a CB1/CB2 receptor agonist, 201) a norepinephrine reuptake inhibitor, 202) a selective serotonin reuptake inhibitor, 203) a reducing agent, 204) Isotretinoin, 205) radicicol, 206) clobetasol propionate, 207) homoharringtonine, 208) trichostatin A, 209) brefeldin A, 210) thapsigargin, 211) dolastatin 15, 212) cerivastatin, 213) jasplakinolide, 214) herbimycin A, 215) pirfenidone, 216) vinorelbine, 217) 17-DMAG, 218) tacrolimus, 219) loteprednol etabonate, 220) juglone, 221) prednisolone, 222) puromycin, 223) 3-BAABE, 224) cladribine, 225) mannose-6-phosphate, 226) 5-azacytidine, 227) Ly333531 (ruboxistaurin), 228) simvastatin, and 229) an immuno-modulator selected from Bay 11-7085, (−)-arctigenin, idazoxan hydrochloride, and an analogue or derivative thereof. These and other agents are described in more detail herein.

In one aspect, the present invention provides a medical device, comprising an electrical device and an anti-scarring agent or a composition comprising an anti-scarring agent, wherein the agent inhibits scarring between the medical device and the host into which the medical device is implanted.

In certain embodiments, the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

In certain embodiments, the anti-scarring agent is an antimicrobial compound.

In certain embodiments, the antimicrobial compound is brefeldin A.

In certain embodiments, the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

In certain embodiments, the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

In certain embodiments, the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

In certain embodiments, the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

In certain embodiments, the medical device further comprises a coating wherein the coating comprises (a) the anti-scarring agent or (b) the anti-scarring agent and a polymer.

In certain embodiments, the medical device further comprises a coating wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

In certain embodiments, the medical device further comprises a polymer or further comprising a polymeric carrier.

In certain embodiments, the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

In certain embodiments, the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

In certain embodiments, the medical device further comprises a second pharmaceutically active agent.

In certain embodiments, the medical device further comprises at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

In certain embodiments, the medical device is adapted for delivering the anti-scarring agent locally to tissue proximate to the device.

In certain embodiments, the anti-scarring agent is released into tissue in the vicinity of the device after deployment of the device.

In certain embodiments, the anti-scarring agent is released in effective concentrations from the device over a period ranging from the time of deployment of the device to about 1 year.

In certain embodiments, the anti-scarring agent is released in effective concentrations from the device at a constant rate, an increasing rate, or a decreasing rate.

In certain embodiments, the device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

In certain embodiments, the device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

In certain embodiments, the device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

In another aspect, the present invention provides a method for inhibiting scarring comprising placing an electrical device and an anti-scarring agent or a composition comprising an ant-scarring agent into an animal host, wherein the agent inhibits scarring.

In certain embodiments, the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

In certain embodiments, the anti-scarring agent is an antimicrobial compound.

In certain embodiments, the antimicrobial compound is brefeldin A.

In certain embodiments, the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

In certain embodiments, the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

In certain embodiments, the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

In certain embodiments, the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

In certain embodiments, the electrical device further comprises a coating, and wherein the coating comprises (a) the anti-scarring agent or (b) the anti-scarring agent and a polymer.

In certain embodiments, the electrical device further comprises a coating, and wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

In certain embodiments, the electrical device further comprises a polymer or further comprises a polymeric carrier.

In certain embodiments, the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

In certain embodiments, the electrical device further comprises a polymeric carrier, and wherein the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

In certain embodiments, the electrical device further comprises a second pharmaceutically active agent.

In certain embodiments, the electrical device further comprises at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

In certain embodiments, the electrical device is adapted for delivering the anti-scarring agent locally to tissue proximate to the device.

In certain embodiments, the anti-scarring agent is released into tissue in the vicinity of the electrical device after deployment of the device.

In certain embodiments, the anti-scarring agent is released in effective concentrations from the electrical device over a period ranging from the time of deployment of the device to about 1 year.

In certain embodiments, the anti-scarring agent is released in effective concentrations from the electrical device at a constant rate, an increasing rate, or a decreasing rate.

In certain embodiments, the electrical device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

In certain embodiments, the electrical device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

In certain embodiments, the electrical device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

In another aspect, the present invention provides a method for making a medical device comprising: combining an electrical device and an anti-scarring agent or a composition comprising an anti-scarring agent, wherein the agent inhibits scarring between the device and a host into which the device is implanted.

In certain embodiments, the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

In certain embodiments, the anti-scarring agent is an antimicrobial compound.

In certain embodiments, the antimicrobial compound is brefeldin A.

In certain embodiments, the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

In certain embodiments, the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

In certain embodiments, the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

In certain embodiments, the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

In certain embodiments, the electrical device further comprises a coating, and wherein the coating comprises (a) the anti-scarring agent or (b) the anti-scarring agent and a polymer.

In certain embodiments, the electrical device further comprises a coating, and wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

In certain embodiments, the electrical device further comprises a polymer or further comprises a polymeric carrier.

In certain embodiments, the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

In certain embodiments, the electrical device further comprises a polymeric carrier, and wherein the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

In certain embodiments, the electrical device further comprises a second pharmaceutically active agent.

In certain embodiments, the electrical device further comprises at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

In certain embodiments, the electrical device is adapted for delivering the anti-scarring agent locally to tissue proximate to the device.

In certain embodiments, the anti-scarring agent is released into tissue in the vicinity of the electrical device after deployment of the device.

In certain embodiments, the anti-scarring agent is released in effective concentrations from the electrical device over a period ranging from the time of deployment of the device to about 1 year.

In certain embodiments, the anti-scarring agent is released in effective concentrations from the electrical device at a constant rate, an increasing rate, or a decreasing rate.

In certain embodiments, the electrical device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

In certain embodiments, the electrical device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

In certain embodiments, the electrical device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

In another aspect, the present invention provides a method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host.

In certain embodiments, the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

In certain embodiments, the anti-scarring agent is an antimicrobial compound.

In certain embodiments, the antimicrobial compound is brefeldin A.

In certain embodiments, the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

In certain embodiments, the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

In certain embodiments, the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

In certain embodiments, the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

In certain embodiments, the anti-infective agent is selected from an anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, and cisplatin.

In certain embodiments, the composition comprises an anti-thrombotic agent.

In certain embodiments, the polymer is formed from reactants comprising a naturally occurring polymer; protein; carbohydrate; biodegradable polymer; nonbiodegradable polymer; collagen; methylated collagen; fibrinogen; thrombin; blood plasma; calcium salt; an antifibronolytic agent; fibrinogen analog; albumin; plasminogen; von Willebrands factor; factor VIII; hypoallergenic collagen; atelopeptidic collagen; crosslinked collagen; aprotinin; epsilon-amino-n-caproic acid; gelatin; protein conjugates; gelatin conjugates; a synthetic polymer; isocyanate-containing compound; a synthetic thiol-containing compound; a synthetic compound containing at least two thiol groups; a synthetic compound containing at least three thiol groups; a synthetic compound containing at least four thiol groups; a synthetic amino-containing compound; a synthetic compound containing at least two amino groups; a synthetic compound containing at least three amino groups; a synthetic compound containing at least four amino groups; a synthetic compound comprising a carbonyl-oxygen-succinimidyl group; a synthetic compound comprising at least two carbonyl-oxygen-succinimidyl groups; a synthetic compound comprising at least three carbonyl-oxygen-succinimidyl groups; a synthetic compound comprising at least four carbonyl-oxygen-succinimidyl groups; a synthetic polyalkylene oxide-containing compound; a synthetic compound comprising both polyalkylene oxide and biodegradable polyester blocks; a synthetic polyalkylene oxide-containing compound having reactive amino groups; a synthetic polyalkylene oxide-containing compound having reactive thiol groups; a synthetic polyalkylene oxide-containing compound having reactive carbonyl-oxygen-succinimidyl groups; a synthetic compound comprising a biodegradable polyester block; a synthetic polymer formed in whole or part from lactic acid or lactide; a synthetic polymer formed in whole or part from glycolic acid or glycolide; polylysine; (a) protein and (b) a compound comprising a polyalkylene oxide portion; (a) protein and (b) polylysine; (a) protein and (b) a compound having at least four thiol groups; (a) protein and (b) a compound having at least four amino groups; (a) protein and (b) a compound having at least four carbonyl-oxygen-succinimide groups; (a) protein and (b) a compound having a biodegradable region formed from reactants selected from lactic acid, lactide, glycolic acid, glycolide, and epison-caprolactone; (a) collagen and (b) a compound comprising a polyalkylene oxide portion; (a) collagen and (b) polylysine; (a) collagen and (b) a compound having at least four thiol groups; (a) collagen and (b) a compound having at least four thiol groups; (a) collagen and (b) a compound having at least four amino groups; (a) collagen and (b) a compound having at least four carbonyl-oxygen-succinimide groups; (a) collagen and (b) a compound having a biodegradable region formed from reactants selected from lactic acid, lactide, glycolic acid, glycolide, and epison-caprolactone; (a) methylated collagen and (b) a compound comprising a polyalkylene oxide portion; (a) methylated collagen and (b) polylysine; (a) methylated collagen and (b) a compound having at least four thiol groups; (a) methylated collagen and (b) a compound having at least four amino groups; (a) methylated collagen and (b) a compound having at least four carbonyl-oxygen-succinimide groups; (a) methylated collagen and (b) a compound having a biodegradable region formed from reactants selected from lactic acid, lactide, glycolic acid, glycolide, and epison-caprolactone; hyaluronic acid; a hyaluronic acid derivative; pentaerythritol poly(ethylene glycol)ether tetra-sulfhydryl of number average molecular weight between 3,000 and 30,000; pentaerythritol poly(ethylene glycol)ether tetra-amino of number average molecular weight between 3,000 and 30,000; or (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

These and other aspects of the present invention will become evident upon reference to the following detailed description and attached drawings. In addition, various references are set forth herein which describe in more detail certain procedures and/or compositions (e.g., polymers), and are therefore incorporated by reference in their entirety.

### BRIEF DESCRIPTION OF THE DRAWINGS

FIG. 1 is a picture that shows an uninjured carotid artery from a rat balloon injury model.

FIG. 2 is a picture that shows an injured carotid artery from a rat balloon injury model.

FIG. 3 is a picture that shows a paclitaxel/mesh treated carotid artery in a rat balloon injury model.

FIG. 4A schematically depicts the transcriptional regulation of matrix metalloproteinases.

FIG. 4B is a blot which demonstrates that IL-1 stimulates AP-1 transcriptional activity.

FIG. 4C is a graph which shows that IL-1 induced binding activity decreased in lysates from chondrocytes which were pretreated with paclitaxel.

FIG. 4D is a blot which shows that IL-1 induction increases collagenase and stromelysin in RNA levels in chondrocytes, and that this induction can be inhibited by pretreatment with paclitaxel.

FIGS. 5A-H are blots that show the effect of various anti-microtubule agents in inhibiting collagenase expression.

FIG. 6 is a graph showing the results of a screening assay for assessing the effect of paclitaxel on smooth muscle cell migration.

FIG. 7 is a bar graph showing the area of granulation tissue in carotid arteries exposed to silk coated perivascular polyurethane (PU) films relative to arteries exposed to uncoated PU films.

FIG. 8 is a bar graph showing the area of granulation tissue in carotid arteries exposed to silk suture coated perivascular PU films relative to arteries exposed to uncoated PU films.

FIG. 9 is a bar graph showing the area of granulation tissue in carotid arteries exposed to natural and purified silk powder and wrapped with perivascular PU film relative to a control group in which arteries are wrapped with perivascular PU film only.

FIG. 10 is a bar graph showing the area of granulation tissue (at 1 month and 3 months) in carotid arteries sprinkled with talcum powder and wrapped with perivascular PU film relative to a control group in which arteries are wrapped with perivascular PU film only.

### DETAILED DESCRIPTION OF THE INVENTION

### Definitions

Prior to setting forth the invention, it may be helpful to an understanding thereof to first set forth definitions of certain terms that is used hereinafter.

“Medical device”, “implant”, “medical device or implant”, “implant/device”, “the device”, and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing or replacing or augmenting etc. damaged or diseased organs and tissues. While medical devices are normally composed of biologically compatible synthetic materials (e.g., medical-grade stainless steel, titanium and other metals; exogenous polymers, such as polyurethane, silicon, PLA, PLGA), other materials may also be used in the construction of the medical device or implant. Specific medical devices and implants that are particularly useful for the practice of this invention include devices and implants that are used to provide electrical stimulation to the central and peripheral nervous system (including the autonomic system), cardiac muscle tissue (including myocardial conduction pathways), smooth muscle tissue and skeletal muscle tissue.

“Electrical device” refers to a medical device having electrical components that can be placed in contact with tissue in an animal host and can provide electrical excitation to nervous or muscular tissue. Electrical devices can generate electrical impulses and may be used to treat many bodily dysfunctions and disorders by blocking, masking, or stimulating electrical signals within the body. Electrical medical devices of particular utility in the present invention include, but are not restricted to, devices used in the treatment of cardiac rhythm abnormalities, pain relief, epilepsy, Parkinson's Disease, movement disorders, obesity, depression, anxiety and hearing loss.

“Neurostimulator” or “Neurostimulation Device” refers to an electrical device for electrical excitation of the central, autonomic, or peripheral nervous system. The neurostimulator sends electrical impulses to an organ or tissue. The neurostimulator may include electrical leads as part of the electrical stimulation system. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics.

“Cardiac Stimulation Device” or “Cardiac Rhythm Management Device” or “Cardiac Pacemaker” or “Implantable Cardiac Defibrillator (ICD)” all refer to an electrical device for electrical excitation of cardiac muscle tissue (including the specialized cardiac muscle cells that make up the conductive pathways of the heart). The cardiac pacemaker sends electrical impulses to the muscle (myocardium) or conduction tissue of the heart. The pacemaker may include electrical leads as part of the electrical stimulation system. Cardiac pacemakers may be used to block, mask, or stimulate electrical signals in the heart to treat dysfunctions, including, without limitation, atrial rhythm abnormalities, conduction abnormalities and ventricular rhythm abnormalities.

“Electrical lead” refers to an electrical device that is used as a conductor to carry electrical signals from the generator to the tissues. Typically, electrical leads are composed of a connector assembly, a lead body (i.e., conductor) and an electrode. The electrical lead may be a wire or other material that transmits electrical impulses from a generator (e.g., pacemaker, defibrillator, or other neurostimulator). Electrical leads may be unipolar, in which they are adapted to provide effective therapy with only one electrode. Multi-polar leads are also available, including bipolar, tripolar and quadripolar leads.

“Fibrosis” or “Scarring” refers to the formation of fibrous (scar) tissue (or in the case of injury in the CNS—the formation of glial tissue, or “gliosis”, by astrocytes) in response to injury or medical intervention. Therapeutic agents which inhibit fibrosis or scarring (referred to as “anti-fibrotic agents,” “anti-fibrosis agents,” “anti-scarring agents,” “fibrosis-inhibiting agents,” or the like) can do so through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling. Therapeutic agents which inhibit gliosis (referred to as “anti-gliosis agents,” “anti-gliotic agents,” “gliosis-inhibiting agents,” or the like) can do so through one or more mechanisms including: inhibiting migration of glial cells, inhibition of hypertrophy of glial cells, and/or inhibiting proliferation of glial cells. In addition, numerous therapeutic agents described in this invention may have the additional benefit of also reducing tissue regeneration (the replacement of injured cells by cells of the same type) when appropriate.

“Inhibit fibrosis”, “reduce fibrosis”, “inhibit gliosis”, “reduce gliosis” and the like are used synonymously to refer to the action of agents or compositions which result in a statistically significant decrease in the formation of fibrous or glial tissue that may be expected to occur in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett. 79(2):213-219, 1994) and Mooberry et al., (Cancer Lett. 96(2):261-266, 1995).

“Host”, “Person”, “Subject”, “Patient” and the like are used synonymously to refer to the living being (human or animal) into which a device of the present invention is implanted.

“Implanted” refers to having completely or partially placed a device within a host. A device is partially implanted when some of the device reaches, or extends to the outside of, a host.

“Release of an agent” refers to a statistically significant presence of the agent, or a subcomponent thereof, which has disassociated from the implant/device and/or remains active on the surface of (or within) the device/implant.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

Any concentration ranges, percentage ranges, or ratio ranges recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers. As used herein, the term “about” means±15%.

As discussed above, the present invention provides compositions, methods and devices relating to medical devices and implants, which greatly increase their ability to inhibit the formation of reactive scar (or glial) tissue on, or around, the surface of the device or implant. Described in more detail below are methods for constructing medical devices or implants, compositions and methods for generating medical devices and implants which inhibit fibrosis, and methods for utilizing such medical devices and implants.

A. Clinical Applications of Electrical Medical Devices and Implants which Contain a Fibrosis-Inhibiting Agent

Medical devices having electrical components, such as electrical pacing or stimulating devices, can be implanted in the body to provide electrical conduction to the central and peripheral nervous system (including the autonomic system), cardiac muscle tissue (including myocardial conduction pathways), smooth muscle tissue and skeletal muscle tissue. These electrical impulses are used to treat many bodily dysfunctions and disorders by blocking, masking, stimulating, or replacing electrical signals within the body. Examples include pacemaker leads used to maintain the normal rhythmic beating of the heart; defibrillator leads used to “re-start” the heart when it stops beating; peripheral nerve stimulating devices to treat chronic pain; deep brain electrical stimulation to treat conditions such as tremor, Parkinson's disease, movement disorders, epilepsy, depression and psychiatric disorders; and vagal nerve stimulation to treat epilepsy, depression, anxiety, obesity, migraine and Alzheimer's Disease.

The clinical function of an electrical device such as a cardiac pacemaker lead, neurostimulation lead, or other electrical lead depends upon the device being able to effectively maintain intimate anatomical contact with the target tissue (typically electrically excitable cells such as muscle or nerve) such that electrical conduction from the device to the tissue can occur. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue (or glial tissue—called “gliosis”—when it occurs within the central nervous system). Scarring (i.e., fibrosis or gliosis) can also result from trauma to the anatomical structures and tissue surrounding the implant during the implantation of the device. Lastly, fibrous encapsulation of the device can occur even after a successful implantation if the device is manipulated (some patients continuously “fiddle” with a subcutaneous implant) or irritated by the daily activities of the patient. When scarring occurs around the implanted device, the electrical characteristics of the electrode-tissue interface degrade, and the device may fail to function properly. For example, it may require additional electrical current from the lead to overcome the extra resistance imposed by the intervening scar (or glial) tissue. This can shorten the battery life of an implant (making more frequent removal and re-implantation necessary), prevent electrical conduction altogether (rendering the implant clinically ineffective) and/or cause damage to the target tissue. Additionally, the surrounding tissue may be inadvertently damaged from the inflammatory foreign body response, which can result in loss of function or tissue necrosis.

The present invention addresses these problems. Exemplary electrical devices are described next.

1) Neurostimulation Devices

In one aspect, the electrical device may be a neurostimulation device where a pulse generator delivers an electrical impulse to a nervous tissue (e.g., CNS, peripheral nerves, autonomic nerves) in order to regulate its activity. There are numerous neurostimulator devices where the occurrence of a fibrotic reaction may adversely affect the functioning of the device or the biological problem for which the device was implanted or used. Typically, fibrotic encapsulation of the electrical lead (or the growth of fibrous tissue between the lead and the target nerve tissue) slows, impairs, or interrupts electrical transmission of the impulse from the device to the tissue. This can cause the device to function suboptimally or not at all, or can cause excessive drain on battery life because increased energy is required to overcome the electrical resistance imposed by the intervening scar (or glial) tissue.

Neurostimulation devices are used as alternative or adjunctive therapy for chronic, neurodegenerative diseases, which are typically treated with drug therapy, invasive therapy, or behavioral/lifestyle changes. Neurostimulation may be used to block, mask, or stimulate electrical signals in the body to treat dysfunctions, including, without limitation, pain, seizures, anxiety disorders, depression, ulcers, deep vein thrombosis, muscular atrophy, obesity, joint stiffness, muscle spasms, osteoporosis, scoliosis, spinal disc degeneration, spinal cord injury, deafness, urinary dysfunction and gastroparesis. Neurostimulation may be delivered to many different parts of the nervous system, including, spinal cord, brain, vagus nerve, sacral nerve, gastric nerve, auditory nerves, as well as organs, bone, muscles and tissues. As such, neurostimulators are developed to conform to the different anatomical structures and nervous system characteristics. Representative examples of neurologic and neurosurgical implants and devices that can be coated with, or otherwise constructed to contain and/or release the therapeutic agents provided herein, include, e.g., nerve stimulator devices to provide pain relief, devices for continuous subarachnoid infusions, implantable electrodes, stimulation electrodes, implantable pulse generators, electrical leads, stimulation catheter leads, neurostimulation systems, electrical stimulators, cochlear implants, auditory stimulators and microstimulators.

Neurostimulation devices may also be classified based on their source of power, which includes: battery powered, radio-frequency (RF) powered, or a combination of both types. For battery powered neurostimulators, an implanted, non-rechargeable battery is used for power. The battery and leads are all surgically implanted and thus the neurostimulation device is completely internal. The settings of the totally implanted neurostimulator are controlled by the patient through an external magnet. The lifetime of the implant is generally limited by the duration of battery life and ranges from two to four years depending upon usage and power requirements. For RF-powered neurostimulation devices, the radio-frequency is transmitted from an externally worn source to an implanted passive receiver. Since the power source is readily rechargeable or replaceable, the radio-frequency system enables greater power resources and thus, multiple leads may be used in these systems. Specific examples include a neurostimulator that has a battery power source contained within to supply power over an eight hour period in which power may be replenished by an external radio frequency coupled device (See e.g., U.S. Pat. No. 5,807,397) or a microstimulator which is controlled by an external transmitter using data signals and powered by radio frequency (See e.g., U.S. Pat. No. 6,061,596).

Examples of commercially available neurostimulation products include a radio-frequency powered neurostimulator comprised of the 3272 MATTRIX Receiver, 3210 MATTRIX Transmitter and 3487A PISCES-QUAD Quadripolar Leads made by Medtronic, Inc. (Minneapolis, Minn.). Medtronic also sells a battery-powered ITREL 3 Neurostimulator and SYNERGY Neurostimulator, the INTERSIM Therapy for sacral nerve stimulation for urinary control, and leads such as the 3998 SPECIFY Lead and 3587A RESUME II Lead.

Another example of a neurostimulation device is a gastric pacemaker, in which multiple electrodes are positioned along the GI tract to deliver a phased electrical stimulation to pace peristaltic movement of the material through the GI tract. See, e.g., U.S. Pat. No. 5,690,691. A representative example of a gastric stimulation device is the ENTERRA Gastric Electrical Stimulation (GES) from Medtronic, Inc. (Minneapolis, Minn.).

The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices (as described previously). Neurostimulator devices that release a therapeutic agent for reducing scarring (or gliosis) at the electrode-tissue interface can be used to increase the efficacy and/or the duration of activity (particularly for fully-implanted, battery-powered devices) of the implant. Accordingly, the present invention provides neurostimulator leads that are coated with an anti-scarring agent or a composition that includes an anti-scarring (or anti-gliosis) agent.

For greater clarity, several specific neurostimulation devices and treatments will be described in greater detail including:

a) Neurostimulation for the Treatment of Chronic Pain

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 40 million Americans are afflicted with recurrent headaches and that the cost of medications for this condition exceeds $4 billion a year. A further 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, neurostimulation devices have been developed to treat severe intractable pain that is resistant to other traditional treatment modalities such as drug therapy, invasive therapy (surgery), or behavioral/lifestyle changes.

In principle, neurostimulation works by delivering low voltage electrical stimulation to the spinal cord or a particular peripheral nerve in order to block the sensation of pain. The Gate Control Theory of Pain (Ronald Melzack and Patrick Wall) hypothesizes that there is a “gate” in the dorsal horn of the spinal cord that controls the flow of pain signals from the peripheral receptors to the brain. It is speculated that the body can inhibit the pain signals (“close the gate”) by activating other (non-pain) fibers in the region of the dorsal horn. Neurostimulation devices are implanted in the epidural space of the spinal cord to stimulate non-noxious nerve fibers in the dorsal horn and mask the sensation of pain. As a result the patient typically experiences a tingling sensation (known as paresthesia) instead of pain. With neurostimulation, the majority of patients will report improved pain relief (50% reduction), increased activity levels and a reduction in the use of narcotics.

Pain management neurostimulation systems consist of a power source that generates the electrical stimulation, leads (typically 1 or 2) that deliver electrical stimulation to the spinal cord or targeted peripheral nerve, and an electrical connection that connects the power source to the leads. Neurostimulation systems can be battery powered, radio-frequency powered, or a combination of both. In general, there are two types of neurostimulation devices: those that are surgically implanted and are completely internal (i.e., the battery and leads are implanted), and those with internal (leads and radio-frequency receiver) and external (power source and antenna) components. For internal, battery-powered neurostimulators, an implanted, non-rechargeable battery and the leads are all surgically implanted. The settings of the totally implanted neurostimulator may be controlled by the host by using an external magnet and the implant has a lifespan of two to four years. For radio-frequency powered neurostimulators, the radio-frequency is transmitted from an externally worn source to an implanted passive receiver. The radio-frequency system enables greater power resources and thus, multiple leads may be used.

There are numerous neurostimulation devices that can be used for spinal cord stimulation in the management of pain control, postural positioning and other disorders. Examples of specific neurostimulation devices include those composed of a sensor that detects the position of the spine and a stimulator that automatically emits a series of pulses which decrease in amplitude when back is in a supine position. See e.g., U.S. Pat. Nos. 5,031,618 and 5,342,409. The neurostimulator may be composed of electrodes and a control circuit which generates pulses and rest periods based on intervals corresponding to the body's activity and regeneration period as a treatment for pain. See e.g., U.S. Pat. No. 5,354,320. The neurostimulator, which may be implanted within the epidural space parallel to the axis of the spinal cord, may transmit data to a receiver which generates a spinal cord stimulation pulse that may be delivered via a coupled, multi-electrode. See e.g., U.S. Pat. No. 6,609,031. The neurostimulator may be a stimulation catheter lead with a sheath and at least three electrodes that provide stimulation to neural tissue. See e.g., U.S. Pat. No. 6,510,347. The neurostimulator may be a self-centering epidual spinal cord lead with a pivoting region to stabilize the lead which inflates when injected with a hardening agent. See e.g., U.S. Pat. No. 6,308,103. Other neurostimulators used to induce electrical activity in the spinal cord are described in, e.g., U.S. Pat. Nos. 6,546,293; 6,236,892; 4,044,774 and 3,724,467.

Commercially available neurostimulation devices for the management of chronic pain include the SYNERGY, INTREL, X-TREL and MATTRIX neurostimulation systems from Medtronic, Inc. The percutaneous leads in this system can be quadripolar (4 electrodes), such as the PISCES-QUAD, PISCES-QUAD PLUS and the PISCES-QUAD Compact, or octapolar (8 electrodes) such as the OCTAD lead. The surgical leads themselves are quadripolar, such as the SPECIFY Lead, the RESUME II Lead, the RESUME TL Lead and the ON-POINT PNS Lead, to create multiple stimulation combinations and a broad area of paresthesia. These neurostimulation systems and associated leads may be described, for example, in U.S. Pat. Nos. 6,671,544; 6,654,642; 6,360,750; 6,353,762; 6,058,331; 5,342,409; 5,031,618 and 4,044,774. Neurostimulating leads such as these may benefit from release of a therapeutic agent able to reducing scarring at the electrode-tissue interface to increase the efficiency of impulse transmission and increase the duration that the leads function clinically. In one aspect, the device includes spinal cord stimulating devices and/or leads that are coated with an anti-scarring (or anti-gliosis) agent or a composition that includes an anti-scarring (or anti-gliosis) agent. As an alternative to this, or in addition to this, a composition that includes an anti-scarring agent can be infiltrated into the epidural space where the lead will be implanted. Other commercially available systems that may useful for the practice of this invention as described above include the rechargeable PRECISION Spinal Cord Stimulation System (Advanced Bionics Corporation, Sylmar, Calif.; which is a Boston Scientific Company) which can drive up to 16 electrodes (see e.g., U.S. Pat. Nos. 6,735,474; 6,735,475; 6,659,968; 6,622,048; 6,516,227 and 6,052,624). The GENESIS XP Spinal Cord Stimulator available from Advanced Neuromodulation Systems, Inc. (Piano, Tex.; see e.g., U.S. Pat. Nos. 6,748,276; 6,609,031 and 5,938,690) as well as the Vagus Nerve Stimulation (VNS) Therapy System available from Cyberonics, Inc. (Houston, Tex.; see e.g., U.S. Pat. Nos. 6,721,603 and 5,330,515) may also benefit from the application of anti-fibrosis (or anti-gliosis) agents as described in this invention.

Regardless of the specific design features, for neurostimulation to be effective in pain relief, the leads must be accurately positioned adjacent to the portion of the spinal cord or the targeted peripheral nerve that is to be electrically stimulated. Neurostimulators can migrate following surgery or excessive tissue growth or extracellular matrix deposition can occur around neurostimulators, which can lead to a reduction in the functioning of these devices. Neurostimulator devices that release therapeutic agent for reducing scarring at the electrode-tissue interface can be used to increase the duration that these devices clinically function. In one aspect, the device includes neurostimulator devices and/or leads that are coated with an anti-scarring (or anti-gliosis) agent or a composition that includes an anti-scarring (or anti-gliosis) agent. As an alternative to this, or in addition to this, a composition that includes an anti-scarring (anti-gliosis) agent can be infiltrated into the tissue surrounding the implanted portion (particularly the leads) of the pain management neurostimulation device.

b) Neurostimulation for the Treatment of Parkinson's Disease

Neurostimulation devices implanted into the brain are used to control the symptoms associated with Parkinson's disease or essential tremor. Typically, these are dual chambered stimulator devices (similar to cardiac pacemakers) that deliver bilateral stimulation to parts of the brain that control motor function. Electrical stimulation is used to relieve muscular symptoms due to Parkinson's disease itself (tremor, rigidity, bradykinesia, akinesia) or symptoms that arise as a result of side effects of the medications used to treat the disease (dyskinesias). Two stimulating electrodes are implanted in the brain (usually bilaterally in the subthalamic nucleus or the globus pallidus interna) for the treatment of levodopa-responsive Parkinson's and one is implanted (in the ventral intermediate nucleus of the thalamus) for the treatment of tremor. The electrodes are implanted in the brain by a functional stereotactic neurosurgeon using a stereotactic head frame and MRI or CT guidance. The electrodes are connected via extensions (which run under the skin of the scalp and neck) to a neurostimulatory (pulse generating) device implanted under the skin near the clavicle. A neurologist can then optimize symptom control by adjusting stimulation parameters using a noninvasive control device that communicates with the neurostimulator via telemetry. The patient is also able to turn the system on and off using a magnet and control the device (within limits set by the neurologist) settings using a controller device. This form of deep brain stimulation has also been investigated for the treatment pain, epilepsy, psychiatric conditions (obsessive-compulsive disorder) and dystonia.

Several devices have been described for such applications including, for example, a neurostimulator and an implantable electrode that has a flexible, non-conducting covering material, which is used for tissue monitoring and stimulation of the cortical tissue of the brain as well as other tissue. See e.g., U.S. Pat. No. 6,024,702. The neurostimulator (pulse generator) may be an intracranially implanted electrical control module and a plurality of electrodes which stimulate the brain tissue with an electrical signal at a defined frequency. See e.g., U.S. Pat. No. 6,591,138. The neurostimulator may be a system composed of at least two electrodes adapted to the cranium and a control module adapted to be implanted beneath the scalp for transmitting output electrical signals and also external equipment for providing two-way communication. See e.g., U.S. Pat. No. 6,016,449. The neurostimulator may be an implantable assembly composed of a sensor and two electrodes, which are used to modify the electrical activity in the brain. See e.g., U.S. Pat. No. 6,466,822.

A commercial example of a device used to treat Parkinson's disease and essential tremor includes the ACTIVA System by Medtronic, Inc. (see, for example, U.S. Pat. Nos. 6,671,544 and 6,654,642). This system consists of the KINETRA Dual Chamber neurostimulator, the SOLETRA neurostimulator or the INTREL neurostimulator, connected to an extension (an insulated wire), that is further connected to a DBS lead. The DBS lead consists of four thin, insulated, coiled wires bundled with polyurethane. Each of the four wires ends in a 1.5 mm long electrode. Although all or parts of the DBS lead may be suitable for coating with a fibrosis/gliosis-inhibiting composition, a preferred embodiment involves delivering the therapeutic agent from the surface of the four electrodes. As an alternative to this, or in addition to this, a composition that includes an anti-gliosis agent can be infiltrated into the brain tissue surrounding the leads.

c) Vagal Nerve Stimulation for the Treatment of Epilepsy

Neurostimulation devices are also used for vagal nerve stimulation in the management of pharmacoresistant epilepsy (i.e., epilepsy that is uncontrolled despite appropriate medical treatment with ant-epileptic drugs). Approximately 30% of epileptic patients continue to have seizures despite of multiple attempts at controlling the disease with drug therapy or are unable to tolerate the side effects of their medications. It is estimated that approximately 2.5 million patients in the United States suffer from treatment-resistant epilepsy and may benefit from vagal nerve stimulation therapy. As such, inadequate seizure control remains a significant medical problem with many patients suffering from diminished self esteem, poor academic achievement and a restricted lifestyle as a result of their illness.

The vagus nerve (also called the 10^{th} cranial nerve) contains primarily afferent sensory fibres that carry information from the neck, thorax and abdomen to the nucleus tractus soltarius of the brainstem and on to multiple noradrenergic and serotonergic neuromodulatory systems in the brain and spinal cord. Vagal nerve stimulation (VNS) has been shown to induce progressive EEG changes, alter bilateral cerebral blood flow, and change blood flow to the thalamus. Although the exact mechanism of seizure control is not known, VNS has been demonstrated clinically to terminate seizures after seizure onset, reduce the severity and frequency of seizures, prevent seizures when used prophylactically over time, improve quality of life, and reduce the dosage, number and side effects of anti-epileptic medications (resulting in improved alertness, mood, memory).

In VNS, a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the neurologist to suit an individual patient's symptoms, while the patient can turn the device on and off through the use of an external magnet. Chronic electrical stimulation which can be used as a direct treatment for epilepsy is described in, for example, U.S. Pat. No. 6,016,449, whereby, an implantable neurostimulator is coupled to relatively permanent deep brain electrodes. The implantable neurostimulator may be composed of an implantable electrical lead having a furcated, or split, distal portion with two or more separate end segments, each of which bears at least one sensing or stimulation electrode, which may be used to treat epilepsy and other neurological disorders. See e.g., U.S. Pat. No. 6,597,953.

A commercial example of a VNS system is the product produced by Cyberonics, Inc. that includes the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,540,730 and 5,299,569.

Regardless of the specific design features, for vagal nerve stimulation to be effective in epilepsy, the leads must be accurately positioned adjacent to the left vagus nerve. If excessive scar tissue growth or extracellular matrix deposition occurs around the VNS leads, this can reduce the efficacy of the device. VNS devices that release a therapeutic agent able to reducing scarring at the electrode-tissue interface can increase the efficiency of impulse transmission and increase the duration that these devices function clinically. In one aspect, the device includes VNS devices and/or leads that are coated with an anti-scarring agent or a composition that includes an anti-scarring agent. As an alternative to this, or in addition to this, a composition that includes an anti-scarring agent can be infiltrated into the tissue surrounding the vagus nerve where the lead will be implanted.

d) Vagal Nerve Stimulation for the Treatment of Other Disorders

It was discovered during the use of VNS for the treatment of epilepsy that some patients experienced an improvement in their mood during therapy. As such, VNS is currently being examined for use in the management of treatment-resistant mood disorders such as depression and anxiety. Depression remains an enormous clinical problem in the Western World with over 1% (25 million people in the United States) suffering from depression that is inadequately treated by pharmacotherapy. Vagal nerve stimulation has been examined in the management of conditions such as anxiety (panic disorder, obsessive-compulsive disorder, post-traumatic stress disorder), obesity, migraine, sleep disorders, dementia, Alzheimer's disease and other chronic or degenerative neurological disorders. VNS has also been examined for use in the treatment of medically significant obesity.

The implantable neurostimulator for the treatment of neurological disorders may be composed of an implantable electrical lead having a furcated, or split, distal portion with two or more separate end segments, each of which bears at least one sensing or stimulation electrode. See e.g., U.S. Pat. No. 6,597,953. The implantable neurostimulator may be an apparatus for treating Alzheimer's disease and dementia, particularly for neuro modulating or stimulating left vagus nerve, composed of an implantable lead-receiver, external stimulator, and primary coil. See e.g., U.S. Pat. No. 6,615,085.

Cyberonics, Inc. manufactures the commercially available VNS system, including the Model 300 and Model 302 leads, the Model 101 and Model 102R pulse generators, the Model 201 programming wand and Model 250 programming software, and the Model 220 magnets. These products as well as others that are being developed by Cyberonics, Inc. may be used to treat neurological disorders, including depression (see e.g., U.S. Pat. No. 5,299,569), dementia (see e.g., U.S. Pat. No. 5,269,303), migraines (see e.g., U.S. Pat. No. 5,215,086), sleep disorders (see e.g., U.S. Pat. No. 5,335,657) and obesity (see e.g., U.S. Pat. Nos. 6,587,719; 6,609,025; 5,263,480 and 5,188,104).

It is important to note that the fundamentals of treatment are identical to those described above for epilepsy. The devices employed and the principles of therapy are also similar. As was described above for the treatment of epilepsy, if excessive scar tissue growth or extracellular matrix deposition occurs around the VNS leads, this can reduce the efficacy of the device. VNS devices that release a therapeutic agent able to reducing scarring at the electrode-tissue interface can increase the efficiency of impulse transmission and increase the duration that these devices function clinically for the treatment of depression, anxiety, obesity, sleep disorders and dementia. In one aspect, the device includes VNS devices and/or leads that are coated with an anti-scarring agent or a composition that includes an anti-scarring agent. As an alternative to this, or in addition to this, a composition that includes an anti-scarring agent can be infiltrated into the tissue surrounding the vagus nerve where the lead will be implanted.

e) Sacral Nerve Stimulation for Bladder Control Problems

Sacral nerve stimulation is used in the management of patients with urinary control problems such as urge incontinence, nonobstructive urinary retention, or urgency-frequency. Millions of people suffer from bladder control problems and a significant percentage (estimated to be in excess of 60%) is not adequately treated by other available therapies such as medications, absorbent pads, external collection devices, bladder augmentation or surgical correction. This can be a debilitating medical problem that can cause severe social anxiety and cause people to become isolated and depressed.

Mild electrical stimulation of the sacral nerve is used to influence the functioning of the bladder, urinary sphincter, and the pelvic floor muscles (all structures which receive nerve supply from the sacral nerve). An electrical lead is surgically implanted adjacent to the sacral nerve and a neurostimulator is implanted subcutaneously in the upper buttock or abdomen; the two are connected by an extension. The use of tined leads allows sutureless anchoring of the leads and minimally-invasive placement of the leads under local anesthesia. A handheld programmer is available for adjustment of the device by the attending physician and a patient-controlled programmer is available to adjust the settings and to turn the device on and off. The pulses are adjusted to provide bladder control and relieve the patient's symptoms.

Several neurostimulation systems have been described for sacral nerve stimulation in which electrical stimulation is targeted towards the bladder, pelvic floor muscles, bowel and/or sexual organs. For example, the neurostimulator may be an electrical stimulation system composed of an electrical stimulator and leads having insulator sheaths, which may be anchored in the sacrum using minimally-invasive surgery. See e.g., U.S. Pat. No. 5,957,965. In another aspect, the neurostimulator may be used to condition pelvic, sphincter or bladder muscle tissue. For example, the neurostimulator may be intramuscular electrical stimulator composed of a pulse generator and an elongated medical lead that is used for electrically stimulating or sensing electrical signals originating from muscle tissue. See e.g., U.S. Pat. No. 6,434,431. Another neurostimulation system consists of a leadless, tubular-shaped microstimulator that is implanted at pelvic floor muscles or associated nerve tissue that need to be stimulated to treat urinary incontinence. See e.g., U.S. Pat. No. 6,061,596.

A commercially available example of a neurostimulation system to treat bladder conditions is the INTERSTIM Sacral Nerve Stimulation System made by Medtronic, Inc. See e.g., U.S. Pat. Nos. 6,104,960; 6,055,456 and 5,957,965.

Regardless of the specific design features, for bladder control therapy to be effective, the leads must be accurately positioned adjacent to the sacral nerve, bladder, sphincter or pelvic muscle (depending upon the particular system employed). If excessive scar tissue growth or extracellular matrix deposition occurs around the leads, efficacy can be compromised. Sacral nerve stimulating devices (such as INTERSTIM) that release a therapeutic agent able to reducing scarring at the electrode-tissue interface can increase the efficiency of impulse transmission and increase the duration that these devices function clinically. In one aspect, the device includes sacral nerve stimulating devices and/or leads that are coated with an anti-scarring agent or a composition that includes an anti-scarring agent. As an alternative to this, or in addition to this, a composition that includes an anti-scarring agent can be infiltrated into the tissue surrounding the sacral nerve where the lead will be implanted.

For devices designed to stimulate the bladder or pelvic muscle tissue directly, slightly different embodiments may be required. In this aspect, the device includes bladder or pelvic muscle stimulating devices, leads, and/or sensors that are coated with an anti-scarring agent or a composition that includes an anti-scarring agent. As an alternative to this, or in addition to this, a composition that includes an anti-scarring agent can be directly infiltrated into the muscle tissue itself (preferably adjacent to the lead and/or sensor that is delivering an impulse or monitoring the activity of the muscle).

f) Gastric Nerve Stimulation for the Treatment of GI Disorders

Neurostimulator of the gastric nerve (which supplies the stomach and other portions of the upper GI tract) is used to influence gastric emptying and satiety sensation in the management of clinically significant obesity or problems associated with impaired GI motility. Morbid obesity has reached epidemic proportions and is thought to affect over 25 million Americans and lead to significant health problems such as diabetes, heart attack, stroke and death. Mild electrical stimulation of the gastric nerve is used to influence the functioning of the upper GI tract and stomach (all structures which receive nerve supply from the gastric nerve). An electrical lead is surgically implanted adjacent to the gastric nerve and a neurostimulator is implanted subcutaneously; the two are connected by an extension. A handheld programmer is available for adjustment of the device by the attending physician and a patient-controlled programmer is available to adjust the settings and to turn the device on and off. The pulses are adjusted to provide a sensation of satiety and relieve the sensation of hunger experienced by the patient. This can reduce the amount of food (and hence caloric) intake and allow the patient to lose weight successfully. Related devices include neurostimulation devices used to stimulate gastric emptying in patients with impaired gastric motility, a neurostimulator to promote bowel evacuation in patients with constipation (stimulation is delivered to the colon), and devices targeted at the bowel for patients with other GI motility disorders.

Several such devices have been described including, for example, a sensor that senses electrical activity in the gastrointestinal tract which is coupled to a pulse generator that emits and inhibits asynchronous stimulation pulse trains based on the natural gastrointestinal electrical activity. See e.g., U.S. Pat. No. 5,995,872. Other neurostimulation devices deliver impulses to the colon and rectum to manage constipation and are composed of electrical leads, electrodes and an implanted stimulation generator. See e.g., U.S. Pat. No. 6,026,326. The neurostimulator may be a pulse generator and electrodes that electrically stimulate the neuromuscular tissue of the viscera to treat obesity. See e.g., U.S. Pat. No. 6,606,523. The neurostimulator may be a hermetically sealed implantable pulse generator that is electrically coupled to the gastrointestinal tract and emits two rates of electrical stimulation to treat gastroparesis for patients with impaired gastric emptying. See e.g., U.S. Pat. No. 6,091,992. The neurostimulator may be composed of an electrical signal controller, connector wire and attachment lead which generates continuous low voltage electrical stimulation to the fundus of the stomach to control appetite. See e.g., U.S. Pat. No. 6,564,101. Other neurostimulators that are used to electrically stimulate the gastrointestinal tract are described in, e.g., U.S. Pat. Nos. 6,453,199; 6,449,511 and 6,243,607.

Another example of a gastric nerve stimulation device for use with the present invention is the TRANSCEND Implantable Gastric Stimulator (IGS), which is currently being developed by Transneuronix, Inc. (Mt. Arlington, N.J.). The IGS is a programmable, bipolar pulse generator that delivers small bursts of electrical pulses through the lead to the stomach wall to treat obesity. See, e.g., U.S. Pat. Nos. 6,684,104 and 6,165,084.

Regardless of the specific design features, for gastric nerve stimulation to be effective in satiety control (or gastroparesis), the leads must be accurately positioned adjacent to the gastric nerve. If excessive scar tissue growth or extracellular matrix deposition occurs around the leads, efficacy can be compromised. Gastric nerve stimulating devices (and other implanted devices designed to influence GI motility) that release a therapeutic agent able to reduce scarring at the electrode-tissue interface can increase the efficiency of impulse transmission and increase the duration that these devices function clinically. In one aspect, the device includes gastric nerve stimulating devices and/or leads that are coated with an anti-scarring agent or a composition that includes an anti-scarring agent. As an alternative to this, or in addition to this, a composition that includes an anti-scarring agent can be infiltrated into the tissue surrounding the gastric nerve where the lead will be implanted.

g) Cochlear Implants for the Treatment of Deafness

Neurostimulation is also used in the form of a cochlear implant that stimulates the auditory nerve for correcting sensorineural deafness. A sound processor captures sound from the environment and processes it into a digital signal that is transmitted via an antenna through the skin to the cochlear implant. The cochlear implant, which is surgically implanted in the cochlea adjacent to the auditory nerve, converts the digital information into electrical signals that are communicated to the auditory nerve via an electrode array. Effectively, the cochlear implant serves to bypass the nonfunctional cochlear transducers and directly depolarize afferent auditory nerve fibers. This stimulates the nerve to send signals to the auditory center in the brain and allows the patient to “hear” the sounds detected by the sound processor. The treatment is used for adults with 70 dB or greater hearing loss (and able to understand up to 50% of words in a sentence using a hearing aid) or children 12 months or older with 90 dB hearing loss in both ears.

Although many implantations are performed without incident, approximately 12-15% of patients experience some complications. Histologic assessment of cochlear implants has revealed that several forms of injury and scarring can occur. Surgical trauma can induce cochlear fibrosis, cochlear neossification and injury to the membranous cochlea (including loss of the sensorineural elements). A foreign body reaction along the implant and the electrode can produce a fibrous tissue response along the electrode array that has been associated with implant failure. Coating the implant and/or the electrode with an anti-scarring composition may help reduce the incidence of failure. As an alternative, or in addition to this, fibrosis may be reduced or prevented by the infiltration of an anti-scarring agent into the tissue (the scala tympani) where the electrodes contact the auditory nerve fibers.

A variety of suitable cochlear implant systems or “bionic ears” have been described for use in association with this invention. For example, the neurostimulator may be composed of a plurality of transducer elements which detect vibrations and then generates a stimulus signal to a corresponding neuron connected to the cranial nerve. See e.g., U.S. Pat. No. 5,061,282. The neurostimulator may be a cochlear implant having a sound-to-electrical stimulation encoder, a body implantable receiver-stimulator and electrodes, which emit pulses based on received electrical signals. See e.g., U.S. Pat. No. 4,532,930. The neurostimulator may be an intra-cochlear apparatus that is composed of a transducer that converts an audio signal into an electrical signal and an electrode array which electrically stimulates predetermined locations of the auditory nerve. See e.g., U.S. Pat. No. 4,400,590. The neurostimulator may be a stimulus generator for applying electrical stimuli to any branch of the 8^{th} nerve in a generally constant rate independent of audio modulation, such that it is perceived as active silence. See e.g., U.S. Pat. No. 6,175,767. The neurostimulator may be a subcranially implanted electromechanical system that has an input transducer and an output stimulator that converts a mechanical sound vibration into an electrical signal. See e.g., U.S. Pat. No. 6,235,056. The neurostimulator may be a cochlear implant that has a rechargeable battery housed within the implant for storing and providing electrical power. See e.g., U.S. Pat. No. 6,067,474. Other neurostimulators that are used as cochlear implants are described in, e.g., U.S. Pat. Nos. 6,358,281; 6,308,101 and 5,603,726.

Several commercially available devices are available for the treatment of patients with significant sensorineural hearing loss and are suitable for use with the present invention. For example, the HIRESOLUTION Bionic Ear System (Boston Scientific Corp., Nattick, Mass.) consists of the HIRES AURIA Processor which processes sound and sends a digital signal to the HIRES 90K Implant that has been surgically implanted in the inner ear. See e.g., U.S. Pat. Nos. 6,636,768; 6,309,410 and 6,259,951. The electrode array that transmits the impulses generated by the HIRES 90K Implant to the nerve may benefit from an anti-scarring coating and/or the infiltration of an anti-scarring agent into the region around the electrode-nerve interface. The PULSARci cochlear implant (MED-EL GMBH, Innsbruck, Austria, see e.g., U.S. Pat. Nos. 6,556,870 and 6,231,604) and the NUCLEUS 3 cochlear implant system (Cochlear Corp., Lane Cove, Australia, see e.g., U.S. Pat. Nos. 6,807,445; 6,788,790; 6,554,762; 6,537,200 and 6,394,947) are other commercial examples of cochlear implants whose electrodes are suitable for coating with an anti-scarring composition (or infiltration of an anti-scarring agent into the region around the electrode-nerve interface) under the present invention.

Regardless of the specific design features, for cochlear implants to be effective in sensorineural deafness, the electrode arrays must be accurately positioned adjacent to the afferent auditory nerve fibers. If excessive scar tissue growth or extracellular matrix deposition occurs around the leads, efficacy can be compromised. Cochlear implants that release a therapeutic agent able to reduce scarring at the electrode-tissue interface can increase the efficiency of impulse transmission and increase the duration that these devices function clinically. In one aspect, the device includes cochlear implants and/or leads that are coated with an anti-scarring agent or a composition that includes an anti-scarring agent. As an alternative to this, or in addition to this, a composition that includes an anti-scarring agent can be infiltrated into the cochlear tissue surrounding the lead.

h) Electrical Stimulation to Promote Bone Growth

In another aspect, electrical stimulation can be used to stimulate bone growth. For example, the stimulation device may be an electrode and generator having a strain response piezoelectric material which responds to strain by generating a charge to enhance the anchoring of an implanted bone prosthesis to the natural bone. See e.g., U.S. Pat. No. 6,143,035. If excessive scar tissue growth or extracellular matrix deposition occurs around the leads, efficacy can be compromised. Electrical bone stimulation devices that release a therapeutic agent able to reduce scarring at the electrode-tissue interface can increase the efficiency of impulse transmission and increase the duration that these devices function clinically. In one aspect, the device includes bone stimulation devices and/or leads that are coated with an anti-scarring agent or a composition that includes an anti-scarring agent. As an alternative to this, or in addition to this, a composition that includes an anti-scarring agent can be infiltrated into the bone tissue surrounding the electrical lead.

Although numerous neurostimulation devices have been described above, all possess similar design features and cause similar unwanted tissue reactions following implantation. It should be obvious to one of skill in the art that commercial neurostimulation devices not specifically sited above as well as next-generation and/or subsequently-developed commercial neurostimulation products are to be anticipated and are suitable for use under the present invention. The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices. Neurostimulator devices that release a therapeutic agent for reducing scarring (or gliosis) at the electrode-tissue interface can be used to increase the efficacy and/or the duration of activity of the implant (particularly for fully-implanted, battery-powered devices). In one aspect, the present invention provides neurostimulator devices that include an anti-scarring (or anti-gliosis) agent or a composition that includes an anti-scarring (or anti-gliosis) agent. Numerous polymeric and non-polymeric delivery systems for use in neurostimulator devices have been described above. These compositions can further include one or more fibrosis-inhibiting (or gliosis-inhibiting) agents such that the overgrowth of granulation, fibrous, or gliotic tissue is inhibited or reduced.

Methods for incorporating fibrosis-inhibiting (or gliosis-inhibiting) compositions onto or into these neurostimulator devices include: (a) directly affixing to the device, lead and/or the electrode a fibrosis-inhibiting (or gliosis-inhibiting) composition (e.g., by either a spraying process or dipping process as described above, with or without a carrier), (b) directly incorporating into the device, lead and/or the electrode a fibrosis-inhibiting (or gliosis-inhibiting) composition (e.g., by either a spraying process or dipping process as described above, with or without a carrier (c) by coating the device, lead and/or the electrode with a substance such as a hydrogel which may in turn absorb the fibrosis-inhibiting (or gliosis-inhibiting) composition, (d) by interweaving fibrosis-inhibiting (or gliosis-inhibiting) composition coated thread (or the polymer itself formed into a thread) into the device, lead and/or electrode structure, (e) by inserting the device, lead and/or the electrode into a sleeve or mesh which is comprised of, or coated with, a fibrosis-inhibiting (or gliosis-inhibiting) composition, (f) constructing the device, lead and/or the electrode itself (or a portion of the device and/or the electrode) with a fibrosis-inhibiting (or gliosis-inhibiting) composition, or (g) by covalently binding the fibrosis-inhibiting (or gliosis-inhibiting) agent directly to the device, lead and/or electrode surface or to a linker (small molecule or polymer) that is coated or attached to the device surface. Each of these methods illustrates an approach for combining an electrical device with a fibrosis-inhibiting (also referred to herein as an anti-scarring) or gliosis-inhibiting agent according to the present invention.

For these devices, leads and electrodes, the coating process can be performed in such a manner as to: (a) coat the non-electrode portions of the lead or device; (b) coat the electrode portion of the lead; or (c) coat all or parts of the entire device with the fibrosis-inhibiting (or gliosis-inhibiting) composition. In addition to, or alternatively, the fibrosis-inhibiting (or gliosis-inhibiting) agent can be mixed with the materials that are used to make the device, lead and/or electrode such that the fibrosis-inhibiting agent is incorporated into the final product. In these manners, a medical device may be prepared which has a coating, where the coating is, e.g., uniform, non-uniform, continuous, discontinuous, or patterned.

In another aspect, a neurostimulation device may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void. Thus, the coating of the medical device may directly contact the electrical device, or it may indirectly contact the electrical device when there is something, e.g., a polymer layer, that is interposed between the electrical device and the coating that contains the fibrosis-inhibiting agent.

In addition to, or as an alternative to incorporating a fibrosis-inhibiting (or gliosis-inhibiting) agent onto or into the neurostimulation device, the fibrosis-inhibiting (or gliosis-inhibiting) agent can be applied directly or indirectly to the tissue adjacent to the neurostimulator device (preferably near the electrode-tissue interface). This can be accomplished by applying the fibrosis-inhibiting (or gliosis inhibiting) agent, with or without a polymeric, non-polymeric, or secondary carrier: (a) to the lead and/or electrode surface (e.g., as an injectable, paste, gel or mesh) during the implantation procedure); (b) to the surface of the tissue (e.g., as an injectable, paste, gel, in situ forming gel or mesh) prior to, immediately prior to, or during, implantation of the neurostimulation device, lead and/or electrode; (c) to the surface of the lead and/or electrode and/or the tissue surrounding the implanted lead and/or electrode (e.g., as an injectable, paste, gel, in situ forming gel or mesh) immediately after to the implantation of the neurostimulation device, lead and/or electrode; (d) by topical application of the anti-fibrosis (or gliosis) agent into the anatomical space where the neurostimulation device, lead and/or electrode will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the device, lead and/or electrode as a solution as an infusate or as a sustained release preparation; (f) by any combination of the aforementioned methods. Combination therapies (i.e., combinations of therapeutic agents and combinations with antithrombotic and/or antiplatelet agents) can also be used.

It should be noted that certain polymeric carriers themselves can help prevent the formation of fibrous or gliotic tissue around the neuroimplant. These carriers (to be described shortly) are particularly useful for the practice of this embodiment, either alone, or in combination with a fibrosis (or gliosis) inhibiting composition. The following polymeric carriers can be infiltrated (as described in the previous paragraph) into the vicinity of the electrode-tissue interface and include: (a) sprayable collagen-containing formulations such as COSTASIS and crosslinked derivatized poly(ethylene glycol)-collagen compositions (described, e.g., in U.S. Pat. Nos. 5,874,500 and 5,565,519 and referred to herein as “CT3” (both from Angiotech Pharmaceuticals, Inc., Canada), either alone, or loaded with a fibrosis-inhibiting (or gliosis-inhibiting) agent, applied to the implantation site (or the implant/device surface); (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Conf.luent Surgical, Inc., Boston, Mass.), either alone, or loaded with a fibrosis-inhibiting (or gliosis-inhibiting) agent, applied to the implantation site (or the implant/device surface); (c) fibrinogen-containing formulations such as FLOSEAL or TISSEAL (both from Baxter Healthcare Corporation, Fremont, Calif.), either alone, or loaded with a fibrosis-inhibiting (or gliosis-inhibiting) agent, applied to the implantation site (or the implant/device surface); (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), loaded with a fibrosis-inhibiting (or gliosis-inhibiting) agent applied to the implantation site (or the implant/device surface); (e) polymeric gels for surgical implantation such as REPEL (Life Medical Sciences, Inc., Princeton, N.J.) or FLOWGEL (Baxter Healthcare Corporation) loaded with a fibrosis-inhibiting (or gliosis-inhibiting) agent applied to the implantation site (or the implant/device surface); (f) orthopedic “cements” used to hold prostheses and tissues in place loaded with a fibrosis-inhibiting (or gliosis-inhibiting) agent applied to the implantation site (or the implant/device surface), such as OSTEOBOND (Zimmer, Inc., Warsaw, Ind.), low viscosity cement (LVC); Wright Medical Technology, Inc., Arlington, Tenn.), SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc.), INDERMIL (U.S. Surgical Company, Norwalk, Conn.), GLUSTITCH (Blacklock Medical Products Inc., Canada), TISSUEMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.), either alone, or loaded with a fibrosis-inhibiting (or gliosis-inhibiting) agent, applied to the implantation site (or the implant/device surface); (h) implants containing hydroxyapatite [or synthetic bone material such as calcium sulfate, VITOSS and CORTOSS (both from Orthovita, Inc., Malvern, Pa.) loaded with a fibrosis-inhibiting (or gliosis-inhibiting) agent applied to the implantation site (or the implant/device surface); (i) other biocompatible tissue fillers loaded with a fibrosis-inhibiting (or gliosis-inhibiting) agent, such as those made by BioCure, Inc. (Norcross, Ga.), 3M Company (St. Paul, Minn.) and Neomend, Inc. (Sunnyvale, Calif.), applied to the implantation site (or the implant/device surface); (j) polysaccharide gels such as the ADCON series of gels (available from Gliatech, Inc., Cleveland, Ohio) either alone, or loaded with a fibrosis-inhibiting (or gliosis-inhibiting) agent, applied to the implantation site (or the implant/device surface); and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) loaded with a fibrosis-inhibiting (or gliosis-inhibiting) agent applied to the implantation site (or the implant/device surface).

A preferred polymeric matrix which can be used to help prevent the formation of fibrous or gliotic tissue around the neuroimplant, either alone or in combination with a fibrosis (or gliosis) inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous or gliotic tissue around the neuroimplant.

It should be apparent to one of skill in the art that potentially any anti-scarring (or anti-gliotic) agent described above may be utilized alone, or in combination, in the practice of this embodiment. As neurostimulator devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the device (i.e., as a coating or infiltrated into the surrounding tissue), the fibrosis-inhibiting (or gliosis-inhibiting) agents, used alone or in combination, may be administered under the following dosing guidelines:

Drugs and dosage: Exemplary therapeutic agents that may be used include, but are not limited to: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

Provided below are exemplary dosage ranges for various anti-scarring agents that can be used in conjunction with compositions for treating or preventing surgical adhesions in accordance with the invention. (A) Angiogenesis inhibitors including alphastatin, ZD-6474, IDN-5390, SB-2723005, ABT-518, combretastatin, and anecortane, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (B) mTOR inhibitors including AP-23573 and temsirolimus, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (C) Tubulin antagonists including synthadotin, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (D) Epithilones including ixabepilone and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (E) Kinesin Antagonists including SB-715992 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (F) TNF alpha antagonists including etanercept, humicade, adalimumab and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (G) AKT inhibitor including erucylphosphocholine and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (H) FGF Inhibitors including IDN-5390 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (I) Collagenase Antagonists including S-0885 and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface. (J) NF KAPPA B Inhibitors including bortezomib and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (K) Elongation Factor-1 alpha inhibitors including aplidine and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface. (L) Tyrosine kinase inhibitors including gefitinib and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface.

Additional examples of anti-scarring agents which can be used include those having a high potency in the assays described herein (approximately 1-100 nM IC₅₀ range) such as isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, and tacrolimus. For high potency drugs, the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁻⁴ M of agent should be maintained on the implant or barrier surface.

Other examples of agents which can be used include those having a mid-potency in the assays described herein (approximately 100-500 nM IC₅₀ range) such as loteprednol etabonate, juglone, prednisolone, puromycin, 3-BAABE, cladribine, and mannose-6-phosphate. For mid-potency drugs, the total dose typically should not to exceed 500 mg (range of 1.0 μg to 500 mg) and preferably 1 μg to 200 mg; dose per unit area of 0.01 μg-200 μg per mm², preferably 0.1 μg/mm²-40 μg/mm²; and minimum concentration of 10⁻⁸-10⁻⁴ M of agent should be maintained on the implant or barrier surface.

Other examples of agents which can be used include those having a low potency in the assays described herein (approximately 500-1000 nm range IC₅₀ range) such as 5-azacytidine, Ly333531 (ruboxistaurin), and simvastatin. For low potency drugs, the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm²; and minimum concentration of 10⁻⁸-10⁻⁴ M of agent should to be maintained on the implant or barrier surface.

2) Cardiac Rhythm Management (CRM) Devices

In another aspect, the electrical device may be a cardiac pacemaker device where a pulse generator delivers an electrical impulse to myocardial tissue (often specialized conduction fibres) via an implanted lead in order to regulate cardiac rhythm. Typically, electrical leads are composed of a connector assembly, a lead body (i.e., conductor) and an electrode. Electrical leads may be unipolar, in which they are adapted to provide effective therapy with only one electrode. Multi-polar leads are also available, including bipolar, tripolar and quadripolar leads. Electrical leads may also have insulating sheaths which may include polyurethane or silicone-rubber coatings. Representative examples of electrical leads include, without limitation, medical leads, cardiac leads, pacer leads, pacing leads, pacemaker leads, endocardial leads, endocardial pacing leads, cardioversion/defibrillator leads, cardioversion leads, epicardial leads, epicardial defibrillator leads, patch defibrillators, patch leads, electrical patch, transvenous leads, active fixation leads, passive fixation leads and sensing leads Representative examples of CRM devices that utilize electrical leads include: pacemakers, LVAD's, defibrillators, implantable sensors and other electrical cardiac stimulation devices.

There are numerous pacemaker devices where the occurrence of a fibrotic reaction will adversely affect the functioning of the device or cause damage to the myocardial tissue. Typically, fibrotic encapsulation of the pacemaker lead (or the growth of fibrous tissue between the lead and the target myocardial tissue) slows, impairs, or interrupts electrical transmission of the impulse from the device to the myocardium. For example, fibrosis is often found at the electrode-myocardial interfaces in the heart, which may be attributed to electrical injury from focal points on the electrical lead. The fibrotic injury may extend into the tricuspid valve, which may lead to perforation. Fibrosis may lead to thrombosis of the subclavian vein; a condition which may be life-threatening. Electrical leads that release therapeutic agent for reducing scarring at the electrode-tissue interface may help prolong the clinical performance of these devices. Not only can fibrosis cause the device to function suboptimally or not at all, it can cause excessive drain on battery life as increased energy is required to overcome the electrical resistance imposed by the intervening scar tissue. Similarly, fibrotic encapsulation of the sensing components of a rate-responsive pacemaker (described below) can impair the ability of the pacemaker to identify and correct rhythm abnormalities leading to inappropriate pacing of the heart or the failure to function correctly when required.

Several different electrical pacing devices are used in the treatment of various cardiac rhythm abnormalities including pacemakers, implantable cardioverter defibrillators (ICD), left ventricular assist devices (LVAD), and vagus nerve stimulators (stimulates the fibers of the vagus nerve which in turn innervate the heart). The pulse generating portion of device sends electrical impulses via implanted leads to the muscle (myocardium) or conduction tissue of the heart to affect cardiac rhythm or contraction. Pacing can be directed to one or more chambers of the heart. Cardiac pacemakers may be used to block, mask, or stimulate electrical signals in the heart to treat dysfunctions, including, without limitation, atrial rhythm abnormalities, conduction abnormalities and ventricular rhythm abnormalities. ICDs are used to depolarize the ventricals and re-establish rhythm if a ventricular arrhythmia occurs (such as asystole or ventricular tachycardia) and LVADs are used to assist ventricular contraction in a failing heart.

Representative examples of patents which describe pacemakers and pacemaker leads include U.S. Pat. Nos. 4,662,382, 4,782,836, 4,856,521, 4,860,751, 5,101,824, 5,261,419, 5,284,491, 6,055,454, 6,370,434, and 6,370,434. Representative examples of electrical leads include those found on a variety of cardiac devices, such as cardiac stimulators (see e.g., U.S. Pat. Nos. 6,584,351 and 6,115,633), pacemakers (see e.g., U.S. Pat. Nos. 6,564,099; 6,246,909 and 5,876,423), implantable cardioverter-defibrillators (ICDs), other defibrillator devices (see e.g., U.S. Pat. No. 6,327,499), defibrillator or demand pacer catheters (see e.g., U.S. Pat. No. 5,476,502) and Left Ventricular Assist Devices (see e.g., U.S. Pat. No. 5,503,615).

Cardiac rhythm devices, and in particular the lead(s) that deliver the electrical pulsation, must be positioned in a very precise manner to ensure that stimulation is delivered to the correct anatomical location in the heart. All, or parts, of a pacing device can migrate following surgery, or excessive scar tissue growth can occur around the lead, which can lead to a reduction in the performance of these devices (as described previously). Cardiac rhythm management devices that release a therapeutic agent for reducing scarring at the electrode-tissue interface can be used to increase the efficacy and/or the duration of activity (particularly for fully-implanted, battery-powered devices) of the implant. Accordingly, the present invention provides cardiac leads that are coated with an anti-scarring agent or a composition that includes an anti-scarring agent.

For greater clarity, several specific cardiac rhythm management devices and treatments will be described in greater detail including:

a) Cardiac Pacemakers

Cardiac rhythm abnormalities are extremely common in clinical practice and the incidence increases in frequency with both age and the presence of underlying coronary artery disease or myocardial infarction. A litany of arrythmias exists, but they are generally categorized into conditions where the heart beats too slowly (bradyarrythmias—such heart block, sinus node dysfunction) or too quickly (tachyarrhythmias—such as atrial fibrillation, WPW syndrome, ventricular fibrillation). A pacemaker functions by sending an electrical pulse (a pacing pulse) that travels via an electrical lead to the electrode (at the tip of the lead) which delivers an electrical impulse to the heart that initiates a heartbeat. The leads and electrodes can be located in one chamber (either the right atrium or the right ventricle—called single-chamber pacemakers) or there can be electrodes in both the right atrium and the right ventricle (called dual-chamber pacemakers). Electrical leads may be implanted on the exterior of the heart (e.g., epicardial leads) by a surgical procedure, or they can be connected to the endocardial surface of the heart via a catheter, guidewire or stylet. In some pacemakers, the device assumes the rhythm generating function of the heart and fires at a regular rate. In other pacemakers, the device merely augments the heart's own pacing function and acts “on demand” to provide pacing assistance as required (called “adaptive-rate” pacemakers); the pacemaker receives feedback on heart rhythm (and hence when to fire) from an electrode sensor located on the lead. Other pacemakers, called rate responsive pacemakers, have special sensors that detect changes in body activity (such as movement of the arms and legs, respiratory rate) and adjust pacing up or down accordingly.

Numerous pacemakers and pacemaker leads are suitable for use in this invention. For example, the pacing lead may have an increased resistance to fracture by being composed of an elongated coiled conductor mounted within a lumen of a lead body whereby it may be coupled electrically to a stranded conductor. See e.g., U.S. Pat. Nos. 6,061,598 and 6,018,683. The pacing lead may have a coiled conductor with an insulated sheath, which has a resistance to crush fatigue in the region between the rib and clavicle. See e.g., U.S. Pat. No. 5,800,496. The pacing lead may be expandable from a first, shorter configuration to a second, longer configuration by being composed of slideable inner and outer overlapping tubes containing a conductor. See e.g., U.S. Pat. No. 5,897,585. The pacing lead may have the means for temporarily making the first portion of the lead body stiffer by using a magnet-rheologic fluid in a cavity that stiffens when exposed to a magnetic field. See e.g., U.S. Pat. No. 5,800,497. The pacing lead may be a coil configuration composed of a plurality of wires or wire bundles made from a duplex titanium alloy. See e.g., U.S. Pat. No. 5,423,881. The pacing lead may be composed of a wire wound in a coil configuration with the wire composed of stainless steel having a composition of at least 22% nickel and 2% molybdenum. See e.g., U.S. Pat. No. 5,433,744. Other pacing leads are described in, e.g., U.S. Pat. Nos. 6,489,562; 6,289,251 and 5,957,967.

In another aspect, the electrical lead used in the practice of this invention may have an active fixation element for attachment to tissue. For example, the electrical lead may have a rigid fixation helix with microgrooves that are dimensioned to minimize the foreign body response following implantation. See e.g., U.S. Pat. No. 6,078,840. The electrical lead may have an electrode/anchoring portion with a dual tapered self-propelling spiral electrode for attachment to vessel wall. See e.g., U.S. Pat. No. 5,871,531. The electrical lead may have a rigid insulative electrode head carrying a helical electrode. See e.g., U.S. Pat. No. 6,038,463. The electrical lead may have an improved anchoring sleeve designed with an introducer sheath to minimize the flow of blood through the sheath during introduction. See e.g., U.S. Pat. No. 5,827,296. The electrical lead may be composed of an insulated electrical conductive portion and a lead-in securing section having a longitudinally rigid helical member which may be screwed into tissue. See e.g., U.S. Pat. No. 4,000,745.

Suitable leads for use in the practice of this invention also include multi-polar leads with multiple electrodes connected to the lead body. For example, the electrical lead may be a multi-electrode lead whereby the lead has two internal conductors and three electrodes with two electrodes coupled by a capacitor integral with the lead. See e.g., U.S. Pat. No. 5,824,029. The electrical lead may be a lead body with two straight sections and a bent third section with associated conductors and electrodes whereby the electrodes are bipolar. See e.g., U.S. Pat. No. 5,995,876. In another aspect, the electrical lead may be implanted by using a catheter, guidewire or stylet. For example, the electrical lead may be composed of an elongated insulative lead body having a lumen with a conductor mounted within the lead body and a resilient seal having an expandable portion through which a guidewire may pass. See e.g., U.S. Pat. No. 6,192,280.

Commercially available pacemakers suitable for the practice of the invention include the KAPPA SR 400 Series single-chamber rate-responsive pacemaker system, the KAPPA DR 400 Series dual-chamber rate-responsive pacemaker system, the KAPPA 900 and 700 Series single-chamber rate-responsive pacemaker system, and the KAPPA 900 and 700 Series dual-chamber rate-responsive pacemaker system by Medtronic, Inc. Medtronic pacemaker systems utilize a variety leads including the CAPSURE Z Novus, CAPSUREFIX Novus, CAPSUREFIX, CAPSURE SP Novus, CAPSURE SP, CAPSURE EPI and the CAPSURE VDD which may be suitable for coating with a fibrosis-inhibiting agent. Pacemaker systems and associated leads that are made by Medtronic are described in, e.g., U.S. Pat. Nos. 6,741,893; 5,480,441; 5,411,545; 5,324,310; 5,265,602; 5,265,601; 5,241,957 and 5,222,506 Medtronic also makes a variety of steroid-eluting leads including those described in, e.g., U.S. Pat. Nos. 5,987,746; 6,363,287; 5,800,470; 5,489,294; 5,282,844 and 5,092,332. The INSIGNIA single-chamber and dual-chamber system, PULSAR MAX II DR dual-chamber adaptive-rate pacemaker, PULSAR MAX II SR single-chamber adaptive-rate pacemaker, DISCOVERY II DR dual-chamber adaptive-rate pacemaker, DISCOVERY II SR single-chamber adaptive-rate pacemaker, DISCOVERY II DDD dual-chamber pacemaker, and the DISCOVERY II SSI dingle-chamber pacemaker systems made by Guidant Corp. (Indianapolis, Ind.) are also suitable pacemaker systems for the practice of this invention. Once again, the leads from the Guidant pacemaker systems may be suitable for coating with a fibrosis-inhibiting agent. Pacemaker systems and associated leads that are made by Guidant are described in, e.g., U.S. Pat. Nos. 6,473,648; 6,345,204; 6,321,122; 6,152,954; 5,769,881; 5,284,136; 5,086,773 and 5,036,849. The AFFINITY DR, AFFINITY VDR, AFFINITY SR, AFFINITY DC, ENTITY, IDENTITY, IDENTITY ADX, INTEGRITY, INTEGRITY μDR, INTEGRITY ADx, MICRONY, REGENCY, TRILOGY, and VERITY ADx, pacemaker systems and leads from St. Jude Medical, Inc. (St. Paul, Minn.) may also be suitable for use with a fibrosis-inhibiting coating to improve electrical transmission and sensing by the pacemaker leads. Pacemaker systems and associated leads that are made by St. Jude Medical are described in, e.g., U.S. Pat. Nos. 6,763,266; 6,760,619; 6,535,762; 6,246,909; 6,198,973; 6,183,305; 5,800,468 and 5,716,390. Alternatively, the fibrosis-inhibiting agent may be infiltrated into the region around the electrode-cardiac muscle interface under the present invention. It should be obvious to one of skill in the art that commercial pacemakers not specifically sited as well as next-generation and/or subsequently developed commercial pacemaker products are to be anticipated and are suitable for use under the present invention.

Regardless of the specific design features, for pacemakers to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. Pacemaker leads that release a therapeutic agent able to reduce scarring at the electrode-tissue and/or sensor-tissue interface, can increase the efficiency of impulse transmission and rhythm sensing, thereby increasing efficacy and battery longevity. In one aspect, the device includes pacemaker leads that are coated with an anti-scarring agent or a composition that includes an anti-scarring agent. As an alternative to this, or in addition to this, a composition that includes an anti-scarring agent can be infiltrated into the myocardial tissue surrounding the lead.

b) Implantable Cardioverter Defibrillator (ICD) Systems

Implantable cardioverter defibrillator (ICD) systems are similar to pacemakers (and many include a pacemaker system), but are used for the treatment of tachyarrhythmias such as ventricular tachycardia or ventricular fibrillation. An ICD consists of a mini-computer powered by a battery which is connected to a capacitor to helps the ICD charge and store enough energy to deliver therapy when needed. The ICD uses sensors to monitor the activity of the heart and the computer analysizes the data to determine when and if an arrhythmia is present. An ICD lead, which is inserted via a vein (called “transvenous” leads; in some systems the lead is implanted surgically—called an epicardial lead—and sewn onto the surface of the heart), connects into the pacing/computer unit. The lead, which is usually placed in the right ventricle, consists of an insulated wire and an electrode tip that contains a sensing component (to detect cardiac rhythm) and a shocking coil. A single-chamber ICD has one lead placed in the ventricle which defibrillates and paces the ventricle, while a dual-chamber ICD defibrillates the ventricle and paces the atrium and the ventricle. In some cases, an additional lead is required and is placed under the skin next to the rib cage or on the surface of the heart. In patients who require tachyarrhythmia management of the ventricle and atrium, a second coil is placed in the atrium to treat atrial tachycardia, atrial fibrillation and other arrhythmias. If a tachyarrhythmia is detected, a pulse is generated and propagated via the lead to the shocking coil which delivers a charge sufficient to depolarize the muscle and cardiovert or defibrillate the heart.

Several ICD systems have been described and are suitable for use in the practice of this invention. Representative examples of ICD's and associated components are described in U.S. Pat. Nos. 3,614,954, 3,614,955, 4,375,817, 5,314,430, 5,405,363, 5,607,385, 5,697,953, 5,776,165, 6,067,471, 6,169,923, and 6,152,955. Several ICD leads are suitable for use in the practice of this invention. For example, the defibrillator lead may be a linear assembly of sensors and coils formed into a loop which includes a conductor system for coupling the loop system to a pulse generator. See e.g., U.S. Pat. No. 5,897,586. The defibrillator lead may have an elongated lead body with an elongated electrode extending from the lead body, such that insulative tubular sheaths are slideably mounted around the electrode. See e.g., U.S. Pat. No. 5,919,222. The defibrillator lead may be a temporary lead with a mounting pad and a temporarily attached conductor with an insulative sleeve whereby a plurality of wire electrodes are mounted. See e.g., U.S. Pat. No. 5,849,033. Other defibrillator leads are described in, e.g., U.S. Pat. No. 6,052,625. In another aspect, the electrical lead may be adapted to be used for pacing, defibrillating or both applications. For example, the electrical lead may be an electrically insulated, elongated, lead body sheath enclosing a plurality of lead conductors that are separated from contacting one another. See e.g., U.S. Pat. No. 6,434,430. The electrical lead may be composed of an inner lumen adapted to receive a stiffening member (e.g., guide wire) that delivers fluoro-visible media. See e.g., U.S. Pat. No. 6,567,704. The electrical lead may be a catheter composed of an elongated, flexible, electrically nonconductive probe contained within an electrically conductive pathway that transmits electrical signals, including a defibrillation pulse and a pacer pulse, depending on the need that is sensed by a governing element. See e.g., U.S. Pat. No. 5,476,502. The electrical lead may have a low electrical resistance and good mechanical resistance to cyclical stresses by being composed of a conductive wire core formed into a helical coil covered by a layer of electrically conductive material and an electrically insulating sheath covering. See e.g., U.S. Pat. No. 5,330,521. Other electrical leads that may be adapted for use in pacing and/or defibrillating applications are described in, e.g., U.S. Pat. No. 6,556,873.

Commercially available ICDs suitable for the practice of the invention include the GEM III DR dual-chamber ICD, GEM III VR ICD, GEM II ICD, GEM ICD, GEM III AT atrial and ventricular arrhythmia ICD, JEWEL AF dual-chamber ICD, MICRO JEWEL ICD, MICRO JEWEL II ICD, JEWEL Plus ICD, JEWEL ICD, JEWEL ACTIVE CAN ICD, JEWEL PLUS ACTIVE CAN ICD, MAXIMO DR ICD, MAXIMO VR ICD, MARQUIS DR ICD, MARQUIS VR system, and the INTRINSIC dual-chamber ICD by Medtronic, Inc. Medtronic ICD systems utilize a variety leads including the SPRINT FIDELIS, SPRINT QUATRO SECURE steroid-eluting bipolar lead. Subcutaneous Lead System Model 6996SQ subcutaneous lead, TRANSVENE 6937A transvenous lead, and the 6492 Unipolar Atrial Pacing Lead which may be suitable for coating with a fibrosis-inhibiting agent. ICD systems and associated leads that are made by Medtronic are described in, e.g., U.S. Pat. Nos. 6,038,472; 5,849,031; 5,439,484; 5,314,430; 5,165,403; 5,099,838 and 4,708,145. The VITALITY 2 DR dual-chamber ICD, VITALITY 2 VR single-chamber ICD, VITALITY AVT dual-chamber ICD, VITALITY DS dual-chamber ICD, VITALITY DS VR single-chamber ICD, VITALITY EL dual-chamber ICD, VENTAK PRIZM 2 DR dual-chamber ICD, and VENTAK PRIZM 2 VR single-chamber ICD systems made by Guidant Corp. are also suitable ICD systems for the practice of this invention Once again, the leads from the Guidant ICD systems may be suitable for coating with a fibrosis-inhibiting agent. Guidant sells the FLEXTEND Bipolar Leads, EASYTRAK Lead System, FINELINE Leads, and ENDOTAK RELIANCE ICD Leads. ICD systems and associated leads that are made by Guidant are described in, e.g., U.S. Pat. Nos. 6,574,505; 6,018,681; 5,697,954; 5,620,451; 5,433,729; 5,350,404; 5,342,407; 5,304,139 and 5,282,837. Biotronik, Inc. (Germany) sells the POLYROX Endocardial Leads, KENTROX SL Quadripolar ICD Leads, AROX Bipolar Leads, and MAPOX Bipolar Epicardial Leads (see e.g., U.S. Pat. Nos. 6,449,506; 6,421,567; 6,418,348; 6,236,893 and 5,632,770). The CONTOUR MD ICD, PHOTON μ DR ICD, PHOTON μ VR ICD, ATLAS+ HF ICD, EPIC HF ICD, EPIC+ HF ICD systems and leads from St. Jude Medical may also be suitable for use with a fibrosis-inhibiting coating to improve electrical transmission and sensing by the ICD leads (see e.g., U.S. Pat. Nos. 5,944,746; 5,722,994; 5,662,697; 5,542,173; 5,456,706 and 5,330,523). Alternatively, the fibrosis-inhibiting agent may be infiltrated into the region around the electrode-cardiac muscle interface under the present invention. It should be obvious to one of skill in the art that commercial ICDs not specifically sited as well as next-generation and/or subsequently developed commercial ICD products are to be anticipated and are suitable for use under the present invention.

Regardless of the specific design features, for ICDs to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. ICD leads that release a therapeutic agent able to reduce scarring at the electrode-tissue and/or sensor-tissue interface, can increase the efficiency of impulse transmission and rhythm sensing, thereby increasing efficacy, preventing inappropriate cardioversion, and improving battery longevity. In one aspect, the device includes ICD leads that are coated with an anti-scarring agent or a composition that includes an anti-scarring agent. As an alternative to this, or in addition to this, a composition that includes an anti-scarring agent can be infiltrated into the myocardial tissue surrounding the lead.

c) Vagus Nerve stimulation for the Treatment of Arrhythmia

In another aspect, a neurostimulation device may be used to stimulate the vagus nerve and affect the rhythm of the heart. Since the vagus nerve provides innervation to the heart, including the conduction system (including the SA node), stimulation of the vagus nerve may be used to treat conditions such as supraventricular arrhythmias, angina pectoris, atrial tachycardia, atrial flutter, atrial fibrillation and other arrhythmias that result in low cardiac output.

As described above, in VNS a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the cardiologist to treat a specific arrhythmia.

Products such as these have been described, for example, in U.S. Pat. Nos. 6,597,953 and 6,615,085. For example, the neurostimulator may be a vagal-stimulation apparatus which generates pulses at a frequency that varies automatically based on the excitation rates of the vagus nerve. See e.g., U.S. Pat. Nos. 5,916,239 and 5,690,681. The neurostimulator may be an apparatus that detects characteristics of tachycardia based on an electrogram and delivers a preset electrical stimulation to the nervous system to depress the heart rate. See e.g., U.S. Pat. No. 5,330,507. The neurostimulator may be an implantable heart stimulation system composed of two sensors, one for atrial signals and one for ventricular signals, and a pulse generator and control unit, to ensure sympatho-vagal stimulation balance. See e.g., U.S. Pat. No. 6,477,418. The neurostimulator may be a device that applies electrical pulses to the vagus nerve at a programmable frequency that is adjusted to maintain a lower heart rate. See e.g., U.S. Pat. No. 6,473,644. The neurostimulator may provide electrical stimulation to the vagus nerve to induce changes to electroencephalogram readings as a treatment for epilepsy, while controlling the operation of the heart within normal parameters. See e.g., U.S. Pat. No. 6,587,727.

A commercial example of a VNS system is the product produced by Cyberonics Inc. that consists of the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,928,272; 5,540,730 and 5,299,569.

Regardless of the specific design features, for vagal nerve stimulation to be effective in arrhythmias, the leads must be accurately positioned adjacent to the left vagus nerve. If excessive scar tissue growth or extracellular matrix deposition occurs around the VNS leads, this can reduce the efficacy of the device. VNS devices that release a therapeutic agent able to reducing scarring at the electrode-tissue interface can increase the efficiency of impulse transmission and increase the duration that these devices function clinically. In one aspect, the device includes VNS devices and/or leads that are coated with an anti-scarring agent or a composition that includes an anti-scarring agent. As an alternative to this, or in addition to this, a composition that includes an anti-scarring agent can be infiltrated into the tissue surrounding the vagus nerve where the lead will be implanted.

Although numerous cardiac rhythm management (CRM) devices have been described above, all possess similar design features and cause similar unwanted fibrous tissue reactions following implantation. The CRM device, particularly the lead(s), must be positioned in a very precise manner to ensure that stimulation is delivered to the correct anatomical location within the atrium and/or ventricle. All, or parts, of a CRM device can migrate following surgery, or excessive scar tissue growth can occur around the implant, which can lead to a reduction in the performance of these devices. CRM devices that release a therapeutic agent for reducing scarring at the electrode-tissue interface can be used to increase the efficacy and/or the duration of activity of the implant (particularly for fully-implanted, battery-powered devices). In one aspect, the present invention provides CRM devices that include a fibrosis-inhibiting agent or a composition that includes a fibrosis-inhibiting agent. Numerous polymeric and non-polymeric delivery systems for use in CRM devices have been described above. These compositions can further include one or more fibrosis-inhibiting agents such that the overgrowth of granulation or fibrous tissue is inhibited or reduced.

Methods for incorporating fibrosis-inhibiting compositions onto or into CRM devices include: (a) directly affixing to the CRM device, lead and/or electrode a fibrosis-inhibiting composition (e.g., by either a spraying process or dipping process as described above, with or without a carrier), (b) directly incorporating into the CRM device, lead and/or electrode a fibrosis-inhibiting composition (e.g., by either a spraying process or dipping process as described above, with or without a carrier (c) by coating the CRM device, lead and/or electrode with a substance such as a hydrogel which will in turn absorb the fibrosis-inhibiting composition, (d) by interweaving fibrosis-inhibiting composition coated thread (or the polymer itself formed into a thread) into the device, lead and/or electrode structure, (e) by inserting the CRM device, lead and/or electrode into a sleeve or mesh which is comprised of, or coated with, a fibrosis-inhibiting composition, (f) constructing the CRM device, lead and/or electrode itself (or a portion of the lead and/or electrode) with a fibrosis-inhibiting composition, or (g) by covalently binding the fibrosis-inhibiting agent directly to the CRM device, lead and/or electrode surface, or to a linker (small molecule or polymer) that is coated or attached to the device, lead and/or electrode surface. Each of these methods illustrates an approach for combining an electrical device with a fibrosis-inhibiting (also referred to herein as an anti-scarring) or gliosis-inhibiting agent according to the present invention.

For CRM devices, leads and electrodes, the coating process can be performed in such a manner as to: (a) coat the non-electrode portions of the lead; (b) coat the electrode portion of the lead; or (c) coat all or parts of the entire device with the fibrosis-inhibiting composition. In addition to, or alternatively, the fibrosis-inhibiting agent can be mixed with the materials that are used to make the CRM device, lead and/or electrode such that the fibrosis-inhibiting agent is incorporated into the final product. In these manners, a medical device may be prepared which has a coating, where the coating is, e.g., uniform, non-uniform, continuous, discontinuous, or patterned.

In another aspect, a CRM device may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void. Thus, the coating of the medical device may directly contact the electrical device, or it may indirectly contact the electrical device when there is something, e.g., a polymer layer, that is interposed between the electrical device and the coating that contains the fibrosis-inhibiting agent.

In addition to, or as an alternative to incorporating a fibrosis-inhibiting agent onto, or into, the CRM device, the fibrosis-inhibiting agent can be applied directly or indirectly to the tissue adjacent to the CRM device (preferably near the electrode-tissue interface). This can be accomplished by applying the fibrosis-inhibiting agent, with or without a polymeric, non-polymeric, or secondary carrier: (a) to the lead and/or electrode surface (e.g., as an injectable, paste, gel, or mesh) during the implantation procedure; (b) to the surface of the tissue (e.g., as an injectable, paste, gel, in situ forming gel, or mesh) prior to, immediately prior to, or during, implantation of the CRM device and/or the lead; (c) to the surface of the CRM lead and/or electrode and/or to the tissue surrounding the implanted lead or electrode (e.g., as an injectable, paste, gel, in situ forming gel, or mesh) immediately after the implantation of the CRM device, lead and/or electrode; (d) by topical application of the anti-fibrosis agent into the anatomical space where the CRM device, lead and/or electrode will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the CRM device, lead and/or electrode will be inserted); (e) via percutaneous injection into the tissue surrounding the CRM device, lead and/or electrode as a solution, as an infusate, or as a sustained release preparation; (f) by any combination of the aforementioned methods. Combination therapies (i.e., combinations of therapeutic agents and combinations with antithrombotic and/or antiplatelet agents) can also be used.

It should be noted that certain polymeric carriers themselves can help prevent the formation of fibrous tissue around the CRM lead and electrode. These carriers (to be described shortly) are particularly useful for the practice of this embodiment, either alone, or in combination with a fibrosis-inhibiting composition. The following polymeric carriers can be infiltrated (as described in the previous paragraph) into the vicinity of the CRM device, lead and/or electrode-tissue interface and include: (a) sprayable collagen-containing formulations such as COSTASIS and CT3, either alone, or loaded with a fibrosis-inhibiting agent, applied to the implantation site (or the implant/device surface); (b) sprayable PEG-containing formulations such as COSEAL, FOCALSEAL, SPRAYGEL or DURASEAL, either alone, or loaded with a fibrosis-inhibiting agent, applied to the implantation site (or the implant/device surface); (c) fibrinogen-containing formulations such as FLOSEAL or TISSEAL, either alone, or loaded with a fibrosis-inhibiting agent, applied to the implantation site (or the implant/device surface); (d) hyaluronic acid-containing formulations such as RESTYLANE, HYLAFORM, PERLANE, SYNVISC, SEPRAFILM, SEPRACOAT, loaded with a fibrosis-inhibiting agent applied to the implantation site (or the implant/device surface); (e) polymeric gels for surgical implantation such as REPEL or FLOWGEL loaded with a fibrosis-inhibiting agent applied to the implantation site (or the implant/device surface); (f) orthopedic “cements” used to hold prostheses and tissues in place loaded with a fibrosis-inhibiting agent applied to the implantation site (or the implant/device surface), such as OSTEOBOND, low viscosity cement (LVC), SIMPLEX P, PALACOS, and ENDURANCE; (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT, either alone, or loaded with a fibrosis-inhibiting agent, applied to the implantation site (or the implant/device surface); (h) implants containing hydroxyapatite [or synthetic bone material such as calcium sulfate, VITOSS and CORTOSS (Orthovita)] loaded with a fibrosis-inhibiting agent applied to the implantation site (or the implant/device surface); (i) other biocompatible tissue fillers loaded with a fibrosis-inhibiting agent, such as those made by BioCure, Inc., 3M Company and Neomend, Inc., applied to the implantation site (or the implant/device surface); (j) polysaccharide gels such as the ADCON series of gels either alone, or loaded with a fibrosis-inhibiting agent, applied to the implantation site (or the implant/device surface); and/or (k) films, sponges or meshes such as INTERCEED, VICRYL mesh, and GELFOAM loaded with a fibrosis-inhibiting agent applied to the implantation site (or the implant/device surface).

A preferred polymeric matrix which can be used to help prevent the formation of fibrous or gliotic tissue around the CRM lead and electrode, either alone or in combination with a fibrosis (or gliosis) inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaeryth ritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous or gliotic tissue around the CRM lead and electrode.

It should be apparent to one of skill in the art that potentially any anti-scarring agent described herein may be utilized alone, or in combination, in the practice of this embodiment. As CRM devices, leads and electrodes are made in a variety of configurations and sizes, the exact dose administered may vary with device size, surface area and design. However, certain principles can be applied in the application of this art. Drug dose can be calculated as a function of dose per unit area (of the portion of the device being coated), total drug dose administered can be measured, and appropriate surface concentrations of active drug can be determined. Regardless of the method of application of the drug to the device (i.e., as a coating or infiltrated into the surrounding tissue), the fibrosis-inhibiting agents, used alone or in combination, may be administered under the following dosing guidelines:

Drugs and dosage: Exemplary therapeutic agents that may be used include, but are not limited to: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

Provided below are exemplary dosage ranges for various anti-scarring agents that can be used in conjunction with compositions for treating or preventing surgical adhesions in accordance with the invention. (A) Angiogenesis inhibitors including alphastatin, ZD-6474, IDN-5390, SB-2723005, ABT-518, combretastatin, and anecortane, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (B) mTOR inhibitors including AP-23573 and Temsirolimus, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (C) Tubulin antagonists including synthadotin, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (D) Epithilones including ixabepilone and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (E) Kinesin Antagonists including SB-715992 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (F) TNF alpha antagonists including etanercept, humicade, adalimumab and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (G) AKT inhibitor including erucylphosphocholine and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (H) FGF Inhibitors including IDN-5390 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (I) Collagenase Antagonists including S-0885 and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface. (J) NF KAPPA B Inhibitors including bortezomib and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (K) Elongation Factor-1 alpha inhibitors including aplidine and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface. (L) Tyrosine kinase inhibitors including gefitinib and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface.

Additional examples of anti-scarring agents which can be used include those having a high potency in the assays described herein (approximately 1-100 nM IC₅₀ range) such as isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, and tacrolimus. For high potency drugs, the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁻⁴ M of agent should be maintained on the implant or barrier surface.

Other examples of agents which can be used include those having a mid-potency in the assays described herein (approximately 100-500 nM IC₅₀ range) such as loteprednol etabonate, juglone, prednisolone, puromycin, 3-BAABE, cladribine, and mannose-6-phosphate. For mid-potency drugs, the total dose typically should not to exceed 500 mg (range of 1.0 μg to 500 mg) and preferably 1 μg to 200 mg; dose per unit area of 0.01 μg-200 μg per mm², preferably 0.1 μg/mm²-40 μg/mm²; and minimum concentration of 10⁻⁸-10⁻⁴ M of agent should be maintained on the implant or barrier surface.

Other examples of agents which can be used include those having a low potency in the assays described herein (approximately 500-1000 nm range IC₅₀ range) such as 5-azacytidine, Ly333531 (ruboxistaurin), and simvastatin. For low potency drugs, the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm²; and minimum concentration of 10⁻⁸-10⁻⁴ M of agent should to be maintained on the implant or barrier surface.

B. Therapeutic Agents for Use with Electrical Medical Devices and Implants

As described previously, numerous therapeutic agents are potentially suitable to inhibit fibrous (or glial) tissue accumulation around the device bodies, leads and electrodes of implantable electrical devices, e.g., neurostimulation and cardiac rhythm management devices. The invention provides for devices that include an agent that inhibits this tissue accumulation in the vicinity of the device, i.e., between the medical device and the host into which the medical device is implanted. The agent is therefore effective for this goal, is present in an amount that is effective to achieve this goal, and is present at one or more locations that allow for this goal to be achieved, and the device is designed to allow the beneficial effects of the agent to occur. Also, these therapeutic agents can be used alone, or in combination, to prevent scar (or glial) tissue build-up in the vicinity of the electrode-tissue interface in order to improve the clinical performance and longevity of these implants.

Agents which may inhibit fibrosis or gliosis may be readily identified based upon in vitro and in vivo (animal) models, such as those provided in Examples 39-52. Depending on their mechanisms, various agents may be effective in some, but not all screening assays for anti-fibrosis or anti-gliosis agents, and different agents may be effective in different screening assays. Agents which inhibit fibrosis can be identified through in vivo models including inhibition of intimal hyperplasia development in the rat balloon carotid artery model (Examples 44 and 61). The assays set forth in Examples 43 and 51 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. In certain embodiments, the agent may have an IC₅₀ for inhibition of cell proliferation of less than about 10,000 nM; or less than about 1000 nM; or less than about 100 nM. The assay set forth in Example 47 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. In one aspect of the invention, the agent has an IC₅₀ for inhibition of cell migration within a range of about 10⁻⁶ to about 10⁻⁹M. In certain embodiments, the agent may have an IC₅₀ for inhibition of fibroblast or smooth muscle cell migration of less than about 10,000 nM; or less than about 1000 nM; or less than about 100 nM. Assays set forth herein may be used to determine whether an agent is able to inhibit inflammatory processes, including nitric oxide production in macrophages (Example 39), and/or TNF-alpha production by macrophages (Example 40), and/or IL-1 beta production by macrophages (Example 48), and/or IL-8 production by macrophages (Example 49), and/or inhibition of MCP-1 by macrophages (Example 50). In one aspect of the invention, the agent has an IC₅₀ for inhibition of any one of these inflammatory processes within a range of about 10⁻⁶ to about 10⁻¹⁰M. In certain embodiments, the agent may have an IC₅₀ for any one of these inflammatory processes of less than about 10,000 nM; or less than about 1000 nM; or less than about 100 nM. The assay set forth in Example 45 may be used to determine whether an agent is able to inhibit MMP production. In one aspect of the invention, the agent has an IC₅₀ for inhibition of MMP production within a range of about 10⁻⁴ to about 10⁻⁸M. In certain embodiments, the agent may have an IC₅₀ for inhibition of MMP production of less than about 10,000 nM; or less than about 1000 nM; or less than about 100 nM. The assay set forth in Example 46 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. In certain embodiments, the agent may have an IC₅₀ for inhibition of angiogenesis of less than about 10,000 nM; or less than about 1000 nM; or less than about 100 nM. The assay set forth in Example 52 may be used to determine whether an agent is able to inhibit MMP-1. In one aspect of the invention, the agent has an IC₅₀ for inhibition of MMP-1 within a range of about 10⁻⁶ to about 10⁻¹⁰M. In certain embodiments, the agent may have an IC₅₀ for inhibition of MMP-1 of less than about 10,000 nM; or less than about 1000 nM; or less than about 100 nM. Agents which reduce the formation of surgical adhesions may be identified through in vivo models including the rabbit surgical adhesions model (Example 42) and the rat caecal sidewall model (Example 41). These pharmacologically active agents (described below) can then be delivered at appropriate dosages (described herein) into to the tissue either alone, or via carriers (formulations are described herein), to treat the clinical problems described previously herein.

Numerous therapeutic compounds may be identified as useful in the present invention including:

1) Adensosine A2A Receptor Antagonist

In another embodiment, the fibrosis-inhibiting compound is an adensosine A2A receptor antagonist (e.g., Sch-63390 (Schering-Plough) or an A2A receptor antagonists from Almirall-Prodesfarma, SCH-58261 (CAS No. 160098-96-4), or an analogue or derivative thereof).

2) AKT Inhibitor

In another embodiment, the fibrosis-inhibiting compound is an AKT inhibitor (e.g., PKB inhibitors from DeveloGen, AKT inhibitors from Array BioPharma, Celgene, Merck & Co, Amphora, NeoGenesis Pharmaceuticals, A-443654 (Abbott Laboratories), erucylphosphocholine (AEterna Zentaris), KRX-401 (Keryx), protein kinase B inhibitors from Astex Technology, PX-316 (ProIX), or an analogue or derivative thereof).

3) Alpha 2 Integrin Antagonist

In another embodiment, the fibrosis-inhibiting compound is an alpha 2 integrin antagonist (e.g., Pharmaprojects No. 5754 (Merck KGaA), or an analogue or derivative thereof).

4) Alpha 4 Integrin Antagonist

In another embodiment, the fibrosis-inhibiting compound is an alpha 4 integrin antagonist (e.g., T-0047 (Tanabe Seiyaku), VLA-4 antagonists from Sanofi-Aventis, Merck & Co., Biogen Idec, Uriach, and Molecumetics, alpha 4 integrin antagonists from Genentech), BIO-2421 (Biogen Idec), cell adhesion inhibitors from Kaken Pharmaceuticals, CT-737 (Wyeth), CT-767 (Elan), CY-9652 (Epimmune), CY-9701 (Epimmune), fibronectin antagonists from Uriach, integrin alpha4β7 antagonists frin Wilex, Pharmaprojects No. 5972 (UCB), Pharmaprojects No. 6603 (Wyeth), TBC-3342, TBC-772, and TBC-3486 (Encysive Pharmaceuticals), TBC-4746 (Schering-Plough), or a VLA4NCAM inhibitor (Elan Pharmaceuticals), ZD-7349 (AstraZeneca), or an analogue or derivative thereof).

5) Alpha 7 Nicotinic Receptor Agonist

In another embodiment, the fibrosis-inhibiting compound is an alpha 7 nicotinic receptor agonist (e.g., AZD-0328 (AstraZeneca), galantamine (CAS No. 357-70-0) (Synaptec), MEM-3454 or nicotinic alpha-7 agonist (Memory Pharmaceuticals and Critical Therapeutics), Pharmaprojects No. 4779 (AstraZeneca), PNU-282987 (Pfizer), SSR-180711 (Sanofi-Aventis), TC-1698 or TC-5280 (Targacept), or an analogue or derivative thereof).

6) Angiogenesis Inhibitors

In one embodiment, the fibrosis-inhibiting compound is an angiogenesis inhibitor (e.g., AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (such as combretastatin A-1, A-2, A-3, A-4, A-5, A-6, B-1, B-2, B-3, B4, D-1, D-2, and combretastatin A-4 phosphate (Oxigene)), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), or an analogue or derivative thereof). In other embodiments, the angiogenesis inhibitor may be a recombinant anti-angiogenic compound such as ANGIOCOL (available from Biostratum Inc., Durham, N.C.).

7) Apoptosis Antagonists

In another embodiment, the fibrosis-inhibiting compound is an apoptosis antagonist (e.g., didemnin B, RGB-286199 (GPC Biotech), 5F-DF-203 (Cancer Research Technology), aplidine, bongkrekic acid, triammonium salt, [6]-gingerol (CAS No. 23513-14-6), or an analogue or derivative thereof).

8) Apoptosis Activators

In another embodiment, the fibrosis-inhibiting compound is an apoptosis activator (e.g., aplidine (CAS No. 137219-37-5) (PharmaMar), canfosfamide hydrochloride (CAS No. 58382-37-74 and 39943-59-6) (Telik), idronoxil (CAS No. 81267-65-4) (Novogen), OSI-461 (OSI Pharmaceuticals), DE-098 (Santen), ARQ-550RP (ArQule), ABJ-879 (Novartis), adaphostin (NIH), anticancer agents from Apogenix Biotechnology and Momenta Pharmaceuticals, anti-PARP-1 or anti-PARP-2 (Octamer), BA-1 037 (BioAxone), CP-248 (CAS No. 200803-37-8) (OSI Pharmaceuticals), EM-1421 (Erimos), IPI-504 (Infinity Pharmaceuticals), KP-372-1 (QLT), MPC-6827 (Maxim), MT-103 (Medisyn Technologies), MX-116407 or MX-126374 (Maxim), NPI-0052 (Nereus Pharmaceuticals), NVP-AEW541 (Novartis), PARP inhibitor from Agouron (Pfizer), R-306465 (Johnson & Johnson), TG-100-33 (TargeGen), a XIAP inhibitor from AEgera, ZEN-011 (AEterna Zentaris), canertinib dihydrochloride (CAS No. 289499-45-2) (Pfizer), BH31-1,3-BAABE, or an analogue or derivative thereof).

9) Beta 1 Integrin Antagonist

In another embodiment, the fibrosis-inhibiting compound is a beta 1 integrin antagonist (e.g., β-1 integrin antagonists, Berkeley Lab, or an analogue or derivative thereof).

10) Beta Tubulin Inhibitor

In another embodiment, the fibrosis-inhibiting compound is a beta tubulin inhibitor (e.g., ZEN-017 (AEterna Zentaris), laulimalide (Kosan Biosciences), or an analogue or derivative thereof).

11) Blockers of Enzyme Production in Hepatitis C

In another embodiment, the fibrosis-inhibiting compound is an agent that blocks enzyme production in hepatitis C (e.g., merimepodib (Vertex Pharmaceuticals), or an analogue or derivative thereof).

12) Bruton's Tyrosine Kinase Inhibitor

In another embodiment, the fibrosis-inhibiting compound is a Bruton's tyrosine kinase inhibitor (e.g., a Btk inhibitor from Cellular Genomics, or an analogue or derivative thereof).

13) Calcineurin Inhibitors

In another embodiment, the fibrosis-inhibiting compound is a calcineurin inhibitor (e.g., tacrolimus (LifeCycle Pharma), or an analogue or derivative thereof).

14) Caspase 3 Inhibitors

In another embodiment, the fibrosis-inhibiting compound is a caspase 3 inhibitor (e.g., NM-3 (Mercian), or an analogue or derivative thereof).

15) CC Chemokine Receptor Antagonists

In another embodiment, the fibrosis-inhibiting compound is a CC chemokine receptor antagonist (e.g., a chemokine receptor 3 antagonist, a chemokine receptor 6 antagonist, and a chemokine receptor 7 antagonist). Representative examples of CC chemokine receptor antagonists include chemokine antagonists such as the CCR7 antagonists from Neurocrine Biosciences.

In a related embodiment, the fibrosis-inhibiting compound is a CC chemokine receptor antagonist (CCR) 1, 3, & 5 (e.g., peptide T (Advanced Immuni T), a CCR3 antagonist from GlaxoSmithKline, a chemokine antagonist (Pharmaprojects No. 6322) from Neurocrine Biosciences or Merck & Co., an HIV therapy agent from ReceptoPharm (Nutra Pharma), Pharmaprojects No. 6129 (Sangamo BioSciences), or an analogue or derivative thereof).

In certain embodiments, the CCCR antagonist is a CCR2b chemokine receptor antagonist such as RS 102895 (CAS No. 300815-41-2).

16) Cell Cycle Inhibitors

In another embodiment, the fibrosis-inhibiting compound is a cell cycle inhibitor (e.g., SNS-595 (Sunesis), homoharringtonine, or an analogue or derivative thereof).

In certain embodiments, the cell cycle inhibitor is an anti-microtubule agent (e.g., synthadotin, or an analogue or derivative thereof).

In certain embodiments, cell cycle inhibitor is a microtubule stimulant (e.g., KRX-0403, or an analogue or derivative thereof).

17) Cathepsin B Inhibitor

In another embodiment, the fibrosis-inhibiting compound is a cathepsin B inhibitor (e.g., AM-4299A (Asahi Kasei Pharma), BDI-7800 (Biopharmacopae), a cathepsin B inhibitor from Axys (Celera Genomics), MDL-104903 (CAS No. 180799-56-8) (Sanofi-Aventis), NC-700 (Nippon Chemiphar), Pharmaprojects No. 2332 (Hoffmann-La Roche), Pharmaprojects No. 4884 (Takeda), Pharmaprojects No. 5134 (Nippon Chemiphar), or an analogue or derivative thereof).

18) Cathepsin K Inhibitor

In another embodiment, the fibrosis-inhibiting compound is a cathepsin K inhibitor (e.g., 462795 (GlaxoSmithKline), INPL-022-D6 (Amura Therapeutics), or an analogue or derivative thereof).

19) Cathepsin L Inhibitor

In another embodiment, the fibrosis-inhibiting compound is a cathepsin L Inhibitor (e.g., a cathepsin L inhibitor from Takeda, INPL-022-E10 (Amura Therapeutics), Pharmaprojects No. 5447 (Taiho), or an analogue or derivative thereof).

20) CD40 Antagonists

In another embodiment, the fibrosis-inhibiting compound is a CD40 antagonists (e.g., 5D12 (Chiron), ABI-793 (Novartis), an anticancer antibody from Chiron, anti-CD40 MAb-2 (Kirin Brewery), anti-CD40 (Eli Lilly), anti-CD40L antibody (UCB), a CD40 inhibitor from Apoxis, CD40 ligand inhibitor from Millennium Pharmaceuticals, a CD40/CAP inhibitor from Snow Brand, CGEN-40 (Compugen), CHIR-12.12 (Chiron), Pharmaprojects No. 5163 (Nippon Kayaku), ruplizumab (Biogen Idec), SGN-40 (Seattle Genetics), TNX-100 (Akzo Nobel), toralizumab (CAS No. 252662-47-8) (Biogen Idec), or an analogue or derivative thereof).

21) Chemokine Receptor Agonists

In another embodiment, the fibrosis-inhibiting compound is a chemokine receptor agonist (e.g., a chemokine agonist from NeuroTarget, or an analogue or derivative thereof).

22) Chymase Inhibitors

In another embodiment, the fibrosis-inhibiting compound is a chymase inhibitor (e.g., BL-3875 (Dainippon), LEX-043 (SuperGen), NK-3201 (CAS No. 204460-24-2) (Nippon Kayaku), or an analogue or derivative thereof).

23) Collagenase (Interstitial) Antagonists

In another embodiment, the fibrosis-inhibiting compound is a collagenase (interstitial) antagonist (e.g., IBFB-212543 (IBFB Pharma), Pharmaprojects No. 3762 (Sanofi-Aventis), S-0885 (CAS No. 117517-22-3) (Sanofi-Aventis), SC-40827 (CAS No. 101470-42-2) (Pfizer), or an analogue or derivative thereof).

24) CXCR (2, 4) Antagonists

In another embodiment, the fibrosis-inhibiting compound is a CXCR (2, 4) antagonist (e.g., SB-656933 (GlaxoSmithKline), AMD3100 octahydrochloride (CAS No. 155148-31-5), or an analogue or derivative thereof).

25) Cyclin Dependent Kinase Inhibitors

In another embodiment, the fibrosis-inhibiting compound is a cyclin dependent kinase (CDK) inhibitor. In certain embodiments, the cyclin dependent kinase inhibitor is a CDK-1 inhibitor. In certain embodiments, the cyclin dependent kinase inhibitor is a CDK-2 inhibitor. In certain embodiments, the cyclin dependent kinase inhibitor is a CDK-4 inhibitor. In certain embodiments, the cyclin dependent kinase inhibitor is a CDK-6 inhibitor. Representative examples of cyclin dependent kinase inhibitors include CAK1 inhibitors from GPC Biotech and Bristol-Myers Squibb, RGB-286199 (GPC Biotech), or an analogue or derivative thereof.

Additional exemplary cyclin dependent protein kinase inhibitors include an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), CGP 74514A, bohemine, olomoucine (CAS No. 101622-51-9), indole-3-carbinol (CAS No. 700-06-1), and an analogue or derivative thereof.

26) Cyclooxygenase Inhibitors

In another embodiment, the fibrosis-inhibiting compound is a cyclooxygenase inhibitor (e.g., NS-398 (CAS No. 123653-11-2), ketoprofen, or an analogue or derivative thereof). In some embodiments, the cyclooxygenase inhibitor is a COX-1 inhibitor such as triflusal, or an analogue or derivative thereof).

27) Dihydroorotate Dehydrogenase Inhibitor (DHFR) Inhibitors

In another embodiment, the fibrosis-inhibiting compound is a DHFR inhibitor (e.g., PDX (Allos Therapeutics), SC12267, sulfamerazine (CAS No. 127-79-7), or an analogue or derivative thereof).

28) Dual Integrin Inhibitor

In another embodiment, the fibrosis-inhibiting compound is a dual integrin inhibitor (e.g., R411 (Roche Pharmaceuticals), or an analogue or derivative thereof).

29) Elastase Inhibitors

In another embodiment, the fibrosis-inhibiting compound is an elastase inhibitor (e.g., orazipone, depelestat (CAS No. 506433-25-6) (Dyax), AE-3763 (Dainippon), or an analogue or derivative thereof).

30) Elongation Factor-1 Alpha Inhibitors

In another embodiment, the fibrosis-inhibiting compound is an elongation factor-1 alpha inhibitor (e.g., aplidine, or an analogue or derivative thereof).

31) Endothelial Growth Factor Antagonists

In another embodiment, the fibrosis-inhibiting compound is an endothelial growth factor (EGF) antagonist (e.g., neovastat, NM-3 (Mercian), or an analogue or derivative thereof).

32) Endothelial Growth Factor Receptor Kinase Inhibitors

In another embodiment, the fibrosis-inhibiting compound is an endothelial growth factor receptor (EGF-R) kinase inhibitor (e.g., sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), lavendustin A (CAS No. 125697-92-9), a KDR inhibitor from LG Life Sciences, CT-6685 or CT-6729 (UCB), KRN-633 or KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGF-R inhibitor such as SU 1498, a VEGFR-2 kinase inhibitor (Bristol-Myers Squibb), XL-647 (Exelixis), a KDR inhibitor from Abbott Laboratories, or an analogue or derivative thereof).

In another embodiment, the fibrosis-inhibiting compound is an endothelial growth factor receptor 2 kinase inhibitor (e.g., sorafenib tosylate, or an analogue or derivative thereof).

33) Endotoxin Antagonists

In another embodiment, the fibrosis-inhibiting compound is an endotoxin antagonist (e.g., E5564 (Eisai Pharmaceuticals), or an analogue or derivative thereof).

34) Epothilone and Tubulin Binders

In another embodiment, the fibrosis-inhibiting compound is an epothilone or tubulin binder (e.g., ixabepilone (BMS), or an analogue or derivative thereof).

35) Estrogen Receptor Antagonists

In another embodiment, the fibrosis-inhibiting compound is an estrogen receptor antagonist (e.g., ERB-041 (Wyeth), or an analogue or derivative thereof).

36) FGF Inhibitors

In another embodiment, the fibrosis-inhibiting compound is a FGF inhibitor (e.g., IDN-5390 (Indena), or an analogue or derivative thereof).

37) Farnexyl Transferase Inhibitors

In another embodiment, the fibrosis-inhibiting compound is an inhibitor of farnexyl transferase (FTI). In certain embodiments, the FTI inhibits the RAS oncogene family. Examples of FTI's include SARASAR (from Schering Corporation, Kenilworth, N.J.), or an analogue or derivative thereof.

38) Farnesvitransferase Inhibitors

In another embodiment, the fibrosis-inhibiting compound is a farnesyltransferase inhibitor (e.g., A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), or an analogue or derivative thereof).

39) FLT-3 Kinase Inhibitors

In another embodiment, the fibrosis-inhibiting compound is a FLT-3 kinase inhibitor (e.g., Amphora, or an analogue or derivative thereof).

40) FGF Receptor Kinase Inhibitors

In another embodiment, the fibrosis-inhibiting compound is a FGF receptor kinase inhibitor (e.g., MED-A300 (Gerolymatos), SSR-128129 (Sanofi-Aventis), TBC-2250 (Encysive Pharmaceuticals), XL-999 (Exelixis), or a FGF receptor kinase inhibitor from Paradigm Therapeutics, or an analogue or derivative thereof).

41) Fibrinogen Antagonists

In another embodiment, the fibrosis-inhibiting compound is a fibrinogen antagonist (e.g., AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), mevastatin, or an analogue or derivative thereof).

42) Heat Shock Protein 90 Antagonists

In another embodiment, the fibrosis-inhibiting compound is a heat shock protein 90 antagonist (e.g., SRN-005 (Sirenade), geldanamycin or a derivative thereof, such as NSC-33050 (17-allylaminogeldanamycin; 17-AAG) or 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), radicicol, Humicola fuscoatra (CAS No. 12772-57-5), or an analogue or derivative thereof).

43) Histone Deacetylase Inhibitors

In another embodiment, the fibrosis-inhibiting compound is a histone deacetylase inhibitor (e.g., FK228 (Gloucester), trichostatin A from Streptomyces sp. (CAS No. 58880-19-6), or an analogue or derivative thereof).

44) HMGCoA Reductase Inhibitors

In another embodiment, the fibrosis-inhibiting compound is an HMGCoA reductase inhibitor (e.g., an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), cerivastatin Na)CAS No. 143201-11-0), or an analogue or derivative thereof).

45) ICAM Inhibitors

In another embodiment, the fibrosis-inhibiting compound is an ICAM inhibitor (e.g., alicaforsen (CAS No. 185229-68-9) (ISIS Pharmaceuticals), an ICAM-5 modulator (such as ICAM-4 from ICOS), or an analogue or derivative thereof).

46) IL-1, ICE & IRAK Antagonists

In another embodiment, the fibrosis-inhibiting compound is an IL-1, ICE & IRAK antagonist (e.g., CJ-14877 or CP-424174 (Pfizer), NF-61 (Negma-Lerads), or an analogue or derivative thereof).

47) IL-2 Inhibitors

In another embodiment, the fibrosis-inhibiting compound is an IL-2 inhibitor (e.g., AVE 8062 (Sanofi-Aventis), or an analogue or derivative thereof).

48) Immunosuppressants

In another embodiment, the fibrosis-inhibiting compound is an immunosuppressant (e.g., teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), or an analogue or derivative thereof).

49) IMPDH (Inosine Monophosphate)

In another embodiment, the fibrosis-inhibiting compound is IMPDH (inosine monophosphate) (e.g., ribavirin (Hoffmann-La Roche) or an analogue or derivative thereof).

50) Integrin Antagonists

In another embodiment, the fibrosis-inhibiting compound is an integrin antagonist (e.g., 683699 from Glaxo Smith Kline, integrin antagonists from Jerina AG (Germany), or an analogue or derivative thereof).

51) Interleukin Antagonists

In another embodiment, the fibrosis-inhibiting compound is an interleukin antagonist (e.g., dersalazine, or an analogue or derivative thereof).

In another embodiment, the fibrosis-inhibiting compound is an interleukin 1 antagonist (e.g., NPI-1302a-3, or an analogue or derivative thereof).

52) Inhibitors of Type III Receptor Tyrosine Kinases

In another embodiment, the fibrosis-inhibiting compound is an inhibitor of type III receptor tyrosine kinase such as FLT3, PDGRF and c-KIT (e.g., MLN518 (Millenium Pharmaceuticals), or an analogue or derivative thereof).

53) Irreversible Inhibitors of Enzyme Methionine Aminopeptidase Type 2

In another embodiment, the fibrosis-inhibiting compound is an irreversible inhibitor of enzyme methionine aminopeptidase type 2 (e.g., PPI-2458 (Praecis Pharmaceuticals), or analogue or derivative thereof).

54) Isozyme-Selective Delta Protein Kinase C Inhibitors

In another embodiment, the fibrosis-inhibiting compound is an isozyme-selective delta protein kinase C inhibitor (e.g., KAI-9803 (Kai Pharmaceuticals), or an analogue or derivative thereof).

55) JAK3 Enzyme Inhibitors

In another embodiment, the fibrosis-inhibiting compound is a JAK3 enzyme inhibitor (e.g., CP-690,550 (Pfizer), or an analogue or derivative thereof).

56) JNK Inhibitors

In another embodiment, the fibrosis-inhibiting compound is a JNK inhibitor (e.g., BF-67192 (BioFocus), XG-101 or XG-102 (Xigen), or an analogue or derivative thereof).

57) Kinase Inhibitors

In another embodiment, the fibrosis-inhibiting compound is a kinase inhibitor (e.g., a kinase inhibitors from EVOTEC, or an analogue or derivative thereof).

58) Kinesin Antagonist

In another embodiment, the fibrosis-inhibiting compound is a kinesin antagonist (e.g., SB-715992 and an antifungal from Cytokinetics, or an analogue or derivative thereof).

59) Leukotriene Inhibitors and Antagonists

In another embodiment, the fibrosis-inhibiting compound is a leukotriene inhibitor or antagonist (e.g., ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 or 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (GAS No. 101910-24-1), 92532-23-5, RP 66153 (CAS No. 142422-79-5), RP 66364 (CAS No. 186912-92-5), or RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1) or SC-53228 (CAS No. 153633-01-3) (Pfizer), spaglumic acid (ZY-15106) (CAS No. 3106-85-2) or 80619-64-3 (Novartis), tipredane (CAS No. 85197-77-9) (Bristol-Myers Squibb), U-75302 (CAS No. 119477-85-9) (Pfizer), or analogue or derivative thereof).

60) MAP Kinase Inhibitors

In another embodiment, the fibrosis-inhibiting compound is a MAP kinase inhibitor (e.g., SRN-003-556 (Sirenade), AEG-3482 (AEgera), ARRY-142886 (Array BioPharma), CDP-146 (UCB), or analogue or derivative thereof).

61) Matrix Metalloproteinase Inhibitors (MMPI)

In another embodiment, the fibrosis-inhibiting compound is a matrix metalloproteinase inhibitor. A variety of MMPI's may be used in the practice of the invention. In one embodiment, the MMPI is a MMP-1 inhibitor. In another embodiment, the MMPI is a MMP-2 inhibitor. In other embodiments, the MMPI is a MMP-4, MMP-5, MMP-6, MMP-7, or MMP-8 inhibitor. Representative examples of MMPI's include glucosamine sulfate, neovastat, GM1489 (CAS No. 170905-75-6), XL784 (EXEL-01370784), TNF-a Protease Inhibitor-1 or 2 (TAPI-1 or TAPI-2), galardin, or an analogue or derivative thereof.

62) MCP-CCR2 Inhibitors

In another embodiment, the fibrosis-inhibiting compound is a MCP-CCR2 inhibitor (e.g., MLN₁₂O₂ (Millennium Pharmaceuticals), or an analogue or derivative thereof).

63) mTOR Inhibitor

In another embodiment, the fibrosis-inhibiting compound is an mTOR inhibitor (e.g., temsirolimus (CAS No. 162635-04-3) (Wyeth), or an analogue or derivative thereof).

64) mTOR Kinase Inhibitor

In another embodiment, the fibrosis-inhibiting compound is an mTOR kinase inhibitor (e.g., ABT-578 (Abbott), temsirolimus (Wyeth), AP-23573 (Ariad), or an analogue or derivative thereof).

65) Microtubule Inhibitors

In another embodiment, the fibrosis-inhibiting compound is a microtubule inhibitor (e.g., antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4 or huMy9-6-DM1 (ImmunoGen), IDN-5404 (Indena), IMO-098 or IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, dolastatin 15 (CAS No. 123884-00-4) or an analogue or derivative thereof).

In certain embodiments, the microtubule inhibitor is a microtubule polymerization inhibitor such as vincamine, or an analogue or derivative thereof).

66) MIF Inhibitors

In another embodiment, the fibrosis-inhibiting compound is a MIF inhibitor (e.g., AVP-13546 (Avanir), an MIF inhibitor from Genzyme, migration stimulation factor D, or an analogue or derivative thereof).

67) MMP (Stromolysin) Inhibitors

In another embodiment, the fibrosis-inhibiting compound is a MMP (stromolysin) inhibitor (e.g., anticancer tetracycline from Tetragenex, rhostatin (BioAxone), TIMP's from Sanofi-Aventis (CAS No. 86102-31-0), and MMP inhibitors form Cognosci and Tetragenex, or an analogue or derivative thereof).

68) Neurokinin (NK) Antagonist

In another embodiment, the fibrosis-inhibiting compound is a neurokinin (NK) antagonist (e.g., anthrotainin (CAS No. 148084-40-6) (Sanofi-Aventis), an IBS therapeutic such as SLV-332 from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600 or SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), or an analogue or derivative thereof).

69) NF Kappa B Inhibitors

In another embodiment, the fibrosis-inhibiting compound is a NF kappa B (NFKB) inhibitor (e.g., emodin (CAS No. 518-82-1), AVE-0545 or AVE-0547 (Sanofi-Aventis), bortezomib (CAS No. 179324-69-7) (Millennium Pharmaceuticals), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), Bay 11-7085, or an analogue or derivative thereof).

70) Nitric Oxide Agonists

In another embodiment, the fibrosis-inhibiting compound is a nitric oxide agonist (e.g., Acclaim, Angx-1 039 or Angx-3227 (Angiogenix), CAS-1 609 (CAS No. 158590-73-9) (Sanofi-Aventis), GCI-503 (Spear Therapeutics), HCT-3012 (CAS No. 163133-43-5) (NicOx), hydralazine+ISDN (NitroMed), isosorbide dinitrate, Diffutab (CAS No. 87-33-2) (Eurand), isosorbide mononitrate (CAS No. 16051-77-7) from AstraZeneca, Schering AGor Schwarz Pharma, LA-419 (Lacer), molsidomine (CAS No. 25717-80-0) (from Takeda and Therabel), NCX-1000, NCX-2057, or NCX-4040 (NicOx), nitric oxide (ProStrakan), nitroglycerin in the form of a nitroglycerin patch, such as DERMATRANS from (Rottapharm), nitroglycerin (CAS No. 55-63-0) (from Cellegy Pharmaceuticals, Forest Laboratories, NovaDel, Schwarz Pharma, and Watson), NO-releasing prodrugs (Inotek), OM-294DP (OM PHARMA), oxdralazine (CAS No. 27464-23-9) (Sanofi-Aventis), pirsidomine (CAS No. 132722-74-8) (Sanofi-Aventis), prostaglandin and NO donor (Cellegy Pharmaceuticals), upidosin derivatives (Recordati), or an analogue or derivative thereof).

71) Ornithine Decarboxylase Inhibitiors

In another embodiment, the fibrosis-inhibiting compound is an ornithine decarboxylase inhibitor (e.g., aplidine, or an analogue or derivative thereof).

72) p38 MAP Kinase Inhibitors

In another embodiment, the fibrosis-inhibiting compound is a p38 MAP kinase inhibitor (e.g., AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), SKF86002 (CAS No. 72873-74-6), RPR-200765A (Sanofi-Aventis), SD-282 (Johnson & Johnson), TAK-715 (Takeda), or an analogue or derivative thereof).

73) Palmitoyl-Protein Thioesterase Inhibitors

In another embodiment, the fibrosis-inhibiting compound is a palmitoyl-protein thioesterase inhibitor (e.g., aplidine, or an analogue or derivative thereof).

74) PDGF Receptor Kinase Inhibitors

In another embodiment, the fibrosis-inhibiting compound is a PDGF receptor kinase inhibitors (e.g., AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), or an analogue or derivative thereof).

75) Peroxisome Proliferator-Activated Receptor Agonists

In another embodiment, the fibrosis-inhibiting compound is a peroxisome proliferator-activated receptor (PPAR) agonists (e.g., (−)-halofenate (Metabolex), AMG-131 (Amgen), antidiabetics from Japan Tobacco, AZD-4619, AZD-8450, or AZD-8677 (AstraZeneca), DRF-1 0945 or balaglitazone (Dr Reddy's), CS-00088 or CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001 or MC-3002 (MaxoCore Pharmaceuticals), metformin HCl+pioglitazone (CAS No. 1115-70-4 and 112529-15-4) (such as ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), such as AVANDARYL or rosiglitazone maleate/metformin extend (CAS No. 155141-29-0 and 657-24-9) such as AVANDAMET, or rosiglitazone maleate+metformin, such as AVANDAMET (GlaxoSmithKline), tesaglitazar (AstraZeneca), LBM642, WY-14,643 (CAS No. 50892-23-4), or an analogue or derivative thereof).

In certain embodiments, the PPAR Agonist is a PPARα agonist such as GW7647 or fenofibric acid (CAS No. 42017-89-0), a PPAR γ agonist such as MCC-555 (CAS No. 161600-01-7), GW9662 or GW1929, a PPAERδ agonist such as GW501516, a PPARβ, and PPARδ agonist such L-165,041 (CAS No. 79558-09-1), or an analogue or derivative thereof.

76) Phosphatase Inhibitor

In another embodiment, the fibrosis-inhibiting compound is a phosphatase inhibitor (e.g., diabetes thereapy such as SQMO3, SQDM38, SQDM60 from Sequenom, Pharmaprojects No. 4191 (Sanofi-Aventis), PRL-3 inhibitors from Genzyme, WIP1 inhibitors from Amgen, or an analogue or derivative thereof).

77) Phosphodiesterase (PDE) Inhibitors

In another embodiment, the fibrosis-inhibiting compound is a phosphodiesterase (PDE) inhibitor (e.g., avanafil (Tanabe Seiyaku), dasantafil (CAS No. 569351-91-3) (Schering-Plough), A-906119 (CAS No. 134072-58-5) or DL-850 (Sanofi-Aventis), GRC-3015, GRC-3566, or GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, or IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), theobromine (CAS No. 83-67-0), papverine hydrochloride (CAS No. 61-25-6), quercetin dehydrate (CAS No. 6151-25-3), YM 976 (CAS No. 191219-80-4), irsogladine (CAS No. 57381-26-7), or an analogue or derivative thereof).

In one embodiment, the phosphodiesterase inhibitor is a phosphodiesterase III inhibitor (e.g., enoximone, or an analogue or derivative thereof). In other embodiments, the phosphodiesterase inhibitor is a phosphodiesterase IV inhibitor (e.g., fosfosal, Atopik (Barrier Therapeutics), triflusal, or an analogue or derivative thereof). In other embodiments, the phosphodiesterase inhibitor is a phosphodiesterase V inhibitor.

78) PKC Inhibitor

In another embodiment, the fibrosis-inhibiting compound is a PKC inhibitor (e.g., HMR-105509 or P-10050 (Sanofi-Aventis), JNJ-10164830 (Johnson & Johnson), Ro-31-8425 (CAS No. 131848-97-0), NPC-15437 dihydrochloride (CAS No. 136449-85-9), or an analogue or derivative thereof).

In one embodiment, the PKC inhibitor is an inhibitor of PKC beta (e.g., ruboxistaurin (Eli Lilly), or an analogue or derivative thereof).

79) Platelet Activating Factor Antagonists

In another embodiment, the fibrosis-inhibiting compound is a platelet activating factor antagonist (e.g., dersalazine, or an analogue or derivative thereof).

80) Platelet-Derived Growth Factor Receptor Kinase Inhibitors

In another embodiment, the fibrosis-inhibiting compound is a platelet-derived growth factor receptor kinase inhibitor (e.g., sorafenib tosylate, Raf or Ras inhibitors such as sorafenib tosylate from Bayer and Onyx Pharmaceuticals, or an analogue or derivative thereof).

81) Prolyl Hydroxylase Inhibitors

In another embodiment, the fibrosis-inhibiting compound is a prolyl hydroxylase inhibitor (e.g., FG-2216 (CAS No. 11096-26-7) or HIF agonists from FibroGen, or an analogue or derivative thereof).

82) Polymorphonuclear Neutrophil Inhibitors

In another embodiment, the fibrosis-inhibiting compound is a polymorphonuclear neutrophil inhibitor (e.g., orazipone, or an analogue or derivative thereof).

83) Protein Kinase B Inhibitors

In another embodiment, the fibrosis-inhibiting compound is a protein kinase B inhibitor (e.g., Akt-1 inhibitors from Amphora, or an analogue or derivative thereof).

84) Protein Kinase C Stimulants

In another embodiment, the fibrosis-inhibiting compound is a protein kinase C stimulant (e.g., bryostatin-1, or analogue or derivative thereof).

85) Purine Nucleoside Analogues

In another embodiment, the fibrosis-inhibiting compound is a purine nucleoside analogue (e.g., cladrinbine and formulations thereof, such as MYLINAX from Serone SA and IVAX Research Inc. (Miami, Fla.), or an analogue or derivative thereof).

86) Purinoreceptor P2X Antagonist

In another embodiment, the fibrosis-inhibiting compound is a purinoreceptor P2X antagonist (e.g., AZD-9056 (AstraZeneca), R-1554 (Hoffmann-La Roche), AR-C118925XX (AstraZeneca), suramin (CAS No. 129-46-4), P2Y4 receptor from Euroscreen, or an analogue or derivative thereof).

87) Raf Kinase Inhibitors

In another embodiment, the fibrosis-inhibiting compound is a Raf kinase inhibitor (e.g., sorafenib tosylate, or an analogue or derivative thereof).

88) Reversible Inhibitors of ErbB1 and ERbB2

In another embodiment, the fibrosis-inhibiting compound is a reversible inhibitor (e.g., lapatinib (GSK), or an analogue or derivative thereof).

89) Ribonucleoside Triphosphate Reductase Inhibitors

In another embodiment, the fibrosis-inhibiting compound is a cytoplasmic tyrosine kinase inhibitor such as a SRC inhibitor (e.g., SRN-004 (Sirenade), gallium maltolate (Titan Pharmaceutcals), or an analogue or derivative thereof), or an analogue or derivative thereof).

90) SDF-1 Antagonists

In another embodiment, the fibrosis-inhibiting compound is a SDF-1 antagonist (e.g., CTCE-9908 (Chemokine Therapeutics), or an analogue or derivative thereof).

91) Sheddase Inhibitor

In another embodiment, the fibrosis-inhibiting compound is a sheddase inhibitor (e.g., INCB-7839 (Incyte Corporation), or an analogue or derivative thereof).

92) SRC Inhibitors

In another embodiment, the fibrosis-inhibiting compound is a SRC inhibitor (e.g., SRN-004 (Sirenade), or an analogue or derivative thereof).

In certain embodiments, the SRC inhibitor is a SRC kinase inhibitor (e.g., AZD0530 (AstraZeneca), or an analogue or derivative thereof).

93) Stromelysin Inhibitors

In another embodiment, the fibrosis-inhibiting compound is a stromelysin inhibitor (e.g., glucosamine sulfate, or an analogue or derivative thereof).

94) Syk Kinase Inhibitors

In another embodiment, the fibrosis-inhibiting compound is a syk kinase inhibitor (e.g., R406 (Rigel), or an analogue or derivative thereof).

95) Telomerase Inhibitors

In another embodiment, the fibrosis-inhibiting compound is a telomerase inhibitor (e.g., AS-1410 (Antisoma), or an analogue or derivative thereof).

96) TGF Beta Inhibitors

In another embodiment, the fibrosis-inhibiting compound is a TGF beta inhibitor (e.g., pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists (e.g., 1090 and 1091 from Sydney; non-industrial source), TGF-βI receptor kinase inhibitors from Eli Lilly, TGF-β receptor inhibitors from Johnson & Johnson, or an analogue or derivative thereof).

97) TNFα Antagonists and TACE Inhibitors

In another embodiment, the fibrosis-inhibiting compound is a TNFα antagonist or TACE inhibitors (e.g., adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (from Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, or an analogue or derivative thereof).

98) Tumor Necrosis Factor Antagonists

In another embodiment, the fibrosis-inhibiting compound is a tumor necrosis factor (TNF) antagonist (e.g., anti-inflammatory compounds from Biota Inc., or an analogue or derivative thereof).

99) Toll Receptor Antagonists

In another embodiment, the fibrosis-inhibiting compound is a Toll receptor antagonist (e.g., E5564 (Eisai Pharmaceuticals), or an analogue or derivative thereof).

100) Tubulin Antagonist

In another embodiment, the fibrosis-inhibiting compound is a tubulin antagonist (e.g., synthadotin, KRX-0403 (Keryx Biopharmaceuticals), or an analogue or derivative thereof).

101) Tyrosine Kinase Inhibitors

In another embodiment, the fibrosis-inhibiting compound is a tyrosine kinase inhibitor (e.g., SU-011248 (e.g., SUTENT from Pfizer Inc. (New York, N.Y.), BMS-354825, PN-355 (Paracelsian Pharmaceuticals), AGN-199659 (Allergan), (e.g., AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvill MAbs from Abgenix, anti-HER2MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 13683149-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, or U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), herbimycin A, or an analogue or derivative thereof).

In certain embodiments, the tyrosine kinase inhibitor is an EGFR tyrosine kinase inhibitor such as EKB-569 (Wyeth), or an analogue or derivative thereof).

102) VEGF Inhibitors

In another embodiment, the fibrosis-inhibiting compound is a VEGF Inhibitor (e.g., AZD2171 (AstraZeneca), or an analogue or derivative thereof).

103) Vitamin D Receptor Agonists

In another embodiment, the fibrosis-inhibiting compound is a vitamin D receptor agonist (e.g., BXL-628, BXL-922 (BioXell), or an analogue or derivative thereof).

104) Histamine Receptor Antagonists

In another embodiment, the fibrosis-inhibiting compound is an histamine receptor antagonist. Certain embodiments, the histamine receptor antagonists, such as H1, H2, and H3 histamine receptor antagonists, block the production of pro-inflammatory cytokines such as TNFa and IL-1 (e.g., IL-1β). In certain embodiments, the histamine receptor antagonist inhibit NFkB activation. Representative examples of H1 histamine receptor antagonists include phenothiazines, such as promethazine, and alkylamines, such as chlorpheniramine (CAS No. 7054-11-7), brompheniramine (CAS No. 980-71-2), fexofenadine hydrochloride, promethazine hydrochloride, loratadine, ketotifen fumarate salt, and acrivastine. Other examples of histamine receptor antagonists include broad spectrum histamine receptor antagonists such as methylxanthines (e.g., theophylline, theobromine, and caffeine). Representative examples of H2 receptor antagonists include those with a histamine-like structure including cimetidine (available under the tradename TAGAMET from SmithKline Beecham Pharmaceutical Co., Wilmington, Del.), ranitidine (available under the tradename ZANTAC from Warner Lambert Company, Morris Plains, N.J.), famotidine (available under the tradename PEPCID from Merck & Co., Whitehouse Station, N.J.), nizatidine (available under the tradename AXID from Reliant Pharmaceuticals, Inc., Liberty Corner, N.J.), nizatidine, and roxatidine acetate (CAS No. 78628-28-1). Additional examples include H3 receptor antagonists (e.g., thioperamide and thioperamide maleate salt) and anti-histamines such as tricyclic dibenozoxepins, ethanolamines, ethylenediamines, piperizines, piperidines, and pthalazinones.

105) Alpha Adrenergic Receptor Antagonists

In another embodiment, the fibrosis-inhibiting compound is an alpha adrenergic receptor antagonist. Alpha adrenergic receptor antagonists may inhibit the production of pro-inflammatory cytokines such as TNFa. The alpha adrenergic receptor antagonist may be an alpha-1 and/or an alpha-2 adrenergic receptor antagonist. Representative examples of alpha-1/alpha-2 antagonists include phenoxybenzamine. In certain embodiments, the alpha adrenergic receptor antagonist is a haloalkylamine compound or a catecholamine uptake inhibitor. Representative examples of alpha-1 adrenergic receptor antagonists include phenoxybenzamine hydrochloride and prazosin, a piperizinyl quinazoline. Representative examples of alpha-2 adrenergic receptor antagonists include imadazole based compounds such as idazoxan (CAS No. 79944-56-2), idazoxan hydrochloride, and loxapine succinate salt (CAS No. 27833-64-3). Additional examples of alpha adrenergic receptor antagonists include prazosin hydrochloride.

106) Anti-Psychotic Compounds

In another embodiment, the fibrosis-inhibiting compound is an anti-psychotic compound, such as a phenothiazine compound or an analogue or derivative thereof. In some embodiments, the fibrosis-inhibiting compound is a phenothiazine derivative capable of suppressing the production of pro-inflammatory cytokines such as TNFa and/or IL-1. Representative examples of phenothiazine compounds include chlorpromazine, fluphenazine, trifluorphenazine, mesoridazine, thioridazine, and perphenazine. Other examples of anti-psychotic compounds include thioxanthines such as chlorprothixene and thiothixene, clozapine, loxapine succinate, and olanzapine.

107) CaM Kinase II Inhibitor

In another embodiment, the fibrosis-inhibiting compound is CaM kinase II inhibitor, such as a lavendustin C, or an analogue or derivative thereof.

108) CaM Kinase II Inhibitor

In another embodiment, the fibrosis-inhibiting compound is CaM kinase II inhibitor, such as a lavendustin C, or an analogue or derivative thereof.

109) G Protein Agonist

In another embodiment, the fibrosis-inhibiting compound is G protein agonist, such as aluminum fluoride, or an analogue or derivative thereof.

110) Antibiotics and Anti-Microbials

In another embodiment, the fibrosis-inhibiting compound is an antibiotic, such as apigenin (Cas No. 520-36-5), ampicillin sodium salt (CAS No. 69-52-3), puromycin, or an analogue or derivative thereof.

In another embodiment, the fibrosis-inhibiting compound is an anti-microbial agent, such as brefeldin A (CAS No. 20350-15-6), terbinafine, benzoyl peroxide, pentamidine, ornidazole, tinidazole, ketocanazole, sulconazole nitrate salt, or an analogue or derivative thereof.

111) DNA Topoisomerase Inhibitors

In another embodiment, the fibrosis-inhibiting compound is DNA topoisomerase I inhibitor, such as β-lapachone (CAS No. 4707-32-8), or an analogue or derivative thereof.

In another embodiment, the fibrosis-inhibiting compound is DNA topoisomerase II inhibitor, such as (−)-arctigenin (CAS No. 7770-78-7), aurintricarboxylic acid, or an analogue or derivative thereof.

112) Thromboxane A2 Receptor Inhibitor

In another embodiment, the fibrosis-inhibiting compound is thromboxane A2 receptor inhibitor, such as BM-531 (CAS No. 284464-46-6), ozagrel hydrochloride (CAS No. 78712-43-3), or an analogue or derivative thereof.

113) D2-Dopamine ReceptorAntagonist

In another embodiment, the fibrosis-inhibiting compound is a D2 dopamine receptor antagonist, such as clozapine (CAS No. 5786-21-0), mesoridazine benzenesulfonate, or an analogue or derivative thereof.

114) Peptidyl-Prolyl Cis/Trans Isomerase Inhibitor

In another embodiment, the fibrosis-inhibiting compound is a Peptidyl-Prolyl Cis/Trans Isomerase Inhibitor, such as juglone (CAS No. 481-39-0), or an analogue or derivative thereof.

115) Dopamine Antagonists

In another embodiment, the fibrosis-inhibiting compound is a dopamine antagonist, such as thiothixene, thioridazine hydrochloride, or an analogue or derivative thereof.

116) Anesthetics

In another embodiment, the fibrosis-inhibiting compound is an anesthetic compound, such as lidocaine (CAS No. 137-58-6), or an analogue or derivative thereof.

117) Clotting Factors

In another embodiment, the fibrosis-inhibiting compound is a clotting factor, such as menadione (CAS No. 58-27-5), or an analogue or derivative thereof.

118) Lysyl Hydrolase Inhibitor

In another embodiment, the fibrosis-inhibiting compound is a lysyl hydrolase inhibitor, such as minoxidil (CAS No. 38304-91-5), or an analogue or derivative thereof.

119) Muscarinic Receptor Inhibitor

In another embodiment, the fibrosis-inhibiting compound is a muscarinic receptor inhibitor, such as perphenazine (CAS No. 58-39-9), or an analogue or derivative thereof.

120) Superoxide Anion Generator

In another embodiment, the fibrosis-inhibiting compound is a superoxide anion generator, such as plumbagin (CAS No. 481-42-5), or an analogue or derivative thereof.

121) Steroids

In another embodiment, the fibrosis-inhibiting compound is a steroid, such as prednisolone, prednisolone 21-acetate (CAS No. 52-21-1), loteprednol etabonate, (CAS No. 82034-46-6), clobetasol propionate, or an analogue or derivative thereof.

122) Anti-Proliferative Agents

In another embodiment, the fibrosis-inhibiting compound is an anti-proliferative agent, such as silibinin (CAS No. 22888-70-6), silymarin (CAS No. 65666-07-1), 1,2-hexanediol, dioctyl phthalate (CAS No. 117-81-7), zirconium (IV) oxide, glycyrrhizic acid, spermidine trihydrochloride or tetrahydrochloride, CGP 74514A, spermine tetrahydrochloride, NG-methyl-L-arginine acetate salt, galardin, halofuginone hydrobromide (HBr), fascaplysin, or an analogue or derivative thereof.

123) Diuretics

In another embodiment, the fibrosis-inhibiting compound is a diuretic, such as spironolactone (CAS No. 52-01-7), or an analogue or derivative thereof.

124) Anti-Coagulants

In another embodiment, the fibrosis-inhibiting compound is an anti-coagulant, such as fucoidan from Fucus vesiculosus (CAS No. 9072-19-9), or an analogue or derivative thereof.

125) Cyclic GMP Agonists

In another embodiment, the fibrosis-inhibiting compound is a cyclic GMP agonist, such as sinitrodil (CAS No. 143248-63-9), or an analogue or derivative thereof.

126) Adenylate Cyclase Agonist

In another embodiment, the fibrosis-inhibiting compound is an adenylate cyclase agonist, such as histamine (CAS No. 51-45-6), or an analogue or derivative thereof.

127) Antioxidants

In another embodiment, the fibrosis-inhibiting compound is an antioxidant, such as morpholine, phytic acid dipotassium salt, (−)-epigallocatechin or (−)-epigallocatechin gallate from green tea (CAS Nos. 970-74-1 and 1257-08-5, respectively), (−)-epigallocatechin gallate (CAS No. 989-51-5), nobiletin (CAS No. 478-01-3), probucol (CAS No. 23288-49-5), phosphorous acid, hesperetin, L-ascorbyl-2-phosphate, magnesium salt (CAS No. 84309-23-9), catechin, (±)-naringenin (CAS No. 67604-48-2), (−)-epicatechin, (−)-epicatechin gallate, 3-hydroxyflavone, (−)-arctigenin, or an analogue or derivative thereof.

128) Nitric Oxide Synthase Inhibitors

In another embodiment, the fibrosis-inhibiting compound is a nitric oxide synthase inhibitor, such as ammonium pyrrolidinedithiocarbamate (CAS No. 5108-96-3), or an analogue or derivative thereof.

In another embodiment, the fibrosis-inhibiting compound is a reversible nitric oxide synthase inhibitor, such as NB-methyl-L-arginine acetate salt (L-NMMA) (CAS No. 53308-83-1), or an analogue or derivative thereof.

129) Anti-Neoplastic Agents

In another embodiment, the fibrosis-inhibiting compound is an anti-neoplastic agent, such as tirapazamine (CAS No. 27314-97-2), fludarabine (CAS No. 21679-14-1), cladribine, imatinib mesilate, or an analogue or derivative thereof.

130) DNA Synthesis Inhibitors

In another embodiment, the fibrosis-inhibiting compound is a DNA synthesis inhibitor, such as S-(2-hydroxy-5-nitrobenzyl)-6-thioguanosine or uracilfludarabine phosphate (CAS No. 75607-67-9), 6,11-dihydroxy-5,12-naphthacenedione, or an analogue or derivative thereof.

131) DNA Alkylating Agents

In another embodiment, the fibrosis-inhibiting compound is a DNA alkylating agent, such as dacarbazine (CAS No. 4342-03-4), temozolomide, procarbazine HCl, or an analogue or derivative thereof.

132) DNA Methylation Inhibitors

In another embodiment, the fibrosis-inhibiting compound is a DNA methylation inhibitor, such as decitabine (CAS No. 2353-33-5), or an analogue or derivative thereof.

133) NSAID Agents

In another embodiment, the fibrosis-inhibiting compound is a NSAID agent, such as nabumetone, benzydamine hydrochloride, or an analogue or derivative thereof.

134) Pertidvlglycine Alpha-Hydroxylating Monooxygenase Inhibitors

In another embodiment, the fibrosis-inhibiting compound is a peptidylglycine alpha-hydroxylating monooxygenase inhibitor, such as trans-styrylacetic acid, or an analogue or derivative thereof.

135) MEK1/MEK2 Inhibitors

In another embodiment, the fibrosis-inhibiting compound is a MEK1/MEK 2 inhibitor, such as U0126 (CAS No. 109511-58-2), or an analogue or derivative thereof.

136) NO Synthase Inhibitors

In another embodiment, the fibrosis-inhibiting compound is an NO synthase inhibitor, such as L-NAME (CAS No. 53308-83-1), NG-Methyl-L-arginine acetate salt, or an analogue or derivative thereof.

137) Retinoic Acid Receptor Antagonists

In another embodiment, the fibrosis-inhibiting compound is retinoic acid receptor antagonist, such as isotretinoin (CAS No. 4759-48-2), or an analogue or derivative thereof.

138) ACE Inhibitors

In another embodiment, the fibrosis-inhibiting compound is an ACE inhibitor, such as quinapril hydrochloride (CAS No. 85441-61-8), enalapril, or an analogue or derivative thereof.

139) Glycosylation Inhibitors

In another embodiment, the fibrosis-inhibiting compound is a glycosylation inhibitor, such as aminoguanidine hydrochloride, castanospermine, or an analogue or derivative thereof.

140) Intracellular Calcium Influx Inhibitors

In another embodiment, the fibrosis-inhibiting compound is an intracellular calcium influx inhibitor, such as TAS-301 (CAS No. 193620-69-8), or an analogue or derivative thereof.

141) Anti-Emetic Agents

In another embodiment, the fibrosis-inhibiting compound is an anti-emetic agent, such as amifostine (CAS No. 20537-88-6), or an analogue or derivative thereof.

142) Acetylcholinesterase Inhibitors

In another embodiment, the fibrosis-inhibiting compound is an acetylcholinesterase inhibitor, such as (−)-huperzine A (CAS No. 102518-79-6), or an analogue or derivative thereof.

143) ALK-5 Receptor Antagonists

In another embodiment, the fibrosis-inhibiting compound is an ALK-5 receptor antagonist, such as SB 431542 (CAS No. 301836-41-9), or an analogue or derivative thereof.

144) RAR/RXR Antagonists

In another embodiment, the fibrosis-inhibiting compound is a RAR/RXT antagonist, such as 9-cis-retinoic acid, or an analogue or derivative thereof.

145) EIF-2a Inhibitors

In another embodiment, the fibrosis-inhibiting compound is a eIF-2a inhibitor, such as salubrinal, or an analogue or derivative thereof.

146) S-Adenosyl-L-Homocysteine Hydrolase Inhibitors

In another embodiment, the fibrosis-inhibiting compound is a S-adenosyl-L-homocysteine hydrolase inhibitor, such as 3-deazaadenosine, or an analogue or derivative thereof.

147) Estrogen Agonists

In another embodiment, the fibrosis-inhibiting compound is an estrogen agonist, such as coumestrol, bisphenol A, 1-linoleoyl-rac-glycerol (CAS No. 2277-28-3), daidzein (4,7-dihydroxy-iso-flavone), dihexyl phthalate, kaempferol, formononetin, or an analogue or derivative thereof.

148) Serotonin Receptor Inhibitors

In another embodiment, the fibrosis-inhibiting compound is a serotonin receptor inhibitor, such as amitriptyline hydrochloride, or an analogue or derivative thereof.

149) Anti-Thrombotic Agents

In another embodiment, the fibrosis-inhibiting compound is an anti-thrombotic agent, such as geniposidic acid, geniposide, or an analogue or derivative thereof.

150) Tryptase Inhibitors

In another embodiment, the fibrosis-inhibiting compound is a tryptase inhibitors, such as 2-azetidinone, or an analogue or derivative thereof.

151) Pesticides

In another embodiment, the fibrosis-inhibiting compound is a pesticide, such as allyl disulfide, or an analogue or derivative thereof.

152) Bone Mineralization Promotor

In another embodiment, the fibrosis-inhibiting compound is a bone mineralization promotor, such as glycerol 2-phosphate disodium salt hydrate, or an analogue or derivative thereof.

153) Bisphosphonate Compounds

In another embodiment, the fibrosis-inhibiting compound is a bisphosphonate compound, such as risedronate, or an analogue or derivative thereof.

154) Anti-Inflammatory Compounds

In another embodiment, the fibrosis-inhibiting compound is an anti-inflammatory compound, such as aucubin, cepharanthine, or an analogue or derivative thereof.

155) DNA Methylation Promotors

In another embodiment, the fibrosis-inhibiting compound is a DNA methylation promotor, such as 5-azacytidine, or an analogue or derivative thereof.

156) Anti-Spasmodic Agents

In another embodiment, the fibrosis-inhibiting compound is an anti-spasmodic agent, such as 2-hydroxy-4,6-dimethoxyacetophenone, or an analogue or derivative thereof.

157) Protein Synthesis Inhibitors

In another embodiment, the fibrosis-inhibiting compound is a protein synthesis inhibitor, such as oxytetracycline hydrochloride, or an analogue or derivative thereof.

158) α-Glucosidase Inhibitors

In another embodiment, the fibrosis-inhibiting compound is a α-glucosidase inhibitor, such as myricetin (CAS No. 529-44-2), or an analogue or derivative thereof.

159) Calcium Channel Blockers

In another embodiment, the fibrosis-inhibiting compound is a calcium channel blocker, such as verapamil, nitrendipine, or an analogue or derivative thereof.

In another embodiment, the fibrosis-inhibiting compound is a L-type calcium channel blocker, such as nifedipine (CAS No. 21829-25-4), (+)-cis-diltiazem hydrochloride, or an analogue or derivative thereof.

In another embodiment, the fibrosis-inhibiting compound is a T-type calcium channel blocker, such as penfluridol (CAS No. 26864-56-2), or an analogue or derivative thereof.

160) Pyruvate Dehydrogenase Activators

In another embodiment, the fibrosis-inhibiting compound is a pyruvate dehydrogenase activator, such as dichloroacetic acid, or an analogue or derivative thereof.

161) Prostaglandin Inhibitors

In another embodiment, the fibrosis-inhibiting compound is a prostaglandin inhibitor, such as betulinic acid, or an analogue or derivative thereof.

162) Sodium Channel Inhibitors

In another embodiment, the fibrosis-inhibiting compound is a sodium channel inhibitor, such as amiloride hydrochloride hydrate, or an analogue or derivative thereof.

163) Serine Protease Inhibitors

In another embodiment, the fibrosis-inhibiting compound is a serine protease inhibitor, such as gabexate mesylate, or an analogue or derivative thereof.

164) Intracellular Calcium Flux Inhibitors

In another embodiment, the fibrosis-inhibiting compound is an intracellular calcium flux inhibitor, such as thapsigargin, or an analogue or derivative thereof.

165) JAK2 Inhibitors

In another embodiment, the fibrosis-inhibiting compound is a JAK2 inhibitor (e.g., AG-490 (CAS No. 134036-52-5), or an analogue or derivative thereof).

166) Androgen Inhibitors

In another embodiment, the fibrosis-inhibiting compound is an androgen inhibitor (e.g., tibolone (CAS No. 5630-53-5), or an analogue or derivative thereof).

167) Aromatase Inhibitors

In another embodiment, the fibrosis-inhibiting compound is an aromatase inhibitor (e.g., letrozole, or an analogue or derivative thereof).

168) Anti-Viral Agents

In another embodiment, the fibrosis-inhibiting compound is an anti-viral agent, such as imiquimod, or an analogue or derivative thereof.

169) 5-HT Inhibitors

In another embodiment, the fibrosis-inhibiting compound is a 5-HT inhibitor, such as ketanserin tartrate, amoxapine, or an analogue or derivative thereof.

170) FXR Antagonists

In another embodiment, the fibrosis-inhibiting compound is a FXR antagonist, such as guggulsterone (CAS No. 95975-55-6), or an analogue or derivative thereof.

171) Actin Polymerization and Stabilization Promotors

In another embodiment, the fibrosis-inhibiting compound is an actin polymerization and stabilization promotor, such as jasplakinolide, or an analogue or derivative thereof.

172) AXOR12 Agonists

In another embodiment, the fibrosis-inhibiting compound is an AXOR12 agonist, such as metastin (KiSS-1 (112-121), or an analogue or derivative thereof.

173) Angiotensin II Receptor Antagonists

In another embodiment, the fibrosis-inhibiting compound is an angiotensin II receptor agonist, such as losartan potassium, or an analogue or derivative thereof.

174) Platelet Aggregation Inhibitors

In another embodiment, the fibrosis-inhibiting compound is a platelet aggregation inhibitor, such as clopidogrel, or an analogue or derivative thereof.

175) CB1/CB2 Receptor Agonists

In another embodiment, the fibrosis-inhibiting compound is a CB1/CB2 receptor agonist, such as HU-210 (CAS No. 112830-95-2), or an analogue or derivative thereof.

176) Norepinephrine Reuptake Inhibitors

In another embodiment, the fibrosis-inhibiting compound is a norepinephrine reuptake inhibitor, such as nortriptyline hydrochloride, or an analogue or derivative thereof.

177) Selective Serotonin Reuptake Inhibitors

In another embodiment, the fibrosis-inhibiting compound is a selective serotonin reuptake inhibitor, such as paroxetine maleate, or an analogue or derivative thereof.

178) Reducing Agents

In another embodiment, the fibrosis-inhibiting compound is a reducing agent such as WW-85 (Inotek), or an analogue or derivative thereof.

179) Immuno-Modulators

In another embodiment, the fibrosis-inhibiting compound is an immunomodulators such as Bay 11-7085, (−)-arctigenin, idazoxan hydrochloride, or an analogue or derivative thereof.

### C. Combination Therapies

In addition to incorporation of a fibrosis-inhibiting agent, one or more other pharmaceutically active agents can be incorporated into the present compositions to improve or enhance efficacy. In one aspect, the composition may further include a compound which acts to have an inhibitory effect on pathological processes in or around the treatment site. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors, tyrosine kinase inhibitors, MMP inhibitors, p38 MAP kinase inhibitors, immunosuppressants, apoptosis antagonists, caspase inhibitors, and JNK inhibitors.

In one aspect, the present invention also provides for the combination of an electrical device (as well as compositions and methods for making electrical devices) that includes an anti-fibrosing agent and an anti-infective agent, which reduces the likelihood of infections.

Infection is a common complication of the implantation of foreign bodies such as, for example, medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based the assays provided in Example 56. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

a) Anthracyclines

Anthracyclines have the following general structure, where the R groups may be a variety of organic groups:

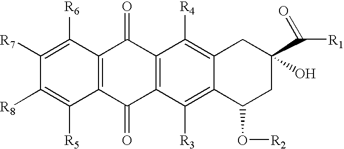

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

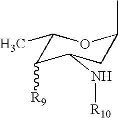

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

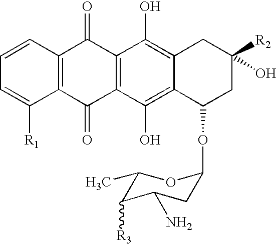

| | R₁ | R₂ | R₃ |
|---|---|---|---|
| Doxorubicin: | OCH₃ | C(O)CH₂OH | OH out of ring plane |
| Epirubicin: (4′ epimer | OCH₃ | C(O)CH₂OH | OH in ring plane |
| of doxorubicin) | | | |
| Daunorubicin: | OCH₃ | C(O)CH₃ | OH out of ring plane |
| Idarubicin: | H | C(O)CH₃ | OH out of ring plane |
| Pirarubicin: | OCH₃ | C(O)CH₂OH | 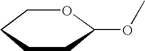 |
| Zorubicin: | OCH₃ | C(CH₃)(═N)NHC(O)C₆H₅ | OH |
| Carubicin: | OH | C(O)CH₃ | OH out of ring plane |

Other suitable anthracyclines are anthramycin, mitoxantrone, menogaril, nogalamycin, aclacinomycin A, olivomycin A, chromomycin A₃, and plicamycin having the structures:

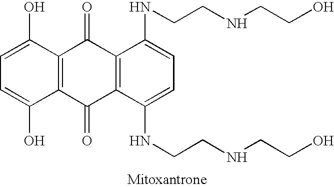

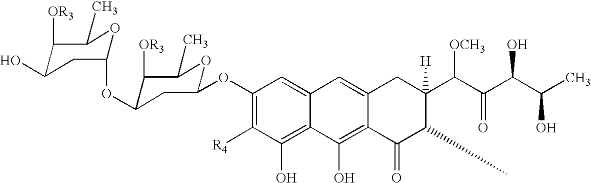

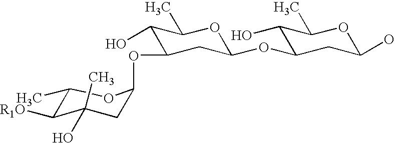

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |
| | R₁ | R₂ | R₃ | |
| Menogaril | H | OCH₃ | H | |
| Nogalamycin | O-sugar | H | COOCH₃ | |

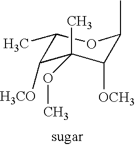

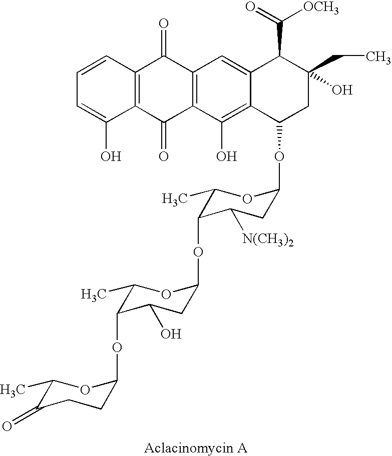

Other representative anthracyclines include, FCE 23762, a doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Natl Cancer Inst. 89(16): 1217-1223, 1997), 4-demethoxy-7-O-[2,6-d]deoxy-4-O-(2,3,6-trideoxy-3-amino-α-L-lyxo-hexopyranosyl)-α-L-lyxo-hexopyranosyl]-adriamicinone doxorubicin disaccharide analogue (Monteagudo et al., Carbohydr. Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Wiliner et al., Bioconjugate Chem. 4(6):521-7,1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14): 3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ, 16(Biol. 1):21-7, 1988), 4′-deoxydoxorubicin (Schoeizel et al., Leuk. Res. 10(12):1455-9, 1986), 4-demethyoxy-4′-o-methyldoxorubicin (Giuliani et al., Proc Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl)doxorubicin derivatives (U.S. Pat. No. 4,314,054), 3′-deamino-3′-(4-mortholinyl)doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan &Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydroxorubicin; (3′-deamino-3′-(3″-cyano-4″-morpholinyl)daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl)doxorubicin derivatives (U.S. Pat. No. 4,314,054) and 3-deamino-3-(4-morpholinyl)doxorubicin derivatives (U.S. Pat. No. 4,301,277).

b) Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

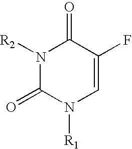

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

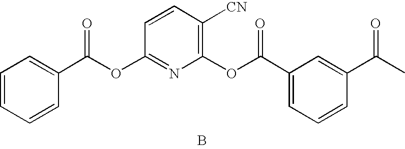

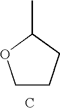

Other suitable fluoropyrimidine analogues include 5-FudR (5-fluoro-deoxyuridine), or an analogue or derivative thereof, including 5-iododeoxyuridine (5-ludR),5-bromodeoxyuridine (5-BudR), fluorouridine triphosphate (5-FUTP), and fluorodeoxyuridine monophosphate (5-dFUMP). Exemplary compounds have the structures:

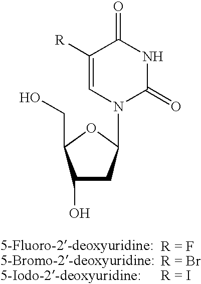

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al, Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3): 144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluoyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

c) Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

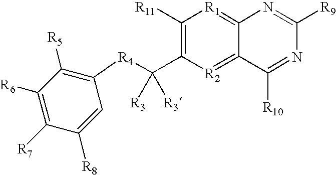

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

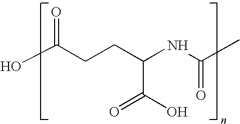

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

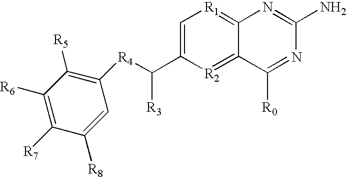

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A (n = 1) | H |
| Peritrexim | NH₂ | N | C(CH₃) | H | single bond | OCH₃ | H | H | OCH₃ |

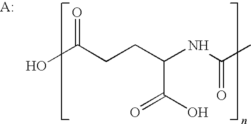

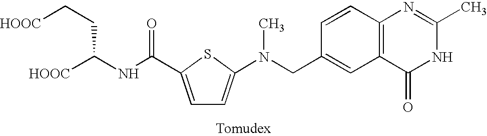

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al, Biol. Pharm. Bull. 18(11): 1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified ornithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm. Biopharm. Pharm. Technol., 5634, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl)methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol 42(12):2400-3, 1991), β,γ-methano methotrexate analogues (Rosowsky et al., Pteridines 2(3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int Symp. Pteridines Folic Acid Deriv., 1154-7, 1989), N-(L-α-aminoacyl)methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (4,725,687), Nδ-acyl-Nα-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2, .omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122 (Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7): 1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1 308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220);

These compounds are believed to act as antimetabolites of folic acid.

d) Podophyllotoxins

In another aspect, the therapeutic agent is a podophyllotoxin, or a derivative or an analogue thereof. Exemplary compounds of this type are etoposide or teniposide, which have the following structures:

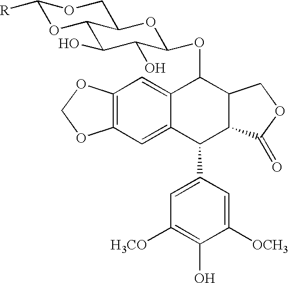

| | R |
|---|---|
| Etoposide | CH₃ |
| Teniposide | 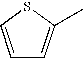 |

Other representative examples of podophyllotoxins include Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

e) Camptothecins

In another aspect, the therapeutic agent is camptothecin, or an analogue or derivative thereof. Camptothecins have the following general structure.

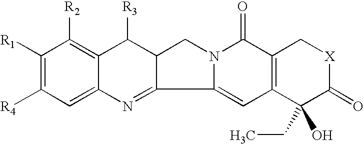

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

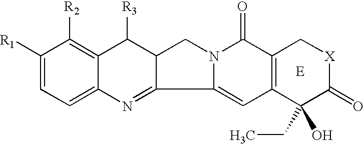

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

f) Hydroxyureas

The therapeutic agent of the present invention may be a hydroxyurea. Hydroxyureas have the following general structure:

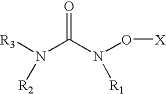

Suitable hydroxyureas are disclosed in, for example, U.S. Pat. No. 6,080,874, wherein R₁ is:

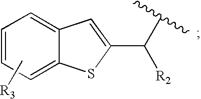

and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form:

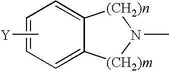

wherein m is 1 or 2, n is 0-2 and Y is an alkyl group.

In one aspect, the hydroxyurea has the structure:

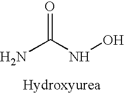

These compounds are thought to function by inhibiting DNA synthesis.

g) Platinum Complexes

In another aspect, the therapeutic agent is a platinum compound. In general, suitable platinum complexes may be of Pt(II) or Pt(IV) and have this basic structure:

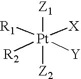

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

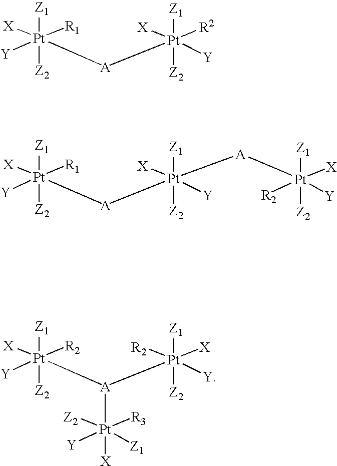

Exemplary platinum compounds are cisplatin, carboplatin, oxaliplatin, and miboplatin having the structures:

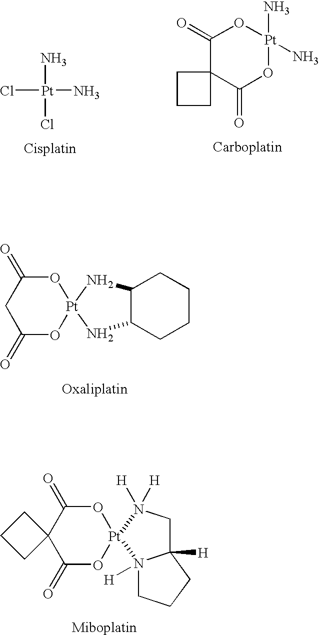

Other representative platinum compounds include (CPA)₂Pt[DOLYM] and (DACH)Pt[DOLYM] cisplatin (Choi et al., Arch. Pharmacal Res. 22(2): 151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg Chem. 36(25):5969-5971, 1997), 4-pyridoxate diammine hydroxy platinum (Tokunaga et al., Pharm. Sci. 3(7):353-356, 1997), Pt(II) . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(OAc)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg Biochem. 62(1):75, 1996), cis-1,4-diaminocyclohexane cisplatin analogues (Shamsuddin et al, J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int J. Oncol 5(3):597-602, 1994), cis-diaminedichloroplatinum(II) and its analogues cis-1,1-cyclobutanedicarbosylato(2R)-2-methyl-1,4-butaned iam ineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(I) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethyle ned iamine) dichloroplatinum(II) (Bednarski et al., J. Med. Chem. 35(23):4479-85, 1992), cisplatin analogues containing a tethered dansyl group (Hartwig et al., J. Am. Chem. Soc 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexanemalonatoplatinum (II) (Oswald et al, Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al, Eur. J. Cancer Clin. Oncol 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl 10(1); 139-45, 1987), (NPr4)2((PtCL4).cis-(PtCl2-(NH₂Me)₂)) (Brammer et al., J. Chem. Soc, Chem. Commun. 6:443-5, 1987), aliphatic tricarboxylic acid platinum complexes (EPA 185225), and cis-dichloro(amino acid) (tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

As medical implants are made in a variety of configurations and sizes, the exact dose administered may vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the cardiac implant, the preferred anticancer agents, used alone or in combination, may be administered under the following dosing guidelines:

(a) Anthracyclines. Utilizing the anthracycline doxorubicin as an example, whether applied as a polymer coating, incorporated into the polymers which make up the implant components, or applied without a carrier polymer, the total dose of doxorubicin applied to the implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings may release doxorubicin at differing rates, the above dosing parameters should be utilized in combination with the release rate of the drug from the implant surface such that a minimum concentration of 10⁻⁸-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 3 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-5 μg/mm². As different polymer and non-polymer coatings will release mitoxantrone at differing rates, the above dosing parameters should be utilized in combination with the release rate of the drug from the implant surface such that a minimum concentration of 10⁻⁴-10⁻⁸ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoronyrimidines Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.05 μg-200 μg per mm² of surface area. In a particularly preferred embodiment, 5-fluorouracil should be applied to the implant surface at a dose of 0.5 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁴-10⁻⁷ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It may be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the composition.

In another aspect, an anti-infective agent (e.g., anthracyclines (e.g., doxorubicin or mitoxantrone), fluoropyrimidines (e.g., 5-fluorouracil), folic acid antagonists (e.g., methotrexate and/or podophylotoxins (e.g., etoposide)) can be combined with traditional antibiotic and/or antifungal agents to enhance efficacy. The anti-infective agent may be further combined with anti-thrombotic and/or antiplatelet agents (for example, heparin, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

In addition to incorporation of the above-mentioned therapeutic agents (i.e., anti-infective agents or fibrosis-inhibiting agents), one or more other pharmaceutically active agents can be incorporated into the present compositions and devices to improve or enhance efficacy. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

Implantable electrical devices and compositions for use with implantable electrical devices may further include an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound which prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

Compositions for use with electrical devices may be or include a hydrophilic polymer gel that itself has anti-thrombogenic properties. For example, the composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Electrical devices and compositions for use with implantable electrical devices may further include a compound which acts to have an inhibitory effect on pathological processes in or around the treatment site. In certain aspects, the agent may be selected from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2,WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be combined with implantable electrical devices according to the invention include tyrosine kinase inhibitors, such as imantinib, ZK-222584, CGP-52411, CGP-53716, NVP-AAK980-NX, CP-127374, CP-564959, PD-171026, PD-173956, PD-1 80970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and dexlipotam; p38 MAP kinase inhibitors such as include CGH-2466 and PD-98-59; immunosuppressants such as argyrin B, macrocyclic lactone, ADZ-62-826, CCI-779, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo(b,f)oxepin); and caspase inhibitors (e.g., PF-5901 (benzenemethanol, alpha-pentyl-3-(2-quinolinylmethoxy)-), and JNK inhibitor (e.g., AS-602801).

In another aspect, the electrical device may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

In certain aspects, a polymeric composition comprising a fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin,) and a CYP inhibitor, which may be combined (e.g., coated) with any of the devices described herein. Representative examples of CYP inhibitors include flavones, azole antifungals, macrolide antibiotics, HIV protease inhibitors, and anti-sense oligomers. Devices comprising a combination of a fibrosis-inhibiting agent and a CYP inhibitor may be used to treat a variety of proliferative conditions that can lead to undesired scarring of tissue, including intimal hyperplasia, surgical adhesions, and tumor growth.

Within various embodiments of the invention, a device incorporates or is coated on one aspect, portion or surface with a composition which inhibits fibrosis (and/or restenosis), as well as with a composition or compound which promotes fibrosis on another aspect, portion or surface of the device. Representative examples of agents that promote fibrosis include silk and other irritants (e.g., talc, wool (including animal wool, wood wool, and synthetic wool), talcum powder, copper, metallic beryllium (or its oxides), quartz dust, silica, crystalline silicates), polymers (e.g., polylysine, polyurethanes, poly(ethylene terephthalate), PTFE, poly(alkylcyanoacrylates), and poly(ethylene-co-vinylacetate); vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-β (TGF-β-1, TGF-β-2, TGF-β-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor—PIGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNFα), nerve growth factor (NGF), interferon-α, interferon-β, histamine, endothelin-1, angiotensin II, growth hormone (GH), and synthetic peptides, analogues or derivatives of these factors are also suitable for release from specific implants and devices to be described later. Other examples include CTGF (connective tissue growth factor); inflammatory microcrystals (e.g., crystalline minerals such as crystalline silicates); bromocriptine, methylsergide, methotrexate, chitosan, N-carboxybutyl chitosan, carbon tetrachloride, thioacetamide, fibrosin, ethanol, bleomycin, naturally occurring or synthetic peptides containing the Arg-Gly-Asp (RGD) sequence, generally at one or both termini (see, e.g., U.S. Pat. No. 5,997,895), and tissue adhesives, such as cyanoacrylate and crosslinked poly(ethylene glycol)-methylated collagen compositions. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

Other representative examples of fibrosis-inducing agents include components of extracellular matrix (e.g., fibronectin, fibrin, fibrinogen, collagen (e.g., bovine collagen), including fibrillar and non-fibrillar collagen, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as TIMPs (tissue inhibitors of matrix metalloproteinases) and synthetic TIMPs, such as, e.g., marimistat, batimistat, doxycycline, tetracycline, minocycline, TROCADE, Ro-1130830, CGS 27023A, and BMS-275291 and analogues and derivatives thereof.

Although the above therapeutic agents have been provided for the purposes of illustration, it may be understood that the present invention is not so limited. For example, although agents are specifically referred to above, the present invention may be understood to include analogues, derivatives and conjugates of such agents. As an illustration, combretastatin A4 may be understood to refer to not only the common chemically available form of combretastatin, but analogues (e.g., combretastatin A2, A3, A5, A6, as noted above) and combretastatin conjugates. In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

Dosages

Since neurostimulation devices and cardiac rhythm management devices are made in a variety of configurations and sizes, the exact dose administered may vary with device size, surface area and design. However, certain principles can be applied in the application of this art. Drug dose can be calculated as a function of dose (i.e., amount) per unit area of the portion of the device being coated. Surface area can be measured or determined by methods known to one of ordinary skill in the art. Total drug dose administered can be measured and appropriate surface concentrations of active drug can be determined. Drugs are to be used at concentrations that range from several times more than to 10%, 5%, or even less than 1% of the concentration typically used in a single chemotherapeutic systemic dose application. In certain aspects, the drug is released in effective concentrations for a period ranging from 1-90 days. Regardless of the method of application of the drug to the device, the fibrosis-inhibiting agents, used alone or in combination, should be administered under the following dosing guidelines:

As described above, electrical devices may be used in combination with a composition that includes an anti-scarring agent. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

For high potency drugs (approximately 1-100 nM) IC₅₀ range in assays described herein), the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁻⁴ M of agent should be maintained on the implant or barrier surface. For mid-potency agents (approximately 100-500 nM IC₅₀ range), the total dose typically should not to exceed 500 mg (range of 1.0 μg to 500 mg) and preferably 1 μg to 200 mg; dose per unit area of 0.01 μg-200 μg per mm², preferably 0.1 μg/mm²-40 μg/mm². For low potency drugs (approximately 500-1000 nM IC₅₀ range), the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm².

It should be apparent to one of skill in the art that potentially any anti-scarring agent described above may be utilized alone, or in combination, in the practice of this embodiment.

In various aspects, the present invention provides a medical device that contains an anti-fibrosing agent listed below in a dosage as set forth above: 1) an anti-fibrotic agent that inhibits cell regeneration, 2) an anti-fibrotic agent that inhibits angiogenesis, 3) an anti-fibrotic agent that inhibits fibroblast migration, 4) an anti-fibrotic agent that inhibits fibroblast proliferation, 5) an anti-fibrotic agent that inhibits deposition of extracellular matrix, 6) an anti-fibrotic agent inhibits tissue remodeling, 7) an adensosine A2A receptor antagonist, 8) an AKT inhibitor, 9) an alpha 2 integrin antagonist, wherein the alpha 2 integrin antagonist is Pharmaprojects No. 5754 (Merck KgaA), 10) an alpha 4 integrin antagonist, 11) an alpha 7 nicotinic receptor agonist, 12) an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-1111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof, 13) an apoptosis antagonist, 14) an apoptosis activator, 15) a beta 1 integrin antagonist, 16) a beta tubulin inhibitor, 17) a blocker of enzyme production in Hepatitis C, 18) a Bruton's tyrosine kinase inhibitor, 19) a calcineurin inhibitor, 20) a caspase 3 inhibitor, 21) a CC chemokine receptor antagonist, 22) a cell cycle inhibitor selected from the group consisting of SNS-595 (Sunesis), synthadotin, KRX-0403, homoharringtonine, and an analogue or derivative thereof, 23) a cathepsin B inhibitor, 24) a cathepsin K inhibitor, wherein the cathepsin K inhibitor is 462795 (GlaxoSmithKline), INPL-022-D6 (Amura Therapeutics), or an analogue or derivative thereof, 25) a cathepsin L inhibitor, 26) a CD40 antagonist, 27) a chemokine receptor agonist, 28) a chymase inhibitor, 29) a collagenase antagonist, 30) a CXCR antagonist, 31) a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), CGP 74514A, bohemine, olomoucine (CAS No. 101622-51-9), indole-3-carbinol (CAS No. 700-06-1), and an analogue or derivative thereof, 32) a cyclooxygenase 1 inhibitor, 33) a DHFR inhibitor, 34) a dual integrin inhibitor, 35) an elastase inhibitor, 36) an elongation factor-1 alpha inhibitor, 37) an endothelial growth factor antagonist, 38) an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), lavendustin A (CAS No. 125697-92-9), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), SU 1498 (a VEGF-R inhibitor), a VEGFR-2 kinase inhibitor (Bristol-Myers Squibb), XL-647 (Exelixis), a KDR inhibitor from Abbott Laboratories, sorafenib tosylate, and an analogue or derivative thereof, 39) an endotoxin antagonist, 40) an epothilone and tubulin binder, 41) an estrogen receptor antagonist, 42) an FGF inhibitor, 43) a farnexyl transferase inhibitor, 44) a farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof, 45) an FLT-3 kinase inhibitor, 46a) an FGF receptor kinase inhibitor, 47) a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), mevastatin, and an analogue or derivative thereof, 48) a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), radicicol from Humicola fuscoatra (CAS No. 12772-57-5), and an analogue or derivative thereof, 49) a histone deacetylase inhibitor, 50) an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), cerivastatin Na (CAS No. 143201-11-0), and an analogue or derivative thereof, 51) an ICAM inhibitor, 52) an IL, ICE and IRAK antagonist, wherein the antagonist is a CJ-14877, CP-424174 (Pfizer), NF-61 (Negma-Lerads), and an analogue or derivative thereof, 53) an IL-2 inhibitor, 54) an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof, 55) an IMPDH (inosine monophosphate), 56) an integrin antagonist, 57) an interleukin antagonist, 58) an inhibitor of type III receptor tyrosine kinase, 59) an irreversible inhibitor of enzyme methionine aminopeptidase type 2, 60) an isozyme selective delta protein kinase C inhibitor, 61) a JAK3 enzyme inhibitor, 62) a JNK inhibitor, 63) a kinase inhibitor, 64) a kinesin antagonist, 65) a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof, 66) a MAP kinase inhibitor, 67) a matrix metalloproteinase inhibitor, 68) an MCP-CCR2 inhibitor, 69) an mTOR inhibitor, 70) an mTOR kinase inhibitor,71) a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, dolastatin 15 (CAS No. 123884-00-4), vincamine, and an analogue or derivative thereof, 72) an MIF inhibitor, 73) an MMP inhibitor, 74) a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof, 75) an NF kappa B inhibitor selected from the group consisting of emodin (CAS No. 518-82-1), AVE-0545 or AVE-0547 (Sanofi-Aventis), bortezomib (CAS No. 179324-69-7) (Millennium Pharmaceuticals), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), Bay 11-7085, and an analogue or derivative thereof, 76) a nitric oxide agonist, 77) an ornithine decarboxylase inhibitor, 78) a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), SKF86002 (CAS No. 72873-74-6), RPR-200765A (Sanofi-Aventis), SD-282 (Johnson & Johnson), TAK-715 (Takeda), and an analogue or derivative thereof, 79) a palmitoyl-protein thioesterase inhibitor, 80) a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof, 81) a peroxisome proliferators-activated receptor agonist selected from the group consisting of (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-154), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), LBM642, WY-14,643 (CAS No. 50892-23-4), GW7647, fenofibric acid (CAS No. 42017-89-0), MCC-555 (CAS No. 161600-01-7), GW9662, GW1929, GW501516, L-165,041 (CAS No. 79558-09-1), and an analogue or derivative thereof, 82) a phosphatase inhibitor, 83) a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-1 4-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), theobromine (CAS No. 83-67-0), papverine hydrochloride (CAS No. 61-25-6), quercetin dehydrate (CAS No. 6151-25-3), YM 976 (CAS No. 191219-80-4), irsogladine (CAS No. 57381-26-7), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof, 84) a PKC inhibitor, 85) a platelet activating factor antagonist, 86) a platelet-derived growth factor receptor kinase inhibitor, 87) a prolyl hydroxylase inhibitor, 88) a polymorphonuclear neutrophil inhibitor, 89) a protein kinase B inhibitor, 90) a protein kinase C stimulant, 91) a purine nucleoside analogue, 92) a purinoreceptor P2X antagonist, 93) a Raf kinase inhibitor, 94) a reversible inhibitor of ErbB1 and ErbB2, 95) a ribonucleoside triphosphate reductase inhibitor, 96) an SDF-1 antagonist, 97) a sheddase inhibitor, 98) an SRC inhibitor, 99) a stromelysin inhibitor, 100) an Syk kinase inhibitor, 101) a telomerase inhibitor, 102) a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof, 103) a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CGx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), iP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof, 104) a tumor necrosis factor antagonist, 105) a Toll receptor inhibitor, 106) a tubulin antagonist, 107) a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 13683148-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), herbimycin A, and an analogue or derivative thereof, 108) a VEGF inhibitor, 109) a vitamin D receptor agonist, 110) ZD-6474 (an angiogenesis inhibitor), 111) AP-23573 (an mTOR inhibitor), 112) synthadotin (a tubulin antagonist), 113) S-0885 (a collagenase inhibitor), 114) aplidine (an elongation factor-1 alpha inhibitor), 115) ixabepilone (an epithilone), 116) IDN-5390 (an angiogenesis inhibitor and an FGF inhibitor), 117) SB-2723005 (an angiogenesis inhibitor), 118) ABT-518 (an angiogenesis inhibitor), 119) combretastatin (an angiogenesis inhibitor), 120) anecortave acetate (an angiogenesis inhibitor), 121) SB-715992 (a kinesin antagonist), 122) temsirolimus (an mTOR inhibitor), and 123) adalimumab (a TNFα antagonist), 124) erucylphosphocholine (an ATK inhibitor), 125) alphastatin (an angiogenesis inhibitor), 126) bortezomib (an NF Kappa B inhibitor), 127) etanercept (a TNFα antagonist and TACE inhibitor), 128) humicade (a TNFα inhibitor), and 129) gefitinib (a tyrosine kinase inhibitor), 130) a histamine receptor antagonist selected from the group consisting of phenothiazines (e.g., promethazine), alkylamines (e.g., chlorpheniramine (CAS No. 7054-11-7), brompheniramine (CAS No. 980-71-2), fexofenadine hydrochloride, promethazine hydrochloride, loratadine, ketotifen fumarate salt, and acrivastine), methylxanthines (e.g., theophylline, theobromine, and caffeine), cimetidine (available under the tradename TAGAMET from SmithKline Beecham Phamaceutical Co., Wilmington, Del.), ranitidine (available under the tradename ZANTAC from Warner Lambert Company, Morris Plains, N.J.), famotidine (available under the tradename PEPCID from Merck & Co., Whitehouse Station, N.J.), nizatidine (available under the tradename AXID from Reliant Pharmaceuticals, Inc., Liberty Corner, N.J.), nizatidine, and roxatidine acetate (CAS No. 78628-28-1), H3 receptor antagonists (e.g., thioperamide and thioperamide maleate salt), and anti-histamines (e.g., tricyclic dibenozoxepins, ethanolamines, ethylenediamines, piperizines, piperidines, and pthalazinones), 131) an alpha adrenergic receptor antagonist, 132) an anti-psychotic compound, 133) a CaM kinase II inhibitor, 134) a G protein agonist, 135) an antibiotic selected from the group consisting of apigenin (Cas No. 520-36-5), ampicillin sodium salt (CAS No. 69-52-3), puromycin, and an analogue or derivative thereof, 136) an anti-microbial agent, 137) a DNA topoisomerase inhibitor selected from the group consisting of β-lapachone (CAS No. 4707-32-8), (−)-arctigenin (CAS No. 7770-78-7), aurintricarboxylic acid, and an analogue or derivative thereof, 138) a thromboxane A2 receptor inhibitor selected from the group consisting of BM-531 (CAS No. 284464-46-6), ozagrel hydrochloride (CAS No. 78712-43-3), and an analogue or derivative thereof, 139) a D2 dopamine receptor antagonist, 140) a Peptidyl-Prolyl Cis/Trans Isomerase Inhibitor, 141) a dopamine antagonist, an anesthetic compound, 142) a clotting factor, 143) a lysyl hydrolase inhibitor, 144) a muscarinic receptor inhibitor, 145) a superoxide anion generator, 146) a steroid, 147) an anti-proliferative agent selected from the group consisting of silibinin (CAS No. 22888-70-6), silymarin (CAS No. 65666-07-1), 1,2-hexanediol, dioctyl phthalate (CAS No. 117-81-7), zirconium (IV) oxide, glycyrrhizic acid, spermidine trihydrochloride, tetrahydrochloride, CGP 74514, spermine tetrahydrochloride, NG-methyl-L-arginine acetate salt, galardin, and an analogue or derivative thereof, 148) a diuretic, 149) an anti-coagulant, 150) a cyclic GMP agonist, 151) an adenylate cyclase agonist, 152) an antioxidant, 153) a nitric oxide synthase inhibitor, 154) an anti-neoplastic agent selected from tirapazamine (CAS No. 27314-97-2), fludarabine (CAS No. 21679-14-1), cladribine, imatinib mesilate, and an analogue or derivative thereof, 155) a DNA synthesis inhibitor, 156) a DNA alkylating agent selected from dacarbazine (CAS No. 4342-03-4), temozolomide, procarbazine HCl, and an analogue or derivative thereof, 157) a DNA methylation inhibitor, 158) a NSAID agent, 159) a peptidylglycine alpha-hydroxylating monooxygenase inhibitor, 160) an MEK1/MEK 2 inhibitor, 161) a NO synthase inhibitor, 162) a retinoic acid receptor antagonist selected from isotretinoin (CAS No. 4759-48-2) and an analogue or derivative thereof, 163) an ACE inhibitor, 164) a glycosylation inhibitor, 165) an intracellular calcium influx inhibitor, 166) an anti-emetic agent, 167) an acetylcholinesterase inhibitor, 168) an ALK-5 receptor antagonist, 169) a RAR/RXT antagonist, 170) an eIF-2a inhibitor, 171) an S-adenosyl-L-homocysteine hydrolase inhibitor, 172) an estrogen agonist, 173) a serotonin receptor inhibitor, 174) an anti-thrombotic agent, 175) a tryptase inhibitor, 176) a pesticide, 177) a bone mineralization promoter, 178) a bisphosphonate compound selected from risedronate and an analogue or derivative thereof, 179) an anti-inflammatory compound, 180) a DNA methylation promoter, 181) an anti-spasmodic agent, 182) a protein synthesis inhibitor, 183) an α-glucosidase inhibitor, 184) a calcium channel blocker, 185) a pyruvate dehydrogenase activator, 186) a prostaglandin inhibitor, 187) a sodium channel inhibitor, 188) a serine protease inhibitor, 189) an intracellular calcium flux inhibitor, 190) a JAK2 inhibitor; 191) an androgen inhibitor, 192) an aromatase inhibitor, 193) an anti-viral agent, 194) a 5-HT inhibitor, 195) an FXR antagonist, 196) an actin polymerization and stabilization promoter, 197) an AXOR12 agonist, 198) an angiotensin II receptor agonist, 199) a platelet aggregation inhibitor, 200) a CB1/CB2 receptor agonist, 201) a norepinephrine reuptake inhibitor, 202) a selective serotonin reuptake inhibitor, 203) a reducing agent, 204) Isotretinoin, 205) radicicol, 206) clobetasol propionate, 207) homoharringtonine, 208) trichostatin A, 209) brefeldin A, 210) thapsigargin, 211) dolastatin 15, 212) cerivastatin, 213) jasplakinolide, 214) herbimycin A, 215) pirfenidone, 216) vinorelbine, 217) 17-DMAG, 218) tacrolimus, 219) loteprednol etabonate, 220) juglone, 221) prednisolone, 222) puromycin, 223) 3-BAABE, 224) cladribine, 225) mannose-6-phosphate, 226) 5-azacytidine, 227) Ly333531 (ruboxistaurin), 228) simvastatin, and 229) an immuno-modulator selected from Bay 11-7085, (−)-arctigenin, idazoxan hydrochloride, and an analogue or derivative thereof.

Provided below are exemplary dosage ranges for a variety of anti-scarring agents which can be used in conjunction with electrical devices in accordance with the invention. (A) Angiogenesis inhibitors including alphastatin, ZD-6474, IDN-5390, SB-2723005, ABT-518, combretastatin, and anecortane, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (B) mTOR inhibitors including AP-23573 and temsirolimus, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (C) Tubulin antagonists including synthadotin, analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (D) Epithilones including ixabepilone and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (E) Kinesin Antagonists including SB-715992 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (F) TNF alpha antagonists including etanercept, humicade, adalimumab and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (G) AKT inhibitor including erucylphosphocholine and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (H) FGF Inhibitors including IDN-5390 and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (I) Collagenase Antagonists including S-0885 and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mm²-100 μg/mm². Minimum concentration of 10⁻⁸-10⁻⁴ M of agent is to be maintained on the implant or barrier surface. (J) NF KAPPA B Inhibitors including bortezomib and analogues and derivatives thereof: total dose not to exceed 200 mg (range of 0.1 μg to 200 mg); preferred 1 μg to 100 mg. Dose per unit area of 0.01 μg-100 μg per mm²; preferred dose of 0.1 μg/mm²-20 μg/mm². Minimum concentration of 10⁻⁸-10⁻⁴ M of agent is to be maintained on the implant or barrier surface. (K) Elongation Factor-1 alpha inhibitors including aplidine and analogues and derivatives thereof: total dose not to exceed 1000 mg (range of 0.1 μg to 1000 mg); preferred 1 μg to 500 mg. Dose per unit area of 0.01 μg-500 μg per mm²; preferred dose of 0.1 μg/mM²-100 μg/mm². Minimum concentration of 10⁻⁸-10⁻⁴ M of agent is to be maintained on the implant or barrier surface. (L) Tyrosine kinase inhibitors including gefitinib and analogues and derivatives thereof: total dose not to exceed 1000 mg (range of 0.1 μg to 1000 mg); preferred 1 μg to 500 mg. Dose per unit area of 0.01 μg-500 μg per mm² preferred dose of 0.1 μg/mm²-100 μg/mm². Minimum concentration of 10⁻⁸-10⁻⁴ M of agent is to be maintained on the implant or barrier surface.

Additional examples of anti-scarring agents which can be used include those having a high potency in the assays described herein (approximately 1-100 nM IC₅₀ range) such as isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, and tacrolimus. For high potency drugs, the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁻⁴ M of agent should be maintained on the implant or barrier surface.

Other examples of agents which can be used include those having a mid-potency in the assays described herein (approximately 100-500 nM IC₅₀ range) such as loteprednol etabonate, juglone, prednisolone, puromycin, 3-BAABE, cladribine, and mannose-6-phosphate. For mid-potency drugs, the total dose typically should not to exceed 500 mg (range of 1 0 μg to 500 mg) and preferably 1 μg to 200 mg; dose per unit area of 0.01 μg-200 μg per mm², preferably 0.1 μg/mm²-40 μg/mm²; and minimum concentration of 10⁻⁸-10⁻⁴ M of agent should be maintained on the implant or barrier surface.

Other examples of agents which can be used include those having a low potency in the assays described herein (approximately 500-1000 nm range IC₅₀ range) such as 5-azacytidine, Ly333531 (ruboxistaurin), and simvastatin. For low potency drugs, the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm²; and minimum concentration of 10⁻⁸-10⁻⁴ M of agent should to be maintained on the implant or barrier surface.

D. Delivery of Therapeutic Agents or Compositions and Generating Electical Devices that Comprise Therapeutic Agents or Compositions

In the practice of this invention, drug-coated or drug-impregnated implants and medical devices are provided which inhibit fibrosis (or gliosis) in and around the device, lead and/or electrode of neurostimulation or cardiac rhythm management (CRM) devices. Within various embodiments, fibrosis (or gliosis) is inhibited by local, regional or systemic release of specific pharmacological agents that become localized to the tissue adjacent to the device or implant. There are numerous neurostimulation and CRM devices where the occurrence of a fibrotic (or gliotic) reaction may adversely affect the functioning of the device or the biological problem for which the device was implanted or used. Typically, fibrotic (or gliotic) encapsulation of the electrical lead (or the growth of fibrous/glial tissue between the lead and the target nerve tissue) slows, impairs, or interrupts electrical transmission of the impulse from the device to the tissue. This can cause the device to function suboptimally or not at all, or can cause excessive drain on battery life as increased energy is required to overcome the electrical resistance imposed by the intervening scar (or glial) tissue. There are numerous methods available for optimizing delivery of the fibrosis-inhibiting (or gliosis-inhibiting) agent to the site of the intervention and several of these are described below.

1) Delivery of Therapeutic Agents Via Electrical Devices and Generating Electical Devices that Comprise Therapeutic Agents

Medical devices or implants of the present invention are coated with, or adapted to release an agent which inhibits fibrosis (or gliosis) on the surface of, or around, the neurostimulator or CRM device, lead and/or electrode. In one aspect, the present invention provides electrical devices that include an anti-scarring (or anti-gliotic) agent or a composition that includes an anti-scarring (or anti-gliotic) agent such that the overgrowth of granulation (or gliotic) tissue is inhibited or reduced.

Methods for incorporating fibrosis-inhibiting (or gliosis-inhibiting) compositions onto or into CRM or neurostimulator devices include: (a) directly affixing to the device, lead and/or the electrode a fibrosis-inhibiting (or gliosis-inhibiting) composition (e.g., by either a spraying process or dipping process as described above, with or without a carrier), (b) directly incorporating into the device, lead and/or the electrode a fibrosis-inhibiting (or gliosis-inhibiting) composition (e.g., by either a spraying process or dipping process as described above, with or without a carrier (c) by coating the device, lead and/or the electrode with a substance such as a hydrogel which may in turn absorb the fibrosis-inhibiting (or gliosis-inhibiting) composition, (d) by interweaving fibrosis-inhibiting (or gliosis-inhibiting) composition coated thread (or the polymer itself formed into a thread) into the device, lead and/or electrode structure, (e) by inserting the device, lead and/or the electrode into a sleeve or mesh which is comprised of, or coated with, a fibrosis-inhibiting (or gliosis-inhibiting) composition, (f) constructing the device, lead and/or the electrode itself (or a portion of the device and/or the electrode) with a fibrosis-inhibiting (or gliosis-inhibiting) composition, or (g) by covalently binding the fibrosis-inhibiting (or gliosis-inhibiting) agent directly to the device, lead and/or electrode surface or to a linker (small molecule or polymer) that is coated or attached to the device surface. For these devices, leads and electrodes, the coating process can be performed in such a manner as to: (a) coat the non-electrode portions of the lead or device; (b) coat the electrode portion of the lead; (c) coat the sensor part of the lead; or (d) coat all or parts of the entire device with the fibrosis-inhibiting (or gliosis-inhibiting) composition. In addition to, or alternatively, the fibrosis-inhibiting (or gliosis-inhibiting) agent can be mixed with the materials that are used to make the device, lead and/or electrode such that the fibrosis-inhibiting agent is incorporated into the final product.

In addition to, or as an alternative to incorporating a fibrosis-inhibiting (or gliosis-inhibiting) agent onto or into the CRM or neurostimulation device, the fibrosis-inhibiting (or gliosis-inhibiting) agent can be applied directly or indirectly to the tissue adjacent to the CRM or neurostimulator device (preferably near the electrode-tissue interface). This can be accomplished by applying the fibrosis-inhibiting (or gliosis inhibiting) agent, with or without a polymeric, non-polymeric, or secondary carrier: (a) to the lead and/or electrode surface (e.g., as an injectable, paste, gel or mesh) during the implantation procedure); (b) to the surface of the tissue (e.g., as an injectable, paste, gel, in situ forming gel or mesh) prior to, immediately prior to, or during, implantation of the CRM or neurostimulation device, lead and/or electrode; (c) to the surface of the lead and/or electrode and/or the tissue surrounding the implanted lead and/or electrode (e.g., as an injectable, paste, gel, in situ forming gel or mesh) immediately after to the implantation of the CRM or neurostimulation device, lead and/or electrode; (d) by topical application of the anti-fibrosis (or gliosis) agent into the anatomical space where the CRM or neurostimulation device, lead and/or electrode may be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device may be inserted); (e) via percutaneous injection into the tissue surrounding the device, lead and/or electrode as a solution as an infusate or as a sustained release preparation; (f) by any combination of the aforementioned methods. Combination therapies (i.e., combinations of therapeutic agents and combinations with antithrombotic and/or antiplatelet agents) can also be used.

In another embodiment, the anti-fibrosing (or gliosis-inhibiting) agent can be coated onto the entire device or a portion of the device. In certain embodiments, the agent is present as part of a coating on a surface of the CRM or neurostimulation device, lead and/or electrode. The coating may partially cover or may completely cover the surface of the electrical device, lead and/or electrode. Further, the coating may directly or indirectly contact the electrical device, lead and/or electrode. For example, the CRM or neurostimulation device, lead and/or electrode may be coated with a first coating and then coated with a second coating that includes the anti-scarring (or gliosis-inhibiting) agent.

CRM and neurostimulation devices, leads and/or electrodes may be coated using a variety of coating methods, including by dipping, spraying, painting, by vacuum deposition, or by any other method known to those of ordinary skill in the art.

As described above, the anti-fibrosing (or anti-gliotic) agent can be coated onto the appropriate CRM or neurostimulation device, lead and/or electrode using the polymeric coatings described above. In addition to the coating compositions and methods described above, there are various other coating compositions and methods that are known in the art. Representative examples of these coating compositions and methods are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

In yet another aspect, anti-scarring (or anti-gliosis) agent may be located within pores or voids of the electrical device, lead and/or electrode. For example, a CRM or neurostimulation device, lead and/or electrode may be constructed to have cavities (e.g., divets or holes), grooves, lumen(s), pores, channels, and the like, which form voids or pores in the body of the device, lead and/or electrode. These voids may be filled (partially or completely) with a fibrosis-inhibiting (or gliosis-inhibiting) agent or a composition that comprises a fibrosis-inhibiting (or gliosis-inhibiting) agent.

Within another aspect of the invention, the biologically active agent can be delivered with non-polymeric agents. These non-polymeric agents can include sucrose derivatives (e.g., sucrose acetate isobutyrate, sucrose oleate), sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites, and combinations and mixtures thereof.

Representative examples of patents relating to non-polymeric delivery systems and their preparation include U.S. Pat. Nos. 5,736,152; 5,888,533; 6,120,789; 5,968,542; and 5,747,058.

The fibrosis-inhibiting (or gliosis-inhibiting) agent may be delivered as a solution. The fibrosis-inhibiting (or gliosis-inhibiting) agent can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, CMC, and the like). In another aspect of the invention, the solution can include a biocompatible solvent, such as ethanol, DMSO, glycerol, PEG-200, PEG-300 or NMP.

Within another aspect of the invention, the fibrosis-inhibiting (or gliosis-inhibiting) agent can further comprise a secondary carrier. The secondary carrier can be in the form of microspheres (e.g., PLGA, PLLA, PDLLA, PCL, gelatin, polydioxanone, poly(alkylcyanoacrylate), nanospheres (e.g., PLGA, PLLA, PDLLA, PCL, gelatin, polydioxanone, poly(alkylcyanoacrylate)), liposomes, emulsions, microemulsions, micelles (e.g., SDS, block copolymers of the form X—Y, X—Y—X or Y—X—Y where X is a poly(alkylene oxide) or alkyl ether thereof (e.g., poly(ethylene glycol), methoxy poly(ethylene glycol), poly(propylene glycol), block copolymers of poly(ethylene oxide) and poly(propylene oxide) [e.g., PLURONIC and PLURONIC R polymers (BASF)]) and Y is a polyester where the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLLA, PDLLA, PCL polydioxanone)), zeolites or cyclodextrins.

Within another aspect of the invention, these fibrosis-inhibiting (or gliosis-inhibiting) agent/secondary carrier compositions can be a) incorporated directly into, or onto, the CRM or neurostimulation device, lead and/or electrode, b) incorporated into a solution, c) incorporated into a gel or viscous solution, d) incorporated into the composition used for coating the device, lead and/or electrode, or e) incorporated into, or onto, the device, lead and/or electrode following coating of the device, lead and/or electrode with a coating composition.

For example, fibrosis-inhibiting (or gliosis-inhibiting) agent loaded PLGA microspheres may be incorporated into a polyurethane coating solution which is then coated onto the device, lead and/or electrode.

In yet another example, the device, lead and/or electrode can be coated with a polyurethane and then allowed to partially dry such that the surface is still tacky. A particulate form of the fibrosis-inhibiting (or gliosis-inhibiting) agent or fibrosis-inhibiting (or gliosis-inhibiting) agent/secondary carrier can then be applied to all or a portion of the tacky coating after which the device is dried.

In yet another example, the device, lead and/or electrode can be coated with one of the coatings described above. A thermal treatment process can then be used to soften the coating, after which the fibrosis-inhibiting (or gliosis-inhibiting) agent or the fibrosis-inhibiting (or gliosis-inhibiting) agent/secondary carrier is applied to the entire device, lead and/or electrode or to a portion of the device, lead and/or electrode (e.g., outer surface).

Within another aspect of the invention, the coated CRM or neurostimulation device, lead and/or electrode which inhibits or reduces an in vivo fibrotic (or gliotic) reaction is further coated with a compound or compositions which delay the release of and/or activity of the fibrosis-inhibiting (or gliosis-inhibiting) agent. Representative examples of such agents include biologically inert materials such as gelatin, PLGA/MePEG film, PLA, polyurethanes, silicone rubbers, surfactants, lipids, or polyethylene glycol, as well as biologically active materials such as heparin or heparin quaternary amine complexes (e.g., heparin-benzalkonium chloride complex) (e.g., to induce coagulation).

For example, in one embodiment of the invention the active agent on the device, lead and/or electrode is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the blood or body fluids, the MePEG may dissolve out of the PLGA, leaving channels through the PLGA to an underlying layer containing the fibrosis-inhibiting (or gliosis-inhibiting) agent, which then can then diffuse into the tissue and initiate its biological activity.

In another embodiment of the invention, for example, a particulate form of the active agent may be coated onto the CRM or neurostimulation device, lead and/or electrode using a polymer (e.g., PLG, PLA, polyurethane) A second polymer that dissolves slowly or degrades (e.g., MePEG-PLGA or PLG) and that does not contain the active agent may be coated over the first layer. Once the top layer dissolves or degrades, it exposes the under coating which allows the active agent to be exposed to the treatment site or to be released from the coating.

Within another aspect of the invention, the outer layer of the coating of a coated CRM or neurostimulation device, lead and/or electrode which inhibits an in vivo fibrotic (or gliotic) response is further treated to crosslink the outer layer of the coating. This can be accomplished by subjecting the coated device, lead and/or electrode to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the CRM or neurostimulator device, lead and/or electrode surface with an inert molecule that prevents access to the active site through steric hindrance, or by coating the surface with an inactive form of the fibrosis-inhibiting (or gliosis-inhibiting) agent, which is later activated. For example, the device, lead and/or electrode can be coated with an enzyme, which causes either release of the fibrosis-inhibiting (or gliosis-inhibiting) agent or activates the fibrosis-inhibiting (or gliosis-inhibiting) agent.

Another example of a suitable CRM or neurostimulation device, lead and/or electrode surface coating includes an anticoagulant such as heparin or heparin quaternary amine complexes (e.g., heparin-benzalkonium chloride complex), which can be coated on top of the fibrosis-inhibiting (or gliosis-inhibiting) agent; this may also be useful during transvenous placement of pacemaker or ICD leads to prevent clotting. The presence of the anticoagulant delays coagulation. As the anticoagulant dissolves away, the anticoagulant activity may stop, and the newly exposed fibrosis-inhibiting (or gliosis-inhibiting) agent may inhibit or reduce fibrosis (or gliosis) from occurring in the adjacent tissue or coating the device, lead and/or electrode.

In another aspect, the CRM or neurostimulation device, lead and/or electrode can be coated with an inactive form of the fibrosis-inhibiting (or gliosis-inhibiting) agent, which is then activated once the device is deployed. Such activation may be achieved by injecting another material into the treatment area after the device, lead and/or electrode (as described below) is implanted or after the fibrosis-inhibiting (or gliosis-inhibiting) agent has been administered to the treatment area (via injections, spray, wash, drug delivery catheters or balloons). In this aspect, the device, lead and/or electrode may be coated with an inactive form of the fibrosis-inhibiting (or gliosis-inhibiting) agent. Once the device, lead and/or electrode is implanted, the activating substance is injected or applied into, or onto, the treatment site where the inactive form of the fibrosis-inhibiting (or gliosis-inhibiting) agent has been applied.

One example of this method includes coating a CRM or neurostimulation device, lead and/or electrode with a biologically active fibrosis-(or gliosis-inhibiting) inhibiting agent, as described herein as described herein. The coating containing the active fibrosis-inhibiting (or gliosis-inhibiting) agent may then be covered with polyethylene glycol and these two substances may then be bonded through an ester bond using a condensation reaction. Prior to the deployment of the device, lead and/or electrode, an esterase is injected into the tissue around the outside of the device (lead or electrode), which can cleave the bond between the ester and the fibrosis-inhibiting (or gliosis-inhibiting) therapeutic agent, allowing the agent to initiate fibrosis (or gliosis) inhibition.

The devices and compositions of the invention may include one or more additional ingredients and/or therapeutic agents, such as surfactants (e.g., PLURONICS, such as F-127, L-122, L-101, L-92, L-81, and L-61), anti-inflammatory agents (e.g., dexamethasone or aspirin), anti-thrombotic agents (e.g., heparin, high activity heparin, heparin quaternary amine complexes (e.g., heparin benzalkonium chloride complex)), anti-infective agents (e.g., 5-fluorouracil, triclosan, rifamycim, and silver compounds), preservatives, anti-oxidants and/or anti-platelet agents.

Within certain embodiments of the invention, the device or therapeutic composition can also comprise radio-opaque, echogenic materials and magnetic resonance imaging (MRI) responsive materials (i.e., MRI contrast agents) to aid in visualization of the composition under ultrasound, fluoroscopy and/or MRI. For example, a composition may be echogenic or radiopaque (e.g., made with echogenic or radiopaque with materials such as powdered tantalum, tungsten, barium carbonate, bismuth oxide, barium sulfate, metrazimide, iopamidol, iohexyl, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). For visualization under MRI, contrast agents (e.g., gadolinium (III) chelates or iron oxide compounds) may be incorporated into the composition. In some embodiments, a medical device may include radio-opaque or MRI visible markers (e.g., bands) that may be used to orient and guide the device during the implantation procedure.

The devices may, alternatively, or in addition, be visualized under visible light, using fluorescence, or by other spectroscopic means. Visualization agents that can be included for this purpose include dyes, pigments, and other colored agents. In one aspect, the composition may further include a colorant to improve visualization of the composition in vivo and/or ex vivo. Frequently, compositions can be difficult to visualize upon delivery into a host, especially at the margins of an implant or tissue. A coloring agent can be incorporated into a composition to reduce or eliminate the incidence or severity of this problem. The coloring agent provides a unique color, increased contrast, or unique fluorescence characteristics to the composition. In one aspect, a composition is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan.

In orie aspect, the devices and compositions of the present invention include one or more coloring agents, also referred to as dyestuffs, which may be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the devices compositions of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the devices and compositions of the present invention include one or more antioxidants, present in an effective amount. Examples of the antioxidant include sulfites, alpha-tocopherol and ascorbic acid.

Within certain aspects of the present invention, the therapeutic composition should be biocompatible, and release one or more fibrosis-inhibiting agents over a period of several hours, days, or, months. As described above, “release of an agent” refers to any statistically significant presence of the agent, or a subcomponent thereof, which has disassociated from the compositions and/or remains active on the surface of (or within) the composition. The compositions of the present invention may release the anti-scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to reduce or inhibit any one or more components of fibrosis (or scarring) (or gliosis), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing an anti-scarring agent at a time such that at least one of the components of fibrosis (or gliosis) is inhibited or reduced. Moreover, the predetermined release rate may reduce agent loading and/or concentration as well as potentially providing minimal drug washout and thus, increases efficiency of drug effect. Any one of the anti-scarring agents described herein may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, and substantially non-releasing.

The total amount of anti-scarring agent made available on, in or near the device may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The surface amount of anti-scarring agent on, in or near the device may be in an amount ranging from less than 0.01 μg to about 250 μg per mm² of device surface area. Generally, the anti-scarring agent may be in the amount ranging from less than 0.01 μg per mm²; or from 0.01 μg to about 10 μg per mm²; or from 10 μg to about 250 μg per mm².

The anti-scarring agent that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring agent released from the composition as a function of time may be determined based on the in vitro release characteristics of the agent from the composition. The in vitro release rate may be determined by placing the anti-scarring agent within the composition or device in an appropriate buffer such as 0.1M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from an amount ranging from about 0.01 μg (micrograms) to about 2500 mg (milligrams). Generally, the anti-scarring agent that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring agent is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

Further, therapeutic compositions and devices of the present invention should preferably have a stable shelf-life of at least several months and be capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In another aspect, the compositions and devices of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics. In another embodiment, a secondary package can be used for the product. In another embodiment, product can be in a sterile container that is placed in a box that is labeled to describe the contents of the box.

Coating of CRM or Neurostimulation Devices, Leads and Electrodes with Fibrosis-Inhibiting (or Gliosis-Inhibiting) Agents

As described above, a range of polymeric and non-polymeric materials can be used to incorporate the fibrosis-inhibiting (or gliosis-inhibiting) agent onto or into an electrical device, lead or electrode. Coating the device, lead and/or electrode with these fibrosis-inhibiting (or gliosis-inhibiting) agent-containing compositions, or with the fibrosis-inhibiting (or gliosis-inhibiting) agent only, is one process that can be used to incorporate the fibrosis-inhibiting (or gliosis-inhibiting) agent into or onto the device, lead and/or electrode.

a) Dip Coating

Dip coating is an example of coating process that can be used to associate the anti-scarring (or gliosis-inhibiting) agent with the device, lead and/or electrode. In one embodiment, the fibrosis-inhibiting (or gliosis-inhibiting) agent is dissolved in a solvent for the fibrosis-inhibiting (or gliosis-inhibiting) agent and is then coated onto the device, lead and/or electrode.

Fibrosis-Inhibiting (or Gliosis-Inhibiting) Agent with an Inert Solvent

In one embodiment, the solvent is an inert solvent for the device, lead or electrode such that the solvent does not dissolve the medical device, lead or electrode to any great extent and is not absorbed by the device, lead or electrode to any great extent. The device, lead or electrode can be immersed, either partially or completely, in the fibrosis-inhibiting (or gliosis-inhibiting) agent/solvent solution for a specific period of time. The rate of immersion into the fibrosis-inhibiting (or gliosis-inhibiting) agent/solvent solution can be altered (e.g., 0.001 cm per sec to 50 cm per sec). The device, lead and/or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application, where higher repetitions generally increase the amount of agent that is coated onto the device, lead or electrode. The device, lead or electrode can be dried under vacuum to reduce residual solvent levels. This process may result in the fibrosis-inhibiting (or gliosis-inhibiting) agent being coated on the surface of the device.

Fibrosis-Inhibiting (or Gliosis-Inhibiting) Agent with a Swelling Solvent

In one embodiment, the solvent is one that will not dissolve the CRM or neurostimulation device, lead or electrode but will be absorbed by the device, lead or electrode. In certain cases, these solvents can swell the device, lead or electrode to some extent. The device, lead or electrode can be immersed, either partially or completely, in the fibrosis-inhibiting (or gliosis-inhibiting) agent/solvent solution for a specific period of time (seconds to days). The rate of immersion into the fibrosis-inhibiting (or gliosis-inhibiting) agent/solvent solution can be altered (e.g., 0.001 cm per sec to 50 cm per sec). The device, lead and/or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process results in the fibrosis-inhibiting (or gliosis-inhibiting) agent being adsorbed into the CRM or neurostimulation device, lead or electrode. The fibrosis-inhibiting (or gliosis-inhibiting) agent may also be present on the surface of the device, lead and/or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent, or by spraying the coated device, lead or electrode with a solvent for the fibrosis-inhibiting (or gliosis-inhibiting) agent.

Fibrosis-Inhibiting (or Gliosis-Inhibiting) Agent with a Solvent

In one embodiment, the solvent is one that may be absorbed by the device, lead or electrode and that will dissolve the device, lead or electrode. The device, lead or electrode can be immersed, either partially or completely, in the fibrosis-inhibiting (or gliosis-inhibiting) agent/solvent solution for a specific period of time (seconds to hours). The rate of immersion into the fibrosis-inhibiting (or gliosis-inhibiting) agent/solvent solution can be altered (e.g., 0.001 cm per sec to 50 cm per sec). The device, lead or electrode can then be removed from the solution. The rate at which the device, lead or electrode is withdrawn from the solution can be altered (e.g., 0.001 cm per sec to 50 cm per sec). The coated device, lead or electrode can be air-dried. The dipping process can be repeated one or more times depending on the specific application. The device, lead or electrode can be dried under vacuum to reduce residual solvent levels. This process will result in the fibrosis-inhibiting (or gliosis-inhibiting) agent being adsorbed into the medical device, lead or electrode as well as being surface associated. The exposure time of the device, lead or electrode to the solvent should not incur significant permanent dimensional changes to the device, lead or electrode. The fibrosis-inhibiting (or gliosis-inhibiting) agent may also be present on the surface of the device, lead or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent or by spraying the coated device, lead or electrode with a solvent for the fibrosis-inhibiting (or gliosis-inhibiting) agent.

In one embodiment, the fibrosis-inhibiting (or gliosis-inhibiting) agent and a polymer are dissolved in a solvent, for both the polymer and the fibrosis-inhibiting (or gliosis-inhibiting) agent, and are then coated onto the device, lead or electrode.

In the above description the device, lead or electrode can be one that has not been modified or one that has been further modified by coating with a polymer, surface treated by plasma treatment, flame treatment, corona treatment, surface oxidation or reduction, surface etching, mechanical smoothing or roughening, or grafting prior to the coating process.

In any one the above dip coating methods, the surface of the device, lead or electrode can be treated with a plasma polymerization method prior to coating of the fibrosis-inhibiting (or gliosis-inhibiting) agent or fibrosis-inhibiting (or gliosis-inhibiting) agent-containing composition, such that a thin polymeric layer is deposited onto the device, lead or electrode surface. Examples of such methods include parylene coating of devices and the use of various monomers such hydrocyclosiloxane monomers. Parylene coating may be especially advantageous if the device, or portions of the device (such as the lead or the electrode), are composed of materials (e.g., stainless steel, nitinol) that do not allow incorporation of the therapeutic agent(s) into the surface layer using one of the above methods. A parylene primer layer may be deposited onto the electrical device, lead or electrode using a parylene coater (e.g., PDS 2010 LABCOATER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and Parylene D, a dichlorinated derivative of PARYLENE N).

b) Spray Coating CRM and Neurostimulation Devices, Leads and Electrodes

Spray coating is another coating process that can be used. In the spray coating process, a solution or suspension of the fibrosis-inhibiting (or gliosis-inhibiting) agent, with or without a polymeric or non-polymeric carrier, is nebulized and directed to the device, lead and/or electrode to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting (or gliosis-inhibiting) agent is dissolved in a solvent for the fibrosis agent and is then sprayed onto the device, lead and/or electrode.

Fibrosis-Inhibiting (or Gliosis-Inhibiting) Agent with an Inert Solvent

In one embodiment, the solvent is an inert solvent for the device, lead or electrode such that the solvent does not dissolve the medical device, lead or electrode to any great extent and is not absorbed to any great extent. The device, lead or electrode can be held in place or mounted onto a mandrel or rod that has the ability to move in an X, Y or Z plane or a combination of these planes. Using one of the above described spray devices, the device, lead or electrode can be spray coated such that it is either partially or completely coated with the fibrosis-inhibiting (or gliosis-inhibiting) agent/solvent solution. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. This process results in the fibrosis-inhibiting (or gliosis-inhibiting) agent being coated on the surface of the device, lead and/or electrode.

Fibrosis-Inhibiting (or Gliosis-Inhibiting) Agent with a Swelling solvent

In one embodiment, the solvent is one that will not dissolve the device, lead or electrode but will be absorbed by it. These solvents can thus swell the device, lead or electrode to some extent. The device, lead or electrode can be spray coated, either partially or completely, in the fibrosis-inhibiting (or gliosis-inhibiting) agent/solvent solution. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. This process can result in the fibrosis-inhibiting (or gliosis-inhibiting) agent being adsorbed into the medical device, lead or electrode. The fibrosis-inhibiting (or gliosis-inhibiting) agent may also be present on the surface of the device, lead or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent, or by spraying the coated device, lead or electrode with a solvent for the fibrosis-inhibiting (or gliosis-inhibiting) agent.

Fibrosis-Inhibiting (or Gliosis-Inhibiting) Agent with a Solvent

In one embodiment, the solvent is one that will be absorbed by the device, lead or electrode and that will dissolve it. The device, lead or electrode can be spray coated, either partially or completely, in the fibrosis-inhibiting (or gliosis-inhibiting) agent/solvent solution. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. This process will result in the fibrosis-inhibiting (or gliosis-inhibiting) agent being adsorbed into the medical device, lead or electrode as well as being surface associated. In one embodiment, the exposure time of the device, lead or electrode to the solvent may not incur significant permanent dimensional changes to it. The fibrosis-inhibiting (or gliosis-inhibiting) agent may also be present on the surface of the device, lead or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent, or by spraying the coated device, lead or electrode with a solvent for the fibrosis-inhibiting (or gliosis-inhibiting) agent.

In the above description the device, lead or electrode can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosis-inhibiting (or gliosis-inhibiting) agent and a polymer are dissolved in a solvent, for both the polymer and the anti-fibrosing (or gliosis-inhibiting) agent, and are then spray coated onto the device, lead or electrode.

Fibrosis-Inhibiting (or Gliosis-Inhibiting) Agent/Polymer with an Inert Solvent

In one embodiment, the solvent is an inert solvent for the device, lead or electrode such that the solvent does not dissolve it to any great extent and is not absorbed by it to any great extent. The device, lead or electrode can be spray coated, either partially or completely, in the fibrosis-inhibiting (or gliosis-inhibiting) agent/polymer/solvent solution for a specific period of time. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. This process can result in the fibrosis-inhibiting (or gliosis-inhibiting) agent/polymer being coated on the surface of the device, lead or electrode.

Fibrosis-Inhibiting (or Gliosis-Inhibiting) Agent/Polymer with a Swelling Solvent

In one embodiment, the solvent is one that will not dissolve the device, lead or electrode but will be absorbed by it. These solvents can thus swell the device, lead or electrode to some extent. The device, lead or electrode can be spray coated, either partially or completely, in the fibrosis-inhibiting (or gliosis-inhibiting) agent/polymer/solvent solution. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. This process will result in the fibrosis-inhibiting (or gliosis-inhibiting) agent/polymer being coated onto the surface of the device, lead or electrode as well as the potential for the fibrosis-inhibiting (or gliosis-inhibiting) agent being adsorbed into the medical device, lead or electrode. The fibrosis-inhibiting (or gliosis-inhibiting) agent may also be present on the surface of the device, lead or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent or by spraying the coated device, lead or electrode with a solvent for the fibrosis-inhibiting (or gliosis-inhibiting) agent.

Fibrosis-Inhibiting (or Gliosis-Inhibiting) Agent/Polymer with a Solvent

In one embodiment, the solvent is one that will be absorbed by the device, lead or electrode and that will dissolve it. The device, lead or electrode can be spray coated, either partially or completely, in the fibrosis-inhibiting (or gliosis-inhibiting) agent/solvent solution. The rate of spraying of the fibrosis-inhibiting (or gliosis-inhibiting) agent/solvent solution can be altered (e.g., 0.001 mL per sec to 10 mL per sec) to ensure that a good coating of the fibrosis-inhibiting (or gliosis-inhibiting) agent is obtained. The coated device, lead or electrode can be air-dried. The spray coating process can be repeated one or more times depending on the specific application. The device, lead or electrode can be dried under vacuum to reduce residual solvent levels. In the preferred embodiment, the exposure time of the device, lead or electrode to the solvent may not incur significant permanent dimensional changes to it (other than those associated with the coating itself). The fibrosis-inhibiting (or gliosis-inhibiting) agent may also be present on the surface of the device, lead or electrode. The amount of surface associated fibrosis-inhibiting (or gliosis-inhibiting) agent may be reduced by dipping the coated device, lead or electrode into a solvent for the fibrosis-inhibiting (or gliosis-inhibiting) agent or by spraying the coated device, lead or electrode with a solvent for the fibrosis-inhibiting (or gliosis-inhibiting) agent.

In the above description the device, lead or electrode can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting (or gliosis-inhibiting) agent in a polymer solution can be prepared. The suspension can be prepared by choosing a solvent that can dissolve the polymer but not the fibrosis-inhibiting (or gliosis-inhibiting) agent, or a solvent that can dissolve the polymer and in which the fibrosis-inhibiting (or gliosis-inhibiting) agent is above its solubility limit. In similar processes described above, the suspension of the fibrosis-inhibiting (or gliosis-inhibiting) and polymer solution can be sprayed onto the CRM or neurostimulation device, lead or electrode such that it is coated with a polymer that has a fibrosis-inhibiting (or gliosis-inhibiting) agent suspended within it.

2) Systemic, Regional and Local Delivery of Fibrosis-Inhibiting (or Gliosis-Inhibiting) Agents

A variety of drug-delivery technologies are available for systemic, regional and local delivery of therapeutic agents. Several of these techniques may be suitable to achieve preferentially elevated levels of fibrosis-inhibiting (or gliosis-inhibiting) agents in the vicinity of the CRM or neurostimulation device, lead and/or electrode, including: (a) using drug-delivery catheters for local, regional or systemic delivery of fibrosis-inhibiting (or gliosis-inhibiting) agents to the tissue surrounding the device or implant. Typically, drug delivery catheters are advanced through the circulation or inserted directly into tissues under radiological guidance until they reach the desired anatomical location. The fibrosis inhibiting agent can then be released from the catheter lumen in high local concentrations in order to deliver therapeutic doses of the drug to the tissue surrounding the device or implant; (b) drug localization techniques such as magnetic, ultrasonic or MRI-guided drug delivery; (c) chemical modification of the fibrosis-inhibiting (or gliosis-inhibiting) drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inhibiting (or gliosis-inhibiting) drug or formulation designed to localize the drug to areas of bleeding or disrupted vasculature; and/or (e) direct injection of the fibrosis-inhibiting (or gliosis-inhibiting) agent, for example, under endoscopic vision.

3) Infiltration of Fibrosis-Inhibiting (or Gliosis-Inhibiting) Agents into the Tissue Surrounding a Device or Implant

Alternatively, the tissue surrounding the CRM or neurostimulation device can be treated with a fibrosis-inhibiting (or gliosis-inhibiting) agent or a composition that comprises a fibrosis-inhibiting (or gliosis-inhibiting) agent prior to, during, or after the implantation procedure. A fibrosis-inhibiting (or gliosis-inhibiting) agent or a composition comprising a fibrosis-inhibiting (or gliosis-inhibiting) agent may be infiltrated around the device or implant by applying the composition directly and/or indirectly into and/or onto (a) tissue adjacent to the medical device; (b) the vicinity of the medical device-tissue interface; (c) the region around the medical device; and (d) tissue surrounding the medical device.

Methods for infiltrating the subject compositions into tissue adjacent to a medical device include delivering the fibrosis-inhibiting (or gliosis-inhibiting) agent composition: (a) to the medical device surface (e.g., as an injectable, paste, gel or mesh) during the implantation procedure; (b) to the surface of the tissue (e.g., as an injectable, paste, gel, in situ forming gel or mesh) immediately prior to, or during, implantation of the medical device; (c) to the surface of the medical device and/or the tissue surrounding the implanted medical device (e.g., as an injectable, paste, gel, in situ forming gel or mesh) immediately after the implantation of the medical device; (d) by topical application of the composition into the anatomical space where the medical device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the medical device as a solution as an infusate or as a sustained release preparation; (f) by any combination of the aforementioned methods. Combination therapies (e.g., combinations of therapeutic agents and combinations with antithrombotic and/or antiplatelet agents) may also be used. In all cases it is understood that the subject compositions may be infiltrated into tissue adjacent to all or a portion of the device.

It should be noted that certain polymeric carriers themselves can help prevent the formation of fibrous or gliotic tissue around the CRM or neuroimplant. These carriers are particularly useful for the practice of this embodiment, either alone, or in combination with a fibrosis (or gliosis) inhibiting composition. The following polymeric carriers can be infiltrated (as described in the previous paragraph) into the vicinity of the electrode-tissue interface and include: (a) sprayable collagen-containing formulations such as COSTASIS and CT3, either alone, or loaded with a fibrosis-inhibiting (or gliosis-inhibiting) agent, applied to the implantation site (or the implant/device surface); (b) sprayable PEG-containing formulations such as COSEAL, FOCALSEAL, SPRAYGEL or DURASEAL, either alone, or loaded with a fibrosis-inhibiting (or gliosis-inhibiting) agent, applied to the implantation site (or the implant/device surface); (c) fibrinogen-containing formulations such as FLOSEAL or TISSEAL, either alone, or loaded with a fibrosis-inhibiting (or gliosis-inhibiting) agent, applied to the implantation site (or the implant/device surface); (d) hyaluronic acid-containing formulations such as RESTYLANE, HYLAFORM, PERLANE, SYNVISC, SEPRAFILM, SEPRACOAT, InterGel, LUBRICOAT, loaded with a fibrosis-inhibiting (or gliosis-inhibiting) agent applied to the implantation site (or the implant/device surface); (e) polymeric gels for surgical implantation such as REPEL or FLOWGEL loaded with a fibrosis-inhibiting (or gliosis-inhibiting) agent applied to the implantation site (or the implant/device surface); (f) orthopedic “cements” used to hold prostheses and tissues in place loaded with a fibrosis-inhibiting (or gliosis-inhibiting) agent applied to the implantation site (or the implant/device surface), such as OSTEOBOND (Zimmer), low viscosity cement (LVC) (Wright Medical Technology), SIMPLEX P (Stryker), PALACOS (Smith & Nephew), and ENDURANCE (Johnson & Johnson, Inc.); (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT, either alone, or loaded with a fibrosis-inhibiting (or gliosis-inhibiting) agent, applied to the implantation site (or the implant/device surface); (h) implants containing hydroxyapatite [or synthetic bone material such as calcium sulfate, VITOSS (Orthovita) and CORTOSS (Orthovita)] loaded with a fibrosis-inhibiting (or gliosis-inhibiting) agent applied to the implantation site (or the implant/device surface); (i) other biocompatible tissue fillers loaded with a fibrosis-inhibiting (or gliosis-inhibiting) agent, such as those made by BioCure, 3M Company and Neomend, applied to the implantation site (or the implant/device surface); (j) polysaccharide gels such as the ADCON series of gels either alone, or loaded with a fibrosis-inhibiting (or gliosis-inhibiting) agent, applied to the implantation site (or the implant/device surface); and/or (k) films, sponges or meshes such as INTERCEED, VICRYL mesh, and GELFOAM loaded with a fibrosis-inhibiting (or gliosis-inhibiting) agent applied to the implantation site (or the implant/device surface).

An exemplary polymeric matrix which can be used to help prevent the formation of fibrous or gliotic tissue around the CRM or neuroimplant, either alone or in combination with a fibrosis (or gliosis) inhibiting agent/composition, may be formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another exemplary composition may comprise either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous or gliotic tissue around the CRM or neuroimplant.

Other examples of compositions that may be infiltrated into tissue adjacent to a medical device include compositions formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another exemplary composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix.

According to one aspect, any fibrosis-inhibiting and/or anti-infective agent or a composition comprising a fibrosis-inhibiting and/or anti-infective agent or a composition described herein may be utilized in the practice of the present invention. In one aspect of the invention, the subject compositions infiltrated into tissue adjacent to neurostimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

Additional examples of anti-scarring agents which can be used include those having a high potency in the assays described herein (approximately 1-100 nM IC₅₀ range) such as isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, and tacrolimus. For high potency drugs, the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁴ M of agent should be maintained on the implant or barrier surface.

Other examples of agents which can be used include those having a mid-potency in the assays described herein (approximately 100-500 nM IC₅₀ range) such as loteprednol etabonate, juglone, prednisolone, puromycin, 3-BAABE, cladribine, and mannose-6-phosphate. For mid-potency drugs, the total dose typically should not to exceed 500 mg (range of 1.0 μg to 500 mg) and preferably 1 μg to 200 mg; dose per unit area of 0.01 μg-200 μg per mm², preferably 0.1 μg/mm²-40 μg/mm²; and minimum concentration of 10⁻⁸-10⁻⁴ M of agent should be maintained on the implant or barrier surface.

Other examples of agents which can be used include those having a low potency in the assays described herein (approximately 500-1000 nm range IC₅₀ range) such as 5-azacytidine, Ly333531 (ruboxistaurin), and simvastatin. For low potency drugs, the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm²; and minimum concentration of 10⁻⁸-10⁻⁴ M of agent should to be maintained on the implant or barrier surface.

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

For greater clarity, several specific neurostimulation devices and treatments will be described in greater detail below.

(1) Neurostimulation for the Treatment of Chronic Pain

In one aspect, the subject agents or compositions may be infiltrated into tissue adjacent to a neurostimulation device for the management of chronic pain. The subject compositions may contain a therapeutic agent (e.g., an anti-scarring and/or anti-infective agent).

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. It has been reported that approximately 40 million Americans are afflicted with recurrent headaches and that the cost of medications for this condition exceeds $4 billion a year. A further 8 million people in the U.S. report that they experience chronic neck or facial pain and spend an estimated $2 billion a year for treatment. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, neurostimulation devices have been developed to treat severe intractable pain that is resistant to other traditional treatment modalities such as drug therapy, invasive therapy (surgery), or behavioral/lifestyle changes.

In principle, neurostimulation works by delivering low voltage electrical stimulation to the spinal cord or a particular peripheral nerve in order to block the sensation of pain. The Gate Control Theory of Pain (Ronald Melzack and Patrick Wall) hypothesizes that there is a “gate” in the dorsal horn of the spinal cord that controls the flow of pain signals from the peripheral receptors to the brain. It is speculated that the body can inhibit the pain signals (“close the gate”) by activating other (non-pain) fibers in the region of the dorsal horn. Neurostimulation devices are implanted in the epidural space of the spinal cord to stimulate non-noxious nerve fibers in the dorsal horn and mask the sensation of pain. As a result the patient typically experiences a tingling sensation (known as paresthesia) instead of pain. With neurostimulation, the majority of patients will report improved pain relief (50% reduction), increased activity levels and a reduction in the use of narcotics.

Pain management neurostimulation systems consist of a power source that generates the electrical stimulation, leads (typically 1 or 2) that deliver electrical stimulation to the spinal cord or targeted peripheral nerve, and an electrical connection that connects the power source to the leads. Neurostimulation systems can be battery powered, radio-frequency powered, or a combination of both. In general, there are two types of neurostimulation devices: those that are surgically implanted and are completely internal (i.e., the battery and leads are implanted), and those with internal (leads and radio-frequency receiver) and external (power source and antenna) components. For internal, battery-powered neurostimulators, an implanted, non-rechargeable battery and the leads are all surgically implanted. The settings of the totally implanted neurostimulator may be controlled by the host by using an external magnet and the implant has a lifespan of two to four years. For radio-frequency powered neurostimulators, the radio-frequency is transmitted from an externally worn source to an implanted passive receiver. The radio-frequency system enables greater power resources and thus, multiple leads may be used.

There are numerous neurostimulation devices that can be used for spinal cord stimulation in the management of pain control, postural positioning and other disorders. Examples of specific neurostimulation devices include those composed of a sensor that detects the position of the spine and a stimulator that automatically emits a series of pulses which decrease in amplitude when back is in a supine position. See e.g., U.S. Pat. Nos. 5,031,618 and 5,342,409. The neurostimulator may be composed of electrodes and a control circuit which generates pulses and rest periods based on intervals corresponding to the body's activity and regeneration period as a treatment for pain. See e.g., U.S. Pat. No. 5,354,320. The neurostimulator, which may be implanted within the epidural space parallel to the axis of the spinal cord, may transmit data to a receiver which generates a spinal cord stimulation pulse that may be delivered via a coupled, multi-electrode. See e.g., U.S. Pat. No. 6,609,031. The neurostimulator may be a stimulation catheter lead with a sheath and at least three electrodes that provide stimulation to neural tissue. See e.g., U.S. Pat. No. 6,510,347. The neurostimulator may be a self-centering epidual spinal cord lead with a pivoting region to stabilize the lead which inflates when injected with a hardening agent. See e.g., U.S. Pat. No. 6,308,103. Other neurostimulators used to induce electrical activity in the spinal cord are described in, e.g., U.S. Pat. Nos. 6,546,293; 6,236,892; 4,044,774 and 3,724,467.

Neurostimulation devices for the management of chronic pain, which may benefit from having the subject composition infiltrated into adjacent tissue according to the present invention, include commercially available products. Commercially available neurostimulation devices for the management of chronic pain include the SYNERGY, INTREL, X-TREL and MATTRIX neurostimulation systems from Medtronic, Inc. The percutaneous leads in this system can be quadripolar (4 electrodes), such as the PISCES-QUAD, PISCES-QUAD PLUS and the PISCES-QUAD Compact, or octapolar (8 electrodes) such as the OCTAD lead. The surgical leads themselves are quadripolar, such as the SPECIFY Lead, the RESUME II Lead, the RESUME TL Lead and the ON-POINT PNS Lead, to create multiple stimulation combinations and a broad area of paresthesia. These neurostimulation systems and associated leads may be described, for example, in U.S. Pat. Nos. 6,671,544; 6,654,642; 6,360,750; 6,353,762; 6,058,331; 5,342,409; 5,031,618 and 4,044,774. Neurostimulating leads such as these may benefit from release of a therapeutic agent able to reducing scarring at the electrode-tissue interface to increase the efficiency of impulse transmission and increase the duration that the leads function clinically. Nuerostimulating leads such as these may also benefit from release of a therapeutic agent able to prevent or inhibit infection in the vicinity of the implant site. In one aspect, the device includes neurostimulation devices for the management of chronic pain and/or leads having the subject composition comprising an anti-scarring agent and/or anti-infective agent infiltrated into tissue adjacent to where the device and/or leads are or will be implanted. In another aspect, the present invention provides leads having the subject composition comprising an anti-scarring agent and/or anti-infective agent infiltrated into tissue adjacent to the epidural space where the lead is or will be implanted. Other commercially available systems that may useful for the practice of this invention as described above include the rechargeable PRECISION Spinal Cord Stimulation System (Advanced Bionics Corporation, Sylmar, CA; which is a Boston Scientific Company) which can drive up to 16 electrodes (see e.g., U.S. Pat. Nos. 6,735,474; 6,735,475; 6,659,968; 6,622,048; 6,516,227 and 6,052,624). The GENESIS XP Spinal Cord Stimulator available from Advanced Neuromodulation Systems, Inc. (Plano, Tex.; see e.g., U.S. Pat. Nos. 6,748,276; 6,609,031 and 5,938,690) as well as the Vagus Nerve Stimulation (VNS) Therapy System available from Cyberonics, Inc. (Houston, Tex.; see e.g., U.S. Pat. Nos. 6,721,603 and 5,330,515) may also benefit from having the subject composition infiltrated into adjacent tissue according to the present invention.

Regardless of the specific design features, for neurostimulation to be effective in pain relief, the leads must be accurately positioned adjacent to the portion of the spinal cord or the targeted peripheral nerve that is to be electrically stimulated. Neurostimulators can migrate following surgery or excessive tissue growth or extracellular matrix deposition can occur around neurostimulators, which can lead to a reduction in the functioning of these devices. Neurostimulation devices having the subject compositions infiltrated into tissue adjacent to the electrode-tissue interface can be used to increase the duration that these devices clinically function. Neurostimulation devices may also benefit from release of a therapeutic agent able to prevent or inhibit infection in the vicinity of the implant site. In one aspect, the present invention provides neurostimulation devices for the management of chronic pain having the subject compositions infiltrated into tissue adjacent to the implanted portion (particularly the leads), where the subject compositions may include a therapeutic agent (e.g., an anti-scarring and/or anti-infective agent). Numerous polymeric and non-polymeric delivery systems for use in connection with neurostimulation devices for the management of chronic pain have been described above.

Therapeutic agents or pharmaceutical compositions may be infiltrated around implanted neurostimulation devices for the management of chronic pain by applying the composition directly and/or indirectly into and/or onto (a) tissue adjacent to the neurostimulation device for the management of chronic pain; (b) the vicinity of the neurostimulation device for the management of chronic pain-tissue interface; (c) the region around the neurostimulation device for the management of chronic pain; and (d) tissue surrounding the neurostimulation device for the management of chronic pain. Methods for infiltrating the subject compositions into tissue adjacent to a neurostimulation device for the management of chronic pain include delivering the composition: (a) to the surface of the neurostimulation device for the management of chronic pain (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the management of chronic pain; (c) to the surface of the neurostimulation device for the management of chronic pain and/or the tissue surrounding the implanted neurostimulation device for the management of chronic pain (e.g., as an injectable, paste, gel, in situ forming gel or mesh) immediately after the implantation of the neurostimulation device for the management of chronic pain; (d) by topical application of the composition into the anatomical space where the neurostimulation device for the management of chronic pain may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the management of chronic pain as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to neurostimulation devices for the management of chronic pain may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the management of chronic pain are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(2) Neurostimulation for the Treatment of Parkinson's Disease

In one aspect, the subject agents or compositions may be infiltrated into tissue adjacent to a neurostimulation device for the treatment of Parkinson's disease. The subject compositions may contain a therapeutic agent (e.g., an anti-scarring and/or anti-infective agent).

Neurostimulation devices implanted into the brain are used to control the symptoms associated with Parkinson's disease or essential tremor. Typically, these are dual chambered stimulator devices (similar to cardiac pacemakers) that deliver bilateral stimulation to parts of the brain that control motor function. Electrical stimulation is used to relieve muscular symptoms due to Parkinson's disease itself (tremor, rigidity, bradykinesia, akinesia) or symptoms that arise as a result of side effects of the medications used to treat the disease (dyskinesias). Two stimulating electrodes are implanted in the brain (usually bilaterally in the subthalamic nucleus or the globus pallidus interna) for the treatment of levodopa-responsive Parkinson's and one is implanted (in the ventral intermediate nucleus of the thalamus) for the treatment of tremor. The electrodes are implanted in the brain by a functional stereotactic neurosurgeon using a stereotactic head frame and MRI or CT guidance. The electrodes are connected via extensions (which run under the skin of the scalp and neck) to a neurostimulatory (pulse generating) device implanted under the skin near the clavicle. A neurologist can then optimize symptom control by adjusting stimulation parameters using a noninvasive control device that communicates with the neurostimulator via telemetry. The patient is also able to turn the system on and off using a magnet and control the device (within limits set by the neurologist) settings using a controller device. This form of deep brain stimulation has also been investigated for the treatment pain, epilepsy, psychiatric conditions (obsessive-compulsive disorder) and dystonia.

Several devices have been described for such applications including, for example, a neurostimulator and an implantable electrode that has a flexible, non-conducting covering material, which is used for tissue monitoring and stimulation of the cortical tissue of the brain as well as other tissue. See e.g., U.S. Pat. No. 6,024,702. The neurostimulator (pulse generator) may be an intracranially implanted electrical control module and a plurality of electrodes which stimulate the brain tissue with an electrical signal at a defined frequency. See e.g., U.S. Pat. No. 6,591,138. The neurostimulator may be a system composed of at least two electrodes adapted to the cranium and a control module adapted to be implanted beneath the scalp for transmitting output electrical signals and also external equipment for providing two-way communication. See e.g., U.S. Pat. No. 6,016,449. The neurostimulator may be an implantable assembly composed of a sensor and two electrodes, which are used to modify the electrical activity in the brain. See e.g., U.S. Pat. No. 6,466,822.

Neurostimulation devices for the treatment of Parkinson's disease, which may benefit from having the subject composition infiltrated into adjacent tissue according to the present invention, include commercially available products. A commercial example of a device used to treat Parkinson's disease and essential tremor includes the ACTIVA System by Medtronic, Inc. (see, for example, U.S. Pat. Nos., 6,671,544 and 6,654,642). This system consists of the KINETRA Dual Chamber neurostimulator, the SOLETRA neurostimulator or the INTREL neurostimulator, connected to an extension (an insulated wire), that is further connected to a DBS lead. The DBS lead consists of four thin, insulated, coiled wires bundled with polyurethane. Each of the four wires ends in a 1.5 mm long electrode. In one aspect, the present invention provides neurostimulation devices for the treatment of Parkinson's disease having the subject compositions infiltrated into tissue adjacent to where the device and/or leads are or will be implanted, where the subject compositions may include a therapeutic agent (e.g., an anti-scarring and/or anti-infective agent). In another aspect, the present invention provides leads (e.g., DBS leads) having the subject composition comprising an anti-scarring agent and/or anti-infective agent infiltrated into tissue adjacent to the tissue where the lead is or will be implanted. In another aspect, the present invention provides DBS leads having the subject composition comprising an anti-scarring agent and/or anti-infective agent infiltrated into the brain tissue adjacent to where the electrodes of the leads are or will be implanted.

Numerous polymeric and non-polymeric delivery systems for use in connection with neurostimulation devices for the treatment of Parkinson's disease have been described above.

Therapeutic agents or pharmaceutical compositions may be infiltrated around implanted neurostimulation devices for the treatment of Parkinson's disease by applying the composition directly and/or indirectly into and/or onto (a) tissue adjacent to the neurostimulation device for the treatment of Parkinson's disease; (b) the vicinity of the neurostimulation device for the treatment of Parkinson's disease-tissue interface; (c) the region around the neurostimulation device for the treatment of Parkinson's disease; and (d) tissue surrounding the neurostimulation device for the treatment of Parkinson's disease. Methods for infiltrating the subject compositions into tissue adjacent to a neurostimulation device for the treatment of Parkinson's disease include delivering the composition: (a) to the surface of the neurostimulation device for the treatment of Parkinson's disease (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of Parkinson's disease; (c) to the surface of the neurostimulation device for the treatment of Parkinson's disease and/or the tissue surrounding the implanted neurostimulation device for the treatment of Parkinson's disease (e.g., as an injectable, paste, gel, in situ forming gel or mesh) immediately after the implantation of the neurostimulation device for the treatment of Parkinson's disease; (d) by topical application of the composition into the anatomical space where the neurostimulation device for the treatment of Parkinson's disease may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of Parkinson's disease as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to neurostimulation devices for the treatment of Parkinson's disease may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of Parkinson's disease are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 4g-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(3) Vagal Nerve Stimulation for the Treatment of Epilepsy

In one aspect, the subject agents or compositions may be infiltrated into tissue adjacent to a neurostimulation device for the treatment of epilepsy. The subject compositions may contain a therapeutic agent (e.g., an anti-scarring and/or anti-infective agent).

Neurostimulation devices are also used for vagal nerve stimulation in the management of pharmacoresistant epilepsy (i.e., epilepsy that is uncontrolled despite appropriate medical treatment with ant-epileptic drugs). Approximately 30% of epileptic patients continue to have seizures despite of multiple attempts at controlling the disease with drug therapy or are unable to tolerate the side effects of their medications. It is estimated that approximately 2.5 million patients in the United States suffer from treatment-resistant epilepsy and may benefit from vagal nerve stimulation therapy. As such, inadequate seizure control remains a significant medical problem with many patients suffering from diminished self esteem, poor academic achievement and a restricted lifestyle as a result of their illness.

The vagus nerve (also called the 10^{th} cranial nerve) contains primarily afferent sensory fibres that carry information from the neck, thorax and abdomen to the nucleus tractus soltarius of the brainstem and on to multiple noradrenergic and serotonergic neuromodulatory systems in the brain and spinal cord. Vagal nerve stimulation (VNS) has been shown to induce progressive EEG changes, alter bilateral cerebral blood flow, and change blood flow to the thalamus. Although the exact mechanism of seizure control is not known, VNS has been demonstrated clinically to terminate seizures after seizure onset, reduce the severity and frequency of seizures, prevent seizures when used prophylactically over time, improve quality of life, and reduce the dosage, number and side effects of anti-epileptic medications (resulting in improved alertness, mood, memory).

In VNS, a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the neurologist to suit an individual patient's symptoms, while the patient can turn the device on and off through the use of an external magnet. Chronic electrical stimulation which can be used as a direct treatment for epilepsy is described in, for example, U.S. Pat. No. 6,016,449, whereby, an implantable neurostimulator is coupled to relatively permanent deep brain electrodes. The implantable neurostimulator may be composed of an implantable electrical lead having a furcated, or split, distal portion with two or more separate end segments, each of which bears at least one sensing or stimulation electrode, which may be used to treat epilepsy and other neurological disorders. See e.g., U.S. Pat. No. 6,597,953.

Neurostimulation devices for the treatment of epilepsy, which may benefit from having the subject composition infiltrated into adjacent tissue according to the present invention, include commercially available products. A commercial example of a VNS system is the product produced by Cyberonics, Inc. that includes the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,540,730 and 5,299,569.

Regardless of the specific design features, for vagal nerve stimulation to be effective in epilepsy, the leads must be accurately positioned adjacent to the left vagus nerve. If excessive scar tissue growth or extracellular matrix deposition occurs around the VNS leads, this can reduce the efficacy of the device. VNS devices having the subject compositions infiltrated into tissue adjacent can increase the efficiency of impulse transmission and increase the duration that these devices function clinically. VNS devices may also benefit from release of a therapeutic agent able to prevent or inhibit infection in the vicinity of the implant site. In one aspect, the device includes VNS devices and/or leads having the subject composition comprising an anti-scarring agent and/or anti-infective agent infiltrated into tissue adjacent to where the VNS device and/or leads are or will be implanted. In another aspect, the present invention provides leads having the subject composition comprising an anti-scarring agent and/or anti-infective agent infiltrated into tissue adjacent to the vagus nerve where the lead will be implanted.

In another aspect, the present invention provides neurostimulation devices for the treatment of epilepsy having the subject compositions infiltrated into adjacent tissue, where the subject compositions may include a therapeutic agent (e.g., an anti-scarring and/or anti-infective agent). Numerous polymeric and non-polymeric delivery systems for use in connection with neurostimulation devices for the treatment of epilepsy have been described above.

Therapeutic agents or pharmaceutical compositions may be infiltrated around implanted neurostimulation devices for the treatment of epilepsy by applying the composition directly and/or indirectly into and/or onto (a) tissue adjacent to the neurostimulation device for the treatment of epilepsy; (b) the vicinity of the neurostimulation device for the treatment of epilepsy-tissue interface; (c) the region around the neurostimulation device for the treatment of epilepsy; and (d) tissue surrounding the neurostimulation device for the treatment of epilepsy. Methods for infiltrating the subject compositions into tissue adjacent to a neurostimulation device for the treatment of epilepsy include delivering the composition: (a) to the surface of the neurostimulation device for the treatment of epilepsy (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of epilepsy; (c) to the surface of the neurostimulation device for the treatment of epilepsy and/or the tissue surrounding the implanted neurostimulation device for the treatment of epilepsy (e.g., as an injectable, paste, gel, in situ forming gel or mesh) immediately after the implantation of the neurostimulation device for the treatment of epilepsy; (d) by topical application of the composition into the anatomical space where the neurostimulation device for the treatment of epilepsy may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of epilepsy as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to neurostimulation devices for the treatment of epilepsy may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of epilepsy are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days, from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(4) Vagal Nerve Stimulation for the Treatment of Other Disorders

In one aspect, the subject agents or compositions may be infiltrated into tissue adjacent to a neurostimulation device for the treatment of neurological disorders. The subject compositions may contain a therapeutic agent (e.g., an anti-scarring and/or anti-infective agent).

It was discovered during the use of VNS for the treatment of epilepsy that some patients experienced an improvement in their mood during therapy. As such, VNS is currently being examined for use in the management of treatment-resistant mood disorders such as depression and anxiety. Depression remains an enormous clinical problem in the Western World with over 1% (25 million people in the United States) suffering from depression that is inadequately treated by pharmacotherapy. Vagal nerve stimulation has been examined in the management of conditions such as anxiety (panic disorder, obsessive-compulsive disorder, post-traumatic stress disorder), obesity, migraine, sleep disorders, dementia, Alzheimer's disease and other chronic or degenerative neurological disorders. VNS has also been examined for use in the treatment of medically significant obesity.

The implantable neurostimulator for the treatment of neurological disorders may be composed of an implantable electrical lead having a furcated, or split, distal portion with two or more separate end segments, each of which bears at least one sensing or stimulation electrode. See e.g., U.S. Pat. No. 6,597,953. The implantable neurostimulator may be an apparatus for treating Alzheimer's disease and dementia, particularly for neuro modulating or stimulating left vagus nerve, composed of an implantable lead-receiver, external stimulator, and primary coil. See e.g., U.S. Pat. No. 6,615,085.

Neurostimulation devices for the treatment of neurological disorders, which may benefit from having the subject composition infiltrated into adjacent tissue according to the present invention, include commercially available products. Cyberonics, Inc. manufactures the commercially available VNS system, including the Model 300 and Model 302 leads, the Model 101 and Model 102R pulse generators, the Model 201 programming wand and Model 250 programming software, and the Model 220 magnets. These products as well as others that are being developed by Cyberonics, Inc. may be used to treat neurological disorders, including depression (see e.g., U.S. Pat. No. 5,299,569), dementia (see e.g., U.S. Pat. No. 5,269,303), migraines (see e.g., U.S. Pat. No. 5,215,086), sleep disorders (see e.g., U.S. Pat. No. 5,335,657) and obesity (see e.g., U.S. Pat. Nos. 6,587,719; 6,609,025; 5,263,480 and 5,188,104).

It is important to note that the fundamentals of treatment are identical to those described above for epilepsy. The devices employed and the principles of therapy are also similar. As was described above for the treatment of epilepsy, if excessive scar tissue growth or extracellular matrix deposition occurs around the VNS leads, this can reduce the efficacy of the device. VNS devices may benefit from release of a therapeutic agent able to reducing scarring at the electrode-tissue interface to increase the efficiency of impulse transmission and increase the duration that these devices function clinically for the treatment of depression, anxiety, obesity, sleep disorders and dementia. VNS devices may also benefit from release of a therapeutic agent able to prevent or inhibit infection in the vicinity of the implant site. In one aspect, the device includes VNS devices and/or leads having the subject composition comprising an anti-scarring agent and/or anti-infective agent infiltrated into tissue adjacent to where the VNS device and/or leads are or will be implanted. In another aspect, the present invention provides leads having the subject composition comprising an anti-scarring agent and/or anti-infective agent infiltrated into tissue adjacent to the vagus nerve where the lead will be implanted.

In another aspect, the present invention provides neurostimulation devices for the treatment of neurological disorders having the subject compositions infiltrated into adjacent tissue, where the subject compositions may include a therapeutic agent (e.g., an anti-scarring and/or anti-infective agent). Numerous polymeric and non-polymeric delivery systems for use in connection with neurostimulation devices for the treatment of neurological disorders have been described above.

Therapeutic agents or pharmaceutical compositions may be infiltrated around implanted neurostimulation devices for the treatment of neurological disorders by applying the composition directly and/or indirectly into and/or onto (a) tissue adjacent to the neurostimulation device for the treatment of neurological disorders; (b) the vicinity of the neurostimulation device for the treatment of neurological disorders-tissue interface; (c) the region around the neurostimulation device for the treatment of neurological disorders; and (d) tissue surrounding the neurostimulation device for the treatment of neurological disorders. Methods for infiltrating the subject compositions into tissue adjacent to a neurostimulation device for the treatment of neurological disorders include delivering the composition: (a) to the surface of the neurostimulation device for the treatment of neurological disorders (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation device for the treatment of neurological disorders; (c) to the surface of the neurostimulation device for the treatment of neurological disorders and/or the tissue surrounding the implanted neurostimulation device for the treatment of neurological disorders (e.g., as an injectable, paste, gel, in situ forming gel or mesh) immediately after the implantation of the neurostimulation device for the treatment of neurological disorders; (d) by topical application of the composition into the anatomical space where the neurostimulation device for the treatment of neurological disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation device for the treatment of neurological disorders as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to neurostimulation devices for the treatment of neurological disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation devices for the treatment of neurological disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(5) Sacral Nerve Stimulation for Bladder Control Problems

In one aspect, the subject agents or compositions may be infiltrated into tissue adjacent to a neurostimulation system to treat bladder conditions. The subject compositions may contain a therapeutic agent (e.g., an anti-scarring and/or anti-infective agent).

Sacral nerve stimulation is used in the management of patients with urinary control problems such as urge incontinence, nonobstructive urinary retention, or urgency-frequency. Millions of people suffer from bladder control problems and a significant percentage (estimated to be in excess of 60%) is not adequately treated by other available therapies such as medications, absorbent pads, external collection devices, bladder augmentation or surgical correction. This can be a debilitating medical problem that can cause severe social anxiety and cause people to become isolated and depressed.

Mild electrical stimulation of the sacral nerve is used to influence the functioning of the bladder, urinary sphincter, and the pelvic floor muscles (all structures which receive nerve supply from the sacral nerve). An electrical lead is surgically implanted adjacent to the sacral nerve and a neurostimulator is implanted subcutaneously in the upper buttock or abdomen; the two are connected by an extension. The use of tined leads allows sutureless anchoring of the leads and minimally-invasive placement of the leads under local anesthesia. A handheld programmer is available for adjustment of the device by the attending physician and a patient-controlled programmer is available to adjust the settings and to turn the device on and off. The pulses are adjusted to provide bladder control and relieve the patient's symptoms.

Several neurostimulation systems have been described for sacral nerve stimulation in which electrical stimulation is targeted towards the bladder, pelvic floor muscles, bowel and/or sexual organs. For example, the neurostimulator may be an electrical stimulation system composed of an electrical stimulator and leads having insulator sheaths, which may be anchored in the sacrum using minimally-invasive surgery. See e.g., U.S. Pat. No. 5,957,965. In another aspect, the neurostimulator may be used to condition pelvic, sphincter or bladder muscle tissue. For example, the neurostimulator may be intramuscular electrical stimulator composed of a pulse generator and an elongated medical lead that is used for electrically stimulating or sensing electrical signals originating from muscle tissue. See e.g., U.S. Pat. No. 6,434,431. Another neurostimulation system consists of a leadless, tubular-shaped microstimulator that is implanted at pelvic floor muscles or associated nerve tissue that need to be stimulated to treat urinary incontinence. See e.g., U.S. Pat. No. 6,061,596.

Neurostimulation systems to treat bladder conditions, which may benefit from having the subject composition infiltrated into adjacent tissue according to the present invention, include commercially available products. A commercially available example of a neurostimulation system to treat bladder conditions is the INTERSTIM Sacral Nerve Stimulation System made by Medtronic, Inc. See e.g., U.S. Pat. Nos. 6,104,960; 6,055,456 and 5,957,965.

Regardless of the specific design features, for bladder control therapy to be effective, the leads must be accurately positioned adjacent to the sacral nerve, bladder, sphincter or pelvic muscle (depending upon the particular system employed). If excessive scar tissue growth or extracellular matrix deposition occurs around the leads, efficacy can be compromised. Sacral nerve stimulating devices (such as INTERSTIM) having the subject compositions infiltrated into tissue adjacent to the electrode-tissue interface can increase the efficiency of impulse transmission and increase the duration that these devices function clinically. Nuerostimulating devices such as these may also benefit from release of a therapeutic agent able to prevent or inhibit infection in the vicinity of the implant site. In one aspect, the device includes sacral nerve stimulating devices and/or leads having the subject composition comprising an anti-scarring agent and/or anti-infective agent infiltrated into tissue adjacent to where the sacral nerve stimulating device and/or leads are or will be implanted. In another aspect, the present invention provides leads having the subject composition comprising an anti-scarring agent and/or anti-infective agent infiltrated into tissue adjacent to the sacral nerve where the lead will be implanted.

For devices designed to stimulate the bladder or pelvic muscle tissue directly, slightly different embodiments may be required. In this aspect, the device includes bladder or pelvic muscle stimulating devices, leads, and/or sensors having the subject composition comprising an anti-scarring agent and/or anti-infective agent infiltrated into tissue adjacent to where the sacral nerve stimulating device and/or leads are or will be implanted. In another aspect, the present invention provides leads and/or sensors, which are delivering an impulse or monitoring the activity of the muscle, having the subject composition comprising an anti-scarring agent and/or anti-infective agent infiltrated into tissue adjacent to the tissue (e.g., muscle) where the lead and/or sensor will be implanted.

In another aspect, the present invention provides neurostimulation systems to treat bladder conditions having the subject compositions infiltrated into adjacent tissue, where the subject compositions may include a therapeutic agent (e.g., an anti-scarring and/or anti-infective agent). Numerous polymeric and non-polymeric delivery systems for use in connection with neurostimulation systems to treat bladder conditions have been described above.

Therapeutic agents or pharmaceutical compositions may be infiltrated around implanted neurostimulation systems to treat bladder conditions by applying the composition directly and/or indirectly into and/or onto (a) tissue adjacent to the neurostimulation system to treat bladder conditions; (b) the vicinity of the neurostimulation system to treat bladder conditions-tissue interface; (c) the region around the neurostimulation system to treat bladder conditions; and (d) tissue surrounding the neurostimulation system to treat bladder conditions. Methods for infiltrating the subject compositions into tissue adjacent to a neurostimulation system to treat bladder conditions include delivering the composition: (a) to the surface of the neurostimulation system to treat bladder conditions (e.g., as an injectable, paste, gel or mesh) during the implantation procedure; (b) to the surface of the tissue (e.g., as an injectable, paste, gel, in situ forming gel or mesh) immediately prior to, or during, implantation of the neurostimulation system to treat bladder conditions; (c) to the surface of the neurostimulation system to treat bladder conditions and/or the tissue surrounding the implanted neurostimulation system to treat bladder conditions (e.g., as an injectable, paste, gel, in situ forming gel or mesh) immediately after the implantation of the neurostimulation system to treat bladder conditions; (d) by topical application of the composition into the anatomical space where the neurostimulation system to treat bladder conditions may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the neurostimulation system to treat bladder conditions as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to neurostimulation systems to treat bladder conditions may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As neurostimulation systems to treat bladder conditions are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(6) Gastric Nerve Stimulation for the Treatment of GI Disorders

In one aspect, the subject agents or compositions may be infiltrated into tissue adjacent to a device for treatment of GI disorders. The subject compositions may contain a therapeutic agent (e.g., an anti-scarring and/or anti-infective agent).

Neurostimulator of the gastric nerve (which supplies the stomach and other portions of the upper GI tract) is used to influence gastric emptying and satiety sensation in the management of clinically significant obesity or problems associated with impaired GI motility. Morbid obesity has reached epidemic proportions and is thought to affect over 25 million Americans and lead to significant health problems such as diabetes, heart attack, stroke and death. Mild electrical stimulation of the gastric nerve is used to influence the functioning of the upper GI tract and stomach (all structures which receive nerve supply from the gastric nerve). An electrical lead is surgically implanted adjacent to the gastric nerve and a neurostimulator is implanted subcutaneously; the two are connected by an extension. A handheld programmer is available for adjustment of the device by the attending physician and a patient-controlled programmer is available to adjust the settings and to turn the device on and off. The pulses are adjusted to provide a sensation of satiety and relieve the sensation of hunger experienced by the patient. This can reduce the amount of food (and hence caloric) intake and allow the patient to lose weight successfully. Related devices include neurostimulation devices used to stimulate gastric emptying in patients with impaired gastric motility, a neurostimulator to promote bowel evacuation in patients with constipation (stimulation is delivered to the colon), and devices targeted at the bowel for patients with other GI motility disorders.

Several such devices have been described including, for example, a sensor that senses electrical activity in the gastrointestinal tract which is coupled to a pulse generator that emits and inhibits asynchronous stimulation pulse trains based on the natural gastrointestinal electrical activity. See e.g., U.S. Pat. No. 5,995,872. Other neurostimulation devices deliver impulses to the colon and rectum to manage constipation and are composed of electrical leads, electrodes and an implanted stimulation generator. See e.g., U.S. Pat. No. 6,026,326. The neurostimulator may be a pulse generator and electrodes that electrically stimulate the neuromuscular tissue of the viscera to treat obesity. See e.g., U.S. Pat. No. 6,606,523. The neurostimulator may be a hermetically sealed implantable pulse generator that is electrically coupled to the gastrointestinal tract and emits two rates of electrical stimulation to treat gastroparesis for patients with impaired gastric emptying. See e.g., U.S. Pat. No. 6,091,992. The neurostimulator may be composed of an electrical signal controller, connector wire and attachment lead which generates continuous low voltage electrical stimulation to the fundus of the stomach to control appetite. See e.g., U.S. Pat. No. 6,564,101. Other neurostimulators that are used to electrically stimulate the gastrointestinal tract are described in, e.g., U.S. Pat. Nos. 6,453,199; 6,449,511 and 6,243,607.

Devices for treatment of GI disorders, which may benefit from having the subject composition infiltrated into adjacent tissue according to the present invention, include commercially available products. A commercially available example of a gastric nerve stimulation device for use with the present invention is the TRANSCEND Implantable Gastric Stimulator (IGS), which is currently being developed by Transneuronix, Inc. (Mt. Arlington, N.J.). The IGS is a programmable, bipolar pulse generator that delivers small bursts of electrical pulses through the lead to the stomach wall to treat obesity. See, e.g., U.S. Pat. Nos. 6,684,104 and 6,165,084.

Regardless of the specific design features, for gastric nerve stimulation to be effective in satiety control (or gastroparesis), the leads must be accurately positioned adjacent to the gastric nerve. If excessive scar tissue growth or extracellular matrix deposition occurs around the leads, efficacy can be compromised. Gastric nerve stimulating devices (and other implanted devices designed to influence GI motility) having the subject compositions infiltrated into tissue adjacent to the electrode-tissue interface can increase the efficiency of impulse transmission and increase the duration that these devices function clinically. Gastric nerve stimulating devices (and other implanted devices designed to influence GI motility) may also benefit from release of a therapeutic agent able to prevent or inhibit infection in the vicinity of the implant site. In one aspect, the device includes gastric nerve stimulating devices and/or leads having the subject composition comprising an anti-scarring agent and/or anti-infective agent infiltrated into tissue adjacent to where the gastric nerve stimulating device and/or leads are or will be implanted. In another aspect, the present invention provides leads having the subject composition comprising an anti-scarring agent and/or anti-infective agent infiltrated into tissue adjacent to the gastric nerve where the lead will be implanted.

In another aspect, the present invention provides devices for treatment of GI disorders having the subject compositions infiltrated into adjacent tissue, where the subject compositions may include a therapeutic agent (e.g., an anti-scarring and/or anti-infective agent). Numerous polymeric and non-polymeric delivery systems for use in connection with devices for treatment of GI disorders have been described above.

Therapeutic agents or pharmaceutical compositions may be infiltrated around implanted devices for treatment of GI disorders by applying the composition directly and/or indirectly into and/or onto (a) tissue adjacent to the device for treatment of GI disorders; (b) the vicinity of the device for treatment of GI disorders-tissue interface; (c) the region around the device for treatment of GI disorders; and (d) tissue surrounding the device for treatment of GI disorders. Methods for infiltrating the subject compositions into tissue adjacent to a device for treatment of GI disorders include delivering the composition: (a) to the surface of the device for treatment of GI disorders (e.g., as an injectable, paste, gel or mesh) during the implantation procedure; (b) to the surface of the tissue (e.g., as an injectable, paste, gel, in situ forming gel or mesh) immediately prior to, or during, implantation of the device for treatment of GI disorders; (c) to the surface of the device for treatment of GI disorders and/or the tissue surrounding the implanted device for treatment of GI disorders (e.g., as an injectable, paste, gel, in situ forming gel or mesh) immediately after the implantation of the device for treatment of GI disorders; (d) by topical application of the composition into the anatomical space where the device for treatment of GI disorders may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the device for treatment of GI disorders as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to devices for treatment of GI disorders may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As devices for treatment of GI disorders are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 11g-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(7) Cochlear Implants for the Treatment of Deafness

In one aspect, the subject agents or compositions may be infiltrated into tissue adjacent to a cochlear implant. The subject compositions may contain a therapeutic agent (e.g., an anti-scarring and/or anti-infective agent).

Neurostimulation is also used in the form of a cochlear implant that stimulates the auditory nerve for correcting sensorineural deafness. A sound processor captures sound from the environment and processes it into a digital signal that is transmitted via an antenna through the skin to the cochlear implant. The cochlear implant, which is surgically implanted in the cochlea adjacent to the auditory nerve, converts the digital information into electrical signals that are communicated to the auditory nerve via an electrode array. Effectively, the cochlear implant serves to bypass the nonfunctional cochlear transducers and directly depolarize afferent auditory nerve fibers. This stimulates the nerve to send signals to the auditory center in the brain and allows the patient to “hear” the sounds detected by the sound processor. The treatment is used for adults with 70 dB or greater hearing loss (and able to understand up to 50% of words in a sentence using a hearing aid) or children 12 months or older with 90 dB hearing loss in both ears.

Although many implantations are performed without incident, approximately 12-15% of patients experience some complications. Histologic assessment of cochlear implants has revealed that several forms of injury and scarring can occur. Surgical trauma can induce cochlear fibrosis, cochlear neossification and injury to the membranous cochlea (including loss of the sensorineural elements). A foreign body reaction along the implant and the electrode can produce a fibrous tissue response along the electrode array that has been associated with implant failure. Implantation of a neurostimulation device may also introduce or promote infection in the vicinity of the implant site.

A variety of suitable cochlear implant systems or “bionic ears” have been described for use in association with this invention. For example, the neurostimulator may be composed of a plurality of transducer elements which detect vibrations and then generates a stimulus signal to a corresponding neuron connected to the cranial nerve. See e.g., U.S. Pat. No. 5,061,282. The neurostimulator may be a cochlear implant having a sound-to-electrical stimulation encoder, a body implantable receiver-stimulator and electrodes, which emit pulses based on received electrical signals. See e.g., U.S. Pat. No. 4,532,930. The neurostimulator may be an intra-cochlear apparatus that is composed of a transducer that converts an audio signal into an electrical signal and an electrode array which electrically stimulates predetermined locations of the auditory nerve. See e.g., U.S. Pat. No. 4,400,590. The neurostimulator may be a stimulus generator for applying electrical stimuli to any branch of the 8^{th} nerve in a generally constant rate independent of audio modulation, such that it is perceived as active silence. See e.g., U.S. Pat. No. 6,175,767. The neurostimulator may be a subcranially implanted electromechanical system that has an input transducer and an output stimulator that converts a mechanical sound vibration into an electrical signal. See e.g., U.S. Pat. No. 6,235,056. The neurostimulator may be a cochlear implant that has a rechargeable battery housed within the implant for storing and providing electrical power. See e.g., U.S. Pat. No. 6,067,474. Other neurostimulators that are used as cochlear implants are described in, e.g., U.S. Pat. Nos. 6,358,281; 6,308,101 and 5,603,726.

Cochlear implants, which may benefit from having the subject composition infiltrated into adjacent tissue according to the present invention, include commercially available products. Several commercially available devices are available for the treatment of patients with significant sensorineural hearing loss and are suitable for use with the present invention. For example, the HIRESOLUTION Bionic Ear System (Boston Scientific Corp., Nattick, Mass.) consists of the HIRES AURIA Processor which processes sound and sends a digital signal to the HIRES 90K Implant that has been surgically implanted in the inner ear. See e.g., U.S. Pat. Nos. 6,636,768; 6,309,410 and 6,259,951. The electrode array that transmits the impulses generated by the HIRES 90K Implant to the nerve may benefit from having the subject composition infiltrated into tissue adjacent to the electrode-nerve interface. The PULSARci cochlear implant (MED-EL GMBH, Innsbruck, Austria, see e.g., U.S. Pat. Nos. 6,556,870 and 6,231,604) and the NUCLEUS 3 cochlear implant system (Cochlear Corp., Lane Cove, Australia, see e.g., U.S. Pat. Nos. 6,807,445; 6,788,790; 6,554,762; 6,537,200 and 6,394,947) are other commercial examples of cochlear implants whose electrodes may benefit from having the subject composition infiltrated into tissue adjacent to the electrode-nerve interface.

Regardless of the specific design features, for cochlear implants to be effective in sensorineural deafness, the electrode arrays must be accurately positioned adjacent to the afferent auditory nerve fibers. If excessive scar tissue growth or extracellular matrix deposition occurs around the leads, efficacy can be compromised. Cochlear implants having the subject compositions infiltrated into tissue adjacent to the electrode-tissue interface can increase the efficiency of impulse transmission and increase the duration that these devices function clinically. Cochlear implants may also benefit from release of a therapeutic agent able to prevent or inhibit infection in the vicinity of the implant site. In one aspect, the device includes cochlear implants and/or leads having the subject composition comprising an anti-scarring agent and/or anti-infective agent infiltrated into tissue adjacent to where the cochlear implant and/or leads are or will be implanted. In another aspect, the present invention provides leads having the subject composition comprising an anti-scarring agent and/or anti-infective agent infiltrated into tissue adjacent to the cochlear tissue surrounding the lead.

In another aspect, the present invention provides cochlear implants having the subject compositions infiltrated into adjacent tissue, where the subject compositions may include a therapeutic agent (e.g., an anti-scarring and/or anti-infective agent). Numerous polymeric and non-polymeric delivery systems for use in connection with cochlear implants have been described above.

Therapeutic agents or pharmaceutical compositions may be infiltrated around implanted cochlear implants by applying the composition directly and/or indirectly into and/or onto (a) tissue adjacent to the cochlear implant; (b) the vicinity of the cochlear implant-tissue interface; (c) the region around the cochlear implant; and (d) tissue surrounding the cochlear implant. Methods for infiltrating the subject compositions into tissue adjacent to a cochlear implant include delivering the composition: (a) to the surface of the cochlear implant (e.g., as an injectable, paste, gel or mesh) during the implantation procedure; (b) to the surface of the tissue (e.g., as an injectable, paste, gel, in situ forming gel or mesh) immediately prior to, or during, implantation of the cochlear implant; (c) to the surface of the cochlear implant and/or the tissue surrounding the implanted cochlear implant (e.g., as an injectable, paste, gel, in situ forming gel or mesh) immediately after the implantation of the cochlear implant; (d) by topical application of the composition into the anatomical space where the cochlear implant may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cochlear implant as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to cochlear implants may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cochlear implants are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days Generally, the release time may also be from about less than 1 day to about 180 days; from about 7 days to about 14 days; from about 14 days to about 28 days, from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(8) Electrical Stimulation to Promote Bone Growth

In one aspect, the subject agents or compositions may be infiltrated into tissue adjacent to an electrical bone stimulation device. The subject compositions may contain a therapeutic agent (e.g., an anti-scarring and/or anti-infective agent).

Electrical stimulation can also be used to stimulate bone growth. For example, the stimulation device may be an electrode and generator having a strain response piezoelectric material which responds to strain by generating a charge to enhance the anchoring of an implanted bone prosthesis to the natural bone. See e.g., U.S. Pat. No. 6,143,035. If excessive scar tissue growth or extracellular matrix deposition occurs around the leads, efficacy can be compromised. Electrical bone stimulation devices having the subject compositions infiltrated into tissue adjacent to the electrode-tissue interface can increase the efficiency of impulse transmission and increase the duration that these devices function clinically. Electrical bone stimulation devices may also benefit from release of a therapeutic agent able to prevent or inhibit infection in the vicinity of the implant site. In one aspect, the device includes electrical bone stimulation devices and/or leads having the subject composition comprising an anti-scarring agent and/or anti-infective agent infiltrated into tissue adjacent to where the electrical bone stimulation device and/or leads are or will be implanted. In another aspect, the present invention provides leads having the subject composition comprising an anti-scarring agent and/or anti-infective agent infiltrated into tissue adjacent to the bone tissue surrounding the electrical lead.

In another aspect, the present invention provides electrical bone stimulation devices having the subject compositions infiltrated into adjacent tissue, where the subject compositions may include a therapeutic agent (e.g., an anti-scarring and/or anti-infective agent). Numerous polymeric and non-polymeric delivery systems for use in connection with electrical bone stimulation devices have been described above.

Therapeutic agents or pharmaceutical compositions may be infiltrated around implanted electrical bone stimulation devices by applying the composition directly and/or indirectly into and/or onto (a) tissue adjacent to the electrical bone stimulation device; (b) the vicinity of the electrical bone stimulation device-tissue interface; (c) the region around the electrical bone stimulation device; and (d) tissue surrounding the electrical bone stimulation device. Methods for infiltrating the subject compositions into tissue adjacent to an electrical bone stimulation device include delivering the composition: (a) to the surface of the electrical bone stimulation device (e.g., as an injectable, paste, gel or mesh) during the implantation procedure; (b) to the surface of the tissue (e.g., as an injectable, paste, gel, in situ forming gel or mesh) immediately prior to, or during, implantation of the electrical bone stimulation device; (c) to the surface of the electrical bone stimulation device and/or the tissue surrounding the implanted electrical bone stimulation device (e.g., as an injectable, paste, gel, in situ forming gel or mesh) immediately after the implantation of the electrical bone stimulation device; (d) by topical application of the composition into the anatomical space where the electrical bone stimulation device may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the electrical bone stimulation device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to electrical bone stimulation devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As electrical bone stimulation devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Although numerous neurostimulation devices have been described above, all possess similar design features and cause similar unwanted tissue reactions following implantation and may introduce or promote infection in the area of the implant site. It should be obvious to one of skill in the art that commercial neurostimulation devices not specifically sited above as well as next-generation and/or subsequently-developed commercial neurostimulation products are to be anticipated and are suitable for use under the present invention. The neurostimulation device, particularly the lead(s), must be positioned in a very precise manner to ensure that stimulation is delivered to the correct anatomical location in the nervous system. All, or parts, of a neurostimulation device can migrate following surgery, or excessive scar (or glial) tissue growth can occur around the implant, which can lead to a reduction in the performance of these devices. Neurostimulator devices having the subject compositions infiltrated into tissue adjacent to the electrode-tissue interface can be used to increase the efficacy and/or the duration of activity of the implant (particularly for fully-implanted, battery-powered devices). Neurostimulator devices may also benefit from release of a therapeutic agent able to prevent or inhibit infection in the vicinity of the implant site. In one aspect, the present invention provides neurostimulator devices having the subject compositions infiltrated into adjacent tissue, where the subject compositions may include a therapeutic agent (e.g., an anti-scarring and/or anti-infective agent). Numerous polymeric and non-polymeric delivery systems for use in connection with neurostimulator devices have been described above. These compositions can further include one or more fibrosis-inhibiting agents such that the overgrowth of granulation, fibrous, or gliotic tissue is inhibited or reduced and/or one or more anti-infective agents such that infection in the vicinity of the implant site is inhibited or prevented.

Cardiac Rhythm Management (CRM) Devices

In one aspect, the subject agents or compositions may be infiltrated into tissue adjacent to a cardiac rhythm management device. The subject compositions may contain a therapeutic agent (e.g., an anti-scarring and/or anti-infective agent).

The medical device may also be a cardiac pacemaker device where a pulse generator delivers an electrical impulse to myocardial tissue (often specialized conduction fibres) via an implanted lead in order to regulate cardiac rhythm. Typically, electrical leads are composed of a connector assembly, a lead body (i.e., conductor) and an electrode. Electrical leads may be unipolar, in which they are adapted to provide effective therapy with only one electrode. Multi-polar leads are also available, including bipolar, tripolar and quadripolar leads. Electrical leads may also have insulating sheaths which may include polyurethane or silicone-rubber coatings. Representative examples of electrical leads include, without limitation, medical leads, cardiac leads, pacer leads, pacing leads, pacemaker leads, endocardial leads, endocardial pacing leads, cardioversion/defibrillator leads, cardioversion leads, epicardial leads, epicardial defibrillator leads, patch defibrillators, patch leads, electrical patch, transvenous leads, active fixation leads, passive fixation leads and sensing leads Representative examples of CRM devices that utilize electrical leads include: pacemakers, LVAD's, defibrillators, implantable sensors and other electrical cardiac stimulation devices.

There are numerous pacemaker devices where the occurrence of a fibrotic reaction will adversely affect the functioning of the device or cause damage to the myocardial tissue. Typically, fibrotic encapsulation of the pacemaker lead (or the growth of fibrous tissue between the lead and the target myocardial tissue) slows, impairs, or interrupts electrical transmission of the impulse from the device to the myocardium. For example, fibrosis is often found at the electrode-myocardial interfaces in the heart, which may be attributed to electrical injury from focal points on the electrical lead. The fibrotic injury may extend into the tricuspid valve, which may lead to perforation. Fibrosis may lead to thrombosis of the subclavian vein; a condition which may be life-threatening. Electrical leads having the subject compositions infiltrated into tissue adjacent to the electrode-tissue interface may help prolong the clinical performance of these devices. Not only can fibrosis cause the device to function suboptimally or not at all, it can cause excessive drain on battery life as increased energy is required to overcome the electrical resistance imposed by the intervening scar tissue. Similarly, fibrotic encapsulation of the sensing components of a rate-responsive pacemaker (described below) can impair the ability of the pacemaker to identify and correct rhythm abnormalities leading to inappropriate pacing of the heart or the failure to function correctly when required. Cardiac pacemaker devices and/or electrical leads may also benefit from release of a therapeutic agent able to prevent or inhibit infection in the vicinity of the implant site.

Several different electrical pacing devices are used in the treatment of various cardiac rhythm abnormalities including pacemakers, implantable cardioverter defibrillators (ICD), left ventricular assist devices (LVAD), and vagus nerve stimulators (stimulates the fibers of the vagus nerve which in turn innervate the heart). The pulse generating portion of device sends electrical impulses via implanted leads to the muscle (myocardium) or conduction tissue of the heart to affect cardiac rhythm or contraction. Pacing can be directed to one or more chambers of the heart. Cardiac pacemakers may be used to block, mask, or stimulate electrical signals in the heart to treat dysfunctions, including, without limitation, atrial rhythm abnormalities, conduction abnormalities and ventricular rhythm abnormalities. ICDs are used to depolarize the ventricals and re-establish rhythm if a ventricular arrhythmia occurs (such as asystole or ventricular tachycardia) and LVADs are used to assist ventricular contraction in a failing heart.

Representative examples of patents which describe pacemakers and pacemaker leads include U.S. Pat. Nos. 4,662,382, 4,782,836, 4,856,521, 4,860,751, 5,101,824, 5,261,419, 5,284,491, 6,055,454, 6,370,434, and 6,370,434. Representative examples of electrical leads include those found on a variety of cardiac devices, such as cardiac stimulators (see e.g., U.S. Pat. Nos. 6,584,351 and 6,115,633), pacemakers (see e.g., U.S. Pat. Nos. 6,564,099; 6,246,909 and 5,876,423), implantable cardioverter-defibrillators (ICDs), other defibrillator devices (see e.g., U.S. Pat. No. 6,327,499), defibrillator or demand pacer catheters (see e.g., U.S. Pat. No. 5,476,502) and Left Ventricular Assist Devices (see e.g., U.S. Pat. No. 5,503,615).

Cardiac rhythm devices, and in particular the lead(s) that deliver the electrical pulsation, must be positioned in a very precise manner to ensure that stimulation is delivered to the correct anatomical location in the heart. All, or parts, of a pacing device can migrate following surgery, or excessive scar tissue growth can occur around the lead, which can lead to a reduction in the performance of these devices (as described previously). Cardiac rhythm management devices having the subject compositions infiltrated into tissue adjacent to the electrode-tissue interface can be used to increase the efficacy and/or the duration of activity (particularly for fully-implanted, battery-powered devices) of the implant. Cardiac rhythm management devices may also benefit from release of a therapeutic agent able to prevent or inhibit infection in the vicinity of the implant site. In one aspect, the present invention provides cardiac rhythm management devices and/or leads having the subject composition comprising an anti-scarring agent and/or anti-infective agent infiltrated into tissue adjacent to where the cardiac rhythm management device and/or leads are or will be implanted. In another aspect, the present invention provides leads having the subject composition comprising an anti-scarring agent and/or anti-infective agent infiltrated into tissue adjacent to the tissue where the lead will be implanted.

In another aspect, the present invention provides cardiac rhythm management devices having the subject compositions infiltrated into adjacent tissue, where the subject compositions may include a therapeutic agent (e.g., an anti-scarring and/or anti-infective agent). Numerous polymeric and non-polymeric delivery systems for use in connection with cardiac rhythm management devices have been described above.

Therapeutic agents or pharmaceutical compositions may be infiltrated around implanted cardiac rhythm management devices by applying the composition directly and/or indirectly into and/or onto (a) tissue adjacent to the cardiac rhythm management device; (b) the vicinity of the cardiac rhythm management device-tissue interface; (c) the region around the cardiac rhythm management device; and (d) tissue surrounding the cardiac rhythm management device. Methods for infiltrating the subject compositions into tissue adjacent to a cardiac rhythm management device include delivering the composition: (a) to the surface of the cardiac rhythm management device (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac rhythm management device; (c) to the surface of the cardiac rhythm management device and/or the tissue surrounding the implanted cardiac rhythm management device (e.g., as an injectable, paste, gel, in situ forming gel or mesh) immediately after the implantation of the cardiac rhythm management device; (d) by topical application of the composition into the anatomical space where the cardiac rhythm management device may be placed (particularly useful for this embodiment is the use of polymeric carriers which release the therapeutic agent over a period ranging from several hours to several weeks-fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac rhythm management device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to cardiac rhythm management devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cardiac rhythm management devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

Additional examples of anti-scarring agents which can be used include those having a high potency in the assays described herein (approximately 1-100 nM IC₅₀ range) such as isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, and tacrolimus. For high potency drugs, the total dose typically should not exceed 200 mg (range of 0.1 μg to 200 mg) and preferably 1 μg to 100 mg; dose per unit area of 0.01 μg-100 μg per mm²; preferably 0.1 μg/mm²-20 μg/mm²; and minimum concentration of 10⁻⁸-10⁻⁴ M of agent should be maintained on the implant or barrier surface.

Other examples of agents which can be used include those having a mid-potency in the assays described herein (approximately 100-500 nM IC₅₀ range) such as loteprednol etabonate, juglone, prednisolone, puromycin, 3-BAABE, cladribine, and mannose-6-phosphate. For mid-potency drugs, the total dose typically should not to exceed 500 mg (range of 1.0 μg to 500 mg) and preferably 1 μg to 200 mg; dose per unit area of 0.01 μg-200 μg per mm², preferably 0.1 μg/mm²-40 μg/mm²; and minimum concentration of 10⁻⁸-10⁻⁴ M of agent should be maintained on the implant or barrier surface.

Other examples of agents which can be used include those having a low potency in the assays described herein (approximately 500-1000 nm range IC₅₀ range) such as 5-azacytidine, Ly333531 (ruboxistaurin), and simvastatin. For low potency drugs, the total dose typically should not exceed 1000 mg (range of 0.1 μg to 1000 mg), preferably 1 μg to 500 mg; dose per unit area of 0.01 μg-500 μg per mm²; preferably 0.1 μg/mm²-100 μg/mm²; and minimum concentration of 10⁻⁸-10⁻⁴ M of agent should to be maintained on the implant or barrier surface.

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

For greater clarity, several specific cardiac rhythm management devices and treatments will be described in greater detail below.

(1) Cardiac Pacemakers

In one aspect, the subject agents or compositions may be infiltrated into tissue adjacent to a cardiac pacemaker. The subject compositions may contain a therapeutic agent (e.g., an anti-scarring and/or anti-infective agent).

Cardiac rhythm abnormalities are extremely common in clinical practice and the incidence increases in frequency with both age and the presence of underlying coronary artery disease or myocardial infarction. A litany of arrythmias exists, but they are generally categorized into conditions where the heart beats too slowly (bradyarrythmias—such heart block, sinus node dysfunction) or too quickly (tachyarrhythmias—such as atrial fibrillation, WPW syndrome, ventricular fibrillation). A pacemaker functions by sending an electrical pulse (a pacing pulse) that travels via an electrical lead to the electrode (at the tip of the lead) which delivers an electrical impulse to the heart that initiates a heartbeat. The leads and electrodes can be located in one chamber (either the right atrium or the right ventricle—called single-chamber pacemakers) or there can be electrodes in both the right atrium and the right ventricle (called dual-chamber pacemakers). Electrical leads may be implanted on the exterior of the heart (e.g., epicardial leads) by a surgical procedure, or they can be connected to the endocardial surface of the heart via a catheter, guidewire or stylet. In some pacemakers, the device assumes the rhythm generating function of the heart and fires at a regular rate. In other pacemakers, the device merely augments the heart's own pacing function and acts “on demand” to provide pacing assistance as required (called “adaptive-rate” pacemakers); the pacemaker receives feedback on heart rhythm (and hence when to fire) from an electrode sensor located on the lead. Other pacemakers, called rate responsive pacemakers, have special sensors that detect changes in body activity (such as movement of the arms and legs, respiratory rate) and adjust pacing up or down accordingly.

Numerous pacemakers and pacemaker leads are suitable for use in this invention. For example, the pacing lead may have an increased resistance to fracture by being composed of an elongated coiled conductor mounted within a lumen of a lead body whereby it may be coupled electrically to a stranded conductor. See e.g., U.S. Pat. Nos. 6,061,598 and 6,018,683. The pacing lead may have a coiled conductor with an insulated sheath, which has a resistance to crush fatigue in the region between the rib and clavicle. See e.g., U.S. Pat. No. 5,800,496. The pacing lead may be expandable from a first, shorter configuration to a second, longer configuration by being composed of slideable inner and outer overlapping tubes containing a conductor. See e.g., U.S. Pat. No. 5,897,585. The pacing lead may have the means for temporarily making the first portion of the lead body stiffer by using a magnet-rheologic fluid in a cavity that stiffens when exposed to a magnetic field. See e.g., U.S. Pat. No. 5,800,497. The pacing lead may be a coil configuration composed of a plurality of wires or wire bundles made from a duplex titanium alloy. See e.g., U.S. Pat. No. 5,423,881. The pacing lead may be composed of a wire wound in a coil configuration with the wire composed of stainless steel having a composition of at least 22% nickel and 2% molybdenum. See e.g., U.S. Pat. No. 5,433,744. Other pacing leads are described in, e.g., U.S. Pat. Nos. 6,489,562; 6,289,251 and 5,957,967.

In another aspect, the electrical lead used in the practice of this invention may have an active fixation element for attachment to tissue. For example, the electrical lead may have a rigid fixation helix with microgrooves that are dimensioned to minimize the foreign body response following implantation. See e.g., U.S. Pat. No. 6,078,840. The electrical lead may have an electrode/anchoring portion with a dual tapered self-propelling spiral electrode for attachment to vessel wall. See e.g., U.S. Pat. No. 5,871,531. The electrical lead may have a rigid insulative electrode head carrying a helical electrode. See e.g., U.S. Pat. No. 6,038,463. The electrical lead may have an improved anchoring sleeve designed with an introducer sheath to minimize the flow of blood through the sheath during introduction. See e.g., U.S. Pat. No. 5,827,296. The electrical lead may be composed of an insulated electrical conductive portion and a lead-in securing section having a longitudinally rigid helical member which may be screwed into tissue. See e.g., U.S. Pat. No. 4,000,745.

Suitable leads for use in the practice of this invention also include multi-polar leads with multiple electrodes connected to the lead body. For example, the electrical lead may be a multi-electrode lead whereby the lead has two internal conductors and three electrodes with two electrodes coupled by a capacitor integral with the lead. See e.g., U.S. Pat. No. 5,824,029. The electrical lead may be a lead body with two straight sections and a bent third section with associated conductors and electrodes whereby the electrodes are bipolar. See e.g., U.S. Pat. No. 5,995,876. In another aspect, the electrical lead may be implanted by using a catheter, guidewire or stylet. For example, the electrical lead may be composed of an elongated insulative lead body having a lumen with a conductor mounted within the lead body and a resilient seal having an expandable portion through which a guidewire may pass. See e.g., U.S. Pat. No. 6,192,280.

Cardiac pacemakers, which may benefit from having the subject composition infiltrated into adjacent tissue according to the present invention, include commercially available products. Commercially available pacemakers suitable for the practice of the invention include the KAPPA SR 400 Series single-chamber rate-responsive pacemaker system, the KAPPA DR 400 Series dual-chamber rate-responsive pacemaker system, the KAPPA 900 and 700 Series single-chamber rate-responsive pacemaker system, and the KAPPA 900 and 700 Series dual-chamber rate-responsive pacemaker system by Medtronic, Inc. Medtronic pacemaker systems utilize a variety leads including the CAPSURE Z Novus, CAPSUREFIX Novus, CAPSUREFIX, CAPSURE SP Novus, CAPSURE SP, CAPSURE EPI and the CAPSURE VDD which may benefit from having the subject composition infiltrated into adjacent tissue. Pacemaker systems and associated leads that are made by Medtronic are described in, e.g., U.S. Pat. Nos. 6,741,893; 5,480,441; 5,411,545; 5,324,310; 5,265,602; 5,265,601; 5,241,957 and 5,222,506. Medtronic also makes a variety of steroid-eluting leads including those described in, e.g., U.S. Pat. Nos. 5,987,746; 6,363,287; 5,800,470; 5,489,294; 5,282,844 and 5,092,332. The INSIGNIA single-chamber and dual-chamber system, PULSAR MAX II DR dual-chamber adaptive-rate pacemaker, PULSAR MAX II SR single-chamber adaptive-rate pacemaker, DISCOVERY II DR dual-chamber adaptive-rate pacemaker, DISCOVERY II SR single-chamber adaptive-rate pacemaker, DISCOVERY II DDD dual-chamber pacemaker, and the DISCOVERY II SSI dingle-chamber pacemaker systems made by Guidant Corp. (Indianapolis, Ind.) are also suitable pacemaker systems for the practice of this invention. Once again, the leads from the Guidant pacemaker systems may benefit from having the subject composition infiltrated into adjacent tissue. Pacemaker systems and associated leads that are made by Guidant are described in, e.g., U.S. Pat. Nos. 6,473,648; 6,345,204; 6,321,122; 6,152,954; 5,769,881; 5,284,136; 5,086,773 and 5,036,849. The AFFINITY DR, AFFINITY VDR, AFFINITY SR, AFFINITY DC, ENTITY, IDENTITY, IDENTITY ADX, INTEGRITY, INTEGRITY μDR, INTEGRITY ADx, MICRONY, REGENCY, TRILOGY, and VERITY ADx, pacemaker systems and leads from St. Jude Medical, Inc. (St. Paul, Minn.) may also be suitable for use with the present invention to improve electrical transmission and sensing by the pacemaker leads. Pacemaker systems and associated leads that are made by St. Jude Medical are described in, e.g., U.S. Pat. Nos. 6,763,266; 6,760,619; 6,535,762; 6,246,909; 6,198,973; 6,183,305; 5,800,468 and 5,716,390. Alternatively, the fibrosis-inhibiting agent may be infiltrated into the region around the electrode-cardiac muscle interface under the present invention. It should be obvious to one of skill in the art that commercial pacemakers not specifically sited as well as next-generation and/or subsequently developed commercial pacemaker products are to be anticipated and are suitable for use under the present invention.

Regardless of the specific design features, for pacemakers to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. Pacemaker leads having the subject compositions infiltrated into tissue adjacent to the electrode-tissue and/or sensor-tissue interface, can increase the efficiency of impulse transmission and rhythm sensing, thereby increasing efficacy and battery longevity. Pacemaker leads may also benefit from release of a therapeutic agent able to prevent or inhibit infection in the vicinity of the implant site. Cardiac pacemakers and/or leads having the subject composition comprising an anti-scarring agent and/or anti-infective agent infiltrated into tissue adjacent to where the cardiac pacemaker and/or leads are or will be implanted. In another aspect, the present invention provides pacemaker leads having the subject composition comprising an anti-scarring agent and/or anti-infective agent infiltrated into tissue adjacent to the myocardial tissue where the lead will be implanted.

In another aspect, the present invention provides cardiac pacemakers having the subject compositions infiltrated into adjacent tissue, where the subject compositions may include a therapeutic agent (e.g., an anti-scarring and/or anti-infective agent). Numerous polymeric and non-polymeric delivery systems for use in connection with cardiac pacemakers have been described above.

Therapeutic agents or pharmaceutical compositions may be infiltrated around implanted cardiac pacemakers by applying the composition directly and/or indirectly into and/or onto (a) tissue adjacent to the cardiac pacemaker; (b) the vicinity of the cardiac pacemaker-tissue interface; (c) the region around the cardiac pacemaker; and (d) tissue surrounding the cardiac pacemaker. Methods for infiltrating the subject compositions into tissue adjacent to a cardiac pacemaker include delivering the composition: (a) to the surface of the cardiac pacemaker (e.g., as an injectable, paste, gel or mesh) during the implantation procedure; (b) to the surface of the tissue (e.g., as an injectable, paste, gel, in situ forming gel or mesh) immediately prior to, or during, implantation of the cardiac pacemaker; (c) to the surface of the cardiac pacemaker and/or the tissue surrounding the implanted cardiac pacemaker (e.g., as an injectable, paste, gel, in situ forming gel or mesh) immediately after the implantation of the cardiac pacemaker; (d) by topical application of the composition into the anatomical space where the cardiac pacemaker may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the cardiac pacemaker as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to cardiac pacemakers may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As cardiac pacemakers are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(2) Implantable Cardioverter Defibrillator (ICD) Systems

In one aspect, the subject agents or compositions may be infiltrated into tissue adjacent to an implantable cardioverter defibrillator (ICD) system. The subject compositions may contain a therapeutic agent (e.g., an anti-scarring and/or anti-infective agent).

Implantable cardioverter defibrillator (ICD) systems are similar to pacemakers (and many include a pacemaker system), but are used for the treatment of tachyarrhythmias such as ventricular tachycardia or ventricular fibrillation. An ICD consists of a mini-computer powered by a battery which is connected to a capacitor to helps the ICD charge and store enough energy to deliver therapy when needed. The ICD uses sensors to monitor the activity of the heart and the computer analysizes the data to determine when and if an arrhythmia is present. An ICD lead, which is inserted via a vein (called “transvenous” leads; in some systems the lead is implanted surgically—called an epicardial lead—and sewn onto the surface of the heart), connects into the pacing/computer unit. The lead, which is usually placed in the right ventricle, consists of an insulated wire and an electrode tip that contains a sensing component (to detect cardiac rhythm) and a shocking coil. A single-chamber ICD has one lead placed in the ventricle which defibrillates and paces the ventricle, while a dual-chamber ICD defibrillates the ventricle and paces the atrium and the ventricle. In some cases, an additional lead is required and is placed under the skin next to the rib cage or on the surface of the heart. In patients who require tachyarrhythmia management of the ventricle and atrium, a second coil is placed in the atrium to treat atrial tachycardia, atrial fibrillation and other arrhythmias. If a tachyarrhythmia is detected, a pulse is generated and propagated via the lead to the shocking coil which delivers a charge sufficient to depolarize the muscle and cardiovert or defibrillate the heart.

Several ICD systems have been described and are suitable for use in the practice of this invention. Representative examples of ICD's and associated components are described in U.S. Pat. Nos. 3,614,954, 3,614,955, 4,375,817, 5,314,430, 5,405,363, 5,607,385, 5,697,953, 5,776,165, 6,067,471, 6,169,923, and 6,152,955. Several ICD leads are suitable for use in the practice of this invention. For example, the defibrillator lead may be a linear assembly of sensors and coils formed into a loop which includes a conductor system for coupling the loop system to a pulse generator. See e.g., U.S. Pat. No. 5,897,586. The defibrillator lead may have an elongated lead body with an elongated electrode extending from the lead body, such that insulative tubular sheaths are slideably mounted around the electrode. See e.g., U.S. Pat. No. 5,919,222. The defibrillator lead may be a temporary lead with a mounting pad and a temporarily attached conductor with an insulative sleeve whereby a plurality of wire electrodes are mounted. See e.g., U.S. Pat. No. 5,849,033. Other defibrillator leads are described in, e.g., U.S. Pat. No. 6,052,625. In another aspect, the electrical lead may be adapted to be used for pacing, defibrillating or both applications. For example, the electrical lead may be an electrically insulated, elongated, lead body sheath enclosing a plurality of lead conductors that are separated from contacting one another. See e.g., U.S. Pat. No. 6,434,430. The electrical lead may be composed of an inner lumen adapted to receive a stiffening member (e.g., guide wire) that delivers fluoro-visible media. See e.g., U.S. Pat. No. 6,567,704. The electrical lead may be a catheter composed of an elongated, flexible, electrically nonconductive probe contained within an electrically conductive pathway that transmits electrical signals, including a defibrillation pulse and a pacer pulse, depending on the need that is sensed by a governing element. See e.g., U.S. Pat. No. 5,476,502. The electrical lead may have a low electrical resistance and good mechanical resistance to cyclical stresses by being composed of a conductive wire core formed into a helical coil covered by a layer of electrically conductive material and an electrically insulating sheath covering. See e.g., U.S. Pat. No. 5,330,521. Other electrical leads that may be adapted for use in pacing and/or defibrillating applications are described in, e.g., U.S. Pat. Nos. 6,556,873.

ICDs, which may benefit from having the subject composition infiltrated into adjacent tissue according to the present invention, include commercially available products. Commercially available ICDs suitable for the practice of the invention include the GEM III DR dual-chamber ICD, GEM III VR ICD, GEM II ICD, GEM ICD, GEM III AT atrial and ventricular arrhythmia ICD, JEWEL AF dual-chamber ICD, MICRO JEWEL ICD, MICRO JEWEL II ICD, JEWEL Plus ICD, JEWEL ICD, JEWEL ACTIVE CAN ICD, JEWEL PLUS ACTIVE CAN ICD, MAXIMO DR ICD, MAXIMO VR ICD, MARQUIS DR ICD, MARQUIS VR system, and the INTRINSIC dual-chamber ICD by Medtronic, Inc. Medtronic ICD systems utilize a variety leads including the SPRINT FIDELIS, SPRINT QUATRO SECURE steroid-eluting bipolar lead, Subcutaneous Lead System Model 6996SQ subcutaneous lead, TRANSVENE 6937A transvenous lead, and the 6492 Unipolar Atrial Pacing Lead which may benefit from having the subject composition infiltrated into adjacent tissue. ICD systems and associated leads that are made by Medtronic are described in, e.g., U.S. Pat. Nos. 6,038,472; 5,849,031; 5,439,484; 5,314,430; 5,165,403; 5,099,838 and 4,708,145. The VITALITY 2 DR dual-chamber ICD, VITALITY 2 VR single-chamber ICD, VITALITY AVT dual-chamber ICD, VITALITY DS dual-chamber ICD, VITALITY DS VR single-chamber ICD, VITALITY EL dual-chamber ICD, VENTAK PRIZM 2 DR dual-chamber ICD, and VENTAK PRIZM 2 VR single-chamber ICD systems made by Guidant Corp. are also suitable ICD systems for the practice of this invention. Once again, the leads from the Guidant ICD systems may benefit from having the subject composition infiltrated into adjacent tissue. Guidant sells the FLEXTEND Bipolar Leads, EASYTRAK Lead System, FINELINE Leads, and ENDOTAK RELIANCE ICD Leads. ICD systems and associated leads that are made by Guidant are described in, e.g., U.S. Pat. Nos. 6,574,505; 6,018,681; 5,697,954; 5,620,451; 5,433,729; 5,350,404; 5,342,407; 5,304,139 and 5,282,837. Biotronik, Inc. (Germany) sells the POLYROX Endocardial Leads, KENTROX SL Quadripolar ICD Leads, AROX Bipolar Leads, and MAPOX Bipolar Epicardial Leads (see e.g., U.S. Pat. Nos. 6,449,506; 6,421,567; 6,418,348; 6,236,893 and 5,632,770). The CONTOUR MD ICD, PHOTON p DR ICD, PHOTON μ VR ICD, ATLAS+ HF ICD, EPIC HF ICD, EPIC+ HF ICD systems and leads from St. Jude Medical may also benefit from having the subject composition infiltrated into adjacent tissue to improve electrical transmission and sensing by the ICD leads (see e.g., U.S. Pat. Nos. 5,944,746; 5,722,994; 5,662,697; 5,542,173; 5,456,706 and 5,330,523). Alternatively, the fibrosis-inhibiting agent may be infiltrated into the region around the electrode-cardiac muscle interface under the present invention. It should be obvious to one of skill in the art that commercial ICDs not specifically sited as well as next-generation and/or subsequently developed commercial ICD products are to be anticipated and are suitable for use under the present invention.

Regardless of the specific design features, for ICDs to be effective in the management of cardiac rhythm disorders, the leads must be accurately positioned adjacent to the targeted cardiac muscle tissue. If excessive scar tissue growth or extracellular matrix deposition occurs around the leads, efficacy can be compromised. ICD leads having the subject compositions infiltrated into tissue adjacent to the electrode-tissue and/or sensor-tissue interface, can increase the efficiency of impulse transmission and rhythm sensing, thereby increasing efficacy, preventing inappropriate cardioversion, and improving battery longevity. ICDs may also benefit from release of a therapeutic agent able to prevent or inhibit infection in the vicinity of the implant site. In one aspect, the device includes ICDs and/or leads having the subject composition comprising an anti-scarring agent and/or anti-infective agent infiltrated into tissue adjacent to where the ICD and/or leads are or will be implanted. In another aspect, the present invention provides ICD leads having the subject composition comprising an anti-scarring agent and/or anti-infective agent infiltrated into tissue adjacent to the myocardial tissue surrounding the lead.

In another aspect, the present invention provides ICDs having the subject compositions infiltrated into adjacent tissue, where the subject compositions may include a therapeutic agent (e.g., an anti-scarring and/or anti-infective agent). Numerous polymeric and non-polymeric delivery systems for use in connection with ICDs have been described above.

Therapeutic agents or pharmaceutical compositions may be infiltrated around implanted ICDs by applying the composition directly and/or indirectly into and/or onto (a) tissue adjacent to the ICD; (b) the vicinity of the ICD-tissue interface; (c) the region around the ICD; and (d) tissue surrounding the ICD. Methods for infiltrating the subject compositions into tissue adjacent to a ICD include delivering the composition: (a) to the surface of the ICD (e.g., as an injectable, paste, gel or mesh) during the implantation procedure; (b) to the surface of the tissue (e.g., as an injectable, paste, gel, in situ forming gel or mesh) immediately prior to, or during, implantation of the ICD; (c) to the surface of the ICD and/or the tissue surrounding the implanted ICD (e.g., as an injectable, paste, gel, in situ forming gel or mesh) immediately after the implantation of the ICD; (d) by topical application of the composition into the anatomical space where the ICD may be placed (particularly useful for this embodiment is the use of polymeric carriers which release the therapeutic agent over a period ranging from several hours to several weeks—fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the ICD as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to ICDs may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As ICDs are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

(3) Vagus Nerve Stimulation for the Treatment of Arrhythmia

In one aspect, the subject agents or compositions may be infiltrated into tissue adjacent to a vagal nerve stimulation (VNS) device. The subject compositions may contain a therapeutic agent (e.g., an anti-scarring and/or anti-infective agent).

A neurostimulation device may also be used to stimulate the vagus nerve and affect the rhythm of the heart. Since the vagus nerve provides innervation to the heart, including the conduction system (including the SA node), stimulation of the vagus nerve may be used to treat conditions such as supraventricular arrhythmias, angina pectoris, atrial tachycardia, atrial flutter, atrial fibrillation and other arrhythmias that result in low cardiac output.

As described above, in VNS a bipolar electrical lead is surgically implanted such that it transmits electrical stimulation from the pulse generator to the left vagus nerve in the neck. The pulse generator is an implanted, lithium carbon monofluoride battery-powered device that delivers a precise pattern of stimulation to the vagus nerve. The pulse generator can be programmed (using a programming wand) by the cardiologist to treat a specific arrhythmia.

Products such as these have been described, for example, in U.S. Pat. Nos. 6,597,953 and 6,615,085. For example, the neurostimulator may be a vagal-stimulation apparatus which generates pulses at a frequency that varies automatically based on the excitation rates of the vagus nerve. See e.g., U.S. Pat. Nos. 5,916,239 and 5,690,681. The neurostimulator may be an apparatus that detects characteristics of tachycardia based on an electrogram and delivers a preset electrical stimulation to the nervous system to depress the heart rate. See e.g., U.S. Pat. No. 5,330,507. The neurostimulator may be an implantable heart stimulation system composed of two sensors, one for atrial signals and one for ventricular signals, and a pulse generator and control unit, to ensure sympatho-vagal stimulation balance. See e.g., U.S. Pat. No. 6,477,418. The neurostimulator may be a device that applies electrical pulses to the vagus nerve at a programmable frequency that is adjusted to maintain a lower heart rate. See e.g., U.S. Pat. No. 6,473,644. The neurostimulator may provide electrical stimulation to the vagus nerve to induce changes to electroencephalogram readings as a treatment for epilepsy, while controlling the operation of the heart within normal parameters. See e.g., U.S. Pat. No. 6,587,727.

VNS devices, which may benefit from having the subject composition infiltrated into adjacent tissue according to the present invention, include commercially available products. A commercial example of a VNS system is the product produced by Cyberonics Inc. that consists of the Model 300 and Model 302 leads, the Model 101 and Model 102R pulse generators, the Model 201 programming wand and Model 250 programming software, and the Model 220 magnets. These products manufactured by Cyberonics, Inc. may be described, for example, in U.S. Pat. Nos. 5,928,272; 5,540,730 and 5,299,569.

Regardless of the specific design features, for vagal nerve stimulation to be effective in arrhythmias, the leads must be accurately positioned adjacent to the left vagus nerve. If excessive scar tissue growth or extracellular matrix deposition occurs around the VNS leads, this can reduce the efficacy of the device. VNS devices having the subject compositions infiltrated into tissue adjacent to the electrode-tissue interface can increase the efficiency of impulse transmission and increase the duration that these devices function clinically. VNS devices may also benefit from release of a therapeutic agent able to prevent or inhibit infection in the vicinity of the implant site. In one aspect, the device includes VNS devices and/or leads having the subject composition comprising an anti-scarring agent and/or anti-infective agent infiltrated into tissue adjacent to where the VNS device and/or leads are or will be implanted. In another aspect, the present invention provides leads having the subject composition comprising an anti-scarring agent and/or anti-infective agent infiltrated into tissue adjacent to the vagus nerve where the lead will be implanted.

In another aspect, the present invention provides VNS devices having the subject compositions infiltrated into adjacent tissue, where the subject compositions may include a therapeutic agent (e.g., an anti-scarring and/or anti-infective agent). Numerous polymeric and non-polymeric delivery systems for use in connection with VNS devices have been described above.

Therapeutic agents or pharmaceutical compositions may be infiltrated around implanted VNS devices by applying the composition directly and/or indirectly into and/or onto (a) tissue adjacent to the VNS device; (b) the vicinity of the VNS device-tissue interface; (c) the region around the VNS device; and (d) tissue surrounding the VNS device. Methods for infiltrating the subject compositions into tissue adjacent to a VNS device include delivering the composition: (a) to the surface of the VNS device (e.g., as an injectable, paste, gel or mesh) during the implantation procedure; (b) to the surface of the tissue (e.g., as an injectable, paste, gel, in situ forming gel or mesh) immediately prior to, or during, implantation of the VNS device; (c) to the surface of the VNS device and/or the tissue surrounding the implanted VNS device (e.g., as an injectable, paste, gel, in situ forming gel or mesh) immediately after the implantation of the VNS device; (d) by topical application of the composition into the anatomical space where the VNS device may be placed (particularly useful for this embodiment is the use of polymeric carriers which release the therapeutic agent over a period ranging from several hours to several weeks-fluids, suspensions, emulsions, microemulsions, microspheres, pastes, gels, microparticulates, sprays, aerosols, solid implants and other formulations which release the agent may be delivered into the region where the device may be inserted); (e) via percutaneous injection into the tissue surrounding the VNS device as a solution as an infusate or as a sustained release preparation; (f) by any combination of the aforementioned methods. Combination therapies (i.e., combinations of therapeutic agents and combinations with antithrombotic and/or antiplatelet agents) may also be used. In all cases it is understood that the subject compositions may be infiltrated into tissue adjacent to all or a portion of the device, including the device only, lead only, electrode only and/or a combination thereof.

According to one aspect, any fibrosis-inhibiting and/or anti-infective agent described above may be utilized in the practice of the present invention. In one aspect of the invention, the subject compositions infiltrated into tissue adjacent to VNS devices may be adapted to release an agent that inhibits one or more of the four general components of the process of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue). By inhibiting one or more of the components of fibrosis (or scarring), the overgrowth of granulation tissue may be inhibited or reduced.

Examples of fibrosis-inhibiting agents for use in the present invention include the following: ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin, as well as analogues and derivatives of the aforementioned.

The drug dose administered from the present compositions for prevention or inhibition of fibrosis in accordance with the present invention will depend on a variety of factors, including the type of formulation, the location of the treatment site, and the type of condition being treated. As VNS devices are made in a variety of configurations and sizes, the exact dose administered will also vary with device size, surface area and design. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single chemotherapeutic systemic dose application. In certain aspects, the anti-scarring agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-fibrosing agents, used alone or in combination, should be administered under the following dosing guidelines. The total amount (dose) of anti-scarring agent in the composition can be in the range of about 0.01 μg-10 μg, or about 10 μg-10 mg, or about 10 mg-250 mg, or about 250 mg-1000 mg, or about 1000 mg-2500 mg. The dose (amount) of anti-scarring agent per unit area of device or tissue surface to which the agent is applied may be in the range of about 0.01 μg/mm²-1 μg/mm², or about 1 μg/mm²-10 μg/mm², or about 10 μg/mm²-250 μg/mm², or about 250 μg/mm²-1000 μg/mm², or about 1000 μg/mm²-2500 μg/mm².

According to another aspect, any anti-infective agent described above may be used in the practice of the present invention. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

The drug dose administered from the present compositions for prevention or inhibition of infection in accordance with the present invention will depend on a variety of factors, including the type of formulation, the location of the treatment site, and the type of condition being treated. However, certain principles can be applied in the application of this art. Drug dose can be calculated as a function of dose per unit area (of the treatment site), total drug dose administered can be measured and appropriate surface concentrations of active drug can be determined. Drugs are to be used at concentrations that range from several times more than to 50%, 20%, 10%, 5%, or even less than 1% of the concentration typically used in a single anti-infective systemic dose application. In certain aspects, the anti-infective agent is released from the composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days.

The exemplary anti-infective agents, used alone or in combination, should be administered under the following dosing guidelines. The total amount (dose) of anti-infective agent in the composition can be in the range of about 0.01 μg-1 μg, or about 1 μg-10 μg, or about 10 μg-1 mg, or about 1 mg to 10 mg, or about 10 mg-100 mg, or about 100 mg to 250 mg, or about 250 mg-1000 mg. The dose (amount) of anti-infective agent per unit area of device or tissue surface to which the agent is applied may be in the range of about 0.01 μg/mm²-1 μg/mm², or about 1 μg/mm²-10 μg/mm², or about 10 μg/mm²-100 μg/mm², or about 100 μg/mm² to 250 μg/mm², or about 250 μg/mm²-1000 μg/mm². As different compositions will release the anti-infective agent at differing rates, the above dosing parameters should be utilized in combination with the release rate of the drug from the composition such that a minimum concentration of about 10⁻⁸ to 10⁻⁷, or about 10⁻⁷ to 10⁻⁶ about 10⁻⁶ to 10⁻⁵ or about 10⁻⁵ to 10⁻⁴ of the agent is maintained on the tissue surface.

It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate), quinolones, and/or podophylotoxins (e.g., etoposide) may be utilized to enhance the antibacterial activity of the composition.

Although numerous cardiac rhythm management (CRM) devices have been described above, all possess similar design features and cause similar unwanted fibrous tissue reactions following implantation and may introduce or promote infection in the area of the implant site. It should be obvious to one of skill in the art that commercial CRM devices not specifically sited above as well as next-generation and/or subsequently-developed commercial CRM products are to be anticipated and are suitable for use under the present invention. The CRM device, particularly the lead(s), must be positioned in a very precise manner to ensure that stimulation is delivered to the correct anatomical location within the atrium and/or ventricle. All, or parts, of a CRM device can migrate following surgery, or excessive scar tissue growth can occur around the implant, which can lead to a reduction in the performance of these devices. CRM devices having the subject compositions infiltrated into tissue adjacent to the electrode-tissue interface can be used to increase the efficacy and/or the duration of activity of the implant (particularly for fully-implanted, battery-powered devices). CRM devices may also benefit from release of a therapeutic agent able to prevent or inhibit infection in the vicinity of the implant site. In one aspect, the present invention provides CRM devices having the subject compositions infiltrated into adjacent tissue, where the subject compositions may include a therapeutic agent (e.g., an anti-scarring and/or anti-infective agent). These compositions can further include one or more fibrosis-inhibiting agents such that the overgrowth of granulation fibrous, or gliotic tissue is inhibited or reduced and/or one or more anti-infective agents such that infection in the vicinity of the implant site is inhibited or prevented.

4) Sustained-Release Preparations of Fibrosis-Inhibiting (or Gliosis-Inhibiting) Agents

As described previously, desired fibrosis-inhibiting (or gliosis-inhibiting) agents may be admixed with, blended with, conjugated to, or, otherwise modified to contain a polymer composition (which may be either biodegradable or non-biodegradable), or a non-polymeric composition, in order to release the therapeutic agent over a prolonged period of time. For many of the aforementioned embodiments, localized delivery as well as localized sustained delivery of the fibrosis-inhibiting (or gliosis-inhibiting) agent may be required. For example, a desired fibrosis-inhibiting (or gliosis-inhibiting) agent may be admixed with, blended with, conjugated to, or otherwise modified to contain a polymeric composition (which may be either biodegradable or non-biodegradable), or non-polymeric composition, in order to release the fibrosis-inhibiting (or gliosis-inhibiting) agent over a period of time. In certain aspects, the polymer composition may include a bioerodible or biodegradable polymer. Representative examples of biodegradable polymer compositions suitable for the delivery of fibrosis-inhibiting (or gliosis-inhibiting) agents include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), degradable polyesters (e.g., polyesters comprising the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), poly(hydroxyvaleric acid), polydioxanone, poly(ethylene terephthalate), poly(malic acid), poly(tartronic acid), poly(acrylamides), polyanhydrides, polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide (e.g., poly(ethylene glycol), methoxy poly(ethylene glycol), poly(propylene glycol), block copolymers of poly(ethylene oxide) and poly(propylene oxide) (e.g., PLURONIC and PLURONIC R polymers) and Y is a polyester (e.g., polyester comprising the residues of one or more of the monomers selected from lactide, lactic acid, glycolide, glycolic acid, ε-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one.), R is a multifunctional initiator and copolymers as well as blends thereof)) and their copolymers, branched polymers as well as blends thereof. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986)).

Representative examples of non-degradable polymers suitable for the delivery of fibrosis-inhibiting (or gliosis-inhibiting) agents include poly(ethylene-co-vinyl acetate) (“EVA”) copolymers, silicone rubber, acrylic polymers (polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), polyethylene, polypropylene, polyamides (nylon 6,6), polyurethanes (e.g., CHRONOFLEX AR and CHRONOFLEX AL (both from CardioTech International, Inc., Woburn, Mass.), BIONATE (Polymer Technology Group, Inc., Emergyville, Cali.), and PELLETHANE (Dow Chemical Company, Midland, Mich.)), poly(ester urethanes), poly(ether urethanes), poly(ester-urea), polyethers (poly(ethylene oxide), poly(propylene oxide), block copolymers based on ethylene oxide and propylene oxide (i.e., copolymers of ethylene oxide and propylene oxide polymers), such as the family of PLURONIC polymers available from BASF Corporation (Mount Olive, N.J.), and poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm Bull. 16(11):1164-1168, 1993; Thacharodi and Rao, Int'l J. Pharm. 120:115-118, 1995; Miyazaki et al., Int'l J. Pharm. 118:257-263, 1995).

Particularly preferred polymeric carriers include poly(ethylene-co-vinyl acetate), polyurethanes (e.g., CHRONOFLEX AR, CHRONOFLEX AL, BIONATE, PELLETHANE), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), silicone rubbers, nitrocellulose, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of fibrosis-inhibiting (or gliosis-inhibiting) agents include carboxylic polymers, polyacetates, polyacrylamides, polycarbonates, polyethers, polyesters, polyethylenes, polyvinylbutyrals, polysilanes, polyureas, polyurethanes, polyurethanes (e.g., CHRONOFLEX AR, CHRONOFLEX AL, BIONATE, AND PELLETHANE), polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

In one embodiment, all or a portion of the device is coated with a primer (bonding) layer and a drug release layer, as described in U.S. patent application entitled, “Stent with Medicated Multi-Layer Hybrid Polymer Coating,” filed Sep. 16, 2003 (U.S. Ser. No. 10/662,877).

In order to develop a hybrid polymer delivery system for targeted therapy, it is desirable to be able to control and manipulate the properties of the system both in terms of physical and drug release characteristics. The active agents can be imbibed into a surface hybrid polymer layer, or incorporated directly into the hybrid polymer coating solutions. Imbibing drugs into surface polymer layers is an efficient method for evaluating polymer-drug performance in the laboratory, but for commercial production it may be preferred for the polymer and drug to be premixed in the casting mixture. Greater efficacy can be achieved by combining the two elements in the coating mixtures in order to control the ratio of active agent to polymer in the coatings. Such ratios are important parameters to the final properties of the medicated layers, i.e., they allow for better control of active agent concentration and duration of pharmacological activity.

Typical polymers used in the drug-release system can include water-insoluble cellulose esters, various polyurethane polymers including hydrophilic and hydrophobic versions, hydrophilic polymers such as polyethylene glycol (PEG), polyethylene oxide (PEO), polyvinylpyrrolidone (PVP), PVP copolymers such as vinyl acetate, hydroxyethyl methacrylate (HEMA) and copolymers such as methylmethacrylate (PMMA-HEMA), and other hydrophilic and hydrophobic acrylate polymers and copolymers containing functional groups such as carboxyl and/or hydroxyl.

Cellulose esters such as cellulose acetate, cellulose acetate propionate, cellulose acetate butyrate, cellulose acetate phthalate, and cellulose nitrate may be used. In one aspect of the invention, the therapeutic agent is formulated with a cellulose ester. Cellulose nitrate is a preferred cellulose ester because of its compatibility with the active agents and its ability to impart non-tackiness and cohesiveness to the coatings. Cellulose nitrate has been shown to stabilize entrapped drugs in ambient and processing conditions. Various grades of cellulose nitrate are available and may be used in a coating on a electrical device, including cellulose nitrate having a nitrogen content=11.8-12.2%. Various viscosity grades, including 3.5, 0.5 or 0.25 seconds, may be used in order to provide proper rheological properties when combined with the coating solids used in these formulations. Higher or lower viscosity grades can be used. However, the higher viscosity grades can be more difficult to use because of their higher viscosities. Thus, the lower viscosity grades, such as 3.5, 0.5 or 0.25 seconds, are generally preferred. Physical properties such as tensile strength, elongation, flexibility, and softening point are related to viscosity (molecular weight) and can decrease with the lower molecular weight species, especially below the 0.25 second grades.

The cellulose derivatives comprise hydroglucose structures. Cellulose nitrate is a hydrophobic, water-insoluble polymer, and has high water resistance properties. This structure leads to high compatibility with many active agents, accounting for the high degree of stabilization provided to drugs entrapped in cellulose nitrate. The structure of nitrocellulose is given below:

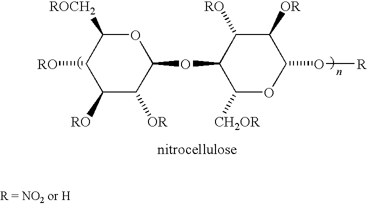

Cellulose nitrate is a hard, relatively inflexible polymer, and has limited adhesion to many polymers that are typically used to make medical devices. Also, control of drug elution dynamics is limited if only one polymer is used in the binding matrix. Accordingly, in one embodiment of the invention, the therapeutic agent is formulated with two or more polymers before being associated with the electrical device. In one aspect, the agent is formulated with both polyurethane ((e.g., CHRONOFLEX AR, CHRONOFLEX AL, BIONATE, and PELLETHANE) and cellulose nitrate to provide a hybrid polymer drug loaded matrix. Polyurethanes provide the hybrid polymer matrix with greater flexibility and adhesion to the electrical device, particularly when the connector has been pre-coated with a primer. Polyurethanes can also be used to slow or hasten the drug elution from coatings. Aliphatic, aromatic, polytetramethylene ether glycol, and polycarbonate are among the types of polyurethanes, which can be used in the coatings. In one aspect, an anti-scarring agent (e.g., ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), simvastatin,) may be incorporated into a carrier that includes a polyurethane and a cellulose derivative. A heparin complex, such as benzalkonium heparinate or tridodecylammonium heparinate), may optionally be included in the formulation.

From the structure below, it is possible to see how more or less hydrophilic polyurethane polymers may be created based on the number of hydrophilic groups contained in the polymer structures. In one aspect of the invention, the electrical device is associated with a formulation that includes therapeutic agent, cellulose ester, and a polyurethane that is water-insoluble, flexible, and compatible with the cellulose ester.

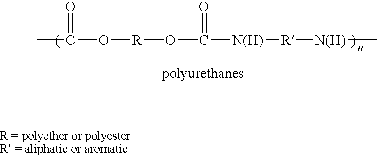

Polyvinylpyrrolidone (PVP) is a polyamide that possesses unusual complexing and colloidal properties and is essentially physiologically inert. PVP and other hydrophilic polymers are typically biocompatible. PVP may be incorporated into drug loaded hybrid polymer compositions in order to increase drug release rates. In one embodiment, the concentration of PVP that is used in drug loaded hybrid polymer compositions can be less than 20%. This concentration can not make the layers bioerodable or lubricious. In general, PVP concentrations from <1% to greater than 80% are deemed workable. In one aspect of the invention, the therapeutic agent that is associated with an electrical device is formulated with a PVP polymer.

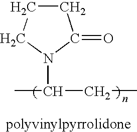

Acrylate polymers and copolymers including polymethylmethacrylate (PMMA) and polymethylmethacrylate hydroxyethyl methacrylate (PMMA/HEMA) are known for their biocompatibility as a result of their widespread use in contact and intraocular lens applications. This class of polymer generally provokes very little smooth muscle and endothelial cell growth, and very low inflammatory response (Bar). These polymers/copolymers are compatible with drugs and the other polymers and layers of the instant invention. Thus, in one aspect, the device is associated with a composition that comprises an anti-scarring agent as described above, and an acrylate polymer or copolymer.

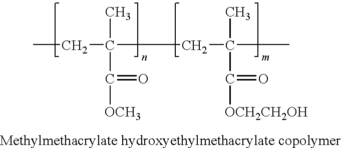

Representative examples of patents relating to drug-delivery polymers and their preparation include PCT Publication Nos. WO 98/19713, WO 01/17575, WO 01/41821, WO 01/41822, and WO 01/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Patent Application Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of fibrosis-inhibiting (or gliosis-inhibiting) agents.

Polymeric carriers for fibrosis-inhibiting (or gliosis-inhibiting) agents can be fashioned in a variety of forms, with desired release characteristics and/or with specific properties depending upon the device, composition or implant being utilized. For example, polymeric carriers may be fashioned to release a fibrosis-inhibiting (or gliosis-inhibiting) agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide Imonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inhibiting (or gliosis-inhibiting) agents can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intem. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and their preparation include U.S. Pat. Nos. 6,451,346; 6,201,072; 6,117,949; 6,004,573; 5,702,717; and 5,484,610 and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inhibiting (or gliosis-inhibiting) agents may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films and sprays.

Within certain aspects of the present invention, therapeutic compositions may be fashioned into particles having any size ranging from 50 nm to 500 μm, depending upon the particular use. These compositions can be in the form of microspheres, microparticles and/or nanoparticles. These compositions can be formed by spray-drying methods, milling methods, coacervation methods, W/O emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of paste or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inhibiting agents are particularly useful for application to the surface of tissues that will be in contact with the implant or device.

Within yet other aspects of the invention, the therapeutic compositions of the present invention may be formed as a film or tube. These films or tubes can be porous or non-porous. Such films or tubes are generally less than 5, 4, 3, 2, or 1 mm thick, or less than 0.75 mm, or less than 0.5 mm, or less than 0.25 mm, or, less than 0.10 mm thick. Films or tubes can also be generated of thicknesses less than 50 μm, 25 μm or 10 μm. Such films may be flexible with a good tensile strength (e.g., greater than 50, or greater than 100, or greater than 150 or 200 N/cm²), good adhesive properties (i.e., adheres to moist or wet surfaces), and have controlled permeability. Fibrosis-inhibiting agents contained in polymeric films are particularly useful for application to the surface of a device or implant as well as to the surface of tissue, cavity or an organ.

Within further aspects of the present invention, polymeric carriers are provided which are adapted to contain and release a hydrophobic fibrosis-inhibiting (or gliosis-inhibiting) compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic fibrosis-inhibiting (or gliosis-inhibiting) compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan, hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inhibiting (or gliosis-inhibiting) agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible light, UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977, U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975, and U.S. Patent Application Publication Nos. 2002/012796A1, 2002/0127266A1, 2002/0151650A1, 2003/0104032A1, 2002/0091229A1, and 2003/0059906A1.

As mentioned elsewhere herein, the present invention provides for polymeric crosslinked matrices, and polymeric carriers, that may be used to assist in the prevention of the formation or growth of fibrous connective tissue or glial tissue. The composition may contain and deliver fibrosis-inhibiting (or gliosis-inhibiting) agents in the vicinity of the medical device. The following compositions are particularly useful when it is desired to infiltrate around the device, with or without a fibrosis-inhibiting agent. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a device in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

### First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifunctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include: (i) synthetic polypeptides that have been synthesized to contain two or more primary amino groups or thiol groups; and (ii) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), di(2-aminoethyl)amine (HN—(CH₂—CH₂—NH₂)₂), and tris(2-aminoethyl)amine (N—(CH₂—CH₂—NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers which are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

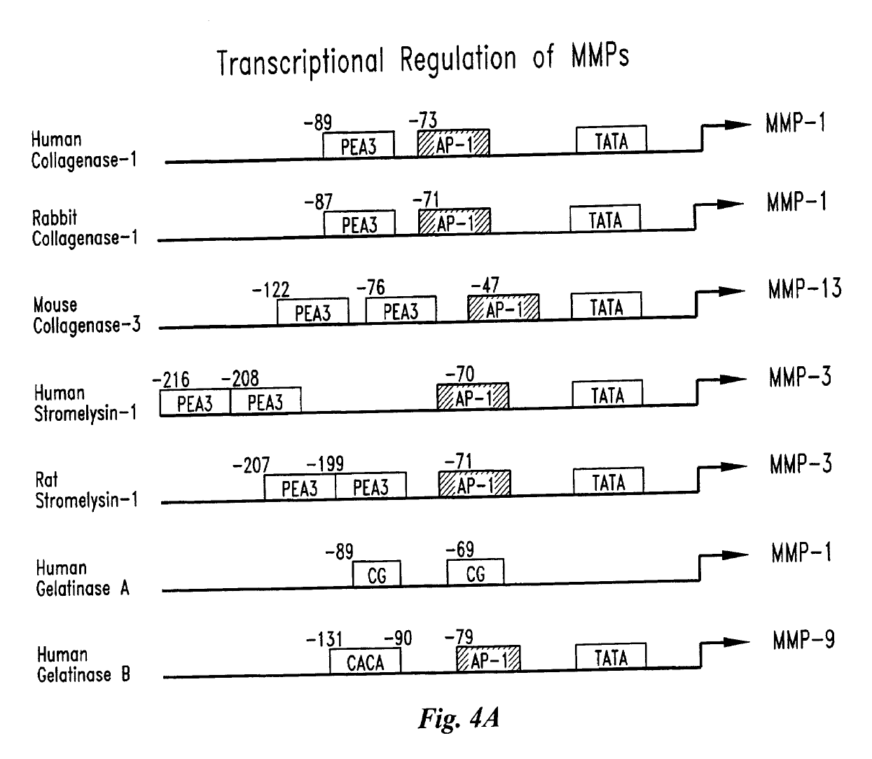

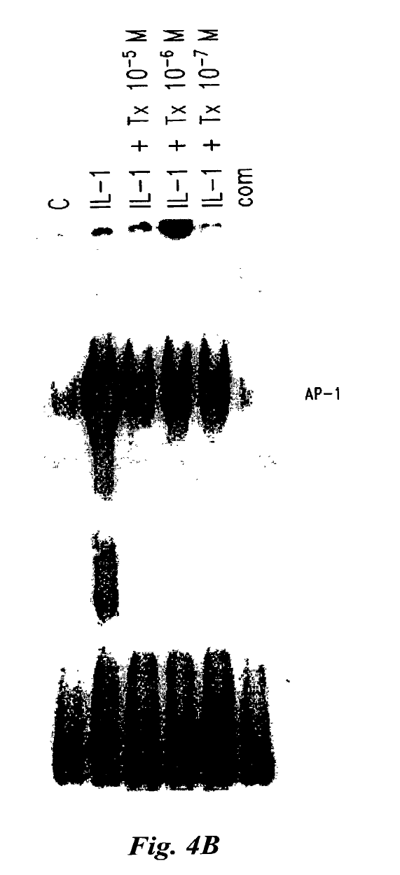

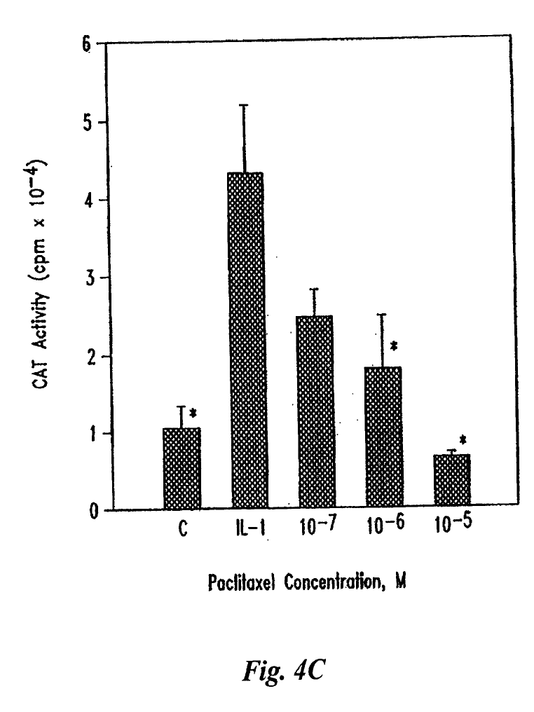

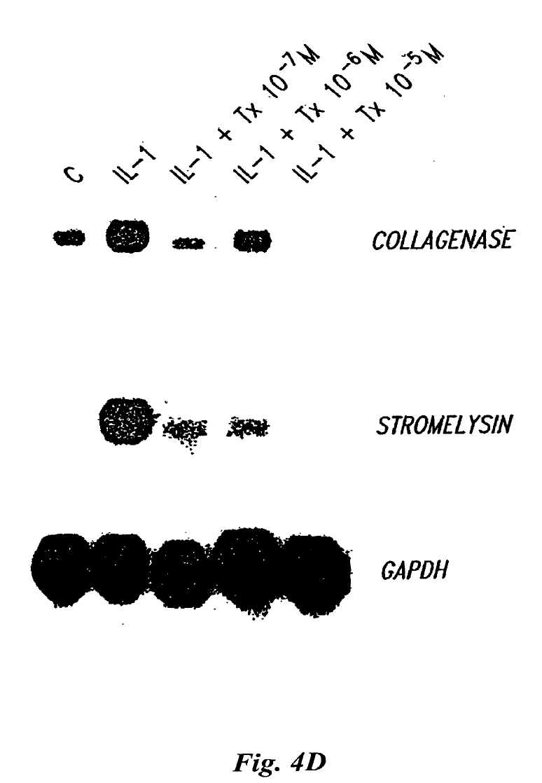

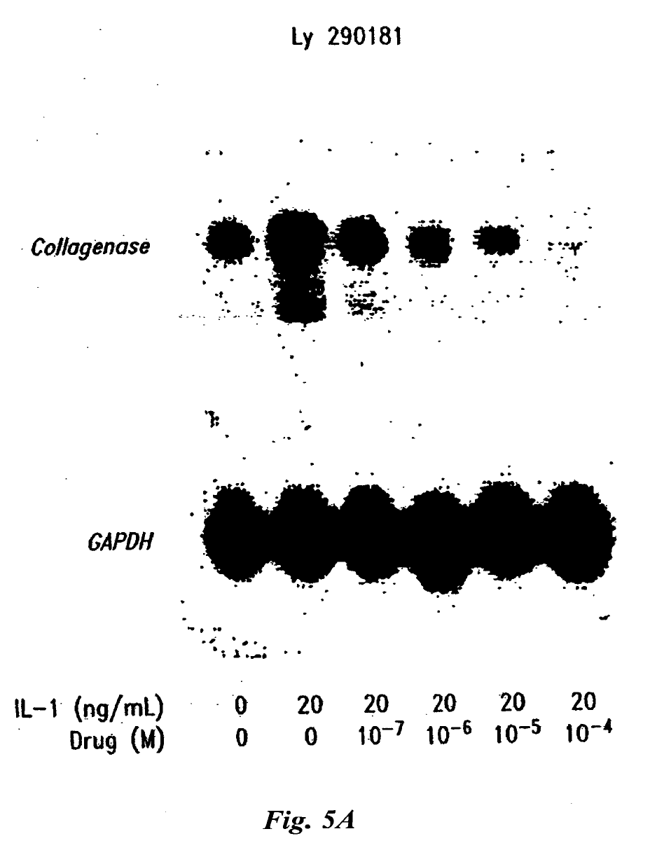

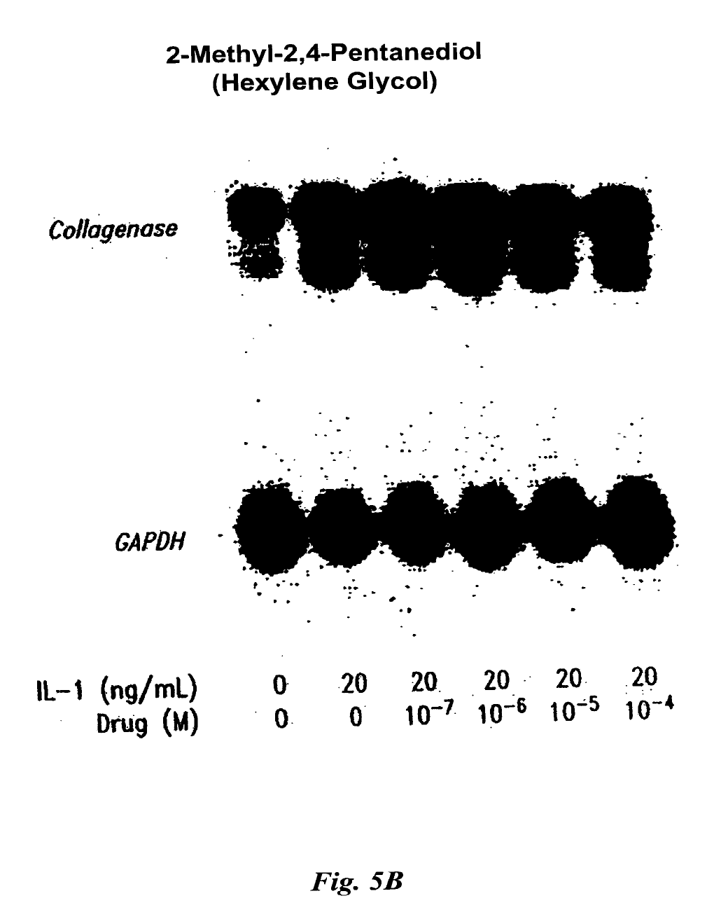

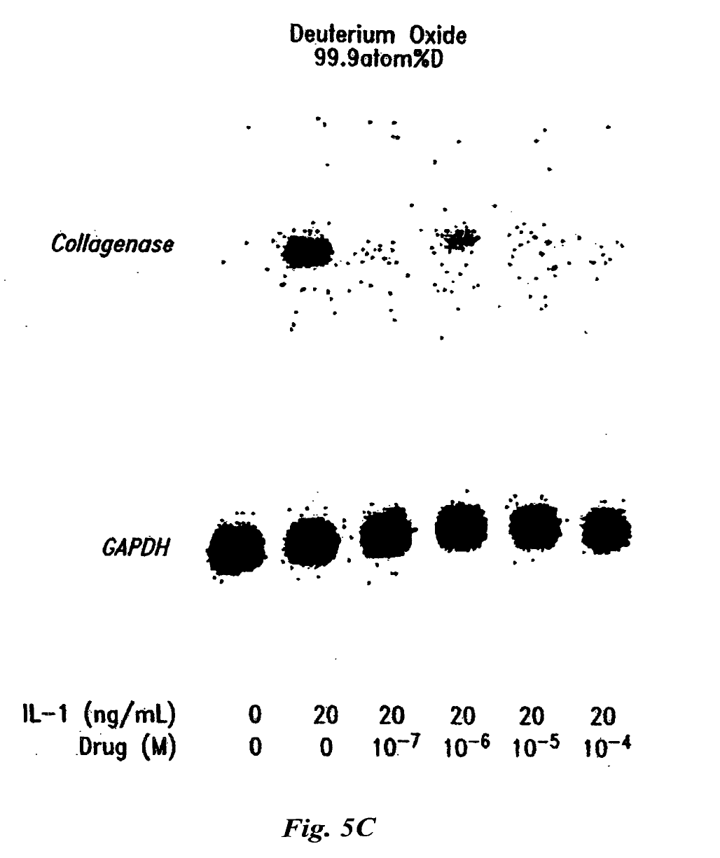

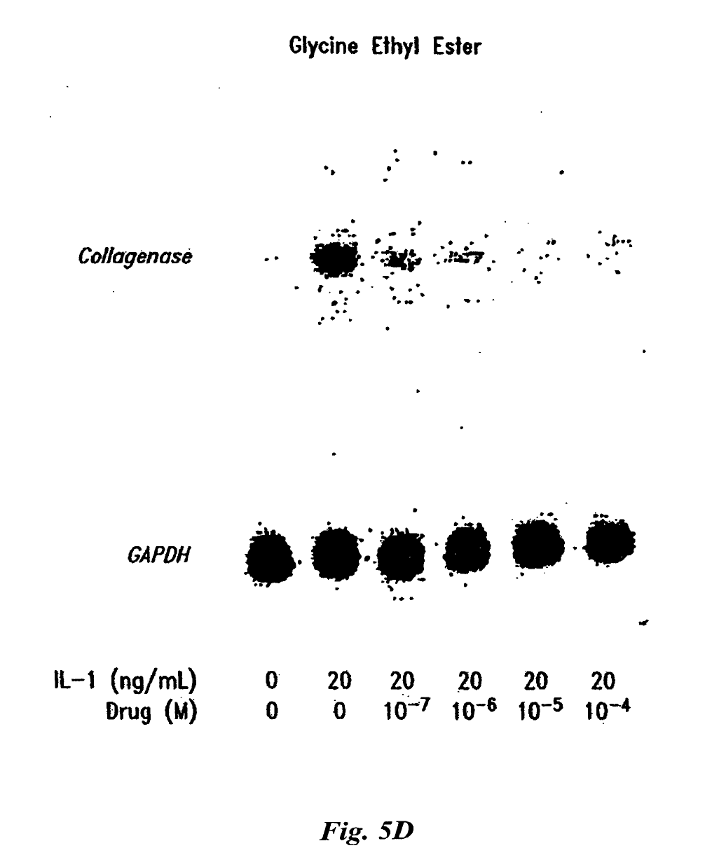

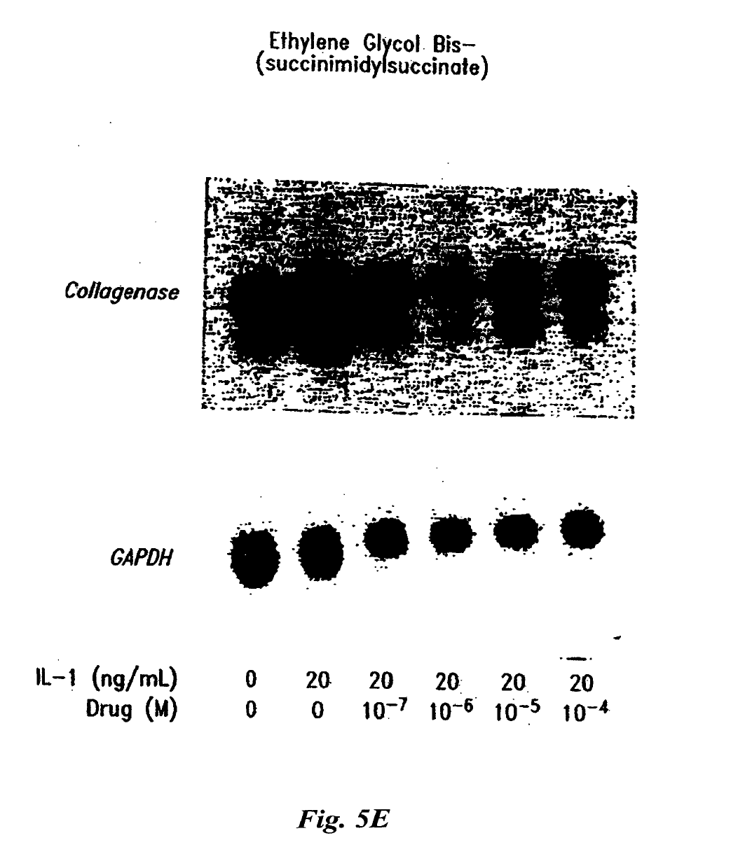

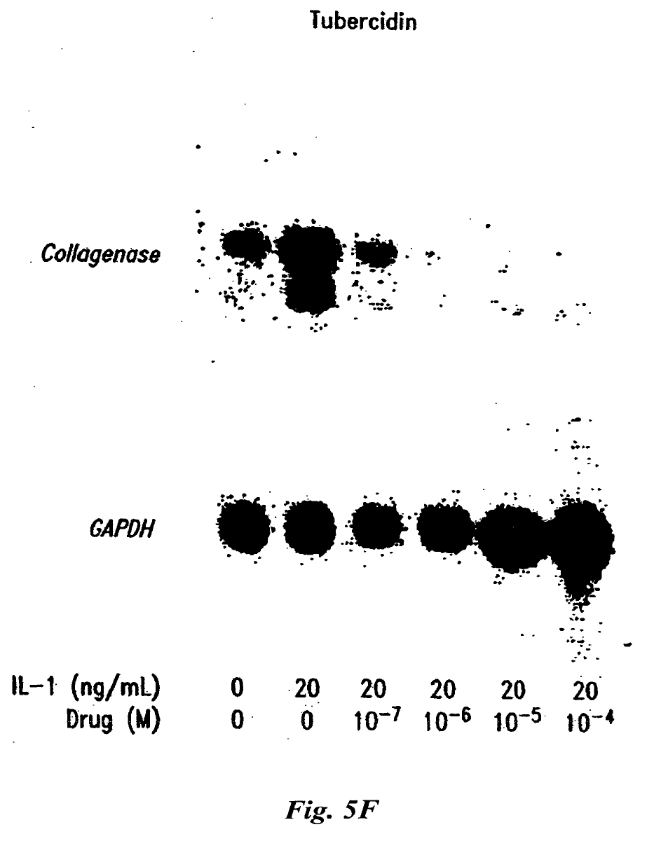

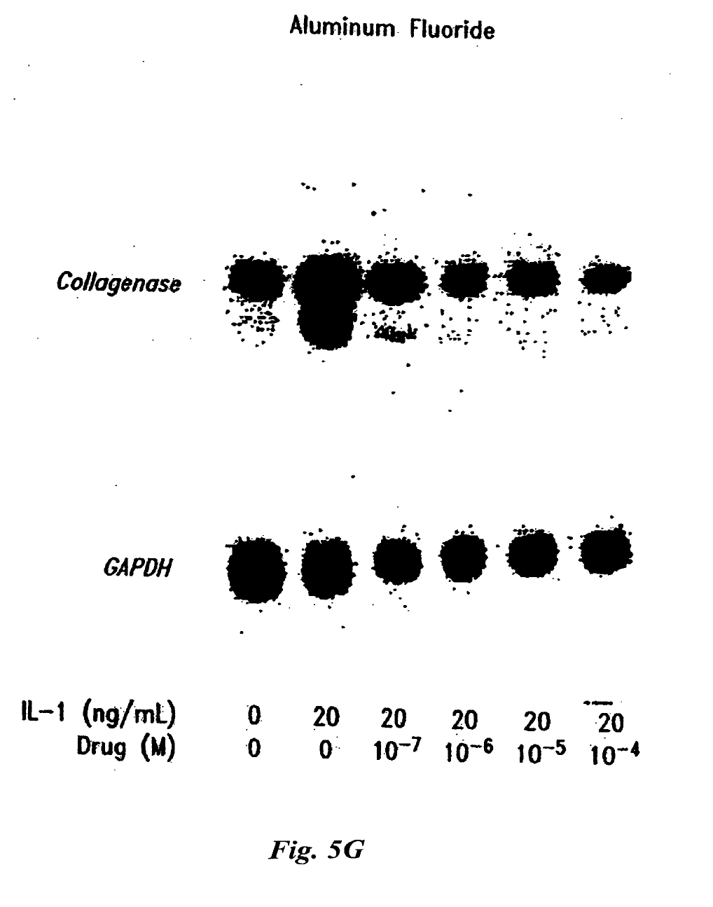

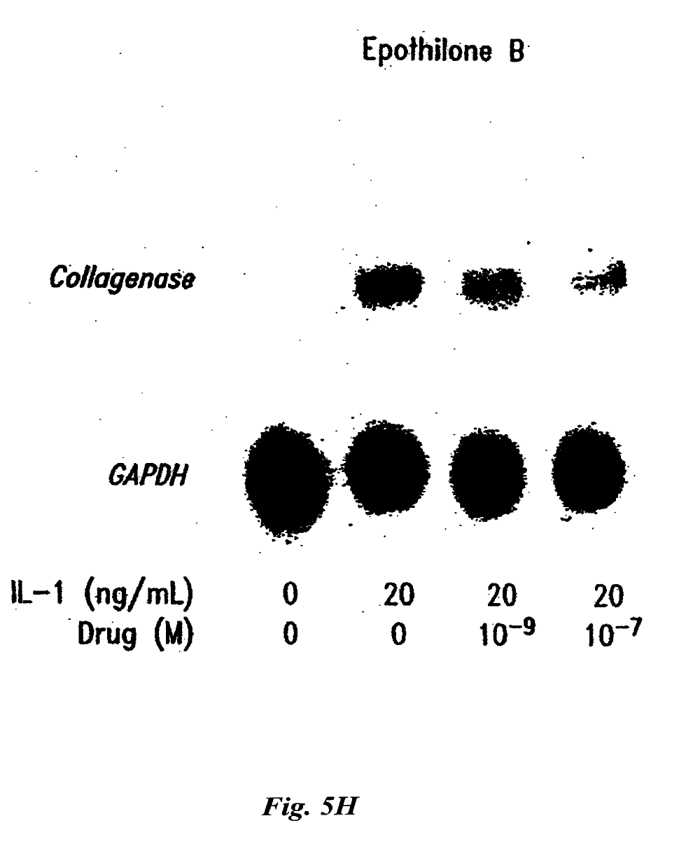

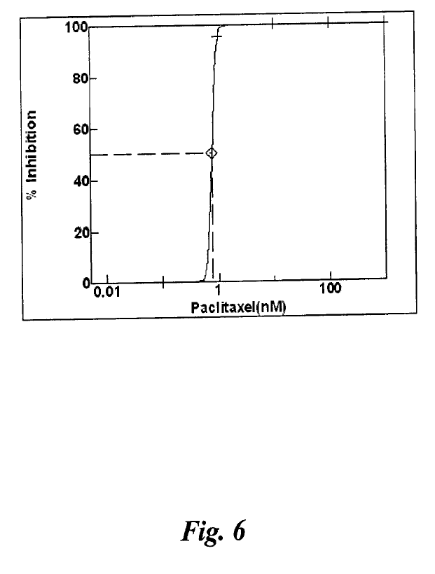

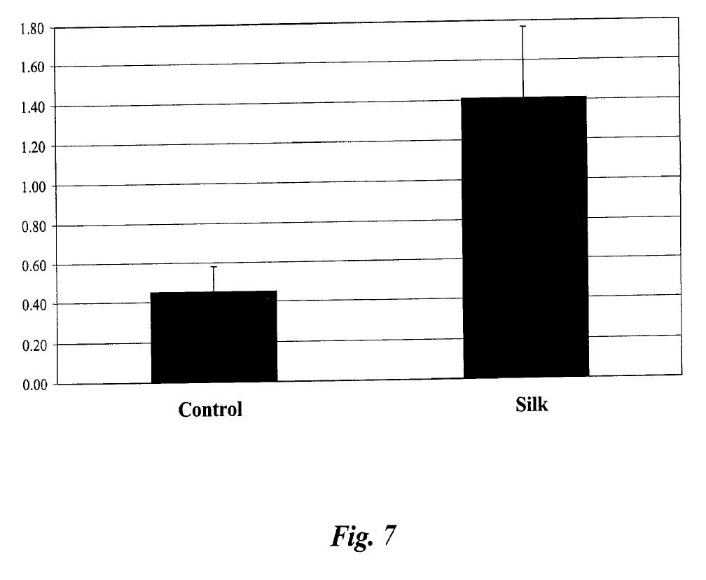

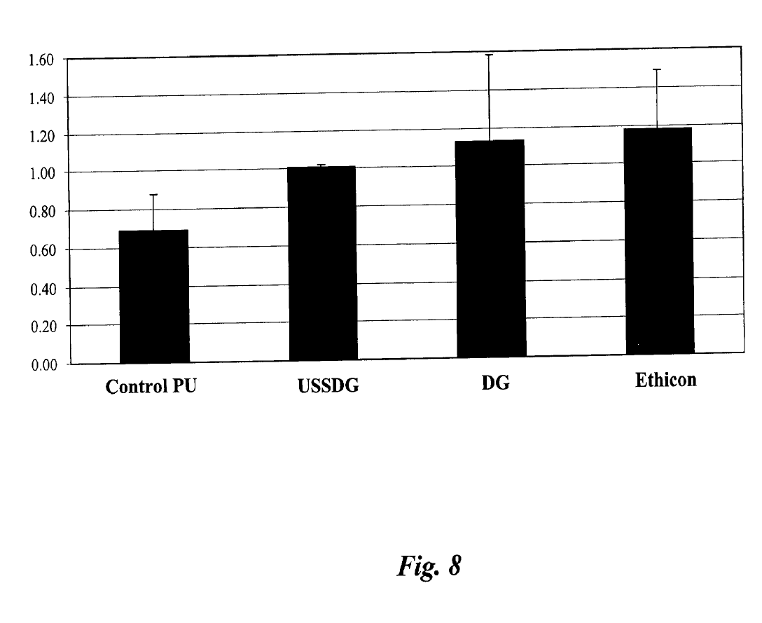

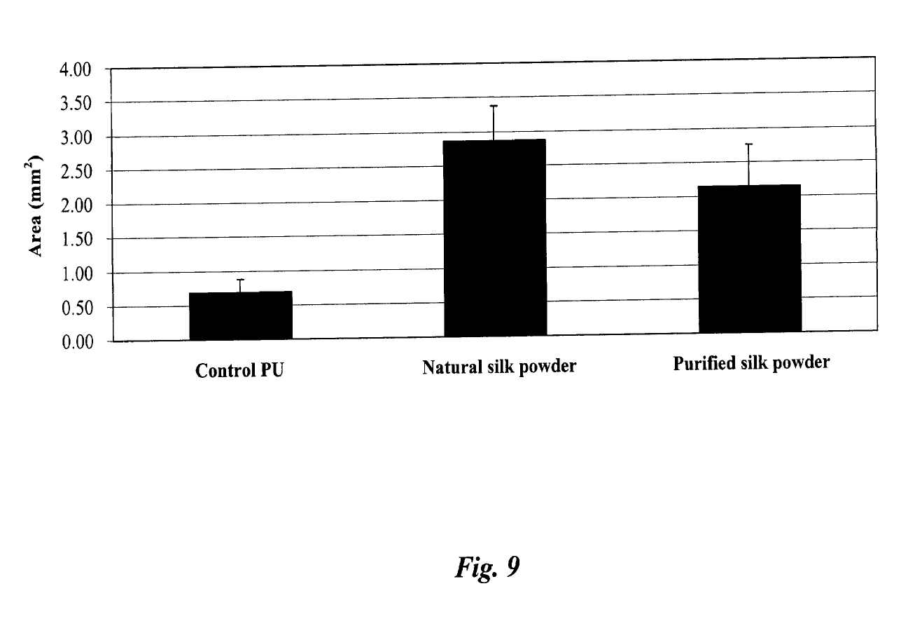

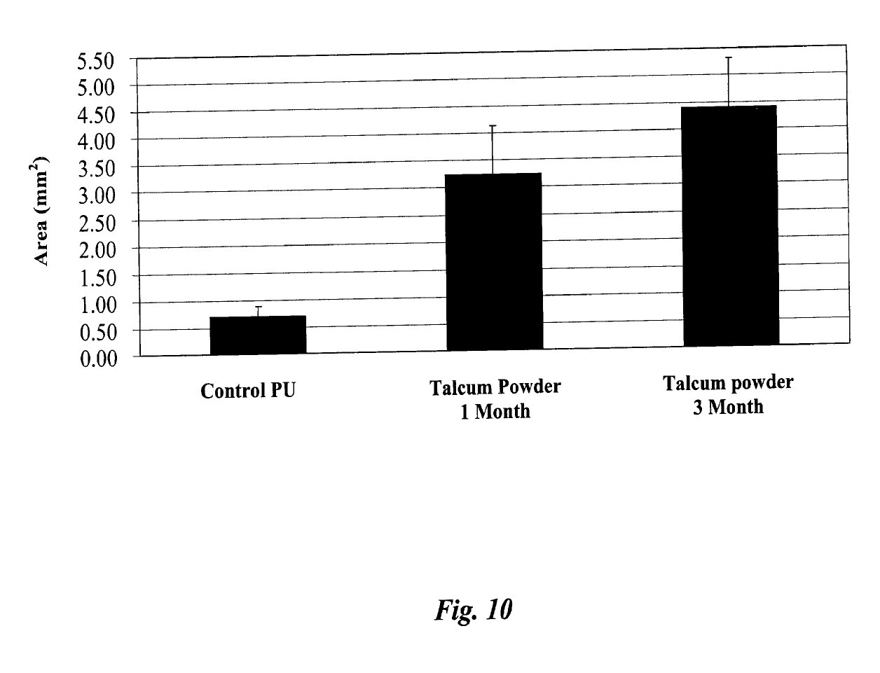

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. No. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl] (4-armed thiol PEG) and pentaerythritol poly(ethylene glycol)ether tetra-succinimidyl glutarate] (4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (I-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers which contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS3), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The above-referenced polymers are commercially available from Pierce (Rockford, Ill.), under catalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis(2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:

Polymer-Xₘ+Polymer-Yₙ→Polymer-Z-Polymer

wherein m≦2, n≦2, and m+n≦5;

where exemplary X groups include —NH₂, —SH, —OH, —PH₂, CO—NH—NH₂, etc., where the X groups may be the same or different in polymer-Xₘ;

where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and

where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂O)ₙ— or —(CH(CH₃)CH₂O)ₙ— or —(CH₂—CH₂—O)ₙ—(CH(CH₃)CH₂—O)ₙ—. In these cases the synthetic polymer would be difunctional.

The required functional group X or Y is commonly coupled to the polymer backbone by a linking group (represented below as “Q”), many of which are known or possible. There are many ways to prepare the various functionalized polymers, some of which are listed below:

Polymer-Q₁-X+Polymer-Q₂-Y→Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ₙ—; and —O—R₂—CO—NH—, which provide synthetic polymers of the partial structures: polymer-O—(CH₂)ₙ—(X or Y); polymer-S-(CH₂), —(X Or Y); polymer-NH—(CH₂)ₙ—(X or Y); polymer-O₂C—NH—(CH₂)ₙ—(X or Y); polymer-O₂C—(CH₂), —(X or Y); polymer-O₂C—(CR¹H)ₙ—(X or Y); and polymer-O—R₂—CO—NH—(X or Y), respectively. In these structures, n=1-10, R¹═H or alkyl (i.e., CH₃, C₂H₅, etc.); R²═CH₂, or CO—NH—CH₂CH₂; and Q₁ and Q₂ may be the same or different.

For example, when Q₂═OCH₂CH₂ (there is no Q₁ in this case); Y=—CO₂—N(COCH₂)₂; and X═—NH₂, —SH, or —OH, the resulting reactions and Z groups would be as follows:

Polymer-NH₂+Polymer-O—CH₂—CH₂—CO₂—N(COCH₂)₂→Polymer-NH—CO—CH₂—CH₂—O-Polymer;

Polymer-SH+Polymer-O—CH₂—CH₂—CO₂—N(COCH₂)₂→Polymer-S—COCH₂CH₂—O-Polymer; and

Polymer-OH+Polymer-O—CH₂—CH₂—CO₂—N(COCH₂)₂→Polymer-O—COCH₂CH₂—O-Polymer.

An additional group, represented below as “D” can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.

Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), ε-caprolactone, and amino acids. The polymers may be referred to as polylactide, polyglycolide, poly(co-lactide-glycolide); poly-ε-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) would contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis which typically occurs upon exposure of such electrophilic groups to aqueous media. Processes for preparing synthetic hydrophilic polymers containing multiple electrophylic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In one aspect, a composition comprising naturally-occurring protein and both of the first and second synthetic polymer as described above is used to form the crosslinked matrix according to the present invention. In one aspect, a composition comprising collagen and both of the first and second synthetic polymer as described above is used to form the crosslinked matrix according to the present invention. In one aspect, a composition comprising methylated collagen and both of the first and second synthetic polymer as described above is used to form the crosslinked matrix according to the present invention. In one aspect, a composition comprising fibrinogen and both of the first and second synthetic polymer as described above is used to form the crosslinked matrix according to the present invention. In one aspect, a composition comprising thrombin and both of the first and second synthetic polymer as described above is used to form the crosslinked matrix according to the present invention. In one aspect, a composition comprising albumin and both of the first and second synthetic polymer as described above is used to form the crosslinked matrix according to the present invention. In one aspect, a composition comprising fibrin and both of the first and second synthetic polymer as described above is used to form the crosslinked matrix according to the present invention. In one aspect, a composition comprising naturally occurring polysaccharide and both of the first and second synthetic polymer as described above is used to form the crosslinked matrix according to the present invention. In one aspect, a composition comprising glycosaminoglycan and both of the first and second synthetic polymer as described above is used to form the crosslinked matrix according to the present invention. In one aspect, a composition comprising deacetylated glycosaminoglycan and both of the first and second synthetic polymer as described above is used to form the crosslinked matrix according to the present invention. In one aspect, a composition comprising desulfated glycosaminoglycan and both of the first and second synthetic polymer as described above is used to form the crosslinked matrix according to the present invention.

In one aspect, a composition comprising naturally-occurring protein and the first synthetic polymer as described above is used to form the crosslinked matrix according to the present invention. In one aspect, a composition comprising collagen and the first synthetic polymer as described above is used to form the crosslinked matrix according to the present invention. In one aspect, a composition comprising methylated collagen and the first synthetic polymer as described above is used to form the crosslinked matrix according to the present invention. In one aspect, a composition comprising fibrinogen and the first synthetic polymer as described above is used to form the crosslinked matrix according to the present invention. In one aspect, a composition comprising thrombin and the first synthetic polymer as described above is used to form the crosslinked matrix according to the present invention. In one aspect, a composition comprising albumin and the first synthetic polymer as described above is used to form the crosslinked matrix according to the present invention. In one aspect, a composition comprising fibrin and the first synthetic polymer as described above is used to form the crosslinked matrix according to the present invention. In one aspect, a composition comprising naturally occurring polysaccharide and the first synthetic polymer as described above is used to form the crosslinked matrix according to the present invention. In one aspect, a composition comprising glycosaminoglycan and the first synthetic polymer as described above is used to form the crosslinked matrix according to the present invention. In one aspect, a composition comprising deacetylated glycosaminoglycan and the first synthetic polymer as described above is used to form the crosslinked matrix according to the present invention. In one aspect, a composition comprising desulfated glycosaminoglycan and the first synthetic polymer as described above is used to form the crosslinked matrix according to the present invention.

In one aspect, a composition comprising naturally-occurring protein and the second synthetic polymer as described above is used to form the crosslinked matrix according to the present invention. In one aspect, a composition comprising collagen and the second synthetic polymer as described above is used to form the crosslinked matrix according to the present invention. In one aspect, a composition comprising methylated collagen and the second synthetic polymer as described above is used to form the crosslinked matrix according to the present invention. In one aspect, a composition comprising fibrinogen and the second synthetic polymer as described above is used to form the crosslinked matrix according to the present invention. In one aspect, a composition comprising thrombin and the second synthetic polymer as described above is used to form the crosslinked matrix according to the present invention. In one aspect, a composition comprising albumin and the second synthetic polymer as described above is used to form the crosslinked matrix according to the present invention. In one aspect, a composition comprising fibrin and the second synthetic polymer as described above is used to form the crosslinked matrix according to the present invention. In one aspect, a composition comprising naturally occurring polysaccharide and the second synthetic polymer as described above is used to form the crosslinked matrix according to the present invention. In one aspect, a composition comprising glycosaminogly can and the second synthetic polymer as described above is used to form the crosslinked matrix according to the present invention. In one aspect, a composition comprising deacetylated glycosaminoglycan and the second synthetic polymer as described above is used to form the crosslinked matrix according to the present invention. In one aspect, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In one aspect, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In one aspect, the polymer may be collagen or a collagen derivative, for example methylated collagen. An example of an in situ forming composition uses pentaerythritol poly(ethylene glycol)ether tetra-sulfhydryl] (4-armed thiol PEG), pentaerythritol poly(ethylene glycol)ether tetra-succinimidyl glutarate] (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

### Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage which commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing soft tissue augmentation in delicate facial tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

### Hydrophilic Polymer+Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae

R¹(-[Q¹]_{q}-X)ₘ (component A), (I)

R²(-[Q²]ᵣ-Y)ₙ (component B), and (II)

R³(-[Q³]ₛ-Fn)ₚ (optional component C), (III)

wherein:

R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;

X represents one of the m nucleophilic groups of component A, and the various X moieties on A may be the same or different;

Y represents one of the n electrophilic groups of component B, and the various Y moieties on A may be the same or different;

Fn represents a functional group on optional component C;

Q¹, Q² and Q³ are linking groups;

m≧2, n≧2, m+n is ≧4, q, and r are independently zero or 1, and when optional component C is present, p≧1, and s is independently zero or 1.

Reactive Groups:

X may be virtually any nucleophilic group, so long as reaction can occur with the electrophilic group Y Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups are particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE

| REPRESENTATIVE | | |
|---|---|---|
| NUCLEOPHILIC | | |
| COMPONENT | REPRESENTATIVE | |
| (A, optional | ELECTROPHILIC | |
| component C | COMPONENT | |
| element FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate terminus) | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂—N(COCH₂) | R¹—NH—(CO)—(CH₂)₃—(CO)—OR² |
| | (succinimidyl glutarate terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹—NH₂ | R²—O—CH₂—CO₂—N(COCH₂) | R¹—NH—(CO)—CH₂—OR² |
| | (succinimidyl acetate terminus) | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCH₂) | R¹—NH—(CO)—(CH₂)₂—(CO)—NH—OR² |
| | (succinimidyl succinamide terminus) | |
| R¹—SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 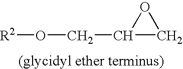 | R¹—NH—CH₂—CH(OH)—CH₂—OR² and R¹—N[CH₂—CH(OH)—CH₂OR²]₂ |
| R¹—NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹—S—CH₂CH₂—SO₂—R² |

Linking Groups:

The functional groups X and Y and FN on optional component C may be directly attached to the compound core (R¹, R² or R³ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages; ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in the following Table:

TABLE

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹-[Q¹]_{q}-X)ₘ, for component A, (II) R²(-[Q²]ᵣ-Y)ₙ for component B, and (III)

R³(-[Q³]ₛ-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components

In one aspect, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6:315), peptide chemistry (see Mutter et al., The Peptides, Academic: New York, N.Y. 2:285-332; and Zalipsky et al., Int. J. Peptide Protein Res. (1987) 30:740), and the synthesis of polymeric drugs (see Zalipsky et al., Eur. Polym. J. (1983) 19:1177; and Ouchi et al., J. Macromol Sci. Chem. (1987) A24:1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Alabama (1997-1998).

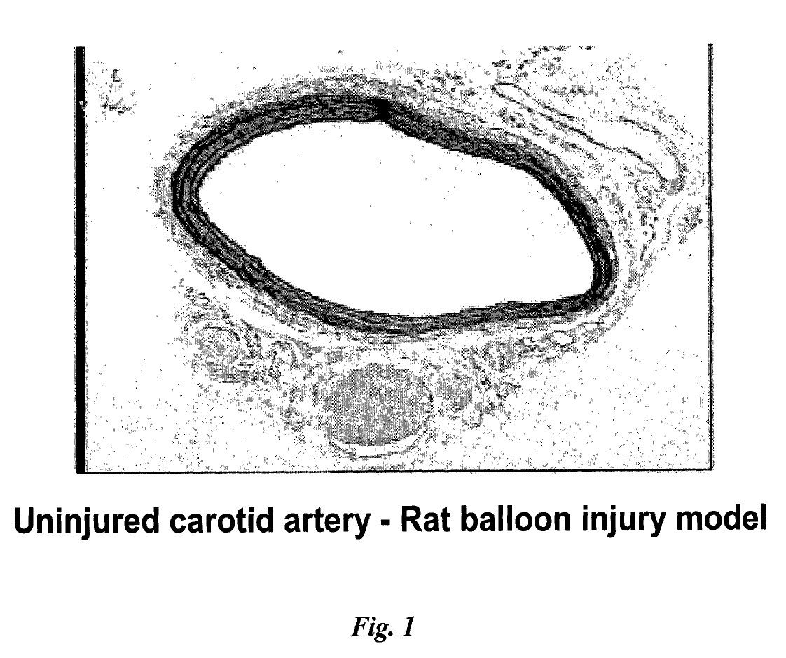

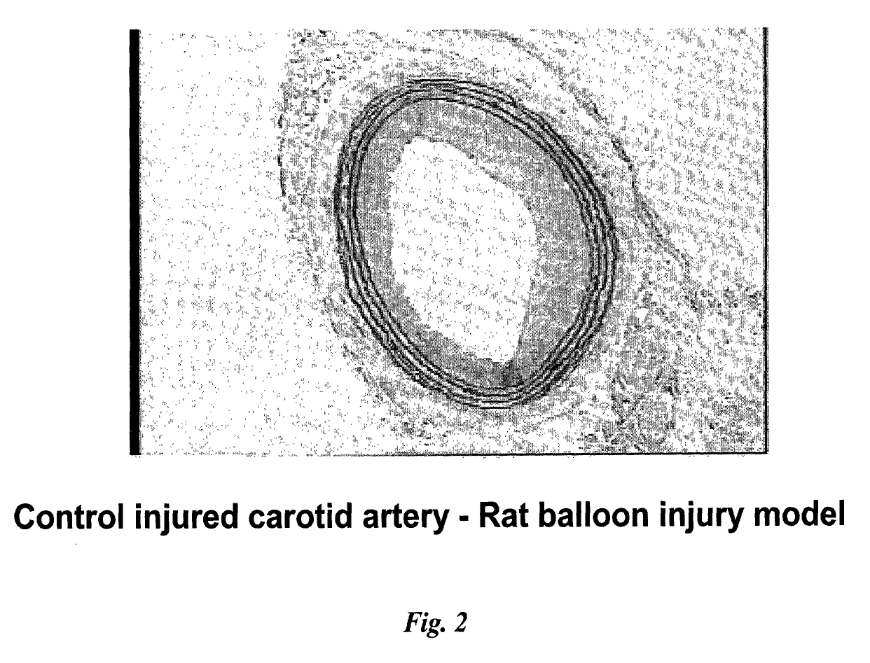

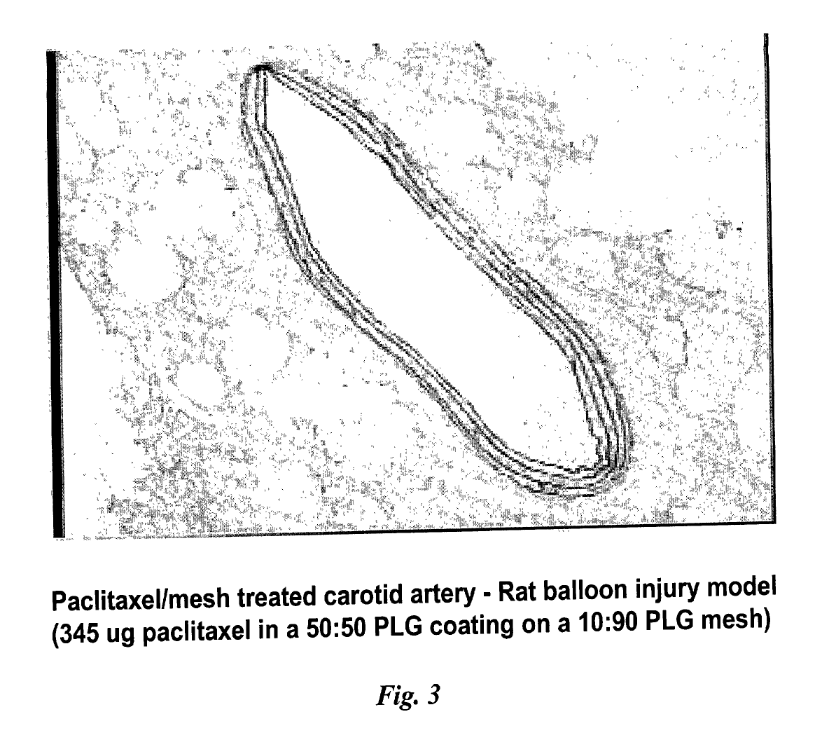

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. Nos. 6,454,786 to Holm et al.; 6,461,325 to Delmotte et al.; 5,585,007 to Antanavich et al.; 5,116,315 to Capozzi et al.; and 4,631,055 to RedI et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen+Fibrinogen and/or Thrombin (e.g., Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient for which the composition is to be delivered. The plasma can be used “as is” after standard preparation which includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NIHu, most usually about 200 to about 500 NIHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NIHu to 10,000 NIHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Pat. Nos. RE38158; 4,839,345; 5,514,379, 5,583,114; 6,458,147; 6,371,975; 5,290,552; 6,096,309; U.S. Patent Application Publication Nos. 2002/0161399; 2001/0018598 and PCT Publication Nos. WO 03/090683; WO 01/45761; WO 99/66964 and WO 96/03159).

### Self-Reactive Compounds

In one aspect, the therapeutic agent is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

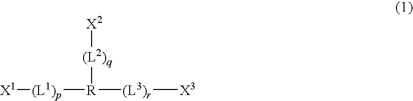

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl which is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):

[X′-(L⁴)ₐ-Y′-(L⁵)_{b}]_{c}—R′

where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II):

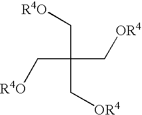

where R⁴ has the formula:

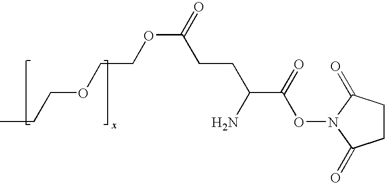

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

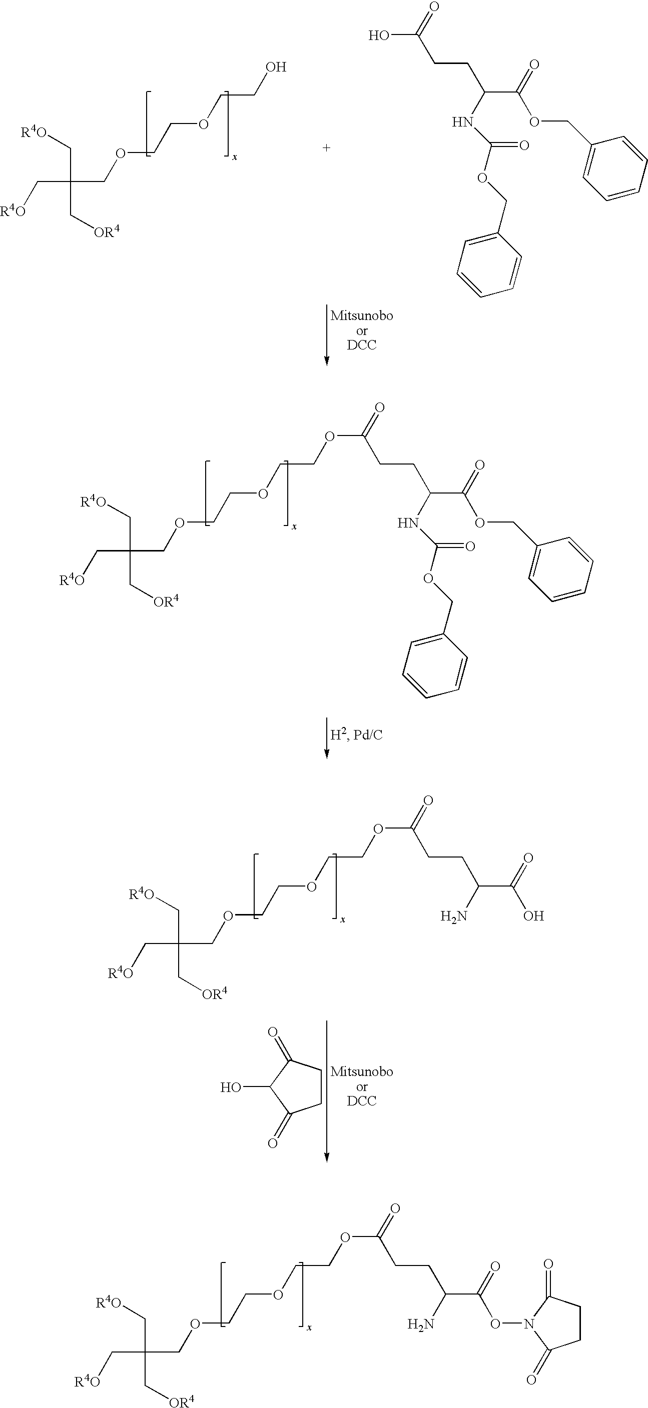

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups is approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

Electrophilic and Nucleophilic Reactive Groups

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (Z_{EL}). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂,

—PHR¹, —P(R¹)₂, —NH—NH₂, —CO—NH—NH₂, —C₅H₄N, etc. wherein R¹ is a hydrocarbyl group and each R¹ may be the same or different. R¹ is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═NH, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C═O); (7) thioisocyanato group (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH₂), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table:

TABLE

| Representative | | |
|---|---|---|
| Nucleophilic | Representative Electrophilic | |
| Group (X, Z_{NU}) | Group (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH₂—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)—(CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S—(CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂—N(COCH₂)₂ | —NH—(CO)—(CH₂)₂—(CO)—NH—O— |
| | succinimidyl succinamide terminus | |
| —SH | —O—NH(CO)—(CH₂)₂—CO₂—N(COCH₂)₂ | —S—(CO)—(CH₂)₂—(CO)—NH—O— |
| —OH | —O—NH(CO)—(CH₂)₂—CO₂—N(COCH₂)₂ | —O—(CO)—(CH₂)₂—(CO)—NH—O— |
| —NH₂ | —O—(CH₂)₂—CHO | —NH—(CO)—(CH₂)₂—O— |
| | propionaldehyde terminus | |
| —NH₂ | 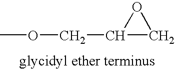 | —NH—CH₂CH(OH)—CH₂—O— and —N[CH₂—CH(OH)—CH₂—O—]₂ |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| | vinyl sulfone terminus | |
| —SH | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH 9.5.

In another embodiment the modification of a dry initial environment comprises dissolving the self-reactive compound in a first buffer solution having a pH within the range of about 1.0 to 5.5 to form a homogeneous solution, and (ii) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

Redox Reactive Groups

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

Oxidative Coupling Reactive Groups

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

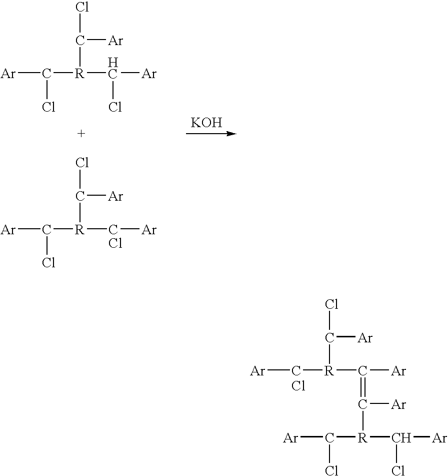

For self-reactive compounds containing oxidative coupling reactive groups, the initial environment typically can be can be dry and sterile, or a non-basic medium. The modification of the initial environment will typically comprise the addition of a base.

Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

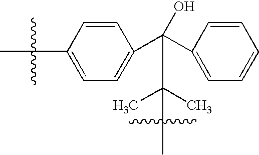

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

Temperature-sensitive Reactive Groups

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

Linking Groups

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; α-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance.

By way of example, particular linking groups and corresponding formulas are indicated in the following Table:

TABLE

| Linking group | Component structure |
|---|---|
| —O—(CH₂)ₓ— | —O—(CH₂)ₓ—X |
| | —O—(CH₂)ₓ—Y |
| | —O—(CH₂)ₓ—Z |
| —S—(CH₂)ₓ— | —S—(CH₂)ₓ—X |
| | —S—(CH₂)ₓ—Y |
| | —S—(CH₂)ₓ—Z |
| —NH—(CH₂)ₓ— | —NH—(CH₂)ₓ—X |
| | —NH—(CH₂)ₓ—Y |
| | —NH—(CH₂)ₓ—Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ—Z |
| —NH—(CO)—O—(CH₂)ₓ— | —NH—(CO)—O—(CH₂)ₓ—X |
| | —NH—(CO)—O—(CH₂)ₓ—Y |
| | —NH—(CO)—O—(CH₂)ₓ—Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ—Z |
| —(CO)—O—(CH₂)ₓ— | —(CO)—O—(CH₂)ₙ—X |
| | —(CO)—O—(CH₂)ₙ—Y |
| | —(CO)—O—(CH₂)ₙ—Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ—Z |
| —O—(CO)—CHR²— | —O—(CO)—CHR²—X |
| | —O—(CO)—CHR²—Y |
| | —O—(CO)—CHR²—Z |
| —O—R³—(CO)—NH— | —O—R³—(CO)—NH—X |
| | —O—R³—(CO)—NH—Y |
| | —O—R³—(CO)—NH—Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Core

The “core” of each self-reactive compound is comprised of the molecular structure to which the reactive groups are bound. The molecular core can a polymer, which includes synthetic polymers and naturally occurring polymers. In one embodiment, the core is a polymer containing repeating monomer units The polymers can be hydrophilic, hydrophobic, or amphiphilic. The molecular core can also be a low molecular weight components such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

Hydrophilic Polymers

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid, poly(acrylamides) such as polyacrylamide per se, poly(methacrylamide), poly(dimethylacrylamide), poly(N-isopropyl-acrylamide), and copolymers thereof; poly(olefinic alcohols) such as poly(vinyl alcohols) and copolymers thereof; poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows. Non-transgenic, recombinant collagen expression in yeast and other cell lines) is described in U.S. Pat. Nos. 6,413,742 to Olsen et al., 6,428,978 to Olsen et al., and 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

Hydrophobic Polymers

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

Amphiphilic Polymers

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains), or a hydrophobic polymer that has been modified with hydrophilic moieties (e.g., “PEGylated” phospholipids such as polyethylene glycolated phospholipids).

Low Molecular Weight Components

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)-based tetracarboxylic acid, heptanedioic acid, octanedioic acid (suberic acid), and hexadecanedioic acid (thapsic acid).

Low molecular weight di- and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

Preparation

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al (1990) Crit. Rev. Therap. Drug Carrier Syst. 6:315), peptide chemistry (see Mutter et al., The Peptides, Academic: New York, N.Y. 2:285-332; and Zalipsky et al. (1987) Int. J. Peptide Protein Res. 30:740), and the synthesis of polymeric drugs (see Zalipsky et al. (1983) Eur. Polym. J. 19:1177; and Ouchi et al. (1987) J. Macromol Sci Chem. A24:1011).

Functionalized forms of PEG, including multi-functionalized PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, Calif., formerly Shearwater Polymers, Huntsville, Ala.) and from Huntsman's Performance Chemicals Group (Houston, Tex.) under the name Jeffamine® polyoxyalkyleneamines-Multi-amino PEGs useful in the present invention include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol) ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

### Other In Situ Crosslinking Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., U.S. Pat. No. 6,632,457). The in situ forming material may be a multi-component hydrogel system (see, e.g., U.S. Pat. No. 6,379,373). The in situ forming material may be a multi-arm block copolymer that includes a central core molecule, such as a residue of a polyol, and at least three copolymer arms covalently attached to the central core molecule, each copolymer arm comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water soluble macromers (see, e.g., U.S. Pat. No. 5,410,016). The in situ forming material may be formed from a two component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene) glycol polymer solution whereby upon mixing the solution cross-links to form a mechanical non-liquid covering structure which acts as a sealant. See e.g., U.S. Pat. Nos. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol) See e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See e.g., U.S. Pat. No. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See e.g., U.S. Patent Application No. 2004/0063613A1 and PCT Publication Nos. WO 01/45761 and WO 03/090683 The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See e.g., U.S. Patent Application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See e.g., PCT Publication No. WO 99/66964. The in situ forming material may be composed of collagen and a peroxidase and hydrogen peroxide, such that the collagen is crosslinked to from a semi-solid gel that seals a wound. See e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See e.g., U.S. Pat. No. 5,206,331.

In another embodiment, the reagents can undergo an electrophilic-nucleophilic reaction to produce a crosslinked matrix. Polymers containing and/or terminated with nucleophilic groups such as amine, sulfhydryl, hydroxyl, —PH₂ or CO—NH—NH₂ can be used as the nucleophilic reagents and polymers containing and/or terminated with electrophilic groups such as succinimidyl, carboxylic acid, aldehyde, epoxide, isocyanate, vinyl, vinyl sulfone, maleimide, —S—S—(C₅H₄N) or activated esters, such as are used in peptide synthesis can be used as the electrophilic reagents. For example, a 4-armed thiol derivatized poly(ethylene glycol) (e.g., pentaerythritol poly(ethylene glycol)ether tetra-sulfhydryl) can be reacted with a 4 armed NHS-derivatized polyethylene glycol (e.g., pentaerythritol poly(ethylene glycol)ether tetra-succinimidyl glutarate) under basic conditions (pH >about 8). Representative examples of compositions that undergo such electrophilic-nucleophilic crosslinking reactions are described, for example, in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; and PCT Application Publication Nos. WO 04/060405 and WO 04/060346. Other examples of in situ forming materials that can be used include those based on the crosslinking of proteins (described in U.S. Pat. Nos. RE38158; 4,839,345; 5,514,379, 5,583,114; 6,458,147; 6,371,975; U.S. Patent Application Publication Nos. 2002/0161399; 2001/0018598 and PCT Publication Nos. WO 03/090683; WO 01/45761; WO 99/66964 and WO 96/03159).

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl) (4-armed thiol PEG), pentaerythritol poly(ethylene glycol)ether tetra-succinimidyl glutarate) (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another embodiment, the reagents that can form a covalent bond with the tissue to which it is applied may be used. Polymers containing and/or terminated with electrophilic groups such as succinimidyl, aldehyde, epoxide, isocyanate, vinyl, vinyl sulfone, maleimide, —S—S—(C₅H₄N) or activated esters, such as are used in peptide synthesis may be used as the reagents. For example, a 4 armed NHS-derivatized polyethylene glycol (e.g., pentaerythritol poly(ethylene glycol)ether tetra-succinimidyl glutarate) may be applied to the tissue in the solid form or in a solution form. In the preferred embodiment, the 4 armed NHS-derivatized polyethylene glycol is applied to the tissue under basic conditions (pH >about 8). Other representative examples of compositions of this nature that may be used are disclosed in PCT Application Publication No. WO 04/060405 and WO 04/060346, and U.S. patent application Ser. No. 10/749,123.

In another embodiment, the in situ forming material polymer can be a polyester. Polyesters that can be used in in situ forming compositions include poly(hydroxyesters). In another embodiment, the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one. Representative examples of these types of compositions are described in U.S. Pat. Nos 5,874,500; 5,936,035; 6,312,725; 6,495,127 and PCT Publication Nos. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine, etc.).

Other examples of in situ forming materials that can be used include those based on the crosslinking of proteins (described in, for example, U.S. Pat. Nos. RE38158; 4,839,345; 5,514,379, 5,583,114; 6,310,036; 6,458,147; 6,371,975; US Patent Application Publication Nos. 2004/0063613A1, 2002/0161399A1, and 2001/0018598A1, and PCT Publication Nos. WO 03/090683, WO 01/45761, WO 99/66964, and WO 96/03159) and those based on isocyanate or isothiocyanate capped polymers (see, e.g., PCT Publication No. WO 04/021983).

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate), and poly(octylcyanoacrylate)).

Examples of commercially available cyanoacrylates that can be used in the present invention include DERMABOND, INDERMIL, GLUSTITCH, VETBOND, HISTOACRYL, TISSUMEND, HISTOACRYL BLUE and ORABASE SOOTHE-N-SEAL LIQUID PROTECTANT.

In another embodiment, the cyanoacrylate compositions may further comprise additives to stabilize the reagents and/or alter the rate of reaction of the cyanoacrylate, and/or plasticize the poly(cyanoacrylate), and/or alter the rate of degradation of the poly(cyanoacrylate). For example, a trimethylene carbonate based polymer or an oxalate polymer of poly(ethylene glycol) or a ε-caprolactone based copolymer may be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICS group of polymers including but not limited to PLURONIC F127 or F68). Representative examples of these compositions are described in U.S. Pat. No. 6,699,940.

Within another aspect of the invention, the biologically active ant-infective and/or fibrosis-inhibiting agent can be delivered with a non-polymeric compound (e.g., a carrier). These non-polymeric carriers can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydroxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Within certain embodiments of the invention, the therapeutic compositions are provided that include (i) a fibrosis-inhibiting agent and/or (ii) an anti-infective agent. The therapeutic compositions may include one or more additional therapeutic agents (such as described above), for example, anti-inflammatory agents, anti-thrombotic agents, and/or anti-platelet agents. Other agents that may be combined with the therapeutic compositions include, e.g., additional ingredients such as surfactants (e.g., PLURONICS, such as F-127, L-122, L-101, L-92, L-81, and L-61), preservatives, anti-oxidants.

In one aspect, the present invention provides compositions comprising i) an anti-fibrotic agent and ii) a polymer or a compound that forms a polymer in situ. The following are some, but by no means all, of the preferred anti-fibrotic agents and classes of anti-fibrotic agents that may be included in the inventive compositions:

1a) an anti-fibrotic agent that inhibits cell regeneration,

2a) an anti-fibrotic agent that inhibits angiogenesis,

3a) an anti-fibrotic agent that inhibits fibroblast migration,

4a) an anti-fibrotic agent that inhibits fibroblast proliferation,

5a) an anti-fibrotic agent that inhibits deposition of extracellular matrix,

6a) an anti-fibrotic agent inhibits tissue remodeling,

7a) an adensosine A2A receptor antagonist,

8a) an AKT inhibitor,

9a) an alpha 2 integrin antagonist, wherein the alpha 2 integrin antagonist is Pharmaprojects No. 5754 (Merck KgaA),

10a) an alpha 4 integrin antagonist,

11a) an alpha 7 nicotinic receptor agonist,

12a) an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Pharminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Pharminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof,

13a) an apoptosis antagonist,

14a) an apoptosis activator,

15a) a beta 1 integrin antagonist,

16a) a beta tubulin inhibitor,

17a) a blocker of enzyme production in Hepatitis C,

18a) a Bruton's tyrosine kinase inhibitor,

19a) a calcineurin inhibitor,

20a) a caspase 3 inhibitor,

21a) a CC chemokine receptor antagonist,

22a) a cell cycle inhibitor selected from the group consisting of SNS-595 (Sunesis), synthadotin, KRX-0403, homoharringtonine, and an analogue or derivative thereof,

23a) a cathepsin B inhibitor,

24a) a cathepsin K inhibitor, wherein the cathepsin K inhibitor is 462795 (GlaxoSmithKline), INPL-022-D6 (Amura Therapeutics), or an analogue or derivative thereof,

25a) a cathepsin L inhibitor,

26a) a CD40 antagonist,

27a) a chemokine receptor agonist,

28a) a chymase inhibitor,

29a) a collagenase antagonist,

30a) a CXCR antagonist,

31a) a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), CGP 74514A, bohemine, olomoucine (CAS No. 101622-51-9), indole-3-carbinol (CAS No. 700-06-1), and an analogue or derivative thereof,

32a) a cyclooxygenase 1 inhibitor,

33a) a DHFR inhibitor,

34a) a dual integrin inhibitor,

35a) an elastase inhibitor,

36a) an elongation factor-1 alpha inhibitor,

37a) an endothelial growth factor antagonist,

38a) an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), lavendustin A (CAS No. 125697-92-9), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), SU 1498 (a VEGF-R inhibitor), a VEGFR-2 kinase inhibitor (Bristol-Myers Squibb), XL-647 (Exelixis), a KDR inhibitor from Abbott Laboratories, sorafenib tosylate, and an analogue or derivative thereof,

39a) an endotoxin antagonist,

40a) an epothilone and tubulin binder,

41a) an estrogen receptor antagonist,

42a) an FGF inhibitor,

43a) a farnexyl transferase inhibitor,

44a) a farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof,

45a) an FLT-3 kinase inhibitor,

46a) an FGF receptor kinase inhibitor,

47a) a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), mevastatin, and an analogue or derivative thereof,

48a) a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), radicicol from Humicola fuscoatra (CAS No. 12772-57-5), and an analogue or derivative thereof,

49a) a histone deacetylase inhibitor,

50a) an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), cerivastatin Na (CAS No. 143201-11-0), and an analogue or derivative thereof,

51a) an ICAM inhibitor,

52a) an IL, ICE and IRAK antagonist, wherein the antagonist is a CJ-14877, CP-424174 (Pfizer), NF-61 (Negma-Lerads), and an analogue or derivative thereof,

53a) an IL-2 inhibitor,

54a) an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof,

55a) an IMPDH (inosine monophosphate),

56a) an integrin antagonist,

57a) an interleukin antagonist,

58a) an inhibitor of type III receptor tyrosine kinase,

59a) an irreversible inhibitor of enzyme methionine aminopeptidase type 2,

60a) an isozyme selective delta protein kinase C inhibitor,

61a) a JAK3 enzyme inhibitor,

62a) a JNK inhibitor,

63a) a kinase inhibitor,

64a) a kinesin antagonist,

65a) a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof,

66a) a MAP kinase inhibitor,

67a) a matrix metalloproteinase inhibitor,

68a) an MCP-CCR2 inhibitor,

69a) an mTOR inhibitor,

70a) an mTOR kinase inhibitor,

71a) a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, dolastatin 15 (CAS No. 123884-00-4), vincamine, and an analogue or derivative thereof,

72a) an MIF inhibitor,

73a) an MMP inhibitor,

74a) a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 13916747-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof,

75a) an NF kappa B inhibitor selected from the group consisting of emodin (CAS No. 518-82-1), AVE-0545 or AVE-0547 (Sanofi-Aventis), bortezomib (CAS No. 179324-69-7) (Millennium Pharmaceuticals), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), Bay 11-7085, and an analogue or derivative thereof,

76a) a nitric oxide agonist,

77a) an ornithine decarboxylase inhibitor,

78a) a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), SKF86002 (CAS No. 72873-74-6), RPR-200765A (Sanofi-Aventis), SD-282 (Johnson & Johnson), TAK-715 (Takeda), and an analogue or derivative thereof,

79a) a palmitoyl-protein thioesterase inhibitor,

80a) a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof,

81a) a peroxisome proliferators-activated receptor agonist selected from the group consisting of (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-154) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-R modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), LBM642, WY-14,643 (CAS No. 50892-23-4), GW7647, fenofibric acid (CAS No. 42017-89-0), MCC-555 (CAS No. 161600-01-7), GW9662, GW1929, GW501516, L-165,041 (CAS No. 79558-09-1), and an analogue or derivative thereof,

82a) a phosphatase inhibitor,

83a) a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), theobromine (CAS No. 83-67-0), papverine hydrochloride (CAS No. 61-25-6), quercetin dehydrate (CAS No. 6151-25-3), YM 976 (CAS No. 191219-80-4), irsogladine (CAS No. 57381-26-7), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof,

84a) a PKC inhibitor,

85a) a platelet activating factor antagonist,

86a) a platelet-derived growth factor receptor kinase inhibitor,

87a) a prolyl hydroxylase inhibitor,

88a) a polymorphonuclear neutrophil inhibitor,

89a) a protein kinase B inhibitor,

90a) a protein kinase C stimulant,

91a) a purine nucleoside analogue,

92a) a purinoreceptor P2X antagonist,

93a) a Raf kinase inhibitor,

94a) a reversible inhibitor of ErbB1 and ErbB2,

95a) a ribonucleoside triphosphate reductase inhibitor,

96a) an SDF-1 antagonist,

97a) a sheddase inhibitor,

98a) an SRC inhibitor,

99a) a stromelysin inhibitor,

100a) an Syk kinase inhibitor,

101a) a telomerase inhibitor,

102a) a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof,

103a) a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-1 38 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No-37339-90-5) (Ajinomoto), MDL-201112 (CAS No-142130-73-2) (Sanofi-Aventis), medroxyprogesterone (CAS No. 520-85-4) (Inkine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No-199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof,

104a) a tumor necrosis factor antagonist,

105a) a Toll receptor inhibitor,

106a) a tubulin antagonist,

107a) a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Nyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), herbimycin A, and an analogue or derivative thereof,

108a) a VEGF inhibitor,

109a) a vitamin D receptor agonist,

110a) ZD-6474 (an angiogenesis inhibitor),

111a) AP-23573 (an mTOR inhibitor),

112a) synthadotin (a tubulin antagonist),

113a) S-0885 (a collagenase inhibitor),

114a) aplidine (an elongation factor-1 alpha inhibitor),

115a) ixabepilone (an epithilone),

116a) IDN-5390 (an angiogenesis inhibitor and an FGF inhibitor),

117a) SB-2723005 (an angiogenesis inhibitor),

118a) ABT-518 (an angiogenesis inhibitor),

119a) combretastatin (an angiogenesis inhibitor),

120a) anecortave acetate (an angiogenesis inhibitor),

121a) SB-715992 (a kinesin antagonist),

122a) temsirolimus (an mTOR inhibitor),

123a) adalimumab (a TNFα antagonist),

124a) erucylphosphocholine (an ATK inhibitor),

125a) alphastatin (an angiogenesis inhibitor),

126a) bortezomib (an NF Kappa B inhibitor),

127a) etanercept (a TNFα antagonist and TACE inhibitor),

128a) humicade (a TNFα inhibitor),

129a) gefitinib (a tyrosine kinase inhibitor),

130a) a histamine receptor antagonist selected from the group consisting of phenothiazines (e.g., promethazine), alkylamines (e.g., chlorpheniramine (CAS No. 7054-11-7), brompheniramine (CAS No. 980-71-2), fexofenadine hydrochloride, promethazine hydrochloride, loratadine, ketotifen fumarate salt, and acrivastine), methylxanthines (e.g., theophylline, theobromine, and caffeine), cimetidine (available under the tradename TAGAMET from SmithKline Beecham Pharmaceutical Co., Wilmington, Del.), ranitidine (available under the tradename ZANTAC from Warner Lambert Company, Morris Plains, N.J.), famotidine (available under the tradename PEPCID from Merck & Co., Whitehouse Station, N.J.), nizatidine (available under the tradename AXID from Reliant Pharmaceuticals, Inc., Liberty Corner, N.J.), nizatidine, and roxatidine acetate (CAS No. 78628-28-1), H3 receptor antagonists (e.g., thioperamide and thioperamide maleate salt), and anti-histamines (e.g., tricyclic dibenozoxepins, ethanolamines, ethylenediamines, piperizines, piperidines, and pthalazinones),

131a) an alpha adrenergic receptor antagonist,

132a) an anti-psychotic compound,

133a) a CaM kinase II inhibitor,

134a) a G protein agonist,

135a) an antibiotic selected from the group consisting of apigenin (Cas No. 520-36-5), ampicillin sodium salt (CAS No. 69-52-3), puromycin, and an analogue or derivative thereof,

136a) an anti-microbial agent,

137a) a DNA topoisomerase inhibitor selected from the group consisting of β-lapachone (CAS No. 4707-32-8), (−)-arctigenin (CAS No. 7770-78-7), aurintricarboxylic acid, and an analogue or derivative thereof,

138a) a thromboxane A2 receptor inhibitor selected from the group consisting of BM-531 (CAS No. 284464-46-6), ozagrel hydrochloride (CAS No. 78712-43-3), and an analogue or derivative thereof,

139a) a D2 dopamine receptor antagonist,

140a) a Peptidyl-Prolyl Cis/Trans Isomerase Inhibitor,

141a) a dopamine antagonist, an anesthetic compound,

142a) a clotting factor,

143a) a lysyl hydrolase inhibitor,

144a) a muscarinic receptor inhibitor,

145a) a superoxide anion generator,

146a) a steroid,

147a) an anti-proliferative agent selected from the group consisting of silibinin (CAS No. 22888-70-6), silymarin (CAS No. 65666-07-1), 1,2-hexanediol, dioctyl phthalate (CAS No. 117-81-7), zirconium (IV) oxide, glycyrrhizic acid, spermidine trihydrochloride, tetrahydrochloride, CGP 74514, spermine tetrahydrochloride, NG-methyl-L-arginine acetate salt, galardin, and an analogue or derivative thereof,

148a) a diuretic,

149a) an anti-coagulant,

150a) a cyclic GMP agonist,

151a) an adenylate cyclase agonist,

152a) an antioxidant,

153a) a nitric oxide synthase inhibitor,

154a) an anti-neoplastic agent selected from tirapazamine (CAS No. 27314-97-2), fludarabine (CAS No. 21679-14-1), cladribine, imatinib mesilate, and an analogue or derivative thereof,

155a) a DNA synthesis inhibitor,

156a) a DNA alkylating agent selected from dacarbazine (CAS No. 4342-03-4), temozolomide, procarbazine HCl, and an analogue or derivative thereof,

157a) a DNA methylation inhibitor,

158a) a NSAID agent,

159a) a peptidylglycine alpha-hydroxylating monooxygenase inhibitor,

160a) an MEK1/MEK 2 inhibitor,

161a) a NO synthase inhibitor,

162a) a retinoic acid receptor antagonist selected from isotretinoin (CAS No. 4759-48-2) and an analogue or derivative thereof,

163a) an ACE inhibitor,

164a) a glycosylation inhibitor,

165a) an intracellular calcium influx inhibitor,

166a) an anti-emetic agent,

167a) an acetylcholinesterase inhibitor,

168a) an ALK-5 receptor antagonist,

169a) a RAR/RXT antagonist,

170a) an eIF-2a inhibitor,

171a) an S-adenosyl-L-homocysteine hydrolase inhibitor,

172a) an estrogen agonist,

173a) a serotonin receptor inhibitor,

174a) an anti-thrombotic agent,

175a) a tryptase inhibitor,

176a) a pesticide,

177a) a bone mineralization promoter,

178a) a bisphosphonate compound selected from risedronate and an analogue or derivative thereof,

179a) an anti-inflammatory compound,

180a) a DNA methylation promoter,

181a) an anti-spasmodic agent,

182a) a protein synthesis inhibitor,

183a) an α-glucosidase inhibitor,

184a) a calcium channel blocker,

185a) a pyruvate dehydrogenase activator,

186a) a prostaglandin inhibitor,

187a) a sodium channel inhibitor,

188a) a serine protease inhibitor,

189a) an intracellular calcium flux inhibitor,

190a) a JAK2 inhibitor;

191a) an androgen inhibitor,

192a) an aromatase inhibitor,

193a) an anti-viral agent,

194a) a 5-HT inhibitor,

195a) an FXR antagonist,

196a) an actin polymerization and stabilization promoter,

197a) an AXOR12 agonist,

198a) an angiotensin II receptor agonist,

199a) a platelet aggregation inhibitor,

200a) a CB1/CB2 receptor agonist,

201a) a norepinephrine reuptake inhibitor,

202a) a selective serotonin reuptake inhibitor,

203a) a reducing agent,

204a) isotretinoin,

205a) radicicol,

206a) clobetasol propionate,

207a) homoharringtonine,

208a) trichostatin A,

209a) brefeldin A,

210a) thapsigargin,

211a) dolastatin 15,

212a) cerivastatin,

213a) jasplakinolide,

214a) herbimycin A,

215a) pirfenidone,

216a) vinorelbine,

217a) 17-DMAG,

218a) tacrolimus,

219a) loteprednol etabonate,

220a) juglone,

221a) prednisolone,

222a) puromycin,

223a) 3-BAABE,

224a) cladribine,

225a) mannose-6-phosphate,

226a) 5-azacytidine,

227a) Ly333531 (ruboxistaurin),

228a) simvastatin, and

229a) an immuno-modulator selected from Bay 11-7085, (−)-arctigenin, idazoxan hydrochloride, and an analogue or derivative thereof.

As mentioned above, the present invention provides compositions comprising each of the foregoing 229 (i.e., 1a through 229a) listed anti-fibrotic agents or classes of anti-fibrotic agents, with each of the following 98 (i.e., 1b through 97b) polymers and compounds:

1b. A crosslinked polymer.

2b. A polymer that reacts with mammalian tissue.

3b. A polymer that is a naturally occurring polymer.

4b. A polymer that is a protein.

5b. A polymer that is a carbohydrate.

6b. A polymer that is biodegradable.

7b. A polymer that is crosslinked and biodegradable.

8b. A polymer that nonbiodegradable.

9b. Collagen.

10b. Methylated collagen.

11b. Fibrinogen.

12b. Thrombin.

13b. Albumin.

14b. Plasminogen.

15b. von Willebrands factor.

16b. Factor VIII.

17b. Hypoallergenic collagen.

18b. Atelopeptidic collagen.

19b. Telopeptide collagen.

20b. Crosslinked collagen.

21b. Aprotinin.

22b. Gelatin.

23b. A protein conjugate.

24b. A gelatin conjugate.

25b. Hyaluronic acid.

26b. A hyaluronic acid derivative.

27b. A synthetic polymer.

28b. A polymer formed from reactants comprising a synthetic isocyanate-containing compound.

29b. A synthetic isocyanate-containing compound.

30b. A polymer formed from reactants comprising a synthetic thiol-containing compound.

31b. A synthetic thiol-containing compound.

32b. A polymer formed from reactants comprising a synthetic compound containing at least two thiol groups.

33b. A synthetic compound containing at least two thiol groups.

34b. A polymer formed from reactants comprising a synthetic compound containing at least three thiol groups.

35b. A synthetic compound containing at least three thiol groups.

36b. A polymer formed from reactants comprising a synthetic compound containing at least four thiol groups.

37b. A synthetic compound containing at least four thiol groups.

38b. A polymer formed from reactants comprising a synthetic amino-containing compound.

39b. A synthetic amino-containing compound.

40b. A polymer formed from reactants comprising a synthetic compound containing at least two amino groups.

41b. A synthetic compound containing at least two amino groups.

42b. A polymer formed from reactants comprising a synthetic compound containing at least three amino groups.

43b. A synthetic compound containing at least three amino groups.

44b. A polymer formed from reactants comprising a synthetic compound containing at least four amino groups.

45b. A synthetic compound containing at least four amino groups.

46b. A polymer formed from reactants comprising a synthetic compound comprising a carbonyl-oxygen-succinimidyl group.

47b. A synthetic compound comprising a carbonyl-oxygen-succinimidyl group.

48b. A polymer formed from reactants comprising a synthetic compound comprising at least two carbonyl-oxygen-succinimidyl groups.

49b. A synthetic compound comprising at least two carbonyl-oxygen-succinimidyl groups.

50b. A polymer formed from reactants comprising a synthetic compound comprising at least three carbonyl-oxygen-succinimidyl groups.

51b. A synthetic compound comprising at least three carbonyl-oxygen-succinimidyl groups.

52b. A polymer formed from reactants comprising a synthetic compound comprising at least four carbonyl-oxygen-succinimidyl groups.

53b. A synthetic compound comprising at least four carbonyl-oxygen-succinimidyl groups.

54b. A polymer formed from reactants comprising a synthetic polyalkylene oxide-containing compound.

55b. A synthetic polyalkylene oxide-containing compound.

56b. A polymer formed from reactants comprising a synthetic compound comprising both polyalkylene oxide and biodegradable polyester blocks.

57b. A synthetic compound comprising both polyalkylene oxide and biodegradable polyester blocks.

58b. A polymer formed from reactants comprising a synthetic polyalkylene oxide-containing compound having reactive amino groups.

59b. A synthetic polyalkylene oxide-containing compound having reactive amino groups.

60b. A polymer formed from reactants comprising a synthetic polyalkylene oxide-containing compound having reactive thiol groups.

61b. A synthetic polyalkylene oxide-containing compound having reactive thiol groups.

62b. A polymer formed from reactants comprising a synthetic polyalkylene oxide-containing compound having reactive carbonyl-oxygen-succinimidyl groups.

63b. A synthetic polyalkylene oxide-containing compound having reactive carbonyl-oxygen-succinimidyl groups.

64b. A polymer formed from reactants comprising a synthetic compound comprising a biodegradable polyester block.

65b. A synthetic compound comprising a biodegradable polyester block.

66b. A polymer formed from reactants comprising a synthetic polymer formed in whole or part from lactic acid or lactide.

67b. A synthetic polymer formed in whole or part from lactic acid or lactide.

68b. A polymer formed from reactants comprising a synthetic polymer formed in whole or part from glycolic acid or glycolide.

69b. A synthetic polymer formed in whole or part from glycolic acid or glycolide.

70b. A polymer formed from reactants comprising polylysine.

71b. Polylysine.

72b. A polymer formed from reactants comprising (a) protein and (b) a compound comprising a polyalkylene oxide portion.

73b. A polymer formed from reactants comprising (a) protein and (b) polylysine.

74b. A polymer formed from reactants comprising (a) protein and (b) a compound having at least four thiol groups.

75b. A polymer formed from reactants comprising (a) protein and (b) a compound having at least four amino groups.

76b. A polymer formed from reactants comprising (a) protein and (b) a compound having at least four carbonyl-oxygen-succinimide groups.

77b. A polymer formed from reactants comprising (a) protein and (b) a compound having a biodegradable region formed from reactants selected from lactic acid, lactide, glycolic acid, glycolide, and epsilon-caprolactone.

78b. A polymer formed from reactants comprising (a) collagen and (b) a compound comprising a polyalkylene oxide portion.

79b. A polymer formed from reactants comprising (a) collagen and (b) polylysine.

80b. A polymer formed from reactants comprising (a) collagen and (b) a compound having at least four thiol groups.

81b. A polymer formed from reactants comprising (a) collagen and (b) a compound having at least four amino groups.

82b. A polymer formed from reactants comprising (a) collagen and (b) a compound having at least four carbonyl-oxygen-succinimide groups.

83b. A polymer formed from reactants comprising (a) collagen and (b) a compound having a biodegradable region formed from reactants selected from lactic acid, lactide, glycolic acid, glycolide, and epsilon-caprolactone.

84b. A polymer formed from reactants comprising (a) methylated collagen and (b) a compound comprising a polyalkylene oxide portion.

85b. A polymer formed from reactants comprising (a) methylated collagen and (b) polylysine.

86b. A polymer formed from reactants comprising (a) methylated collagen and (b) a compound having at least four thiol groups.

87b. A polymer formed from reactants comprising (a) methylated collagen and (b) a compound having at least four amino groups.

88b. A polymer formed from reactants comprising (a) methylated collagen and (b) a compound having at least four carbonyl-oxygen-succinimide groups.

89b. A polymer formed from reactants comprising (a) methylated collagen and (b) a compound having a biodegradable region formed from reactants selected from lactic acid, lactide, glycolic acid, glycolide, and epsilon-caprolactone.

90b. A polymer formed from reactants comprising hyaluronic acid.

91b. A polymer formed from reactants comprising a hyaluronic acid derivative.

92b. A polymer formed from reactants comprising pentaerythritol poly(ethylene glycol)ether tetra-sulfhydryl of number average molecular weight between 3,000 and 30,000.

93b. Pentaerythritol poly(ethylene glycol)ether tetra-sulfhydryl of number average molecular weight between 3,000 and 30,000.

94b. A polymer formed from reactants comprising pentaerythritol poly(ethylene glycol)ether tetra-amino of number average molecular weight between 3,000 and 30,000.

95b. Pentaerythritol poly(ethylene glycol)ether tetra-amino of number average molecular weight between 3,000 and 30,000.

96b. A polymer formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

97b. A mixture of (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

As mentioned above, the present invention provides compositions comprising each of the foregoing 229 (1a through 229a) listed anti-fibrotic agents or classes of anti-fibrotic agents, with each of the foregoing 97 (1b through 97b) polymers and compounds: Thus, in separate aspects, the invention provides 229 times 97=22213 described compositions. In other words, each of the following is a distinct aspect of the present invention: 1a+1b, 2a+1b, 3a+1b, 4a+1b, 5a+1b, 6a+1b, 7a+1b, 8a+1b, 9a+1, 10a+1b, 11a+1b, 12a+1b, 13a+1b, 14a+1b, 15a+1b, 16a+1b, 17a+1b, 18a+1b, 19a+1b, 20a+1b, 21a+1b, 22a+1b, 23a+1b, 24a+1b, 25a+1b, 26a+1b, 27a+1b, 28a+1b, 29a+1b, 30a+1b, 31a+1b, 32a+1b, 33a+1b, 34a+1b, 35a+1b, 36a+1b, 37a+1b, 38a+1b, 39a+1b, 40a+1b, 41a+1b, 42a+1b, 43a+1b, 44a+1b, 45a+1b, 46a+1b, 47a+1b, 48a+1b, 49a+1b, 50a+1b, 51a+1b, 52a+1b, 53a+1b, 54a+1b, 55a+1b, 56a+1b, 57a+1b, 58a+1b, 59a+1b, 60a+1b, 61a+1b, 62a+1b, 63a+1b, 64a+1b, 65a+1b, 66a+1b, 67a+1b, 68a+1b, 69a+1b, 70a+1b, 71a+1b, 72a+1b, 73a+1b, 74a+1b, 75a+1b, 76a+1b, 77a+1b, 78a+1b, 79a+1b, 80a+1b, 81a+1b, 82a+1b, 83a+1b, 84a+1b, 85a+1b, 86a+1b, 87a+1b, 88a+1b, 89a+1b, 90a+1b, 91a+1b, 92a+1b, 93a+1b, 94a+1b, 95a+1b, 96a+1b, 97a+1b, 98a+1b, 99a+1b, 100a+1b, 101a+1b, 102a+1b, 103a+1b, 104a+1b, 105a+1b, 106a+1b, 107a+1b, 108a+1b, 109a+1b, 110a+1b, 111a+1b, 112a+1b, 113a+1b, 114a+1b, 115a+1b, 116a+1b, 117a+1b, 118a+1b, 119a+1b, 120a+1b, 121a+1b, 122a+1b, 123a+1b, 124a+1b, 125a+1b, 126a+1b, 127a+1b, 128a+1b, 129a+1b, 130a+1b, 131a+1b, 132a+1b, 133a+1b, 134a+1b, 135a+1b, 136a+1b, 137a+1b, 138a+1b, 139a+1b, 140a+1b, 141a+1b, 142a+1b, 143a+1b, 144a+1b, 145a+1b, 146a+1b, 147a+1b, 148a+1b, 149a+1b, 150a+1b, 151a+1b, 152a+1b, 153a+1b, 154a+1b, 155a+1b, 156a+1b, 157a+1b, 158a+1b, 159a+1b, 160a+1b, 161a+1b, 162a+1b, 163a+1b, 164a+1b, 165a+1b, 166a+1b, 167a+1b, 168a+1b, 169a+1b, 170a+1b, 171a+1b, 172a+1b, 173a+1b, 174a+1b, 175a+1b, 176a+1b, 177a+1b, 178a+1b, 179a+1b, 180a+1b, 181a+1b, 182a+1b, 183a+1b, 184a+1b, 185a+1b, 186a+1b, 187a+1b, 188a+1b, 189a+1b, 190a+1b, 191a+1b, 192a+1b, 193a+1b, 194a+1b, 95a+1b, 96a+1b, 97a+1b, 98a+1b, 99a+1b, 200a+1b, 201a+1b, 202a+1b, 203a+1b, 204a+1b, 205a+1b, 206a+1b, 207a+1b, 208a+1b, 209a+1b, 210a+1b, 211a+1b, 212a+1b, 213a+1b, 214a+1b, 215a+1b, 216a+1b, 217a+1b, 218a+1b, 219a+1b, 220a+1b, 221a+1b, 222a+1b, 223a+1b, 224a+1b, 225a+1b, 226a+1b, 227a+1b, 228a+1b, 229a+1b, 1a+2b, 2a+2b, 3a+2b, 4a+2b, 5a+2b, 6a+2b, 7a+2b, 8a+2b, 9a+2b, 10a+2b, 11a+2b, 12a+2b, 13a+2b, 14a+2b, 15a+2b, 16a+2b, 17a+2b, 18a+2b, 19a+2b, 20a+2b, 21a+2b, 22a+2b, 23a+2b, 24a+2b, 25a+2b, 26a+2b, 27a+2b, 28a+2b, 29a+2b, 30a+2b, 31a+2b, 32a+2b, 33a+2b, 34a+2b, 35a+2b, 36a+2b, 37a+2b, 38a+2b, 39a+2b, 40a+2b, 41a+2b, 42a+2b, 43a+2b, 44a+2b, 45a+2b, 46a+2b, 47a+2b, 48a+2b, 49a+2b, 50a+2b, 51a+2b, 52a+2b, 53a+2b, 54a+2b, 55a+2b, 56a+2b, 57a+2b, 58a+2b, 59a+2b, 60a+2b, 61a+2b, 62a+2b, 63a+2b, 64a+2b, 65a+2b, 66a+2b, 67a+2b, 68a+2b, 69a+2b, 70a+2b, 71a+2b, 72a+2b, 73a+2b, 74a+2b, 75a+2b, 76a+2b, 77a+2b, 78a+2b, 79a+2b, 80a+2b, 81a+2b, 82a+2b, 83a+2b, 84a+2b, 85a+2b, 86a+2b, 87a+2b, 88a+2b, 89a+2b, 90a+2b, 91a+2b, 92a+2b, 93a+2b, 94a+2b, 95a+2b, 96a+2b, 97a+2b, 98a+2b, 99a+2b, 100a+2b, 101a+2b, 102a+2b, 103a+2b, 104a+2b, 105a+2b, 106a+2b, 107a+2b, 108a+2b, 109a+2b, 110a+2b, 111a+2b, 112a+2b, 113a+2b, 114a+2b, 115a+2b, 116a+2b, 117a+2b, 118a+2b, 119a+2b, 120a+2b, 121a+2b, 122a+2b, 123a+2b, 124a+2b, 125a+2b, 126a+2b, 127a+2b, 128a+2b, 129a+2b, 130a+2b, 131a+2b, 132a+2b, 133a+2b, 134a+2b, 135a+2b, 136a+2b, 137a+2b, 138a+2b, 139a+2b, 140a+2b, 141a+2b, 142a+2b, 143a+2b, 144a+2b, 145a+2b, 146a+2b, 147a+2b, 148a+2b, 149a+2b, 150a+2b, 151a+2b, 152a+2b, 153a+2b, 154a+2b, 155a+2b, 156a+2b, 157a+2b, 158a+2b, 159a+2b, 160a+2b, 161a+2b, 162a+2b, 163a+2b, 164a+2b, 165a+2b, 166a+2b, 167a+2b, 168a+2b, 169a+2b, 170a+2b, 171a+2b, 172a+2b, 173a+2b, 174a+2b, 175a+2b, 176a+2b, 177a+2b, 178a+2b, 179a+2b, 180a+2b, 181a+2b, 182a+2b, 183a+2b, 184a+2b, 185a+2b, 186a+2b, 187a+2b, 188a+2b, 189a+2b, 190a+2b, 191a+2b, 192a+2b, 193a+2b, 194a+2b, 95a+2b, 96a+2b, 97a+2b, 98a+2b, 99a+2b, 200a+2b, 201a+2b, 202a+2b, 203a+2b, 204a+2b, 205a+2b, 206a+2b, 207a+2b, 208a+2b, 209a+2b, 210a+2b, 211a+2b, 212a+2b, 213a+2b, 214a+2b, 215a+2b, 216a+2b, 217a+2b, 218a+2b, 219a+2b, 220a+2b, 221a+2b, 222a+2b, 223a+2b, 224a+2b, 225a+2b, 226a+2b, 227a+2b, 228a+2b, 229a+2b, etc.

The present application, in various aspects, provides the following itemized embodiments:

1. A medical device, comprising an electrical device and an anti-scarring agent or a composition comprising an anti-scarring agent, wherein the agent inhibits scarring between the medical device and the host into which the medical device is implanted.

2. The medical device of item 1 wherein the agent is an adensosine A2A receptor antagonist.

3. The medical device of item 1 wherein the agent is an AKT inhibitor.

4. The medical device of item 1 wherein the agent is an alpha 2 integrin antagonist, wherein the alpha 2 integrin antagonist is Pharmaprojects No. 5754 (Merck KgaA).

5. The medical device of item 1 wherein the agent is an alpha 4 integrin antagonist.

6. The medical device of item 1 wherein the agent is an alpha 7 nicotinic receptor agonist.

7. The medical device of item 1 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

8. The medical device of item 1 wherein the agent is an apoptosis antagonist.

9. The medical device of item 1 wherein the agent is an apoptosis activator.

10. The medical device of item 1 wherein the agent is a beta 1 integrin antagonist.

11. The medical device of item 1 wherein the agent is a beta tubulin inhibitor.

12. The medical device of item 1 wherein the agent is a blocker of enzyme production in Hepatitis C.

13. The medical device of item 1 wherein the agent is a Bruton's tyrosine kinase inhibitor.

14. The medical device of item 1 wherein the agent is a calcineurin inhibitor.

15. The medical device of item 1 wherein the agent is a caspase 3 inhibitor.

16. The medical device of item 1 wherein the agent is a CC chemokine receptor antagonist.

17. The medical device of item 1 wherein the agent is a cell cycle inhibitor selected from the group consisting of SNS-595 (Sunesis), synthadotin, KRX-0403, and an analogue or derivative thereof.

18. The medical device of item 1 wherein the agent is a cathepsin B inhibitor.

19. The medical device of item 1 wherein the agent is a cathepsin K inhibitor, wherein the cathepsin K inhibitor is 462795 (GlaxoSmithKline), INPL-022-D6 (Amura Therapeutics), or an analogue or derivative thereof.

20. The medical device of item 1 wherein the agent is a cathepsin L inhibitor.

21. The medical device of item I wherein the agent is a CD40 antagonist.

22. The medical device of item 1 wherein the agent is a chemokine receptor agonist.

23. The medical device of item 1 wherein the agent is a chymase inhibitor.

24. The medical device of item I wherein the agent is a collagenase antagonist.

25. The medical device of item 1 wherein the agent is a CXCR antagonist.

26. The medical device of item 1 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

27. The medical device of item 1 wherein the agent is a cyclooxygenase 1 inhibitor.

28. The medical device of item 1 wherein the agent is a DHFR inhibitor.

29. The medical device of item 1 wherein the agent is a dual integrin inhibitor.

30. The medical device of item 1 wherein the agent is an elastase inhibitor.

31. The medical device of item 1 wherein the agent is an elongation factor-1 alpha inhibitor.

32. The medical device of item 1 wherein the agent is an endothelial growth factor antagonist.

33. The medical device of item 1 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

34. The medical device of item 1 wherein the agent is an endotoxin antagonist.

35. The medical device of item 1 wherein the agent is an epothilone and tubulin binder.

36. The medical device of item 1 wherein the agent is an estrogen receptor antagonist.

37. The medical device of item 1 wherein the agent is an FGF inhibitor.

38. The medical device of item 1 wherein the agent is a farnexyl transferase inhibitor.

39. The medical device of item 1 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

40. The medical device of item 1 wherein the agent is an FLT-3 kinase inhibitor.

41. The medical device of item 1 wherein the agent is an FGF receptor kinase inhibitor.

42. The medical device of item 1 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

43. The medical device of item 1 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

44. The medical device of item 1 wherein the agent is a histone deacetylase inhibitor.

45. The medical device of item 1 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

46. The medical device of item 1 wherein the agent is an ICAM inhibitor.

47. The medical device of item 1 wherein the agent is an IL, ICE and IRAK antagonist, wherein the antagonist is a CJ-14877, CP-424174 (Pfizer), NF-61 (Negma-Lerads), and an analogue or derivative thereof.

48. The medical device of item 1 wherein the agent is an IL-2 inhibitor.

49. The medical device of item 1 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

50. The medical device of item 1 wherein the agent is an IMPDH (inosine monophosphate).

51. The medical device of item 1 wherein the agent is an integrin antagonist.

52. The medical device of item 1 wherein the agent is an interleukin antagonist.

53. The medical device of item 1 wherein the agent is an inhibitor of type III receptor tyrosine kinase.

54. The medical device of item 1 wherein the agent is an irreversible inhibitor of enzyme methionine aminopeptidase type 2.

55. The medical device of item 1 wherein the agent is an isozyme selective delta protein kinase C inhibitor.

56. The medical device of item 1 wherein the agent a JAK3 enzyme inhibitor.

57. The medical device of item 1 wherein the agent is a JNK inhibitor.

58. The medical device of item 1 wherein the agent is a kinase inhibitor.

59. The medical device of item 1 wherein the agent is kinesin antagonist.

60. The medical device of item 1 wherein the agent is a kinesin antagonist.

61. The medical device of item 1 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

62. The medical device of item 1 wherein the agent is an MAP kinase inhibitor.

63. The medical device of item 1 wherein the agent is a matrix metalloproteinase inhibitor.

64. The medical device of item 1 wherein the agent is an MCP-CCR2 inhibitor.

65. The medical device of item 1 wherein the agent is an mTOR inhibitor.

66. The medical device of item 1 wherein the agent is an mTOR kinase inhibitor.

67. The medical device of item 1 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

68. The medical device of item 1 wherein the agent is an MIF inhibitor.

69. The medical device of item 1 wherein the agent is an MMP inhibitor.

70. The medical device of item 1 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

71. The medical device of item 1 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

72. The medical device of item 1 wherein the agent is a nitric oxide agonist.

73. The medical device of item 1 wherein the agent is an ornithine decarboxylase inhibitor.

74. The medical device of item 1 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

75. The medical device of item 1 wherein the agent is a palmitoyl-protein thioesterase inhibitor.

76. The medical device of item 1 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

77. The medical device of item 1 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-154), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

78. The medical device of item 1 wherein the agent is a phosphatase inhibitor.

79. The medical device of item 1 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

80. The medical device of item 1 wherein the agent is a PKC inhibitor.

81. The medical device of item 1 wherein the agent is a platelet activating factor antagonist.

82. The medical device of item 1 wherein the agent is a platelet-derived growth factor receptor kinase inhibitor.

83. The medical device of item 1 wherein the agent is a prolyl hydroxylase inhibitor.

84. The medical device of item 1 wherein the agent is a polymorphonuclear neutrophil inhibitor.

85. The medical device of item 1 wherein the agent is a protein kinase B inhibitor.

86. The medical device of item 1 wherein the agent is a protein kinase C stimulant.

87. The medical device of item 1 wherein the agent is a purine nucleoside analogue.

88. The medical device of item 1 wherein the agent is a purinoreceptor P2X antagonist.

89. The medical device of item 1 wherein the agent is a Raf kinase inhibitor.

90. The medical device of item 1 wherein the agent is a reversible inhibitor of ErbB1 and ErbB2.

91. The medical device of item 1 wherein the agent is a ribonucleoside triphosphate reductase inhibitor.

92. The medical device of item 1 wherein the agent is an SDF-1 antagonist.

93. The medical device of item 1 wherein the agent is a sheddase inhibitor.

94. The medical device of item 1 wherein the agent is an SRC inhibitor.

95. The medical device of item 1 wherein the agent is a stromelysin inhibitor.

96. The medical device of item 1 wherein the agent is an Syk kinase inhibitor.

97. The medical device of item 1 wherein the agent is a telomerase inhibitor.

98. The medical device of item 1 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

99. The medical device of item 1 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celetech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRX-119 (CombinatoRX), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

100. The medical device of item 1 wherein the agent is a Toll receptor inhibitor.

101. The medical device of item 1 wherein the agent is a tubulin antagonist.

102. The medical device of item 1 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

103. The medical device of item 1 wherein the agent is a VEGF inhibitor.

104. The medical device of item 1 wherein the agent is a vitamin D receptor agonist.

105. The medical device of item 1 wherein the agent is ZD-6474 (an angiogenesis inhibitor).

106. The medical device of item 1 wherein the agent is AP-23573 (an mTOR inhibitor).

107. The medical device of item 1 wherein the agent is synthadotin (a tubulin antagonist).

108. The medical device of item 1 wherein the agent is S-0885 (a collagenase inhibitor).

109. The medical device of item 1 wherein the agent is aplidine (an elongation factor-1 alpha inhibitor).

110. The medical device of item 1 wherein the agent is ixabepilone (an epithilone).

111. The medical device of item 1 wherein the agent is IDN-5390 (an angiogenesis inhibitor).

112. The medical device of item 1 wherein the agent is SB-2723005 (an angiogenesis inhibitor).

113. The medical device of item 1 wherein the agent is ABT-518 (an angiogenesis inhibitor).

114. The medical device of item 1 wherein the agent is combretastatin (an angiogenesis inhibitor).

115. The medical device of item 1 wherein the agent is anecortave acetate (an angiogenesis inhibitor).

116. The medical device of item 1 wherein the agent is SB-715992 (a kinesin antagonist).

117. The medical device of item 1 wherein the agent is temsirolimus (an mTOR inhibitor).

118. The medical device of item 1 wherein the agent is adalimumab (a TNFα antagonist).

119. The medical device of item 1, further comprising a coating, wherein the coating comprises the anti-scarring agent and a polymer.

120. The medical device of item 1, further comprising a coating, wherein the coating comprises the anti-scarring agent.

121. The medical device of item 1, further comprising a coating, wherein the coating is disposed on a surface of the electrical device.

122. The medical device of item 1, further comprising a coating, wherein the coating directly contacts the electrical device.

123. The medical device of item 1, further comprising a coating, wherein the coating indirectly contacts the electrical device.

124. The medical device of item 1, further comprising a coating, wherein the coating partially covers the electrical device.

125. The medical device of item 1, further comprising a coating, wherein the coating completely covers the electrical device.

126. The medical device of item 1, further comprising a coating, wherein the coating is a uniform coating.

127. The medical device of item 1, further comprising a coating, wherein the coating is a non-uniform coating.

128. The medical device of item 1, further comprising a coating, wherein the coating is a discontinuous coating.

129. The medical device of item 1, further comprising a coating, wherein the coating is a patterned coating.

130. The medical device of item 1, further comprising a coating, wherein the coating has a thickness of 100 μm or less.

131. The medical device of item 1, further comprising a coating, wherein the coating has a thickness of 10 μm or less.

132. The medical device of item 1, further comprising a coating, wherein the coating adheres to the surface of the electrical device upon deployment of the medical device.

133. The medical device of item 1, further comprising a coating, wherein the coating is stable at room temperature for a period of 1 year.

134. The medical device of item 1, further comprising a coating, wherein the anti-scarring agent is present in the coating in an amount ranging between about 0.0001% to about 1% by weight.

135. The medical device of item 1, further comprising a coating, wherein the anti-scarring agent is present in the coating in an amount ranging between about 1% to about 10% by weight.

136. The medical device of item 1, further comprising a coating, wherein the anti-scarring agent is present in the coating in an amount ranging between about 10% to about 25% by weight.

137. The medical device of item 1, further comprising a coating, wherein the anti-scarring agent is present in the coating in an amount ranging between about 25% to about 70% by weight.

138. The medical device of item 1, further comprising a coating, wherein the coating further comprises a polymer.

139. The medical device of item 1, further comprising a first coating having a first composition and the second coating having a second composition.

140. The medical device of item 1, further comprising a first coating having a first composition and the second coating having a second composition, wherein the first composition and the second composition are different.

141. The medical device of item 1, further comprising a polymer.

142. The medical device of item 1, further comprising a polymeric carrier.

143. The medical device of item 1, further comprising a polymeric carrier, wherein the polymeric carrier comprises a copolymer.

144. The medical device of item 1, further comprising a polymeric carrier, wherein the polymeric carrier comprises a block copolymer.

145. The medical device of item 1, further comprising a polymeric carrier, wherein the polymeric carrier comprises a random copolymer.

146. The medical device of item 1, further comprising a polymeric carrier, wherein the polymeric carrier comprises a biodegradable polymer.

147. The medical device of item 1, further comprising a polymeric carrier, wherein the polymeric carrier comprises a non-biodegradable polymer.

148. The medical device of item 1, further comprising a polymeric carrier, wherein the polymeric carrier comprises a hydrophilic polymer.

149. The medical device of item 1, further comprising a polymeric carrier, wherein the polymeric carrier comprises a hydrophobic polymer.

150. The medical device of item 1, further comprising a polymeric carrier, wherein the polymeric carrier comprises a polymer having hydrophilic domains.

151. The medical device of item 1, further comprising a polymeric carrier, wherein the polymeric carrier comprises a polymer having hydrophobic domains.

152. The medical device of item 1, further comprising a polymeric carrier, wherein the polymeric carrier comprises a non-conductive polymer.

153. The medical device of item 1, further comprising a polymeric carrier, wherein the polymeric carrier comprises an elastomer.

154. The medical device of item 1, further comprising a polymeric carrier, wherein the polymeric carrier comprises a hydrogel.

155. The medical device of item 1, further comprising a polymeric carrier, wherein the polymeric carrier comprises a silicone polymer.

156. The medical device of item 1, further comprising a polymeric carrier, wherein the polymeric carrier comprises a hydrocarbon polymer.

157. The medical device of item 1, further comprising a polymeric carrier, wherein the polymeric carrier comprises a styrene-derived polymer.

158. The medical device of item 1, further comprising a polymeric carrier, wherein the polymeric carrier comprises a butadiene polymer.

159. The medical device of item 1, further comprising a polymeric carrier, wherein the polymeric carrier comprises a macromer.

160. The medical device of item 1, further comprising a polymeric carrier, wherein the polymeric carrier comprises a poly(ethylene glycol) polymer.

161. The medical device of item 1, further comprising a polymeric carrier, wherein the polymeric carrier comprises an amorphous polymer.

162. The medical device of item 1, further comprising a lubricious coating.

163. The medical device of item 1 wherein the anti-scarring agent is located within pores or holes of the electrical device.

164. The medical device of item 1 wherein the anti-scarring agent is located within a channel, lumen, or divet of the electrical device.

165. The medical device of item 1, further comprising a second pharmaceutically active agent.

166. The medical device of item 1, further comprising an anti-inflammatory agent.

167. The medical device of item 1, further comprising an agent that inhibits infection.

168. The medical device of item 1, further comprising an agent that inhibits infection, wherein the agent is an anthracycline.

169. The medical device of item 1, further comprising an agent that inhibits infection, wherein the agent is doxorubicin.

170. The medical device of item 1, further comprising an agent that inhibits infection, wherein the agent is mitoxantrone.

171. The medical device of item 1, further comprising an agent that inhibits infection, wherein the agent is a fluoropyrimidine.

172. The medical device of item 1, further comprising an agent that inhibits infection, wherein the agent is 5-fluorouracil (5-FU).

173. The medical device of item 1, further comprising an agent that inhibits infection, wherein the agent is a folic acid antagonist.

174. The medical device of item 1, further comprising an agent that inhibits infection, wherein the agent is methotrexate.

175. The medical device of item 1, further comprising an agent that inhibits infection, wherein the agent is a podophylotoxin.

176. The medical device of item 1, further comprising an agent that inhibits infection, wherein the agent is etoposide

177. The medical device of item 1, further comprising an agent that inhibits infection, wherein the agent is a camptothecin.

178. The medical device of item 1, further comprising an agent that inhibits infection, wherein the agent is a hydroxyurea.

179. The medical device of item 1, further comprising an agent that inhibits infection, wherein the agent is a platinum complex.

180. The medical device of item 1, further comprising an agent that inhibits infection, wherein the agent is cisplatin.

181. The medical device of item 1, further comprising an anti-thrombotic agent.

182. The medical device of item 1, further comprising a visualization agent.

183. The medical device of item 1, further comprising a visualization agent, wherein the visualization agent is a radiopaque material, wherein the radiopaque material comprises a metal, a halogenated compound, or a barium containing compound.

184. The medical device of item 1, further comprising a visualization agent, wherein the visualization agent is a radiopaque material, wherein the radiopaque material comprises barium, tantalum, or technetium.

185. The medical device of item 1, further comprising a visualization agent, wherein the visualization agent is a MRI responsive material.

186. The medical device of item 1, further comprising a visualization agent, wherein the visualization agent comprises a gadolinium chelate.

187. The medical device of item 1, further comprising a visualization agent, wherein the visualization agent comprises iron, magnesium, manganese, copper, or chromium.

188. The medical device of item 1, further comprising a visualization agent, wherein the visualization agent comprises an iron oxide compound.

189. The medical device of item 1, further comprising a visualization agent, wherein the visualization agent comprises a dye, pigment, or colorant.

190. The medical device of item 1, further comprising an echogenic material.

191. The medical device of item 1, further comprising an echogenic material, wherein the echogenic material is in the form of a coating.

192. The medical device of item 1 wherein the device is sterile.

193. The medical device of item 1 wherein the anti-scarring agent inhibits adhesion between the medical device and a host into which the medical device is implanted.

194. The medical device of item 1 wherein the medical device delivers the anti-scarring agent locally to tissue proximate to the medical device.

195. The medical device of item 1 wherein the anti-scarring agent is released into tissue in the vicinity of the medical device after deployment of the medical device.

196. The medical device of item 1 wherein the anti-scarring agent is released into tissue in the vicinity of the medical device after deployment of the medical device, wherein the tissue is connective tissue.

197. The medical device of item 1 wherein the anti-scarring agent is released into tissue in the vicinity of the medical device after deployment of the medical device, wherein the tissue is muscle tissue.

198. The medical device of item 1 wherein the anti-scarring agent is released into tissue in the vicinity of the medical device after deployment of the medical device, wherein the tissue is nerve tissue.

199. The medical device of item 1 wherein the anti-scarring agent is released into tissue in the vicinity of the medical device after deployment of the medical device, wherein the tissue is epithelium tissue.

200. The medical device of item 1 wherein the anti-scarring agent is released in effective concentrations from the medical device over a period ranging from the time of deployment of the medical device to about 1 year.

201. The medical device of item 1 wherein the anti-scarring agent is released in effective concentrations from the medical device over a period ranging from about 1 month to 6 months.

202. The medical device of item 1 wherein the anti-scarring agent is released in effective concentrations from the medical device over a period ranging from about 1-90 days.

203. The medical device of item 1 wherein the anti-scarring agent is released in effective concentrations from the medical device at a constant rate.

204. The medical device of item 1 wherein the anti-scarring agent is released in effective concentrations from the medical device at an increasing rate.

205. The medical device of item 1 wherein the anti-scarring agent is released in effective concentrations from the medical device at a decreasing rate.

206. The medical device of item 1 wherein the anti-scarring agent is released in effective concentrations from the composition comprising the anti-scarring agent by diffusion over a period ranging from the time of deployment of the medical device to about 90 days.

207. The medical device of item 1 wherein the anti-scarring agent is released in effective concentrations from the composition comprising the anti-scarring agent by erosion of the composition over a period ranging from the time of deployment of the medical device to about 90 days.

208. The medical device of item 1 wherein the device comprises about 0.01 μg to about 10 μg of the anti-scarring agent.

209. The medical device of item 1 wherein the device comprises about 10 μg to about 10 mg of the anti-scarring agent.

210. The medical device of item 1 wherein the device comprises about 10 mg to about 250 mg of the anti-scarring agent.

211. The medical device of item 1 wherein the device comprises about 250 mg to about 1000 mg of the anti-scarring agent.

212. The medical device of item 1 wherein the device comprises about 1000 mg to about 2500 mg of the anti-scarring agent.

213. The medical device of item 1 wherein a surface of the device comprises less than 0.01 μg of the anti-scarring agent per mm² of device surface to which the anti-scarring agent is applied.

214. The medical device of item 1 wherein a surface of the device comprises about 0.01 μg to about 1 μg of the anti-scarring agent per mm² of device surface to which the anti-scarring agent is applied.

215. The medical device of item 1 wherein a surface of the device comprises about 1 μg to about 10 μg of the anti-scarring agent per mm² of device surface to which the anti-scarring agent is applied.

216. The medical device of item 1 wherein a surface of the device comprises about 10 μg to about 250 μg of the anti-scarring agent per mm² of device surface to which the anti-scarring agent is applied.

217. The medical device of item 1 wherein a surface of the device comprises about 250 μg to about 1000 μg of the anti-scarring agent of anti-scarring agent per mm² of device surface to which the anti-scarring agent is applied.

218. The medical device of item 1 wherein a surface of the device comprises about 1000 μg to about 2500 μg of the anti-scarring agent per mm² of device surface to which the anti-scarring agent is applied.

219. The medical device of item 1 wherein the agent or the composition is affixed to the electrical device.

220. The medical device of item 1 wherein the agent or the composition is covalently attached to the electrical device.

221. The medical device of item 1 wherein the agent or the composition is non-covalently attached to the electrical device.

222. The medical device of item 1 further comprising a coating that absorbs the agent or the composition.

223. The medical device of item 1 wherein the electrical device is interweaved with a thread composed of, or coated with, the agent or the composition.

224. The medical device of item 1 wherein a portion of the electrical device is covered with a sleeve that contains the agent or the composition.

225. The medical device of item 1 wherein the electrical device is completely covered with a sleeve that contains the agent or the composition.

226. The medical device of item 1 wherein a portion of the electrical device is covered with a mesh that contains the agent or the composition.

227. The medical device of item 1 wherein the electrical device is completely covered with a mesh that contains the agent or the composition.

228. The medical device of any one of items 1-227 wherein the electrical device is a neurostimulator.

229. The medical device of any one of items 1-227 wherein the electrical device is a spinal cord stimulator.

230. The medical device of any one of items 1-227 wherein the electrical device is a brain stimulator.

231. The medical device of any one of items 1-227 wherein the electrical device is a vagus nerve stimulator.

232. The medical device of any one of items 1-227 wherein the electrical device is a sacral nerve stimulator.

233. The medical device of any one of items 1-227 wherein the electrical device is a gastric nerve stimulator.

234. The medical device of any one of items 1-227 wherein the electrical device is an auditory nerve stimulator.

235. The medical device of any one of items 1-227 wherein the electrical device delivers stimulation to organs.

236. The medical device of any one of items 1-227 wherein the electrical device delivers stimulation to bone.

237. The medical device of any one of items 1-227 wherein the electrical device delivers stimulation to muscles.

238. The medical device of any one of items 1-227 wherein the electrical device delivers stimulation to tissues.

239. The medical device of any one of items 1-227 wherein the electrical device is a device for continuous subarachnoid infusion.

240. The medical device of any one of items 1-227 wherein the electrical device is an implantable electrode.

241. The medical device of any one of items 1-227 wherein the electrical device is an implantable pulse generator.

242. The medical device of any one of items 1-227 wherein the electrical device is an electrical lead.

243. The medical device of any one of items 1-227 wherein the electrical device is a stimulation lead.

244. The medical device of any one of items 1-227 wherein the electrical device is a simulation catheter lead.

245. The medical device of any one of items 1-227 wherein the electrical device is cochlear implant.

246. The medical device of any one of items 1-227 wherein the electrical device is a microstimulator.

247. The medical device of any one of items 1-227 wherein the electrical device is battery powered.

248. The medical device of any one of items 1-227 wherein the electrical device is radio frequency powered.

249. The medical device of any one of items 1-227 wherein the electrical device is both battery and radio frequency powered.

250. The medical device of any one of items 1-227 wherein the electrical device is a cardiac rhythm management device.

251. The medical device of any one of items 1-227 wherein the electrical device is a cardiac pacemaker.

252. The medical device of any one of items 1-227 wherein the electrical device is an implantable cardioverter defibrillator system.

253. The medical device of any one of items 1-227 wherein the electrical device is a cardiac lead.

254. The medical device of any one of items 1-227 wherein the electrical device is a pacer lead.

255. The medical device of any one of items 1-227 wherein the electrical device is an endocardial lead.

256. The medical device of any one of items 1-227 wherein the electrical device is a cardioversion/defibrillator lead.

257. The medical device of any one of items 1-227 wherein the electrical device is an epicardial lead.

258. The medical device of any one of items 1-227 wherein the electrical device is an epicardial defibrillator lead.

259. The medical device of any one of items 1-227 wherein the electrical device is a patch defibrillator.

260. The medical device of any one of items 1-227 wherein the electrical device is a patch defibrillator lead.

261. The medical device of any one of items 1-227 wherein the electrical device is an electrical patch.

262. The medical device of any one of items 1-227 wherein the electrical device is a transvenous lead.

263. The medical device of any one of items 1-227 wherein the electrical device is an active fixation lead.

264. The medical device of any one of items 1-227 wherein the electrical device is a passive fixation lead.

265. The medical device of any one of items 1-227 wherein the electrical device is a sensing lead.

266. The medical device of any one of items 1-227 wherein the electrical device is a defibrillator.

267. The medical device of any one of items 1-227 wherein the electrical device is an implantable sensor.

268. The medical device of any one of items 1-227 wherein the electrical device is a left ventricular assist device

269. The medical device of any one of items 1-227 wherein the electrical device is a pulse generator.

270. The medical device of any one of items 1-227 wherein the electrical device is a patch lead.

271. The medical device of any one of items 1-227 wherein the electrical device is an electrical patch.

272. The medical device of any one of items 1-227 wherein the electrical device is a cardiac stimulator.

273. The medical device of any one of items 1-227 wherein the electrical device is an electrical deviceable sensor.

274. The medical device of any one of items 1-227 wherein the electrical device is an electrical deviceable pump.

275. The medical device of any one of items 1-227 wherein the electrical device is a dural patch.

276. The medical device of any one of items 1-227 wherein the electrical device is a ventricular peritoneal shunt.

277. The medical device of any one of items 1-227 wherein the electrical device is a ventricular atrial shunt.

278. The medical device of any one of items 1-227 wherein the electrical device is adapted for treating or preventing epidural fibrosis post-laminectomy.

279. The medical device of any one of items 1-227 wherein the electrical device is adapted for treating or preventing cardiac rhythm abnormalities.

280. The medical device of any one of items 1-227 wherein the electrical device is adapted for treating or preventing atrial rhythm abnormalities

281. The medical device of any one of items 1-227 wherein the electrical device is adapted for treating or preventing conduction abnormalities.

282. The medical device of any one of items 1-227 wherein the electrical device is adapted for treating or preventing ventricular rhythm abnormalities.

283. The medical device of any one of items 1-227 wherein the electrical device is adapted for treating or preventing pain.

284. The medical device of any one of items 1-227 wherein the electrical device is adapted for treating or preventing epilepsy.

285. The medical device of any one of items 1-227 wherein the electrical device is adapted for treating or preventing Parkinson's disease.

286. The medical device of any one of items 1-227 wherein the electrical device is adapted for treating or preventing movement disorders.

287. The medical device of any one of items 1-227 wherein the electrical device is adapted for treating or preventing obesity.

288. The medical device of any one of items 1-227 wherein the electrical device is adapted for treating or preventing depression.

289. The medical device of any one of items 1-227 wherein the electrical device is adapted for treating or preventing anxiety.

290. The medical device of any one of items 1-227 wherein the electrical device is adapted for treating or preventing hearing loss.

291. The medical device of any one of items 1-227 wherein the electrical device is adapted for treating or preventing ulcers.

292. The medical device of any one of items 1-227 wherein the electrical device is adapted for treating or preventing deep vein thrombosis.

293. The medical device of any one of items 1-227 wherein the electrical device is adapted for treating or preventing muscular atrophy.

294. The medical device of any one of items 1-227 wherein the electrical device is adapted for treating or preventing joint stiffness.

295. The medical device of any one of items 1-227 wherein the electrical device is adapted for treating or preventing muscle spasms.

296. The medical device of any one of items 1-227 wherein the electrical device is adapted for treating or preventing osteoporosis.

297. The medical device of any one of items 1-227 wherein the electrical device is adapted for treating or preventing scoliosis.

298. The medical device of any one of items 1-227 wherein the electrical device is adapted for treating or preventing spinal disc degeneration.

299. The medical device of any one of items 1-227 wherein the electrical device is adapted for treating or preventing spinal cord injury.

300. The medical device of any one of items 1-227 wherein the electrical device is adapted for treating or preventing urinary dysfunction.

301. The medical device of any one of items 1-227 wherein the electrical device is adapted for treating or preventing gastroparesis.

302. The medical device of any one of items 1-227 wherein the electrical device is adapted for treating or preventing malignancy.

303. The medical device of any one of items 1-227 wherein the electrical device is adapted for treating or preventing arachnoiditis.

304. The medical device of any one of items 1-227 wherein the electrical device is adapted for treating or preventing chronic disease.

305. The medical device of any one of items 1-227 wherein the electrical device is adapted for treating or preventing migraine.

306. The medical device of any one of items 1-227 wherein the electrical device is adapted for treating or preventing sleep disorders.

307. The medical device of any one of items 1-227 wherein the electrical device is adapted for treating or preventing dementia.

308. The medical device of any one of items 1-227 wherein the electrical device is adapted for treating or preventing Alzheimer's disease.

309. A medical device, comprising a neurostimulator for treating chronic pain (i.e., an electrical device) and an anti-scarring agent or a composition comprising an anti-scarring agent, wherein the agent inhibits scarring between the medical device and the host into which the medical device is implanted.

310. The medical device of item 309 wherein the agent is an adensosine A2A receptor antagonist.

311. The medical device of item 309 wherein the agent is an AKT inhibitor.

312. The medical device of item 309 wherein the agent is an alpha 2 integrin antagonist, wherein the alpha 2 integrin antagonist is Pharmaprojects No. 5754 (Merck KgaA).

313. The medical device of item 309 wherein the agent is an alpha 4 integrin antagonist.

314. The medical device of item 309 wherein the agent is an alpha 7 nicotinic receptor agonist.

315. The medical device of item 309 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

316. The medical device of item 309 wherein the agent is an apoptosis antagonist.

317. The medical device of item 309 wherein the agent is an apoptosis activator.

318. The medical device of item 309 wherein the agent is a beta 1 integrin antagonist.

319. The medical device of item 309 wherein the agent is a beta tubulin inhibitor.

320. The medical device of item 309 wherein the agent is a blocker of enzyme production in Hepatitis C.

321. The medical device of item 309 wherein the agent is a Bruton's tyrosine kinase inhibitor.

322. The medical device of item 309 wherein the agent is a calcineurin inhibitor.

323. The medical device of item 309 wherein the agent is a caspase 3 inhibitor.

324. The medical device of item 309 wherein the agent is a CC chemokine receptor antagonist.

325. The medical device of item 309 wherein the agent is a cell cycle inhibitor selected from the group consisting of SNS-595 (Sunesis), synthadotin, KRX-0403, and an analogue or derivative thereof.

326. The medical device of item 309 wherein the agent is a cathepsin B inhibitor.

327. The medical device of item 309 wherein the agent is a cathepsin K inhibitor, wherein the cathepsin K inhibitor is 462795 (GlaxoSmithKline), INPL-022-D6 (Amura Therapeutics), or an analogue or derivative thereof.

328. The medical device of item 309 wherein the agent is a cathepsin L inhibitor.

329. The medical device of item 309 wherein the agent is a CD40 antagonist.

330. The medical device of item 309 wherein the agent is a chemokine receptor agonist.

331. The medical device of item 309 wherein the agent is a chymase inhibitor.

332. The medical device of item 309 wherein the agent is a collagenase antagonist.

333. The medical device of item 309 wherein the agent is a CXCR antagonist.

334. The medical device of item 309 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

335. The medical device of item 309 wherein the agent is a cyclooxygenase 1 inhibitor.

336. The medical device of item 309 wherein the agent is a DHFR inhibitor.

337. The medical device of item 309 wherein the agent is a dual integrin inhibitor.

338. The medical device of item 309 wherein the agent is an elastase inhibitor.

339. The medical device of item 309 wherein the agent is an elongation factor-1 alpha inhibitor.

340. The medical device of item 309 wherein the agent is an endothelial growth factor antagonist.

341. The medical device of item 309 wherein the agent is are endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

342. The medical device of item 309 wherein the agent is an endotoxin antagonist.

343. The medical device of item 309 wherein the agent is an epothilone and tubulin binder.

344. The medical device of item 309 wherein the agent is an estrogen receptor antagonist.

345. The medical device of item 309 wherein the agent is an FGF inhibitor.

346. The medical device of item 309 wherein the agent is a farnexyl transferase inhibitor.

347. The medical device of item 309 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

348. The medical device of item 309 wherein the agent is an FLT-3 kinase inhibitor.

349. The medical device of item 309 wherein the agent is an FGF receptor kinase inhibitor.

350. The medical device of item 309 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

351. The medical device of item 309 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

352. The medical device of item 309 wherein the agent is a histone deacetylase inhibitor.

353. The medical device of item 309 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

354. The medical device of item 309 wherein the agent is an ICAM inhibitor.

355. The medical device of item 309 wherein the agent is an IL, ICE and IRAK antagonist, wherein the antagonist is a CJ-14877, CP-424174 (Pfizer), NF-61 (Negma-Lerads), and an analogue or derivative thereof.

356. The medical device of item 309 wherein the agent is an IL-2 inhibitor.

357. The medical device of item 309 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

358. The medical device of item 309 wherein the agent is an IMPDH (inosine monophosphate)

359. The medical device of item 309 wherein the agent is an integrin antagonist.

360. The medical device of item 309 wherein the agent is an interleukin antagonist.

361. The medical device of item 309 wherein the agent is an inhibitor of type III receptor tyrosine kinase.

362. The medical device of item 309 wherein the agent is an irreversible inhibitor of enzyme methionine aminopeptidase type 2.

363. The medical device of item 309 wherein the agent is an isozyme selective delta protein kinase C inhibitor.

364. The medical device of item 309 wherein the agent a JAK3 enzyme inhibitor.

365. The medical device of item 309 wherein the agent is a JNK inhibitor.

366. The medical device of item 309 wherein the agent is a kinase inhibitor.

367. The medical device of item 309 wherein the agent is kinesin antagonist.

368. The medical device of item 309 wherein the agent is a kinesin antagonist.

369. The medical device of item 309 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

370. The medical device of item 309 wherein the agent is an MAP kinase inhibitor.

371. The medical device of item 309 wherein the agent is a matrix metalloproteinase inhibitor.

372. The medical device of item 309 wherein the agent is an MCP-CCR2 inhibitor.

373. The medical device of item 309 wherein the agent is an mTOR inhibitor.

374. The medical device of item 309 wherein the agent is an mTOR kinase inhibitor.

375. The medical device of item 309 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof

376. The medical device of item 309 wherein the agent is an MIF inhibitor

377. The medical device of item 309 wherein the agent is an MMP inhibitor.

378. The medical device of item 309 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006,4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

379. The medical device of item 309 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

380. The medical device of item 309 wherein the agent is a nitric oxide agonist.

381. The medical device of item 309 wherein the agent is an ornithine decarboxylase inhibitor

382. The medical device of item 309 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

383. The medical device of item 309 wherein the agent is a palmitoyl-protein thioesterase inhibitor

384. The medical device of item 309 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

385. The medical device of item 309 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

386. The medical device of item 309 wherein the agent is a phosphatase inhibitor.

387. The medical device of item 309 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No-2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

388. The medical device of item 309 wherein the agent is a PKC inhibitor.

389. The medical device of item 309 wherein the agent is a platelet activating factor antagonist.

390. The medical device of item 309 wherein the agent is a platelet-derived growth factor receptor kinase inhibitor.

391. The medical device of item 309 wherein the agent is a prolyl hydroxylase inhibitor.

392. The medical device of item 309 wherein the agent is a polymorphonuclear neutrophil inhibitor.

393. The medical device of item 309 wherein the agent is a protein kinase B inhibitor.

394. The medical device of item 309 wherein the agent is a protein kinase C stimulant.

395. The medical device of item 309 wherein the agent is a purine nucleoside analogue.

396. The medical device of item 309 wherein the agent is a purinoreceptor P2X antagonist.

397. The medical device of item 309 wherein the agent is a Raf kinase inhibitor.

398. The medical device of item 309 wherein the agent is a reversible inhibitor of ErbB1 and ErbB2.

399. The medical device of item 309 wherein the agent is a ribonucleoside triphosphate reductase inhibitor.

400. The medical device of item 309 wherein the agent is an SDF-1 antagonist.

401. The medical device of item 309 wherein the agent is a sheddase inhibitor.

402. The medical device of item 309 wherein the agent is an SRC inhibitor.

403. The medical device of item 309 wherein the agent is a stromelysin inhibitor.

404. The medical device of item 309 wherein the agent is an Syk kinase inhibitor.

405. The medical device of item 309 wherein the agent is a telomerase inhibitor.

406. The medical device of item 309 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

407. The medical device of item 309 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

408. The medical device of item 309 wherein the agent is a Toll receptor inhibitor.

409. The medical device of item 309 wherein the agent is a tubulin antagonist.

410. The medical device of item 309 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

411. The medical device of item 309 wherein the agent is a VEGF inhibitor.

412. The medical device of item 309 wherein the agent is a vitamin D receptor agonist.

413. The medical device of item 309 wherein the agent is ZD-6474 (an angiogenesis inhibitor).

414. The medical device of item 309 wherein the agent is AP-23573 (an mTOR inhibitor).

415. The medical device of item 309 wherein the agent is synthadotin (a tubulin antagonist).

416. The medical device of item 309 wherein the agent is S-0885 (a collagenase inhibitor).

417. The medical device of item 309 wherein the agent is aplidine (an elongation factor-1 alpha inhibitor).

418. The medical device of item 309 wherein the agent is ixabepilone (an epithilone).

419. The medical device of item 309 wherein the agent is IDN-5390 (an angiogenesis inhibitor).

420. The medical device of item 309 wherein the agent is SB-2723005 (an angiogenesis inhibitor).

421. The medical device of item 309 wherein the agent is ABT-518 (an angiogenesis inhibitor).

422. The medical device of item 309 wherein the agent is combretastatin (an angiogenesis inhibitor).

423. The medical device of item 309 wherein the agent is anecortave acetate (an angiogenesis inhibitor).

424. The medical device of item 309 wherein the agent is SB-715992 (a kinesin antagonist).

425. The medical device of item 309 wherein the agent is temsirolimus (an mTOR inhibitor).

426. The medical device of item 309 wherein the agent is adalimumab (a TNFα antagonist).

427. The medical device of item 309, further comprising a coating, wherein the coating comprises the anti-scarring agent and a polymer.

428. The medical device of item 309, further comprising a coating, wherein the coating comprises the anti-scarring agent.

429. The medical device of item 309, further comprising a coating, wherein the coating is disposed on a surface of the electrical device.

430. The medical device of item 309, further comprising a coating, wherein the coating directly contacts the electrical device.

431. The medical device of item 309, further comprising a coating, wherein the coating indirectly contacts the electrical device.

432. The medical device of item 309, further comprising a coating, wherein the coating partially covers the electrical device.

433. The medical device of item 309, further comprising a coating, wherein the coating completely covers the electrical device.

434. The medical device of item 309, further comprising a coating, wherein the coating is a uniform coating.

435. The medical device of item 309, further comprising a coating, wherein the coating is a non-uniform coating.

436. The medical device of item 309, further comprising a coating, wherein the coating is a discontinuous coating.

437. The medical device of item 309, further comprising a coating, wherein the coating is a patterned coating.

438. The medical device of item 309, further comprising a coating, wherein the coating has a thickness of 100 μm or less.

439. The medical device of item 309, further comprising a coating, wherein the coating has a thickness of 10 μm or less.

440. The medical device of item 309, further comprising a coating, wherein the coating adheres to the surface of the electrical device upon deployment of the medical device.

441. The medical device of item 309, further comprising a coating, wherein the coating is stable at room temperature for a period of 1 year.

442. The medical device of item 309, further comprising a coating, wherein the anti-scarring agent is present in the coating in an amount ranging between about 0.0001% to about 1% by weight.

443. The medical device of item 309, further comprising a coating, wherein the anti-scarring agent is present in the coating in an amount ranging between about 1% to about 10% by weight.

444. The medical device of item 309, further comprising a coating, wherein the anti-scarring agent is present in the coating in an amount ranging between about 10% to about 25% by weight.

445. The medical device of item 309, further comprising a coating, wherein the anti-scarring agent is present in the coating in an amount ranging between about 25% to about 70% by weight.

446. The medical device of item 309, further comprising a coating, wherein the coating further comprises a polymer.

447. The medical device of item 309, further comprising a first coating having a first composition and the second coating having a second composition.

448. The medical device of item 309, further comprising a first coating having a first composition and the second coating having a second composition, wherein the first composition and the second composition are different.

449. The medical device of item 309, further comprising a polymer.

450. The medical device of item 309, further comprising a polymeric carrier.

451. The medical device of item 309, further comprising a polymeric carrier, wherein the polymeric carrier comprises a copolymer.

452. The medical device of item 309, further comprising a polymeric carrier, wherein the polymeric carrier comprises a block copolymer.

453. The medical device of item 309, further comprising a polymeric carrier, wherein the polymeric carrier comprises a random copolymer.

454. The medical device of item 309, further comprising a polymeric carrier, wherein the polymeric carrier comprises a biodegradable polymer.

455. The medical device of item 309, further comprising a polymeric carrier, wherein the polymeric carrier comprises a non-biodegradable polymer.

456. The medical device of item 309, further comprising a polymeric carrier, wherein the polymeric carrier comprises a hydrophilic polymer.

457. The medical device of item 309, further comprising a polymeric carrier, wherein the polymeric carrier comprises a hydrophobic polymer.

458. The medical device of item 309, further comprising a polymeric carrier, wherein the polymeric carrier comprises a polymer having hydrophilic domains.

459. The medical device of item 309, further comprising a polymeric carrier, wherein the polymeric carrier comprises a polymer having hydrophobic domains.

460. The medical device of item 309, further comprising a polymeric carrier, wherein the polymeric carrier comprises a non-conductive polymer.

461. The medical device of item 309, further comprising a polymeric carrier, wherein the polymeric carrier comprises an elastomer.

462. The medical device of item 309, further comprising a polymeric carrier, wherein the polymeric carrier comprises a hydrogel.

463. The medical device of item 309, further comprising a polymeric carrier, wherein the polymeric carrier comprises a silicone polymer.

464. The medical device of item 309, further comprising a polymeric carrier, wherein the polymeric carrier comprises a hydrocarbon polymer.

465. The medical device of item 309, further comprising a polymeric carrier, wherein the polymeric carrier comprises a styrene-derived polymer.

466. The medical device of item 309, further comprising a polymeric carrier, wherein the polymeric carrier comprises a butadiene polymer.

467. The medical device of item 309, further comprising a polymeric carrier, wherein the polymeric carrier comprises a macromer.

468. The medical device of item 309, further comprising a polymeric carrier, wherein the polymeric carrier comprises a poly(ethylene glycol) polymer.

469. The medical device of item 309, further comprising a polymeric carrier, wherein the polymeric carrier comprises an amorphous polymer.

470. The medical device of item 309, further comprising a lubricious coating.

471. The medical device of item 309 wherein the anti-scarring agent is located within pores or holes of the electrical device.

472. The medical device of item 309 wherein the anti-scarring agent is located within a channel, lumen, or divet of the electrical device.

473. The medical device of item 309, further comprising a second pharmaceutically active agent.

474. The medical device of item 309, further comprising an anti-inflammatory agent.

475. The medical device of item 309, further comprising an agent that inhibits infection.

476. The medical device of item 309, further comprising an agent that inhibits infection, wherein the agent is an anthracycline.

477. The medical device of item 309, further comprising an agent that inhibits infection, wherein the agent is doxorubicin.

478. The medical device of item 309, further comprising an agent that inhibits infection, wherein the agent is mitoxantrone.

479. The medical device of item 309, further comprising an agent that inhibits infection, wherein the agent is a fluoropyrimidine.

480. The medical device of item 309, further comprising an agent that inhibits infection, wherein the agent is 5-fluorouracil (5-FU).

481. The medical device of item 309, further comprising an agent that inhibits infection, wherein the agent is a folic acid antagonist.

482. The medical device of item 309, further comprising an agent that inhibits infection, wherein the agent is methotrexate.

483. The medical device of item 309, further comprising an agent that inhibits infection, wherein the agent is a podophylotoxin.

484. The medical device of item 309, further comprising an agent that inhibits infection, wherein the agent is etoposide.

485. The medical device of item 309, further comprising an agent that inhibits infection, wherein the agent is a camptothecin.

486. The medical device of item 309, further comprising an agent that inhibits infection, wherein the agent is a hydroxyurea.

487. The medical device of item 309, further comprising an agent that inhibits infection, wherein the agent is a platinum complex.

488. The medical device of item 309, further comprising an agent that inhibits infection, wherein the agent is cisplatin.

489. The medical device of item 309, further comprising an anti-thrombotic agent.

490. The medical device of item 309, further comprising a visualization agent.

491. The medical device of item 309, further comprising a visualization agent, wherein the visualization agent is a radiopaque material, wherein the radiopaque material comprises a metal, a halogenated compound, or a barium containing compound.

492. The medical device of item 309, further comprising a visualization agent, wherein the visualization agent is a radiopaque material, wherein the radiopaque material comprises barium, tantalum, or technetium.

493. The medical device of item 309, further comprising a visualization agent, wherein the visualization agent is a MRI responsive material.

494. The medical device of item 309, further comprising a visualization agent, wherein the visualization agent comprises a gadolinium chelate.

495. The medical device of item 309, further comprising a visualization agent, wherein the visualization agent comprises iron, magnesium, manganese, copper, or chromium.

496. The medical device of item 309, further comprising a visualization agent, wherein the visualization agent comprises an iron oxide compound.

497. The medical device of item 309, further comprising a visualization agent, wherein the visualization agent comprises a dye, pigment, or colorant.

498. The medical device of item 309, further comprising an echogenic material.

499. The medical device of item 309, further comprising an echogenic material, wherein the echogenic material is in the form of a coating

500. The medical device of item 309 wherein the device is sterile.

501. The medical device of item 309 wherein the anti-scarring agent inhibits adhesion between the medical device and a host into which the medical device is implanted.

502. The medical device of item 309 wherein the medical device delivers the anti-scarring agent locally to tissue proximate to the medical device.

503. The medical device of item 309 wherein the anti-scarring agent is released into tissue in the vicinity of the medical device after deployment of the medical device.

504. The medical device of item 309 wherein the anti-scarring agent is released into tissue in the vicinity of the medical device after deployment of the medical device, wherein the tissue is connective tissue.

505. The medical device of item 309 wherein the anti-scarring agent is released into tissue in the vicinity of the medical device after deployment of the medical device, wherein the tissue is muscle tissue.

506. The medical device of item 309 wherein the anti-scarring agent is released into tissue in the vicinity of the medical device after deployment of the medical device, wherein the tissue is nerve tissue.

507. The medical device of item 309 wherein the anti-scarring agent is released into tissue in the vicinity of the medical device after deployment of the medical device, wherein the tissue is epithelium tissue.

508. The medical device of item 309 wherein the anti-scarring agent is released in effective concentrations from the medical device over a period ranging from the time of deployment of the medical device to about 1 year.

509. The medical device of item 309 wherein the anti-scarring agent is released in effective concentrations from the medical device over a period ranging from about 1 month to 6 months.

510. The medical device of item 309 wherein the anti-scarring agent is released in effective concentrations from the medical device over a period ranging from about 1-90 days.

511. The medical device of item 309 wherein the anti-scarring agent is released in effective concentrations from the medical device at a constant rate.

512. The medical device of item 309 wherein the anti-scarring agent is released in effective concentrations from the medical device at an increasing rate.

513. The medical device of item 309 wherein the anti-scarring agent is released in effective concentrations from the medical device at a decreasing rate.

514. The medical device of item 309 wherein the anti-scarring agent is released in effective concentrations from the composition comprising the anti-scarring agent by diffusion over a period ranging from the time of deployment of the medical device to about 90 days.

515. The medical device of item 309 wherein the anti-scarring agent is released in effective concentrations from the composition comprising the anti-scarring agent by erosion of the composition over a period ranging from the time of deployment of the medical device to about 90 days.

516. The medical device of item 309 wherein the device comprises about 0.01 μg to about 10 μg of the anti-scarring agent.

517. The medical device of item 309 wherein the device comprises about 10 μg to about 10 mg of the anti-scarring agent.

518. The medical device of item 309 wherein the device comprises about 10 mg to about 250 mg of the anti-scarring agent.

519. The medical device of item 309 wherein the device comprises about 250 mg to about 1000 mg of the anti-scarring agent.

520. The medical device of item 309 wherein the device comprises about 1000 mg to about 2500 mg of the anti-scarring agent.

521. The medical device of item 309 wherein a surface of the device comprises less than 0.01 μg of the anti-scarring agent per mm² of device surface to which the anti-scarring agent is applied.

522. The medical device of item 309 wherein a surface of the device comprises about 0.01 μg to about 1 μg of the anti-scarring agent per mm² of device surface to which the anti-scarring agent is applied.

523. The medical device of item 309 wherein a surface of the device comprises about 1 μg to about 10 μg of the anti-scarring agent per mm² of device surface to which the anti-scarring agent is applied.

524. The medical device of item 309 wherein a surface of the device comprises about 10 μg to about 250 μg of the anti-scarring agent per mm² of device surface to which the anti-scarring agent is applied.

525. The medical device of item 309 wherein a surface of the device comprises about 250 μg to about 1000 μg of the anti-scarring agent of anti-scarring agent per mm² of device surface to which the anti-scarring agent is applied.

526. The medical device of item 309 wherein a surface of the device comprises about 1000 μg to about 2500 μg of the anti-scarring agent per mm² of device surface to which the anti-scarring agent is applied.

527. The medical device of item 309 wherein the agent or the composition is affixed to the electrical device.

528. The medical device of item 309 wherein the agent or the composition is covalently attached to the electrical device.

529. The medical device of item 309 wherein the agent or the composition is non-covalently attached to the electrical device.

530. The medical device of item 309 further comprising a coating that absorbs the agent or the composition.

531. The medical device of item 309 wherein the electrical device is interweaved with a thread composed of, or coated with, the agent or the composition.

532. The medical device of item 309 wherein a portion of the electrical device is covered with a sleeve that contains the agent or the composition.

533. The medical device of item 309 wherein the electrical device is completely covered with a sleeve that contains the agent or the composition.

534. The medical device of item 309 wherein a portion of the electrical device is covered with a mesh that contains the agent or the composition.

535. The medical device of item 309 wherein the electrical device is completely covered with a mesh that contains the agent or the composition.

536. The medical device of items 309-535 wherein the chronic pain results from injury.

537. The medical device of items 309-535 wherein the chronic pain results from an illness.

538. The medical device of items 309-535 wherein the chronic pain results from scoliosis.

539. The medical device of items 309-535 wherein the chronic pain results from spinal disc degeneration.

540. The medical device of items 309-535 wherein the chronic pain results from malignancy.

541. The medical device of items 309-535 wherein the chronic pain results from arachnoiditis.

542. The medical device of items 309-535 wherein the chronic pain results from a chronic disease.

543. The medical device of items 309-535 wherein the chronic pain results from a pain syndrome.

544. The medical device of items 309-535 wherein the neurostimulator comprises a lead that delivers electrical stimulation to a nerve and an electrical connection that connects a power source to the lead.

545. The medical device of items 309-535 wherein the neurostimulator is adapted for spinal cord stimulation, and comprises a sensor that detects the position of the spine and a stimulator that emits pulses that decrease in amplitude when the back is in a supine position.

546. The medical device of items 309-535 wherein the neurostimulator comprises an electrode and a control circuit that generates pulses and rest period based on intervals corresponding to the host body's activity and regeneration period.

547. The medical device of items 309-535 wherein the neurostimulator comprises a stimulation catheter lead and an electrode.

548. The medical device of items 309-535 wherein the neurostimulator is a self-centering epidural spinal cord lead.

549. A medical device, comprising a neurostimulator for treating Parkinson's Disease (i.e., an electrical device) and an anti-scarring agent or a composition comprising an anti-scarring agent, wherein the agent inhibits scarring between the medical device and the host into which the medical device is implanted.

550. The medical device of item 549 wherein the agent is an adensosine A2A receptor antagonist.

551. The medical device of item 549 wherein the agent is an AKT inhibitor.

552. The medical device of item 549 wherein the agent is an alpha 2 integrin antagonist, wherein the alpha 2 integrin antagonist is Pharmaprojects No. 5754 (Merck KgaA).

553. The medical device of item 549 wherein the agent is an alpha 4 integrin antagonist.

554. The medical device of item 549 wherein the agent is an alpha 7 nicotinic receptor agonist.

555. The medical device of item 549 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

556. The medical device of item 549 wherein the agent is an apoptosis antagonist.

557. The medical device of item 549 wherein the agent is an apoptosis activator.

558. The medical device of item 549 wherein the agent is a beta 1 integrin antagonist.

559. The medical device of item 549 wherein the agent is a beta tubulin inhibitor.

560. The medical device of item 549 wherein the agent is a blocker of enzyme production in Hepatitis C.

561. The medical device of item 549 wherein the agent is a Bruton's tyrosine kinase inhibitor.

562. The medical device of item 549 wherein the agent is a calcineurin inhibitor.

563. The medical device of item 549 wherein the agent is a caspase 3 inhibitor.

564. The medical device of item 549 wherein the agent is a CC chemokine receptor antagonist.

565. The medical device of item 549 wherein the agent is a cell cycle inhibitor selected from the group consisting of SNS-595 (Sunesis), synthadotin, KRX-0403, and an analogue or derivative thereof.

566. The medical device of item 549 wherein the agent is a cathepsin B inhibitor.

567. The medical device of item 549 wherein the agent is a cathepsin K inhibitor, wherein the cathepsin K inhibitor is 462795 (GlaxoSmithKline), INPL-022-D6 (Amura Therapeutics), or an analogue or derivative thereof.

568. The medical device of item 549 wherein the agent is a cathepsin L inhibitor.

569. The medical device of item 549 wherein the agent is a CD40 antagonist.

570. The medical device of item 549 wherein the agent is a chemokine receptor agonist.

571. The medical device of item 549 wherein the agent is a chymase inhibitor.

572. The medical device of item 549 wherein the agent is a collagenase antagonist.

573. The medical device of item 549 wherein the agent is a CXCR antagonist.

574. The medical device of item 549 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

575. The medical device of item 549 wherein the agent is a cyclooxygenase 1 inhibitor.

576. The medical device of item 549 wherein the agent is a DHFR inhibitor.

577. The medical device of item 549 wherein the agent is a dual integrin inhibitor.

578. The medical device of item 549 wherein the agent is an elastase inhibitor.

579. The medical device of item 549 wherein the agent is an elongation factor-1 alpha inhibitor.

580. The medical device of item 549 wherein the agent is an endothelial growth factor antagonist.

581. The medical device of item 549 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

582. The medical device of item 549 wherein the agent is an endotoxin antagonist.

583. The medical device of item 549 wherein the agent is an epothilone and tubulin binder.

584. The medical device of item 549 wherein the agent is an estrogen receptor antagonist.

585. The medical device of item 549 wherein the agent is an FGF inhibitor.

586. The medical device of item 549 wherein the agent is a farnexyl transferase inhibitor.

587. The medical device of item 549 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

588. The medical device of item 549 wherein the agent is an FLT-3 kinase inhibitor.

589. The medical device of item 549 wherein the agent is an FGF receptor kinase inhibitor.

590. The medical device of item 549 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

591. The medical device of item 549 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

592. The medical device of item 549 wherein the agent is a histone deacetylase inhibitor.

593. The medical device of item 549 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

594. The medical device of item 549 wherein the agent is an ICAM inhibitor.

595. The medical device of item 549 wherein the agent is an IL, ICE and IRAK antagonist, wherein the antagonist is a CJ-14877, CP-424174 (Pfizer), NF-61 (Negma-Lerads), and an analogue or derivative thereof.

596. The medical device of item 549 wherein the agent is an IL-2 inhibitor.

597. The medical device of item 549 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

598. The medical device of item 549 wherein the agent is an IMPDH (inosine monophosphate).

599. The medical device of item 549 wherein the agent is an integrin antagonist.

600. The medical device of item 549 wherein the agent is an interleukin antagonist.

601. The medical device of item 549 wherein the agent is an inhibitor of type III receptor tyrosine kinase.

602. The medical device of item 549 wherein the agent is an irreversible inhibitor of enzyme methionine aminopeptidase type 2.

603. The medical device of item 549 wherein the agent is an isozyme selective delta protein kinase C inhibitor.

604. The medical device of item 549 wherein the agent a JAK3 enzyme inhibitor.

605. The medical device of item 549 wherein the agent is a JNK inhibitor.

606. The medical device of item 549 wherein the agent is a kinase inhibitor.

607. The medical device of item 549 wherein the agent is kinesin antagonist.

608. The medical device of item 549 wherein the agent is a kinesin antagonist.

609. The medical device of item 549 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

610. The medical device of item 549 wherein the agent is an MAP kinase inhibitor.

611. The medical device of item 549 wherein the agent is a matrix metalloproteinase inhibitor.

612. The medical device of item 549 wherein the agent is an MCPG-CCR2 inhibitor.

613. The medical device of item 549 wherein the agent is an mTOR inhibitor.

614. The medical device of item 549 wherein the agent is an mTOR kinase inhibitor.

615. The medical device of item 549 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

616. The medical device of item 549 wherein the agent is an MIF inhibitor.

617. The medical device of item 549 wherein the agent is an MMP inhibitor.

618. The medical device of item 549 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

619. The medical device of item 549 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

620. The medical device of item 549 wherein the agent is a nitric oxide agonist.

621. The medical device of item 549 wherein the agent is an ornithine decarboxylase inhibitor.

622. The medical device of item 549 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

623. The medical device of item 549 wherein the agent is a palmitoyl-protein thioesterase inhibitor.

624. The medical device of item 549 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

625. The medical device of item 549 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

626. The medical device of item 549 wherein the agent is a phosphatase inhibitor.

627. The medical device of item 549 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-2 4 8 7 0, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

628. The medical device of item 549 wherein the agent is a PKC inhibitor.

629. The medical device of item 549 wherein the agent is a platelet activating factor antagonist.

630. The medical device of item 549 wherein the agent is a platelet-derived growth factor receptor kinase inhibitor.

631. The medical device of item 549 wherein the agent is a prolyl hydroxylase inhibitor.

632. The medical device of item 549 wherein the agent is a polymorphonuclear neutrophil inhibitor.

633. The medical device of item 549 wherein the agent is a protein kinase B inhibitor.

634. The medical device of item 549 wherein the agent is a protein kinase C stimulant.

635. The medical device of item 549 wherein the agent is a purine nucleoside analogue.

636. The medical device of item 549 wherein the agent is a purinoreceptor P2X antagonist.

637. The medical device of item 549 wherein the agent is a Raf kinase inhibitor.

638. The medical device of item 549 wherein the agent is a reversible inhibitor of ErbB1 and ErbB2.

639. The medical device of item 549 wherein the agent is a ribonucleoside triphosphate reductase inhibitor.

640. The medical device of item 549 wherein the agent is an SDF-1 antagonist.

641. The medical device of item 549 wherein the agent is a sheddase inhibitor.

642. The medical device of item 549 wherein the agent is an SRC inhibitor.

643. The medical device of item 549 wherein the agent is a stromelysin inhibitor.

644. The medical device of item 549 wherein the agent is an Syk kinase inhibitor.

645. The medical device of item 549 wherein the agent is a telomerase inhibitor.

646. The medical device of item 549 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

647. The medical device of item 549 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

648. The medical device of item 549 wherein the agent is a Toll receptor inhibitor.

649. The medical device of item 549 wherein the agent is a tubulin antagonist.

650. The medical device of item 549 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

651. The medical device of item 549 wherein the agent is a VEGF inhibitor.

652. The medical device of item 549 wherein the agent is a vitamin D receptor agonist.

653. The medical device of item 549 wherein the agent is ZD-6474 (an angiogenesis inhibitor).

654. The medical device of item 549 wherein the agent is AP-23573 (an mTOR inhibitor).

655. The medical device of item 549 wherein the agent is synthadotin (a tubulin antagonist).

656. The medical device of item 549 wherein the agent is S-0885 (a collagenase inhibitor).

657. The medical device of item 549 wherein the agent is aplidine (an elongation factor-1 alpha inhibitor).

658. The medical device of item 549 wherein the agent is ixabepilone (an epithilone).

659. The medical device of item 549 wherein the agent is IDN-5390 (an angiogenesis inhibitor).

660. The medical device of item 549 wherein the agent is SB-2723005 (an angiogenesis inhibitor).

661. The medical device of item 549 wherein the agent is ABT-518 (an angiogenesis inhibitor).

662. The medical device of item 549 wherein the agent is combretastatin (an angiogenesis inhibitor).

663. The medical device of item 549 wherein the agent is anecortave acetate (an angiogenesis inhibitor).

664. The medical device of item 549 wherein the agent is SB-715992 (a kinesin antagonist).

665. The medical device of item 549 wherein the agent is temsirolimus (an mTOR inhibitor).

666. The medical device of item 549 wherein the agent is adalimumab (a TNFα antagonist).

667. The medical device of item 549, further comprising a coating, wherein the coating comprises the anti-scarring agent and a polymer.

668. The medical device of item 549, further comprising a coating, wherein the coating comprises the anti-scarring agent.

669. The medical device of item 549, further comprising a coating, wherein the coating is disposed on a surface of the electrical device.

670. The medical device of item 549, further comprising a coating, wherein the coating directly contacts the electrical device.

671. The medical device of item 549, further comprising a coating, wherein the coating indirectly contacts the electrical device.

672. The medical device of item 549, further comprising a coating, wherein the coating partially covers the electrical device.

673. The medical device of item 549, further comprising a coating, wherein the coating completely covers the electrical device.

674. The medical device of item 549, further comprising a coating, wherein the coating is a uniform coating.

675. The medical device of item 549, further comprising a coating, wherein the coating is a non-uniform coating.

676. The medical device of item 549, further comprising a coating, wherein the coating is a discontinuous coating.

677. The medical device of item 549, further comprising a coating, wherein the coating is a patterned coating.

678. The medical device of item 549, further comprising a coating, wherein the coating has a thickness of 100 μm or less.

679. The medical device of item 549, further comprising a coating, wherein the coating has a thickness of 10 μm or less.

680. The medical device of item 549, further comprising a coating, wherein the coating adheres to the surface of the electrical device upon deployment of the medical device.

681. The medical device of item 549, further comprising a coating, wherein the coating is stable at room temperature for a period of 1 year.

682. The medical device of item 549, further comprising a coating, wherein the anti-scarring agent is present in the coating in an amount ranging between about 0.0001% to about 1% by weight.

683. The medical device of item 549, further comprising a coating, wherein the anti-scarring agent is present in the coating in an amount ranging between about 1% to about 10% by weight.

684. The medical device of item 549, further comprising a coating, wherein the anti-scarring agent is present in the coating in an amount ranging between about 10% to about 25% by weight.

685. The medical device of item 549, further comprising a coating, wherein the anti-scarring agent is present in the coating in an amount ranging between about 25% to about 70% by weight.

686. The medical device of item 549, further comprising a coating, wherein the coating further comprises a polymer.

687. The medical device of item 549, further comprising a first coating having a first composition and the second coating having a second composition.

688. The medical device of item 549, further comprising a first coating having a first composition and the second coating having a second composition, wherein the first composition and the second composition are different.

689. The medical device of item 549, further comprising a polymer.

690. The medical device of item 549, further comprising a polymeric carrier.

691. The medical device of item 549, further comprising a polymeric carrier, wherein the polymeric carrier comprises a copolymer.

692. The medical device of item 549, further comprising a polymeric carrier, wherein the polymeric carrier comprises a block copolymer.

693. The medical device of item 549, further comprising a polymeric carrier, wherein the polymeric carrier comprises a random copolymer.

694. The medical device of item 549, further comprising a polymeric carrier, wherein the polymeric carrier comprises a biodegradable polymer.

695. The medical device of item 549, further comprising a polymeric carrier, wherein the polymeric carrier comprises a non-biodegradable polymer.

696. The medical device of item 549, further comprising a polymeric carrier, wherein the polymeric carrier comprises a hydrophilic polymer.

697. The medical device of item 549, further comprising a polymeric carrier, wherein the polymeric carrier comprises a hydrophobic polymer.

698. The medical device of item 549, further comprising a polymeric carrier, wherein the polymeric carrier comprises a polymer having hydrophilic domains.

699. The medical device of item 549, further comprising a polymeric carrier, wherein the polymeric carrier comprises a polymer having hydrophobic domains.

700. The medical device of item 549, further comprising a polymeric carrier, wherein the polymeric carrier comprises a non-conductive polymer.

701. The medical device of item 549, further comprising a polymeric carrier, wherein the polymeric carrier comprises an elastomer.

702. The medical device of item 549, further comprising a polymeric carrier, wherein the polymeric carrier comprises a hydrogel.

703. The medical device of item 549, further comprising a polymeric carrier, wherein the polymeric carrier comprises a silicone polymer.

704. The medical device of item 549, further comprising a polymeric carrier, wherein the polymeric carrier comprises a hydrocarbon polymer.

705. The medical device of item 549, further comprising a polymeric carrier, wherein the polymeric carrier comprises a styrene-derived polymer.

706. The medical device of item 549, further comprising a polymeric carrier, wherein the polymeric carrier comprises a butadiene polymer.

707. The medical device of item 549, further comprising a polymeric carrier, wherein the polymeric carrier comprises a macromer.

708. The medical device of item 549, further comprising a polymeric carrier, wherein the polymeric carrier comprises a poly(ethylene glycol) polymer.

709. The medical device of item 549, further comprising a polymeric carrier, wherein the polymeric carrier comprises an amorphous polymer.

710. The medical device of item 549, further comprising a lubricious coating.

711. The medical device of item 549 wherein the anti-scarring agent is located within pores or holes of the electrical device.

712. The medical device of item 549 wherein the anti-scarring agent is located within a channel, lumen, or divet of the electrical device.

713. The medical device of item 549, further comprising a second pharmaceutically active agent.

714. The medical device of item 549, further comprising an anti-inflammatory agent.

715. The medical device of item 549, further comprising an agent that inhibits infection.

716. The medical device of item 549, further comprising an agent that inhibits infection, wherein the agent is an anthracycline.

717. The medical device of item 549, further comprising an agent that inhibits infection, wherein the agent is doxorubicin.

718. The medical device of item 549, further comprising an agent that inhibits infection, wherein the agent is mitoxantrone.

719. The medical device of item 549, further comprising an agent that inhibits infection, wherein the agent is a fluoropyrimidine.

720. The medical device of item 549, further comprising an agent that inhibits infection, wherein the agent is 5-fluorouracil (5-FU).

721. The medical device of item 549, further comprising an agent that inhibits infection, wherein the agent is a folic acid antagonist.

722. The medical device of item 549, further comprising an agent that inhibits infection, wherein the agent is methotrexate.

723. The medical device of item 549, further comprising an agent that inhibits infection, wherein the agent is a podophylotoxin.

724. The medical device of item 549, further comprising an agent that inhibits infection, wherein the agent is etoposide.

725. The medical device of item 549, further comprising an agent that inhibits infection, wherein the agent is a camptothecin.

726. The medical device of item 549, further comprising an agent that inhibits infection, wherein the agent is a hydroxyurea.

727. The medical device of item 549, further comprising an agent that inhibits infection, wherein the agent is a platinum complex.

728. The medical device of item 549, further comprising an agent that inhibits infection, wherein the agent is cisplatin.

729. The medical device of item 549, further comprising an anti-thrombotic agent.

730. The medical device of item 549, further comprising a visualization agent.

731. The medical device of item 549, further comprising a visualization agent, wherein the visualization agent is a radiopaque material, wherein the radiopaque material comprises a metal, a halogenated compound, or a barium containing compound.

732. The medical device of item 549, further comprising a visualization agent, wherein the visualization agent is a radiopaque material, wherein the radiopaque material comprises barium, tantalum, or technetium.

733. The medical device of item 549, further comprising a visualization agent, wherein the visualization agent is a MRI responsive material.

734. The medical device of item 549, further comprising a visualization agent, wherein the visualization agent comprises a gadolinium chelate.

735. The medical device of item 549, further comprising a visualization agent, wherein the visualization agent comprises iron, magnesium, manganese, copper, or chromium.

736. The medical device of item 549, further comprising a visualization agent, wherein the visualization agent comprises an iron oxide compound.

737. The medical device of item 549, further comprising a visualization agent, wherein the visualization agent comprises a dye, pigment, or colorant.

738. The medical device of item 549, further comprising an echogenic material.

739. The medical device of item 549, further comprising an echogenic material, wherein the echogenic material is in the form of a coating.

740. The medical device of item 549 wherein the device is sterile.

741. The medical device of item 549 wherein the anti-scarring agent inhibits adhesion between the medical device and a host into which the medical device is implanted.

742. The medical device of item 549 wherein the medical device delivers the anti-scarring agent locally to tissue proximate to the medical device.

743. The medical device of item 549 wherein the anti-scarring agent is released into tissue in the vicinity of the medical device after deployment of the medical device.

744. The medical device of item 549 wherein the anti-scarring agent is released into tissue in the vicinity of the medical device after deployment of the medical device, wherein the tissue is connective tissue.

745. The medical device of item 549 wherein the anti-scarring agent is released into tissue in the vicinity of the medical device after deployment of the medical device, wherein the tissue is muscle tissue.

746. The medical device of item 549 wherein the anti-scarring agent is released into tissue in the vicinity of the medical device after deployment of the medical device, wherein the tissue is nerve tissue.

747. The medical device of item 549 wherein the anti-scarring agent is released into tissue in the vicinity of the medical device after deployment of the medical device, wherein the tissue is epithelium tissue.

748. The medical device of item 549 wherein the anti-scarring agent is released in effective concentrations from the medical device over a period ranging from the time of deployment of the medical device to about 1 year.

749. The medical device of item 549 wherein the anti-scarring agent is released in effective concentrations from the medical device over a period ranging from about 1 month to 6 months.

750. The medical device of item 549 wherein the anti-scarring agent is released in effective concentrations from the medical device over a period ranging from about 1-90 days.

751. The medical device of item 549 wherein the anti-scarring agent is released in effective concentrations from the medical device at a constant rate.

752. The medical device of item 549 wherein the anti-scarring agent is released in effective concentrations from the medical device at an increasing rate.

753. The medical device of item 549 wherein the anti-scarring agent is released in effective concentrations from the medical device at a decreasing rate.

754. The medical device of item 549 wherein the anti-scarring agent is released in effective concentrations from the composition comprising the anti-scarring agent by diffusion over a period ranging from the time of deployment of the medical device to about 90 days.

755. The medical device of item 549 wherein the anti-scarring agent is released in effective concentrations from the composition comprising the anti-scarring agent by erosion of the composition over a period ranging from the time of deployment of the medical device to about 90 days.

756. The medical device of item 549 wherein the device comprises about 0.01 μg to about 10 μg of the anti-scarring agent.

757. The medical device of item 549 wherein the device comprises about 10 μg to about 10 mg of the anti-scarring agent.

758. The medical device of item 549 wherein the device comprises about 10 mg to about 250 mg of the anti-scarring agent.

759. The medical device of item 549 wherein the device comprises about 250 mg to about 1000 mg of the anti-scarring agent.

760. The medical device of item 549 wherein the device comprises about 1000 mg to about 2500 mg of the anti-scarring agent.

761. The medical device of item 549 wherein a surface of the device comprises less than 0.01 μg of the anti-scarring agent per mm² of device surface to which the anti-scarring agent is applied.

762. The medical device of item 549 wherein a surface of the device comprises about 0.01 μg to about 1 μg of the anti-scarring agent per mm² of device surface to which the anti-scarring agent is applied.

763. The medical device of item 549 wherein a surface of the device comprises about 1 μg to about 10 μg of the anti-scarring agent per mm² of device surface to which the anti-scarring agent is applied.

764. The medical device of item 549 wherein a surface of the device comprises about 10 μg to about 250 μg of the anti-scarring agent per mm² of device surface to which the anti-scarring agent is applied.

765. The medical device of item 549 wherein a surface of the device comprises about 250 μg to about 1000 μg of the anti-scarring agent of anti-scarring agent per mm² of device surface to which the anti-scarring agent is applied.

766. The medical device of item 549 wherein a surface of the device comprises about 1000 μg to about 2500 μg of the anti-scarring agent per mm² of device surface to which the anti-scarring agent is applied.

767. The medical device of item 549 wherein the agent or the composition is affixed to the electrical device.

768. The medical device of item 549 wherein the agent or the composition is covalently attached to the electrical device.

769. The medical device of item 549 wherein the agent or the composition is non-covalently attached to the electrical device.

770. The medical device of item 549 further comprising a coating that absorbs the agent or the composition.

771. The medical device of item 549 wherein the electrical device is interweaved with a thread composed of, or coated with, the agent or the composition.

772. The medical device of item 549 wherein a portion of the electrical device is covered with a sleeve that contains the agent or the composition.

773. The medical device of item 549 wherein the electrical device is completely covered with a sleeve that contains the agent or the composition.

774. The medical device of item 549 wherein a portion of the electrical device is covered with a mesh that contains the agent or the composition.

775. The medical device of item 549 wherein the electrical device is completely covered with a mesh that contains the agent or the composition.

776. The medical device of any one of items 549-775 wherein the neurostimulator comprises an intracranially implantable electrical control module and an electrode.

777. The medical device of any one of items 549-775 wherein the neurostimulator comprises a sensor and an electrode

778. A medical device, comprising a vagal nerve stimulator for treating epilepsy (i.e., an electrical device) and an anti-scarring agent or a composition comprising an anti-scarring agent, wherein the agent inhibits scarring between the medical device and the host into which the medical device is implanted.

779. The medical device of item 778 wherein the agent is an adensosine A2A receptor antagonist.

780. The medical device of item 778 wherein the agent is an AKT inhibitor.

781. The medical device of item 778 wherein the agent is an alpha 2 integrin antagonist, wherein the alpha 2 integrin antagonist is Pharmaprojects No. 5754 (Merck KgaA).

782. The medical device of item 778 wherein the agent is an alpha 4 integrin antagonist.

783. The medical device of item 778 wherein the agent is an alpha 7 nicotinic receptor agonist.

784. The medical device of item 778 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Aftenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

785. The medical device of item 778 wherein the agent is an apoptosis antagonist.

786. The medical device of item 778 wherein the agent is an apoptosis activator.

787. The medical device of item 778 wherein the agent is a beta 1 integrin antagonist.

788. The medical device of item 778 wherein the agent is a beta tubulin inhibitor.

789. The medical device of item 778 wherein the agent is a blocker of enzyme production in Hepatitis C.

790. The medical device of item 778 wherein the agent is a Bruton's tyrosine kinase inhibitor.

791. The medical device of item 778 wherein the agent is a calcineurin inhibitor.

792. The medical device of item 778 wherein the agent is a caspase 3 inhibitor.

793. The medical device of item 778 wherein the agent is a CC chemokine receptor antagonist.

794. The medical device of item 778 wherein the agent is a cell cycle inhibitor selected from the group consisting of SNS-595 (Sunesis), synthadotin, KRX-0403, and an analogue or derivative thereof.

795. The medical device of item 778 wherein the agent is a cathepsin B inhibitor.

796. The medical device of item 778 wherein the agent is a cathepsin K inhibitor, wherein the cathepsin K inhibitor is 462795 (GlaxoSmithKline), INPL-022-D6 (Amura Therapeutics), or an analogue or derivative thereof.

797. The medical device of item 778 wherein the agent is a cathepsin L inhibitor.

798. The medical device of item 778 wherein the agent is a CD40 antagonist.

799. The medical device of item 778 wherein the agent is a chemokine receptor agonist.

800. The medical device of item 778 wherein the agent is a chymase inhibitor.

801. The medical device of item 778 wherein the agent is a collagenase antagonist.

802. The medical device of item 778 wherein the agent is a CXCR antagonist.

803. The medical device of item 778 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAKI inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

804. The medical device of item 778 wherein the agent is a cyclooxygenase 1 inhibitor.

805. The medical device of item 778 wherein the agent is a DHFR inhibitor.

806. The medical device of item 778 wherein the agent is a dual integrin inhibitor.

807. The medical device of item 778 wherein the agent is an elastase inhibitor.

808. The medical device of item 778 wherein the agent is an elongation factor-1 alpha inhibitor.

809. The medical device of item 778 wherein the agent is an endothelial growth factor antagonist.

810. The medical device of item 778 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

811. The medical device of item 778 wherein the agent is an endotoxin antagonist.

812. The medical device of item 778 wherein the agent is an epothilone and tubulin binder.

813. The medical device of item 778 wherein the agent is an estrogen receptor antagonist.

814. The medical device of item 778 wherein the agent is an FGF inhibitor.

815. The medical device of item 778 wherein the agent is a farnexyl transferase inhibitor.

816. The medical device of item 778 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

817. The medical device of item 778 wherein the agent is an FLT-3 kinase inhibitor.

818. The medical device of item 778 wherein the agent is an FGF receptor kinase inhibitor.

819. The medical device of item 778 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

820. The medical device of item 778 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy:1,4-dihydro-5′-(2-methylpropyl)-1-oxo-), and an analogue or derivative thereof.

821. The medical device of item 778 wherein the agent is a histone deacetylase inhibitor.

822. The medical device of item 778 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

823. The medical device of item 778 wherein the agent is an ICAM inhibitor.

824. The medical device of item 778 wherein the agent is an IL, ICE and IRAK antagonist, wherein the antagonist is a CJ-14877, CP-424174 (Pfizer), NF-61 (Negma-Lerads), and an analogue or derivative thereof.

825. The medical device of item 778 wherein the agent is an IL-2 inhibitor.

826. The medical device of item 778 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

827. The medical device of item 778 wherein the agent is an IMPDH (inosine monophosphate).

828. The medical device of item 778 wherein the agent is an integrin antagonist.

829. The medical device of item 778 wherein the agent is an interleukin antagonist.

830. The medical device of item 778 wherein the agent is an inhibitor of type III receptor tyrosine kinase.

831. The medical device of item 778 wherein the agent is an irreversible inhibitor of enzyme methionine aminopeptidase type 2.

832. The medical device of item 778 wherein the agent is an isozyme selective delta protein kinase C inhibitor.

833. The medical device of item 778 wherein the agent a JAK3 enzyme inhibitor.

834. The medical device of item 778 wherein the agent is a JNK inhibitor.

835. The medical device of item 778 wherein the agent is a kinase inhibitor.

836. The medical device of item 778 wherein the agent is kinesin antagonist.

837. The medical device of item 778 wherein the agent is a kinesin antagonist.

838. The medical device of item 778 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

839. The medical device of item 778 wherein the agent is an MAP kinase inhibitor.

840. The medical device of item 778 wherein the agent is a matrix metalloproteinase inhibitor.

841. The medical device of item 778 wherein the agent is an MCP-CCR2 inhibitor.

842. The medical device of item 778 wherein the agent is an mTOR inhibitor.

843. The medical device of item 778 wherein the agent is an mTOR kinase inhibitor.

844. The medical device of item 778 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

845. The medical device of item 778 wherein the agent is an MIF inhibitor.

846. The medical device of item 778 wherein the agent is an MMP inhibitor.

847. The medical device of item 778 wherein the agent is a neurokinin (NK) antagonist selected from the group consisting of anthrotainin (CAS No. 14808440-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

848. The medical device of item 778 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

849. The medical device of item 778 wherein the agent is a nitric oxide agonist.

850. The medical device of item 778 wherein the agent is an ornithine decarboxylase inhibitor.

851. The medical device of item 778 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

852. The medical device of item 778 wherein the agent is a palmitoyl-protein thioesterase inhibitor.

853. The medical device of item 778 wherein the agent is a PDGF receptor kinase inhibitor selected from the group consisting of ML-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

854. The medical device of item 778 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

855. The medical device of item 778 wherein the agent is a phosphatase inhibitor.

856. The medical device of item 778 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

857. The medical device of item 778 wherein the agent is a PKC inhibitor.

858. The medical device of item 778 wherein the agent is a platelet activating factor antagonist.

859. The medical device of item 778 wherein the agent is a platelet-derived growth factor receptor kinase inhibitor.

860. The medical device of item 778 wherein the agent is a prolyl hydroxylase inhibitor.

861. The medical device of item 778 wherein the agent is a polymorphonuclear neutrophil inhibitor.

862. The medical device of item 778 wherein the agent is a protein kinase B inhibitor.

863. The medical device of item 778 wherein the agent is a protein kinase C stimulant.

864. The medical device of item 778 wherein the agent is a purine nucleoside analogue.

865. The medical device of item 778 wherein the agent is a purinoreceptor P2X antagonist.

866. The medical device of item 778 wherein the agent is a Raf kinase inhibitor.

867. The medical device of item 778 wherein the agent is a reversible inhibitor of ErbB1 and ErbB2.

868. The medical device of item 778 wherein the agent is a ribonucleoside triphosphate reductase inhibitor.

869. The medical device of item 778 wherein the agent is an SDF-1 antagonist.

870. The medical device of item 778 wherein the agent is a sheddase inhibitor.

871. The medical device of item 778 wherein the agent is an SRC inhibitor.

872. The medical device of item 778 wherein the agent is a stromelysin inhibitor.

873. The medical device of item 778 wherein the agent is an Syk kinase inhibitor.

874. The medical device of item 778 wherein the agent is a telomerase inhibitor.

875. The medical device of item 778 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

876. The medical device of item 778 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

877. The medical device of item 778 wherein the agent is a Toll receptor inhibitor.

878. The medical device of item 778 wherein the agent is a tubulin antagonist.

879. The medical device of item 778 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), C P-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (051 Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-21neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (1 mClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (GAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

880. The medical device of item 778 wherein the agent is a VEGF inhibitor

881. The medical device of item 778 wherein the agent is a vitamin D receptor agonist.

882. The medical device of item 778 wherein the agent is ZD-6474 (an angiogenesis inhibitor).

883. The medical device of item 778 wherein the agent is AP-23573 (an mTOR inhibitor).

884. The medical device of item 778 wherein the agent is synthadotin (a tubulin antagonist).

885. The medical device of item 778 wherein the agent is S-0885 (a collagenase inhibitor).

886. The medical device of item 778 wherein the agent is aplidine (an elongation factor-1 alpha inhibitor).

887. The medical device of item 778 wherein the agent is ixabepilone (an epithilone).

888. The medical device of item 778 wherein the agent is IDN-5390 (an angiogenesis inhibitor).

889. The medical device of item 778 wherein the agent is SB-2723005 (an angiogenesis inhibitor).

890. The medical device of item 778 wherein the agent is ABT-518 (an angiogenesis inhibitor).

891. The medical device of item 778 wherein the agent is combretastatin (an angiogenesis inhibitor).

892. The medical device of item 778 wherein the agent is anecortave acetate (an angiogenesis inhibitor).

893. The medical device of item 778 wherein the agent is SB-715992 (a kinesin antagonist).

894. The medical device of item 778 wherein the agent is temsirolimus (an mTOR inhibitor).

895. The medical device of item 778 wherein the agent is adalimumab (a TNFα antagonist).

896. The medical device of item 778, further comprising a coating, wherein the coating comprises the anti-scarring agent and a polymer.

897. The medical device of item 778, further comprising a coating, wherein the coating comprises the anti-scarring agent.

898. The medical device of item 778, further comprising a coating, wherein the coating is disposed on a surface of the electrical device.

899. The medical device of item 778, further comprising a coating, wherein the coating directly contacts the electrical device

900. The medical device of item 778, further comprising a coating, wherein the coating indirectly contacts the electrical device.

901. The medical device of item 778, further comprising a coating, wherein the coating partially covers the electrical device.

902. The medical device of item 778, further comprising a coating, wherein the coating completely covers the electrical device.

903. The medical device of item 778, further comprising a coating, wherein the coating is a uniform coating.

904. The medical device of item 778, further comprising a coating, wherein the coating is a non-uniform coating.

905. The medical device of item 778, further comprising a coating, wherein the coating is a discontinuous coating.

906. The medical device of item 778, further comprising a coating, wherein the coating is a patterned coating.

907. The medical device of item 778, further comprising a coating, wherein the coating has a thickness of 100 μm or less.

908. The medical device of item 778, further comprising a coating, wherein the coating has a thickness of 10 μm or less.

909. The medical device of item 778, further comprising a coating, wherein the coating adheres to the surface of the electrical device upon deployment of the medical device.

910. The medical device of item 778, further comprising a coating, wherein the coating is stable at room temperature for a period of 1 year.

911. The medical device of item 778, further comprising a coating, wherein the anti-scarring agent is present in the coating in an amount ranging between about 0.0001% to about 1% by weight.

912. The medical device of item 778, further comprising a coating, wherein the anti-scarring agent is present in the coating in an amount ranging between about 1% to about 10% by weight.

913. The medical device of item 778, further comprising a coating, wherein the anti-scarring agent is present in the coating in an amount ranging between about 10% to about 25% by weight.

914. The medical device of item 778, further comprising a coating, wherein the anti-scarring agent is present in the coating in an amount ranging between about 25% to about 70% by weight.

915. The medical device of item 778, further comprising a coating, wherein the coating further comprises a polymer.

916. The medical device of item 778, further comprising a first coating having a first composition and the second coating having a second composition.

917. The medical device of item 778, further comprising a first coating having a first composition and the second coating having a second composition, wherein the first composition and the second composition are different.

918. The medical device of item 778, further comprising a polymer.

919. The medical device of item 778, further comprising a polymeric carrier.

920. The medical device of item 778, further comprising a polymeric carrier, wherein the polymeric carrier comprises a copolymer.

921. The medical device of item 778, further comprising a polymeric carrier, wherein the polymeric carrier comprises a block copolymer.

922. The medical device of item 778, further comprising a polymeric carrier, wherein the polymeric carrier comprises a random copolymer.

923. The medical device of item 778, further comprising a polymeric carrier, wherein the polymeric carrier comprises a biodegradable polymer.

924. The medical device of item 778, further comprising a polymeric carrier, wherein the polymeric carrier comprises a non-biodegradable polymer.

925. The medical device of item 778, further comprising a polymeric carrier, wherein the polymeric carrier comprises a hydrophilic polymer.

926. The medical device of item 778, further comprising a polymeric carrier, wherein the polymeric carrier comprises a hydrophobic polymer.

927. The medical device of item 778, further comprising a polymeric carrier, wherein the polymeric carrier comprises a polymer having hydrophilic domains.

928. The medical device of item 778, further comprising a polymeric carrier, wherein the polymeric carrier comprises a polymer having hydrophobic domains.

929. The medical device of item 778, further comprising a polymeric carrier, wherein the polymeric carrier comprises a non-conductive polymer.

930. The medical device of item 778, further comprising a polymeric carrier, wherein the polymeric carrier comprises an elastomer.

931. The medical device of item 778, further comprising a polymeric carrier, wherein the polymeric carrier comprises a hydrogel.

932. The medical device of item 778, further comprising a polymeric carrier, wherein the polymeric carrier comprises a silicone polymer.

933. The medical device of item 778, further comprising a polymeric carrier, wherein the polymeric carrier comprises a hydrocarbon polymer.

934. The medical device of item 778, further comprising a polymeric carrier, wherein the polymeric carrier comprises a styrene-derived polymer.

935. The medical device of item 778, further comprising a polymeric carrier, wherein the polymeric carrier comprises a butadiene polymer.

936. The medical device of item 778, further comprising a polymeric carrier, wherein the polymeric carrier comprises a macromer.

937. The medical device of item 778, further comprising a polymeric carrier, wherein the polymeric carrier comprises a poly(ethylene glycol) polymer.

938. The medical device of item 778, further comprising a polymeric carrier, wherein the polymeric carrier comprises an amorphous polymer.

939. The medical device of item 778, further comprising a lubricious coating.

940. The medical device of item 778 wherein the anti-scarring agent is located within pores or holes of the electrical device

941. The medical device of item 778 wherein the anti-scarring agent is located within a channel, lumen, or divet of the electrical device.

942. The medical device of item 778, further comprising a second pharmaceutically active agent.

943. The medical device of item 778, further comprising an anti-inflammatory agent.

944. The medical device of item 778, further comprising an agent that inhibits infection.

945. The medical device of item 778, further comprising an agent that inhibits infection, wherein the agent is an anthracycline.

946. The medical device of item 778, further comprising an agent that inhibits infection, wherein the agent is doxorubicin.

947. The medical device of item 778, further comprising an agent that inhibits infection, wherein the agent is mitoxantrone.

948. The medical device of item 778, further comprising an agent that inhibits infection, wherein the agent is a fluoropyrimidine.

949. The medical device of item 778, further comprising an agent that inhibits infection, wherein the agent is 5-fluorouracil (5-FU).

950. The medical device of item 778, further comprising an agent that inhibits infection, wherein the agent is a folic acid antagonist.

951. The medical device of item 778, further comprising an agent that inhibits infection, wherein the agent is methotrexate.

952. The medical device of item 778, further comprising an agent that inhibits infection, wherein the agent is a podophylotoxin.

953. The medical device of item 778, further comprising an agent that inhibits infection, wherein the agent is etoposide.

954. The medical device of item 778, further comprising an agent that inhibits infection, wherein the agent is a camptothecin.

955. The medical device of item 778, further comprising an agent that inhibits infection, wherein the agent is a hydroxyurea.

956. The medical device of item 778, further comprising an agent that inhibits infection, wherein the agent is a platinum complex.

957. The medical device of item 778, further comprising an agent that inhibits infection, wherein the agent is cisplatin.

958. The medical device of item 778, further comprising an anti-thrombotic agent.

959. The medical device of item 778, further comprising a visualization agent.

960. The medical device of item 778, further comprising a visualization agent, wherein the visualization agent is a radiopaque material, wherein the radiopaque material comprises a metal, a halogenated compound, or a barium containing compound.

961. The medical device of item 778, further comprising a visualization agent, wherein the visualization agent is a radiopaque material, wherein the radiopaque material comprises barium, tantalum, or technetium.

962. The medical device of item 778, further comprising a visualization agent, wherein the visualization agent is a MRI responsive material.

963. The medical device of item 778, further comprising a visualization agent, wherein the visualization agent comprises a gadolinium chelate.

964. The medical device of item 778, further comprising a visualization agent, wherein the visualization agent comprises iron, magnesium, manganese, copper, or chromium.

965. The medical device of item 778, further comprising a visualization agent, wherein the visualization agent comprises an iron oxide compound.

966. The medical device of item 778, further comprising a visualization agent, wherein the visualization agent comprises a dye, pigment, or colorant.

967. The medical device of item 778, further comprising an echogenic material.

968. The medical device of item 778, further comprising an echogenic material, wherein the echogenic material is in the form of a coating.

969. The medical device of item 778 wherein the device is sterile.

970. The medical device of item 778 wherein the anti-scarring agent inhibits adhesion between the medical device and a host into which the medical device is implanted.

971. The medical device of item 778 wherein the medical device delivers the anti-scarring agent locally to tissue proximate to the medical device.

972. The medical device of item 778 wherein the anti-scarring agent is released into tissue in the vicinity of the medical device after deployment of the medical device.

973. The medical device of item 778 wherein the anti-scarring agent is released into tissue in the vicinity of the medical device after deployment of the medical device, wherein the tissue is connective tissue.

974. The medical device of item 778 wherein the anti-scarring agent is released into tissue in the vicinity of the medical device after deployment of the medical device, wherein the tissue is muscle tissue.

975. The medical device of item 778 wherein the anti-scarring agent is released into tissue in the vicinity of the medical device after deployment of the medical device, wherein the tissue is nerve tissue.

976. The medical device of item 778 wherein the anti-scarring agent is released into tissue in the vicinity of the medical device after deployment of the medical device, wherein the tissue is epithelium tissue.

977. The medical device of item 778 wherein the anti-scarring agent is released in effective concentrations from the medical device over a period ranging from the time of deployment of the medical device to about 1 year.

978. The medical device of item 778 wherein the anti-scarring agent is released in effective concentrations from the medical device over a period ranging from about 1 month to 6 months.

979. The medical device of item 778 wherein the anti-scarring agent is released in effective concentrations from the medical device over a period ranging from about 1-90 days.

980. The medical device of item 778 wherein the anti-scarring agent is released in effective concentrations from the medical device at a constant rate.

981. The medical device of item 778 wherein the anti-scarring agent is released in effective concentrations from the medical device at an increasing rate.

982. The medical device of item 778 wherein the anti-scarring agent is released in effective concentrations from the medical device at a decreasing rate.

983. The medical device of item 778 wherein the anti-scarring agent is released in effective concentrations from the composition comprising the anti-scarring agent by diffusion over a period ranging from the time of deployment of the medical device to about 90 days.

984. The medical device of item 778 wherein the anti-scarring agent is released in effective concentrations from the composition comprising the anti-scarring agent by erosion of the composition over a period ranging from the time of deployment of the medical device to about 90 days.

985. The medical device of item 778 wherein the device comprises about 0.01 μg to about 10 μg of the anti-scarring agent.

986. The medical device of item 778 wherein the device comprises about 10 μg to about 10 mg of the anti-scarring agent.

987. The medical device of item 778 wherein the device comprises about 10 mg to about 250 mg of the anti-scarring agent.

988. The medical device of item 778 wherein the device comprises about 250 mg to about 1000 mg of the anti-scarring agent.

989. The medical device of item 778 wherein the device comprises about 1000 mg to about 2500 mg of the anti-scarring agent.

990. The medical device of item 778 wherein a surface of the device comprises less than 0.01 μg of the anti-scarring agent per mm² of device surface to which the anti-scarring agent is applied.

991. The medical device of item 778 wherein a surface of the device comprises about 0.01 μg to about 1 μg of the anti-scarring agent per mm² of device surface to which the anti-scarring agent is applied.

992. The medical device of item 778 wherein a surface of the device comprises about 1 μg to about 10 μg of the anti-scarring agent per mm² of device surface to which the anti-scarring agent is applied.

993. The medical device of item 778 wherein a surface of the device comprises about 10 μg to about 250 μg of the anti-scarring agent per mm² of device surface to which the anti-scarring agent is applied.

994. The medical device of item 778 wherein a surface of the device comprises about 250 μg to about 1000 μg of the anti-scarring agent of anti-scarring agent per mm² of device surface to which the anti-scarring agent is applied.

995. The medical device of item 778 wherein a surface of the device comprises about 1000 μg to about 2500 μg of the anti-scarring agent per mm² of device surface to which the anti-scarring agent is applied.

996. The medical device of item 778 wherein the agent or the composition is affixed to the electrical device.

997. The medical device of item 778 wherein the agent or the composition is covalently attached to the electrical device.

998. The medical device of item 778 wherein the agent or the composition is non-covalently attached to the electrical device.

999. The medical device of item 778 further comprising a coating that absorbs the agent or the composition.

1000. The medical device of item 778 wherein the electrical device is interweaved with a thread composed of, or coated with, the agent or the composition.

1001. The medical device of item 778 wherein a portion of the electrical device is covered with a sleeve that contains the agent or the composition.

1002. The medical device of item 778 wherein the electrical device is completely covered with a sleeve that contains the agent or the composition.

1003. The medical device of item 778 wherein a portion of the electrical device is covered with a mesh that contains the agent or the composition.

1004. The medical device of item 778 wherein the electrical device is completely covered with a mesh that contains the agent or the composition.

1005. A medical device, comprising a vagal nerve stimulator (i.e., an electrical device) and an anti-scarring agent or a composition comprising an anti-scarring agent, wherein the agent inhibits scarring between the medical device and the host into which the medical device is implanted.

1006. The medical device of item 1005 wherein the agent is an adensosine A2A receptor antagonist.

1007. The medical device of item 1005 wherein the agent is an AKT inhibitor.

1008. The medical device of item 1005 wherein the agent is an alpha 2 integrin antagonist, wherein the alpha 2 integrin antagonist is Pharmaprojects No. 5754 (Merck KgaA).

1009. The medical device of item 1005 wherein the agent is an alpha 4 integrin antagonist.

1010. The medical device of item 1005 wherein the agent is an alpha 7 nicotinic receptor agonist.

1011. The medical device of item 1005 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

1012. The medical device of item 1005 wherein the agent is an apoptosis antagonist.

1013. The medical device of item 1005 wherein the agent is an apoptosis activator

1014. The medical device of item 1005 wherein the agent is a beta 1 integrin antagonist.

1015. The medical device of item 1005 wherein the agent is a beta tubulin inhibitor.

1016. The medical device of item 1005 wherein the agent is a blocker of enzyme production in Hepatitis C.

1017. The medical device of item 1005 wherein the agent is a Bruton's tyrosine kinase inhibitor.

1018. The medical device of item 1005 wherein the agent is a calcineurin inhibitor.

1019. The medical device of item 1005 wherein the agent is a caspase 3 inhibitor.

1020. The medical device of item 1005 wherein the agent is a CC chemokine receptor antagonist.

1021. The medical device of item 1005 wherein the agent is a cell cycle inhibitor selected from the group consisting of SNS-595 (Sunesis), synthadotin, KRX-0403, and an analogue or derivative thereof.

1022. The medical device of item 1005 wherein the agent is a cathepsin B inhibitor.

1023. The medical device of item 1005 wherein the agent is a cathepsin K inhibitor, wherein the cathepsin K inhibitor is 462795 (GlaxoSmithKline), INPL-022-D6 (Amura Therapeutics), or an analogue or derivative thereof.

1024. The medical device of item 1005 wherein the agent is a cathepsin L inhibitor.

1025. The medical device of item 1005 wherein the agent is a CD40 antagonist.

1026. The medical device of item 1005 wherein the agent is a chemokine receptor agonist.

1027. The medical device of item 1005 wherein the agent is a chymase inhibitor.

1028. The medical device of item 1005 wherein the agent is a collagenase antagonist.

1029. The medical device of item 1005 wherein the agent is a CXCR antagonist.

1030. The medical device of item 1005 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

1031. The medical device of item 1005 wherein the agent is a cyclooxygenase 1 inhibitor.

1032. The medical device of item 1005 wherein the agent is a DHFR inhibitor.

1033. The medical device of item 1005 wherein the agent is a dual integrin inhibitor.

1034. The medical device of item 1005 wherein the agent is an elastase inhibitor.

1035. The medical device of item 1005 wherein the agent is an elongation factor-1 alpha inhibitor.

1036. The medical device of item 1005 wherein the agent is an endothelial growth factor antagonist.

1037. The medical device of item 1005 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

1038. The medical device of item 1005 wherein the agent is an endotoxin antagonist.

1039. The medical device of item 1005 wherein the agent is an epothilone and tubulin binder.

1040. The medical device of item 1005 wherein the agent is an estrogen receptor antagonist.

1041. The medical device of item 1005 wherein the agent is an FGF inhibitor.

1042. The medical device of item 1005 wherein the agent is a farnexyl transferase inhibitor.

1043. The medical device of item 1005 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

1044. The medical device of item 1005 wherein the agent is an FLT-3 kinase inhibitor.

1045. The medical device of item 1005 wherein the agent is an FGF receptor kinase inhibitor.

1046. The medical device of item 1005 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

1047. The medical device of item 1005 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

1048. The medical device of item 1005 wherein the agent is a histone deacetylase inhibitor.

1049. The medical device of item 1005 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

1050. The medical device of item 1005 wherein the agent is an ICAM inhibitor.

1051. The medical device of item 1005 wherein the agent is an IL, ICE and IRAK antagonist, wherein the antagonist is a CJ-14877, CP-424174 (Pfizer), NF-61 (Negma-Lerads), and an analogue or derivative thereof.

1052. The medical device of item 1005 wherein the agent is an IL-2 inhibitor.

1053. The medical device of item 1005 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

1054. The medical device of item 1005 wherein the agent is an IMPDH (inosine monophosphate).

1055. The medical device of item 1005 wherein the agent is an integrin antagonist.

1056. The medical device of item 1005 wherein the agent is an interleukin antagonist.

1057. The medical device of item 1005 wherein the agent is an inhibitor of type III receptor tyrosine kinase.

1058. The medical device of item 1005 wherein the agent is an irreversible inhibitor of enzyme methionine aminopeptidase type 2.

1059. The medical device of item 1005 wherein the agent is an isozyme selective delta protein kinase C inhibitor.

1060. The medical device of item 1005 wherein the agent a JAK3 enzyme inhibitor.

1061. The medical device of item 1005 wherein the agent is a JNK inhibitor.

1062. The medical device of item 1005 wherein the agent is a kinase inhibitor.

1063. The medical device of item 1005 wherein the agent is kinesin antagonist.

1064. The medical device of item 1005 wherein the agent is a kinesin antagonist.

1065. The medical device of item 1005 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

1066. The medical device of item 1005 wherein the agent is an MAP kinase inhibitor.

1067. The medical device of item 1005 wherein the agent is a matrix metalloproteinase inhibitor.

1068. The medical device of item 1005 wherein the agent is an MCP-CCR2 inhibitor.

1069. The medical device of item 1005 wherein the agent is an mTOR inhibitor.

1070. The medical device of item 1005 wherein the agent is an mTOR kinase inhibitor.

1071. The medical device of item 1005 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

1072. The medical device of item 1005 wherein the agent is an MIF inhibitor.

1073. The medical device of item 1005 wherein the agent is an MMP inhibitor.

1074. The medical device of item 1005 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

1075. The medical device of item 1005 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

1076. The medical device of item 1005 wherein the agent is a nitric oxide agonist.

1077. The medical device of item 1005 wherein the agent is an ornithine decarboxylase inhibitor.

1078. The medical device of item 1005 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

1079. The medical device of item 1005 wherein the agent is a palmitoyl-protein thioesterase inhibitor.

1080. The medical device of item 1005 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

1081. The medical device of item 1005 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVAN DAMET, rosiglitazone maleate+metformin, AVANDAMET (GlaxoSmithKline), tesaglitazar (AstraZeneca), and an analogue or derivative thereof.

1082. The medical device of item 1005 wherein the agent is a phosphatase inhibitor.

1083. The medical device of item 1005 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

1084. The medical device of item 1005 wherein the agent is a PKC inhibitor.

1085. The medical device of item 1005 wherein the agent is a platelet activating factor antagonist.

1086. The medical device of item 1005 wherein the agent is a platelet-derived growth factor receptor kinase inhibitor.

1087. The medical device of item 1005 wherein the agent is a prolyl hydroxylase inhibitor.

1088. The medical device of item 1005 wherein the agent is a polymorphonuclear neutrophil inhibitor.

1089. The medical device of item 1005 wherein the agent is a protein kinase B inhibitor.

1090. The medical device of item 1005 wherein the agent is a protein kinase C stimulant.

1091. The medical device of item 1005 wherein the agent is a purine nucleoside analogue.

1092. The medical device of item 1005 wherein the agent is a purinoreceptor P2X antagonist.

1093. The medical device of item 1005 wherein the agent is a Raf kinase inhibitor.

1094. The medical device of item 1005 wherein the agent is a reversible inhibitor of ErbB1 and ErbB2.

1095. The medical device of item 1005 wherein the agent is a ribonucleoside triphosphate reductase inhibitor.

1096. The medical device of item 1005 wherein the agent is an SDF-1 antagonist.

1097. The medical device of item 1005 wherein the agent is a sheddase inhibitor.

1098. The medical device of item 1005 wherein the agent is an SRC inhibitor.

1099. The medical device of item 1005 wherein the agent is a stromelysin inhibitor.

1100. The medical device of item 1005 wherein the agent is an Syk kinase inhibitor.

1101. The medical device of item 1005 wherein the agent is a telomerase inhibitor.

1102. The medical device of item 1005 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

1103. The medical device of item 1005 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

1104. The medical device of item 1005 wherein the agent is a Toll receptor inhibitor.

1105. The medical device of item 1005 wherein the agent is a tubulin antagonist.

1106. The medical device of item 1005 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her21neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

1107. The medical device of item 1005 wherein the agent is a VEGF inhibitor.

1108. The medical device of item 1005 wherein the agent is a vitamin D receptor agonist.

1109. The medical device of item 1005 wherein the agent is ZD-6474 (an angiogenesis inhibitor).

1110. The medical device of item 1005 wherein the agent is AP-23573 (an mTOR inhibitor).

1111. The medical device of item 1005 wherein the agent is synthadotin (a tubulin antagonist).

1112. The medical device of item 1005 wherein the agent is S-0885 (a collagenase inhibitor).

1113. The medical device of item 1005 wherein the agent is aplidine (an elongation factor-1 alpha inhibitor).

1114. The medical device of item 1005 wherein the agent is ixabepilone (an epithilone).

1115. The medical device of item 1005 wherein the agent is IDN-5390 (an angiogenesis inhibitor).

1116. The medical device of item 1005 wherein the agent is SB-2723005 (an angiogenesis inhibitor).

1117. The medical device of item 1005 wherein the agent is ABT-518 (an angiogenesis inhibitor).

1118. The medical device of item 1005 wherein the agent is combretastatin (an angiogenesis inhibitor).

1119. The medical device of item 1005 wherein the agent is anecortave acetate (an angiogenesis inhibitor).

1120. The medical device of item 1005 wherein the agent is SB-715992 (a kinesin antagonist).

1121. The medical device of item 1005 wherein the agent is temsirolimus (an mTOR inhibitor).

1122. The medical device of item 1005 wherein the agent is adalimumab (a TNFα antagonist).

1123. The medical device of item 1005, further comprising a coating, wherein the coating comprises the anti-scarring agent and a polymer.

1124. The medical device of item 1005, further comprising a coating, wherein the coating comprises the anti-scarring agent.

1125. The medical device of item 1005, further comprising a coating, wherein the coating is disposed on a surface of the electrical device.

1126. The medical device of item 1005, further comprising a coating, wherein the coating directly contacts the electrical device.

1127. The medical device of item 1005, further comprising a coating, wherein the coating indirectly contacts the electrical device.

1128. The medical device of item 1005, further comprising a coating, wherein the coating partially covers the electrical device.

1129. The medical device of item 1005, further comprising a coating, wherein the coating completely covers the electrical device.

1130. The medical device of item 1005, further comprising a coating, wherein the coating is a uniform coating.

1131. The medical device of item 1005, further comprising a coating, wherein the coating is a non-uniform coating.

1132. The medical device of item 1005, further comprising a coating, wherein the coating is a discontinuous coating.

1133. The medical device of item 1005, further comprising a coating, wherein the coating is a patterned coating.

1134. The medical device of item 1005, further comprising a coating, wherein the coating has a thickness of 100 μm or less.

1135. The medical device of item 1005, further comprising a coating, wherein the coating has a thickness of 10 μm or less.

1136. The medical device of item 1005, further comprising a coating, wherein the coating adheres to the surface of the electrical device upon deployment of the medical device.

1137. The medical device of item 1005, further comprising a coating, wherein the coating is stable at room temperature for a period of 1 year.

1138. The medical device of item 1005, further comprising a coating, wherein the anti-scarring agent is present in the coating in an amount ranging between about 0.0001% to about 1% by weight.

1139. The medical device of item 1005, further comprising a coating, wherein the anti-scarring agent is present in the coating in an amount ranging between about 1% to about 10% by weight.

1140. The medical device of item 1005, further comprising a coating, wherein the anti-scarring agent is present in the coating in an amount ranging between about 10% to about 25% by weight.

1141. The medical device of item 1005, further comprising a coating, wherein the anti-scarring agent is present in the coating in an amount ranging between about 25% to about 70% by weight.

1142. The medical device of item 1005, further comprising a coating, wherein the coating further comprises a polymer

1143. The medical device of item 1005, further comprising a first coating having a first composition and the second coating having a second composition.

1144. The medical device of item 1005, further comprising a first coating having a first composition and the second coating having a second composition, wherein the first composition and the second composition are different.

1145. The medical device of item 1005, further comprising a polymer.

1146. The medical device of item 1005, further comprising a polymeric carrier.

1147. The medical device of item 1005, further comprising a polymeric carrier, wherein the polymeric carrier comprises a copolymer.

1148. The medical device of item 1005, further comprising a polymeric carrier, wherein the polymeric carrier comprises a block copolymer.

1149. The medical device of item 1005, further comprising a polymeric carrier, wherein the polymeric carrier comprises a random copolymer.

1150. The medical device of item 1005, further comprising a polymeric carrier, wherein the polymeric carrier comprises a biodegradable polymer.

1151. The medical device of item 1005, further comprising a polymeric carrier, wherein the polymeric carrier comprises a non-biodegradable polymer.

1152. The medical device of item 1005, further comprising a polymeric carrier, wherein the polymeric carrier comprises a hydrophilic polymer.

1153. The medical device of item 1005, further comprising a polymeric carrier, wherein the polymeric carrier comprises a hydrophobic polymer.

1154. The medical device of item 1005, further comprising a polymeric carrier, wherein the polymeric carrier comprises a polymer having hydrophilic domains.

1155. The medical device of item 1005, further comprising a polymeric carrier, wherein the polymeric carrier comprises a polymer having hydrophobic domains.

1156. The medical device of item 1005, further comprising a polymeric carrier, wherein the polymeric carrier comprises a non-conductive polymer.

1157. The medical device of item 1005, further comprising a polymeric carrier, wherein the polymeric carrier comprises an elastomer.

1158. The medical device of item 1005, further comprising a polymeric carrier, wherein the polymeric carrier comprises a hydrogel.

1159. The medical device of item 1005, further comprising a polymeric carrier, wherein the polymeric carrier comprises a silicone polymer.

1160. The medical device of item 1005, further comprising a polymeric carrier, wherein the polymeric carrier comprises a hydrocarbon polymer.

1161. The medical device of item 1005, further comprising a polymeric carrier, wherein the polymeric carrier comprises a styrene-derived polymer.

1162. The medical device of item 1005, further comprising a polymeric carrier, wherein the polymeric carrier comprises a butadiene polymer.

1163. The medical device of item 1005, further comprising a polymeric carrier, wherein the polymeric carrier comprises a macromer.

1164. The medical device of item 1005, further comprising a polymeric carrier, wherein the polymeric carrier comprises a poly(ethylene glycol) polymer.

1165. The medical device of item 1005, further comprising a polymeric carrier, wherein the polymeric carrier comprises an amorphous polymer.

1166. The medical device of item 1005, further comprising a lubricious coating.

1167. The medical device of item 1005 wherein the anti-scarring agent is located within pores or holes of the electrical device.

1168. The medical device of item 1005 wherein the anti-scarring agent is located within a channel, lumen, or divet of the electrical device.

1169. The medical device of item 1005, further comprising a second pharmaceutically active agent.

1170. The medical device of item 1005, further comprising an anti-inflammatory agent.

1171. The medical device of item 1005, further comprising an agent that inhibits infection.

1172. The medical device of item 1005, further comprising an agent that inhibits infection, wherein the agent is an anthracycline.

1173. The medical device of item 1005, further comprising an agent that inhibits infection, wherein the agent is doxorubicin.

1174. The medical device of item 1005, further comprising an agent that inhibits infection, wherein the agent is mitoxantrone.

1175. The medical device of item 1005, further comprising an agent that inhibits infection, wherein the agent is a fluoropyrimidine.

1176. The medical device of item 1005, further comprising an agent that inhibits infection, wherein the agent is 5-fluorouracil (5-FU).

1177. The medical device of item 1005, further comprising an agent that inhibits infection, wherein the agent is a folic acid antagonist.

1178. The medical device of item 1005, further comprising an agent that inhibits infection, wherein the agent is methotrexate.

1179. The medical device of item 1005, further comprising an agent that inhibits infection, wherein the agent is a podophylotoxin.

1180. The medical device of item 1005, further comprising an agent that inhibits infection, wherein the agent is etoposide.

1181. The medical device of item 1005, further comprising an agent that inhibits infection, wherein the agent is a camptothecin.

1182. The medical device of item 1005, further comprising an agent that inhibits infection, wherein the agent is a hydroxyurea.

1183. The medical device of item 1005, further comprising an agent that inhibits infection, wherein the agent is a platinum complex.

1184. The medical device of item 1005, further comprising an agent that inhibits infection, wherein the agent is cisplatin.

1185. The medical device of item 1005, further comprising an anti-thrombotic agent.

1186. The medical device of item 1005, further comprising a visualization agent.

1187. The medical device of item 1005, further comprising a visualization agent, wherein the visualization agent is a radiopaque material, wherein the radiopaque material comprises a metal, a halogenated compound, or a barium containing compound.

1188. The medical device of item 1005, further comprising a visualization agent, wherein the visualization agent is a radiopaque material, wherein the radiopaque material comprises barium, tantalum, or technetium.

1189. The medical device of item 1005, further comprising a visualization agent, wherein the visualization agent is a MRI responsive material.

1190. The medical device of item 1005, further comprising a visualization agent, wherein the visualization agent comprises a gadolinium chelate.

1191. The medical device of item 1005, further comprising a visualization agent, wherein the visualization agent comprises iron, magnesium, manganese, copper, or chromium.

1192. The medical device of item 1005, further comprising a visualization agent, wherein the visualization agent comprises an iron oxide compound.

1193. The medical device of item 1005, further comprising a visualization agent, wherein the visualization agent comprises a dye, pigment, or colorant.

1194. The medical device of item 1005, further comprising an echogenic material.

1195. The medical device of item 1005, further comprising an echogenic material, wherein the echogenic material is in the form of a coating.

1196. The medical device of item 1005 wherein the device is sterile.

1197. The medical device of item 1005 wherein the anti-scarring agent inhibits adhesion between the medical device and a host into which the medical device is implanted.

1198. The medical device of item 1005 wherein the medical device delivers the anti-scarring agent locally to tissue proximate to the medical device.

1199. The medical device of item 1005 wherein the anti-scarring agent is released into tissue in the vicinity of the medical device after deployment of the medical device.

1200. The medical device of item 1005 wherein the anti-scarring agent is released into tissue in the vicinity of the medical device after deployment of the medical device, wherein the tissue is connective tissue.

1201. The medical device of item 1005 wherein the anti-scarring agent is released into tissue in the vicinity of the medical device after deployment of the medical device, wherein the tissue is muscle tissue.

1202. The medical device of item 1005 wherein the anti-scarring agent is released into tissue in the vicinity of the medical device after deployment of the medical device, wherein the tissue is nerve tissue.

1203. The medical device of item 1005 wherein the anti-scarring agent is released into tissue in the vicinity of the medical device after deployment of the medical device, wherein the tissue is epithelium tissue.

1204. The medical device of item 1005 wherein the anti-scarring agent is released in effective concentrations from the medical device over a period ranging from the time of deployment of the medical device to about 1 year.

1205. The medical device of item 1005 wherein the anti-scarring agent is released in effective concentrations from the medical device over a period ranging from about 1 month to 6 months.

1206. The medical device of item 1005 wherein the anti-scarring agent is released in effective concentrations from the medical device over a period ranging from about 1-90 days.

1207. The medical device of item 1005 wherein the anti-scarring agent is released in effective concentrations from the medical device at a constant rate.

1208. The medical device of item 1005 wherein the anti-scarring agent is released in effective concentrations from the medical device at an increasing rate.

1209. The medical device of item 1005 wherein the anti-scarring agent is released in effective concentrations from the medical device at a decreasing rate.

1210. The medical device of item 1005 wherein the anti-scarring agent is released in effective concentrations from the composition comprising the anti-scarring agent by diffusion over a period ranging from the time of deployment of the medical device to about 90 days.

1211. The medical device of item 1005 wherein the anti-scarring agent is released in effective concentrations from the composition comprising the anti-scarring agent by erosion of the composition over a period ranging from the time of deployment of the medical device to about 90 days.

1212. The medical device of item 1005 wherein the device comprises about 0.01 μg to about 10 μg of the anti-scarring agent.

1213. The medical device of item 1005 wherein the device comprises about 10 μg to about 10 mg of the anti-scarring agent.

1214. The medical device of item 1005 wherein the device comprises about 10 mg to about 250 mg of the anti-scarring agent.

1215. The medical device of item 1005 wherein the device comprises about 250 mg to about 1000 mg of the anti-scarring agent.

1216. The medical device of item 1005 wherein the device comprises about 1000 mg to about 2500 mg of the anti-scarring agent.

1217. The medical device of item 1005 wherein a surface of the device comprises less than 0.01 μg of the anti-scarring agent per mm² of device surface to which the anti-scarring agent is applied.

1218. The medical device of item 1005 wherein a surface of the device comprises about 0.01 μg to about 1 μg of the anti-scarring agent per mm² of device surface to which the anti-scarring agent is applied.

1219. The medical device of item 1005 wherein a surface of the device comprises about 1 μg to about 10 μg of the anti-scarring agent per mm² of device surface to which the anti-scarring agent is applied.

1220. The medical device of item 1005 wherein a surface of the device comprises about 10 μg to about 250 μg of the anti-scarring agent per mm² of device surface to which the anti-scarring agent is applied.

1221. The medical device of item 1005 wherein a surface of the device comprises about 250 μg to about 1000 μg of the anti-scarring agent of anti-scarring agent per mm² of device surface to which the anti-scarring agent is applied.

1222. The medical device of item 1005 wherein a surface of the device comprises about 1000 μg to about 2500 μg of the anti-scarring agent per mm² of device surface to which the anti-scarring agent is applied.

1223. The medical device of item 1005 wherein the agent or the composition is affixed to the electrical device.

1224. The medical device of item 1005 wherein the agent or the composition is covalently attached to the electrical device.

1225. The medical device of item 1005 wherein the agent or the composition is non-covalently attached to the electrical device.

1226. The medical device of item 1005 further comprising a coating that absorbs the agent or the composition.

1227. The medical device of item 1005 wherein the electrical device is interweaved with a thread composed of, or coated with, the agent or the composition.

1228. The medical device of item 1005 wherein a portion of the electrical device is covered with a sleeve that contains the agent or the composition.

1229. The medical device of item 1005 wherein the electrical device is completely covered with a sleeve that contains the agent or the composition.

1230. The medical device of item 1005 wherein a portion of the electrical device is covered with a mesh that contains the agent or the composition.

1231. The medical device of item 1005 wherein the electrical device is completely covered with a mesh that contains the agent or the composition.

1232. The medical device of any one of items 1005-1231 wherein the vagal nerve stimulator is adapted for treating or preventing depression.

1233. The medical device of any one of items 1005-1231 wherein the vagal nerve stimulator is adapted for treating or preventing anxiety.

1234. The medical device of any one of items 1005-1231 wherein the vagal nerve stimulator is adapted for treating or preventing panic disorders.

1235. The medical device of any one of items 1005-1231 wherein the vagal nerve stimulator is adapted for treating or preventing obsessive-compulsive disorders.

1236. The medical device of any one of items 1005-1231 wherein the vagal nerve stimulator is adapted for treating or preventing post-traumatic disorders.

1237. The medical device of any one of items 1005-1231 wherein the vagal nerve stimulator is adapted for treating or preventing obesity.

1238. The medical device of any one of items 1005-1231 wherein the vagal nerve stimulator is adapted for treating or preventing migraine.

1239. The medical device of any one of items 1005-1231 wherein the vagal nerve stimulator is adapted for treating or preventing sleep disorders.

1240. The medical device of any one of items 1005-1231 wherein the vagal nerve stimulator is adapted for treating or preventing dementia.

1241. The medical device of any one of items 1005-1231 wherein the vagal nerve stimulator is adapted for treating or preventing Alzheimer's disease.

1242. The medical device of any one of items 1005-1231 wherein the vagal nerve stimulator is adapted for treating or preventing chronic or degenerative neurological disorders.

1243. A medical device, comprising a sacral nerve stimulator for treating a bladder control problem (i.e., an electrical device) and an anti-scarring agent or a composition comprising an anti-scarring agent, wherein the agent inhibits scarring between the medical device and the host into which the medical device is implanted.

1244. The medical device of item 1243 wherein the agent is an adensosine A2A receptor antagonist.

1245. The medical device of item 1243 wherein the agent is an AKT inhibitor.

1246. The medical device of item 1243 wherein the agent is an alpha 2 integrin antagonist, wherein the alpha 2 integrin antagonist is Pharmaprojects No. 5754 (Merck KgaA).

1247. The medical device of item 1243 wherein the agent is an alpha 4 integrin antagonist.

1248. The medical device of item 1243 wherein the agent is an alpha 7 nicotinic receptor agonist.

1249. The medical device of item 1243 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

1250. The medical device of item 1243 wherein the agent is an apoptosis antagonist.

1251. The medical device of item 1243 wherein the agent is an apoptosis activator.

1252. The medical device of item 1243 wherein the agent is a beta 1 integrin antagonist.

1253. The medical device of item 1243 wherein the agent is a beta tubulin inhibitor.

1254. The medical device of item 1243 wherein the agent is a blocker of enzyme production in Hepatitis C.

1255. The medical device of item 1243 wherein the agent is a Bruton's tyrosine kinase inhibitor.

1256. The medical device of item 1243 wherein the agent is a calcineurin inhibitor.

1257. The medical device of item 1243 wherein the agent is a caspase 3 inhibitor.

1258. The medical device of item 1243 wherein the agent is a CC chemokine receptor antagonist.

1259. The medical device of item 1243 wherein the agent is a cell cycle inhibitor selected from the group consisting of SNS-595 (Sunesis), synthadotin, KRX-0403, and an analogue or derivative thereof.

1260. The medical device of item 1243 wherein the agent is a cathepsin B inhibitor.

1261. The medical device of item 1243 wherein the agent is a cathepsin K inhibitor, wherein the cathepsin K inhibitor is 462795 (GlaxoSmithKline), INPL-022-D6 (Amura Therapeutics), or an analogue or derivative thereof.

1262. The medical device of item 1243 wherein the agent is a cathepsin L inhibitor.

1263. The medical device of item 1243 wherein the agent is a CD40 antagonist.

1264. The medical device of item 1243 wherein the agent is a chemokine receptor agonist.

1265. The medical device of item 1243 wherein the agent is a chymase inhibitor.

1266. The medical device of item 1243 wherein the agent is a collagenase antagonist.

1267. The medical device of item 1243 wherein the agent is a CXCR antagonist.

1268. The medical device of item 1243 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

1269. The medical device of item 1243 wherein the agent is a cyclooxygenase 1 inhibitor.

1270. The medical device of item 1243 wherein the agent is a DHFR inhibitor.

1271. The medical device of item 1243 wherein the agent is a dual integrin inhibitor.

1272. The medical device of item 1243 wherein the agent is an elastase inhibitor.

1273. The medical device of item 1243 wherein the agent is an elongation factor-1 alpha inhibitor.

1274. The medical device of item 1243 wherein the agent is an endothelial growth factor antagonist.

1275. The medical device of item 1243 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

1276. The medical device of item 1243 wherein the agent is an endotoxin antagonist.

1277. The medical device of item 1243 wherein the agent is an epothilone and tubulin binder.

1278. The medical device of item 1243 wherein the agent is an estrogen receptor antagonist.

1279. The medical device of item 1243 wherein the agent is an FGF inhibitor.

1280. The medical device of item 1243 wherein the agent is a farnexyl transferase inhibitor

1281. The medical device of item 1243 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

1282. The medical device of item 1243 wherein the agent is an FLT-3 kinase inhibitor

1283. The medical device of item 1243 wherein the agent is an FGF receptor kinase inhibitor.

1284. The medical device of item 1243 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

1285. The medical device of item 1243 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

1286. The medical device of item 1243 wherein the agent is a histone deacetylase inhibitor.

1287. The medical device of item 1243 wherein the agent is an HMGCoA reductase inhibitor selected from the group consisting of an atherosclerosis therapeutic from Lipid Sciences, ATI-16000 (ARYx

Therapeutics), KS-01-019 (Kos Pharmaceuticals), Pharmaprojects No. 2197 (Sanofi-Aventi), RP 61969 (Sanofi-Aventis), and an analogue or derivative thereof.

1288. The medical device of item 1243 wherein the agent is an ICAM inhibitor.

1289. The medical device of item 1243 wherein the agent is an IL, ICE and IRAK antagonist, wherein the antagonist is a CJ-14877, CP-424174 (Pfizer), NF-61 (Negma-Lerads), and an analogue or derivative thereof.

1290. The medical device of item 1243 wherein the agent is an IL-2 inhibitor.

1291. The medical device of item 1243 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

1292. The medical device of item 1243 wherein the agent is an IMPDH (inosine monophosphate).

1293. The medical device of item 1243 wherein the agent is an integrin antagonist.

1294. The medical device of item 1243 wherein the agent is an interleukin antagonist.

1295. The medical device of item 1243 wherein the agent is an inhibitor of type III receptor tyrosine kinase.

1296. The medical device of item 1243 wherein the agent is an irreversible inhibitor of enzyme methionine aminopeptidase type 2.

1297. The medical device of item 1243 wherein the agent is an isozyme selective delta protein kinase C inhibitor.

1298. The medical device of item 1243 wherein the agent a JAK3 enzyme inhibitor.

1299. The medical device of item 1243 wherein the agent is a JNK inhibitor.

1300. The medical device of item 1243 wherein the agent is a kinase inhibitor.

1301. The medical device of item 1243 wherein the agent is kinesin antagonist.

1302. The medical device of item 1243 wherein the agent is a kinesin antagonist.

1303. The medical device of item 1243 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 1119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

1304. The medical device of item 1243 wherein the agent is an MAP kinase inhibitor.

1305. The medical device of item 1243 wherein the agent is a matrix metalloproteinase inhibitor.

1306. The medical device of item 1243 wherein the agent is an MCP-CCR2 inhibitor.

1307. The medical device of item 1243 wherein the agent is an mTOR inhibitor.

1308. The medical device of item 1243 wherein the agent is an mTOR kinase inhibitor.

1309. The medical device of item 1243 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

1310. The medical device of item 1243 wherein the agent is an MIF inhibitor.

1311. The medical device of item 1243 wherein the agent is an MMP inhibitor.

1312. The medical device of item 1243 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

1313. The medical device of item 1243 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

1314. The medical device of item 1243 wherein the agent is a nitric oxide agonist.

1315. The medical device of item 1243 wherein the agent is an ornithine decarboxylase inhibitor.

1316. The medical device of item 1243 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

1317. The medical device of item 1243 wherein the agent is a palmitoyl-protein thioesterase inhibitor.

1318. The medical device of item 1243 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

1319. The medical device of item 1243 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

1320. The medical device of item 1243 wherein the agent is a phosphatase inhibitor.

1321. The medical device of item 1243 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

1322. The medical device of item 1243 wherein the agent is a PKC inhibitor.

1323. The medical device of item 1243 wherein the agent is a platelet activating factor antagonist.

1324. The medical device of item 1243 wherein the agent is a platelet-derived growth factor receptor kinase inhibitor.

1325. The medical device of item 1243 wherein the agent is a prolyl hydroxylase inhibitor.

1326. The medical device of item 1243 wherein the agent is a polymorphonuclear neutrophil inhibitor.

1327. The medical device of item 1243 wherein the agent is a protein kinase B inhibitor.

1328. The medical device of item 1243 wherein the agent is a protein kinase C stimulant.

1329. The medical device of item 1243 wherein the agent is a purine nucleoside analogue.

1330. The medical device of item 1243 wherein the agent is a purinoreceptor P2X antagonist.

1331. The medical device of item 1243 wherein the agent is a Raf kinase inhibitor.

1332. The medical device of item 1243 wherein the agent is a reversible inhibitor of ErbB1 and ErbB2.

1333. The medical device of item 1243 wherein the agent is a ribonucleoside triphosphate reductase inhibitor.

1334. The medical device of item 1243 wherein the agent is an SDF-1 antagonist.

1335. The medical device of item 1243 wherein the agent is a sheddase inhibitor.

1336. The medical device of item 1243 wherein the agent is an SRC inhibitor.

1337. The medical device of item 1243 wherein the agent is a stromelysin inhibitor.

1338. The medical device of item 1243 wherein the agent is an Syk kinase inhibitor.

1339. The medical device of item 1243 wherein the agent is a telomerase inhibitor.

1340. The medical device of item 1243 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), ma nnose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

1341. The medical device of item 1243 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-1 38 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

1342. The medical device of item 1243 wherein the agent is a Toll receptor inhibitor.

1343. The medical device of item 1243 wherein the agent is a tubulin antagonist.

1344. The medical device of item 1243 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

1345. The medical device of item 1243 wherein the agent is a VEGF inhibitor.

1346. The medical device of item 1243 wherein the agent is a vitamin D receptor agonist.

1347. The medical device of item 1243 wherein the agent is ZD-6474 (an angiogenesis inhibitor).

1348. The medical device of item 1243 wherein the agent is AP-23573 (an mTOR inhibitor).

1349. The medical device of item 1243 wherein the agent is synthadotin (a tubulin antagonist).

1350. The medical device of item 1243 wherein the agent is S-0885 (a collagenase inhibitor).

1351. The medical device of item 1243 wherein the agent is aplidine (an elongation factor-1 alpha inhibitor).

1352. The medical device of item 1243 wherein the agent is ixabepilone (an epithilone).

1353. The medical device of item 1243 wherein the agent is IDN-5390 (an angiogenesis inhibitor).

1354. The medical device of item 1243 wherein the agent is SB-2723005 (an angiogenesis inhibitor).

1355. The medical device of item 1243 wherein the agent is ABT-518 (an angiogenesis inhibitor).

1356. The medical device of item 1243 wherein the agent is combretastatin (an angiogenesis inhibitor).

1357. The medical device of item 1243 wherein the agent is anecortave acetate (an angiogenesis inhibitor).

1358. The medical device of item 1243 wherein the agent is SB-715992 (a kinesin antagonist).

1359. The medical device of item 1243 wherein the agent is temsirolimus (an mTOR inhibitor).

1360. The medical device of item 1243 wherein the agent is adalimumab (a TNFα antagonist).

1361. The medical device of item 1243, further comprising a coating, wherein the coating comprises the anti-scarring agent and a polymer.

1362. The medical device of item 1243, further comprising a coating, wherein the coating comprises the anti-scarring agent.

1363. The medical device of item 1243, further comprising a coating, wherein the coating is disposed on a surface of the electrical device.

1364. The medical device of item 1243, further comprising a coating, wherein the coating directly contacts the electrical device.

1365. The medical device of item 1243, further comprising a coating, wherein the coating indirectly contacts the electrical device.

1366. The medical device of item 1243, further comprising a coating, wherein the coating partially covers the electrical device.

1367. The medical device of item 1243, further comprising a coating, wherein the coating completely covers the electrical device.

1368. The medical device of item 1243, further comprising a coating, wherein the coating is a uniform coating.

1369. The medical device of item 1243, further comprising a coating, wherein the coating is a non-uniform coating.

1370. The medical device of item 1243, further comprising a coating, wherein the coating is a discontinuous coating.

1371. The medical device of item 1243, further comprising a coating, wherein the coating is a patterned coating.

1372. The medical device of item 1243, further comprising a coating, wherein the coating has a thickness of 100 μm or less.

1373. The medical device of item 1243, further comprising a coating, wherein the coating has a thickness of 10 μm or less.

1374. The medical device of item 1243, further comprising a coating, wherein the coating adheres to the surface of the electrical device upon deployment of the medical device.

1375. The medical device of item 1243, further comprising a coating, wherein the coating is stable at room temperature for a period of 1 year.

1376. The medical device of item 1243, further comprising a coating, wherein the anti-scarring agent is present in the coating in an amount ranging between about 0.0001% to about 1% by weight.

1377. The medical device of item 1243, further comprising a coating, wherein the anti-scarring agent is present in the coating in an amount ranging between about 1% to about 10% by weight.

1378. The medical device of item 1243, further comprising a coating, wherein the anti-scarring agent is present in the coating in an amount ranging between about 10% to about 25% by weight.

1379. The medical device of item 1243, further comprising a coating, wherein the anti-scarring agent is present in the coating in an amount ranging between about 25% to about 70% by weight.

1380. The medical device of item 1243, further comprising a coating, wherein the coating further comprises a polymer.

1381. The medical device of item 1243, further comprising a first coating having a first composition and the second coating having a second composition.

1382. The medical device of item 1243, further comprising a first coating having a first composition and the second coating having a second composition, wherein the first composition and the second composition are different.

1383. The medical device of item 1243, further comprising a polymer.

1384. The medical device of item 1243, further comprising a polymeric carrier.

1385. The medical device of item 1243, further comprising a polymeric carrier, wherein the polymeric carrier comprises a copolymer.

1386. The medical device of item 1243, further comprising a polymeric carrier, wherein the polymeric carrier comprises a block copolymer.

1387. The medical device of item 1243, further comprising a polymeric carrier, wherein the polymeric carrier comprises a random copolymer.

1388. The medical device of item 1243, further comprising a polymeric carrier, wherein the polymeric carrier comprises a biodegradable polymer.

1389. The medical device of item 1243, further comprising a polymeric carrier, wherein the polymeric carrier comprises a non-biodegradable polymer.

1390. The medical device of item 1243, further comprising a polymeric carrier, wherein the polymeric carrier comprises a hydrophilic polymer.

1391. The medical device of item 1243, further comprising a polymeric carrier, wherein the polymeric carrier comprises a hydrophobic polymer.

1392. The medical device of item 1243, further comprising a polymeric carrier, wherein the polymeric carrier comprises a polymer having hydrophilic domains.

1393. The medical device of item 1243, further comprising a polymeric carrier, wherein the polymeric carrier comprises a polymer having hydrophobic domains.

1394. The medical device of item 1243, further comprising a polymeric carrier, wherein the polymeric carrier comprises a non-conductive polymer.

1395. The medical device of item 1243, further comprising a polymeric carrier, wherein the polymeric carrier comprises an elastomer.

1396. The medical device of item 1243, further comprising a polymeric carrier, wherein the polymeric carrier comprises a hydrogel.

1397. The medical device of item 1243, further comprising a polymeric carrier, wherein the polymeric carrier comprises a silicone polymer.

1398. The medical device of item 1243, further comprising a polymeric carrier, wherein the polymeric carrier comprises a hydrocarbon polymer.

1399. The medical device of item 1243, further comprising a polymeric carrier, wherein the polymeric carrier comprises a styrene-derived polymer.

1400. The medical device of item 1243, further comprising a polymeric carrier, wherein the polymeric carrier comprises a butadiene polymer.

1401. The medical device of item 1243, further comprising a polymeric carrier, wherein the polymeric carrier comprises a macromer.

1402. The medical device of item 1243, further comprising a polymeric carrier, wherein the polymeric carrier comprises a poly(ethylene glycol) polymer.

1403. The medical device of item 1243, further comprising a polymeric carrier, wherein the polymeric carrier comprises an amorphous polymer.

1404. The medical device of item 1243, further comprising a lubricious coating.

1405. The medical device of item 1243 wherein the anti-scarring agent is located within pores or holes of the electrical device.

1406. The medical device of item 1243 wherein the anti-scarring agent is located within a channel, lumen, or divet of the electrical device.

1407. The medical device of item 1243, further comprising a second pharmaceutically active agent.

1408. The medical device of item 1243, further comprising an anti-inflammatory agent.

1409. The medical device of item 1243, further comprising an agent that inhibits infection.

1410. The medical device of item 1243, further comprising an agent that inhibits infection, wherein the agent is an anthracycline.

1411. The medical device of item 1243, further comprising an agent that inhibits infection, wherein the agent is doxorubicin.

1412. The medical device of item 1243, further comprising an agent that inhibits infection, wherein the agent is mitoxantrone.

1413. The medical device of item 1243, further comprising an agent that inhibits infection, wherein the agent is a fluoropyrimidine.

1414. The medical device of item 1243, further comprising an agent that inhibits infection, wherein the agent is 5-fluorouracil (5-FU).

1415. The medical device of item 1243, further comprising an agent that inhibits infection, wherein the agent is a folic acid antagonist.

1416. The medical device of item 1243, further comprising an agent that inhibits infection, wherein the agent is methotrexate.

1417. The medical device of item 1243, further comprising an agent that inhibits infection, wherein the agent is a podophylotoxin.

1418. The medical device of item 1243, further comprising an agent that inhibits infection, wherein the agent is etoposide.

1419. The medical device of item 1243, further comprising an agent that inhibits infection, wherein the agent is a camptothecin.

1420. The medical device of item 1243, further comprising an agent that inhibits infection, wherein the agent is a hydroxyurea.

1421. The medical device of item 1243, further comprising an agent that inhibits infection, wherein the agent is a platinum complex.

1422. The medical device of item 1243, further comprising an agent that inhibits infection, wherein the agent is cisplatin.

1423. The medical device of item 1243, further comprising an anti-thrombotic agent.

1424. The medical device of item 1243, further comprising a visualization agent.

1425. The medical device of item 1243, further comprising a visualization agent, wherein the visualization agent is a radiopaque material, wherein the radiopaque material comprises a metal, a halogenated compound, or a barium containing compound.

1426. The medical device of item 1243, further comprising a visualization agent, wherein the visualization agent is a radiopaque material, wherein the radiopaque material comprises barium, tantalum, or technetium.

1427. The medical device of item 1243, further comprising a visualization agent, wherein the visualization agent is a MRI responsive material.

1428. The medical device of item 1243, further comprising a visualization agent, wherein the visualization agent comprises a gadolinium chelate.

1429. The medical device of item 1243, further comprising a visualization agent, wherein the visualization agent comprises iron, magnesium, manganese, copper, or chromium

1430. The medical device of item 1243, further comprising a visualization agent, wherein the visualization agent comprises an iron oxide compound.

1431. The medical device of item 1243, further comprising a visualization agent, wherein the visualization agent comprises a dye, pigment, or colorant.

1432. The medical device of item 1243, further comprising an echogenic material.

1433. The medical device of item 1243, further comprising an echogenic material, wherein the echogenic material is in the form of a coating.

1434. The medical device of item 1243 wherein the device is sterile.

1435. The medical device of item 1243 wherein the anti-scarring agent inhibits adhesion between the medical device and a host into which the medical device is implanted.

1436. The medical device of item 1243 wherein the medical device delivers the anti-scarring agent locally to tissue proximate to the medical device.

1437. The medical device of item 1243 wherein the anti-scarring agent is released into tissue in the vicinity of the medical device after deployment of the medical device.

1438. The medical device of item 1243 wherein the anti-scarring agent is released into tissue in the vicinity of the medical device after deployment of the medical device, wherein the tissue is connective tissue.

1439. The medical device of item 1243 wherein the anti-scarring agent is released into tissue in the vicinity of the medical device after deployment of the medical device, wherein the tissue is muscle tissue.

1440. The medical device of item 1243 wherein the anti-scarring agent is released into tissue in the vicinity of the medical device after deployment of the medical device, wherein the tissue is nerve tissue.

1441. The medical device of item 1243 wherein the anti-scarring agent is released into tissue in the vicinity of the medical device after deployment of the medical device, wherein the tissue is epithelium tissue.

1442. The medical device of item 1243 wherein the anti-scarring agent is released in effective concentrations from the medical device over a period ranging from the time of deployment of the medical device to about 1 year.

1443. The medical device of item 1243 wherein the anti-scarring agent is released in effective concentrations from the medical device over a period ranging from about 1 month to 6 months.

1444. The medical device of item 1243 wherein the anti-scarring agent is released in effective concentrations from the medical device over a period ranging from about 1-90 days.

1445. The medical device of item 1243 wherein the anti-scarring agent is released in effective concentrations from the medical device at a constant rate.

1446. The medical device of item 1243 wherein the anti-scarring agent is released in effective concentrations from the medical device at an increasing rate.

1447. The medical device of item 1243 wherein the anti-scarring agent is released in effective concentrations from the medical device at a decreasing rate.

1448. The medical device of item 1243 wherein the anti-scarring agent is released in effective concentrations from the composition comprising the anti-scarring agent by diffusion over a period ranging from the time of deployment of the medical device to about 90 days.

1449. The medical device of item 1243 wherein the anti-scarring agent is released in effective concentrations from the composition comprising the anti-scarring agent by erosion of the composition over a period ranging from the time of deployment of the medical device to about 90 days.

1450. The medical device of item 1243 wherein the device comprises about 0.01 μg to about 10 μg of the anti-scarring agent.

1451. The medical device of item 1243 wherein the device comprises about 10 μg to about 10 mg of the anti-scarring agent.

1452. The medical device of item 1243 wherein the device comprises about 10 mg to about 250 mg of the anti-scarring agent.

1453. The medical device of item 1243 wherein the device comprises about 250 mg to about 1000 mg of the anti-scarring agent.

1454. The medical device of item 1243 wherein the device comprises about 1000 mg to about 2500 mg of the anti-scarring agent.

1455. The medical device of item 1243 wherein a surface of the device comprises less than 0.01 μg of the anti-scarring agent per mm² of device surface to which the anti-scarring agent is applied.

1456. The medical device of item 1243 wherein a surface of the device comprises about 0.01 μg to about 1 μg of the anti-scarring agent per mm² of device surface to which the anti-scarring agent is applied.

1457. The medical device of item 1243 wherein a surface of the device comprises about 1 μg to about 10 μg of the anti-scarring agent per mm² of device surface to which the anti-scarring agent is applied.

1458. The medical device of item 1243 wherein a surface of the device comprises about 10 μg to about 250 μg of the anti-scarring agent per mm² of device surface to which the anti-scarring agent is applied.

1459. The medical device of item 1243 wherein a surface of the device comprises about 250 μg to about 1000 μg of the anti-scarring agent of anti-scarring agent per mm² of device surface to which the anti-scarring agent is applied.

1460. The medical device of item 1243 wherein a surface of the device comprises about 1000 μg to about 2500 μg of the anti-scarring agent per mm² of device surface to which the anti-scarring agent is applied.

1461. The medical device of item 1243 wherein the agent or the composition is affixed to the electrical device.

1462. The medical device of item 1243 wherein the agent or the composition is covalently attached to the electrical device.

1463. The medical device of item 1243 wherein the agent or the composition is non-covalently attached to the electrical device.

1464. The medical device of item 1243 further comprising a coating that absorbs the agent or the composition.

1465. The medical device of item 1243 wherein the electrical device is interweaved with a thread composed of, or coated with, the agent or the composition.

1466. The medical device of item 1243 wherein a portion of the electrical device is covered with a sleeve that contains the agent or the composition.

1467. The medical device of item 1243 wherein the electrical device is completely covered with a sleeve that contains the agent or the composition.

1468. The medical device of item 1243 wherein a portion of the electrical device is covered with a mesh that contains the agent or the composition.

1469. The medical device of item 1243 wherein the electrical device is completely covered with a mesh that contains the agent or the composition.

1470. The medical device of any one of items 1243-1469 wherein the sacral nerve stimulator is adapted for treating or preventing urge incontinence.

1471. The medical device of any one of items 1243-1469 wherein the sacral nerve stimulator is adapted for treating or preventing nonobstructive urinary retention.

1472. The medical device of any one of items 1243-1469 wherein the sacral nerve stimulator is adapted for treating or preventing urgency frequency.

1473. The medical device of any one of items 1243-1469 wherein the sacral nerve stimulator is an intramuscular electrical stimulator.

1474. The medical device of any one of items 1243-1469 wherein the sacral nerve stimulator is a leadless, tubular-shaped microstimulator.

1475. A medical device, comprising a gastric nerve stimulator for treating a gastrointestinal disorder (i.e., an electrical device) and an anti-scarring agent or a composition comprising an anti-scarring agent, wherein the agent inhibits scarring between the medical device and the host into which the medical device is implanted.

1476. The medical device of item 1475 wherein the agent is an adensosine A2A receptor antagonist.

1477. The medical device of item 1475 wherein the agent is an AKT inhibitor.

1478. The medical device of item 1475 wherein the agent is an alpha 2 integrin antagonist, wherein the alpha 2 integrin antagonist is Pharmaprojects No. 5754 (Merck KgaA).

1479. The medical device of item 1475 wherein the agent is an alpha 4 integrin antagonist.

1480. The medical device of item 1475 wherein the agent is an alpha 7 nicotinic receptor agonist.

1481. The medical device of item 1475 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

1482. The medical device of item 1475 wherein the agent is an apoptosis antagonist.

1483. The medical device of item 1475 wherein the agent is an apoptosis activator.

1484. The medical device of item 1475 wherein the agent is a beta 1 integrin antagonist.

1485. The medical device of item 1475 wherein the agent is a beta tubulin inhibitor.

1486. The medical device of item 1475 wherein the agent is a blocker of enzyme production in Hepatitis C.

1487. The medical device of item 1475 wherein the agent is a Bruton's tyrosine kinase inhibitor.

1488. The medical device of item 1475 wherein the agent is a calcineurin inhibitor.

1489. The medical device of item 1475 wherein the agent is a caspase 3 inhibitor.

1490. The medical device of item 1475 wherein the agent is a CC chemokine receptor antagonist.

1491. The medical device of item 1475 wherein the agent is a cell cycle inhibitor selected from the group consisting of SNS-595 (Sunesis), synthadotin, KRX-0403, and an analogue or derivative thereof.

1492. The medical device of item 1475 wherein the agent is a cathepsin B inhibitor.

1493. The medical device of item 1475 wherein the agent is a cathepsin K inhibitor, wherein the cathepsin K inhibitor is 462795 (GlaxoSmithKline), INPL-022-D6 (Amura Therapeutics), or an analogue or derivative thereof.

1494. The medical device of item 1475 wherein the agent is a cathepsin L inhibitor.

1495. The medical device of item 1475 wherein the agent is a CD40 antagonist.

1496. The medical device of item 1475 wherein the agent is a chemokine receptor agonist.

1497. The medical device of item 1475 wherein the agent is a chymase inhibitor.

1498. The medical device of item 1475 wherein the agent is a collagenase antagonist.

1499. The medical device of item 1475 wherein the agent is a CXCR antagonist.

1500. The medical device of item 1475 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

1501. The medical device of item 1475 wherein the agent is a cyclooxygenase 1 inhibitor.

1502. The medical device of item 1475 wherein the agent is a DHFR inhibitor.

1503. The medical device of item 1475 wherein the agent is a dual integrin inhibitor.

1504. The medical device of item 1475 wherein the agent is an elastase inhibitor.

1505. The medical device of item 1475 wherein the agent is an elongation factor-1 alpha inhibitor.

1506. The medical device of item 1475 wherein the agent is an endothelial growth factor antagonist.

1507. The medical device of item 1475 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof

1508. The medical device of item 1475 wherein the agent is an endotoxin antagonist.

1509. The medical device of item 1475 wherein the agent is an epothilone and tubulin binder.

1510. The medical device of item 1475 wherein the agent is an estrogen receptor antagonist.

1511. The medical device of item 1475 wherein the agent is an FGF inhibitor.

1512. The medical device of item 1475 wherein the agent is a farnexyl transferase inhibitor.

1513. The medical device of item 1475 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

1514. The medical device of item 1475 wherein the agent is an FLT-3 kinase inhibitor.

1515. The medical device of item 1475 wherein the agent is an FGF receptor kinase inhibitor.

1516. The medical device of item 1475 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

1517. The medical device of item 1475 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

1518. The medical device of item 1475 wherein the agent is a histone deacetylase inhibitor.

1519. The medical device of item 1475 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

1520. The medical device of item 1475 wherein the agent is an ICAM inhibitor.

1521. The medical device of item 1475 wherein the agent is an IL, ICE and IRAK antagonist, wherein the antagonist is a CJ-14877, CP-424174 (Pfizer), NF-61 (Negma-Lerads), and an analogue or derivative thereof.

1522. The medical device of item 1475 wherein the agent is an IL-2 inhibitor.

1523. The medical device of item 1475 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

1524. The medical device of item 1475 wherein the agent is an IMPDH (inosine monophosphate).

1525. The medical device of item 1475 wherein the agent is an integrin antagonist.

1526. The medical device of item 1475 wherein the agent is an interleukin antagonist.

1527. The medical device of item 1475 wherein the agent is an inhibitor of type III receptor tyrosine kinase.

1528. The medical device of item 1475 wherein the agent is an irreversible inhibitor of enzyme methionine aminopeptidase type 2.

1529. The medical device of item 1475 wherein the agent is an isozyme selective delta protein kinase C inhibitor.

1530. The medical device of item 1475 wherein the agent a JAK3 enzyme inhibitor.

1531. The medical device of item 1475 wherein the agent is a JNK inhibitor.

1532. The medical device of item 1475 wherein the agent is a kinase inhibitor.

1533. The medical device of item 1475 wherein the agent is kinesin antagonist.

1534. The medical device of item 1475 wherein the agent is a kinesin antagonist.

1535. The medical device of item 1475 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

1536. The medical device of item 1475 wherein the agent is an MAP kinase inhibitor.

1537. The medical device of item 1475 wherein the agent is a matrix metalloproteinase inhibitor.

1538. The medical device of item 1475 wherein the agent is an MCP-CCR2 inhibitor.

1539. The medical device of item 1475 wherein the agent is an mTOR inhibitor.

1540. The medical device of item 1475 wherein the agent is an mTOR kinase inhibitor.

1541. The medical device of item 1475 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

1542. The medical device of item 1475 wherein the agent is an MIF inhibitor.

1543. The medical device of item 1475 wherein the agent is an MMP inhibitor.

1544. The medical device of item 1475 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

1545. The medical device of item 1475 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

1546. The medical device of item 1475 wherein the agent is a nitric oxide agonist.

1547. The medical device of item 1475 wherein the agent is an ornithine decarboxylase inhibitor.

1548. The medical device of item 1475 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

1549. The medical device of item 1475 wherein the agent is a palmitoyl-protein thioesterase inhibitor.

1550. The medical device of item 1475 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

1551. The medical device of item 1475 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

1552. The medical device of item 1475 wherein the agent is a phosphatase inhibitor.

1553. The medical device of item 1475 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

1554. The medical device of item 1475 wherein the agent is a PKC inhibitor.

1555. The medical device of item 1475 wherein the agent is a platelet activating factor antagonist.

1556. The medical device of item 1475 wherein the agent is a platelet-derived growth factor receptor kinase inhibitor.

1557. The medical device of item 1475 wherein the agent is a prolyl hydroxylase inhibitor.

1558. The medical device of item 1475 wherein the agent is a polymorphonuclear neutrophil inhibitor.

1559. The medical device of item 1475 wherein the agent is a protein kinase B inhibitor.

1560. The medical device of item 1475 wherein the agent is a protein kinase C stimulant.

1561. The medical device of item 1475 wherein the agent is a purine nucleoside analogue.

1562. The medical device of item 1475 wherein the agent is a purinoreceptor P2X antagonist.

1563. The medical device of item 1475 wherein the agent is a Raf kinase inhibitor.

1564. The medical device of item 1475 wherein the agent is a reversible inhibitor of ErbB1 and ErbB2.

1565. The medical device of item 1475 wherein the agent is a ribonucleoside triphosphate reductase inhibitor.

1566. The medical device of item 1475 wherein the agent is an SDF-1 antagonist.

1567. The medical device of item 1475 wherein the agent is a sheddase inhibitor.

1568. The medical device of item 1475 wherein the agent is an SRC inhibitor.

1569. The medical device of item 1475 wherein the agent is a stromelysin inhibitor.

1570. The medical device of item 1475 wherein the agent is an Syk kinase inhibitor.

1571. The medical device of item 1475 wherein the agent is a telomerase inhibitor.

1572. The medical device of item 1475 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

1573. The medical device of item 1475 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from lpsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

1574. The medical device of item 1475 wherein the agent is a Toll receptor inhibitor.

1575. The medical device of item 1475 wherein the agent is a tubulin antagonist.

1576. The medical device of item 1475 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

1577. The medical device of item 1475 wherein the agent is a VEGF inhibitor.

1578. The medical device of item 1475 wherein the agent is a vitamin D receptor agonist.

1579. The medical device of item 1475 wherein the agent is ZD-6474 (an angiogenesis inhibitor).

1580. The medical device of item 1475 wherein the agent is AP-23573 (an mTOR inhibitor).

1581. The medical device of item 1475 wherein the agent is synthadotin (a tubulin antagonist).

1582. The medical device of item 1475 wherein the agent is S-0885 (a collagenase inhibitor).

1583. The medical device of item 1475 wherein the agent is aplidine (an elongation factor-1 alpha inhibitor).

1584. The medical device of item 1475 wherein the agent is ixabepilone (an epithilone).

1585. The medical device of item 1475 wherein the agent is IDN-5390 (an angiogenesis inhibitor).

1586. The medical device of item 1475 wherein the agent is SB-2723005 (an angiogenesis inhibitor).

1587. The medical device of item 1475 wherein the agent is ABT-518 (an angiogenesis inhibitor).

1588. The medical device of item 1475 wherein the agent is combretastatin (an angiogenesis inhibitor).

1589. The medical device of item 1475 wherein the agent is anecortave acetate (an angiogenesis inhibitor).

1590. The medical device of item 1475 wherein the agent is SB-715992 (a kinesin antagonist).

1591. The medical device of item 1475 wherein the agent is temsirolimus (an mTOR inhibitor).

1592. The medical device of item 1475 wherein the agent is adalimumab (a TNFα antagonist).

1593. The medical device of item 1475, further comprising a coating, wherein the coating comprises the anti-scarring agent and a polymer.

1594. The medical device of item 1475, further comprising a coating, wherein the coating comprises the anti-scarring agent.

1595. The medical device of item 1475, further comprising a coating, wherein the coating is disposed on a surface of the electrical device.

1596. The medical device of item 1475, further comprising a coating, wherein the coating directly contacts the electrical device.

1597. The medical device of item 1475, further comprising a coating, wherein the coating indirectly contacts the electrical device.

1598. The medical device of item 1475, further comprising a coating, wherein the coating partially covers the electrical device.

1599. The medical device of item 1475, further comprising a coating, wherein the coating completely covers the electrical device.

1600. The medical device of item 1475, further comprising a coating, wherein the coating is a uniform coating.

1601. The medical device of item 1475, further comprising a coating, wherein the coating is a non-uniform coating.

1602. The medical device of item 1475, further comprising a coating, wherein the coating is a discontinuous coating.

1603. The medical device of item 1475, further comprising a coating, wherein the coating is a patterned coating.

1604. The medical device of item 1475, further comprising a coating, wherein the coating has a thickness of 100 μm or less.

1605. The medical device of item 1475, further comprising a coating, wherein the coating has a thickness of 10 μm or less.

1606. The medical device of item 1475, further comprising a coating, wherein the coating adheres to the surface of the electrical device upon deployment of the medical device.

1607. The medical device of item 1475, further comprising a coating, wherein the coating is stable at room temperature for a period of 1 year.

1608. The medical device of item 1475, further comprising a coating, wherein the anti-scarring agent is present in the coating in an amount ranging between about 0.0001% to about 1% by weight.

1609. The medical device of item 1475, further comprising a coating, wherein the anti-scarring agent is present in the coating in an amount ranging between about 1% to about 10% by weight.

1610. The medical device of item 1475, further comprising a coating, wherein the anti-scarring agent is present in the coating in an amount ranging between about 10% to about 25% by weight.

1611. The medical device of item 1475, further comprising a coating, wherein the anti-scarring agent is present in the coating in an amount ranging between about 25% to about 70% by weight.

1612. The medical device of item 1475, further comprising a coating, wherein the coating further comprises a polymer.

1613. The medical device of item 1475, further comprising a first coating having a first composition and the second coating having a second composition.

1614. The medical device of item 1475, further comprising a first coating having a first composition and the second coating having a second composition, wherein the first composition and the second composition are different.

1615. The medical device of item 1475, further comprising a polymer.

1616. The medical device of item 1475, further comprising a polymeric carrier.

1617. The medical device of item 1475, further comprising a polymeric carrier, wherein the polymeric carrier comprises a copolymer.

1618. The medical device of item 1475, further comprising a polymeric carrier, wherein the polymeric carrier comprises a block copolymer.

1619. The medical device of item 1475, further comprising a polymeric carrier, wherein the polymeric carrier comprises a random copolymer.

1620. The medical device of item 1475, further comprising a polymeric carrier, wherein the polymeric carrier comprises a biodegradable polymer.

1621. The medical device of item 1475, further comprising a polymeric carrier, wherein the polymeric carrier comprises a non-biodegradable polymer.

1622. The medical device of item 1475, further comprising a polymeric carrier, wherein the polymeric carrier comprises a hydrophilic polymer.

1623. The medical device of item 1475, further comprising a polymeric carrier, wherein the polymeric carrier comprises a hydrophobic polymer.

1624. The medical device of item 1475, further comprising a polymeric carrier, wherein the polymeric carrier comprises a polymer having hydrophilic domains.

1625. The medical device of item 1475, further comprising a polymeric carrier, wherein the polymeric carrier comprises a polymer having hydrophobic domains.

1626. The medical device of item 1475, further comprising a polymeric carrier, wherein the polymeric carrier comprises a non-conductive polymer.

1627. The medical device of item 1475, further comprising a polymeric carrier, wherein the polymeric carrier comprises an elastomer.

1628. The medical device of item 1475, further comprising a polymeric carrier, wherein the polymeric carrier comprises a hydrogel.

1629. The medical device of item 1475, further comprising a polymeric carrier, wherein the polymeric carrier comprises a silicone polymer.

1630. The medical device of item 1475, further comprising a polymeric carrier, wherein the polymeric carrier comprises a hydrocarbon polymer.

1631. The medical device of item 1475, further comprising a polymeric carrier, wherein the polymeric carrier comprises a styrene-derived polymer.

1632. The medical device of item 1475, further comprising a polymeric carrier, wherein the polymeric carrier comprises a butadiene polymer.

1633. The medical device of item 1475, further comprising a polymeric carrier, wherein the polymeric carrier comprises a macromer.

1634. The medical device of item 1475, further comprising a polymeric carrier, wherein the polymeric carrier comprises a poly(ethylene glycol) polymer.

1635. The medical device of item 1475, further comprising a polymeric carrier, wherein the polymeric carrier comprises an amorphous polymer.

1636. The medical device of item 1475, further comprising a lubricious coating.

1637. The medical device of item 1475 wherein the anti-scarring agent is located within pores or holes of the electrical device.

1638. The medical device of item 1475 wherein the anti-scarring agent is located within a channel, lumen, or divet of the electrical device.

1639. The medical device of item 1475, further comprising a second pharmaceutically active agent.

1640. The medical device of item 1475, further comprising an anti-inflammatory agent.

1641. The medical device of item 1475, further comprising an agent that inhibits infection.

1642. The medical device of item 1475, further comprising an agent that inhibits infection, wherein the agent is an anthracycline.

1643. The medical device of item 1475, further comprising an agent that inhibits infection, wherein the agent is doxorubicin.

1644. The medical device of item 1475, further comprising an agent that inhibits infection, wherein the agent is mitoxantrone.

1645. The medical device of item 1475, further comprising an agent that inhibits infection, wherein the agent is a fluoropyrimidine.

1646. The medical device of item 1475, further comprising an agent that inhibits infection, wherein the agent is 5-fluorouracil (5-FU).

1647. The medical device of item 1475, further comprising an agent that inhibits infection, wherein the agent is a folic acid antagonist.

1648. The medical device of item 1475, further comprising an agent that inhibits infection, wherein the agent is methotrexate.

1649. The medical device of item 1475, further comprising an agent that inhibits infection, wherein the agent is a podophylotoxin.

1650. The medical device of item 1475, further comprising an agent that inhibits infection, wherein the agent is etoposide.

1651. The medical device of item 1475, further comprising an agent that inhibits infection, wherein the agent is a camptothecin.

1652. The medical device of item 1475, further comprising an agent that inhibits infection, wherein the agent is a hydroxyurea.

1653. The medical device of item 1475, further comprising an agent that inhibits infection, wherein the agent is a platinum complex.

1654. The medical device of item 1475, further comprising an agent that inhibits infection, wherein the agent is cisplatin.

1655. The medical device of item 1475, further comprising an anti-thrombotic agent.

1656. The medical device of item 1475, further comprising a visualization agent.

1657. The medical device of item 1475, further comprising a visualization agent, wherein the visualization agent is a radiopaque material, wherein the radiopaque material comprises a metal, a halogenated compound, or a barium containing compound.

1658. The medical device of item 1475, further comprising a visualization agent, wherein the visualization agent is a radiopaque material, wherein the radiopaque material comprises barium, tantalum, or technetium.

1659. The medical device of item 1475, further comprising a visualization agent, wherein the visualization agent is a MRI responsive material.

1660. The medical device of item 1475, further comprising a visualization agent, wherein the visualization agent comprises a gadolinium chelate.

1661. The medical device of item 1475, further comprising a visualization agent, wherein the visualization agent comprises iron, magnesium, manganese, copper, or chromium.

1662. The medical device of item 1475, further comprising a visualization agent, wherein the visualization agent comprises an iron oxide compound.

1663. The medical device of item 1475, further comprising a visualization agent, wherein the visualization agent comprises a dye, pigment, or colorant.

1664. The medical device of item 1475, further comprising an echogenic material.

1665. The medical device of item 1475, further comprising an echogenic material, wherein the echogenic material is in the form of a coating.

1666. The medical device of item 1475 wherein the device is sterile.

1667. The medical device of item 1475 wherein the anti-scarring agent inhibits adhesion between the medical device and a host into which the medical device is implanted.

1668. The medical device of item 1475 wherein the medical device delivers the anti-scarring agent locally to tissue proximate to the medical device.

1669. The medical device of item 1475 wherein the anti-scarring agent is released into tissue in the vicinity of the medical device after deployment of the medical device.

1670. The medical device of item 1475 wherein the anti-scarring agent is released into tissue in the vicinity of the medical device after deployment of the medical device, wherein the tissue is connective tissue.

1671. The medical device of item 1475 wherein the anti-scarring agent is released into tissue in the vicinity of the medical device after deployment of the medical device, wherein the tissue is muscle tissue.

1672. The medical device of item 1475 wherein the anti-scarring agent is released into tissue in the vicinity of the medical device after deployment of the medical device, wherein the tissue is nerve tissue.

1673. The medical device of item 1475 wherein the anti-scarring agent is released into tissue in the vicinity of the medical device after deployment of the medical device, wherein the tissue is epithelium tissue.

1674. The medical device of item 1475 wherein the anti-scarring agent is released in effective concentrations from the medical device over a period ranging from the time of deployment of the medical device to about 1 year.

1675. The medical device of item 1475 wherein the anti-scarring agent is released in effective concentrations from the medical device over a period ranging from about 1 month to 6 months.

1676. The medical device of item 1475 wherein the anti-scarring agent is released in effective concentrations from the medical device over a period ranging from about 1-90 days.

1677. The medical device of item 1475 wherein the anti-scarring agent is released in effective concentrations from the medical device at a constant rate.

1678. The medical device of item 1475 wherein the anti-scarring agent is released in effective concentrations from the medical device at an increasing rate.

1679. The medical device of item 1475 wherein the anti-scarring agent is released in effective concentrations from the medical device at a decreasing rate.

1680. The medical device of item 1475 wherein the anti-scarring agent is released in effective concentrations from the composition comprising the anti-scarring agent by diffusion over a period ranging from the time of deployment of the medical device to about 90 days.

1681. The medical device of item 1475 wherein the anti-scarring agent is released in effective concentrations from the composition comprising the anti-scarring agent by erosion of the composition over a period ranging from the time of deployment of the medical device to about 90 days.

1682. The medical device of item 1475 wherein the device comprises about 0.01 μg to about 10 μg of the anti-scarring agent.

1683. The medical device of item 1475 wherein the device comprises about 10 μg to about 10 mg of the anti-scarring agent.

1684. The medical device of item 1475 wherein the device comprises about 10 mg to about 250 mg of the anti-scarring agent.

1685. The medical device of item 1475 wherein the device comprises about 250 mg to about 1000 mg of the anti-scarring agent.

1686. The medical device of item 1475 wherein the device comprises about 1000 mg to about 2500 mg of the anti-scarring agent.

1687. The medical device of item 1475 wherein a surface of the device comprises less than 0.01 μg of the anti-scarring agent per mm² of device surface to which the anti-scarring agent is applied.

1688. The medical device of item 1475 wherein a surface of the device comprises about 0.01 μg to about 1 μg of the anti-scarring agent per mm² of device surface to which the anti-scarring agent is applied.

1689. The medical device of item 1475 wherein a surface of the device comprises about 1 μg to about 10 μg of the anti-scarring agent per mm² of device surface to which the anti-scarring agent is applied.

1690. The medical device of item 1475 wherein a surface of the device comprises about 10 μg to about 250 μg of the anti-scarring agent per mm² of device surface to which the anti-scarring agent is applied.

1691. The medical device of item 1475 wherein a surface of the device comprises about 250 μg to about 1000 μg of the anti-scarring agent of anti-scarring agent per mm² of device surface to which the anti-scarring agent is applied.

1692. The medical device of item 1475 wherein a surface of the device comprises about 1000 μg to about 2500 μg of the anti-scarring agent per mm² of device surface to which the anti-scarring agent is applied.

1693. The medical device of item 1475 wherein the agent or the composition is affixed to the electrical device.

1694. The medical device of item 1475 wherein the agent or the composition is covalently attached to the electrical device.

1695. The medical device of item 1475 wherein the agent or the composition is non-covalently attached to the electrical device.

1696. The medical device of item 1475 further comprising a coating that absorbs the agent or the composition.

1697. The medical device of item 1475 wherein the electrical device is interweaved with a thread composed of, or coated with, the agent or the composition.

1698. The medical device of item 1475 wherein a portion of the electrical device is covered with a sleeve that contains the agent or the composition.

1699. The medical device of item 1475 wherein the electrical device is completely covered with a sleeve that contains the agent or the composition.

1700. The medical device of item 1475 wherein a portion of the electrical device is covered with a mesh that contains the agent or the composition.

1701. The medical device of item 1475 wherein the electrical device is completely covered with a mesh that contains the agent or the composition.

1702. The medical device of any one of items 1475-1701 wherein the gastric nerve stimulator is adapted for treating or preventing morbid obesity.

1703. The medical device of any one of items 1475-1701 wherein the gastric nerve stimulator is adapted for treating or preventing constipation.

1704. The medical device of any one of items 1475-1701 wherein the gastric nerve stimulator comprises an electrical lead, an electrode and a stimulation generator.

1705. The medical device of any one of items 1475-1701 wherein the gastric nerve stimulator comprises an electrical signal controller, connector wire and an attachment lead.

1706. A medical device, comprising a cochlear implant for treating deafness (i.e., an electrical device) and an anti-scarring agent or a composition comprising an anti-scarring agent, wherein the agent inhibits scarring between the medical device and the host into which the medical device is implanted.

1707. The medical device of item 1706 wherein the agent is an adensosine A2A receptor antagonist.

1708. The medical device of item 1706 wherein the agent is an AKT inhibitor.

1709. The medical device of item 1706 wherein the agent is an alpha 2 integrin antagonist, wherein the alpha 2 integrin antagonist is Pharmaprojects No. 5754 (Merck KgaA).

1710. The medical device of item 1706 wherein the agent is an alpha 4 integrin antagonist.

1711. The medical device of item 1706 wherein the agent is an alpha 7 nicotinic receptor agonist.

1712. The medical device of item 1706 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Aftenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

1713. The medical device of item 1706 wherein the agent is an apoptosis antagonist.

1714. The medical device of item 1706 wherein the agent is an apoptosis activator.

1715. The medical device of item 1706 wherein the agent is a beta 1 integrin antagonist.

1716. The medical device of item 1706 wherein the agent is a beta tubulin inhibitor.

1717. The medical device of item 1706 wherein the agent is a blocker of enzyme production in Hepatitis C

1718. The medical device of item 1706 wherein the agent is a Bruton's tyrosine kinase inhibitor.

1719. The medical device of item 1706 wherein the agent is a calcineurin inhibitor.

1720. The medical device of item 1706 wherein the agent is a caspase 3 inhibitor.

1721. The medical device of item 1706 wherein the agent is a CC chemokine receptor antagonist.

1722. The medical device of item 1706 wherein the agent is a cell cycle inhibitor selected from the group consisting of SNS-595 (Sunesis), synthadotin, KRX-0403, and an analogue or derivative thereof.

1723. The medical device of item 1706 wherein the agent is a cathepsin B inhibitor.

1724. The medical device of item 1706 wherein the agent is a cathepsin K inhibitor, wherein the cathepsin K inhibitor is 462795 (GlaxoSmithKline), INPL-022-D6 (Amura Therapeutics), or an analogue or derivative thereof.

1725. The medical device of item 1706 wherein the agent is a cathepsin L inhibitor.

1726. The medical device of item 1706 wherein the agent is a CD40 antagonist.

1727. The medical device of item 1706 wherein the agent is a chemokine receptor agonist.

1728. The medical device of item 1706 wherein the agent is a chymase inhibitor.

1729. The medical device of item 1706 wherein the agent is a collagenase antagonist.

1730. The medical device of item 1706 wherein the agent is a CXCR antagonist.

1731. The medical device of item 1706 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

1732. The medical device of item 1706 wherein the agent is a cyclooxygenase 1 inhibitor.

1733. The medical device of item 1706 wherein the agent is a DHFR inhibitor.

1734. The medical device of item 1706 wherein the agent is a dual integrin inhibitor.

1735. The medical device of item 1706 wherein the agent is an elastase inhibitor.

1736. The medical device of item 1706 wherein the agent is an elongation factor-1 alpha inhibitor.

1737. The medical device of item 1706 wherein the agent is an endothelial growth factor antagonist.

1738. The medical device of item 1706 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

1739. The medical device of item 1706 wherein the agent is an endotoxin antagonist.

1740. The medical device of item 1706 wherein the agent is an epothilone and tubulin binder.

1741. The medical device of item 1706 wherein the agent is an estrogen receptor antagonist.

1742. The medical device of item 1706 wherein the agent is an FGF inhibitor.

1743. The medical device of item 1706 wherein the agent is a farnexyl transferase inhibitor.

1744. The medical device of item 1706 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

1745. The medical device of item 1706 wherein the agent is an FLT-3 kinase inhibitor.

1746. The medical device of item 1706 wherein the agent is an FGF receptor kinase inhibitor.

1747. The medical device of item 1706 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

1748. The medical device of item 1706 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

1749. The medical device of item 1706 wherein the agent is a histone deacetylase inhibitor.

1750. The medical device of item 1706 wherein the agent is an HMGCoA reductase inhibitor selected from the group consisting of an atherosclerosis therapeutic from Lipid Sciences, ATI-16000 (ARYx

Therapeutics), KS-01-019 (Kos Pharmaceuticals), Pharmaprojects No. 2197 (Sanofi-Aventi), RP 61969 (Sanofi-Aventis), and an analogue or derivative thereof.

1751. The medical device of item 1706 wherein the agent is an ICAM inhibitor.

1752. The medical device of item 1706 wherein the agent is an IL, ICE and IRAK antagonist, wherein the antagonist is a CJ-14877, CP-424174 (Pfizer), NF-61 (Negma-Lerads), and an analogue or derivative thereof.

1753. The medical device of item 1706 wherein the agent is an IL-2 inhibitor.

1754. The medical device of item 1706 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

1755. The medical device of item 1706 wherein the agent is an IMPDH (inosine monophosphate).

1756. The medical device of item 1706 wherein the agent is an integrin antagonist.

1757. The medical device of item 1706 wherein the agent is an interleukin antagonist.

1758. The medical device of item 1706 wherein the agent is an inhibitor of type III receptor tyrosine kinase.

1759. The medical device of item 1706 wherein the agent is an irreversible inhibitor of enzyme methionine aminopeptidase type 2.

1760. The medical device of item 1706 wherein the agent is an isozyme selective delta protein kinase C inhibitor.

1761. The medical device of item 1706 wherein the agent a JAK3 enzyme inhibitor.

1762. The medical device of item 1706 wherein the agent is a JNK inhibitor.

1763. The medical device of item 1706 wherein the agent is a kinase inhibitor.

1764. The medical device of item 1706 wherein the agent is kinesin antagonist.

1765. The medical device of item 1706 wherein the agent is a kinesin antagonist.

1766. The medical device of item 1706 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

1767. The medical device of item 1706 wherein the agent is an MAP kinase inhibitor.

1768. The medical device of item 1706 wherein the agent is a matrix metalloproteinase inhibitor.

1769. The medical device of item 1706 wherein the agent is an MCP-CCR2 inhibitor.

1770. The medical device of item 1706 wherein the agent is an mTOR inhibitor.

1771. The medical device of item 1706 wherein the agent is an mTOR kinase inhibitor.

1772. The medical device of item 1706 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

1773. The medical device of item 1706 wherein the agent is an MIF inhibitor.

1774. The medical device of item 1706 wherein the agent is an MMP inhibitor.

1775. The medical device of item 1706 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

1776. The medical device of item 1706 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

1777. The medical device of item 1706 wherein the agent is a nitric oxide agonist.

1778. The medical device of item 1706 wherein the agent is an ornithine decarboxylase inhibitor.

1779. The medical device of item 1706 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

1780. The medical device of item 1706 wherein the agent is a palmitoyl-protein thioesterase inhibitor.

1781. The medical device of item 1706 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

1782. The medical device of item 1706 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-11 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

1783. The medical device of item 1706 wherein the agent is a phosphatase inhibitor.

1784. The medical device of item 1706 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

1785. The medical device of item 1706 wherein the agent is a PKC inhibitor.

1786. The medical device of item 1706 wherein the agent is a platelet activating factor antagonist.

1787. The medical device of item 1706 wherein the agent is a platelet-derived growth factor receptor kinase inhibitor.

1788. The medical device of item 1706 wherein the agent is a prolyl hydroxylase inhibitor.

1789. The medical device of item 1706 wherein the agent is a polymorphonuclear neutrophil inhibitor.

1790. The medical device of item 1706 wherein the agent is a protein kinase B inhibitor.

1791. The medical device of item 1706 wherein the agent is a protein kinase C stimulant.

1792. The medical device of item 1706 wherein the agent is a purine nucleoside analogue.

1793. The medical device of item 1706 wherein the agent is a purinoreceptor P2X antagonist.

1794. The medical device of item 1706 wherein the agent is a Raf kinase inhibitor.

1795. The medical device of item 1706 wherein the agent is a reversible inhibitor of ErbB1 and ErbB2.

1796. The medical device of item 1706 wherein the agent is a ribonucleoside triphosphate reductase inhibitor.

1797. The medical device of item 1706 wherein the agent is an SDF-1 antagonist.

1798. The medical device of item 1706 wherein the agent is a sheddase inhibitor.

1799. The medical device of item 1706 wherein the agent is an SRC inhibitor.

1800. The medical device of item 1706 wherein the agent is a stromelysin inhibitor.

1801. The medical device of item 1706 wherein the agent is an Syk kinase inhibitor.

1802. The medical device of item 1706 wherein the agent is a telomerase inhibitor.

1803. The medical device of item 1706 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

1804. The medical device of item 1706 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VF-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

1805. The medical device of item 1706 wherein the agent is a Toll receptor inhibitor

1806. The medical device of item 1706 wherein the agent is a tubulin antagonist.

1807. The medical device of item 1706 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (GAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

1808. The medical device of item 1706 wherein the agent is a VEGF inhibitor.

1809. The medical device of item 1706 wherein the agent is a vitamin D receptor agonist.

1810. The medical device of item 1706 wherein the agent is ZD-6474 (an angiogenesis inhibitor).

1811. The medical device of item 1706 wherein the agent is AP-23573 (an mTOR inhibitor).

1812. The medical device of item 1706 wherein the agent is synthadotin (a tubulin antagonist).

1813. The medical device of item 1706 wherein the agent is S-0885 (a collagenase inhibitor).

1814. The medical device of item 1706 wherein the agent is aplidine (an elongation factor-1 alpha inhibitor).

1815. The medical device of item 1706 wherein the agent is ixabepilone (an epithilone).

1816. The medical device of item 1706 wherein the agent is IDN-5390 (an angiogenesis inhibitor).

1817. The medical device of item 1706 wherein the agent is SB-2723005 (an angiogenesis inhibitor).

1818. The medical device of item 1706 wherein the agent is ABT-518 (an angiogenesis inhibitor).

1819. The medical device of item 1706 wherein the agent is combretastatin (an angiogenesis inhibitor).

1820. The medical device of item 1706 wherein the agent is anecortave acetate (an angiogenesis inhibitor).

1821. The medical device of item 1706 wherein the agent is SB-715992 (a kinesin antagonist).

1822. The medical device of item 1706 wherein the agent is temsirolimus (an mTOR inhibitor).

1823. The medical device of item 1706 wherein the agent is adalimumab (a TNFα antagonist).

1824. The medical device of item 1706, further comprising a coating, wherein the coating comprises the anti-scarring agent and a polymer.

1825. The medical device of item 1706, further comprising a coating, wherein the coating comprises the anti-scarring agent.

1826. The medical device of item 1706, further comprising a coating, wherein the coating is disposed on a surface of the electrical device.

1827. The medical device of item 1706, further comprising a coating, wherein the coating directly contacts the electrical device.

1828. The medical device of item 1706, further comprising a coating, wherein the coating indirectly contacts the electrical device.

1829. The medical device of item 1706, further comprising a coating, wherein the coating partially covers the electrical device.

1830. The medical device of item 1706, further comprising a coating, wherein the coating completely covers the electrical device.

1831. The medical device of item 1706, further comprising a coating, wherein the coating is a uniform coating.

1832. The medical device of item 1706, further comprising a coating, wherein the coating is a non-uniform coating.

1833. The medical device of item 1706, further comprising a coating, wherein the coating is a discontinuous coating.

1834. The medical device of item 1706, further comprising a coating, wherein the coating is a patterned coating.

1835. The medical device of item 1706, further comprising a coating, wherein the coating has a thickness of 100 μm or less.

1836. The medical device of item 1706, further comprising a coating, wherein the coating has a thickness of 10 μm or less.

1837. The medical device of item 1706, further comprising a coating, wherein the coating adheres to the surface of the electrical device upon deployment of the medical device.

1838. The medical device of item 1706, further comprising a coating, wherein the coating is stable at room temperature for a period of 1 year.

1839. The medical device of item 1706, further comprising a coating, wherein the anti-scarring agent is present in the coating in an amount ranging between about 0.0001% to about 1% by weight.

1840. The medical device of item 1706, further comprising a coating, wherein the anti-scarring agent is present in the coating in an amount ranging between about 1% to about 10% by weight.

1841. The medical device of item 1706, further comprising a coating, wherein the anti-scarring agent is present in the coating in an amount ranging between about 10% to about 25% by weight.

1842. The medical device of item 1706, further comprising a coating, wherein the anti-scarring agent is present in the coating in an amount ranging between about 25% to about 70% by weight.

1843. The medical device of item 1706, further comprising a coating, wherein the coating further comprises a polymer.

1844. The medical device of item 1706, further comprising a first coating having a first composition and the second coating having a second composition.

1845. The medical device of item 1706, further comprising a first coating having a first composition and the second coating having a second composition, wherein the first composition and the second composition are different.

1846. The medical device of item 1706, further comprising a polymer.

1847. The medical device of item 1706, further comprising a polymeric carrier.

1848. The medical device of item 1706, further comprising a polymeric carrier, wherein the polymeric carrier comprises a copolymer.

1849. The medical device of item 1706, further comprising a polymeric carrier, wherein the polymeric carrier comprises a block copolymer.

1850. The medical device of item 1706, further comprising a polymeric carrier, wherein the polymeric carrier comprises a random copolymer.

1851. The medical device of item 1706, further comprising a polymeric carrier, wherein the polymeric carrier comprises a biodegradable polymer.

1852. The medical device of item 1706, further comprising a polymeric carrier, wherein the polymeric carrier comprises a non-biodegradable polymer.

1853. The medical device of item 1706, further comprising a polymeric carrier, wherein the polymeric carrier comprises a hydrophilic polymer.

1854. The medical device of item 1706, further comprising a polymeric carrier, wherein the polymeric carrier comprises a hydrophobic polymer.

1855. The medical device of item 1706, further comprising a polymeric carrier, wherein the polymeric carrier comprises a polymer having hydrophilic domains.

1856. The medical device of item 1706, further comprising a polymeric carrier, wherein the polymeric carrier comprises a polymer having hydrophobic domains.

1857. The medical device of item 1706, further comprising a polymeric carrier, wherein the polymeric carrier comprises a non-conductive polymer.

1858. The medical device of item 1706, further comprising a polymeric carrier, wherein the polymeric carrier comprises an elastomer.

1859. The medical device of item 1706, further comprising a polymeric carrier, wherein the polymeric carrier comprises a hydrogel.

1860. The medical device of item 1706, further comprising a polymeric carrier, wherein the polymeric carrier comprises a silicone polymer.

1861. The medical device of item 1706, further comprising a polymeric carrier, wherein the polymeric carrier comprises a hydrocarbon polymer.

1862. The medical device of item 1706, further comprising a polymeric carrier, wherein the polymeric carrier comprises a styrene-derived polymer.

1863. The medical device of item 1706, further comprising a polymeric carrier, wherein the polymeric carrier comprises a butadiene polymer.

1864. The medical device of item 1706, further comprising a polymeric carrier, wherein the polymeric carrier comprises a macromer.

1865. The medical device of item 1706, further comprising a polymeric carrier, wherein the polymeric carrier comprises a poly(ethylene glycol) polymer.

1866. The medical device of item 1706, further comprising a polymeric carrier, wherein the polymeric carrier comprises an amorphous polymer.

1867. The medical device of item 1706, further comprising a lubricious coating.

1868. The medical device of item 1706 wherein the anti-scarring agent is located within pores or holes of the electrical device.

1869. The medical device of item 1706 wherein the anti-scarring agent is located within a channel, lumen, or divet of the electrical device.

1870. The medical device of item 1706, further comprising a second pharmaceutically active agent.

1871. The medical device of item 1706, further comprising an anti-inflammatory agent.

1872. The medical device of item 1706, further comprising an agent that inhibits infection.

1873. The medical device of item 1706, further comprising an agent that inhibits infection, wherein the agent is an anthracycline.

1874. The medical device of item 1706, further comprising an agent that inhibits infection, wherein the agent is doxorubicin.

1875. The medical device of item 1706, further comprising an agent that inhibits infection, wherein the agent is mitoxantrone.

1876. The medical device of item 1706, further comprising an agent that inhibits infection, wherein the agent is a fluoropyrimidine.

1877. The medical device of item 1706, further comprising an agent that inhibits infection, wherein the agent is 5-fluorouracil (5-FU).

1878. The medical device of item 1706, further comprising an agent that inhibits infection, wherein the agent is a folic acid antagonist.

1879. The medical device of item 1706, further comprising an agent that inhibits infection, wherein the agent is methotrexate.

1880. The medical device of item 1706, further comprising an agent that inhibits infection, wherein the agent is a podophylotoxin.

1881. The medical device of item 1706, further comprising an agent that inhibits infection, wherein the agent is etoposide.

1882. The medical device of item 1706, further comprising an agent that inhibits infection, wherein the agent is a camptothecin.

1883. The medical device of item 1706, further comprising an agent that inhibits infection, wherein the agent is a hydroxyurea.

1884. The medical device of item 1706, further comprising an agent that inhibits infection, wherein the agent is a platinum complex.

1885. The medical device of item 1706, further comprising an agent that inhibits infection, wherein the agent is cisplatin.

1886. The medical device of item 1706, further comprising an anti-thrombotic agent.

1887. The medical device of item 1706, further comprising a visualization agent.

1888. The medical device of item 1706, further comprising a visualization agent, wherein the visualization agent is a radiopaque material, wherein the radiopaque material comprises a metal, a halogenated compound, or a barium containing compound.

1889. The medical device of item 1706, further comprising a visualization agent, wherein the visualization agent is a radiopaque material, wherein the radiopaque material comprises barium, tantalum, or technetium.

1890. The medical device of item 1706, further comprising a visualization agent, wherein the visualization agent is a MRI responsive material.

1891. The medical device of item 1706, further comprising a visualization agent, wherein the visualization agent comprises a gadolinium chelate.

1892. The medical device of item 1706, further comprising a visualization agent, wherein the visualization agent comprises iron, magnesium, manganese, copper, or chromium.

1893. The medical device of item 1706, further comprising a visualization agent, wherein the visualization agent comprises an iron oxide compound.

1894. The medical device of item 1706, further comprising a visualization agent, wherein the visualization agent comprises a dye, pigment, or colorant.

1895. The medical device of item 1706, further comprising an echogenic material.

1896. The medical device of item 1706, further comprising an echogenic material, wherein the echogenic material is in the form of a coating.

1897. The medical device of item 1706 wherein the device is sterile.

1898. The medical device of item 1706 wherein the anti-scarring agent inhibits adhesion between the medical device and a host into which the medical device is implanted.

1899. The medical device of item 1706 wherein the medical device delivers the anti-scarring agent locally to tissue proximate to the medical device.

1900. The medical device of item 1706 wherein the anti-scarring agent is released into tissue in the vicinity of the medical device after deployment of the medical device.

1901. The medical device of item 1706 wherein the anti-scarring agent is released into tissue in the vicinity of the medical device after deployment of the medical device, wherein the tissue is connective tissue.

1902. The medical device of item 1706 wherein the anti-scarring agent is released into tissue in the vicinity of the medical device after deployment of the medical device, wherein the tissue is muscle tissue.

1903. The medical device of item 1706 wherein the anti-scarring agent is released into tissue in the vicinity of the medical device after deployment of the medical device, wherein the tissue is nerve tissue.

1904. The medical device of item 1706 wherein the anti-scarring agent is released into tissue in the vicinity of the medical device after deployment of the medical device, wherein the tissue is epithelium tissue.

1905. The medical device of item 1706 wherein the anti-scarring agent is released in effective concentrations from the medical device over a period ranging from the time of deployment of the medical device to about 1 year.

1906. The medical device of item 1706 wherein the anti-scarring agent is released in effective concentrations from the medical device over a period ranging from about 1 month to 6 months.

1907. The medical device of item 1706 wherein the anti-scarring agent is released in effective concentrations from the medical device over a period ranging from about 1-90 days.

1908. The medical device of item 1706 wherein the anti-scarring agent is released in effective concentrations from the medical device at a constant rate.

1909. The medical device of item 1706 wherein the anti-scarring agent is released in effective concentrations from the medical device at an increasing rate.

1910. The medical device of item 1706 wherein the anti-scarring agent is released in effective concentrations from the medical device at a decreasing rate.

1911. The medical device of item 1706 wherein the anti-scarring agent is released in effective concentrations from the composition comprising the anti-scarring agent by diffusion over a period ranging from the time of deployment of the medical device to about 90 days.

1912. The medical device of item 1706 wherein the anti-scarring agent is released in effective concentrations from the composition comprising the anti-scarring agent by erosion of the composition over a period ranging from the time of deployment of the medical device to about 90 days.

1913. The medical device of item 1706 wherein the device comprises about 0.01 μg to about 10 μg of the anti-scarring agent.

1914. The medical device of item 1706 wherein the device comprises about 10 μg to about 10 mg of the anti-scarring agent.

1915. The medical device of item 1706 wherein the device comprises about 10 mg to about 250 mg of the anti-scarring agent.

1916. The medical device of item 1706 wherein the device comprises about 250 mg to about 1000 mg of the anti-scarring agent.

1917. The medical device of item 1706 wherein the device comprises about 1000 mg to about 2500 mg of the anti-scarring agent.

1918. The medical device of item 1706 wherein a surface of the device comprises less than 0.01 μg of the anti-scarring agent per mm² of device surface to which the anti-scarring agent is applied.

1919. The medical device of item 1706 wherein a surface of the device comprises about 0.01 μg to about 1 μg of the anti-scarring agent per mm² of device surface to which the anti-scarring agent is applied.

1920. The medical device of item 1706 wherein a surface of the device comprises about 1 μg to about 10 μg of the anti-scarring agent per mm² of device surface to which the anti-scarring agent is applied.

1921. The medical device of item 1706 wherein a surface of the device comprises about 10 μg to about 250 μg of the anti-scarring agent per mm² of device surface to which the anti-scarring agent is applied.

1922. The medical device of item 1706 wherein a surface of the device comprises about 250 μg to about 1000 μg of the anti-scarring agent of anti-scarring agent per mm² of device surface to which the anti-scarring agent is applied.

1923. The medical device of item 1706 wherein a surface of the device comprises about 1000 μg to about 2500 μg of the anti-scarring agent per mm² of device surface to which the anti-scarring agent is applied.

1924. The medical device of item 1706 wherein the agent or the composition is affixed to the electrical device.

1925. The medical device of item 1706 wherein the agent or the composition is covalently attached to the electrical device.

1926. The medical device of item 1706 wherein the agent or the composition is non-covalently attached to the electrical device.

1927. The medical device of item 1706 further comprising a coating that absorbs the agent or the composition.

1928. The medical device of item 1706 wherein the electrical device is interweaved with a thread composed of, or coated with, the agent or the composition.

1929. The medical device of item 1706 wherein a portion of the electrical device is covered with a sleeve that contains the agent or the composition.

1930. The medical device of item 1706 wherein the electrical device is completely covered with a sleeve that contains the agent or the composition.

1931. The medical device of item 1706 wherein a portion of the electrical device is covered with a mesh that contains the agent or the composition.

1932. The medical device of item 1706 wherein the electrical device is completely covered with a mesh that contains the agent or the composition.

1933. The medical device of any one of items 1706-1932 wherein the cochlear implant comprises a plurality of transducer elements.

1934. The medical device of any one of items 1706-1932 wherein the cochlear implant comprises a sound-to-electrical stimulation encoder, a body implantable receiver-stimulator, and electrodes.

1935. The medical device of any one of items 1706-1932 wherein the cochlear implant comprises a transducer and an electrode array.

1936. The medical device of any one of items 1706-1932 wherein the cochlear implant is a subcranially implantable electomechanical system.

1937. A medical device, comprising a bone growth stimulator (i.e., an electrical device) and an anti-scarring agent or a composition comprising an anti-scarring agent, wherein the agent inhibits scarring between the medical device and the host into which the medical device is implanted.

1938. The medical device of item 1937 wherein the agent is an adensosine A2A receptor antagonist.

1939. The medical device of item 1937 wherein the agent is an AKT inhibitor.

1940. The medical device of item 1937 wherein the agent is an alpha 2 integrin antagonist, wherein the alpha 2 integrin antagonist is Pharmaprojects No. 5754 (Merck KgaA).

1941. The medical device of item 1937 wherein the agent is an alpha 4 integrin antagonist.

1942. The medical device of item 1937 wherein the agent is an alpha 7 nicotinic receptor agonist.

1943. The medical device of item 1937 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

1944. The medical device of item 1937 wherein the agent is an apoptosis antagonist.

1945. The medical device of item 1937 wherein the agent is an apoptosis activator.

1946. The medical device of item 1937 wherein the agent is a beta 1 integrin antagonist.

1947. The medical device of item 1937 wherein the agent is a beta tubulin inhibitor.

1948. The medical device of item 1937 wherein the agent is a blocker of enzyme production in Hepatitis C.

1949. The medical device of item 1937 wherein the agent is a Bruton's tyrosine kinase inhibitor.

1950. The medical device of item 1937 wherein the agent is a calcineurin inhibitor.

1951. The medical device of item 1937 wherein the agent is a caspase 3 inhibitor.

1952. The medical device of item 1937 wherein the agent is a CC chemokine receptor antagonist.

1953. The medical device of item 1937 wherein the agent is a cell cycle inhibitor selected from the group consisting of SNS-595 (Sunesis), synthadotin, KRX-0403, and an analogue or derivative thereof.

1954. The medical device of item 1937 wherein the agent is a cathepsin B inhibitor.

1955. The medical device of item 1937 wherein the agent is a cathepsin K inhibitor, wherein the cathepsin K inhibitor is 462795 (GlaxoSmithKIine), INPL-022-D6 (Amura Therapeutics), or an analogue or derivative thereof.

1956. The medical device of item 1937 wherein the agent is a cathepsin L inhibitor.

1957. The medical device of item 1937 wherein the agent is a CD40 antagonist.

1958. The medical device of item 1937 wherein the agent is a chemokine receptor agonist.

1959. The medical device of item 1937 wherein the agent is a chymase inhibitor.

1960. The medical device of item 1937 wherein the agent is a collagenase antagonist.

1961. The medical device of item 1937 wherein the agent is a CXCR antagonist.

1962. The medical device of item 1937 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

1963. The medical device of item 1937 wherein the agent is a cyclooxygenase 1 inhibitor.

1964. The medical device of item 1937 wherein the agent is a DHFR inhibitor.

1965. The medical device of item 1937 wherein the agent is a dual integrin inhibitor.

1966. The medical device of item 1937 wherein the agent is an elastase inhibitor.

1967. The medical device of item 1937 wherein the agent is an elongation factor-1 alpha inhibitor.

1968. The medical device of item 1937 wherein the agent is an endothelial growth factor antagonist.

1969. The medical device of item 1937 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

1970. The medical device of item 1937 wherein the agent is an endotoxin antagonist.

1971. The medical device of item 1937 wherein the agent is an epothilone and tubulin binder.

1972. The medical device of item 1937 wherein the agent is an estrogen receptor antagonist.

1973. The medical device of item 1937 wherein the agent is an FGF inhibitor.

1974. The medical device of item 1937 wherein the agent is a farnexyl transferase inhibitor.

1975. The medical device of item 1937 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

1976. The medical device of item 1937 wherein the agent is an FLT-3 kinase inhibitor.

1977. The medical device of item 1937 wherein the agent is an FGF receptor kinase inhibitor.

1978. The medical device of item 1937 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

1979. The medical device of item 1937 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamyci n XIV, 1′,4-didehydro-1-deoxy-1,4-dihydro-5′-(2-methylpropyl)-1-oxo-), and an analogue or derivative thereof.

1980. The medical device of item 1937 wherein the agent is a histone deacetylase inhibitor.

1981. The medical device of item 1937 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

1982. The medical device of item 1937 wherein the agent is an ICAM inhibitor.

1983. The medical device of item 1937 wherein the agent is an IL, ICE and IRAK antagonist, wherein the antagonist is a CJ-14877, CP-424174 (Pfizer), NF-61 (Negma-Lerads), and an analogue or derivative thereof.

1984. The medical device of item 1937 wherein the agent is an IL-2 inhibitor.

1985. The medical device of item 1937 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 11878841-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

1986. The medical device of item 1937 wherein the agent is an IMPDH (inosine monophosphate).

1987. The medical device of item 1937 wherein the agent is an integrin antagonist.

1988. The medical device of item 1937 wherein the agent is an interleukin antagonist.

1989. The medical device of item 1937 wherein the agent is an inhibitor of type III receptor tyrosine kinase.

1990. The medical device of item 1937 wherein the agent is an irreversible inhibitor of enzyme methionine aminopeptidase type 2.

1991. The medical device of item 1937 wherein the agent is an isozyme selective delta protein kinase C inhibitor.

1992. The medical device of item 1937 wherein the agent a JAK3 enzyme inhibitor.

1993. The medical device of item 1937 wherein the agent is a JNK inhibitor.

1994. The medical device of item 1937 wherein the agent is a kinase inhibitor.

1995. The medical device of item 1937 wherein the agent is kinesin antagonist.

1996. The medical device of item 1937 wherein the agent is a kinesin antagonist.

1997. The medical device of item 1937 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

1998. The medical device of item 1937 wherein the agent is an MAP kinase inhibitor.

1999. The medical device of item 1937 wherein the agent is a matrix metalloproteinase inhibitor.

2000. The medical device of item 1937 wherein the agent is an MCP-CCR2 inhibitor.

2001. The medical device of item 1937 wherein the agent is an mTOR inhibitor.

2002. The medical device of item 1937 wherein the agent is an mTOR kinase inhibitor.

2003. The medical device of item 1937 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

2004. The medical device of item 1937 wherein the agent is an MIF inhibitor.

2005. The medical device of item 1937 wherein the agent is an MMP inhibitor.

2006. The medical device of item 1937 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

2007. The medical device of item 1937 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

2008. The medical device of item 1937 wherein the agent is a nitric oxide agonist.

2009. The medical device of item 1937 wherein the agent is an ornithine decarboxylase inhibitor.

2010. The medical device of item 1937 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

2011. The medical device of item 1937 wherein the agent is a palmitoyl-protein thioesterase inhibitor.

2012. The medical device of item 1937 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

2013. The medical device of item 1937 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

2014. The medical device of item 1937 wherein the agent is a phosphatase inhibitor.

2015. The medical device of item 1937 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

2016. The medical device of item 1937 wherein the agent is a PKC inhibitor.

2017. The medical device of item 1937 wherein the agent is a platelet activating factor antagonist.

2018. The medical device of item 1937 wherein the agent is a platelet-derived growth factor receptor kinase inhibitor.

2019. The medical device of item 1937 wherein the agent is a prolyl hydroxylase inhibitor.

2020. The medical device of item 1937 wherein the agent is a polymorphonuclear neutrophil inhibitor.

2021. The medical device of item 1937 wherein the agent is a protein kinase B inhibitor.

2022. The medical device of item 1937 wherein the agent is a protein kinase C stimulant.

2023. The medical device of item 1937 wherein the agent is a purine nucleoside analogue.

2024. The medical device of item 1937 wherein the agent is a purinoreceptor P2X antagonist

2025. The medical device of item 1937 wherein the agent is a Raf kinase inhibitor.

2026. The medical device of item 1937 wherein the agent is a reversible inhibitor of ErbB1 and ErbB2.

2027. The medical device of item 1937 wherein the agent is a ribonucleoside triphosphate reductase inhibitor.

2028. The medical device of item 1937 wherein the agent is an SDF-1 antagonist.

2029. The medical device of item 1937 wherein the agent is a sheddase inhibitor.

2030. The medical device of item 1937 wherein the agent is an SRC inhibitor.

2031. The medical device of item 1937 wherein the agent is a stromelysin inhibitor.

2032. The medical device of item 1937 wherein the agent is an Syk kinase inhibitor.

2033. The medical device of item 1937 wherein the agent is a telomerase inhibitor.

2034. The medical device of item 1937 wherein the agent is a TGF beta inhibitor selected from the group consisting of pirfenidone (CAS No 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

2035. The medical device of item 1937 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

2036. The medical device of item 1937 wherein the agent is a Toll receptor inhibitor.

2037. The medical device of item 1937 wherein the agent is a tubulin antagonist.

2038. The medical device of item 1937 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-1 3 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

2039. The medical device of item 1937 wherein the agent is a VEGF inhibitor.

2040. The medical device of item 1937 wherein the agent is a vitamin D receptor agonist.

2041. The medical device of item 1937 wherein the agent is ZD-6474 (an angiogenesis inhibitor).

2042. The medical device of item 1937 wherein the agent is AP-23573 (an mTOR inhibitor).

2043. The medical device of item 1937 wherein the agent is synthadotin (a tubulin antagonist).

2044. The medical device of item 1937 wherein the agent is S-0885 (a collagenase inhibitor).

2045. The medical device of item 1937 wherein the agent is aplidine (an elongation factor-1 alpha inhibitor).

2046. The medical device of item 1937 wherein the agent is ixabepilone (an epithilone).

2047. The medical device of item 1937 wherein the agent is IDN-5390 (an angiogenesis inhibitor).

2048. The medical device of item 1937 wherein the agent is SB-2723005 (an angiogenesis inhibitor).

2049. The medical device of item 1937 wherein the agent is ABT-518 (an angiogenesis inhibitor).

2050. The medical device of item 1937 wherein the agent is combretastatin (an angiogenesis inhibitor).

2051. The medical device of item 1937 wherein the agent is anecortave acetate (an angiogenesis inhibitor).

2052. The medical device of item 1937 wherein the agent is SB-715992 (a kinesin antagonist).

2053. The medical device of item 1937 wherein the agent is temsirolimus (an mTOR inhibitor).

2054. The medical device of item 1937 wherein the agent is adalimumab (a TNFα antagonist).

2055. The medical device of item 1937, further comprising a coating, wherein the coating comprises the anti-scarring agent and a polymer.

2056. The medical device of item 1937, further comprising a coating, wherein the coating comprises the anti-scarring agent.

2057. The medical device of item 1937, further comprising a coating, wherein the coating is disposed on a surface of the electrical device.

2058. The medical device of item 1937, further comprising a coating, wherein the coating directly contacts the electrical device.

2059. The medical device of item 1937, further comprising a coating, wherein the coating indirectly contacts the electrical device.

2060. The medical device of item 1937, further comprising a coating, wherein the coating partially covers the electrical device.

2061. The medical device of item 1937, further comprising a coating, wherein the coating completely covers the electrical device.

2062. The medical device of item 1937, further comprising a coating, wherein the coating is a uniform coating.

2063. The medical device of item 1937, further comprising a coating, wherein the coating is a non-uniform coating.

2064. The medical device of item 1937, further comprising a coating, wherein the coating is a discontinuous coating.

2065. The medical device of item 1937, further comprising a coating, wherein the coating is a patterned coating.

2066. The medical device of item 1937, further comprising a coating, wherein the coating has a thickness of 100 μm or less.

2067. The medical device of item 1937, further comprising a coating, wherein the coating has a thickness of 10 μm or less.

2068. The medical device of item 1937, further comprising a coating, wherein the coating adheres to the surface of the electrical device upon deployment of the medical device.

2069. The medical device of item 1937, further comprising a coating, wherein the coating is stable at room temperature for a period of 1 year.

2070. The medical device of item 1937, further comprising a coating, wherein the anti-scarring agent is present in the coating in an amount ranging between about 0.0001% to about 1% by weight.

2071. The medical device of item 1937, further comprising a coating, wherein the anti-scarring agent is present in the coating in an amount ranging between about 1% to about 10% by weight.

2072. The medical device of item 1937, further comprising a coating, wherein the anti-scarring agent is present in the coating in an amount ranging between about 10% to about 25% by weight.

2073. The medical device of item 1937, further comprising a coating, wherein the anti-scarring agent is present in the coating in an amount ranging between about 25% to about 70% by weight.

2074. The medical device of item 1937, further comprising a coating, wherein the coating further comprises a polymer.

2075. The medical device of item 1937, further comprising a first coating having a first composition and the second coating having a second composition.

2076. The medical device of item 1937, further comprising a first coating having a first composition and the second coating having a second composition, wherein the first composition and the second composition are different.

2077. The medical device of item 1937, further comprising a polymer.

2078. The medical device of item 1937, further comprising a polymeric carrier.

2079. The medical device of item 1937, further comprising a polymeric carrier, wherein the polymeric carrier comprises a copolymer.

2080. The medical device of item 1937, further comprising a polymeric carrier, wherein the polymeric carrier comprises a block copolymer.

2081. The medical device of item 1937, further comprising a polymeric carrier, wherein the polymeric carrier comprises a random copolymer.

2082. The medical device of item 1937, further comprising a polymeric carrier, wherein the polymeric carrier comprises a biodegradable polymer.

2083. The medical device of item 1937, further comprising a polymeric carrier, wherein the polymeric carrier comprises a non-biodegradable polymer.

2084. The medical device of item 1937, further comprising a polymeric carrier, wherein the polymeric carrier comprises a hydrophilic polymer.

2085. The medical device of item 1937, further comprising a polymeric carrier, wherein the polymeric carrier comprises a hydrophobic polymer.

2086. The medical device of item 1937, further comprising a polymeric carrier, wherein the polymeric carrier comprises a polymer having hydrophilic domains.

2087. The medical device of item 1937, further comprising a polymeric carrier, wherein the polymeric carrier comprises a polymer having hydrophobic domains.

2088. The medical device of item 1937, further comprising a polymeric carrier, wherein the polymeric carrier comprises a non-conductive polymer.

2089. The medical device of item 1937, further comprising a polymeric carrier, wherein the polymeric carrier comprises an elastomer.

2090. The medical device of item 1937, further comprising a polymeric carrier, wherein the polymeric carrier comprises a hydrogel.

2091. The medical device of item 1937, further comprising a polymeric carrier, wherein the polymeric carrier comprises a silicone polymer.

2092. The medical device of item 1937, further comprising a polymeric carrier, wherein the polymeric carrier comprises a hydrocarbon polymer.

2093. The medical device of item 1937, further comprising a polymeric carrier, wherein the polymeric carrier comprises a styrene-derived polymer.

2094. The medical device of item 1937, further comprising a polymeric carrier, wherein the polymeric carrier comprises a butadiene polymer.

2095. The medical device of item 1937, further comprising a polymeric carrier, wherein the polymeric carrier comprises a macromer.

2096. The medical device of item 1937, further comprising a polymeric carrier, wherein the polymeric carrier comprises a poly(ethylene glycol) polymer.

2097. The medical device of item 1937, further comprising a polymeric carrier, wherein the polymeric carrier comprises an amorphous polymer.

2098. The medical device of item 1937, further comprising a lubricious coating.

2099. The medical device of item 1937 wherein the anti-scarring agent is located within pores or holes of the electrical device.

2100. The medical device of item 1937 wherein the anti-scarring agent is located within a channel, lumen, or divet of the electrical device.

2101. The medical device of item 1937, further comprising a second pharmaceutically active agent.

2102. The medical device of item 1937, further comprising an anti-inflammatory agent.

2103. The medical device of item 1937, further comprising an agent that inhibits infection.

2104. The medical device of item 1937, further comprising an agent that inhibits infection, wherein the agent is an anthracycline.

2105. The medical device of item 1937, further comprising an agent that inhibits infection, wherein the agent is doxorubicin.

2106. The medical device of item 1937, further comprising an agent that inhibits infection, wherein the agent is mitoxantrone.

2107. The medical device of item 1937, further comprising an agent that inhibits infection, wherein the agent is a fluoropyrimidine.

2108. The medical device of item 1937, further comprising an agent that inhibits infection, wherein the agent is 5-fluorouracil (5-FU).

2109. The medical device of item 1937, further comprising an agent that inhibits infection, wherein the agent is a folic acid antagonist.

2110. The medical device of item 1937, further comprising an agent that inhibits infection, wherein the agent is methotrexate.

2111. The medical device of item 1937, further comprising an agent that inhibits infection, wherein the agent is a podophylotoxin.

2112. The medical device of item 1937, further comprising an agent that inhibits infection, wherein the agent is etoposide.

2113. The medical device of item 1937, further comprising an agent that inhibits infection, wherein the agent is a camptothecin.

2114. The medical device of item 1937,further comprising an agent that inhibits infection, wherein the agent is a hydroxyurea.

2115. The medical device of item 1937, further comprising an agent that inhibits infection, wherein the agent is a platinum complex.

2116. The medical device of item 1937, further comprising an agent that inhibits infection, wherein the agent is cisplatin.

2117. The medical device of item 1937, further comprising an anti-thrombotic agent.

2118. The medical device of item 1937, further comprising a visualization agent.

2119. The medical device of item 1937, further comprising a visualization agent, wherein the visualization agent is a radiopaque material, wherein the radiopaque material comprises a metal, a halogenated compound, or a barium containing compound.

2120. The medical device of item 1937, further comprising a visualization agent, wherein the visualization agent is a radiopaque material, wherein the radiopaque material comprises barium, tantalum, or technetium.

2121. The medical device of item 1937, further comprising a visualization agent, wherein the visualization agent is a MRI responsive material.

2122. The medical device of item 1937, further comprising a visualization agent, wherein the visualization agent comprises a gadolinium chelate.

2123. The medical device of item 1937, further comprising a visualization agent, wherein the visualization agent comprises iron, magnesium, manganese, copper, or chromium.

2124. The medical device of item 1937, further comprising a visualization agent, wherein the visualization agent comprises an iron oxide compound.

2125. The medical device of item 1937, further comprising a visualization agent, wherein the visualization agent comprises a dye, pigment, or colorant.

2126. The medical device of item 1937, further comprising an echogenic material.

2127. The medical device of item 1937, further comprising an echogenic material, wherein the echogenic material is in the form of a coating.

2128. The medical device of item 1937 wherein the device is sterile.

2129. The medical device of item 1937 wherein the anti-scarring agent inhibits adhesion between the medical device and a host into which the medical device is implanted.

2130. The medical device of item 1937 wherein the medical device delivers the anti-scarring agent locally to tissue proximate to the medical device.

2131. The medical device of item 1937 wherein the anti-scarring agent is released into tissue in the vicinity of the medical device after deployment of the medical device.

2132. The medical device of item 1937 wherein the anti-scarring agent is released into tissue in the vicinity of the medical device after deployment of the medical device, wherein the tissue is connective tissue.

2133. The medical device of item 1937 wherein the anti-scarring agent is released into tissue in the vicinity of the medical device after deployment of the medical device, wherein the tissue is muscle tissue.

2134. The medical device of item 1937 wherein the anti-scarring agent is released into tissue in the vicinity of the medical device after deployment of the medical device, wherein the tissue is nerve tissue.

2135. The medical device of item 1937 wherein the anti-scarring agent is released into tissue in the vicinity of the medical device after deployment of the medical device, wherein the tissue is epithelium tissue.

2136. The medical device of item 1937 wherein the anti-scarring agent is released in effective concentrations from the medical device over a period ranging from the time of deployment of the medical device to about 1 year.

2137. The medical device of item 1937 wherein the anti-scarring agent is released in effective concentrations from the medical device over a period ranging from about 1 month to 6 months.

2138. The medical device of item 1937 wherein the anti-scarring agent is released in effective concentrations from the medical device over a period ranging from about 1-90 days.

2139. The medical device of item 1937 wherein the anti-scarring agent is released in effective concentrations from the medical device at a constant rate.

2140. The medical device of item 1937 wherein the anti-scarring agent is released in effective concentrations from the medical device at an increasing rate.

2141. The medical device of item 1937 wherein the anti-scarring agent is released in effective concentrations from the medical device at a decreasing rate.

2142. The medical device of item 1937 wherein the anti-scarring agent is released in effective concentrations from the composition comprising the anti-scarring agent by diffusion over a period ranging from the time of deployment of the medical device to about 90 days.

2143. The medical device of item 1937 wherein the anti-scarring agent is released in effective concentrations from the composition comprising the anti-scarring agent by erosion of the composition over a period ranging from the time of deployment of the medical device to about 90 days.

2144. The medical device of item 1937 wherein the device comprises about 0.01 μg to about 10 μg of the anti-scarring agent.

2145. The medical device of item 1937 wherein the device comprises about 10 μg to about 10 mg of the anti-scarring agent.

2146. The medical device of item 1937 wherein the device comprises about 10 mg to about 250 mg of the anti-scarring agent.

2147. The medical device of item 1937 wherein the device comprises about 250 mg to about 1000 mg of the anti-scarring agent.

2148. The medical device of item 1937 wherein the device comprises about 1000 mg to about 2500 mg of the anti-scarring agent.

2149. The medical device of item 1937 wherein a surface of the device comprises less than 0.01 μg of the anti-scarring agent per mm² of device surface to which the anti-scarring agent is applied.

2150. The medical device of item 1937 wherein a surface of the device comprises about 0.01 μg to about 1 μg of the anti-scarring agent per mm² of device surface to which the anti-scarring agent is applied.

2151. The medical device of item 1937 wherein a surface of the device comprises about 1 μg to about 10 μg of the anti-scarring agent per mm² of device surface to which the anti-scarring agent is applied.

2152. The medical device of item 1937 wherein a surface of the device comprises about 10 μg to about 250 μg of the anti-scarring agent per mm² of device surface to which the anti-scarring agent is applied.

2153. The medical device of item 1937 wherein a surface of the device comprises about 250 μg to about 1000 μg of the anti-scarring agent of anti-scarring agent per mm² of device surface to which the anti-scarring agent is applied.

2154. The medical device of item 1937 wherein a surface of the device comprises about 1000 μg to about 25001g of the anti-scarring agent per mm² of device surface to which the anti-scarring agent is applied

2155. The medical device of item 1937 wherein the agent or the composition is affixed to the electrical device.

2156. The medical device of item 1937 wherein the agent or the composition is covalently attached to the electrical device.

2157. The medical device of item 1937 wherein the agent or the composition is non-covalently attached to the electrical device.

2158. The medical device of item 1937 further comprising a coating that absorbs the agent or the composition.

2159. The medical device of item 1937 wherein the electrical device is interweaved with a thread composed of, or coated with, the agent or the composition.

2160. The medical device of item 1937 wherein a portion of the electrical device is covered with a sleeve that contains the agent or the composition.

2161. The medical device of item 1937 wherein the electrical device is completely covered with a sleeve that contains the agent or the composition.

2162. The medical device of item 1937 wherein a portion of the electrical device is covered with a mesh that contains the agent or the composition.

2163. The medical device of item 1937 wherein the electrical device is completely covered with a mesh that contains the agent or the composition.

2164. The medical device of any one of items 1937-2163 wherein the bone growth stimulator comprises an electrode and a generator having a strain response piezoelectric material that responds to strain.

2165. A medical device, comprising a cardiac pacemaker (i.e., an electrical device) and an anti-scarring agent or a composition comprising an anti-scarring agent, wherein the agent inhibits scarring between the medical device and the host into which the medical device is implanted.

2166. The medical device of item 2165 wherein the agent is an adensosine A2A receptor antagonist.

2167. The medical device of item 2165 wherein the agent is an AKT inhibitor.

2168. The medical device of item 2165 wherein the agent is an alpha 2 integrin antagonist, wherein the alpha 2 integrin antagonist is Pharmaprojects No. 5754 (Merck KgaA).

2169. The medical device of item 2165 wherein the agent is an alpha 4 integrin antagonist.

2170. The medical device of item 2165 wherein the agent is an alpha 7 nicotinic receptor agonist.

2171. The medical device of item 2165 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

2172. The medical device of item 2165 wherein the agent is an apoptosis antagonist.

2173. The medical device of item 2165 wherein the agent is an apoptosis activator.

2174. The medical device of item 2165 wherein the agent is a beta 1 integrin antagonist.

2175. The medical device of item 2165 wherein the agent is a beta tubulin inhibitor.

2176. The medical device of item 2165 wherein the agent is a blocker of enzyme production in Hepatitis C.

2177. The medical device of item 2165 wherein the agent is a Bruton's tyrosine kinase inhibitor.

2178. The medical device of item 2165 wherein the agent is a calcineurin inhibitor.

2179. The medical device of item 2165 wherein the agent is a caspase 3 inhibitor.

2180. The medical device of item 2165 wherein the agent is a CC chemokine receptor antagonist.

2181. The medical device of item 2165 wherein the agent is a cell cycle inhibitor selected from the group consisting of SNS-595 (Sunesis), synthadotin, KRX-0403, and an analogue or derivative thereof.

2182. The medical device of item 2165 wherein the agent is a cathepsin B inhibitor.

2183. The medical device of item 2165 wherein the agent is a cathepsin K inhibitor, wherein the cathepsin K inhibitor is 462795 (GlaxoSmithKline), INPL-022-D6 (Amura Therapeutics), or an analogue or derivative thereof.

2184. The medical device of item 2165 wherein the agent is a cathepsin L inhibitor.

2185. The medical device of item 2165 wherein the agent is a CD40 antagonist.

2186. The medical device of item 2165 wherein the agent is a chemokine receptor agonist.

2187. The medical device of item 2165 wherein the agent is a chymase inhibitor.

2188. The medical device of item 2165 wherein the agent is a collagenase antagonist.

2189. The medical device of item 2165 wherein the agent is a CXCR antagonist.

2190. The medical device of item 2165 wherein the agent is a cyclin dependent kinase inhibitor selected from the group consisting of a CDK-1 inhibitor, a CDK-2 inhibitor, a CDK-4 inhibitor, a CDK-6 inhibitor, a CAKI inhibitor from GPC Biotech and Bristol-Myers Squibb, RGB-286199 (GPC Biotech), an anticancer agent from Astex Technology, a CAKI inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a SerfThr kinase inhibitor from Lilly (Eli Lilly), CVT-2584 (CAS No. 199986-75-9) (CV Therapeutics), and an analogue or derivative thereof.

2191. The medical device of item 2165 wherein the agent is a cyclooxygenase 1 inhibitor.

2192. The medical device of item 2165 wherein the agent is a DHFR inhibitor.

2193. The medical device of item 2165 wherein the agent is a dual integrin inhibitor.

2194. The medical device of item 2165 wherein the agent is an elastase inhibitor.

2195. The medical device of item 2165 wherein the agent is an elongation factor-1 alpha inhibitor.

2196. The medical device of item 2165 wherein the agent is an endothelial growth factor antagonist.

2197. The medical device of item 2165 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKiine), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

2198. The medical device of item 2165 wherein the agent is an endotoxin antagonist.

2199. The medical device of item 2165 wherein the agent is an epothilone and tubulin binder.

2200. The medical device of item 2165 wherein the agent is an estrogen receptor antagonist.

2201. The medical device of item 2165 wherein the agent is an FGF inhibitor.

2202. The medical device of item 2165 wherein the agent is a farnexyl transferase inhibitor.

2203. The medical device of item 2165 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

2204. The medical device of item 2165 wherein the agent is an FLT-3 kinase inhibitor.

2205. The medical device of item 2165 wherein the agent is an FGF receptor kinase inhibitor.

2206. The medical device of item 2165 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

2207. The medical device of item 2165 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

2208. The medical device of item 2165 wherein the agent is a histone deacetylase inhibitor.

2209. The medical device of item 2165 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

2210. The medical device of item 2165 wherein the agent is an ICAM inhibitor.

2211. The medical device of item 2165 wherein the agent is an IL, ICE and IRAK antagonist, wherein the antagonist is a CJ-14877, CP-424174 (Pfizer), NF-61 (Negma-Lerads), and an analogue or derivative thereof.

2212. The medical device of item 2165 wherein the agent is an IL-2 inhibitor.

2213. The medical device of item 2165 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

2214. The medical device of item 2165 wherein the agent is an IMPDH (inosine monophosphate).

2215. The medical device of item 2165 wherein the agent is an integrin antagonist.

2216. The medical device of item 2165 wherein the agent is an interleukin antagonist.

2217. The medical device of item 2165 wherein the agent is an inhibitor of type III receptor tyrosine kinase.

2218. The medical device of item 2165 wherein the agent is an irreversible inhibitor of enzyme methionine aminopeptidase type 2.

2219. The medical device of item 2165 wherein the agent is an isozyme selective delta protein kinase C inhibitor.

2220. The medical device of item 2165 wherein the agent a JAK3 enzyme inhibitor.

2221. The medical device of item 2165 wherein the agent is a JNK inhibitor.

2222. The medical device of item 2165 wherein the agent is a kinase inhibitor.

2223. The medical device of item 2165 wherein the agent is kinesin antagonist.

2224. The medical device of item 2165 wherein the agent is a kinesin antagonist.

2225. The medical device of item 2165 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

2226. The medical device of item 2165 wherein the agent is an MAP kinase inhibitor.

2227. The medical device of item 2165 wherein the agent is a matrix metalloproteinase inhibitor.

2228. The medical device of item 2165 wherein the agent is an MCP-CCR2 inhibitor.

2229. The medical device of item 2165 wherein the agent is an mTOR inhibitor.

2230. The medical device of item 2165 wherein the agent is an mTOR kinase inhibitor.

2231. The medical device of item 2165 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

2232. The medical device of item 2165 wherein the agent is an MIF inhibitor.

2233. The medical device of item 2165 wherein the agent is an MMP inhibitor.

2234. The medical device of item 2165 wherein the agent is a neurokinin (NK) antagonist selected from the group consisting of anthrotainin (CAS No. 14808440-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

2235. The medical device of item 2165 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

2236. The medical device of item 2165 wherein the agent is a nitric oxide agonist.

2237. The medical device of item 2165 wherein the agent is an ornithine decarboxylase inhibitor.

2238. The medical device of item 2165 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

2239. The medical device of item 2165 wherein the agent is a palmitoyl-protein thioesterase inhibitor.

2240. The medical device of item 2165 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

2241. The medical device of item 2165 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

2242. The medical device of item 2165 wherein the agent is a phosphatase inhibitor.

2243. The medical device of item 2165 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

2244. The medical device of item 2165 wherein the agent is a PKC inhibitor.

2245. The medical device of item 2165 wherein the agent is a platelet activating factor antagonist.

2246. The medical device of item 2165 wherein the agent is a platelet-derived growth factor receptor kinase inhibitor.

2247. The medical device of item 2165 wherein the agent is a prolyl hydroxylase inhibitor.

2248. The medical device of item 2165 wherein the agent is a polymorphonuclear neutrophil inhibitor.

2249. The medical device of item 2165 wherein the agent is a protein kinase B inhibitor.

2250. The medical device of item 2165 wherein the agent is a protein kinase C stimulant.

2251. The medical device of item 2165 wherein the agent is a purine nucleoside analogue.

2252. The medical device of item 2165 wherein the agent is a purinoreceptor P2X antagonist.

2253. The medical device of item 2165 wherein the agent is a Raf kinase inhibitor.

2254. The medical device of item 2165 wherein the agent is a reversible inhibitor of ErbB1 and ErbB2.

2255. The medical device of item 2165 wherein the agent is a ribonucleoside triphosphate reductase inhibitor.

2256. The medical device of item 2165 wherein the agent is an SDF-1 antagonist.

2257. The medical device of item 2165 wherein the agent is a sheddase inhibitor.

2258. The medical device of item 2165 wherein the agent is an SRC inhibitor.

2259. The medical device of item 2165 wherein the agent is a stromelysin inhibitor.

2260. The medical device of item 2165 wherein the agent is an Syk kinase inhibitor.

2261. The medical device of item 2165 wherein the agent is a telomerase inhibitor.

2262. The medical device of item 2165 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

2263. The medical device of item 2165 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

2264. The medical device of item 2165 wherein the agent is a Toll receptor inhibitor.

2265. The medical device of item 2165 wherein the agent is a tubulin antagonist.

2266. The medical device of item 2165 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

2267. The medical device of item 2165 wherein the agent is a VEGF inhibitor.

2268. The medical device of item 2165 wherein the agent is a vitamin D receptor agonist.

2269. The medical device of item 2165 wherein the agent is ZD-6474 (an angiogenesis inhibitor).

2270. The medical device of item 2165 wherein the agent is AP-23573 (an mTOR inhibitor).

2271. The medical device of item 2165 wherein the agent is synthadotin (a tubulin antagonist).

2272. The medical device of item 2165 wherein the agent is S-0885 (a collagenase inhibitor).

2273. The medical device of item 2165 wherein the agent is aplidine (an elongation factor-1 alpha inhibitor).

2274. The medical device of item 2165 wherein the agent is ixabepilone (an epithilone).

2275. The medical device of item 2165 wherein the agent is IDN-5390 (an angiogenesis inhibitor).

2276. The medical device of item 2165 wherein the agent is SB-2723005 (an angiogenesis inhibitor).

2277. The medical device of item 2165 wherein the agent is ABT-518 (an angiogenesis inhibitor).

2278. The medical device of item 2165 wherein the agent is combretastatin (an angiogenesis inhibitor).

2279. The medical device of item 2165 wherein the agent is anecortave acetate (an angiogenesis inhibitor).

2280. The medical device of item 2165 wherein the agent is SB-715992 (a kinesin antagonist).

2281. The medical device of item 2165 wherein the agent is temsirolimus (an mTOR inhibitor).

2282. The medical device of item 2165 wherein the agent is adalimumab (a TNFα antagonist).

2283. The medical device of item 2165, further comprising a coating, wherein the coating comprises the anti-scarring agent and a polymer.

2284. The medical device of item 2165, further comprising a coating, wherein the coating comprises the anti-scarring agent.

2285. The medical device of item 2165, further comprising a coating, wherein the coating is disposed on a surface of the electrical device.

2286. The medical device of item 2165, further comprising a coating, wherein the coating directly contacts the electrical device.

2287. The medical device of item 2165, further comprising a coating, wherein the coating indirectly contacts the electrical device.

2288. The medical device of item 2165, further comprising a coating, wherein the coating partially covers the electrical device.

2289. The medical device of item 2165, further comprising a coating, wherein the coating completely covers the electrical device.

2290. The medical device of item 2165, further comprising a coating, wherein the coating is a uniform coating.

2291. The medical device of item 2165, further comprising a coating, wherein the coating is a non-uniform coating.

2292. The medical device of item 2165, further comprising a coating, wherein the coating is a discontinuous coating.

2293. The medical device of item 2165, further comprising a coating, wherein the coating is a patterned coating.

2294. The medical device of item 2165, further comprising a coating, wherein the coating has a thickness of 100 μm or less.

2295. The medical device of item 2165, further comprising a coating, wherein the coating has a thickness of 10 μm or less.

2296. The medical device of item 2165, further comprising a coating, wherein the coating adheres to the surface of the electrical device upon deployment of the medical device.

2297. The medical device of item 2165, further comprising a coating, wherein the coating is stable at room temperature for a period of 1 year.

2298. The medical device of item 2165, further comprising a coating, wherein the anti-scarring agent is present in the coating in an amount ranging between about 0.0001% to about 1% by weight.

2299. The medical device of item 2165, further comprising a coating, wherein the anti-scarring agent is present in the coating in an amount ranging between about 1% to about 10% by weight.

2300. The medical device of item 2165, further comprising a coating, wherein the anti-scarring agent is present in the coating in an amount ranging between about 10% to about 25% by weight.

2301. The medical device of item 2165, further comprising a coating, wherein the anti-scarring agent is present in the coating in an amount ranging between about 25% to about 70% by weight.

2302. The medical device of item 2165, further comprising a coating, wherein the coating further comprises a polymer.

2303. The medical device of item 2165, further comprising a first coating having a first composition and the second coating having a second composition.

2304. The medical device of item 2165, further comprising a first coating having a first composition and the second coating having a second composition, wherein the first composition and the second composition are different.

2305. The medical device of item 2165, further comprising a polymer.

2306. The medical device of item 2165, further comprising a polymeric carrier.

2307. The medical device of item 2165, further comprising a polymeric carrier, wherein the polymeric carrier comprises a copolymer.

2308. The medical device of item 2165, further comprising a polymeric carrier, wherein the polymeric carrier comprises a block copolymer.

2309. The medical device of item 2165, further comprising a polymeric carrier, wherein the polymeric carrier comprises a random copolymer.

2310. The medical device of item 2165, further comprising a polymeric carrier, wherein the polymeric carrier comprises a biodegradable polymer.

2311. The medical device of item 2165, further comprising a polymeric carrier, wherein the polymeric carrier comprises a non-biodegradable polymer.

2312. The medical device of item 2165, further comprising a polymeric carrier, wherein the polymeric carrier comprises a hydrophilic polymer.

2313. The medical device of item 2165, further comprising a polymeric carrier, wherein the polymeric carrier comprises a hydrophobic polymer.

2314. The medical device of item 2165, further comprising a polymeric carrier, wherein the polymeric carrier comprises a polymer having hydrophilic domains.

2315. The medical device of item 2165, further comprising a polymeric carrier, wherein the polymeric carrier comprises a polymer having hydrophobic domains.

2316. The medical device of item 2165, further comprising a polymeric carrier, wherein the polymeric carrier comprises a non-conductive polymer.

2317. The medical device of item 2165, further comprising a polymeric carrier, wherein the polymeric carrier comprises an elastomer.

2318. The medical device of item 2165, further comprising a polymeric carrier, wherein the polymeric carrier comprises a hydrogel.

2319. The medical device of item 2165, further comprising a polymeric carrier, wherein the polymeric carrier comprises a silicone polymer.

2320. The medical device of item 2165, further comprising a polymeric carrier, wherein the polymeric carrier comprises a hydrocarbon polymer.

2321. The medical device of item 2165, further comprising a polymeric carrier, wherein the polymeric carrier comprises a styrene-derived polymer.

2322. The medical device of item 2165, further comprising a polymeric carrier, wherein the polymeric carrier comprises a butadiene polymer.

2323. The medical device of item 2165, further comprising a polymeric carrier, wherein the polymeric carrier comprises a macromer.

2324. The medical device of item 2165, further comprising a polymeric carrier, wherein the polymeric carrier comprises a poly(ethylene glycol) polymer.

2325. The medical device of item 2165, further comprising a polymeric carrier, wherein the polymeric carrier comprises an amorphous polymer.

2326. The medical device of item 2165, further comprising a lubricious coating.

2327. The medical device of item 2165 wherein the anti-scarring agent is located within pores or holes of the electrical device.

2328. The medical device of item 2165 wherein the anti-scarring agent is located within a channel, lumen, or divet of the electrical device.

2329. The medical device of item 2165, further comprising a second pharmaceutically active agent.

2330. The medical device of item 2165, further comprising an anti-inflammatory agent.

2331. The medical device of item 2165, further comprising an agent that inhibits infection.

2332. The medical device of item 2165, further comprising an agent that inhibits infection, wherein the agent is an anthracycline.

2333. The medical device of item 2165, further comprising an agent that inhibits infection, wherein the agent is doxorubicin.

2334. The medical device of item 2165, further comprising an agent that inhibits infection, wherein the agent is mitoxantrone.

2335. The medical device of item 2165, further comprising an agent that inhibits infection, wherein the agent is a fluoropyrimidine.

2336. The medical device of item 2165, further comprising an agent that inhibits infection, wherein the agent is 5-fluorouracil (5-FU).

2337. The medical device of item 2165, further comprising an agent that inhibits infection, wherein the agent is a folic acid antagonist.

2338. The medical device of item 2165, further comprising an agent that inhibits infection, wherein the agent is methotrexate.

2339. The medical device of item 2165, further comprising an agent that inhibits infection, wherein the agent is a podophylotoxin.

2340. The medical device of item 2165, further comprising an agent that inhibits infection, wherein the agent is etoposide.

2341. The medical device of item 2165, further comprising an agent that inhibits infection, wherein the agent is a camptothecin.

2342. The medical device of item 2165, further comprising an agent that inhibits infection, wherein the agent is a hydroxyurea.

2343. The medical device of item 2165, further comprising an agent that inhibits infection, wherein the agent is a platinum complex.

2344. The medical device of item 2165, further comprising an agent that inhibits infection, wherein the agent is cisplatin.

2345. The medical device of item 2165, further comprising an anti-thrombotic agent.

2346. The medical device of item 2165, further comprising a visualization agent.

2347. The medical device of item 2165, further comprising a visualization agent, wherein the visualization agent is a radiopaque material, wherein the radiopaque material comprises a metal, a halogenated compound, or a barium containing compound.

2348. The medical device of item 2165, further comprising a visualization agent, wherein the visualization agent is a radiopaque material, wherein the radiopaque material comprises barium, tantalum, or technetium.

2349. The medical device of item 2165, further comprising a visualization agent, wherein the visualization agent is a MRI responsive material.

2350. The medical device of item 2165, further comprising a visualization agent, wherein the visualization agent comprises a gadolinium chelate.

2351. The medical device of item 2165, further comprising a visualization agent, wherein the visualization agent comprises iron, magnesium, manganese, copper, or chromium.

2352. The medical device of item 2165, further comprising a visualization agent, wherein the visualization agent comprises an iron oxide compound.

2353. The medical device of item 2165, further comprising a visualization agent, wherein the visualization agent comprises a dye, pigment, or colorant.

2354. The medical device of item 2165, further comprising an echogenic material.

2355. The medical device of item 2165, further comprising an echogenic material, wherein the echogenic material is in the form of a coating.

2356. The medical device of item 2165 wherein the device is sterile.

2357. The medical device of item 2165 wherein the anti-scarring agent inhibits adhesion between the medical device and a host into which the medical device is implanted.

2358. The medical device of item 2165 wherein the medical device delivers the anti-scarring agent locally to tissue proximate to the medical device.

2359. The medical device of item 2165 wherein the anti-scarring agent is released into tissue in the vicinity of the medical device after deployment of the medical device.

2360. The medical device of item 2165 wherein the anti-scarring agent is released into tissue in the vicinity of the medical device after deployment of the medical device, wherein the tissue is connective tissue.

2361. The medical device of item 2165 wherein the anti-scarring agent is released into tissue in the vicinity of the medical device after deployment of the medical device, wherein the tissue is muscle tissue.

2362. The medical device of item 2165 wherein the anti-scarring agent is released into tissue in the vicinity of the medical device after deployment of the medical device, wherein the tissue is nerve tissue.

2363. The medical device of item 2165 wherein the anti-scarring agent is released into tissue in the vicinity of the medical device after deployment of the medical device, wherein the tissue is epithelium tissue.

2364. The medical device of item 2165 wherein the anti-scarring agent is released in effective concentrations from the medical device over a period ranging from the time of deployment of the medical device to about 1 year.

2365. The medical device of item 2165 wherein the anti-scarring agent is released in effective concentrations from the medical device over a period ranging from about 1 month to 6 months.

2366. The medical device of item 2165 wherein the anti-scarring agent is released in effective concentrations from the medical device over a period ranging from about 1-90 days.

2367. The medical device of item 2165 wherein the anti-scarring agent is released in effective concentrations from the medical device at a constant rate.

2368. The medical device of item 2165 wherein the anti-scarring agent is released in effective concentrations from the medical device at an increasing rate.

2369. The medical device of item 2165 wherein the anti-scarring agent is released in effective concentrations from the medical device at a decreasing rate.

2370. The medical device of item 2165 wherein the anti-scarring agent is released in effective concentrations from the composition comprising the anti-scarring agent by diffusion over a period ranging from the time of deployment of the medical device to about 90 days.

2371. The medical device of item 2165 wherein the anti-scarring agent is released in effective concentrations from the composition comprising the anti-scarring agent by erosion of the composition over a period ranging from the time of deployment of the medical device to about 90 days.

2372. The medical device of item 2165 wherein the device comprises about 0.01 μg to about 10 μg of the anti-scarring agent.

2373. The medical device of item 2165 wherein the device comprises about 10 μg to about 10 mg of the anti-scarring agent.

2374. The medical device of item 2165 wherein the device comprises about 10 mg to about 250 mg of the anti-scarring agent.

2375. The medical device of item 2165 wherein the device comprises about 250 mg to about 1000 mg of the anti-scarring agent.

2376. The medical device of item 2165 wherein the device comprises about 1000 mg to about 2500 mg of the anti-scarring agent.

2377. The medical device of item 2165 wherein a surface of the device comprises less than 0.01 μg of the anti-scarring agent per mm² of device surface to which the anti-scarring agent is applied.

2378. The medical device of item 2165 wherein a surface of the device comprises about 0.01 μg to about 1 μg of the anti-scarring agent per mm² of device surface to which the anti-scarring agent is applied.

2379. The medical device of item 2165 wherein a surface of the device comprises about 1 μg to about 10 μg of the anti-scarring agent per mm² of device surface to which the anti-scarring agent is applied.

2380. The medical device of item 2165 wherein a surface of the device comprises about 10 μg to about 250 μg of the anti-scarring agent per mm² of device surface to which the anti-scarring agent is applied.

2381. The medical device of item 2165 wherein a surface of the device comprises about 250 μg to about 1000 μg of the anti-scarring agent of anti-scarring agent per mm² of device surface to which the anti-scarring agent is applied.

2382. The medical device of item 2165 wherein a surface of the device comprises about 1000 μg to about 2500 μg of the anti-scarring agent per mm² of device surface to which the anti-scarring agent is applied.

2383. The medical device of item 2165 wherein the agent or the composition is affixed to the electrical device.

2384. The medical device of item 2165 wherein the agent or the composition is covalently attached to the electrical device.

2385. The medical device of item 2165 wherein the agent or the composition is non-covalently attached to the electrical device.

2386. The medical device of item 2165 further comprising a coating that absorbs the agent or the composition.

2387. The medical device of item 2165 wherein the electrical device is interweaved with a thread composed of, or coated with, the agent or the composition

2388. The medical device of item 2165 wherein a portion of the electrical device is covered with a sleeve that contains the agent or the composition.

2389. The medical device of item 2165 wherein the electrical device is completely covered with a sleeve that contains the agent or the composition.

2390. The medical device of item 2165 wherein a portion of the electrical device is covered with a mesh that contains the agent or the composition.

2391. The medical device of item 2165 wherein the electrical device is completely covered with a mesh that contains the agent or the composition.

2392. The medical device of any one of items 2165-2391 wherein the cardiac pacemaker is an adaptive rate pacemaker.

2393. The medical device of any one of items 2165-2391 wherein the cardiac pacemaker is a rate responsive pacemaker.

2394. A medical device, comprising an implantable cardioverter defibrillator (ICD) system (i.e., an electrical device) and an anti-scarring agent or a composition comprising an anti-scarring agent, wherein the agent inhibits scarring between the medical device and the host into which the medical device is implanted.

2395. The medical device of item 2394 wherein the agent is an adensosine A2A receptor antagonist.

2396. The medical device of item 2394 wherein the agent is an AKT inhibitor.

2397. The medical device of item 2394 wherein the agent is an alpha 2 integrin antagonist, wherein the alpha 2 integrin antagonist is Pharmaprojects No. 5754 (Merck KgaA).

2398. The medical device of item 2394 wherein the agent is an alpha 4 integrin antagonist.

2399. The medical device of item 2394 wherein the agent is an alpha 7 nicotinic receptor agonist.

2400. The medical device of item 2394 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

2401. The medical device of item 2394 wherein the agent is an apoptosis antagonist.

2402. The medical device of item 2394 wherein the agent is an apoptosis activator.

2403. The medical device of item 2394 wherein the agent is a beta 1 integrin antagonist.

2404. The medical device of item 2394 wherein the agent is a beta tubulin inhibitor.

2405. The medical device of item 2394 wherein the agent is a blocker of enzyme production in Hepatitis C.

2406. The medical device of item 2394 wherein the agent is a Bruton's tyrosine kinase inhibitor.

2407. The medical device of item 2394 wherein the agent is a calcineurin inhibitor.

2408. The medical device of item 2394 wherein the agent is a caspase 3 inhibitor.

2409. The medical device of item 2394 wherein the agent is a CC chemokine receptor antagonist.

2410. The medical device of item 2394 wherein the agent is a cell cycle inhibitor selected from the group consisting of SNS-595 (Sunesis), synthadotin, KRX-0403, and an analogue or derivative thereof.

2411. The medical device of item 2394 wherein the agent is a cathepsin B inhibitor.

2412. The medical device of item 2394 wherein the agent is a cathepsin K inhibitor, wherein the cathepsin K inhibitor is 462795 (GlaxoSmithKline), INPL-022-D6 (Amura Therapeutics), or an analogue or derivative thereof.

2413. The medical device of item 2394 wherein the agent is a cathepsin L inhibitor.

2414. The medical device of item 2394 wherein the agent is a CD40 antagonist.

2415. The medical device of item 2394 wherein the agent is a chemokine receptor agonist.

2416. The medical device of item 2394 wherein the agent is a chymase inhibitor.

2417. The medical device of item 2394 wherein the agent is a collagenase antagonist.

2418. The medical device of item 2394 wherein the agent is a CXCR antagonist.

2419. The medical device of item 2394 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Fhr kinase inhibitor from Lilly (Eli Lilly), CVT-2584 (CAS No. 199986-75-9) (CV Therapeutics), and an analogue or derivative thereof.

2420. The medical device of item 2394 wherein the agent is a cyclooxygenase 1 inhibitor.

2421. The medical device of item 2394 wherein the agent is a DHFR inhibitor.

2422. The medical device of item 2394 wherein the agent is a dual integrin inhibitor.

2423. The medical device of item 2394 wherein the agent is an elastase inhibitor.

2424. The medical device of item 2394 wherein the agent is an elongation factor-1 alpha inhibitor.

2425. The medical device of item 2394 wherein the agent is an endothelial growth factor antagonist.

2426. The medical device of item 2394 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

2427. The medical device of item 2394 wherein the agent is an endotoxin antagonist.

2428. The medical device of item 2394 wherein the agent is an epothilone and tubulin binder.

2429. The medical device of item 2394 wherein the agent is an estrogen receptor antagonist.

2430. The medical device of item 2394 wherein the agent is an FGF inhibitor.

2431. The medical device of item 2394 wherein the agent is a farnexyl transferase inhibitor.

2432. The medical device of item 2394 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

2433. The medical device of item 2394 wherein the agent is an FLT-3 kinase inhibitor.

2434. The medical device of item 2394 wherein the agent is an FGF receptor kinase inhibitor.

2435. The medical device of item 2394 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

2436. The medical device of item 2394 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

2437. The medical device of item 2394 wherein the agent is a histone deacetylase inhibitor.

2438. The medical device of item 2394 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

2439. The medical device of item 2394 wherein the agent is an ICAM inhibitor.

2440. The medical device of item 2394 wherein the agent is an IL, ICE and IRAK antagonist, wherein the antagonist is a CJ-14877, CP-424174 (Pfizer), NF-61 (Negma-Lerads), and an analogue or derivative thereof.

2441. The medical device of item 2394 wherein the agent is an IL-2 inhibitor.

2442. The medical device of item 2394 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

2443. The medical device of item 2394 wherein the agent is an IMPDH (inosine monophosphate).

2444. The medical device of item 2394 wherein the agent is an integrin antagonist.

2445. The medical device of item 2394 wherein the agent is an interleukin antagonist.

2446. The medical device of item 2394 wherein the agent is an inhibitor of type III receptor tyrosine kinase.

2447. The medical device of item 2394 wherein the agent is an irreversible inhibitor of enzyme methionine aminopeptidase type 2.

2448. The medical device of item 2394 wherein the agent is an isozyme selective delta protein kinase C inhibitor.

2449. The medical device of item 2394 wherein the agent a JAK3 enzyme inhibitor.

2450. The medical device of item 2394 wherein the agent is a JNK inhibitor

2451. The medical device of item 2394 wherein the agent is a kinase inhibitor.

2452. The medical device of item 2394 wherein the agent is kinesin antagonist.

2453. The medical device of item 2394 wherein the agent is a kinesin antagonist.

2454. The medical device of item 2394 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

2455. The medical device of item 2394 wherein the agent is an MAP kinase inhibitor.

2456. The medical device of item 2394 wherein the agent is a matrix metalloproteinase inhibitor

2457. The medical device of item 2394 wherein the agent is an MCP-CCR2 inhibitor.

2458. The medical device of item 2394 wherein the agent is an mTOR inhibitor.

2459. The medical device of item 2394 wherein the agent is an mTOR kinase inhibitor.

2460. The medical device of item 2394 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

2461. The medical device of item 2394 wherein the agent is an MIF inhibitor.

2462. The medical device of item 2394 wherein the agent is an MMP inhibitor.

2463. The medical device of item 2394 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

2464. The medical device of item 2394 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

2465. The medical device of item 2394 wherein the agent is a nitric oxide agonist.

2466. The medical device of item 2394 wherein the agent is an ornithine decarboxylase inhibitor.

2467. The medical device of item 2394 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

2468. The medical device of item 2394 wherein the agent is a palmitoyl-protein thioesterase inhibitor.

2469. The medical device of item 2394 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

2470. The medical device of item 2394 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-734 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

2471. The medical device of item 2394 wherein the agent is a phosphatase inhibitor.

2472. The medical device of item 2394 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

2473. The medical device of item 2394 wherein the agent is a PKC inhibitor.

2474. The medical device of item 2394 wherein the agent is a platelet activating factor antagonist.

2475. The medical device of item 2394 wherein the agent is a platelet-derived growth factor receptor kinase inhibitor.

2476. The medical device of item 2394 wherein the agent is a prolyl hydroxylase inhibitor.

2477. The medical device of item 2394 wherein the agent is a polymorphonuclear neutrophil inhibitor.

2478. The medical device of item 2394 wherein the agent is a protein kinase B inhibitor.

2479. The medical device of item 2394 wherein the agent is a protein kinase C stimulant.

2480. The medical device of item 2394 wherein the agent is a purine nucleoside analogue.

2481. The medical device of item 2394 wherein the agent is a purinoreceptor P2X antagonist.

2482. The medical device of item 2394 wherein the agent is a Raf kinase inhibitor.

2483. The medical device of item 2394 wherein the agent is a reversible inhibitor of ErbB1 and ErbB2.

2484. The medical device of item 2394 wherein the agent is a ribonucleoside triphosphate reductase inhibitor.

2485. The medical device of item 2394 wherein the agent is an SDF-1 antagonist.

2486. The medical device of item 2394 wherein the agent is a sheddase inhibitor.

2487. The medical device of item 2394 wherein the agent is an SRC inhibitor.

2488. The medical device of item 2394 wherein the agent is a stromelysin inhibitor.

2489. The medical device of item 2394 wherein the agent is an Syk kinase inhibitor.

2490. The medical device of item 2394 wherein the agent is a telomerase inhibitor.

2491. The medical device of item 2394 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

2492. The medical device of item 2394 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561 392 (Bristol-Myers Squibb), BN-006 (Bone), certolizumab pegol (CAS No. 428863-50-7 or CH-1 38 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifyline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

2493. The medical device of item 2394 wherein the agent is a Toll receptor inhibitor.

2494. The medical device of item 2394 wherein the agent is a tubulin antagonist.

2495. The medical device of item 2394 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

2496. The medical device of item 2394 wherein the agent is a VEGF inhibitor.

2497. The medical device of item 2394 wherein the agent is a vitamin D receptor agonist.

2498. The medical device of item 2394 wherein the agent is ZD-6474 (an angiogenesis inhibitor).

2499. The medical device of item 2394 wherein the agent is AP-23573 (an mTOR inhibitor).

2500. The medical device of item 2394 wherein the agent is synthadotin (a tubulin antagonist).

2501. The medical device of item 2394 wherein the agent is S-0885 (a collagenase inhibitor).

2502. The medical device of item 2394 wherein the agent is aplidine (an elongation factor-1 alpha inhibitor).

2503. The medical device of item 2394 wherein the agent is ixabepilone (an epithilone).

2504. The medical device of item 2394 wherein the agent is IDN-5390 (an angiogenesis inhibitor).

2505. The medical device of item 2394 wherein the agent is SB-2723005 (an angiogenesis inhibitor).

2506. The medical device of item 2394 wherein the agent is ABT-518 (an angiogenesis inhibitor).

2507. The medical device of item 2394 wherein the agent is combretastatin (an angiogenesis inhibitor).

2508. The medical device of item 2394 wherein the agent is anecortave acetate (an angiogenesis inhibitor).

2509. The medical device of item 2394 wherein the agent is SB-715992 (a kinesin antagonist).

2510. The medical device of item 2394 wherein the agent is temsirolimus (an mTOR inhibitor).

2511. The medical device of item 2394 wherein the agent is adalimumab (a TNFα antagonist).

2512. The medical device of item 2394, further comprising a coating, wherein the coating comprises the anti-scarring agent and a polymer.

2513. The medical device of item 2394, further comprising a coating, wherein the coating comprises the anti-scarring agent.

2514. The medical device of item 2394, further comprising a coating, wherein the coating is disposed on a surface of the electrical device.

2515. The medical device of item 2394, further comprising a coating, wherein the coating directly contacts the electrical device.

2516. The medical device of item 2394, further comprising a coating, wherein the coating indirectly contacts the electrical device.

2517. The medical device of item 2394, further comprising a coating, wherein the coating partially covers the electrical device.

2518. The medical device of item 2394, further comprising a coating, wherein the coating completely covers the electrical device.

2519. The medical device of item 2394, further comprising a coating, wherein the coating is a uniform coating.

2520. The medical device of item 2394, further comprising a coating, wherein the coating is a non-uniform coating.

2521. The medical device of item 2394, further comprising a coating, wherein the coating is a discontinuous coating.

2522. The medical device of item 2394, further comprising a coating, wherein the coating is a patterned coating.

2523. The medical device of item 2394, further comprising a coating, wherein the coating has a thickness of 100 μm or less.

2524. The medical device of item 2394, further comprising a coating, wherein the coating has a thickness of 10 μm or less.

2525. The medical device of item 2394, further comprising a coating, wherein the coating adheres to the surface of the electrical device upon deployment of the medical device.

2526. The medical device of item 2394, further comprising a coating, wherein the coating is stable at room temperature for a period of 1 year.

2527. The medical device of item 2394, further comprising a coating, wherein the anti-scarring agent is present in the coating in an amount ranging between about 0.0001% to about 1% by weight.

2528. The medical device of item 2394, further comprising a coating, wherein the anti-scarring agent is present in the coating in an amount ranging between about 1% to about 10% by weight.

2529. The medical device of item 2394, further comprising a coating, wherein the anti-scarring agent is present in the coating in an amount ranging between about 10% to about 25% by weight.

2530. The medical device of item 2394, further comprising a coating, wherein the anti-scarring agent is present in the coating in an amount ranging between about 25% to about 70% by weight.

2531. The medical device of item 2394, further comprising a coating, wherein the coating further comprises a polymer.

2532. The medical device of item 2394, further comprising a first coating having a first composition and the second coating having a second composition.

2533. The medical device of item 2394, further comprising a first coating having a first composition and the second coating having a second composition, wherein the first composition and the second composition are different.

2534. The medical device of item 2394, further comprising a polymer.

2535. The medical device of item 2394, further comprising a polymeric carrier.

2536. The medical device of item 2394, further comprising a polymeric carrier, wherein the polymeric carrier comprises a copolymer.

2537. The medical device of item 2394, further comprising a polymeric carrier, wherein the polymeric carrier comprises a block copolymer.

2538. The medical device of item 2394, further comprising a polymeric carrier, wherein the polymeric carrier comprises a random copolymer.

2539. The medical device of item 2394, further comprising a polymeric carrier, wherein the polymeric carrier comprises a biodegradable polymer.

2540. The medical device of item 2394, further comprising a polymeric carrier, wherein the polymeric carrier comprises a non-biodegradable polymer.

2541. The medical device of item 2394, further comprising a polymeric carrier, wherein the polymeric carrier comprises a hydrophilic polymer.

2542. The medical device of item 2394, further comprising a polymeric carrier, wherein the polymeric carrier comprises a hydrophobic polymer.

2543. The medical device of item 2394, further comprising a polymeric carrier, wherein the polymeric carrier comprises a polymer having hydrophilic domains.

2544. The medical device of item 2394, further comprising a polymeric carrier, wherein the polymeric carrier comprises a polymer having hydrophobic domains.

2545. The medical device of item 2394, further comprising a polymeric carrier, wherein the polymeric carrier comprises a non-conductive polymer.

2546. The medical device of item 2394, further comprising a polymeric carrier, wherein the polymeric carrier comprises an elastomer.

2547. The medical device of item 2394, further comprising a polymeric carrier, wherein the polymeric carrier comprises a hydrogel.

2548. The medical device of item 2394, further comprising a polymeric carrier, wherein the polymeric carrier comprises a silicone polymer.

2549. The medical device of item 2394, further comprising a polymeric carrier, wherein the polymeric carrier comprises a hydrocarbon polymer.

2550. The medical device of item 2394, further comprising a polymeric carrier, wherein the polymeric carrier comprises a styrene-derived polymer.

2551. The medical device of item 2394, further comprising a polymeric carrier, wherein the polymeric carrier comprises a butadiene polymer.

2552. The medical device of item 2394, further comprising a polymeric carrier, wherein the polymeric carrier comprises a macromer.

2553. The medical device of item 2394, further comprising a polymeric carrier, wherein the polymeric carrier comprises a poly(ethylene glycol) polymer.

2554. The medical device of item 2394, further comprising a polymeric carrier, wherein the polymeric carrier comprises an amorphous polymer.

2555. The medical device of item 2394, further comprising a lubricious coating.

2556. The medical device of item 2394 wherein the anti-scarring agent is located within pores or holes of the electrical device.

2557. The medical device of item 2394 wherein the anti-scarring agent is located within a channel, lumen, or divet of the electrical device.

2558. The medical device of item 2394, further comprising a second pharmaceutically active agent.

2559. The medical device of item 2394, further comprising an anti-inflammatory agent.

2560. The medical device of item 2394, further comprising an agent that inhibits infection.

2561. The medical device of item 2394, further comprising an agent that inhibits infection, wherein the agent is an anthracycline.

2562. The medical device of item 2394, further comprising an agent that inhibits infection, wherein the agent is doxorubicin.

2563. The medical device of item 2394, further comprising an agent that inhibits infection, wherein the agent is mitoxantrone.

2564. The medical device of item 2394, further comprising an agent that inhibits infection, wherein the agent is a fluoropyrimidine.

2565. The medical device of item 2394, further comprising an agent that inhibits infection, wherein the agent is 5-fluorouracil (5-FU).

2566. The medical device of item 2394, further comprising an agent that inhibits infection, wherein the agent is a folic acid antagonist.

2567. The medical device of item 2394, further comprising an agent that inhibits infection, wherein the agent is methotrexate.

2568. The medical device of item 2394, further comprising an agent that inhibits infection, wherein the agent is a podophylotoxin.

2569. The medical device of item 2394, further comprising an agent that inhibits infection, wherein the agent is etoposide.

2570. The medical device of item 2394, further comprising an agent that inhibits infection, wherein the agent is a camptothecin.

2571. The medical device of item 2394, further comprising an agent that inhibits infection, wherein the agent is a hydroxyurea.

2572. The medical device of item 2394, further comprising an agent that inhibits infection, wherein the agent is a platinum complex.

2573. The medical device of item 2394, further comprising an agent that inhibits infection, wherein the agent is cisplatin.

2574. The medical device of item 2394, further comprising an anti-thrombotic agent.

2575. The medical device of item 2394, further comprising a visualization agent.

2576. The medical device of item 2394, further comprising a visualization agent, wherein the visualization agent is a radiopaque material, wherein the radiopaque material comprises a metal, a halogenated compound, or a barium containing compound.

2577. The medical device of item 2394, further comprising a visualization agent, wherein the visualization agent is a radiopaque material, wherein the radiopaque material comprises barium, tantalum, or technetium.

2578. The medical device of item 2394, further comprising a visualization agent, wherein the visualization agent is a MRI responsive material.

2579. The medical device of item 2394, further comprising a visualization agent, wherein the visualization agent comprises a gadolinium chelate.

2580. The medical device of item 2394, further comprising a visualization agent, wherein the visualization agent comprises iron, magnesium, manganese, copper, or chromium.

2581. The medical device of item 2394, further comprising a visualization agent, wherein the visualization agent comprises an iron oxide compound.

2582. The medical device of item 2394, further comprising a visualization agent, wherein the visualization agent comprises a dye, pigment, or colorant

2583. The medical device of item 2394, further comprising an echogenic material.

2584. The medical device of item 2394, further comprising an echogenic material, wherein the echogenic material is in the form of a coating.

2585. The medical device of item 2394 wherein the device is sterile.

2586. The medical device of item 2394 wherein the anti-scarring agent inhibits adhesion between the medical device and a host into which the medical device is implanted.

2587. The medical device of item 2394 wherein the medical device delivers the anti-scarring agent locally to tissue proximate to the medical device.

2588. The medical device of item 2394 wherein the anti-scarring agent is released into tissue in the vicinity of the medical device after deployment of the medical device.

2589. The medical device of item 2394 wherein the anti-scarring agent is released into tissue in the vicinity of the medical device after deployment of the medical device, wherein the tissue is connective tissue.

2590. The medical device of item 2394 wherein the anti-scarring agent is released into tissue in the vicinity of the medical device after deployment of the medical device, wherein the tissue is muscle tissue.

2591. The medical device of item 2394 wherein the anti-scarring agent is released into tissue in the vicinity of the medical device after deployment of the medical device, wherein the tissue is nerve tissue.

2592. The medical device of item 2394 wherein the anti-scarring agent is released into tissue in the vicinity of the medical device after deployment of the medical device, wherein the tissue is epithelium tissue.

2593. The medical device of item 2394 wherein the anti-scarring agent is released in effective concentrations from the medical device over a period ranging from the time of deployment of the medical device to about 1 year.

2594. The medical device of item 2394 wherein the anti-scarring agent is released in effective concentrations from the medical device over a period ranging from about 1 month to 6 months.

2595. The medical device of item 2394 wherein the anti-scarring agent is released in effective concentrations from the medical device over a period ranging from about 1-90 days.

2596. The medical device of item 2394 wherein the anti-scarring agent is released in effective concentrations from the medical device at a constant rate.

2597. The medical device of item 2394 wherein the anti-scarring agent is released in effective concentrations from the medical device at an increasing rate.

2598. The medical device of item 2394 wherein the anti-scarring agent is released in effective concentrations from the medical device at a decreasing rate

2599. The medical device of item 2394 wherein the anti-scarring agent is released in effective concentrations from the composition comprising the anti-scarring agent by diffusion over a period ranging from the time of deployment of the medical device to about 90 days.

2600. The medical device of item 2394 wherein the anti-scarring agent is released in effective concentrations from the composition comprising the anti-scarring agent by erosion of the composition over a period ranging from the time of deployment of the medical device to about 90 days.

2601. The medical device of item 2394 wherein the device comprises about 0.01 μg to about 10 μg of the anti-scarring agent.

2602. The medical device of item 2394 wherein the device comprises about 10 μg to about 10 mg of the anti-scarring agent.

2603. The medical device of item 2394 wherein the device comprises about 10 mg to about 250 mg of the anti-scarring agent.

2604. The medical device of item 2394 wherein the device comprises about 250 mg to about 1000 mg of the anti-scarring agent.

2605. The medical device of item 2394 wherein the device comprises about 1000 mg to about 2500 mg of the anti-scarring agent.

2606. The medical device of item 2394 wherein a surface of the device comprises less than 0.01 μg of the anti-scarring agent per mm² of device surface to which the anti-scarring agent is applied.

2607. The medical device of item 2394 wherein a surface of the device comprises about 0.01 μg to about 1 μg of the anti-scarring agent per mm² of device surface to which the anti-scarring agent is applied.

2608. The medical device of item 2394 wherein a surface of the device comprises about 1 μg to about 10 μg of the anti-scarring agent per mm² of device surface to which the anti-scarring agent is applied.

2609. The medical device of item 2394 wherein a surface of the device comprises about 10 μg to about 250 μg of the anti-scarring agent per mm² of device surface to which the anti-scarring agent is applied.

2610. The medical device of item 2394 wherein a surface of the device comprises about 250 μg to about 1000 μg of the anti-scarring agent of anti-scarring agent per mm² of device surface to which the anti-scarring agent is applied.

2611. The medical device of item 2394 wherein a surface of the device comprises about 1000 μg to about 2500 μg of the anti-scarring agent per mm² of device surface to which the anti-scarring agent is applied.

2612. The medical device of item 2394 wherein the agent or the composition is affixed to the electrical device.

2613. The medical device of item 2394 wherein the agent or the composition is covalently attached to the electrical device.

2614. The medical device of item 2394 wherein the agent or the composition is non-covalently attached to the electrical device.

2615. The medical device of item 2394 further comprising a coating that absorbs the agent or the composition.

2616. The medical device of item 2394 wherein the electrical device is interweaved with a thread composed of, or coated with, the agent or the composition.

2617. The medical device of item 2394 wherein a portion of the electrical device is covered with a sleeve that contains the agent or the composition.

2618. The medical device of item 2394 wherein the electrical device is completely covered with a sleeve that contains the agent or the composition.

2619. The medical device of item 2394 wherein a portion of the electrical device is covered with a mesh that contains the agent or the composition.

2620. The medical device of item 2394 wherein the electrical device is completely covered with a mesh that contains the agent or the composition.

2621. The medical device of any one of items 2394-2620 wherein the implantable cardioverter defibrillator is adapted for treating tachyarraythmias.

2622. The medical device of any one of items 2394-2620 wherein the implantable cardioverter defibrillator is adapted for ventricular tachycardia.

2623. The medical device of any one of items 2394-2620 wherein the implantable cardioverter defibrillator is adapted for treating ventricular fibrillation.

2624. The medical device of any one of items 2394-2620 wherein the implantable cardioverter defibrillator is adapted for treating atrial tachycardia.

2625. The medical device of any one of items 2394-2620 wherein the implantable cardioverter defibrillator is adapted for treating atrial fibrillation.

2626. The medical device of any one of items 2394-2620 wherein the implantable cardioverter defibrillator is adapted for treating arrhythmias.

2627. A medical device, comprising a vagus nerve stimulator for treating arrhythemia (i.e., an electrical device) and an anti-scarring agent or a composition comprising an anti-scarring agent, wherein the agent inhibits scarring between the medical device and the host into which the medical device is implanted.

2628. The medical device of item 2627 wherein the agent is an adensosine A2A receptor antagonist.

2629. The medical device of item 2627 wherein the agent is an AKT inhibitor.

2630. The medical device of item 2627 wherein the agent is an alpha 2 integrin antagonist, wherein the alpha 2 integrin antagonist is Pharmaprojects No. 5754 (Merck KgaA).

2631. The medical device of item 2627 wherein the agent is an alpha 4 integrin antagonist.

2632. The medical device of item 2627 wherein the agent is an alpha 7 nicotinic receptor agonist.

2633. The medical device of item 2627 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

2634. The medical device of item 2627 wherein the agent is an apoptosis antagonist.

2635. The medical device of item 2627 wherein the agent is an apoptosis activator.

2636. The medical device of item 2627 wherein the agent is a beta 1 integrin antagonist.

2637. The medical device of item 2627 wherein the agent is a beta tubulin inhibitor.

2638. The medical device of item 2627 wherein the agent is a blocker of enzyme production in Hepatitis C.

2639. The medical device of item 2627 wherein the agent is a Bruton's tyrosine kinase inhibitor.

2640. The medical device of item 2627 wherein the agent is a calcineurin inhibitor.

2641. The medical device of item 2627 wherein the agent is a caspase 3 inhibitor.

2642. The medical device of item 2627 wherein the agent is a CC chemokine receptor antagonist.

2643. The medical device of item 2627 wherein the agent is a cell cycle inhibitor selected from the group consisting of SNS-595 (Sunesis), synthadotin, KRX-0403, and an analogue or derivative thereof.

2644. The medical device of item 2627 wherein the agent is a cathepsin B inhibitor.

2645. The medical device of item 2627 wherein the agent is a cathepsin K inhibitor, wherein the cathepsin K inhibitor is 462795 (GlaxoSmithKline), INPL-022-D6 (Amura Therapeutics), or an analogue or derivative thereof.

2646. The medical device of item 2627 wherein the agent is a cathepsin L inhibitor.

2647. The medical device of item 2627 wherein the agent is a CD40 antagonist.

2648. The medical device of item 2627 wherein the agent is a chemokine receptor agonist.

2649. The medical device of item 2627 wherein the agent is a chymase inhibitor.

2650. The medical device of item 2627 wherein the agent is a collagenase antagonist.

2651. The medical device of item 2627 wherein the agent is a CXCR antagonist.

2652. The medical device of item 2627 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-2861 99 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

2653. The medical device of item 2627 wherein the agent is a cyclooxygenase 1 inhibitor.

2654. The medical device of item 2627 wherein the agent is a DHFR inhibitor.

2655. The medical device of item 2627 wherein the agent is a dual integrin inhibitor.

2656. The medical device of item 2627 wherein the agent is an elastase inhibitor.

2657. The medical device of item 2627 wherein the agent is an elongation factor-1 alpha inhibitor.

2658. The medical device of item 2627 wherein the agent is an endothelial growth factor antagonist.

2659. The medical device of item 2627 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

2660. The medical device of item 2627 wherein the agent is an endotoxin antagonist.

2661. The medical device of item 2627 wherein the agent is an epothilone and tubulin binder.

2662. The medical device of item 2627 wherein the agent is an estrogen receptor antagonist.

2663. The medical device of item 2627 wherein the agent is an FGF inhibitor.

2664. The medical device of item 2627 wherein the agent is a farnexyl transferase inhibitor.

2665. The medical device of item 2627 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

2666. The medical device of item 2627 wherein the agent is an FLT-3 kinase inhibitor.

2667. The medical device of item 2627 wherein the agent is an FGF receptor kinase inhibitor.

2668. The medical device of item 2627 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

2669. The medical device of item 2627 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

2670. The medical device of item 2627 wherein the agent is a histone deacetylase inhibitor.

2671. The medical device of item 2627 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

2672. The medical device of item 2627 wherein the agent is an ICAM inhibitor.

2673. The medical device of item 2627 wherein the agent is an IL, ICE and IRAK antagonist, wherein the antagonist is a CJ-14877, CP-424174 (Pfizer), NF-61 (Negma-Lerads), and an analogue or derivative thereof.

2674. The medical device of item 2627 wherein the agent is an IL-2 inhibitor.

2675. The medical device of item 2627 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

2676. The medical device of item 2627 wherein the agent is an IMPDH (inosine monophosphate).

2677. The medical device of item 2627 wherein the agent is an integrin antagonist.

2678. The medical device of item 2627 wherein the agent is an interleukin antagonist.

2679. The medical device of item 2627 wherein the agent is an inhibitor of type III receptor tyrosine kinase.

2680. The medical device of item 2627 wherein the agent is an irreversible inhibitor of enzyme methionine aminopeptidase type 2.

2681. The medical device of item 2627 wherein the agent is an isozyme selective delta protein kinase C inhibitor.

2682. The medical device of item 2627 wherein the agent a JAK3 enzyme inhibitor.

2683. The medical device of item 2627 wherein the agent is a INK inhibitor.

2684. The medical device of item 2627 wherein the agent is a kinase inhibitor.

2685. The medical device of item 2627 wherein the agent is kinesin antagonist.

2686. The medical device of item 2627 wherein the agent is a kinesin antagonist.

2687. The medical device of item 2627 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

2688. The medical device of item 2627 wherein the agent is an MAP kinase inhibitor.

2689. The medical device of item 2627 wherein the agent is a matrix metalloproteinase inhibitor.

2690. The medical device of item 2627 wherein the agent is an MCP-CCR2 inhibitor.

2691. The medical device of item 2627 wherein the agent is an mTOR inhibitor.

2692. The medical device of item 2627 wherein the agent is an mTOR kinase inhibitor.

2693. The medical device of item 2627 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

2694. The medical device of item 2627 wherein the agent is an MIF inhibitor.

2695. The medical device of item 2627 wherein the agent is an MMP inhibitor.

2696. The medical device of item 2627 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

2697. The medical device of item 2627 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

2698. The medical device of item 2627 wherein the agent is a nitric oxide agonist.

2699. The medical device of item 2627 wherein the agent is an ornithine decarboxylase inhibitor.

2700. The medical device of item 2627 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

2701. The medical device of item 2627 wherein the agent is a palmitoyl-protein thioesterase inhibitor.

2702. The medical device of item 2627 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof

2703. The medical device of item 2627 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

2704. The medical device of item 2627 wherein the agent is a phosphatase inhibitor.

2705. The medical device of item 2627 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

2706. The medical device of item 2627 wherein the agent is a PKC inhibitor.

2707. The medical device of item 2627 wherein the agent is a platelet activating factor antagonist.

2708. The medical device of item 2627 wherein the agent is a platelet-derived growth factor receptor kinase inhibitor.

2709. The medical device of item 2627 wherein the agent is a prolyl hydroxylase inhibitor.

2710. The medical device of item 2627 wherein the agent is a polymorphonuclear neutrophil inhibitor.

2711. The medical device of item 2627 wherein the agent is a protein kinase B inhibitor.

2712. The medical device of item 2627 wherein the agent is a protein kinase C stimulant.

2713. The medical device of item 2627 wherein the agent is a purine nucleoside analogue.

2714. The medical device of item 2627 wherein the agent is a purinoreceptor P2X antagonist.

2715. The medical device of item 2627 wherein the agent is a Raf kinase inhibitor.

2716. The medical device of item 2627 wherein the agent is a reversible inhibitor of ErbB1 and ErbB2.

2717. The medical device of item 2627 wherein the agent is a ribonucleoside triphosphate reductase inhibitor.

2718. The medical device of item 2627 wherein the agent is an SDF-1 antagonist.

2719. The medical device of item 2627 wherein the agent is a sheddase inhibitor.

2720. The medical device of item 2627 wherein the agent is an SRC inhibitor.

2721. The medical device of item 2627 wherein the agent is a stromelysin inhibitor.

2722. The medical device of item 2627 wherein the agent is an Syk kinase inhibitor.

2723. The medical device of item 2627 wherein the agent is a telomerase inhibitor.

2724. The medical device of item 2627 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

2725. The medical device of item 2627 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

2726. The medical device of item 2627 wherein the agent is a Toll receptor inhibitor.

2727. The medical device of item 2627 wherein the agent is a tubulin antagonist.

2728. The medical device of item 2627 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-133022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

2729. The medical device of item 2627 wherein the agent is a VEGF inhibitor.

2730. The medical device of item 2627 wherein the agent is a vitamin D receptor agonist.

2731. The medical device of item 2627 wherein the agent is ZD-6474 (an angiogenesis inhibitor).

2732. The medical device of item 2627 wherein the agent is AP-23573 (an mTOR inhibitor).

2733. The medical device of item 2627 wherein the agent is synthadotin (a tubulin antagonist).

2734. The medical device of item 2627 wherein the agent is S-0885 (a collagenase inhibitor).

2735. The medical device of item 2627 wherein the agent is aplidine (an elongation factor-1 alpha inhibitor).

2736. The medical device of item 2627 wherein the agent is ixabepilone (an epithilone).

2737. The medical device of item 2627 wherein the agent is IDN-5390 (an angiogenesis inhibitor).

2738. The medical device of item 2627 wherein the agent is SB-2723005 (an angiogenesis inhibitor).

2739. The medical device of item 2627 wherein the agent is ABT-518 (an angiogenesis inhibitor).

2740. The medical device of item 2627 wherein the agent is combretastatin (an angiogenesis inhibitor).

2741. The medical device of item 2627 wherein the agent is anecortave acetate (an angiogenesis inhibitor).

2742. The medical device of item 2627 wherein the agent is SB-715992 (a kinesin antagonist).

2743. The medical device of item 2627 wherein the agent is temsirolimus (an mTOR inhibitor).

2744. The medical device of item 2627 wherein the agent is adalimumab (a TNFα antagonist).

2745. The medical device of item 2627, further comprising a coating, wherein the coating comprises the anti-scarring agent and a polymer.

2746. The medical device of item 2627, further comprising a coating, wherein the coating comprises the anti-scarring agent.

2747. The medical device of item 2627, further comprising a coating, wherein the coating is disposed on a surface of the electrical device.

2748. The medical device of item 2627, further comprising a coating, wherein the coating directly contacts the electrical device.

2749. The medical device of item 2627, further comprising a coating, wherein the coating indirectly contacts the electrical device.

2750. The medical device of item 2627, further comprising a coating, wherein the coating partially covers the electrical device.

2751. The medical device of item 2627, further comprising a coating, wherein the coating completely covers the electrical device.

2752. The medical device of item 2627, further comprising a coating, wherein the coating is a uniform coating.

2753. The medical device of item 2627, further comprising a coating, wherein the coating is a non-uniform coating.

2754. The medical device of item 2627, further comprising a coating, wherein the coating is a discontinuous coating.

2755. The medical device of item 2627, further comprising a coating, wherein the coating is a patterned coating.

2756. The medical device of item 2627, further comprising a coating, wherein the coating has a thickness of 100 μm or less.

2757. The medical device of item 2627, further comprising a coating, wherein the coating has a thickness of 10 pm or less.

2758. The medical device of item 2627, further comprising a coating, wherein the coating adheres to the surface of the electrical device upon deployment of the medical device.

2759. The medical device of item 2627, further comprising a coating, wherein the coating is stable at room temperature for a period of 1 year.

2760. The medical device of item 2627, further comprising a coating, wherein the anti-scarring agent is present in the coating in an amount ranging between about 0.0001% to about 1% by weight.

2761. The medical device of item 2627, further comprising a coating, wherein the anti-scarring agent is present in the coating in an amount ranging between about 1% to about 10% by weight.

2762. The medical device of item 2627, further comprising a coating, wherein the anti-scarring agent is present in the coating in an amount ranging between about 10% to about 25% by weight.

2763. The medical device of item 2627, further comprising a coating, wherein the anti-scarring agent is present in the coating in an amount ranging between about 25% to about 70% by weight.

2764. The medical device of item 2627, further comprising a coating, wherein the coating further comprises a polymer.

2765. The medical device of item 2627, further comprising a first coating having a first composition and the second coating having a second composition.

2766. The medical device of item 2627, further comprising a first coating having a first composition and the second coating having a second composition, wherein the first composition and the second composition are different.

2767. The medical device of item 2627, further comprising a polymer.

2768. The medical device of item 2627, further comprising a polymeric carrier.

2769. The medical device of item 2627, further comprising a polymeric carrier, wherein the polymeric carrier comprises a copolymer.

2770. The medical device of item 2627, further comprising a polymeric carrier, wherein the polymeric carrier comprises a block copolymer.

2771. The medical device of item 2627, further comprising a polymeric carrier, wherein the polymeric carrier comprises a random copolymer.

2772. The medical device of item 2627, further comprising a polymeric carrier, wherein the polymeric carrier comprises a biodegradable polymer.

2773. The medical device of item 2627, further comprising a polymeric carrier, wherein the polymeric carrier comprises a non-biodegradable polymer.

2774. The medical device of item 2627, further comprising a polymeric carrier, wherein the polymeric carrier comprises a hydrophilic polymer.

2775. The medical device of item 2627, further comprising a polymeric carrier, wherein the polymeric carrier comprises a hydrophobic polymer.

2776. The medical device of item 2627, further comprising a polymeric carrier, wherein the polymeric carrier comprises a polymer having hydrophilic domains.

2777. The medical device of item 2627, further comprising a polymeric carrier, wherein the polymeric carrier comprises a polymer having hydrophobic domains.

2778. The medical device of item 2627, further comprising a polymeric carrier, wherein the polymeric carrier comprises a non-conductive polymer.

2779. The medical device of item 2627, further comprising a polymeric carrier, wherein the polymeric carrier comprises an elastomer.

2780. The medical device of item 2627, further comprising a polymeric carrier, wherein the polymeric carrier comprises a hydrogel.

2781. The medical device of item 2627, further comprising a polymeric carrier, wherein the polymeric carrier comprises a silicone polymer.

2782. The medical device of item 2627, further comprising a polymeric carrier, wherein the polymeric carrier comprises a hydrocarbon polymer.

2783. The medical device of item 2627, further comprising a polymeric carrier, wherein the polymeric carrier comprises a styrene-derived polymer.

2784. The medical device of item 2627, further comprising a polymeric carrier, wherein the polymeric carrier comprises a butadiene polymer.

2785. The medical device of item 2627, further comprising a polymeric carrier, wherein the polymeric carrier comprises a macromer.

2786. The medical device of item 2627, further comprising a polymeric carrier, wherein the polymeric carrier comprises a poly(ethylene glycol) polymer.

2787. The medical device of item 2627, further comprising a polymeric carrier, wherein the polymeric carrier comprises an amorphous polymer.

2788. The medical device of item 2627, further comprising a lubricious coating.

2789. The medical device of item 2627 wherein the anti-scarring agent is located within pores or holes of the electrical device.

2790. The medical device of item 2627 wherein the anti-scarring agent is located within a channel, lumen, or divet of the electrical device.

2791. The medical device of item 2627, further comprising a second pharmaceutically active agent.

2792. The medical device of item 2627, further comprising an anti-inflammatory agent.

2793. The medical device of item 2627, further comprising an agent that inhibits infection.

2794. The medical device of item 2627, further comprising an agent that inhibits infection, wherein the agent is an anthracycline.

2795. The medical device of item 2627, further comprising an agent that inhibits infection, wherein the agent is doxorubicin.

2796. The medical device of item 2627, further comprising an agent that inhibits infection, wherein the agent is mitoxantrone.

2797. The medical device of item 2627, further comprising an agent that inhibits infection, wherein the agent is a fluoropyrimidine.

2798. The medical device of item 2627, further comprising an agent that inhibits infection, wherein the agent is 5-fluorouracil (5-FU).

2799. The medical device of item 2627, further comprising an agent that inhibits infection, wherein the agent is a folic acid antagonist.

2800. The medical device of item 2627, further comprising an agent that inhibits infection, wherein the agent is methotrexate.

2801. The medical device of item 2627, further comprising an agent that inhibits infection, wherein the agent is a podophylotoxin.

2802. The medical device of item 2627, further comprising an agent that inhibits infection, wherein the agent is etoposide.

2803. The medical device of item 2627, further comprising an agent that inhibits infection, wherein the agent is a camptothecin.

2804. The medical device of item 2627, further comprising an agent that inhibits infection, wherein the agent is a hydroxyurea.

2805. The medical device of item 2627, further comprising an agent that inhibits infection, wherein the agent is a platinum complex.

2806. The medical device of item 2627, further comprising an agent that inhibits infection, wherein the agent is cisplatin.

2807. The medical device of item 2627, further comprising an anti-thrombotic agent.

2808. The medical device of item 2627, further comprising a visualization agent.

2809. The medical device of item 2627, further comprising a visualization agent, wherein the visualization agent is a radiopaque material, wherein the radiopaque material comprises a metal, a halogenated compound, or a barium containing compound.

2810. The medical device of item 2627, further comprising a visualization agent, wherein the visualization agent is a radiopaque material, wherein the radiopaque material comprises barium, tantalum, or technetium.

2811. The medical device of item 2627, further comprising a visualization agent, wherein the visualization agent is a MRI responsive material.

2812. The medical device of item 2627, further comprising a visualization agent, wherein the visualization agent comprises a gadolinium chelate.

2813. The medical device of item 2627, further comprising a visualization agent, wherein the visualization agent comprises iron, magnesium, manganese, copper, or chromium.

2814. The medical device of item 2627, further comprising a visualization agent, wherein the visualization agent comprises an iron oxide compound.

2815. The medical device of item 2627, further comprising a visualization agent, wherein the visualization agent comprises a dye, pigment, or colorant.

2816. The medical device of item 2627, further comprising an echogenic material.

2817. The medical device of item 2627, further comprising an echogenic material, wherein the echogenic material is in the form of a coating.

2818. The medical device of item 2627 wherein the device is sterile.

2819. The medical device of item 2627 wherein the anti-scarring agent inhibits adhesion between the medical device and a host into which the medical device is implanted.

2820. The medical device of item 2627 wherein the medical device delivers the anti-scarring agent locally to tissue proximate to the medical device.

2821. The medical device of item 2627 wherein the anti-scarring agent is released into tissue in the vicinity of the medical device after deployment of the medical device.

2822. The medical device of item 2627 wherein the anti-scarring agent is released into tissue in the vicinity of the medical device after deployment of the medical device, wherein the tissue is connective tissue.

2823. The medical device of item 2627 wherein the anti-scarring agent is released into tissue in the vicinity of the medical device after deployment of the medical device, wherein the tissue is muscle tissue.

2824. The medical device of item 2627 wherein the anti-scarring agent is released into tissue in the vicinity of the medical device after deployment of the medical device, wherein the tissue is nerve tissue.

2825. The medical device of item 2627 wherein the anti-scarring agent is released into tissue in the vicinity of the medical device after deployment of the medical device, wherein the tissue is epithelium tissue.

2826. The medical device of item 2627 wherein the anti-scarring agent is released in effective concentrations from the medical device over a period ranging from the time of deployment of the medical device to about 1 year.

2827. The medical device of item 2627 wherein the anti-scarring agent is released in effective concentrations from the medical device over a period ranging from about 1 month to 6 months.

2828. The medical device of item 2627 wherein the anti-scarring agent is released in effective concentrations from the medical device over a period ranging from about 1-90 days.

2829. The medical device of item 2627 wherein the anti-scarring agent is released in effective concentrations from the medical device at a constant rate.

2830. The medical device of item 2627 wherein the anti-scarring agent is released in effective concentrations from the medical device at an increasing rate.

2831. The medical device of item 2627 wherein the anti-scarring agent is released in effective concentrations from the medical device at a decreasing rate.

2832. The medical device of item 2627 wherein the anti-scarring agent is released in effective concentrations from the composition comprising the anti-scarring agent by diffusion over a period ranging from the time of deployment of the medical device to about 90 days.

2833. The medical device of item 2627 wherein the anti-scarring agent is released in effective concentrations from the composition comprising the anti-scarring agent by erosion of the composition over a period ranging from the time of deployment of the medical device to about 90 days.

2834. The medical device of item 2627 wherein the device comprises about 0.01 μg to about 10 μg of the anti-scarring agent.

2835. The medical device of item 2627 wherein the device comprises about 10 μg to about 10 mg of the anti-scarring agent.

2836. The medical device of item 2627 wherein the device comprises about 10 mg to about 250 mg of the anti-scarring agent.

2837. The medical device of item 2627 wherein the device comprises about 250 mg to about 1000 mg of the anti-scarring agent.

2838. The medical device of item 2627 wherein the device comprises about 1000 mg to about 2500 mg of the anti-scarring agent.

2839. The medical device of item 2627 wherein a surface of the device comprises less than 0.01 μg of the anti-scarring agent per mm² of device surface to which the anti-scarring agent is applied.

2840. The medical device of item 2627 wherein a surface of the device comprises about 0.01 μg to about 1 μg of the anti-scarring agent per mm² of device surface to which the anti-scarring agent is applied.

2841. The medical device of item 2627 wherein a surface of the device comprises about 1 μg to about 10 μg of the anti-scarring agent per mm² of device surface to which the anti-scarring agent is applied

2842. The medical device of item 2627 wherein a surface of the device comprises about 10 μg to about 250 μg of the anti-scarring agent per mm² of device surface to which the anti-scarring agent is applied.

2843. The medical device of item 2627 wherein a surface of the device comprises about 250 μg to about 1000 μg of the anti-scarring agent of anti-scarring agent per mm² of device surface to which the anti-scarring agent is applied.

2844. The medical device of item 2627 wherein a surface of the device comprises about 1000 μg to about 2500 μg of the anti-scarring agent per mm² of device surface to which the anti-scarring agent is applied.

2845. The medical device of item 2627 wherein the agent or the composition is affixed to the electrical device.

2846. The medical device of item 2627 wherein the agent or the composition is covalently attached to the electrical device.

2847. The medical device of item 2627 wherein the agent or the composition is non-covalently attached to the electrical device.

2848. The medical device of item 2627 further comprising a coating that absorbs the agent or the composition.

2849. The medical device of item 2627 wherein the electrical device is interweaved with a thread composed of, or coated with, the agent or the composition.

2850. The medical device of item 2627 wherein a portion of the electrical device is covered with a sleeve that contains the agent or the composition.

2851. The medical device of item 2627 wherein the electrical device is completely covered with a sleeve that contains the agent or the composition.

2852. The medical device of item 2627 wherein a portion of the electrical device is covered with a mesh that contains the agent or the composition.

2853. The medical device of item 2627 wherein the electrical device is completely covered with a mesh that contains the agent or the composition.

2854. The medical device of any one of items 2627-2853 wherein the vagus nerve stimulator is adapted for treating supraventricular arrhythmias.

2855. The medical device of any one of items 2627-2853 wherein the vagus nerve stimulator is adapted for treating angina pectoris.

2856. The medical device of any one of items 2627-2853 wherein the vagus nerve stimulator is adapted for treating atrial tachycardia.

2857. The medical device of any one of items 2627-2853 wherein the vagus nerve stimulator is adapted for treating atrial flutter.

2858. The medical device of any one of items 2627-2853 wherein the vagus nerve stimulator is adapted for treating arterial fibrillation.

2859. The medical device of any one of items 2627-2853 wherein the vagus nerve stimulator is arrhythmias that result in low cardiac output.

2860. The medical device of any one of items 2627-2853 wherein the vagus nerve stimulator comprises a programmable pulse generator.

2861. A medical device, comprising an electrical lead (i.e., an electrical device) and an anti-scarring agent or a composition comprising an anti-scarring agent, wherein the agent inhibits scarring between the medical device and the host into which the medical device is implanted.

2862. The medical device of item 2861 wherein the agent is an adensosine A2A receptor antagonist.

2863. The medical device of item 2861 wherein the agent is an AKT inhibitor.

2864. The medical device of item 2861 wherein the agent is an alpha 2 integrin antagonist, wherein the alpha 2 integrin antagonist is Pharmaprojects No. 5754 (Merck KgaA).

2865. The medical device of item 2861 wherein the agent is an alpha 4 integrin antagonist.

2866. The medical device of item 2861 wherein the agent is an alpha 7 nicotinic receptor agonist.

2867. The medical device of item 2861 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

2868. The medical device of item 2861 wherein the agent is an apoptosis antagonist.

2869. The medical device of item 2861 wherein the agent is an apoptosis activator.

2870. The medical device of item 2861 wherein the agent is a beta 1 integrin antagonist.

2871. The medical device of item 2861 wherein the agent is a beta tubulin inhibitor.

2872. The medical device of item 2861 wherein the agent is a blocker of enzyme production in Hepatitis C.

2873. The medical device of item 2861 wherein the agent is a Bruton's tyrosine kinase inhibitor.

2874. The medical device of item 2861 wherein the agent is a calcineurin inhibitor.

2875. The medical device of item 2861 wherein the agent is a caspase 3 inhibitor.

2876. The medical device of item 2861 wherein the agent is a CC chemokine receptor antagonist.

2877. The medical device of item 2861 wherein the agent is a cell cycle inhibitor selected from the group consisting of SNS-595 (Sunesis), synthadotin, KRX-0403, and an analogue or derivative thereof.

2878. The medical device of item 2861 wherein the agent is a cathepsin B inhibitor.

2879. The medical device of item 2861 wherein the agent is a cathepsin K inhibitor, wherein the cathepsin K inhibitor is 462795 (GlaxoSmithKline), INPL-022-D6 (Amura Therapeutics), or an analogue or derivative thereof.

2880. The medical device of item 2861 wherein the agent is a cathepsin L inhibitor.

2881. The medical device of item 2861 wherein the agent is a CD40 antagonist

2882. The medical device of item 2861 wherein the agent is a chemokine receptor agonist.

2883. The medical device of item 2861 wherein the agent is a chymase inhibitor.

2884. The medical device of item 2861 wherein the agent is a collagenase antagonist.

2885. The medical device of item 2861 wherein the agent is a CXCR antagonist.

2886. The medical device of item 2861 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK11CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

2887. The medical device of item 2861 wherein the agent is a cyclooxygenase 1 inhibitor.

2888. The medical device of item 2861 wherein the agent is a DHFR inhibitor.

2889. The medical device of item 2861 wherein the agent is a dual integrin inhibitor.

2890. The medical device of item 2861 wherein the agent is an elastase inhibitor.

2891. The medical device of item 2861 wherein the agent is an elongation factor-1 alpha inhibitor.

2892. The medical device of item 2861 wherein the agent is an endothelial growth factor antagonist.

2893. The medical device of item 2861 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

2894. The medical device of item 2861 wherein the agent is an endotoxin antagonist.

2895. The medical device of item 2861 wherein the agent is an epothilone and tubulin binder.

2896. The medical device of item 2861 wherein the agent is an estrogen receptor antagonist.

2897. The medical device of item 2861 wherein the agent is an FGF inhibitor.

2898. The medical device of item 2861 wherein the agent is a farnexyl transferase inhibitor.

2899. The medical device of item 2861 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

2900. The medical device of item 2861 wherein the agent is an FLT-3 kinase inhibitor.

2901. The medical device of item 2861 wherein the agent is an FGF receptor kinase inhibitor.

2902. The medical device of item 2861 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

2903. The medical device of item 2861 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

2904. The medical device of item 2861 wherein the agent is a histone deacetylase inhibitor.

2905. The medical device of item 2861 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

2906. The medical device of item 2861 wherein the agent is an ICAM inhibitor.

2907. The medical device of item 2861 wherein the agent is an IL, ICE and IRAK antagonist, wherein the antagonist is a CJ-14877, CP-424174 (Pfizer), NF-61 (Negma-Lerads), and an analogue or derivative thereof.

2908. The medical device of item 2861 wherein the agent is an IL-2 inhibitor.

2909. The medical device of item 2861 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

2910. The medical device of item 2861 wherein the agent is an IMPDH (inosine monophosphate).

2911. The medical device of item 2861 wherein the agent is an integrin antagonist.

2912. The medical device of item 2861 wherein the agent is an interleukin antagonist.

2913. The medical device of item 2861 wherein the agent is an inhibitor of type III receptor tyrosine kinase.

2914. The medical device of item 2861 wherein the agent is an irreversible inhibitor of enzyme methionine aminopeptidase type 2.

2915. The medical device of item 2861 wherein the agent is an isozyme selective delta protein kinase C inhibitor.

2916. The medical device of item 2861 wherein the agent a JAK3 enzyme inhibitor.

2917. The medical device of item 2861 wherein the agent is a JNK inhibitor.

2918. The medical device of item 2861 wherein the agent is a kinase inhibitor

2919. The medical device of item 2861 wherein the agent is kinesin antagonist.

2920. The medical device of item 2861 wherein the agent is a kinesin antagonist.

2921. The medical device of item 2861 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

2922. The medical device of item 2861 wherein the agent is an MAP kinase inhibitor.

2923. The medical device of item 2861 wherein the agent is a matrix metalloproteinase inhibitor.

2924. The medical device of item 2861 wherein the agent is an MCP-CCR2 inhibitor.

2925. The medical device of item 2861 wherein the agent is an mTOR inhibitor.

2926. The medical device of item 2861 wherein the agent is an mTOR kinase inhibitor.

2927. The medical device of item 2861 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

2928. The medical device of item 2861 wherein the agent is an MIF inhibitor.

2929. The medical device of item 2861 wherein the agent is an MMP inhibitor.

2930. The medical device of item 2861 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

2931. The medical device of item 2861 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

2932. The medical device of item 2861 wherein the agent is a nitric oxide agonist.

2933. The medical device of item 2861 wherein the agent is an ornithine decarboxylase inhibitor.

2934. The medical device of item 2861 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

2935. The medical device of item 2861 wherein the agent is a palmitoyl-protein thioesterase inhibitor.

2936. The medical device of item 2861 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

2937. The medical device of item 2861 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-154), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

2938. The medical device of item 2861 wherein the agent is a phosphatase inhibitor.

2939. The medical device of item 2861 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

2940. The medical device of item 2861 wherein the agent is a PKC inhibitor.

2941. The medical device of item 2861 wherein the agent is a platelet activating factor antagonist.

2942. The medical device of item 2861 wherein the agent is a platelet-derived growth factor receptor kinase inhibitor.

2943. The medical device of item 2861 wherein the agent is a prolyl hydroxylase inhibitor.

2944. The medical device of item 2861 wherein the agent is a polymorphonuclear neutrophil inhibitor.

2945. The medical device of item 2861 wherein the agent is a protein kinase B inhibitor.

2946. The medical device of item 2861 wherein the agent is a protein kinase C stimulant.

2947. The medical device of item 2861 wherein the agent is a purine nucleoside analogue.

2948. The medical device of item 2861 wherein the agent is a purinoreceptor P2X antagonist.

2949. The medical device of item 2861 wherein the agent is a Raf kinase inhibitor.

2950. The medical device of item 2861 wherein the agent is a reversible inhibitor of ErbB1 and ErbB2.

2951. The medical device of item 2861 wherein the agent is a ribonucleoside triphosphate reductase inhibitor.

2952. The medical device of item 2861 wherein the agent is an SDF-1 antagonist.

2953. The medical device of item 2861 wherein the agent is a sheddase inhibitor.

2954. The medical device of item 2861 wherein the agent is an SRC inhibitor.

2955. The medical device of item 2861 wherein the agent is a stromelysin inhibitor.

2956. The medical device of item 2861 wherein the agent is an Syk kinase inhibitor.

2957. The medical device of item 2861 wherein the agent is a telomerase inhibitor.

2958. The medical device of item 2861 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

2959. The medical device of item 2861 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

2960. The medical device of item 2861 wherein the agent is a Toll receptor inhibitor.

2961. The medical device of item 2861 wherein the agent is a tubulin antagonist.

2962. The medical device of item 2861 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

2963. The medical device of item 2861 wherein the agent is a VEGF inhibitor.

2964. The medical device of item 2861 wherein the agent is a vitamin D receptor agonist.

2965. The medical device of item 2861 wherein the agent is ZD-6474 (an angiogenesis inhibitor).

2966. The medical device of item 2861 wherein the agent is AP-23573 (an mTOR inhibitor).

2967. The medical device of item 2861 wherein the agent is synthadotin (a tubulin antagonist).

2968. The medical device of item 2861 wherein the agent is S-0885 (a collagenase inhibitor).

2969. The medical device of item 2861 wherein the agent is aplidine (an elongation factor-1 alpha inhibitor).

2970. The medical device of item 2861 wherein the agent is ixabepilone (an epithilone).

2971. The medical device of item 2861 wherein the agent is IDN-5390 (an angiogenesis inhibitor).

2972. The medical device of item 2861 wherein the agent is SB-2723005 (an angiogenesis inhibitor).

2973. The medical device of item 2861 wherein the agent is ABT-518 (an angiogenesis inhibitor).

2974. The medical device of item 2861 wherein the agent is combretastatin (an angiogenesis inhibitor).

2975. The medical device of item 2861 wherein the agent is anecortave acetate (an angiogenesis inhibitor).

2976. The medical device of item 2861 wherein the agent is SB-715992 (a kinesin antagonist).

2977. The medical device of item 2861 wherein the agent is temsirolimus (an mTOR inhibitor).

2978. The medical device of item 2861 wherein the agent is adalimumab (a TNFα antagonist).

2979. The medical device of item 2861, further comprising a coating, wherein the coating comprises the anti-scarring agent and a polymer.

2980. The medical device of item 2861, further comprising a coating, wherein the coating comprises the anti-scarring agent.

2981. The medical device of item 2861, further comprising a coating, wherein the coating is disposed on a surface of the electrical device.

2982. The medical device of item 2861, further comprising a coating, wherein the coating directly contacts the electrical device.

2983. The medical device of item 2861, further comprising a coating, wherein the coating indirectly contacts the electrical device.

2984. The medical device of item 2861, further comprising a coating, wherein the coating partially covers the electrical device.

2985. The medical device of item 2861, further comprising a coating, wherein the coating completely covers the electrical device.

2986. The medical device of item 2861, further comprising a coating, wherein the coating is a uniform coating.

2987. The medical device of item 2861, further comprising a coating, wherein the coating is a non-uniform coating.

2988. The medical device of item 2861, further comprising a coating, wherein the coating is a discontinuous coating.

2989. The medical device of item 2861, further comprising a coating, wherein the coating is a patterned coating.

2990. The medical device of item 2861, further comprising a coating, wherein the coating has a thickness of 100 μm or less.

2991. The medical device of item 2861, further comprising a coating, wherein the coating has a thickness of 10 μm or less.

2992. The medical device of item 2861, further comprising a coating, wherein the coating adheres to the surface of the electrical device upon deployment of the medical device.

2993. The medical device of item 2861, further comprising a coating, wherein the coating is stable at room temperature for a period of 1 year.

2994. The medical device of item 2861, further comprising a coating, wherein the anti-scarring agent is present in the coating in an amount ranging between about 0.0001% to about 1% by weight.

2995. The medical device of item 2861, further comprising a coating, wherein the anti-scarring agent is present in the coating in an amount ranging between about 1% to about 10% by weight.

2996. The medical device of item 2861, further comprising a coating, wherein the anti-scarring agent is present in the coating in an amount ranging between about 10% to about 25% by weight.

2997. The medical device of item 2861, further comprising a coating, wherein the anti-scarring agent is present in the coating in an amount ranging between about 25% to about 70% by weight.

2998. The medical device of item 2861, further comprising a coating, wherein the coating further comprises a polymer.

2999. The medical device of item 2861, further comprising a first coating having a first composition and the second coating having a second composition.

3000. The medical device of item 2861, further comprising a first coating having a first composition and the second coating having a second composition, wherein the first composition and the second composition are different.

3001. The medical device of item 2861, further comprising a polymer.

3002. The medical device of item 2861, further comprising a polymeric carrier.

3003. The medical device of item 2861, further comprising a polymeric carrier, wherein the polymeric carrier comprises a copolymer.

3004. The medical device of item 2861, further comprising a polymeric carrier, wherein the polymeric carrier comprises a block copolymer.

3005. The medical device of item 2861, further comprising a polymeric carrier, wherein the polymeric carrier comprises a random copolymer.

3006. The medical device of item 2861, further comprising a polymeric carrier, wherein the polymeric carrier comprises a biodegradable polymer.

3007. The medical device of item 2861, further comprising a polymeric carrier, wherein the polymeric carrier comprises a non-biodegradable polymer.

3008. The medical device of item 2861, further comprising a polymeric carrier, wherein the polymeric carrier comprises a hydrophilic polymer.

3009. The medical device of item 2861, further comprising a polymeric carrier, wherein the polymeric carrier comprises a hydrophobic polymer.

3010. The medical device of item 2861, further comprising a polymeric carrier, wherein the polymeric carrier comprises a polymer having hydrophilic domains.

3011. The medical device of item 2861, further comprising a polymeric carrier, wherein the polymeric carrier comprises a polymer having hydrophobic domains.

3012. The medical device of item 2861, further comprising a polymeric carrier, wherein the polymeric carrier comprises a non-conductive polymer.

3013. The medical device of item 2861, further comprising a polymeric carrier, wherein the polymeric carrier comprises an elastomer.

3014. The medical device of item 2861, further comprising a polymeric carrier, wherein the polymeric carrier comprises a hydrogel.

3015. The medical device of item 2861, further comprising a polymeric carrier, wherein the polymeric carrier comprises a silicone polymer.

3016. The medical device of item 2861, further comprising a polymeric carrier, wherein the polymeric carrier comprises a hydrocarbon polymer.

3017. The medical device of item 2861, further comprising a polymeric carrier, wherein the polymeric carrier comprises a styrene-derived polymer.

3018. The medical device of item 2861, further comprising a polymeric carrier, wherein the polymeric carrier comprises a butadiene polymer.

3019. The medical device of item 2861, further comprising a polymeric carrier, wherein the polymeric carrier comprises a macromer.

3020. The medical device of item 2861, further comprising a polymeric carrier, wherein the polymeric carrier comprises a poly(ethylene glycol) polymer.

3021. The medical device of item 2861, further comprising a polymeric carrier, wherein the polymeric carrier comprises an amorphous polymer.

3022. The medical device of item 2861, further comprising a lubricious coating.

3023. The medical device of item 2861 wherein the anti-scarring agent is located within pores or holes of the electrical device.

3024. The medical device of item 2861 wherein the anti-scarring agent is located within a channel, lumen, or divet of the electrical device.

3025. The medical device of item 2861, further comprising a second pharmaceutically active agent.

3026. The medical device of item 2861, further comprising an anti-inflammatory agent.

3027. The medical device of item 2861, further comprising an agent that inhibits infection.

3028. The medical device of item 2861, further comprising an agent that inhibits infection, wherein the agent is an anthracycline.

3029. The medical device of item 2861, further comprising an agent that inhibits infection, wherein the agent is doxorubicin.

3030. The medical device of item 2861, further comprising an agent that inhibits infection, wherein the agent is mitoxantrone.

3031. The medical device of item 2861, further comprising an agent that inhibits infection, wherein the agent is a fluoropyrimidine.

3032. The medical device of item 2861, further comprising an agent that inhibits infection, wherein the agent is 5-fluorouracil (5-FU).

3033. The medical device of item 2861, further comprising an agent that inhibits infection, wherein the agent is a folic acid antagonist.

3034. The medical device of item 2861, further comprising an agent that inhibits infection, wherein the agent is methotrexate.

3035. The medical device of item 2861, further comprising an agent that inhibits infection, wherein the agent is a podophylotoxin.

3036. The medical device of item 2861, further comprising an agent that inhibits infection, wherein the agent is etoposide.

3037. The medical device of item 2861, further comprising an agent that inhibits infection, wherein the agent is a camptothecin.

3038. The medical device of item 2861, further comprising an agent that inhibits infection, wherein the agent is a hydroxyurea.

3039. The medical device of item 2861, further comprising an agent that inhibits infection, wherein the agent is a platinum complex.

3040. The medical device of item 2861, further comprising an agent that inhibits infection, wherein the agent is cisplatin.

3041. The medical device of item 2861, further comprising an anti-thrombotic agent.

3042. The medical device of item 2861, further comprising a visualization agent.

3043. The medical device of item 2861, further comprising a visualization agent, wherein the visualization agent is a radiopaque material, wherein the radiopaque material comprises a metal, a halogenated compound, or a barium containing compound.

3044. The medical device of item 2861, further comprising a visualization agent, wherein the visualization agent is a radiopaque material, wherein the radiopaque material comprises barium, tantalum, or technetium.

3045. The medical device of item 2861, further comprising a visualization agent, wherein the visualization agent is a MRI responsive material.

3046. The medical device of item 2861, further comprising a visualization agent, wherein the visualization agent comprises a gadolinium chelate.

3047. The medical device of item 2861, further comprising a visualization agent, wherein the visualization agent comprises iron, magnesium, manganese, copper, or chromium.

3048. The medical device of item 2861, further comprising a visualization agent, wherein the visualization agent comprises an iron oxide compound.

3049. The medical device of item 2861, further comprising a visualization agent, wherein the visualization agent comprises a dye, pigment, or colorant.

3050. The medical device of item 2861, further comprising an echogenic material.

3051. The medical device of item 2861, further comprising an echogenic material, wherein the echogenic material is in the form of a coating.

3052. The medical device of item 2861 wherein the device is sterile.

3053. The medical device of item 2861 wherein the anti-scarring agent inhibits adhesion between the medical device and a host into which the medical device is implanted.

3054. The medical device of item 2861 wherein the medical device delivers the anti-scarring agent locally to tissue proximate to the medical device.

3055. The medical device of item 2861 wherein the anti-scarring agent is released into tissue in the vicinity of the medical device after deployment of the medical device.

3056. The medical device of item 2861 wherein the anti-scarring agent is released into tissue in the vicinity of the medical device after deployment of the medical device, wherein the tissue is connective tissue.

3057. The medical device of item 2861 wherein the anti-scarring agent is released into tissue in the vicinity of the medical device after deployment of the medical device, wherein the tissue is muscle tissue.

3058. The medical device of item 2861 wherein the anti-scarring agent is released into tissue in the vicinity of the medical device after deployment of the medical device, wherein the tissue is nerve tissue.

3059. The medical device of item 2861 wherein the anti-scarring agent is released into tissue in the vicinity of the medical device after deployment of the medical device, wherein the tissue is epithelium tissue.

3060. The medical device of item 2861 wherein the anti-scarring agent is released in effective concentrations from the medical device over a period ranging from the time of deployment of the medical device to about 1 year.

3061. The medical device of item 2861 wherein the anti-scarring agent is released in effective concentrations from the medical device over a period ranging from about 1 month to 6 months.

3062. The medical device of item 2861 wherein the anti-scarring agent is released in effective concentrations from the medical device over a period ranging from about 1-90 days.

3063. The medical device of item 2861 wherein the anti-scarring agent is released in effective concentrations from the medical device at a constant rate.

3064. The medical device of item 2861 wherein the anti-scarring agent is released in effective concentrations from the medical device at an increasing rate.

3065. The medical device of item 2861 wherein the anti-scarring agent is released in effective concentrations from the medical device at a decreasing rate.

3066. The medical device of item 2861 wherein the anti-scarring agent is released in effective concentrations from the composition comprising the anti-scarring agent by diffusion over a period ranging from the time of deployment of the medical device to about 90 days.

3067. The medical device of item 2861 wherein the anti-scarring agent is released in effective concentrations from the composition comprising the anti-scarring agent by erosion of the composition over a period ranging from the time of deployment of the medical device to about 90 days.

3068. The medical device of item 2861 wherein the device comprises about 0.01 μg to about 10 μg of the anti-scarring agent.

3069. The medical device of item 2861 wherein the device comprises about 10 μg to about 10 mg of the anti-scarring agent

3070. The medical device of item 2861 wherein the device comprises about 10 mg to about 250 mg of the anti-scarring agent.

3071. The medical device of item 2861 wherein the device comprises about 250 mg to about 1000 mg of the anti-scarring agent.

3072. The medical device of item 2861 wherein the device comprises about 1000 mg to about 2500 mg of the anti-scarring agent.

3073. The medical device of item 2861 wherein a surface of the device comprises less than 0.01 μg of the anti-scarring agent per mm² of device surface to which the anti-scarring agent is applied.

3074. The medical device of item 2861 wherein a surface of the device comprises about 0.01 μg to about 1 μg of the anti-scarring agent per mm² of device surface to which the anti-scarring agent is applied.

3075. The medical device of item 2861 wherein a surface of the device comprises about 1 μg to about 10 μg of the anti-scarring agent per mm² of device surface to which the anti-scarring agent is applied.

3076. The medical device of item 2861 wherein a surface of the device comprises about 10 μg to about 250 μg of the anti-scarring agent per mm² of device surface to which the anti-scarring agent is applied.

3077. The medical device of item 2861 wherein a surface of the device comprises about 250 μg to about 1000 μg of the anti-scarring agent of anti-scarring agent per mm² of device surface to which the anti-scarring agent is applied.

3078. The medical device of item 2861 wherein a surface of the device comprises about 1000 μg to about 2500 μg of the anti-scarring agent per mm² of device surface to which the anti-scarring agent is applied.

3079. The medical device of item 2861 wherein the agent or the composition is affixed to the electrical device.

3080. The medical device of item 2861 wherein the agent or the composition is covalently attached to the electrical device.

3081. The medical device of item 2861 wherein the agent or the composition is non-covalently attached to the electrical device.

3082. The medical device of item 2861 further comprising a coating that absorbs the agent or the composition.

3083. The medical device of item 2861 wherein the electrical device is interweaved with a thread composed of, or coated with, the agent or the composition.

3084. The medical device of item 2861 wherein a portion of the electrical device is covered with a sleeve that contains the agent or the composition.

3085. The medical device of item 2861 wherein the electrical device is completely covered with a sleeve that contains the agent or the composition.

3086. The medical device of item 2861 wherein a portion of the electrical device is covered with a mesh that contains the agent or the composition.

3087. The medical device of item 2861 wherein the electrical device is completely covered with a mesh that contains the agent or the composition.

3088. The medical device of any one of items 2861-3087 wherein the electrical lead comprises a connector assembly, a conductor and an electrode.

3089. The medical device of any one of items 2861-3087 wherein the electrical lead is unipolar.

3090. The medical device of any one of items 2861-3087 wherein the electrical lead is bipolar.

3091. The medical device of any one of items 2861-3087 wherein the electrical lead is tripolar.

3092. The medical device of any one of items 2861-3087 wherein the electrical lead is quadripolar.

3093. The medical device of any one of items 2861-3087 wherein the electrical lead comprises an insulating sheath.

3094. The medical device of any one of items 2861-3087 wherein the electrical lead is a medical lead.

3095. The medical device of any one of items 2861-3087 wherein the electrical lead is a cardiac lead.

3096. The medical device of any one of items 2861-3087 wherein the electrical lead is a pacer lead.

3097. The medical device of any one of items 2861-3087 wherein the electrical lead is a pacing lead.

3098. The medical device of any one of items 2861-3087 wherein the electrical lead is a pacemaker lead.

3099. The medical device of any one of items 2861-3087 wherein the electrical lead is an endocardial lead.

3100. The medical device of any one of items 2861-3087 wherein the electrical lead is an endocardial pacing lead.

3101. The medical device of any one of items 2861-3087 wherein the electrical lead is a cardioversion lead.

3102. The medical device of any one of items 2861-3087 wherein the electrical lead is an epicardial lead.

3103. The medical device of any one of items 2861-3087 wherein the electrical lead is an epicardial defibrillator lead.

3104. The medical device of any one of items 2861-3087 wherein the electrical lead is a patch defibrillator.

3105. The medical device of any one of items 2861-3087 wherein the electrical lead is a patch lead.

3106. The medical device of any one of items 2861-3087 wherein the electrical lead is an electrical patch.

3107. The medical device of any one of items 2861-3087 wherein the electrical lead is a transvenous lead.

3108. The medical device of any one of items 2861-3087 wherein the electrical lead is an active fixation lead.

3109. The medical device of any one of items 2861-3087 wherein the electrical lead is a passive fixation lead.

3110. The medical device of any one of items 2861-3087 wherein the electrical lead is a sensing lead.

3111. The medical device of any one of items 2861-3087 wherein the electrical lead is expandable.

3112. The medical device of any one of items 2861-3087 wherein the electrical lead has a coil configuration.

3113. The medical device of any one of items 2861-3087 wherein the electrical lead has an active fixation element for attachment to host tissue.

3114. A medical device, comprising a neurostimulator (i.e., an electrical device) and an anti-scarring agent or a composition comprising an anti-scarring agent, wherein the agent inhibits scarring between the medical device and the host into which the medical device is implanted.

3115. The medical device of item 3114 wherein the agent is an adensosine A2A receptor antagonist.

3116. The medical device of item 3114 wherein the agent is an AKT inhibitor.

3117. The medical device of item 3114 wherein the agent is an alpha 2 integrin antagonist, wherein the alpha 2 integrin antagonist is Pharmaprojects No. 5754 (Merck KgaA).

3118. The medical device of item 3114 wherein the agent is an alpha 4 integrin antagonist.

3119. The medical device of item 3114 wherein the agent is an alpha 7 nicotinic receptor agonist.

3120. The medical device of item 3114 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

3121. The medical device of item 3114 wherein the agent is an apoptosis antagonist.

3122. The medical device of item 3114 wherein the agent is an apoptosis activator.

3123. The medical device of item 3114 wherein the agent is a beta 1 integrin antagonist.

3124. The medical device of item 3114 wherein the agent is a beta tubulin inhibitor.

3125. The medical device of item 3114 wherein the agent is a blocker of enzyme production in Hepatitis C.

3126. The medical device of item 3114 wherein the agent is a Bruton's tyrosine kinase inhibitor.

3127. The medical device of item 3114 wherein the agent is a calcineurin inhibitor.

3128. The medical device of item 3114 wherein the agent is a caspase 3 inhibitor.

3129. The medical device of item 3114 wherein the agent is a CC chemokine receptor antagonist.

3130. The medical device of item 3114 wherein the agent is a cell cycle inhibitor selected from the group consisting of SNS-595 (Sunesis), synthadotin, KRX-0403, and an analogue or derivative thereof.

3131. The medical device of item 3114 wherein the agent is a cathepsin B inhibitor.

3132. The medical device of item 3114 wherein the agent is a cathepsin K inhibitor, wherein the cathepsin K inhibitor is 462795 (GlaxoSmithKline), INPL-022-D6 (Amura Therapeutics), or an analogue or derivative thereof.

3133. The medical device of item 3114 wherein the agent is a cathepsin L inhibitor.

3134. The medical device of item 3114 wherein the agent is a CD40 antagonist.

3135. The medical device of item 3114 wherein the agent is a chemokine receptor agonist.

3136. The medical device of item 3114 wherein the agent is a chymase inhibitor.

3137. The medical device of item 3114 wherein the agent is a collagenase antagonist.

3138. The medical device of item 3114 wherein the agent is a CXCR antagonist.

3139. The medical device of item 3114 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

3140. The medical device of item 3114 wherein the agent is a cyclooxygenase 1 inhibitor.

3141. The medical device of item 3114 wherein the agent is a DHFR inhibitor.

3142. The medical device of item 3114 wherein the agent is a dual integrin inhibitor.

3143. The medical device of item 3114 wherein the agent is an elastase inhibitor.

3144. The medical device of item 3114 wherein the agent is an elongation factor-1 alpha inhibitor.

3145. The medical device of item 3114 wherein the agent is an endothelial growth factor antagonist.

3146. The medical device of item 3114 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

3147. The medical device of item 3114 wherein the agent is an endotoxin antagonist.

3148. The medical device of item 3114 wherein the agent is an epothilone and tubulin binder.

3149. The medical device of item 3114 wherein the agent is an estrogen receptor antagonist.

3150. The medical device of item 3114 wherein the agent is an FGF inhibitor.

3151. The medical device of item 3114 wherein the agent is a farnexyl transferase inhibitor.

3152. The medical device of item 3114 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

3153. The medical device of item 3114 wherein the agent is an FLT-3 kinase inhibitor.

3154. The medical device of item 3114 wherein the agent is an FGF receptor kinase inhibitor.

3155. The medical device of item 3114 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

3156. The medical device of item 3114 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

3157. The medical device of item 3114 wherein the agent is a histone deacetylase inhibitor.

3158. The medical device of item 3114 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

3159. The medical device of item 3114 wherein the agent is an ICAM inhibitor.

3160. The medical device of item 3114 wherein the agent is an IL, ICE and IRAK antagonist, wherein the antagonist is a CJ-14877, CP-424174 (Pfizer), NF-61 (Negma-Lerads), and an analogue or derivative thereof.

3161. The medical device of item 3114 wherein the agent is an IL-2 inhibitor.

3162. The medical device of item 3114 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

3163. The medical device of item 3114 wherein the agent is an IMPDH (inosine monophosphate).

3164. The medical device of item 3114 wherein the agent is an integrin antagonist.

3165. The medical device of item 3114 wherein the agent is an interleukin antagonist.

3166. The medical device of item 3114 wherein the agent is an inhibitor of type III receptor tyrosine kinase.

3167. The medical device of item 3114 wherein the agent is an irreversible inhibitor of enzyme methionine aminopeptidase type 2.

3168. The medical device of item 3114 wherein the agent is an isozyme selective delta protein kinase C inhibitor.

3169. The medical device of item 3114 wherein the agent a JAK3 enzyme inhibitor.

3170. The medical device of item 3114 wherein the agent is a JNK inhibitor.

3171. The medical device of item 3114 wherein the agent is a kinase inhibitor.

3172. The medical device of item 3114 wherein the agent is kinesin antagonist.

3173. The medical device of item 3114 wherein the agent is a kinesin antagonist.

3174. The medical device of item 3114 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof

3175. The medical device of item 3114 wherein the agent is an MAP kinase inhibitor.

3176. The medical device of item 3114 wherein the agent is a matrix metalloproteinase inhibitor.

3177. The medical device of item 3114 wherein the agent is an MCP-CCR2 inhibitor.

3178. The medical device of item 3114 wherein the agent is an mTOR inhibitor.

3179. The medical device of item 3114 wherein the agent is an mTOR kinase inhibitor.

3180. The medical device of item 3114 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

3181. The medical device of item 3114 wherein the agent is an MIF inhibitor.

3182. The medical device of item 3114 wherein the agent is an MMP inhibitor.

3183. The medical device of item 3114 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

3184. The medical device of item 3114 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

3185. The medical device of item 3114 wherein the agent is a nitric oxide agonist.

3186. The medical device of item 3114 wherein the agent is an ornithine decarboxylase inhibitor.

3187. The medical device of item 3114 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

3188. The medical device of item 3114 wherein the agent is a palmitoyl-protein thioesterase inhibitor.

3189. The medical device of item 3114 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

3190. The medical device of item 3114 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

3191. The medical device of item 3114 wherein the agent is a phosphatase inhibitor.

3192. The medical device of item 3114 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

3193. The medical device of item 3114 wherein the agent is a PKC inhibitor.

3194. The medical device of item 3114 wherein the agent is a platelet activating factor antagonist.

3195. The medical device of item 3114 wherein the agent is a platelet-derived growth factor receptor kinase inhibitor.

3196. The medical device of item 3114 wherein the agent is a prolyl hydroxylase inhibitor.

3197. The medical device of item 3114 wherein the agent is a polymorphonuclear neutrophil inhibitor.

3198. The medical device of item 3114 wherein the agent is a protein kinase B inhibitor.

3199. The medical device of item 3114 wherein the agent is a protein kinase C stimulant.

3200. The medical device of item 3114 wherein the agent is a purine nucleoside analogue.

3201. The medical device of item 3114 wherein the agent is a purinoreceptor P2X antagonist.

3202. The medical device of item 3114 wherein the agent is a Raf kinase inhibitor.

3203. The medical device of item 3114 wherein the agent is a reversible inhibitor of ErbB1 and ErbB2.

3204. The medical device of item 3114 wherein the agent is a ribonucleoside triphosphate reductase inhibitor.

3205. The medical device of item 3114 wherein the agent is an SDF-1 antagonist.

3206. The medical device of item 3114 wherein the agent is a sheddase inhibitor.

3207. The medical device of item 3114 wherein the agent is an SRC inhibitor.

3208. The medical device of item 3114 wherein the agent is a stromelysin inhibitor.

3209. The medical device of item 3114 wherein the agent is an Syk kinase inhibitor.

3210. The medical device of item 3114 wherein the agent is a telomerase inhibitor.

3211. The medical device of item 3114 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

3212. The medical device of item 3114 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

3213. The medical device of item 3114 wherein the agent is a Toll receptor inhibitor.

3214. The medical device of item 3114 wherein the agent is a tubulin antagonist.

3215. The medical device of item 3114 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

3216. The medical device of item 3114 wherein the agent is a VEGF inhibitor.

3217. The medical device of item 3114 wherein the agent is a vitamin D receptor agonist.

3218. The medical device of item 3114 wherein the agent is ZD-6474 (an angiogenesis inhibitor).

3219. The medical device of item 3114 wherein the agent is AP-23573 (an mTOR inhibitor).

3220. The medical device of item 3114 wherein the agent is synthadotin (a tubulin antagonist).

3221. The medical device of item 3114 wherein the agent is S-0885 (a collagenase inhibitor).

3222. The medical device of item 3114 wherein the agent is aplidine (an elongation factor-1 alpha inhibitor).

3223. The medical device of item 3114 wherein the agent is ixabepilone (an epithilone).

3224. The medical device of item 3114 wherein the agent is IDN-5390 (an angiogenesis inhibitor).

3225. The medical device of item 3114 wherein the agent is SB-2723005 (an angiogenesis inhibitor).

3226. The medical device of item 3114 wherein the agent is ABT-518 (an angiogenesis inhibitor).

3227. The medical device of item 3114 wherein the agent is combretastatin (an angiogenesis inhibitor).

3228. The medical device of item 3114 wherein the agent is anecortave acetate (an angiogenesis inhibitor).

3229. The medical device of item 3114 wherein the agent is SB-715992 (a kinesin antagonist).

3230. The medical device of item 3114 wherein the agent is temsirolimus (an mTOR inhibitor).

3231. The medical device of item 3114 wherein the agent is adalimumab (a TNFα antagonist).

3232. The medical device of item 3114, further comprising a coating, wherein the coating comprises the anti-scarring agent and a polymer.

3233. The medical device of item 3114, further comprising a coating, wherein the coating comprises the anti-scarring agent.

3234. The medical device of item 3114, further comprising a coating, wherein the coating is disposed on a surface of the electrical device.

3235. The medical device of item 3114, further comprising a coating, wherein the coating directly contacts the electrical device.

3236. The medical device of item 3114, further comprising a coating, wherein the coating indirectly contacts the electrical device.

3237. The medical device of item 3114, further comprising a coating, wherein the coating partially covers the electrical device.

3238. The medical device of item 3114, further comprising a coating, wherein the coating completely covers the electrical device.

3239. The medical device of item 3114, further comprising a coating, wherein the coating is a uniform coating.

3240. The medical device of item 3114, further comprising a coating, wherein the coating is a non-uniform coating.

3241. The medical device of item 3114, further comprising a coating, wherein the coating is a discontinuous coating.

3242. The medical device of item 3114, further comprising a coating, wherein the coating is a patterned coating.

3243. The medical device of item 3114, further comprising a coating, wherein the coating has a thickness of 100 μm or less.

3244. The medical device of item 3114, further comprising a coating, wherein the coating has a thickness of 10 μm or less.

3245. The medical device of item 3114, further comprising a coating, wherein the coating adheres to the surface of the electrical device upon deployment of the medical device.

3246. The medical device of item 3114, further comprising a coating, wherein the coating is stable at room temperature for a period of 1 year.

3247. The medical device of item 3114, further comprising a coating, wherein the anti-scarring agent is present in the coating in an amount ranging between about 0.0001% to about 1% by weight.

3248. The medical device of item 3114, further comprising a coating, wherein the anti-scarring agent is present in the coating in an amount ranging between about 1% to about 10% by weight.

3249. The medical device of item 3114, further comprising a coating, wherein the anti-scarring agent is present in the coating in an amount ranging between about 10% to about 25% by weight.

3250. The medical device of item 3114, further comprising a coating, wherein the anti-scarring agent is present in the coating in an amount ranging between about 25% to about 70% by weight.

3251. The medical device of item 3114, further comprising a coating, wherein the coating further comprises a polymer.

3252. The medical device of item 3114, further comprising a first coating having a first composition and the second coating having a second composition.

3253. The medical device of item 3114, further comprising a first coating having a first composition and the second coating having a second composition, wherein the first composition and the second composition are different.

3254. The medical device of item 3114, further comprising a polymer.

3255. The medical device of item 3114, further comprising a polymeric carrier.

3256. The medical device of item 3114, further comprising a polymeric carrier, wherein the polymeric carrier comprises a copolymer.

3257. The medical device of item 3114, further comprising a polymeric carrier, wherein the polymeric carrier comprises a block copolymer.

3258. The medical device of item 3114, further comprising a polymeric carrier, wherein the polymeric carrier comprises a random copolymer.

3259. The medical device of item 3114, further comprising a polymeric carrier, wherein the polymeric carrier comprises a biodegradable polymer.

3260. The medical device of item 3114, further comprising a polymeric carrier, wherein the polymeric carrier comprises a non-biodegradable polymer.

3261. The medical device of item 3114, further comprising a polymeric carrier, wherein the polymeric carrier comprises a hydrophilic polymer.

3262. The medical device of item 3114, further comprising a polymeric carrier, wherein the polymeric carrier comprises a hydrophobic polymer.

3263. The medical device of item 3114, further comprising a polymeric carrier, wherein the polymeric carrier comprises a polymer having hydrophilic domains.

3264. The medical device of item 3114, further comprising a polymeric carrier, wherein the polymeric carrier comprises a polymer having hydrophobic domains.

3265. The medical device of item 3114, further comprising a polymeric carrier, wherein the polymeric carrier comprises a non-conductive polymer.

3266. The medical device of item 3114, further comprising a polymeric carrier, wherein the polymeric carrier comprises an elastomer.

3267. The medical device of item 3114, further comprising a polymeric carrier, wherein the polymeric carrier comprises a hydrogel.

3268. The medical device of item 3114, further comprising a polymeric carrier, wherein the polymeric carrier comprises a silicone polymer.

3269. The medical device of item 3114, further comprising a polymeric carrier, wherein the polymeric carrier comprises a hydrocarbon polymer.

3270. The medical device of item 3114, further comprising a polymeric carrier, wherein the polymeric carrier comprises a styrene-derived polymer.

3271. The medical device of item 3114, further comprising a polymeric carrier, wherein the polymeric carrier comprises a butadiene polymer.

3272. The medical device of item 3114, further comprising a polymeric carrier, wherein the polymeric carrier comprises a macromer.

3273. The medical device of item 3114, further comprising a polymeric carrier, wherein the polymeric carrier comprises a poly(ethylene glycol) polymer.

3274. The medical device of item 3114, further comprising a polymeric carrier, wherein the polymeric carrier comprises an amorphous polymer.

3275. The medical device of item 3114, further comprising a lubricious coating.

3276. The medical device of item 3114 wherein the anti-scarring agent is located within pores or holes of the electrical device.

3277. The medical device of item 3114 wherein the anti-scarring agent is located within a channel, lumen, or divet of the electrical device.

3278. The medical device of item 3114, further comprising a second pharmaceutically active agent.

3279. The medical device of item 3114, further comprising an anti-inflammatory agent.

3280. The medical device of item 3114, further comprising an agent that inhibits infection.

3281. The medical device of item 3114, further comprising an agent that inhibits infection, wherein the agent is an anthracycline.

3282. The medical device of item 3114, further comprising an agent that inhibits infection, wherein the agent is doxorubicin.

3283. The medical device of item 3114, further comprising an agent that inhibits infection, wherein the agent is mitoxantrone.

3284. The medical device of item 3114, further comprising an agent that inhibits infection, wherein the agent is a fluoropyrimidine.

3285. The medical device of item 3114, further comprising an agent that inhibits infection, wherein the agent is 5-fluorouracil (5-FU).

3286. The medical device of item 3114, further comprising an agent that inhibits infection, wherein the agent is a folic acid antagonist.

3287. The medical device of item 3114, further comprising an agent that inhibits infection, wherein the agent is methotrexate.

3288. The medical device of item 3114, further comprising an agent that inhibits infection, wherein the agent is a podophylotoxin.

3289. The medical device of item 3114, further comprising an agent that inhibits infection, wherein the agent is etoposide.

3290. The medical device of item 3114, further comprising an agent that inhibits infection, wherein the agent is a camptothecin.

3291. The medical device of item 3114, further comprising an agent that inhibits infection, wherein the agent is a hydroxyurea.

3292. The medical device of item 3114, further comprising an agent that inhibits infection, wherein the agent is a platinum complex.

3293. The medical device of item 3114, further comprising an agent that inhibits infection, wherein the agent is cisplatin.

3294. The medical device of item 3114, further comprising an anti-thrombotic agent.

3295. The medical device of item 3114, further comprising a visualization agent.

3296. The medical device of item 3114, further comprising a visualization agent, wherein the visualization agent is a radiopaque material, wherein the radiopaque material comprises a metal, a halogenated compound, or a barium containing compound.

3297. The medical device of item 3114, further comprising a visualization agent, wherein the visualization agent is a radiopaque material, wherein the radiopaque material comprises barium, tantalum, or technetium.

3298. The medical device of item 3114, further comprising a visualization agent, wherein the visualization agent is a MRI responsive material.

3299. The medical device of item 3114, further comprising a visualization agent, wherein the visualization agent comprises a gadolinium chelate.

3300. The medical device of item 3114, further comprising a visualization agent, wherein the visualization agent comprises iron, magnesium, manganese, copper, or chromium

3301. The medical device of item 3114, further comprising a visualization agent, wherein the visualization agent comprises an iron oxide compound.

3302. The medical device of item 3114, further comprising a visualization agent, wherein the visualization agent comprises a dye, pigment, or colorant.

3303. The medical device of item 3114, further comprising an echogenic material.

3304. The medical device of item 3114, further comprising an echogenic material, wherein the echogenic material is in the form of a coating.

3305. The medical device of item 3114 wherein the device is sterile.

3306. The medical device of item 3114 wherein the anti-scarring agent inhibits adhesion between the medical device and a host into which the medical device is implanted.

3307. The medical device of item 3114 wherein the medical device delivers the anti-scarring agent locally to tissue proximate to the medical device.

3308. The medical device of item 3114 wherein the anti-scarring agent is released into tissue in the vicinity of the medical device after deployment of the medical device.

3309. The medical device of item 3114 wherein the anti-scarring agent is released into tissue in the vicinity of the medical device after deployment of the medical device, wherein the tissue is connective tissue.

3310. The medical device of item 3114 wherein the anti-scarring agent is released into tissue in the vicinity of the medical device after deployment of the medical device, wherein the tissue is muscle tissue.

3311. The medical device of item 3114 wherein the anti-scarring agent is released into tissue in the vicinity of the medical device after deployment of the medical device, wherein the tissue is nerve tissue.

3312. The medical device of item 3114 wherein the anti-scarring agent is released into tissue in the vicinity of the medical device after deployment of the medical device, wherein the tissue is epithelium tissue.

3313. The medical device of item 3114 wherein the anti-scarring agent is released in effective concentrations from the medical device over a period ranging from the time of deployment of the medical device to about 1 year.

3314. The medical device of item 3114 wherein the anti-scarring agent is released in effective concentrations from the medical device over a period ranging from about 1 month to 6 months.

3315. The medical device of item 3114 wherein the anti-scarring agent is released in effective concentrations from the medical device over a period ranging from about 1-90 days.

3316. The medical device of item 3114 wherein the anti-scarring agent is released in effective concentrations from the medical device at a constant rate.

3317. The medical device of item 3114 wherein the anti-scarring agent is released in effective concentrations from the medical device at an increasing rate.

3318. The medical device of item 3114 wherein the anti-scarring agent is released in effective concentrations from the medical device at a decreasing rate.

3319. The medical device of item 3114 wherein the anti-scarring agent is released in effective concentrations from the composition comprising the anti-scarring agent by diffusion over a period ranging from the time of deployment of the medical device to about 90 days.

3320. The medical device of item 3114 wherein the anti-scarring agent is released in effective concentrations from the composition comprising the anti-scarring agent by erosion of the composition over a period ranging from the time of deployment of the medical device to about 90 days.

3321. The medical device of item 3114 wherein the device comprises about 0.01 μg to about 10 μg of the anti-scarring agent.

3322. The medical device of item 3114 wherein the device comprises about 10 μg to about 10 mg of the anti-scarring agent.

3323. The medical device of item 3114 wherein the device comprises about 10 mg to about 250 mg of the anti-scarring agent.

3324. The medical device of item 3114 wherein the device comprises about 250 mg to about 1000 mg of the anti-scarring agent.

3325. The medical device of item 3114 wherein the device comprises about 1000 mg to about 2500 mg of the anti-scarring agent.

3326. The medical device of item 3114 wherein a surface of the device comprises less than 0.01 μg of the anti-scarring agent per mm² of device surface to which the anti-scarring agent is applied.

3327. The medical device of item 3114 wherein a surface of the device comprises about 0.01 μg to about 1 μg of the anti-scarring agent per mm² of device surface to which the anti-scarring agent is applied.

3328. The medical device of item 3114 wherein a surface of the device comprises about 1 μg to about 10 μg of the anti-scarring agent per mm² of device surface to which the anti-scarring agent is applied.

3329. The medical device of item 3114 wherein a surface of the device comprises about 10 μg to about 250 μg of the anti-scarring agent per mm² of device surface to which the anti-scarring agent is applied.

3330. The medical device of item 3114 wherein a surface of the device comprises about 250 μg to about 1000 μg of the anti-scarring agent of anti-scarring agent per mm² of device surface to which the anti-scarring agent is applied.

3331. The medical device of item 3114 wherein a surface of the device comprises about 1000 μg to about 2500 μg of the anti-scarring agent per mm² of device surface to which the anti-scarring agent is applied.

3332. The medical device of item 3114 wherein the agent or the composition is affixed to the electrical device.

3333. The medical device of item 3114 wherein the agent or the composition is covalently attached to the electrical device.

3334. The medical device of item 3114 wherein the agent or the composition is non-covalently attached to the electrical device.

3335. The medical device of item 3114 further comprising a coating that absorbs the agent or the composition.

3336. The medical device of item 3114 wherein the electrical device is interweaved with a thread composed of, or coated with, the agent or the composition.

3337. The medical device of item 3114 wherein a portion of the electrical device is covered with a sleeve that contains the agent or the composition.

3338. The medical device of item 3114 wherein the electrical device is completely covered with a sleeve that contains the agent or the composition.

3339. The medical device of item 3114 wherein a portion of the electrical device is covered with a mesh that contains the agent or the composition.

3340. The medical device of item 3114 wherein the electrical device is completely covered with a mesh that contains the agent or the composition.

3341. The medical device of any one of items 3114-3340 wherein the neurostimulator is a spinal cord stimulator.

3342. The medical device of any one of items 3114-3340 wherein the neurostimulator is a brain stimulator.

3343. The medical device of any one of items 3114-3340 wherein the neurostimulator is a vagus nerve stimulator.

3344. The medical device of any one of items 3114-3340 wherein the neurostimulator is a sacral nerve stimulator.

3345. The medical device of any one of items 3114-3340 wherein the neurostimulator is a gastric nerve stimulator.

3346. The medical device of any one of items 3114-3340 wherein the neurostimulator is an auditory nerve stimulator.

3347. The medical device of any one of items 3114-3340 wherein the neurostimulator delivers stimulation to organs.

3348. The medical device of any one of items 3114-3340 wherein the neurostimulator delivers stimulation to bone.

3349. The medical device of any one of items 3114-3340 wherein the neurostimulator delivers stimulation to muscles.

3350. The medical device of any one of items 3114-3340 wherein the neurostimulator delivers stimulation to tissues.

3351. The medical device of any one of items 3114-3340 wherein the neurostimulator is a device for continuous subarachnoid infusion.

3352. The medical device of any one of items 3114-3340 wherein the neurostimulator is an implantable electrode.

3353. The medical device of any one of items 3114-3340 wherein the neurostimulator is an electrical lead.

3354. The medical device of any one of items 3114-3340 wherein the neurostimulator is a simulation catheter lead.

3355. The medical device of any one of items 3114-3340 wherein the neurostimulator is cochlear implant.

3356. The medical device of any one of items 3114-3340 wherein the neurostimulator is a microstimulator.

3357. The medical device of any one of items 3114-3340 wherein the neurostimulator is battery powered.

3358. The medical device of any one of items 3114-3340 wherein the neurostimulator is radio frequency powered.

3359. The medical device of any one of items 3114-3340 wherein the neurostimulator is both battery and radio frequency powered.

3360. The medical device of any one of items 3114-3340 wherein the neurostimulator is adapted for treating or preventing pain.

3361. The medical device of any one of items 3114-3340 wherein the neurostimulator is adapted for treating or preventing epilepsy.

3362. The medical device of any one of items 3114-3340 wherein the neurostimulator is adapted for treating or preventing Parkinson's disease.

3363. The medical device of any one of items 3114-3340 wherein the neurostimulator is adapted for treating or preventing movement disorders.

3364. The medical device of any one of items 3114-3340 wherein the neurostimulator is adapted for treating or preventing obesity.

3365. The medical device of any one of items 3114-3340 wherein the neurostimulator is adapted for treating or preventing depression.

3366. The medical device of any one of items 3114-3340 wherein the neurostimulator is adapted for treating or preventing anxiety.

3367. The medical device of any one of items 3114-3340 wherein the neurostimulator is adapted for treating or preventing hearing loss.

3368. The medical device of any one of items 3114-3340 wherein the neurostimulator is adapted for treating or preventing ulcers.

3369. The medical device of any one of items 3114-3340 wherein the neurostimulator is adapted for treating or preventing deep vein thrombosis.

3370. The medical device of any one of items 3114-3340 wherein the neurostimulator is adapted for treating or preventing muscular atrophy.

3371. The medical device of any one of items 3114-3340 wherein the neurostimulator is adapted for treating or preventing joint stiffness.

3372. The medical device of any one of items 3114-3340 wherein the neurostimulator is adapted for treating or preventing muscle spasms.

3373. The medical device of any one of items 3114-3340 wherein the neurostimulator is adapted for treating or preventing osteoporosis.

3374. The medical device of any one of items 3114-3340 wherein the neurostimulator is adapted for treating or preventing scoliosis.

3375. The medical device of any one of items 3114-3340 wherein the neurostimulator is adapted for treating or preventing spinal disc degeneration.

3376. The medical device of any one of items 3114-3340 wherein the neurostimulator is adapted for treating or preventing spinal cord injury.

3377. The medical device of any one of items 3114-3340 wherein the neurostimulator is adapted for treating or preventing urinary dysfunction.

3378. The medical device of any one of items 3114-3340 wherein the neurostimulator is adapted for treating or preventing gastroparesis.

3379. The medical device of any one of items 3114-3340 wherein the neurostimulator is adapted for treating or preventing malignancy.

3380. The medical device of any one of items 3114-3340 wherein the neurostimulator is adapted for treating or preventing arachnoiditis.

3381. The medical device of any one of items 3114-3340 wherein the neurostimulator is adapted for treating or preventing chronic disease.

3382. The medical device of any one of items 3114-3340 wherein the neurostimulator is adapted for treating or preventing migraine.

3383. The medical device of any one of items 3114-3340 wherein the neurostimulator is adapted for treating or preventing sleep disorders.

3384. The medical device of any one of items 3114-3340 wherein the neurostimulator is adapted for treating or preventing dementia.

3385. The medical device of any one of items 3114-3340 wherein the neurostimulator is adapted for treating or preventing Alzheimer's disease.

3386. A medical device, comprising a cardiac rhythm management device (i.e., an electrical device) and an anti-scarring agent or a composition comprising an anti-scarring agent, wherein the agent inhibits scarring between the medical device and the host into which the medical device is implanted.

3387. The medical device of item 3386 wherein the agent is an adensosine A2A receptor antagonist.

3388. The medical device of item 3386 wherein the agent is an AKT inhibitor.

3389. The medical device of item 3386 wherein the agent is an alpha 2 integrin antagonist, wherein the alpha 2 integrin antagonist is Pharmaprojects No. 5754 (Merck KgaA).

3390. The medical device of item 3386 wherein the agent is an alpha 4 integrin antagonist.

3391. The medical device of item 3386 wherein the agent is an alpha 7 nicotinic receptor agonist.

3392. The medical device of item 3386 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

3393. The medical device of item 3386 wherein the agent is an apoptosis antagonist.

3394. The medical device of item 3386 wherein the agent is an apoptosis activator.

3395. The medical device of item 3386 wherein the agent is a beta 1 integrin antagonist.

3396. The medical device of item 3386 wherein the agent is a beta tubulin inhibitor.

3397. The medical device of item 3386 wherein the agent is a blocker of enzyme production in Hepatitis C.

3398. The medical device of item 3386 wherein the agent is a Bruton's tyrosine kinase inhibitor.

3399. The medical device of item 3386 wherein the agent is a calcineurin inhibitor.

3400. The medical device of item 3386 wherein the agent is a caspase 3 inhibitor.

3401. The medical device of item 3386 wherein the agent is a CC chemokine receptor antagonist.

3402. The medical device of item 3386 wherein the agent is a cell cycle inhibitor selected from the group consisting of SNS-595 (Sunesis), synthadotin, KRX-0403, and an analogue or derivative thereof.

3403. The medical device of item 3386 wherein the agent is a cathepsin B inhibitor.

3404. The medical device of item 3386 wherein the agent is a cathepsin K inhibitor, wherein the cathepsin K inhibitor is 462795 (GlaxoSmithKline), INPL-022-D6 (Amura Therapeutics), or an analogue or derivative thereof.

3405. The medical device of item 3386 wherein the agent is a cathepsin L inhibitor.

3406. The medical device of item 3386 wherein the agent is a CD40 antagonist.

3407. The medical device of item 3386 wherein the agent is a chemokine receptor agonist.

3408. The medical device of item 3386 wherein the agent is a chymase inhibitor.

3409. The medical device of item 3386 wherein the agent is a collagenase antagonist

3410. The medical device of item 3386 wherein the agent is a CXCR antagonist.

3411. The medical device of item 3386 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

3412. The medical device of item 3386 wherein the agent is a cyclooxygenase 1 inhibitor.

3413. The medical device of item 3386 wherein the agent is a DHFR inhibitor.

3414. The medical device of item 3386 wherein the agent is a dual integrin inhibitor.

3415. The medical device of item 3386 wherein the agent is an elastase inhibitor.

3416. The medical device of item 3386 wherein the agent is an elongation factor-1 alpha inhibitor.

3417. The medical device of item 3386 wherein the agent is an endothelial growth factor antagonist.

3418. The medical device of item 3386 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

3419. The medical device of item 3386 wherein the agent is an endotoxin antagonist.

3420. The medical device of item 3386 wherein the agent is an epothilone and tubulin binder.

3421. The medical device of item 3386 wherein the agent is an estrogen receptor antagonist.

3422. The medical device of item 3386 wherein the agent is an FGF inhibitor.

3423. The medical device of item 3386 wherein the agent is a farnexyl transferase inhibitor.

3424. The medical device of item 3386 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

3425. The medical device of item 3386 wherein the agent is an FLT-3 kinase inhibitor.

3426. The medical device of item 3386 wherein the agent is an FGF receptor kinase inhibitor.

3427. The medical device of item 3386 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

3428. The medical device of item 3386 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

3429. The medical device of item 3386 wherein the agent is a histone deacetylase inhibitor.

3430. The medical device of item 3386 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

3431. The medical device of item 3386 wherein the agent is an ICAM inhibitor.

3432. The medical device of item 3386 wherein the agent is an IL, ICE and IRAK antagonist, wherein the antagonist is a CJ-14877, CP-424174 (Pfizer), NF-61 (Negma-Lerads), and an analogue or derivative thereof.

3433. The medical device of item 3386 wherein the agent is an IL-2 inhibitor.

3434. The medical device of item 3386 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

3435. The medical device of item 3386 wherein the agent is an IMPDH (inosine monophosphate).

3436. The medical device of item 3386 wherein the agent is an integrin antagonist.

3437. The medical device of item 3386 wherein the agent is an interleukin antagonist.

3438. The medical device of item 3386 wherein the agent is an inhibitor of type III receptor tyrosine kinase.

3439. The medical device of item 3386 wherein the agent is an irreversible inhibitor of enzyme methionine aminopeptidase type 2.

3440. The medical device of item 3386 wherein the agent is an isozyme selective delta protein kinase C inhibitor.

3441. The medical device of item 3386 wherein the agent a JAK3 enzyme inhibitor.

3442. The medical device of item 3386 wherein the agent is a JNK inhibitor.

3443. The medical device of item 3386 wherein the agent is a kinase inhibitor.

3444. The medical device of item 3386 wherein the agent is kinesin antagonist.

3445. The medical device of item 3386 wherein the agent is a kinesin antagonist.

3446. The medical device of item 3386 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

3447. The medical device of item 3386 wherein the agent is an MAP kinase inhibitor.

3448. The medical device of item 3386 wherein the agent is a matrix metalloproteinase inhibitor.

3449. The medical device of item 3386 wherein the agent is an MCP-CCR2 inhibitor.

3450. The medical device of item 3386 wherein the agent is an mTOR inhibitor.

3451. The medical device of item 3386 wherein the agent is an mTOR kinase inhibitor.

3452. The medical device of item 3386 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

3453. The medical device of item 3386 wherein the agent is an MIF inhibitor.

3454. The medical device of item 3386 wherein the agent is an MMP inhibitor.

3455. The medical device of item 3386 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

3456. The medical device of item 3386 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

3457. The medical device of item 3386 wherein the agent is a nitric oxide agonist.

3458. The medical device of item 3386 wherein the agent is an ornithine decarboxylase inhibitor.

3459. The medical device of item 3386 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

3460. The medical device of item 3386 wherein the agent is a palmitoyl-protein thioesterase inhibitor.

3461. The medical device of item 3386 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

3462. The medical device of item 3386 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

3463. The medical device of item 3386 wherein the agent is a phosphatase inhibitor.

3464. The medical device of item 3386 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

3465. The medical device of item 3386 wherein the agent is a PKC inhibitor.

3466. The medical device of item 3386 wherein the agent is a platelet activating factor antagonist.

3467. The medical device of item 3386 wherein the agent is a platelet-derived growth factor receptor kinase inhibitor.

3468. The medical device of item 3386 wherein the agent is a prolyl hydroxylase inhibitor.

3469. The medical device of item 3386 wherein the agent is a polymorphonuclear neutrophil inhibitor.

3470. The medical device of item 3386 wherein the agent is a protein kinase B inhibitor.

3471. The medical device of item 3386 wherein the agent is a protein kinase C stimulant.

3472. The medical device of item 3386 wherein the agent is a purine nucleoside analogue.

3473. The medical device of item 3386 wherein the agent is a purinoreceptor P2X antagonist.

3474. The medical device of item 3386 wherein the agent is a Raf kinase inhibitor.

3475. The medical device of item 3386 wherein the agent is a reversible inhibitor of ErbB1 and ErbB2.

3476. The medical device of item 3386 wherein the agent is a ribonucleoside triphosphate reductase inhibitor.

3477. The medical device of item 3386 wherein the agent is an SDF-1 antagonist.

3478. The medical device of item 3386 wherein the agent is a sheddase inhibitor.

3479. The medical device of item 3386 wherein the agent is an SRC inhibitor.

3480. The medical device of item 3386 wherein the agent is a stromelysin inhibitor.

3481. The medical device of item 3386 wherein the agent is an Syk kinase inhibitor.

3482. The medical device of item 3386 wherein the agent is a telomerase inhibitor.

3483. The medical device of item 3386 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

3484. The medical device of item 3386 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

3485. The medical device of item 3386 wherein the agent is a Toll receptor inhibitor.

3486. The medical device of item 3386 wherein the agent is a tubulin antagonist.

3487. The medical device of item 3386 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

3488. The medical device of item 3386 wherein the agent is a VEGF inhibitor.

3489. The medical device of item 3386 wherein the agent is a vitamin D receptor agonist.

3490. The medical device of item 3386 wherein the agent is ZD-6474 (an angiogenesis inhibitor).

3491. The medical device of item 3386 wherein the agent is AP-23573 (an mTOR inhibitor).

3492. The medical device of item 3386 wherein the agent is synthadotin (a tubulin antagonist).

3493. The medical device of item 3386 wherein the agent is S-0885 (a collagenase inhibitor).

3494. The medical device of item 3386 wherein the agent is aplidine (an elongation factor-1 alpha inhibitor).

3495. The medical device of item 3386 wherein the agent is ixabepilone (an epithilone).

3496. The medical device of item 3386 wherein the agent is IDN-5390 (an angiogenesis inhibitor).

3497. The medical device of item 3386 wherein the agent is SB-2723005 (an angiogenesis inhibitor).

3498. The medical device of item 3386 wherein the agent is ABT-518 (an angiogenesis inhibitor).

3499. The medical device of item 3386 wherein the agent is combretastatin (an angiogenesis inhibitor).

3500. The medical device of item 3386 wherein the agent is anecortave acetate (an angiogenesis inhibitor).

3501. The medical device of item 3386 wherein the agent is SB-715992 (a kinesin antagonist).

3502. The medical device of item 3386 wherein the agent is temsirolimus (an mTOR inhibitor).

3503. The medical device of item 3386 wherein the agent is adalimumab (a TNFα antagonist).

3504. The medical device of item 3386, further comprising a coating, wherein the coating comprises the anti-scarring agent and a polymer.

3505. The medical device of item 3386, further comprising a coating, wherein the coating comprises the anti-scarring agent.

3506. The medical device of item 3386, further comprising a coating, wherein the coating is disposed on a surface of the electrical device.

3507. The medical device of item 3386, further comprising a coating, wherein the coating directly contacts the electrical device.

3508. The medical device of item 3386, further comprising a coating, wherein the coating indirectly contacts the electrical device.

3509. The medical device of item 3386, further comprising a coating, wherein the coating partially covers the electrical device.

3510. The medical device of item 3386, further comprising a coating, wherein the coating completely covers the electrical device.

3511. The medical device of item 3386, further comprising a coating, wherein the coating is a uniform coating.

3512. The medical device of item 3386, further comprising a coating, wherein the coating is a non-uniform coating.

3513. The medical device of item 3386, further comprising a coating, wherein the coating is a discontinuous coating.

3514. The medical device of item 3386, further comprising a coating, wherein the coating is a patterned coating.

3515. The medical device of item 3386, further comprising a coating, wherein the coating has a thickness of 100 μm or less.

3516. The medical device of item 3386, further comprising a coating, wherein the coating has a thickness of 10 μm or less.

3517. The medical device of item 3386, further comprising a coating, wherein the coating adheres to the surface of the electrical device upon deployment of the medical device.

3518. The medical device of item 3386, further comprising a coating, wherein the coating is stable at room temperature for a period of 1 year.

3519. The medical device of item 3386, further comprising a coating, wherein the anti-scarring agent is present in the coating in an amount ranging between about 0.0001% to about 1% by weight.

3520. The medical device of item 3386, further comprising a coating, wherein the anti-scarring agent is present in the coating in an amount ranging between about 1% to about 10% by weight.

3521. The medical device of item 3386, further comprising a coating, wherein the anti-scarring agent is present in the coating in an amount ranging between about 10% to about 25% by weight.

3522. The medical device of item 3386, further comprising a coating, wherein the anti-scarring agent is present in the coating in an amount ranging between about 25% to about 70% by weight.

3523. The medical device of item 3386, further comprising a coating, wherein the coating further comprises a polymer.

3524. The medical device of item 3386, further comprising a first coating having a first composition and the second coating having a second composition.

3525. The medical device of item 3386, further comprising a first coating having a first composition and the second coating having a second composition, wherein the first composition and the second composition are different.

3526. The medical device of item 3386, further comprising a polymer.

3527. The medical device of item 3386, further comprising a polymeric carrier.

3528. The medical device of item 3386, further comprising a polymeric carrier, wherein the polymeric carrier comprises a copolymer.

3529. The medical device of item 3386, further comprising a polymeric carrier, wherein the polymeric carrier comprises a block copolymer.

3530. The medical device of item 3386, further comprising a polymeric carrier, wherein the polymeric carrier comprises a random copolymer.

3531. The medical device of item 3386, further comprising a polymeric carrier, wherein the polymeric carrier comprises a biodegradable polymer.

3532. The medical device of item 3386, further comprising a polymeric carrier, wherein the polymeric carrier comprises a non-biodegradable polymer.

3533. The medical device of item 3386, further comprising a polymeric carrier, wherein the polymeric carrier comprises a hydrophilic polymer.

3534. The medical device of item 3386, further comprising a polymeric carrier, wherein the polymeric carrier comprises a hydrophobic polymer.

3535. The medical device of item 3386, further comprising a polymeric carrier, wherein the polymeric carrier comprises a polymer having hydrophilic domains.

3536. The medical device of item 3386, further comprising a polymeric carrier, wherein the polymeric carrier comprises a polymer having hydrophobic domains.

3537. The medical device of item 3386, further comprising a polymeric carrier, wherein the polymeric carrier comprises a non-conductive polymer.

3538. The medical device of item 3386, further comprising a polymeric carrier, wherein the polymeric carrier comprises an elastomer.

3539. The medical device of item 3386, further comprising a polymeric carrier, wherein the polymeric carrier comprises a hydrogel.

3540. The medical device of item 3386, further comprising a polymeric carrier, wherein the polymeric carrier comprises a silicone polymer.

3541. The medical device of item 3386, further comprising a polymeric carrier, wherein the polymeric carrier comprises a hydrocarbon polymer.

3542. The medical device of item 3386, further comprising a polymeric carrier, wherein the polymeric carrier comprises a styrene-derived polymer.

3543. The medical device of item 3386, further comprising a polymeric carrier, wherein the polymeric carrier comprises a butadiene polymer.

3544. The medical device of item 3386, further comprising a polymeric carrier, wherein the polymeric carrier comprises a macromer.

3545. The medical device of item 3386, further comprising a polymeric carrier, wherein the polymeric carrier comprises a poly(ethylene glycol) polymer.

3546. The medical device of item 3386, further comprising a polymeric carrier, wherein the polymeric carrier comprises an amorphous polymer.

3547. The medical device of item 3386, further comprising a lubricious coating.

3548. The medical device of item 3386 wherein the anti-scarring agent is located within pores or holes of the electrical device.

3549. The medical device of item 3386 wherein the anti-scarring agent is located within a channel, lumen, or divet of the electrical device.

3550. The medical device of item 3386, further comprising a second pharmaceutically active agent.

3551. The medical device of item 3386, further comprising an anti-inflammatory agent.

3552. The medical device of item 3386, further comprising an agent that inhibits infection.

3553. The medical device of item 3386, further comprising an agent that inhibits infection, wherein the agent is an anthracycline.

3554. The medical device of item 3386, further comprising an agent that inhibits infection, wherein the agent is doxorubicin.

3555. The medical device of item 3386, further comprising an agent that inhibits infection, wherein the agent is mitoxantrone.

3556. The medical device of item 3386, further comprising an agent that inhibits infection, wherein the agent is a fluoropyrimidine.

3557. The medical device of item 3386, further comprising an agent that inhibits infection, wherein the agent is 5-fluorouracil (5-FU).

3558. The medical device of item 3386, further comprising an agent that inhibits infection, wherein the agent is a folic acid antagonist.

3559. The medical device of item 3386, further comprising an agent that inhibits infection, wherein the agent is methotrexate.

3560. The medical device of item 3386, further comprising an agent that inhibits infection, wherein the agent is a podophylotoxin.

3561. The medical device of item 3386, further comprising an agent that inhibits infection, wherein the agent is etoposide.

3562. The medical device of item 3386, further comprising an agent that inhibits infection, wherein the agent is a camptothecin.

3563. The medical device of item 3386, further comprising an agent that inhibits infection, wherein the agent is a hydroxyurea.

3564. The medical device of item 3386, further comprising an agent that inhibits infection, wherein the agent is a platinum complex.

3565. The medical device of item 3386, further comprising an agent that inhibits infection, wherein the agent is cisplatin.

3566. The medical device of item 3386, further comprising an anti-thrombotic agent

3567. The medical device of item 3386, further comprising a visualization agent.

3568. The medical device of item 3386, further comprising a visualization agent, wherein the visualization agent is a radiopaque material, wherein the radiopaque material comprises a metal, a halogenated compound, or a barium containing compound.

3569. The medical device of item 3386, further comprising a visualization agent, wherein the visualization agent is a radiopaque material, wherein the radiopaque material comprises barium, tantalum, or technetium.

3570. The medical device of item 3386, further comprising a visualization agent, wherein the visualization agent is a MRI responsive material.

3571. The medical device of item 3386, further comprising a visualization agent, wherein the visualization agent comprises a gadolinium chelate.

3572. The medical device of item 3386, further comprising a visualization agent, wherein the visualization agent comprises iron, magnesium, manganese, copper, or chromium.

3573. The medical device of item 3386, further comprising a visualization agent, wherein the visualization agent comprises an iron oxide compound.

3574. The medical device of item 3386, further comprising a visualization agent, wherein the visualization agent comprises a dye, pigment, or colorant.

3575. The medical device of item 3386, further comprising an echogenic material.

3576. The medical device of item 3386, further comprising an echogenic material, wherein the echogenic material is in the form of a coating.

3577. The medical device of item 3386 wherein the device is sterile.

3578. The medical device of item 3386 wherein the anti-scarring agent inhibits adhesion between the medical device and a host into which the medical device is implanted.

3579. The medical device of item 3386 wherein the medical device delivers the anti-scarring agent locally to tissue proximate to the medical device.

3580. The medical device of item 3386 wherein the anti-scarring agent is released into tissue in the vicinity of the medical device after deployment of the medical device.

3581. The medical device of item 3386 wherein the anti-scarring agent is released into tissue in the vicinity of the medical device after deployment of the medical device, wherein the tissue is connective tissue.

3582. The medical device of item 3386 wherein the anti-scarring agent is released into tissue in the vicinity of the medical device after deployment of the medical device, wherein the tissue is muscle tissue.

3583. The medical device of item 3386 wherein the anti-scarring agent is released into tissue in the vicinity of the medical device after deployment of the medical device, wherein the tissue is nerve tissue.

3584. The medical device of item 3386 wherein the anti-scarring agent is released into tissue in the vicinity of the medical device after deployment of the medical device, wherein the tissue is epithelium tissue.

3585. The medical device of item 3386 wherein the anti-scarring agent is released in effective concentrations from the medical device over a period ranging from the time of deployment of the medical device to about 1 year.

3586. The medical device of item 3386 wherein the anti-scarring agent is released in effective concentrations from the medical device over a period ranging from about 1 month to 6 months.

3587. The medical device of item 3386 wherein the anti-scarring agent is released in effective concentrations from the medical device over a period ranging from about 1-90 days.

3588. The medical device of item 3386 wherein the anti-scarring agent is released in effective concentrations from the medical device at a constant rate.

3589. The medical device of item 3386 wherein the anti-scarring agent is released in effective concentrations from the medical device at an increasing rate.

3590. The medical device of item 3386 wherein the anti-scarring agent is released in effective concentrations from the medical device at a decreasing rate.

3591. The medical device of item 3386 wherein the anti-scarring agent is released in effective concentrations from the composition comprising the anti-scarring agent by diffusion over a period ranging from the time of deployment of the medical device to about 90 days.

3592. The medical device of item 3386 wherein the anti-scarring agent is released in effective concentrations from the composition comprising the anti-scarring agent by erosion of the composition over a period ranging from the time of deployment of the medical device to about 90 days.

3593. The medical device of item 3386 wherein the device comprises about 0.01 μg to about 10 μg of the anti-scarring agent.

3594. The medical device of item 3386 wherein the device comprises about 10 μg to about 10 mg of the anti-scarring agent.

3595. The medical device of item 3386 wherein the device comprises about 10 mg to about 250 mg of the anti-scarring agent.

3596. The medical device of item 3386 wherein the device comprises about 250 mg to about 1000 mg of the anti-scarring agent.

3597. The medical device of item 3386 wherein the device comprises about 1000 mg to about 2500 mg of the anti-scarring agent.

3598. The medical device of item 3386 wherein a surface of the device comprises less than 0.01 μg of the anti-scarring agent per mm² of device surface to which the anti-scarring agent is applied.

3599. The medical device of item 3386 wherein a surface of the device comprises about 0.01 μg to about 1 μg of the anti-scarring agent per mm² of device surface to which the anti-scarring agent is applied.

3600. The medical device of item 3386 wherein a surface of the device comprises about 1 μg to about 10 μg of the anti-scarring agent per mm² of device surface to which the anti-scarring agent is applied.

3601. The medical device of item 3386 wherein a surface of the device comprises about 10 μg to about 250 μg of the anti-scarring agent per mm² of device surface to which the anti-scarring agent is applied.

3602. The medical device of item 3386 wherein a surface of the device comprises about 250 μg to about 1000 μg of the anti-scarring agent of anti-scarring agent per mm² of device surface to which the anti-scarring agent is applied.

3603. The medical device of item 3386 wherein a surface of the device comprises about 1000 μg to about 2500 μg of the anti-scarring agent per mm² of device surface to which the anti-scarring agent is applied.

3604. The medical device of item 3386 wherein the agent or the composition is affixed to the electrical device.

3605. The medical device of item 3386 wherein the agent or the composition is covalently attached to the electrical device.

3606. The medical device of item 3386 wherein the agent or the composition is non-covalently attached to the electrical device.

3607. The medical device of item 3386 further comprising a coating that absorbs the agent or the composition.

3608. The medical device of item 3386 wherein the electrical device is interweaved with a thread composed of, or coated with, the agent or the composition.

3609. The medical device of item 3386 wherein a portion of the electrical device is covered with a sleeve that contains the agent or the composition.

3610. The medical device of item 3386 wherein the electrical device is completely covered with a sleeve that contains the agent or the composition.

3611. The medical device of item 3386 wherein a portion of the electrical device is covered with a mesh that contains the agent or the composition.

3612. The medical device of item 3386 wherein the electrical device is completely covered with a mesh that contains the agent or the composition.

3613. The medical device of any one of items 3386-3612 wherein the cardiac rhythm management device is an implantable pulse generator.

3614. The medical device of any one of items 3386-3612 wherein the cardiac rhythm management device is an electrical lead.

3615. The medical device of any one of items 3386-3612 wherein the cardiac rhythm management device is a stimulation lead.

3616. The medical device of any one of items 3386-3612 wherein the cardiac rhythm management device is a simulation catheter lead.

3617. The medical device of any one of items 3386-3612 wherein the cardiac rhythm management device is a microstimulator.

3618. The medical device of any one of items 3386-3612 wherein the cardiac rhythm management device is battery powered.

3619. The medical device of any one of items 3386-3612 wherein the cardiac rhythm management device is radio frequency powered.

3620. The medical device of any one of items 3386-3612 wherein the cardiac rhythm management device is both battery and radio frequency powered.

3621. The medical device of any one of items 3386-3612 wherein the cardiac rhythm management device is a cardiac pacemaker.

3622. The medical device of any one of items 3386-3612 wherein the cardiac rhythm management device is an implantable cardioverter defibrillator system.

3623. The medical device of any one of items 3386-3612 wherein the cardiac rhythm management device is a cardiac lead.

3624. The medical device of any one of items 3386-3612 wherein the cardiac rhythm management device is a pacer lead.

3625. The medical device of any one of items 3386-3612 wherein the cardiac rhythm management device is an endocardial lead.

3626. The medical device of any one of items 3386-3612 wherein the cardiac rhythm management device is a cardioversion/defibrillator lead.

3627. The medical device of any one of items 3386-3612 wherein the cardiac rhythm management device is an epicardial lead.

3628. The medical device of any one of items 3386-3612 wherein the cardiac rhythm management device is an epicardial defibrillator lead.

3629. The medical device of any one of items 3386-3612 wherein the cardiac rhythm management device is a patch defibrillator.

3630. The medical device of any one of items 3386-3612 wherein the cardiac rhythm management device is a patch defibrillator lead.

3631. The medical device of any one of items 3386-3612 wherein the cardiac rhythm management device is an electrical patch.

3632. The medical device of any one of items 3386-3612 wherein the cardiac rhythm management device is a transvenous lead.

3633. The medical device of any one of items 3386-3612 wherein the cardiac rhythm management device is an active fixation lead.

3634. The medical device of any one of items 3386-3612 wherein the cardiac rhythm management device is a passive fixation lead.

3635. The medical device of any one of items 3386-3612 wherein the cardiac rhythm management device is a sensing lead.

3636. The medical device of any one of items 3386-3612 wherein the cardiac rhythm management device is a defibrillator.

3637. The medical device of any one of items 3386-3612 wherein the cardiac rhythm management device is an implantable sensor.

3638. The medical device of any one of items 3386-3612 wherein the cardiac rhythm management device is a left ventricular assist device.

3639. The medical device of any one of items 3386-3612 wherein the cardiac rhythm management device is a pulse generator.

3640. The medical device of any one of items 3386-3612 wherein the cardiac rhythm management device is a patch lead.

3641. The medical device of any one of items 3386-3612 wherein the cardiac rhythm management device is an electrical patch.

3642. The medical device of any one of items 3386-3612 wherein the cardiac rhythm management device is a cardiac stimulator.

3643. The medical device of any one of items 3386-3612 wherein the cardiac rhythm management device is an electrical deviceable sensor.

3644. The medical device of any one of items 3386-3612 wherein the cardiac rhythm management device is an electrical deviceable pump.

3645. The medical device of any one of items 3386-3612 wherein the cardiac rhythm management device is a dural patch.

3646. The medical device of any one of items 3386-3612 wherein the cardiac rhythm management device is a ventricular peritoneal shunt.

3647. The medical device of any one of items 3386-3612 wherein the cardiac rhythm management device is a ventricular atrial shunt.

3648. The medical device of any one of items 3386-3612 wherein the cardiac rhythm management device is adapted for treating or preventing epidural fibrosis post-laminectomy.

3649. The medical device of any one of items 3386-3612 wherein the cardiac rhythm management device is adapted for treating or preventing cardiac rhythm abnormalities.

3650. The medical device of any one of items 3386-3612 wherein the cardiac rhythm management device is adapted for treating or preventing atrial rhythm abnormalities.

3651. The medical device of any one of items 3386-3612 wherein the cardiac rhythm management device is adapted for treating or preventing conduction abnormalities.

3652. The medical device of any one of items 3386-3612 wherein the cardiac rhythm management device is adapted for treating or preventing ventricular rhythm abnormalities.

3653. A method for inhibiting scarring comprising placing an electrical device and an anti-scarring agent or a composition comprising an ant-scarring agent into an animal host, wherein the agent inhibits scarring.

3654. The method of item 3653 wherein the agent is an adensosine A2A receptor antagonist.

3655. The method of item 3653 wherein the agent is an AKT inhibitor.

3656. The method of item 3653 wherein the agent is an alpha 2 integrin antagonist, wherein the alpha 2 integrin antagonist is Pharmaprojects No. 5754 (Merck KgaA).

3657. The method of item 3653 wherein the agent is an alpha 4 integrin antagonist.

3658. The method of item 3653 wherein the agent is an alpha 7 nicotinic receptor agonist.

3659. The method of item 3653 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

3660. The method of item 3653 wherein the agent is an apoptosis antagonist.

3661. The method of item 3653 wherein the agent is an apoptosis activator.

3662. The method of item 3653 wherein the agent is a beta 1 integrin antagonist.

3663. The method of item 3653 wherein the agent is a beta tubulin inhibitor.

3664. The method of item 3653 wherein the agent is a blocker of enzyme production in Hepatitis C.

3665. The method of item 3653 wherein the agent is a Bruton's tyrosine kinase inhibitor.

3666. The method of item 3653 wherein the agent is a calcineurin inhibitor.

3667. The method of item 3653 wherein the agent is a caspase 3 inhibitor.

3668. The method of item 3653 wherein the agent is a CC chemokine receptor antagonist.

3669. The method of item 3653 wherein the agent is a cell cycle inhibitor selected from the group consisting of SNS-595 (Sunesis), synthadotin, KRX-0403, and an analogue or derivative thereof.

3670. The method of item 3653 wherein the agent is a cathepsin B inhibitor.

3671. The method of item 3653 wherein the agent is a cathepsin K inhibitor, wherein the cathepsin K inhibitor is 462795 (GlaxoSmithKline), INPL-022-D6 (Amura Therapeutics), or an analogue or derivative thereof.

3672. The method of item 3653 wherein the agent is a cathepsin L inhibitor.

3673. The method of item 3653 wherein the agent is a CD40 antagonist.

3674. The method of item 3653 wherein the agent is a chemokine receptor agonist.

3675. The method of item 3653 wherein the agent is a chymase inhibitor.

3676. The method of item 3653 wherein the agent is a collagenase antagonist.

3677. The method of item 3653 wherein the agent is a CXCR antagonist.

3678. The method of item 3653 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

3679. The method of item 3653 wherein the agent is a cyclooxygenase 1 inhibitor.

3680. The method of item 3653 wherein the agent is a DHFR inhibitor.

3681. The method of item 3653 wherein the agent is a dual integrin inhibitor.

3682. The method of item 3653 wherein the agent is an elastase inhibitor.

3683. The method of item 3653 wherein the agent is an elongation factor-1 alpha inhibitor.

3684. The method of item 3653 wherein the agent is an endothelial growth factor antagonist.

3685. The method of item 3653 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

3686. The method of item 3653 wherein the agent is an endotoxin antagonist.

3687. The method of item 3653 wherein the agent is an epothilone and tubulin binder.

3688. The method of item 3653 wherein the agent is an estrogen receptor antagonist.

3689. The method of item 3653 wherein the agent is an FGF inhibitor.

3690. The method of item 3653 wherein the agent is a farnexyl transferase inhibitor.

3691. The method of item 3653 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

3692. The method of item 3653 wherein the agent is an FLT-3 kinase inhibitor.

3693. The method of item 3653 wherein the agent is an FGF receptor kinase inhibitor.

3694. The method of item 3653 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

3695. The method of item 3653 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

3696. The method of item 3653 wherein the agent is a histone deacetylase inhibitor.

3697. The method of item 3653 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

3698. The method of item 3653 wherein the agent is an ICAM inhibitor.

3699. The method of item 3653 wherein the agent is an IL, ICE and IRAK antagonist, wherein the antagonist is a CJ-14877, CP-424174 (Pfizer), NF-61 (Negma-Lerads), and an analogue or derivative thereof.

3700. The method of item 3653 wherein the agent is an IL-2 inhibitor.

3701. The method of item 3653 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

3702. The method of item 3653 wherein the agent is an IMPDH (inosine monophosphate).

3703. The method of item 3653 wherein the agent is an integrin antagonist.

3704. The method of item 3653 wherein the agent is an interleukin antagonist.

3705. The method of item 3653 wherein the agent is an inhibitor of type III receptor tyrosine kinase.

3706. The method of item 3653 wherein the agent is an irreversible inhibitor of enzyme methionine aminopeptidase type 2.

3707. The method of item 3653 wherein the agent is an isozyme selective delta protein kinase C inhibitor.

3708. The method of item 3653 wherein the agent a JAK3 enzyme inhibitor.

3709. The method of item 3653 wherein the agent is a JNK inhibitor.

3710. The method of item 3653 wherein the agent is a kinase inhibitor.

3711. The method of item 3653 wherein the agent is kinesin antagonist.

3712. The method of item 3653 wherein the agent is a kinesin antagonist.

3713. The method of item 3653 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

3714. The method of item 3653 wherein the agent is an MAP kinase inhibitor.

3715. The method of item 3653 wherein the agent is a matrix metalloproteinase inhibitor.

3716. The method of item 3653 wherein the agent is an MCP-CCR2 inhibitor.

3717. The method of item 3653 wherein the agent is an mTOR inhibitor.

3718. The method of item 3653 wherein the agent is an mTOR kinase inhibitor.

3719. The method of item 3653 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

3720. The method of item 3653 wherein the agent is an MIF inhibitor.

3721. The method of item 3653 wherein the agent is an MMP inhibitor.

3722. The method of item 3653 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

3723. The method of item 3653 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

3724. The method of item 3653 wherein the agent is a nitric oxide agonist

3725. The method of item 3653 wherein the agent is an ornithine decarboxylase inhibitor.

3726. The method of item 3653 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

3727. The method of item 3653 wherein the agent is a palmitoyl-protein thioesterase inhibitor.

3728. The method of item 3653 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

3729. The method of item 3653 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

3730. The method of item 3653 wherein the agent is a phosphatase inhibitor.

3731. The method of item 3653 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

3732. The method of item 3653 wherein the agent is a PKC inhibitor.

3733. The method of item 3653 wherein the agent is a platelet activating factor antagonist.

3734. The method of item 3653 wherein the agent is a platelet-derived growth factor receptor kinase inhibitor.

3735. The method of item 3653 wherein the agent is a prolyl hydroxylase inhibitor.

3736. The method of item 3653 wherein the agent is a polymorphonuclear neutrophil inhibitor.

3737. The method of item 3653 wherein the agent is a protein kinase B inhibitor.

3738. The method of item 3653 wherein the agent is a protein kinase C stimulant.

3739. The method of item 3653 wherein the agent is a purine nucleoside analogue.

3740. The method of item 3653 wherein the agent is a purinoreceptor P2X antagonist.

3741. The method of item 3653 wherein the agent is a Raf kinase inhibitor.

3742. The method of item 3653 wherein the agent is a reversible inhibitor of ErbB1 and ErbB2.

3743. The method of item 3653 wherein the agent is a ribonucleoside triphosphate reductase inhibitor.

3744. The method of item 3653 wherein the agent is an SDF-1 antagonist.

3745. The method of item 3653 wherein the agent is a sheddase inhibitor.

3746. The method of item 3653 wherein the agent is an SRC inhibitor.

3747. The method of item 3653 wherein the agent is a stromelysin inhibitor.

3748. The method of item 3653 wherein the agent is an Syk kinase inhibitor.

3749. The method of item 3653 wherein the agent is a telomerase inhibitor.

3750. The method of item 3653 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

3751. The method of item 3653 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

3752. The method of item 3653 wherein the agent is a Toll receptor inhibitor.

3753. The method of item 3653 wherein the agent is a tubulin antagonist.

3754. The method of item 3653 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-11 0 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

3755. The method of item 3653 wherein the agent is a VEGF inhibitor.

3756. The method of item 3653 wherein the agent is a vitamin D receptor agonist.

3757. The method of item 3653 wherein the agent is ZD-6474 (an angiogenesis inhibitor).

3758. The method of item 3653 wherein the agent is AP-23573 (an mTOR inhibitor).

3759. The method of item 3653 wherein the agent is synthadotin (a tubulin antagonist).

3760. The method of item 3653 wherein the agent is S-0885 (a collagenase inhibitor).

3761. The method of item 3653 wherein the agent is aplidine (an elongation factor-1 alpha inhibitor).

3762. The method of item 3653 wherein the agent is ixabepilone (an epithilone).

3763. The method of item 3653 wherein the agent is IDN-5390 (an angiogenesis inhibitor).

3764. The method of item 3653 wherein the agent is SB-2723005 (an angiogenesis inhibitor).

3765. The method of item 3653 wherein the agent is ABT-518 (an angiogenesis inhibitor).

3766. The method of item 3653 wherein the agent is combretastatin (an angiogenesis inhibitor).

3767. The method of item 3653 wherein the agent is anecortave acetate (an angiogenesis inhibitor).

3768. The method of item 3653 wherein the agent is SB-715992 (a kinesin antagonist).

3769. The method of item 3653 wherein the agent is temsirolimus (an mTOR inhibitor).

3770. The method of item 3653 wherein the agent is adalimumab (a TNFα antagonist).

3771. The method of item 3653, wherein the composition comprises a polymer.

3772. The method of item 3653, wherein the composition comprises a polymer, and the polymer is, or comprises, a copolymer.

3773. The method of item 3653, wherein the composition comprises a polymer, and the polymer is, or comprises, a block copolymer.

3774. The method of item 3653, wherein the composition comprises a polymer, and the polymer is, or comprises, a random copolymer.

3775. The method of item 3653, wherein the composition comprises a polymer, and the polymer is, or comprises, a biodegradable polymer.

3776. The method of item 3653, wherein the composition comprises a polymer, and the polymer is, or comprises, a non-biodegradable polymer.

3777. The method of item 3653, wherein the composition comprises a polymer, and the polymer is, or comprises, a hydrophilic polymer.

3778. The method of item 3653, wherein the composition comprises a polymer, and the polymer is, or comprises, a hydrophobic polymer.

3779. The method of item 3653, wherein the composition comprises a polymer, and the polymer is, or comprises, a polymer having hydrophilic domains.

3780. The method of item 3653, wherein the composition comprises a polymer, and the polymer is, or comprises, a polymer having hydrophobic domains.

3781. The method of item 3653, wherein the composition comprises a polymer, and the polymer is, or comprises, a non-conductive polymer.

3782. The method of item 3653, wherein the composition comprises a polymer, and the polymer is, or comprises, an elastomer.

3783. The method of item 3653, wherein the composition comprises a polymer, and the polymer is, or comprises, a hydrogel.

3784. The method of item 3653, wherein the composition comprises a polymer, and the polymer is, or comprises, a silicone polymer.

3785. The method of item 3653, wherein the composition comprises a polymer, and the polymer is, or comprises, a hydrocarbon polymer.

3786. The method of item 3653, wherein the composition comprises a polymer, and the polymer is, or comprises, a styrene-derived polymer.

3787. The method of item 3653, wherein the composition comprises a polymer, and the polymer is, or comprises, a butadiene-derived polymer.

3788. The method of item 3653, wherein the composition comprises a polymer, and the polymer is, or comprises, a macromer.

3789. The method of item 3653, wherein the composition comprises a polymer, and the polymer is, or comprises, a poly(ethylene glycol) polymer.

3790. The method of item 3653, wherein the composition comprises a polymer, and the polymer is, or comprises, an amorphous polymer.

3791. The method of item 3653, wherein the composition further comprises a second pharmaceutically active agent.

3792. The method of item 3653, wherein the composition further comprises an anti-inflammatory agent.

3793. The method of item 3653, wherein the composition further comprises an agent that inhibits infection.

3794. The method of item 3653, wherein the composition further comprises an anthracycline.

3795. The method of item 3653, wherein the composition further comprises doxorubicin.

3796. The method of item 3653 wherein the composition further comprises mitoxantrone.

3797. The method of item 3653 wherein the composition further comprises a fluoropyrimidine.

3798. The method of item 3653, wherein the composition further comprises 5-fluorouracil (5-FU).

3799. The method of item 3653, wherein the composition further comprises a folic acid antagonist.

3800. The method of item 3653, wherein the composition further comprises methotrexate.

3801. The method of item 3653, wherein the composition further comprises a podophylotoxin.

3802. The method of item 3653, wherein the composition further comprises etoposide.

3803. The method of item 3653, wherein the composition further comprises camptothecin.

3804. The method of item 3653, wherein the composition further comprises a hydroxyurea.

3805. The method of item 3653, wherein the composition further comprises a platinum complex.

3806. The method of item 3653, wherein the composition further comprises cisplatin.

3807. The method of item 3653 wherein the composition further comprises an anti-thrombotic agent.

3808. The method of item 3653, wherein the composition further comprises a visualization agent.

3809. The method of item 3653, wherein the composition further comprises a visualization agent, and the visualization agent is a radiopaque material, wherein the radiopaque material comprises a metal, a halogenated compound, or a barium containing compound.

3810. The method of item 3653, wherein the composition further comprises a visualization agent, and the visualization agent is, or comprises, barium, tantalum, or technetium.

3811. The method of item 3653, wherein the composition further comprises a visualization agent, and the visualization agent is, or comprises, an MRI responsive material.

3812. The method of item 3653, wherein the composition further comprises a visualization agent, and the visualization agent is, or comprises, a gadolinium chelate.

3813. The method of item 3653, wherein the composition further comprises a visualization agent, and the visualization agent is, or comprises, iron, magnesium, manganese, copper, or chromium.

3814. The method of item 3653, wherein the composition further comprises a visualization agent, and the visualization agent is, or comprises, iron oxide compound.

3815. The method of item 3653, wherein the composition further comprises a visualization agent, and the visualization agent is, or comprises, a dye, pigment, or colorant.

3816. The method of item 3653 wherein the agent is released in effective concentrations from the composition comprising the agent by diffusion over a period ranging from the time of administration to about 90 days.

3817. The method of item 3653 wherein the agent is released in effective concentrations from the composition comprising the agent by erosion of the composition over a period ranging from the time of administration to about 90 days.

3818. The method of item 3653 wherein the composition further comprises an inflammatory cytokine.

3819. The method of item 3653 wherein the composition further comprises an agent that stimulates cell proliferation.

3820. The method of item 3653 wherein the composition further comprises a polymeric carrier

3821. The method of item 3653 wherein the composition is in the form of a gel, paste, or spray.

3822. The method of item 3653 wherein the electrical device is partially constructed with the agent or the composition.

3823. The method of item 3653 wherein the electrical device is impregnated with the agent or the composition.

3824. The method of item 3653, wherein the agent or the composition forms a coating, and the coating directly contacts the electrical device.

3825. The method of item 3653, wherein the agent or the composition forms a coating, and the coating indirectly contacts the electrical device.

3826. The method of item 3653 wherein the agent or the composition forms a coating; and the coating partially covers the electrical device.

3827. The method of item 3653, wherein the agent or the composition forms a coating, and the coating completely covers the electrical device.

3828. The method of item 3653 wherein the agent or the composition is located within pores or holes of the electrical device.

3829. The method of item 3653 wherein the agent or the composition is located within a channel, lumen, or divet of the electrical device.

3830. The method of item 3653 wherein the electrical device further comprises an echogenic material.

3831. The method of item 3653 wherein the electrical device further comprises an echogenic material, wherein the echogenic material is in the form of a coating.

3832. The method of item 3653 wherein the electrical device is sterile.

3833. The method of item 3653 wherein the agent is delivered from the electrical device, wherein the agent is released into tissue in the vicinity of the electrical device after deployment of the electrical device.

3834. The method of item 3653 wherein the agent is delivered from the electrical device, wherein the agent is released into tissue in the vicinity of the electrical device after deployment of the electrical device, wherein the tissue is connective tissue.

3835. The method of item 3653 wherein the agent is delivered from the electrical device, wherein the agent is released into tissue in the vicinity of the electrical device after deployment of the electrical device, wherein the tissue is muscle tissue.

3836. The method of item 3653 wherein the agent is delivered from the electrical device, wherein the agent is released into tissue in the vicinity of the electrical device after deployment of the electrical device, wherein the tissue is nerve tissue.

3837. The method of item 3653 wherein the agent is delivered from the electrical device, wherein the agent is released into tissue in the vicinity of the electrical device after deployment of the electrical device, wherein the tissue is epithelium tissue.

3838. The method of item 3653 wherein the agent is delivered from the electrical device, wherein the agent is released in effective concentrations from the electrical device over a period ranging from the time of deployment of the electrical device to about 1 year.

3839. The method of item 3653 wherein the agent is delivered from the electrical device, wherein the agent is released in effective concentrations from the electrical device over a period ranging from about 1 month to 6 months.

3840. The method of item 3653 wherein the agent is delivered from the electrical device, wherein the agent is released in effective concentrations from the electrical device over a period ranging from about 1-90 days.

3841. The method of item 3653 wherein the agent is delivered from the electrical device, wherein the agent is released in effective concentrations from the electrical device at a constant rate.

3842. The method of item 3653 wherein the agent is delivered from the electrical device, wherein the agent is released in effective concentrations from the electrical device at an increasing rate.

3843. The method of item 3653 wherein the agent is delivered from the electrical device, wherein the agent is released in effective concentrations from the electrical device at a decreasing rate.

3844. The method of item 3653 wherein the agent is delivered from the electrical device, wherein the electrical device comprises about 0.01 μg to about 10 μg of the agent

3845. The method of item 3653 wherein the agent is delivered from the electrical device, wherein the electrical device comprises about 10 μg to about 10 mg of the agent.

3846. The method of item 3653 wherein the agent is delivered from the electrical device, wherein the electrical device comprises about 10 mg to about 250 mg of the agent.

3847. The method of item 3653 wherein the agent is delivered from the electrical device, wherein the electrical device comprises about 250 mg to about 1000 mg of the agent.

3848. The method of item 3653 wherein the agent is delivered from the electrical device, wherein the electrical device comprises about 1000 mg to about 2500 mg of the agent.

3849. The method of item 3653 wherein the agent is delivered from the electrical device, wherein a surface of the electrical device comprises less than 0.01 μg of the agent per mm² of electrical device surface to which the agent is applied.

3850. The method of item 3653 wherein the agent is delivered from the electrical device, wherein a surface of the electrical device comprises about 0.01 μg to about 1 μg of the agent per mm² of electrical device surface to which the agent is applied.

3851. The method of item 3653 wherein the agent is delivered from the electrical device, wherein a surface of the electrical device comprises about 1 μg to about 10 μg of the agent per mm² of electrical device surface to which the agent is applied.

3852. The method of item 3653 wherein the agent is delivered from the electrical device, wherein a surface of the electrical device comprises about 10 μg to about 250 μg of the agent per mm² of electrical device surface to which the agent is applied.

3853. The method of item 3653 wherein the agent is delivered from the electrical device, wherein a surface of the electrical device comprises about 250 μg to about 1000 μg of the agent per mm² of electrical device surface to which the agent is applied.

3854. The method of item 3653 wherein the agent is delivered from the electrical device, wherein a surface of the electrical device comprises about 1000 μg to about 2500 μg of the agent per mm² of electrical device surface to which the agent is applied.

3855. The method of item 3653, wherein the electrical device further comprises a coating, and the coating is a uniform coating.

3856. The method of item 3653, wherein the electrical device further comprises a coating, and the coating is a non-uniform coating.

3857. The method of item 3653, wherein the electrical device further comprises a coating, and the coating is a discontinuous coating.

3858. The method of item 3653, wherein the electrical device further comprises a coating, and the coating is a patterned coating.

3859. The method of item 3653, wherein the electrical device further comprises a coating, and the coating has a thickness of 100 μm or less.

3860. The method of item 3653, wherein the electrical device further comprises a coating, and the coating has a thickness of 10 μm or less.

3861. The method of item 3653, wherein the electrical device further comprises a coating, and the coating adheres to the surface of the electrical device upon deployment of the electrical device.

3862. The method of item 3653, wherein the electrical device further comprises a coating, and the coating is stable at room temperature for a period of at least 1 year.

3863. The method of item 3653, wherein the electrical device further comprises a coating, and the agent is present in the coating in an amount ranging between about 0.0001% to about 1% by weight.

3864. The method of item 3653, wherein the electrical device further comprises a coating, and the agent is present in the coating in an amount ranging between about 1% to about 10% by weight.

3865. The method of item 3653, wherein the electrical device further comprises a coating, and the agent is present in the coating in an amount ranging between about 10% to about 25% by weight.

3866. The method of item 3653, wherein the electrical device further comprises a coating, and the agent is present in the coating in an amount ranging between about 25% to about 70% by weight.

3867. The method of item 3653, wherein the electrical device further comprises a coating, and the coating comprises a polymer.

3868. The method of item 3653, wherein the electrical device comprises a first coating having a first composition and a second coating having a second composition.

3869. The method of item 3653, wherein the electrical device comprises a first coating having a first composition and a second coating having a second composition, wherein the first composition and the second composition are different.

3870. The method of item 3653 wherein the agent or the composition is affixed to the electrical device.

3871. The method of item 3653 wherein the agent or the composition is covalently attached to the electrical device.

3872. The method of item 3653 wherein the agent or the composition is non-covalently attached to the electrical device.

3873. The method of item 3653 wherein the electrical device comprises a coating that absorbs the agent or the composition.

3874. The method of item 3653 wherein the electrical device is interweaved with a thread composed of, or coated with, the agent or the composition.

3875. The method of item 3653 wherein a portion of the electrical device is covered with a sleeve that contains the agent or the composition.

3876. The method of item 3653 wherein the electrical device is completely covered with a sleeve that contains the agent or the composition.

3877. The method of item 3653 wherein a portion of the electrical device is covered with a mesh that contains the agent or the composition.

3878. The method of item 3653 wherein the electrical device is completely covered with a mesh that contains the agent or the composition.

3879. The method of item 3653 wherein the agent or the composition is applied to the electrical device surface prior to the placing of the electrical device into the host.

3880. The method of item 3653 wherein the agent or the composition is applied to the electrical device surface during the placing of the electrical device into the host.

3881. The method of item 3653 wherein the agent or the composition is applied to the electrical device surface immediately after the placing of the electrical device into the host.

3882. The method of item 3653 wherein the agent or the composition is applied to the surface of the tissue in the host surrounding the electrical device prior to the placing of the electrical device into the host.

3883. The method of item 3653 wherein the agent or the composition is applied to the surface of the tissue in the host surrounding the electrical device during the placing of the electrical device into the host.

3884. The method of item 3653 wherein the agent or the composition is applied to the surface of the tissue in the host surrounding the electrical device immediately after the placing of the electrical device into the host.

3885. The method of item 3653 wherein the agent or the composition is topically applied into the anatomical space where the electrical device is placed.

3886. The method of item 3653 wherein the agent or the composition is percutaneously injected into the tissue in the host surrounding the electrical device.

3887. The method for inhibiting scarring of any one of items 3653-3886 wherein the electrical device is a neurostimulator.

3888. The method for inhibiting scarring of any one of items 3653-3886 wherein the electrical device is a spinal cord stimulator.

3889. The method for inhibiting scarring of any one of items 3653-3886 wherein the electrical device is a brain stimulator.

3890. The method for inhibiting scarring of any one of items 3653-3886 wherein the electrical device is a vagus nerve stimulator.

3891. The method for inhibiting scarring of any one of items 3653-3886 wherein the electrical device is a sacral nerve stimulator.

3892. The method for inhibiting scarring of any one of items 3653-3886 wherein the electrical device is a gastric nerve stimulator.

3893. The method for inhibiting scarring of any one of items 3653-3886 wherein the electrical device is an auditory nerve stimulator.

3894. The method for inhibiting scarring of any one of items 3653-3886 wherein the electrical device delivers stimulation to organs.

3895. The method for inhibiting scarring of any one of items 3653-3886 wherein the electrical device delivers stimulation to bone.

3896. The method for inhibiting scarring of any one of items 3653-3886 wherein the electrical device delivers stimulation to muscles.

3897. The method for inhibiting scarring of any one of items 3653-3886 wherein the electrical device delivers stimulation to tissues.

3898. The method for inhibiting scarring of any one of items 3653-3886 wherein the electrical device is a device for continuous subarachnoid infusion.

3899. The method for inhibiting scarring of any one of items 3653-3886 wherein the electrical device is an implantable electrode.

3900. The method for inhibiting scarring of any one of items 3653-3886 wherein the electrical device is an implantable pulse generator.

3901. The method for inhibiting scarring of any one of items 3653-3886 wherein the electrical device is an electrical lead.

3902. The method for inhibiting scarring of any one of items 3653-3886 wherein the electrical device is a stimulation lead.

3903. The method for inhibiting scarring of any one of items 3653-3886 wherein the electrical device is a simulation catheter lead.

3904. The method for inhibiting scarring of any one of items 3653-3886 wherein the electrical device is cochlear implant.

3905. The method for inhibiting scarring of any one of items 3653-3886 wherein the electrical device is a microstimulator.

3906. The method for inhibiting scarring of any one of items 3653-3886 wherein the electrical device is battery powered.

3907. The method for inhibiting scarring of any one of items 3653-3886 wherein the electrical device is radio frequency powered.

3908. The method for inhibiting scarring of any one of items 3653-3886 wherein the electrical device is both battery and radio frequency powered.

3909. The method for inhibiting scarring of any one of items 3653-3886 wherein the electrical device is a cardiac rhythm management device.

3910. The method for inhibiting scarring of any one of items 3653-3886 wherein the electrical device is a cardiac pacemaker.

3911. The method for inhibiting scarring of any one of items 3653-3886 wherein the electrical device is an implantable cardioverter defibrillator system.

3912. The method for inhibiting scarring of any one of items 3653-3886 wherein the electrical device is a cardiac lead.

3913. The method for inhibiting scarring of any one of items 3653-3886 wherein the electrical device is a pacer lead.

3914. The method for inhibiting scarring of any one of items 3653-3886 wherein the electrical device is an endocardial lead.

3915. The method for inhibiting scarring of any one of items 3653-3886 wherein the electrical device is a cardioversion/defibrillator lead.

3916. The method for inhibiting scarring of any one of items 3653-3886 wherein the electrical device is an epicardial lead.

3917. The method for inhibiting scarring of any one of items 3653-3886 wherein the electrical device is an epicardial defibrillator lead.

3918. The method for inhibiting scarring of any one of items 3653-3886 wherein the electrical device is a patch defibrillator.

3919. The method for inhibiting scarring of any one of items 3653-3886 wherein the electrical device is a patch defibrillator lead.

3920. The method for inhibiting scarring of any one of items 3653-3886 wherein the electrical device is an electrical patch.

3921. The method for inhibiting scarring of any one of items 3653-3886 wherein the electrical device is a transvenous lead.

3922. The method for inhibiting scarring of any one of items 3653-3886 wherein the electrical device is an active fixation lead.

3923. The method for inhibiting scarring of any one of items 3653-3886 wherein the electrical device is a passive fixation lead.

3924. The method for inhibiting scarring of any one of items 3653-3886 wherein the electrical device is a sensing lead.

3925. The method for inhibiting scarring of any one of items 3653-3886 wherein the electrical device is a defibrillator.

3926. The method for inhibiting scarring of any one of items 3653-3886 wherein the electrical device is an implantable sensor.

3927. The method for inhibiting scarring of any one of items 3653-3886 wherein the electrical device is a left ventricular assist device.

3928. The method for inhibiting scarring of any one of items 3653-3886 wherein the electrical device is a pulse generator.

3929. The method for inhibiting scarring of any one of items 3653-3886 wherein the electrical device is a patch lead.

3930. The method for inhibiting scarring of any one of items 3653-3886 wherein the electrical device is an electrical patch.

3931. The method for inhibiting scarring of any one of items 3653-3886 wherein the electrical device is a cardiac stimulator.

3932. The method for inhibiting scarring of any one of items 3653-3886 wherein the electrical device is an electrical deviceable sensor.

3933. The method for inhibiting scarring of any one of items 3653-3886 wherein the electrical device is an electrical deviceable pump.

3934. The method for inhibiting scarring of any one of items 3653-3886 wherein the electrical device is a dural patch.

3935. The method for inhibiting scarring of any one of items 3653-3886 wherein the electrical device is a ventricular peritoneal shunt.

3936. The method for inhibiting scarring of any one of items 3653-3886 wherein the electrical device is a ventricular atrial shunt.

3937. The method for inhibiting scarring of any one of items 3653-3886 wherein the electrical device is adapted for treating or preventing epidural fibrosis post-laminectomy.

3938. The method for inhibiting scarring of any one of items 3653-3886 wherein the electrical device is adapted for treating or preventing cardiac rhythm abnormalities.

3939. The method for inhibiting scarring of any one of items 3653-3886 wherein the electrical device is adapted for treating or preventing atrial rhythm abnormalities.

3940. The method for inhibiting scarring of any one of items 3653-3886 wherein the electrical device is adapted for treating or preventing conduction abnormalities.

3941. The method for inhibiting scarring of any one of items 3653-3886 wherein the electrical device is adapted for treating or preventing ventricular rhythm abnormalities.

3942. The method for inhibiting scarring of any one of items 3653-3886 wherein the electrical device is adapted for treating or preventing pain.

3943. The method for inhibiting scarring of any one of items 3653-3886 wherein the electrical device is adapted for treating or preventing epilepsy.

3944. The method for inhibiting scarring of any one of items 3653-3886 wherein the electrical device is adapted for treating or preventing Parkinson's disease.

3945. The method for inhibiting scarring of any one of items 3653-3886 wherein the electrical device is adapted for treating or preventing movement disorders.

3946. The method for inhibiting scarring of any one of items 3653-3886 wherein the electrical device is adapted for treating or preventing obesity.

3947. The method for inhibiting scarring of any one of items 3653-3886 wherein the electrical device is adapted for treating or preventing depression.

3948. The method for inhibiting scarring of any one of items 3653-3886 wherein the electrical device is adapted for treating or preventing anxiety.

3949. The method for inhibiting scarring of any one of items 3653-3886 wherein the electrical device is adapted for treating or preventing hearing loss.

3950. The method for inhibiting scarring of any one of items 3653-3886 wherein the electrical device is adapted for treating or preventing ulcers.

3951. The method for inhibiting scarring of any one of items 3653-3886 wherein the electrical device is adapted for treating or preventing deep vein thrombosis.

3952. The method for inhibiting scarring of any one of items 3653-3886 wherein the electrical device is adapted for treating or preventing muscular atrophy.

3953. The method for inhibiting scarring of any one of items 3653-3886 wherein the electrical device is adapted for treating or preventing joint stiffness.

3954. The method for inhibiting scarring of any one of items 3653-3886 wherein the electrical device is adapted for treating or preventing muscle spasms.

3955. The method for inhibiting scarring of any one of items 3653-3886 wherein the electrical device is adapted for treating or preventing osteoporosis.

3956. The method for inhibiting scarring of any one of items 3653-3886 wherein the electrical device is adapted for treating or preventing scoliosis.

3957. The method for inhibiting scarring of any one of items 3653-3886 wherein the electrical device is adapted for treating or preventing spinal disc degeneration.

3958. The method for inhibiting scarring of any one of items 3653-3886 wherein the electrical device is adapted for treating or preventing spinal cord injury.

3959. The method for inhibiting scarring of any one of items 3653-3886 wherein the electrical device is adapted for treating or preventing urinary dysfunction.

3960. The method for inhibiting scarring of any one of items 3653-3886 wherein the electrical device is adapted for treating or preventing gastroparesis.

3961. The method for inhibiting scarring of any one of items 3653-3886 wherein the electrical device is adapted for treating or preventing malignancy.

3962. The method for inhibiting scarring of any one of items 3653-3886 wherein the electrical device is adapted for treating or preventing arachnoiditis.

3963. The method for inhibiting scarring of any one of items 3653-3886 wherein the electrical device is adapted for treating or preventing chronic disease.

3964. The method for inhibiting scarring of any one of items 3653-3886 wherein the electrical device is adapted for treating or preventing migraine.

3965. The method for inhibiting scarring of any one of items 3653-3886 wherein the electrical device is adapted for treating or preventing sleep disorders.

3966. The method for inhibiting scarring of any one of items 3653-3886 wherein the electrical device is adapted for treating or preventing dementia.

3967. The method for inhibiting scarring of any one of items 3653-3886 wherein the electrical device is adapted for treating or preventing Alzheimer's disease.

3968. A method for inhibiting scarring comprising placing a neurostimulator for treating chronic pain (i.e., an electrical device) and an anti-scarring agent or a composition comprising an ant-scarring agent into an animal host, wherein the agent inhibits scarring.

3969. The method of item 3968 wherein the agent is an adensosine A2A receptor antagonist.

3970. The method of item 3968 wherein the agent is an AKT inhibitor.

3971. The method of item 3968 wherein the agent is an alpha 2 integrin antagonist, wherein the alpha 2 integrin antagonist is Pharmaprojects No. 5754 (Merck KgaA).

3972. The method of item 3968 wherein the agent is an alpha 4 integrin antagonist.

3973. The method of item 3968 wherein the agent is an alpha 7 nicotinic receptor agonist.

3974. The method of item 3968 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

3975. The method of item 3968 wherein the agent is an apoptosis antagonist.

3976. The method of item 3968 wherein the agent is an apoptosis activator.

3977. The method of item 3968 wherein the agent is a beta 1 integrin antagonist.

3978. The method of item 3968 wherein the agent is a beta tubulin inhibitor.

3979. The method of item 3968 wherein the agent is a blocker of enzyme production in Hepatitis C.

3980. The method of item 3968 wherein the agent is a Bruton's tyrosine kinase inhibitor.

3981. The method of item 3968 wherein the agent is a calcineurin inhibitor.

3982. The method of item 3968 wherein the agent is a caspase 3 inhibitor.

3983. The method of item 3968 wherein the agent is a CC chemokine receptor antagonist.

3984. The method of item 3968 wherein the agent is a cell cycle inhibitor selected from the group consisting of SNS-595 (Sunesis), synthadotin, KRX-0403, and an analogue or derivative thereof.

3985. The method of item 3968 wherein the agent is a cathepsin B inhibitor.

3986. The method of item 3968 wherein the agent is a cathepsin K inhibitor, wherein the cathepsin K inhibitor is 462795 (GlaxoSmithKline), INPL-022-D6 (Amura Therapeutics), or an analogue or derivative thereof.

3987. The method of item 3968 wherein the agent is a cathepsin L inhibitor.

3988. The method of item 3968 wherein the agent is a CD40 antagonist.

3989. The method of item 3968 wherein the agent is a chemokine receptor agonist.

3990. The method of item 3968 wherein the agent is a chymase inhibitor.

3991. The method of item 3968 wherein the agent is a collagenase antagonist.

3992. The method of item 3968 wherein the agent is a CXCR antagonist.

3993. The method of item 3968 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

3994. The method of item 3968 wherein the agent is a cyclooxygenase 1 inhibitor.

3995. The method of item 3968 wherein the agent is a DHFR inhibitor.

3996. The method of item 3968 wherein the agent is a dual integrin inhibitor.

3997. The method of item 3968 wherein the agent is an elastase inhibitor.

3998. The method of item 3968 wherein the agent is an elongation factor-1 alpha inhibitor.

3999. The method of item 3968 wherein the agent is an endothelial growth factor antagonist.

4000. The method of item 3968 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

4001. The method of item 3968 wherein the agent is an endotoxin antagonist.

4002. The method of item 3968 wherein the agent is an epothilone and tubulin binder.

4003. The method of item 3968 wherein the agent is an estrogen receptor antagonist.

4004. The method of item 3968 wherein the agent is an FGF inhibitor.

4005. The method of item 3968 wherein the agent is a farnexyl transferase inhibitor.

4006. The method of item 3968 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

4007. The method of item 3968 wherein the agent is an FLT-3 kinase inhibitor.

4008. The method of item 3968 wherein the agent is an FGF receptor kinase inhibitor.

4009. The method of item 3968 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

4010. The method of item 3968 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

4011. The method of item 3968 wherein the agent is a histone deacetylase inhibitor.

4012. The method of item 3968 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

4013. The method of item 3968 wherein the agent is an ICAM inhibitor.

4014. The method of item 3968 wherein the agent is an IL, ICE and IRAK antagonist, wherein the antagonist is a CJ-14877, CP-424174 (Pfizer), NF-61 (Negma-Lerads), and an analogue or derivative thereof.

4015. The method of item 3968 wherein the agent is an IL-2 inhibitor.

4016. The method of item 3968 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof

4017. The method of item 3968 wherein the agent is an IMPDH (inosine monophosphate).

4018. The method of item 3968 wherein the agent is an integrin antagonist.

4019. The method of item 3968 wherein the agent is an interleukin antagonist.

4020. The method of item 3968 wherein the agent is an inhibitor of type III receptor tyrosine kinase.

4021. The method of item 3968 wherein the agent is an irreversible inhibitor of enzyme methionine aminopeptidase type 2.

4022. The method of item 3968 wherein the agent is an isozyme selective delta protein kinase C inhibitor.

4023. The method of item 3968 wherein the agent a JAK3 enzyme inhibitor.

4024. The method of item 3968 wherein the agent is a JNK inhibitor.

4025. The method of item 3968 wherein the agent is a kinase inhibitor.

4026. The method of item 3968 wherein the agent is kinesin antagonist.

4027. The method of item 3968 wherein the agent is a kinesin antagonist.

4028. The method of item 3968 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

4029. The method of item 3968 wherein the agent is an MAP kinase inhibitor.

4030. The method of item 3968 wherein the agent is a matrix metalloproteinase inhibitor.

4031. The method of item 3968 wherein the agent is an MCP-CCR2 inhibitor.

4032. The method of item 3968 wherein the agent is an mTOR inhibitor.

4033. The method of item 3968 wherein the agent is an mTOR kinase inhibitor.

4034. The method of item 3968 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

4035. The method of item 3968 wherein the agent is an MIF inhibitor.

4036. The method of item 3968 wherein the agent is an MMP inhibitor.

4037. The method of item 3968 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

4038. The method of item 3968 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

4039. The method of item 3968 wherein the agent is a nitric oxide agonist.

4040. The method of item 3968 wherein the agent is an ornithine decarboxylase inhibitor.

4041. The method of item 3968 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

4042. The method of item 3968 wherein the agent is a palmitoyl-protein thioesterase inhibitor.

4043. The method of item 3968 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

4044. The method of item 3968 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

4045. The method of item 3968 wherein the agent is a phosphatase inhibitor.

4046. The method of item 3968 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

4047. The method of item 3968 wherein the agent is a PKC inhibitor.

4048. The method of item 3968 wherein the agent is a platelet activating factor antagonist.

4049. The method of item 3968 wherein the agent is a platelet-derived growth factor receptor kinase inhibitor.

4050. The method of item 3968 wherein the agent is a prolyl hydroxylase inhibitor.

4051. The method of item 3968 wherein the agent is a polymorphonuclear neutrophil inhibitor.

4052. The method of item 3968 wherein the agent is a protein kinase B inhibitor.

4053. The method of item 3968 wherein the agent is a protein kinase C stimulant.

4054. The method of item 3968 wherein the agent is a purine nucleoside analogue.

4055. The method of item 3968 wherein the agent is a purinoreceptor P2X antagonist.

4056. The method of item 3968 wherein the agent is a Raf kinase inhibitor.

4057. The method of item 3968 wherein the agent is a reversible inhibitor of ErbB1 and ErbB2.

4058. The method of item 3968 wherein the agent is a ribonucleoside triphosphate reductase inhibitor.

4059. The method of item 3968 wherein the agent is an SDF-1 antagonist.

4060. The method of item 3968 wherein the agent is a sheddase inhibitor.

4061. The method of item 3968 wherein the agent is an SRC inhibitor.

4062. The method of item 3968 wherein the agent is a stromelysin inhibitor.

4063. The method of item 3968 wherein the agent is an Syk kinase inhibitor.

4064. The method of item 3968 wherein the agent is a telomerase inhibitor.

4065. The method of item 3968 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

4066. The method of item 3968 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

4067. The method of item 3968 wherein the agent is a Toll receptor inhibitor.

4068. The method of item 3968 wherein the agent is a tubulin antagonist.

4069. The method of item 3968 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-11 0 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

4070. The method of item 3968 wherein the agent is a VEGF inhibitor.

4071. The method of item 3968 wherein the agent is a vitamin D receptor agonist.

4072. The method of item 3968 wherein the agent is ZD-6474 (an angiogenesis inhibitor).

4073. The method of item 3968 wherein the agent is AP-23573 (an mTOR inhibitor).

4074. The method of item 3968 wherein the agent is synthadotin (a tubulin antagonist).

4075. The method of item 3968 wherein the agent is S-0885 (a collagenase inhibitor).

4076. The method of item 3968 wherein the agent is aplidine (an elongation factor-1 alpha inhibitor).

4077. The method of item 3968 wherein the agent is ixabepilone (an epithilone).

4078. The method of item 3968 wherein the agent is IDN-5390 (an angiogenesis inhibitor).

4079. The method of item 3968 wherein the agent is SB-2723005 (an angiogenesis inhibitor).

4080. The method of item 3968 wherein the agent is ABT-518 (an angiogenesis inhibitor).

4081. The method of item 3968 wherein the agent is combretastatin (an angiogenesis inhibitor).

4082. The method of item 3968 wherein the agent is anecortave acetate (an angiogenesis inhibitor).

4083. The method of item 3968 wherein the agent is SB-715992 (a kinesin antagonist).

4084. The method of item 3968 wherein the agent is temsirolimus (an mTOR inhibitor).

4085. The method of item 3968 wherein the agent is adalimumab (a TNFα antagonist).

4086. The method of item 3968, wherein the composition comprises a polymer.

4087. The method of item 3968, wherein the composition comprises a polymer, and the polymer is, or comprises, a copolymer.

4088. The method of item 3968, wherein the composition comprises a polymer, and the polymer is, or comprises, a block copolymer.

4089. The method of item 3968, wherein the composition comprises a polymer, and the polymer is, or comprises, a random copolymer.

4090. The method of item 3968, wherein the composition comprises a polymer, and the polymer is, or comprises, a biodegradable polymer.

4091. The method of item 3968, wherein the composition comprises a polymer, and the polymer is, or comprises, a non-biodegradable polymer.

4092. The method of item 3968, wherein the composition comprises a polymer, and the polymer is, or comprises, a hydrophilic polymer.

4093. The method of item 3968, wherein the composition comprises a polymer, and the polymer is, or comprises, a hydrophobic polymer.

4094. The method of item 3968, wherein the composition comprises a polymer, and the polymer is, or comprises, a polymer having hydrophilic domains.

4095. The method of item 3968, wherein the composition comprises a polymer, and the polymer is, or comprises, a polymer having hydrophobic domains.

4096. The method of item 3968, wherein the composition comprises a polymer, and the polymer is, or comprises, a non-conductive polymer.

4097. The method of item 3968, wherein the composition comprises a polymer, and the polymer is, or comprises, an elastomer.

4098. The method of item 3968, wherein the composition comprises a polymer, and the polymer is, or comprises, a hydrogel.

4099. The method of item 3968, wherein the composition comprises a polymer, and the polymer is, or comprises, a silicone polymer.

4100. The method of item 3968, wherein the composition comprises a polymer, and the polymer is, or comprises, a hydrocarbon polymer.

4101. The method of item 3968, wherein the composition comprises a polymer, and the polymer is, or comprises, a styrene-derived polymer.

4102. The method of item 3968, wherein the composition comprises a polymer, and the polymer is, or comprises, a butadiene-derived polymer.

4103. The method of item 3968, wherein the composition comprises a polymer, and the polymer is, or comprises, a macromer.

4104. The method of item 3968, wherein the composition comprises a polymer, and the polymer is, or comprises, a poly(ethylene glycol) polymer.

4105. The method of item 3968, wherein the composition comprises a polymer, and the polymer is, or comprises, an amorphous polymer.

4106. The method of item 3968, wherein the composition further comprises a second pharmaceutically active agent.

4107. The method of item 3968, wherein the composition further comprises an anti-inflammatory agent.

4108. The method of item 3968, wherein the composition further comprises an agent that inhibits infection.

4109. The method of item 3968, wherein the composition further comprises an anthracycline.

4110. The method of item 3968, wherein the composition further comprises doxorubicin.

4111. The method of item 3968 wherein the composition further comprises mitoxantrone.

4112. The method of item 3968 wherein the composition further comprises a fluoropyrimidine.

4113. The method of item 3968, wherein the composition further comprises 5-fluorouracil (5-FU).

4114. The method of item 3968, wherein the composition further comprises a folic acid antagonist.

4115. The method of item 3968, wherein the composition further comprises methotrexate.

4116. The method of item 3968, wherein the composition further comprises a podophylotoxin.

4117. The method of item 3968, wherein the composition further comprises etoposide.

4118. The method of item 3968, wherein the composition further comprises camptothecin.

4119. The method of item 3968, wherein the composition further comprises a hydroxyurea.

4120. The method of item 3968, wherein the composition further comprises a platinum complex.

4121. The method of item 3968, wherein the composition further comprises cisplatin.

4122. The method of item 3968 wherein the composition further comprises an anti-thrombotic agent.

4123. The method of item 3968, wherein the composition further comprises a visualization agent.

4124. The method of item 3968, wherein the composition further comprises a visualization agent, and the visualization agent is a radiopaque material, wherein the radiopaque material comprises a metal, a halogenated compound, or a barium containing compound.

4125. The method of item 3968, wherein the composition further comprises a visualization agent, and the visualization agent is, or comprises, barium, tantalum, or technetium.

4126. The method of item 3968, wherein the composition further comprises a visualization agent, and the visualization agent is, or comprises, an MRI responsive material

4127. The method of item 3968, wherein the composition further comprises a visualization agent, and the visualization agent is, or comprises, a gadolinium chelate.

4128. The method of item 3968, wherein the composition further comprises a visualization agent, and the visualization agent is, or comprises, iron, magnesium, manganese, copper, or chromium.

4129. The method of item 3968, wherein the composition further comprises a visualization agent, and the visualization agent is, or comprises, iron oxide compound.

4130. The method of item 3968, wherein the composition further comprises a visualization agent, and the visualization agent is, or comprises, a dye, pigment, or colorant.

4131. The method of item 3968 wherein the agent is released in effective concentrations from the composition comprising the agent by diffusion over a period ranging from the time of administration to about 90 days.

4132. The method of item 3968 wherein the agent is released in effective concentrations from the composition comprising the agent by erosion of the composition over a period ranging from the time of administration to about 90 days.

4133. The method of item 3968 wherein the composition further comprises an inflammatory cytokine.

4134. The method of item 3968 wherein the composition further comprises an agent that stimulates cell proliferation.

4135. The method of item 3968 wherein the composition further comprises a polymeric carrier.

4136. The method of item 3968 wherein the composition is in the form of a gel, paste, or spray.

4137. The method of item 3968 wherein the electrical device is partially constructed with the agent or the composition.

4138. The method of item 3968 wherein the electrical device is impregnated with the agent or the composition.

4139. The method of item 3968, wherein the agent or the composition forms a coating, and the coating directly contacts the electrical device.

4140. The method of item 3968, wherein the agent or the composition forms a coating, and the coating indirectly contacts the electrical device.

4141. The method of item 3968 wherein the agent or the composition forms a coating, and the coating partially covers the electrical device.

4142. The method of item 3968, wherein the agent or the composition forms a coating, and the coating completely covers the electrical device.

4143. The method of item 3968 wherein the agent or the composition is located within pores or holes of the electrical device.

4144. The method of item 3968 wherein the agent or the composition is located within a channel, lumen, or divet of the electrical device.

4145. The method of item 3968 wherein the electrical device further comprises an echogenic material.

4146. The method of item 3968 wherein the electrical device further comprises an echogenic material, wherein the echogenic material is in the form of a coating.

4147. The method of item 3968 wherein the electrical device is sterile.

4148. The method of item 3968 wherein the agent is delivered from the electrical device, wherein the agent is released into tissue in the vicinity of the electrical device after deployment of the electrical device.

4149. The method of item 3968 wherein the agent is delivered from the electrical device, wherein the agent is released into tissue in the vicinity of the electrical device after deployment of the electrical device, wherein the tissue is connective tissue.

4150. The method of item 3968 wherein the agent is delivered from the electrical device, wherein the agent is released into tissue in the vicinity of the electrical device after deployment of the electrical device, wherein the tissue is muscle tissue.

4151. The method of item 3968 wherein the agent is delivered from the electrical device, wherein the agent is released into tissue in the vicinity of the electrical device after deployment of the electrical device, wherein the tissue is nerve tissue.

4152. The method of item 3968 wherein the agent is delivered from the electrical device, wherein the agent is released into tissue in the vicinity of the electrical device after deployment of the electrical device, wherein the tissue is epithelium tissue.

4153. The method of item 3968 wherein the agent is delivered from the electrical device, wherein the agent is released in effective concentrations from the electrical device over a period ranging from the time of deployment of the electrical device to about 1 year.

4154. The method of item 3968 wherein the agent is delivered from the electrical device, wherein the agent is released in effective concentrations from the electrical device over a period ranging from about 1 month to 6 months.

4155. The method of item 3968 wherein the agent is delivered from the electrical device, wherein the agent is released in effective concentrations from the electrical device over a period ranging from about 1-90 days.

4156. The method of item 3968 wherein the agent is delivered from the electrical device, wherein the agent is released in effective concentrations from the electrical device at a constant rate.

4157. The method of item 3968 wherein the agent is delivered from the electrical device, wherein the agent is released in effective concentrations from the electrical device at an increasing rate.

4158. The method of item 3968 wherein the agent is delivered from the electrical device, wherein the agent is released in effective concentrations from the electrical device at a decreasing rate.

4159. The method of item 3968 wherein the agent is delivered from the electrical device, wherein the electrical device comprises about 0.01 μg to about 10 μg of the agent.

4160. The method of item 3968 wherein the agent is delivered from the electrical device, wherein the electrical device comprises about 10 μg to about 10 mg of the agent.

4161. The method of item 3968 wherein the agent is delivered from the electrical device, wherein the electrical device comprises about 10 mg to about 250 mg of the agent.

4162. The method of item 3968 wherein the agent is delivered from the electrical device, wherein the electrical device comprises about 250 mg to about 1000 mg of the agent.

4163. The method of item 3968 wherein the agent is delivered from the electrical device, wherein the electrical device comprises about 1000 mg to about 2500 mg of the agent.

4164. The method of item 3968 wherein the agent is delivered from the electrical device, wherein a surface of the electrical device comprises less than 0.01 μg of the agent per mm² of electrical device surface to which the agent is applied.

4165. The method of item 3968 wherein the agent is delivered from the electrical device, wherein a surface of the electrical device comprises about 0.01 μg to about 1 μg of the agent per mm² of electrical device surface to which the agent is applied.

4166. The method of item 3968 wherein the agent is delivered from the electrical device, wherein a surface of the electrical device comprises about 1 μg to about 10 μg of the agent per mm² of electrical device surface to which the agent is applied.

4167. The method of item 3968 wherein the agent is delivered from the electrical device, wherein a surface of the electrical device comprises about 10 μg to about 250 μg of the agent per mm² of electrical device surface to which the agent is applied.

4168. The method of item 3968 wherein the agent is delivered from the electrical device, wherein a surface of the electrical device comprises about 250 μg to about 1000 μg of the agent per mm² of electrical device surface to which the agent is applied.

4169. The method of item 3968 wherein the agent is delivered from the electrical device, wherein a surface of the electrical device comprises about 1000 μg to about 2500 μg of the agent per mm² of electrical device surface to which the agent is applied.

4170. The method of item 3968, wherein the electrical device further comprises a coating, and the coating is a uniform coating.

4171. The method of item 3968, wherein the electrical device further comprises a coating, and the coating is a non-uniform coating.

4172. The method of item 3968, wherein the electrical device further comprises a coating, and the coating is a discontinuous coating.

4173. The method of item 3968, wherein the electrical device further comprises a coating, and the coating is a patterned coating.

4174. The method of item 3968, wherein the electrical device further comprises a coating, and the coating has a thickness of 100 μm or less.

4175. The method of item 3968, wherein the electrical device further comprises a coating, and the coating has a thickness of 10 μm or less.

4176. The method of item 3968, wherein the electrical device further comprises a coating, and the coating adheres to the surface of the electrical device upon deployment of the electrical device.

4177. The method of item 3968, wherein the electrical device further comprises a coating, and the coating is stable at room temperature for a period of at least 1 year.

4178. The method of item 3968, wherein the electrical device further comprises a coating, and the agent is present in the coating in an amount ranging between about 0.0001% to about 1% by weight.

4179. The method of item 3968, wherein the electrical device further comprises a coating, and the agent is present in the coating in an amount ranging between about 1% to about 10% by weight.

4180. The method of item 3968, wherein the electrical device further comprises a coating, and the agent is present in the coating in an amount ranging between about 10% to about 25% by weight.

4181. The method of item 3968, wherein the electrical device further comprises a coating, and the agent is present in the coating in an amount ranging between about 25% to about 70% by weight.

4182. The method of item 3968, wherein the electrical device further comprises a coating, and the coating comprises a polymer.

4183. The method of item 3968, wherein the electrical device comprises a first coating having a first composition and a second coating having a second composition.

4184. The method of item 3968, wherein the electrical device comprises a first coating having a first composition and a second coating having a second composition, wherein the first composition and the second composition are different.

4185. The method of item 3968 wherein the agent or the composition is affixed to the electrical device.

4186. The method of item 3968 wherein the agent or the composition is covalently attached to the electrical device.

4187. The method of item 3968 wherein the agent or the composition is non-covalently attached to the electrical device.

4188. The method of item 3968 wherein the electrical device comprises a coating that absorbs the agent or the composition.

4189. The method of item 3968 wherein the electrical device is interweaved with a thread composed of, or coated with, the agent or the composition.

4190. The method of item 3968 wherein a portion of the electrical device is covered with a sleeve that contains the agent or the composition.

4191. The method of item 3968 wherein the electrical device is completely covered with a sleeve that contains the agent or the composition.

4192. The method of item 3968 wherein a portion of the electrical device is covered with a mesh that contains the agent or the composition.

4193. The method of item 3968 wherein the electrical device is completely covered with a mesh that contains the agent or the composition.

4194. The method of item 3968 wherein the agent or the composition is applied to the electrical device surface prior to the placing of the electrical device into the host.

4195. The method of item 3968 wherein the agent or the composition is applied to the electrical device surface during the placing of the electrical device into the host.

4196. The method of item 3968 wherein the agent or the composition is applied to the electrical device surface immediately after the placing of the electrical device into the host.

4197. The method of item 3968 wherein the agent or the composition is applied to the surface of the tissue in the host surrounding the electrical device prior to the placing of the electrical device into the host.

4198. The method of item 3968 wherein the agent or the composition is applied to the surface of the tissue in the host surrounding the electrical device during the placing of the electrical device into the host

4199. The method of item 3968 wherein the agent or the composition is applied to the surface of the tissue in the host surrounding the electrical device immediately after the placing of the electrical device into the host

4200. The method of item 3968 wherein the agent or the composition is topically applied into the anatomical space where the electrical device is placed.

4201. The method of item 3968 wherein the agent or the composition is percutaneously injected into the tissue in the host surrounding the electrical device.

4202. The method for inhibiting scarring of items 3968-4201 wherein the chronic pain results from injury.

4203. The method for inhibiting scarring of items 3968-4201 wherein the chronic pain results from an illness.

4204. The method for inhibiting scarring of items 3968-4201 wherein the chronic pain results from scoliosis.

4205. The method for inhibiting scarring of items 3968-4201 wherein the chronic pain results from spinal disc degeneration.

4206. The method for inhibiting scarring of items 3968-4201 wherein the chronic pain results from malignancy.

4207. The method for inhibiting scarring of items 3968-4201 wherein the chronic pain results from arachnoiditis.

4208. The method for inhibiting scarring of items 3968-4201 wherein the chronic pain results from a chronic disease.

4209. The method for inhibiting scarring of items 3968-4201 wherein the chronic pain results from a pain syndrome.

4210. The method for inhibiting scarring of items 3968-4201 wherein the neurostimulator comprises a lead that delivers electrical stimulation to a nerve and an electrical connection that connects a power source to the lead.

4211. The method for inhibiting scarring of items 3968-4201 wherein the neurostimulator is adapted for spinal cord stimulation, and comprises a sensor that detects the position of the spine and a stimulator that emits pulses that decrease in amplitude when the back is in a supine position.

4212. The method for inhibiting scarring of items 3968-4201 wherein the neurostimulator comprises an electrode and a control circuit that generates pulses and rest period based on intervals corresponding to the host body's activity and regeneration period.

4213. The method for inhibiting scarring of items 3968-4201 wherein the neurostimulator comprises a stimulation catheter lead and an electrode.

4214. The method for inhibiting scarring of items 3968-4201 wherein the neurostimulator is a self-centering epidural spinal cord lead.

4215. A method for inhibiting scarring comprising placing a neurostimulator for treating Parkinson's Disease (i.e., an electrical device) and an anti-scarring agent or a composition comprising an ant-scarring agent into an animal host, wherein the agent inhibits scarring.

4216. The method of item 4215 wherein the agent is an adensosine A2A receptor antagonist.

4217. The method of item 4215 wherein the agent is an AKT inhibitor.

4218. The method of item 4215 wherein the agent is an alpha 2 integrin antagonist, wherein the alpha 2 integrin antagonist is Pharmaprojects No. 5754 (Merck KgaA).

4219. The method of item 4215 wherein the agent is an alpha 4 integrin antagonist.

4220. The method of item 4215 wherein the agent is an alpha 7 nicotinic receptor agonist.

4221. The method of item 4215 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

4222. The method of item 4215 wherein the agent is an apoptosis antagonist.

4223. The method of item 4215 wherein the agent is an apoptosis activator.

4224. The method of item 4215 wherein the agent is a beta 1 integrin antagonist.

4225. The method of item 4215 wherein the agent is a beta tubulin inhibitor.

4226. The method of item 4215 wherein the agent is a blocker of enzyme production in Hepatitis C.

4227. The method of item 4215 wherein the agent is a Bruton's tyrosine kinase inhibitor.

4228. The method of item 4215 wherein the agent is a calcineurin inhibitor.

4229. The method of item 4215 wherein the agent is a caspase 3 inhibitor.

4230. The method of item 4215 wherein the agent is a CC chemokine receptor antagonist.

4231. The method of item 4215 wherein the agent is a cell cycle inhibitor selected from the group consisting of SNS-595 (Sunesis), synthadotin, KRX-0403, and an analogue or derivative thereof.

4232. The method of item 4215 wherein the agent is a cathepsin B inhibitor.

4233. The method of item 4215 wherein the agent is a cathepsin K inhibitor, wherein the cathepsin K inhibitor is 462795 (GlaxoSmithKline), INPL-022-D6 (Amura Therapeutics), or an analogue or derivative thereof.

4234. The method of item 4215 wherein the agent is a cathepsin L inhibitor.

4235. The method of item 4215 wherein the agent is a CD40 antagonist.

4236. The method of item 4215 wherein the agent is a chemokine receptor agonist.

4237. The method of item 4215 wherein the agent is a chymase inhibitor.

4238. The method of item 4215 wherein the agent is a collagenase antagonist.

4239. The method of item 4215 wherein the agent is a CXCR antagonist.

4240. The method of item 4215 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

4241. The method of item 4215 wherein the agent is a cyclooxygenase 1 inhibitor.

4242. The method of item 4215 wherein the agent is a DHFR inhibitor.

4243. The method of item 4215 wherein the agent is a dual integrin inhibitor.

4244. The method of item 4215 wherein the agent is an elastase inhibitor.

4245. The method of item 4215 wherein the agent is an elongation factor-1 alpha inhibitor.

4246. The method of item 4215 wherein the agent is an endothelial growth factor antagonist.

4247. The method of item 4215 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

4248. The method of item 4215 wherein the agent is an endotoxin antagonist.

4249. The method of item 4215 wherein the agent is an epothilone and tubulin binder.

4250. The method of item 4215 wherein the agent is an estrogen receptor antagonist.

4251. The method of item 4215 wherein the agent is an FGF inhibitor.

4252. The method of item 4215 wherein the agent is a farnexyl transferase inhibitor.

4253. The method of item 4215 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

4254. The method of item 4215 wherein the agent is an FLT-3 kinase inhibitor.

4255. The method of item 4215 wherein the agent is an FGF receptor kinase inhibitor.

4256. The method of item 4215 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

4257. The method of item 4215 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

4258. The method of item 4215 wherein the agent is a histone deacetylase inhibitor.

4259. The method of item 4215 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

4260. The method of item 4215 wherein the agent is an ICAM inhibitor.

4261. The method of item 4215 wherein the agent is an IL, ICE and IRAK antagonist, wherein the antagonist is a CJ-14877, CP-424174 (Pfizer), NF-61 (Negma-Lerads), and an analogue or derivative thereof.

4262. The method of item 4215 wherein the agent is an IL-2 inhibitor.

4263. The method of item 4215 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

4264. The method of item 4215 wherein the agent is an IMPDH (inosine monophosphate).

4265. The method of item 4215 wherein the agent is an integrin antagonist.

4266. The method of item 4215 wherein the agent is an interleukin antagonist.

4267. The method of item 4215 wherein the agent is an inhibitor of type III receptor tyrosine kinase.

4268. The method of item 4215 wherein the agent is an irreversible inhibitor of enzyme methionine aminopeptidase type 2.

4269. The method of item 4215 wherein the agent is an isozyme selective delta protein kinase C inhibitor.

4270. The method of item 4215 wherein the agent a JAK3 enzyme inhibitor.

4271. The method of item 4215 wherein the agent is a JNK inhibitor.

4272. The method of item 4215 wherein the agent is a kinase inhibitor.

4273. The method of item 4215 wherein the agent is kinesin antagonist.

4274. The method of item 4215 wherein the agent is a kinesin antagonist.

4275. The method of item 4215 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

4276. The method of item 4215 wherein the agent is an MAP kinase inhibitor.

4277. The method of item 4215 wherein the agent is a matrix metalloproteinase inhibitor.

4278. The method of item 4215 wherein the agent is an MCP-CCR2 inhibitor.

4279. The method of item 4215 wherein the agent is an mTOR inhibitor.

4280. The method of item 4215 wherein the agent is an mTOR kinase inhibitor.

4281. The method of item 4215 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

4282. The method of item 4215 wherein the agent is an MIF inhibitor.

4283. The method of item 4215 wherein the agent is an MMP inhibitor.

4284. The method of item 4215 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

4285. The method of item 4215 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

4286. The method of item 4215 wherein the agent is a nitric oxide agonist.

4287. The method of item 4215 wherein the agent is an ornithine decarboxylase inhibitor.

4288. The method of item 4215 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

4289. The method of item 4215 wherein the agent is a palmitoyl-protein thioesterase inhibitor.

4290. The method of item 4215 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No 387867-13-2) (Millennium Pharmaceuticals), vatalanib (Novartis), ZK-CDK (Schering AG), and an analogue or derivative thereof

4291. The method of item 4215 wherein the agent is (−)-halofenate (Metabolex), AMG-131 (Amgen), antidiabetics from Japan Tobacco, AZD-4619, AZD-8450, AZD-8677 (AstraZeneca), DRF-1 0945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

4292. The method of item 4215 wherein the agent is a phosphatase inhibitor.

4293. The method of item 4215 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

4294. The method of item 4215 wherein the agent is a PKC inhibitor.

4295. The method of item 4215 wherein the agent is a platelet activating factor antagonist.

4296. The method of item 4215 wherein the agent is a platelet-derived growth factor receptor kinase inhibitor.

4297. The method of item 4215 wherein the agent is a prolyl hydroxylase inhibitor.

4298. The method of item 4215 wherein the agent is a polymorphonuclear neutrophil inhibitor.

4299. The method of item 4215 wherein the agent is a protein kinase B inhibitor.

4300. The method of item 4215 wherein the agent is a protein kinase C stimulant.

4301. The method of item 4215 wherein the agent is a purine nucleoside analogue.

4302. The method of item 4215 wherein the agent is a purinoreceptor P2X antagonist.

4303. The method of item 4215 wherein the agent is a Raf kinase inhibitor.

4304. The method of item 4215 wherein the agent is a reversible inhibitor of ErbB1 and ErbB2.

4305. The method of item 4215 wherein the agent is a ribonucleoside triphosphate reductase inhibitor.

4306. The method of item 4215 wherein the agent is an SDF-1 antagonist.

4307. The method of item 4215 wherein the agent is a sheddase inhibitor.

4308. The method of item 4215 wherein the agent is an SRC inhibitor.

4309. The method of item 4215 wherein the agent is a stromelysin inhibitor.

4310. The method of item 4215 wherein the agent is an Syk kinase inhibitor.

4311. The method of item 4215 wherein the agent is a telomerase inhibitor.

4312. The method of item 4215 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

4313. The method of item 4215 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

4314. The method of item 4215 wherein the agent is a Toll receptor inhibitor.

4315. The method of item 4215 wherein the agent is a tubulin antagonist.

4316. The method of item 4215 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HE R-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

4317. The method of item 4215 wherein the agent is a VEGF inhibitor.

4318. The method of item 4215 wherein the agent is a vitamin D receptor agonist.

4319. The method of item 4215 wherein the agent is ZD-6474 (an angiogenesis inhibitor).

4320. The method of item 4215 wherein the agent is AP-23573 (an mTOR inhibitor).

4321. The method of item 4215 wherein the agent is synthadotin (a tubulin antagonist).

4322. The method of item 4215 wherein the agent is S-0885 (a collagenase inhibitor).

4323. The method of item 4215 wherein the agent is aplidine (an elongation factor-1alpha inhibitor).

4324. The method of item 4215 wherein the agent is ixabepilone (an epithilone).

4325. The method of item 4215 wherein the agent is IDN-5390 (an angiogenesis inhibitor)

4326. The method of item 4215 wherein the agent is SB-2723005 (an angiogenesis inhibitor).

4327. The method of item 4215 wherein the agent is ABT-518 (an angiogenesis inhibitor).

4328. The method of item 4215 wherein the agent is combretastatin (an angiogenesis inhibitor).

4329. The method of item 4215 wherein the agent is anecortave acetate (an angiogenesis inhibitor).

4330. The method of item 4215 wherein the agent is SB-715992 (a kinesin antagonist).

4331. The method of item 4215 wherein the agent is temsirolimus (an mTOR inhibitor).

4332. The method of item 4215 wherein the agent is adalimumab (a TNFα antagonist).

4333. The method of item 4215, wherein the composition comprises a polymer.

4334. The method of item 4215, wherein the composition comprises a polymer, and the polymer is, or comprises, a copolymer.

4335. The method of item 4215, wherein the composition comprises a polymer, and the polymer is, or comprises, a block copolymer.

4336. The method of item 4215, wherein the composition comprises a polymer, and the polymer is, or comprises, a random copolymer.

4337. The method of item 4215, wherein the composition comprises a polymer, and the polymer is, or comprises, a biodegradable polymer.

4338. The method of item 4215, wherein the composition comprises a polymer, and the polymer is, or comprises, a non-biodegradable polymer.

4339. The method of item 4215, wherein the composition comprises a polymer, and the polymer is, or comprises, a hydrophilic polymer.

4340. The method of item 4215, wherein the composition comprises a polymer, and the polymer is, or comprises, a hydrophobic polymer.

4341. The method of item 4215, wherein the composition comprises a polymer, and the polymer is, or comprises, a polymer having hydrophilic domains.

4342. The method of item 4215, wherein the composition comprises a polymer, and the polymer is, or comprises, a polymer having hydrophobic domains.

4343. The method of item 4215, wherein the composition comprises a polymer, and the polymer is, or comprises, a non-conductive polymer.

4344. The method of item 4215, wherein the composition comprises a polymer, and the polymer is, or comprises, an elastomer.

4345. The method of item 4215, wherein the composition comprises a polymer, and the polymer is, or comprises, a hydrogel.

4346. The method of item 4215, wherein the composition comprises a polymer, and the polymer is, or comprises, a silicone polymer.

4347. The method of item 4215, wherein the composition comprises a polymer, and the polymer is, or comprises, a hydrocarbon polymer.

4348. The method of item 4215, wherein the composition comprises a polymer, and the polymer is, or comprises, a styrene-derived polymer.

4349. The method of item 4215, wherein the composition comprises a polymer, and the polymer is, or comprises, a butadiene-derived polymer.

4350. The method of item 4215, wherein the composition comprises a polymer, and the polymer is, or comprises, a macromer.

4351. The method of item 4215, wherein the composition comprises a polymer, and the polymer is, or comprises, a poly(ethylene glycol) polymer.

4352. The method of item 4215, wherein the composition comprises a polymer, and the polymer is, or comprises, an amorphous polymer.

4353. The method of item 4215, wherein the composition further comprises a second pharmaceutically active agent.

4354. The method of item 4215, wherein the composition further comprises an anti-inflammatory agent.

4355. The method of item 4215, wherein the composition further comprises an agent that inhibits infection.

4356. The method of item 4215, wherein the composition further comprises an anthracycline.

4357. The method of item 4215, wherein the composition further comprises doxorubicin.

4358. The method of item 4215 wherein the composition further comprises mitoxantrone.

4359. The method of item 4215 wherein the composition further comprises a fluoropyrimidine.

4360. The method of item 4215, wherein the composition further comprises 5-fluorouracil (5-FU).

4361. The method of item 4215, wherein the composition further comprises a folic acid antagonist.

4362. The method of item 4215, wherein the composition further comprises methotrexate.

4363. The method of item 4215, wherein the composition further comprises a podophylotoxin.

4364. The method of item 4215, wherein the composition further comprises etoposide.

4365. The method of item 4215, wherein the composition further comprises camptothecin.

4366. The method of item 4215, wherein the composition further comprises a hydroxyurea.

4367. The method of item 4215, wherein the composition further comprises a platinum complex.

4368. The method of item 4215, wherein the composition further comprises cisplatin.

4369. The method of item 4215 wherein the composition further comprises an anti-thrombotic agent.

4370. The method of item 4215, wherein the composition further comprises a visualization agent.

4371. The method of item 4215, wherein the composition further comprises a visualization agent, and the visualization agent is a radiopaque material, wherein the radiopaque material comprises a metal, a halogenated compound, or a barium containing compound.

4372. The method of item 4215, wherein the composition further comprises a visualization agent, and the visualization agent is, or comprises, barium, tantalum, or technetium.

4373. The method of item 4215, wherein the composition further comprises a visualization agent, and the visualization agent is, or comprises, an MRI responsive material.

4374. The method of item 4215, wherein the composition further comprises a visualization agent, and the visualization agent is, or comprises, a gadolinium chelate.

4375. The method of item 4215, wherein the composition further comprises a visualization agent, and the visualization agent is, or comprises, iron, magnesium, manganese, copper, or chromium.

4376. The method of item 4215, wherein the composition further comprises a visualization agent, and the visualization agent is, or comprises, iron oxide compound.

4377. The method of item 4215, wherein the composition further comprises a visualization agent, and the visualization agent is, or comprises, a dye, pigment, or colorant.

4378. The method of item 4215 wherein the agent is released in effective concentrations from the composition comprising the agent by diffusion over a period ranging from the time of administration to about 90 days.

4379. The method of item 4215 wherein the agent is released in effective concentrations from the composition comprising the agent by erosion of the composition over a period ranging from the time of administration to about 90 days.

4380. The method of item 4215 wherein the composition further comprises an inflammatory cytokine.

4381. The method of item 4215 wherein the composition further comprises an agent that stimulates cell proliferation.

4382. The method of item 4215 wherein the composition further comprises a polymeric carrier.

4383. The method of item 4215 wherein the composition is in the form of a gel, paste, or spray.

4384. The method of item 4215 wherein the electrical device is partially constructed with the agent or the composition.

4385. The method of item 4215 wherein the electrical device is impregnated with the agent or the composition.

4386. The method of item 4215, wherein the agent or the composition forms a coating, and the coating directly contacts the electrical device.

4387. The method of item 4215, wherein the agent or the composition forms a coating, and the coating indirectly contacts the electrical device.

4388. The method of item 4215 wherein the agent or the composition forms a coating, and the coating partially covers the electrical device.

4389. The method of item 4215, wherein the agent or the composition forms a coating, and the coating completely covers the electrical device.

4390. The method of item 4215 wherein the agent or the composition is located within pores or holes of the electrical device.

4391. The method of item 4215 wherein the agent or the composition is located within a channel, lumen, or divet of the electrical device.

4392. The method of item 4215 wherein the electrical device further comprises an echogenic material.

4393. The method of item 4215 wherein the electrical device further comprises an echogenic material, wherein the echogenic material is in the form of a coating.

4394. The method of item 4215 wherein the electrical device is sterile.

4395. The method of item 4215 wherein the agent is delivered from the electrical device, wherein the agent is released into tissue in the vicinity of the electrical device after deployment of the electrical device.

4396. The method of item 4215 wherein the agent is delivered from the electrical device, wherein the agent is released into tissue in the vicinity of the electrical device after deployment of the electrical device, wherein the tissue is connective tissue.

4397. The method of item 4215 wherein the agent is delivered from the electrical device, wherein the agent is released into tissue in the vicinity of the electrical device after deployment of the electrical device, wherein the tissue is muscle tissue.

4398. The method of item 4215 wherein the agent is delivered from the electrical device, wherein the agent is released into tissue in the vicinity of the electrical device after deployment of the electrical device, wherein the tissue is nerve tissue.

4399. The method of item 4215 wherein the agent is delivered from the electrical device, wherein the agent is released into tissue in the vicinity of the electrical device after deployment of the electrical device, wherein the tissue is epithelium tissue.

4400. The method of item 4215 wherein the agent is delivered from the electrical device, wherein the agent is released in effective concentrations from the electrical device over a period ranging from the time of deployment of the electrical device to about 1 year.

4401. The method of item 4215 wherein the agent is delivered from the electrical device, wherein the agent is released in effective concentrations from the electrical device over a period ranging from about 1 month to 6 months.

4402. The method of item 4215 wherein the agent is delivered from the electrical device, wherein the agent is released in effective concentrations from the electrical device over a period ranging from about 1-90 days.

4403. The method of item 4215 wherein the agent is delivered from the electrical device, wherein the agent is released in effective concentrations from the electrical device at a constant rate.

4404. The method of item 4215 wherein the agent is delivered from the electrical device, wherein the agent is released in effective concentrations from the electrical device at an increasing rate.

4405. The method of item 4215 wherein the agent is delivered from the electrical device, wherein the agent is released in effective concentrations from the electrical device at a decreasing rate.

4406. The method of item 4215 wherein the agent is delivered from the electrical device, wherein the electrical device comprises about 0.01 μg to about 10 μg of the agent.

4407. The method of item 4215 wherein the agent is delivered from the electrical device, wherein the electrical device comprises about 10 μg to about 10 mg of the agent.

4408. The method of item 4215 wherein the agent is delivered from the electrical device, wherein the electrical device comprises about 10 mg to about 250 mg of the agent.

4409. The method of item 4215 wherein the agent is delivered from the electrical device, wherein the electrical device comprises about 250 mg to about 1000 mg of the agent.

4410. The method of item 4215 wherein the agent is delivered from the electrical device, wherein the electrical device comprises about 1000 mg to about 2500 mg of the agent.

4411. The method of item 4215 wherein the agent is delivered from the electrical device, wherein a surface of the electrical device comprises less than 0.01 μg of the agent per mm² of electrical device surface to which the agent is applied.

4412. The method of item 4215 wherein the agent is delivered from the electrical device, wherein a surface of the electrical device comprises about 0.01 μg to about 1 μg of the agent per mm² of electrical device surface to which the agent is applied.

4413. The method of item 4215 wherein the agent is delivered from the electrical device, wherein a surface of the electrical device comprises about 1 μg to about 10 μg of the agent per mm² of electrical device surface to which the agent is applied.

4414. The method of item 4215 wherein the agent is delivered from the electrical device, wherein a surface of the electrical device comprises about 10 μg to about 250 μg of the agent per mm² of electrical device surface to which the agent is applied.

4415. The method of item 4215 wherein the agent is delivered from the electrical device, wherein a surface of the electrical device comprises about 250 μg to about 1000 μg of the agent per mm² of electrical device surface to which the agent is applied.

4416. The method of item 4215 wherein the agent is delivered from the electrical device, wherein a surface of the electrical device comprises about 1000 μg to about 2500 μg of the agent per mm² of electrical device surface to which the agent is applied.

4417. The method of item 4215, wherein the electrical device further comprises a coating, and the coating is a uniform coating.

4418. The method of item 4215, wherein the electrical device further comprises a coating, and the coating is a non-uniform coating.

4419. The method of item 4215, wherein the electrical device further comprises a coating, and the coating is a discontinuous coating.

4420. The method of item 4215, wherein the electrical device further comprises a coating, and the coating is a patterned coating.

4421. The method of item 4215, wherein the electrical device further comprises a coating, and the coating has a thickness of 100 μm or less.

4422. The method of item 4215, wherein the electrical device further comprises a coating, and the coating has a thickness of 10 μm or less.

4423. The method of item 4215, wherein the electrical device further comprises a coating, and the coating adheres to the surface of the electrical device upon deployment of the electrical device.

4424. The method of item 4215, wherein the electrical device further comprises a coating, and the coating is stable at room temperature for a period of at least 1 year.

4425. The method of item 4215, wherein the electrical device further comprises a coating, and the agent is present in the coating in an amount ranging between about 0.0001% to about 1% by weight.

4426. The method of item 4215, wherein the electrical device further comprises a coating, and the agent is present in the coating in an amount ranging between about 1% to about 10% by weight.

4427. The method of item 4215, wherein the electrical device further comprises a coating, and the agent is present in the coating in an amount ranging between about 10% to about 25% by weight.

4428. The method of item 4215, wherein the electrical device further comprises a coating, and the agent is present in the coating in an amount ranging between about 25% to about 70% by weight.

4429. The method of item 4215, wherein the electrical device further comprises a coating, and the coating comprises a polymer.

4430. The method of item 4215, wherein the electrical device comprises a first coating having a first composition and a second coating having a second composition.

4431. The method of item 4215, wherein the electrical device comprises a first coating having a first composition and a second coating having a second composition, wherein the first composition and the second composition are different.

4432. The method of item 4215 wherein the agent or the composition is affixed to the electrical device.

4433. The method of item 4215 wherein the agent or the composition is covalently attached to the electrical device.

4434. The method of item 4215 wherein the agent or the composition is non-covalently attached to the electrical device.

4435. The method of item 4215 wherein the electrical device comprises a coating that absorbs the agent or the composition.

4436. The method of item 4215 wherein the electrical device is interweaved with a thread composed of, or coated with, the agent or the composition.

4437. The method of item 4215 wherein a portion of the electrical device is covered with a sleeve that contains the agent or the composition.

4438. The method of item 4215 wherein the electrical device is completely covered with a sleeve that contains the agent or the composition.

4439. The method of item 4215 wherein a portion of the electrical device is covered with a mesh that contains the agent or the composition.

4440. The method of item 4215 wherein the electrical device is completely covered with a mesh that contains the agent or the composition.

4441. The method of item 4215 wherein the agent or the composition is applied to the electrical device surface prior to the placing of the electrical device into the host.

4442. The method of item 4215 wherein the agent or the composition is applied to the electrical device surface during the placing of the electrical device into the host.

4443. The method of item 4215 wherein the agent or the composition is applied to the electrical device surface immediately after the placing of the electrical device into the host.

4444. The method of item 4215 wherein the agent or the composition is applied to the surface of the tissue in the host surrounding the electrical device prior to the placing of the electrical device into the host.

4445. The method of item 4215 wherein the agent or the composition is applied to the surface of the tissue in the host surrounding the electrical device during the placing of the electrical device into the host.

4446. The method of item 4215 wherein the agent or the composition is applied to the surface of the tissue in the host surrounding the electrical device immediately after the placing of the electrical device into the host.

4447. The method of item 4215 wherein the agent or the composition is topically applied into the anatomical space where the electrical device is placed.

4448. The method of item 4215 wherein the agent or the composition is percutaneously injected into the tissue in the host surrounding the electrical device.

4449. The method for inhibiting scarring of any one of items 4215-4448 wherein the neurostimulator comprises an intracranially implantable electrical control module and an electrode.

4450. The method for inhibiting scarring of any one of items 4215-4448 wherein the neurostimulator comprises a sensor and an electrode.

4451. A method for inhibiting scarring comprising placing a vagal nerve stimulator for treating epilepsy (i.e., an electrical device) and an anti-scarring agent or a composition comprising an ant-scarring agent into an animal host, wherein the agent inhibits scarring.

4452. The method of item 4451 wherein the agent is an adensosine A2A receptor antagonist.

4453. The method of item 4451 wherein the agent is an AKT inhibitor.

4454. The method of item 4451 wherein the agent is an alpha 2 integrin antagonist, wherein the alpha 2 integrin antagonist is Pharmaprojects No. 5754 (Merck KgaA).

4455. The method of item 4451 wherein the agent is an alpha 4 integrin antagonist.

4456. The method of item 4451 wherein the agent is an alpha 7 nicotinic receptor agonist.

4457. The method of item 4451 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

4458. The method of item 4451 wherein the agent is an apoptosis antagonist.

4459. The method of item 4451 wherein the agent is an apoptosis activator.

4460. The method of item 4451 wherein the agent is a beta 1 integrin antagonist.

4461. The method of item 4451 wherein the agent is a beta tubulin inhibitor.

4462. The method of item 4451 wherein the agent is a blocker of enzyme production in Hepatitis C.

4463. The method of item 4451 wherein the agent is a Bruton's tyrosine kinase inhibitor.

4464. The method of item 4451 wherein the agent is a calcineurin inhibitor.

4465. The method of item 4451 wherein the agent is a caspase 3 inhibitor.

4466. The method of item 4451 wherein the agent is a CC chemokine receptor antagonist.

4467. The method of item 4451 wherein the agent is a cell cycle inhibitor selected from the group consisting of SNS-595 (Sunesis), synthadotin, KRX-0403, and an analogue or derivative thereof.

4468. The method of item 4451 wherein the agent is a cathepsin B inhibitor.

4469. The method of item 4451 wherein the agent is a cathepsin K inhibitor, wherein the cathepsin K inhibitor is 462795 (GlaxoSmithKline), INPL-022-D6 (Amura Therapeutics), or an analogue or derivative thereof.

4470. The method of item 4451 wherein the agent is a cathepsin L inhibitor.

4471. The method of item 4451 wherein the agent is a CD40 antagonist.

4472. The method of item 4451 wherein the agent is a chemokine receptor agonist.

4473. The method of item 4451 wherein the agent is a chymase inhibitor.

4474. The method of item 4451 wherein the agent is a collagenase antagonist.

4475. The method of item 4451 wherein the agent is a CXCR antagonist.

4476. The method of item 4451 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

4477. The method of item 4451 wherein the agent is a cyclooxygenase I inhibitor.

4478. The method of item 4451 wherein the agent is a DHFR inhibitor.

4479. The method of item 4451 wherein the agent is a dual integrin inhibitor.

4480. The method of item 4451 wherein the agent is an elastase inhibitor.

4481. The method of item 4451 wherein the agent is an elongation factor-1 alpha inhibitor.

4482. The method of item 4451 wherein the agent is an endothelial growth factor antagonist.

4483. The method of item 4451 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

4484. The method of item 4451 wherein the agent is an endotoxin antagonist.

4485. The method of item 4451 wherein the agent is an epothilone and tubulin binder

4486. The method of item 4451 wherein the agent is an estrogen receptor antagonist.

4487. The method of item 4451 wherein the agent is an FGF inhibitor.

4488. The method of item 4451 wherein the agent is a farnexyl transferase inhibitor.

4489. The method of item 4451 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

4490. The method of item 4451 wherein the agent is an FLT-3 kinase inhibitor.

4491. The method of item 4451 wherein the agent is an FGF receptor kinase inhibitor.

4492. The method of item 4451 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

4493. The method of item 4451 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

4494. The method of item 4451 wherein the agent is a histone deacetylase inhibitor.

4495. The method of item 4451 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

4496. The method of item 4451 wherein the agent is an ICAM inhibitor.

4497. The method of item 4451 wherein the agent is an IL, ICE and IRAK antagonist, wherein the antagonist is a CJ-14877, CP-424174 (Pfizer), NF-61 (Negma-Lerads), and an analogue or derivative thereof.

4498. The method of item 4451 wherein the agent is an IL-2 inhibitor.

4499. The method of item 4451 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

4500. The method of item 4451 wherein the agent is an IMPDH (inosine monophosphate).

4501. The method of item 4451 wherein the agent is an integrin antagonist.

4502. The method of item 4451 wherein the agent is an interleukin antagonist.

4503. The method of item 4451 wherein the agent is an inhibitor of type III receptor tyrosine kinase.

4504. The method of item 4451 wherein the agent is an irreversible inhibitor of enzyme methionine aminopeptidase type 2.

4505. The method of item 4451 wherein the agent is an isozyme selective delta protein kinase C inhibitor.

4506. The method of item 4451 wherein the agent a JAK3 enzyme inhibitor.

4507. The method of item 4451 wherein the agent is a JNK inhibitor.

4508. The method of item 4451 wherein the agent is a kinase inhibitor.

4509. The method of item 4451 wherein the agent is kinesin antagonist.

4510. The method of item 4451 wherein the agent is a kinesin antagonist.

4511. The method of item 4451 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

4512. The method of item 4451 wherein the agent is an MAP kinase inhibitor.

4513. The method of item 4451 wherein the agent is a matrix metalloproteinase inhibitor.

4514. The method of item 4451 wherein the agent is an MCP-CCR2 inhibitor.

4515. The method of item 4451 wherein the agent is an mTOR inhibitor.

4516. The method of item 4451 wherein the agent is an mTOR kinase inhibitor.

4517. The method of item 4451 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

4518. The method of item 4451 wherein the agent is an MIF inhibitor.

4519. The method of item 4451 wherein the agent is an MMP inhibitor.

4520. The method of item 4451 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

4521. The method of item 4451 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

4522. The method of item 4451 wherein the agent is a nitric oxide agonist.

4523. The method of item 4451 wherein the agent is an ornithine decarboxylase inhibitor.

4524. The method of item 4451 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

4525. The method of item 4451 wherein the agent is a palmitoyl-protein thioesterase inhibitor.

4526. The method of item 4451 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

4527. The method of item 4451 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

4528. The method of item 4451 wherein the agent is a phosphatase inhibitor.

4529. The method of item 4451 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

4530. The method of item 4451 wherein the agent is a PKC inhibitor.

4531. The method of item 4451 wherein the agent is a platelet activating factor antagonist.

4532. The method of item 4451 wherein the agent is a platelet-derived growth factor receptor kinase inhibitor.

4533. The method of item 4451 wherein the agent is a prolyl hydroxylase inhibitor.

4534. The method of item 4451 wherein the agent is a polymorphonuclear neutrophil inhibitor.

4535. The method of item 4451 wherein the agent is a protein kinase B inhibitor.

4536. The method of item 4451 wherein the agent is a protein kinase C stimulant.

4537. The method of item 4451 wherein the agent is a purine nucleoside analogue.

4538. The method of item 4451 wherein the agent is a purinoreceptor P2X antagonist.

4539. The method of item 4451 wherein the agent is a Raf kinase inhibitor.

4540. The method of item 4451 wherein the agent is a reversible inhibitor of ErbB1 and ErbB2.

4541. The method of item 4451 wherein the agent is a ribonucleoside triphosphate reductase inhibitor.

4542. The method of item 4451 wherein the agent is an SDF-1 antagonist.

4543. The method of item 4451 wherein the agent is a sheddase inhibitor.

4544. The method of item 4451 wherein the agent is an SRC inhibitor.

4545. The method of item 4451 wherein the agent is a stromelysin inhibitor.

4546. The method of item 4451 wherein the agent is an Syk kinase inhibitor.

4547. The method of item 4451 wherein the agent is a telomerase inhibitor.

4548. The method of item 4451 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

4549. The method of item 4451 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

4550. The method of item 4451 wherein the agent is a Toll receptor inhibitor.

4551. The method of item 4451 wherein the agent is a tubulin antagonist.

4552. The method of item 4451 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

4553. The method of item 4451 wherein the agent is a VEGF inhibitor.

4554. The method of item 4451 wherein the agent is a vitamin D receptor agonist.

4555. The method of item 4451 wherein the agent is ZD-6474 (an angiogenesis inhibitor).

4556. The method of item 4451 wherein the agent is AP-23573 (an mTOR inhibitor).

4557. The method of item 4451 wherein the agent is synthadotin (a tubulin antagonist).

4558. The method of item 4451 wherein the agent is S-0885 (a collagenase inhibitor).

4559. The method of item 4451 wherein the agent is aplidine (an elongation factor-1 alpha inhibitor).

4560. The method of item 4451 wherein the agent is ixabepilone (an epithilone).

4561. The method of item 4451 wherein the agent is IDN-5390 (an angiogenesis inhibitor).

4562. The method of item 4451 wherein the agent is SB-2723005 (an angiogenesis inhibitor).

4563. The method of item 4451 wherein the agent is ABT-518 (an angiogenesis inhibitor).

4564. The method of item 4451 wherein the agent is combretastatin (an angiogenesis inhibitor).

4565. The method of item 4451 wherein the agent is anecortave acetate (an angiogenesis inhibitor).

4566. The method of item 4451 wherein the agent is SB-715992 (a kinesin antagonist).

4567. The method of item 4451 wherein the agent is temsirolimus (an mTOR inhibitor).

4568. The method of item 4451 wherein the agent is adalimumab (a TNFα antagonist).

4569. The method of item 4451, wherein the composition comprises a polymer.

4570. The method of item 4451, wherein the composition comprises a polymer, and the polymer is, or comprises, a copolymer.

4571. The method of item 4451, wherein the composition comprises a polymer, and the polymer is, or comprises, a block copolymer.

4572. The method of item 4451, wherein the composition comprises a polymer, and the polymer is, or comprises, a random copolymer.

4573. The method of item 4451, wherein the composition comprises a polymer, and the polymer is, or comprises, a biodegradable polymer.

4574. The method of item 4451, wherein the composition comprises a polymer, and the polymer is, or comprises, a non-biodegradable polymer.

4575. The method of item 4451, wherein the composition comprises a polymer, and the polymer is, or comprises, a hydrophilic polymer.

4576. The method of item 4451, wherein the composition comprises a polymer, and the polymer is, or comprises, a hydrophobic polymer.

4577. The method of item 4451, wherein the composition comprises a polymer, and the polymer is, or comprises, a polymer having hydrophilic domains.

4578. The method of item 4451, wherein the composition comprises a polymer, and the polymer is, or comprises, a polymer having hydrophobic domains.

4579. The method of item 4451, wherein the composition comprises a polymer, and the polymer is, or comprises, a non-conductive polymer.

4580. The method of item 4451, wherein the composition comprises a polymer, and the polymer is, or comprises, an elastomer.

4581. The method of item 4451, wherein the composition comprises a polymer, and the polymer is, or comprises, a hydrogel.

4582. The method of item 4451, wherein the composition comprises a polymer, and the polymer is, or comprises, a silicone polymer.

4583. The method of item 4451, wherein the composition comprises a polymer, and the polymer is, or comprises, a hydrocarbon polymer.

4584. The method of item 4451, wherein the composition comprises a polymer, and the polymer is, or comprises, a styrene-derived polymer.

4585. The method of item 4451, wherein the composition comprises a polymer, and the polymer is, or comprises, a butadiene-derived polymer.

4586. The method of item 4451, wherein the composition comprises a polymer, and the polymer is, or comprises, a macromer.

4587. The method of item 4451, wherein the composition comprises a polymer, and the polymer is, or comprises, a poly(ethylene glycol) polymer.

4588. The method of item 4451, wherein the composition comprises a polymer, and the polymer is, or comprises, an amorphous polymer.

4589. The method of item 4451, wherein the composition further comprises a second pharmaceutically active agent.

4590. The method of item 4451, wherein the composition further comprises an anti-inflammatory agent.

4591. The method of item 4451, wherein the composition further comprises an agent that inhibits infection.

4592. The method of item 4451, wherein the composition further comprises an anthracycline.

4593. The method of item 4451, wherein the composition further comprises doxorubicin.

4594. The method of item 4451 wherein the composition further comprises mitoxantrone.

4595. The method of item 4451 wherein the composition further comprises a fluoropyrimidine.

4596. The method of item 4451, wherein the composition further comprises 5-fluorouracil (5-FU).

4597. The method of item 4451, wherein the composition further comprises a folic acid antagonist.

4598. The method of item 4451, wherein the composition further comprises methotrexate.

4599. The method of item 4451, wherein the composition further comprises a podophylotoxin.

4600. The method of item 4451, wherein the composition further comprises etoposide.

4601. The method of item 4451, wherein the composition further comprises camptothecin.

4602. The method of item 4451, wherein the composition further comprises a hydroxyurea.

4603. The method of item 4451, wherein the composition further comprises a platinum complex.

4604. The method of item 4451, wherein the composition further comprises cisplatin.

4605. The method of item 4451 wherein the composition further comprises an anti-thrombotic agent.

4606. The method of item 4451, wherein the composition further comprises a visualization agent.

4607. The method of item 4451, wherein the composition further comprises a visualization agent, and the visualization agent is a radiopaque material, wherein the radiopaque material comprises a metal, a halogenated compound, or a barium containing compound.

4608. The method of item 4451, wherein the composition further comprises a visualization agent, and the visualization agent is, or comprises, barium, tantalum, or technetium.

4609. The method of item 4451, wherein the composition further comprises a visualization agent, and the visualization agent is, or comprises, an MRI responsive material.

4610. The method of item 4451, wherein the composition further comprises a visualization agent, and the visualization agent is, or comprises, a gadolinium chelate.

4611. The method of item 4451, wherein the composition further comprises a visualization agent, and the visualization agent is, or comprises, iron, magnesium, manganese, copper, or chromium.

4612. The method of item 4451, wherein the composition further comprises a visualization agent, and the visualization agent is, or comprises, iron oxide compound.

4613. The method of item 4451, wherein the composition further comprises a visualization agent, and the visualization agent is, or comprises, a dye, pigment, or colorant.

4614. The method of item 4451 wherein the agent is released in effective concentrations from the composition comprising the agent by diffusion over a period ranging from the time of administration to about 90 days.

4615. The method of item 4451 wherein the agent is released in effective concentrations from the composition comprising the agent by erosion of the composition over a period ranging from the time of administration to about 90 days.

4616. The method of item 4451 wherein the composition further comprises an inflammatory cytokine.

4617. The method of item 4451 wherein the composition further comprises an agent that stimulates cell proliferation.

4618. The method of item 4451 wherein the composition further comprises a polymeric carrier.

4619. The method of item 4451 wherein the composition is in the form of a gel, paste, or spray.

4620. The method of item 4451 wherein the electrical device is partially constructed with the agent or the composition.

4621. The method of item 4451 wherein the electrical device is impregnated with the agent or the composition.

4622. The method of item 4451, wherein the agent or the composition forms a coating, and the coating directly contacts the electrical device.

4623. The method of item 4451, wherein the agent or the composition forms a coating, and the coating indirectly contacts the electrical device.

4624. The method of item 4451 wherein the agent or the composition forms a coating, and the coating partially covers the electrical device.

4625. The method of item 4451, wherein the agent or the composition forms a coating, and the coating completely covers the electrical device.

4626. The method of item 4451 wherein the agent or the composition is located within pores or holes of the electrical device.

4627. The method of item 4451 wherein the agent or the composition is located within a channel, lumen, or divet of the electrical device.

4628. The method of item 4451 wherein the electrical device further comprises an echogenic material.

4629. The method of item 4451 wherein the electrical device further comprises an echogenic material, wherein the echogenic material is in the form of a coating.

4630. The method of item 4451 wherein the electrical device is sterile.

4631. The method of item 4451 wherein the agent is delivered from the electrical device, wherein the agent is released into tissue in the vicinity of the electrical device after deployment of the electrical device.

4632. The method of item 4451 wherein the agent is delivered from the electrical device, wherein the agent is released into tissue in the vicinity of the electrical device after deployment of the electrical device, wherein the tissue is connective tissue.

4633. The method of item 4451 wherein the agent is delivered from the electrical device, wherein the agent is released into tissue in the vicinity of the electrical device after deployment of the electrical device, wherein the tissue is muscle tissue.

4634. The method of item 4451 wherein the agent is delivered from the electrical device, wherein the agent is released into tissue in the vicinity of the electrical device after deployment of the electrical device, wherein the tissue is nerve tissue.

4635. The method of item 4451 wherein the agent is delivered from the electrical device, wherein the agent is released into tissue in the vicinity of the electrical device after deployment of the electrical device, wherein the tissue is epithelium tissue.

4636. The method of item 4451 wherein the agent is delivered from the electrical device, wherein the agent is released in effective concentrations from the electrical device over a period ranging from the time of deployment of the electrical device to about 1 year.

4637. The method of item 4451 wherein the agent is delivered from the electrical device, wherein the agent is released in effective concentrations from the electrical device over a period ranging from about 1 month to 6 months.

4638. The method of item 4451 wherein the agent is delivered from the electrical device, wherein the agent is released in effective concentrations from the electrical device over a period ranging from about 1-90 days.

4639. The method of item 4451 wherein the agent is delivered from the electrical device, wherein the agent is released in effective concentrations from the electrical device at a constant rate.

4640. The method of item 4451 wherein the agent is delivered from the electrical device, wherein the agent is released in effective concentrations from the electrical device at an increasing rate.

4641. The method of item 4451 wherein the agent is delivered from the electrical device, wherein the agent is released in effective concentrations from the electrical device at a decreasing rate.

4642. The method of item 4451 wherein the agent is delivered from the electrical device, wherein the electrical device comprises about 0.01 μg to about 10 μg of the agent.

4643. The method of item 4451 wherein the agent is delivered from the electrical device, wherein the electrical device comprises about 10 μg to about 10 mg of the agent.

4644. The method of item 4451 wherein the agent is delivered from the electrical device, wherein the electrical device comprises about 10 mg to about 250 mg of the agent.

4645. The method of item 4451 wherein the agent is delivered from the electrical device, wherein the electrical device comprises about 250 mg to about 1000 mg of the agent.

4646. The method of item 4451 wherein the agent is delivered from the electrical device, wherein the electrical device comprises about 1000 mg to about 2500 mg of the agent.

4647. The method of item 4451 wherein the agent is delivered from the electrical device, wherein a surface of the electrical device comprises less than 0.01 μg of the agent per mm² of electrical device surface to which the agent is applied.

4648. The method of item 4451 wherein the agent is delivered from the electrical device, wherein a surface of the electrical device comprises about 0.01 μg to about 1 μg of the agent per mm² of electrical device surface to which the agent is applied.

4649. The method of item 4451 wherein the agent is delivered from the electrical device, wherein a surface of the electrical device comprises about 1 μg to about 10 μg of the agent per mm² of electrical device surface to which the agent is applied.

4650. The method of item 4451 wherein the agent is delivered from the electrical device, wherein a surface of the electrical device comprises about 10 μg to about 250 μg of the agent per mm² of electrical device surface to which the agent is applied.

4651. The method of item 4451 wherein the agent is delivered from the electrical device, wherein a surface of the electrical device comprises about 250 μg to about 1000 μg of the agent per mm² of electrical device surface to which the agent is applied.

4652. The method of item 4451 wherein the agent is delivered from the electrical device, wherein a surface of the electrical device comprises about 1000 μg to about 2500 μg of the agent per mm² of electrical device surface to which the agent is applied.

4653. The method of item 4451, wherein the electrical device further comprises a coating, and the coating is a uniform coating.

4654. The method of item 4451, wherein the electrical device further comprises a coating, and the coating is a non-uniform coating.

4655. The method of item 4451, wherein the electrical device further comprises a coating, and the coating is a discontinuous coating.

4656. The method of item 4451, wherein the electrical device further comprises a coating, and the coating is a patterned coating.

4657. The method of item 4451, wherein the electrical device further comprises a coating, and the coating has a thickness of 100 μm or less.

4658. The method of item 4451, wherein the electrical device further comprises a coating, and the coating has a thickness of 10 μm or less.

4659. The method of item 4451, wherein the electrical device further comprises a coating, and the coating adheres to the surface of the electrical device upon deployment of the electrical device.

4660. The method of item 4451, wherein the electrical device further comprises a coating, and the coating is stable at room temperature for a period of at least 1 year.

4661. The method of item 4451, wherein the electrical device further comprises a coating, and the agent is present in the coating in an amount ranging between about 0.0001% to about 1% by weight.

4662. The method of item 4451, wherein the electrical device further comprises a coating, and the agent is present in the coating in an amount ranging between about 1% to about 10% by weight.

4663. The method of item 4451, wherein the electrical device further comprises a coating, and the agent is present in the coating in an amount ranging between about 10% to about 25% by weight.

4664. The method of item 4451, wherein the electrical device further comprises a coating, and the agent is present in the coating in an amount ranging between about 25% to about 70% by weight.

4665. The method of item 4451, wherein the electrical device further comprises a coating, and the coating comprises a polymer.

4666. The method of item 4451, wherein the electrical device comprises a first coating having a first composition and a second coating having a second composition.

4667. The method of item 4451, wherein the electrical device comprises a first coating having a first composition and a second coating having a second composition, wherein the first composition and the second composition are different.

4668. The method of item 4451 wherein the agent or the composition is affixed to the electrical device.

4669. The method of item 4451 wherein the agent or the composition is covalently attached to the electrical device.

4670. The method of item 4451 wherein the agent or the composition is non-covalently attached to the electrical device.

4671. The method of item 4451 wherein the electrical device comprises a coating that absorbs the agent or the composition.

4672. The method of item 4451 wherein the electrical device is interweaved with a thread composed of, or coated with, the agent or the composition.

4673. The method of item 4451 wherein a portion of the electrical device is covered with a sleeve that contains the agent or the composition.

4674. The method of item 4451 wherein the electrical device is completely covered with a sleeve that contains the agent or the composition.

4675. The method of item 4451 wherein a portion of the electrical device is covered with a mesh that contains the agent or the composition.

4676. The method of item 4451 wherein the electrical device is completely covered with a mesh that contains the agent or the composition.

4677. The method of item 4451 wherein the agent or the composition is applied to the electrical device surface prior to the placing of the electrical device into the host.

4678. The method of item 4451 wherein the agent or the composition is applied to the electrical device surface during the placing of the electrical device into the host.

4679. The method of item 4451 wherein the agent or the composition is applied to the electrical device surface immediately after the placing of the electrical device into the host.

4680. The method of item 4451 wherein the agent or the composition is applied to the surface of the tissue in the host surrounding the electrical device prior to the placing of the electrical device into the host.

4681. The method of item 4451 wherein the agent or the composition is applied to the surface of the tissue in the host surrounding the electrical device during the placing of the electrical device into the host.

4682. The method of item 4451 wherein the agent or the composition is applied to the surface of the tissue in the host surrounding the electrical device immediately after the placing of the electrical device into the host.

4683. The method of item 4451 wherein the agent or the composition is topically applied into the anatomical space where the electrical device is placed.

4684. The method of item 4451 wherein the agent or the composition is percutaneously injected into the tissue in the host surrounding the electrical device.

4685. A method for inhibiting scarring comprising placing a vagal nerve stimulator (i.e., an electrical device) and an anti-scarring agent or a composition comprising an ant-scarring agent into an animal host, wherein the agent inhibits scarring.

4686. The method of item 4685 wherein the agent is an adensosine A2A receptor antagonist.

4687. The method of item 4685 wherein the agent is an AKT inhibitor.

4688. The method of item 4685 wherein the agent is an alpha 2 integrin antagonist, wherein the alpha 2 integrin antagonist is Pharmaprojects No. 5754 (Merck KgaA).

4689. The method of item 4685 wherein the agent is an alpha 4 integrin antagonist.

4690. The method of item 4685 wherein the agent is an alpha 7 nicotinic receptor agonist.

4691. The method of item 4685 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

4692. The method of item 4685 wherein the agent is an apoptosis antagonist.

4693. The method of item 4685 wherein the agent is an apoptosis activator.

4694. The method of item 4685 wherein the agent is a beta 1 integrin antagonist.

4695. The method of item 4685 wherein the agent is a beta tubulin inhibitor.

4696. The method of item 4685 wherein the agent is a blocker of enzyme production in Hepatitis C.

4697. The method of item 4685 wherein the agent is a Bruton's tyrosine kinase inhibitor.

4698. The method of item 4685 wherein the agent is a calcineurin inhibitor.

4699. The method of item 4685 wherein the agent is a caspase 3 inhibitor.

4700. The method of item 4685 wherein the agent is a CC chemokine receptor antagonist.

4701. The method of item 4685 wherein the agent is a cell cycle inhibitor selected from the group consisting of SNS-595 (Sunesis), synthadotin, KRX-0403, and an analogue or derivative thereof.

4702. The method of item 4685 wherein the agent is a cathepsin B inhibitor.

4703. The method of item 4685 wherein the agent is a cathepsin K inhibitor, wherein the cathepsin K inhibitor is 462795 (GlaxoSmithKline), INPL-022-D6 (Amura Therapeutics), or an analogue or derivative thereof.

4704. The method of item 4685 wherein the agent is a cathepsin L inhibitor.

4705. The method of item 4685 wherein the agent is a CD40 antagonist.

4706. The method of item 4685 wherein the agent is a chemokine receptor agonist.

4707. The method of item 4685 wherein the agent is a chymase inhibitor.

4708. The method of item 4685 wherein the agent is a collagenase antagonist.

4709. The method of item 4685 wherein the agent is a CXCR antagonist.

4710. The method of item 4685 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

4711. The method of item 4685 wherein the agent is a cyclooxygenase 1 inhibitor.

4712. The method of item 4685 wherein the agent is a DHFR inhibitor.

4713. The method of item 4685 wherein the agent is a dual integrin inhibitor.

4714. The method of item 4685 wherein the agent is an elastase inhibitor.

4715. The method of item 4685 wherein the agent is an elongation factor-1 alpha inhibitor.

4716. The method of item 4685 wherein the agent is an endothelial growth factor antagonist.

4717. The method of item 4685 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

4718. The method of item 4685 wherein the agent is an endotoxin antagonist.

4719. The method of item 4685 wherein the agent is an epothilone and tubulin binder.

4720. The method of item 4685 wherein the agent is an estrogen receptor antagonist.

4721. The method of item 4685 wherein the agent is an FGF inhibitor.

4722. The method of item 4685 wherein the agent is a farnexyl transferase inhibitor.

4723. The method of item 4685 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

4724. The method of item 4685 wherein the agent is an FLT-3 kinase inhibitor.

4725. The method of item 4685 wherein the agent is an FGF receptor kinase inhibitor.

4726. The method of item 4685 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

4727. The method of item 4685 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

4728. The method of item 4685 wherein the agent is a histone deacetylase inhibitor.

4729. The method of item 4685 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

4730. The method of item 4685 wherein the agent is an ICAM inhibitor.

4731. The method of item 4685 wherein the agent is an IL, ICE and IRAK antagonist, wherein the antagonist is a CJ-14877, CP-424174 (Pfizer), NF-61 (Negma-Lerads), and an analogue or derivative thereof.

4732. The method of item 4685 wherein the agent is an IL-2 inhibitor.

4733. The method of item 4685 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), GO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

4734. The method of item 4685 wherein the agent is an IMPDH (inosine monophosphate).

4735. The method of item 4685 wherein the agent is an integrin antagonist.

4736. The method of item 4685 wherein the agent is an interleukin antagonist.

4737. The method of item 4685 wherein the agent is an inhibitor of type III receptor tyrosine kinase.

4738. The method of item 4685 wherein the agent is an irreversible inhibitor of enzyme methionine aminopeptidase type 2.

4739. The method of item 4685 wherein the agent is an isozyme selective delta protein kinase C inhibitor.

4740. The method of item 4685 wherein the agent a JAK3 enzyme inhibitor.

4741. The method of item 4685 wherein the agent is a JNK inhibitor.

4742. The method of item 4685 wherein the agent is a kinase inhibitor.

4743. The method of item 4685 wherein the agent is kinesin antagonist.

4744. The method of item 4685 wherein the agent is a kinesin antagonist.

4745. The method of item 4685 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

4746. The method of item 4685 wherein the agent is an MAP kinase inhibitor.

4747. The method of item 4685 wherein the agent is a matrix metalloproteinase inhibitor.

4748. The method of item 4685 wherein the agent is an MCP-CCR2 inhibitor.

4749. The method of item 4685 wherein the agent is an mTOR inhibitor.

4750. The method of item 4685 wherein the agent is an mTOR kinase inhibitor.

4751. The method of item 4685 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

4752. The method of item 4685 wherein the agent is an MIF inhibitor.

4753. The method of item 4685 wherein the agent is an MMP inhibitor.

4754. The method of item 4685 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

4755. The method of item 4685 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

4756. The method of item 4685 wherein the agent is a nitric oxide agonist.

4757. The method of item 4685 wherein the agent is an ornithine decarboxylase inhibitor.

4758. The method of item 4685 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

4759. The method of item 4685 wherein the agent is a palmitoyl-protein thioesterase inhibitor.

4760. The method of item 4685 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

4761. The method of item 4685 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-734 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

4762. The method of item 4685 wherein the agent is a phosphatase inhibitor.

4763. The method of item 4685 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

4764. The method of item 4685 wherein the agent is a PKC inhibitor.

4765. The method of item 4685 wherein the agent is a platelet activating factor antagonist.

4766. The method of item 4685 wherein the agent is a platelet-derived growth factor receptor kinase inhibitor.

4767. The method of item 4685 wherein the agent is a prolyl hydroxylase inhibitor.

4768. The method of item 4685 wherein the agent is a polymorphonuclear neutrophil inhibitor.

4769. The method of item 4685 wherein the agent is a protein kinase B inhibitor.

4770. The method of item 4685 wherein the agent is a protein kinase C stimulant.

4771. The method of item 4685 wherein the agent is a purine nucleoside analogue.

4772. The method of item 4685 wherein the agent is a purinoreceptor P2X antagonist.

4773. The method of item 4685 wherein the agent is a Raf kinase inhibitor.

4774. The method of item 4685 wherein the agent is a reversible inhibitor of ErbB1 and ErbB2.

4775. The method of item 4685 wherein the agent is a ribonucleoside triphosphate reductase inhibitor.

4776. The method of item 4685 wherein the agent is an SDF-1 antagonist.

4777. The method of item 4685 wherein the agent is a sheddase inhibitor.

4778. The method of item 4685 wherein the agent is an SRC inhibitor.

4779. The method of item 4685 wherein the agent is a stromelysin inhibitor.

4780. The method of item 4685 wherein the agent is an Syk kinase inhibitor.

4781. The method of item 4685 wherein the agent is a telomerase inhibitor.

4782. The method of item 4685 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

4783. The method of item 4685 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

4784. The method of item 4685 wherein the agent is a Toll receptor inhibitor.

4785. The method of item 4685 wherein the agent is a tubulin antagonist.

4786. The method of item 4685 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

4787. The method of item 4685 wherein the agent is a VEGF inhibitor.

4788. The method of item 4685 wherein the agent is a vitamin D receptor agonist.

4789. The method of item 4685 wherein the agent is ZD-6474 (an angiogenesis inhibitor).

4790. The method of item 4685 wherein the agent is AP-23573 (an mTOR inhibitor).

4791. The method of item 4685 wherein the agent is synthadotin (a tubulin antagonist).

4792. The method of item 4685 wherein the agent is S-0885 (a collagenase inhibitor).

4793. The method of item 4685 wherein the agent is aplidine (an elongation factor-1 alpha inhibitor).

4794. The method of item 4685 wherein the agent is ixabepilone (an epithilone).

4795. The method of item 4685 wherein the agent is IDN-5390 (an angiogenesis inhibitor)

4796. The method of item 4685 wherein the agent is SB-2723005 (an angiogenesis inhibitor).

4797. The method of item 4685 wherein the agent is ABT-518 (an angiogenesis inhibitor).

4798. The method of item 4685 wherein the agent is combretastatin (an angiogenesis inhibitor).

4799. The method of item 4685 wherein the agent is anecortave acetate (an angiogenesis inhibitor).

4800. The method of item 4685 wherein the agent is SB-715992 (a kinesin antagonist).

4801. The method of item 4685 wherein the agent is temsirolimus (an mTOR inhibitor).

4802. The method of item 4685 wherein the agent is adalimumab (a TNFα antagonist).

4803. The method of item 4685, wherein the composition comprises a polymer.

4804. The method of item 4685, wherein the composition comprises a polymer, and the polymer is, or comprises, a copolymer.

4805. The method of item 4685, wherein the composition comprises a polymer, and the polymer is, or comprises, a block copolymer.

4806. The method of item 4685, wherein the composition comprises a polymer, and the polymer is, or comprises, a random copolymer.

4807. The method of item 4685, wherein the composition comprises a polymer, and the polymer is, or comprises, a biodegradable polymer.

4808. The method of item 4685, wherein the composition comprises a polymer, and the polymer is, or comprises, a non-biodegradable polymer.

4809. The method of item 4685, wherein the composition comprises a polymer, and the polymer is, or comprises, a hydrophilic polymer.

4810. The method of item 4685, wherein the composition comprises a polymer, and the polymer is, or comprises, a hydrophobic polymer.

4811. The method of item 4685, wherein the composition comprises a polymer, and the polymer is, or comprises, a polymer having hydrophilic domains.

4812. The method of item 4685, wherein the composition comprises a polymer, and the polymer is, or comprises, a polymer having hydrophobic domains

4813. The method of item 4685, wherein the composition comprises a polymer, and the polymer is, or comprises, a non-conductive polymer.

4814. The method of item 4685, wherein the composition comprises a polymer, and the polymer is, or comprises, an elastomer.

4815. The method of item 4685, wherein the composition comprises a polymer, and the polymer is, or comprises, a hydrogel.

4816. The method of item 4685, wherein the composition comprises a polymer, and the polymer is, or comprises, a silicone polymer.

4817. The method of item 4685, wherein the composition comprises a polymer, and the polymer is, or comprises, a hydrocarbon polymer.

4818. The method of item 4685, wherein the composition comprises a polymer, and the polymer is, or comprises, a styrene-derived polymer.

4819. The method of item 4685, wherein the composition comprises a polymer, and the polymer is, or comprises, a butadiene-derived polymer.

4820. The method of item 4685, wherein the composition comprises a polymer, and the polymer is, or comprises, a macromer.

4821. The method of item 4685, wherein the composition comprises a polymer, and the polymer is, or comprises, a poly(ethylene glycol) polymer.

4822. The method of item 4685, wherein the composition comprises a polymer, and the polymer is, or comprises, an amorphous polymer.

4823. The method of item 4685, wherein the composition further comprises a second pharmaceutically active agent.

4824. The method of item 4685, wherein the composition further comprises an anti-inflammatory agent.

4825. The method of item 4685, wherein the composition further comprises an agent that inhibits infection.

4826. The method of item 4685, wherein the composition further comprises an anthracycline.

4827. The method of item 4685, wherein the composition further comprises doxorubicin.

4828. The method of item 4685 wherein the composition further comprises mitoxantrone.

4829. The method of item 4685 wherein the composition further comprises a fluoropyrimidine.

4830. The method of item 4685, wherein the composition further comprises 5-fluorouracil (5-FU).

4831. The method of item 4685, wherein the composition further comprises a folic acid antagonist.

4832. The method of item 4685, wherein the composition further comprises methotrexate.

4833. The method of item 4685, wherein the composition further comprises a podophylotoxin.

4834. The method of item 4685, wherein the composition further comprises etoposide.

4835. The method of item 4685, wherein the composition further comprises camptothecin.

4836. The method of item 4685, wherein the composition further comprises a hydroxyurea.

4837. The method of item 4685, wherein the composition further comprises a platinum complex.

4838. The method of item 4685, wherein the composition further comprises cisplatin.

4839. The method of item 4685 wherein the composition further comprises an anti-thrombotic agent.

4840. The method of item 4685, wherein the composition further comprises a visualization agent

4841. The method of item 4685, wherein the composition further comprises a visualization agent, and the visualization agent is a radiopaque material, wherein the radiopaque material comprises a metal, a halogenated compound, or a barium containing compound.

4842. The method of item 4685, wherein the composition further comprises a visualization agent, and the visualization agent is, or comprises, barium, tantalum, or technetium.

4843. The method of item 4685, wherein the composition further comprises a visualization agent, and the visualization agent is, or comprises, an MRI responsive material.

4844. The method of item 4685, wherein the composition further comprises a visualization agent, and the visualization agent is, or comprises, a gadolinium chelate.

4845. The method of item 4685, wherein the composition further comprises a visualization agent, and the visualization agent is, or comprises, iron, magnesium, manganese, copper, or chromium.

4846. The method of item 4685, wherein the composition further comprises a visualization agent, and the visualization agent is, or comprises, iron oxide compound.

4847. The method of item 4685, wherein the composition further comprises a visualization agent, and the visualization agent is, or comprises, a dye, pigment, or colorant.

4848. The method of item 4685 wherein the agent is released in effective concentrations from the composition comprising the agent by diffusion over a period ranging from the time of administration to about 90 days.

4849. The method of item 4685 wherein the agent is released in effective concentrations from the composition comprising the agent by erosion of the composition over a period ranging from the time of administration to about 90 days.

4850. The method of item 4685 wherein the composition further comprises an inflammatory cytokine.

4851. The method of item 4685 wherein the composition further comprises an agent that stimulates cell proliferation.

4852. The method of item 4685 wherein the composition further comprises a polymeric carrier.

4853. The method of item 4685 wherein the composition is in the form of a gel, paste, or spray.

4854. The method of item 4685 wherein the electrical device is partially constructed with the agent or the composition.

4855. The method of item 4685 wherein the electrical device is impregnated with the agent or the composition.

4856. The method of item 4685, wherein the agent or the composition forms a coating, and the coating directly contacts the electrical device.

4857. The method of item 4685, wherein the agent or the composition forms a coating, and the coating indirectly contacts the electrical device.

4858. The method of item 4685 wherein the agent or the composition forms a coating, and the coating partially covers the electrical device.

4859. The method of item 4685, wherein the agent or the composition forms a coating, and the coating completely covers the electrical device.

4860. The method of item 4685 wherein the agent or the composition is located within pores or holes of the electrical device.

4861. The method of item 4685 wherein the agent or the composition is located within a channel, lumen, or divet of the electrical device.

4862. The method of item 4685 wherein the electrical device further comprises an echogenic material.

4863. The method of item 4685 wherein the electrical device further comprises an echogenic material, wherein the echogenic material is in the form of a coating.

4864. The method of item 4685 wherein the electrical device is sterile.

4865. The method of item 4685 wherein the agent is delivered from the electrical device, wherein the agent is released into tissue in the vicinity of the electrical device after deployment of the electrical device.

4866. The method of item 4685 wherein the agent is delivered from the electrical device, wherein the agent is released into tissue in the vicinity of the electrical device after deployment of the electrical device, wherein the tissue is connective tissue.

4867. The method of item 4685 wherein the agent is delivered from the electrical device, wherein the agent is released into tissue in the vicinity of the electrical device after deployment of the electrical device, wherein the tissue is muscle tissue.

4868. The method of item 4685 wherein the agent is delivered from the electrical device, wherein the agent is released into tissue in the vicinity of the electrical device after deployment of the electrical device, wherein the tissue is nerve tissue.

4869. The method of item 4685 wherein the agent is delivered from the electrical device, wherein the agent is released into tissue in the vicinity of the electrical device after deployment of the electrical device, wherein the tissue is epithelium tissue.

4870. The method of item 4685 wherein the agent is delivered from the electrical device, wherein the agent is released in effective concentrations from the electrical device over a period ranging from the time of deployment of the electrical device to about 1 year.

4871. The method of item 4685 wherein the agent is delivered from the electrical device, wherein the agent is released in effective concentrations from the electrical device over a period ranging from about 1 month to 6 months.

4872. The method of item 4685 wherein the agent is delivered from the electrical device, wherein the agent is released in effective concentrations from the electrical device over a period ranging from about 1-90 days.

4873. The method of item 4685 wherein the agent is delivered from the electrical device, wherein the agent is released in effective concentrations from the electrical device at a constant rate.

4874. The method of item 4685 wherein the agent is delivered from the electrical device, wherein the agent is released in effective concentrations from the electrical device at an increasing rate.

4875. The method of item 4685 wherein the agent is delivered from the electrical device, wherein the agent is released in effective concentrations from the electrical device at a decreasing rate.

4876. The method of item 4685 wherein the agent is delivered from the electrical device, wherein the electrical device comprises about 0.01 μg to about 10 μg of the agent.

4877. The method of item 4685 wherein the agent is delivered from the electrical device, wherein the electrical device comprises about 10 μg to about 10 mg of the agent.

4878. The method of item 4685 wherein the agent is delivered from the electrical device, wherein the electrical device comprises about 10 mg to about 250 mg of the agent.

4879. The method of item 4685 wherein the agent is delivered from the electrical device, wherein the electrical device comprises about 250 mg to about 1000 mg of the agent

4880. The method of item 4685 wherein the agent is delivered from the electrical device, wherein the electrical device comprises about 1000 mg to about 2500 mg of the agent.

4881. The method of item 4685 wherein the agent is delivered from the electrical device, wherein a surface of the electrical device comprises less than 0.01 μg of the agent per mm² of electrical device surface to which the agent is applied.

4882. The method of item 4685 wherein the agent is delivered from the electrical device, wherein a surface of the electrical device comprises about 0.01 μg to about 1 μg of the agent per mm² of electrical device surface to which the agent is applied.

4883. The method of item 4685 wherein the agent is delivered from the electrical device, wherein a surface of the electrical device comprises about 1 μg to about 10 μg of the agent per mm² of electrical device surface to which the agent is applied.

4884. The method of item 4685 wherein the agent is delivered from the electrical device, wherein a surface of the electrical device comprises about 10 μg to about 250 μg of the agent per mm² of electrical device surface to which the agent is applied.

4885. The method of item 4685 wherein the agent is delivered from the electrical device, wherein a surface of the electrical device comprises about 250 μg to about 1000 μg of the agent per mm² of electrical device surface to which the agent is applied.

4886. The method of item 4685 wherein the agent is delivered from the electrical device, wherein a surface of the electrical device comprises about 1000 μg to about 2500 μg of the agent per mm² of electrical device surface to which the agent is applied.

4887. The method of item 4685, wherein the electrical device further comprises a coating, and the coating is a uniform coating.

4888. The method of item 4685, wherein the electrical device further comprises a coating, and the coating is a non-uniform coating.

4889. The method of item 4685, wherein the electrical device further comprises a coating, and the coating is a discontinuous coating.

4890. The method of item 4685, wherein the electrical device further comprises a coating, and the coating is a patterned coating.

4891. The method of item 4685, wherein the electrical device further comprises a coating, and the coating has a thickness of 100 μm or less.

4892. The method of item 4685, wherein the electrical device further comprises a coating, and the coating has a thickness of 10 μm or less.

4893. The method of item 4685, wherein the electrical device further comprises a coating, and the coating adheres to the surface of the electrical device upon deployment of the electrical device.

4894. The method of item 4685, wherein the electrical device further comprises a coating, and the coating is stable at room temperature for a period of at least 1 year.

4895. The method of item 4685, wherein the electrical device further comprises a coating, and the agent is present in the coating in an amount ranging between about 0.0001% to about 1% by weight.

4896. The method of item 4685, wherein the electrical device further comprises a coating, and the agent is present in the coating in an amount ranging between about 1% to about 10% by weight.

4897. The method of item 4685, wherein the electrical device further comprises a coating, and the agent is present in the coating in an amount ranging between about 10% to about 25% by weight.

4898. The method of item 4685, wherein the electrical device further comprises a coating, and the agent is present in the coating in an amount ranging between about 25% to about 70% by weight.

4899. The method of item 4685, wherein the electrical device further comprises a coating, and the coating comprises a polymer.

4900. The method of item 4685, wherein the electrical device comprises a first coating having a first composition and a second coating having a second composition.

4901. The method of item 4685, wherein the electrical device comprises a first coating having a first composition and a second coating having a second composition, wherein the first composition and the second composition are different.

4902. The method of item 4685 wherein the agent or the composition is affixed to the electrical device.

4903. The method of item 4685 wherein the agent or the composition is covalently attached to the electrical device.

4904. The method of item 4685 wherein the agent or the composition is non-covalently attached to the electrical device.

4905. The method of item 4685 wherein the electrical device comprises a coating that absorbs the agent or the composition.

4906. The method of item 4685 wherein the electrical device is interweaved with a thread composed of, or coated with, the agent or the composition.

4907. The method of item 4685 wherein a portion of the electrical device is covered with a sleeve that contains the agent or the composition.

4908. The method of item 4685 wherein the electrical device is completely covered with a sleeve that contains the agent or the composition.

4909. The method of item 4685 wherein a portion of the electrical device is covered with a mesh that contains the agent or the composition.

4910. The method of item 4685 wherein the electrical device is completely covered with a mesh that contains the agent or the composition.

4911. The method of item 4685 wherein the agent or the composition is applied to the electrical device surface prior to the placing of the electrical device into the host.

4912. The method of item 4685 wherein the agent or the composition is applied to the electrical device surface during the placing of the electrical device into the host.

4913. The method of item 4685 wherein the agent or the composition is applied to the electrical device surface immediately after the placing of the electrical device into the host.

4914. The method of item 4685 wherein the agent or the composition is applied to the surface of the tissue in the host surrounding the electrical device prior to the placing of the electrical device into the host.

4915. The method of item 4685 wherein the agent or the composition is applied to the surface of the tissue in the host surrounding the electrical device during the placing of the electrical device into the host.

4916. The method of item 4685 wherein the agent or the composition is applied to the surface of the tissue in the host surrounding the electrical device immediately after the placing of the electrical device into the host.

4917. The method of item 4685 wherein the agent or the composition is topically applied into the anatomical space where the electrical device is placed.

4918. The method of item 4685 wherein the agent or the composition is percutaneously injected into the tissue in the host surrounding the electrical device.

4919. The method for inhibiting scarring of any one of items 4685-4918 wherein the vagal nerve stimulator is adapted for treating or preventing depression.

4920. The method for inhibiting scarring of any one of items 4685-4918 wherein the vagal nerve stimulator is adapted for treating or preventing anxiety.

4921. The method for inhibiting scarring of any one of items 4685-4918 wherein the vagal nerve stimulator is adapted for treating or preventing panic disorders.

4922. The method for inhibiting scarring of any one of items 4685-4918 wherein the vagal nerve stimulator is adapted for treating or preventing obsessive-compulsive disorders.

4923. The method for inhibiting scarring of any one of items 4685-4918 wherein the vagal nerve stimulator is adapted for treating or preventing post-traumatic disorders.

4924. The method for inhibiting scarring of any one of items 4685-4918 wherein the vagal nerve stimulator is adapted for treating or preventing obesity.

4925. The method for inhibiting scarring of any one of items 4685-4918 wherein the vagal nerve stimulator is adapted for treating or preventing migraine.

4926. The method for inhibiting scarring of any one of items 4685-4918 wherein the vagal nerve stimulator is adapted for treating or preventing sleep disorders.

4927. The method for inhibiting scarring of any one of items 4685-4918 wherein the vagal nerve stimulator is adapted for treating or preventing dementia.

4928. The method for inhibiting scarring of any one of items 4685-4918 wherein the vagal nerve stimulator is adapted for treating or preventing Alzheimer's disease.

4929. The method for inhibiting scarring of any one of items 4685-4918 wherein the vagal nerve stimulator is adapted for treating or preventing chronic or degenerative neurological disorders.

4930. A method for inhibiting scarring comprising placing a sacral nerve stimulator for treating a bladder control problem (i.e., an electrical device) and an anti-scarring agent or a composition comprising an ant-scarring agent into an animal host, wherein the agent inhibits scarring.

4931. The method of item 4930 wherein the agent is an adensosine A2A receptor antagonist.

4932. The method of item 4930 wherein the agent is an AKT inhibitor.

4933. The method of item 4930 wherein the agent is an alpha 2 integrin antagonist, wherein the alpha 2 integrin antagonist is Pharmaprojects No. 5754 (Merck KgaA).

4934. The method of item 4930 wherein the agent is an alpha 4 integrin antagonist.

4935. The method of item 4930 wherein the agent is an alpha 7 nicotinic receptor agonist.

4936. The method of item 4930 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

4937. The method of item 4930 wherein the agent is an apoptosis antagonist.

4938. The method of item 4930 wherein the agent is an apoptosis activator.

4939. The method of item 4930 wherein the agent is a beta 1 integrin antagonist.

4940. The method of item 4930 wherein the agent is a beta tubulin inhibitor.

4941. The method of item 4930 wherein the agent is a blocker of enzyme production in Hepatitis C.

4942. The method of item 4930 wherein the agent is a Bruton's tyrosine kinase inhibitor.

4943. The method of item 4930 wherein the agent is a calcineurin inhibitor.

4944. The method of item 4930 wherein the agent is a caspase 3 inhibitor.

4945. The method of item 4930 wherein the agent is a CC chemokine receptor antagonist.

4946. The method of item 4930 wherein the agent is a cell cycle inhibitor selected from the group consisting of SNS-595 (Sunesis), synthadotin, KRX-0403, and an analogue or derivative thereof.

4947. The method of item 4930 wherein the agent is a cathepsin B inhibitor.

4948. The method of item 4930 wherein the agent is a cathepsin K inhibitor, wherein the cathepsin K inhibitor is 462795 (GlaxoSmithKline), INPL-022-D6 (Amura Therapeutics), or an analogue or derivative thereof.

4949. The method of item 4930 wherein the agent is a cathepsin L inhibitor.

4950. The method of item 4930 wherein the agent is a CD40 antagonist.

4951. The method of item 4930 wherein the agent is a chemokine receptor agonist.

4952. The method of item 4930 wherein the agent is a chymase inhibitor.

4953. The method of item 4930 wherein the agent is a collagenase antagonist.

4954. The method of item 4930 wherein the agent is a CXCR antagonist.

4955. The method of item 4930 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

4956. The method of item 4930 wherein the agent is a cyclooxygenase 1 inhibitor.

4957. The method of item 4930 wherein the agent is a DHFR inhibitor.

4958. The method of item 4930 wherein the agent is a dual integrin inhibitor.

4959. The method of item 4930 wherein the agent is an elastase inhibitor.

4960. The method of item 4930 wherein the agent is an elongation factor-1 alpha inhibitor.

4961. The method of item 4930 wherein the agent is an endothelial growth factor antagonist.

4962. The method of item 4930 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

4963. The method of item 4930 wherein the agent is an endotoxin antagonist.

4964. The method of item 4930 wherein the agent is an epothilone and tubulin binder.

4965. The method of item 4930 wherein the agent is an estrogen receptor antagonist.

4966. The method of item 4930 wherein the agent is an FGF inhibitor.

4967. The method of item 4930 wherein the agent is a farnexyl transferase inhibitor.

4968. The method of item 4930 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof

4969. The method of item 4930 wherein the agent is an FLT-3 kinase inhibitor.

4970. The method of item 4930 wherein the agent is an FGF receptor kinase inhibitor.

4971. The method of item 4930 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

4972. The method of item 4930 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

4973. The method of item 4930 wherein the agent is a histone deacetylase inhibitor.

4974. The method of item 4930 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

4975. The method of item 4930 wherein the agent is an ICAM inhibitor.

4976. The method of item 4930 wherein the agent is an IL, ICE and IRAK antagonist, wherein the antagonist is a CJ-14877, CP-424174 (Pfizer), NF-61 (Negma-Lerads), and an analogue or derivative thereof.

4977. The method of item 4930 wherein the agent is an IL-2 inhibitor.

4978. The method of item 4930 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

4979. The method of item 4930 wherein the agent is an IMPDH (inosine monophosphate).

4980. The method of item 4930 wherein the agent is an integrin antagonist.

4981. The method of item 4930 wherein the agent is an interleukin antagonist.

4982. The method of item 4930 wherein the agent is an inhibitor of type III receptor tyrosine kinase.

4983. The method of item 4930 wherein the agent is an irreversible inhibitor of enzyme methionine aminopeptidase type 2.

4984. The method of item 4930 wherein the agent is an isozyme selective delta protein kinase C inhibitor.

4985. The method of item 4930 wherein the agent a JAK3 enzyme inhibitor.

4986. The method of item 4930 wherein the agent is a JNK inhibitor.

4987. The method of item 4930 wherein the agent is a kinase inhibitor.

4988. The method of item 4930 wherein the agent is kinesin antagonist.

4989. The method of item 4930 wherein the agent is a kinesin antagonist.

4990. The method of item 4930 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 14183549-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

4991. The method of item 4930 wherein the agent is an MAP kinase inhibitor.

4992. The method of item 4930 wherein the agent is a matrix metalloproteinase inhibitor.

4993. The method of item 4930 wherein the agent is an MCP-CCR2 inhibitor.

4994. The method of item 4930 wherein the agent is an mTOR inhibitor.

4995. The method of item 4930 wherein the agent is an mTOR kinase inhibitor.

4996. The method of item 4930 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

4997. The method of item 4930 wherein the agent is an MIF inhibitor.

4998. The method of item 4930 wherein the agent is an MMP inhibitor.

4999. The method of item 4930 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

5000. The method of item 4930 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

5001. The method of item 4930 wherein the agent is a nitric oxide agonist.

5002. The method of item 4930 wherein the agent is an ornithine decarboxylase inhibitor.

5003. The method of item 4930 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

5004. The method of item 4930 wherein the agent is a palmitoyl-protein thioesterase inhibitor.

5005. The method of item 4930 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

5006. The method of item 4930 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

5007. The method of item 4930 wherein the agent is a phosphatase inhibitor.

5008. The method of item 4930 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

5009. The method of item 4930 wherein the agent is a PKC inhibitor.

5010. The method of item 4930 wherein the agent is a platelet activating factor antagonist.

5011. The method of item 4930 wherein the agent is a platelet-derived growth factor receptor kinase inhibitor.

5012. The method of item 4930 wherein the agent is a prolyl hydroxylase inhibitor.

5013. The method of item 4930 wherein the agent is a polymorphonuclear neutrophil inhibitor.

5014. The method of item 4930 wherein the agent is a protein kinase B inhibitor.

5015. The method of item 4930 wherein the agent is a protein kinase C stimulant.

5016. The method of item 4930 wherein the agent is a purine nucleoside analogue.

5017. The method of item 4930 wherein the agent is a purinoreceptor P2X antagonist.

5018. The method of item 4930 wherein the agent is a Raf kinase inhibitor.

5019. The method of item 4930 wherein the agent is a reversible inhibitor of ErbB1 and ErbB2.

5020. The method of item 4930 wherein the agent is a ribonucleoside triphosphate reductase inhibitor.

5021. The method of item 4930 wherein the agent is an SDF-1 antagonist.

5022. The method of item 4930 wherein the agent is a sheddase inhibitor.

5023. The method of item 4930 wherein the agent is an SRC inhibitor.

5024. The method of item 4930 wherein the agent is a stromelysin inhibitor.

5025. The method of item 4930 wherein the agent is an Syk kinase inhibitor.

5026. The method of item 4930 wherein the agent is a telomerase inhibitor.

5027. The method of item 4930 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

5028. The method of item 4930 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

5029. The method of item 4930 wherein the agent is a Toll receptor inhibitor.

5030. The method of item 4930 wherein the agent is a tubulin antagonist.

5031. The method of item 4930 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-11 0 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

5032. The method of item 4930 wherein the agent is a VEGF inhibitor.

5033. The method of item 4930 wherein the agent is a vitamin D receptor agonist.

5034. The method of item 4930 wherein the agent is ZD-6474 (an angiogenesis inhibitor).

5035. The method of item 4930 wherein the agent is AP-23573 (an mTOR inhibitor).

5036. The method of item 4930 wherein the agent is synthadotin (a tubulin antagonist).

5037. The method of item 4930 wherein the agent is S-0885 (a collagenase inhibitor).

5038. The method of item 4930 wherein the agent is aplidine (an elongation factor-1 alpha inhibitor).

5039. The method of item 4930 wherein the agent is ixabepilone (an epithilone)

5040. The method of item 4930 wherein the agent is IDN-5390 (an angiogenesis inhibitor).

5041. The method of item 4930 wherein the agent is SB-2723005 (an angiogenesis inhibitor).

5042. The method of item 4930 wherein the agent is ABT-518 (an angiogenesis inhibitor).

5043. The method of item 4930 wherein the agent is combretastatin (an angiogenesis inhibitor).

5044. The method of item 4930 wherein the agent is anecortave acetate (an angiogenesis inhibitor).

5045. The method of item 4930 wherein the agent is SB-715992 (a kinesin antagonist).

5046. The method of item 4930 wherein the agent is temsirolimus (an mTOR inhibitor).

5047. The method of item 4930 wherein the agent is adalimumab (a TNFα antagonist).

5048. The method of item 4930, wherein the composition comprises a polymer.

5049. The method of item 4930, wherein the composition comprises a polymer, and the polymer is, or comprises, a copolymer.

5050. The method of item 4930, wherein the composition comprises a polymer, and the polymer is, or comprises, a block copolymer.

5051. The method of item 4930, wherein the composition comprises a polymer, and the polymer is, or comprises, a random copolymer.

5052. The method of item 4930, wherein the composition comprises a polymer, and the polymer is, or comprises, a biodegradable polymer.

5053. The method of item 4930, wherein the composition comprises a polymer, and the polymer is, or comprises, a non-biodegradable polymer.

5054. The method of item 4930, wherein the composition comprises a polymer, and the polymer is, or comprises, a hydrophilic polymer.

5055. The method of item 4930, wherein the composition comprises a polymer, and the polymer is, or comprises, a hydrophobic polymer.

5056. The method of item 4930, wherein the composition comprises a polymer, and the polymer is, or comprises, a polymer having hydrophilic domains.

5057. The method of item 4930, wherein the composition comprises a polymer, and the polymer is, or comprises, a polymer having hydrophobic domains.

5058. The method of item 4930, wherein the composition comprises a polymer, and the polymer is, or comprises, a non-conductive polymer.

5059. The method of item 4930, wherein the composition comprises a polymer, and the polymer is, or comprises, an elastomer.

5060. The method of item 4930, wherein the composition comprises a polymer, and the polymer is, or comprises, a hydrogel.

5061. The method of item 4930, wherein the composition comprises a polymer, and the polymer is, or comprises, a silicone polymer.

5062. The method of item 4930, wherein the composition comprises a polymer, and the polymer is, or comprises, a hydrocarbon polymer.

5063. The method of item 4930, wherein the composition comprises a polymer, and the polymer is, or comprises, a styrene-derived polymer.

5064. The method of item 4930, wherein the composition comprises a polymer, and the polymer is, or comprises, a butadiene-derived polymer.

5065. The method of item 4930, wherein the composition comprises a polymer, and the polymer is, or comprises, a macromer.

5066. The method of item 4930, wherein the composition comprises a polymer, and the polymer is, or comprises, a poly(ethylene glycol) polymer.

5067. The method of item 4930, wherein the composition comprises a polymer, and the polymer is, or comprises, an amorphous polymer.

5068. The method of item 4930, wherein the composition further comprises a second pharmaceutically active agent.

5069. The method of item 4930, wherein the composition further comprises an anti-inflammatory agent.

5070. The method of item 4930, wherein the composition further comprises an agent that inhibits infection.

5071. The method of item 4930, wherein the composition further comprises an anthracycline.

5072. The method of item 4930, wherein the composition further comprises doxorubicin.

5073. The method of item 4930 wherein the composition further comprises mitoxantrone.

5074. The method of item 4930 wherein the composition further comprises a fluoropyrimidine.

5075. The method of item 4930, wherein the composition further comprises 5-fluorouracil (5-FU).

5076. The method of item 4930, wherein the composition further comprises a folic acid antagonist.

5077. The method of item 4930, wherein the composition further comprises methotrexate.

5078. The method of item 4930, wherein the composition further comprises a podophylotoxin.

5079. The method of item 4930, wherein the composition further comprises etoposide.

5080. The method of item 4930, wherein the composition further comprises camptothecin.

5081. The method of item 4930, wherein the composition further comprises a hydroxyurea.

5082. The method of item 4930, wherein the composition further comprises a platinum complex.

5083. The method of item 4930, wherein the composition further comprises cisplatin.

5084. The method of item 4930 wherein the composition further comprises an anti-thrombotic agent.

5085. The method of item 4930, wherein the composition further comprises a visualization agent.

5086. The method of item 4930, wherein the composition further comprises a visualization agent, and the visualization agent is a radiopaque material, wherein the radiopaque material comprises a metal, a halogenated compound, or a barium containing compound.

5087. The method of item 4930, wherein the composition further comprises a visualization agent, and the visualization agent is, or comprises, barium, tantalum, or technetium.

5088. The method of item 4930, wherein the composition further comprises a visualization agent, and the visualization agent is, or comprises, an MRI responsive material.

5089. The method of item 4930, wherein the composition further comprises a visualization agent, and the visualization agent is, or comprises, a gadolinium chelate.

5090. The method of item 4930, wherein the composition further comprises a visualization agent, and the visualization agent is, or comprises, iron, magnesium, manganese, copper, or chromium.

5091. The method of item 4930, wherein the composition further comprises a visualization agent, and the visualization agent is, or comprises, iron oxide compound.

5092. The method of item 4930, wherein the composition further comprises a visualization agent, and the visualization agent is, or comprises, a dye, pigment, or colorant.

5093. The method of item 4930 wherein the agent is released in effective concentrations from the composition comprising the agent by diffusion over a period ranging from the time of administration to about 90 days.

5094. The method of item 4930 wherein the agent is released in effective concentrations from the composition comprising the agent by erosion of the composition over a period ranging from the time of administration to about 90 days.

5095. The method of item 4930 wherein the composition further comprises an inflammatory cytokine.

5096. The method of item 4930 wherein the composition further comprises an agent that stimulates cell proliferation.

5097. The method of item 4930 wherein the composition further comprises a polymeric carrier.

5098. The method of item 4930 wherein the composition is in the form of a gel, paste, or spray.

5099. The method of item 4930 wherein the electrical device is partially constructed with the agent or the composition.

5100. The method of item 4930 wherein the electrical device is impregnated with the agent or the composition.

5101. The method of item 4930, wherein the agent or the composition forms a coating, and the coating directly contacts the electrical device.

5102. The method of item 4930, wherein the agent or the composition forms a coating, and the coating indirectly contacts the electrical device.

5103. The method of item 4930 wherein the agent or the composition forms a coating, and the coating partially covers the electrical device.

5104. The method of item 4930, wherein the agent or the composition forms a coating, and the coating completely covers the electrical device.

5105. The method of item 4930 wherein the agent or the composition is located within pores or holes of the electrical device.

5106. The method of item 4930 wherein the agent or the composition is located within a channel, lumen, or divet of the electrical device.

5107. The method of item 4930 wherein the electrical device further comprises an echogenic material.

5108. The method of item 4930 wherein the electrical device further comprises an echogenic material, wherein the echogenic material is in the form of a coating.

5109. The method of item 4930 wherein the electrical device is sterile.

5110. The method of item 4930 wherein the agent is delivered from the electrical device, wherein the agent is released into tissue in the vicinity of the electrical device after deployment of the electrical device.

5111. The method of item 4930 wherein the agent is delivered from the electrical device, wherein the agent is released into tissue in the vicinity of the electrical device after deployment of the electrical device, wherein the tissue is connective tissue.

5112. The method of item 4930 wherein the agent is delivered from the electrical device, wherein the agent is released into tissue in the vicinity of the electrical device after deployment of the electrical device, wherein the tissue is muscle tissue.

5113. The method of item 4930 wherein the agent is delivered from the electrical device, wherein the agent is released into tissue in the vicinity of the electrical device after deployment of the electrical device, wherein the tissue is nerve tissue.

5114. The method of item 4930 wherein the agent is delivered from the electrical device, wherein the agent is released into tissue in the vicinity of the electrical device after deployment of the electrical device, wherein the tissue is epithelium tissue.

5115. The method of item 4930 wherein the agent is delivered from the electrical device, wherein the agent is released in effective concentrations from the electrical device over a period ranging from the time of deployment of the electrical device to about 1 year.

5116. The method of item 4930 wherein the agent is delivered from the electrical device, wherein the agent is released in effective concentrations from the electrical device over a period ranging from about 1 month to 6 months.

5117. The method of item 4930 wherein the agent is delivered from the electrical device, wherein the agent is released in effective concentrations from the electrical device over a period ranging from about 1-90 days.

5118. The method of item 4930 wherein the agent is delivered from the electrical device, wherein the agent is released in effective concentrations from the electrical device at a constant rate.

5119. The method of item 4930 wherein the agent is delivered from the electrical device, wherein the agent is released in effective concentrations from the electrical device at an increasing rate.

5120. The method of item 4930 wherein the agent is delivered from the electrical device, wherein the agent is released in effective concentrations from the electrical device at a decreasing rate.

5121. The method of item 4930 wherein the agent is delivered from the electrical device, wherein the electrical device comprises about 0.01 μg to about 10 μg of the agent.

5122. The method of item 4930 wherein the agent is delivered from the electrical device, wherein the electrical device comprises about 10 μg to about 10 mg of the agent.

5123. The method of item 4930 wherein the agent is delivered from the electrical device, wherein the electrical device comprises about 10 mg to about 250 mg of the agent.

5124. The method of item 4930 wherein the agent is delivered from the electrical device, wherein the electrical device comprises about 250 mg to about 1000 mg of the agent.

5125. The method of item 4930 wherein the agent is delivered from the electrical device, wherein the electrical device comprises about 1000 mg to about 2500 mg of the agent.

5126. The method of item 4930 wherein the agent is delivered from the electrical device, wherein a surface of the electrical device comprises less than 0.01 μg of the agent per mm² of electrical device surface to which the agent is applied.

5127. The method of item 4930 wherein the agent is delivered from the electrical device, wherein a surface of the electrical device comprises about 0.01 μg to about 1 μg of the agent per mm² of electrical device surface to which the agent is applied.

5128. The method of item 4930 wherein the agent is delivered from the electrical device, wherein a surface of the electrical device comprises about 1 μg to about 10 μg of the agent per mm² of electrical device surface to which the agent is applied.

5129. The method of item 4930 wherein the agent is delivered from the electrical device, wherein a surface of the electrical device comprises about 10 μg to about 250 μg of the agent per mm² of electrical device surface to which the agent is applied.

5130. The method of item 4930 wherein the agent is delivered from the electrical device, wherein a surface of the electrical device comprises about 250 μg to about 1000 μg of the agent per mm² of electrical device surface to which the agent is applied.

5131. The method of item 4930 wherein the agent is delivered from the electrical device, wherein a surface of the electrical device comprises about 1000 μg to about 2500 μg of the agent per mm² of electrical device surface to which the agent is applied.

5132. The method of item 4930, wherein the electrical device further comprises a coating, and the coating is a uniform coating.

5133. The method of item 4930, wherein the electrical device further comprises a coating, and the coating is a non-uniform coating.

5134. The method of item 4930, wherein the electrical device further comprises a coating, and the coating is a discontinuous coating.

5135. The method of item 4930, wherein the electrical device further comprises a coating, and the coating is a patterned coating.

5136. The method of item 4930, wherein the electrical device further comprises a coating, and the coating has a thickness of 100 μm or less.

5137. The method of item 4930, wherein the electrical device further comprises a coating, and the coating has a thickness of 10 μm or less.

5138. The method of item 4930, wherein the electrical device further comprises a coating, and the coating adheres to the surface of the electrical device upon deployment of the electrical device.

5139. The method of item 4930, wherein the electrical device further comprises a coating, and the coating is stable at room temperature for a period of at least 1 year.

5140. The method of item 4930, wherein the electrical device further comprises a coating, and the agent is present in the coating in an amount ranging between about 0.0001% to about 1% by weight.

5141. The method of item 4930, wherein the electrical device further comprises a coating, and the agent is present in the coating in an amount ranging between about 1% to about 10% by weight.

5142. The method of item 4930, wherein the electrical device further comprises a coating, and the agent is present in the coating in an amount ranging between about 10% to about 25% by weight.

5143. The method of item 4930, wherein the electrical device further comprises a coating, and the agent is present in the coating in an amount ranging between about 25% to about 70% by weight.

5144. The method of item 4930, wherein the electrical device further comprises a coating, and the coating comprises a polymer.

5145. The method of item 4930, wherein the electrical device comprises a first coating having a first composition and a second coating having a second composition.

5146. The method of item 4930, wherein the electrical device comprises a first coating having a first composition and a second coating having a second composition, wherein the first composition and the second composition are different.

5147. The method of item 4930 wherein the agent or the composition is affixed to the electrical device.

5148. The method of item 4930 wherein the agent or the composition is covalently attached to the electrical device.

5149. The method of item 4930 wherein the agent or the composition is non-covalently attached to the electrical device.

5150. The method of item 4930 wherein the electrical device comprises a coating that absorbs the agent or the composition.

5151. The method of item 4930 wherein the electrical device is interweaved with a thread composed of, or coated with, the agent or the composition.

5152. The method of item 4930 wherein a portion of the electrical device is covered with a sleeve that contains the agent or the composition.

5153. The method of item 4930 wherein the electrical device is completely covered with a sleeve that contains the agent or the composition.

5154. The method of item 4930 wherein a portion of the electrical device is covered with a mesh that contains the agent or the composition.

5155. The method of item 4930 wherein the electrical device is completely covered with a mesh that contains the agent or the composition.

5156. The method of item 4930 wherein the agent or the composition is applied to the electrical device surface prior to the placing of the electrical device into the host.

5157. The method of item 4930 wherein the agent or the composition is applied to the electrical device surface during the placing of the electrical device into the host.

5158. The method of item 4930 wherein the agent or the composition is applied to the electrical device surface immediately after the placing of the electrical device into the host.

5159. The method of item 4930 wherein the agent or the composition is applied to the surface of the tissue in the host surrounding the electrical device prior to the placing of the electrical device into the host.

5160. The method of item 4930 wherein the agent or the composition is applied to the surface of the tissue in the host surrounding the electrical device during the placing of the electrical device into the host.

5161. The method of item 4930 wherein the agent or the composition is applied to the surface of the tissue in the host surrounding the electrical device immediately after the placing of the electrical device into the host.

5162. The method of item 4930 wherein the agent or the composition is topically applied into the anatomical space where the electrical device is placed.

5163. The method of item 4930 wherein the agent or the composition is percutaneously injected into the tissue in the host surrounding the electrical device.

5164. The method for inhibiting scarring of any one of items 4930-5163 wherein the sacral nerve stimulator is adapted for treating or preventing urge incontinence.

5165. The method for inhibiting scarring of any one of items 4930-5163 wherein the sacral nerve stimulator is adapted for treating or preventing nonobstructive urinary retention.

5166. The method for inhibiting scarring of any one of items 4930-5163 wherein the sacral nerve stimulator is adapted for treating or preventing urgency frequency.

5167. The method for inhibiting scarring of any one of items 4930-5163 wherein the sacral nerve stimulator is an intramuscular electrical stimulator.

5168. The method for inhibiting scarring of any one of items 4930-5163 wherein the sacral nerve stimulator is a leadless, tubular-shaped microstimulator.

5169. A method for inhibiting scarring comprising placing a gastric nerve stimulator for treating a gastrointestinal disorder (i.e., an electrical device) and an anti-scarring agent or a composition comprising an ant-scarring agent into an animal host, wherein the agent inhibits scarring.

5170. The method of item 5169 wherein the agent is an adensosine A2A receptor antagonist.

5171. The method of item 5169 wherein the agent is an AKT inhibitor.

5172. The method of item 5169 wherein the agent is an alpha 2 integrin antagonist, wherein the alpha 2 integrin antagonist is Pharmaprojects No. 5754 (Merck KgaA).

5173. The method of item 5169 wherein the agent is an alpha 4 integrin antagonist.

5174. The method of item 5169 wherein the agent is an alpha 7 nicotinic receptor agonist.

5175. The method of item 5169 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

5176. The method of item 5169 wherein the agent is an apoptosis antagonist.

5177. The method of item 5169 wherein the agent is an apoptosis activator.

5178. The method of item 5169 wherein the agent is a beta 1 integrin antagonist.

5179. The method of item 5169 wherein the agent is a beta tubulin inhibitor.

5180. The method of item 5169 wherein the agent is a blocker of enzyme production in Hepatitis C.

5181. The method of item 5169 wherein the agent is a Bruton's tyrosine kinase inhibitor.

5182. The method of item 5169 wherein the agent is a calcineurin inhibitor.

5183. The method of item 5169 wherein the agent is a caspase 3 inhibitor.

5184. The method of item 5169 wherein the agent is a CC chemokine receptor antagonist.

5185. The method of item 5169 wherein the agent is a cell cycle inhibitor selected from the group consisting of SNS-595 (Sunesis), synthadotin, KRX-0403, and an analogue or derivative thereof.

5186. The method of item 5169 wherein the agent is a cathepsin B inhibitor.

5187. The method of item 5169 wherein the agent is a cathepsin K inhibitor, wherein the cathepsin K inhibitor is 462795 (GlaxoSmithKline), INPL-022-D6 (Amura Therapeutics), or an analogue or derivative thereof.

5188. The method of item 5169 wherein the agent is a cathepsin L inhibitor.

5189. The method of item 5169 wherein the agent is a CD40 antagonist.

5190. The method of item 5169 wherein the agent is a chemokine receptor agonist.

5191. The method of item 5169 wherein the agent is a chymase inhibitor.

5192. The method of item 5169 wherein the agent is a collagenase antagonist.

5193. The method of item 5169 wherein the agent is a CXCR antagonist.

5194. The method of item 5169 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAKI inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

5195. The method of item 5169 wherein the agent is a cyclooxygenase 1 inhibitor.

5196. The method of item 5169 wherein the agent is a DHFR inhibitor.

5197. The method of item 5169 wherein the agent is a dual integrin inhibitor.

5198. The method of item 5169 wherein the agent is an elastase inhibitor.

5199. The method of item 5169 wherein the agent is an elongation factor-1 alpha inhibitor.

5200. The method of item 5169 wherein the agent is an endothelial growth factor antagonist.

5201. The method of item 5169 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

5202. The method of item 5169 wherein the agent is an endotoxin antagonist.

5203. The method of item 5169 wherein the agent is an epothilone and tubulin binder.

5204. The method of item 5169 wherein the agent is an estrogen receptor antagonist.

5205. The method of item 5169 wherein the agent is an FGF inhibitor.

5206. The method of item 5169 wherein the agent is a farnexyl transferase inhibitor.

5207. The method of item 5169 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

5208. The method of item 5169 wherein the agent is an FLT-3 kinase inhibitor.

5209. The method of item 5169 wherein the agent is an FGF receptor kinase inhibitor.

5210. The method of item 5169 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

5211. The method of item 5169 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

5212. The method of item 5169 wherein the agent is a histone deacetylase inhibitor.

5213. The method of item 5169 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

5214. The method of item 5169 wherein the agent is an ICAM inhibitor.

5215. The method of item 5169 wherein the agent is an IL, ICE and IRAK antagonist, wherein the antagonist is a CJ-14877, CP-424174 (Pfizer), NF-61 (Negma-Lerads), and an analogue or derivative thereof.

5216. The method of item 5169 wherein the agent is an IL-2 inhibitor.

5217. The method of item 5169 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

5218. The method of item 5169 wherein the agent is an IMPDH (inosine monophosphate).

5219. The method of item 5169 wherein the agent is an integrin antagonist.

5220. The method of item 5169 wherein the agent is an interleukin antagonist.

5221. The method of item 5169 wherein the agent is an inhibitor of type III receptor tyrosine kinase.

5222. The method of item 5169 wherein the agent is an irreversible inhibitor of enzyme methionine aminopeptidase type 2.

5223. The method of item 5169 wherein the agent is an isozyme selective delta protein kinase C inhibitor.

5224. The method of item 5169 wherein the agent a JAK3 enzyme inhibitor.

5225. The method of item 5169 wherein the agent is a JNK inhibitor.

5226. The method of item 5169 wherein the agent is a kinase inhibitor.

5227. The method of item 5169 wherein the agent is kinesin antagonist.

5228. The method of item 5169 wherein the agent is a kinesin antagonist.

5229. The method of item 5169 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

5230. The method of item 5169 wherein the agent is an MAP kinase inhibitor.

5231. The method of item 5169 wherein the agent is a matrix metalloproteinase inhibitor.

5232. The method of item 5169 wherein the agent is an MCP-CCR2 inhibitor.

5233. The method of item 5169 wherein the agent is an mTOR inhibitor.

5234. The method of item 5169 wherein the agent is an mTOR kinase inhibitor.

5235. The method of item 5169 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

5236. The method of item 5169 wherein the agent is an MIF inhibitor.

5237. The method of item 5169 wherein the agent is an MMP inhibitor.

5238. The method of item 5169 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (GAS No. 334476-64-1) (GlaxoSmithKline), Win-64821 (Sanofi-Aventis), PRX-96026 (Predix Pharmaceuticals), and an analogue or derivative thereof.

5239. The method of item 5169 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

5240. The method of item 5169 wherein the agent is a nitric oxide agonist.

5241. The method of item 5169 wherein the agent is an ornithine decarboxylase inhibitor.

5242. The method of item 5169 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

5243. The method of item 5169 wherein the agent is a palmitoyl-protein thioesterase inhibitor.

5244. The method of item 5169 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof

5245. The method of item 5169 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

5246. The method of item 5169 wherein the agent is a phosphatase inhibitor.

5247. The method of item 5169 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

5248. The method of item 5169 wherein the agent is a PKC inhibitor.

5249. The method of item 5169 wherein the agent is a platelet activating factor antagonist.

5250. The method of item 5169 wherein the agent is a platelet-derived growth factor receptor kinase inhibitor.

5251. The method of item 5169 wherein the agent is a prolyl hydroxylase inhibitor.

5252. The method of item 5169 wherein the agent is a polymorphonuclear neutrophil inhibitor.

5253. The method of item 5169 wherein the agent is a protein kinase B inhibitor.

5254. The method of item 5169 wherein the agent is a protein kinase C stimulant.

5255. The method of item 5169 wherein the agent is a purine nucleoside analogue.

5256. The method of item 5169 wherein the agent is a purinoreceptor P2X antagonist.

5257. The method of item 5169 wherein the agent is a Raf kinase inhibitor.

5258. The method of item 5169 wherein the agent is a reversible inhibitor of ErbB1 and ErbB2.

5259. The method of item 5169 wherein the agent is a ribonucleoside triphosphate reductase inhibitor.

5260. The method of item 5169 wherein the agent is an SDF-1 antagonist.

5261. The method of item 5169 wherein the agent is a sheddase inhibitor.

5262. The method of item 5169 wherein the agent is an SRC inhibitor.

5263. The method of item 5169 wherein the agent is a stromelysin inhibitor.

5264. The method of item 5169 wherein the agent is an Syk kinase inhibitor.

5265. The method of item 5169 wherein the agent is a telomerase inhibitor.

5266. The method of item 5169 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

5267. The method of item 5169 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

5268. The method of item 5169 wherein the agent is a Toll receptor inhibitor.

5269. The method of item 5169 wherein the agent is a tubulin antagonist.

5270. The method of item 5169 wherein the agent is a tyrosine kinase inhibitor selected from the group consisting of SU-011248, SUTENT from Pfizer Inc (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

5271. The method of item 5169 wherein the agent is a VEGF inhibitor.

5272. The method of item 5169 wherein the agent is a vitamin D receptor agonist.

5273. The method of item 5169 wherein the agent is ZD-6474 (an angiogenesis inhibitor).

5274. The method of item 5169 wherein the agent is AP-23573 (an mTOR inhibitor).

5275. The method of item 5169 wherein the agent is synthadotin (a tubulin antagonist).

5276. The method of item 5169 wherein the agent is S-0885 (a collagenase inhibitor).

5277. The method of item 5169 wherein the agent is aplidine (an elongation factor-1 alpha inhibitor).

5278. The method of item 5169 wherein the agent is ixabepilone (an epithilone).

5279. The method of item 5169 wherein the agent is IDN-5390 (an angiogenesis inhibitor).

5280. The method of item 5169 wherein the agent is SB-2723005 (an angiogenesis inhibitor).

5281. The method of item 5169 wherein the agent is ABT-518 (an angiogenesis inhibitor).

5282. The method of item 5169 wherein the agent is combretastatin (an angiogenesis inhibitor).

5283. The method of item 5169 wherein the agent is anecortave acetate (an angiogenesis inhibitor).

5284. The method of item 5169 wherein the agent is SB-715992 (a kinesin antagonist)

5285. The method of item 5169 wherein the agent is temsirolimus (an mTOR inhibitor).

5286. The method of item 5169 wherein the agent is adalimumab (a TNFα antagonist).

5287. The method of item 5169, wherein the composition comprises a polymer.

5288. The method of item 5169, wherein the composition comprises a polymer, and the polymer is, or comprises, a copolymer.

5289. The method of item 5169, wherein the composition comprises a polymer, and the polymer is, or comprises, a block copolymer.

5290. The method of item 5169, wherein the composition comprises a polymer, and the polymer is, or comprises, a random copolymer.

5291. The method of item 5169, wherein the composition comprises a polymer, and the polymer is, or comprises, a biodegradable polymer.

5292. The method of item 5169, wherein the composition comprises a polymer, and the polymer is, or comprises, a non-biodegradable polymer.

5293. The method of item 5169, wherein the composition comprises a polymer, and the polymer is, or comprises, a hydrophilic polymer.

5294. The method of item 5169, wherein the composition comprises a polymer, and the polymer is, or comprises, a hydrophobic polymer.

5295. The method of item 5169, wherein the composition comprises a polymer, and the polymer is, or comprises, a polymer having hydrophilic domains.

5296. The method of item 5169, wherein the composition comprises a polymer, and the polymer is, or comprises, a polymer having hydrophobic domains.

5297. The method of item 5169, wherein the composition comprises a polymer, and the polymer is, or comprises, a non-conductive polymer.

5298. The method of item 5169, wherein the composition comprises a polymer, and the polymer is, or comprises, an elastomer.

5299. The method of item 5169, wherein the composition comprises a polymer, and the polymer is, or comprises, a hydrogel.

5300. The method of item 5169, wherein the composition comprises a polymer, and the polymer is, or comprises, a silicone polymer.

5301. The method of item 5169, wherein the composition comprises a polymer, and the polymer is, or comprises, a hydrocarbon polymer.

5302. The method of item 5169, wherein the composition comprises a polymer, and the polymer is, or comprises, a styrene-derived polymer.

5303. The method of item 5169, wherein the composition comprises a polymer, and the polymer is, or comprises, a butadiene-derived polymer.

5304. The method of item 5169, wherein the composition comprises a polymer, and the polymer is, or comprises, a macromer.

5305. The method of item 5169, wherein the composition comprises a polymer, and the polymer is, or comprises, a poly(ethylene glycol) polymer.

5306. The method of item 5169, wherein the composition comprises a polymer, and the polymer is, or comprises, an amorphous polymer.

5307. The method of item 5169, wherein the composition further comprises a second pharmaceutically active agent.

5308. The method of item 5169, wherein the composition further comprises an anti-inflammatory agent.

5309. The method of item 5169, wherein the composition further comprises an agent that inhibits infection.

5310. The method of item 5169, wherein the composition further comprises an anthracycline.

5311. The method of item 5169, wherein the composition further comprises doxorubicin.

5312. The method of item 5169 wherein the composition further comprises mitoxantrone.

5313. The method of item 5169 wherein the composition further comprises a fluoropyrimidine.

5314. The method of item 5169, wherein the composition further comprises 5-fluorouracil (5-FU).

5315. The method of item 5169, wherein the composition further comprises a folic acid antagonist.

5316. The method of item 5169, wherein the composition further comprises methotrexate.

5317. The method of item 5169, wherein the composition further comprises a podophylotoxin.

5318. The method of item 5169, wherein the composition further comprises etoposide.

5319. The method of item 5169, wherein the composition further comprises camptothecin.

5320. The method of item 5169, wherein the composition further comprises a hydroxyurea.

5321. The method of item 5169, wherein the composition further comprises a platinum complex.

5322. The method of item 5169, wherein the composition further comprises cisplatin.

5323. The method of item 5169 wherein the composition further comprises an anti-thrombotic agent.

5324. The method of item 5169, wherein the composition further comprises a visualization agent.

5325. The method of item 5169, wherein the composition further comprises a visualization agent, and the visualization agent is a radiopaque material, wherein the radiopaque material comprises a metal, a halogenated compound, or a barium containing compound.

5326. The method of item 5169, wherein the composition further comprises a visualization agent, and the visualization agent is, or comprises, barium, tantalum, or technetium.

5327. The method of item 5169, wherein the composition further comprises a visualization agent, and the visualization agent is, or comprises, an MRI responsive material.

5328. The method of item 5169, wherein the composition further comprises a visualization agent, and the visualization agent is, or comprises, a gadolinium chelate.

5329. The method of item 5169, wherein the composition further comprises a visualization agent, and the visualization agent is, or comprises, iron, magnesium, manganese, copper, or chromium.

5330. The method of item 5169, wherein the composition further comprises a visualization agent, and the visualization agent is, or comprises, iron oxide compound.

5331. The method of item 5169, wherein the composition further comprises a visualization agent, and the visualization agent is, or comprises, a dye, pigment, or colorant.

5332. The method of item 5169 wherein the agent is released in effective concentrations from the composition comprising the agent by diffusion over a period ranging from the time of administration to about 90 days.

5333. The method of item 5169 wherein the agent is released in effective concentrations from the composition comprising the agent by erosion of the composition over a period ranging from the time of administration to about 90 days.

5334. The method of item 5169 wherein the composition further comprises an inflammatory cytokine.

5335. The method of item 5169 wherein the composition further comprises an agent that stimulates cell proliferation.

5336. The method of item 5169 wherein the composition further comprises a polymeric carrier.

5337. The method of item 5169 wherein the composition is in the form of a gel, paste, or spray.

5338. The method of item 5169 wherein the electrical device is partially constructed with the agent or the composition.

5339. The method of item 5169 wherein the electrical device is impregnated with the agent or the composition.

5340. The method of item 5169, wherein the agent or the composition forms a coating, and the coating directly contacts the electrical device.

5341. The method of item 5169, wherein the agent or the composition forms a coating, and the coating indirectly contacts the electrical device.

5342. The method of item 5169 wherein the agent or the composition forms a coating, and the coating partially covers the electrical device.

5343. The method of item 5169, wherein the agent or the composition forms a coating, and the coating completely covers the electrical device.

5344. The method of item 5169 wherein the agent or the composition is located within pores or holes of the electrical device.

5345. The method of item 5169 wherein the agent or the composition is located within a channel, lumen, or divet of the electrical device.

5346. The method of item 5169 wherein the electrical device further comprises an echogenic material.

5347. The method of item 5169 wherein the electrical device further comprises an echogenic material, wherein the echogenic material is in the form of a coating.

5348. The method of item 5169 wherein the electrical device is sterile.

5349. The method of item 5169 wherein the agent is delivered from the electrical device, wherein the agent is released into tissue in the vicinity of the electrical device after deployment of the electrical device.

5350. The method of item 5169 wherein the agent is delivered from the electrical device, wherein the agent is released into tissue in the vicinity of the electrical device after deployment of the electrical device, wherein the tissue is connective tissue.

5351. The method of item 5169 wherein the agent is delivered from the electrical device, wherein the agent is released into tissue in the vicinity of the electrical device after deployment of the electrical device, wherein the tissue is muscle tissue.

5352. The method of item 5169 wherein the agent is delivered from the electrical device, wherein the agent is released into tissue in the vicinity of the electrical device after deployment of the electrical device, wherein the tissue is nerve tissue.

5353. The method of item 5169 wherein the agent is delivered from the electrical device, wherein the agent is released into tissue in the vicinity of the electrical device after deployment of the electrical device, wherein the tissue is epithelium tissue.

5354. The method of item 5169 wherein the agent is delivered from the electrical device, wherein the agent is released in effective concentrations from the electrical device over a period ranging from the time of deployment of the electrical device to about 1 year.

5355. The method of item 5169 wherein the agent is delivered from the electrical device, wherein the agent is released in effective concentrations from the electrical device over a period ranging from about 1 month to 6 months.

5356. The method of item 5169 wherein the agent is delivered from the electrical device, wherein the agent is released in effective concentrations from the electrical device over a period ranging from about 1-90 days.

5357. The method of item 5169 wherein the agent is delivered from the electrical device, wherein the agent is released in effective concentrations from the electrical device at a constant rate.

5358. The method of item 5169 wherein the agent is delivered from the electrical device, wherein the agent is released in effective concentrations from the electrical device at an increasing rate.

5359. The method of item 5169 wherein the agent is delivered from the electrical device, wherein the agent is released in effective concentrations from the electrical device at a decreasing rate.

5360. The method of item 5169 wherein the agent is delivered from the electrical device, wherein the electrical device comprises about 0.01 μg to about 10 μg of the agent.

5361. The method of item 5169 wherein the agent is delivered from the electrical device, wherein the electrical device comprises about 10 μg to about 10 mg of the agent.

5362. The method of item 5169 wherein the agent is delivered from the electrical device, wherein the electrical device comprises about 10 mg to about 250 mg of the agent.

5363. The method of item 5169 wherein the agent is delivered from the electrical device, wherein the electrical device comprises about 250 mg to about 1000 mg of the agent.

5364. The method of item 5169 wherein the agent is delivered from the electrical device, wherein the electrical device comprises about 1000 mg to about 2500 mg of the agent.

5365. The method of item 5169 wherein the agent is delivered from the electrical device, wherein a surface of the electrical device comprises less than 0.01 μg of the agent per mm² of electrical device surface to which the agent is applied.

5366. The method of item 5169 wherein the agent is delivered from the electrical device, wherein a surface of the electrical device comprises about 0.01 μg to about 1 μg of the agent per mm² of electrical device surface to which the agent is applied.

5367. The method of item 5169 wherein the agent is delivered from the electrical device, wherein a surface of the electrical device comprises about 1 μg to about 10 μg of the agent per mm² of electrical device surface to which the agent is applied.

5368. The method of item 5169 wherein the agent is delivered from the electrical device, wherein a surface of the electrical device comprises about 10 μg to about 250 μg of the agent per mm² of electrical device surface to which the agent is applied

5369. The method of item 5169 wherein the agent is delivered from the electrical device, wherein a surface of the electrical device comprises about 250 μg to about 1000 μg of the agent per mm² of electrical device surface to which the agent is applied.

5370. The method of item 5169 wherein the agent is delivered from the electrical device, wherein a surface of the electrical device comprises about 1000 μg to about 2500 μg of the agent per mm² of electrical device surface to which the agent is applied.

5371. The method of item 5169, wherein the electrical device further comprises a coating, and the coating is a uniform coating.

5372. The method of item 5169, wherein the electrical device further comprises a coating, and the coating is a non-uniform coating.

5373. The method of item 5169, wherein the electrical device further comprises a coating, and the coating is a discontinuous coating.

5374. The method of item 5169, wherein the electrical device further comprises a coating, and the coating is a patterned coating.

5375. The method of item 5169, wherein the electrical device further comprises a coating, and the coating has a thickness of 100 μm or less.

5376. The method of item 5169, wherein the electrical device further comprises a coating, and the coating has a thickness of 10 μm or less.

5377. The method of item 5169, wherein the electrical device further comprises a coating, and the coating adheres to the surface of the electrical device upon deployment of the electrical device.

5378. The method of item 5169, wherein the electrical device further comprises a coating, and the coating is stable at room temperature for a period of at least 1 year.

5379. The method of item 5169, wherein the electrical device further comprises a coating, and the agent is present in the coating in an amount ranging between about 0.0001% to about 1% by weight.

5380. The method of item 5169, wherein the electrical device further comprises a coating, and the agent is present in the coating in an amount ranging between about 1% to about 10% by weight.

5381. The method of item 5169, wherein the electrical device further comprises a coating, and the agent is present in the coating in an amount ranging between about 10% to about 25% by weight.

5382. The method of item 5169, wherein the electrical device further comprises a coating, and the agent is present in the coating in an amount ranging between about 25% to about 70% by weight.

5383. The method of item 5169, wherein the electrical device further comprises a coating, and the coating comprises a polymer.

5384. The method of item 5169, wherein the electrical device comprises a first coating having a first composition and a second coating having a second composition.

5385. The method of item 5169, wherein the electrical device comprises a first coating having a first composition and a second coating having a second composition, wherein the first composition and the second composition are different.

5386. The method of item 5169 wherein the agent or the composition is affixed to the electrical device.

5387. The method of item 5169 wherein the agent or the composition is covalently attached to the electrical device.

5388. The method of item 5169 wherein the agent or the composition is non-covalently attached to the electrical device.

5389. The method of item 5169 wherein the electrical device comprises a coating that absorbs the agent or the composition.

5390. The method of item 5169 wherein the electrical device is interweaved with a thread composed of, or coated with, the agent or the composition.

5391. The method of item 5169 wherein a portion of the electrical device is covered with a sleeve that contains the agent or the composition.

5392. The method of item 5169 wherein the electrical device is completely covered with a sleeve that contains the agent or the composition.

5393. The method of item 5169 wherein a portion of the electrical device is covered with a mesh that contains the agent or the composition.

5394. The method of item 5169 wherein the electrical device is completely covered with a mesh that contains the agent or the composition.

5395. The method of item 5169 wherein the agent or the composition is applied to the electrical device surface prior to the placing of the electrical device into the host.

5396. The method of item 5169 wherein the agent or the composition is applied to the electrical device surface during the placing of the electrical device into the host.

5397. The method of item 5169 wherein the agent or the composition is applied to the electrical device surface immediately after the placing of the electrical device into the host.

5398. The method of item 5169 wherein the agent or the composition is applied to the surface of the tissue in the host surrounding the electrical device prior to the placing of the electrical device into the host.

5399. The method of item 5169 wherein the agent or the composition is applied to the surface of the tissue in the host surrounding the electrical device during the placing of the electrical device into the host.

5400. The method of item 5169 wherein the agent or the composition is applied to the surface of the tissue in the host surrounding the electrical device immediately after the placing of the electrical device into the host.

5401. The method of item 5169 wherein the agent or the composition is topically applied into the anatomical space where the electrical device is placed.

5402. The method of item 5169 wherein the agent or the composition is percutaneously injected into the tissue in the host surrounding the electrical device.

5403. The method for inhibiting scarring of any one of items 5169-5402 wherein the gastric nerve stimulator is adapted for treating or preventing morbid obesity.

5404. The method for inhibiting scarring of any one of items 5169-5402 wherein the gastric nerve stimulator is adapted for treating or preventing constipation.

5405. The method for inhibiting scarring of any one of items 5169-5402 wherein the gastric nerve stimulator comprises an electrical lead, an electrode and a stimulation generator.

5406. The method for inhibiting scarring of any one of items 5169-5402 wherein the gastric nerve stimulator comprises an electrical signal controller, connector wire and an attachment lead.

5407. A method for inhibiting scarring comprising placing a cochlear implant for treating deafness (i.e., an electrical device) and an anti-scarring agent or a composition comprising an ant-scarring agent into an animal host, wherein the agent inhibits scarring.

5408. The method of item 5407 wherein the agent is an adensosine A2A receptor antagonist.

5409. The method of item 5407 wherein the agent is an AKT inhibitor.

5410. The method of item 5407 wherein the agent is an alpha 2 integrin antagonist, wherein the alpha 2 integrin antagonist is Pharmaprojects No. 5754 (Merck KgaA).

5411. The method of item 5407 wherein the agent is an alpha 4 integrin antagonist.

5412. The method of item 5407 wherein the agent is an alpha 7 nicotinic receptor agonist.

5413. The method of item 5407 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

5414. The method of item 5407 wherein the agent is an apoptosis antagonist.

5415. The method of item 5407 wherein the agent is an apoptosis activator.

5416. The method of item 5407 wherein the agent is a beta 1 integrin antagonist.

5417. The method of item 5407 wherein the agent is a beta tubulin inhibitor.

5418. The method of item 5407 wherein the agent is a blocker of enzyme production in Hepatitis C.

5419. The method of item 5407 wherein the agent is a Bruton's tyrosine kinase inhibitor.

5420. The method of item 5407 wherein the agent is a calcineurin inhibitor.

5421. The method of item 5407 wherein the agent is a caspase 3 inhibitor.

5422. The method of item 5407 wherein the agent is a CC chemokine receptor antagonist.

5423. The method of item 5407 wherein the agent is a cell cycle inhibitor selected from the group consisting of SNS-595 (Sunesis), synthadotin, KRX-0403, and an analogue or derivative thereof.

5424. The method of item 5407 wherein the agent is a cathepsin B inhibitor.

5425. The method of item 5407 wherein the agent is a cathepsin K inhibitor, wherein the cathepsin K inhibitor is 462795 (GlaxoSmithKline), INPL-022-D6 (Amura Therapeutics), or an analogue or derivative thereof.

5426. The method of item 5407 wherein the agent is a cathepsin L inhibitor.

5427. The method of item 5407 wherein the agent is a CD40 antagonist.

5428. The method of item 5407 wherein the agent is a chemokine receptor agonist.

5429. The method of item 5407 wherein the agent is a chymase inhibitor.

5430. The method of item 5407 wherein the agent is a collagenase antagonist.

5431. The method of item 5407 wherein the agent is a CXCR antagonist.

5432. The method of item 5407 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

5433. The method of item 5407 wherein the agent is a cyclooxygenase 1 inhibitor.

5434. The method of item 5407 wherein the agent is a DHFR inhibitor.

5435. The method of item 5407 wherein the agent is a dual integrin inhibitor.

5436. The method of item 5407 wherein the agent is an elastase inhibitor.

5437. The method of item 5407 wherein the agent is an elongation factor-1 alpha inhibitor.

5438. The method of item 5407 wherein the agent is an endothelial growth factor antagonist.

5439. The method of item 5407 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

5440. The method of item 5407 wherein the agent is an endotoxin antagonist.

5441. The method of item 5407 wherein the agent is an epothilone and tubulin binder.

5442. The method of item 5407 wherein the agent is an estrogen receptor antagonist.

5443. The method of item 5407 wherein the agent is an FGF inhibitor.

5444. The method of item 5407 wherein the agent is a farnexyl transferase inhibitor.

5445. The method of item 5407 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

5446. The method of item 5407 wherein the agent is an FLT-3 kinase inhibitor.

5447. The method of item 5407 wherein the agent is an FGF receptor kinase inhibitor.

5448. The method of item 5407 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

5449. The method of item 5407 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

5450. The method of item 5407 wherein the agent is a histone deacetylase inhibitor.

5451. The method of item 5407 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

5452. The method of item 5407 wherein the agent is an ICAM inhibitor.

5453. The method of item 5407 wherein the agent is an IL, ICE and IRAK antagonist, wherein the antagonist is a CJ-14877, CP-424174 (Pfizer), NF-61 (Negma-Lerads), and an analogue or derivative thereof.

5454. The method of item 5407 wherein the agent is an IL-2 inhibitor.

5455. The method of item 5407 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

5456. The method of item 5407 wherein the agent is an IMPDH (inosine monophosphate).

5457. The method of item 5407 wherein the agent is an integrin antagonist.

5458. The method of item 5407 wherein the agent is an interleukin antagonist.

5459. The method of item 5407 wherein the agent is an inhibitor of type III receptor tyrosine kinase.

5460. The method of item 5407 wherein the agent is an irreversible inhibitor of enzyme methionine aminopeptidase type 2.

5461. The method of item 5407 wherein the agent is an isozyme selective delta protein kinase C inhibitor.

5462. The method of item 5407 wherein the agent a JAK3 enzyme inhibitor.

5463. The method of item 5407 wherein the agent is a JNK inhibitor.

5464. The method of item 5407 wherein the agent is a kinase inhibitor.

5465. The method of item 5407 wherein the agent is kinesin antagonist.

5466. The method of item 5407 wherein the agent is a kinesin antagonist.

5467. The method of item 5407 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

5468. The method of item 5407 wherein the agent is an MAP kinase inhibitor.

5469. The method of item 5407 wherein the agent is a matrix metalloproteinase inhibitor.

5470. The method of item 5407 wherein the agent is an MCP-CCR2 inhibitor.

5471. The method of item 5407 wherein the agent is an mTOR inhibitor.

5472. The method of item 5407 wherein the agent is an mTOR kinase inhibitor.

5473. The method of item 5407 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

5474. The method of item 5407 wherein the agent is an MIF inhibitor.

5475. The method of item 5407 wherein the agent is an MMP inhibitor.

5476. The method of item 5407 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (GAS No. 334476-64-1) (GlaxoSmithKline), Win-64821 (Sanofi-Aventis), PRX-96026 (Predix Pharmaceuticals), and an analogue or derivative thereof.

5477. The method of item 5407 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

5478. The method of item 5407 wherein the agent is a nitric oxide agonist.

5479. The method of item 5407 wherein the agent is an ornithine decarboxylase inhibitor.

5480. The method of item 5407 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

5481. The method of item 5407 wherein the agent is a palmitoyl-protein thioesterase inhibitor.

5482. The method of item 5407 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

5483. The method of item 5407 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

5484. The method of item 5407 wherein the agent is a phosphatase inhibitor.

5485. The method of item 5407 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-1 4-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

5486. The method of item 5407 wherein the agent is a PKC inhibitor.

5487. The method of item 5407 wherein the agent is a platelet activating factor antagonist.

5488. The method of item 5407 wherein the agent is a platelet-derived growth factor receptor kinase inhibitor.

5489. The method of item 5407 wherein the agent is a prolyl hydroxylase inhibitor.

5490. The method of item 5407 wherein the agent is a polymorphonuclear neutrophil inhibitor.

5491. The method of item 5407 wherein the agent is a protein kinase B inhibitor.

5492. The method of item 5407 wherein the agent is a protein kinase C stimulant.

5493. The method of item 5407 wherein the agent is a purine nucleoside analogue.

5494. The method of item 5407 wherein the agent is a purinoreceptor P2X antagonist.

5495. The method of item 5407 wherein the agent is a Raf kinase inhibitor.

5496. The method of item 5407 wherein the agent is a reversible inhibitor of ErbB1 and ErbB2.

5497. The method of item 5407 wherein the agent is a ribonucleoside triphosphate reductase inhibitor.

5498. The method of item 5407 wherein the agent is an SDF-1 antagonist.

5499. The method of item 5407 wherein the agent is a sheddase inhibitor.

5500. The method of item 5407 wherein the agent is an SRC inhibitor.

5501. The method of item 5407 wherein the agent is a stromelysin inhibitor.

5502. The method of item 5407 wherein the agent is an Syk kinase inhibitor.

5503. The method of item 5407 wherein the agent is a telomerase inhibitor.

5504. The method of item 5407 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

5505. The method of item 5407 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

5506. The method of item 5407 wherein the agent is a Toll receptor inhibitor.

5507. The method of item 5407 wherein the agent is a tubulin antagonist.

5508. The method of item 5407 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

5509. The method of item 5407 wherein the agent is a VEGF inhibitor.

5510. The method of item 5407 wherein the agent is a vitamin D receptor agonist.

5511. The method of item 5407 wherein the agent is ZD-6474 (an angiogenesis inhibitor).

5512. The method of item 5407 wherein the agent is AP-23573 (an mTOR inhibitor).

5513. The method of item 5407 wherein the agent is synthadotin (a tubulin antagonist).

5514. The method of item 5407 wherein the agent is S-0885 (a collagenase inhibitor).

5515. The method of item 5407 wherein the agent is aplidine (an elongation factor-1alpha inhibitor).

5516. The method of item 5407 wherein the agent is ixabepilone (an epithilone).

5517. The method of item 5407 wherein the agent is IDN-5390 (an angiogenesis inhibitor).

5518. The method of item 5407 wherein the agent is SB-2723005 (an angiogenesis inhibitor).

5519. The method of item 5407 wherein the agent is ABT-518 (an angiogenesis inhibitor).

5520. The method of item 5407 wherein the agent is combretastatin (an angiogenesis inhibitor).

5521. The method of item 5407 wherein the agent is anecortave acetate (an angiogenesis inhibitor).

5522. The method of item 5407 wherein the agent is SB-715992 (a kinesin antagonist).

5523. The method of item 5407 wherein the agent is temsirolimus (an mTOR inhibitor).

5524. The method of item 5407 wherein the agent is adalimumab (a TNFα antagonist).

5525. The method of item 5407, wherein the composition comprises a polymer.

5526. The method of item 5407, wherein the composition comprises a polymer, and the polymer is, or comprises, a copolymer.

5527. The method of item 5407, wherein the composition comprises a polymer, and the polymer is, or comprises, a block copolymer.

5528. The method of item 5407, wherein the composition comprises a polymer, and the polymer is, or comprises, a random copolymer.

5529. The method of item 5407, wherein the composition comprises a polymer, and the polymer is, or comprises, a biodegradable polymer.

5530. The method of item 5407, wherein the composition comprises a polymer, and the polymer is, or comprises, a non-biodegradable polymer.

5531. The method of item 5407, wherein the composition comprises a polymer, and the polymer is, or comprises, a hydrophilic polymer.

5532. The method of item 5407, wherein the composition comprises a polymer, and the polymer is, or comprises, a hydrophobic polymer.

5533. The method of item 5407, wherein the composition comprises a polymer, and the polymer is, or comprises, a polymer having hydrophilic domains.

5534. The method of item 5407, wherein the composition comprises a polymer, and the polymer is, or comprises, a polymer having hydrophobic domains.

5535. The method of item 5407, wherein the composition comprises a polymer, and the polymer is, or comprises, a non-conductive polymer.

5536. The method of item 5407, wherein the composition comprises a polymer, and the polymer is, or comprises, an elastomer.

5537. The method of item 5407, wherein the composition comprises a polymer, and the polymer is, or comprises, a hydrogel.

5538. The method of item 5407, wherein the composition comprises a polymer, and the polymer is, or comprises, a silicone polymer.

5539. The method of item 5407, wherein the composition comprises a polymer, and the polymer is, or comprises, a hydrocarbon polymer.

5540. The method of item 5407, wherein the composition comprises a polymer, and the polymer is, or comprises, a styrene-derived polymer.

5541. The method of item 5407, wherein the composition comprises a polymer, and the polymer is, or comprises, a butadiene-derived polymer.

5542. The method of item 5407, wherein the composition comprises a polymer, and the polymer is, or comprises, a macromer.

5543. The method of item 5407, wherein the composition comprises a polymer, and the polymer is, or comprises, a poly(ethylene glycol) polymer.

5544. The method of item 5407, wherein the composition comprises a polymer, and the polymer is, or comprises, an amorphous polymer.

5545. The method of item 5407, wherein the composition further comprises a second pharmaceutically active agent.

5546. The method of item 5407, wherein the composition further comprises an anti-inflammatory agent.

5547. The method of item 5407, wherein the composition further comprises an agent that inhibits infection.

5548. The method of item 5407, wherein the composition further comprises an anthracycline.

5549. The method of item 5407, wherein the composition further comprises doxorubicin.

5550. The method of item 5407 wherein the composition further comprises mitoxantrone.

5551. The method of item 5407 wherein the composition further comprises a fluoropyrimidine.

5552. The method of item 5407, wherein the composition further comprises 5-fluorouracil (5-FU).

5553. The method of item 5407, wherein the composition further comprises a folic acid antagonist.

5554. The method of item 5407, wherein the composition further comprises methotrexate.

5555. The method of item 5407, wherein the composition further comprises a podophylotoxin.

5556. The method of item 5407, wherein the composition further comprises etoposide.

5557. The method of item 5407, wherein the composition further comprises camptothecin.

5558. The method of item 5407, wherein the composition further comprises a hydroxyurea.

5559. The method of item 5407, wherein the composition further comprises a platinum complex.

5560. The method of item 5407, wherein the composition further comprises cisplatin.

5561. The method of item 5407 wherein the composition further comprises an anti-thrombotic agent.

5562. The method of item 5407, wherein the composition further comprises a visualization agent.

5563. The method of item 5407, wherein the composition further comprises a visualization agent, and the visualization agent is a radiopaque material, wherein the radiopaque material comprises a metal, a halogenated compound, or a barium containing compound.

5564. The method of item 5407, wherein the composition further comprises a visualization agent, and the visualization agent is, or comprises, barium, tantalum, or technetium.

5565. The method of item 5407, wherein the composition further comprises a visualization agent, and the visualization agent is, or comprises, an MRI responsive material.

5566. The method of item 5407, wherein the composition further comprises a visualization agent, and the visualization agent is, or comprises, a gadolinium chelate.

5567. The method of item 5407, wherein the composition further comprises a visualization agent, and the visualization agent is, or comprises, iron, magnesium, manganese, copper, or chromium.

5568. The method of item 5407, wherein the composition further comprises a visualization agent, and the visualization agent is, or comprises, iron oxide compound.

5569. The method of item 5407, wherein the composition further comprises a visualization agent, and the visualization agent is, or comprises, a dye, pigment, or colorant.

5570. The method of item 5407 wherein the agent is released in effective concentrations from the composition comprising the agent by diffusion over a period ranging from the time of administration to about 90 days.

5571. The method of item 5407 wherein the agent is released in effective concentrations from the composition comprising the agent by erosion of the composition over a period ranging from the time of administration to about 90 days.

5572. The method of item 5407 wherein the composition further comprises an inflammatory cytokine.

5573. The method of item 5407 wherein the composition further comprises an agent that stimulates cell proliferation.

5574. The method of item 5407 wherein the composition further comprises a polymeric carrier.

5575. The method of item 5407 wherein the composition is in the form of a gel, paste, or spray.

5576. The method of item 5407 wherein the electrical device is partially constructed with the agent or the composition.

5577. The method of item 5407 wherein the electrical device is impregnated with the agent or the composition.

5578. The method of item 5407, wherein the agent or the composition forms a coating, and the coating directly contacts the electrical device.

5579. The method of item 5407, wherein the agent or the composition forms a coating, and the coating indirectly contacts the electrical device.

5580. The method of item 5407 wherein the agent or the composition forms a coating, and the coating partially covers the electrical device.

5581. The method of item 5407, wherein the agent or the composition forms a coating, and the coating completely covers the electrical device.

5582. The method of item 5407 wherein the agent or the composition is located within pores or holes of the electrical device.

5583. The method of item 5407 wherein the agent or the composition is located within a channel, lumen, or divet of the electrical device.

5584. The method of item 5407 wherein the electrical device further comprises an echogenic material.

5585. The method of item 5407 wherein the electrical device further comprises an echogenic material, wherein the echogenic material is in the form of a coating.

5586. The method of item 5407 wherein the electrical device is sterile.

5587. The method of item 5407 wherein the agent is delivered from the electrical device, wherein the agent is released into tissue in the vicinity of the electrical device after deployment of the electrical device.

5588. The method of item 5407 wherein the agent is delivered from the electrical device, wherein the agent is released into tissue in the vicinity of the electrical device after deployment of the electrical device, wherein the tissue is connective tissue.

5589. The method of item 5407 wherein the agent is delivered from the electrical device, wherein the agent is released into tissue in the vicinity of the electrical device after deployment of the electrical device, wherein the tissue is muscle tissue.

5590. The method of item 5407 wherein the agent is delivered from the electrical device, wherein the agent is released into tissue in the vicinity of the electrical device after deployment of the electrical device, wherein the tissue is nerve tissue.

5591. The method of item 5407 wherein the agent is delivered from the electrical device, wherein the agent is released into tissue in the vicinity of the electrical device after deployment of the electrical device, wherein the tissue is epithelium tissue.

5592. The method of item 5407 wherein the agent is delivered from the electrical device, wherein the agent is released in effective concentrations from the electrical device over a period ranging from the time of deployment of the electrical device to about 1 year.

5593. The method of item 5407 wherein the agent is delivered from the electrical device, wherein the agent is released in effective concentrations from the electrical device over a period ranging from about 1 month to 6 months.

5594. The method of item 5407 wherein the agent is delivered from the electrical device, wherein the agent is released in effective concentrations from the electrical device over a period ranging from about 1-90 days.

5595. The method of item 5407 wherein the agent is delivered from the electrical device, wherein the agent is released in effective concentrations from the electrical device at a constant rate.

5596. The method of item 5407 wherein the agent is delivered from the electrical device, wherein the agent is released in effective concentrations from the electrical device at an increasing rate.

5597. The method of item 5407 wherein the agent is delivered from the electrical device, wherein the agent is released in effective concentrations from the electrical device at a decreasing rate.

5598. The method of item 5407 wherein the agent is delivered from the electrical device, wherein the electrical device comprises about 0.01 μg to about 10 μg of the agent.

5599. The method of item 5407 wherein the agent is delivered from the electrical device, wherein the electrical device comprises about 10 μg to about 10 mg of the agent.

5600. The method of item 5407 wherein the agent is delivered from the electrical device, wherein the electrical device comprises about 10 mg to about 250 mg of the agent.

5601. The method of item 5407 wherein the agent is delivered from the electrical device, wherein the electrical device comprises about 250 mg to about 1000 mg of the agent.

5602. The method of item 5407 wherein the agent is delivered from the electrical device, wherein the electrical device comprises about 1000 mg to about 2500 mg of the agent.

5603. The method of item 5407 wherein the agent is delivered from the electrical device, wherein a surface of the electrical device comprises less than 0.01 μg of the agent per mm² of electrical device surface to which the agent is applied.

5604. The method of item 5407 wherein the agent is delivered from the electrical device, wherein a surface of the electrical device comprises about 0.01 μg to about 1 μg of the agent per mm² of electrical device surface to which the agent is applied.

5605. The method of item 5407 wherein the agent is delivered from the electrical device, wherein a surface of the electrical device comprises about 1 μg to about 10 μg of the agent per mm² of electrical device surface to which the agent is applied.

5606. The method of item 5407 wherein the agent is delivered from the electrical device, wherein a surface of the electrical device comprises about 10 μg to about 250 μg of the agent per mm² of electrical device surface to which the agent is applied.

5607. The method of item 5407 wherein the agent is delivered from the electrical device, wherein a surface of the electrical device comprises about 250 μg to about 1000 μg of the agent per mm² of electrical device surface to which the agent is applied.

5608. The method of item 5407 wherein the agent is delivered from the electrical device, wherein a surface of the electrical device comprises about 1000 μg to about 2500 μg of the agent per mm² of electrical device surface to which the agent is applied.

5609. The method of item 5407, wherein the electrical device further comprises a coating, and the coating is a uniform coating.

5610. The method of item 5407, wherein the electrical device further comprises a coating, and the coating is a non-uniform coating.

5611. The method of item 5407, wherein the electrical device further comprises a coating, and the coating is a discontinuous coating.

5612. The method of item 5407, wherein the electrical device further comprises a coating, and the coating is a patterned coating.

5613. The method of item 5407, wherein the electrical device further comprises a coating, and the coating has a thickness of 100 μm or less.

5614. The method of item 5407, wherein the electrical device further comprises a coating, and the coating has a thickness of 10 μm or less.

5615. The method of item 5407, wherein the electrical device further comprises a coating, and the coating adheres to the surface of the electrical device upon deployment of the electrical device.

5616. The method of item 5407, wherein the electrical device further comprises a coating, and the coating is stable at room temperature for a period of at least 1 year.

5617. The method of item 5407, wherein the electrical device further comprises a coating, and the agent is present in the coating in an amount ranging between about 0.0001% to about 1% by weight.

5618. The method of item 5407, wherein the electrical device further comprises a coating, and the agent is present in the coating in an amount ranging between about 1% to about 10% by weight.

5619. The method of item 5407, wherein the electrical device further comprises a coating, and the agent is present in the coating in an amount ranging between about 10% to about 25% by weight.

5620. The method of item 5407, wherein the electrical device further comprises a coating, and the agent is present in the coating in an amount ranging between about 25% to about 70% by weight.

5621. The method of item 5407, wherein the electrical device further comprises a coating, and the coating comprises a polymer.

5622. The method of item 5407, wherein the electrical device comprises a first coating having a first composition and a second coating having a second composition.

5623. The method of item 5407, wherein the electrical device comprises a first coating having a first composition and a second coating having a second composition, wherein the first composition and the second composition are different.

5624. The method of item 5407 wherein the agent or the composition is affixed to the electrical device.

5625. The method of item 5407 wherein the agent or the composition is covalently attached to the electrical device.

5626. The method of item 5407 wherein the agent or the composition is non-covalently attached to the electrical device.

5627. The method of item 5407 wherein the electrical device comprises a coating that absorbs the agent or the composition.

5628. The method of item 5407 wherein the electrical device is interweaved with a thread composed of, or coated with, the agent or the composition.

5629. The method of item 5407 wherein a portion of the electrical device is covered with a sleeve that contains the agent or the composition.

5630. The method of item 5407 wherein the electrical device is completely covered with a sleeve that contains the agent or the composition.

5631. The method of item 5407 wherein a portion of the electrical device is covered with a mesh that contains the agent or the composition.

5632. The method of item 5407 wherein the electrical device is completely covered with a mesh that contains the agent or the composition.

5633. The method of item 5407 wherein the agent or the composition is applied to the electrical device surface prior to the placing of the electrical device into the host.

5634. The method of item 5407 wherein the agent or the composition is applied to the electrical device surface during the placing of the electrical device into the host.

5635. The method of item 5407 wherein the agent or the composition is applied to the electrical device surface immediately after the placing of the electrical device into the host.

5636. The method of item 5407 wherein the agent or the composition is applied to the surface of the tissue in the host surrounding the electrical device prior to the placing of the electrical device into the host.

5637. The method of item 5407 wherein the agent or the composition is applied to the surface of the tissue in the host surrounding the electrical device during the placing of the electrical device into the host.

5638. The method of item 5407 wherein the agent or the composition is applied to the surface of the tissue in the host surrounding the electrical device immediately after the placing of the electrical device into the host.

5639. The method of item 5407 wherein the agent or the composition is topically applied into the anatomical space where the electrical device is placed.

5640. The method of item 5407 wherein the agent or the composition is percutaneously injected into the tissue in the host surrounding the electrical device.

5641. The method for inhibiting scarring of any one of items 5407-5640 wherein the cochlear implant comprises a plurality of transducer elements.

5642. The method for inhibiting scarring of any one of items 5407-5640 wherein the cochlear implant comprises a sound-to-electrical stimulation encoder, a body implantable receiver-stimulator, and electrodes.

5643. The method for inhibiting scarring of any one of items 5407-5640 wherein the cochlear implant comprises a transducer and an electrode array.

5644. The method for inhibiting scarring of any one of items 5407-5640 wherein the cochlear implant is a subcranially implantable electromechanical system.

5645. A method for inhibiting scarring comprising placing a bone growth stimulator (i.e., an electrical device) and an anti-scarring agent or a composition comprising an ant-scarring agent into an animal host, wherein the agent inhibits scarring.

5646. The method of item 5645 wherein the agent is an adensosine A2A receptor antagonist.

5647. The method of item 5645 wherein the agent is an AKT inhibitor.

5648. The method of item 5645 wherein the agent is an alpha 2 integrin antagonist, wherein the alpha 2 integrin antagonist is Pharmaprojects No. 5754 (Merck KgaA).

5649. The method of item 5645 wherein the agent is an alpha 4 integrin antagonist.

5650. The method of item 5645 wherein the agent is an alpha 7 nicotinic receptor agonist.

5651. The method of item 5645 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

5652. The method of item 5645 wherein the agent is an apoptosis antagonist.

5653. The method of item 5645 wherein the agent is an apoptosis activator.

5654. The method of item 5645 wherein the agent is a beta 1 integrin antagonist.

5655. The method of item 5645 wherein the agent is a beta tubulin inhibitor.

5656. The method of item 5645 wherein the agent is a blocker of enzyme production in Hepatitis C.

5657. The method of item 5645 wherein the agent is a Bruton's tyrosine kinase inhibitor.

5658. The method of item 5645 wherein the agent is a calcineurin inhibitor.

5659. The method of item 5645 wherein the agent is a caspase 3 inhibitor.

5660. The method of item 5645 wherein the agent is a CC chemokine receptor antagonist.

5661. The method of item 5645 wherein the agent is a cell cycle inhibitor selected from the group consisting of SNS-595 (Sunesis), synthadotin, KRX-0403, and an analogue or derivative thereof.

5662. The method of item 5645 wherein the agent is a cathepsin B inhibitor.

5663. The method of item 5645 wherein the agent is a cathepsin K inhibitor, wherein the cathepsin K inhibitor is 462795 (GlaxoSmithKline), INPL-022-D6 (Amura Therapeutics), or an analogue or derivative thereof.

5664. The method of item 5645 wherein the agent is a cathepsin L inhibitor.

5665. The method of item 5645 wherein the agent is a CD40 antagonist.

5666. The method of item 5645 wherein the agent is a chemokine receptor agonist.

5667. The method of item 5645 wherein the agent is a chymase inhibitor.

5668. The method of item 5645 wherein the agent is a collagenase antagonist.

5669. The method of item 5645 wherein the agent is a CXCR antagonist.

5670. The method of item 5645 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

5671. The method of item 5645 wherein the agent is a cyclooxygenase 1 inhibitor.

5672. The method of item 5645 wherein the agent is a DHFR inhibitor.

5673. The method of item 5645 wherein the agent is a dual integrin inhibitor.

5674. The method of item 5645 wherein the agent is an elastase inhibitor.

5675. The method of item 5645 wherein the agent is an elongation factor-1 alpha inhibitor.

5676. The method of item 5645 wherein the agent is an endothelial growth factor antagonist.

5677. The method of item 5645 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

5678. The method of item 5645 wherein the agent is an endotoxin antagonist.

5679. The method of item 5645 wherein the agent is an epothilone and tubulin binder.

5680. The method of item 5645 wherein the agent is an estrogen receptor antagonist.

5681. The method of item 5645 wherein the agent is an FGF inhibitor.

5682. The method of item 5645 wherein the agent is a farnexyl transferase inhibitor.

5683. The method of item 5645 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

5684. The method of item 5645 wherein the agent is an FLT-3 kinase inhibitor.

5685. The method of item 5645 wherein the agent is an FGF receptor kinase inhibitor.

5686. The method of item 5645 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

5687. The method of item 5645 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

5688. The method of item 5645 wherein the agent is a histone deacetylase inhibitor.

5689. The method of item 5645 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof

5690. The method of item 5645 wherein the agent is an ICAM inhibitor.

5691. The method of item 5645 wherein the agent is an IL, ICE and IRAK antagonist, wherein the antagonist is a CJ-14877, CP-424174 (Pfizer), NF-61 (Negma-Lerads), and an analogue or derivative thereof.

5692. The method of item 5645 wherein the agent is an IL-2 inhibitor.

5693. The method of item 5645 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

5694. The method of item 5645 wherein the agent is an IMPDH (inosine monophosphate).

5695. The method of item 5645 wherein the agent is an integrin antagonist.

5696. The method of item 5645 wherein the agent is an interleukin antagonist.

5697. The method of item 5645 wherein the agent is an inhibitor of type III receptor tyrosine kinase.

5698. The method of item 5645 wherein the agent is an irreversible inhibitor of enzyme methionine aminopeptidase type 2.

5699. The method of item 5645 wherein the agent is an isozyme selective delta protein kinase C inhibitor.

5700. The method of item 5645 wherein the agent a JAK3 enzyme inhibitor.

5701. The method of item 5645 wherein the agent is a JNK inhibitor.

5702. The method of item 5645 wherein the agent is a kinase inhibitor.

5703. The method of item 5645 wherein the agent is kinesin antagonist.

5704. The method of item 5645 wherein the agent is a kinesin antagonist.

5705. The method of item 5645 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

5706. The method of item 5645 wherein the agent is an MAP kinase inhibitor.

5707. The method of item 5645 wherein the agent is a matrix metalloproteinase inhibitor.

5708. The method of item 5645 wherein the agent is an MCP-CCR2 inhibitor.

5709. The method of item 5645 wherein the agent is an mTOR inhibitor.

5710. The method of item 5645 wherein the agent is an mTOR kinase inhibitor.

5711. The method of item 5645 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DMI (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

5712. The method of item 5645 wherein the agent is an MIF inhibitor.

5713. The method of item 5645 wherein the agent is an MMP inhibitor.

5714. The method of item 5645 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

5715. The method of item 5645 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-494) (Encore Pharmaceuticals), and an analogue or derivative thereof.

5716. The method of item 5645 wherein the agent is a nitric oxide agonist.

5717. The method of item 5645 wherein the agent is an ornithine decarboxylase inhibitor.

5718. The method of item 5645 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

5719. The method of item 5645 wherein the agent is a palmitoyl-protein thioesterase inhibitor.

5720. The method of item 5645 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

5721. The method of item 5645 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-734 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

5722. The method of item 5645 wherein the agent is a phosphatase inhibitor.

5723. The method of item 5645 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

5724. The method of item 5645 wherein the agent is a PKC inhibitor.

5725. The method of item 5645 wherein the agent is a platelet activating factor antagonist.

5726. The method of item 5645 wherein the agent is a platelet-derived growth factor receptor kinase inhibitor.

5727. The method of item 5645 wherein the agent is a prolyl hydroxylase inhibitor.

5728. The method of item 5645 wherein the agent is a polymorphonuclear neutrophil inhibitor.

5729. The method of item 5645 wherein the agent is a protein kinase B inhibitor.

5730. The method of item 5645 wherein the agent is a protein kinase C stimulant.

5731. The method of item 5645 wherein the agent is a purine nucleoside analogue.

5732. The method of item 5645 wherein the agent is a purinoreceptor P2X antagonist.

5733. The method of item 5645 wherein the agent is a Raf kinase inhibitor.

5734. The method of item 5645 wherein the agent is a reversible inhibitor of ErbB1 and ErbB2.

5735. The method of item 5645 wherein the agent is a ribonucleoside triphosphate reductase inhibitor.

5736. The method of item 5645 wherein the agent is an SDF-1 antagonist.

5737. The method of item 5645 wherein the agent is a sheddase inhibitor.

5738. The method of item 5645 wherein the agent is an SRC inhibitor.

5739. The method of item 5645 wherein the agent is a stromelysin inhibitor.

5740. The method of item 5645 wherein the agent is an Syk kinase inhibitor.

5741. The method of item 5645 wherein the agent is a telomerase inhibitor.

5742. The method of item 5645 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

5743. The method of item 5645 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

5744. The method of item 5645 wherein the agent is a Toll receptor inhibitor.

5745. The method of item 5645 wherein the agent is a tubulin antagonist.

5746. The method of item 5645 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

5747. The method of item 5645 wherein the agent is a VEGF inhibitor.

5748. The method of item 5645 wherein the agent is a vitamin D receptor agonist.

5749. The method of item 5645 wherein the agent is ZD-6474 (an angiogenesis inhibitor).

5750. The method of item 5645 wherein the agent is AP-23573 (an mTOR inhibitor).

5751. The method of item 5645 wherein the agent is synthadotin (a tubulin antagonist).

5752. The method of item 5645 wherein the agent is S-0885 (a collagenase inhibitor).

5753. The method of item 5645 wherein the agent is aplidine (an elongation factor-1 alpha inhibitor).

5754. The method of item 5645 wherein the agent is ixabepilone (an epithilone).

5755. The method of item 5645 wherein the agent is IDN-5390 (an angiogenesis inhibitor).

5756. The method of item 5645 wherein the agent is SB-2723005 (an angiogenesis inhibitor).

5757. The method of item 5645 wherein the agent is ABT-518 (an angiogenesis inhibitor).

5758. The method of item 5645 wherein the agent is combretastatin (an angiogenesis inhibitor).

5759. The method of item 5645 wherein the agent is anecortave acetate (an angiogenesis inhibitor).

5760. The method of item 5645 wherein the agent is SB-715992 (a kinesin antagonist).

5761. The method of item 5645 wherein the agent is temsirolimus (an mTOR inhibitor).

5762. The method of item 5645 wherein the agent is adalimumab (a TNFα antagonist).

5763. The method of item 5645, wherein the composition comprises a polymer.

5764. The method of item 5645, wherein the composition comprises a polymer, and the polymer is, or comprises, a copolymer.

5765. The method of item 5645, wherein the composition comprises a polymer, and the polymer is, or comprises, a block copolymer.

5766. The method of item 5645, wherein the composition comprises a polymer, and the polymer is, or comprises, a random copolymer.

5767. The method of item 5645, wherein the composition comprises a polymer, and the polymer is, or comprises, a biodegradable polymer.

5768. The method of item 5645, wherein the composition comprises a polymer, and the polymer is, or comprises, a non-biodegradable polymer.

5769. The method of item 5645, wherein the composition comprises a polymer, and the polymer is, or comprises, a hydrophilic polymer.

5770. The method of item 5645, wherein the composition comprises a polymer, and the polymer is, or comprises, a hydrophobic polymer.

5771. The method of item 5645, wherein the composition comprises a polymer, and the polymer is, or comprises, a polymer having hydrophilic domains.

5772. The method of item 5645, wherein the composition comprises a polymer, and the polymer is, or comprises, a polymer having hydrophobic domains.

5773. The method of item 5645, wherein the composition comprises a polymer, and the polymer is, or comprises, a non-conductive polymer.

5774. The method of item 5645, wherein the composition comprises a polymer, and the polymer is, or comprises, an elastomer.

5775. The method of item 5645, wherein the composition comprises a polymer, and the polymer is, or comprises, a hydrogel

5776. The method of item 5645, wherein the composition comprises a polymer, and the polymer is, or comprises, a silicone polymer.

5777. The method of item 5645, wherein the composition comprises a polymer, and the polymer is, or comprises, a hydrocarbon polymer.

5778. The method of item 5645, wherein the composition comprises a polymer, and the polymer is, or comprises, a styrene-derived polymer

5779. The method of item 5645, wherein the composition comprises a polymer, and the polymer is, or comprises, a butadiene-derived polymer.

5780. The method of item 5645, wherein the composition comprises a polymer, and the polymer is, or comprises, a macromer.

5781. The method of item 5645, wherein the composition comprises a polymer, and the polymer is, or comprises, a poly(ethylene glycol) polymer.

5782. The method of item 5645, wherein the composition comprises a polymer, and the polymer is, or comprises, an amorphous polymer.

5783. The method of item 5645, wherein the composition further comprises a second pharmaceutically active agent.

5784. The method of item 5645, wherein the composition further comprises an anti-inflammatory agent.

5785. The method of item 5645, wherein the composition further comprises an agent that inhibits infection.

5786. The method of item 5645, wherein the composition further comprises an anthracycline.

5787. The method of item 5645, wherein the composition further comprises doxorubicin.

5788. The method of item 5645 wherein the composition further comprises mitoxantrone.

5789. The method of item 5645 wherein the composition further comprises a fluoropyrimidine.

5790. The method of item 5645, wherein the composition further comprises 5-fluorouracil (5-FU).

5791. The method of item 5645, wherein the composition further comprises a folic acid antagonist.

5792. The method of item 5645, wherein the composition further comprises methotrexate.

5793. The method of item 5645, wherein the composition further comprises a podophylotoxin.

5794. The method of item 5645, wherein the composition further comprises etoposide.

5795. The method of item 5645, wherein the composition further comprises camptothecin.

5796. The method of item 5645, wherein the composition further comprises a hydroxyurea.

5797. The method of item 5645, wherein the composition further comprises a platinum complex.

5798. The method of item 5645, wherein the composition further comprises cisplatin.

5799. The method of item 5645 wherein the composition further comprises an anti-thrombotic agent.

5800. The method of item 5645, wherein the composition further comprises a visualization agent.

5801. The method of item 5645, wherein the composition further comprises a visualization agent, and the visualization agent is a radiopaque material, wherein the radiopaque material comprises a metal, a halogenated compound, or a barium containing compound.

5802. The method of item 5645, wherein the composition further comprises a visualization agent, and the visualization agent is, or comprises, barium, tantalum, or technetium.

5803. The method of item 5645, wherein the composition further comprises a visualization agent, and the visualization agent is, or comprises, an MRI responsive material.

5804. The method of item 5645, wherein the composition further comprises a visualization agent, and the visualization agent is, or comprises, a gadolinium chelate.

5805. The method of item 5645, wherein the composition further comprises a visualization agent, and the visualization agent is, or comprises, iron, magnesium, manganese, copper, or chromium.

5806. The method of item 5645, wherein the composition further comprises a visualization agent, and the visualization agent is, or comprises, iron oxide compound.

5807. The method of item 5645, wherein the composition further comprises a visualization agent, and the visualization agent is, or comprises, a dye, pigment, or colorant.

5808. The method of item 5645 wherein the agent is released in effective concentrations from the composition comprising the agent by diffusion over a period ranging from the time of administration to about 90 days.

5809. The method of item 5645 wherein the agent is released in effective concentrations from the composition comprising the agent by erosion of the composition over a period ranging from the time of administration to about 90 days.

5810. The method of item 5645 wherein the composition further comprises an inflammatory cytokine.

5811. The method of item 5645 wherein the composition further comprises an agent that stimulates cell proliferation.

5812. The method of item 5645 wherein the composition further comprises a polymeric carrier.

5813. The method of item 5645 wherein the composition is in the form of a gel, paste, or spray.

5814. The method of item 5645 wherein the electrical device is partially constructed with the agent or the composition.

5815. The method of item 5645 wherein the electrical device is impregnated with the agent or the composition.

5816. The method of item 5645, wherein the agent or the composition forms a coating, and the coating directly contacts the electrical device.

5817. The method of item 5645, wherein the agent or the composition forms a coating, and the coating indirectly contacts the electrical device.

5818. The method of item 5645 wherein the agent or the composition forms a coating, and the coating partially covers the electrical device.

5819. The method of item 5645, wherein the agent or the composition forms a coating, and the coating completely covers the electrical device.

5820. The method of item 5645 wherein the agent or the composition is located within pores or holes of the electrical device.

5821. The method of item 5645 wherein the agent or the composition is located within a channel, lumen, or divet of the electrical device.

5822. The method of item 5645 wherein the electrical device further comprises an echogenic material.

5823. The method of item 5645 wherein the electrical device further comprises an echogenic material, wherein the echogenic material is in the form of a coating.

5824. The method of item 5645 wherein the electrical device is sterile.

5825. The method of item 5645 wherein the agent is delivered from the electrical device, wherein the agent is released into tissue in the vicinity of the electrical device after deployment of the electrical device.

5826. The method of item 5645 wherein the agent is delivered from the electrical device, wherein the agent is released into tissue in the vicinity of the electrical device after deployment of the electrical device, wherein the tissue is connective tissue.

5827. The method of item 5645 wherein the agent is delivered from the electrical device, wherein the agent is released into tissue in the vicinity of the electrical device after deployment of the electrical device, wherein the tissue is muscle tissue.

5828. The method of item 5645 wherein the agent is delivered from the electrical device, wherein the agent is released into tissue in the vicinity of the electrical device after deployment of the electrical device, wherein the tissue is nerve tissue.

5829. The method of item 5645 wherein the agent is delivered from the electrical device, wherein the agent is released into tissue in the vicinity of the electrical device after deployment of the electrical device, wherein the tissue is epithelium tissue.

5830. The method of item 5645 wherein the agent is delivered from the electrical device, wherein the agent is released in effective concentrations from the electrical device over a period ranging from the time of deployment of the electrical device to about 1 year.

5831. The method of item 5645 wherein the agent is delivered from the electrical device, wherein the agent is released in effective concentrations from the electrical device over a period ranging from about 1 month to 6 months.

5832. The method of item 5645 wherein the agent is delivered from the electrical device, wherein the agent is released in effective concentrations from the electrical device over a period ranging from about 1-90 days.

5833. The method of item 5645 wherein the agent is delivered from the electrical device, wherein the agent is released in effective concentrations from the electrical device at a constant rate.

5834. The method of item 5645 wherein the agent is delivered from the electrical device, wherein the agent is released in effective concentrations from the electrical device at an increasing rate.

5835. The method of item 5645 wherein the agent is delivered from the electrical device, wherein the agent is released in effective concentrations from the electrical device at a decreasing rate.

5836. The method of item 5645 wherein the agent is delivered from the electrical device, wherein the electrical device comprises about 0.01 μg to about 10 μg of the agent.

5837. The method of item 5645 wherein the agent is delivered from the electrical device, wherein the electrical device comprises about 10 μg to about 10 mg of the agent.

5838. The method of item 5645 wherein the agent is delivered from the electrical device, wherein the electrical device comprises about 10 mg to about 250 mg of the agent.

5839. The method of item 5645 wherein the agent is delivered from the electrical device, wherein the electrical device comprises about 250 mg to about 1000 mg of the agent.

5840. The method of item 5645 wherein the agent is delivered from the electrical device, wherein the electrical device comprises about 1000 mg to about 2500 mg of the agent.

5841. The method of item 5645 wherein the agent is delivered from the electrical device, wherein a surface of the electrical device comprises less than 0.01 μg of the agent per mm² of electrical device surface to which the agent is applied.

5842. The method of item 5645 wherein the agent is delivered from the electrical device, wherein a surface of the electrical device comprises about 0.01 μg to about 1 μg of the agent per mm² of electrical device surface to which the agent is applied.

5843. The method of item 5645 wherein the agent is delivered from the electrical device, wherein a surface of the electrical device comprises about 1 μg to about 10 μg of the agent per mm² of electrical device surface to which the agent is applied.

5844. The method of item 5645 wherein the agent is delivered from the electrical device, wherein a surface of the electrical device comprises about 10 μg to about 250 μg of the agent per mm² of electrical device surface to which the agent is applied.

5845. The method of item 5645 wherein the agent is delivered from the electrical device, wherein a surface of the electrical device comprises about 250 μg to about 1000 μg of the agent per mm² of electrical device surface to which the agent is applied.

5846. The method of item 5645 wherein the agent is delivered from the electrical device, wherein a surface of the electrical device comprises about 1000 μg to about 2500 μg of the agent per mm² of electrical device surface to which the agent is applied.

5847. The method of item 5645, wherein the electrical device further comprises a coating, and the coating is a uniform coating.

5848. The method of item 5645, wherein the electrical device further comprises a coating, and the coating is a non-uniform coating.

5849. The method of item 5645, wherein the electrical device further comprises a coating, and the coating is a discontinuous coating.

5850. The method of item 5645, wherein the electrical device further comprises a coating, and the coating is a patterned coating.

5851. The method of item 5645, wherein the electrical device further comprises a coating, and the coating has a thickness of 100 μm or less.

5852. The method of item 5645, wherein the electrical device further comprises a coating, and the coating has a thickness of 10 μm or less.

5853. The method of item 5645, wherein the electrical device further comprises a coating, and the coating adheres to the surface of the electrical device upon deployment of the electrical device.

5854. The method of item 5645, wherein the electrical device further comprises a coating, and the coating is stable at room temperature for a period of at least 1 year.

5855. The method of item 5645, wherein the electrical device further comprises a coating, and the agent is present in the coating in an amount ranging between about 0.0001% to about 1% by weight.

5856. The method of item 5645, wherein the electrical device further comprises a coating, and the agent is present in the coating in an amount ranging between about 1% to about 10% by weight.

5857. The method of item 5645, wherein the electrical device further comprises a coating, and the agent is present in the coating in an amount ranging between about 10% to about 25% by weight.

5858. The method of item 5645, wherein the electrical device further comprises a coating, and the agent is present in the coating in an amount ranging between about 25% to about 70% by weight.

5859. The method of item 5645, wherein the electrical device further comprises a coating, and the coating comprises a polymer.

5860. The method of item 5645, wherein the electrical device comprises a first coating having a first composition and a second coating having a second composition.

5861. The method of item 5645, wherein the electrical device comprises a first coating having a first composition and a second coating having a second composition, wherein the first composition and the second composition are different.

5862. The method of item 5645 wherein the agent or the composition is affixed to the electrical device.

5863. The method of item 5645 wherein the agent or the composition is covalently attached to the electrical device.

5864. The method of item 5645 wherein the agent or the composition is non-covalently attached to the electrical device.

5865. The method of item 5645 wherein the electrical device comprises a coating that absorbs the agent or the composition.

5866. The method of item 5645 wherein the electrical device is interweaved with a thread composed of, or coated with, the agent or the composition.

5867. The method of item 5645 wherein a portion of the electrical device is covered with a sleeve that contains the agent or the composition.

5868. The method of item 5645 wherein the electrical device is completely covered with a sleeve that contains the agent or the composition.

5869. The method of item 5645 wherein a portion of the electrical device is covered with a mesh that contains the agent or the composition.

5870. The method of item 5645 wherein the electrical device is completely covered with a mesh that contains the agent or the composition.

5871. The method of item 5645 wherein the agent or the composition is applied to the electrical device surface prior to the placing of the electrical device into the host.

5872. The method of item 5645 wherein the agent or the composition is applied to the electrical device surface during the placing of the electrical device into the host.

5873. The method of item 5645 wherein the agent or the composition is applied to the electrical device surface immediately after the placing of the electrical device into the host.

5874. The method of item 5645 wherein the agent or the composition is applied to the surface of the tissue in the host surrounding the electrical device prior to the placing of the electrical device into the host.

5875. The method of item 5645 wherein the agent or the composition is applied to the surface of the tissue in the host surrounding the electrical device during the placing of the electrical device into the host.

5876. The method of item 5645 wherein the agent or the composition is applied to the surface of the tissue in the host surrounding the electrical device immediately after the placing of the electrical device into the host.

5877. The method of item 5645 wherein the agent or the composition is topically applied into the anatomical space where the electrical device is placed.

5878. The method of item 5645 wherein the agent or the composition is percutaneously injected into the tissue in the host surrounding the electrical device.

5879. The method for inhibiting scarring of any one of items 5645-5878 wherein the bone growth stimulator comprises an electrode and a generator having a strain response piezoelectric material that responds to strain.

5880. A method for inhibiting scarring comprising placing a cardiac pacemaker (i.e., an electrical device) and an anti-scarring agent or a composition comprising an ant-scarring agent into an animal host, wherein the agent inhibits scarring.

5881. The method of item 5880 wherein the agent is an adensosine A2A receptor antagonist.

5882. The method of item 5880 wherein the agent is an AKT inhibitor.

5883. The method of item 5880 wherein the agent is an alpha 2 integrin antagonist, wherein the alpha 2 integrin antagonist is Pharmaprojects No. 5754 (Merck KgaA).

5884. The method of item 5880 wherein the agent is an alpha 4 integrin antagonist.

5885. The method of item 5880 wherein the agent is an alpha 7 nicotinic receptor agonist.

5886. The method of item 5880 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

5887. The method of item 5880 wherein the agent is an apoptosis antagonist.

5888. The method of item 5880 wherein the agent is an apoptosis activator.

5889. The method of item 5880 wherein the agent is a beta 1 integrin antagonist.

5890. The method of item 5880 wherein the agent is a beta tubulin inhibitor.

5891. The method of item 5880 wherein the agent is a blocker of enzyme production in Hepatitis C.

5892. The method of item 5880 wherein the agent is a Bruton's tyrosine kinase inhibitor.

5893. The method of item 5880 wherein the agent is a calcineurin inhibitor.

5894. The method of item 5880 wherein the agent is a caspase 3 inhibitor.

5895. The method of item 5880 wherein the agent is a CC chemokine receptor antagonist.

5896. The method of item 5880 wherein the agent is a cell cycle inhibitor selected from the group consisting of SNS-595 (Sunesis), synthadotin, KRX-0403, and an analogue or derivative thereof.

5897. The method of item 5880 wherein the agent is a cathepsin B inhibitor.

5898. The method of item 5880 wherein the agent is a cathepsin K inhibitor, wherein the cathepsin K inhibitor is 462795 (GlaxoSmithKline), INPL-022-D6 (Amura Therapeutics), or an analogue or derivative thereof.

5899. The method of item 5880 wherein the agent is a cathepsin L inhibitor.

5900. The method of item 5880 wherein the agent is a CD40 antagonist.

5901. The method of item 5880 wherein the agent is a chemokine receptor agonist.

5902. The method of item 5880 wherein the agent is a chymase inhibitor.

5903. The method of item 5880 wherein the agent is a collagenase antagonist.

5904. The method of item 5880 wherein the agent is a CXCR antagonist.

5905. The method of item 5880 wherein the agent is a cyclin dependent kinase inhibitor selected from the group consisting of a CDK-1 inhibitor, a CDK-2 inhibitor, a CDK-4 inhibitor, a CDK-6 inhibitor, a CAKI inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

5906. The method of item 5880 wherein the agent is a cyclooxygenase 1 inhibitor.

5907. The method of item 5880 wherein the agent is a DHFR inhibitor.

5908. The method of item 5880 wherein the agent is a dual integrin inhibitor.

5909. The method of item 5880 wherein the agent is an elastase inhibitor.

5910. The method of item 5880 wherein the agent is an elongation factor-1 alpha inhibitor.

5911. The method of item 5880 wherein the agent is an endothelial growth factor antagonist.

5912. The method of item 5880 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

5913. The method of item 5880 wherein the agent is an endotoxin antagonist.

5914. The method of item 5880 wherein the agent is an epothilone and tubulin binder.

5915. The method of item 5880 wherein the agent is an estrogen receptor antagonist.

5916. The method of item 5880 wherein the agent is an FGF inhibitor.

5917. The method of item 5880 wherein the agent is a farnexyl transferase inhibitor.

5918. The method of item 5880 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

5919. The method of item 5880 wherein the agent is an FLT-3 kinase inhibitor.

5920. The method of item 5880 wherein the agent is an FGF receptor kinase inhibitor.

5921. The method of item 5880 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

5922. The method of item 5880 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

5923. The method of item 5880 wherein the agent is a histone deacetylase inhibitor.

5924. The method of item 5880 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

5925. The method of item 5880 wherein the agent is an ICAM inhibitor.

5926. The method of item 5880 wherein the agent is an IL, ICE and IRAK antagonist, wherein the antagonist is a CJ-14877, CP-424174 (Pfizer), NF-61 (Negma-Lerads), and an analogue or derivative thereof.

5927. The method of item 5880 wherein the agent is an IL-2 inhibitor.

5928. The method of item 5880 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

5929. The method of item 5880 wherein the agent is an IMPDH (inosine monophosphate).

5930. The method of item 5880 wherein the agent is an integrin antagonist.

5931. The method of item 5880 wherein the agent is an interleukin antagonist.

5932. The method of item 5880 wherein the agent is an inhibitor of type III receptor tyrosine kinase.

5933. The method of item 5880 wherein the agent is an irreversible inhibitor of enzyme methionine aminopeptidase type 2.

5934. The method of item 5880 wherein the agent is an isozyme selective delta protein kinase C inhibitor.

5935. The method of item 5880 wherein the agent a JAK3 enzyme inhibitor.

5936. The method of item 5880 wherein the agent is a JNK inhibitor.

5937. The method of item 5880 wherein the agent is a kinase inhibitor.

5938. The method of item 5880 wherein the agent is kinesin antagonist.

5939. The method of item 5880 wherein the agent is a kinesin antagonist.

5940. The method of item 5880 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

5941. The method of item 5880 wherein the agent is an MAP kinase inhibitor.

5942. The method of item 5880 wherein the agent is a matrix metalloproteinase inhibitor.

5943. The method of item 5880 wherein the agent is an MCP-CCR2 inhibitor.

5944. The method of item 5880 wherein the agent is an mTOR inhibitor.

5945. The method of item 5880 wherein the agent is an mTOR kinase inhibitor.

5946. The method of item 5880 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

5947. The method of item 5880 wherein the agent is an MIF inhibitor.

5948. The method of item 5880 wherein the agent is an MMP inhibitor.

5949. The method of item 5880 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

5950. The method of item 5880 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

5951. The method of item 5880 wherein the agent is a nitric oxide agonist.

5952. The method of item 5880 wherein the agent is an ornithine decarboxylase inhibitor.

5953. The method of item 5880 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

5954. The method of item 5880 wherein the agent is a palmitoyl-protein thioesterase inhibitor.

5955. The method of item 5880 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

5956. The method of item 5880 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

5957. The method of item 5880 wherein the agent is a phosphatase inhibitor.

5958. The method of item 5880 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

5959. The method of item 5880 wherein the agent is a PKC inhibitor.

5960. The method of item 5880 wherein the agent is a platelet activating factor antagonist.

5961. The method of item 5880 wherein the agent is a platelet-derived growth factor receptor kinase inhibitor.

5962. The method of item 5880 wherein the agent is a prolyl hydroxylase inhibitor.

5963. The method of item 5880 wherein the agent is a polymorphonuclear neutrophil inhibitor.

5964. The method of item 5880 wherein the agent is a protein kinase B inhibitor.

5965. The method of item 5880 wherein the agent is a protein kinase C stimulant.

5966. The method of item 5880 wherein the agent is a purine nucleoside analogue.

5967. The method of item 5880 wherein the agent is a purinoreceptor P2X antagonist.

5968. The method of item 5880 wherein the agent is a Raf kinase inhibitor.

5969. The method of item 5880 wherein the agent is a reversible inhibitor of ErbB1 and ErbB2.

5970. The method of item 5880 wherein the agent is a ribonucleoside triphosphate reductase inhibitor.

5971. The method of item 5880 wherein the agent is an SDF-1 antagonist.

5972. The method of item 5880 wherein the agent is a sheddase inhibitor.

5973. The method of item 5880 wherein the agent is an SRC inhibitor.

5974. The method of item 5880 wherein the agent is a stromelysin inhibitor.

5975. The method of item 5880 wherein the agent is an Syk kinase inhibitor.

5976. The method of item 5880 wherein the agent is a telomerase inhibitor.

5977. The method of item 5880 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

5978. The method of item 5880 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), vT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

5979. The method of item 5880 wherein the agent is a Toll receptor inhibitor

5980. The method of item 5880 wherein the agent is a tubulin antagonist

5981. The method of item 5880 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-11 0 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

5982. The method of item 5880 wherein the agent is a VEGF inhibitor.

5983. The method of item 5880 wherein the agent is a vitamin D receptor agonist.

5984. The method of item 5880 wherein the agent is ZD-6474 (an angiogenesis inhibitor).

5985. The method of item 5880 wherein the agent is AP-23573 (an mTOR inhibitor).

5986. The method of item 5880 wherein the agent is synthadotin (a tubulin antagonist).

5987. The method of item 5880 wherein the agent is S-0885 (a collagenase inhibitor).

5988. The method of item 5880 wherein the agent is aplidine (an elongation factor-1 alpha inhibitor).

5989. The method of item 5880 wherein the agent is ixabepilone (an epithilone).

5990. The method of item 5880 wherein the agent is IDN-5390 (an angiogenesis inhibitor).

5991. The method of item 5880 wherein the agent is SB-2723005 (an angiogenesis inhibitor).

5992. The method of item 5880 wherein the agent is ABT-518 (an angiogenesis inhibitor).

5993. The method of item 5880 wherein the agent is combretastatin (an angiogenesis inhibitor).

5994. The method of item 5880 wherein the agent is anecortave acetate (an angiogenesis inhibitor).

5995. The method of item 5880 wherein the agent is SB-715992 (a kinesin antagonist).

5996. The method of item 5880 wherein the agent is temsirolimus (an mTOR inhibitor).

5997. The method of item 5880 wherein the agent is adalimumab (a TNFα antagonist).

5998. The method of item 5880, wherein the composition comprises a polymer.

5999. The method of item 5880, wherein the composition comprises a polymer, and the polymer is, or comprises, a copolymer.

6000. The method of item 5880, wherein the composition comprises a polymer, and the polymer is, or comprises, a block copolymer.

6001. The method of item 5880, wherein the composition comprises a polymer, and the polymer is, or comprises, a random copolymer.

6002. The method of item 5880, wherein the composition comprises a polymer, and the polymer is, or comprises, a biodegradable polymer.

6003. The method of item 5880, wherein the composition comprises a polymer, and the polymer is, or comprises, a non-biodegradable polymer.

6004. The method of item 5880, wherein the composition comprises a polymer, and the polymer is, or comprises, a hydrophilic polymer.

6005. The method of item 5880, wherein the composition comprises a polymer, and the polymer is, or comprises, a hydrophobic polymer.

6006. The method of item 5880, wherein the composition comprises a polymer, and the polymer is, or comprises, a polymer having hydrophilic domains.

6007. The method of item 5880, wherein the composition comprises a polymer, and the polymer is, or comprises, a polymer having hydrophobic domains.

6008. The method of item 5880, wherein the composition comprises a polymer, and the polymer is, or comprises, a non-conductive polymer.

6009. The method of item 5880, wherein the composition comprises a polymer, and the polymer is, or comprises, an elastomer.

6010. The method of item 5880, wherein the composition comprises a polymer, and the polymer is, or comprises, a hydrogel.

6011. The method of item 5880, wherein the composition comprises a polymer, and the polymer is, or comprises, a silicone polymer.

6012. The method of item 5880, wherein the composition comprises a polymer, and the polymer is, or comprises, a hydrocarbon polymer.

6013. The method of item 5880, wherein the composition comprises a polymer, and the polymer is, or comprises, a styrene-derived polymer.

6014. The method of item 5880, wherein the composition comprises a polymer, and the polymer is, or comprises, a butadiene-derived polymer.

6015. The method of item 5880, wherein the composition comprises a polymer, and the polymer is, or comprises, a macromer.

6016. The method of item 5880, wherein the composition comprises a polymer, and the polymer is, or comprises, a poly(ethylene glycol) polymer.

6017. The method of item 5880, wherein the composition comprises a polymer, and the polymer is, or comprises, an amorphous polymer.

6018. The method of item 5880, wherein the composition further comprises a second pharmaceutically active agent.

6019. The method of item 5880, wherein the composition further comprises an anti-inflammatory agent.

6020. The method of item 5880, wherein the composition further comprises an agent that inhibits infection.

6021. The method of item 5880, wherein the composition further comprises an anthracycline.

6022. The method of item 5880, wherein the composition further comprises doxorubicin.

6023. The method of item 5880 wherein the composition further comprises mitoxantrone.

6024. The method of item 5880 wherein the composition further comprises a fluoropyrimidine.

6025. The method of item 5880, wherein the composition further comprises 5-fluorouracil (5-FU).

6026. The method of item 5880, wherein the composition further comprises a folic acid antagonist.

6027. The method of item 5880, wherein the composition further comprises methotrexate.

6028. The method of item 5880, wherein the composition further comprises a podophylotoxin.

6029. The method of item 5880, wherein the composition further comprises etoposide.

6030. The method of item 5880, wherein the composition further comprises camptothecin.

6031. The method of item 5880, wherein the composition further comprises a hydroxyurea.

6032. The method of item 5880, wherein the composition further comprises a platinum complex.

6033. The method of item 5880, wherein the composition further comprises cisplatin.

6034. The method of item 5880 wherein the composition further comprises an anti-thrombotic agent.

6035. The method of item 5880, wherein the composition further comprises a visualization agent.

6036. The method of item 5880, wherein the composition further comprises a visualization agent, and the visualization agent is a radiopaque material, wherein the radiopaque material comprises a metal, a halogenated compound, or a barium containing compound

6037. The method of item 5880, wherein the composition further comprises a visualization agent, and the visualization agent is, or comprises, barium, tantalum, or technetium.

6038. The method of item 5880, wherein the composition further comprises a visualization agent, and the visualization agent is, or comprises, an MRI responsive material.

6039. The method of item 5880, wherein the composition further comprises a visualization agent, and the visualization agent is, or comprises, a gadolinium chelate.

6040. The method of item 5880, wherein the composition further comprises a visualization agent, and the visualization agent is, or comprises, iron, magnesium, manganese, copper, or chromium.

6041. The method of item 5880, wherein the composition further comprises a visualization agent, and the visualization agent is, or comprises, iron oxide compound.

6042. The method of item 5880, wherein the composition further comprises a visualization agent, and the visualization agent is, or comprises, a dye, pigment, or colorant.

6043. The method of item 5880 wherein the agent is released in effective concentrations from the composition comprising the agent by diffusion over a period ranging from the time of administration to about 90 days.

6044. The method of item 5880 wherein the agent is released in effective concentrations from the composition comprising the agent by erosion of the composition over a period ranging from the time of administration to about 90 days.

6045. The method of item 5880 wherein the composition further comprises an inflammatory cytokine.

6046. The method of item 5880 wherein the composition further comprises an agent that stimulates cell proliferation.

6047. The method of item 5880 wherein the composition further comprises a polymeric carrier.

6048. The method of item 5880 wherein the composition is in the form of a gel, paste, or spray.

6049. The method of item 5880 wherein the electrical device is partially constructed with the agent or the composition.

6050. The method of item 5880 wherein the electrical device is impregnated with the agent or the composition.

6051. The method of item 5880, wherein the agent or the composition forms a coating, and the coating directly contacts the electrical device.

6052. The method of item 5880, wherein the agent or the composition forms a coating, and the coating indirectly contacts the electrical device.

6053. The method of item 5880 wherein the agent or the composition forms a coating, and the coating partially covers the electrical device.

6054. The method of item 5880, wherein the agent or the composition forms a coating, and the coating completely covers the electrical device.

6055. The method of item 5880 wherein the agent or the composition is located within pores or holes of the electrical device.

6056. The method of item 5880 wherein the agent or the composition is located within a channel, lumen, or divet of the electrical device.

6057. The method of item 5880 wherein the electrical device further comprises an echogenic material.

6058. The method of item 5880 wherein the electrical device further comprises an echogenic material, wherein the echogenic material is in the form of a coating.

6059. The method of item 5880 wherein the electrical device is sterile.

6060. The method of item 5880 wherein the agent is delivered from the electrical device, wherein the agent is released into tissue in the vicinity of the electrical device after deployment of the electrical device.

6061. The method of item 5880 wherein the agent is delivered from the electrical device, wherein the agent is released into tissue in the vicinity of the electrical device after deployment of the electrical device, wherein the tissue is connective tissue.

6062. The method of item 5880 wherein the agent is delivered from the electrical device, wherein the agent is released into tissue in the vicinity of the electrical device after deployment of the electrical device, wherein the tissue is muscle tissue.

6063. The method of item 5880 wherein the agent is delivered from the electrical device, wherein the agent is released into tissue in the vicinity of the electrical device after deployment of the electrical device, wherein the tissue is nerve tissue.

6064. The method of item 5880 wherein the agent is delivered from the electrical device, wherein the agent is released into tissue in the vicinity of the electrical device after deployment of the electrical device, wherein the tissue is epithelium tissue.

6065. The method of item 5880 wherein the agent is delivered from the electrical device, wherein the agent is released in effective concentrations from the electrical device over a period ranging from the time of deployment of the electrical device to about 1 year.

6066. The method of item 5880 wherein the agent is delivered from the electrical device, wherein the agent is released in effective concentrations from the electrical device over a period ranging from about 1 month to 6 months.

6067. The method of item 5880 wherein the agent is delivered from the electrical device, wherein the agent is released in effective concentrations from the electrical device over a period ranging from about 1-90 days.

6068. The method of item 5880 wherein the agent is delivered from the electrical device, wherein the agent is released in effective concentrations from the electrical device at a constant rate.

6069. The method of item 5880 wherein the agent is delivered from the electrical device, wherein the agent is released in effective concentrations from the electrical device at an increasing rate.

6070. The method of item 5880 wherein the agent is delivered from the electrical device, wherein the agent is released in effective concentrations from the electrical device at a decreasing rate.

6071. The method of item 5880 wherein the agent is delivered from the electrical device, wherein the electrical device comprises about 0.01 μg to about 10 μg of the agent.

6072. The method of item 5880 wherein the agent is delivered from the electrical device, wherein the electrical device comprises about 10 μg to about 10 mg of the agent.

6073. The method of item 5880 wherein the agent is delivered from the electrical device, wherein the electrical device comprises about 10 mg to about 250 mg of the agent.

6074. The method of item 5880 wherein the agent is delivered from the electrical device, wherein the electrical device comprises about 250 mg to about 1000 mg of the agent.

6075. The method of item 5880 wherein the agent is delivered from the electrical device, wherein the electrical device comprises about 1000 mg to about 2500 mg of the agent.

6076. The method of item 5880 wherein the agent is delivered from the electrical device, wherein a surface of the electrical device comprises less than 0.01 μg of the agent per mm² of electrical device surface to which the agent is applied.

6077. The method of item 5880 wherein the agent is delivered from the electrical device, wherein a surface of the electrical device comprises about 0.01 μg to about 1 μg of the agent per mm² of electrical device surface to which the agent is applied.

6078. The method of item 5880 wherein the agent is delivered from the electrical device, wherein a surface of the electrical device comprises about 1 μg to about 10 μg of the agent per mm² of electrical device surface to which the agent is applied.

6079. The method of item 5880 wherein the agent is delivered from the electrical device, wherein a surface of the electrical device comprises about 10 μg to about 250 μg of the agent per mm² of electrical device surface to which the agent is applied.

6080. The method of item 5880 wherein the agent is delivered from the electrical device, wherein a surface of the electrical device comprises about 250 μg to about 1000 μg of the agent per mm² of electrical device surface to which the agent is applied.

6081. The method of item 5880 wherein the agent is delivered from the electrical device, wherein a surface of the electrical device comprises about 1000 μg to about 2500 μg of the agent per mm² of electrical device surface to which the agent is applied.

6082. The method of item 5880, wherein the electrical device further comprises a coating, and the coating is a uniform coating.

6083. The method of item 5880, wherein the electrical device further comprises a coating, and the coating is a non-uniform coating.

6084. The method of item 5880, wherein the electrical device further comprises a coating, and the coating is a discontinuous coating.

6085. The method of item 5880, wherein the electrical device further comprises a coating, and the coating is a patterned coating.

6086. The method of item 5880, wherein the electrical device further comprises a coating, and the coating has a thickness of 100 μm or less.

6087. The method of item 5880, wherein the electrical device further comprises a coating, and the coating has a thickness of 10 μm or less.

6088. The method of item 5880, wherein the electrical device further comprises a coating, and the coating adheres to the surface of the electrical device upon deployment of the electrical device.

6089. The method of item 5880, wherein the electrical device further comprises a coating, and the coating is stable at room temperature for a period of at least 1 year.

6090. The method of item 5880, wherein the electrical device further comprises a coating, and the agent is present in the coating in an amount ranging between about 0.0001% to about 1% by weight.

6091. The method of item 5880, wherein the electrical device further comprises a coating, and the agent is present in the coating in an amount ranging between about 1% to about 10% by weight.

6092. The method of item 5880, wherein the electrical device further comprises a coating, and the agent is present in the coating in an amount ranging between about 10% to about 25% by weight.

6093. The method of item 5880, wherein the electrical device further comprises a coating, and the agent is present in the coating in an amount ranging between about 25% to about 70% by weight.

6094. The method of item 5880, wherein the electrical device further comprises a coating, and the coating comprises a polymer.

6095. The method of item 5880, wherein the electrical device comprises a first coating having a first composition and a second coating having a second composition.

6096. The method of item 5880, wherein the electrical device comprises a first coating having a first composition and a second coating having a second composition, wherein the first composition and the second composition are different.

6097. The method of item 5880 wherein the agent or the composition is affixed to the electrical device.

6098. The method of item 5880 wherein the agent or the composition is covalently attached to the electrical device.

6099. The method of item 5880 wherein the agent or the composition is non-covalently attached to the electrical device.

6100. The method of item 5880 wherein the electrical device comprises a coating that absorbs the agent or the composition.

6101. The method of item 5880 wherein the electrical device is interweaved with a thread composed of, or coated with, the agent or the composition.

6102. The method of item 5880 wherein a portion of the electrical device is covered with a sleeve that contains the agent or the composition.

6103. The method of item 5880 wherein the electrical device is completely covered with a sleeve that contains the agent or the composition.

6104. The method of item 5880 wherein a portion of the electrical device is covered with a mesh that contains the agent or the composition.

6105. The method of item 5880 wherein the electrical device is completely covered with a mesh that contains the agent or the composition.

6106. The method of item 5880 wherein the agent or the composition is applied to the electrical device surface prior to the placing of the electrical device into the host.

6107. The method of item 5880 wherein the agent or the composition is applied to the electrical device surface during the placing of the electrical device into the host

6108. The method of item 5880 wherein the agent or the composition is applied to the electrical device surface immediately after the placing of the electrical device into the host.

6109. The method of item 5880 wherein the agent or the composition is applied to the surface of the tissue in the host surrounding the electrical device prior to the placing of the electrical device into the host.

6110. The method of item 5880 wherein the agent or the composition is applied to the surface of the tissue in the host surrounding the electrical device during the placing of the electrical device into the host.

6111. The method of item 5880 wherein the agent or the composition is applied to the surface of the tissue in the host surrounding the electrical device immediately after the placing of the electrical device into the host.

6112. The method of item 5880 wherein the agent or the composition is topically applied into the anatomical space where the electrical device is placed.

6113. The method of item 5880 wherein the agent or the composition is percutaneously injected into the tissue in the host surrounding the electrical device.

6114. The method for inhibiting scarring of any one of items 5880-6113 wherein the cardiac pacemaker is an adaptive rate pacemaker.

6115. The method for inhibiting scarring of any one of items 5880-6113 wherein the cardiac pacemaker is a rate responsive pacemaker.

6116. A method for inhibiting scarring comprising placing an implantable cardioverter defibrillator (ICD) system (i.e., an electrical device) and an anti-scarring agent or a composition comprising an ant-scarring agent into an animal host, wherein the agent inhibits scarring.

6117. The method of item 6116 wherein the agent is an adensosine A2A receptor antagonist.

6118. The method of item 6116 wherein the agent is an AKT inhibitor.

6119. The method of item 6116 wherein the agent is an alpha 2 integrin antagonist, wherein the alpha 2 integrin antagonist is Pharmaprojects No. 5754 (Merck KgaA).

6120. The method of item 6116 wherein the agent is an alpha 4 integrin antagonist.

6121. The method of item 6116 wherein the agent is an alpha 7 nicotinic receptor agonist.

6122. The method of item 6116 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

6123. The method of item 6116 wherein the agent is an apoptosis antagonist.

6124. The method of item 6116 wherein the agent is an apoptosis activator.

6125. The method of item 6116 wherein the agent is a beta 1 integrin antagonist.

6126. The method of item 6116 wherein the agent is a beta tubulin inhibitor.

6127. The method of item 6116 wherein the agent is a blocker of enzyme production in Hepatitis C.

6128. The method of item 6116 wherein the agent is a Bruton's tyrosine kinase inhibitor.

6129. The method of item 6116 wherein the agent is a calcineurin inhibitor.

6130. The method of item 6116 wherein the agent is a caspase 3 inhibitor.

6131. The method of item 6116 wherein the agent is a CC chemokine receptor antagonist.

6132. The method of item 6116 wherein the agent is a cell cycle inhibitor selected from the group consisting of SNS-595 (Sunesis), synthadotin, KRX-0403, and an analogue or derivative thereof.

6133. The method of item 6116 wherein the agent is a cathepsin B inhibitor.

6134. The method of item 6116 wherein the agent is a cathepsin K inhibitor, wherein the cathepsin K inhibitor is 462795 (GlaxoSmithKline), INPL-022-D6 (Amura Therapeutics), or an analogue or derivative thereof.

6135. The method of item 6116 wherein the agent is a cathepsin L inhibitor.

6136. The method of item 6116 wherein the agent is a CD40 antagonist.

6137. The method of item 6116 wherein the agent is a chemokine receptor agonist.

6138. The method of item 6116 wherein the agent is a chymase inhibitor.

6139. The method of item 6116 wherein the agent is a collagenase antagonist.

6140. The method of item 6116 wherein the agent is a CXCR antagonist.

6141. The method of item 6116 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

6142. The method of item 6116 wherein the agent is a cyclooxygenase 1 inhibitor.

6143. The method of item 6116 wherein the agent is a DHFR inhibitor.

6144. The method of item 6116 wherein the agent is a dual integrin inhibitor.

6145. The method of item 6116 wherein the agent is an elastase inhibitor.

6146. The method of item 6116 wherein the agent is an elongation factor-1 alpha inhibitor.

6147. The method of item 6116 wherein the agent is an endothelial growth factor antagonist.

6148. The method of item 6116 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

6149. The method of item 6116 wherein the agent is an endotoxin antagonist.

6150. The method of item 6116 wherein the agent is an epothilone and tubulin binder.

6151. The method of item 6116 wherein the agent is an estrogen receptor antagonist.

6152. The method of item 6116 wherein the agent is an FGF inhibitor.

6153. The method of item 6116 wherein the agent is a farnexyl transferase inhibitor.

6154. The method of item 6116 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

6155. The method of item 6116 wherein the agent is an FLT-3 kinase inhibitor.

6156. The method of item 6116 wherein the agent is an FGF receptor kinase inhibitor.

6157. The method of item 6116 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

6158. The method of item 6116 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

6159. The method of item 6116 wherein the agent is a histone deacetylase inhibitor.

6160. The method of item 6116 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

6161. The method of item 6116 wherein the agent is an ICAM inhibitor.

6162. The method of item 6116 wherein the agent is an IL, ICE and IRAK antagonist, wherein the antagonist is a CJ-14877, CP-424174 (Pfizer), NF-61 (Negma-Lerads), and an analogue or derivative thereof.

6163. The method of item 6116 wherein the agent is an IL-2 inhibitor.

6164. The method of item 6116 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

6165. The method of item 6116 wherein the agent is an IMPDH (inosine monophosphate).

6166. The method of item 6116 wherein the agent is an integrin antagonist.

6167. The method of item 6116 wherein the agent is an interleukin antagonist.

6168. The method of item 6116 wherein the agent is an inhibitor of type III receptor tyrosine kinase.

6169. The method of item 6116 wherein the agent is an irreversible inhibitor of enzyme methionine aminopeptidase type 2.

6170. The method of item 6116 wherein the agent is an isozyme selective delta protein kinase C inhibitor.

6171. The method of item 6116 wherein the agent a JAK3 enzyme inhibitor.

6172. The method of item 6116 wherein the agent is a JNK inhibitor.

6173. The method of item 6116 wherein the agent is a kinase inhibitor.

6174. The method of item 6116 wherein the agent is kinesin antagonist.

6175. The method of item 6116 wherein the agent is a kinesin antagonist.

6176. The method of item 6116 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

6177. The method of item 6116 wherein the agent is an MAP kinase inhibitor.

6178. The method of item 6116 wherein the agent is a matrix metalloproteinase inhibitor.

6179. The method of item 6116 wherein the agent is an MCP-CCR2 inhibitor.

6180. The method of item 6116 wherein the agent is an mTOR inhibitor.

6181. The method of item 6116 wherein the agent is an mTOR kinase inhibitor.

6182. The method of item 6116 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

6183. The method of item 6116 wherein the agent is an MIF inhibitor.

6184. The method of item 6116 wherein the agent is an MMP inhibitor.

6185. The method of item 6116 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

6186. The method of item 6116 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

6187. The method of item 6116 wherein the agent is a nitric oxide agonist.

6188. The method of item 6116 wherein the agent is an ornithine decarboxylase inhibitor.

6189. The method of item 6116 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

6190. The method of item 6116 wherein the agent is a palmitoyl-protein thioesterase inhibitor.

6191. The method of item 6116 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

6192. The method of item 6116 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

6193. The method of item 6116 wherein the agent is a phosphatase inhibitor.

6194. The method of item 6116 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

6195. The method of item 6116 wherein the agent is a PKC inhibitor.

6196. The method of item 6116 wherein the agent is a platelet activating factor antagonist.

6197. The method of item 6116 wherein the agent is a platelet-derived growth factor receptor kinase inhibitor.

6198. The method of item 6116 wherein the agent is a prolyl hydroxylase inhibitor.

6199. The method of item 6116 wherein the agent is a polymorphonuclear neutrophil inhibitor.

6200. The method of item 6116 wherein the agent is a protein kinase B inhibitor.

6201. The method of item 6116 wherein the agent is a protein kinase C stimulant.

6202. The method of item 6116 wherein the agent is a purine nucleoside analogue.

6203. The method of item 6116 wherein the agent is a purinoreceptor P2X antagonist.

6204. The method of item 6116 wherein the agent is a Raf kinase inhibitor.

6205. The method of item 6116 wherein the agent is a reversible inhibitor of ErbB1 and ErbB2.

6206. The method of item 6116 wherein the agent is a ribonucleoside triphosphate reductase inhibitor.

6207. The method of item 6116 wherein the agent is an SDF-1 antagonist.

6208. The method of item 6116 wherein the agent is a sheddase inhibitor.

6209. The method of item 6116 wherein the agent is an SRC inhibitor.

6210. The method of item 6116 wherein the agent is a stromelysin inhibitor.

6211. The method of item 6116 wherein the agent is an Syk kinase inhibitor.

6212. The method of item 6116 wherein the agent is a telomerase inhibitor.

6213. The method of item 6116 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

6214. The method of item 6116 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TN F-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

6215. The method of item 6116 wherein the agent is a Toll receptor inhibitor.

6216. The method of item 6116 wherein the agent is a tubulin antagonist.

6217. The method of item 6116 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

6218. The method of item 6116 wherein the agent is a VEGF inhibitor.

6219. The method of item 6116 wherein the agent is a vitamin D receptor agonist.

6220. The method of item 6116 wherein the agent is ZD-6474 (an angiogenesis inhibitor).

6221. The method of item 6116 wherein the agent is AP-23573 (an mTOR inhibitor).

6222. The method of item 6116 wherein the agent is synthadotin (a tubulin antagonist).

6223. The method of item 6116 wherein the agent is S-0885 (a collagenase inhibitor).

6224. The method of item 6116 wherein the agent is aplidine (an elongation factor-1 alpha inhibitor).

6225. The method of item 6116 wherein the agent is ixabepilone (an epithilone).

6226. The method of item 6116 wherein the agent is IDN-5390 (an angiogenesis inhibitor).

6227. The method of item 6116 wherein the agent is SB-2723005 (an angiogenesis inhibitor).

6228. The method of item 6116 wherein the agent is ABT-518 (an angiogenesis inhibitor).

6229. The method of item 6116 wherein the agent is combretastatin (an angiogenesis inhibitor).

6230. The method of item 6116 wherein the agent is anecortave acetate (an angiogenesis inhibitor).

6231. The method of item 6116 wherein the agent is SB-715992 (a kinesin antagonist).

6232. The method of item 6116 wherein the agent is temsirolimus (an mTOR inhibitor).

6233. The method of item 6116 wherein the agent is adalimumab (a TNFα antagonist).

6234. The method of item 6116, wherein the composition comprises a polymer.

6235. The method of item 6116, wherein the composition comprises a polymer, and the polymer is, or comprises, a copolymer.

6236. The method of item 6116, wherein the composition comprises a polymer, and the polymer is, or comprises, a block copolymer.

6237. The method of item 6116, wherein the composition comprises a polymer, and the polymer is, or comprises, a random copolymer.

6238. The method of item 6116, wherein the composition comprises a polymer, and the polymer is, or comprises, a biodegradable polymer.

6239. The method of item 6116, wherein the composition comprises a polymer, and the polymer is, or comprises, a non-biodegradable polymer.

6240. The method of item 6116, wherein the composition comprises a polymer, and the polymer is, or comprises, a hydrophilic polymer.

6241. The method of item 6116, wherein the composition comprises a polymer, and the polymer is, or comprises, a hydrophobic polymer.

6242. The method of item 6116, wherein the composition comprises a polymer, and the polymer is, or comprises, a polymer having hydrophilic domains.

6243. The method of item 6116, wherein the composition comprises a polymer, and the polymer is, or comprises, a polymer having hydrophobic domains.

6244. The method of item 6116, wherein the composition comprises a polymer, and the polymer is, or comprises, a non-conductive polymer.

6245. The method of item 6116, wherein the composition comprises a polymer, and the polymer is, or comprises, an elastomer.

6246. The method of item 6116, wherein the composition comprises a polymer, and the polymer is, or comprises, a hydrogel.

6247. The method of item 6116, wherein the composition comprises a polymer, and the polymer is, or comprises, a silicone polymer.

6248. The method of item 6116, wherein the composition comprises a polymer, and the polymer is, or comprises, a hydrocarbon polymer.

6249. The method of item 6116, wherein the composition comprises a polymer, and the polymer is, or comprises, a styrene-derived polymer.

6250. The method of item 6116, wherein the composition comprises a polymer, and the polymer is, or comprises, a butadiene-derived polymer.

6251. The method of item 6116, wherein the composition comprises a polymer, and the polymer is, or comprises, a macromer.

6252. The method of item 6116, wherein the composition comprises a polymer, and the polymer is, or comprises, a poly(ethylene glycol) polymer.

6253. The method of item 6116, wherein the composition comprises a polymer, and the polymer is, or comprises, an amorphous polymer.

6254. The method of item 6116, wherein the composition further comprises a second pharmaceutically active agent.

6255. The method of item 6116, wherein the composition further comprises an anti-inflammatory agent.

6256. The method of item 6116, wherein the composition further comprises an agent that inhibits infection.

6257. The method of item 6116, wherein the composition further comprises an anthracycline.

6258. The method of item 6116, wherein the composition further comprises doxorubicin.

6259. The method of item 6116 wherein the composition further comprises mitoxantrone.

6260. The method of item 6116 wherein the composition further comprises a fluoropyrimidine.

6261. The method of item 6116, wherein the composition further comprises 5-fluorouracil (5-FU).

6262. The method of item 6116, wherein the composition further comprises a folic acid antagonist.

6263. The method of item 6116, wherein the composition further comprises methotrexate.

6264. The method of item 6116, wherein the composition further comprises a podophylotoxin.

6265. The method of item 6116, wherein the composition further comprises etoposide.

6266. The method of item 6116, wherein the composition further comprises camptothecin.

6267. The method of item 6116, wherein the composition further comprises a hydroxyurea.

6268. The method of item 6116, wherein the composition further comprises a platinum complex.

6269. The method of item 6116, wherein the composition further comprises cisplatin.

6270. The method of item 6116 wherein the composition further comprises an anti-thrombotic agent.

6271. The method of item 6116, wherein the composition further comprises a visualization agent.

6272. The method of item 6116, wherein the composition further comprises a visualization agent, and the visualization agent is a radiopaque material, wherein the radiopaque material comprises a metal, a halogenated compound, or a barium containing compound.

6273. The method of item 6116, wherein the composition further comprises a visualization agent, and the visualization agent is, or comprises, barium, tantalum, or technetium.

6274. The method of item 6116, wherein the composition further comprises a visualization agent, and the visualization agent is, or comprises, an MRI responsive material.

6275. The method of item 6116, wherein the composition further comprises a visualization agent, and the visualization agent is, or comprises, a gadolinium chelate.

6276. The method of item 6116, wherein the composition further comprises a visualization agent, and the visualization agent is, or comprises, iron, magnesium, manganese, copper, or chromium.

6277. The method of item 6116, wherein the composition further comprises a visualization agent, and the visualization agent is, or comprises, iron oxide compound.

6278. The method of item 6116, wherein the composition further comprises a visualization agent, and the visualization agent is, or comprises, a dye, pigment, or colorant.

6279. The method of item 6116 wherein the agent is released in effective concentrations from the composition comprising the agent by diffusion over a period ranging from the time of administration to about 90 days.

6280. The method of item 6116 wherein the agent is released in effective concentrations from the composition comprising the agent by erosion of the composition over a period ranging from the time of administration to about 90 days.

6281. The method of item 6116 wherein the composition further comprises an inflammatory cytokine.

6282. The method of item 6116 wherein the composition further comprises an agent that stimulates cell proliferation.

6283. The method of item 6116 wherein the composition further comprises a polymeric carrier.

6284. The method of item 6116 wherein the composition is in the form of a gel, paste, or spray.

6285. The method of item 6116 wherein the electrical device is partially constructed with the agent or the composition.

6286. The method of item 6116 wherein the electrical device is impregnated with the agent or the composition.

6287. The method of item 6116, wherein the agent or the composition forms a coating, and the coating directly contacts the electrical device.

6288. The method of item 6116, wherein the agent or the composition forms a coating, and the coating indirectly contacts the electrical device.

6289. The method of item 6116 wherein the agent or the composition forms a coating, and the coating partially covers the electrical device.

6290. The method of item 6116, wherein the agent or the composition forms a coating, and the coating completely covers the electrical device.

6291. The method of item 6116 wherein the agent or the composition is located within pores or holes of the electrical device.

6292. The method of item 6116 wherein the agent or the composition is located within a channel, lumen, or divet of the electrical device.

6293. The method of item 6116 wherein the electrical device further comprises an echogenic material.

6294. The method of item 6116 wherein the electrical device further comprises an echogenic material, wherein the echogenic material is in the form of a coating.

6295. The method of item 6116 wherein the electrical device is sterile.

6296. The method of item 6116 wherein the agent is delivered from the electrical device, wherein the agent is released into tissue in the vicinity of the electrical device after deployment of the electrical device.

6297. The method of item 6116 wherein the agent is delivered from the electrical device, wherein the agent is released into tissue in the vicinity of the electrical device after deployment of the electrical device, wherein the tissue is connective tissue

6298. The method of item 6116 wherein the agent is delivered from the electrical device, wherein the agent is released into tissue in the vicinity of the electrical device after deployment of the electrical device, wherein the tissue is muscle tissue.

6299. The method of item 6116 wherein the agent is delivered from the electrical device, wherein the agent is released into tissue in the vicinity of the electrical device after deployment of the electrical device, wherein the tissue is nerve tissue.

6300. The method of item 6116 wherein the agent is delivered from the electrical device, wherein the agent is released into tissue in the vicinity of the electrical device after deployment of the electrical device, wherein the tissue is epithelium tissue.

6301. The method of item 6116 wherein the agent is delivered from the electrical device, wherein the agent is released in effective concentrations from the electrical device over a period ranging from the time of deployment of the electrical device to about 1 year.

6302. The method of item 6116 wherein the agent is delivered from the electrical device, wherein the agent is released in effective concentrations from the electrical device over a period ranging from about 1 month to 6 months.

6303. The method of item 6116 wherein the agent is delivered from the electrical device, wherein the agent is released in effective concentrations from the electrical device over a period ranging from about 1-90 days.

6304. The method of item 6116 wherein the agent is delivered from the electrical device, wherein the agent is released in effective concentrations from the electrical device at a constant rate.

6305. The method of item 6116 wherein the agent is delivered from the electrical device, wherein the agent is released in effective concentrations from the electrical device at an increasing rate.

6306. The method of item 6116 wherein the agent is delivered from the electrical device, wherein the agent is released in effective concentrations from the electrical device at a decreasing rate.

6307. The method of item 6116 wherein the agent is delivered from the electrical device, wherein the electrical device comprises about 0.01 μg to about 10 μg of the agent.

6308. The method of item 6116 wherein the agent is delivered from the electrical device, wherein the electrical device comprises about 10 pg to about 10 mg of the agent.

6309. The method of item 6116 wherein the agent is delivered from the electrical device, wherein the electrical device comprises about 10 mg to about 250 mg of the agent.

6310. The method of item 6116 wherein the agent is delivered from the electrical device, wherein the electrical device comprises about 250 mg to about 1000 mg of the agent.

6311. The method of item 6116 wherein the agent is delivered from the electrical device, wherein the electrical device comprises about 1000 mg to about 2500 mg of the agent.

6312. The method of item 6116 wherein the agent is delivered from the electrical device, wherein a surface of the electrical device comprises less than 0.01 μg of the agent per mm² of electrical device surface to which the agent is applied.

6313. The method of item 6116 wherein the agent is delivered from the electrical device, wherein a surface of the electrical device comprises about 0.01 μg to about 1 μg of the agent per mm² of electrical device surface to which the agent is applied.

6314. The method of item 6116 wherein the agent is delivered from the electrical device, wherein a surface of the electrical device comprises about 1 μg to about 10 μg of the agent per mm² of electrical device surface to which the agent is applied.

6315. The method of item 6116 wherein the agent is delivered from the electrical device, wherein a surface of the electrical device comprises about 10 μg to about 250 μg of the agent per mm² of electrical device surface to which the agent is applied.

6316. The method of item 6116 wherein the agent is delivered from the electrical device, wherein a surface of the electrical device comprises about 250 μg to about 1000 μg of the agent per mm² of electrical device surface to which the agent is applied.

6317. The method of item 6116 wherein the agent is delivered from the electrical device, wherein a surface of the electrical device comprises about 1000 μg to about 2500 μg of the agent per mm² of electrical device surface to which the agent is applied.

6318. The method of item 6116, wherein the electrical device further comprises a coating, and the coating is a uniform coating.

6319. The method of item 6116, wherein the electrical device further comprises a coating, and the coating is a non-uniform coating.

6320. The method of item 6116, wherein the electrical device further comprises a coating, and the coating is a discontinuous coating.

6321. The method of item 6116, wherein the electrical device further comprises a coating, and the coating is a patterned coating.

6322. The method of item 6116, wherein the electrical device further comprises a coating, and the coating has a thickness of 100 μm or less.

6323. The method of item 6116, wherein the electrical device further comprises a coating, and the coating has a thickness of 10 μm or less.

6324. The method of item 6116, wherein the electrical device further comprises a coating, and the coating adheres to the surface of the electrical device upon deployment of the electrical device.

6325. The method of item 6116, wherein the electrical device further comprises a coating, and the coating is stable at room temperature for a period of at least 1 year.

6326. The method of item 6116, wherein the electrical device further comprises a coating, and the agent is present in the coating in an amount ranging between about 0.0001% to about 1% by weight.

6327. The method of item 6116, wherein the electrical device further comprises a coating, and the agent is present in the coating in an amount ranging between about 1% to about 10% by weight.

6328. The method of item 6116, wherein the electrical device further comprises a coating, and the agent is present in the coating in an amount ranging between about 10% to about 25% by weight.

6329. The method of item 6116, wherein the electrical device further comprises a coating, and the agent is present in the coating in an amount ranging between about 25% to about 70% by weight.

6330. The method of item 6116, wherein the electrical device further comprises a coating, and the coating comprises a polymer.

6331. The method of item 6116, wherein the electrical device comprises a first coating having a first composition and a second coating having a second composition.

6332. The method of item 6116, wherein the electrical device comprises a first coating having a first composition and a second coating having a second composition, wherein the first composition and the second composition are different.

6333. The method of item 6116 wherein the agent or the composition is affixed to the electrical device.

6334. The method of item 6116 wherein the agent or the composition is covalently attached to the electrical device.

6335. The method of item 6116 wherein the agent or the composition is non-covalently attached to the electrical device.

6336. The method of item 6116 wherein the electrical device comprises a coating that absorbs the agent or the composition.

6337. The method of item 6116 wherein the electrical device is interweaved with a thread composed of, or coated with, the agent or the composition.

6338. The method of item 6116 wherein a portion of the electrical device is covered with a sleeve that contains the agent or the composition.

6339. The method of item 6116 wherein the electrical device is completely covered with a sleeve that contains the agent or the composition.

6340. The method of item 6116 wherein a portion of the electrical device is covered with a mesh that contains the agent or the composition.

6341. The method of item 6116 wherein the electrical device is completely covered with a mesh that contains the agent or the composition.

6342. The method of item 6116 wherein the agent or the composition is applied to the electrical device surface prior to the placing of the electrical device into the host.

6343. The method of item 6116 wherein the agent or the composition is applied to the electrical device surface during the placing of the electrical device into the host.

6344. The method of item 6116 wherein the agent or the composition is applied to the electrical device surface immediately after the placing of the electrical device into the host

6345. The method of item 6116 wherein the agent or the composition is applied to the surface of the tissue in the host surrounding the electrical device prior to the placing of the electrical device into the host.

6346. The method of item 6116 wherein the agent or the composition is applied to the surface of the tissue in the host surrounding the electrical device during the placing of the electrical device into the host.

6347. The method of item 6116 wherein the agent or the composition is applied to the surface of the tissue in the host surrounding the electrical device immediately after the placing of the electrical device into the host.

6348. The method of item 6116 wherein the agent or the composition is topically applied into the anatomical space where the electrical device is placed.

6349. The method of item 6116 wherein the agent or the composition is percutaneously injected into the tissue in the host surrounding the electrical device.

6350. The method for inhibiting scarring of any one of items 6116-6349 wherein the implantable cardioverter defibrillator is adapted for treating tachyarraythmias.

6351. The method for inhibiting scarring of any one of items 6116-6349 wherein the implantable cardioverter defibrillator is adapted for ventricular tachycardia.

6352. The method for inhibiting scarring of any one of items 6116-6349 wherein the implantable cardioverter defibrillator is adapted for treating ventricular fibrillation.

6353. The method for inhibiting scarring of any one of items 6116-6349 wherein the implantable cardioverter defibrillator is adapted for treating atrial tachycardia.

6354. The method for inhibiting scarring of any one of items 6116-6349 wherein the implantable cardioverter defibrillator is adapted for treating atrial fibrillation.

6355. The method for inhibiting scarring of any one of items 6116-6349 wherein the implantable cardioverter defibrillator is adapted for treating arrhythmias.

6356. A method for inhibiting scarring comprising placing a vagus nerve stimulator for treating arrhythemia (i.e., an electrical device) and an anti-scarring agent or a composition comprising an ant-scarring agent into an animal host, wherein the agent inhibits scarring.

6357. The method of item 6356 wherein the agent is an adensosine A2A receptor antagonist.

6358. The method of item 6356 wherein the agent is an AKT inhibitor.

6359. The method of item 6356 wherein the agent is an alpha 2 integrin antagonist, wherein the alpha 2 integrin antagonist is Pharmaprojects No. 5754 (Merck KgaA).

6360. The method of item 6356 wherein the agent is an alpha 4 integrin antagonist.

6361. The method of item 6356 wherein the agent is an alpha 7 nicotinic receptor agonist.

6362. The method of item 6356 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), §D-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

6363. The method of item 6356 wherein the agent is an apoptosis antagonist.

6364. The method of item 6356 wherein the agent is an apoptosis activator.

6365. The method of item 6356 wherein the agent is a beta 1 integrin antagonist.

6366. The method of item 6356 wherein the agent is a beta tubulin inhibitor.

6367. The method of item 6356 wherein the agent is a blocker of enzyme production in Hepatitis C.

6368. The method of item 6356 wherein the agent is a Bruton's tyrosine kinase inhibitor.

6369. The method of item 6356 wherein the agent is a calcineurin inhibitor.

6370. The method of item 6356 wherein the agent is a caspase 3 inhibitor.

6371. The method of item 6356 wherein the agent is a CC chemokine receptor antagonist.

6372. The method of item 6356 wherein the agent is a cell cycle inhibitor selected from the group consisting of SNS-595 (Sunesis), synthadotin, KRX-0403, and an analogue or derivative thereof.

6373. The method of item 6356 wherein the agent is a cathepsin B inhibitor.

6374. The method of item 6356 wherein the agent is a cathepsin K inhibitor, wherein the cathepsin K inhibitor is 462795 (GlaxoSmithKline), INPL-022-D6 (Amura Therapeutics), or an analogue or derivative thereof.

6375. The method of item 6356 wherein the agent is a cathepsin L inhibitor.

6376. The method of item 6356 wherein the agent is a CD40 antagonist.

6377. The method of item 6356 wherein the agent is a chemokine receptor agonist.

6378. The method of item 6356 wherein the agent is a chymase inhibitor.

6379. The method of item 6356 wherein the agent is a collagenase antagonist.

6380. The method of item 6356 wherein the agent is a CXCR antagonist.

6381. The method of item 6356 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

6382. The method of item 6356 wherein the agent is a cyclooxygenase 1 inhibitor.

6383. The method of item 6356 wherein the agent is a DHFR inhibitor.

6384. The method of item 6356 wherein the agent is a dual integrin inhibitor.

6385. The method of item 6356 wherein the agent is an elastase inhibitor.

6386. The method of item 6356 wherein the agent is an elongation factor-1 alpha inhibitor.

6387. The method of item 6356 wherein the agent is an endothelial growth factor antagonist.

6388. The method of item 6356 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

6389. The method of item 6356 wherein the agent is an endotoxin antagonist.

6390. The method of item 6356 wherein the agent is an epothilone and tubulin binder.

6391. The method of item 6356 wherein the agent is an estrogen receptor antagonist.

6392. The method of item 6356 wherein the agent is an FGF inhibitor.

6393. The method of item 6356 wherein the agent is a farnexyl transferase inhibitor.

6394. The method of item 6356 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

6395. The method of item 6356 wherein the agent is an FLT-3 kinase inhibitor.

6396. The method of item 6356 wherein the agent is an FGF receptor kinase inhibitor.

6397. The method of item 6356 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

6398. The method of item 6356 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

6399. The method of item 6356 wherein the agent is a histone deacetylase inhibitor.

6400. The method of item 6356 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

6401. The method of item 6356 wherein the agent is an ICAM inhibitor.

6402. The method of item 6356 wherein the agent is an IL, ICE and IRAK antagonist, wherein the antagonist is a CJ-14877, CP-424174 (Pfizer), NF-61 (Negma-Lerads), and an analogue or derivative thereof.

6403. The method of item 6356 wherein the agent is an IL-2 inhibitor.

6404. The method of item 6356 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

6405. The method of item 6356 wherein the agent is an IMPDH (inosine monophosphate).

6406. The method of item 6356 wherein the agent is an integrin antagonist.

6407. The method of item 6356 wherein the agent is an interleukin antagonist.

6408. The method of item 6356 wherein the agent is an inhibitor of type III receptor tyrosine kinase.

6409. The method of item 6356 wherein the agent is an irreversible inhibitor of enzyme methionine aminopeptidase type 2.

6410. The method of item 6356 wherein the agent is an isozyme selective delta protein kinase C inhibitor.

6411. The method of item 6356 wherein the agent a JAK3 enzyme inhibitor.

6412. The method of item 6356 wherein the agent is a JNK inhibitor.

6413. The method of item 6356 wherein the agent is a kinase inhibitor.

6414. The method of item 6356 wherein the agent is kinesin antagonist.

6415. The method of item 6356 wherein the agent is a kinesin antagonist.

6416. The method of item 6356 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

6417. The method of item 6356 wherein the agent is an MAP kinase inhibitor.

6418. The method of item 6356 wherein the agent is a matrix metalloproteinase inhibitor.

6419. The method of item 6356 wherein the agent is an MCP-CCR2 inhibitor.

6420. The method of item 6356 wherein the agent is an mTOR inhibitor.

6421. The method of item 6356 wherein the agent is an mTOR kinase inhibitor.

6422. The method of item 6356 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

6423. The method of item 6356 wherein the agent is an MIF inhibitor.

6424. The method of item 6356 wherein the agent is an MMP inhibitor.

6425. The method of item 6356 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

6426. The method of item 6356 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

6427. The method of item 6356 wherein the agent is a nitric oxide agonist.

6428. The method of item 6356 wherein the agent is an ornithine decarboxylase inhibitor.

6429. The method of item 6356 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

6430. The method of item 6356 wherein the agent is a palmitoyl-protein thioesterase inhibitor.

6431. The method of item 6356 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

6432. The method of item 6356 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

6433. The method of item 6356 wherein the agent is a phosphatase inhibitor.

6434. The method of item 6356 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

6435. The method of item 6356 wherein the agent is a PKC inhibitor.

6436. The method of item 6356 wherein the agent is a platelet activating factor antagonist.

6437. The method of item 6356 wherein the agent is a platelet-derived growth factor receptor kinase inhibitor.

6438. The method of item 6356 wherein the agent is a prolyl hydroxylase inhibitor.

6439. The method of item 6356 wherein the agent is a polymorphonuclear neutrophil inhibitor.

6440. The method of item 6356 wherein the agent is a protein kinase B inhibitor.

6441. The method of item 6356 wherein the agent is a protein kinase C stimulant.

6442. The method of item 6356 wherein the agent is a purine nucleoside analogue.

6443. The method of item 6356 wherein the agent is a purinoreceptor P2X antagonist.

6444. The method of item 6356 wherein the agent is a Raf kinase inhibitor.

6445. The method of item 6356 wherein the agent is a reversible inhibitor of ErbB1 and ErbB2.

6446. The method of item 6356 wherein the agent is a ribonucleoside triphosphate reductase inhibitor.

6447. The method of item 6356 wherein the agent is an SDF-1 antagonist.

6448. The method of item 6356 wherein the agent is a sheddase inhibitor.

6449. The method of item 6356 wherein the agent is an SRC inhibitor.

6450. The method of item 6356 wherein the agent is a stromelysin inhibitor.

6451. The method of item 6356 wherein the agent is an Syk kinase inhibitor.

6452. The method of item 6356 wherein the agent is a telomerase inhibitor.

6453. The method of item 6356 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

6454. The method of item 6356 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

6455. The method of item 6356 wherein the agent is a Toll receptor inhibitor.

6456. The method of item 6356 wherein the agent is a tubulin antagonist.

6457. The method of item 6356 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

6458. The method of item 6356 wherein the agent is a VEGF inhibitor.

6459. The method of item 6356 wherein the agent is a vitamin D receptor agonist.

6460. The method of item 6356 wherein the agent is ZD-6474 (an angiogenesis inhibitor).

6461. The method of item 6356 wherein the agent is AP-23573 (an mTOR inhibitor).

6462. The method of item 6356 wherein the agent is synthadotin (a tubulin antagonist).

6463. The method of item 6356 wherein the agent is S-0885 (a collagenase inhibitor).

6464. The method of item 6356 wherein the agent is aplidine (an elongation factor-1 alpha inhibitor).

6465. The method of item 6356 wherein the agent is ixabepilone (an epithilone).

6466. The method of item 6356 wherein the agent is IDN-5390 (an angiogenesis inhibitor).

6467. The method of item 6356 wherein the agent is SB-2723005 (an angiogenesis inhibitor).

6468. The method of item 6356 wherein the agent is ABT-518 (an angiogenesis inhibitor).

6469. The method of item 6356 wherein the agent is combretastatin (an angiogenesis inhibitor).

6470. The method of item 6356 wherein the agent is anecortave acetate (an angiogenesis inhibitor).

6471. The method of item 6356 wherein the agent is SB-715992 (a kinesin antagonist).

6472. The method of item 6356 wherein the agent is temsirolimus (an mTOR inhibitor).

6473. The method of item 6356 wherein the agent is adalimumab (a TNFα antagonist).

6474. The method of item 6356, wherein the composition comprises a polymer.

6475. The method of item 6356, wherein the composition comprises a polymer, and the polymer is, or comprises, a copolymer.

6476. The method of item 6356, wherein the composition comprises a polymer, and the polymer is, or comprises, a block copolymer.

6477. The method of item 6356, wherein the composition comprises a polymer, and the polymer is, or comprises, a random copolymer.

6478. The method of item 6356, wherein the composition comprises a polymer, and the polymer is, or comprises, a biodegradable polymer.

6479. The method of item 6356, wherein the composition comprises a polymer, and the polymer is, or comprises, a non-biodegradable polymer.

6480. The method of item 6356, wherein the composition comprises a polymer, and the polymer is, or comprises, a hydrophilic polymer.

6481. The method of item 6356, wherein the composition comprises a polymer, and the polymer is, or comprises, a hydrophobic polymer.

6482. The method of item 6356, wherein the composition comprises a polymer, and the polymer is, or comprises, a polymer having hydrophilic domains.

6483. The method of item 6356, wherein the composition comprises a polymer, and the polymer is, or comprises, a polymer having hydrophobic domains.

6484. The method of item 6356, wherein the composition comprises a polymer, and the polymer is, or comprises, a non-conductive polymer.

6485. The method of item 6356, wherein the composition comprises a polymer, and the polymer is, or comprises, an elastomer.

6486. The method of item 6356, wherein the composition comprises a polymer, and the polymer is, or comprises, a hydrogel.

6487. The method of item 6356, wherein the composition comprises a polymer, and the polymer is, or comprises, a silicone polymer.

6488. The method of item 6356, wherein the composition comprises a polymer, and the polymer is, or comprises, a hydrocarbon polymer.

6489. The method of item 6356, wherein the composition comprises a polymer, and the polymer is, or comprises, a styrene-derived polymer.

6490. The method of item 6356, wherein the composition comprises a polymer, and the polymer is, or comprises, a butadiene-derived polymer.

6491. The method of item 6356, wherein the composition comprises a polymer, and the polymer is, or comprises, a macromer.

6492. The method of item 6356, wherein the composition comprises a polymer, and the polymer is, or comprises, a poly(ethylene glycol) polymer.

6493. The method of item 6356, wherein the composition comprises a polymer, and the polymer is, or comprises, an amorphous polymer.

6494. The method of item 6356, wherein the composition further comprises a second pharmaceutically active agent.

6495. The method of item 6356, wherein the composition further comprises an anti-inflammatory agent.

6496. The method of item 6356, wherein the composition further comprises an agent that inhibits infection.

6497. The method of item 6356, wherein the composition further comprises an anthracycline.

6498. The method of item 6356, wherein the composition further comprises doxorubicin.

6499. The method of item 6356 wherein the composition further comprises mitoxantrone.

6500. The method of item 6356 wherein the composition further comprises a fluoropyrimidine.

6501. The method of item 6356, wherein the composition further comprises 5-fluorouracil (5-FU).

6502. The method of item 6356, wherein the composition further comprises a folic acid antagonist.

6503. The method of item 6356, wherein the composition further comprises methotrexate.

6504. The method of item 6356, wherein the composition further comprises a podophylotoxin.

6505. The method of item 6356, wherein the composition further comprises etoposide.

6506. The method of item 6356, wherein the composition further comprises camptothecin.

6507. The method of item 6356, wherein the composition further comprises a hydroxyurea.

6508. The method of item 6356, wherein the composition further comprises a platinum complex.

6509. The method of item 6356, wherein the composition further comprises cisplatin.

6510. The method of item 6356 wherein the composition further comprises an anti-thrombotic agent.

6511. The method of item 6356, wherein the composition further comprises a visualization agent.

6512. The method of item 6356, wherein the composition further comprises a visualization agent, and the visualization agent is a radiopaque material, wherein the radiopaque material comprises a metal, a halogenated compound, or a barium containing compound.

6513. The method of item 6356, wherein the composition further comprises a visualization agent, and the visualization agent is, or comprises, barium, tantalum, or technetium.

6514. The method of item 6356, wherein the composition further comprises a visualization agent, and the visualization agent is, or comprises, an MRI responsive material.

6515. The method of item 6356, wherein the composition further comprises a visualization agent, and the visualization agent is, or comprises, a gadolinium chelate.

6516. The method of item 6356, wherein the composition further comprises a visualization agent, and the visualization agent is, or comprises, iron, magnesium, manganese, copper, or chromium.

6517. The method of item 6356, wherein the composition further comprises a visualization agent, and the visualization agent is, or comprises, iron oxide compound.

6518. The method of item 6356, wherein the composition further comprises a visualization agent, and the visualization agent is, or comprises, a dye, pigment, or colorant.

6519. The method of item 6356 wherein the agent is released in effective concentrations from the composition comprising the agent by diffusion over a period ranging from the time of administration to about 90 days.

6520. The method of item 6356 wherein the agent is released in effective concentrations from the composition comprising the agent by erosion of the composition over a period ranging from the time of administration to about 90 days.

6521. The method of item 6356 wherein the composition further comprises an inflammatory cytokine.

6522. The method of item 6356 wherein the composition further comprises an agent that stimulates cell proliferation.

6523. The method of item 6356 wherein the composition further comprises a polymeric carrier.

6524. The method of item 6356 wherein the composition is in the form of a gel, paste, or spray.

6525. The method of item 6356 wherein the electrical device is partially constructed with the agent or the composition.

6526. The method of item 6356 wherein the electrical device is impregnated with the agent or the composition.

6527. The method of item 6356, wherein the agent or the composition forms a coating, and the coating directly contacts the electrical device.

6528. The method of item 6356, wherein the agent or the composition forms a coating, and the coating indirectly contacts the electrical device.

6529. The method of item 6356 wherein the agent or the composition forms a coating, and the coating partially covers the electrical device.

6530. The method of item 6356, wherein the agent or the composition forms a coating, and the coating completely covers the electrical device.

6531. The method of item 6356 wherein the agent or the composition is located within pores or holes of the electrical device.

6532. The method of item 6356 wherein the agent or the composition is located within a channel, lumen, or divet of the electrical device.

6533. The method of item 6356 wherein the electrical device further comprises an echogenic material.

6534. The method of item 6356 wherein the electrical device further comprises an echogenic material, wherein the echogenic material is in the form of a coating.

6535. The method of item 6356 wherein the electrical device is sterile.

6536. The method of item 6356 wherein the agent is delivered from the electrical device, wherein the agent is released into tissue in the vicinity of the electrical device after deployment of the electrical device.

6537. The method of item 6356 wherein the agent is delivered from the electrical device, wherein the agent is released into tissue in the vicinity of the electrical device after deployment of the electrical device, wherein the tissue is connective tissue.

6538. The method of item 6356 wherein the agent is delivered from the electrical device, wherein the agent is released into tissue in the vicinity of the electrical device after deployment of the electrical device, wherein the tissue is muscle tissue.

6539. The method of item 6356 wherein the agent is delivered from the electrical device, wherein the agent is released into tissue in the vicinity of the electrical device after deployment of the electrical device, wherein the tissue is nerve tissue.

6540. The method of item 6356 wherein the agent is delivered from the electrical device, wherein the agent is released into tissue in the vicinity of the electrical device after deployment of the electrical device, wherein the tissue is epithelium tissue.

6541. The method of item 6356 wherein the agent is delivered from the electrical device, wherein the agent is released in effective concentrations from the electrical device over a period ranging from the time of deployment of the electrical device to about 1 year.

6542. The method of item 6356 wherein the agent is delivered from the electrical device, wherein the agent is released in effective concentrations from the electrical device over a period ranging from about 1 month to 6 months.

6543. The method of item 6356 wherein the agent is delivered from the electrical device, wherein the agent is released in effective concentrations from the electrical device over a period ranging from about 1-90 days.

6544. The method of item 6356 wherein the agent is delivered from the electrical device, wherein the agent is released in effective concentrations from the electrical device at a constant rate.

6545. The method of item 6356 wherein the agent is delivered from the electrical device, wherein the agent is released in effective concentrations from the electrical device at an increasing rate.

6546. The method of item 6356 wherein the agent is delivered from the electrical device, wherein the agent is released in effective concentrations from the electrical device at a decreasing rate.

6547. The method of item 6356 wherein the agent is delivered from the electrical device, wherein the electrical device comprises about 0.01 μg to about 10 μg of the agent.

6548. The method of item 6356 wherein the agent is delivered from the electrical device, wherein the electrical device comprises about 10 μg to about 10 mg of the agent.

6549. The method of item 6356 wherein the agent is delivered from the electrical device, wherein the electrical device comprises about 10 mg to about 250 mg of the agent.

6550. The method of item 6356 wherein the agent is delivered from the electrical device, wherein the electrical device comprises about 250 mg to about 1000 mg of the agent.

6551. The method of item 6356 wherein the agent is delivered from the electrical device, wherein the electrical device comprises about 1000 mg to about 2500 mg of the agent.

6552. The method of item 6356 wherein the agent is delivered from the electrical device, wherein a surface of the electrical device comprises less than 0.01 μg of the agent per mm² of electrical device surface to which the agent is applied.

6553. The method of item 6356 wherein the agent is delivered from the electrical device, wherein a surface of the electrical device comprises about 0.01 μg to about 1 μg of the agent per mm² of electrical device surface to which the agent is applied.

6554. The method of item 6356 wherein the agent is delivered from the electrical device, wherein a surface of the electrical device comprises about 1 μg to about 10 μg of the agent per mm² of electrical device surface to which the agent is applied.

6555. The method of item 6356 wherein the agent is delivered from the electrical device, wherein a surface of the electrical device comprises about 10 μg to about 250 μg of the agent per mm² of electrical device surface to which the agent is applied.

6556. The method of item 6356 wherein the agent is delivered from the electrical device, wherein a surface of the electrical device comprises about 250 μg to about 1000 μg of the agent per mm² of electrical device surface to which the agent is applied.

6557. The method of item 6356 wherein the agent is delivered from the electrical device, wherein a surface of the electrical device comprises about 1000 μg to about 2500 μg of the agent per mm² of electrical device surface to which the agent is applied.

6558. The method of item 6356, wherein the electrical device further comprises a coating, and the coating is a uniform coating.

6559. The method of item 6356, wherein the electrical device further comprises a coating, and the coating is a non-uniform coating.

6560. The method of item 6356, wherein the electrical device further comprises a coating, and the coating is a discontinuous coating.

6561. The method of item 6356, wherein the electrical device further comprises a coating, and the coating is a patterned coating.

6562. The method of item 6356, wherein the electrical device further comprises a coating, and the coating has a thickness of 100 μm or less.

6563. The method of item 6356, wherein the electrical device further comprises a coating, and the coating has a thickness of 10 μm or less.

6564. The method of item 6356, wherein the electrical device further comprises a coating, and the coating adheres to the surface of the electrical device upon deployment of the electrical device

6565. The method of item 6356, wherein the electrical device further comprises a coating, and the coating is stable at room temperature for a period of at least 1 year.

6566. The method of item 6356, wherein the electrical device further comprises a coating, and the agent is present in the coating in an amount ranging between about 0.0001% to about 1% by weight.

6567. The method of item 6356, wherein the electrical device further comprises a coating, and the agent is present in the coating in an amount ranging between about 1% to about 10% by weight.

6568. The method of item 6356, wherein the electrical device further comprises a coating, and the agent is present in the coating in an amount ranging between about 10% to about 25% by weight.

6569. The method of item 6356, wherein the electrical device further comprises a coating, and the agent is present in the coating in an amount ranging between about 25% to about 70% by weight.

6570. The method of item 6356, wherein the electrical device further comprises a coating, and the coating comprises a polymer.

6571. The method of item 6356, wherein the electrical device comprises a first coating having a first composition and a second coating having a second composition.

6572. The method of item 6356, wherein the electrical device comprises a first coating having a first composition and a second coating having a second composition, wherein the first composition and the second composition are different.

6573. The method of item 6356 wherein the agent or the composition is affixed to the electrical device.

6574. The method of item 6356 wherein the agent or the composition is covalently attached to the electrical device.

6575. The method of item 6356 wherein the agent or the composition is non-covalently attached to the electrical device.

6576. The method of item 6356 wherein the electrical device comprises a coating that absorbs the agent or the composition.

6577. The method of item 6356 wherein the electrical device is interweaved with a thread composed of, or coated with, the agent or the composition.

6578. The method of item 6356 wherein a portion of the electrical device is covered with a sleeve that contains the agent or the composition.

6579. The method of item 6356 wherein the electrical device is completely covered with a sleeve that contains the agent or the composition.

6580. The method of item 6356 wherein a portion of the electrical device is covered with a mesh that contains the agent or the composition.

6581. The method of item 6356 wherein the electrical device is completely covered with a mesh that contains the agent or the composition.

6582. The method of item 6356 wherein the agent or the composition is applied to the electrical device surface prior to the placing of the electrical device into the host.

6583. The method of item 6356 wherein the agent or the composition is applied to the electrical device surface during the placing of the electrical device into the host.

6584. The method of item 6356 wherein the agent or the composition is applied to the electrical device surface immediately after the placing of the electrical device into the host.

6585. The method of item 6356 wherein the agent or the composition is applied to the surface of the tissue in the host surrounding the electrical device prior to the placing of the electrical device into the host.

6586. The method of item 6356 wherein the agent or the composition is applied to the surface of the tissue in the host surrounding the electrical device during the placing of the electrical device into the host.

6587. The method of item 6356 wherein the agent or the composition is applied to the surface of the tissue in the host surrounding the electrical device immediately after the placing of the electrical device into the host.

6588. The method of item 6356 wherein the agent or the composition is topically applied into the anatomical space where the electrical device is placed.

6589. The method of item 6356 wherein the agent or the composition is percutaneously injected into the tissue in the host surrounding the electrical device.

6590. The method for inhibiting scarring of any one of items 6356-6589 wherein the vagus nerve stimulator is adapted for treating supraventricular arrhythmias.

6591. The method for inhibiting scarring of any one of items 6356-6589 wherein the vagus nerve stimulator is adapted for treating angina pectoris.

6592. The method for inhibiting scarring of any one of items 6356-6589 wherein the vagus nerve stimulator is adapted for treating atrial tachycardia.

6593. The method for inhibiting scarring of any one of items 6356-6589 wherein the vagus nerve stimulator is adapted for treating atrial flutter.

6594. The method for inhibiting scarring of any one of items 6356-6589 wherein the vagus nerve stimulator is adapted for treating arterial fibrillation.

6595. The method for inhibiting scarring of any one of items 6356-6589 wherein the vagus nerve stimulator is arrhythmias that result in low cardiac output.

6596. The method for inhibiting scarring of any one of items 6356-6589 wherein the vagus nerve stimulator comprises a programmable pulse generator.

6597. A method for inhibiting scarring comprising placing an electrical lead (i.e., an electrical device) and an anti-scarring agent or a composition comprising an ant-scarring agent into an animal host, wherein the agent inhibits scarring.

6598. The method of item 6597 wherein the agent is an adensosine A2A receptor antagonist.

6599. The method of item 6597 wherein the agent is an AKT inhibitor.

6600. The method of item 6597 wherein the agent is an alpha 2 integrin antagonist, wherein the alpha 2 integrin antagonist is Pharmaprojects No. 5754 (Merck KgaA).

6601. The method of item 6597 wherein the agent is an alpha 4 integrin antagonist.

6602. The method of item 6597 wherein the agent is an alpha 7 nicotinic receptor agonist.

6603. The method of item 6597 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

6604. The method of item 6597 wherein the agent is an apoptosis antagonist.

6605. The method of item 6597 wherein the agent is an apoptosis activator.

6606. The method of item 6597 wherein the agent is a beta 1 integrin antagonist.

6607. The method of item 6597 wherein the agent is a beta tubulin inhibitor.

6608. The method of item 6597 wherein the agent is a blocker of enzyme production in Hepatitis C.

6609. The method of item 6597 wherein the agent is a Bruton's tyrosine kinase inhibitor.

6610. The method of item 6597 wherein the agent is a calcineurin inhibitor.

6611. The method of item 6597 wherein the agent is a caspase 3 inhibitor.

6612. The method of item 6597 wherein the agent is a CC chemokine receptor antagonist.

6613. The method of item 6597 wherein the agent is a cell cycle inhibitor selected from the group consisting of SNS-595 (Sunesis), synthadotin, KRX-0403, and an analogue or derivative thereof.

6614. The method of item 6597 wherein the agent is a cathepsin B inhibitor.

6615. The method of item 6597 wherein the agent is a cathepsin K inhibitor, wherein the cathepsin K inhibitor is 462795 (GlaxoSmithKline), INPL-022-D6 (Amura Therapeutics), or an analogue or derivative thereof.

6616. The method of item 6597 wherein the agent is a cathepsin L inhibitor.

6617. The method of item 6597 wherein the agent is a CD40 antagonist.

6618. The method of item 6597 wherein the agent is a chemokine receptor agonist.

6619. The method of item 6597 wherein the agent is a chymase inhibitor.

6620. The method of item 6597 wherein the agent is a collagenase antagonist.

6621. The method of item 6597 wherein the agent is a CXCR antagonist.

6622. The method of item 6597 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

6623. The method of item 6597 wherein the agent is a cyclooxygenase 1 inhibitor.

6624. The method of item 6597 wherein the agent is a DHFR inhibitor.

6625. The method of item 6597 wherein the agent is a dual integrin inhibitor.

6626. The method of item 6597 wherein the agent is an elastase inhibitor.

6627. The method of item 6597 wherein the agent is an elongation factor-1 alpha inhibitor.

6628. The method of item 6597 wherein the agent is an endothelial growth factor antagonist.

6629. The method of item 6597 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

6630. The method of item 6597 wherein the agent is an endotoxin antagonist.

6631. The method of item 6597 wherein the agent is an epothilone and tubulin binder.

6632. The method of item 6597 wherein the agent is an estrogen receptor antagonist.

6633. The method of item 6597 wherein the agent is an FGF inhibitor.

6634. The method of item 6597 wherein the agent is a farnexyl transferase inhibitor.

6635. The method of item 6597 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

6636. The method of item 6597 wherein the agent is an FLT-3 kinase inhibitor.

6637. The method of item 6597 wherein the agent is an FGF receptor kinase inhibitor.

6638. The method of item 6597 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

6639. The method of item 6597 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

6640. The method of item 6597 wherein the agent is a histone deacetylase inhibitor.

6641. The method of item 6597 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

6642. The method of item 6597 wherein the agent is an ICAM inhibitor.

6643. The method of item 6597 wherein the agent is an IL, ICE and IRAK antagonist, wherein the antagonist is a CJ-14877, CP-424174 (Pfizer), NF-61 (Negma-Lerads), and an analogue or derivative thereof.

6644. The method of item 6597 wherein the agent is an IL-2 inhibitor.

6645. The method of item 6597 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

6646. The method of item 6597 wherein the agent is an IMPDH (inosine monophosphate).

6647. The method of item 6597 wherein the agent is an integrin antagonist.

6648. The method of item 6597 wherein the agent is an interleukin antagonist.

6649. The method of item 6597 wherein the agent is an inhibitor of type III receptor tyrosine kinase.

6650. The method of item 6597 wherein the agent is an irreversible inhibitor of enzyme methionine aminopeptidase type 2.

6651. The method of item 6597 wherein the agent is an isozyme selective delta protein kinase C inhibitor.

6652. The method of item 6597 wherein the agent a JAK3 enzyme inhibitor.

6653. The method of item 6597 wherein the agent is a JNK inhibitor.

6654. The method of item 6597 wherein the agent is a kinase inhibitor.

6655. The method of item 6597 wherein the agent is kinesin antagonist.

6656. The method of item 6597 wherein the agent is a kinesin antagonist.

6657. The method of item 6597 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

6658. The method of item 6597 wherein the agent is an MAP kinase inhibitor.

6659. The method of item 6597 wherein the agent is a matrix metalloproteinase inhibitor.

6660. The method of item 6597 wherein the agent is an MCP-CCR2 inhibitor.

6661. The method of item 6597 wherein the agent is an mTOR inhibitor.

6662. The method of item 6597 wherein the agent is an mTOR kinase inhibitor.

6663. The method of item 6597 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

6664. The method of item 6597 wherein the agent is an MIF inhibitor.

6665. The method of item 6597 wherein the agent is an MMP inhibitor.

6666. The method of item 6597 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

6667. The method of item 6597 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

6668. The method of item 6597 wherein the agent is a nitric oxide agonist.

6669. The method of item 6597 wherein the agent is an ornithine decarboxylase inhibitor

6670. The method of item 6597 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

6671. The method of item 6597 wherein the agent is a palmitoyl-protein thioesterase inhibitor.

6672. The method of item 6597 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

6673. The method of item 6597 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

6674. The method of item 6597 wherein the agent is a phosphatase inhibitor.

6675. The method of item 6597 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

6676. The method of item 6597 wherein the agent is a PKC inhibitor.

6677. The method of item 6597 wherein the agent is a platelet activating factor antagonist.

6678. The method of item 6597 wherein the agent is a platelet-derived growth factor receptor kinase inhibitor.

6679. The method of item 6597 wherein the agent is a prolyl hydroxylase inhibitor.

6680. The method of item 6597 wherein the agent is a polymorphonuclear neutrophil inhibitor.

6681. The method of item 6597 wherein the agent is a protein kinase B inhibitor.

6682. The method of item 6597 wherein the agent is a protein kinase C stimulant.

6683. The method of item 6597 wherein the agent is a purine nucleoside analogue.

6684. The method of item 6597 wherein the agent is a purinoreceptor P2X antagonist.

6685. The method of item 6597 wherein the agent is a Raf kinase inhibitor.

6686. The method of item 6597 wherein the agent is a reversible inhibitor of ErbB1 and ErbB2.

6687. The method of item 6597 wherein the agent is a ribonucleoside triphosphate reductase inhibitor.

6688. The method of item 6597 wherein the agent is an SDF-1 antagonist.

6689. The method of item 6597 wherein the agent is a sheddase inhibitor.

6690. The method of item 6597 wherein the agent is an SRC inhibitor.

6691. The method of item 6597 wherein the agent is a stromelysin inhibitor.

6692. The method of item 6597 wherein the agent is an Syk kinase inhibitor.

6693. The method of item 6597 wherein the agent is a telomerase inhibitor.

6694. The method of item 6597 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

6695. The method of item 6597 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

6696. The method of item 6597 wherein the agent is a Toll receptor inhibitor.

6697. The method of item 6597 wherein the agent is a tubulin antagonist.

6698. The method of item 6597 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 14920442-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

6699. The method of item 6597 wherein the agent is a VEGF inhibitor.

6700. The method of item 6597 wherein the agent is a vitamin D receptor agonist.

6701. The method of item 6597 wherein the agent is ZD-6474 (an angiogenesis inhibitor).

6702. The method of item 6597 wherein the agent is AP-23573 (an mTOR inhibitor).

6703. The method of item 6597 wherein the agent is synthadotin (a tubulin antagonist).

6704. The method of item 6597 wherein the agent is S-0885 (a collagenase inhibitor).

6705. The method of item 6597 wherein the agent is aplidine (an elongation factor-1 alpha inhibitor).

6706. The method of item 6597 wherein the agent is ixabepilone (an epithilone).

6707. The method of item 6597 wherein the agent is IDN-5390 (an angiogenesis inhibitor).

6708. The method of item 6597 wherein the agent is SB-2723005 (an angiogenesis inhibitor).

6709. The method of item 6597 wherein the agent is ABT-518 (an angiogenesis inhibitor).

6710. The method of item 6597 wherein the agent is combretastatin (an angiogenesis inhibitor).

6711. The method of item 6597 wherein the agent is anecortave acetate (an angiogenesis inhibitor).

6712. The method of item 6597 wherein the agent is SB-715992 (a kinesin antagonist).

6713. The method of item 6597 wherein the agent is temsirolimus (an mTOR inhibitor).

6714. The method of item 6597 wherein the agent is adalimumab (a TNFα antagonist).

6715. The method of item 6597, wherein the composition comprises a polymer.

6716. The method of item 6597, wherein the composition comprises a polymer, and the polymer is, or comprises, a copolymer.

6717. The method of item 6597, wherein the composition comprises a polymer, and the polymer is, or comprises, a block copolymer.

6718. The method of item 6597, wherein the composition comprises a polymer, and the polymer is, or comprises, a random copolymer.

6719. The method of item 6597, wherein the composition comprises a polymer, and the polymer is, or comprises, a biodegradable polymer.

6720. The method of item 6597, wherein the composition comprises a polymer, and the polymer is, or comprises, a non-biodegradable polymer.

6721. The method of item 6597, wherein the composition comprises a polymer, and the polymer is, or comprises, a hydrophilic polymer.

6722. The method of item 6597, wherein the composition comprises a polymer, and the polymer is, or comprises, a hydrophobic polymer.

6723. The method of item 6597, wherein the composition comprises a polymer, and the polymer is, or comprises, a polymer having hydrophilic domains.

6724. The method of item 6597, wherein the composition comprises a polymer, and the polymer is, or comprises, a polymer having hydrophobic domains.

6725. The method of item 6597, wherein the composition comprises a polymer, and the polymer is, or comprises, a non-conductive polymer.

6726. The method of item 6597, wherein the composition comprises a polymer, and the polymer is, or comprises, an elastomer.

6727. The method of item 6597, wherein the composition comprises a polymer, and the polymer is, or comprises, a hydrogel.

6728. The method of item 6597, wherein the composition comprises a polymer, and the polymer is, or comprises, a silicone polymer.

6729. The method of item 6597, wherein the composition comprises a polymer, and the polymer is, or comprises, a hydrocarbon polymer.

6730. The method of item 6597, wherein the composition comprises a polymer, and the polymer is, or comprises, a styrene-derived polymer.

6731. The method of item 6597, wherein the composition comprises a polymer, and the polymer is, or comprises, a butadiene-derived polymer.

6732. The method of item 6597, wherein the composition comprises a polymer, and the polymer is, or comprises, a macromer.

6733. The method of item 6597, wherein the composition comprises a polymer, and the polymer is, or comprises, a poly(ethylene glycol) polymer.

6734. The method of item 6597, wherein the composition comprises a polymer, and the polymer is, or comprises, an amorphous polymer.

6735. The method of item 6597, wherein the composition further comprises a second pharmaceutically active agent.

6736. The method of item 6597, wherein the composition further comprises an anti-inflammatory agent.

6737. The method of item 6597, wherein the composition further comprises an agent that inhibits infection.

6738. The method of item 6597, wherein the composition further comprises an anthracycline.

6739. The method of item 6597, wherein the composition further comprises doxorubicin.

6740. The method of item 6597 wherein the composition further comprises mitoxantrone.

6741. The method of item 6597 wherein the composition further comprises a fluoropyrimidine.

6742. The method of item 6597, wherein the composition further comprises 5-fluorouracil (5-FU).

6743. The method of item 6597, wherein the composition further comprises a folic acid antagonist.

6744. The method of item 6597, wherein the composition further comprises methotrexate.

6745. The method of item 6597, wherein the composition further comprises a podophylotoxin.

6746. The method of item 6597, wherein the composition further comprises etoposide.

6747. The method of item 6597, wherein the composition further comprises camptothecin.

6748. The method of item 6597, wherein the composition further comprises a hydroxyurea.

6749. The method of item 6597, wherein the composition further comprises a platinum complex.

6750. The method of item 6597, wherein the composition further comprises cisplatin.

6751. The method of item 6597 wherein the composition further comprises an anti-thrombotic agent.

6752. The method of item 6597, wherein the composition further comprises a visualization agent.

6753. The method of item 6597, wherein the composition further comprises a visualization agent, and the visualization agent is a radiopaque material, wherein the radiopaque material comprises a metal, a halogenated compound, or a barium containing compound.

6754. The method of item 6597, wherein the composition further comprises a visualization agent, and the visualization agent is, or comprises, barium, tantalum, or technetium.

6755. The method of item 6597, wherein the composition further comprises a visualization agent, and the visualization agent is, or comprises, an MRI responsive material.

6756. The method of item 6597, wherein the composition further comprises a visualization agent, and the visualization agent is, or comprises, a gadolinium chelate.

6757. The method of item 6597, wherein the composition further comprises a visualization agent, and the visualization agent is, or comprises, iron, magnesium, manganese, copper, or chromium.

6758. The method of item 6597, wherein the composition further comprises a visualization agent, and the visualization agent is, or comprises, iron oxide compound.

6759. The method of item 6597, wherein the composition further comprises a visualization agent, and the visualization agent is, or comprises, a dye, pigment, or colorant.

6760. The method of item 6597 wherein the agent is released in effective concentrations from the composition comprising the agent by diffusion over a period ranging from the time of administration to about 90 days.

6761. The method of item 6597 wherein the agent is released in effective concentrations from the composition comprising the agent by erosion of the composition over a period ranging from the time of administration to about 90 days.

6762. The method of item 6597 wherein the composition further comprises an inflammatory cytokine.

6763. The method of item 6597 wherein the composition further comprises an agent that stimulates cell proliferation.

6764. The method of item 6597 wherein the composition further comprises a polymeric carrier.

6765. The method of item 6597 wherein the composition is in the form of a gel, paste, or spray.

6766. The method of item 6597 wherein the electrical device is partially constructed with the agent or the composition.

6767. The method of item 6597 wherein the electrical device is impregnated with the agent or the composition.

6768. The method of item 6597, wherein the agent or the composition forms a coating, and the coating directly contacts the electrical device.

6769. The method of item 6597, wherein the agent or the composition forms a coating, and the coating indirectly contacts the electrical device.

6770. The method of item 6597 wherein the agent or the composition forms a coating, and the coating partially covers the electrical device.

6771. The method of item 6597, wherein the agent or the composition forms a coating, and the coating completely covers the electrical device.

6772. The method of item 6597 wherein the agent or the composition is located within pores or holes of the electrical device.

6773. The method of item 6597 wherein the agent or the composition is located within a channel, lumen, or divet of the electrical device.

6774. The method of item 6597 wherein the electrical device further comprises an echogenic material.

6775. The method of item 6597 wherein the electrical device further comprises an echogenic material, wherein the echogenic material is in the form of a coating.

6776. The method of item 6597 wherein the electrical device is sterile.

6777. The method of item 6597 wherein the agent is delivered from the electrical device, wherein the agent is released into tissue in the vicinity of the electrical device after deployment of the electrical device.

6778. The method of item 6597 wherein the agent is delivered from the electrical device, wherein the agent is released into tissue in the vicinity of the electrical device after deployment of the electrical device, wherein the tissue is connective tissue.

6779. The method of item 6597 wherein the agent is delivered from the electrical device, wherein the agent is released into tissue in the vicinity of the electrical device after deployment of the electrical device, wherein the tissue is muscle tissue

6780. The method of item 6597 wherein the agent is delivered from the electrical device, wherein the agent is released into tissue in the vicinity of the electrical device after deployment of the electrical device, wherein the tissue is nerve tissue.

6781. The method of item 6597 wherein the agent is delivered from the electrical device, wherein the agent is released into tissue in the vicinity of the electrical device after deployment of the electrical device, wherein the tissue is epithelium tissue.

6782. The method of item 6597 wherein the agent is delivered from the electrical device, wherein the agent is released in effective concentrations from the electrical device over a period ranging from the time of deployment of the electrical device to about 1 year.

6783. The method of item 6597 wherein the agent is delivered from the electrical device, wherein the agent is released in effective concentrations from the electrical device over a period ranging from about 1 month to 6 months.

6784. The method of item 6597 wherein the agent is delivered from the electrical device, wherein the agent is released in effective concentrations from the electrical device over a period ranging from about 1-90 days.

6785. The method of item 6597 wherein the agent is delivered from the electrical device, wherein the agent is released in effective concentrations from the electrical device at a constant rate.

6786. The method of item 6597 wherein the agent is delivered from the electrical device, wherein the agent is released in effective concentrations from the electrical device at an increasing rate.

6787. The method of item 6597 wherein the agent is delivered from the electrical device, wherein the agent is released in effective concentrations from the electrical device at a decreasing rate.

6788. The method of item 6597 wherein the agent is delivered from the electrical device, wherein the electrical device comprises about 0.01 μg to about 10 μg of the agent.

6789. The method of item 6597 wherein the agent is delivered from the electrical device, wherein the electrical device comprises about 10 μg to about 10 mg of the agent.

6790. The method of item 6597 wherein the agent is delivered from the electrical device, wherein the electrical device comprises about 10 mg to about 250 mg of the agent.

6791. The method of item 6597 wherein the agent is delivered from the electrical device, wherein the electrical device comprises about 250 mg to about 1000 mg of the agent.

6792. The method of item 6597 wherein the agent is delivered from the electrical device, wherein the electrical device comprises about 1000 mg to about 2500 mg of the agent.

6793. The method of item 6597 wherein the agent is delivered from the electrical device, wherein a surface of the electrical device comprises less than 0.01 μg of the agent per mm² of electrical device surface to which the agent is applied.

6794. The method of item 6597 wherein the agent is delivered from the electrical device, wherein a surface of the electrical device comprises about 0.01 μg to about 1 μg of the agent per mm² of electrical device surface to which the agent is applied.

6795. The method of item 6597 wherein the agent is delivered from the electrical device, wherein a surface of the electrical device comprises about 1 μg to about 10 μg of the agent per mm² of electrical device surface to which the agent is applied.

6796. The method of item 6597 wherein the agent is delivered from the electrical device, wherein a surface of the electrical device comprises about 10 μg to about 250 μg of the agent per mm² of electrical device surface to which the agent is applied.

6797. The method of item 6597 wherein the agent is delivered from the electrical device, wherein a surface of the electrical device comprises about 250 μg to about 1000 μg of the agent per mm² of electrical device surface to which the agent is applied.

6798. The method of item 6597 wherein the agent is delivered from the electrical device, wherein a surface of the electrical device comprises about 1000 μg to about 2500 μg of the agent per mm² of electrical device surface to which the agent is applied.

6799. The method of item 6597, wherein the electrical device further comprises a coating, and the coating is a uniform coating.

6800. The method of item 6597, wherein the electrical device further comprises a coating, and the coating is a non-uniform coating.

6801. The method of item 6597, wherein the electrical device further comprises a coating, and the coating is a discontinuous coating.

6802. The method of item 6597, wherein the electrical device further comprises a coating, and the coating is a patterned coating.

6803. The method of item 6597, wherein the electrical device further comprises a coating, and the coating has a thickness of 100 μm or less.

6804. The method of item 6597, wherein the electrical device further comprises a coating, and the coating has a thickness of 10 μm or less.

6805. The method of item 6597, wherein the electrical device further comprises a coating, and the coating adheres to the surface of the electrical device upon deployment of the electrical device.

6806. The method of item 6597, wherein the electrical device further comprises a coating, and the coating is stable at room temperature for a period of at least 1 year.

6807. The method of item 6597, wherein the electrical device further comprises a coating, and the agent is present in the coating in an amount ranging between about 0.0001% to about 1% by weight.

6808. The method of item 6597, wherein the electrical device further comprises a coating, and the agent is present in the coating in an amount ranging between about 1% to about 10% by weight.

6809. The method of item 6597, wherein the electrical device further comprises a coating, and the agent is present in the coating in an amount ranging between about 10% to about 25% by weight.

6810. The method of item 6597, wherein the electrical device further comprises a coating, and the agent is present in the coating in an amount ranging between about 25% to about 70% by weight.

6811. The method of item 6597, wherein the electrical device further comprises a coating, and the coating comprises a polymer.

6812. The method of item 6597, wherein the electrical device comprises a first coating having a first composition and a second coating having a second composition.

6813. The method of item 6597, wherein the electrical device comprises a first coating having a first composition and a second coating having a second composition, wherein the first composition and the second composition are different.

6814. The method of item 6597 wherein the agent or the composition is affixed to the electrical device.

6815. The method of item 6597 wherein the agent or the composition is covalently attached to the electrical device.

6816. The method of item 6597 wherein the agent or the composition is non-covalently attached to the electrical device.

6817. The method of item 6597 wherein the electrical device comprises a coating that absorbs the agent or the composition.

6818. The method of item 6597 wherein the electrical device is interweaved with a thread composed of, or coated with, the agent or the composition.

6819. The method of item 6597 wherein a portion of the electrical device is covered with a sleeve that contains the agent or the composition.

6820. The method of item 6597 wherein the electrical device is completely covered with a sleeve that contains the agent or the composition.

6821. The method of item 6597 wherein a portion of the electrical device is covered with a mesh that contains the agent or the composition.

6822. The method of item 6597 wherein the electrical device is completely covered with a mesh that contains the agent or the composition.

6823. The method of item 6597 wherein the agent or the composition is applied to the electrical device surface prior to the placing of the electrical device into the host

6824. The method of item 6597 wherein the agent or the composition is applied to the electrical device surface during the placing of the electrical device into the host.

6825. The method of item 6597 wherein the agent or the composition is applied to the electrical device surface immediately after the placing of the electrical device into the host.

6826. The method of item 6597 wherein the agent or the composition is applied to the surface of the tissue in the host surrounding the electrical device prior to the placing of the electrical device into the host.

6827. The method of item 6597 wherein the agent or the composition is applied to the surface of the tissue in the host surrounding the electrical device during the placing of the electrical device into the host.

6828. The method of item 6597 wherein the agent or the composition is applied to the surface of the tissue in the host surrounding the electrical device immediately after the placing of the electrical device into the host.

6829. The method of item 6597 wherein the agent or the composition is topically applied into the anatomical space where the electrical device is placed.

6830. The method of item 6597 wherein the agent or the composition is percutaneously injected into the tissue in the host surrounding the electrical device.

6831. The method for inhibiting scarring of any one of items 6597-6830 wherein the electrical lead comprises a connector assembly, a conductor and an electrode.

6832. The method for inhibiting scarring of any one of items 6597-6830 wherein the electrical lead is unipolar.

6833. The method for inhibiting scarring of any one of items 6597-6830 wherein the electrical lead is bipolar.

6834. The method for inhibiting scarring of any one of items 6597-6830 wherein the electrical lead is tripolar.

6835. The method for inhibiting scarring of any one of items 6597-6830 wherein the electrical lead is quadripolar.

6836. The method for inhibiting scarring of any one of items 6597-6830 wherein the electrical lead comprises an insulating sheath.

6837. The method for inhibiting scarring of any one of items 6597-6830 wherein the electrical lead is a medical lead.

6838. The method for inhibiting scarring of any one of items 6597-6830 wherein the electrical lead is a cardiac lead.

6839. The method for inhibiting scarring of any one of items 6597-6830 wherein the electrical lead is a pacer lead.

6840. The method for inhibiting scarring of any one of items 6597-6830 wherein the electrical lead is a pacing lead.

6841. The method for inhibiting scarring of any one of items 6597-6830 wherein the electrical lead is a pacemaker lead.

6842. The method for inhibiting scarring of any one of items 6597-6830 wherein the electrical lead is an endocardial lead.

6843. The method for inhibiting scarring of any one of items 6597-6830 wherein the electrical lead is an endocardial pacing lead.

6844. The method for inhibiting scarring of any one of items 6597-6830 wherein the electrical lead is a cardioversion lead.

6845. The method for inhibiting scarring of any one of items 6597-6830 wherein the electrical lead is an epicardial lead.

6846. The method for inhibiting scarring of any one of items 6597-6830 wherein the electrical lead is an epicardial defibrillator lead.

6847. The method for inhibiting scarring of any one of items 6597-6830 wherein the electrical lead is a patch defibrillator.

6848. The method for inhibiting scarring of any one of items 6597-6830 wherein the electrical lead is a patch lead.

6849. The method for inhibiting scarring of any one of items 6597-6830 wherein the electrical lead is an electrical patch.

6850. The method for inhibiting scarring of any one of items 6597-6830 wherein the electrical lead is a transvenous lead.

6851. The method for inhibiting scarring of any one of items 6597-6830 wherein the electrical lead is an active fixation lead.

6852. The method for inhibiting scarring of any one of items 6597-6830 wherein the electrical lead is a passive fixation lead.

6853. The method for inhibiting scarring of any one of items 6597-6830 wherein the electrical lead is a sensing lead.

6854. The method for inhibiting scarring of any one of items 6597-6830 wherein the electrical lead is expandable.

6855. The method for inhibiting scarring of any one of items 6597-6830 wherein the electrical lead has a coil configuration.

6856. The method for inhibiting scarring of any one of items 6597-6830 wherein the electrical lead has an active fixation element for attachment to host tissue.

6857. A method for inhibiting scarring comprising placing a neurostimulator (i.e., an electrical device) and an anti-scarring agent or a composition comprising an ant-scarring agent into an animal host, wherein the agent inhibits scarring.

6858. The method of item 6857 wherein the agent is an adensosine A2A receptor antagonist.

6859. The method of item 6857 wherein the agent is an AKT inhibitor.

6860. The method of item 6857 wherein the agent is an alpha 2 integrin antagonist, wherein the alpha 2 integrin antagonist is Pharmaprojects No. 5754 (Merck KgaA).

6861. The method of item 6857 wherein the agent is an alpha 4 integrin antagonist.

6862. The method of item 6857 wherein the agent is an alpha 7 nicotinic receptor agonist.

6863. The method of item 6857 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

6864. The method of item 6857 wherein the agent is an apoptosis antagonist

6865. The method of item 6857 wherein the agent is an apoptosis activator.

6866. The method of item 6857 wherein the agent is a beta 1 integrin antagonist.

6867. The method of item 6857 wherein the agent is a beta tubulin inhibitor.

6868. The method of item 6857 wherein the agent is a blocker of enzyme production in Hepatitis C.

6869. The method of item 6857 wherein the agent is a Bruton's tyrosine kinase inhibitor.

6870. The method of item 6857 wherein the agent is a calcineurin inhibitor.

6871. The method of item 6857 wherein the agent is a caspase 3 inhibitor.

6872. The method of item 6857 wherein the agent is a CC chemokine receptor antagonist.

6873. The method of item 6857 wherein the agent is a cell cycle inhibitor selected from the group consisting of SNS-595 (Sunesis), synthadotin, KRX-0403, and an analogue or derivative thereof.

6874. The method of item 6857 wherein the agent is a cathepsin B inhibitor.

6875. The method of item 6857 wherein the agent is a cathepsin K inhibitor, wherein the cathepsin K inhibitor is 462795 (GlaxoSmithKline), INPL-022-D6 (Amura Therapeutics), or an analogue or derivative thereof.

6876. The method of item 6857 wherein the agent is a cathepsin L inhibitor.

6877. The method of item 6857 wherein the agent is a CD40 antagonist.

6878. The method of item 6857 wherein the agent is a chemokine receptor agonist.

6879. The method of item 6857 wherein the agent is a chymase inhibitor.

6880. The method of item 6857 wherein the agent is a collagenase antagonist.

6881. The method of item 6857 wherein the agent is a CXCR antagonist.

6882. The method of item 6857 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

6883. The method of item 6857 wherein the agent is a cyclooxygenase 1 inhibitor.

6884. The method of item 6857 wherein the agent is a DHFR inhibitor.

6885. The method of item 6857 wherein the agent is a dual integrin inhibitor.

6886. The method of item 6857 wherein the agent is an elastase inhibitor.

6887. The method of item 6857 wherein the agent is an elongation factor-1 alpha inhibitor.

6888. The method of item 6857 wherein the agent is an endothelial growth factor antagonist.

6889. The method of item 6857 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

6890. The method of item 6857 wherein the agent is an endotoxin antagonist.

6891. The method of item 6857 wherein the agent is an epothilone and tubulin binder.

6892. The method of item 6857 wherein the agent is an estrogen receptor antagonist.

6893. The method of item 6857 wherein the agent is an FGF inhibitor

6894. The method of item 6857 wherein the agent is a farnexyl transferase inhibitor.

6895. The method of item 6857 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

6896. The method of item 6857 wherein the agent is an FLT-3 kinase inhibitor.

6897. The method of item 6857 wherein the agent is an FGF receptor kinase inhibitor.

6898. The method of item 6857 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

6899. The method of item 6857 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

6900. The method of item 6857 wherein the agent is a histone deacetylase inhibitor.

6901. The method of item 6857 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

6902. The method of item 6857 wherein the agent is an ICAM inhibitor.

6903. The method of item 6857 wherein the agent is an IL, ICE and IRAK antagonist, wherein the antagonist is a CJ-14877, CP-424174 (Pfizer), NF-61 (Negma-Lerads), and an analogue or derivative thereof.

6904. The method of item 6857 wherein the agent is an IL-2 inhibitor.

6905. The method of item 6857 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

6906. The method of item 6857 wherein the agent is an IMPDH (inosine monophosphate).

6907. The method of item 6857 wherein the agent is an integrin antagonist.

6908. The method of item 6857 wherein the agent is an interleukin antagonist.

6909. The method of item 6857 wherein the agent is an inhibitor of type III receptor tyrosine kinase.

6910. The method of item 6857 wherein the agent is an irreversible inhibitor of enzyme methionine aminopeptidase type 2.

6911. The method of item 6857 wherein the agent is an isozyme selective delta protein kinase C inhibitor.

6912. The method of item 6857 wherein the agent a JAK3 enzyme inhibitor.

6913. The method of item 6857 wherein the agent is a JNK inhibitor.

6914. The method of item 6857 wherein the agent is a kinase inhibitor.

6915. The method of item 6857 wherein the agent is kinesin antagonist.

6916. The method of item 6857 wherein the agent is a kinesin antagonist.

6917. The method of item 6857 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

6918. The method of item 6857 wherein the agent is an MAP kinase inhibitor.

6919. The method of item 6857 wherein the agent is a matrix metalloproteinase inhibitor.

6920. The method of item 6857 wherein the agent is an MCP-CCR2 inhibitor.

6921. The method of item 6857 wherein the agent is an mTOR inhibitor.

6922. The method of item 6857 wherein the agent is an mTOR kinase inhibitor.

6923. The method of item 6857 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

6924. The method of item 6857 wherein the agent is an MIF inhibitor.

6925. The method of item 6857 wherein the agent is an MMP inhibitor.

6926. The method of item 6857 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

6927. The method of item 6857 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

6928. The method of item 6857 wherein the agent is a nitric oxide agonist.

6929. The method of item 6857 wherein the agent is an ornithine decarboxylase inhibitor.

6930. The method of item 6857 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

6931. The method of item 6857 wherein the agent is a palmitoyl-protein thioesterase inhibitor.

6932. The method of item 6857 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

6933. The method of item 6857 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

6934. The method of item 6857 wherein the agent is a phosphatase inhibitor.

6935. The method of item 6857 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

6936. The method of item 6857 wherein the agent is a PKC inhibitor.

6937. The method of item 6857 wherein the agent is a platelet activating factor antagonist.

6938. The method of item 6857 wherein the agent is a platelet-derived growth factor receptor kinase inhibitor.

6939. The method of item 6857 wherein the agent is a prolyl hydroxylase inhibitor.

6940. The method of item 6857 wherein the agent is a polymorphonuclear neutrophil inhibitor.

6941. The method of item 6857 wherein the agent is a protein kinase B inhibitor.

6942. The method of item 6857 wherein the agent is a protein kinase C stimulant.

6943. The method of item 6857 wherein the agent is a purine nucleoside analogue.

6944. The method of item 6857 wherein the agent is a purinoreceptor P2X antagonist.

6945. The method of item 6857 wherein the agent is a Raf kinase inhibitor.

6946. The method of item 6857 wherein the agent is a reversible inhibitor of ErbB1 and ErbB2.

6947. The method of item 6857 wherein the agent is a ribonucleoside triphosphate reductase inhibitor.

6948. The method of item 6857 wherein the agent is an SDF-1 antagonist.

6949. The method of item 6857 wherein the agent is a sheddase inhibitor.

6950. The method of item 6857 wherein the agent is an SRC inhibitor.

6951. The method of item 6857 wherein the agent is a stromelysin inhibitor.

6952. The method of item 6857 wherein the agent is an Syk kinase inhibitor.

6953. The method of item 6857 wherein the agent is a telomerase inhibitor.

6954. The method of item 6857 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

6955. The method of item 6857 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9). (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof

6956. The method of item 6857 wherein the agent is a Toll receptor inhibitor.

6957. The method of item 6857 wherein the agent is a tubulin antagonist.

6958. The method of item 6857 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 14920442-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

6959. The method of item 6857 wherein the agent is a VEGF inhibitor.

6960. The method of item 6857 wherein the agent is a vitamin D receptor agonist.

6961. The method of item 6857 wherein the agent is ZD-6474 (an angiogenesis inhibitor).

6962. The method of item 6857 wherein the agent is AP-23573 (an mTOR inhibitor).

6963. The method of item 6857 wherein the agent is synthadotin (a tubulin antagonist).

6964. The method of item 6857 wherein the agent is S-0885 (a collagenase inhibitor).

6965. The method of item 6857 wherein the agent is aplidine (an elongation factor-1 alpha inhibitor).

6966. The method of item 6857 wherein the agent is ixabepilone (an epithilone).

6967. The method of item 6857 wherein the agent is IDN-5390 (an angiogenesis inhibitor).

6968. The method of item 6857 wherein the agent is SB-2723005 (an angiogenesis inhibitor).

6969. The method of item 6857 wherein the agent is ABT-518 (an angiogenesis inhibitor).

6970. The method of item 6857 wherein the agent is combretastatin (an angiogenesis inhibitor).

6971. The method of item 6857 wherein the agent is anecortave acetate (an angiogenesis inhibitor).

6972. The method of item 6857 wherein the agent is SB-715992 (a kinesin antagonist).

6973. The method of item 6857 wherein the agent is temsirolimus (an mTOR inhibitor).

6974. The method of item 6857 wherein the agent is adalimumab (a TNFα antagonist).

6975. The method of item 6857, wherein the composition comprises a polymer.

6976. The method of item 6857, wherein the composition comprises a polymer, and the polymer is, or comprises, a copolymer.

6977. The method of item 6857, wherein the composition comprises a polymer, and the polymer is, or comprises, a block copolymer.

6978. The method of item 6857, wherein the composition comprises a polymer, and the polymer is, or comprises, a random copolymer.

6979. The method of item 6857, wherein the composition comprises a polymer, and the polymer is, or comprises, a biodegradable polymer.

6980. The method of item 6857, wherein the composition comprises a polymer, and the polymer is, or comprises, a non-biodegradable polymer.

6981. The method of item 6857, wherein the composition comprises a polymer, and the polymer is, or comprises, a hydrophilic polymer.

6982. The method of item 6857, wherein the composition comprises a polymer, and the polymer is, or comprises, a hydrophobic polymer.

6983. The method of item 6857, wherein the composition comprises a polymer, and the polymer is, or comprises, a polymer having hydrophilic domains.

6984. The method of item 6857, wherein the composition comprises a polymer, and the polymer is, or comprises, a polymer having hydrophobic domains.

6985. The method of item 6857, wherein the composition comprises a polymer, and the polymer is, or comprises, a non-conductive polymer.

6986. The method of item 6857, wherein the composition comprises a polymer, and the polymer is, or comprises, an elastomer.

6987. The method of item 6857, wherein the composition comprises a polymer, and the polymer is, or comprises, a hydrogel.

6988. The method of item 6857, wherein the composition comprises a polymer, and the polymer is, or comprises, a silicone polymer.

6989. The method of item 6857, wherein the composition comprises a polymer, and the polymer is, or comprises, a hydrocarbon polymer.

6990. The method of item 6857, wherein the composition comprises a polymer, and the polymer is, or comprises, a styrene-derived polymer.

6991. The method of item 6857, wherein the composition comprises a polymer, and the polymer is, or comprises, a butadiene-derived polymer.

6992. The method of item 6857, wherein the composition comprises a polymer, and the polymer is, or comprises, a macromer.

6993. The method of item 6857, wherein the composition comprises a polymer, and the polymer is, or comprises, a poly(ethylene glycol) polymer.

6994. The method of item 6857, wherein the composition comprises a polymer, and the polymer is, or comprises, an amorphous polymer.

6995. The method of item 6857, wherein the composition further comprises a second pharmaceutically active agent.

6996. The method of item 6857, wherein the composition further comprises an anti-inflammatory agent.

6997. The method of item 6857, wherein the composition further comprises an agent that inhibits infection.

6998. The method of item 6857, wherein the composition further comprises an anthracycline.

6999. The method of item 6857, wherein the composition further comprises doxorubicin.

7000. The method of item 6857 wherein the composition further comprises mitoxantrone.

7001. The method of item 6857 wherein the composition further comprises a fluoropyrimidine.

7002. The method of item 6857, wherein the composition further comprises 5-fluorouracil (5-FU).

7003. The method of item 6857, wherein the composition further comprises a folic acid antagonist.

7004. The method of item 6857, wherein the composition further comprises methotrexate.

7005. The method of item 6857, wherein the composition further comprises a podophylotoxin.

7006. The method of item 6857, wherein the composition further comprises etoposide.

7007. The method of item 6857, wherein the composition further comprises camptothecin.

7008. The method of item 6857, wherein the composition further comprises a hydroxyurea.

7009. The method of item 6857, wherein the composition further comprises a platinum complex.

7010. The method of item 6857, wherein the composition further comprises cisplatin.

7011. The method of item 6857 wherein the composition further comprises an anti-thrombotic agent.

7012. The method of item 6857, wherein the composition further comprises a visualization agent.

7013. The method of item 6857, wherein the composition further comprises a visualization agent, and the visualization agent is a radiopaque material, wherein the radiopaque material comprises a metal, a halogenated compound, or a barium containing compound.

7014. The method of item 6857, wherein the composition further comprises a visualization agent, and the visualization agent is, or comprises, barium, tantalum, or technetium.

7015. The method of item 6857, wherein the composition further comprises a visualization agent, and the visualization agent is, or comprises, an MRI responsive material.

7016. The method of item 6857, wherein the composition further comprises a visualization agent, and the visualization agent is, or comprises, a gadolinium chelate.

7017. The method of item 6857, wherein the composition further comprises a visualization agent, and the visualization agent is, or comprises, iron, magnesium, manganese, copper, or chromium.

7018. The method of item 6857, wherein the composition further comprises a visualization agent, and the visualization agent is, or comprises, iron oxide compound.

7019. The method of item 6857, wherein the composition further comprises a visualization agent, and the visualization agent is, or comprises, a dye, pigment, or colorant.

7020. The method of item 6857 wherein the agent is released in effective concentrations from the composition comprising the agent by diffusion over a period ranging from the time of administration to about 90 days.

7021. The method of item 6857 wherein the agent is released in effective concentrations from the composition comprising the agent by erosion of the composition over a period ranging from the time of administration to about 90 days.

7022. The method of item 6857 wherein the composition further comprises an inflammatory cytokine.

7023. The method of item 6857 wherein the composition further comprises an agent that stimulates cell proliferation.

7024. The method of item 6857 wherein the composition further comprises a polymeric carrier.

7025. The method of item 6857 wherein the composition is in the form of a gel, paste, or spray.

7026. The method of item 6857 wherein the electrical device is partially constructed with the agent or the composition.

7027. The method of item 6857 wherein the electrical device is impregnated with the agent or the composition.

7028. The method of item 6857, wherein the agent or the composition forms a coating, and the coating directly contacts the electrical device.

7029. The method of item 6857, wherein the agent or the composition forms a coating, and the coating indirectly contacts the electrical device.

7030. The method of item 6857 wherein the agent or the composition forms a coating, and the coating partially covers the electrical device.

7031. The method of item 6857, wherein the agent or the composition forms a coating, and the coating completely covers the electrical device.

7032. The method of item 6857 wherein the agent or the composition is located within pores or holes of the electrical device.

7033. The method of item 6857 wherein the agent or the composition is located within a channel, lumen, or divet of the electrical device.

7034. The method of item 6857 wherein the electrical device further comprises an echogenic material.

7035. The method of item 6857 wherein the electrical device further comprises an echogenic material, wherein the echogenic material is in the form of a coating.

7036. The method of item 6857 wherein the electrical device is sterile.

7037. The method of item 6857 wherein the agent is delivered from the electrical device, wherein the agent is released into tissue in the vicinity of the electrical device after deployment of the electrical device.

7038. The method of item 6857 wherein the agent is delivered from the electrical device, wherein the agent is released into tissue in the vicinity of the electrical device after deployment of the electrical device, wherein the tissue is connective tissue.

7039. The method of item 6857 wherein the agent is delivered from the electrical device, wherein the agent is released into tissue in the vicinity of the electrical device after deployment of the electrical device, wherein the tissue is muscle tissue.

7040. The method of item 6857 wherein the agent is delivered from the electrical device, wherein the agent is released into tissue in the vicinity of the electrical device after deployment of the electrical device, wherein the tissue is nerve tissue.

7041. The method of item 6857 wherein the agent is delivered from the electrical device, wherein the agent is released into tissue in the vicinity of the electrical device after deployment of the electrical device, wherein the tissue is epithelium tissue.

7042. The method of item 6857 wherein the agent is delivered from the electrical device, wherein the agent is released in effective concentrations from the electrical device over a period ranging from the time of deployment of the electrical device to about 1 year

7043. The method of item 6857 wherein the agent is delivered from the electrical device, wherein the agent is released in effective concentrations from the electrical device over a period ranging from about 1 month to 6 months.

7044. The method of item 6857 wherein the agent is delivered from the electrical device, wherein the agent is released in effective concentrations from the electrical device over a period ranging from about 1-90 days.

7045. The method of item 6857 wherein the agent is delivered from the electrical device, wherein the agent is released in effective concentrations from the electrical device at a constant rate.

7046. The method of item 6857 wherein the agent is delivered from the electrical device, wherein the agent is released in effective concentrations from the electrical device at an increasing rate.

7047. The method of item 6857 wherein the agent is delivered from the electrical device, wherein the agent is released in effective concentrations from the electrical device at a decreasing rate.

7048. The method of item 6857 wherein the agent is delivered from the electrical device, wherein the electrical device comprises about 0.01 μg to about 10 μg of the agent.

7049. The method of item 6857 wherein the agent is delivered from the electrical device, wherein the electrical device comprises about 10 μg to about 10 mg of the agent.

7050. The method of item 6857 wherein the agent is delivered from the electrical device, wherein the electrical device comprises about 10 mg to about 250 mg of the agent.

7051. The method of item 6857 wherein the agent is delivered from the electrical device, wherein the electrical device comprises about 250 mg to about 1000 mg of the agent.

7052. The method of item 6857 wherein the agent is delivered from the electrical device, wherein the electrical device comprises about 1000 mg to about 2500 mg of the agent.

7053. The method of item 6857 wherein the agent is delivered from the electrical device, wherein a surface of the electrical device comprises less than 0.01 μg of the agent per mm² of electrical device surface to which the agent is applied.

7054. The method of item 6857 wherein the agent is delivered from the electrical device, wherein a surface of the electrical device comprises about 0.01 μg to about 1 μg of the agent per mm² of electrical device surface to which the agent is applied.

7055. The method of item 6857 wherein the agent is delivered from the electrical device, wherein a surface of the electrical device comprises about 1 μg to about 10 μg of the agent per mm² of electrical device surface to which the agent is applied.

7056. The method of item 6857 wherein the agent is delivered from the electrical device, wherein a surface of the electrical device comprises about 10 μg to about 250 μg of the agent per mm² of electrical device surface to which the agent is applied.

7057. The method of item 6857 wherein the agent is delivered from the electrical device, wherein a surface of the electrical device comprises about 250 μg to about 1000 μg of the agent per mm² of electrical device surface to which the agent is applied.

7058. The method of item 6857 wherein the agent is delivered from the electrical device, wherein a surface of the electrical device comprises about 1000 μg to about 2500 μg of the agent per mm² of electrical device surface to which the agent is applied.

7059. The method of item 6857, wherein the electrical device further comprises a coating, and the coating is a uniform coating.

7060. The method of item 6857, wherein the electrical device further comprises a coating, and the coating is a non-uniform coating.

7061. The method of item 6857, wherein the electrical device further comprises a coating, and the coating is a discontinuous coating.

7062. The method of item 6857, wherein the electrical device further comprises a coating, and the coating is a patterned coating.

7063. The method of item 6857, wherein the electrical device further comprises a coating, and the coating has a thickness of 100 μm or less.

7064. The method of item 6857, wherein the electrical device further comprises a coating, and the coating has a thickness of 10 μm or less.

7065. The method of item 6857, wherein the electrical device further comprises a coating, and the coating adheres to the surface of the electrical device upon deployment of the electrical device.

7066. The method of item 6857, wherein the electrical device further comprises a coating, and the coating is stable at room temperature for a period of at least 1 year.

7067. The method of item 6857, wherein the electrical device further comprises a coating, and the agent is present in the coating in an amount ranging between about 0.0001% to about 1% by weight.

7068. The method of item 6857, wherein the electrical device further comprises a coating, and the agent is present in the coating in an amount ranging between about 1% to about 10% by weight.

7069. The method of item 6857, wherein the electrical device further comprises a coating, and the agent is present in the coating in an amount ranging between about 10% to about 25% by weight.

7070. The method of item 6857, wherein the electrical device further comprises a coating, and the agent is present in the coating in an amount ranging between about 25% to about 70% by weight.

7071. The method of item 6857, wherein the electrical device further comprises a coating, and the coating comprises a polymer.

7072. The method of item 6857, wherein the electrical device comprises a first coating having a first composition and a second coating having a second composition.

7073. The method of item 6857, wherein the electrical device comprises a first coating having a first composition and a second coating having a second composition, wherein the first composition and the second composition are different.

7074. The method of item 6857 wherein the agent or the composition is affixed to the electrical device.

7075. The method of item 6857 wherein the agent or the composition is covalently attached to the electrical device.

7076. The method of item 6857 wherein the agent or the composition is non-covalently attached to the electrical device.

7077. The method of item 6857 wherein the electrical device comprises a coating that absorbs the agent or the composition.

7078. The method of item 6857 wherein the electrical device is interweaved with a thread composed of, or coated with, the agent or the composition.

7079. The method of item 6857 wherein a portion of the electrical device is covered with a sleeve that contains the agent or the composition.

7080. The method of item 6857 wherein the electrical device is completely covered with a sleeve that contains the agent or the composition.

7081. The method of item 6857 wherein a portion of the electrical device is covered with a mesh that contains the agent or the composition.

7082. The method of item 6857 wherein the electrical device is completely covered with a mesh that contains the agent or the composition.

7083. The method of item 6857 wherein the agent or the composition is applied to the electrical device surface prior to the placing of the electrical device into the host.

7084. The method of item 6857 wherein the agent or the composition is applied to the electrical device surface during the placing of the electrical device into the host.

7085. The method of item 6857 wherein the agent or the composition is applied to the electrical device surface immediately after the placing of the electrical device into the host.

7086. The method of item 6857 wherein the agent or the composition is applied to the surface of the tissue in the host surrounding the electrical device prior to the placing of the electrical device into the host.

7087. The method of item 6857 wherein the agent or the composition is applied to the surface of the tissue in the host surrounding the electrical device during the placing of the electrical device into the host.

7088. The method of item 6857 wherein the agent or the composition is applied to the surface of the tissue in the host surrounding the electrical device immediately after the placing of the electrical device into the host.

7089. The method of item 6857 wherein the agent or the composition is topically applied into the anatomical space where the electrical device is placed.

7090. The method of item 6857 wherein the agent or the composition is percutaneously injected into the tissue in the host surrounding the electrical device.

7091. The method for inhibiting scarring of any one of items 6857-7090 wherein the neurostimulator is a spinal cord stimulator.

7092. The method for inhibiting scarring of any one of items 6857-7090 wherein the neurostimulator is a brain stimulator.

7093. The method for inhibiting scarring of any one of items 6857-7090 wherein the neurostimulator is a vagus nerve stimulator.

7094. The method for inhibiting scarring of any one of items 6857-7090 wherein the neurostimulator is a sacral nerve stimulator.

7095. The method for inhibiting scarring of any one of items 6857-7090 wherein the neurostimulator is a gastric nerve stimulator.

7096. The method for inhibiting scarring of any one of items 6857-7090 wherein the neurostimulator is an auditory nerve stimulator.

7097. The method for inhibiting scarring of any one of items 6857-7090 wherein the neurostimulator delivers stimulation to organs.

7098. The method for inhibiting scarring of any one of items 6857-7090 wherein the neurostimulator delivers stimulation to bone.

7099. The method for inhibiting scarring of any one of items 6857-7090 wherein the neurostimulator delivers stimulation to muscles.

7100. The method for inhibiting scarring of any one of items 6857-7090 wherein the neurostimulator delivers stimulation to tissues.

7101. The method for inhibiting scarring of any one of items 6857-7090 wherein the neurostimulator is a device for continuous subarachnoid infusion.

7102. The method for inhibiting scarring of any one of items 6857-7090 wherein the neurostimulator is an implantable electrode.

7103. The method for inhibiting scarring of any one of items 6857-7090 wherein the neurostimulator is an electrical lead.

7104. The method for inhibiting scarring of any one of items 6857-7090 wherein the neurostimulator is a simulation catheter lead.

7105. The method for inhibiting scarring of any one of items 6857-7090 wherein the neurostimulator is cochlear implant.

7106. The method for inhibiting scarring of any one of items 6857-7090 wherein the neurostimulator is a microstimulator.

7107. The method for inhibiting scarring of any one of items 6857-7090 wherein the neurostimulator is battery powered.

7108. The method for inhibiting scarring of any one of items 6857-7090 wherein the neurostimulator is radio frequency powered.

7109. The method for inhibiting scarring of any one of items 6857-7090 wherein the neurostimulator is both battery and radio frequency powered.

7110. The method for inhibiting scarring of any one of items 6857-7090 wherein the neurostimulator is adapted for treating or preventing pain.

7111. The method for inhibiting scarring of any one of items 6857-7090 wherein the neurostimulator is adapted for treating or preventing epilepsy.

7112. The method for inhibiting scarring of any one of items 6857-7090 wherein the neurostimulator is adapted for treating or preventing Parkinson's disease.

7113. The method for inhibiting scarring of any one of items 6857-7090 wherein the neurostimulator is adapted for treating or preventing movement disorders.

7114. The method for inhibiting scarring of any one of items 6857-7090 wherein the neurostimulator is adapted for treating or preventing obesity.

7115. The method for inhibiting scarring of any one of items 6857-7090 wherein the neurostimulator is adapted for treating or preventing depression.

7116. The method for inhibiting scarring of any one of items 6857-7090 wherein the neurostimulator is adapted for treating or preventing anxiety.

7117. The method for inhibiting scarring of any one of items 6857-7090 wherein the neurostimulator is adapted for treating or preventing hearing loss.

7118. The method for inhibiting scarring of any one of items 6857-7090 wherein the neurostimulator is adapted for treating or preventing ulcers.

7119. The method for inhibiting scarring of any one of items 6857-7090 wherein the neurostimulator is adapted for treating or preventing deep vein thrombosis.

7120. The method for inhibiting scarring of any one of items 6857-7090 wherein the neurostimulator is adapted for treating or preventing muscular atrophy.

7121. The method for inhibiting scarring of any one of items 6857-7090 wherein the neurostimulator is adapted for treating or preventing joint stiffness.

7122. The method for inhibiting scarring of any one of items 6857-7090 wherein the neurostimulator is adapted for treating or preventing muscle spasms.

7123. The method for inhibiting scarring of any one of items 6857-7090 wherein the neurostimulator is adapted for treating or preventing osteoporosis.

7124. The method for inhibiting scarring of any one of items 6857-7090 wherein the neurostimulator is adapted for treating or preventing scoliosis.

7125. The method for inhibiting scarring of any one of items 6857-7090 wherein the neurostimulator is adapted for treating or preventing spinal disc degeneration.

7126. The method for inhibiting scarring of any one of items 6857-7090 wherein the neurostimulator is adapted for treating or preventing spinal cord injury.

7127. The method for inhibiting scarring of any one of items 6857-7090 wherein the neurostimulator is adapted for treating or preventing urinary dysfunction.

7128. The method for inhibiting scarring of any one of items 6857-7090 wherein the neurostimulator is adapted for treating or preventing gastroparesis.

7129. The method for inhibiting scarring of any one of items 6857-7090 wherein the neurostimulator is adapted for treating or preventing malignancy.

7130. The method for inhibiting scarring of any one of items 6857-7090 wherein the neurostimulator is adapted for treating or preventing arachnoiditis.

7131. The method for inhibiting scarring of any one of items 6857-7090 wherein the neurostimulator is adapted for treating or preventing chronic disease.

7132. The method for inhibiting scarring of any one of items 6857-7090 wherein the neurostimulator is adapted for treating or preventing migraine.

7133. The method for inhibiting scarring of any one of items 6857-7090 wherein the neurostimulator is adapted for treating or preventing sleep disorders.

7134. The method for inhibiting scarring of any one of items 6857-7090 wherein the neurostimulator is adapted for treating or preventing dementia.

7135. The method for inhibiting scarring of any one of items 6857-7090 wherein the neurostimulator is adapted for treating or preventing Alzheimer's disease.

7136. A method for inhibiting scarring comprising placing a cardiac rhythm management device (i.e., an electrical device) and an anti-scarring agent or a composition comprising an anti-scarring agent into an animal host, wherein the agent inhibits scarring.

7137. The method of item 7136 wherein the agent is an adensosine A2A receptor antagonist.

7138. The method of item 7136 wherein the agent is an AKT inhibitor.

7139. The method of item 7136 wherein the agent is an alpha 2 integrin antagonist, wherein the alpha 2 integrin antagonist is Pharmaprojects No. 5754 (Merck KgaA).

7140. The method of item 7136 wherein the agent is an alpha 4 integrin antagonist.

7141. The method of item 7136 wherein the agent is an alpha 7 nicotinic receptor agonist.

7142. The method of item 7136 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

7143. The method of item 7136 wherein the agent is an apoptosis antagonist.

7144. The method of item 7136 wherein the agent is an apoptosis activator.

7145. The method of item 7136 wherein the agent is a beta 1 integrin antagonist.

7146. The method of item 7136 wherein the agent is a beta tubulin inhibitor.

7147. The method of item 7136 wherein the agent is a blocker of enzyme production in Hepatitis C.

7148. The method of item 7136 wherein the agent is a Bruton's tyrosine kinase inhibitor.

7149. The method of item 7136 wherein the agent is a calcineurin inhibitor.

7150. The method of item 7136 wherein the agent is a caspase 3 inhibitor.

7151. The method of item 7136 wherein the agent is a CC chemokine receptor antagonist.

7152. The method of item 7136 wherein the agent is a cell cycle inhibitor selected from the group consisting of SNS-595 (Sunesis), synthadotin, KRX-0403, and an analogue or derivative thereof.

7153. The method of item 7136 wherein the agent is a cathepsin B inhibitor.

7154. The method of item 7136 wherein the agent is a cathepsin K inhibitor, wherein the cathepsin K inhibitor is 462795 (GlaxoSmithKline), INPL-022-D6 (Amura Therapeutics), or an analogue or derivative thereof.

7155. The method of item 7136 wherein the agent is a cathepsin L inhibitor.

7156. The method of item 7136 wherein the agent is a CD40 antagonist.

7157. The method of item 7136 wherein the agent is a chemokine receptor agonist.

7158. The method of item 7136 wherein the agent is a chymase inhibitor.

7159. The method of item 7136 wherein the agent is a collagenase antagonist.

7160. The method of item 7136 wherein the agent is a CXCR antagonist.

7161. The method of item 7136 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

7162. The method of item 7136 wherein the agent is a cyclooxygenase 1 inhibitor.

7163. The method of item 7136 wherein the agent is a DHFR inhibitor.

7164. The method of item 7136 wherein the agent is a dual integrin inhibitor.

7165. The method of item 7136 wherein the agent is an elastase inhibitor.

7166. The method of item 7136 wherein the agent is an elongation factor-1 alpha inhibitor.

7167. The method of item 7136 wherein the agent is an endothelial growth factor antagonist.

7168. The method of item 7136 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

7169. The method of item 7136 wherein the agent is an endotoxin antagonist.

7170. The method of item 7136 wherein the agent is an epothilone and tubulin binder.

7171. The method of item 7136 wherein the agent is an estrogen receptor antagonist.

7172. The method of item 7136 wherein the agent is an FGF inhibitor.

7173. The method of item 7136 wherein the agent is a farnexyl transferase inhibitor.

7174. The method of item 7136 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

7175. The method of item 7136 wherein the agent is an FLT-3 kinase inhibitor.

7176. The method of item 7136 wherein the agent is an FGF receptor kinase inhibitor.

7177. The method of item 7136 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

7178. The method of item 7136 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

7179. The method of item 7136 wherein the agent is a histone deacetylase inhibitor.

7180. The method of item 7136 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

7181. The method of item 7136 wherein the agent is an ICAM inhibitor.

7182. The method of item 7136 wherein the agent is an IL, ICE and IRAK antagonist, wherein the antagonist is a CJ-14877, CP-424174 (Pfizer), NF-61 (Negma-Lerads), and an analogue or derivative thereof.

7183. The method of item 7136 wherein the agent is an IL-2 inhibitor.

7184. The method of item 7136 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

7185. The method of item 7136 wherein the agent is an IMPDH (inosine monophosphate).

7186. The method of item 7136 wherein the agent is an integrin antagonist.

7187. The method of item 7136 wherein the agent is an interleukin antagonist.

7188. The method of item 7136 wherein the agent is an inhibitor of type III receptor tyrosine kinase.

7189. The method of item 7136 wherein the agent is an irreversible inhibitor of enzyme methionine aminopeptidase type 2.

7190. The method of item 7136 wherein the agent is an isozyme selective delta protein kinase C inhibitor.

7191. The method of item 7136 wherein the agent a JAK3 enzyme inhibitor.

7192. The method of item 7136 wherein the agent is a JNK inhibitor.

7193. The method of item 7136 wherein the agent is a kinase inhibitor.

7194. The method of item 7136 wherein the agent is kinesin antagonist.

7195. The method of item 7136 wherein the agent is a kinesin antagonist.

7196. The method of item 7136 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (GAS No. 85197-77-9) (Bristol-Myers Squibb), U-75302 (CAS No. 119477-85-9) (Pfizer), and analogue or derivative thereof.

7197. The method of item 7136 wherein the agent is an MAP kinase inhibitor.

7198. The method of item 7136 wherein the agent is a matrix metalloproteinase inhibitor.

7199. The method of item 7136 wherein the agent is an MCP-CCR2 inhibitor.

7200. The method of item 7136 wherein the agent is an mTOR inhibitor.

7201. The method of item 7136 wherein the agent is an mTOR kinase inhibitor.

7202. The method of item 7136 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

7203. The method of item 7136 wherein the agent is an MIF inhibitor.

7204. The method of item 7136 wherein the agent is an MMP inhibitor.

7205. The method of item 7136 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

7206. The method of item 7136 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

7207. The method of item 7136 wherein the agent is a nitric oxide agonist.

7208. The method of item 7136 wherein the agent is an ornithine decarboxylase inhibitor.

7209. The method of item 7136 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

7210. The method of item 7136 wherein the agent is a palmitoyl-protein thioesterase inhibitor.

7211. The method of item 7136 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

7212. The method of item 7136 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

7213. The method of item 7136 wherein the agent is a phosphatase inhibitor.

7214. The method of item 7136 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

7215. The method of item 7136 wherein the agent is a PKC inhibitor.

7216. The method of item 7136 wherein the agent is a platelet activating factor antagonist.

7217. The method of item 7136 wherein the agent is a platelet-derived growth factor receptor kinase inhibitor.

7218. The method of item 7136 wherein the agent is a prolyl hydroxylase inhibitor.

7219. The method of item 7136 wherein the agent is a polymorphonuclear neutrophil inhibitor.

7220. The method of item 7136 wherein the agent is a protein kinase B inhibitor.

7221. The method of item 7136 wherein the agent is a protein kinase C stimulant.

7222. The method of item 7136 wherein the agent is a purine nucleoside analogue.

7223. The method of item 7136 wherein the agent is a purinoreceptor P2X antagonist.

7224. The method of item 7136 wherein the agent is a Raf kinase inhibitor.

7225. The method of item 7136 wherein the agent is a reversible inhibitor of ErbB1 and ErbB2.

7226. The method of item 7136 wherein the agent is a ribonucleoside triphosphate reductase inhibitor.

7227. The method of item 7136 wherein the agent is an SDF-1 antagonist.

7228. The method of item 7136 wherein the agent is a sheddase inhibitor.

7229. The method of item 7136 wherein the agent is an SRC inhibitor.

7230. The method of item 7136 wherein the agent is a stromelysin inhibitor.

7231. The method of item 7136 wherein the agent is an Syk kinase inhibitor.

7232. The method of item 7136 wherein the agent is a telomerase inhibitor.

7233. The method of item 7136 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

7234. The method of item 7136 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

7235. The method of item 7136 wherein the agent is a Toll receptor inhibitor.

7236. The method of item 7136 wherein the agent is a tubulin antagonist.

7237. The method of item 7136 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

7238. The method of item 7136 wherein the agent is a VEGF inhibitor.

7239. The method of item 7136 wherein the agent is a vitamin D receptor agonist.

7240. The method of item 7136 wherein the agent is ZD-6474 (an angiogenesis inhibitor).

7241. The method of item 7136 wherein the agent is AP-23573 (an mTOR inhibitor).

7242. The method of item 7136 wherein the agent is synthadotin (a tubulin antagonist).

7243. The method of item 7136 wherein the agent is S-0885 (a collagenase inhibitor).

7244. The method of item 7136 wherein the agent is aplidine (an elongation factor-1 alpha inhibitor).

7245. The method of item 7136 wherein the agent is ixabepilone (an epithilone).

7246. The method of item 7136 wherein the agent is IDN-5390 (an angiogenesis inhibitor).

7247. The method of item 7136 wherein the agent is SB-2723005 (an angiogenesis inhibitor).

7248. The method of item 7136 wherein the agent is ABT-518 (an angiogenesis inhibitor).

7249. The method of item 7136 wherein the agent is combretastatin (an angiogenesis inhibitor).

7250. The method of item 7136 wherein the agent is anecortave acetate (an angiogenesis inhibitor).

7251. The method of item 7136 wherein the agent is SB-715992 (a kinesin antagonist).

7252. The method of item 7136 wherein the agent is temsirolimus (an mTOR inhibitor).

7253. The method of item 7136 wherein the agent is adalimumab (a TNFα antagonist).

7254. The method of item 7136, wherein the composition comprises a polymer.

7255. The method of item 7136, wherein the composition comprises a polymer, and the polymer is, or comprises, a copolymer.

7256. The method of item 7136, wherein the composition comprises a polymer, and the polymer is, or comprises, a block copolymer.

7257. The method of item 7136, wherein the composition comprises a polymer, and the polymer is, or comprises, a random copolymer.

7258. The method of item 7136, wherein the composition comprises a polymer, and the polymer is, or comprises, a biodegradable polymer.

7259. The method of item 7136, wherein the composition comprises a polymer, and the polymer is, or comprises, a non-biodegradable polymer.

7260. The method of item 7136, wherein the composition comprises a polymer, and the polymer is, or comprises, a hydrophilic polymer.

7261. The method of item 7136, wherein the composition comprises a polymer, and the polymer is, or comprises, a hydrophobic polymer.

7262. The method of item 7136, wherein the composition comprises a polymer, and the polymer is, or comprises, a polymer having hydrophilic domains.

7263. The method of item 7136, wherein the composition comprises a polymer, and the polymer is, or comprises, a polymer having hydrophobic domains.

7264. The method of item 7136, wherein the composition comprises a polymer, and the polymer is, or comprises, a non-conductive polymer.

7265. The method of item 7136, wherein the composition comprises a polymer, and the polymer is, or comprises, an elastomer.

7266. The method of item 7136, wherein the composition comprises a polymer, and the polymer is, or comprises, a hydrogel.

7267. The method of item 7136, wherein the composition comprises a polymer, and the polymer is, or comprises, a silicone polymer.

7268. The method of item 7136, wherein the composition comprises a polymer, and the polymer is, or comprises, a hydrocarbon polymer.

7269. The method of item 7136, wherein the composition comprises a polymer, and the polymer is, or comprises, a styrene-derived polymer.

7270. The method of item 7136, wherein the composition comprises a polymer, and the polymer is, or comprises, a butadiene-derived polymer.

7271. The method of item 7136, wherein the composition comprises a polymer, and the polymer is, or comprises, a macromer.

7272. The method of item 7136, wherein the composition comprises a polymer, and the polymer is, or comprises, a poly(ethylene glycol) polymer.

7273. The method of item 7136, wherein the composition comprises a polymer, and the polymer is, or comprises, an amorphous polymer.

7274. The method of item 7136, wherein the composition further comprises a second pharmaceutically active agent.

7275. The method of item 7136, wherein the composition further comprises an anti-inflammatory agent.

7276. The method of item 7136, wherein the composition further comprises an agent that inhibits infection.

7277. The method of item 7136, wherein the composition further comprises an anthracycline.

7278. The method of item 7136, wherein the composition further comprises doxorubicin.

7279. The method of item 7136 wherein the composition further comprises mitoxantrone.

7280. The method of item 7136 wherein the composition further comprises a fluoropyrimidine.

7281. The method of item 7136, wherein the composition further comprises 5-fluorouracil (5-FU).

7282. The method of item 7136, wherein the composition further comprises a folic acid antagonist.

7283. The method of item 7136, wherein the composition further comprises methotrexate.

7284. The method of item 7136, wherein the composition further comprises a podophylotoxin.

7285. The method of item 7136, wherein the composition further comprises etoposide.

7286. The method of item 7136, wherein the composition further comprises camptothecin.

7287. The method of item 7136, wherein the composition further comprises a hydroxyurea.

7288. The method of item 7136, wherein the composition further comprises a platinum complex.

7289. The method of item 7136, wherein the composition further comprises cisplatin.

7290. The method of item 7136 wherein the composition further comprises an anti-thrombotic agent.

7291. The method of item 7136, wherein the composition further comprises a visualization agent.

7292. The method of item 7136, wherein the composition further comprises a visualization agent, and the visualization agent is a radiopaque material, wherein the radiopaque material comprises a metal, a halogenated compound, or a barium containing compound.

7293. The method of item 7136, wherein the composition further comprises a visualization agent, and the visualization agent is, or comprises, barium, tantalum, or technetium.

7294. The method of item 7136, wherein the composition further comprises a visualization agent, and the visualization agent is, or comprises, an MRI responsive material.

7295. The method of item 7136, wherein the composition further comprises a visualization agent, and the visualization agent is, or comprises, a gadolinium chelate.

7296. The method of item 7136, wherein the composition further comprises a visualization agent, and the visualization agent is, or comprises, iron, magnesium, manganese, copper, or chromium.

7297. The method of item 7136, wherein the composition further comprises a visualization agent, and the visualization agent is, or comprises, iron oxide compound.

7298. The method of item 7136, wherein the composition further comprises a visualization agent, and the visualization agent is, or comprises, a dye, pigment, or colorant.

7299. The method of item 7136 wherein the agent is released in effective concentrations from the composition comprising the agent by diffusion over a period ranging from the time of administration to about 90 days.

7300. The method of item 7136 wherein the agent is released in effective concentrations from the composition comprising the agent by erosion of the composition over a period ranging from the time of administration to about 90 days.

7301. The method of item 7136 wherein the composition further comprises an inflammatory cytokine.

7302. The method of item 7136 wherein the composition further comprises an agent that stimulates cell proliferation.

7303. The method of item 7136 wherein the composition further comprises a polymeric carrier.

7304. The method of item 7136 wherein the composition is in the form of a gel, paste, or spray.

7305. The method of item 7136 wherein the electrical device is partially constructed with the agent or the composition.

7306. The method of item 7136 wherein the electrical device is impregnated with the agent or the composition.

7307. The method of item 7136, wherein the agent or the composition forms a coating, and the coating directly contacts the electrical device.

7308. The method of item 7136, wherein the agent or the composition forms a coating, and the coating indirectly contacts the electrical device.

7309. The method of item 7136 wherein the agent or the composition forms a coating, and the coating partially covers the electrical device.

7310. The method of item 7136, wherein the agent or the composition forms a coating, and the coating completely covers the electrical device.

7311. The method of item 7136 wherein the agent or the composition is located within pores or holes of the electrical device.

7312. The method of item 7136 wherein the agent or the composition is located within a channel, lumen, or divet of the electrical device.

7313. The method of item 7136 wherein the electrical device further comprises an echogenic material.

7314. The method of item 7136 wherein the electrical device further comprises an echogenic material, wherein the echogenic material is in the form of a coating.

7315. The method of item 7136 wherein the electrical device is sterile.

7316. The method of item 7136 wherein the agent is delivered from the electrical device, wherein the agent is released into tissue in the vicinity of the electrical device after deployment of the electrical device.

7317. The method of item 7136 wherein the agent is delivered from the electrical device, wherein the agent is released into tissue in the vicinity of the electrical device after deployment of the electrical device, wherein the tissue is connective tissue.

7318. The method of item 7136 wherein the agent is delivered from the electrical device, wherein the agent is released into tissue in the vicinity of the electrical device after deployment of the electrical device, wherein the tissue is muscle tissue.

7319. The method of item 7136 wherein the agent is delivered from the electrical device, wherein the agent is released into tissue in the vicinity of the electrical device after deployment of the electrical device, wherein the tissue is nerve tissue.

7320. The method of item 7136 wherein the agent is delivered from the electrical device, wherein the agent is released into tissue in the vicinity of the electrical device after deployment of the electrical device, wherein the tissue is epithelium tissue.

7321. The method of item 7136 wherein the agent is delivered from the electrical device, wherein the agent is released in effective concentrations from the electrical device over a period ranging from the time of deployment of the electrical device to about 1 year.

7322. The method of item 7136 wherein the agent is delivered from the electrical device, wherein the agent is released in effective concentrations from the electrical device over a period ranging from about 1 month to 6 months.

7323. The method of item 7136 wherein the agent is delivered from the electrical device, wherein the agent is released in effective concentrations from the electrical device over a period ranging from about 1-90 days

7324. The method of item 7136 wherein the agent is delivered from the electrical device, wherein the agent is released in effective concentrations from the electrical device at a constant rate.

7325. The method of item 7136 wherein the agent is delivered from the electrical device, wherein the agent is released in effective concentrations from the electrical device at an increasing rate.

7326. The method of item 7136 wherein the agent is delivered from the electrical device, wherein the agent is released in effective concentrations from the electrical device at a decreasing rate.

7327. The method of item 7136 wherein the agent is delivered from the electrical device, wherein the electrical device comprises about 0.01 μg to about 10 μg of the agent.

7328. The method of item 7136 wherein the agent is delivered from the electrical device, wherein the electrical device comprises about 10 μg to about 10 mg of the agent.

7329. The method of item 7136 wherein the agent is delivered from the electrical device, wherein the electrical device comprises about 10 mg to about 250 mg of the agent.

7330. The method of item 7136 wherein the agent is delivered from the electrical device, wherein the electrical device comprises about 250 mg to about 1000 mg of the agent.

7331. The method of item 7136 wherein the agent is delivered from the electrical device, wherein the electrical device comprises about 1000 mg to about 2500 mg of the agent

7332. The method of item 7136 wherein the agent is delivered from the electrical device, wherein a surface of the electrical device comprises less than 0.01 μg of the agent per mm² of electrical device surface to which the agent is applied.

7333. The method of item 7136 wherein the agent is delivered from the electrical device, wherein a surface of the electrical device comprises about 0.01 μg to about 1 μg of the agent per mm² of electrical device surface to which the agent is applied.

7334. The method of item 7136 wherein the agent is delivered from the electrical device, wherein a surface of the electrical device comprises about 1 μg to about 10 μg of the agent per mm² of electrical device surface to which the agent is applied

7335. The method of item 7136 wherein the agent is delivered from the electrical device, wherein a surface of the electrical device comprises about 10 μg to about 250 μg of the agent per mm² of electrical device surface to which the agent is applied.

7336. The method of item 7136 wherein the agent is delivered from the electrical device, wherein a surface of the electrical device comprises about 250 μg to about 1000 μg of the agent per mm² of electrical device surface to which the agent is applied.

7337. The method of item 7136 wherein the agent is delivered from the electrical device, wherein a surface of the electrical device comprises about 1000 μg to about 2500 μg of the agent per mm² of electrical device surface to which the agent is applied.

7338. The method of item 7136, wherein the electrical device further comprises a coating, and the coating is a uniform coating.

7339. The method of item 7136, wherein the electrical device further comprises a coating, and the coating is a non-uniform coating.

7340. The method of item 7136, wherein the electrical device further comprises a coating, and the coating is a discontinuous coating.

7341. The method of item 7136, wherein the electrical device further comprises a coating, and the coating is a patterned coating.

7342. The method of item 7136, wherein the electrical device further comprises a coating, and the coating has a thickness of 100 μm or less.

7343. The method of item 7136, wherein the electrical device further comprises a coating, and the coating has a thickness of 10 μm or less.

7344. The method of item 7136, wherein the electrical device further comprises a coating, and the coating adheres to the surface of the electrical device upon deployment of the electrical device.

7345. The method of item 7136, wherein the electrical device further comprises a coating, and the coating is stable at room temperature for a period of at least 1 year.

7346. The method of item 7136, wherein the electrical device further comprises a coating, and the agent is present in the coating in an amount ranging between about 0.0001% to about 1% by weight.

7347. The method of item 7136, wherein the electrical device further comprises a coating, and the agent is present in the coating in an amount ranging between about 1% to about 10% by weight.

7348. The method of item 7136, wherein the electrical device further comprises a coating, and the agent is present in the coating in an amount ranging between about 10% to about 25% by weight.

7349. The method of item 7136, wherein the electrical device further comprises a coating, and the agent is present in the coating in an amount ranging between about 25% to about 70% by weight.

7350. The method of item 7136, wherein the electrical device further comprises a coating, and the coating comprises a polymer.

7351. The method of item 7136, wherein the electrical device comprises a first coating having a first composition and a second coating having a second composition.

7352. The method of item 7136, wherein the electrical device comprises a first coating having a first composition and a second coating having a second composition, wherein the first composition and the second composition are different.

7353. The method of item 7136 wherein the agent or the composition is affixed to the electrical device

7354. The method of item 7136 wherein the agent or the composition is covalently attached to the electrical device.

7355. The method of item 7136 wherein the agent or the composition is non-covalently attached to the electrical device.

7356. The method of item 7136 wherein the electrical device comprises a coating that absorbs the agent or the composition.

7357. The method of item 7136 wherein the electrical device is interweaved with a thread composed of, or coated with, the agent or the composition.

7358. The method of item 7136 wherein a portion of the electrical device is covered with a sleeve that contains the agent or the composition.

7359. The method of item 7136 wherein the electrical device is completely covered with a sleeve that contains the agent or the composition.

7360. The method of item 7136 wherein a portion of the electrical device is covered with a mesh that contains the agent or the composition.

7361. The method of item 7136 wherein the electrical device is completely covered with a mesh that contains the agent or the composition.

7362. The method of item 7136 wherein the agent or the composition is applied to the electrical device surface prior to the placing of the electrical device into the host.

7363. The method of item 7136 wherein the agent or the composition is applied to the electrical device surface during the placing of the electrical device into the host.

7364. The method of item 7136 wherein the agent or the composition is applied to the electrical device surface immediately after the placing of the electrical device into the host.

7365. The method of item 7136 wherein the agent or the composition is applied to the surface of the tissue in the host surrounding the electrical device prior to the placing of the electrical device into the host.

7366. The method of item 7136 wherein the agent or the composition is applied to the surface of the tissue in the host surrounding the electrical device during the placing of the electrical device into the host.

7367. The method of item 7136 wherein the agent or the composition is applied to the surface of the tissue in the host surrounding the electrical device immediately after the placing of the electrical device into the host.

7368. The method of item 7136 wherein the agent or the composition is topically applied into the anatomical space where the electrical device is placed.

7369. The method of item 7136 wherein the agent or the composition is percutaneously injected into the tissue in the host surrounding the electrical device.

7370. The method for inhibiting scarring of any one of items 7136-7369 wherein the cardiac rhythm management device is an implantable pulse generator.

7371. The method for inhibiting scarring of any one of items 7136-7369 wherein the cardiac rhythm management device is an electrical lead.

7372. The method for inhibiting scarring of any one of items 7136-7369 wherein the cardiac rhythm management device is a stimulation lead.

7373. The method for inhibiting scarring of any one of items 7136-7369 wherein the cardiac rhythm management device is a simulation catheter lead.

7374. The method for inhibiting scarring of any one of items 7136-7369 wherein the cardiac rhythm management device is a microstimulator.

7375. The method for inhibiting scarring of any one of items 7136-7369 wherein the cardiac rhythm management device is battery powered.

7376. The method for inhibiting scarring of any one of items 7136-7369 wherein the cardiac rhythm management device is radio frequency powered.

7377. The method for inhibiting scarring of any one of items 7136-7369 wherein the cardiac rhythm management device is both battery and radio frequency powered.

7378. The method for inhibiting scarring of any one of items 7136-7369 wherein the cardiac rhythm management device is a cardiac pacemaker.

7379. The method for inhibiting scarring of any one of items 7136-7369 wherein the cardiac rhythm management device is an implantable cardioverter defibrillator system.

7380. The method for inhibiting scarring of any one of items 7136-7369 wherein the cardiac rhythm management device is a cardiac lead.

7381. The method for inhibiting scarring of any one of items 7136-7369 wherein the cardiac rhythm management device is a pacer lead.

7382. The method for inhibiting scarring of any one of items 7136-7369 wherein the cardiac rhythm management device is an endocardial lead.

7383. The method for inhibiting scarring of any one of items 7136-7369 wherein the cardiac rhythm management device is a cardioversion/defibrillator lead.

7384. The method for inhibiting scarring of any one of items 7136-7369 wherein the cardiac rhythm management device is an epicardial lead.

7385. The method for inhibiting scarring of any one of items 7136-7369 wherein the cardiac rhythm management device is an epicardial defibrillator lead.

7386. The method for inhibiting scarring of any one of items 7136-7369 wherein the cardiac rhythm management device is a patch defibrillator.

7387. The method for inhibiting scarring of any one of items 7136-7369 wherein the cardiac rhythm management device is a patch defibrillator lead.

7388. The method for inhibiting scarring of any one of items 7136-7369 wherein the cardiac rhythm management device is an electrical patch.

7389. The method for inhibiting scarring of any one of items 7136-7369 wherein the cardiac rhythm management device is a transvenous lead.

7390. The method for inhibiting scarring of any one of items 7136-7369 wherein the cardiac rhythm management device is an active fixation lead.

7391. The method for inhibiting scarring of any one of items 7136-7369 wherein the cardiac rhythm management device is a passive fixation lead.

7392. The method for inhibiting scarring of any one of items 7136-7369 wherein the cardiac rhythm management device is a sensing lead.

7393. The method for inhibiting scarring of any one of items 7136-7369 wherein the cardiac rhythm management device is a defibrillator.

7394. The method for inhibiting scarring of any one of items 7136-7369 wherein the cardiac rhythm management device is an implantable sensor.

7395. The method for inhibiting scarring of any one of items 7136-7369 wherein the cardiac rhythm management device is a left ventricular assist device.

7396. The method for inhibiting scarring of any one of items 7136-7369 wherein the cardiac rhythm management device is a pulse generator.

7397. The method for inhibiting scarring of any one of items 7136-7369 wherein the cardiac rhythm management device is a patch lead.

7398. The method for inhibiting scarring of any one of items 7136-7369 wherein the cardiac rhythm management device is an electrical patch.

7399. The method for inhibiting scarring of any one of items 7136-7369 wherein the cardiac rhythm management device is a cardiac stimulator.

7400. The method for inhibiting scarring of any one of items 7136-7369 wherein the cardiac rhythm management device is an electrical deviceable sensor.

7401. The method for inhibiting scarring of any one of items 7136-7369 wherein the cardiac rhythm management device is an electrical deviceable pump.

7402. The method for inhibiting scarring of any one of items 7136-7369 wherein the cardiac rhythm management device is a dural patch.

7403. The method for inhibiting scarring of any one of items 7136-7369 wherein the cardiac rhythm management device is a ventricular peritoneal shunt.

7404. The method for inhibiting scarring of any one of items 7136-7369 wherein the cardiac rhythm management device is a ventricular atrial shunt.

7405. The method for inhibiting scarring of any one of items 7136-7369 wherein the cardiac rhythm management device is adapted for treating or preventing epidural fibrosis post-laminectomy.

7406. The method for inhibiting scarring of any one of items 7136-7369 wherein the cardiac rhythm management device is adapted for treating or preventing cardiac rhythm abnormalities.

7407. The method for inhibiting scarring of any one of items 7136-7369 wherein the cardiac rhythm management device is adapted for treating or preventing atrial rhythm abnormalities.

7408. The method for inhibiting scarring of any one of items 7136-7369 wherein the cardiac rhythm management device is adapted for treating or preventing conduction abnormalities.

7409. The method for inhibiting scarring of any one of items 7136-7369 wherein the cardiac rhythm management device is adapted for treating or preventing ventricular rhythm abnormalities.

7410. A method for making a medical device comprising: combining an electrical device and an anti-scarring agent or a composition comprising an anti-scarring agent, wherein the agent inhibits scarring between the device and a host into which the device is implanted.

7411. The method of item 7410 wherein the agent is an adensosine A2A receptor antagonist.

7412. The method of item 7410 wherein the agent is an AKT inhibitor.

7413. The method of item 7410 wherein the agent is an alpha 2 integrin antagonist, wherein the alpha 2 integrin antagonist is Pharmaprojects No. 5754 (Merck KgaA).

7414. The method of item 7410 wherein the agent is an alpha 4 integrin antagonist.

7415. The method of item 7410 wherein the agent is an alpha 7 nicotinic receptor agonist.

7416. The method of item 7410 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

7417. The method of item 7410 wherein the agent is an apoptosis antagonist.

7418. The method of item 7410 wherein the agent is an apoptosis activator.

7419. The method of item 7410 wherein the agent is a beta I integrin antagonist.

7420. The method of item 7410 wherein the agent is a beta tubulin inhibitor.

7421. The method of item 7410 wherein the agent is a blocker of enzyme production in Hepatitis C.

7422. The method of item 7410 wherein the agent is a Bruton's tyrosine kinase inhibitor.

7423. The method of item 7410 wherein the agent is a calcineurin inhibitor.

7424. The method of item 7410 wherein the agent is a caspase 3 inhibitor.

7425. The method of item 7410 wherein the agent is a CC chemokine receptor antagonist.

7426. The method of item 7410 wherein the agent is a cell cycle inhibitor selected from the group consisting of SNS-595 (Sunesis), synthadotin, KRX-0403, and an analogue or derivative thereof.

7427. The method of item 7410 wherein the agent is a cathepsin B inhibitor.

7428. The method of item 7410 wherein the agent is a cathepsin K inhibitor, wherein the cathepsin K inhibitor is 462795 (GlaxoSmithKline), INPL-022-D6 (Amura Therapeutics), or an analogue or derivative thereof.

7429. The method of item 7410 wherein the agent is a cathepsin L inhibitor.

7430. The method of item 7410 wherein the agent is a CD40 antagonist.

7431. The method of item 7410 wherein the agent is a chemokine receptor agonist.

7432. The method of item 7410 wherein the agent is a chymase inhibitor.

7433. The method of item 7410 wherein the agent is a collagenase antagonist.

7434. The method of item 7410 wherein the agent is a CXCR antagonist.

7435. The method of item 7410 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

7436. The method of item 7410 wherein the agent is a cyclooxygenase 1 inhibitor.

7437. The method of item 7410 wherein the agent is a DHFR inhibitor

7438. The method of item 7410 wherein the agent is a dual integrin inhibitor.

7439. The method of item 7410 wherein the agent is an elastase inhibitor.

7440. The method of item 7410 wherein the agent is an elongation factor-1 alpha inhibitor.

7441. The method of item 7410 wherein the agent is an endothelial growth factor antagonist.

7442. The method of item 7410 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

7443. The method of item 7410 wherein the agent is an endotoxin antagonist.

7444. The method of item 7410 wherein the agent is an epothilone and tubulin binder.

7445. The method of item 7410 wherein the agent is an estrogen receptor antagonist.

7446. The method of item 7410 wherein the agent is an FGF inhibitor.

7447. The method of item 7410 wherein the agent is a farnexyl transferase inhibitor.

7448. The method of item 7410 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

7449. The method of item 7410 wherein the agent is an FLT-3 kinase inhibitor.

7450. The method of item 7410 wherein the agent is an FGF receptor kinase inhibitor.

7451. The method of item 7410 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

7452. The method of item 7410 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

7453. The method of item 7410 wherein the agent is a histone deacetylase inhibitor.

7454. The method of item 7410 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

7455. The method of item 7410 wherein the agent is an ICAM inhibitor.

7456. The method of item 7410 wherein the agent is an IL, ICE and IRAK antagonist, wherein the antagonist is a CJ-14877, CP-424174 (Pfizer), NF-61 (Negma-Lerads), and an analogue or derivative thereof.

7457. The method of item 7410 wherein the agent is an IL-2 inhibitor.

7458. The method of item 7410 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

7459. The method of item 7410 wherein the agent is an IMPDH (inosine monophosphate).

7460. The method of item 7410 wherein the agent is an integrin antagonist.

7461. The method of item 7410 wherein the agent is an interleukin antagonist.

7462. The method of item 7410 wherein the agent is an inhibitor of type III receptor tyrosine kinase.

7463. The method of item 7410 wherein the agent is an irreversible inhibitor of enzyme methionine aminopeptidase type 2.

7464. The method of item 7410 wherein the agent is an isozyme selective delta protein kinase C inhibitor.

7465. The method of item 7410 wherein the agent a JAK3 enzyme inhibitor.

7466. The method of item 7410 wherein the agent is a JNK inhibitor.

7467. The method of item 7410 wherein the agent is a kinase inhibitor.

7468. The method of item 7410 wherein the agent is kinesin antagonist.

7469. The method of item 7410 wherein the agent is a kinesin antagonist.

7470. The method of item 7410 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (GAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

7471. The method of item 7410 wherein the agent is an MAP kinase inhibitor.

7472. The method of item 7410 wherein the agent is a matrix metalloproteinase inhibitor.

7473. The method of item 7410 wherein the agent is an MCP-CCR2 inhibitor.

7474. The method of item 7410 wherein the agent is an mTOR inhibitor.

7475. The method of item 7410 wherein the agent is an mTOR kinase inhibitor.

7476. The method of item 7410 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

7477. The method of item 7410 wherein the agent is an MIF inhibitor.

7478. The method of item 7410 wherein the agent is an MMP inhibitor.

7479. The method of item 7410 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

7480. The method of item 7410 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

7481. The method of item 7410 wherein the agent is a nitric oxide agonist.

7482. The method of item 7410 wherein the agent is an ornithine decarboxylase inhibitor.

7483. The method of item 7410 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

7484. The method of item 7410 wherein the agent is a palmitoyl-protein thioesterase inhibitor.

7485. The method of item 7410 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

7486. The method of item 7410 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

7487. The method of item 7410 wherein the agent is a phosphatase inhibitor.

7488. The method of item 7410 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

7489. The method of item 7410 wherein the agent is a PKC inhibitor.

7490. The method of item 7410 wherein the agent is a platelet activating factor antagonist.

7491. The method of item 7410 wherein the agent is a platelet-derived growth factor receptor kinase inhibitor.

7492. The method of item 7410 wherein the agent is a prolyl hydroxylase inhibitor.

7493. The method of item 7410 wherein the agent is a polymorphonuclear neutrophil inhibitor.

7494. The method of item 7410 wherein the agent is a protein kinase B inhibitor.

7495. The method of item 7410 wherein the agent is a protein kinase C stimulant.

7496. The method of item 7410 wherein the agent is a purine nucleoside analogue.

7497. The method of item 7410 wherein the agent is a purinoreceptor P2X antagonist.

7498. The method of item 7410 wherein the agent is a Raf kinase inhibitor.

7499. The method of item 7410 wherein the agent is a reversible inhibitor of ErbB1 and ErbB2.

7500. The method of item 7410 wherein the agent is a ribonucleoside triphosphate reductase inhibitor.

7501. The method of item 7410 wherein the agent is an SDF-1 antagonist.

7502. The method of item 7410 wherein the agent is a sheddase inhibitor.

7503. The method of item 7410 wherein the agent is an SRC inhibitor.

7504. The method of item 7410 wherein the agent is a stromelysin inhibitor.

7505. The method of item 7410 wherein the agent is an Syk kinase inhibitor.

7506. The method of item 7410 wherein the agent is a telomerase inhibitor.

7507. The method of item 7410 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

7508. The method of item 7410 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

7509. The method of item 7410 wherein the agent is a Toll receptor inhibitor.

7510. The method of item 7410 wherein the agent is a tubulin antagonist.

7511. The method of item 7410 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

7512. The method of item 7410 wherein the agent is a VEGF inhibitor.

7513. The method of item 7410 wherein the agent is a vitamin D receptor agonist.

7514. The method of item 7410 wherein the agent is ZD-6474 (an angiogenesis inhibitor).

7515. The method of item 7410 wherein the agent is AP-23573 (an mTOR inhibitor).

7516. The method of item 7410 wherein the agent is synthadotin (a tubulin antagonist).

7517. The method of item 7410 wherein the agent is S-0885 (a collagenase inhibitor).

7518. The method of item 7410 wherein the agent is aplidine (an elongation factor-1 alpha inhibitor).

7519. The method of item 7410 wherein the agent is ixabepilone (an epithilone).

7520. The method of item 7410 wherein the agent is IDN-5390 (an angiogenesis inhibitor).

7521. The method of item 7410 wherein the agent is SB-2723005 (an angiogenesis inhibitor).

7522. The method of item 7410 wherein the agent is ABT-518 (an angiogenesis inhibitor).

7523. The method of item 7410 wherein the agent is combretastatin (an angiogenesis inhibitor).

7524. The method of item 7410 wherein the agent is anecortave acetate (an angiogenesis inhibitor).

7525. The method of item 7410 wherein the agent is SB-715992 (a kinesin antagonist).

7526. The method of item 7410 wherein the agent is temsirolimus (an mTOR inhibitor).

7527. The method of item 7410 wherein the agent is adalimumab (a TNFα antagonist).

7528. The method of item 7410, wherein the composition comprises a polymer.

7529. The method of item 7410, wherein the composition comprises a polymeric carrier.

7530. The method of item 7410 wherein the anti-scarring agent inhibits adhesion between the medical device and a host into which the medical device is implanted.

7531. The method of item 7410 wherein the medical device delivers the anti-scarring agent locally to tissue proximate to the medical device.

7532. The method of item 7410 wherein the medical device has a coating that comprises the anti-scarring agent.

7533. The method of item 7410, wherein the medical device has a coating that comprises the agent and is disposed on a surface of the electrical device.

7534. The method of item 7410, wherein the medical device has a coating that comprises the agent and directly contacts the electrical device.

7535. The method of item 7410, wherein the medical device has a coating that comprises the agent and indirectly contacts the electrical device.

7536. The method of item 7410, wherein the medical device has a coating that comprises the agent and partially covers the electrical device.

7537. The method of item 7410, wherein the medical device has a coating that comprises the agent and completely covers the electrical device.

7538. The method of item 7410, wherein the medical device has a uniform coating.

7539. The method of item 7410, wherein the medical device has a non-uniform coating.

7540. The method of item 7410, wherein the medical device has a discontinuous coating.

7541. The method of item 7410, wherein the medical device has a patterned coating.

7542. The method of item 7410, wherein the medical device has a coating with a thickness of 100 μm or less.

7543. The method of item 7410, wherein the medical device has a coating with a thickness of 10 μm or less.

7544. The method of item 7410, wherein the medical device has a coating, and the coating adheres to the surface of the electrical device upon deployment of the electrical device.

7545. The method of item 7410, wherein the medical device has a coating, and wherein the coating is stable at room temperature for a period of 1 year.

7546. The method of item 7410, wherein the medical device has a coating, and wherein the anti-scarring agent is present in the coating in an amount ranging between about 0.0001% to about 1% by weight.

7547. The method of item 7410, wherein the medical device has a coating, and wherein the anti-scarring agent is present in the coating in an amount ranging between about 1% to about 10% by weight.

7548. The method of item 7410, wherein the medical device has a coating, and wherein the anti-scarring agent is present in the coating in an amount ranging between about 10% to about 25% by weight.

7549. The method of item 7410, wherein the medical device has a coating, and wherein the anti-scarring agent is present in the coating in an amount ranging between about 25% to about 70% by weight.

7550. The method of item 7410, wherein the medical device has a coating, and wherein the coating further comprises a polymer.

7551. The method of item 7410, wherein the medical device has a first coating having a first composition and a second coating having a second composition.

7552. The method of item 7410, wherein the medical device has a first coating having a first composition and a second coating having a second composition, wherein the first composition and the second composition are different.

7553. The method of item 7410, wherein the composition comprises a polymer.

7554. The method of item 7410, wherein the composition comprises a polymeric carrier.

7555. The method of item 7410, wherein the composition comprises a polymeric carrier, and wherein the polymeric carrier comprises a copolymer.

7556. The method of item 7410, wherein the composition comprises a polymeric carrier, and wherein the polymeric carrier comprises a block copolymer.

7557. The method of item 7410, wherein the composition comprises a polymeric carrier, and wherein the polymeric carrier comprises a random copolymer.

7558. The method of item 7410, wherein the composition comprises a polymeric carrier, and wherein the polymeric carrier comprises a biodegradable polymer

7559. The method of item 7410, wherein the composition comprises a polymeric carrier, and wherein the polymeric carrier comprises a non-biodegradable polymer.

7560. The method of item 7410, wherein the composition comprises a polymeric carrier, and wherein the polymeric carrier comprises a hydrophilic polymer.

7561. The method of item 7410, wherein the composition comprises a polymeric carrier, and wherein the polymeric carrier comprises a hydrophobic polymer.

7562. The method of item 7410, wherein the composition comprises a polymeric carrier, and wherein the polymeric carrier comprises a polymer having hydrophilic domains.

7563. The method of item 7410, wherein the composition comprises a polymeric carrier, and wherein the polymeric carrier comprises a polymer having hydrophobic domains.

7564. The method of item 7410, wherein the composition comprises a polymeric carrier, and wherein the polymeric carrier comprises a non-conductive polymer.

7565. The method of item 7410, wherein the composition comprises a polymeric carrier, and wherein the polymeric carrier comprises an elastomer.

7566. The method of item 7410, wherein the composition comprises a polymeric carrier, and wherein the polymeric carrier comprises a hydrogel.

7567. The method of item 7410, wherein the composition comprises a polymeric carrier, and wherein the polymeric carrier comprises a silicone polymer.

7568. The method of item 7410, wherein the composition comprises a polymeric carrier, and wherein the polymeric carrier comprises a hydrocarbon polymer.

7569. The method of item 7410, wherein the composition comprises a polymeric carrier, and wherein the polymeric carrier comprises a styrene-derived polymer.

7570. The method of item 7410, wherein the composition comprises a polymeric carrier, and wherein the polymeric carrier comprises a butadiene polymer.

7571. The method of item 7410, wherein the composition comprises a polymeric carrier, and wherein the polymeric carrier comprises a macromer.

7572. The method of item 7410, wherein the composition comprises a polymeric carrier, and wherein the polymeric carrier comprises a poly(ethylene glycol) polymer.

7573. The method of item 7410 wherein the composition comprises a polymeric carrier, and wherein the polymeric carrier comprises an amorphous polymer.

7574. The method of item 7410, wherein the medical device comprises a lubricious coating.

7575. The method of item 7410 wherein the anti-scarring agent is located within pores or holes of the medical device.

7576. The method of item 7410 wherein the anti-scarring agent is located within a channel, lumen, or divet of the medical device.

7577. The method of item 7410, wherein the medical device further comprises a second pharmaceutically active agent.

7578. The method of item 7410 wherein the medical device further comprises an anti-inflammatory agent.

7579. The method of item 7410 wherein the medical device further comprises an agent that inhibits infection.

7580. The method of item 7410 wherein the medical device further comprises an agent that inhibits infection, and wherein the agent is an anthracycline.

7581. The method of item 7410 wherein the medical device further comprises an agent that inhibits infection, and wherein the agent is doxorubicin.

7582. The method of item 7410 wherein the medical device further comprises an agent that inhibits infection, and wherein the agent is mitoxantrone.

7583. The method of item 7410 wherein the medical device further comprises an agent that inhibits infection, and wherein the agent is a fluoropyrimidine.

7584. The method of item 7410 wherein the medical device further comprises an agent that inhibits infection, and wherein the agent is 5-fluorouracil (5-FU).

7585. The method of item 7410 wherein the medical device further comprises an agent that inhibits infection, and wherein the agent is a folic acid antagonist.

7586. The method of item 7410 wherein the medical device further comprises an agent that inhibits infection, and wherein the agent is methotrexate.

7587. The method of item 7410 wherein the medical device further comprises an agent that inhibits infection, and wherein the agent is a podophylotoxin.

7588. The method of item 7410 wherein the medical device further comprises an agent that inhibits infection, and wherein the agent is etoposide.

7589. The method of item 7410 wherein the medical device further comprises an agent that inhibits infection, and wherein the agent is a camptothecin.

7590. The method of item 7410 wherein the medical device further comprises an agent that inhibits infection, and wherein the agent is a hydroxyurea.

7591. The method of item 7410 wherein the medical device further comprises an agent that inhibits infection, and wherein the agent is a platinum complex.

7592. The method of item 7410 wherein the medical device further comprises an agent that inhibits infection, and wherein the agent is cisplatin.

7593. The method of item 7410 wherein the medical device further comprises an anti-thrombotic agent.

7594. The method of item 7410 wherein the medical device further comprises a visualization agent.

7595. The method of item 7410 wherein the medical device further comprises a visualization agent, wherein the visualization agent is a radiopaque material, and wherein the radiopaque material further comprises a metal, a halogenated compound, or a barium containing compound.

7596. The method of item 7410 wherein the medical device further comprises a visualization agent, wherein the visualization agent is a radiopaque material, and wherein the radiopaque material further comprises barium, tantalum, or technetium.

7597. The method of item 7410 wherein the medical device further comprises a visualization agent, and wherein the visualization agent is a MRI responsive material.

7598. The method of item 7410 wherein the medical device further comprises a visualization agent, and wherein the visualization agent further comprises a gadolinium chelate.

7599. The method of item 7410 wherein the medical device further comprises a visualization agent, and wherein the visualization agent further comprises iron, magnesium, manganese, copper, or chromium.

7600. The method of item 7410 wherein the medical device further comprises a visualization agent, and wherein the visualization agent further comprises an iron oxide compound.

7601. The method of item 7410 wherein the medical device further comprises a visualization agent, and wherein the visualization agent further comprises a dye, pigment, or colorant.

7602. The method of item 7410 wherein the medical device further comprises an echogenic material.

7603. The method of item 7410 wherein the medical device further comprises an echogenic material, and wherein the echogenic material is in the form of a coating.

7604. The method of item 7410 wherein the medical device is sterile.

7605. The method of item 7410 wherein the anti-scarring agent is released into tissue in the vicinity of the medical device after deployment of the medical device.

7606. The method of item 7410 wherein the anti-scarring agent is released into tissue in the vicinity of the medical device after deployment of the medical device, and wherein the tissue is connective tissue.

7607. The method of item 7410 wherein the anti-scarring agent is released into tissue in the vicinity of the medical device after deployment of the medical device, and wherein the tissue is muscle tissue.

7608. The method of item 7410 wherein the anti-scarring agent is released into tissue in the vicinity of the medical device after deployment of the medical device, and wherein the tissue is nerve tissue.

7609. The method of item 7410 wherein the anti-scarring agent is released into tissue in the vicinity of the medical device after deployment of the medical device, and wherein the tissue is epithelium tissue.

7610. The method of item 7410 wherein the anti-scarring agent is released in effective concentrations from the medical device over a period ranging from the time of deployment of the medical device to about 1 year.

7611. The method of item 7410 wherein the anti-scarring agent is released in effective concentrations from the medical device over a period ranging from about 1 month to 6 months.

7612. The method of item 7410 wherein the anti-scarring agent is released in effective concentrations from the medical device over a period ranging from about 1-90 days.

7613. The method of item 7410 wherein the anti-scarring agent is released in effective concentrations from the medical device at a constant rate.

7614. The method of item 7410 wherein the anti-scarring agent is released in effective concentrations from the medical device at an increasing rate.

7615. The method of item 7410 wherein the anti-scarring agent is released in effective concentrations from the medical device at a decreasing rate.

7616. The method of item 7410 wherein the anti-scarring agent is released in effective concentrations from the composition comprising the anti-scarring agent by diffusion over a period ranging from the time of deployment of the medical device to about 90 days.

7617. The method of item 7410 wherein the anti-scarring agent is released in effective concentrations from the composition comprising the anti-scarring agent by erosion of the composition over a period ranging from the time of deployment of the medical device to about 90 days.

7618. The method of item 7410 wherein the medical device comprises about 0.01 μg to about 10 μg of the anti-scarring agent.

7619. The method of item 7410 wherein the medical device comprises about 10 μg to about 10 mg of the anti-scarring agent.

7620. The method of item 7410 wherein the medical device comprises about 10 mg to about 250 mg of the anti-scarring agent.

7621. The method of item 7410 wherein the medical device comprises about 250 mg to about 1000 mg of the anti-scarring agent.

7622. The method of item 7410 wherein the medical device comprises about 1000 mg to about 2500 mg of the anti-scarring agent.

7623. The method of item 7410 wherein a surface of the medical device comprises less than 0.01 μg of the anti-scarring agent per mm² of medical device surface to which the anti-scarring agent is applied.

7624. The method of item 7410 wherein a surface of the medical device comprises about 0.01 μg to about 1 μg of the anti-scarring agent per mm² of medical device surface to which the anti-scarring agent is applied.

7625. The method of item 7410 wherein a surface of the medical device comprises about 1 μg to about 10 μg of the anti-scarring agent per mm² of medical device surface to which the anti-scarring agent is applied.

7626. The method of item 7410 wherein a surface of the medical device comprises about 10 μg to about 250 μg of the anti-scarring agent per mm² of medical device surface to which the anti-scarring agent is applied.

7627. The method of item 7410 wherein a surface of the medical device comprises about 250 μg to about 1000 μg of the anti-scarring agent of anti-scarring agent per mm² of medical device surface to which the anti-scarring agent is applied.

7628. The method of item 7410 wherein a surface of the medical device comprises about 1000 μg to about 2500 μg of the anti-scarring agent per mm² of medical device surface to which the anti-scarring agent is applied.

7629. The method of item 7410 wherein the combining is performed by direct affixing the agent or the composition to the electrical device.

7630. The method of item 7410 wherein the combining is performed by spraying the agent or the component onto the electrical device.

7631. The method of item 7410 wherein the combining is performed by electrospraying the agent or the composition onto the electrical device.

7632. The method of item 7410 wherein the combining is performed by dipping the electrical device into a solution comprising the agent or the composition.

7633. The method of item 7410 wherein the combining is performed by covalently attaching the agent or the composition to the electrical device.

7634. The method of item 7410 wherein the combining is performed by non-covalently attaching the agent or the composition to the electrical device.

7635. The method of item 7410 wherein the combining is performed by coating the electrical device with a substance that contains the agent or the composition.

7636. The method of item 7410 wherein the combining is performed by coating the electrical device with a substance that absorbs the agent.

7637. The method of item 7410 wherein the combining is performed by interweaving the electrical device with a thread composed of, or coated with, the agent or the composition.

7638. The method of item 7410 wherein the combining is performed by completely covering the electrical device with a sleeve that contains the agent or the composition.

7639. The method of item 7410 wherein the combining is performed by covering a portion of the electrical device with a sleeve that contains the agent or the composition.

7640. The method of item 7410 wherein the combining is performed by completely covering the electrical device with a cover that contains the agent or the composition.

7641. The method of item 7410 wherein the combining is performed by covering a portion of the electrical device with a cover that contains the agent or the composition.

7642. The method of item 7410 wherein the combining is performed by completely covering the electrical device with an electrospun fabric that contains the agent or the composition.

7643. The method of item 7410 wherein the combining is performed by covering a portion of the electrical device with an electrospun fabric that contains the agent or the composition.

7644. The method of item 7410 wherein the combining is performed by completely covering the electrical device with a mesh that contains the agent or the composition.

7645. The method of item 7410 wherein the combining is performed by covering a portion of the electrical device with a mesh that contains the agent or the composition.

7646. The method of item 7410 wherein the combining is performed by constructing a portion of the electrical device with the agent or the composition.

7647. The method of item 7410 wherein the combining is performed by impregnating the electrical device with the agent or the composition.

7648. The method of item 7410 wherein the combining is performed by constructing a portion of the electrical device from a degradable polymer that releases the agent.

7649. The method of item 7410 wherein the combining is performed by dipping the electrical device into a solution that comprise the agent and an inert solvent for the electrical device.

7650. The method of item 7410 wherein the combining is performed by dipping the electrical device into a solution that comprises the agent and a solvent that will swell the electrical device.

7651. The method of item 7410 wherein the combining is performed by dipping the electrical device into a solution that comprises the agent and a solvent that will dissolve the electrical device.

7652. The method of item 7410 wherein the combining is performed by dipping the electrical device into a solution that comprises the agent, a polymer and an inert solvent for the electrical device.

7653. The method of item 7410 wherein the combining is performed by dipping the electrical device into a solution that comprises the agent, a polymer and a solvent that will swell the electrical device.

7654. The method of item 7410 wherein the combining is performed by dipping the electrical device into a solution that comprises the agent, a polymer and a solvent that will dissolve the electrical device.

7655. The method of item 7410 wherein the combining is performed by spraying the electrical device into a solution that comprises the agent and an inert solvent for the electrical device

7656. The method of item 7410 wherein the combining is performed by spraying the electrical device into a solution that comprises the agent and a solvent that will swell the electrical device.

7657. The method of item 7410 wherein the combining is performed by spraying the electrical device into a solution that comprises the agent and a solvent that will dissolve the electrical device.

7658. The method of item 7410 wherein the combining is performed by spraying the electrical device into a solution that comprises the agent, a polymer and an inert solvent for the electrical device.

7659. The method of item 7410 wherein the combining is performed by spraying the electrical device into a solution that comprises the agent, a polymer and a solvent that will swell the electrical device.

7660. The method of item 7410 wherein the combining is performed by spraying the electrical device into a solution that comprises the agent, a polymer and a solvent that will dissolve the electrical device.

7661. The method for making a medical device of any one of items 7410-7660 wherein the electrical device is a neurostimulator.

7662. The method for making a medical device of any one of items 7410-7660 wherein the electrical device is a spinal cord stimulator.

7663. The method for making a medical device of any one of items 7410-7660 wherein the electrical device is a brain stimulator.

7664. The method for making a medical device of any one of items 7410-7660 wherein the electrical device is a vagus nerve stimulator.

7665. The method for making a medical device of any one of items 7410-7660 wherein the electrical device is a sacral nerve stimulator.

7666. The method for making a medical device of any one of items 7410-7660 wherein the electrical device is a gastric nerve stimulator.

7667. The method for making a medical device of any one of items 7410-7660 wherein the electrical device is an auditory nerve stimulator.

7668. The method for making a medical device of any one of items 7410-7660 wherein the electrical device delivers stimulation to organs.

7669. The method for making a medical device of any one of items 7410-7660 wherein the electrical device delivers stimulation to bone.

7670. The method for making a medical device of any one of items 7410-7660 wherein the electrical device delivers stimulation to muscles.

7671. The method for making a medical device of any one of items 7410-7660 wherein the electrical device delivers stimulation to tissues.

7672. The method for making a medical device of any one of items 7410-7660 wherein the electrical device is a device for continuous subarachnoid infusion.

7673. The method for making a medical device of any one of items 7410-7660 wherein the electrical device is an implantable electrode.

7674. The method for making a medical device of any one of items 7410-7660 wherein the electrical device is an implantable pulse generator.

7675. The method for making a medical device of any one of items 7410-7660 wherein the electrical device is an electrical lead.

7676. The method for making a medical device of any one of items 7410-7660 wherein the electrical device is a stimulation lead.

7677. The method for making a medical device of any one of items 7410-7660 wherein the electrical device is a simulation catheter lead.

7678. The method for making a medical device of any one of items 7410-7660 wherein the electrical device is cochlear implant.

7679. The method for making a medical device of any one of items 7410-7660 wherein the electrical device is a microstimulator.

7680. The method for making a medical device of any one of items 7410-7660 wherein the electrical device is battery powered.

7681. The method for making a medical device of any one of items 7410-7660 wherein the electrical device is radio frequency powered.

7682. The method for making a medical device of any one of items 7410-7660 wherein the electrical device is both battery and radio frequency powered.

7683. The method for making a medical device of any one of items 7410-7660 wherein the electrical device is a cardiac rhythm management device.

7684. The method for making a medical device of any one of items 7410-7660 wherein the electrical device is a cardiac pacemaker.

7685. The method for making a medical device of any one of items 7410-7660 wherein the electrical device is an implantable cardioverter defibrillator system.

7686. The method for making a medical device of any one of items 7410-7660 wherein the electrical device is a cardiac lead.

7687. The method for making a medical device of any one of items 7410-7660 wherein the electrical device is a pacer lead.

7688. The method for making a medical device of any one of items 7410-7660 wherein the electrical device is an endocardial lead.

7689. The method for making a medical device of any one of items 7410-7660 wherein the electrical device is a cardioversion/defibrillator lead.

7690. The method for making a medical device of any one of items 7410-7660 wherein the electrical device is an epicardial lead.

7691. The method for making a medical device of any one of items 7410-7660 wherein the electrical device is an epicardial defibrillator lead.

7692. The method for making a medical device of any one of items 7410-7660 wherein the electrical device is a patch defibrillator.

7693. The method for making a medical device of any one of items 7410-7660 wherein the electrical device is a patch defibrillator lead.

7694. The method for making a medical device of any one of items 7410-7660 wherein the electrical device is an electrical patch.

7695. The method for making a medical device of any one of items 7410-7660 wherein the electrical device is a transvenous lead.

7696. The method for making a medical device of any one of items 7410-7660 wherein the electrical device is an active fixation lead.

7697. The method for making a medical device of any one of items 7410-7660 wherein the electrical device is a passive fixation lead.

7698. The method for making a medical device of any one of items 7410-7660 wherein the electrical device is a sensing lead.

7699. The method for making a medical device of any one of items 7410-7660 wherein the electrical device is a defibrillator.

7700. The method for making a medical device of any one of items 7410-7660 wherein the electrical device is an implantable sensor.

7701. The method for making a medical device of any one of items 7410-7660 wherein the electrical device is a left ventricular assist device.

7702. The method for making a medical device of any one of items 7410-7660 wherein the electrical device is a pulse generator.

7703. The method for making a medical device of any one of items 7410-7660 wherein the electrical device is a patch lead.

7704. The method for making a medical device of any one of items 7410-7660 wherein the electrical device is an electrical patch.

7705. The method for making a medical device of any one of items 7410-7660 wherein the electrical device is a cardiac stimulator.

7706. The method for making a medical device of any one of items 7410-7660 wherein the electrical device is an electrical deviceable sensor.

7707. The method for making a medical device of any one of items 7410-7660 wherein the electrical device is an electrical deviceable pump.

7708. The method for making a medical device of any one of items 7410-7660 wherein the electrical device is a dural patch.

7709. The method for making a medical device of any one of items 7410-7660 wherein the electrical device is a ventricular peritoneal shunt.

7710. The method for making a medical device of any one of items 7410-7660 wherein the electrical device is a ventricular atrial shunt.

7711. The method for making a medical device of any one of items 7410-7660 wherein the electrical device is adapted for treating or preventing epidural fibrosis post-laminectomy.

7712. The method for making a medical device of any one of items 7410-7660 wherein the electrical device is adapted for treating or preventing cardiac rhythm abnormalities.

7713. The method for making a medical device of any one of items 7410-7660 wherein the electrical device is adapted for treating or preventing atrial rhythm abnormalities.

7714. The method for making a medical device of any one of items 7410-7660 wherein the electrical device is adapted for treating or preventing conduction abnormalities.

7715. The method for making a medical device of any one of items 7410-7660 wherein the electrical device is adapted for treating or preventing ventricular rhythm abnormalities.

7716. The method for making a medical device of any one of items 7410-7660 wherein the electrical device is adapted for treating or preventing pain.

7717. The method for making a medical device of any one of items 7410-7660 wherein the electrical device is adapted for treating or preventing epilepsy.

7718. The method for making a medical device of any one of items 7410-7660 wherein the electrical device is adapted for treating or preventing Parkinson's disease.

7719. The method for making a medical device of any one of items 7410-7660 wherein the electrical device is adapted for treating or preventing movement disorders.

7720. The method for making a medical device of any one of items 7410-7660 wherein the electrical device is adapted for treating or preventing obesity.

7721. The method for making a medical device of any one of items 7410-7660 wherein the electrical device is adapted for treating or preventing depression.

7722. The method for making a medical device of any one of items 7410-7660 wherein the electrical device is adapted for treating or preventing anxiety.

7723. The method for making a medical device of any one of items 7410-7660 wherein the electrical device is adapted for treating or preventing hearing loss.

7724. The method for making a medical device of any one of items 7410-7660 wherein the electrical device is adapted for treating or preventing ulcers.

7725. The method for making a medical device of any one of items 7410-7660 wherein the electrical device is adapted for treating or preventing deep vein thrombosis.

7726. The method for making a medical device of any one of items 7410-7660 wherein the electrical device is adapted for treating or preventing muscular atrophy.

7727. The method for making a medical device of any one of items 7410-7660 wherein the electrical device is adapted for treating or preventing joint stiffness.

7728. The method for making a medical device of any one of items 7410-7660 wherein the electrical device is adapted for treating or preventing muscle spasms.

7729. The method for making a medical device of any one of items 7410-7660 wherein the electrical device is adapted for treating or preventing osteoporosis.

7730. The method for making a medical device of any one of items 7410-7660 wherein the electrical device is adapted for treating or preventing scoliosis.

7731. The method for making a medical device of any one of items 7410-7660 wherein the electrical device is adapted for treating or preventing spinal disc degeneration.

7732. The method for making a medical device of any one of items 7410-7660 wherein the electrical device is adapted for treating or preventing spinal cord injury.

7733. The method for making a medical device of any one of items 7410-7660 wherein the electrical device is adapted for treating or preventing urinary dysfunction.

7734. The method for making a medical device of any one of items 7410-7660 wherein the electrical device is adapted for treating or preventing gastroparesis.

7735. The method for making a medical device of any one of items 7410-7660 wherein the electrical device is adapted for treating or preventing malignancy.

7736. The method for making a medical device of any one of items 7410-7660 wherein the electrical device is adapted for treating or preventing arachnoiditis.

7737. The method for making a medical device of any one of items 7410-7660 wherein the electrical device is adapted for treating or preventing chronic disease.

7738. The method for making a medical device of any one of items 7410-7660 wherein the electrical device is adapted for treating or preventing migraine.

7739. The method for making a medical device of any one of items 7410-7660 wherein the electrical device is adapted for treating or preventing sleep disorders.

7740. The method for making a medical device of any one of items 7410-7660 wherein the electrical device is adapted for treating or preventing dementia.

7741. The method for making a medical device of any one of items 7410-7660 wherein the electrical device is adapted for treating or preventing Alzheimer's disease.

7742. A method for making a medical device comprising: combining a neurostimulator for treating chronic pain (i.e. an electrical device) and an anti-scarring agent or a composition comprising an anti-scarring agent, wherein the agent inhibits scarring between the device and a host into which the device is implanted.

7743. The method of item 7742 wherein the agent is an adensosine A2A receptor antagonist.

7744. The method of item 7742 wherein the agent is an AKT inhibitor.

7745. The method of item 7742 wherein the agent is an alpha 2 integrin antagonist, wherein the alpha 2 integrin antagonist is Pharmaprojects No. 5754 (Merck KgaA).

7746. The method of item 7742 wherein the agent is an alpha 4 integrin antagonist.

7747. The method of item 7742 wherein the agent is an alpha 7 nicotinic receptor agonist.

7748. The method of item 7742 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

7749. The method of item 7742 wherein the agent is an apoptosis antagonist.

7750. The method of item 7742 wherein the agent is an apoptosis activator.

7751. The method of item 7742 wherein the agent is a beta 1 integrin antagonist.

7752. The method of item 7742 wherein the agent is a beta tubulin inhibitor.

7753. The method of item 7742 wherein the agent is a blocker of enzyme production in Hepatitis C.

7754. The method of item 7742 wherein the agent is a Bruton's tyrosine kinase inhibitor.

7755. The method of item 7742 wherein the agent is a calcineurin inhibitor.

7756. The method of item 7742 wherein the agent is a caspase 3 inhibitor.

7757. The method of item 7742 wherein the agent is a CC chemokine receptor antagonist.

7758. The method of item 7742 wherein the agent is a cell cycle inhibitor selected from the group consisting of SNS-595 (Sunesis), synthadotin, KRX-0403, and an analogue or derivative thereof.

7759. The method of item 7742 wherein the agent is a cathepsin B inhibitor.

7760. The method of item 7742 wherein the agent is a cathepsin K inhibitor, wherein the cathepsin K inhibitor is 462795 (GlaxoSmithKline), INPL-022-D6 (Amura Therapeutics), or an analogue or derivative thereof.

7761. The method of item 7742 wherein the agent is a cathepsin L inhibitor.

7762. The method of item 7742 wherein the agent is a CD40 antagonist.

7763. The method of item 7742 wherein the agent is a chemokine receptor agonist.

7764. The method of item 7742 wherein the agent is a chymase inhibitor.

7765. The method of item 7742 wherein the agent is a collagenase antagonist.

7766. The method of item 7742 wherein the agent is a CXCR antagonist.

7767. The method of item 7742 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

7768. The method of item 7742 wherein the agent is a cyclooxygenase 1 inhibitor.

7769. The method of item 7742 wherein the agent is a DHFR inhibitor.

7770. The method of item 7742 wherein the agent is a dual integrin inhibitor.

7771. The method of item 7742 wherein the agent is an elastase inhibitor.

7772. The method of item 7742 wherein the agent is an elongation factor-1 alpha inhibitor.

7773. The method of item 7742 wherein the agent is an endothelial growth factor antagonist.

7774. The method of item 7742 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

7775. The method of item 7742 wherein the agent is an endotoxin antagonist.

7776. The method of item 7742 wherein the agent is an epothilone and tubulin binder.

7777. The method of item 7742 wherein the agent is an estrogen receptor antagonist.

7778. The method of item 7742 wherein the agent is an FGF inhibitor.

7779. The method of item 7742 wherein the agent is a farnexyl transferase inhibitor.

7780. The method of item 7742 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

7781. The method of item 7742 wherein the agent is an FLT-3 kinase inhibitor

7782. The method of item 7742 wherein the agent is an FGF receptor kinase inhibitor.

7783. The method of item 7742 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

7784. The method of item 7742 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

7785. The method of item 7742 wherein the agent is a histone deacetylase inhibitor.

7786. The method of item 7742 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

7787. The method of item 7742 wherein the agent is an ICAM inhibitor.

7788. The method of item 7742 wherein the agent is an IL, ICE and IRAK antagonist, wherein the antagonist is a CJ-14877, CP-424174 (Pfizer), NF-61 (Negma-Lerads), and an analogue or derivative thereof.

7789. The method of item 7742 wherein the agent is an IL-2 inhibitor.

7790. The method of item 7742 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

7791. The method of item 7742 wherein the agent is an IMPDH (inosine monophosphate).

7792. The method of item 7742 wherein the agent is an integrin antagonist.

7793. The method of item 7742 wherein the agent is an interleukin antagonist.

7794. The method of item 7742 wherein the agent is an inhibitor of type III receptor tyrosine kinase.

7795. The method of item 7742 wherein the agent is an irreversible inhibitor of enzyme methionine aminopeptidase type 2.

7796. The method of item 7742 wherein the agent is an isozyme selective delta protein kinase C inhibitor.

7797. The method of item 7742 wherein the agent a JAK3 enzyme inhibitor.

7798. The method of item 7742 wherein the agent is a JNK inhibitor.

7799. The method of item 7742 wherein the agent is a kinase inhibitor.

7800. The method of item 7742 wherein the agent is kinesin antagonist.

7801. The method of item 7742 wherein the agent is a kinesin antagonist.

7802. The method of item 7742 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

7803. The method of item 7742 wherein the agent is an MAP kinase inhibitor.

7804. The method of item 7742 wherein the agent is a matrix metalloproteinase inhibitor.

7805. The method of item 7742 wherein the agent is an MCP-CCR2 inhibitor.

7806. The method of item 7742 wherein the agent is an mTOR inhibitor.

7807. The method of item 7742 wherein the agent is an mTOR kinase inhibitor.

7808. The method of item 7742 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

7809. The method of item 7742 wherein the agent is an MIF inhibitor.

7810. The method of item 7742 wherein the agent is an MMP inhibitor.

7811. The method of item 7742 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

7812. The method of item 7742 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

7813. The method of item 7742 wherein the agent is a nitric oxide agonist.

7814. The method of item 7742 wherein the agent is an ornithine decarboxylase inhibitor.

7815. The method of item 7742 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

7816. The method of item 7742 wherein the agent is a palmitoyl-protein thioesterase inhibitor.

7817. The method of item 7742 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

7818. The method of item 7742 wherein the agent is (−)-halofenate (Metabolex), AMG-131 (Amgen), antidiabetics from Japan Tobacco, AZD-4619, AZD-8450, AZD-8677 (AstraZeneca), DRF-1 0945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

7819. The method of item 7742 wherein the agent is a phosphatase inhibitor.

7820. The method of item 7742 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

7821. The method of item 7742 wherein the agent is a PKC inhibitor.

7822. The method of item 7742 wherein the agent is a platelet activating factor antagonist.

7823. The method of item 7742 wherein the agent is a platelet-derived growth factor receptor kinase inhibitor.

7824. The method of item 7742 wherein the agent is a prolyl hydroxylase inhibitor.

7825. The method of item 7742 wherein the agent is a polymorphonuclear neutrophil inhibitor.

7826. The method of item 7742 wherein the agent is a protein kinase B inhibitor.

7827. The method of item 7742 wherein the agent is a protein kinase C stimulant.

7828. The method of item 7742 wherein the agent is a purine nucleoside analogue.

7829. The method of item 7742 wherein the agent is a purinoreceptor P2X antagonist.

7830. The method of item 7742 wherein the agent is a Raf kinase inhibitor.

7831. The method of item 7742 wherein the agent is a reversible inhibitor of ErbB1 and ErbB2

7832. The method of item 7742 wherein the agent is a ribonucleoside triphosphate reductase inhibitor.

7833. The method of item 7742 wherein the agent is an SDF-1 antagonist.

7834. The method of item 7742 wherein the agent is a sheddase inhibitor.

7835. The method of item 7742 wherein the agent is an SRC inhibitor.

7836. The method of item 7742 wherein the agent is a stromelysin inhibitor.

7837. The method of item 7742 wherein the agent is an Syk kinase inhibitor.

7838. The method of item 7742 wherein the agent is a telomerase inhibitor.

7839. The method of item 7742 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

7840. The method of item 7742 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

7841. The method of item 7742 wherein the agent is a Toll receptor inhibitor.

7842. The method of item 7742 wherein the agent is a tubulin antagonist.

7843. The method of item 7742 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-1 3 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

7844. The method of item 7742 wherein the agent is a VEGF inhibitor.

7845. The method of item 7742 wherein the agent is a vitamin D receptor agonist.

7846. The method of item 7742 wherein the agent is ZD-6474 (an angiogenesis inhibitor).

7847. The method of item 7742 wherein the agent is AP-23573 (an mTOR inhibitor).

7848. The method of item 7742 wherein the agent is synthadotin (a tubulin antagonist).

7849. The method of item 7742 wherein the agent is S-0885 (a collagenase inhibitor).

7850. The method of item 7742 wherein the agent is aplidine (an elongation factor-1 alpha inhibitor).

7851. The method of item 7742 wherein the agent is ixabepilone (an epithilone).

7852. The method of item 7742 wherein the agent is IDN-5390 (an angiogenesis inhibitor).

7853. The method of item 7742 wherein the agent is SB-2723005 (an angiogenesis inhibitor).

7854. The method of item 7742 wherein the agent is ABT-518 (an angiogenesis inhibitor).

7855. The method of item 7742 wherein the agent is combretastatin (an angiogenesis inhibitor).

7856. The method of item 7742 wherein the agent is anecortave acetate (an angiogenesis inhibitor).

7857. The method of item 7742 wherein the agent is SB-715992 (a kinesin antagonist).

7858. The method of item 7742 wherein the agent is temsirolimus (an mTOR inhibitor).

7859. The method of item 7742 wherein the agent is adalimumab (a TNFα antagonist).

7860. The method of item 7742, wherein the composition comprises a polymer.

7861. The method of item 7742, wherein the composition comprises a polymeric carrier.

7862. The method of item 7742 wherein the anti-scarring agent inhibits adhesion between the medical device and a host into which the medical device is implanted.

7863. The method of item 7742 wherein the medical device delivers the anti-scarring agent locally to tissue proximate to the medical device.

7864. The method of item 7742 wherein the medical device has a coating that comprises the anti-scarring agent.

7865. The method of item 7742, wherein the medical device has a coating that comprises the agent and is disposed on a surface of the electrical device.

7866. The method of item 7742, wherein the medical device has a coating that comprises the agent and directly contacts the electrical device.

7867. The method of item 7742, wherein the medical device has a coating that comprises the agent and indirectly contacts the electrical device.

7868. The method of item 7742, wherein the medical device has a coating that comprises the agent and partially covers the electrical device.

7869. The method of item 7742, wherein the medical device has a coating that comprises the agent and completely covers the electrical device.

7870. The method of item 7742, wherein the medical device has a uniform coating.

7871. The method of item 7742, wherein the medical device has a non-uniform coating.

7872. The method of item 7742, wherein the medical device has a discontinuous coating.

7873. The method of item 7742, wherein the medical device has a patterned coating.

7874. The method of item 7742, wherein the medical device has a coating with a thickness of 100 μm or less.

7875. The method of item 7742, wherein the medical device has a coating with a thickness of 10 μm or less.

7876. The method of item 7742, wherein the medical device has a coating, and the coating adheres to the surface of the electrical device upon deployment of the electrical device.

7877. The method of item 7742, wherein the medical device has a coating, and wherein the coating is stable at room temperature for a period of 1 year.

7878. The method of item 7742, wherein the medical device has a coating, and wherein the anti-scarring agent is present in the coating in an amount ranging between about 0.0001% to about 1% by weight.

7879. The method of item 7742, wherein the medical device has a coating, and wherein the anti-scarring agent is present in the coating in an amount ranging between about 1% to about 10% by weight.

7880. The method of item 7742, wherein the medical device has a coating, and wherein the anti-scarring agent is present in the coating in an amount ranging between about 10% to about 25% by weight.

7881. The method of item 7742, wherein the medical device has a coating, and wherein the anti-scarring agent is present in the coating in an amount ranging between about 25% to about 70% by weight.

7882. The method of item 7742, wherein the medical device has a coating, and wherein the coating further comprises a polymer.

7883. The method of item 7742, wherein the medical device has a first coating having a first composition and a second coating having a second composition.

7884. The method of item 7742, wherein the medical device has a first coating having a first composition and a second coating having a second composition, wherein the first composition and the second composition are different.

7885. The method of item 7742, wherein the composition comprises a polymer.

7886. The method of item 7742, wherein the composition comprises a polymeric carrier.

7887. The method of item 7742, wherein the composition comprises a polymeric carrier, and wherein the polymeric carrier comprises a copolymer.

7888. The method of item 7742, wherein the composition comprises a polymeric carrier, and wherein the polymeric carrier comprises a block copolymer.

7889. The method of item 7742, wherein the composition comprises a polymeric carrier, and wherein the polymeric carrier comprises a random copolymer.

7890. The method of item 7742, wherein the composition comprises a polymeric carrier, and wherein the polymeric carrier comprises a biodegradable polymer.

7891. The method of item 7742, wherein the composition comprises a polymeric carrier, and wherein the polymeric carrier comprises a non-biodegradable polymer.

7892. The method of item 7742, wherein the composition comprises a polymeric carrier, and wherein the polymeric carrier comprises a hydrophilic polymer.

7893. The method of item 7742, wherein the composition comprises a polymeric carrier, and wherein the polymeric carrier comprises a hydrophobic polymer.

7894. The method of item 7742, wherein the composition comprises a polymeric carrier, and wherein the polymeric carrier comprises a polymer having hydrophilic domains.

7895. The method of item 7742, wherein the composition comprises a polymeric carrier, and wherein the polymeric carrier comprises a polymer having hydrophobic domains.

7896. The method of item 7742, wherein the composition comprises a polymeric carrier, and wherein the polymeric carrier comprises a non-conductive polymer.

7897. The method of item 7742, wherein the composition comprises a polymeric carrier, and wherein the polymeric carrier comprises an elastomer.

7898. The method of item 7742, wherein the composition comprises a polymeric carrier, and wherein the polymeric carrier comprises a hydrogel.

7899. The method of item 7742, wherein the composition comprises a polymeric carrier, and wherein the polymeric carrier comprises a silicone polymer.

7900. The method of item 7742, wherein the composition comprises a polymeric carrier, and wherein the polymeric carrier comprises a hydrocarbon polymer

7901. The method of item 7742, wherein the composition comprises a polymeric carrier, and wherein the polymeric carrier comprises a styrene-derived polymer.

7902. The method of item 7742, wherein the composition comprises a polymeric carrier, and wherein the polymeric carrier comprises a butadiene polymer.

7903. The method of item 7742, wherein the composition comprises a polymeric carrier, and wherein the polymeric carrier comprises a macromer.

7904. The method of item 7742, wherein the composition comprises a polymeric carrier, and wherein the polymeric carrier comprises a poly(ethylene glycol) polymer.

7905. The method of item 7742 wherein the composition comprises a polymeric carrier, and wherein the polymeric carrier comprises an amorphous polymer.

7906. The method of item 7742, wherein the medical device comprises a lubricious coating.

7907. The method of item 7742 wherein the anti-scarring agent is located within pores or holes of the medical device.

7908. The method of item 7742 wherein the anti-scarring agent is located within a channel, lumen, or divet of the medical device.

7909. The method of item 7742, wherein the medical device further comprises a second pharmaceutically active agent.

7910. The method of item 7742 wherein the medical device further comprises an anti-inflammatory agent.

7911. The method of item 7742 wherein the medical device further comprises an agent that inhibits infection.

7912. The method of item 7742 wherein the medical device further comprises an agent that inhibits infection, and wherein the agent is an anthracycline.

7913. The method of item 7742 wherein the medical device further comprises an agent that inhibits infection, and wherein the agent is doxorubicin.

7914. The method of item 7742 wherein the medical device further comprises an agent that inhibits infection, and wherein the agent is mitoxantrone.

7915. The method of item 7742 wherein the medical device further comprises an agent that inhibits infection, and wherein the agent is a fluoropyrimidine.

7916. The method of item 7742 wherein the medical device further comprises an agent that inhibits infection, and wherein the agent is 5-fluorouracil (5-FU).

7917. The method of item 7742 wherein the medical device further comprises an agent that inhibits infection, and wherein the agent is a folic acid antagonist.

7918. The method of item 7742 wherein the medical device further comprises an agent that inhibits infection, and wherein the agent is methotrexate.

7919. The method of item 7742 wherein the medical device further comprises an agent that inhibits infection, and wherein the agent is a podophylotoxin.

7920. The method of item 7742 wherein the medical device further comprises an agent that inhibits infection, and wherein the agent is etoposide.

7921. The method of item 7742 wherein the medical device further comprises an agent that inhibits infection, and wherein the agent is a camptothecin.

7922. The method of item 7742 wherein the medical device further comprises an agent that inhibits infection, and wherein the agent is a hydroxyurea.

7923. The method of item 7742 wherein the medical device further comprises an agent that inhibits infection, and wherein the agent is a platinum complex.

7924. The method of item 7742 wherein the medical device further comprises an agent that inhibits infection, and wherein the agent is cisplatin.

7925. The method of item 7742 wherein the medical device further comprises an anti-thrombotic agent.

7926. The method of item 7742 wherein the medical device further comprises a visualization agent.

7927. The method of item 7742 wherein the medical device further comprises a visualization agent, wherein the visualization agent is a radiopaque material, and wherein the radiopaque material further comprises a metal, a halogenated compound, or a barium containing compound.

7928. The method of item 7742 wherein the medical device further comprises a visualization agent, wherein the visualization agent is a radiopaque material, and wherein the radiopaque material further comprises barium, tantalum, or technetium.

7929. The method of item 7742 wherein the medical device further comprises a visualization agent, and wherein the visualization agent is a MRI responsive material.

7930. The method of item 7742 wherein the medical device further comprises a visualization agent, and wherein the visualization agent further comprises a gadolinium chelate.

7931. The method of item 7742 wherein the medical device further comprises a visualization agent, and wherein the visualization agent further comprises iron, magnesium, manganese, copper, or chromium.

7932. The method of item 7742 wherein the medical device further comprises a visualization agent, and wherein the visualization agent further comprises an iron oxide compound.

7933. The method of item 7742 wherein the medical device further comprises a visualization agent, and wherein the visualization agent further comprises a dye, pigment, or colorant.

7934. The method of item 7742 wherein the medical device further comprises an echogenic material.

7935. The method of item 7742 wherein the medical device further comprises an echogenic material, and wherein the echogenic material is in the form of a coating.

7936. The method of item 7742 wherein the medical device is sterile.

7937. The method of item 7742 wherein the anti-scarring agent is released into tissue in the vicinity of the medical device after deployment of the medical device.

7938. The method of item 7742 wherein the anti-scarring agent is released into tissue in the vicinity of the medical device after deployment of the medical device, and wherein the tissue is connective tissue.

7939. The method of item 7742 wherein the anti-scarring agent is released into tissue in the vicinity of the medical device after deployment of the medical device, and wherein the tissue is muscle tissue.

7940. The method of item 7742 wherein the anti-scarring agent is released into tissue in the vicinity of the medical device after deployment of the medical device, and wherein the tissue is nerve tissue.

7941. The method of item 7742 wherein the anti-scarring agent is released into tissue in the vicinity of the medical device after deployment of the medical device, and wherein the tissue is epithelium tissue.

7942. The method of item 7742 wherein the anti-scarring agent is released in effective concentrations from the medical device over a period ranging from the time of deployment of the medical device to about 1 year.

7943. The method of item 7742 wherein the anti-scarring agent is released in effective concentrations from the medical device over a period ranging from about 1 month to 6 months.

7944. The method of item 7742 wherein the anti-scarring agent is released in effective concentrations from the medical device over a period ranging from about 1-90 days.

7945. The method of item 7742 wherein the anti-scarring agent is released in effective concentrations from the medical device at a constant rate.

7946. The method of item 7742 wherein the anti-scarring agent is released in effective concentrations from the medical device at an increasing rate.

7947. The method of item 7742 wherein the anti-scarring agent is released in effective concentrations from the medical device at a decreasing rate.

7948. The method of item 7742 wherein the anti-scarring agent is released in effective concentrations from the composition comprising the anti-scarring agent by diffusion over a period ranging from the time of deployment of the medical device to about 90 days.

7949. The method of item 7742 wherein the anti-scarring agent is released in effective concentrations from the composition comprising the anti-scarring agent by erosion of the composition over a period ranging from the time of deployment of the medical device to about 90 days.

7950. The method of item 7742 wherein the medical device comprises about 0.01 μg to about 10 μg of the anti-scarring agent.

7951. The method of item 7742 wherein the medical device comprises about 10 μg to about 10 mg of the anti-scarring agent.

7952. The method of item 7742 wherein the medical device comprises about 10 mg to about 250 mg of the anti-scarring agent.

7953. The method of item 7742 wherein the medical device comprises about 250 mg to about 1000 mg of the anti-scarring agent.

7954. The method of item 7742 wherein the medical device comprises about 1000 mg to about 2500 mg of the anti-scarring agent.

7955. The method of item 7742 wherein a surface of the medical device comprises less than 0.01 μg of the anti-scarring agent per mm² of medical device surface to which the anti-scarring agent is applied.

7956. The method of item 7742 wherein a surface of the medical device comprises about 0.01 μg to about 1 μg of the anti-scarring agent per mm² of medical device surface to which the anti-scarring agent is applied.

7957. The method of item 7742 wherein a surface of the medical device comprises about 1 μg to about 10 μg of the anti-scarring agent per mm² of medical device surface to which the anti-scarring agent is applied.

7958. The method of item 7742 wherein a surface of the medical device comprises about 10 μg to about 250 μg of the anti-scarring agent per mm² of medical device surface to which the anti-scarring agent is applied.

7959. The method of item 7742 wherein a surface of the medical device comprises about 250 μg to about 1000 μg of the anti-scarring agent of anti-scarring agent per mm² of medical device surface to which the anti-scarring agent is applied.

7960. The method of item 7742 wherein a surface of the medical device comprises about 1000 μg to about 2500 μg of the anti-scarring agent per mm² of medical device surface to which the anti-scarring agent is applied.

7961. The method of item 7742 wherein the combining is performed by direct affixing the agent or the composition to the electrical device.

7962. The method of item 7742 wherein the combining is performed by spraying the agent or the component onto the electrical device.

7963. The method of item 7742 wherein the combining is performed by electrospraying the agent or the composition onto the electrical device.

7964. The method of item 7742 wherein the combining is performed by dipping the electrical device into a solution comprising the agent or the composition.

7965. The method of item 7742 wherein the combining is performed by covalently attaching the agent or the composition to the electrical device.

7966. The method of item 7742 wherein the combining is performed by non-covalently attaching the agent or the composition to the electrical device.

7967. The method of item 7742 wherein the combining is performed by coating the electrical device with a substance that contains the agent or the composition.

7968. The method of item 7742 wherein the combining is performed by coating the electrical device with a substance that absorbs the agent.

7969. The method of item 7742 wherein the combining is performed by interweaving the electrical device with a thread composed of, or coated with, the agent or the composition.

7970. The method of item 7742 wherein the combining is performed by completely covering the electrical device with a sleeve that contains the agent or the composition.

7971. The method of item 7742 wherein the combining is performed by covering a portion of the electrical device with a sleeve that contains the agent or the composition.

7972. The method of item 7742 wherein the combining is performed by completely covering the electrical device with a cover that contains the agent or the composition.

7973. The method of item 7742 wherein the combining is performed by covering a portion of the electrical device with a cover that contains the agent or the composition.

7974. The method of item 7742 wherein the combining is performed by completely covering the electrical device with an electrospun fabric that contains the agent or the composition.

7975. The method of item 7742 wherein the combining is performed by covering a portion of the electrical device with an electrospun fabric that contains the agent or the composition.

7976. The method of item 7742 wherein the combining is performed by completely covering the electrical device with a mesh that contains the agent or the composition.

7977. The method of item 7742 wherein the combining is performed by covering a portion of the electrical device with a mesh that contains the agent or the composition.

7978. The method of item 7742 wherein the combining is performed by constructing a portion of the electrical device with the agent or the composition.

7979. The method of item 7742 wherein the combining is performed by impregnating the electrical device with the agent or the composition.

7980. The method of item 7742 wherein the combining is performed by constructing a portion of the electrical device from a degradable polymer that releases the agent.

7981. The method of item 7742 wherein the combining is performed by dipping the electrical device into a solution that comprise the agent and an inert solvent for the electrical device.

7982. The method of item 7742 wherein the combining is performed by dipping the electrical device into a solution that comprises the agent and a solvent that will swell the electrical device.

7983. The method of item 7742 wherein the combining is performed by dipping the electrical device into a solution that comprises the agent and a solvent that will dissolve the electrical device.

7984. The method of item 7742 wherein the combining is performed by dipping the electrical device into a solution that comprises the agent, a polymer and an inert solvent for the electrical device.

7985. The method of item 7742 wherein the combining is performed by dipping the electrical device into a solution that comprises the agent, a polymer and a solvent that will swell the electrical device.

7986. The method of item 7742 wherein the combining is performed by dipping the electrical device into a solution that comprises the agent, a polymer and a solvent that will dissolve the electrical device.

7987. The method of item 7742 wherein the combining is performed by spraying the electrical device into a solution that comprises the agent and an inert solvent for the electrical device.

7988. The method of item 7742 wherein the combining is performed by spraying the electrical device into a solution that comprises the agent and a solvent that will swell the electrical device.

7989. The method of item 7742 wherein the combining is performed by spraying the electrical device into a solution that comprises the agent and a solvent that will dissolve the electrical device.

7990. The method of item 7742 wherein the combining is performed by spraying the electrical device into a solution that comprises the agent, a polymer and an inert solvent for the electrical device.

7991. The method of item 7742 wherein the combining is performed by spraying the electrical device into a solution that comprises the agent, a polymer and a solvent that will swell the electrical device.

7992. The method of item 7742 wherein the combining is performed by spraying the electrical device into a solution that comprises the agent, a polymer and a solvent that will dissolve the electrical device.

7993. The method for making a medical device of items 7742-7992 wherein the chronic pain results from injury.

7994. The method for making a medical device of items 7742-7992 wherein the chronic pain results from an illness.

7995. The method for making a medical device of items 7742-7992 wherein the chronic pain results from scoliosis.

7996. The method for making a medical device of items 7742-7992 wherein the chronic pain results from spinal disc degeneration.

7997. The method for making a medical device of items 7742-7992 wherein the chronic pain results from malignancy.

7998. The method for making a medical device of items 7742-7992 wherein the chronic pain results from arachnoiditis.

7999. The method for making a medical device of items 7742-7992 wherein the chronic pain results from a chronic disease.

8000. The method for making a medical device of items 7742-7992 wherein the chronic pain results from a pain syndrome.

8001. The method for making a medical device of items 7742-7992 wherein the neurostimulator comprises a lead that delivers electrical stimulation to a nerve and an electrical connection that connects a power source to the lead.

8002. The method for making a medical device of items 7742-7992 wherein the neurostimulator is adapted for spinal cord stimulation, and comprises a sensor that detects the position of the spine and a stimulator that emits pulses that decrease in amplitude when the back is in a supine position.

8003. The method for making a medical device of items 7742-7992 wherein the neurostimulator comprises an electrode and a control circuit that generates pulses and rest period based on intervals corresponding to the host body's activity and regeneration period.

8004. The method for making a medical device of items 7742-7992 wherein the neurostimulator comprises a stimulation catheter lead and an electrode

8005. The method for making a medical device of items 7742-7992 wherein the neurostimulator is a self-centering epidural spinal cord lead.

8006. A method for making a medical device comprising: combining a neurostimulator for treating Parkinson's Disease (i.e., an electrical device) and an anti-scarring agent or a composition comprising an anti-scarring agent, wherein the agent inhibits scarring between the device and a host into which the device is implanted. C

8007. The method of item 8006 wherein the agent is an adensosine A2A receptor antagonist.

8008. The method of item 8006 wherein the agent is an AKT inhibitor.

8009. The method of item 8006 wherein the agent is an alpha 2 integrin antagonist, wherein the alpha 2 integrin antagonist is Pharmaprojects No. 5754 (Merck KgaA).

8010. The method of item 8006 wherein the agent is an alpha 4 integrin antagonist.

8011. The method of item 8006 wherein the agent is an alpha 7 nicotinic receptor agonist.

8012. The method of item 8006 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

8013. The method of item 8006 wherein the agent is an apoptosis antagonist.

8014. The method of item 8006 wherein the agent is an apoptosis activator.

8015. The method of item 8006 wherein the agent is a beta 1 integrin antagonist.

8016. The method of item 8006 wherein the agent is a beta tubulin inhibitor.

8017. The method of item 8006 wherein the agent is a blocker of enzyme production in Hepatitis C.

8018. The method of item 8006 wherein the agent is a Bruton's tyrosine kinase inhibitor.

8019. The method of item 8006 wherein the agent is a calcineurin inhibitor.

8020. The method of item 8006 wherein the agent is a caspase 3 inhibitor.

8021. The method of item 8006 wherein the agent is a CC chemokine receptor antagonist.

8022. The method of item 8006 wherein the agent is a cell cycle inhibitor selected from the group consisting of SNS-595 (Sunesis), synthadotin, KRX-0403, and an analogue or derivative thereof.

8023. The method of item 8006 wherein the agent is a cathepsin B inhibitor.

8024. The method of item 8006 wherein the agent is a cathepsin K inhibitor, wherein the cathepsin K inhibitor is 462795 (GlaxoSmithKline), INPL-022-D6 (Amura Therapeutics), or an analogue or derivative thereof.

8025. The method of item 8006 wherein the agent is a cathepsin L inhibitor.

8026. The method of item 8006 wherein the agent is a CD40 antagonist.

8027. The method of item 8006 wherein the agent is a chemokine receptor agonist.

8028. The method of item 8006 wherein the agent is a chymase inhibitor.

8029. The method of item 8006 wherein the agent is a collagenase antagonist.

8030. The method of item 8006 wherein the agent is a CXCR antagonist.

8031. The method of item 8006 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAKI inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

8032. The method of item 8006 wherein the agent is a cyclooxygenase 1 inhibitor

8033. The method of item 8006 wherein the agent is a DHFR inhibitor.

8034. The method of item 8006 wherein the agent is a dual integrin inhibitor.

8035. The method of item 8006 wherein the agent is an elastase inhibitor.

8036. The method of item 8006 wherein the agent is an elongation factor-1 alpha inhibitor.

8037. The method of item 8006 wherein the agent is an endothelial growth factor antagonist.

8038. The method of item 8006 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

8039. The method of item 8006 wherein the agent is an endotoxin antagonist.

8040. The method of item 8006 wherein the agent is an epothilone and tubulin binder.

8041. The method of item 8006 wherein the agent is an estrogen receptor antagonist.

8042. The method of item 8006 wherein the agent is an FGF inhibitor.

8043. The method of item 8006 wherein the agent is a farnexyl transferase inhibitor.

8044. The method of item 8006 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

8045. The method of item 8006 wherein the agent is an FLT-3 kinase inhibitor.

8046. The method of item 8006 wherein the agent is an FGF receptor kinase inhibitor.

8047. The method of item 8006 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

8048. The method of item 8006 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

8049. The method of item 8006 wherein the agent is a histone deacetylase inhibitor.

8050. The method of item 8006 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

8051. The method of item 8006 wherein the agent is an ICAM inhibitor.

8052. The method of item 8006 wherein the agent is an IL, ICE and IRAK antagonist, wherein the antagonist is a CJ-14877, CP-424174 (Pfizer), NF-61 (Negma-Lerads), and an analogue or derivative thereof.

8053. The method of item 8006 wherein the agent is an IL-2 inhibitor.

8054. The method of item 8006 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

8055. The method of item 8006 wherein the agent is an IMPDH (inosine monophosphate).

8056. The method of item 8006 wherein the agent is an integrin antagonist.

8057. The method of item 8006 wherein the agent is an interleukin antagonist.

8058. The method of item 8006 wherein the agent is an inhibitor of type III receptor tyrosine kinase.

8059. The method of item 8006 wherein the agent is an irreversible inhibitor of enzyme methionine aminopeptidase type 2.

8060. The method of item 8006 wherein the agent is an isozyme selective delta protein kinase C inhibitor.

8061. The method of item 8006 wherein the agent a JAK3 enzyme inhibitor.

8062. The method of item 8006 wherein the agent is a JNK inhibitor.

8063. The method of item 8006 wherein the agent is a kinase inhibitor.

8064. The method of item 8006 wherein the agent is kinesin antagonist.

8065. The method of item 8006 wherein the agent is a kinesin antagonist.

8066. The method of item 8006 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

8067. The method of item 8006 wherein the agent is an MAP kinase inhibitor.

8068. The method of item 8006 wherein the agent is a matrix metalloproteinase inhibitor.

8069. The method of item 8006 wherein the agent is an MCP-CCR2 inhibitor.

8070. The method of item 8006 wherein the agent is an mTOR inhibitor.

8071. The method of item 8006 wherein the agent is an mTOR kinase inhibitor.

8072. The method of item 8006 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

8073. The method of item 8006 wherein the agent is an MIF inhibitor.

8074. The method of item 8006 wherein the agent is an MMP inhibitor.

8075. The method of item 8006 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

8076. The method of item 8006 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

8077. The method of item 8006 wherein the agent is a nitric oxide agonist.

8078. The method of item 8006 wherein the agent is an ornithine decarboxylase inhibitor.

8079. The method of item 8006 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

8080. The method of item 8006 wherein the agent is a palmitoyl-protein thioesterase inhibitor.

8081. The method of item 8006 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

8082. The method of item 8006 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

8083. The method of item 8006 wherein the agent is a phosphatase inhibitor.

8084. The method of item 8006 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

8085. The method of item 8006 wherein the agent is a PKC inhibitor.

8086. The method of item 8006 wherein the agent is a platelet activating factor antagonist.

8087. The method of item 8006 wherein the agent is a platelet-derived growth factor receptor kinase inhibitor.

8088. The method of item 8006 wherein the agent is a prolyl hydroxylase inhibitor.

8089. The method of item 8006 wherein the agent is a polymorphonuclear neutrophil inhibitor.

8090. The method of item 8006 wherein the agent is a protein kinase B inhibitor.

8091. The method of item 8006 wherein the agent is a protein kinase C stimulant.

8092. The method of item 8006 wherein the agent is a purine nucleoside analogue.

8093. The method of item 8006 wherein the agent is a purinoreceptor P2X antagonist.

8094. The method of item 8006 wherein the agent is a Raf kinase inhibitor.

8095. The method of item 8006 wherein the agent is a reversible inhibitor of ErbB1 and ErbB2.

8096. The method of item 8006 wherein the agent is a ribonucleoside triphosphate reductase inhibitor.

8097. The method of item 8006 wherein the agent is an SDF-1 antagonist.

8098. The method of item 8006 wherein the agent is a sheddase inhibitor.

8099. The method of item 8006 wherein the agent is an SRC inhibitor.

8100. The method of item 8006 wherein the agent is a stromelysin inhibitor.

8101. The method of item 8006 wherein the agent is an Syk kinase inhibitor.

8102. The method of item 8006 wherein the agent is a telomerase inhibitor.

8103. The method of item 8006 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

8104. The method of item 8006 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

8105. The method of item 8006 wherein the agent is a Toll receptor inhibitor.

8106. The method of item 8006 wherein the agent is a tubulin antagonist.

8107. The method of item 8006 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

8108. The method of item 8006 wherein the agent is a VEGF inhibitor.

8109. The method of item 8006 wherein the agent is a vitamin D receptor agonist.

8110. The method of item 8006 wherein the agent is ZD-6474 (an angiogenesis inhibitor).

8111. The method of item 8006 wherein the agent is AP-23573 (an mTOR inhibitor).

8112. The method of item 8006 wherein the agent is synthadotin (a tubulin antagonist).

8113. The method of item 8006 wherein the agent is S-0885 (a collagenase inhibitor).

8114. The method of item 8006 wherein the agent is aplidine (an elongation factor-1 alpha inhibitor).

8115. The method of item 8006 wherein the agent is ixabepilone (an epithilone).

8116. The method of item 8006 wherein the agent is IDN-5390 (an angiogenesis inhibitor).

8117. The method of item 8006 wherein the agent is SB-2723005 (an angiogenesis inhibitor).

8118. The method of item 8006 wherein the agent is ABT-518 (an angiogenesis inhibitor).

8119. The method of item 8006 wherein the agent is combretastatin (an angiogenesis inhibitor).

8120. The method of item 8006 wherein the agent is anecortave acetate (an angiogenesis inhibitor).

8121. The method of item 8006 wherein the agent is SB-715992 (a kinesin antagonist).

8122. The method of item 8006 wherein the agent is temsirolimus (an mTOR inhibitor).

8123. The method of item 8006 wherein the agent is adalimumab (a TNFα antagonist).

8124. The method of item 8006, wherein the composition comprises a polymer.

8125. The method of item 8006, wherein the composition comprises a polymeric carrier.

8126. The method of item 8006 wherein the anti-scarring agent inhibits adhesion between the medical device and a host into which the medical device is implanted.

8127. The method of item 8006 wherein the medical device delivers the anti-scarring agent locally to tissue proximate to the medical device.

8128. The method of item 8006 wherein the medical device has a coating that comprises the anti-scarring agent.

8129. The method of item 8006, wherein the medical device has a coating that comprises the agent and is disposed on a surface of the electrical device.

8130. The method of item 8006, wherein the medical device has a coating that comprises the agent and directly contacts the electrical device.

8131. The method of item 8006, wherein the medical device has a coating that comprises the agent and indirectly contacts the electrical device.

8132. The method of item 8006, wherein the medical device has a coating that comprises the agent and partially covers the electrical device.

8133. The method of item 8006, wherein the medical device has a coating that comprises the agent and completely covers the electrical device.

8134. The method of item 8006, wherein the medical device has a uniform coating.

8135. The method of item 8006, wherein the medical device has a non-uniform coating.

8136. The method of item 8006, wherein the medical device has a discontinuous coating.

8137. The method of item 8006, wherein the medical device has a patterned coating.

8138. The method of item 8006, wherein the medical device has a coating with a thickness of 100 μm or less.

8139. The method of item 8006, wherein the medical device has a coating with a thickness of 10 μm or less.

8140. The method of item 8006, wherein the medical device has a coating, and the coating adheres to the surface of the electrical device upon deployment of the electrical device.

8141. The method of item 8006, wherein the medical device has a coating, and wherein the coating is stable at room temperature for a period of 1 year.

8142. The method of item 8006, wherein the medical device has a coating, and wherein the anti-scarring agent is present in the coating in an amount ranging between about 0.0001% to about 1% by weight.

8143. The method of item 8006, wherein the medical device has a coating, and wherein the anti-scarring agent is present in the coating in an amount ranging between about 1% to about 10% by weight.

8144. The method of item 8006, wherein the medical device has a coating, and wherein the anti-scarring agent is present in the coating in an amount ranging between about 10% to about 25% by weight.

8145. The method of item 8006, wherein the medical device has a coating, and wherein the anti-scarring agent is present in the coating in an amount ranging between about 25% to about 70% by weight.

8146. The method of item 8006, wherein the medical device has a coating, and wherein the coating further comprises a polymer.

8147. The method of item 8006, wherein the medical device has a first coating having a first composition and a second coating having a second composition.

8148. The method of item 8006, wherein the medical device has a first coating having a first composition and a second coating having a second composition, wherein the first composition and the second composition are different.

8149. The method of item 8006, wherein the composition comprises a polymer.

8150. The method of item 8006, wherein the composition comprises a polymeric carrier.

8151. The method of item 8006, wherein the composition comprises a polymeric carrier, and wherein the polymeric carrier comprises a copolymer.

8152. The method of item 8006, wherein the composition comprises a polymeric carrier, and wherein the polymeric carrier comprises a block copolymer.

8153. The method of item 8006, wherein the composition comprises a polymeric carrier, and wherein the polymeric carrier comprises a random copolymer.

8154. The method of item 8006, wherein the composition comprises a polymeric carrier, and wherein the polymeric carrier comprises a biodegradable polymer.

8155. The method of item 8006, wherein the composition comprises a polymeric carrier, and wherein the polymeric carrier comprises a non-biodegradable polymer.

8156. The method of item 8006, wherein the composition comprises a polymeric carrier, and wherein the polymeric carrier comprises a hydrophilic polymer.

8157. The method of item 8006, wherein the composition comprises a polymeric carrier, and wherein the polymeric carrier comprises a hydrophobic polymer.

8158. The method of item 8006, wherein the composition comprises a polymeric carrier, and wherein the polymeric carrier comprises a polymer having hydrophilic domains.

8159. The method of item 8006, wherein the composition comprises a polymeric carrier, and wherein the polymeric carrier comprises a polymer having hydrophobic domains.

8160. The method of item 8006, wherein the composition comprises a polymeric carrier, and wherein the polymeric carrier comprises a non-conductive polymer.

8161. The method of item 8006, wherein the composition comprises a polymeric carrier, and wherein the polymeric carrier comprises an elastomer.

8162. The method of item 8006, wherein the composition comprises a polymeric carrier, and wherein the polymeric carrier comprises a hydrogel.

8163. The method of item 8006, wherein the composition comprises a polymeric carrier, and wherein the polymeric carrier comprises a silicone polymer.

8164. The method of item 8006, wherein the composition comprises a polymeric carrier, and wherein the polymeric carrier comprises a hydrocarbon polymer.

8165. The method of item 8006, wherein the composition comprises a polymeric carrier, and wherein the polymeric carrier comprises a styrene-derived polymer.

8166. The method of item 8006, wherein the composition comprises a polymeric carrier, and wherein the polymeric carrier comprises a butadiene polymer.

8167. The method of item 8006, wherein the composition comprises a polymeric carrier, and wherein the polymeric carrier comprises a macromer.

8168. The method of item 8006, wherein the composition comprises a polymeric carrier, and wherein the polymeric carrier comprises a poly(ethylene glycol) polymer.

8169. The method of item 8006 wherein the composition comprises a polymeric carrier, and wherein the polymeric carrier comprises an amorphous polymer.

8170. The method of item 8006, wherein the medical device comprises a lubricious coating.

8171. The method of item 8006 wherein the anti-scarring agent is located within pores or holes of the medical device.

8172. The method of item 8006 wherein the anti-scarring agent is located within a channel, lumen, or divet of the medical device.

8173. The method of item 8006, wherein the medical device further comprises a second pharmaceutically active agent.

8174. The method of item 8006 wherein the medical device further comprises an anti-inflammatory agent.

8175. The method of item 8006 wherein the medical device further comprises an agent that inhibits infection.

8176. The method of item 8006 wherein the medical device further comprises an agent that inhibits infection, and wherein the agent is an anthracycline.

8177. The method of item 8006 wherein the medical device further comprises an agent that inhibits infection, and wherein the agent is doxorubicin.

8178. The method of item 8006 wherein the medical device further comprises an agent that inhibits infection, and wherein the agent is mitoxantrone.

8179. The method of item 8006 wherein the medical device further comprises an agent that inhibits infection, and wherein the agent is a fluoropyrimidine.

8180. The method of item 8006 wherein the medical device further comprises an agent that inhibits infection, and wherein the agent is 5-fluorouracil (5-FU).

8181. The method of item 8006 wherein the medical device further comprises an agent that inhibits infection, and wherein the agent is a folic acid antagonist.

8182. The method of item 8006 wherein the medical device further comprises an agent that inhibits infection, and wherein the agent is methotrexate.

8183. The method of item 8006 wherein the medical device further comprises an agent that inhibits infection, and wherein the agent is a podophylotoxin.

8184. The method of item 8006 wherein the medical device further comprises an agent that inhibits infection, and wherein the agent is etoposide

8185. The method of item 8006 wherein the medical device further comprises an agent that inhibits infection, and wherein the agent is a camptothecin.

8186. The method of item 8006 wherein the medical device further comprises an agent that inhibits infection, and wherein the agent is a hydroxyurea.

8187. The method of item 8006 wherein the medical device further comprises an agent that inhibits infection, and wherein the agent is a platinum complex.

8188. The method of item 8006 wherein the medical device further comprises an agent that inhibits infection, and wherein the agent is cisplatin.

8189. The method of item 8006 wherein the medical device further comprises an anti-thrombotic agent.

8190. The method of item 8006 wherein the medical device further comprises a visualization agent.

8191. The method of item 8006 wherein the medical device further comprises a visualization agent, wherein the visualization agent is a radiopaque material, and wherein the radiopaque material further comprises a metal, a halogenated compound, or a barium containing compound.

8192. The method of item 8006 wherein the medical device further comprises a visualization agent, wherein the visualization agent is a radiopaque material, and wherein the radiopaque material further comprises barium, tantalum, or technetium.

8193. The method of item 8006 wherein the medical device further comprises a visualization agent, and wherein the visualization agent is a MRI responsive material.

8194. The method of item 8006 wherein the medical device further comprises a visualization agent, and wherein the visualization agent further comprises a gadolinium chelate.

8195. The method of item 8006 wherein the medical device further comprises a visualization agent, and wherein the visualization agent further comprises iron, magnesium, manganese, copper, or chromium.

8196. The method of item 8006 wherein the medical device further comprises a visualization agent, and wherein the visualization agent further comprises an iron oxide compound.

8197. The method of item 8006 wherein the medical device further comprises a visualization agent, and wherein the visualization agent further comprises a dye, pigment, or colorant.

8198. The method of item 8006 wherein the medical device further comprises an echogenic material.

8199. The method of item 8006 wherein the medical device further comprises an echogenic material, and wherein the echogenic material is in the form of a coating.

8200. The method of item 8006 wherein the medical device is sterile.

8201. The method of item 8006 wherein the anti-scarring agent is released into tissue in the vicinity of the medical device after deployment of the medical device.

8202. The method of item 8006 wherein the anti-scarring agent is released into tissue in the vicinity of the medical device after deployment of the medical device, and wherein the tissue is connective tissue.

8203. The method of item 8006 wherein the anti-scarring agent is released into tissue in the vicinity of the medical device after deployment of the medical device, and wherein the tissue is muscle tissue.

8204. The method of item 8006 wherein the anti-scarring agent is released into tissue in the vicinity of the medical device after deployment of the medical device, and wherein the tissue is nerve tissue.

8205. The method of item 8006 wherein the anti-scarring agent is released into tissue in the vicinity of the medical device after deployment of the medical device, and wherein the tissue is epithelium tissue.

8206. The method of item 8006 wherein the anti-scarring agent is released in effective concentrations from the medical device over a period ranging from the time of deployment of the medical device to about 1 year.

8207. The method of item 8006 wherein the anti-scarring agent is released in effective concentrations from the medical device over a period ranging from about 1 month to 6 months.

8208. The method of item 8006 wherein the anti-scarring agent is released in effective concentrations from the medical device over a period ranging from about 1-90 days.

8209. The method of item 8006 wherein the anti-scarring agent is released in effective concentrations from the medical device at a constant rate.

8210. The method of item 8006 wherein the anti-scarring agent is released in effective concentrations from the medical device at an increasing rate.

8211. The method of item 8006 wherein the anti-scarring agent is released in effective concentrations from the medical device at a decreasing rate.

8212. The method of item 8006 wherein the anti-scarring agent is released in effective concentrations from the composition comprising the anti-scarring agent by diffusion over a period ranging from the time of deployment of the medical device to about 90 days.

8213. The method of item 8006 wherein the anti-scarring agent is released in effective concentrations from the composition comprising the anti-scarring agent by erosion of the composition over a period ranging from the time of deployment of the medical device to about 90 days.

8214. The method of item 8006 wherein the medical device comprises about 0.01 μg to about 10 μg of the anti-scarring agent.

8215. The method of item 8006 wherein the medical device comprises about 10 μg to about 10 mg of the anti-scarring agent.

8216. The method of item 8006 wherein the medical device comprises about 10 mg to about 250 mg of the anti-scarring agent.

8217. The method of item 8006 wherein the medical device comprises about 250 mg to about 1000 mg of the anti-scarring agent.

8218. The method of item 8006 wherein the medical device comprises about 1000 mg to about 2500 mg of the anti-scarring agent.

8219. The method of item 8006 wherein a surface of the medical device comprises less than 0.01 μg of the anti-scarring agent per mm² of medical device surface to which the anti-scarring agent is applied.

8220. The method of item 8006 wherein a surface of the medical device comprises about 0.01 μg to about 1 μg of the anti-scarring agent per mm² of medical device surface to which the anti-scarring agent is applied.

8221. The method of item 8006 wherein a surface of the medical device comprises about 1 μg to about 10 μg of the anti-scarring agent per mm² of medical device surface to which the anti-scarring agent is applied.

8222. The method of item 8006 wherein a surface of the medical device comprises about 10 μg to about 250 μg of the anti-scarring agent per mm² of medical device surface to which the anti-scarring agent is applied.

8223. The method of item 8006 wherein a surface of the medical device comprises about 250 μg to about 1000 μg of the anti-scarring agent of anti-scarring agent per mm² of medical device surface to which the anti-scarring agent is applied.

8224. The method of item 8006 wherein a surface of the medical device comprises about 1000 μg to about 2500 μg of the anti-scarring agent per mm² of medical device surface to which the anti-scarring agent is applied.

8225. The method of item 8006 wherein the combining is performed by direct affixing the agent or the composition to the electrical device.

8226. The method of item 8006 wherein the combining is performed by spraying the agent or the component onto the electrical device.

8227. The method of item 8006 wherein the combining is performed by electrospraying the agent or the composition onto the electrical device.

8228. The method of item 8006 wherein the combining is performed by dipping the electrical device into a solution comprising the agent or the composition.

8229. The method of item 8006 wherein the combining is performed by covalently attaching the agent or the composition to the electrical device.

8230. The method of item 8006 wherein the combining is performed by non-covalently attaching the agent or the composition to the electrical device.

8231. The method of item 8006 wherein the combining is performed by coating the electrical device with a substance that contains the agent or the composition.

8232. The method of item 8006 wherein the combining is performed by coating the electrical device with a substance that absorbs the agent.

8233. The method of item 8006 wherein the combining is performed by interweaving the electrical device with a thread composed of, or coated with, the agent or the composition.

8234. The method of item 8006 wherein the combining is performed by completely covering the electrical device with a sleeve that contains the agent or the composition.

8235. The method of item 8006 wherein the combining is performed by covering a portion of the electrical device with a sleeve that contains the agent or the composition.

8236. The method of item 8006 wherein the combining is performed by completely covering the electrical device with a cover that contains the agent or the composition.

8237. The method of item 8006 wherein the combining is performed by covering a portion of the electrical device with a cover that contains the agent or the composition.

8238. The method of item 8006 wherein the combining is performed by completely covering the electrical device with an electrospun fabric that contains the agent or the composition.

8239. The method of item 8006 wherein the combining is performed by covering a portion of the electrical device with an electrospun fabric that contains the agent or the composition.

8240. The method of item 8006 wherein the combining is performed by completely covering the electrical device with a mesh that contains the agent or the composition.

8241. The method of item 8006 wherein the combining is performed by covering a portion of the electrical device with a mesh that contains the agent or the composition.

8242. The method of item 8006 wherein the combining is performed by constructing a portion of the electrical device with the agent or the composition.

8243. The method of item 8006 wherein the combining is performed by impregnating the electrical device with the agent or the composition.

8244. The method of item 8006 wherein the combining is performed by constructing a portion of the electrical device from a degradable polymer that releases the agent.

8245. The method of item 8006 wherein the combining is performed by dipping the electrical device into a solution that comprise the agent and an inert solvent for the electrical device.

8246. The method of item 8006 wherein the combining is performed by dipping the electrical device into a solution that comprises the agent and a solvent that will swell the electrical device.

8247. The method of item 8006 wherein the combining is performed by dipping the electrical device into a solution that comprises the agent and a solvent that will dissolve the electrical device.

8248. The method of item 8006 wherein the combining is performed by dipping the electrical device into a solution that comprises the agent, a polymer and an inert solvent for the electrical device.

8249. The method of item 8006 wherein the combining is performed by dipping the electrical device into a solution that comprises the agent, a polymer and a solvent that will swell the electrical device

8250. The method of item 8006 wherein the combining is performed by dipping the electrical device into a solution that comprises the agent, a polymer and a solvent that will dissolve the electrical device.

8251. The method of item 8006 wherein the combining is performed by spraying the electrical device into a solution that comprises the agent and an inert solvent for the electrical device.

8252. The method of item 8006 wherein the combining is performed by spraying the electrical device into a solution that comprises the agent and a solvent that will swell the electrical device.

8253. The method of item 8006 wherein the combining is performed by spraying the electrical device into a solution that comprises the agent and a solvent that will dissolve the electrical device.

8254. The method of item 8006 wherein the combining is performed by spraying the electrical device into a solution that comprises the agent, a polymer and an inert solvent for the electrical device.

8255. The method of item 8006 wherein the combining is performed by spraying the electrical device into a solution that comprises the agent, a polymer and a solvent that will swell the electrical device.

8256. The method of item 8006 wherein the combining is performed by spraying the electrical device into a solution that comprises the agent, a polymer and a solvent that will dissolve the electrical device.

8257. The method for making a medical device of any one of items 8006-8256 wherein the neurostimulator comprises an intracranially implantable electrical control module and an electrode.

8258. The method for making a medical device of any one of items 8006-8256 wherein the neurostimulator comprises a sensor and an electrode.

8259. A method for making a medical device comprising: combining a vagal nerve stimulator for treating epilepsy (i.e., an electrical device) and an anti-scarring agent or a composition comprising an anti-scarring agent, wherein the agent inhibits scarring between the device and a host into which the device is implanted.

8260. The method of item 8259 wherein the agent is an adensosine A2A receptor antagonist.

8261. The method of item 8259 wherein the agent is an AKT inhibitor.

8262. The method of item 8259 wherein the agent is an alpha 2 integrin antagonist, wherein the alpha 2 integrin antagonist is Pharmaprojects No. 5754 (Merck KgaA).

8263. The method of item 8259 wherein the agent is an alpha 4 integrin antagonist.

8264. The method of item 8259 wherein the agent is an alpha 7 nicotinic receptor agonist.

8265. The method of item 8259 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

8266. The method of item 8259 wherein the agent is an apoptosis antagonist.

8267. The method of item 8259 wherein the agent is an apoptosis activator.

8268. The method of item 8259 wherein the agent is a beta 1 integrin antagonist.

8269. The method of item 8259 wherein the agent is a beta tubulin inhibitor.

8270. The method of item 8259 wherein the agent is a blocker of enzyme production in Hepatitis C.

8271. The method of item 8259 wherein the agent is a Bruton's tyrosine kinase inhibitor.

8272. The method of item 8259 wherein the agent is a calcineurin inhibitor.

8273. The method of item 8259 wherein the agent is a caspase 3 inhibitor.

8274. The method of item 8259 wherein the agent is a CC chemokine receptor antagonist.

8275. The method of item 8259 wherein the agent is a cell cycle inhibitor selected from the group consisting of SNS-595 (Sunesis), synthadotin, KRX-0403, and an analogue or derivative thereof.

8276. The method of item 8259 wherein the agent is a cathepsin B inhibitor.

8277. The method of item 8259 wherein the agent is a cathepsin K inhibitor, wherein the cathepsin K inhibitor is 462795 (GlaxoSmithKline), INPL-022-D6 (Amura Therapeutics), or an analogue or derivative thereof.

8278. The method of item 8259 wherein the agent is a cathepsin L inhibitor.

8279. The method of item 8259 wherein the agent is a CD40 antagonist.

8280. The method of item 8259 wherein the agent is a chemokine receptor agonist.

8281. The method of item 8259 wherein the agent is a chymase inhibitor.

8282. The method of item 8259 wherein the agent is a collagenase antagonist.

8283. The method of item 8259 wherein the agent is a CXCR antagonist.

8284. The method of item 8259 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

8285. The method of item 8259 wherein the agent is a cyclooxygenase 1 inhibitor.

8286. The method of item 8259 wherein the agent is a DHFR inhibitor.

8287. The method of item 8259 wherein the agent is a dual integrin inhibitor.

8288. The method of item 8259 wherein the agent is an elastase inhibitor.

8289. The method of item 8259 wherein the agent is an elongation factor-1 alpha inhibitor.

8290. The method of item 8259 wherein the agent is an endothelial growth factor antagonist.

8291. The method of item 8259 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

8292. The method of item 8259 wherein the agent is an endotoxin antagonist.

8293. The method of item 8259 wherein the agent is an epothilone and tubulin binder.

8294. The method of item 8259 wherein the agent is an estrogen receptor antagonist.

8295. The method of item 8259 wherein the agent is an FGF inhibitor.

8296. The method of item 8259 wherein the agent is a farnexyl transferase inhibitor.

8297. The method of item 8259 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

8298. The method of item 8259 wherein the agent is an FLT-3 kinase inhibitor.

8299. The method of item 8259 wherein the agent is an FGF receptor kinase inhibitor.

8300. The method of item 8259 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

8301. The method of item 8259 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

8302. The method of item 8259 wherein the agent is a histone deacetylase inhibitor.

8303. The method of item 8259 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

8304. The method of item 8259 wherein the agent is an ICAM inhibitor.

8305. The method of item 8259 wherein the agent is an IL, ICE and IRAK antagonist, wherein the antagonist is a CJ-14877, CP-424174 (Pfizer), NF-61 (Negma-Lerads), and an analogue or derivative thereof.

8306. The method of item 8259 wherein the agent is an IL-2 inhibitor.

8307. The method of item 8259 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

8308. The method of item 8259 wherein the agent is an IMPDH (inosine monophosphate).

8309. The method of item 8259 wherein the agent is an integrin antagonist.

8310. The method of item 8259 wherein the agent is an interleukin antagonist.

8311. The method of item 8259 wherein the agent is an inhibitor of type III receptor tyrosine kinase.

8312. The method of item 8259 wherein the agent is an irreversible inhibitor of enzyme methionine aminopeptidase type 2.

8313. The method of item 8259 wherein the agent is an isozyme selective delta protein kinase C inhibitor.

8314. The method of item 8259 wherein the agent a JAK3 enzyme inhibitor.

8315. The method of item 8259 wherein the agent is a JNK inhibitor.

8316. The method of item 8259 wherein the agent is a kinase inhibitor.

8317. The method of item 8259 wherein the agent is kinesin antagonist.

8318. The method of item 8259 wherein the agent is a kinesin antagonist.

8319. The method of item 8259 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

8320. The method of item 8259 wherein the agent is an MAP kinase inhibitor.

8321. The method of item 8259 wherein the agent is a matrix metalloproteinase inhibitor.

8322. The method of item 8259 wherein the agent is an MCP-CCR2 inhibitor.

8323. The method of item 8259 wherein the agent is an mTOR inhibitor.

8324. The method of item 8259 wherein the agent is an mTOR kinase inhibitor.

8325. The method of item 8259 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

8326. The method of item 8259 wherein the agent is an MIF inhibitor.

8327. The method of item 8259 wherein the agent is an MMP inhibitor.

8328. The method of item 8259 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

8329. The method of item 8259 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

8330. The method of item 8259 wherein the agent is a nitric oxide agonist.

8331. The method of item 8259 wherein the agent is an ornithine decarboxylase inhibitor.

8332. The method of item 8259 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

8333. The method of item 8259 wherein the agent is a palmitoyl-protein thioesterase inhibitor.

8334. The method of item 8259 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

8335. The method of item 8259 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MG-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

8336. The method of item 8259 wherein the agent is a phosphatase inhibitor.

8337. The method of item 8259 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

8338. The method of item 8259 wherein the agent is a PKC inhibitor.

8339. The method of item 8259 wherein the agent is a platelet activating factor antagonist.

8340. The method of item 8259 wherein the agent is a platelet-derived growth factor receptor kinase inhibitor.

8341. The method of item 8259 wherein the agent is a prolyl hydroxylase inhibitor.

8342. The method of item 8259 wherein the agent is a polymorphonuclear neutrophil inhibitor.

8343. The method of item 8259 wherein the agent is a protein kinase B inhibitor.

8344. The method of item 8259 wherein the agent is a protein kinase C stimulant.

8345. The method of item 8259 wherein the agent is a purine nucleoside analogue.

8346. The method of item 8259 wherein the agent is a purinoreceptor P2X antagonist.

8347. The method of item 8259 wherein the agent is a Raf kinase inhibitor.

8348. The method of item 8259 wherein the agent is a reversible inhibitor of ErbB1 and ErbB2.

8349. The method of item 8259 wherein the agent is a ribonucleoside triphosphate reductase inhibitor.

8350. The method of item 8259 wherein the agent is an SDF-1 antagonist.

8351. The method of item 8259 wherein the agent is a sheddase inhibitor.

8352. The method of item 8259 wherein the agent is an SRC inhibitor.

8353. The method of item 8259 wherein the agent is a stromelysin inhibitor.

8354. The method of item 8259 wherein the agent is an Syk kinase inhibitor.

8355. The method of item 8259 wherein the agent is a telomerase inhibitor.

8356. The method of item 8259 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

8357. The method of item 8259 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

8358. The method of item 8259 wherein the agent is a Toll receptor inhibitor.

8359. The method of item 8259 wherein the agent is a tubulin antagonist.

8360. The method of item 8259 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

8361. The method of item 8259 wherein the agent is a VEGF inhibitor.

8362. The method of item 8259 wherein the agent is a vitamin D receptor agonist.

8363. The method of item 8259 wherein the agent is ZD-6474 (an angiogenesis inhibitor).

8364. The method of item 8259 wherein the agent is AP-23573 (an mTOR inhibitor).

8365. The method of item 8259 wherein the agent is synthadotin (a tubulin antagonist).

8366. The method of item 8259 wherein the agent is S-0885 (a collagenase inhibitor).

8367. The method of item 8259 wherein the agent is aplidine (an elongation factor-1 alpha inhibitor).

8368. The method of item 8259 wherein the agent is ixabepilone (an epithilone).

8369. The method of item 8259 wherein the agent is IDN-5390 (an angiogenesis inhibitor).

8370. The method of item 8259 wherein the agent is SB-2723005 (an angiogenesis inhibitor).

8371. The method of item 8259 wherein the agent is ABT-518 (an angiogenesis inhibitor).

8372. The method of item 8259 wherein the agent is combretastatin (an angiogenesis inhibitor).

8373. The method of item 8259 wherein the agent is anecortave acetate (an angiogenesis inhibitor).

8374. The method of item 8259 wherein the agent is SB-715992 (a kinesin antagonist).

8375. The method of item 8259 wherein the agent is temsirolimus (an mTOR inhibitor).

8376. The method of item 8259 wherein the agent is adalimumab (a TNFα antagonist).

8377. The method of item 8259, wherein the composition comprises a polymer.

8378. The method of item 8259, wherein the composition comprises a polymeric carrier.

8379. The method of item 8259 wherein the anti-scarring agent inhibits adhesion between the medical device and a host into which the medical device is implanted.

8380. The method of item 8259 wherein the medical device delivers the anti-scarring agent locally to tissue proximate to the medical device.

8381. The method of item 8259 wherein the medical device has a coating that comprises the anti-scarring agent.

8382. The method of item 8259, wherein the medical device has a coating that comprises the agent and is disposed on a surface of the electrical device.

8383. The method of item 8259, wherein the medical device has a coating that comprises the agent and directly contacts the electrical device.

8384. The method of item 8259, wherein the medical device has a coating that comprises the agent and indirectly contacts the electrical device.

8385. The method of item 8259, wherein the medical device has a coating that comprises the agent and partially covers the electrical device.

8386. The method of item 8259, wherein the medical device has a coating that comprises the agent and completely covers the electrical device.

8387. The method of item 8259, wherein the medical device has a uniform coating.

8388. The method of item 8259, wherein the medical device has a non-uniform coating.

8389. The method of item 8259, wherein the medical device has a discontinuous coating.

8390. The method of item 8259, wherein the medical device has a patterned coating.

8391. The method of item 8259, wherein the medical device has a coating with a thickness of 100 μm or less.

8392. The method of item 8259, wherein the medical device has a coating with a thickness of 10 μm or less.

8393. The method of item 8259, wherein the medical device has a coating, and the coating adheres to the surface of the electrical device upon deployment of the electrical device.

8394. The method of item 8259, wherein the medical device has a coating, and wherein the coating is stable at room temperature for a period of 1 year.

8395. The method of item 8259, wherein the medical device has a coating, and wherein the anti-scarring agent is present in the coating in an amount ranging between about 0.0001% to about 1% by weight.

8396. The method of item 8259, wherein the medical device has a coating, and wherein the anti-scarring agent is present in the coating in an amount ranging between about 1% to about 10% by weight.

8397. The method of item 8259, wherein the medical device has a coating, and wherein the anti-scarring agent is present in the coating in an amount ranging between about 10% to about 25% by weight.

8398. The method of item 8259, wherein the medical device has a coating, and wherein the anti-scarring agent is present in the coating in an amount ranging between about 25% to about 70% by weight.

8399. The method of item 8259, wherein the medical device has a coating, and wherein the coating further comprises a polymer.

8400. The method of item 8259, wherein the medical device has a first coating having a first composition and a second coating having a second composition.

8401. The method of item 8259, wherein the medical device has a first coating having a first composition and a second coating having a second composition, wherein the first composition and the second composition are different.

8402. The method of item 8259, wherein the composition comprises a polymer.

8403. The method of item 8259, wherein the composition comprises a polymeric carrier.

8404. The method of item 8259, wherein the composition comprises a polymeric carrier, and wherein the polymeric carrier comprises a copolymer.

8405. The method of item 8259, wherein the composition comprises a polymeric carrier, and wherein the polymeric carrier comprises a block copolymer.

8406. The method of item 8259, wherein the composition comprises a polymeric carrier, and wherein the polymeric carrier comprises a random copolymer.

8407. The method of item 8259, wherein the composition comprises a polymeric carrier, and wherein the polymeric carrier comprises a biodegradable polymer.

8408. The method of item 8259, wherein the composition comprises a polymeric carrier, and wherein the polymeric carrier comprises a non-biodegradable polymer.

8409. The method of item 8259, wherein the composition comprises a polymeric carrier, and wherein the polymeric carrier comprises a hydrophilic polymer.

8410. The method of item 8259, wherein the composition comprises a polymeric carrier, and wherein the polymeric carrier comprises a hydrophobic polymer.

8411. The method of item 8259, wherein the composition comprises a polymeric carrier, and wherein the polymeric carrier comprises a polymer having hydrophilic domains.

8412. The method of item 8259, wherein the composition comprises a polymeric carrier, and wherein the polymeric carrier comprises a polymer having hydrophobic domains.

8413. The method of item 8259, wherein the composition comprises a polymeric carrier, and wherein the polymeric carrier comprises a non-conductive polymer.

8414. The method of item 8259, wherein the composition comprises a polymeric carrier, and wherein the polymeric carrier comprises an elastomer.

8415. The method of item 8259, wherein the composition comprises a polymeric carrier, and wherein the polymeric carrier comprises a hydrogel.

8416. The method of item 8259, wherein the composition comprises a polymeric carrier, and wherein the polymeric carrier comprises a silicone polymer.

8417. The method of item 8259, wherein the composition comprises a polymeric carrier, and wherein the polymeric carrier comprises a hydrocarbon polymer.

8418. The method of item 8259, wherein the composition comprises a polymeric carrier, and wherein the polymeric carrier comprises a styrene-derived polymer.

8419. The method of item 8259, wherein the composition comprises a polymeric carrier, and wherein the polymeric carrier comprises a butadiene polymer

8420. The method of item 8259, wherein the composition comprises a polymeric carrier, and wherein the polymeric carrier comprises a macromer.

8421. The method of item 8259, wherein the composition comprises a polymeric carrier, and wherein the polymeric carrier comprises a poly(ethylene glycol) polymer.

8422. The method of item 8259 wherein the composition comprises a polymeric carrier, and wherein the polymeric carrier comprises an amorphous polymer.

8423. The method of item 8259, wherein the medical device comprises a lubricious coating.

8424. The method of item 8259 wherein the anti-scarring agent is located within pores or holes of the medical device.

8425. The method of item 8259 wherein the anti-scarring agent is located within a channel, lumen, or divet of the medical device.

8426. The method of item 8259, wherein the medical device further comprises a second pharmaceutically active agent.

8427. The method of item 8259 wherein the medical device further comprises an anti-inflammatory agent.

8428. The method of item 8259 wherein the medical device further comprises an agent that inhibits infection.

8429. The method of item 8259 wherein the medical device further comprises an agent that inhibits infection, and wherein the agent is an anthracycline.

8430. The method of item 8259 wherein the medical device further comprises an agent that inhibits infection, and wherein the agent is doxorubicin

8431. The method of item 8259 wherein the medical device further comprises an agent that inhibits infection, and wherein the agent is mitoxantrone.

8432. The method of item 8259 wherein the medical device further comprises an agent that inhibits infection, and wherein the agent is a fluoropyrimidine.

8433. The method of item 8259 wherein the medical device further comprises an agent that inhibits infection, and wherein the agent is 5-fluorouracil (5-FU).

8434. The method of item 8259 wherein the medical device further comprises an agent that inhibits infection, and wherein the agent is a folic acid antagonist.

8435. The method of item 8259 wherein the medical device further comprises an agent that inhibits infection, and wherein the agent is methotrexate.

8436. The method of item 8259 wherein the medical device further comprises an agent that inhibits infection, and wherein the agent is a podophylotoxin.

8437. The method of item 8259 wherein the medical device further comprises an agent that inhibits infection, and wherein the agent is etoposide.

8438. The method of item 8259 wherein the medical device further comprises an agent that inhibits infection, and wherein the agent is a camptothecin.

8439. The method of item 8259 wherein the medical device further comprises an agent that inhibits infection, and wherein the agent is a hydroxyurea.

8440. The method of item 8259 wherein the medical device further comprises an agent that inhibits infection, and wherein the agent is a platinum complex.

8441. The method of item 8259 wherein the medical device further comprises an agent that inhibits infection, and wherein the agent is cisplatin.

8442. The method of item 8259 wherein the medical device further comprises an anti-thrombotic agent.

8443. The method of item 8259 wherein the medical device further comprises a visualization agent.

8444. The method of item 8259 wherein the medical device further comprises a visualization agent, wherein the visualization agent is a radiopaque material, and wherein the radiopaque material further comprises a metal, a halogenated compound, or a barium containing compound.

8445. The method of item 8259 wherein the medical device further comprises a visualization agent, wherein the visualization agent is a radiopaque material, and wherein the radiopaque material further comprises barium, tantalum, or technetium.

8446. The method of item 8259 wherein the medical device further comprises a visualization agent, and wherein the visualization agent is a MRI responsive material.

8447. The method of item 8259 wherein the medical device further comprises a visualization agent, and wherein the visualization agent further comprises a gadolinium chelate.

8448. The method of item 8259 wherein the medical device further comprises a visualization agent, and wherein the visualization agent further comprises iron, magnesium, manganese, copper, or chromium.

8449. The method of item 8259 wherein the medical device further comprises a visualization agent, and wherein the visualization agent further comprises an iron oxide compound.

8450. The method of item 8259 wherein the medical device further comprises a visualization agent, and wherein the visualization agent further comprises a dye, pigment, or colorant.

8451. The method of item 8259 wherein the medical device further comprises an echogenic material.

8452. The method of item 8259 wherein the medical device further comprises an echogenic material, and wherein the echogenic material is in the form of a coating

8453. The method of item 8259 wherein the medical device is sterile.

8454. The method of item 8259 wherein the anti-scarring agent is released into tissue in the vicinity of the medical device after deployment of the medical device.

8455. The method of item 8259 wherein the anti-scarring agent is released into tissue in the vicinity of the medical device after deployment of the medical device, and wherein the tissue is connective tissue.

8456. The method of item 8259 wherein the anti-scarring agent is released into tissue in the vicinity of the medical device after deployment of the medical device, and wherein the tissue is muscle tissue.

8457. The method of item 8259 wherein the anti-scarring agent is released into tissue in the vicinity of the medical device after deployment of the medical device, and wherein the tissue is nerve tissue.

8458. The method of item 8259 wherein the anti-scarring agent is released into tissue in the vicinity of the medical device after deployment of the medical device, and wherein the tissue is epithelium tissue

8459. The method of item 8259 wherein the anti-scarring agent is released in effective concentrations from the medical device over a period ranging from the time of deployment of the medical device to about 1 year.

8460. The method of item 8259 wherein the anti-scarring agent is released in effective concentrations from the medical device over a period ranging from about 1 month to 6 months.

8461. The method of item 8259 wherein the anti-scarring agent is released in effective concentrations from the medical device over a period ranging from about 1-90 days.

8462. The method of item 8259 wherein the anti-scarring agent is released in effective concentrations from the medical device at a constant rate.

8463. The method of item 8259 wherein the anti-scarring agent is released in effective concentrations from the medical device at an increasing rate.

8464. The method of item 8259 wherein the anti-scarring agent is released in effective concentrations from the medical device at a decreasing rate.

8465. The method of item 8259 wherein the anti-scarring agent is released in effective concentrations from the composition comprising the anti-scarring agent by diffusion over a period ranging from the time of deployment of the medical device to about 90 days.

8466. The method of item 8259 wherein the anti-scarring agent is released in effective concentrations from the composition comprising the anti-scarring agent by erosion of the composition over a period ranging from the time of deployment of the medical device to about 90 days.

8467. The method of item 8259 wherein the medical device comprises about 0.01 μg to about 10 μg of the anti-scarring agent.

8468. The method of item 8259 wherein the medical device comprises about 10 μg to about 10 mg of the anti-scarring agent.

8469. The method of item 8259 wherein the medical device comprises about 10 mg to about 250 mg of the anti-scarring agent.

8470. The method of item 8259 wherein the medical device comprises about 250 mg to about 1000 mg of the anti-scarring agent.

8471. The method of item 8259 wherein the medical device comprises about 1000 mg to about 2500 mg of the anti-scarring agent.

8472. The method of item 8259 wherein a surface of the medical device comprises less than 0.01 μg of the anti-scarring agent per mm² of medical device surface to which the anti-scarring agent is applied.

8473. The method of item 8259 wherein a surface of the medical device comprises about 0.01 μg to about 1 μg of the anti-scarring agent per mm² of medical device surface to which the anti-scarring agent is applied.

8474. The method of item 8259 wherein a surface of the medical device comprises about 1 μg to about 10 μg of the anti-scarring agent per mm² of medical device surface to which the anti-scarring agent is applied.

8475. The method of item 8259 wherein a surface of the medical device comprises about 10 μg to about 250 μg of the anti-scarring agent per mm² of medical device surface to which the anti-scarring agent is applied.

8476. The method of item 8259 wherein a surface of the medical device comprises about 250 μg to about 1000 μg of the anti-scarring agent of anti-scarring agent per mm² of medical device surface to which the anti-scarring agent is applied.

8477. The method of item 8259 wherein a surface of the medical device comprises about 1000 μg to about 2500 μg of the anti-scarring agent per mm² of medical device surface to which the anti-scarring agent is applied.

8478. The method of item 8259 wherein the combining is performed by direct affixing the agent or the composition to the electrical device.

8479. The method of item 8259 wherein the combining is performed by spraying the agent or the component onto the electrical device.

8480. The method of item 8259 wherein the combining is performed by electrospraying the agent or the composition onto the electrical device.

8481. The method of item 8259 wherein the combining is performed by dipping the electrical device into a solution comprising the agent or the composition.

8482. The method of item 8259 wherein the combining is performed by covalently attaching the agent or the composition to the electrical device.

8483. The method of item 8259 wherein the combining is performed by non-covalently attaching the agent or the composition to the electrical device.

8484. The method of item 8259 wherein the combining is performed by coating the electrical device with a substance that contains the agent or the composition.

8485. The method of item 8259 wherein the combining is performed by coating the electrical device with a substance that absorbs the agent.

8486. The method of item 8259 wherein the combining is performed by interweaving the electrical device with a thread composed of, or coated with, the agent or the composition.

8487. The method of item 8259 wherein the combining is performed by completely covering the electrical device with a sleeve that contains the agent or the composition.

8488. The method of item 8259 wherein the combining is performed by covering a portion of the electrical device with a sleeve that contains the agent or the composition.

8489. The method of item 8259 wherein the combining is performed by completely covering the electrical device with a cover that contains the agent or the composition.

8490. The method of item 8259 wherein the combining is performed by covering a portion of the electrical device with a cover that contains the agent or the composition.

8491. The method of item 8259 wherein the combining is performed by completely covering the electrical device with an electrospun fabric that contains the agent or the composition.

8492. The method of item 8259 wherein the combining is performed by covering a portion of the electrical device with an electrospun fabric that contains the agent or the composition.

8493. The method of item 8259 wherein the combining is performed by completely covering the electrical device with a mesh that contains the agent or the composition.

8494. The method of item 8259 wherein the combining is performed by covering a portion of the electrical device with a mesh that contains the agent or the composition.

8495. The method of item 8259 wherein the combining is performed by constructing a portion of the electrical device with the agent or the composition.

8496. The method of item 8259 wherein the combining is performed by impregnating the electrical device with the agent or the composition.

8497. The method of item 8259 wherein the combining is performed by constructing a portion of the electrical device from a degradable polymer that releases the agent.

8498. The method of item 8259 wherein the combining is performed by dipping the electrical device into a solution that comprise the agent and an inert solvent for the electrical device.

8499. The method of item 8259 wherein the combining is performed by dipping the electrical device into a solution that comprises the agent and a solvent that will swell the electrical device.

8500. The method of item 8259 wherein the combining is performed by dipping the electrical device into a solution that comprises the agent and a solvent that will dissolve the electrical device.

8501. The method of item 8259 wherein the combining is performed by dipping the electrical device into a solution that comprises the agent, a polymer and an inert solvent for the electrical device.

8502. The method of item 8259 wherein the combining is performed by dipping the electrical device into a solution that comprises the agent, a polymer and a solvent that will swell the electrical device.

8503. The method of item 8259 wherein the combining is performed by dipping the electrical device into a solution that comprises the agent, a polymer and a solvent that will dissolve the electrical device.

8504. The method of item 8259 wherein the combining is performed by spraying the electrical device into a solution that comprises the agent and an inert solvent for the electrical device.

8505. The method of item 8259 wherein the combining is performed by spraying the electrical device into a solution that comprises the agent and a solvent that will swell the electrical device.

8506. The method of item 8259 wherein the combining is performed by spraying the electrical device into a solution that comprises the agent and a solvent that will dissolve the electrical device.

8507. The method of item 8259 wherein the combining is performed by spraying the electrical device into a solution that comprises the agent, a polymer and an inert solvent for the electrical device.

8508. The method of item 8259 wherein the combining is performed by spraying the electrical device into a solution that comprises the agent, a polymer and a solvent that will swell the electrical device.

8509. The method of item 8259 wherein the combining is performed by spraying the electrical device into a solution that comprises the agent, a polymer and a solvent that will dissolve the electrical device.

8510. A method for making a medical device comprising: combining a vagal nerve stimulator (i.e., an electrical device) and an anti-scarring agent or a composition comprising an anti-scarring agent, wherein the agent inhibits scarring between the device and a host into which the device is implanted.

8511. The method of item 8510 wherein the agent is an adensosine A2A receptor antagonist.

8512. The method of item 8510 wherein the agent is an AKT inhibitor.

8513. The method of item 8510 wherein the agent is an alpha 2 integrin antagonist, wherein the alpha 2 integrin antagonist is Pharmaprojects No. 5754 (Merck KgaA).

8514. The method of item 8510 wherein the agent is an alpha 4 integrin antagonist.

8515. The method of item 8510 wherein the agent is an alpha 7 nicotinic receptor agonist.

8516. The method of item 8510 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

8517. The method of item 8510 wherein the agent is an apoptosis antagonist.

8518. The method of item 8510 wherein the agent is an apoptosis activator.

8519. The method of item 8510 wherein the agent is a beta 1 integrin antagonist.

8520. The method of item 8510 wherein the agent is a beta tubulin inhibitor.

8521. The method of item 8510 wherein the agent is a blocker of enzyme production in Hepatitis C.

8522. The method of item 8510 wherein the agent is a Bruton's tyrosine kinase inhibitor.

8523. The method of item 8510 wherein the agent is a calcineurin inhibitor.

8524. The method of item 8510 wherein the agent is a caspase 3 inhibitor

8525. The method of item 8510 wherein the agent is a CC chemokine receptor antagonist.

8526. The method of item 8510 wherein the agent is a cell cycle inhibitor selected from the group consisting of SNS-595 (Sunesis), synthadotin, KRX-0403, and an analogue or derivative thereof.

8527. The method of item 8510 wherein the agent is a cathepsin B inhibitor.

8528. The method of item 8510 wherein the agent is a cathepsin K inhibitor, wherein the cathepsin K inhibitor is 462795 (GlaxoSmithKline), INPL-022-D6 (Amura Therapeutics), or an analogue or derivative thereof.

8529. The method of item 8510 wherein the agent is a cathepsin L inhibitor.

8530. The method of item 8510 wherein the agent is a CD40 antagonist.

8531. The method of item 8510 wherein the agent is a chemokine receptor agonist.

8532. The method of item 8510 wherein the agent is a chymase inhibitor.

8533. The method of item 8510 wherein the agent is a collagenase antagonist.

8534. The method of item 8510 wherein the agent is a CXCR antagonist.

8535. The method of item 8510 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

8536. The method of item 8510 wherein the agent is a cyclooxygenase 1 inhibitor.

8537. The method of item 8510 wherein the agent is a DHFR inhibitor.

8538. The method of item 8510 wherein the agent is a dual integrin inhibitor.

8539. The method of item 8510 wherein the agent is an elastase inhibitor.

8540. The method of item 8510 wherein the agent is an elongation factor-1 alpha inhibitor.

8541. The method of item 8510 wherein the agent is an endothelial growth factor antagonist.

8542. The method of item 8510 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

8543. The method of item 8510 wherein the agent is an endotoxin antagonist.

8544. The method of item 8510 wherein the agent is an epothilone and tubulin binder.

8545. The method of item 8510 wherein the agent is an estrogen receptor antagonist.

8546. The method of item 8510 wherein the agent is an FGF inhibitor.

8547. The method of item 8510 wherein the agent is a farnexyl transferase inhibitor.

8548. The method of item 8510 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

8549. The method of item 8510 wherein the agent is an FLT-3 kinase inhibitor.

8550. The method of item 8510 wherein the agent is an FGF receptor kinase inhibitor.

8551. The method of item 8510 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

8552. The method of item 8510 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

8553. The method of item 8510 wherein the agent is a histone deacetylase inhibitor.

8554. The method of item 8510 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

8555. The method of item 8510 wherein the agent is an ICAM inhibitor.

8556. The method of item 8510 wherein the agent is an IL, ICE and IRAK antagonist, wherein the antagonist is a CJ-14877, CP-424174 (Pfizer), NF-61 (Negma-Lerads), and an analogue or derivative thereof.

8557. The method of item 8510 wherein the agent is an IL-2 inhibitor.

8558. The method of item 8510 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

8559. The method of item 8510 wherein the agent is an IMPDH (inosine monophosphate).

8560. The method of item 8510 wherein the agent is an integrin antagonist.

8561. The method of item 8510 wherein the agent is an interleukin antagonist.

8562. The method of item 8510 wherein the agent is an inhibitor of type III receptor tyrosine kinase.

8563. The method of item 8510 wherein the agent is an irreversible inhibitor of enzyme methionine aminopeptidase type 2.

8564. The method of item 8510 wherein the agent is an isozyme selective delta protein kinase C inhibitor.

8565. The method of item 8510 wherein the agent a JAK3 enzyme inhibitor.

8566. The method of item 8510 wherein the agent is a JNK inhibitor.

8567. The method of item 8510 wherein the agent is a kinase inhibitor.

8568. The method of item 8510 wherein the agent is kinesin antagonist.

8569. The method of item 8510 wherein the agent is a kinesin antagonist.

8570. The method of item 8510 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

8571. The method of item 8510 wherein the agent is an MAP kinase inhibitor.

8572. The method of item 8510 wherein the agent is a matrix metalloproteinase inhibitor.

8573. The method of item 8510 wherein the agent is an MCP-CCR2 inhibitor.

8574. The method of item 8510 wherein the agent is an mTOR inhibitor.

8575. The method of item 8510 wherein the agent is an mTOR kinase inhibitor.

8576. The method of item 8510 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

8577. The method of item 8510 wherein the agent is an MIF inhibitor.

8578. The method of item 8510 wherein the agent is an MMP inhibitor.

8579. The method of item 8510 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

8580. The method of item 8510 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

8581. The method of item 8510 wherein the agent is a nitric oxide agonist.

8582. The method of item 8510 wherein the agent is an ornithine decarboxylase inhibitor.

8583. The method of item 8510 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

8584. The method of item 8510 wherein the agent is a palmitoyl-protein thioesterase inhibitor.

8585. The method of item 8510 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

8586. The method of item 8510 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

8587. The method of item 8510 wherein the agent is a phosphatase inhibitor.

8588. The method of item 8510 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

8589. The method of item 8510 wherein the agent is a PKC inhibitor.

8590. The method of item 8510 wherein the agent is a platelet activating factor antagonist.

8591. The method of item 8510 wherein the agent is a platelet-derived growth factor receptor kinase inhibitor.

8592. The method of item 8510 wherein the agent is a prolyl hydroxylase inhibitor.

8593. The method of item 8510 wherein the agent is a polymorphonuclear neutrophil inhibitor.

8594. The method of item 8510 wherein the agent is a protein kinase B inhibitor.

8595. The method of item 8510 wherein the agent is a protein kinase C stimulant.

8596. The method of item 8510 wherein the agent is a purine nucleoside analogue.

8597. The method of item 8510 wherein the agent is a purinoreceptor P2X antagonist.

8598. The method of item 8510 wherein the agent is a Raf kinase inhibitor.

8599. The method of item 8510 wherein the agent is a reversible inhibitor of ErbB1 and ErbB2.

8600. The method of item 8510 wherein the agent is a ribonucleoside triphosphate reductase inhibitor.

8601. The method of item 8510 wherein the agent is an SDF-1 antagonist.

8602. The method of item 8510 wherein the agent is a sheddase inhibitor.

8603. The method of item 8510 wherein the agent is an SRC inhibitor.

8604. The method of item 8510 wherein the agent is a stromelysin inhibitor.

8605. The method of item 8510 wherein the agent is an Syk kinase inhibitor.

8606. The method of item 8510 wherein the agent is a telomerase inhibitor.

8607. The method of item 8510 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

8608. The method of item 8510 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

8609. The method of item 8510 wherein the agent is a Toll receptor inhibitor.

8610. The method of item 8510 wherein the agent is a tubulin antagonist.

8611. The method of item 8510 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her21neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

8612. The method of item 8510 wherein the agent is a VEGF inhibitor.

8613. The method of item 8510 wherein the agent is a vitamin D receptor agonist.

8614. The method of item 8510 wherein the agent is ZD-6474 (an angiogenesis inhibitor).

8615. The method of item 8510 wherein the agent is AP-23573 (an mTOR inhibitor).

8616. The method of item 8510 wherein the agent is synthadotin (a tubulin antagonist).

8617. The method of item 8510 wherein the agent is S-0885 (a collagenase inhibitor).

8618. The method of item 8510 wherein the agent is aplidine (an elongation factor-1 alpha inhibitor).

8619. The method of item 8510 wherein the agent is ixabepilone (an epithilone).

8620. The method of item 8510 wherein the agent is IDN-5390 (an angiogenesis inhibitor).

8621. The method of item 8510 wherein the agent is SB-2723005 (an angiogenesis inhibitor).

8622. The method of item 8510 wherein the agent is ABT-518 (an angiogenesis inhibitor).

8623. The method of item 8510 wherein the agent is combretastatin (an angiogenesis inhibitor).

8624. The method of item 8510 wherein the agent is anecortave acetate (an angiogenesis inhibitor).

8625. The method of item 8510 wherein the agent is SB-715992 (a kinesin antagonist).

8626. The method of item 8510 wherein the agent is temsirolimus (an mTOR inhibitor).

8627. The method of item 8510 wherein the agent is adalimumab (a TNFα antagonist).

8628. The method of item 8510, wherein the composition comprises a polymer.

8629. The method of item 8510, wherein the composition comprises a polymeric carrier.

8630. The method of item 8510 wherein the anti-scarring agent inhibits adhesion between the medical device and a host into which the medical device is implanted.

8631. The method of item 8510 wherein the medical device delivers the anti-scarring agent locally to tissue proximate to the medical device.

8632. The method of item 8510 wherein the medical device has a coating that comprises the anti-scarring agent.

8633. The method of item 8510, wherein the medical device has a coating that comprises the agent and is disposed on a surface of the electrical device.

8634. The method of item 8510, wherein the medical device has a coating that comprises the agent and directly contacts the electrical device.

8635. The method of item 8510, wherein the medical device has a coating that comprises the agent and indirectly contacts the electrical device.

8636. The method of item 8510, wherein the medical device has a coating that comprises the agent and partially covers the electrical device.

8637. The method of item 8510, wherein the medical device has a coating that comprises the agent and completely covers the electrical device.

8638. The method of item 8510, wherein the medical device has a uniform coating.

8639. The method of item 8510, wherein the medical device has a non-uniform coating.

8640. The method of item 8510, wherein the medical device has a discontinuous coating.

8641. The method of item 8510, wherein the medical device has a patterned coating.

8642. The method of item 8510, wherein the medical device has a coating with a thickness of 100 μm or less.

8643. The method of item 8510, wherein the medical device has a coating with a thickness of 10 μm or less.

8644. The method of item 8510, wherein the medical device has a coating, and the coating adheres to the surface of the electrical device upon deployment of the electrical device.

8645. The method of item 8510, wherein the medical device has a coating, and wherein the coating is stable at room temperature for a period of 1 year

8646. The method of item 8510, wherein the medical device has a coating, and wherein the anti-scarring agent is present in the coating in an amount ranging between about 0.0001% to about 1% by weight.

8647. The method of item 8510, wherein the medical device has a coating, and wherein the anti-scarring agent is present in the coating in an amount ranging between about 1% to about 10% by weight

8648. The method of item 8510, wherein the medical device has a coating, and wherein the anti-scarring agent is present in the coating in an amount ranging between about 10% to about 25% by weight.

8649. The method of item 8510, wherein the medical device has a coating, and wherein the anti-scarring agent is present in the coating in an amount ranging between about 25% to about 70% by weight.

8650. The method of item 8510, wherein the medical device has a coating, and wherein the coating further comprises a polymer.

8651. The method of item 8510, wherein the medical device has a first coating having a first composition and a second coating having a second composition.

8652. The method of item 8510, wherein the medical device has a first coating having a first composition and a second coating having a second composition, wherein the first composition and the second composition are different.

8653. The method of item 8510, wherein the composition comprises a polymer.

8654. The method of item 8510, wherein the composition comprises a polymeric carrier.

8655. The method of item 8510, wherein the composition comprises a polymeric carrier, and wherein the polymeric carrier comprises a copolymer.

8656. The method of item 8510, wherein the composition comprises a polymeric carrier, and wherein the polymeric carrier comprises a block copolymer.

8657. The method of item 8510, wherein the composition comprises a polymeric carrier, and wherein the polymeric carrier comprises a random copolymer.

8658. The method of item 8510, wherein the composition comprises a polymeric carrier, and wherein the polymeric carrier comprises a biodegradable polymer.

8659. The method of item 8510, wherein the composition comprises a polymeric carrier, and wherein the polymeric carrier comprises a non-biodegradable polymer.

8660. The method of item 8510, wherein the composition comprises a polymeric carrier, and wherein the polymeric carrier comprises a hydrophilic polymer.

8661. The method of item 8510, wherein the composition comprises a polymeric carrier, and wherein the polymeric carrier comprises a hydrophobic polymer.

8662. The method of item 8510, wherein the composition comprises a polymeric carrier, and wherein the polymeric carrier comprises a polymer having hydrophilic domains.

8663. The method of item 8510, wherein the composition comprises a polymeric carrier, and wherein the polymeric carrier comprises a polymer having hydrophobic domains.

8664. The method of item 8510, wherein the composition comprises a polymeric carrier, and wherein the polymeric carrier comprises a non-conductive polymer.

8665. The method of item 8510, wherein the composition comprises a polymeric carrier, and wherein the polymeric carrier comprises an elastomer.

8666. The method of item 8510, wherein the composition comprises a polymeric carrier, and wherein the polymeric carrier comprises a hydrogel.

8667. The method of item 8510, wherein the composition comprises a polymeric carrier, and wherein the polymeric carrier comprises a silicone polymer.

8668. The method of item 8510, wherein the composition comprises a polymeric carrier, and wherein the polymeric carrier comprises a hydrocarbon polymer.

8669. The method of item 8510, wherein the composition comprises a polymeric carrier, and wherein the polymeric carrier comprises a styrene-derived polymer.

8670. The method of item 8510, wherein the composition comprises a polymeric carrier, and wherein the polymeric carrier comprises a butadiene polymer.

8671. The method of item 8510, wherein the composition comprises a polymeric carrier, and wherein the polymeric carrier comprises a macromer.

8672. The method of item 8510, wherein the composition comprises a polymeric carrier, and wherein the polymeric carrier comprises a poly(ethylene glycol) polymer.

8673. The method of item 8510 wherein the composition comprises a polymeric carrier, and wherein the polymeric carrier comprises an amorphous polymer.

8674. The method of item 8510, wherein the medical device comprises a lubricious coating.

8675. The method of item 8510 wherein the anti-scarring agent is located within pores or holes of the medical device.

8676. The method of item 8510 wherein the anti-scarring agent is located within a channel, lumen, or divet of the medical device.

8677. The method of item 8510, wherein the medical device further comprises a second pharmaceutically active agent.

8678. The method of item 8510 wherein the medical device further comprises an anti-inflammatory agent.

8679. The method of item 8510 wherein the medical device further comprises an agent that inhibits infection.

8680. The method of item 8510 wherein the medical device further comprises an agent that inhibits infection, and wherein the agent is an anthracycline.

8681. The method of item 8510 wherein the medical device further comprises an agent that inhibits infection, and wherein the agent is doxorubicin.

8682. The method of item 8510 wherein the medical device further comprises an agent that inhibits infection, and wherein the agent is mitoxantrone.

8683. The method of item 8510 wherein the medical device further comprises an agent that inhibits infection, and wherein the agent is a fluoropyrimidine.

8684. The method of item 8510 wherein the medical device further comprises an agent that inhibits infection, and wherein the agent is 5-fluorouracil (5-FU).

8685. The method of item 8510 wherein the medical device further comprises an agent that inhibits infection, and wherein the agent is a folic acid antagonist.

8686. The method of item 8510 wherein the medical device further comprises an agent that inhibits infection, and wherein the agent is methotrexate.

8687. The method of item 8510 wherein the medical device further comprises an agent that inhibits infection, and wherein the agent is a podophylotoxin.

8688. The method of item 8510 wherein the medical device further comprises an agent that inhibits infection, and wherein the agent is etoposide.

8689. The method of item 8510 wherein the medical device further comprises an agent that inhibits infection, and wherein the agent is a camptothecin.

8690. The method of item 8510 wherein the medical device further comprises an agent that inhibits infection, and wherein the agent is a hydroxyurea.

8691. The method of item 8510 wherein the medical device further comprises an agent that inhibits infection, and wherein the agent is a platinum complex.

8692. The method of item 8510 wherein the medical device further comprises an agent that inhibits infection, and wherein the agent is cisplatin.

8693. The method of item 8510 wherein the medical device further comprises an anti-thrombotic agent.

8694. The method of item 8510 wherein the medical device further comprises a visualization agent.

8695. The method of item 8510 wherein the medical device further comprises a visualization agent, wherein the visualization agent is a radiopaque material, and wherein the radiopaque material further comprises a metal, a halogenated compound, or a barium containing compound.

8696. The method of item 8510 wherein the medical device further comprises a visualization agent, wherein the visualization agent is a radiopaque material, and wherein the radiopaque material further comprises barium, tantalum, or technetium.

8697. The method of item 8510 wherein the medical device further comprises a visualization agent, and wherein the visualization agent is a MRI responsive material.

8698. The method of item 8510 wherein the medical device further comprises a visualization agent, and wherein the visualization agent further comprises a gadolinium chelate.

8699. The method of item 8510 wherein the medical device further comprises a visualization agent, and wherein the visualization agent further comprises iron, magnesium, manganese, copper, or chromium.

8700. The method of item 8510 wherein the medical device further comprises a visualization agent, and wherein the visualization agent further comprises an iron oxide compound.

8701. The method of item 8510 wherein the medical device further comprises a visualization agent, and wherein the visualization agent further comprises a dye, pigment, or colorant

8702. The method of item 8510 wherein the medical device further comprises an echogenic material.

8703. The method of item 8510 wherein the medical device further comprises an echogenic material, and wherein the echogenic material is in the form of a coating.

8704. The method of item 8510 wherein the medical device is sterile.

8705. The method of item 8510 wherein the anti-scarring agent is released into tissue in the vicinity of the medical device after deployment of the medical device.

8706. The method of item 8510 wherein the anti-scarring agent is released into tissue in the vicinity of the medical device after deployment of the medical device, and wherein the tissue is connective tissue.

8707. The method of item 8510 wherein the anti-scarring agent is released into tissue in the vicinity of the medical device after deployment of the medical device, and wherein the tissue is muscle tissue.

8708. The method of item 8510 wherein the anti-scarring agent is released into tissue in the vicinity of the medical device after deployment of the medical device, and wherein the tissue is nerve tissue.

8709. The method of item 8510 wherein the anti-scarring agent is released into tissue in the vicinity of the medical device after deployment of the medical device, and wherein the tissue is epithelium tissue.

8710. The method of item 8510 wherein the anti-scarring agent is released in effective concentrations from the medical device over a period ranging from the time of deployment of the medical device to about 1 year.

8711. The method of item 8510 wherein the anti-scarring agent is released in effective concentrations from the medical device over a period ranging from about 1 month to 6 months.

8712. The method of item 8510 wherein the anti-scarring agent is released in effective concentrations from the medical device over a period ranging from about 1-90 days.

8713. The method of item 8510 wherein the anti-scarring agent is released in effective concentrations from the medical device at a constant rate.

8714. The method of item 8510 wherein the anti-scarring agent is released in effective concentrations from the medical device at an increasing rate.

8715. The method of item 8510 wherein the anti-scarring agent is released in effective concentrations from the medical device at a decreasing rate.

8716. The method of item 8510 wherein the anti-scarring agent is released in effective concentrations from the composition comprising the anti-scarring agent by diffusion over a period ranging from the time of deployment of the medical device to about 90 days

8717. The method of item 8510 wherein the anti-scarring agent is released in effective concentrations from the composition comprising the anti-scarring agent by erosion of the composition over a period ranging from the time of deployment of the medical device to about 90 days.

8718. The method of item 8510 wherein the medical device comprises about 0.01 μg to about 10 μg of the anti-scarring agent.

8719. The method of item 8510 wherein the medical device comprises about 10 μg to about 10 mg of the anti-scarring agent.

8720. The method of item 8510 wherein the medical device comprises about 10 mg to about 250 mg of the anti-scarring agent.

8721. The method of item 8510 wherein the medical device comprises about 250 mg to about 1000 mg of the anti-scarring agent.

8722. The method of item 8510 wherein the medical device comprises about 1000 mg to about 2500 mg of the anti-scarring agent.

8723. The method of item 8510 wherein a surface of the medical device comprises less than 0.01 μg of the anti-scarring agent per mm² of medical device surface to which the anti-scarring agent is applied.

8724. The method of item 8510 wherein a surface of the medical device comprises about 0.01 μg to about 1 μg of the anti-scarring agent per mm² of medical device surface to which the anti-scarring agent is applied.

8725. The method of item 8510 wherein a surface of the medical device comprises about 1 μg to about 10 μg of the anti-scarring agent per mm² of medical device surface to which the anti-scarring agent is applied.

8726. The method of item 8510 wherein a surface of the medical device comprises about 10 μg to about 250 μg of the anti-scarring agent per mm² of medical device surface to which the anti-scarring agent is applied.

8727. The method of item 8510 wherein a surface of the medical device comprises about 250 μg to about 1000 μg of the anti-scarring agent of anti-scarring agent per mm² of medical device surface to which the anti-scarring agent is applied.

8728. The method of item 8510 wherein a surface of the medical device comprises about 1000 μg to about 2500 μg of the anti-scarring agent per mm² of medical device surface to which the anti-scarring agent is applied.

8729. The method of item 8510 wherein the combining is performed by direct affixing the agent or the composition to the electrical device.

8730. The method of item 8510 wherein the combining is performed by spraying the agent or the component onto the electrical device.

8731. The method of item 8510 wherein the combining is performed by electrospraying the agent or the composition onto the electrical device.

8732. The method of item 8510 wherein the combining is performed by dipping the electrical device into a solution comprising the agent or the composition.

8733. The method of item 8510 wherein the combining is performed by covalently attaching the agent or the composition to the electrical device.

8734. The method of item 8510 wherein the combining is performed by non-covalently attaching the agent or the composition to the electrical device.

8735. The method of item 8510 wherein the combining is performed by coating the electrical device with a substance that contains the agent or the composition.

8736. The method of item 8510 wherein the combining is performed by coating the electrical device with a substance that absorbs the agent.

8737. The method of item 8510 wherein the combining is performed by interweaving the electrical device with a thread composed of, or coated with, the agent or the composition.

8738. The method of item 8510 wherein the combining is performed by completely covering the electrical device with a sleeve that contains the agent or the composition.

8739. The method of item 8510 wherein the combining is performed by covering a portion of the electrical device with a sleeve that contains the agent or the composition.

8740. The method of item 8510 wherein the combining is performed by completely covering the electrical device with a cover that contains the agent or the composition.

8741. The method of item 8510 wherein the combining is performed by covering a portion of the electrical device with a cover that contains the agent or the composition.

8742. The method of item 8510 wherein the combining is performed by completely covering the electrical device with an electrospun fabric that contains the agent or the composition.

8743. The method of item 8510 wherein the combining is performed by covering a portion of the electrical device with an electrospun fabric that contains the agent or the composition.

8744. The method of item 8510 wherein the combining is performed by completely covering the electrical device with a mesh that contains the agent or the composition.

8745. The method of item 8510 wherein the combining is performed by covering a portion of the electrical device with a mesh that contains the agent or the composition.

8746. The method of item 8510 wherein the combining is performed by constructing a portion of the electrical device with the agent or the composition.

8747. The method of item 8510 wherein the combining is performed by impregnating the electrical device with the agent or the composition.

8748. The method of item 8510 wherein the combining is performed by constructing a portion of the electrical device from a degradable polymer that releases the agent.

8749. The method of item 8510 wherein the combining is performed by dipping the electrical device into a solution that comprise the agent and an inert solvent for the electrical device.

8750. The method of item 8510 wherein the combining is performed by dipping the electrical device into a solution that comprises the agent and a solvent that will swell the electrical device.

8751. The method of item 8510 wherein the combining is performed by dipping the electrical device into a solution that comprises the agent and a solvent that will dissolve the electrical device.

8752. The method of item 8510 wherein the combining is performed by dipping the electrical device into a solution that comprises the agent, a polymer and an inert solvent for the electrical device.

8753. The method of item 8510 wherein the combining is performed by dipping the electrical device into a solution that comprises the agent, a polymer and a solvent that will swell the electrical device.

8754. The method of item 8510 wherein the combining is performed by dipping the electrical device into a solution that comprises the agent, a polymer and a solvent that will dissolve the electrical device.

8755. The method of item 8510 wherein the combining is performed by spraying the electrical device into a solution that comprises the agent and an inert solvent for the electrical device.

8756. The method of item 8510 wherein the combining is performed by spraying the electrical device into a solution that comprises the agent and a solvent that will swell the electrical device.

8757. The method of item 8510 wherein the combining is performed by spraying the electrical device into a solution that comprises the agent and a solvent that will dissolve the electrical device.

8758. The method of item 8510 wherein the combining is performed by spraying the electrical device into a solution that comprises the agent, a polymer and an inert solvent for the electrical device.

8759. The method of item 8510 wherein the combining is performed by spraying the electrical device into a solution that comprises the agent, a polymer and a solvent that will swell the electrical device.

8760. The method of item 8510 wherein the combining is performed by spraying the electrical device into a solution that comprises the agent, a polymer and a solvent that will dissolve the electrical device.

8761. The method for making a medical device of any one of items 8510-8760 wherein the vagal nerve stimulator is adapted for treating or preventing depression.

8762. The method for making a medical device of any one of items 8510-8760 wherein the vagal nerve stimulator is adapted for treating or preventing anxiety.

8763. The method for making a medical device of any one of items 8510-8760 wherein the vagal nerve stimulator is adapted for treating or preventing panic disorders.

8764. The method for making a medical device of any one of items 8510-8760 wherein the vagal nerve stimulator is adapted for treating or preventing obsessive-compulsive disorders.

8765. The method for making a medical device of any one of items 8510-8760 wherein the vagal nerve stimulator is adapted for treating or preventing post-traumatic disorders.

8766. The method for making a medical device of any one of items 8510-8760 wherein the vagal nerve stimulator is adapted for treating or preventing obesity.

8767. The method for making a medical device of any one of items 8510-8760 wherein the vagal nerve stimulator is adapted for treating or preventing migraine.

8768. The method for making a medical device of any one of items 8510-8760 wherein the vagal nerve stimulator is adapted for treating or preventing sleep disorders.

8769. The method for making a medical device of any one of items 8510-8760 wherein the vagal nerve stimulator is adapted for treating or preventing dementia.

8770. The method for making a medical device of any one of items 8510-8760 wherein the vagal nerve stimulator is adapted for treating or preventing Alzheimer's disease.

8771. The method for making a medical device of any one of items 8510-8760 wherein the vagal nerve stimulator is adapted for treating or preventing chronic or degenerative neurological disorders.

8772. A method for making a medical device comprising: combining a sacral nerve stimulator for treating a bladder control problem (i.e., an electrical device) and an anti-scarring agent or a composition comprising an anti-scarring agent, wherein the agent inhibits scarring between the device and a host into which the device is implanted.

8773. The method of item 8772 wherein the agent is an adensosine A2A receptor antagonist.

8774. The method of item 8772 wherein the agent is an AKT inhibitor.

8775. The method of item 8772 wherein the agent is an alpha 2 integrin antagonist, wherein the alpha 2 integrin antagonist is Pharmaprojects No. 5754 (Merck KgaA).

8776. The method of item 8772 wherein the agent is an alpha 4 integrin antagonist

8777. The method of item 8772 wherein the agent is an alpha 7 nicotinic receptor agonist.

8778. The method of item 8772 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

8779. The method of item 8772 wherein the agent is an apoptosis antagonist.

8780. The method of item 8772 wherein the agent is an apoptosis activator.

8781. The method of item 8772 wherein the agent is a beta 1 integrin antagonist.

8782. The method of item 8772 wherein the agent is a beta tubulin inhibitor.

8783. The method of item 8772 wherein the agent is a blocker of enzyme production in Hepatitis C.

8784. The method of item 8772 wherein the agent is a Bruton's tyrosine kinase inhibitor.

8785. The method of item 8772 wherein the agent is a calcineurin inhibitor.

8786. The method of item 8772 wherein the agent is a caspase 3 inhibitor.

8787. The method of item 8772 wherein the agent is a CC chemokine receptor antagonist.

8788. The method of item 8772 wherein the agent is a cell cycle inhibitor selected from the group consisting of SNS-595 (Sunesis), synthadotin, KRX-0403, and an analogue or derivative thereof.

8789. The method of item 8772 wherein the agent is a cathepsin B inhibitor.

8790. The method of item 8772 wherein the agent is a cathepsin K inhibitor, wherein the cathepsin K inhibitor is 462795 (GlaxoSmithKline), INPL-022-D6 (Amura Therapeutics), or an analogue or derivative thereof.

8791. The method of item 8772 wherein the agent is a cathepsin L inhibitor.

8792. The method of item 8772 wherein the agent is a CD40 antagonist.

8793. The method of item 8772 wherein the agent is a chemokine receptor agonist.

8794. The method of item 8772 wherein the agent is a chymase inhibitor.

8795. The method of item 8772 wherein the agent is a collagenase antagonist.

8796. The method of item 8772 wherein the agent is a CXCR antagonist.

8797. The method of item 8772 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

8798. The method of item 8772 wherein the agent is a cyclooxygenase 1 inhibitor.

8799. The method of item 8772 wherein the agent is a DHFR inhibitor.

8800. The method of item 8772 wherein the agent is a dual integrin inhibitor.

8801. The method of item 8772 wherein the agent is an elastase inhibitor.

8802. The method of item 8772 wherein the agent is an elongation factor-1 alpha inhibitor.

8803. The method of item 8772 wherein the agent is an endothelial growth factor antagonist.

8804. The method of item 8772 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

8805. The method of item 8772 wherein the agent is an endotoxin antagonist.

8806. The method of item 8772 wherein the agent is an epothilone and tubulin binder.

8807. The method of item 8772 wherein the agent is an estrogen receptor antagonist.

8808. The method of item 8772 wherein the agent is an FGF inhibitor.

8809. The method of item 8772 wherein the agent is a farnexyl transferase inhibitor.

8810. The method of item 8772 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

8811. The method of item 8772 wherein the agent is an FLT-3 kinase inhibitor.

8812. The method of item 8772 wherein the agent is an FGF receptor kinase inhibitor.

8813. The method of item 8772 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

8814. The method of item 8772 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

8815. The method of item 8772 wherein the agent is a histone deacetylase inhibitor.

8816. The method of item 8772 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

8817. The method of item 8772 wherein the agent is an ICAM inhibitor.

8818. The method of item 8772 wherein the agent is an IL, ICE and IRAK antagonist, wherein the antagonist is a CJ-14877, CP-424174 (Pfizer), NF-61 (Negma-Lerads), and an analogue or derivative thereof.

8819. The method of item 8772 wherein the agent is an IL-2 inhibitor.

8820. The method of item 8772 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

8821. The method of item 8772 wherein the agent is an IMPDH (inosine monophosphate).

8822. The method of item 8772 wherein the agent is an integrin antagonist.

8823. The method of item 8772 wherein the agent is an interleukin antagonist.

8824. The method of item 8772 wherein the agent is an inhibitor of type III receptor tyrosine kinase.

8825. The method of item 8772 wherein the agent is an irreversible inhibitor of enzyme methionine aminopeptidase type 2.

8826. The method of item 8772 wherein the agent is an isozyme selective delta protein kinase C inhibitor.

8827. The method of item 8772 wherein the agent a JAK3 enzyme inhibitor.

8828. The method of item 8772 wherein the agent is a JNK inhibitor.

8829. The method of item 8772 wherein the agent is a kinase inhibitor.

8830. The method of item 8772 wherein the agent is kinesin antagonist.

8831. The method of item 8772 wherein the agent is a kinesin antagonist.

8832. The method of item 8772 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

8833. The method of item 8772 wherein the agent is an MAP kinase inhibitor.

8834. The method of item 8772 wherein the agent is a matrix metalloproteinase inhibitor.

8835. The method of item 8772 wherein the agent is an MCP-CCR2 inhibitor.

8836. The method of item 8772 wherein the agent is an mTOR inhibitor.

8837. The method of item 8772 wherein the agent is an mTOR kinase inhibitor.

8838. The method of item 8772 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

8839. The method of item 8772 wherein the agent is an MIF inhibitor.

8840. The method of item 8772 wherein the agent is an MMP inhibitor.

8841. The method of item 8772 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

8842. The method of item 8772 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

8843. The method of item 8772 wherein the agent is a nitric oxide agonist.

8844. The method of item 8772 wherein the agent is an ornithine decarboxylase inhibitor.

8845. The method of item 8772 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

8846. The method of item 8772 wherein the agent is a palmitoyl-protein thioesterase inhibitor.

8847. The method of item 8772 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

8848. The method of item 8772 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

8849. The method of item 8772 wherein the agent is a phosphatase inhibitor.

8850. The method of item 8772 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

8851. The method of item 8772 wherein the agent is a PKC inhibitor.

8852. The method of item 8772 wherein the agent is a platelet activating factor antagonist.

8853. The method of item 8772 wherein the agent is a platelet-derived growth factor receptor kinase inhibitor.

8854. The method of item 8772 wherein the agent is a prolyl hydroxylase inhibitor.

8855. The method of item 8772 wherein the agent is a polymorphonuclear neutrophil inhibitor.

8856. The method of item 8772 wherein the agent is a protein kinase B inhibitor.

8857. The method of item 8772 wherein the agent is a protein kinase C stimulant.

8858. The method of item 8772 wherein the agent is a purine nucleoside analogue.

8859. The method of item 8772 wherein the agent is a purinoreceptor P2X antagonist.

8860. The method of item 8772 wherein the agent is a Raf kinase inhibitor.

8861. The method of item 8772 wherein the agent is a reversible inhibitor of ErbB1 and ErbB2.

8862. The method of item 8772 wherein the agent is a ribonucleoside triphosphate reductase inhibitor.

8863. The method of item 8772 wherein the agent is an SDF-1 antagonist.

8864. The method of item 8772 wherein the agent is a sheddase inhibitor.

8865. The method of item 8772 wherein the agent is an SRC inhibitor.

8866. The method of item 8772 wherein the agent is a stromelysin inhibitor.

8867. The method of item 8772 wherein the agent is an Syk kinase inhibitor.

8868. The method of item 8772 wherein the agent is a telomerase inhibitor.

8869. The method of item 8772 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

8870. The method of item 8772 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-1 38 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

8871. The method of item 8772 wherein the agent is a Toll receptor inhibitor.

8872. The method of item 8772 wherein the agent is a tubulin antagonist.

8873. The method of item 8772 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-112 0 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

8874. The method of item 8772 wherein the agent is a VEGF inhibitor.

8875. The method of item 8772 wherein the agent is a vitamin D receptor agonist.

8876. The method of item 8772 wherein the agent is ZD-6474 (an angiogenesis inhibitor).

8877. The method of item 8772 wherein the agent is AP-23573 (an mTOR inhibitor).

8878. The method of item 8772 wherein the agent is synthadotin (a tubulin antagonist).

8879. The method of item 8772 wherein the agent is S-0885 (a collagenase inhibitor).

8880. The method of item 8772 wherein the agent is aplidine (an elongation factor-1 alpha inhibitor).

8881. The method of item 8772 wherein the agent is ixabepilone (an epithilone).

8882. The method of item 8772 wherein the agent is IDN-5390 (an angiogenesis inhibitor).

8883. The method of item 8772 wherein the agent is SB-2723005 (an angiogenesis inhibitor).

8884. The method of item 8772 wherein the agent is ABT-518 (an angiogenesis inhibitor).

8885. The method of item 8772 wherein the agent is combretastatin (an angiogenesis inhibitor).

8886. The method of item 8772 wherein the agent is anecortave acetate (an angiogenesis inhibitor).

8887. The method of item 8772 wherein the agent is SB-715992 (a kinesin antagonist).

8888. The method of item 8772 wherein the agent is temsirolimus (an mTOR inhibitor).

8889. The method of item 8772 wherein the agent is adalimumab (a TNFα antagonist).

8890. The method of item 8772, wherein the composition comprises a polymer.

8891. The method of item 8772, wherein the composition comprises a polymeric carrier.

8892. The method of item 8772 wherein the anti-scarring agent inhibits adhesion between the medical device and a host into which the medical device is implanted.

8893. The method of item 8772 wherein the medical device delivers the anti-scarring agent locally to tissue proximate to the medical device.

8894. The method of item 8772 wherein the medical device has a coating that comprises the anti-scarring agent.

8895. The method of item 8772, wherein the medical device has a coating that comprises the agent and is disposed on a surface of the electrical device.

8896. The method of item 8772, wherein the medical device has a coating that comprises the agent and directly contacts the electrical device.

8897. The method of item 8772, wherein the medical device has a coating that comprises the agent and indirectly contacts the electrical device.

8898. The method of item 8772, wherein the medical device has a coating that comprises the agent and partially covers the electrical device.

8899. The method of item 8772, wherein the medical device has a coating that comprises the agent and completely covers the electrical device.

8900. The method of item 8772, wherein the medical device has a uniform coating.

8901. The method of item 8772, wherein the medical device has a non-uniform coating.

8902. The method of item 8772, wherein the medical device has a discontinuous coating.

8903. The method of item 8772, wherein the medical device has a patterned coating.

8904. The method of item 8772, wherein the medical device has a coating with a thickness of 100 μm or less.

8905. The method of item 8772, wherein the medical device has a coating with a thickness of 10 μm or less.

8906. The method of item 8772, wherein the medical device has a coating, and the coating adheres to the surface of the electrical device upon deployment of the electrical device.

8907. The method of item 8772, wherein the medical device has a coating, and wherein the coating is stable at room temperature for a period of 1 year.

8908. The method of item 8772, wherein the medical device has a coating, and wherein the anti-scarring agent is present in the coating in an amount ranging between about 0.0001% to about 1% by weight.

8909. The method of item 8772, wherein the medical device has a coating, and wherein the anti-scarring agent is present in the coating in an amount ranging between about 1% to about 10% by weight.

8910. The method of item 8772, wherein the medical device has a coating, and wherein the anti-scarring agent is present in the coating in an amount ranging between about 10% to about 25% by weight.

8911. The method of item 8772, wherein the medical device has a coating, and wherein the anti-scarring agent is present in the coating in an amount ranging between about 25% to about 70% by weight.

8912. The method of item 8772, wherein the medical device has a coating, and wherein the coating further comprises a polymer.

8913. The method of item 8772, wherein the medical device has a first coating having a first composition and a second coating having a second composition.

8914. The method of item 8772, wherein the medical device has a first coating having a first composition and a second coating having a second composition, wherein the first composition and the second composition are different.

8915. The method of item 8772, wherein the composition comprises a polymer.

8916. The method of item 8772, wherein the composition comprises a polymeric carrier.

8917. The method of item 8772, wherein the composition comprises a polymeric carrier, and wherein the polymeric carrier comprises a copolymer.

8918. The method of item 8772, wherein the composition comprises a polymeric carrier, and wherein the polymeric carrier comprises a block copolymer.

8919. The method of item 8772, wherein the composition comprises a polymeric carrier, and wherein the polymeric carrier comprises a random copolymer.

8920. The method of item 8772, wherein the composition comprises a polymeric carrier, and wherein the polymeric carrier comprises a biodegradable polymer.

8921. The method of item 8772, wherein the composition comprises a polymeric carrier, and wherein the polymeric carrier comprises a non-biodegradable polymer.

8922. The method of item 8772, wherein the composition comprises a polymeric carrier, and wherein the polymeric carrier comprises a hydrophilic polymer.

8923. The method of item 8772, wherein the composition comprises a polymeric carrier, and wherein the polymeric carrier comprises a hydrophobic polymer.

8924. The method of item 8772, wherein the composition comprises a polymeric carrier, and wherein the polymeric carrier comprises a polymer having hydrophilic domains.

8925. The method of item 8772, wherein the composition comprises a polymeric carrier, and wherein the polymeric carrier comprises a polymer having hydrophobic domains.

8926. The method of item 8772, wherein the composition comprises a polymeric carrier, and wherein the polymeric carrier comprises a non-conductive polymer.

8927. The method of item 8772, wherein the composition comprises a polymeric carrier, and wherein the polymeric carrier comprises an elastomer.

8928. The method of item 8772, wherein the composition comprises a polymeric carrier, and wherein the polymeric carrier comprises a hydrogel

8929. The method of item 8772, wherein the composition comprises a polymeric carrier, and wherein the polymeric carrier comprises a silicone polymer.

8930. The method of item 8772, wherein the composition comprises a polymeric carrier, and wherein the polymeric carrier comprises a hydrocarbon polymer.

8931. The method of item 8772, wherein the composition comprises a polymeric carrier, and wherein the polymeric carrier comprises a styrene-derived polymer.

8932. The method of item 8772, wherein the composition comprises a polymeric carrier, and wherein the polymeric carrier comprises a butadiene polymer.

8933. The method of item 8772, wherein the composition comprises a polymeric carrier, and wherein the polymeric carrier comprises a macromer.

8934. The method of item 8772, wherein the composition comprises a polymeric carrier, and wherein the polymeric carrier comprises a poly(ethylene glycol) polymer.

8935. The method of item 8772 wherein the composition comprises a polymeric carrier, and wherein the polymeric carrier comprises an amorphous polymer.

8936. The method of item 8772, wherein the medical device comprises a lubricious coating.

8937. The method of item 8772 wherein the anti-scarring agent is located within pores or holes of the medical device.

8938. The method of item 8772 wherein the anti-scarring agent is located within a channel, lumen, or divet of the medical device.

8939. The method of item 8772, wherein the medical device further comprises a second pharmaceutically active agent.

8940. The method of item 8772 wherein the medical device further comprises an anti-inflammatory agent.

8941. The method of item 8772 wherein the medical device further comprises an agent that inhibits infection.

8942. The method of item 8772 wherein the medical device further comprises an agent that inhibits infection, and wherein the agent is an anthracycline.

8943. The method of item 8772 wherein the medical device further comprises an agent that inhibits infection, and wherein the agent is doxorubicin.

8944. The method of item 8772 wherein the medical device further comprises an agent that inhibits infection, and wherein the agent is mitoxantrone.

8945. The method of item 8772 wherein the medical device further comprises an agent that inhibits infection, and wherein the agent is a fluoropyrimidine.

8946. The method of item 8772 wherein the medical device further comprises an agent that inhibits infection, and wherein the agent is 5-fluorouracil (5-FU).

8947. The method of item 8772 wherein the medical device further comprises an agent that inhibits infection, and wherein the agent is a folic acid antagonist.

8948. The method of item 8772 wherein the medical device further comprises an agent that inhibits infection, and wherein the agent is methotrexate.

8949. The method of item 8772 wherein the medical device further comprises an agent that inhibits infection, and wherein the agent is a podophylotoxin.

8950. The method of item 8772 wherein the medical device further comprises an agent that inhibits infection, and wherein the agent is etoposide.

8951. The method of item 8772 wherein the medical device further comprises an agent that inhibits infection, and wherein the agent is a camptothecin.

8952. The method of item 8772 wherein the medical device further comprises an agent that inhibits infection, and wherein the agent is a hydroxyurea.

8953. The method of item 8772 wherein the medical device further comprises an agent that inhibits infection, and wherein the agent is a platinum complex.

8954. The method of item 8772 wherein the medical device further comprises an agent that inhibits infection, and wherein the agent is cisplatin.

8955. The method of item 8772 wherein the medical device further comprises an anti-thrombotic agent.

8956. The method of item 8772 wherein the medical device further comprises a visualization agent.

8957. The method of item 8772 wherein the medical device further comprises a visualization agent, wherein the visualization agent is a radiopaque material, and wherein the radiopaque material further comprises a metal, a halogenated compound, or a barium containing compound

8958. The method of item 8772 wherein the medical device further comprises a visualization agent, wherein the visualization agent is a radiopaque material, and wherein the radiopaque material further comprises barium, tantalum, or technetium.

8959. The method of item 8772 wherein the medical device further comprises a visualization agent, and wherein the visualization agent is a MRI responsive material.

8960. The method of item 8772 wherein the medical device further comprises a visualization agent, and wherein the visualization agent further comprises a gadolinium chelate.

8961. The method of item 8772 wherein the medical device further comprises a visualization agent, and wherein the visualization agent further comprises iron, magnesium, manganese, copper, or chromium.

8962. The method of item 8772 wherein the medical device further comprises a visualization agent, and wherein the visualization agent further comprises an iron oxide compound.

8963. The method of item 8772 wherein the medical device further comprises a visualization agent, and wherein the visualization agent further comprises a dye, pigment, or colorant.

8964. The method of item 8772 wherein the medical device further comprises an echogenic material.

8965. The method of item 8772 wherein the medical device further comprises an echogenic material, and wherein the echogenic material is in the form of a coating.

8966. The method of item 8772 wherein the medical device is sterile.

8967. The method of item 8772 wherein the anti-scarring agent is released into tissue in the vicinity of the medical device after deployment of the medical device.

8968. The method of item 8772 wherein the anti-scarring agent is released into tissue in the vicinity of the medical device after deployment of the medical device, and wherein the tissue is connective tissue.

8969. The method of item 8772 wherein the anti-scarring agent is released into tissue in the vicinity of the medical device after deployment of the medical device, and wherein the tissue is muscle tissue.

8970. The method of item 8772 wherein the anti-scarring agent is released into tissue in the vicinity of the medical device after deployment of the medical device, and wherein the tissue is nerve tissue.

8971. The method of item 8772 wherein the anti-scarring agent is released into tissue in the vicinity of the medical device after deployment of the medical device, and wherein the tissue is epithelium tissue.

8972. The method of item 8772 wherein the anti-scarring agent is released in effective concentrations from the medical device over a period ranging from the time of deployment of the medical device to about 1 year.

8973. The method of item 8772 wherein the anti-scarring agent is released in effective concentrations from the medical device over a period ranging from about 1 month to 6 months.

8974. The method of item 8772 wherein the anti-scarring agent is released in effective concentrations from the medical device over a period ranging from about 1-90 days.

8975. The method of item 8772 wherein the anti-scarring agent is released in effective concentrations from the medical device at a constant rate.

8976. The method of item 8772 wherein the anti-scarring agent is released in effective concentrations from the medical device at an increasing rate.

8977. The method of item 8772 wherein the anti-scarring agent is released in effective concentrations from the medical device at a decreasing rate.

8978. The method of item 8772 wherein the anti-scarring agent is released in effective concentrations from the composition comprising the anti-scarring agent by diffusion over a period ranging from the time of deployment of the medical device to about 90 days.

8979. The method of item 8772 wherein the anti-scarring agent is released in effective concentrations from the composition comprising the anti-scarring agent by erosion of the composition over a period ranging from the time of deployment of the medical device to about 90 days.

8980. The method of item 8772 wherein the medical device comprises about 0.01 μg to about 10 μg of the anti-scarring agent.

8981. The method of item 8772 wherein the medical device comprises about 10 μg to about 10 mg of the anti-scarring agent.

8982. The method of item 8772 wherein the medical device comprises about 10 mg to about 250 mg of the anti-scarring agent.

8983. The method of item 8772 wherein the medical device comprises about 250 mg to about 1000 mg of the anti-scarring agent.

8984. The method of item 8772 wherein the medical device comprises about 1000 mg to about 2500 mg of the anti-scarring agent.

8985. The method of item 8772 wherein a surface of the medical device comprises less than 0.01 μg of the anti-scarring agent per mm² of medical device surface to which the anti-scarring agent is applied.

8986. The method of item 8772 wherein a surface of the medical device comprises about 0.01 μg to about 1 μg of the anti-scarring agent per mm² of medical device surface to which the anti-scarring agent is applied.

8987. The method of item 8772 wherein a surface of the medical device comprises about 1 μg to about 10 μg of the anti-scarring agent per mm² of medical device surface to which the anti-scarring agent is applied.

8988. The method of item 8772 wherein a surface of the medical device comprises about 10 μg to about 250 μg of the anti-scarring agent per mm² of medical device surface to which the anti-scarring agent is applied.

8989. The method of item 8772 wherein a surface of the medical device comprises about 250 μg to about 1000 μg of the anti-scarring agent of anti-scarring agent per mm² of medical device surface to which the anti-scarring agent is applied.

8990. The method of item 8772 wherein a surface of the medical device comprises about 1000 μg to about 2500 μg of the anti-scarring agent per mm² of medical device surface to which the anti-scarring agent is applied.

8991. The method of item 8772 wherein the combining is performed by direct affixing the agent or the composition to the electrical device.

8992. The method of item 8772 wherein the combining is performed by spraying the agent or the component onto the electrical device.

8993. The method of item 8772 wherein the combining is performed by electrospraying the agent or the composition onto the electrical device.

8994. The method of item 8772 wherein the combining is performed by dipping the electrical device into a solution comprising the agent or the composition.

8995. The method of item 8772 wherein the combining is performed by covalently attaching the agent or the composition to the electrical device.

8996. The method of item 8772 wherein the combining is performed by non-covalently attaching the agent or the composition to the electrical device.

8997. The method of item 8772 wherein the combining is performed by coating the electrical device with a substance that contains the agent or the composition.

8998. The method of item 8772 wherein the combining is performed by coating the electrical device with a substance that absorbs the agent.

8999. The method of item 8772 wherein the combining is performed by interweaving the electrical device with a thread composed of, or coated with, the agent or the composition.

9000. The method of item 8772 wherein the combining is performed by completely covering the electrical device with a sleeve that contains the agent or the composition.

9001. The method of item 8772 wherein the combining is performed by covering a portion of the electrical device with a sleeve that contains the agent or the composition.

9002. The method of item 8772 wherein the combining is performed by completely covering the electrical device with a cover that contains the agent or the composition.

9003. The method of item 8772 wherein the combining is performed by covering a portion of the electrical device with a cover that contains the agent or the composition.

9004. The method of item 8772 wherein the combining is performed by completely covering the electrical device with an electrospun fabric that contains the agent or the composition.

9005. The method of item 8772 wherein the combining is performed by covering a portion of the electrical device with an electrospun fabric that contains the agent or the composition.

9006. The method of item 8772 wherein the combining is performed by completely covering the electrical device with a mesh that contains the agent or the composition.

9007. The method of item 8772 wherein the combining is performed by covering a portion of the electrical device with a mesh that contains the agent or the composition.

9008. The method of item 8772 wherein the combining is performed by constructing a portion of the electrical device with the agent or the composition.

9009. The method of item 8772 wherein the combining is performed by impregnating the electrical device with the agent or the composition.

9010. The method of item 8772 wherein the combining is performed by constructing a portion of the electrical device from a degradable polymer that releases the agent.

9011. The method of item 8772 wherein the combining is performed by dipping the electrical device into a solution that comprise the agent and an inert solvent for the electrical device.

9012. The method of item 8772 wherein the combining is performed by dipping the electrical device into a solution that comprises the agent and a solvent that will swell the electrical device.

9013. The method of item 8772 wherein the combining is performed by dipping the electrical device into a solution that comprises the agent and a solvent that will dissolve the electrical device.

9014. The method of item 8772 wherein the combining is performed by dipping the electrical device into a solution that comprises the agent a polymer and an inert solvent for the electrical device.

9015. The method of item 8772 wherein the combining is performed by dipping the electrical device into a solution that comprises the agent, a polymer and a solvent that will swell the electrical device.

9016. The method of item 8772 wherein the combining is performed by dipping the electrical device into a solution that comprises the agent, a polymer and a solvent that will dissolve the electrical device.

9017. The method of item 8772 wherein the combining is performed by spraying the electrical device into a solution that comprises the agent and an inert solvent for the electrical device.

9018. The method of item 8772 wherein the combining is performed by spraying the electrical device into a solution that comprises the agent and a solvent that will swell the electrical device.

9019. The method of item 8772 wherein the combining is performed by spraying the electrical device into a solution that comprises the agent and a solvent that will dissolve the electrical device.

9020. The method of item 8772 wherein the combining is performed by spraying the electrical device into a solution that comprises the agent, a polymer and an inert solvent for the electrical device.

9021. The method of item 8772 wherein the combining is performed by spraying the electrical device into a solution that comprises the agent, a polymer and a solvent that will swell the electrical device.

9022. The method of item 8772 wherein the combining is performed by spraying the electrical device into a solution that comprises the agent, a polymer and a solvent that will dissolve the electrical device.

9023. The method for making a medical device of any one of items 8772-9022 wherein the sacral nerve stimulator is adapted for treating or preventing urge incontinence.

9024. The method for making a medical device of any one of items 8772-9022 wherein the sacral nerve stimulator is adapted for treating or preventing nonobstructive urinary retention.

9025. The method for making a medical device of any one of items 8772-9022 wherein the sacral nerve stimulator is adapted for treating or preventing urgency frequency.

9026. The method for making a medical device of any one of items 8772-9022 wherein the sacral nerve stimulator is an intramuscular electrical stimulator.

9027. The method for making a medical device of any one of items 8772-9022 wherein the sacral nerve stimulator is a leadless, tubular-shaped microstimulator.

9028. A method for making a medical device comprising: combining a gastric nerve stimulator for treating a gastrointestinal disorder (i.e., an electrical device) and an anti-scarring agent or a composition comprising an anti-scarring agent, wherein the agent inhibits scarring between the device and a host into which the device is implanted.

9029. The method of item 9028 wherein the agent is an adensosine A2A receptor antagonist.

9030. The method of item 9028 wherein the agent is an AKT inhibitor.

9031. The method of item 9028 wherein the agent is an alpha 2 integrin antagonist, wherein the alpha 2 integrin antagonist is Pharmaprojects No. 5754 (Merck KgaA).

9032. The method of item 9028 wherein the agent is an alpha 4 integrin antagonist.

9033. The method of item 9028 wherein the agent is an alpha 7 nicotinic receptor agonist.

9034. The method of item 9028 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-27,23005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

9035. The method of item 9028 wherein the agent is an apoptosis antagonist

9036. The method of item 9028 wherein the agent is an apoptosis activator.

9037. The method of item 9028 wherein the agent is a beta 1 integrin antagonist.

9038. The method of item 9028 wherein the agent is a beta tubulin inhibitor.

9039. The method of item 9028 wherein the agent is a blocker of enzyme production in Hepatitis C.

9040. The method of item 9028 wherein the agent is a Bruton's tyrosine kinase inhibitor.

9041. The method of item 9028 wherein the agent is a calcineurin inhibitor.

9042. The method of item 9028 wherein the agent is a caspase 3 inhibitor.

9043. The method of item 9028 wherein the agent is a CC chemokine receptor antagonist.

9044. The method of item 9028 wherein the agent is a cell cycle inhibitor selected from the group consisting of SNS-595 (Sunesis), synthadotin, KRX-0403, and an analogue or derivative thereof.

9045. The method of item 9028 wherein the agent is a cathepsin B inhibitor.

9046. The method of item 9028 wherein the agent is a cathepsin K inhibitor, wherein the cathepsin K inhibitor is 462795 (GlaxoSmithKline), INPL-022-D6 (Amura Therapeutics), or an analogue or derivative thereof.

9047. The method of item 9028 wherein the agent is a cathepsin L inhibitor.

9048. The method of item 9028 wherein the agent is a CD40 antagonist.

9049. The method of item 9028 wherein the agent is a chemokine receptor agonist.

9050. The method of item 9028 wherein the agent is a chymase inhibitor.

9051. The method of item 9028 wherein the agent is a collagenase antagonist.

9052. The method of item 9028 wherein the agent is a CXCR antagonist.

9053. The method of item 9028 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

9054. The method of item 9028 wherein the agent is a cyclooxygenase 1 inhibitor.

9055. The method of item 9028 wherein the agent is a DHFR inhibitor.

9056. The method of item 9028 wherein the agent is a dual integrin inhibitor.

9057. The method of item 9028 wherein the agent is an elastase inhibitor.

9058. The method of item 9028 wherein the agent is an elongation factor-1 alpha inhibitor.

9059. The method of item 9028 wherein the agent is an endothelial growth factor antagonist.

9060. The method of item 9028 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

9061. The method of item 9028 wherein the agent is an endotoxin antagonist.

9062. The method of item 9028 wherein the agent is an epothilone and tubulin binder.

9063. The method of item 9028 wherein the agent is an estrogen receptor antagonist.

9064. The method of item 9028 wherein the agent is an FGF inhibitor.

9065. The method of item 9028 wherein the agent is a farnexyl transferase inhibitor.

9066. The method of item 9028 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

9067. The method of item 9028 wherein the agent is an FLT-3 kinase inhibitor.

9068. The method of item 9028 wherein the agent is an FGF receptor kinase inhibitor.

9069. The method of item 9028 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

9070. The method of item 9028 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

9071. The method of item 9028 wherein the agent is a histone deacetylase inhibitor.

9072. The method of item 9028 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

9073. The method of item 9028 wherein the agent is an ICAM inhibitor.

9074. The method of item 9028 wherein the agent is an IL, ICE and IRAK antagonist, wherein the antagonist is a CJ-14877, CP-424174 (Pfizer), NF-61 (Negma-Lerads), and an analogue or derivative thereof.

9075. The method of item 9028 wherein the agent is an IL-2 inhibitor.

9076. The method of item 9028 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

9077. The method of item 9028 wherein the agent is an IMPDH (inosine monophosphate).

9078. The method of item 9028 wherein the agent is an integrin antagonist.

9079. The method of item 9028 wherein the agent is an interleukin antagonist.

9080. The method of item 9028 wherein the agent is an inhibitor of type III receptor tyrosine kinase.

9081. The method of item 9028 wherein the agent is an irreversible inhibitor of enzyme methionine aminopeptidase type 2.

9082. The method of item 9028 wherein the agent is an isozyme selective delta protein kinase C inhibitor.

9083. The method of item 9028 wherein the agent a JAK3 enzyme inhibitor.

9084. The method of item 9028 wherein the agent is a JNK inhibitor.

9085. The method of item 9028 wherein the agent is a kinase inhibitor.

9086. The method of item 9028 wherein the agent is kinesin antagonist.

9087. The method of item 9028 wherein the agent is a kinesin antagonist.

9088. The method of item 9028 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

9089. The method of item 9028 wherein the agent is an MAP kinase inhibitor.

9090. The method of item 9028 wherein the agent is a matrix metalloproteinase inhibitor.

9091. The method of item 9028 wherein the agent is an MCP-CCR2 inhibitor.

9092. The method of item 9028 wherein the agent is an mTOR inhibitor.

9093. The method of item 9028 wherein the agent is an mTOR kinase inhibitor.

9094. The method of item 9028 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

9095. The method of item 9028 wherein the agent is an MIF inhibitor.

9096. The method of item 9028 wherein the agent is an MMP inhibitor.

9097. The method of item 9028 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (GAS No. 334476-64-1) (GlaxoSmithKline), Win-64821 (Sanofi-Aventis), PRX-96026 (Predix Pharmaceuticals), and an analogue or derivative thereof.

9098. The method of item 9028 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

9099. The method of item 9028 wherein the agent is a nitric oxide agonist.

9100. The method of item 9028 wherein the agent is an ornithine decarboxylase inhibitor.

9101. The method of item 9028 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

9102. The method of item 9028 wherein the agent is a palmitoyl-protein thioesterase inhibitor.

9103. The method of item 9028 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

9104. The method of item 9028 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

9105. The method of item 9028 wherein the agent is a phosphatase inhibitor.

9106. The method of item 9028 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

9107. The method of item 9028 wherein the agent is a PKC inhibitor.

9108. The method of item 9028 wherein the agent is a platelet activating factor antagonist.

9109. The method of item 9028 wherein the agent is a platelet-derived growth factor receptor kinase inhibitor.

9110. The method of item 9028 wherein the agent is a prolyl hydroxylase inhibitor.

9111. The method of item 9028 wherein the agent is a polymorphonuclear neutrophil inhibitor.

9112. The method of item 9028 wherein the agent is a protein kinase B inhibitor.

9113. The method of item 9028 wherein the agent is a protein kinase C stimulant.

9114. The method of item 9028 wherein the agent is a purine nucleoside analogue.

9115. The method of item 9028 wherein the agent is a purinoreceptor P2X antagonist.

9116. The method of item 9028 wherein the agent is a Raf kinase inhibitor.

9117. The method of item 9028 wherein the agent is a reversible inhibitor of ErbB1 and ErbB2.

9118. The method of item 9028 wherein the agent is a ribonucleoside triphosphate reductase inhibitor.

9119. The method of item 9028 wherein the agent is an SDF-1 antagonist.

9120. The method of item 9028 wherein the agent is a sheddase inhibitor.

9121. The method of item 9028 wherein the agent is an SRC inhibitor.

9122. The method of item 9028 wherein the agent is a stromelysin inhibitor.

9123. The method of item 9028 wherein the agent is an Syk kinase inhibitor.

9124. The method of item 9028 wherein the agent is a telomerase inhibitor.

9125. The method of item 9028 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

9126. The method of item 9028 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

9127. The method of item 9028 wherein the agent is a Toll receptor inhibitor.

9128. The method of item 9028 wherein the agent is a tubulin antagonist.

9129. The method of item 9028 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

9130. The method of item 9028 wherein the agent is a VEGF inhibitor.

9131. The method of item 9028 wherein the agent is a vitamin D receptor agonist.

9132. The method of item 9028 wherein the agent is ZD-6474 (an angiogenesis inhibitor).

9133. The method of item 9028 wherein the agent is AP-23573 (an mTOR inhibitor).

9134. The method of item 9028 wherein the agent is synthadotin (a tubulin antagonist).

9135. The method of item 9028 wherein the agent is S-0885 (a collagenase inhibitor).

9136. The method of item 9028 wherein the agent is aplidine (an elongation factor-1 alpha inhibitor).

9137. The method of item 9028 wherein the agent is ixabepilone (an epithilone).

9138. The method of item 9028 wherein the agent is IDN-5390 (an angiogenesis inhibitor)

9139. The method of item 9028 wherein the agent is SB-2723005 (an angiogenesis inhibitor).

9140. The method of item 9028 wherein the agent is ABT-518 (an angiogenesis inhibitor).

9141. The method of item 9028 wherein the agent is combretastatin (an angiogenesis inhibitor).

9142. The method of item 9028 wherein the agent is anecortave acetate (an angiogenesis inhibitor).

9143. The method of item 9028 wherein the agent is SB-715992 (a kinesin antagonist).

9144. The method of item 9028 wherein the agent is temsirolimus (an mTOR inhibitor).

9145. The method of item 9028 wherein the agent is adalimumab (a TNFα antagonist).

9146. The method of item 9028, wherein the composition comprises a polymer.

9147. The method of item 9028, wherein the composition comprises a polymeric carrier.

9148. The method of item 9028 wherein the anti-scarring agent inhibits adhesion between the medical device and a host into which the medical device is implanted.

9149. The method of item 9028 wherein the medical device delivers the anti-scarring agent locally to tissue proximate to the medical device.

9150. The method of item 9028 wherein the medical device has a coating that comprises the anti-scarring agent.

9151. The method of item 9028, wherein the medical device has a coating that comprises the agent and is disposed on a surface of the electrical device.

9152. The method of item 9028, wherein the medical device has a coating that comprises the agent and directly contacts the electrical device.

9153. The method of item 9028, wherein the medical device has a coating that comprises the agent and indirectly contacts the electrical device.

9154. The method of item 9028, wherein the medical device has a coating that comprises the agent and partially covers the electrical device.

9155. The method of item 9028, wherein the medical device has a coating that comprises the agent and completely covers the electrical device.

9156. The method of item 9028, wherein the medical device has a uniform coating.

9157. The method of item 9028, wherein the medical device has a non-uniform coating.

9158. The method of item 9028, wherein the medical device has a discontinuous coating.

9159. The method of item 9028, wherein the medical device has a patterned coating.

9160. The method of item 9028, wherein the medical device has a coating with a thickness of 100 μm or less.

9161. The method of item 9028, wherein the medical device has a coating with a thickness of 10 μm or less.

9162. The method of item 9028, wherein the medical device has a coating, and the coating adheres to the surface of the electrical device upon deployment of the electrical device.

9163. The method of item 9028, wherein the medical device has a coating, and wherein the coating is stable at room temperature for a period of 1 year.

9164. The method of item 9028, wherein the medical device has a coating, and wherein the anti-scarring agent is present in the coating in an amount ranging between about 0.0001% to about 1% by weight.

9165. The method of item 9028, wherein the medical device has a coating, and wherein the anti-scarring agent is present in the coating in an amount ranging between about 1% to about 10% by weight.

9166. The method of item 9028, wherein the medical device has a coating, and wherein the anti-scarring agent is present in the coating in an amount ranging between about 10% to about 25% by weight.

9167. The method of item 9028, wherein the medical device has a coating, and wherein the anti-scarring agent is present in the coating in an amount ranging between about 25% to about 70% by weight.

9168. The method of item 9028, wherein the medical device has a coating, and wherein the coating further comprises a polymer.

9169. The method of item 9028, wherein the medical device has a first coating having a first composition and a second coating having a second composition.

9170. The method of item 9028, wherein the medical device has a first coating having a first composition and a second coating having a second composition, wherein the first composition and the second composition are different.

9171. The method of item 9028, wherein the composition comprises a polymer.

9172. The method of item 9028, wherein the composition comprises a polymeric carrier.

9173. The method of item 9028, wherein the composition comprises a polymeric carrier, and wherein the polymeric carrier comprises a copolymer.

9174. The method of item 9028, wherein the composition comprises a polymeric carrier, and wherein the polymeric carrier comprises a block copolymer.

9175. The method of item 9028, wherein the composition comprises a polymeric carrier, and wherein the polymeric carrier comprises a random copolymer.

9176. The method of item 9028, wherein the composition comprises a polymeric carrier, and wherein the polymeric carrier comprises a biodegradable polymer.

9177. The method of item 9028, wherein the composition comprises a polymeric carrier, and wherein the polymeric carrier comprises a non-biodegradable polymer.

9178. The method of item 9028, wherein the composition comprises a polymeric carrier, and wherein the polymeric carrier comprises a hydrophilic polymer.

9179. The method of item 9028, wherein the composition comprises a polymeric carrier, and wherein the polymeric carrier comprises a hydrophobic polymer.

9180. The method of item 9028, wherein the composition comprises a polymeric carrier, and wherein the polymeric carrier comprises a polymer having hydrophilic domains.

9181. The method of item 9028, wherein the composition comprises a polymeric carrier, and wherein the polymeric carrier comprises a polymer having hydrophobic domains.

9182. The method of item 9028, wherein the composition comprises a polymeric carrier, and wherein the polymeric carrier comprises a non-conductive polymer.

9183. The method of item 9028, wherein the composition comprises a polymeric carrier, and wherein the polymeric carrier comprises an elastomer.

9184. The method of item 9028, wherein the composition comprises a polymeric carrier, and wherein the polymeric carrier comprises a hydrogel.

9185. The method of item 9028, wherein the composition comprises a polymeric carrier, and wherein the polymeric carrier comprises a silicone polymer.

9186. The method of item 9028, wherein the composition comprises a polymeric carrier, and wherein the polymeric carrier comprises a hydrocarbon polymer.

9187. The method of item 9028, wherein the composition comprises a polymeric carrier, and wherein the polymeric carrier comprises a styrene-derived polymer.

9188. The method of item 9028, wherein the composition comprises a polymeric carrier, and wherein the polymeric carrier comprises a butadiene polymer.

9189. The method of item 9028, wherein the composition comprises a polymeric carrier, and wherein the polymeric carrier comprises a macromer.

9190. The method of item 9028, wherein the composition comprises a polymeric carrier, and wherein the polymeric carrier comprises a poly(ethylene glycol) polymer.

9191. The method of item 9028 wherein the composition comprises a polymeric carrier, and wherein the polymeric carrier comprises an amorphous polymer.

9192. The method of item 9028, wherein the medical device comprises a lubricious coating.

9193. The method of item 9028 wherein the anti-scarring agent is located within pores or holes of the medical device.

9194. The method of item 9028 wherein the anti-scarring agent is located within a channel, lumen, or divet of the medical device.

9195. The method of item 9028, wherein the medical device further comprises a second pharmaceutically active agent.

9196. The method of item 9028 wherein the medical device further comprises an anti-inflammatory agent.

9197. The method of item 9028 wherein the medical device further comprises an agent that inhibits infection.

9198. The method of item 9028 wherein the medical device further comprises an agent that inhibits infection, and wherein the agent is an anthracycline.

9199. The method of item 9028 wherein the medical device further comprises an agent that inhibits infection, and wherein the agent is doxorubicin.

9200. The method of item 9028 wherein the medical device further comprises an agent that inhibits infection, and wherein the agent is mitoxantrone.

9201. The method of item 9028 wherein the medical device further comprises an agent that inhibits infection, and wherein the agent is a fluoropyrimidine.

9202. The method of item 9028 wherein the medical device further comprises an agent that inhibits infection, and wherein the agent is 5-fluorouracil (5-FU).

9203. The method of item 9028 wherein the medical device further comprises an agent that inhibits infection, and wherein the agent is a folic acid antagonist.

9204. The method of item 9028 wherein the medical device further comprises an agent that inhibits infection, and wherein the agent is methotrexate.

9205. The method of item 9028 wherein the medical device further comprises an agent that inhibits infection, and wherein the agent is a podophylotoxin.

9206. The method of item 9028 wherein the medical device further comprises an agent that inhibits infection, and wherein the agent is etoposide.

9207. The method of item 9028 wherein the medical device further comprises an agent that inhibits infection, and wherein the agent is a camptothecin.

9208. The method of item 9028 wherein the medical device further comprises an agent that inhibits infection, and wherein the agent is a hydroxyurea.

9209. The method of item 9028 wherein the medical device further comprises an agent that inhibits infection, and wherein the agent is a platinum complex.

9210. The method of item 9028 wherein the medical device further comprises an agent that inhibits infection, and wherein the agent is cisplatin.

9211. The method of item 9028 wherein the medical device further comprises an anti-thrombotic agent.

9212. The method of item 9028 wherein the medical device further comprises a visualization agent.

9213. The method of item 9028 wherein the medical device further comprises a visualization agent, wherein the visualization agent is a radiopaque material, and wherein the radiopaque material further comprises a metal, a halogenated compound, or a barium containing compound.

9214. The method of item 9028 wherein the medical device further comprises a visualization agent, wherein the visualization agent is a radiopaque material, and wherein the radiopaque material further comprises barium, tantalum, or technetium.

9215. The method of item 9028 wherein the medical device further comprises a visualization agent, and wherein the visualization agent is a MRI responsive material.

9216. The method of item 9028 wherein the medical device further comprises a visualization agent, and wherein the visualization agent further comprises a gadolinium chelate

9217. The method of item 9028 wherein the medical device further comprises a visualization agent, and wherein the visualization agent further comprises iron, magnesium, manganese, copper, or chromium.

9218. The method of item 9028 wherein the medical device further comprises a visualization agent, and wherein the visualization agent further comprises an iron oxide compound.

9219. The method of item 9028 wherein the medical device further comprises a visualization agent, and wherein the visualization agent further comprises a dye, pigment, or colorant.

9220. The method of item 9028 wherein the medical device further comprises an echogenic material.

9221. The method of item 9028 wherein the medical device further comprises an echogenic material, and wherein the echogenic material is in the form of a coating.

9222. The method of item 9028 wherein the medical device is sterile.

9223. The method of item 9028 wherein the anti-scarring agent is released into tissue in the vicinity of the medical device after deployment of the medical device.

9224. The method of item 9028 wherein the anti-scarring agent is released into tissue in the vicinity of the medical device after deployment of the medical device, and wherein the tissue is connective tissue.

9225. The method of item 9028 wherein the anti-scarring agent is released into tissue in the vicinity of the medical device after deployment of the medical device, and wherein the tissue is muscle tissue.

9226. The method of item 9028 wherein the anti-scarring agent is released into tissue in the vicinity of the medical device after deployment of the medical device, and wherein the tissue is nerve tissue.

9227. The method of item 9028 wherein the anti-scarring agent is released into tissue in the vicinity of the medical device after deployment of the medical device, and wherein the tissue is epithelium tissue.

9228. The method of item 9028 wherein the anti-scarring agent is released in effective concentrations from the medical device over a period ranging from the time of deployment of the medical device to about 1 year.

9229. The method of item 9028 wherein the anti-scarring agent is released in effective concentrations from the medical device over a period ranging from about 1 month to 6 months.

9230. The method of item 9028 wherein the anti-scarring agent is released in effective concentrations from the medical device over a period ranging from about 1-90 days.

9231. The method of item 9028 wherein the anti-scarring agent is released in effective concentrations from the medical device at a constant rate.

9232. The method of item 9028 wherein the anti-scarring agent is released in effective concentrations from the medical device at an increasing rate.

9233. The method of item 9028 wherein the anti-scarring agent is released in effective concentrations from the medical device at a decreasing rate.

9234. The method of item 9028 wherein the anti-scarring agent is released in effective concentrations from the composition comprising the anti-scarring agent by diffusion over a period ranging from the time of deployment of the medical device to about 90 days.

9235. The method of item 9028 wherein the anti-scarring agent is released in effective concentrations from the composition comprising the anti-scarring agent by erosion of the composition over a period ranging from the time of deployment of the medical device to about 90 days.

9236. The method of item 9028 wherein the medical device comprises about 0.01 μg to about 10 μg of the anti-scarring agent.

9237. The method of item 9028 wherein the medical device comprises about 10 μg to about 10 mg of the anti-scarring agent.

9238. The method of item 9028 wherein the medical device comprises about 10 mg to about 250 mg of the anti-scarring agent.

9239. The method of item 9028 wherein the medical device comprises about 250 mg to about 1000 mg of the anti-scarring agent.

9240. The method of item 9028 wherein the medical device comprises about 1000 mg to about 2500 mg of the anti-scarring agent.

9241. The method of item 9028 wherein a surface of the medical device comprises less than 0.01 μg of the anti-scarring agent per mm² of medical device surface to which the anti-scarring agent is applied.

9242. The method of item 9028 wherein a surface of the medical device comprises about 0.01 μg to about 1 μg of the anti-scarring agent per mm² of medical device surface to which the anti-scarring agent is applied.

9243. The method of item 9028 wherein a surface of the medical device comprises about 1 μg to about 10 μg of the anti-scarring agent per mm² of medical device surface to which the anti-scarring agent is applied.

9244. The method of item 9028 wherein a surface of the medical device comprises about 10 μg to about 250 μg of the anti-scarring agent per mm² of medical device surface to which the anti-scarring agent is applied.

9245. The method of item 9028 wherein a surface of the medical device comprises about 250 μg to about 1000 μg of the anti-scarring agent of anti-scarring agent per mm² of medical device surface to which the anti-scarring agent is applied.

9246. The method of item 9028 wherein a surface of the medical device comprises about 1000 μg to about 2500 μg of the anti-scarring agent per mm² of medical device surface to which the anti-scarring agent is applied.

9247. The method of item 9028 wherein the combining is performed by direct affixing the agent or the composition to the electrical device.

9248. The method of item 9028 wherein the combining is performed by spraying the agent or the component onto the electrical device.

9249. The method of item 9028 wherein the combining is performed by electrospraying the agent or the composition onto the electrical device.

9250. The method of item 9028 wherein the combining is performed by dipping the electrical device into a solution comprising the agent or the composition.

9251. The method of item 9028 wherein the combining is performed by covalently attaching the agent or the composition to the electrical device.

9252. The method of item 9028 wherein the combining is performed by non-covalently attaching the agent or the composition to the electrical device.

9253. The method of item 9028 wherein the combining is performed by coating the electrical device with a substance that contains the agent or the composition.

9254. The method of item 9028 wherein the combining is performed by coating the electrical device with a substance that absorbs the agent.

9255. The method of item 9028 wherein the combining is performed by interweaving the electrical device with a thread composed of, or coated with, the agent or the composition.

9256. The method of item 9028 wherein the combining is performed by completely covering the electrical device with a sleeve that contains the agent or the composition.

9257. The method of item 9028 wherein the combining is performed by covering a portion of the electrical device with a sleeve that contains the agent or the composition.

9258. The method of item 9028 wherein the combining is performed by completely covering the electrical device with a cover that contains the agent or the composition.

9259. The method of item 9028 wherein the combining is performed by covering a portion of the electrical device with a cover that contains the agent or the composition.

9260. The method of item 9028 wherein the combining is performed by completely covering the electrical device with an electrospun fabric that contains the agent or the composition.

9261. The method of item 9028 wherein the combining is performed by covering a portion of the electrical device with an electrospun fabric that contains the agent or the composition.

9262. The method of item 9028 wherein the combining is performed by completely covering the electrical device with a mesh that contains the agent or the composition.

9263. The method of item 9028 wherein the combining is performed by covering a portion of the electrical device with a mesh that contains the agent or the composition.

9264. The method of item 9028 wherein the combining is performed by constructing a portion of the electrical device with the agent or the composition.

9265. The method of item 9028 wherein the combining is performed by impregnating the electrical device with the agent or the composition.

9266. The method of item 9028 wherein the combining is performed by constructing a portion of the electrical device from a degradable polymer that releases the agent.

9267. The method of item 9028 wherein the combining is performed by dipping the electrical device into a solution that comprise the agent and an inert solvent for the electrical device.

9268. The method of item 9028 wherein the combining is performed by dipping the electrical device into a solution that comprises the agent and a solvent that will swell the electrical device.

9269. The method of item 9028 wherein the combining is performed by dipping the electrical device into a solution that comprises the agent and a solvent that will dissolve the electrical device.

9270. The method of item 9028 wherein the combining is performed by dipping the electrical device into a solution that comprises the agent, a polymer and an inert solvent for the electrical device.

9271. The method of item 9028 wherein the combining is performed by dipping the electrical device into a solution that comprises the agent, a polymer and a solvent that will swell the electrical device.

9272. The method of item 9028 wherein the combining is performed by dipping the electrical device into a solution that comprises the agent, a polymer and a solvent that will dissolve the electrical device.

9273. The method of item 9028 wherein the combining is performed by spraying the electrical device into a solution that comprises the agent and an inert solvent for the electrical device.

9274. The method of item 9028 wherein the combining is performed by spraying the electrical device into a solution that comprises the agent and a solvent that will swell the electrical device.

9275. The method of item 9028 wherein the combining is performed by spraying the electrical device into a solution that comprises the agent and a solvent that will dissolve the electrical device.

9276. The method of item 9028 wherein the combining is performed by spraying the electrical device into a solution that comprises the agent, a polymer and an inert solvent for the electrical device.

9277. The method of item 9028 wherein the combining is performed by spraying the electrical device into a solution that comprises the agent, a polymer and a solvent that will swell the electrical device.

9278. The method of item 9028 wherein the combining is performed by spraying the electrical device into a solution that comprises the agent, a polymer and a solvent that will dissolve the electrical device.

9279. The method for making a medical device of any one of items 9028-9278 wherein the gastric nerve stimulator is adapted for treating or preventing morbid obesity.

9280. The method for making a medical device of any one of items 9028-9278 wherein the gastric nerve stimulator is adapted for treating or preventing constipation.

9281. The method for making a medical device of any one of items 9028-9278 wherein the gastric nerve stimulator comprises an electrical lead, an electrode and a stimulation generator.

9282. The method for making a medical device of any one of items 9028-9278 wherein the gastric nerve stimulator comprises an electrical signal controller, connector wire and an attachment lead.

9283. A method for making a medical device comprising: combining a cochlear implant for treating deafness (i.e., an electrical device) and an anti-scarring agent or a composition comprising an anti-scarring agent, wherein the agent inhibits scarring between the device and a host into which the device is implanted.

9284. The method of item 9283 wherein the agent is an adensosine A2A receptor antagonist.

9285. The method of item 9283 wherein the agent is an AKT inhibitor.

9286. The method of item 9283 wherein the agent is an alpha 2 integrin antagonist, wherein the alpha 2 integrin antagonist is Pharmaprojects No. 5754 (Merck KgaA).

9287. The method of item 9283 wherein the agent is an alpha 4 integrin antagonist.

9288. The method of item 9283 wherein the agent is an alpha 7 nicotinic receptor agonist.

9289. The method of item 9283 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-1 0 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

9290. The method of item 9283 wherein the agent is an apoptosis antagonist.

9291. The method of item 9283 wherein the agent is an apoptosis activator.

9292. The method of item 9283 wherein the agent is a beta 1 integrin antagonist.

9293. The method of item 9283 wherein the agent is a beta tubulin inhibitor.

9294. The method of item 9283 wherein the agent is a blocker of enzyme production in Hepatitis C.

9295. The method of item 9283 wherein the agent is a Bruton's tyrosine kinase inhibitor.

9296. The method of item 9283 wherein the agent is a calcineurin inhibitor.

9297. The method of item 9283 wherein the agent is a caspase 3 inhibitor.

9298. The method of item 9283 wherein the agent is a CC chemokine receptor antagonist.

9299. The method of item 9283 wherein the agent is a cell cycle inhibitor selected from the group consisting of SNS-595 (Sunesis), synthadotin, KRX-0403, and an analogue or derivative thereof.

9300. The method of item 9283 wherein the agent is a cathepsin B inhibitor.

9301. The method of item 9283 wherein the agent is a cathepsin K inhibitor, wherein the cathepsin K inhibitor is 462795 (GlaxoSmithKline), INPL-022-D6 (Amura Therapeutics), or an analogue or derivative thereof.

9302. The method of item 9283 wherein the agent is a cathepsin L inhibitor.

9303. The method of item 9283 wherein the agent is a CD40 antagonist.

9304. The method of item 9283 wherein the agent is a chemokine receptor agonist.

9305. The method of item 9283 wherein the agent is a chymase inhibitor.

9306. The method of item 9283 wherein the agent is a collagenase antagonist.

9307. The method of item 9283 wherein the agent is a CXCR antagonist.

9308. The method of item 9283 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

9309. The method of item 9283 wherein the agent is a cyclooxygenase 1 inhibitor.

9310. The method of item 9283 wherein the agent is a DHFR inhibitor.

9311. The method of item 9283 wherein the agent is a dual integrin inhibitor.

9312. The method of item 9283 wherein the agent is an elastase inhibitor.

9313. The method of item 9283 wherein the agent is an elongation factor-1 alpha inhibitor.

9314. The method of item 9283 wherein the agent is an endothelial growth factor antagonist.

9315. The method of item 9283 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

9316. The method of item 9283 wherein the agent is an endotoxin antagonist.

9317. The method of item 9283 wherein the agent is an epothilone and tubulin binder.

9318. The method of item 9283 wherein the agent is an estrogen receptor antagonist.

9319. The method of item 9283 wherein the agent is an FGF inhibitor.

9320. The method of item 9283 wherein the agent is a farnexyl transferase inhibitor.

9321. The method of item 9283 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

9322. The method of item 9283 wherein the agent is an FLT-3 kinase inhibitor.

9323. The method of item 9283 wherein the agent is an FGF receptor kinase inhibitor.

9324. The method of item 9283 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

9325. The method of item 9283 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

9326. The method of item 9283 wherein the agent is a histone deacetylase inhibitor.

9327. The method of item 9283 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

9328. The method of item 9283 wherein the agent is an ICAM inhibitor.

9329. The method of item 9283 wherein the agent is an IL, ICE and IRAK antagonist, wherein the antagonist is a CJ-14877, CP-424174 (Pfizer), NF-61 (Negma-Lerads), and an analogue or derivative thereof.

9330. The method of item 9283 wherein the agent is an IL-2 inhibitor.

9331. The method of item 9283 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

9332. The method of item 9283 wherein the agent is an IMPDH (inosine monophosphate).

9333. The method of item 9283 wherein the agent is an integrin antagonist.

9334. The method of item 9283 wherein the agent is an interleukin antagonist.

9335. The method of item 9283 wherein the agent is an inhibitor of type III receptor tyrosine kinase.

9336. The method of item 9283 wherein the agent is an irreversible inhibitor of enzyme methionine aminopeptidase type 2.

9337. The method of item 9283 wherein the agent is an isozyme selective delta protein kinase C inhibitor.

9338. The method of item 9283 wherein the agent a JAK3 enzyme inhibitor.

9339. The method of item 9283 wherein the agent is a JNK inhibitor.

9340. The method of item 9283 wherein the agent is a kinase inhibitor.

9341. The method of item 9283 wherein the agent is kinesin antagonist.

9342. The method of item 9283 wherein the agent is a kinesin antagonist.

9343. The method of item 9283 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

9344. The method of item 9283 wherein the agent is an MAP kinase inhibitor.

9345. The method of item 9283 wherein the agent is a matrix metalloproteinase inhibitor.

9346. The method of item 9283 wherein the agent is an MCP-CCR2 inhibitor.

9347. The method of item 9283 wherein the agent is an mTOR inhibitor.

9348. The method of item 9283 wherein the agent is an mTOR kinase inhibitor.

9349. The method of item 9283 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

9350. The method of item 9283 wherein the agent is an MIF inhibitor.

9351. The method of item 9283 wherein the agent is an MMP inhibitor.

9352. The method of item 9283 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

9353. The method of item 9283 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

9354. The method of item 9283 wherein the agent is a nitric oxide agonist.

9355. The method of item 9283 wherein the agent is an ornithine decarboxylase inhibitor.

9356. The method of item 9283 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

9357. The method of item 9283 wherein the agent is a palmitoyl-protein thioesterase inhibitor.

9358. The method of item 9283 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

9359. The method of item 9283 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

9360. The method of item 9283 wherein the agent is a phosphatase inhibitor.

9361. The method of item 9283 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

9362. The method of item 9283 wherein the agent is a PKC inhibitor.

9363. The method of item 9283 wherein the agent is a platelet activating factor antagonist.

9364. The method of item 9283 wherein the agent is a platelet-derived growth factor receptor kinase inhibitor.

9365. The method of item 9283 wherein the agent is a prolyl hydroxylase inhibitor.

9366. The method of item 9283 wherein the agent is a polymorphonuclear neutrophil inhibitor.

9367. The method of item 9283 wherein the agent is a protein kinase B inhibitor.

9368. The method of item 9283 wherein the agent is a protein kinase C stimulant.

9369. The method of item 9283 wherein the agent is a purine nucleoside analogue.

9370. The method of item 9283 wherein the agent is a purinoreceptor P2X antagonist.

9371. The method of item 9283 wherein the agent is a Raf kinase inhibitor.

9372. The method of item 9283 wherein the agent is a reversible inhibitor of ErbB1 and ErbB2.

9373. The method of item 9283 wherein the agent is a ribonucleoside triphosphate reductase inhibitor.

9374. The method of item 9283 wherein the agent is an SDF-1 antagonist.

9375. The method of item 9283 wherein the agent is a sheddase inhibitor.

9376. The method of item 9283 wherein the agent is an SRC inhibitor.

9377. The method of item 9283 wherein the agent is a stromelysin inhibitor.

9378. The method of item 9283 wherein the agent is an Syk kinase inhibitor.

9379. The method of item 9283 wherein the agent is a telomerase inhibitor.

9380. The method of item 9283 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

9381. The method of item 9283 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

9382. The method of item 9283 wherein the agent is a Toll receptor inhibitor.

9383. The method of item 9283 wherein the agent is a tubulin antagonist.

9384. The method of item 9283 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

9385. The method of item 9283 wherein the agent is a VEGF inhibitor.

9386. The method of item 9283 wherein the agent is a vitamin D receptor agonist.

9387. The method of item 9283 wherein the agent is ZD-6474 (an angiogenesis inhibitor).

9388. The method of item 9283 wherein the agent is AP-23573 (an mTOR inhibitor).

9389. The method of item 9283 wherein the agent is synthadotin (a tubulin antagonist).

9390. The method of item 9283 wherein the agent is S-0885 (a collagenase inhibitor).

9391. The method of item 9283 wherein the agent is aplidine (an elongation factor-1 alpha inhibitor).

9392. The method of item 9283 wherein the agent is ixabepilone (an epithilone).

9393. The method of item 9283 wherein the agent is IDN-5390 (an angiogenesis inhibitor).

9394. The method of item 9283 wherein the agent is SB-2723005 (an angiogenesis inhibitor).

9395. The method of item 9283 wherein the agent is ABT-518 (an angiogenesis inhibitor).

9396. The method of item 9283 wherein the agent is combretastatin (an angiogenesis inhibitor).

9397. The method of item 9283 wherein the agent is anecortave acetate (an angiogenesis inhibitor).

9398. The method of item 9283 wherein the agent is SB-715992 (a kinesin antagonist).

9399. The method of item 9283 wherein the agent is temsirolimus (an mTOR inhibitor).

9400. The method of item 9283 wherein the agent is adalimumab (a TNFα antagonist).

9401. The method of item 9283, wherein the composition comprises a polymer.

9402. The method of item 9283, wherein the composition comprises a polymeric carrier.

9403. The method of item 9283 wherein the anti-scarring agent inhibits adhesion between the medical device and a host into which the medical device is implanted.

9404. The method of item 9283 wherein the medical device delivers the anti-scarring agent locally to tissue proximate to the medical device.

9405. The method of item 9283 wherein the medical device has a coating that comprises the anti-scarring agent.

9406. The method of item 9283, wherein the medical device has a coating that comprises the agent and is disposed on a surface of the electrical device.

9407. The method of item 9283, wherein the medical device has a coating that comprises the agent and directly contacts the electrical device.

9408. The method of item 9283, wherein the medical device has a coating that comprises the agent and indirectly contacts the electrical device.

9409. The method of item 9283, wherein the medical device has a coating that comprises the agent and partially covers the electrical device.

9410. The method of item 9283, wherein the medical device has a coating that comprises the agent and completely covers the electrical device.

9411. The method of item 9283, wherein the medical device has a uniform coating.

9412. The method of item 9283, wherein the medical device has a non-uniform coating.

9413. The method of item 9283, wherein the medical device has a discontinuous coating.

9414. The method of item 9283, wherein the medical device has a patterned coating.

9415. The method of item 9283, wherein the medical device has a coating with a thickness of 100 μm or less.

9416. The method of item 9283, wherein the medical device has a coating with a thickness of 10 μm or less.

9417. The method of item 9283, wherein the medical device has a coating, and the coating adheres to the surface of the electrical device upon deployment of the electrical device.

9418. The method of item 9283, wherein the medical device has a coating, and wherein the coating is stable at room temperature for a period of 1 year.

9419. The method of item 9283, wherein the medical device has a coating, and wherein the anti-scarring agent is present in the coating in an amount ranging between about 0.0001% to about 1% by weight.

9420. The method of item 9283, wherein the medical device has a coating, and wherein the anti-scarring agent is present in the coating in an amount ranging between about 1% to about 10% by weight.

9421. The method of item 9283, wherein the medical device has a coating, and wherein the anti-scarring agent is present in the coating in an amount ranging between about 10% to about 25% by weight.

9422. The method of item 9283, wherein the medical device has a coating, and wherein the anti-scarring agent is present in the coating in an amount ranging between about 25% to about 70% by weight.

9423. The method of item 9283, wherein the medical device has a coating, and wherein the coating further comprises a polymer.

9424. The method of item 9283, wherein the medical device has a first coating having a first composition and a second coating having a second composition.

9425. The method of item 9283, wherein the medical device has a first coating having a first composition and a second coating having a second composition, wherein the first composition and the second composition are different.

9426. The method of item 9283, wherein the composition comprises a polymer.

9427. The method of item 9283, wherein the composition comprises a polymeric carrier.

9428. The method of item 9283, wherein the composition comprises a polymeric carrier, and wherein the polymeric carrier comprises a copolymer.

9429. The method of item 9283, wherein the composition comprises a polymeric carrier, and wherein the polymeric carrier comprises a block copolymer.

9430. The method of item 9283, wherein the composition comprises a polymeric carrier, and wherein the polymeric carrier comprises a random copolymer.

9431. The method of item 9283, wherein the composition comprises a polymeric carrier, and wherein the polymeric carrier comprises a biodegradable polymer.

9432. The method of item 9283, wherein the composition comprises a polymeric carrier, and wherein the polymeric carrier comprises a non-biodegradable polymer.

9433. The method of item 9283, wherein the composition comprises a polymeric carrier, and wherein the polymeric carrier comprises a hydrophilic polymer.

9434. The method of item 9283, wherein the composition comprises a polymeric carrier, and wherein the polymeric carrier comprises a hydrophobic polymer.

9435. The method of item 9283, wherein the composition comprises a polymeric carrier, and wherein the polymeric carrier comprises a polymer having hydrophilic domains.

9436. The method of item 9283, wherein the composition comprises a polymeric carrier, and wherein the polymeric carrier comprises a polymer having hydrophobic domains.

9437. The method of item 9283, wherein the composition comprises a polymeric carrier, and wherein the polymeric carrier comprises a non-conductive polymer.

9438. The method of item 9283, wherein the composition comprises a polymeric carrier, and wherein the polymeric carrier comprises an elastomer.

9439. The method of item 9283, wherein the composition comprises a polymeric carrier, and wherein the polymeric carrier comprises a hydrogel.

9440. The method of item 9283, wherein the composition comprises a polymeric carrier, and wherein the polymeric carrier comprises a silicone polymer.

9441. The method of item 9283, wherein the composition comprises a polymeric carrier, and wherein the polymeric carrier comprises a hydrocarbon polymer.

9442. The method of item 9283, wherein the composition comprises a polymeric carrier, and wherein the polymeric carrier comprises a styrene-derived polymer.

9443. The method of item 9283, wherein the composition comprises a polymeric carrier, and wherein the polymeric carrier comprises a butadiene polymer.

9444. The method of item 9283, wherein the composition comprises a polymeric carrier, and wherein the polymeric carrier comprises a macromer.

9445. The method of item 9283, wherein the composition comprises a polymeric carrier, and wherein the polymeric carrier comprises a poly(ethylene glycol) polymer.

9446. The method of item 9283 wherein the composition comprises a polymeric carrier, and wherein the polymeric carrier comprises an amorphous polymer.

9447. The method of item 9283, wherein the medical device comprises a lubricious coating.

9448. The method of item 9283 wherein the anti-scarring agent is located within pores or holes of the medical device.

9449. The method of item 9283 wherein the anti-scarring agent is located within a channel, lumen, or divet of the medical device.

9450. The method of item 9283, wherein the medical device further comprises a second pharmaceutically active agent.

9451. The method of item 9283 wherein the medical device further comprises an anti-inflammatory agent.

9452. The method of item 9283 wherein the medical device further comprises an agent that inhibits infection.

9453. The method of item 9283 wherein the medical device further comprises an agent that inhibits infection, and wherein the agent is an anthracycline.

9454. The method of item 9283 wherein the medical device further comprises an agent that inhibits infection, and wherein the agent is doxorubicin

9455. The method of item 9283 wherein the medical device further comprises an agent that inhibits infection, and wherein the agent is mitoxantrone.

9456. The method of item 9283 wherein the medical device further comprises an agent that inhibits infection, and wherein the agent is a fluoropyrimidine.

9457. The method of item 9283 wherein the medical device further comprises an agent that inhibits infection, and wherein the agent is 5-fluorouracil (5-FU).

9458. The method of item 9283 wherein the medical device further comprises an agent that inhibits infection, and wherein the agent is a folic acid antagonist.

9459. The method of item 9283 wherein the medical device further comprises an agent that inhibits infection, and wherein the agent is methotrexate.

9460. The method of item 9283 wherein the medical device further comprises an agent that inhibits infection, and wherein the agent is a podophylotoxin.

9461. The method of item 9283 wherein the medical device further comprises an agent that inhibits infection, and wherein the agent is etoposide.

9462. The method of item 9283 wherein the medical device further comprises an agent that inhibits infection, and wherein the agent is a camptothecin.

9463. The method of item 9283 wherein the medical device further comprises an agent that inhibits infection, and wherein the agent is a hydroxyurea.

9464. The method of item 9283 wherein the medical device further comprises an agent that inhibits infection, and wherein the agent is a platinum complex.

9465. The method of item 9283 wherein the medical device further comprises an agent that inhibits infection, and wherein the agent is cisplatin.

9466. The method of item 9283 wherein the medical device further comprises an anti-thrombotic agent.

9467. The method of item 9283 wherein the medical device further comprises a visualization agent.

9468. The method of item 9283 wherein the medical device further comprises a visualization agent, wherein the visualization agent is a radiopaque material, and wherein the radiopaque material further comprises a metal, a halogenated compound, or a barium containing compound.

9469. The method of item 9283 wherein the medical device further comprises a visualization agent, wherein the visualization agent is a radiopaque material, and wherein the radiopaque material further comprises barium, tantalum, or technetium.

9470. The method of item 9283 wherein the medical device further comprises a visualization agent, and wherein the visualization agent is a MRI responsive material.

9471. The method of item 9283 wherein the medical device further comprises a visualization agent, and wherein the visualization agent further comprises a gadolinium chelate.

9472. The method of item 9283 wherein the medical device further comprises a visualization agent, and wherein the visualization agent further comprises iron, magnesium, manganese, copper, or chromium.

9473. The method of item 9283 wherein the medical device further comprises a visualization agent, and wherein the visualization agent further comprises an iron oxide compound.

9474. The method of item 9283 wherein the medical device further comprises a visualization agent, and wherein the visualization agent further comprises a dye, pigment, or colorant.

9475. The method of item 9283 wherein the medical device further comprises an echogenic material.

9476. The method of item 9283 wherein the medical device further comprises an echogenic material, and wherein the echogenic material is in the form of a coating.

9477. The method of item 9283 wherein the medical device is sterile.

9478. The method of item 9283 wherein the anti-scarring agent is released into tissue in the vicinity of the medical device after deployment of the medical device.

9479. The method of item 9283 wherein the anti-scarring agent is released into tissue in the vicinity of the medical device after deployment of the medical device, and wherein the tissue is connective tissue.

9480. The method of item 9283 wherein the anti-scarring agent is released into tissue in the vicinity of the medical device after deployment of the medical device, and wherein the tissue is muscle tissue.

9481. The method of item 9283 wherein the anti-scarring agent is released into tissue in the vicinity of the medical device after deployment of the medical device, and wherein the tissue is nerve tissue.

9482. The method of item 9283 wherein the anti-scarring agent is released into tissue in the vicinity of the medical device after deployment of the medical device, and wherein the tissue is epithelium tissue.

9483. The method of item 9283 wherein the anti-scarring agent is released in effective concentrations from the medical device over a period ranging from the time of deployment of the medical device to about 1 year.

9484. The method of item 9283 wherein the anti-scarring agent is released in effective concentrations from the medical device over a period ranging from about 1 month to 6 months.

9485. The method of item 9283 wherein the anti-scarring agent is released in effective concentrations from the medical device over a period ranging from about 1-90 days.

9486. The method of item 9283 wherein the anti-scarring agent is released in effective concentrations from the medical device at a constant rate.

9487. The method of item 9283 wherein the anti-scarring agent is released in effective concentrations from the medical device at an increasing rate.

9488. The method of item 9283 wherein the anti-scarring agent is released in effective concentrations from the medical device at a decreasing rate.

9489. The method of item 9283 wherein the anti-scarring agent is released in effective concentrations from the composition comprising the anti-scarring agent by diffusion over a period ranging from the time of deployment of the medical device to about 90 days.

9490. The method of item 9283 wherein the anti-scarring agent is released in effective concentrations from the composition comprising the anti-scarring agent by erosion of the composition over a period ranging from the time of deployment of the medical device to about 90 days.

9491. The method of item 9283 wherein the medical device comprises about 0.01 μg to about 10 μg of the anti-scarring agent.

9492. The method of item 9283 wherein the medical device comprises about 10 μg to about 10 mg of the anti-scarring agent.

9493. The method of item 9283 wherein the medical device comprises about 10 mg to about 250 mg of the anti-scarring agent.

9494. The method of item 9283 wherein the medical device comprises about 250 mg to about 1000 mg of the anti-scarring agent.

9495. The method of item 9283 wherein the medical device comprises about 1000 mg to about 2500 mg of the anti-scarring agent.

9496. The method of item 9283 wherein a surface of the medical device comprises less than 0.01 μg of the anti-scarring agent per mm² of medical device surface to which the anti-scarring agent is applied.

9497. The method of item 9283 wherein a surface of the medical device comprises about 0.01 μg to about 1 μg of the anti-scarring agent per mm² of medical device surface to which the anti-scarring agent is applied.

9498. The method of item 9283 wherein a surface of the medical device comprises about 1 μg to about 10 μg of the anti-scarring agent per mm² of medical device surface to which the anti-scarring agent is applied.

9499. The method of item 9283 wherein a surface of the medical device comprises about 10 μg to about 250 μg of the anti-scarring agent per mm² of medical device surface to which the anti-scarring agent is applied.

9500. The method of item 9283 wherein a surface of the medical device comprises about 250 μg to about 1000 μg of the anti-scarring agent of anti-scarring agent per mm² of medical device surface to which the anti-scarring agent is applied.

9501. The method of item 9283 wherein a surface of the medical device comprises about 1000 μg to about 2500 μg of the anti-scarring agent per mm² of medical device surface to which the anti-scarring agent is applied.

9502. The method of item 9283 wherein the combining is performed by direct affixing the agent or the composition to the electrical device.

9503. The method of item 9283 wherein the combining is performed by spraying the agent or the component onto the electrical device.

9504. The method of item 9283 wherein the combining is performed by electrospraying the agent or the composition onto the electrical device.

9505. The method of item 9283 wherein the combining is performed by dipping the electrical device into a solution comprising the agent or the composition.

9506. The method of item 9283 wherein the combining is performed by covalently attaching the agent or the composition to the electrical device.

9507. The method of item 9283 wherein the combining is performed by non-covalently attaching the agent or the composition to the electrical device.

9508. The method of item 9283 wherein the combining is performed by coating the electrical device with a substance that contains the agent or the composition.

9509. The method of item 9283 wherein the combining is performed by coating the electrical device with a substance that absorbs the agent.

9510. The method of item 9283 wherein the combining is performed by interweaving the electrical device with a thread composed of, or coated with, the agent or the composition.

9511. The method of item 9283 wherein the combining is performed by completely covering the electrical device with a sleeve that contains the agent or the composition.

9512. The method of item 9283 wherein the combining is performed by covering a portion of the electrical device with a sleeve that contains the agent or the composition.

9513. The method of item 9283 wherein the combining is performed by completely covering the electrical device with a cover that contains the agent or the composition.

9514. The method of item 9283 wherein the combining is performed by covering a portion of the electrical device with a cover that contains the agent or the composition.

9515. The method of item 9283 wherein the combining is performed by completely covering the electrical device with an electrospun fabric that contains the agent or the composition.

9516. The method of item 9283 wherein the combining is performed by covering a portion of the electrical device with an electrospun fabric that contains the agent or the composition.

9517. The method of item 9283 wherein the combining is performed by completely covering the electrical device with a mesh that contains the agent or the composition.

9518. The method of item 9283 wherein the combining is performed by covering a portion of the electrical device with a mesh that contains the agent or the composition.

9519. The method of item 9283 wherein the combining is performed by constructing a portion of the electrical device with the agent or the composition.

9520. The method of item 9283 wherein the combining is performed by impregnating the electrical device with the agent or the composition.

9521. The method of item 9283 wherein the combining is performed by constructing a portion of the electrical device from a degradable polymer that releases the agent.

9522. The method of item 9283 wherein the combining is performed by dipping the electrical device into a solution that comprise the agent and an inert solvent for the electrical device.

9523. The method of item 9283 wherein the combining is performed by dipping the electrical device into a solution that comprises the agent and a solvent that will swell the electrical device.

9524. The method of item 9283 wherein the combining is performed by dipping the electrical device into a solution that comprises the agent and a solvent that will dissolve the electrical device.

9525. The method of item 9283 wherein the combining is performed by dipping the electrical device into a solution that comprises the agent, a polymer and an inert solvent for the electrical device.

9526. The method of item 9283 wherein the combining is performed by dipping the electrical device into a solution that comprises the agent, a polymer and a solvent that will swell the electrical device.

9527. The method of item 9283 wherein the combining is performed by dipping the electrical device into a solution that comprises the agent, a polymer and a solvent that will dissolve the electrical device.

9528. The method of item 9283 wherein the combining is performed by spraying the electrical device into a solution that comprises the agent and an inert solvent for the electrical device.

9529. The method of item 9283 wherein the combining is performed by spraying the electrical device into a solution that comprises the agent and a solvent that will swell the electrical device.

9530. The method of item 9283 wherein the combining is performed by spraying the electrical device into a solution that comprises the agent and a solvent that will dissolve the electrical device.

9531. The method of item 9283 wherein the combining is performed by spraying the electrical device into a solution that comprises the agent, a polymer and an inert solvent for the electrical device.

9532. The method of item 9283 wherein the combining is performed by spraying the electrical device into a solution that comprises the agent, a polymer and a solvent that will swell the electrical device.

9533. The method of item 9283 wherein the combining is performed by spraying the electrical device into a solution that comprises the agent, a polymer and a solvent that will dissolve the electrical device.

9534. The method for making a medical device of any one of items 9283-9533 wherein the cochlear implant comprises a plurality of transducer elements.

9535. The method for making a medical device of any one of items 9283-9533 wherein the cochlear implant comprises a sound-to-electrical stimulation encoder, a body implantable receiver-stimulator, and electrodes.

9536. The method for making a medical device of any one of items 9283-9533 wherein the cochlear implant comprises a transducer and an electrode array.

9537. The method for making a medical device of any one of items 9283-9533 wherein the cochlear implant is a subcranially implantable electromechanical system.

9538. A method for making a medical device comprising: combining a bone growth stimulator (i.e., an electrical device) and an anti-scarring agent or a composition comprising an anti-scarring agent, wherein the agent inhibits scarring between the device and a host into which the device is implanted.

9539. The method of item 9538 wherein the agent is an adensosine A2A receptor antagonist.

9540. The method of item 9538 wherein the agent is an AKT inhibitor.

9541. The method of item 9538 wherein the agent is an alpha 2 integrin antagonist, wherein the alpha 2 integrin antagonist is Pharmaprojects No. 5754 (Merck KgaA).

9542. The method of item 9538 wherein the agent is an alpha 4 integrin antagonist.

9543. The method of item 9538 wherein the agent is an alpha 7 nicotinic receptor agonist.

9544. The method of item 9538 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

9545. The method of item 9538 wherein the agent is an apoptosis antagonist.

9546. The method of item 9538 wherein the agent is an apoptosis activator.

9547. The method of item 9538 wherein the agent is a beta 1 integrin antagonist.

9548. The method of item 9538 wherein the agent is a beta tubulin inhibitor.

9549. The method of item 9538 wherein the agent is a blocker of enzyme production in Hepatitis C.

9550. The method of item 9538 wherein the agent is a Bruton's tyrosine kinase inhibitor.

9551. The method of item 9538 wherein the agent is a calcineurin inhibitor.

9552. The method of item 9538 wherein the agent is a caspase 3 inhibitor.

9553. The method of item 9538 wherein the agent is a CC chemokine receptor antagonist.

9554. The method of item 9538 wherein the agent is a cell cycle inhibitor selected from the group consisting of SNS-595 (Sunesis), synthadotin, KRX-0403, and an analogue or derivative thereof.

9555. The method of item 9538 wherein the agent is a cathepsin B inhibitor.

9556. The method of item 9538 wherein the agent is a cathepsin K inhibitor, wherein the cathepsin K inhibitor is 462795 (GlaxoSmithKline), INPL-022-D6 (Amura Therapeutics), or an analogue or derivative thereof.

9557. The method of item 9538 wherein the agent is a cathepsin L inhibitor.

9558. The method of item 9538 wherein the agent is a CD40 antagonist.

9559. The method of item 9538 wherein the agent is a chemokine receptor agonist.

9560. The method of item 9538 wherein the agent is a chymase inhibitor.

9561. The method of item 9538 wherein the agent is a collagenase antagonist.

9562. The method of item 9538 wherein the agent is a CXCR antagonist.

9563. The method of item 9538 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

9564. The method of item 9538 wherein the agent is a cyclooxygenase 1 inhibitor.

9565. The method of item 9538 wherein the agent is a DHFR inhibitor.

9566. The method of item 9538 wherein the agent is a dual integrin inhibitor.

9567. The method of item 9538 wherein the agent is an elastase inhibitor.

9568. The method of item 9538 wherein the agent is an elongation factor-1 alpha inhibitor.

9569. The method of item 9538 wherein the agent is an endothelial growth factor antagonist.

9570. The method of item 9538 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

9571. The method of item 9538 wherein the agent is an endotoxin antagonist.

9572. The method of item 9538 wherein the agent is an epothilone and tubulin binder.

9573. The method of item 9538 wherein the agent is an estrogen receptor antagonist.

9574. The method of item 9538 wherein the agent is an FGF inhibitor.

9575. The method of item 9538 wherein the agent is a farnexyl transferase inhibitor.

9576. The method of item 9538 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

9577. The method of item 9538 wherein the agent is an FLT-3 kinase inhibitor.

9578. The method of item 9538 wherein the agent is an FGF receptor kinase inhibitor.

9579. The method of item 9538 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

9580. The method of item 9538 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

9581. The method of item 9538 wherein the agent is a histone deacetylase inhibitor.

9582. The method of item 9538 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

9583. The method of item 9538 wherein the agent is an ICAM inhibitor.

9584. The method of item 9538 wherein the agent is an IL, ICE and IRAK antagonist, wherein the antagonist is a CJ-14877, CP-424174 (Pfizer), NF-61 (Negma-Lerads), and an analogue or derivative thereof.

9585. The method of item 9538 wherein the agent is an IL-2 inhibitor.

9586. The method of item 9538 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

9587. The method of item 9538 wherein the agent is an IMPDH (inosine monophosphate).

9588. The method of item 9538 wherein the agent is an integrin antagonist.

9589. The method of item 9538 wherein the agent is an interleukin antagonist.

9590. The method of item 9538 wherein the agent is an inhibitor of type III receptor tyrosine kinase.

9591. The method of item 9538 wherein the agent is an irreversible inhibitor of enzyme methionine aminopeptidase type 2.

9592. The method of item 9538 wherein the agent is an isozyme selective delta protein kinase C inhibitor.

9593. The method of item 9538 wherein the agent a JAK3 enzyme inhibitor.

9594. The method of item 9538 wherein the agent is a JNK inhibitor.

9595. The method of item 9538 wherein the agent is a kinase inhibitor.

9596. The method of item 9538 wherein the agent is kinesin antagonist.

9597. The method of item 9538 wherein the agent is a kinesin antagonist.

9598. The method of item 9538 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

9599. The method of item 9538 wherein the agent is an MAP kinase inhibitor.

9600. The method of item 9538 wherein the agent is a matrix metalloproteinase inhibitor.

9601. The method of item 9538 wherein the agent is an MCP-CCR2 inhibitor.

9602. The method of item 9538 wherein the agent is an mTOR inhibitor.

9603. The method of item 9538 wherein the agent is an mTOR kinase inhibitor.

9604. The method of item 9538 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DMI (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

9605. The method of item 9538 wherein the agent is an MIF inhibitor.

9606. The method of item 9538 wherein the agent is an MMP inhibitor.

9607. The method of item 9538 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

9608. The method of item 9538 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

9609. The method of item 9538 wherein the agent is a nitric oxide agonist.

9610. The method of item 9538 wherein the agent is an ornithine decarboxylase inhibitor.

9611. The method of item 9538 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

9612. The method of item 9538 wherein the agent is a palmitoyl-protein thioesterase inhibitor.

9613. The method of item 9538 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

9614. The method of item 9538 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

9615. The method of item 9538 wherein the agent is a phosphatase inhibitor.

9616. The method of item 9538 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

9617. The method of item 9538 wherein the agent is a PKC inhibitor.

9618. The method of item 9538 wherein the agent is a platelet activating factor antagonist.

9619. The method of item 9538 wherein the agent is a platelet-derived growth factor receptor kinase inhibitor.

9620. The method of item 9538 wherein the agent is a prolyl hydroxylase inhibitor.

9621. The method of item 9538 wherein the agent is a polymorphonuclear neutrophil inhibitor.

9622. The method of item 9538 wherein the agent is a protein kinase B inhibitor.

9623. The method of item 9538 wherein the agent is a protein kinase C stimulant.

9624. The method of item 9538 wherein the agent is a purine nucleoside analogue.

9625. The method of item 9538 wherein the agent is a purinoreceptor P2X antagonist.

9626. The method of item 9538 wherein the agent is a Raf kinase inhibitor.

9627. The method of item 9538 wherein the agent is a reversible inhibitor of ErbB1 and ErbB2.

9628. The method of item 9538 wherein the agent is a ribonucleoside triphosphate reductase inhibitor.

9629. The method of item 9538 wherein the agent is an SDF-1 antagonist.

9630. The method of item 9538 wherein the agent is a sheddase inhibitor.

9631. The method of item 9538 wherein the agent is an SRC inhibitor.

9632. The method of item 9538 wherein the agent is a stromelysin inhibitor.

9633. The method of item 9538 wherein the agent is an Syk kinase inhibitor.

9634. The method of item 9538 wherein the agent is a telomerase inhibitor.

9635. The method of item 9538 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

9636. The method of item 9538 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

9637. The method of item 9538 wherein the agent is a Toll receptor inhibitor.

9638. The method of item 9538 wherein the agent is a tubulin antagonist.

9639. The method of item 9538 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-1 3 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

9640. The method of item 9538 wherein the agent is a VEGF inhibitor.

9641. The method of item 9538 wherein the agent is a vitamin D receptor agonist.

9642. The method of item 9538 wherein the agent is ZD-6474 (an angiogenesis inhibitor).

9643. The method of item 9538 wherein the agent is AP-23573 (an mTOR inhibitor).

9644. The method of item 9538 wherein the agent is synthadotin (a tubulin antagonist).

9645. The method of item 9538 wherein the agent is S-0885 (a collagenase inhibitor).

9646. The method of item 9538 wherein the agent is aplidine (an elongation factor-1 alpha inhibitor).

9647. The method of item 9538 wherein the agent is ixabepilone (an epithilone).

9648. The method of item 9538 wherein the agent is IDN-5390 (an angiogenesis inhibitor).

9649. The method of item 9538 wherein the agent is SB-2723005 (an angiogenesis inhibitor).

9650. The method of item 9538 wherein the agent is ABT-518 (an angiogenesis inhibitor).

9651. The method of item 9538 wherein the agent is combretastatin (an angiogenesis inhibitor).

9652. The method of item 9538 wherein the agent is anecortave acetate (an angiogenesis inhibitor).

9653. The method of item 9538 wherein the agent is SB-715992 (a kinesin antagonist).

9654. The method of item 9538 wherein the agent is temsirolimus (an mTOR inhibitor).

9655. The method of item 9538 wherein the agent is adalimumab (a TNFα antagonist).

9656. The method of item 9538, wherein the composition comprises a polymer.

9657. The method of item 9538, wherein the composition comprises a polymeric carrier.

9658. The method of item 9538 wherein the anti-scarring agent inhibits adhesion between the medical device and a host into which the medical device is implanted.

9659. The method of item 9538 wherein the medical device delivers the anti-scarring agent locally to tissue proximate to the medical device.

9660. The method of item 9538 wherein the medical device has a coating that comprises the anti-scarring agent.

9661. The method of item 9538, wherein the medical device has a coating that comprises the agent and is disposed on a surface of the electrical device.

9662. The method of item 9538, wherein the medical device has a coating that comprises the agent and directly contacts the electrical device.

9663. The method of item 9538, wherein the medical device has a coating that comprises the agent and indirectly contacts the electrical device.

9664. The method of item 9538, wherein the medical device has a coating that comprises the agent and partially covers the electrical device.

9665. The method of item 9538, wherein the medical device has a coating that comprises the agent and completely covers the electrical device.

9666. The method of item 9538, wherein the medical device has a uniform coating.

9667. The method of item 9538, wherein the medical device has a non-uniform coating.

9668. The method of item 9538, wherein the medical device has a discontinuous coating.

9669. The method of item 9538, wherein the medical device has a patterned coating.

9670. The method of item 9538, wherein the medical device has a coating with a thickness of 100 μm or less.

9671. The method of item 9538, wherein the medical device has a coating with a thickness of 10 μm or less.

9672. The method of item 9538, wherein the medical device has a coating, and the coating adheres to the surface of the electrical device upon deployment of the electrical device.

9673. The method of item 9538, wherein the medical device has a coating, and wherein the coating is stable at room temperature for a period of 1 year.

9674. The method of item 9538, wherein the medical device has a coating, and wherein the anti-scarring agent is present in the coating in an amount ranging between about 0.0001% to about 1% by weight.

9675. The method of item 9538, wherein the medical device has a coating, and wherein the anti-scarring agent is present in the coating in an amount ranging between about 1% to about 10% by weight.

9676. The method of item 9538, wherein the medical device has a coating, and wherein the anti-scarring agent is present in the coating in an amount ranging between about 10% to about 25% by weight.

9677. The method of item 9538, wherein the medical device has a coating, and wherein the anti-scarring agent is present in the coating in an amount ranging between about 25% to about 70% by weight.

9678. The method of item 9538, wherein the medical device has a coating, and wherein the coating further comprises a polymer.

9679. The method of item 9538, wherein the medical device has a first coating having a first composition and a second coating having a second composition.

9680. The method of item 9538, wherein the medical device has a first coating having a first composition and a second coating having a second composition, wherein the first composition and the second composition are different.

9681. The method of item 9538, wherein the composition comprises a polymer.

9682. The method of item 9538, wherein the composition comprises a polymeric carrier.

9683. The method of item 9538, wherein the composition comprises a polymeric carrier, and wherein the polymeric carrier comprises a copolymer.

9684. The method of item 9538, wherein the composition comprises a polymeric carrier, and wherein the polymeric carrier comprises a block copolymer.

9685. The method of item 9538, wherein the composition comprises a polymeric carrier, and wherein the polymeric carrier comprises a random copolymer.

9686. The method of item 9538, wherein the composition comprises a polymeric carrier, and wherein the polymeric carrier comprises a biodegradable polymer.

9687. The method of item 9538, wherein the composition comprises a polymeric carrier, and wherein the polymeric carrier comprises a non-biodegradable polymer.

9688. The method of item 9538, wherein the composition comprises a polymeric carrier, and wherein the polymeric carrier comprises a hydrophilic polymer.

9689. The method of item 9538, wherein the composition comprises a polymeric carrier, and wherein the polymeric carrier comprises a hydrophobic polymer.

9690. The method of item 9538, wherein the composition comprises a polymeric carrier, and wherein the polymeric carrier comprises a polymer having hydrophilic domains.

9691. The method of item 9538, wherein the composition comprises a polymeric carrier, and wherein the polymeric carrier comprises a polymer having hydrophobic domains.

9692. The method of item 9538, wherein the composition comprises a polymeric carrier, and wherein the polymeric carrier comprises a non-conductive polymer.

9693. The method of item 9538, wherein the composition comprises a polymeric carrier, and wherein the polymeric carrier comprises an elastomer.

9694. The method of item 9538, wherein the composition comprises a polymeric carrier, and wherein the polymeric carrier comprises a hydrogel.

9695. The method of item 9538, wherein the composition comprises a polymeric carrier, and wherein the polymeric carrier comprises a silicone polymer.

9696. The method of item 9538, wherein the composition comprises a polymeric carrier, and wherein the polymeric carrier comprises a hydrocarbon polymer.

9697. The method of item 9538, wherein the composition comprises a polymeric carrier, and wherein the polymeric carrier comprises a styrene-derived polymer.

9698. The method of item 9538, wherein the composition comprises a polymeric carrier, and wherein the polymeric carrier comprises a butadiene polymer.

9699. The method of item 9538, wherein the composition comprises a polymeric carrier, and wherein the polymeric carrier comprises a macromer.

9700. The method of item 9538, wherein the composition comprises a polymeric carrier, and wherein the polymeric carrier comprises a poly(ethylene glycol) polymer.

9701. The method of item 9538 wherein the composition comprises a polymeric carrier, and wherein the polymeric carrier comprises an amorphous polymer.

9702. The method of item 9538, wherein the medical device comprises a lubricious coating.

9703. The method of item 9538 wherein the anti-scarring agent is located within pores or holes of the medical device.

9704. The method of item 9538 wherein the anti-scarring agent is located within a channel, lumen, or divet of the medical device.

9705. The method of item 9538, wherein the medical device further comprises a second pharmaceutically active agent.

9706. The method of item 9538 wherein the medical device further comprises an anti-inflammatory agent.

9707. The method of item 9538 wherein the medical device further comprises an agent that inhibits infection.

9708. The method of item 9538 wherein the medical device further comprises an agent that inhibits infection, and wherein the agent is an anthracycline.

9709. The method of item 9538 wherein the medical device further comprises an agent that inhibits infection, and wherein the agent is doxorubicin.

9710. The method of item 9538 wherein the medical device further comprises an agent that inhibits infection, and wherein the agent is mitoxantrone.

9711. The method of item 9538 wherein the medical device further comprises an agent that inhibits infection, and wherein the agent is a fluoropyrimidine.

9712. The method of item 9538 wherein the medical device further comprises an agent that inhibits infection, and wherein the agent is 5-fluorouracil (5-FU).

9713. The method of item 9538 wherein the medical device further comprises an agent that inhibits infection, and wherein the agent is a folic acid antagonist.

9714. The method of item 9538 wherein the medical device further comprises an agent that inhibits infection, and wherein the agent is methotrexate.

9715. The method of item 9538 wherein the medical device further comprises an agent that inhibits infection, and wherein the agent is a podophylotoxin.

9716. The method of item 9538 wherein the medical device further comprises an agent that inhibits infection, and wherein the agent is etoposide.

9717. The method of item 9538 wherein the medical device further comprises an agent that inhibits infection, and wherein the agent is a camptothecin.

9718. The method of item 9538 wherein the medical device further comprises an agent that inhibits infection, and wherein the agent is a hydroxyurea.

9719. The method of item 9538 wherein the medical device further comprises an agent that inhibits infection, and wherein the agent is a platinum complex.

9720. The method of item 9538 wherein the medical device further comprises an agent that inhibits infection, and wherein the agent is cisplatin.

9721. The method of item 9538 wherein the medical device further comprises an anti-thrombotic agent.

9722. The method of item 9538 wherein the medical device further comprises a visualization agent.

9723. The method of item 9538 wherein the medical device further comprises a visualization agent, wherein the visualization agent is a radiopaque material, and wherein the radiopaque material further comprises a metal, a halogenated compound, or a barium containing compound.

9724. The method of item 9538 wherein the medical device further comprises a visualization agent, wherein the visualization agent is a radiopaque material, and wherein the radiopaque material further comprises barium, tantalum, or technetium.

9725. The method of item 9538 wherein the medical device further comprises a visualization agent, and wherein the visualization agent is a MRI responsive material.

9726. The method of item 9538 wherein the medical device further comprises a visualization agent, and wherein the visualization agent further comprises a gadolinium chelate.

9727. The method of item 9538 wherein the medical device further comprises a visualization agent, and wherein the visualization agent further comprises iron, magnesium, manganese, copper, or chromium.

9728. The method of item 9538 wherein the medical device further comprises a visualization agent, and wherein the visualization agent further comprises an iron oxide compound.

9729. The method of item 9538 wherein the medical device further comprises a visualization agent, and wherein the visualization agent further comprises a dye, pigment, or colorant.

9730. The method of item 9538 wherein the medical device further comprises an echogenic material.

9731. The method of item 9538 wherein the medical device further comprises an echogenic material, and wherein the echogenic material is in the form of a coating.

9732. The method of item 9538 wherein the medical device is sterile.

9733. The method of item 9538 wherein the anti-scarring agent is released into tissue in the vicinity of the medical device after deployment of the medical device.

9734. The method of item 9538 wherein the anti-scarring agent is released into tissue in the vicinity of the medical device after deployment of the medical device, and wherein the tissue is connective tissue

9735. The method of item 9538 wherein the anti-scarring agent is released into tissue in the vicinity of the medical device after deployment of the medical device, and wherein the tissue is muscle tissue.

9736. The method of item 9538 wherein the anti-scarring agent is released into tissue in the vicinity of the medical device after deployment of the medical device, and wherein the tissue is nerve tissue

9737. The method of item 9538 wherein the anti-scarring agent is released into tissue in the vicinity of the medical device after deployment of the medical device, and wherein the tissue is epithelium tissue

9738. The method of item 9538 wherein the anti-scarring agent is released in effective concentrations from the medical device over a period ranging from the time of deployment of the medical device to about 1 year.

9739. The method of item 9538 wherein the anti-scarring agent is released in effective concentrations from the medical device over a period ranging from about 1 month to 6 months.

9740. The method of item 9538 wherein the anti-scarring agent is released in effective concentrations from the medical device over a period ranging from about 1-90 days.

9741. The method of item 9538 wherein the anti-scarring agent is released in effective concentrations from the medical device at a constant rate.

9742. The method of item 9538 wherein the anti-scarring agent is released in effective concentrations from the medical device at an increasing rate.

9743. The method of item 9538 wherein the anti-scarring agent is released in effective concentrations from the medical device at a decreasing rate.

9744. The method of item 9538 wherein the anti-scarring agent is released in effective concentrations from the composition comprising the anti-scarring agent by diffusion over a period ranging from the time of deployment of the medical device to about 90 days.

9745. The method of item 9538 wherein the anti-scarring agent is released in effective concentrations from the composition comprising the anti-scarring agent by erosion of the composition over a period ranging from the time of deployment of the medical device to about 90 days.

9746. The method of item 9538 wherein the medical device comprises about 0.01 μg to about 10 μg of the anti-scarring agent.

9747. The method of item 9538 wherein the medical device comprises about 10 μg to about 10 mg of the anti-scarring agent.

9748. The method of item 9538 wherein the medical device comprises about 10 mg to about 250 mg of the anti-scarring agent.

9749. The method of item 9538 wherein the medical device comprises about 250 mg to about 1000 mg of the anti-scarring agent.

9750. The method of item 9538 wherein the medical device comprises about 1000 mg to about 2500 mg of the anti-scarring agent.

9751. The method of item 9538 wherein a surface of the medical device comprises less than 0.01 μg of the anti-scarring agent per mm² of medical device surface to which the anti-scarring agent is applied.

9752. The method of item 9538 wherein a surface of the medical device comprises about 0.01 μg to about 1 μg of the anti-scarring agent per mm² of medical device surface to which the anti-scarring agent is applied.

9753. The method of item 9538 wherein a surface of the medical device comprises about 1 μg to about 10 μg of the anti-scarring agent per mm² of medical device surface to which the anti-scarring agent is applied.

9754. The method of item 9538 wherein a surface of the medical device comprises about 10 μg to about 250 μg of the anti-scarring agent per mm² of medical device surface to which the anti-scarring agent is applied.

9755. The method of item 9538 wherein a surface of the medical device comprises about 250 μg to about 1000 μg of the anti-scarring agent of anti-scarring agent per mm² of medical device surface to which the anti-scarring agent is applied.

9756. The method of item 9538 wherein a surface of the medical device comprises about 1000 μg to about 2500 μg of the anti-scarring agent per mm² of medical device surface to which the anti-scarring agent is applied.

9757. The method of item 9538 wherein the combining is performed by direct affixing the agent or the composition to the electrical device.

9758. The method of item 9538 wherein the combining is performed by spraying the agent or the component onto the electrical device.

9759. The method of item 9538 wherein the combining is performed by electrospraying the agent or the composition onto the electrical device.

9760. The method of item 9538 wherein the combining is performed by dipping the electrical device into a solution comprising the agent or the composition.

9761. The method of item 9538 wherein the combining is performed by covalently attaching the agent or the composition to the electrical device.

9762. The method of item 9538 wherein the combining is performed by non-covalently attaching the agent or the composition to the electrical device.

9763. The method of item 9538 wherein the combining is performed by coating the electrical device with a substance that contains the agent or the composition.

9764. The method of item 9538 wherein the combining is performed by coating the electrical device with a substance that absorbs the agent.

9765. The method of item 9538 wherein the combining is performed by interweaving the electrical device with a thread composed of, or coated with, the agent or the composition.

9766. The method of item 9538 wherein the combining is performed by completely covering the electrical device with a sleeve that contains the agent or the composition.

9767. The method of item 9538 wherein the combining is performed by covering a portion of the electrical device with a sleeve that contains the agent or the composition.

9768. The method of item 9538 wherein the combining is performed by completely covering the electrical device with a cover that contains the agent or the composition.

9769. The method of item 9538 wherein the combining is performed by covering a portion of the electrical device with a cover that contains the agent or the composition.

9770. The method of item 9538 wherein the combining is performed by completely covering the electrical device with an electrospun fabric that contains the agent or the composition.

9771. The method of item 9538 wherein the combining is performed by covering a portion of the electrical device with an electrospun fabric that contains the agent or the composition.

9772. The method of item 9538 wherein the combining is performed by completely covering the electrical device with a mesh that contains the agent or the composition.

9773. The method of item 9538 wherein the combining is performed by covering a portion of the electrical device with a mesh that contains the agent or the composition.

9774. The method of item 9538 wherein the combining is performed by constructing a portion of the electrical device with the agent or the composition.

9775. The method of item 9538 wherein the combining is performed by impregnating the electrical device with the agent or the composition.

9776. The method of item 9538 wherein the combining is performed by constructing a portion of the electrical device from a degradable polymer that releases the agent.

9777. The method of item 9538 wherein the combining is performed by dipping the electrical device into a solution that comprise the agent and an inert solvent for the electrical device.

9778. The method of item 9538 wherein the combining is performed by dipping the electrical device into a solution that comprises the agent and a solvent that will swell the electrical device.

9779. The method of item 9538 wherein the combining is performed by dipping the electrical device into a solution that comprises the agent and a solvent that will dissolve the electrical device.

9780. The method of item 9538 wherein the combining is performed by dipping the electrical device into a solution that comprises the agent, a polymer and an inert solvent for the electrical device.

9781. The method of item 9538 wherein the combining is performed by dipping the electrical device into a solution that comprises the agent, a polymer and a solvent that will swell the electrical device.

9782. The method of item 9538 wherein the combining is performed by dipping the electrical device into a solution that comprises the agent, a polymer and a solvent that will dissolve the electrical device.

9783. The method of item 9538 wherein the combining is performed by spraying the electrical device into a solution that comprises the agent and an inert solvent for the electrical device.

9784. The method of item 9538 wherein the combining is performed by spraying the electrical device into a solution that comprises the agent and a solvent that will swell the electrical device.

9785. The method of item 9538 wherein the combining is performed by spraying the electrical device into a solution that comprises the agent and a solvent that will dissolve the electrical device.

9786. The method of item 9538 wherein the combining is performed by spraying the electrical device into a solution that comprises the agent, a polymer and an inert solvent for the electrical device.

9787. The method of item 9538 wherein the combining is performed by spraying the electrical device into a solution that comprises the agent, a polymer and a solvent that will swell the electrical device.

9788. The method of item 9538 wherein the combining is performed by spraying the electrical device into a solution that comprises the agent, a polymer and a solvent that will dissolve the electrical device.

9789. The method for making a medical device of any one of items 9538-9788 wherein the bone growth stimulator comprises an electrode and a generator having a strain response piezoelectric material that responds to strain.

9790. A method for making a medical device comprising: combining a cardiac pacemaker (i.e., an electrical device) and an anti-scarring agent or a composition comprising an anti-scarring agent, wherein the agent inhibits scarring between the device and a host into which the device is implanted.

9791. The method of item 9790 wherein the agent is an adensosine A2A receptor antagonist.

9792. The method of item 9790 wherein the agent is an AKT inhibitor.

9793. The method of item 9790 wherein the agent is an alpha 2 integrin antagonist, wherein the alpha 2 integrin antagonist is Pharmaprojects No. 5754 (Merck KgaA).

9794. The method of item 9790 wherein the agent is an alpha 4 integrin antagonist.

9795. The method of item 9790 wherein the agent is an alpha 7 nicotinic receptor agonist.

9796. The method of item 9790 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-1 6 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

9797. The method of item 9790 wherein the agent is an apoptosis antagonist.

9798. The method of item 9790 wherein the agent is an apoptosis activator.

9799. The method of item 9790 wherein the agent is a beta 1 integrin antagonist.

9800. The method of item 9790 wherein the agent is a beta tubulin inhibitor.

9801. The method of item 9790 wherein the agent is a blocker of enzyme production in Hepatitis C.

9802. The method of item 9790 wherein the agent is a Bruton's tyrosine kinase inhibitor.

9803. The method of item 9790 wherein the agent is a calcineurin inhibitor.

9804. The method of item 9790 wherein the agent is a caspase 3 inhibitor.

9805. The method of item 9790 wherein the agent is a CC chemokine receptor antagonist.

9806. The method of item 9790 wherein the agent is a cell cycle inhibitor selected from the group consisting of SNS-595 (Sunesis), synthadotin, KRX-0403, and an analogue or derivative thereof.

9807. The method of item 9790 wherein the agent is a cathepsin B inhibitor.

9808. The method of item 9790 wherein the agent is a cathepsin K inhibitor, wherein the cathepsin K inhibitor is 462795 (GlaxoSmithKline), INPL-022-D6 (Amura Therapeutics), or an analogue or derivative thereof.

9809. The method of item 9790 wherein the agent is a cathepsin L inhibitor.

9810. The method of item 9790 wherein the agent is a CD40 antagonist.

9811. The method of item 9790 wherein the agent is a chemokine receptor agonist.

9812. The method of item 9790 wherein the agent is a chymase inhibitor.

9813. The method of item 9790 wherein the agent is a collagenase antagonist.

9814. The method of item 9790 wherein the agent is a CXCR antagonist.

9815. The method of item 9790 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

9816. The method of item 9790 wherein the agent is a cyclooxygenase 1 inhibitor.

9817. The method of item 9790 wherein the agent is a DHFR inhibitor.

9818. The method of item 9790 wherein the agent is a dual integrin inhibitor.

9819. The method of item 9790 wherein the agent is an elastase inhibitor.

9820. The method of item 9790 wherein the agent is an elongation factor-1 alpha inhibitor.

9821. The method of item 9790 wherein the agent is an endothelial growth factor antagonist.

9822. The method of item 9790 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

9823. The method of item 9790 wherein the agent is an endotoxin antagonist.

9824. The method of item 9790 wherein the agent is an epothilone and tubulin binder.

9825. The method of item 9790 wherein the agent is an estrogen receptor antagonist.

9826. The method of item 9790 wherein the agent is an FGF inhibitor.

9827. The method of item 9790 wherein the agent is a farnexyl transferase inhibitor.

9828. The method of item 9790 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof

9829. The method of item 9790 wherein the agent is an FLT-3 kinase inhibitor.

9830. The method of item 9790 wherein the agent is an FGF receptor kinase inhibitor.

9831. The method of item 9790 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

9832. The method of item 9790 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

9833. The method of item 9790 wherein the agent is a histone deacetylase inhibitor.

9834. The method of item 9790 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

9835. The method of item 9790 wherein the agent is an ICAM inhibitor.

9836. The method of item 9790 wherein the agent is an IL, ICE and IRAK antagonist, wherein the antagonist is a CJ-14877, CP-424174 (Pfizer), NF-61 (Negma-Lerads), and an analogue or derivative thereof.

9837. The method of item 9790 wherein the agent is an IL-2 inhibitor.

9838. The method of item 9790 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

9839. The method of item 9790 wherein the agent is an IMPDH (inosine monophosphate).

9840. The method of item 9790 wherein the agent is an integrin antagonist.

9841. The method of item 9790 wherein the agent is an interleukin antagonist.

9842. The method of item 9790 wherein the agent is an inhibitor of type III receptor tyrosine kinase.

9843. The method of item 9790 wherein the agent is an irreversible inhibitor of enzyme methionine aminopeptidase type 2.

9844. The method of item 9790 wherein the agent is an isozyme selective delta protein kinase C inhibitor.

9845. The method of item 9790 wherein the agent a JAK3 enzyme inhibitor.

9846. The method of item 9790 wherein the agent is a JNK inhibitor.

9847. The method of item 9790 wherein the agent is a kinase inhibitor.

9848. The method of item 9790 wherein the agent is kinesin antagonist.

9849. The method of item 9790 wherein the agent is a kinesin antagonist.

9850. The method of item 9790 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 1119477-85-9) (Pfizer), and analogue or derivative thereof.

9851. The method of item 9790 wherein the agent is an MAP kinase inhibitor.

9852. The method of item 9790 wherein the agent is a matrix metalloproteinase inhibitor.

9853. The method of item 9790 wherein the agent is an MCP-CCR2 inhibitor.

9854. The method of item 9790 wherein the agent is an mTOR inhibitor.

9855. The method of item 9790 wherein the agent is an mTOR kinase inhibitor.

9856. The method of item 9790 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

9857. The method of item 9790 wherein the agent is an MIF inhibitor.

9858. The method of item 9790 wherein the agent is an MMP inhibitor.

9859. The method of item 9790 wherein the agent is a neurokinin (NK) antagonist selected from the group consisting of anthrotainin (CAS No. 14808440-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

9860. The method of item 9790 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

9861. The method of item 9790 wherein the agent is a nitric oxide agonist.

9862. The method of item 9790 wherein the agent is an ornithine decarboxylase inhibitor.

9863. The method of item 9790 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

9864. The method of item 9790 wherein the agent is a palmitoyl-protein thioesterase inhibitor.

9865. The method of item 9790 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

9866. The method of item 9790 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

9867. The method of item 9790 wherein the agent is a phosphatase inhibitor.

9868. The method of item 9790 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

9869. The method of item 9790 wherein the agent is a PKC inhibitor.

9870. The method of item 9790 wherein the agent is a platelet activating factor antagonist.

9871. The method of item 9790 wherein the agent is a platelet-derived growth factor receptor kinase inhibitor.

9872. The method of item 9790 wherein the agent is a prolyl hydroxylase inhibitor.

9873. The method of item 9790 wherein the agent is a polymorphonuclear neutrophil inhibitor.

9874. The method of item 9790 wherein the agent is a protein kinase B inhibitor.

9875. The method of item 9790 wherein the agent is a protein kinase C stimulant.

9876. The method of item 9790 wherein the agent is a purine nucleoside analogue.

9877. The method of item 9790 wherein the agent is a purinoreceptor P2X antagonist.

9878. The method of item 9790 wherein the agent is a Raf kinase inhibitor.

9879. The method of item 9790 wherein the agent is a reversible inhibitor of ErbB1 and ErbB2.

9880. The method of item 9790 wherein the agent is a ribonucleoside triphosphate reductase inhibitor.

9881. The method of item 9790 wherein the agent is an SDF-1 antagonist.

9882. The method of item 9790 wherein the agent is a sheddase inhibitor.

9883. The method of item 9790 wherein the agent is an SRC inhibitor.

9884. The method of item 9790 wherein the agent is a stromelysin inhibitor.

9885. The method of item 9790 wherein the agent is an Syk kinase inhibitor.

9886. The method of item 9790 wherein the agent is a telomerase inhibitor.

9887. The method of item 9790 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

9888. The method of item 9790 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

9889. The method of item 9790 wherein the agent is a Toll receptor inhibitor.

9890. The method of item 9790 wherein the agent is a tubulin antagonist.

9891. The method of item 9790 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (GAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

9892. The method of item 9790 wherein the agent is a VEGF inhibitor.

9893. The method of item 9790 wherein the agent is a vitamin D receptor agonist.

9894. The method of item 9790 wherein the agent is ZD-6474 (an angiogenesis inhibitor).

9895. The method of item 9790 wherein the agent is AP-23573 (an mTOR inhibitor).

9896. The method of item 9790 wherein the agent is synthadotin (a tubulin antagonist).

9897. The method of item 9790 wherein the agent is S-0885 (a collagenase inhibitor).

9898. The method of item 9790 wherein the agent is aplidine (an elongation factor-1 alpha inhibitor).

9899. The method of item 9790 wherein the agent is ixabepilone (an epithilone).

9900. The method of item 9790 wherein the agent is IDN-5390 (an angiogenesis inhibitor).

9901. The method of item 9790 wherein the agent is SB-2723005 (an angiogenesis inhibitor).

9902. The method of item 9790 wherein the agent is ABT-518 (an angiogenesis inhibitor).

9903. The method of item 9790 wherein the agent is combretastatin (an angiogenesis inhibitor).

9904. The method of item 9790 wherein the agent is anecortave acetate (an angiogenesis inhibitor).

9905. The method of item 9790 wherein the agent is SB-715992 (a kinesin antagonist).

9906. The method of item 9790 wherein the agent is temsirolimus (an mTOR inhibitor).

9907. The method of item 9790 wherein the agent is adalimumab (a TNFα antagonist).

9908. The method of item 9790, wherein the composition comprises a polymer.

9909. The method of item 9790, wherein the composition comprises a polymeric carrier.

9910. The method of item 9790 wherein the anti-scarring agent inhibits adhesion between the medical device and a host into which the medical device is implanted.

9911. The method of item 9790 wherein the medical device delivers the anti-scarring agent locally to tissue proximate to the medical device.

9912. The method of item 9790 wherein the medical device has a coating that comprises the anti-scarring agent.

9913. The method of item 9790, wherein the medical device has a coating that comprises the agent and is disposed on a surface of the electrical device.

9914. The method of item 9790, wherein the medical device has a coating that comprises the agent and directly contacts the electrical device.

9915. The method of item 9790, wherein the medical device has a coating that comprises the agent and indirectly contacts the electrical device.

9916. The method of item 9790, wherein the medical device has a coating that comprises the agent and partially covers the electrical device.

9917. The method of item 9790, wherein the medical device has a coating that comprises the agent and completely covers the electrical device.

9918. The method of item 9790, wherein the medical device has a uniform coating.

9919. The method of item 9790, wherein the medical device has a non-uniform coating.

9920. The method of item 9790, wherein the medical device has a discontinuous coating.

9921. The method of item 9790, wherein the medical device has a patterned coating.

9922. The method of item 9790, wherein the medical device has a coating with a thickness of 100 μm or less.

9923. The method of item 9790, wherein the medical device has a coating with a thickness of 10 μm or less.

9924. The method of item 9790, wherein the medical device has a coating, and the coating adheres to the surface of the electrical device upon deployment of the electrical device.

9925. The method of item 9790, wherein the medical device has a coating, and wherein the coating is stable at room temperature for a period of 1 year.

9926. The method of item 9790, wherein the medical device has a coating, and wherein the anti-scarring agent is present in the coating in an amount ranging between about 0.0001% to about 1% by weight.

9927. The method of item 9790, wherein the medical device has a coating, and wherein the anti-scarring agent is present in the coating in an amount ranging between about 1% to about 10% by weight.

9928. The method of item 9790, wherein the medical device has a coating, and wherein the anti-scarring agent is present in the coating in an amount ranging between about 10% to about 25% by weight.

9929. The method of item 9790, wherein the medical device has a coating, and wherein the anti-scarring agent is present in the coating in an amount ranging between about 25% to about 70% by weight.

9930. The method of item 9790, wherein the medical device has a coating, and wherein the coating further comprises a polymer.

9931. The method of item 9790, wherein the medical device has a first coating having a first composition and a second coating having a second composition.

9932. The method of item 9790, wherein the medical device has a first coating having a first composition and a second coating having a second composition, wherein the first composition and the second composition are different.

9933. The method of item 9790, wherein the composition comprises a polymer.

9934. The method of item 9790, wherein the composition comprises a polymeric carrier.

9935. The method of item 9790, wherein the composition comprises a polymeric carrier, and wherein the polymeric carrier comprises a copolymer.

9936. The method of item 9790, wherein the composition comprises a polymeric carrier, and wherein the polymeric carrier comprises a block copolymer.

9937. The method of item 9790, wherein the composition comprises a polymeric carrier, and wherein the polymeric carrier comprises a random copolymer.

9938. The method of item 9790, wherein the composition comprises a polymeric carrier, and wherein the polymeric carrier comprises a biodegradable polymer.

9939. The method of item 9790, wherein the composition comprises a polymeric carrier, and wherein the polymeric carrier comprises a non-biodegradable polymer.

9940. The method of item 9790, wherein the composition comprises a polymeric carrier, and wherein the polymeric carrier comprises a hydrophilic polymer.

9941. The method of item 9790, wherein the composition comprises a polymeric carrier, and wherein the polymeric carrier comprises a hydrophobic polymer.

9942. The method of item 9790, wherein the composition comprises a polymeric carrier, and wherein the polymeric carrier comprises a polymer having hydrophilic domains.

9943. The method of item 9790, wherein the composition comprises a polymeric carrier, and wherein the polymeric carrier comprises a polymer having hydrophobic domains.

9944. The method of item 9790, wherein the composition comprises a polymeric carrier, and wherein the polymeric carrier comprises a non-conductive polymer.

9945. The method of item 9790, wherein the composition comprises a polymeric carrier, and wherein the polymeric carrier comprises an elastomer.

9946. The method of item 9790, wherein the composition comprises a polymeric carrier, and wherein the polymeric carrier comprises a hydrogel.

9947. The method of item 9790, wherein the composition comprises a polymeric carrier, and wherein the polymeric carrier comprises a silicone polymer.

9948. The method of item 9790, wherein the composition comprises a polymeric carrier, and wherein the polymeric carrier comprises a hydrocarbon polymer.

9949. The method of item 9790, wherein the composition comprises a polymeric carrier, and wherein the polymeric carrier comprises a styrene-derived polymer.

9950. The method of item 9790, wherein the composition comprises a polymeric carrier, and wherein the polymeric carrier comprises a butadiene polymer.

9951. The method of item 9790, wherein the composition comprises a polymeric carrier, and wherein the polymeric carrier comprises a macromer.

9952. The method of item 9790, wherein the composition comprises a polymeric carrier, and wherein the polymeric carrier comprises a poly(ethylene glycol) polymer.

9953. The method of item 9790 wherein the composition comprises a polymeric carrier, and wherein the polymeric carrier comprises an amorphous polymer.

9954. The method of item 9790, wherein the medical device comprises a lubricious coating.

9955. The method of item 9790 wherein the anti-scarring agent is located within pores or holes of the medical device.

9956. The method of item 9790 wherein the anti-scarring agent is located within a channel, lumen, or divet of the medical device.

9957. The method of item 9790, wherein the medical device further comprises a second pharmaceutically active agent.

9958. The method of item 9790 wherein the medical device further comprises an anti-inflammatory agent.

9959. The method of item 9790 wherein the medical device further comprises an agent that inhibits infection.

9960. The method of item 9790 wherein the medical device further comprises an agent that inhibits infection, and wherein the agent is an anthracycline.

9961. The method of item 9790 wherein the medical device further comprises an agent that inhibits infection, and wherein the agent is doxorubicin.

9962. The method of item 9790 wherein the medical device further comprises an agent that inhibits infection, and wherein the agent is mitoxantrone.

9963. The method of item 9790 wherein the medical device further comprises an agent that inhibits infection, and wherein the agent is a fluoropyrimidine.

9964. The method of item 9790 wherein the medical device further comprises an agent that inhibits infection, and wherein the agent is 5-fluorouracil (5-FU).

9965. The method of item 9790 wherein the medical device further comprises an agent that inhibits infection, and wherein the agent is a folic acid antagonist.

9966. The method of item 9790 wherein the medical device further comprises an agent that inhibits infection, and wherein the agent is methotrexate.

9967. The method of item 9790 wherein the medical device further comprises an agent that inhibits infection, and wherein the agent is a podophylotoxin.

9968. The method of item 9790 wherein the medical device further comprises an agent that inhibits infection, and wherein the agent is etoposide.

9969. The method of item 9790 wherein the medical device further comprises an agent that inhibits infection, and wherein the agent is a camptothecin.

9970. The method of item 9790 wherein the medical device further comprises an agent that inhibits infection, and wherein the agent is a hydroxyurea.

9971. The method of item 9790 wherein the medical device further comprises an agent that inhibits infection, and wherein the agent is a platinum complex.

9972. The method of item 9790 wherein the medical device further comprises an agent that inhibits infection, and wherein the agent is cisplatin.

9973. The method of item 9790 wherein the medical device further comprises an anti-thrombotic agent.

9974. The method of item 9790 wherein the medical device further comprises a visualization agent.

9975. The method of item 9790 wherein the medical device further comprises a visualization agent, wherein the visualization agent is a radiopaque material, and wherein the radiopaque material further comprises a metal, a halogenated compound, or a barium containing compound.

9976. The method of item 9790 wherein the medical device further comprises a visualization agent, wherein the visualization agent is a radiopaque material, and wherein the radiopaque material further comprises barium, tantalum, or technetium.

9977. The method of item 9790 wherein the medical device further comprises a visualization agent, and wherein the visualization agent is a MRI responsive material.

9978. The method of item 9790 wherein the medical device further comprises a visualization agent, and wherein the visualization agent further comprises a gadolinium chelate.

9979. The method of item 9790 wherein the medical device further comprises a visualization agent, and wherein the visualization agent further comprises iron, magnesium, manganese, copper, or chromium.

9980. The method of item 9790 wherein the medical device further comprises a visualization agent, and wherein the visualization agent further comprises an iron oxide compound.

9981. The method of item 9790 wherein the medical device further comprises a visualization agent, and wherein the visualization agent further comprises a dye, pigment, or colorant.

9982. The method of item 9790 wherein the medical device further comprises an echogenic material.

9983. The method of item 9790 wherein the medical device further comprises an echogenic material, and wherein the echogenic material is in the form of a coating.

9984. The method of item 9790 wherein the medical device is sterile.

9985. The method of item 9790 wherein the anti-scarring agent is released into tissue in the vicinity of the medical device after deployment of the medical device.

9986. The method of item 9790 wherein the anti-scarring agent is released into tissue in the vicinity of the medical device after deployment of the medical device, and wherein the tissue is connective tissue.

9987. The method of item 9790 wherein the anti-scarring agent is released into tissue in the vicinity of the medical device after deployment of the medical device, and wherein the tissue is muscle tissue.

9988. The method of item 9790 wherein the anti-scarring agent is released into tissue in the vicinity of the medical device after deployment of the medical device, and wherein the tissue is nerve tissue.

9989. The method of item 9790 wherein the anti-scarring agent is released into tissue in the vicinity of the medical device after deployment of the medical device, and wherein the tissue is epithelium tissue.

9990. The method of item 9790 wherein the anti-scarring agent is released in effective concentrations from the medical device over a period ranging from the time of deployment of the medical device to about 1 year.

9991. The method of item 9790 wherein the anti-scarring agent is released in effective concentrations from the medical device over a period ranging from about 1 month to 6 months.

9992. The method of item 9790 wherein the anti-scarring agent is released in effective concentrations from the medical device over a period ranging from about 1-90 days.

9993. The method of item 9790 wherein the anti-scarring agent is released in effective concentrations from the medical device at a constant rate.

9994. The method of item 9790 wherein the anti-scarring agent is released in effective concentrations from the medical device at an increasing rate.

9995. The method of item 9790 wherein the anti-scarring agent is released in effective concentrations from the medical device at a decreasing rate.

9996. The method of item 9790 wherein the anti-scarring agent is released in effective concentrations from the composition comprising the anti-scarring agent by diffusion over a period ranging from the time of deployment of the medical device to about 90 days.

9997. The method of item 9790 wherein the anti-scarring agent is released in effective concentrations from the composition comprising the anti-scarring agent by erosion of the composition over a period ranging from the time of deployment of the medical device to about 90 days.

9998. The method of item 9790 wherein the medical device comprises about 0.01 μg to about 10 μg of the anti-scarring agent.

9999. The method of item 9790 wherein the medical device comprises about 10 μg to about 10 mg of the anti-scarring agent.

10000. The method of item 9790 wherein the medical device comprises about 10 mg to about 250 mg of the anti-scarring agent.

10001. The method of item 9790 wherein the medical device comprises about 250 mg to about 1000 mg of the anti-scarring agent.

10002. The method of item 9790 wherein the medical device comprises about 1000 mg to about 2500 mg of the anti-scarring agent.

10003. The method of item 9790 wherein a surface of the medical device comprises less than 0.01 μg of the anti-scarring agent per mm² of medical device surface to which the anti-scarring agent is applied.

10004. The method of item 9790 wherein a surface of the medical device comprises about 0.01 μg to about 1 μg of the anti-scarring agent per mm² of medical device surface to which the anti-scarring agent is applied.

10005. The method of item 9790 wherein a surface of the medical device comprises about 1 μg to about 10 μg of the anti-scarring agent per mm² of medical device surface to which the anti-scarring agent is applied.

10006. The method of item 9790 wherein a surface of the medical device comprises about 10 μg to about 250 μg of the anti-scarring agent per mm² of medical device surface to which the anti-scarring agent is applied.

10007. The method of item 9790 wherein a surface of the medical device comprises about 250 μg to about 1000 μg of the anti-scarring agent of anti-scarring agent per mm² of medical device surface to which the anti-scarring agent is applied.

10008. The method of item 9790 wherein a surface of the medical device comprises about 10001g to about 2500 μg of the anti-scarring agent per mm² of medical device surface to which the anti-scarring agent is applied.

10009. The method of item 9790 wherein the combining is performed by direct affixing the agent or the composition to the electrical device.

10010. The method of item 9790 wherein the combining is performed by spraying the agent or the component onto the electrical device.

10011. The method of item 9790 wherein the combining is performed by electrospraying the agent or the composition onto the electrical device.

10012. The method of item 9790 wherein the combining is performed by dipping the electrical device into a solution comprising the agent or the composition.

10013. The method of item 9790 wherein the combining is performed by covalently attaching the agent or the composition to the electrical device.

10014. The method of item 9790 wherein the combining is performed by non-covalently attaching the agent or the composition to the electrical device.

10015. The method of item 9790 wherein the combining is performed by coating the electrical device with a substance that contains the agent or the composition.

10016. The method of item 9790 wherein the combining is performed by coating the electrical device with a substance that absorbs the agent.

10017. The method of item 9790 wherein the combining is performed by interweaving the electrical device with a thread composed of, or coated with, the agent or the composition.

10018. The method of item 9790 wherein the combining is performed by completely covering the electrical device with a sleeve that contains the agent or the composition.

10019. The method of item 9790 wherein the combining is performed by covering a portion of the electrical device with a sleeve that contains the agent or the composition.

10020. The method of item 9790 wherein the combining is performed by completely covering the electrical device with a cover that contains the agent or the composition.

10021. The method of item 9790 wherein the combining is performed by covering a portion of the electrical device with a cover that contains the agent or the composition.

10022. The method of item 9790 wherein the combining is performed by completely covering the electrical device with an electrospun fabric that contains the agent or the composition.

10023. The method of item 9790 wherein the combining is performed by covering a portion of the electrical device with an electrospun fabric that contains the agent or the composition.

10024. The method of item 9790 wherein the combining is performed by completely covering the electrical device with a mesh that contains the agent or the composition.

10025. The method of item 9790 wherein the combining is performed by covering a portion of the electrical device with a mesh that contains the agent or the composition.

10026. The method of item 9790 wherein the combining is performed by constructing a portion of the electrical device with the agent or the composition.

10027. The method of item 9790 wherein the combining is performed by impregnating the electrical device with the agent or the composition.

10028. The method of item 9790 wherein the combining is performed by constructing a portion of the electrical device from a degradable polymer that releases the agent.

10029. The method of item 9790 wherein the combining is performed by dipping the electrical device into a solution that comprise the agent and an inert solvent for the electrical device.

10030. The method of item 9790 wherein the combining is performed by dipping the electrical device into a solution that comprises the agent and a solvent that will swell the electrical device.

10031. The method of item 9790 wherein the combining is performed by dipping the electrical device into a solution that comprises the agent and a solvent that will dissolve the electrical device.

10032. The method of item 9790 wherein the combining is performed by dipping the electrical device into a solution that comprises the agent, a polymer and an inert solvent for the electrical device.

10033. The method of item 9790 wherein the combining is performed by dipping the electrical device into a solution that comprises the agent, a polymer and a solvent that will swell the electrical device.

10034. The method of item 9790 wherein the combining is performed by dipping the electrical device into a solution that comprises the agent, a polymer and a solvent that will dissolve the electrical device.

10035. The method of item 9790 wherein the combining is performed by spraying the electrical device into a solution that comprises the agent and an inert solvent for the electrical device.

10036. The method of item 9790 wherein the combining is performed by spraying the electrical device into a solution that comprises the agent and a solvent that will swell the electrical device.

10037. The method of item 9790 wherein the combining is performed by spraying the electrical device into a solution that comprises the agent and a solvent that will dissolve the electrical device.

10038. The method of item 9790 wherein the combining is performed by spraying the electrical device into a solution that comprises the agent, a polymer and an inert solvent for the electrical device.

10039. The method of item 9790 wherein the combining is performed by spraying the electrical device into a solution that comprises the agent, a polymer and a solvent that will swell the electrical device.

10040. The method of item 9790 wherein the combining is performed by spraying the electrical device into a solution that comprises the agent, a polymer and a solvent that will dissolve the electrical device.

10041. The method for making a medical device of any one of items 9790-10040 wherein the cardiac pacemaker is an adaptive rate pacemaker.

10042. The method for making a medical device of any one of items 9790-10040 wherein the cardiac pacemaker is a rate responsive pacemaker.

10043. A method for making a medical device comprising: combining an implantable cardioverter defibrillator (ICD) system (i.e., an electrical device) and an anti-scarring agent or a composition comprising an anti-scarring agent, wherein the agent inhibits scarring between the device and a host into which the device is implanted.

10044. The method of item 10043 wherein the agent is an adensosine A2A receptor antagonist.

10045. The method of item 10043 wherein the agent is an AKT inhibitor.

10046. The method of item 10043 wherein the agent is an alpha 2 integrin antagonist, wherein the alpha 2 integrin antagonist is Pharmaprojects No. 5754 (Merck KgaA).

10047. The method of item 10043 wherein the agent is an alpha 4 integrin antagonist.

10048. The method of item 10043 wherein the agent is an alpha 7 nicotinic receptor agonist.

10049. The method of item 10043 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

10050. The method of item 10043 wherein the agent is an apoptosis antagonist.

10051. The method of item 10043 wherein the agent is an apoptosis activator.

10052. The method of item 10043 wherein the agent is a beta 1 integrin antagonist.

10053. The method of item 10043 wherein the agent is a beta tubulin inhibitor.

10054. The method of item 10043 wherein the agent is a blocker of enzyme production in Hepatitis C.

10055. The method of item 10043 wherein the agent is a Bruton's tyrosine kinase inhibitor.

10056. The method of item 10043 wherein the agent is a calcineurin inhibitor.

10057. The method of item 10043 wherein the agent is a caspase 3 inhibitor.

10058. The method of item 10043 wherein the agent is a CC chemokine receptor antagonist.

10059. The method of item 10043 wherein the agent is a cell cycle inhibitor selected from the group consisting of SNS-595 (Sunesis), synthadotin, KRX-0403, and an analogue or derivative thereof.

10060. The method of item 10043 wherein the agent is a cathepsin B inhibitor.

10061. The method of item 10043 wherein the agent is a cathepsin K inhibitor, wherein the cathepsin K inhibitor is 462795 (GlaxoSmithKline), INPL-022-D6 (Amura Therapeutics), or an analogue or derivative thereof.

10062. The method of item 10043 wherein the agent is a cathepsin L inhibitor.

10063. The method of item 10043 wherein the agent is a CD40 antagonist.

10064. The method of item 10043 wherein the agent is a chemokine receptor agonist.

10065. The method of item 10043 wherein the agent is a chymase inhibitor.

10066. The method of item 10043 wherein the agent is a collagenase antagonist.

10067. The method of item 10043 wherein the agent is a CXCR antagonist.

10068. The method of item 10043 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

10069. The method of item 10043 wherein the agent is a cyclooxygenase 1 inhibitor.

10070. The method of item 10043 wherein the agent is a DHFR inhibitor.

10071. The method of item 10043 wherein the agent is a dual integrin inhibitor.

10072. The method of item 10043 wherein the agent is an elastase inhibitor.

10073. The method of item 10043 wherein the agent is an elongation factor-1 alpha inhibitor.

10074. The method of item 10043 wherein the agent is an endothelial growth factor antagonist.

10075. The method of item 10043 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

10076. The method of item 10043 wherein the agent is an endotoxin antagonist.

10077. The method of item 10043 wherein the agent is an epothilone and tubulin binder.

10078. The method of item 10043 wherein the agent is an estrogen receptor antagonist.

10079. The method of item 10043 wherein the agent is an FGF inhibitor.

10080. The method of item 10043 wherein the agent is a farnexyl transferase inhibitor.

10081. The method of item 10043 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

10082. The method of item 10043 wherein the agent is an FLT-3 kinase inhibitor.

10083. The method of item 10043 wherein the agent is an FGF receptor kinase inhibitor.

10084. The method of item 10043 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

10085. The method of item 10043 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

10086. The method of item 10043 wherein the agent is a histone deacetylase inhibitor.

10087. The method of item 10043 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

10088. The method of item 10043 wherein the agent is an ICAM inhibitor.

10089. The method of item 10043 wherein the agent is an IL, ICE and IRAK antagonist, wherein the antagonist is a CJ-14877, CP-424174 (Pfizer), NF-61 (Negma-Lerads), and an analogue or derivative thereof.

10090. The method of item 10043 wherein the agent is an IL-2 inhibitor.

10091. The method of item 10043 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

10092. The method of item 10043 wherein the agent is an IMPDH (inosine monophosphate).

10093. The method of item 10043 wherein the agent is an integrin antagonist.

10094. The method of item 10043 wherein the agent is an interleukin antagonist.

10095. The method of item 10043 wherein the agent is an inhibitor of type III receptor tyrosine kinase.

10096. The method of item 10043 wherein the agent is an irreversible inhibitor of enzyme methionine aminopeptidase type 2.

10097. The method of item 10043 wherein the agent is an isozyme selective delta protein kinase C inhibitor.

10098. The method of item 10043 wherein the agent a JAK3 enzyme inhibitor.

10099. The method of item 10043 wherein the agent is a JNK inhibitor.

10100. The method of item 10043 wherein the agent is a kinase inhibitor.

10101. The method of item 10043 wherein the agent is kinesin antagonist.

10102. The method of item 10043 wherein the agent is a kinesin antagonist.

10103. The method of item 10043 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

10104. The method of item 10043 wherein the agent is an MAP kinase inhibitor.

10105. The method of item 10043 wherein the agent is a matrix metalloproteinase inhibitor.

10106. The method of item 10043 wherein the agent is an MCP-CCR2 inhibitor.

10107. The method of item 10043 wherein the agent is an mTOR inhibitor.

10108. The method of item 10043 wherein the agent is an mTOR kinase inhibitor.

10109. The method of item 10043 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

10110. The method of item 10043 wherein the agent is an MIF inhibitor.

10111. The method of item 10043 wherein the agent is an MMP inhibitor.

10112. The method of item 10043 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

10113. The method of item 10043 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

10114. The method of item 10043 wherein the agent is a nitric oxide agonist.

10115. The method of item 10043 wherein the agent is an ornithine decarboxylase inhibitor.

10116. The method of item 10043 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

10117. The method of item 10043 wherein the agent is a palmitoyl-protein thioesterase inhibitor.

10118. The method of item 10043 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

10119. The method of item 10043 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

10120. The method of item 10043 wherein the agent is a phosphatase inhibitor.

10121. The method of item 10043 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

10122. The method of item 10043 wherein the agent is a PKC inhibitor.

10123. The method of item 10043 wherein the agent is a platelet activating factor antagonist.

10124. The method of item 10043 wherein the agent is a platelet-derived growth factor receptor kinase inhibitor.

10125. The method of item 10043 wherein the agent is a prolyl hydroxylase inhibitor.

10126. The method of item 10043 wherein the agent is a polymorphonuclear neutrophil inhibitor.

10127. The method of item 10043 wherein the agent is a protein kinase B inhibitor.

10128. The method of item 10043 wherein the agent is a protein kinase C stimulant.

10129. The method of item 10043 wherein the agent is a purine nucleoside analogue.

10130. The method of item 10043 wherein the agent is a purinoreceptor P2X antagonist.

10131. The method of item 10043 wherein the agent is a Raf kinase inhibitor.

10132. The method of item 10043 wherein the agent is a reversible inhibitor of ErbB1 and ErbB2.

10133. The method of item 10043 wherein the agent is a ribonucleoside triphosphate reductase inhibitor.

10134. The method of item 10043 wherein the agent is an SDF-1 antagonist.

10135. The method of item 10043 wherein the agent is a sheddase inhibitor.

10136. The method of item 10043 wherein the agent is an SRC inhibitor.

10137. The method of item 10043 wherein the agent is a stromelysin inhibitor.

10138. The method of item 10043 wherein the agent is an Syk kinase inhibitor.

10139. The method of item 10043 wherein the agent is a telomerase inhibitor.

10140. The method of item 10043 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

10141. The method of item 10043 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

10142. The method of item 10043 wherein the agent is a Toll receptor inhibitor.

10143. The method of item 10043 wherein the agent is a tubulin antagonist

10144. The method of item 10043 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

10145. The method of item 10043 wherein the agent is a VEGF inhibitor.

10146. The method of item 10043 wherein the agent is a vitamin D receptor agonist.

10147. The method of item 10043 wherein the agent is ZD-6474 (an angiogenesis inhibitor).

10148. The method of item 10043 wherein the agent is AP-23573 (an mTOR inhibitor).

10149. The method of item 10043 wherein the agent is synthadotin (a tubulin antagonist).

10150. The method of item 10043 wherein the agent is S-0885 (a collagenase inhibitor).

10151. The method of item 10043 wherein the agent is aplidine (an elongation factor-1 alpha inhibitor).

10152. The method of item 10043 wherein the agent is ixabepilone (an epithilone).

10153. The method of item 10043 wherein the agent is IDN-5390 (an angiogenesis inhibitor).

10154. The method of item 10043 wherein the agent is SB-2723005 (an angiogenesis inhibitor).

10155. The method of item 10043 wherein the agent is ABT-518 (an angiogenesis inhibitor).

10156. The method of item 10043 wherein the agent is combretastatin (an angiogenesis inhibitor).

10157. The method of item 10043 wherein the agent is anecortave acetate (an angiogenesis inhibitor).

10158. The method of item 10043 wherein the agent is SB-715992 (a kinesin antagonist).

10159. The method of item 10043 wherein the agent is temsirolimus (an mTOR inhibitor).

10160. The method of item 10043 wherein the agent is adalimumab (a TNFα antagonist).

10161. The method of item 10043, wherein the composition comprises a polymer.

10162. The method of item 10043, wherein the composition comprises a polymeric carrier.

10163. The method of item 10043 wherein the anti-scarring agent inhibits adhesion between the medical device and a host into which the medical device is implanted.

10164. The method of item 10043 wherein the medical device delivers the anti-scarring agent locally to tissue proximate to the medical device.

10165. The method of item 10043 wherein the medical device has a coating that comprises the anti-scarring agent

10166. The method of item 10043, wherein the medical device has a coating that comprises the agent and is disposed on a surface of the electrical device.

10167. The method of item 10043, wherein the medical device has a coating that comprises the agent and directly contacts the electrical device.

10168. The method of item 10043, wherein the medical device has a coating that comprises the agent and indirectly contacts the electrical device.

10169. The method of item 10043, wherein the medical device has a coating that comprises the agent and partially covers the electrical device.

10170. The method of item 10043, wherein the medical device has a coating that comprises the agent and completely covers the electrical device.

10171. The method of item 10043, wherein the medical device has a uniform coating.

10172. The method of item 10043, wherein the medical device has a non-uniform coating.

10173. The method of item 10043, wherein the medical device has a discontinuous coating.

10174. The method of item 10043, wherein the medical device has a patterned coating.

10175. The method of item 10043, wherein the medical device has a coating with a thickness of 100 μm or less.

10176. The method of item 10043, wherein the medical device has a coating with a thickness of 10 μm or less.

10177. The method of item 10043, wherein the medical device has a coating, and the coating adheres to the surface of the electrical device upon deployment of the electrical device.

10178. The method of item 10043, wherein the medical device has a coating, and wherein the coating is stable at room temperature for a period of 1 year.

10179. The method of item 10043, wherein the medical device has a coating, and wherein the anti-scarring agent is present in the coating in an amount ranging between about 0.0001% to about 1% by weight.

10180. The method of item 10043, wherein the medical device has a coating, and wherein the anti-scarring agent is present in the coating in an amount ranging between about 1% to about 10% by weight.

10181. The method of item 10043, wherein the medical device has a coating, and wherein the anti-scarring agent is present in the coating in an amount ranging between about 10% to about 25% by weight.

10182. The method of item 10043, wherein the medical device has a coating, and wherein the anti-scarring agent is present in the coating in an amount ranging between about 25% to about 70% by weight.

10183. The method of item 10043, wherein the medical device has a coating, and wherein the coating further comprises a polymer.

10184. The method of item 10043, wherein the medical device has a first coating having a first composition and a second coating having a second composition.

10185. The method of item 10043, wherein the medical device has a first coating having a first composition and a second coating having a second composition, wherein the first composition and the second composition are different.

10186. The method of item 10043, wherein the composition comprises a polymer.

10187. The method of item 10043, wherein the composition comprises a polymeric carrier.

10188. The method of item 10043, wherein the composition comprises a polymeric carrier, and wherein the polymeric carrier comprises a copolymer.

10189. The method of item 10043, wherein the composition comprises a polymeric carrier, and wherein the polymeric carrier comprises a block copolymer.

10190. The method of item 10043, wherein the composition comprises a polymeric carrier, and wherein the polymeric carrier comprises a random copolymer.

10191. The method of item 10043, wherein the composition comprises a polymeric carrier, and wherein the polymeric carrier comprises a biodegradable polymer.

10192. The method of item 10043, wherein the composition comprises a polymeric carrier, and wherein the polymeric carrier comprises a non-biodegradable polymer.

10193. The method of item 10043, wherein the composition comprises a polymeric carrier, and wherein the polymeric carrier comprises a hydrophilic polymer.

10194. The method of item 10043, wherein the composition comprises a polymeric carrier, and wherein the polymeric carrier comprises a hydrophobic polymer.

10195. The method of item 10043, wherein the composition comprises a polymeric carrier, and wherein the polymeric carrier comprises a polymer having hydrophilic domains.

10196. The method of item 10043, wherein the composition comprises a polymeric carrier, and wherein the polymeric carrier comprises a polymer having hydrophobic domains.

10197. The method of item 10043, wherein the composition comprises a polymeric carrier, and wherein the polymeric carrier comprises a non-conductive polymer.

10198. The method of item 10043, wherein the composition comprises a polymeric carrier, and wherein the polymeric carrier comprises an elastomer.

10199. The method of item 10043, wherein the composition comprises a polymeric carrier, and wherein the polymeric carrier comprises a hydrogel.

10200. The method of item 10043, wherein the composition comprises a polymeric carrier, and wherein the polymeric carrier comprises a silicone polymer.

10201. The method of item 10043, wherein the composition comprises a polymeric carrier, and wherein the polymeric carrier comprises a hydrocarbon polymer.

10202. The method of item 10043, wherein the composition comprises a polymeric carrier, and wherein the polymeric carrier comprises a styrene-derived polymer.

10203. The method of item 10043, wherein the composition comprises a polymeric carrier, and wherein the polymeric carrier comprises a butadiene polymer.

10204. The method of item 10043, wherein the composition comprises a polymeric carrier, and wherein the polymeric carrier comprises a macromer.

10205. The method of item 10043, wherein the composition comprises a polymeric carrier, and wherein the polymeric carrier comprises a poly(ethylene glycol) polymer.

10206. The method of item 10043 wherein the composition comprises a polymeric carrier, and wherein the polymeric carrier comprises an amorphous polymer.

10207. The method of item 10043, wherein the medical device comprises a lubricious coating.

10208. The method of item 10043 wherein the anti-scarring agent is located within pores or holes of the medical device.

10209. The method of item 10043 wherein the anti-scarring agent is located within a channel, lumen, or divet of the medical device.

10210. The method of item 10043, wherein the medical device further comprises a second pharmaceutically active agent.

10211. The method of item 10043 wherein the medical device further comprises an anti-inflammatory agent.

10212. The method of item 10043 wherein the medical device further comprises an agent that inhibits infection.

10213. The method of item 10043 wherein the medical device further comprises an agent that inhibits infection, and wherein the agent is an anthracycline.

10214. The method of item 10043 wherein the medical device further comprises an agent that inhibits infection, and wherein the agent is doxorubicin.

10215. The method of item 10043 wherein the medical device further comprises an agent that inhibits infection, and wherein the agent is mitoxantrone.

10216. The method of item 10043 wherein the medical device further comprises an agent that inhibits infection, and wherein the agent is a fluoropyrimidine.

10217. The method of item 10043 wherein the medical device further comprises an agent that inhibits infection, and wherein the agent is 5-fluorouracil (5-FU).

10218. The method of item 10043 wherein the medical device further comprises an agent that inhibits infection, and wherein the agent is a folic acid antagonist.

10219. The method of item 10043 wherein the medical device further comprises an agent that inhibits infection, and wherein the agent is methotrexate.

10220. The method of item 10043 wherein the medical device further comprises an agent that inhibits infection, and wherein the agent is a podophylotoxin.

10221. The method of item 10043 wherein the medical device further comprises an agent that inhibits infection, and wherein the agent is etoposide.

10222. The method of item 10043 wherein the medical device further comprises an agent that inhibits infection, and wherein the agent is a camptothecin.

10223. The method of item 10043 wherein the medical device further comprises an agent that inhibits infection, and wherein the agent is a hydroxyurea.

10224. The method of item 10043 wherein the medical device further comprises an agent that inhibits infection, and wherein the agent is a platinum complex.

10225. The method of item 10043 wherein the medical device further comprises an agent that inhibits infection, and wherein the agent is cisplatin.

10226. The method of item 10043 wherein the medical device further comprises an anti-thrombotic agent.

10227. The method of item 10043 wherein the medical device further comprises a visualization agent.

10228. The method of item 10043 wherein the medical device further comprises a visualization agent, wherein the visualization agent is a radiopaque material, and wherein the radiopaque material further comprises a metal, a halogenated compound, or a barium containing compound.

10229. The method of item 10043 wherein the medical device further comprises a visualization agent, wherein the visualization agent is a radiopaque material, and wherein the radiopaque material further comprises barium, tantalum, or technetium.

10230. The method of item 10043 wherein the medical device further comprises a visualization agent, and wherein the visualization agent is a MRI responsive material.

10231. The method of item 10043 wherein the medical device further comprises a visualization agent, and wherein the visualization agent further comprises a gadolinium chelate.

10232. The method of item 10043 wherein the medical device further comprises a visualization agent, and wherein the visualization agent further comprises iron, magnesium, manganese, copper, or chromium.

10233. The method of item 10043 wherein the medical device further comprises a visualization agent, and wherein the visualization agent further comprises an iron oxide compound.

10234. The method of item 10043 wherein the medical device further comprises a visualization agent, and wherein the visualization agent further comprises a dye, pigment, or colorant.

10235. The method of item 10043 wherein the medical device further comprises an echogenic material.

10236. The method of item 10043 wherein the medical device further comprises an echogenic material, and wherein the echogenic material is in the form of a coating.

10237. The method of item 10043 wherein the medical device is sterile.

10238. The method of item 10043 wherein the anti-scarring agent is released into tissue in the vicinity of the medical device after deployment of the medical device.

10239. The method of item 10043 wherein the anti-scarring agent is released into tissue in the vicinity of the medical device after deployment of the medical device, and wherein the tissue is connective tissue.

10240. The method of item 10043 wherein the anti-scarring agent is released into tissue in the vicinity of the medical device after deployment of the medical device, and wherein the tissue is muscle tissue.

10241. The method of item 10043 wherein the anti-scarring agent is released into tissue in the vicinity of the medical device after deployment of the medical device, and wherein the tissue is nerve tissue.

10242. The method of item 10043 wherein the anti-scarring agent is released into tissue in the vicinity of the medical device after deployment of the medical device, and wherein the tissue is epithelium tissue.

10243. The method of item 10043 wherein the anti-scarring agent is released in effective concentrations from the medical device over a period ranging from the time of deployment of the medical device to about 1 year.

10244. The method of item 10043 wherein the anti-scarring agent is released in effective concentrations from the medical device over a period ranging from about 1 month to 6 months.

10245. The method of item 10043 wherein the anti-scarring agent is released in effective concentrations from the medical device over a period ranging from about 1-90 days.

10246. The method of item 10043 wherein the anti-scarring agent is released in effective concentrations from the medical device at a constant rate.

10247. The method of item 10043 wherein the anti-scarring agent is released in effective concentrations from the medical device at an increasing rate.

10248. The method of item 10043 wherein the anti-scarring agent is released in effective concentrations from the medical device at a decreasing rate.

10249. The method of item 10043 wherein the anti-scarring agent is released in effective concentrations from the composition comprising the anti-scarring agent by diffusion over a period ranging from the time of deployment of the medical device to about 90 days.

10250. The method of item 10043 wherein the anti-scarring agent is released in effective concentrations from the composition comprising the anti-scarring agent by erosion of the composition over a period ranging from the time of deployment of the medical device to about 90 days.

10251. The method of item 10043 wherein the medical device comprises about 0.01 μg to about 10 μg of the anti-scarring agent.

10252. The method of item 10043 wherein the medical device comprises about 10 μg to about 10 mg of the anti-scarring agent.

10253. The method of item 10043 wherein the medical device comprises about 10 mg to about 250 mg of the anti-scarring agent.

10254. The method of item 10043 wherein the medical device comprises about 250 mg to about 1000 mg of the anti-scarring agent.

10255. The method of item 10043 wherein the medical device comprises about 1000 mg to about 2500 mg of the anti-scarring agent.

10256. The method of item 10043 wherein a surface of the medical device comprises less than 0.01 μg of the anti-scarring agent per mm² of medical device surface to which the anti-scarring agent is applied.

10257. The method of item 10043 wherein a surface of the medical device comprises about 0.01 μg to about 1 μg of the anti-scarring agent per mm² of medical device surface to which the anti-scarring agent is applied.

10258. The method of item 10043 wherein a surface of the medical device comprises about 1 μg to about 10 μg of the anti-scarring agent per mm² of medical device surface to which the anti-scarring agent is applied.

10259. The method of item 10043 wherein a surface of the medical device comprises about 10 μg to about 250 μg of the anti-scarring agent per mm² of medical device surface to which the anti-scarring agent is applied.

10260. The method of item 10043 wherein a surface of the medical device comprises about 250 μg to about 1000 μg of the anti-scarring agent of anti-scarring agent per mm² of medical device surface to which the anti-scarring agent is applied.

10261. The method of item 10043 wherein a surface of the medical device comprises about 1000 μg to about 2500 μg of the anti-scarring agent per mm² of medical device surface to which the anti-scarring agent is applied.

10262. The method of item 10043 wherein the combining is performed by direct affixing the agent or the composition to the electrical device.

10263. The method of item 10043 wherein the combining is performed by spraying the agent or the component onto the electrical device.

10264. The method of item 10043 wherein the combining is performed by electrospraying the agent or the composition onto the electrical device.

10265. The method of item 10043 wherein the combining is performed by dipping the electrical device into a solution comprising the agent or the composition.

10266. The method of item 10043 wherein the combining is performed by covalently attaching the agent or the composition to the electrical device.

10267. The method of item 10043 wherein the combining is performed by non-covalently attaching the agent or the composition to the electrical device.

10268. The method of item 10043 wherein the combining is performed by coating the electrical device with a substance that contains the agent or the composition.

10269. The method of item 10043 wherein the combining is performed by coating the electrical device with a substance that absorbs the agent.

10270. The method of item 10043 wherein the combining is performed by interweaving the electrical device with a thread composed of, or coated with, the agent or the composition.

10271. The method of item 10043 wherein the combining is performed by completely covering the electrical device with a sleeve that contains the agent or the composition.

10272. The method of item 10043 wherein the combining is performed by covering a portion of the electrical device with a sleeve that contains the agent or the composition.

10273. The method of item 10043 wherein the combining is performed by completely covering the electrical device with a cover that contains the agent or the composition.

10274. The method of item 10043 wherein the combining is performed by covering a portion of the electrical device with a cover that contains the agent or the composition.

10275. The method of item 10043 wherein the combining is performed by completely covering the electrical device with an electrospun fabric that contains the agent or the composition.

10276. The method of item 10043 wherein the combining is performed by covering a portion of the electrical device with an electrospun fabric that contains the agent or the composition.

10277. The method of item 10043 wherein the combining is performed by completely covering the electrical device with a mesh that contains the agent or the composition.

10278. The method of item 10043 wherein the combining is performed by covering a portion of the electrical device with a mesh that contains the agent or the composition.

10279. The method of item 10043 wherein the combining is performed by constructing a portion of the electrical device with the agent or the composition.

10280. The method of item 10043 wherein the combining is performed by impregnating the electrical device with the agent or the composition.

10281. The method of item 10043 wherein the combining is performed by constructing a portion of the electrical device from a degradable polymer that releases the agent.

10282. The method of item 10043 wherein the combining is performed by dipping the electrical device into a solution that comprise the agent and an inert solvent for the electrical device.

10283. The method of item 10043 wherein the combining is performed by dipping the electrical device into a solution that comprises the agent and a solvent that will swell the electrical device.

10284. The method of item 10043 wherein the combining is performed by dipping the electrical device into a solution that comprises the agent and a solvent that will dissolve the electrical device.

10285. The method of item 10043 wherein the combining is performed by dipping the electrical device into a solution that comprises the agent, a polymer and an inert solvent for the electrical device.

10286. The method of item 10043 wherein the combining is performed by dipping the electrical device into a solution that comprises the agent, a polymer and a solvent that will swell the electrical device.

10287. The method of item 10043 wherein the combining is performed by dipping the electrical device into a solution that comprises the agent, a polymer and a solvent that will dissolve the electrical device.

10288. The method of item 10043 wherein the combining is performed by spraying the electrical device into a solution that comprises the agent and an inert solvent for the electrical device.

10289. The method of item 10043 wherein the combining is performed by spraying the electrical device into a solution that comprises the agent and a solvent that will swell the electrical device.

10290. The method of item 10043 wherein the combining is performed by spraying the electrical device into a solution that comprises the agent and a solvent that will dissolve the electrical device.

10291. The method of item 10043 wherein the combining is performed by spraying the electrical device into a solution that comprises the agent, a polymer and an inert solvent for the electrical device.

10292. The method of item 10043 wherein the combining is performed by spraying the electrical device into a solution that comprises the agent, a polymer and a solvent that will swell the electrical device.

10293. The method of item 10043 wherein the combining is performed by spraying the electrical device into a solution that comprises the agent, a polymer and a solvent that will dissolve the electrical device.

10294. The method for making a medical device of any one of items 10043-10293 wherein the implantable cardioverter defibrillator is adapted for treating tachyarraythmias.

10295. The method for making a medical device of any one of items 10043-10293 wherein the implantable cardioverter defibrillator is adapted for ventricular tachycardia.

10296. The method for making a medical device of any one of items 10043-10293 wherein the implantable cardioverter defibrillator is adapted for treating ventricular fibrillation.

10297. The method for making a medical device of any one of items 10043-10293 wherein the implantable cardioverter defibrillator is adapted for treating atrial tachycardia.

10298. The method for making a medical device of any one of items 10043-10293 wherein the implantable cardioverter defibrillator is adapted for treating atrial fibrillation

10299. The method for making a medical device of any one of items 10043-10293 wherein the implantable cardioverter defibrillator is adapted for treating arrhythmias.

10300. A method for making a medical device comprising: combining a vagus nerve stimulator for treating arrhythemia (i.e., an electrical device) and an anti-scarring agent or a composition comprising an anti-scarring agent, wherein the agent inhibits scarring between the device and a host into which the device is implanted.

10301. The method of item 10300 wherein the agent is an adensosine A2A receptor antagonist.

10302. The method of item 10300 wherein the agent is an AKT inhibitor.

10303. The method of item 10300 wherein the agent is an alpha 2 integrin antagonist, wherein the alpha 2 integrin antagonist is Pharmaprojects No. 5754 (Merck KgaA).

10304. The method of item 10300 wherein the agent is an alpha 4 integrin antagonist.

10305. The method of item 10300 wherein the agent is an alpha 7 nicotinic receptor agonist.

10306. The method of item 10300 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

10307. The method of item 10300 wherein the agent is an apoptosis antagonist.

10308. The method of item 10300 wherein the agent is an apoptosis activator.

10309. The method of item 10300 wherein the agent is a beta 1 integrin antagonist.

10310. The method of item 10300 wherein the agent is a beta tubulin inhibitor.

10311. The method of item 10300 wherein the agent is a blocker of enzyme production in Hepatitis C.

10312. The method of item 10300 wherein the agent is a Bruton's tyrosine kinase inhibitor.

10313. The method of item 10300 wherein the agent is a calcineurin inhibitor.

10314. The method of item 10300 wherein the agent is a caspase 3 inhibitor.

10315. The method of item 10300 wherein the agent is a CC chemokine receptor antagonist.

10316. The method of item 10300 wherein the agent is a cell cycle inhibitor selected from the group consisting of SNS-595 (Sunesis), synthadotin, KRX-0403, and an analogue or derivative thereof.

10317. The method of item 10300 wherein the agent is a cathepsin B inhibitor.

10318. The method of item 10300 wherein the agent is a cathepsin K inhibitor, wherein the cathepsin K inhibitor is 462795 (GlaxoSmithKline), INPL-022-D6 (Amura Therapeutics), or an analogue or derivative thereof.

10319. The method of item 10300 wherein the agent is a cathepsin L inhibitor.

10320. The method of item 10300 wherein the agent is a CD40 antagonist.

10321. The method of item 10300 wherein the agent is a chemokine receptor agonist.

10322. The method of item 10300 wherein the agent is a chymase inhibitor.

10323. The method of item 10300 wherein the agent is a collagenase antagonist.

10324. The method of item 10300 wherein the agent is a CXCR antagonist.

10325. The method of item 10300 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

10326. The method of item 10300 wherein the agent is a cyclooxygenase 1 inhibitor.

10327. The method of item 10300 wherein the agent is a DHFR inhibitor.

10328. The method of item 10300 wherein the agent is a dual integrin inhibitor.

10329. The method of item 10300 wherein the agent is an elastase inhibitor.

10330. The method of item 10300 wherein the agent is an elongation factor-1 alpha inhibitor.

10331. The method of item 10300 wherein the agent is an endothelial growth factor antagonist.

10332. The method of item 10300 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

10333. The method of item 10300 wherein the agent is an endotoxin antagonist.

10334. The method of item 10300 wherein the agent is an epothilone and tubulin binder.

10335. The method of item 10300 wherein the agent is an estrogen receptor antagonist.

10336. The method of item 10300 wherein the agent is an FGF inhibitor.

10337. The method of item 10300 wherein the agent is a farnexyl transferase inhibitor.

10338. The method of item 10300 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

10339. The method of item 10300 wherein the agent is an FLT-3 kinase inhibitor.

10340. The method of item 10300 wherein the agent is an FGF receptor kinase inhibitor.

10341. The method of item 10300 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

10342. The method of item 10300 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

10343. The method of item 10300 wherein the agent is a histone deacetylase inhibitor.

10344. The method of item 10300 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

10345. The method of item 10300 wherein the agent is an ICAM inhibitor.

10346. The method of item 10300 wherein the agent is an IL, ICE and IRAK antagonist, wherein the antagonist is a CJ-14877, CP-424174 (Pfizer), NF-61 (Negma-Lerads), and an analogue or derivative thereof

10347. The method of item 10300 wherein the agent is an IL-2 inhibitor.

10348. The method of item 10300 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

10349. The method of item 10300 wherein the agent is an IMPDH (inosine monophosphate).

10350. The method of item 10300 wherein the agent is an integrin antagonist.

10351. The method of item 10300 wherein the agent is an interleukin antagonist.

10352. The method of item 10300 wherein the agent is an inhibitor of type III receptor tyrosine kinase.

10353. The method of item 10300 wherein the agent is an irreversible inhibitor of enzyme methionine aminopeptidase type 2.

10354. The method of item 10300 wherein the agent is an isozyme selective delta protein kinase C inhibitor.

10355. The method of item 10300 wherein the agent a JAK3 enzyme inhibitor.

10356. The method of item 10300 wherein the agent is a JNK inhibitor.

10357. The method of item 10300 wherein the agent is a kinase inhibitor.

10358. The method of item 10300 wherein the agent is kinesin antagonist.

10359. The method of item 10300 wherein the agent is a kinesin antagonist.

10360. The method of item 10300 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

10361. The method of item 10300 wherein the agent is an MAP kinase inhibitor.

10362. The method of item 10300 wherein the agent is a matrix metalloproteinase inhibitor.

10363. The method of item 10300 wherein the agent is an MCP-CCR2 inhibitor.

10364. The method of item 10300 wherein the agent is an mTOR inhibitor.

10365. The method of item 10300 wherein the agent is an mTOR kinase inhibitor.

10366. The method of item 10300 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

10367. The method of item 10300 wherein the agent is an MIF inhibitor.

10368. The method of item 10300 wherein the agent is an MMP inhibitor.

10369. The method of item 10300 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

10370. The method of item 10300 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

10371. The method of item 10300 wherein the agent is a nitric oxide agonist.

10372. The method of item 10300 wherein the agent is an ornithine decarboxylase inhibitor.

10373. The method of item 10300 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

10374. The method of item 10300 wherein the agent is a palmitoyl-protein thioesterase inhibitor.

10375. The method of item 10300 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

10376. The method of item 10300 wherein the agent is (−)-halofenate (Metabolex), AMG-131 (Amgen), antidiabetics from Japan Tobacco, AZD-4619, AZD-8450, AZD-8677 (AstraZeneca), DRF-1 0945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

10377. The method of item 10300 wherein the agent is a phosphatase inhibitor.

10378. The method of item 10300 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WlN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

10379. The method of item 10300 wherein the agent is a PKC inhibitor.

10380. The method of item 10300 wherein the agent is a platelet activating factor antagonist.

10381. The method of item 10300 wherein the agent is a platelet-derived growth factor receptor kinase inhibitor.

10382. The method of item 10300 wherein the agent is a prolyl hydroxylase inhibitor.

10383. The method of item 10300 wherein the agent is a polymorphonuclear neutrophil inhibitor.

10384. The method of item 10300 wherein the agent is a protein kinase B inhibitor.

10385. The method of item 10300 wherein the agent is a protein kinase C stimulant.

10386. The method of item 10300 wherein the agent is a purine nucleoside analogue.

10387. The method of item 10300 wherein the agent is a purinoreceptor P2X antagonist.

10388. The method of item 10300 wherein the agent is a Raf kinase inhibitor.

10389. The method of item 10300 wherein the agent is a reversible inhibitor of ErbB1 and ErbB2.

10390. The method of item 10300 wherein the agent is a ribonucleoside triphosphate reductase inhibitor.

10391. The method of item 10300 wherein the agent is an SDF-1 antagonist.

10392. The method of item 10300 wherein the agent is a sheddase inhibitor.

10393. The method of item 10300 wherein the agent is an SRC inhibitor.

10394. The method of item 10300 wherein the agent is a stromelysin inhibitor.

10395. The method of item 10300 wherein the agent is an Syk kinase inhibitor.

10396. The method of item 10300 wherein the agent is a telomerase inhibitor.

10397. The method of item 10300 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

10398. The method of item 10300 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

10399. The method of item 10300 wherein the agent is a Toll receptor inhibitor.

10400. The method of item 10300 wherein the agent is a tubulin antagonist.

10401. The method of item 10300 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-10 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-1 3 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

10402. The method of item 10300 wherein the agent is a VEGF inhibitor.

10403. The method of item 10300 wherein the agent is a vitamin D receptor agonist.

10404. The method of item 10300 wherein the agent is ZD-6474 (an angiogenesis inhibitor).

10405. The method of item 10300 wherein the agent is AP-23573 (an mTOR inhibitor).

10406. The method of item 10300 wherein the agent is synthadotin (a tubulin antagonist).

10407. The method of item 10300 wherein the agent is S-0885 (a collagenase inhibitor).

10408. The method of item 10300 wherein the agent is aplidine (an elongation factor-1alpha inhibitor).

10409. The method of item 10300 wherein the agent is ixabepilone (an epithilone).

10410. The method of item 10300 wherein the agent is IDN-5390 (an angiogenesis inhibitor).

10411. The method of item 10300 wherein the agent is SB-2723005 (an angiogenesis inhibitor).

10412. The method of item 10300 wherein the agent is ABT-518 (an angiogenesis inhibitor).

10413. The method of item 10300 wherein the agent is combretastatin (an angiogenesis inhibitor).

10414. The method of item 10300 wherein the agent is anecortave acetate (an angiogenesis inhibitor).

10415. The method of item 10300 wherein the agent is SB-715992 (a kinesin antagonist).

10416. The method of item 10300 wherein the agent is temsirolimus (an mTOR inhibitor).

10417. The method of item 10300 wherein the agent is adalimumab (a TNFα antagonist).

10418. The method of item 10300, wherein the composition comprises a polymer.

10419. The method of item 10300, wherein the composition comprises a polymeric carrier.

10420. The method of item 10300 wherein the anti-scarring agent inhibits adhesion between the medical device and a host into which the medical device is implanted.

10421. The method of item 10300 wherein the medical device delivers the anti-scarring agent locally to tissue proximate to the medical device.

10422. The method of item 10300 wherein the medical device has a coating that comprises the anti-scarring agent.

10423. The method of item 10300, wherein the medical device has a coating that comprises the agent and is disposed on a surface of the electrical device.

10424. The method of item 10300, wherein the medical device has a coating that comprises the agent and directly contacts the electrical device.

10425. The method of item 10300, wherein the medical device has a coating that comprises the agent and indirectly contacts the electrical device.

10426. The method of item 10300, wherein the medical device has a coating that comprises the agent and partially covers the electrical device.

10427. The method of item 10300, wherein the medical device has a coating that comprises the agent and completely covers the electrical device.

10428. The method of item 10300, wherein the medical device has a uniform coating

10429. The method of item 10300, wherein the medical device has a non-uniform coating.

10430. The method of item 10300, wherein the medical device has a discontinuous coating.

10431. The method of item 10300, wherein the medical device has a patterned coating.

10432. The method of item 10300, wherein the medical device has a coating with a thickness of 100 μm or less.

10433. The method of item 10300, wherein the medical device has a coating with a thickness of 10 μm or less.

10434. The method of item 10300, wherein the medical device has a coating, and the coating adheres to the surface of the electrical device upon deployment of the electrical device

10435. The method of item 10300, wherein the medical device has a coating, and wherein the coating is stable at room temperature for a period of 1 year.

10436. The method of item 10300, wherein the medical device has a coating, and wherein the anti-scarring agent is present in the coating in an amount ranging between about 0.0001% to about 1% by weight.

10437. The method of item 10300, wherein the medical device has a coating, and wherein the anti-scarring agent is present in the coating in an amount ranging between about 1% to about 10% by weight.

10438. The method of item 10300, wherein the medical device has a coating, and wherein the anti-scarring agent is present in the coating in an amount ranging between about 10% to about 25% by weight.

10439. The method of item 10300, wherein the medical device has a coating, and wherein the anti-scarring agent is present in the coating in an amount ranging between about 25% to about 70% by weight.

10440. The method of item 10300, wherein the medical device has a coating, and wherein the coating further comprises a polymer.

10441. The method of item 10300, wherein the medical device has a first coating having a first composition and a second coating having a second composition.

10442. The method of item 10300, wherein the medical device has a first coating having a first composition and a second coating having a second composition, wherein the first composition and the second composition are different.

10443. The method of item 10300, wherein the composition comprises a polymer.

10444. The method of item 10300, wherein the composition comprises a polymeric carrier.

10445. The method of item 10300, wherein the composition comprises a polymeric carrier, and wherein the polymeric carrier comprises a copolymer.

10446. The method of item 10300, wherein the composition comprises a polymeric carrier, and wherein the polymeric carrier comprises a block copolymer.

10447. The method of item 10300, wherein the composition comprises a polymeric carrier, and wherein the polymeric carrier comprises a random copolymer.

10448. The method of item 10300, wherein the composition comprises a polymeric carrier, and wherein the polymeric carrier comprises a biodegradable polymer.

10449. The method of item 10300, wherein the composition comprises a polymeric carrier, and wherein the polymeric carrier comprises a non-biodegradable polymer.

10450. The method of item 10300, wherein the composition comprises a polymeric carrier, and wherein the polymeric carrier comprises a hydrophilic polymer.

10451. The method of item 10300, wherein the composition comprises a polymeric carrier, and wherein the polymeric carrier comprises a hydrophobic polymer.

10452. The method of item 10300, wherein the composition comprises a polymeric carrier, and wherein the polymeric carrier comprises a polymer having hydrophilic domains.

10453. The method of item 10300, wherein the composition comprises a polymeric carrier, and wherein the polymeric carrier comprises a polymer having hydrophobic domains.

10454. The method of item 10300, wherein the composition comprises a polymeric carrier, and wherein the polymeric carrier comprises a non-conductive polymer.

10455. The method of item 10300, wherein the composition comprises a polymeric carrier, and wherein the polymeric carrier comprises an elastomer.

10456. The method of item 10300, wherein the composition comprises a polymeric carrier, and wherein the polymeric carrier comprises a hydrogel.

10457. The method of item 10300, wherein the composition comprises a polymeric carrier, and wherein the polymeric carrier comprises a silicone polymer

10458. The method of item 10300, wherein the composition comprises a polymeric carrier, and wherein the polymeric carrier comprises a hydrocarbon polymer.

10459. The method of item 10300, wherein the composition comprises a polymeric carrier, and wherein the polymeric carrier comprises a styrene-derived polymer.

10460. The method of item 10300, wherein the composition comprises a polymeric carrier, and wherein the polymeric carrier comprises a butadiene polymer.

10461. The method of item 10300, wherein the composition comprises a polymeric carrier, and wherein the polymeric carrier comprises a macromer.

10462. The method of item 10300, wherein the composition comprises a polymeric carrier, and wherein the polymeric carrier comprises a poly(ethylene glycol) polymer.

10463. The method of item 10300 wherein the composition comprises a polymeric carrier, and wherein the polymeric carrier comprises an amorphous polymer.

10464. The method of item 10300, wherein the medical device comprises a lubricious coating.

10465. The method of item 10300 wherein the anti-scarring agent is located within pores or holes of the medical device.

10466. The method of item 10300 wherein the anti-scarring agent is located within a channel, lumen, or divet of the medical device.

10467. The method of item 10300, wherein the medical device further comprises a second pharmaceutically active agent.

10468. The method of item 10300 wherein the medical device further comprises an anti-inflammatory agent.

10469. The method of item 10300 wherein the medical device further comprises an agent that inhibits infection.

10470. The method of item 10300 wherein the medical device further comprises an agent that inhibits infection, and wherein the agent is an anthracycline.

10471. The method of item 10300 wherein the medical device further comprises an agent that inhibits infection, and wherein the agent is doxorubicin.

10472. The method of item 10300 wherein the medical device further comprises an agent that inhibits infection, and wherein the agent is mitoxantrone.

10473. The method of item 10300 wherein the medical device further comprises an agent that inhibits infection, and wherein the agent is a fluoropyrimidine.

10474. The method of item 10300 wherein the medical device further comprises an agent that inhibits infection, and wherein the agent is 5-fluorouracil (5-FU).

10475. The method of item 10300 wherein the medical device further comprises an agent that inhibits infection, and wherein the agent is a folic acid antagonist.

10476. The method of item 10300 wherein the medical device further comprises an agent that inhibits infection, and wherein the agent is methotrexate.

10477. The method of item 10300 wherein the medical device further comprises an agent that inhibits infection, and wherein the agent is a podophylotoxin.

10478. The method of item 10300 wherein the medical device further comprises an agent that inhibits infection, and wherein the agent is etoposide.

10479. The method of item 10300 wherein the medical device further comprises an agent that inhibits infection, and wherein the agent is a camptothecin.

10480. The method of item 10300 wherein the medical device further comprises an agent that inhibits infection, and wherein the agent is a hydroxyurea.

10481. The method of item 10300 wherein the medical device further comprises an agent that inhibits infection, and wherein the agent is a platinum complex.

10482. The method of item 10300 wherein the medical device further comprises an agent that inhibits infection, and wherein the agent is cisplatin.

10483. The method of item 10300 wherein the medical device further comprises an anti-thrombotic agent.

10484. The method of item 10300 wherein the medical device further comprises a visualization agent.

10485. The method of item 10300 wherein the medical device further comprises a visualization agent, wherein the visualization agent is a radiopaque material, and wherein the radiopaque material further comprises a metal, a halogenated compound, or a barium containing compound.

10486. The method of item 10300 wherein the medical device further comprises a visualization agent, wherein the visualization agent is a radiopaque material, and wherein the radiopaque material further comprises barium, tantalum, or technetium.

10487. The method of item 10300 wherein the medical device further comprises a visualization agent, and wherein the visualization agent is a MRI responsive material.

10488. The method of item 10300 wherein the medical device further comprises a visualization agent, and wherein the visualization agent further comprises a gadolinium chelate.

10489. The method of item 10300 wherein the medical device further comprises a visualization agent, and wherein the visualization agent further comprises iron, magnesium, manganese, copper, or chromium.

10490. The method of item 10300 wherein the medical device further comprises a visualization agent, and wherein the visualization agent further comprises an iron oxide compound.

10491. The method of item 10300 wherein the medical device further comprises a visualization agent, and wherein the visualization agent further comprises a dye, pigment, or colorant.

10492. The method of item 10300 wherein the medical device further comprises an echogenic material.

10493. The method of item 10300 wherein the medical device further comprises an echogenic material, and wherein the echogenic material is in the form of a coating.

10494. The method of item 10300 wherein the medical device is sterile.

10495. The method of item 10300 wherein the anti-scarring agent is released into tissue in the vicinity of the medical device after deployment of the medical device.

10496. The method of item 10300 wherein the anti-scarring agent is released into tissue in the vicinity of the medical device after deployment of the medical device, and wherein the tissue is connective tissue.

10497. The method of item 10300 wherein the anti-scarring agent is released into tissue in the vicinity of the medical device after deployment of the medical device, and wherein the tissue is muscle tissue.

10498. The method of item 10300 wherein the anti-scarring agent is released into tissue in the vicinity of the medical device after deployment of the medical device, and wherein the tissue is nerve tissue.

10499. The method of item 10300 wherein the anti-scarring agent is released into tissue in the vicinity of the medical device after deployment of the medical device, and wherein the tissue is epithelium tissue.

10500. The method of item 10300 wherein the anti-scarring agent is released in effective concentrations from the medical device over a period ranging from the time of deployment of the medical device to about 1 year.

10501. The method of item 10300 wherein the anti-scarring agent is released in effective concentrations from the medical device over a period ranging from about 1 month to 6 months.

10502. The method of item 10300 wherein the anti-scarring agent is released in effective concentrations from the medical device over a period ranging from about 1-90 days.

10503. The method of item 10300 wherein the anti-scarring agent is released in effective concentrations from the medical device at a constant rate.

10504. The method of item 10300 wherein the anti-scarring agent is released in effective concentrations from the medical device at an increasing rate.

10505. The method of item 10300 wherein the anti-scarring agent is released in effective concentrations from the medical device at a decreasing rate.

10506. The method of item 10300 wherein the anti-scarring agent is released in effective concentrations from the composition comprising the anti-scarring agent by diffusion over a period ranging from the time of deployment of the medical device to about 90 days.

10507. The method of item 10300 wherein the anti-scarring agent is released in effective concentrations from the composition comprising the anti-scarring agent by erosion of the composition over a period ranging from the time of deployment of the medical device to about 90 days.

10508. The method of item 10300 wherein the medical device comprises about 0.01 μg to about 10 μg of the anti-scarring agent.

10509. The method of item 10300 wherein the medical device comprises about 10 μg to about 10 mg of the anti-scarring agent.

10510. The method of item 10300 wherein the medical device comprises about 10 mg to about 250 mg of the anti-scarring agent.

10511. The method of item 10300 wherein the medical device comprises about 250 mg to about 1000 mg of the anti-scarring agent.

10512. The method of item 10300 wherein the medical device comprises about 1000 mg to about 2500 mg of the anti-scarring agent.

10513. The method of item 10300 wherein a surface of the medical device comprises less than 0.01 μg of the anti-scarring agent per mm² of medical device surface to which the anti-scarring agent is applied.

10514. The method of item 10300 wherein a surface of the medical device comprises about 0.01 μg to about 1 μg of the anti-scarring agent per mm² of medical device surface to which the anti-scarring agent is applied.

10515. The method of item 10300 wherein a surface of the medical device comprises about 1 μg to about 10 μg of the anti-scarring agent per mm² of medical device surface to which the anti-scarring agent is applied.

10516. The method of item 10300 wherein a surface of the medical device comprises about 10 μg to about 250 μg of the anti-scarring agent per mm² of medical device surface to which the anti-scarring agent is applied.

10517. The method of item 10300 wherein a surface of the medical device comprises about 250 μg to about 1000 μg of the anti-scarring agent of anti-scarring agent per mm² of medical device surface to which the anti-scarring agent is applied.

10518. The method of item 10300 wherein a surface of the medical device comprises about 1000 μg to about 2500 μg of the anti-scarring agent per mm² of medical device surface to which the anti-scarring agent is applied.

10519. The method of item 10300 wherein the combining is performed by direct affixing the agent or the composition to the electrical device.

10520. The method of item 10300 wherein the combining is performed by spraying the agent or the component onto the electrical device.

10521. The method of item 10300 wherein the combining is performed by electrospraying the agent or the composition onto the electrical device.

10522. The method of item 10300 wherein the combining is performed by dipping the electrical device into a solution comprising the agent or the composition.

10523. The method of item 10300 wherein the combining is performed by covalently attaching the agent or the composition to the electrical device.

10524. The method of item 10300 wherein the combining is performed by non-covalently attaching the agent or the composition to the electrical device.

10525. The method of item 10300 wherein the combining is performed by coating the electrical device with a substance that contains the agent or the composition.

10526. The method of item 10300 wherein the combining is performed by coating the electrical device with a substance that absorbs the agent.

10527. The method of item 10300 wherein the combining is performed by interweaving the electrical device with a thread composed of, or coated with, the agent or the composition.

10528. The method of item 10300 wherein the combining is performed by completely covering the electrical device with a sleeve that contains the agent or the composition.

10529. The method of item 10300 wherein the combining is performed by covering a portion of the electrical device with a sleeve that contains the agent or the composition.

10530. The method of item 10300 wherein the combining is performed by completely covering the electrical device with a cover that contains the agent or the composition.

10531. The method of item 10300 wherein the combining is performed by covering a portion of the electrical device with a cover that contains the agent or the composition.

10532. The method of item 10300 wherein the combining is performed by completely covering the electrical device with an electrospun fabric that contains the agent or the composition.

10533. The method of item 10300 wherein the combining is performed by covering a portion of the electrical device with an electrospun fabric that contains the agent or the composition.

10534. The method of item 10300 wherein the combining is performed by completely covering the electrical device with a mesh that contains the agent or the composition.

10535. The method of item 10300 wherein the combining is performed by covering a portion of the electrical device with a mesh that contains the agent or the composition.

10536. The method of item 10300 wherein the combining is performed by constructing a portion of the electrical device with the agent or the composition.

10537. The method of item 10300 wherein the combining is performed by impregnating the electrical device with the agent or the composition.

10538. The method of item 10300 wherein the combining is performed by constructing a portion of the electrical device from a degradable polymer that releases the agent.

10539. The method of item 10300 wherein the combining is performed by dipping the electrical device into a solution that comprise the agent and an inert solvent for the electrical device.

10540. The method of item 10300 wherein the combining is performed by dipping the electrical device into a solution that comprises the agent and a solvent that will swell the electrical device.

10541. The method of item 10300 wherein the combining is performed by dipping the electrical device into a solution that comprises the agent and a solvent that will dissolve the electrical device.

10542. The method of item 10300 wherein the combining is performed by dipping the electrical device into a solution that comprises the agent, a polymer and an inert solvent for the electrical device.

10543. The method of item 10300 wherein the combining is performed by dipping the electrical device into a solution that comprises the agent, a polymer and a solvent that will swell the electrical device.

10544. The method of item 10300 wherein the combining is performed by dipping the electrical device into a solution that comprises the agent, a polymer and a solvent that will dissolve the electrical device.

10545. The method of item 10300 wherein the combining is performed by spraying the electrical device into a solution that comprises the agent and an inert solvent for the electrical device.

10546. The method of item 10300 wherein the combining is performed by spraying the electrical device into a solution that comprises the agent and a solvent that will swell the electrical device.

10547. The method of item 10300 wherein the combining is performed by spraying the electrical device into a solution that comprises the agent and a solvent that will dissolve the electrical device.

10548. The method of item 10300 wherein the combining is performed by spraying the electrical device into a solution that comprises the agent, a polymer and an inert solvent for the electrical device.

10549. The method of item 10300 wherein the combining is performed by spraying the electrical device into a solution that comprises the agent, a polymer and a solvent that will swell the electrical device.

10550. The method of item 10300 wherein the combining is performed by spraying the electrical device into a solution that comprises the agent, a polymer and a solvent that will dissolve the electrical device.

10551. The method for making a medical device of any one of items 10300-10550 wherein the vagus nerve stimulator is adapted for treating supraventricular arrhythmias.

10552. The method for making a medical device of any one of items 10300-10550 wherein the vagus nerve stimulator is adapted for treating angina pectoris.

10553. The method for making a medical device of any one of items 10300-10550 wherein the vagus nerve stimulator is adapted for treating atrial tachycardia.

10554. The method for making a medical device of any one of items 10300-10550 wherein the vagus nerve stimulator is adapted for treating atrial flutter.

10555. The method for making a medical device of any one of items 10300-10550 wherein the vagus nerve stimulator is adapted for treating arterial fibrillation.

10556. The method for making a medical device of any one of items 10300-10550 wherein the vagus nerve stimulator is arrhythmias that result in low cardiac output.

10557. The method for making a medical device of any one of items 10300-10550 wherein the vagus nerve stimulator comprises a programmable pulse generator.

10558. A method for making a medical device comprising: combining an electrical lead (i.e., an electrical device) and an anti-scarring agent or a composition comprising an anti-scarring agent, wherein the agent inhibits scarring between the device and a host into which the device is implanted.

10559. The method of item 10558 wherein the agent is an adensosine A2A receptor antagonist.

10560. The method of item 10558 wherein the agent is an AKT inhibitor.

10561. The method of item 10558 wherein the agent is an alpha 2 integrin antagonist, wherein the alpha 2 integrin antagonist is Pharmaprojects No. 5754 (Merck KgaA).

10562. The method of item 10558 wherein the agent is an alpha 4 integrin antagonist.

10563. The method of item 10558 wherein the agent is an alpha 7 nicotinic receptor agonist.

10564. The method of item 10558 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-11 55 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

10565. The method of item 10558 wherein the agent is an apoptosis antagonist.

10566. The method of item 10558 wherein the agent is an apoptosis activator.

10567. The method of item 10558 wherein the agent is a beta 1 integrin antagonist.

10568. The method of item 10558 wherein the agent is a beta tubulin inhibitor.

10569. The method of item 10558 wherein the agent is a blocker of enzyme production in Hepatitis C.

10570. The method of item 10558 wherein the agent is a Bruton's tyrosine kinase inhibitor.

10571. The method of item 10558 wherein the agent is a calcineurin inhibitor.

10572. The method of item 10558 wherein the agent is a caspase 3 inhibitor.

10573. The method of item 10558 wherein the agent is a CC chemokine receptor antagonist.

10574. The method of item 10558 wherein the agent is a cell cycle inhibitor selected from the group consisting of SNS-595 (Sunesis), synthadotin, KRX-0403, and an analogue or derivative thereof.

10575. The method of item 10558 wherein the agent is a cathepsin B inhibitor.

10576. The method of item 10558 wherein the agent is a cathepsin K inhibitor, wherein the cathepsin K inhibitor is 462795 (GlaxoSmithKline), INPL-022-D6 (Amura Therapeutics), or an analogue or derivative thereof.

10577. The method of item 10558 wherein the agent is a cathepsin L inhibitor.

10578. The method of item 10558 wherein the agent is a CD40 antagonist.

10579. The method of item 10558 wherein the agent is a chemokine receptor agonist.

10580. The method of item 10558 wherein the agent is a chymase inhibitor.

10581. The method of item 10558 wherein the agent is a collagenase antagonist.

10582. The method of item 10558 wherein the agent is a CXCR antagonist.

10583. The method of item 10558 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

10584. The method of item 10558 wherein the agent is a cyclooxygenase 1 inhibitor.

10585. The method of item 10558 wherein the agent is a DHFR inhibitor.

10586. The method of item 10558 wherein the agent is a dual integrin inhibitor.

10587. The method of item 10558 wherein the agent is an elastase inhibitor.

10588. The method of item 10558 wherein the agent is an elongation factor-1 alpha inhibitor.

10589. The method of item 10558 wherein the agent is an endothelial growth factor antagonist.

10590. The method of item 10558 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

10591. The method of item 10558 wherein the agent is an endotoxin antagonist.

10592. The method of item 10558 wherein the agent is an epothilone and tubulin binder.

10593. The method of item 10558 wherein the agent is an estrogen receptor antagonist

10594. The method of item 10558 wherein the agent is an FGF inhibitor.

10595. The method of item 10558 wherein the agent is a farnexyl transferase inhibitor.

10596. The method of item 10558 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

10597. The method of item 10558 wherein the agent is an FLT-3 kinase inhibitor.

10598. The method of item 10558 wherein the agent is an FGF receptor kinase inhibitor.

10599. The method of item 10558 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

10600. The method of item 10558 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

10601. The method of item 10558 wherein the agent is a histone deacetylase inhibitor.

10602. The method of item 10558 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

10603. The method of item 10558 wherein the agent is an ICAM inhibitor.

10604. The method of item 10558 wherein the agent is an IL, ICE and IRAK antagonist, wherein the antagonist is a CJ-14877, CP-424174 (Pfizer), NF-61 (Negma-Lerads), and an analogue or derivative thereof.

10605. The method of item 10558 wherein the agent is an IL-2 inhibitor.

10606. The method of item 10558 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

10607. The method of item 10558 wherein the agent is an IMPDH (inosine monophosphate).

10608. The method of item 10558 wherein the agent is an integrin antagonist.

10609. The method of item 10558 wherein the agent is an interleukin antagonist.

10610. The method of item 10558 wherein the agent is an inhibitor of type III receptor tyrosine kinase.

10611. The method of item 10558 wherein the agent is an irreversible inhibitor of enzyme methionine aminopeptidase type 2.

10612. The method of item 10558 wherein the agent is an isozyme selective delta protein kinase C inhibitor.

10613. The method of item 10558 wherein the agent a JAK3 enzyme inhibitor.

10614. The method of item 10558 wherein the agent is a JNK inhibitor.

10615. The method of item 10558 wherein the agent is a kinase inhibitor.

10616. The method of item 10558 wherein the agent is kinesin antagonist.

10617. The method of item 10558 wherein the agent is a kinesin antagonist.

10618. The method of item 10558 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

10619. The method of item 10558 wherein the agent is an MAP kinase inhibitor.

10620. The method of item 10558 wherein the agent is a matrix metalloproteinase inhibitor.

10621. The method of item 10558 wherein the agent is an MCP-CCR2 inhibitor.

10622. The method of item 10558 wherein the agent is an mTOR inhibitor.

10623. The method of item 10558 wherein the agent is an mTOR kinase inhibitor.

10624. The method of item 10558 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

10625. The method of item 10558 wherein the agent is an MIF inhibitor.

10626. The method of item 10558 wherein the agent is an MMP inhibitor.

10627. The method of item 10558 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

10628. The method of item 10558 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

10629. The method of item 10558 wherein the agent is a nitric oxide agonist.

10630. The method of item 10558 wherein the agent is an ornithine decarboxylase inhibitor.

10631. The method of item 10558 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

10632. The method of item 10558 wherein the agent is a palmitoyl-protein thioesterase inhibitor.

10633. The method of item 10558 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

10634. The method of item 10558 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 11102546-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

10635. The method of item 10558 wherein the agent is a phosphatase inhibitor.

10636. The method of item 10558 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

10637. The method of item 10558 wherein the agent is a PKC inhibitor.

10638. The method of item 10558 wherein the agent is a platelet activating factor antagonist.

10639. The method of item 10558 wherein the agent is a platelet-derived growth factor receptor kinase inhibitor.

10640. The method of item 10558 wherein the agent is a prolyl hydroxylase inhibitor.

10641. The method of item 10558 wherein the agent is a polymorphonuclear neutrophil inhibitor.

10642. The method of item 10558 wherein the agent is a protein kinase B inhibitor.

10643. The method of item 10558 wherein the agent is a protein kinase C stimulant.

10644. The method of item 10558 wherein the agent is a purine nucleoside analogue.

10645. The method of item 10558 wherein the agent is a purinoreceptor P2X antagonist.

10646. The method of item 10558 wherein the agent is a Raf kinase inhibitor.

10647. The method of item 10558 wherein the agent is a reversible inhibitor of ErbB1 and ErbB2.

10648. The method of item 10558 wherein the agent is a ribonucleoside triphosphate reductase inhibitor.

10649. The method of item 10558 wherein the agent is an SDF-1 antagonist.

10650. The method of item 10558 wherein the agent is a sheddase inhibitor.

10651. The method of item 10558 wherein the agent is an SRC inhibitor.

10652. The method of item 10558 wherein the agent is a stromelysin inhibitor.

10653. The method of item 10558 wherein the agent is an Syk kinase inhibitor.

10654. The method of item 10558 wherein the agent is a telomerase inhibitor.

10655. The method of item 10558 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

10656. The method of item 10558 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

10657. The method of item 10558 wherein the agent is a Toll receptor inhibitor.

10658. The method of item 10558 wherein the agent is a tubulin antagonist.

10659. The method of item 10558 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

10660. The method of item 10558 wherein the agent is a VEGF inhibitor.

10661. The method of item 10558 wherein the agent is a vitamin D receptor agonist.

10662. The method of item 10558 wherein the agent is ZD-6474 (an angiogenesis inhibitor).

10663. The method of item 10558 wherein the agent is AP-23573 (an mTOR inhibitor).

10664. The method of item 10558 wherein the agent is synthadotin (a tubulin antagonist).

10665. The method of item 10558 wherein the agent is S-0885 (a collagenase inhibitor).

10666. The method of item 10558 wherein the agent is aplidine (an elongation factor-1 alpha inhibitor).

10667. The method of item 10558 wherein the agent is ixabepilone (an epithilone).

10668. The method of item 10558 wherein the agent is IDN-5390 (an angiogenesis inhibitor).

10669. The method of item 10558 wherein the agent is SB-2723005 (an angiogenesis inhibitor).

10670. The method of item 10558 wherein the agent is ABT-518 (an angiogenesis inhibitor).

10671. The method of item 10558 wherein the agent is combretastatin (an angiogenesis inhibitor).

10672. The method of item 10558 wherein the agent is anecortave acetate (an angiogenesis inhibitor).

10673. The method of item 10558 wherein the agent is SB-715992 (a kinesin antagonist).

10674. The method of item 10558 wherein the agent is temsirolimus (an mTOR inhibitor).

10675. The method of item 10558 wherein the agent is adalimumab (a TNFα antagonist).

10676. The method of item 10558, wherein the composition comprises a polymer.

10677. The method of item 10558, wherein the composition comprises a polymeric carrier.

10678. The method of item 10558 wherein the anti-scarring agent inhibits adhesion between the medical device and a host into which the medical device is implanted.

10679. The method of item 10558 wherein the medical device delivers the anti-scarring agent locally to tissue proximate to the medical device.

10680. The method of item 10558 wherein the medical device has a coating that comprises the anti-scarring agent.

10681. The method of item 10558, wherein the medical device has a coating that comprises the agent and is disposed on a surface of the electrical device.

10682. The method of item 10558, wherein the medical device has a coating that comprises the agent and directly contacts the electrical device.

10683. The method of item 10558, wherein the medical device has a coating that comprises the agent and indirectly contacts the electrical device.

10684. The method of item 10558, wherein the medical device has a coating that comprises the agent and partially covers the electrical device.

10685. The method of item 10558, wherein the medical device has a coating that comprises the agent and completely covers the electrical device.

10686. The method of item 10558, wherein the medical device has a uniform coating.

10687. The method of item 10558, wherein the medical device has a non-uniform coating.

10688. The method of item 10558, wherein the medical device has a discontinuous coating.

10689. The method of item 10558, wherein the medical device has a patterned coating.

10690. The method of item 10558, wherein the medical device has a coating with a thickness of 100 μm or less.

10691. The method of item 10558, wherein the medical device has a coating with a thickness of 10 μm or less.

10692. The method of item 10558, wherein the medical device has a coating, and the coating adheres to the surface of the electrical device upon deployment of the electrical device.

10693. The method of item 10558, wherein the medical device has a coating, and wherein the coating is stable at room temperature for a period of 1 year.

10694. The method of item 10558, wherein the medical device has a coating, and wherein the anti-scarring agent is present in the coating in an amount ranging between about 0.0001% to about 1% by weight.

10695. The method of item 10558, wherein the medical device has a coating, and wherein the anti-scarring agent is present in the coating in an amount ranging between about 1% to about 10% by weight.

10696. The method of item 10558, wherein the medical device has a coating, and wherein the anti-scarring agent is present in the coating in an amount ranging between about 10% to about 25% by weight.

10697. The method of item 10558, wherein the medical device has a coating, and wherein the anti-scarring agent is present in the coating in an amount ranging between about 25% to about 70% by weight.

10698. The method of item 10558, wherein the medical device has a coating, and wherein the coating further comprises a polymer.

10699. The method of item 10558, wherein the medical device has a first coating having a first composition and a second coating having a second composition.

10700. The method of item 10558, wherein the medical device has a first coating having a first composition and a second coating having a second composition, wherein the first composition and the second composition are different.

10701. The method of item 10558, wherein the composition comprises a polymer.

10702. The method of item 10558, wherein the composition comprises a polymeric carrier.

10703. The method of item 10558, wherein the composition comprises a polymeric carrier, and wherein the polymeric carrier comprises a copolymer.

10704. The method of item 10558, wherein the composition comprises a polymeric carrier, and wherein the polymeric carrier comprises a block copolymer.

10705. The method of item 10558, wherein the composition comprises a polymeric carrier, and wherein the polymeric carrier comprises a random copolymer.

10706. The method of item 10558, wherein the composition comprises a polymeric carrier, and wherein the polymeric carrier comprises a biodegradable polymer.

10707. The method of item 10558, wherein the composition comprises a polymeric carrier, and wherein the polymeric carrier comprises a non-biodegradable polymer.

10708. The method of item 10558, wherein the composition comprises a polymeric carrier, and wherein the polymeric carrier comprises a hydrophilic polymer.

10709. The method of item 10558, wherein the composition comprises a polymeric carrier, and wherein the polymeric carrier comprises a hydrophobic polymer.

10710. The method of item 10558, wherein the composition comprises a polymeric carrier, and wherein the polymeric carrier comprises a polymer having hydrophilic domains.

10711. The method of item 10558, wherein the composition comprises a polymeric carrier, and wherein the polymeric carrier comprises a polymer having hydrophobic domains.

10712. The method of item 10558, wherein the composition comprises a polymeric carrier, and wherein the polymeric carrier comprises a non-conductive polymer.

10713. The method of item 10558, wherein the composition comprises a polymeric carrier, and wherein the polymeric carrier comprises an elastomer.

10714. The method of item 10558, wherein the composition comprises a polymeric carrier, and wherein the polymeric carrier comprises a hydrogel.

10715. The method of item 10558, wherein the composition comprises a polymeric carrier, and wherein the polymeric carrier comprises a silicone polymer.

10716. The method of item 10558, wherein the composition comprises a polymeric carrier, and wherein the polymeric carrier comprises a hydrocarbon polymer.

10717. The method of item 10558, wherein the composition comprises a polymeric carrier, and wherein the polymeric carrier comprises a styrene-derived polymer.

10718. The method of item 10558, wherein the composition comprises a polymeric carrier, and wherein the polymeric carrier comprises a butadiene polymer.

10719. The method of item 10558, wherein the composition comprises a polymeric carrier, and wherein the polymeric carrier comprises a macromer.

10720. The method of item 10558, wherein the composition comprises a polymeric carrier, and wherein the polymeric carrier comprises a poly(ethylene glycol) polymer.

10721. The method of item 10558 wherein the composition comprises a polymeric carrier, and wherein the polymeric carrier comprises an amorphous polymer.

10722. The method of item 10558, wherein the medical device comprises a lubricious coating.

10723. The method of item 10558 wherein the anti-scarring agent is located within pores or holes of the medical device.

10724. The method of item 10558 wherein the anti-scarring agent is located within a channel, lumen, or divet of the medical device.

10725. The method of item 10558, wherein the medical device further comprises a second pharmaceutically active agent.

10726. The method of item 10558 wherein the medical device further comprises an anti-inflammatory agent.

10727. The method of item 10558 wherein the medical device further comprises an agent that inhibits infection.

10728. The method of item 10558 wherein the medical device further comprises an agent that inhibits infection, and wherein the agent is an anthracycline.

10729. The method of item 10558 wherein the medical device further comprises an agent that inhibits infection, and wherein the agent is doxorubicin.

10730. The method of item 10558 wherein the medical device further comprises an agent that inhibits infection, and wherein the agent is mitoxantrone.

10731. The method of item 10558 wherein the medical device further comprises an agent that inhibits infection, and wherein the agent is a fluoropyrimidine.

10732. The method of item 10558 wherein the medical device further comprises an agent that inhibits infection, and wherein the agent is 5-fluorouracil (5-FU).

10733. The method of item 10558 wherein the medical device further comprises an agent that inhibits infection, and wherein the agent is a folic acid antagonist.

10734. The method of item 10558 wherein the medical device further comprises an agent that inhibits infection, and wherein the agent is methotrexate.

10735. The method of item 10558 wherein the medical device further comprises an agent that inhibits infection, and wherein the agent is a podophylotoxin.

10736. The method of item 10558 wherein the medical device further comprises an agent that inhibits infection, and wherein the agent is etoposide.

10737. The method of item 10558 wherein the medical device further comprises an agent that inhibits infection, and wherein the agent is a camptothecin.

10738. The method of item 10558 wherein the medical device further comprises an agent that inhibits infection, and wherein the agent is a hydroxyurea.

10739. The method of item 10558 wherein the medical device further comprises an agent that inhibits infection, and wherein the agent is a platinum complex.

10740. The method of item 10558 wherein the medical device further comprises an agent that inhibits infection, and wherein the agent is cisplatin.

10741. The method of item 10558 wherein the medical device further comprises an anti-thrombotic agent.

10742. The method of item 10558 wherein the medical device further comprises a visualization agent.

10743. The method of item 10558 wherein the medical device further comprises a visualization agent, wherein the visualization agent is a radiopaque material, and wherein the radiopaque material further comprises a metal, a halogenated compound, or a barium containing compound.

10744. The method of item 10558 wherein the medical device further comprises a visualization agent, wherein the visualization agent is a radiopaque material, and wherein the radiopaque material further comprises barium, tantalum, or technetium.

10745. The method of item 10558 wherein the medical device further comprises a visualization agent, and wherein the visualization agent is a MRI responsive material.

10746. The method of item 10558 wherein the medical device further comprises a visualization agent, and wherein the visualization agent further comprises a gadolinium chelate.

10747. The method of item 10558 wherein the medical device further comprises a visualization agent, and wherein the visualization agent further comprises iron, magnesium, manganese, copper, or chromium.

10748. The method of item 10558 wherein the medical device further comprises a visualization agent, and wherein the visualization agent further comprises an iron oxide compound.

10749. The method of item 10558 wherein the medical device further comprises a visualization agent, and wherein the visualization agent further comprises a dye, pigment, or colorant.

10750. The method of item 10558 wherein the medical device further comprises an echogenic material.

10751. The method of item 10558 wherein the medical device further comprises an echogenic material, and wherein the echogenic material is in the form of a coating.

10752. The method of item 10558 wherein the medical device is sterile.

10753. The method of item 10558 wherein the anti-scarring agent is released into tissue in the vicinity of the medical device after deployment of the medical device.

10754. The method of item 10558 wherein the anti-scarring agent is released into tissue in the vicinity of the medical device after deployment of the medical device, and wherein the tissue is connective tissue.

10755. The method of item 10558 wherein the anti-scarring agent is released into tissue in the vicinity of the medical device after deployment of the medical device, and wherein the tissue is muscle tissue.

10756. The method of item 10558 wherein the anti-scarring agent is released into tissue in the vicinity of the medical device after deployment of the medical device, and wherein the tissue is nerve tissue.

10757. The method of item 10558 wherein the anti-scarring agent is released into tissue in the vicinity of the medical device after deployment of the medical device, and wherein the tissue is epithelium tissue.

10758. The method of item 10558 wherein the anti-scarring agent is released in effective concentrations from the medical device over a period ranging from the time of deployment of the medical device to about 1 year.

10759. The method of item 10558 wherein the anti-scarring agent is released in effective concentrations from the medical device over a period ranging from about 1 month to 6 months.

10760. The method of item 10558 wherein the anti-scarring agent is released in effective concentrations from the medical device over a period ranging from about 1-90 days.

10761. The method of item 10558 wherein the anti-scarring agent is released in effective concentrations from the medical device at a constant rate.

10762. The method of item 10558 wherein the anti-scarring agent is released in effective concentrations from the medical device at an increasing rate.

10763. The method of item 10558 wherein the anti-scarring agent is released in effective concentrations from the medical device at a decreasing rate.

10764. The method of item 10558 wherein the anti-scarring agent is released in effective concentrations from the composition comprising the anti-scarring agent by diffusion over a period ranging from the time of deployment of the medical device to about 90 days.

10765. The method of item 10558 wherein the anti-scarring agent is released in effective concentrations from the composition comprising the anti-scarring agent by erosion of the composition over a period ranging from the time of deployment of the medical device to about 90 days.

10766. The method of item 10558 wherein the medical device comprises about 0.01 μg to about 10 μg of the anti-scarring agent.

10767. The method of item 10558 wherein the medical device comprises about 10 μg to about 10 mg of the anti-scarring agent.

10768. The method of item 10558 wherein the medical device comprises about 10 mg to about 250 mg of the anti-scarring agent.

10769. The method of item 10558 wherein the medical device comprises about 250 mg to about 1000 mg of the anti-scarring agent.

10770. The method of item 10558 wherein the medical device comprises about 1000 mg to about 2500 mg of the anti-scarring agent.

10771. The method of item 10558 wherein a surface of the medical device comprises less than 0.01 μg of the anti-scarring agent per mm² of medical device surface to which the anti-scarring agent is applied.

10772. The method of item 10558 wherein a surface of the medical device comprises about 0.01 μg to about 1 μg of the anti-scarring agent per mm² of medical device surface to which the anti-scarring agent is applied.

10773. The method of item 10558 wherein a surface of the medical device comprises about 1 μg to about 10 μg of the anti-scarring agent per mm² of medical device surface to which the anti-scarring agent is applied.

10774. The method of item 10558 wherein a surface of the medical device comprises about 10 μg to about 250 μg of the anti-scarring agent per mm² of medical device surface to which the anti-scarring agent is applied.

10775. The method of item 10558 wherein a surface of the medical device comprises about 250 μg to about 1000 μg of the anti-scarring agent of anti-scarring agent per mm² of medical device surface to which the anti-scarring agent is applied.

10776. The method of item 10558 wherein a surface of the medical device comprises about 1000 μg to about 2500 μg of the anti-scarring agent per mm² of medical device surface to which the anti-scarring agent is applied.

10777. The method of item 10558 wherein the combining is performed by direct affixing the agent or the composition to the electrical device.

10778. The method of item 10558 wherein the combining is performed by spraying the agent or the component onto the electrical device.

10779. The method of item 10558 wherein the combining is performed by electrospraying the agent or the composition onto the electrical device.

10780. The method of item 10558 wherein the combining is performed by dipping the electrical device into a solution comprising the agent or the composition.

10781. The method of item 10558 wherein the combining is performed by covalently attaching the agent or the composition to the electrical device.

10782. The method of item 10558 wherein the combining is performed by non-covalently attaching the agent or the composition to the electrical device.

10783. The method of item 10558 wherein the combining is performed by coating the electrical device with a substance that contains the agent or the composition.

10784. The method of item 10558 wherein the combining is performed by coating the electrical device with a substance that absorbs the agent.

10785. The method of item 10558 wherein the combining is performed by interweaving the electrical device with a thread composed of, or coated with, the agent or the composition.

10786. The method of item 10558 wherein the combining is performed by completely covering the electrical device with a sleeve that contains the agent or the composition.

10787. The method of item 10558 wherein the combining is performed by covering a portion of the electrical device with a sleeve that contains the agent or the composition.

10788. The method of item 10558 wherein the combining is performed by completely covering the electrical device with a cover that contains the agent or the composition.

10789. The method of item 10558 wherein the combining is performed by covering a portion of the electrical device with a cover that contains the agent or the composition.

10790. The method of item 10558 wherein the combining is performed by completely covering the electrical device with an electrospun fabric that contains the agent or the composition.

10791. The method of item 10558 wherein the combining is performed by covering a portion of the electrical device with an electrospun fabric that contains the agent or the composition.

10792. The method of item 10558 wherein the combining is performed by completely covering the electrical device with a mesh that contains the agent or the composition.

10793. The method of item 10558 wherein the combining is performed by covering a portion of the electrical device with a mesh that contains the agent or the composition.

10794. The method of item 10558 wherein the combining is performed by constructing a portion of the electrical device with the agent or the composition.

10795. The method of item 10558 wherein the combining is performed by impregnating the electrical device with the agent or the composition.

10796. The method of item 10558 wherein the combining is performed by constructing a portion of the electrical device from a degradable polymer that releases the agent.

10797. The method of item 10558 wherein the combining is performed by dipping the electrical device into a solution that comprise the agent and an inert solvent for the electrical device.

10798. The method of item 10558 wherein the combining is performed by dipping the electrical device into a solution that comprises the agent and a solvent that will swell the electrical device.

10799. The method of item 10558 wherein the combining is performed by dipping the electrical device into a solution that comprises the agent and a solvent that will dissolve the electrical device.

10800. The method of item 10558 wherein the combining is performed by dipping the electrical device into a solution that comprises the agent, a polymer and an inert solvent for the electrical device.

10801. The method of item 10558 wherein the combining is performed by dipping the electrical device into a solution that comprises the agent, a polymer and a solvent that will swell the electrical device.

10802. The method of item 10558 wherein the combining is performed by dipping the electrical device into a solution that comprises the agent, a polymer and a solvent that will dissolve the electrical device.

10803. The method of item 10558 wherein the combining is performed by spraying the electrical device into a solution that comprises the agent and an inert solvent for the electrical device.

10804. The method of item 10558 wherein the combining is performed by spraying the electrical device into a solution that comprises the agent and a solvent that will swell the electrical device.

10805. The method of item 10558 wherein the combining is performed by spraying the electrical device into a solution that comprises the agent and a solvent that will dissolve the electrical device.

10806. The method of item 10558 wherein the combining is performed by spraying the electrical device into a solution that comprises the agent, a polymer and an inert solvent for the electrical device.

10807. The method of item 10558 wherein the combining is performed by spraying the electrical device into a solution that comprises the agent, a polymer and a solvent that will swell the electrical device.

10808. The method of item 10558 wherein the combining is performed by spraying the electrical device into a solution that comprises the agent, a polymer and a solvent that will dissolve the electrical device.

10809. The method for making a medical device of any one of items 10558-10808 wherein the electrical lead comprises a connector assembly, a conductor and an electrode.

10810. The method for making a medical device of any one of items 10558-10808 wherein the electrical lead is unipolar.

10811. The method for making a medical device of any one of items 10558-10808 wherein the electrical lead is bipolar.

10812. The method for making a medical device of any one of items 10558-10808 wherein the electrical lead is tripolar.

10813. The method for making a medical device of any one of items 10558-10808 wherein the electrical lead is quadripolar.

10814. The method for making a medical device of any one of items 10558-10808 wherein the electrical lead comprises an insulating sheath.

10815. The method for making a medical device of any one of items 10558-10808 wherein the electrical lead is a medical lead.

10816. The method for making a medical device of any one of items 10558-10808 wherein the electrical lead is a cardiac lead.

10817. The method for making a medical device of any one of items 10558-10808 wherein the electrical lead is a pacer lead.

10818. The method for making a medical device of any one of items 10558-10808 wherein the electrical lead is a pacing lead.

10819. The method for making a medical device of any one of items 10558-10808 wherein the electrical lead is a pacemaker lead.

10820. The method for making a medical device of any one of items 10558-10808 wherein the electrical lead is an endocardial lead.

10821. The method for making a medical device of any one of items 10558-10808 wherein the electrical lead is an endocardial pacing lead.

10822. The method for making a medical device of any one of items 10558-10808 wherein the electrical lead is a cardioversion lead.

10823. The method for making a medical device of any one of items 10558-10808 wherein the electrical lead is an epicardial lead.

10824. The method for making a medical device of any one of items 10558-10808 wherein the electrical lead is an epicardial defibrillator lead.

10825. The method for making a medical device of any one of items 10558-10808 wherein the electrical lead is a patch defibrillator.

10826. The method for making a medical device of any one of items 10558-10808 wherein the electrical lead is a patch lead.

10827. The method for making a medical device of any one of items 10558-10808 wherein the electrical lead is an electrical patch.

10828. The method for making a medical device of any one of items 10558-10808 wherein the electrical lead is a transvenous lead.

10829. The method for making a medical device of any one of items 10558-10808 wherein the electrical lead is an active fixation lead.

10830. The method for making a medical device of any one of items 10558-10808 wherein the electrical lead is a passive fixation lead.

10831. The method for making a medical device of any one of items 10558-10808 wherein the electrical lead is a sensing lead.

10832. The method for making a medical device of any one of items 10558-10808 wherein the electrical lead is expandable.

10833. The method for making a medical device of any one of items 10558-10808 wherein the electrical lead has a coil configuration.

10834. The method for making a medical device of any one of items 10558-10808 wherein the electrical lead has an active fixation element for attachment to host tissue.

10835. A method for making a medical device comprising: combining a neurostimulator (i.e., an electrical device) and an anti-scarring agent or a composition comprising an anti-scarring agent, wherein the agent inhibits scarring between the device and a host into which the device is implanted.

10836. The method of item 10835 wherein the agent is an adensosine A2A receptor antagonist.

10837. The method of item 10835 wherein the agent is an AKT inhibitor.

10838. The method of item 10835 wherein the agent is an alpha 2 integrin antagonist, wherein the alpha 2 integrin antagonist is Pharmaprojects No. 5754 (Merck KgaA).

10839. The method of item 10835 wherein the agent is an alpha 4 integrin antagonist.

10840. The method of item 10835 wherein the agent is an alpha 7 nicotinic receptor agonist

10841. The method of item 10835 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

10842. The method of item 10835 wherein the agent is an apoptosis antagonist.

10843. The method of item 10835 wherein the agent is an apoptosis activator.

10844. The method of item 10835 wherein the agent is a beta 1 integrin antagonist.

10845. The method of item 10835 wherein the agent is a beta tubulin inhibitor.

10846. The method of item 10835 wherein the agent is a blocker of enzyme production in Hepatitis C.

10847. The method of item 10835 wherein the agent is a Bruton's tyrosine kinase inhibitor.

10848. The method of item 10835 wherein the agent is a calcineurin inhibitor.

10849. The method of item 10835 wherein the agent is a caspase 3 inhibitor.

10850. The method of item 10835 wherein the agent is a CC chemokine receptor antagonist.

10851. The method of item 10835 wherein the agent is a cell cycle inhibitor selected from the group consisting of SNS-595 (Sunesis), synthadotin, KRX-0403, and an analogue or derivative thereof.

10852. The method of item 10835 wherein the agent is a cathepsin B inhibitor.

10853. The method of item 10835 wherein the agent is a cathepsin K inhibitor, wherein the cathepsin K inhibitor is 462795 (GlaxoSmithKline), INPL-022-D6 (Amura Therapeutics), or an analogue or derivative thereof.

10854. The method of item 10835 wherein the agent is a cathepsin L inhibitor.

10855. The method of item 10835 wherein the agent is a CD40 antagonist.

10856. The method of item 10835 wherein the agent is a chemokine receptor agonist.

10857. The method of item 10835 wherein the agent is a chymase inhibitor.

10858. The method of item 10835 wherein the agent is a collagenase antagonist.

10859. The method of item 10835 wherein the agent is a CXCR antagonist.

10860. The method of item 10835 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

10861. The method of item 10835 wherein the agent is a cyclooxygenase 1 inhibitor.

10862. The method of item 10835 wherein the agent is a DHFR inhibitor.

10863. The method of item 10835 wherein the agent is a dual integrin inhibitor.

10864. The method of item 10835 wherein the agent is an elastase inhibitor.

10865. The method of item 10835 wherein the agent is an elongation factor-1 alpha inhibitor.

10866. The method of item 10835 wherein the agent is an endothelial growth factor antagonist.

10867. The method of item 10835 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

10868. The method of item 10835 wherein the agent is an endotoxin antagonist.

10869. The method of item 10835 wherein the agent is an epothilone and tubulin binder.

10870. The method of item 10835 wherein the agent is an estrogen receptor antagonist.

10871. The method of item 10835 wherein the agent is an FGF inhibitor.

10872. The method of item 10835 wherein the agent is a farnexyl transferase inhibitor.

10873. The method of item 10835 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

10874. The method of item 10835 wherein the agent is an FLT-3 kinase inhibitor.

10875. The method of item 10835 wherein the agent is an FGF receptor kinase inhibitor.

10876. The method of item 10835 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

10877. The method of item 10835 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

10878. The method of item 10835 wherein the agent is a histone deacetylase inhibitor.

10879. The method of item 10835 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

10880. The method of item 10835 wherein the agent is an ICAM inhibitor.

10881. The method of item 10835 wherein the agent is an IL, ICE and IRAK antagonist, wherein the antagonist is a CJ-14877, CP-424174 (Pfizer), NF-61 (Negma-Lerads), and an analogue or derivative thereof.

10882. The method of item 10835 wherein the agent is an IL-2 inhibitor.

10883. The method of item 10835 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

10884. The method of item 10835 wherein the agent is an IMPDH (inosine monophosphate).

10885. The method of item 10835 wherein the agent is an integrin antagonist.

10886. The method of item 10835 wherein the agent is an interleukin antagonist.

10887. The method of item 10835 wherein the agent is an inhibitor of type III receptor tyrosine kinase.

10888. The method of item 10835 wherein the agent is an irreversible inhibitor of enzyme methionine aminopeptidase type 2.

10889. The method of item 10835 wherein the agent is an isozyme selective delta protein kinase C inhibitor.

10890. The method of item 10835 wherein the agent a JAK3 enzyme inhibitor.

10891. The method of item 10835 wherein the agent is a JNK inhibitor.

10892. The method of item 10835 wherein the agent is a kinase inhibitor.

10893. The method of item 10835 wherein the agent is kinesin antagonist.

10894. The method of item 10835 wherein the agent is a kinesin antagonist.

10895. The method of item 10835 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

10896. The method of item 10835 wherein the agent is an MAP kinase inhibitor.

10897. The method of item 10835 wherein the agent is a matrix metalloproteinase inhibitor.

10898. The method of item 10835 wherein the agent is an MCP-CCR2 inhibitor.

10899. The method of item 10835 wherein the agent is an mTOR inhibitor.

10900. The method of item 10835 wherein the agent is an mTOR kinase inhibitor.

10901. The method of item 10835 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

10902. The method of item 10835 wherein the agent is an MIF inhibitor.

10903. The method of item 10835 wherein the agent is an MMP inhibitor.

10904. The method of item 10835 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

10905. The method of item 10835 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

10906. The method of item 10835 wherein the agent is a nitric oxide agonist.

10907. The method of item 10835 wherein the agent is an ornithine decarboxylase inhibitor.

10908. The method of item 10835 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

10909. The method of item 10835 wherein the agent is a palmitoyl-protein thioesterase inhibitor.

10910. The method of item 10835 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

10911. The method of item 10835 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

10912. The method of item 10835 wherein the agent is a phosphatase inhibitor.

10913. The method of item 10835 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

10914. The method of item 10835 wherein the agent is a PKC inhibitor.

10915. The method of item 10835 wherein the agent is a platelet activating factor antagonist.

10916. The method of item 10835 wherein the agent is a platelet-derived growth factor receptor kinase inhibitor.

10917. The method of item 10835 wherein the agent is a prolyl hydroxylase inhibitor.

10918. The method of item 10835 wherein the agent is a polymorphonuclear neutrophil inhibitor.

10919. The method of item 10835 wherein the agent is a protein kinase B inhibitor.

10920. The method of item 10835 wherein the agent is a protein kinase C stimulant.

10921. The method of item 10835 wherein the agent is a purine nucleoside analogue.

10922. The method of item 10835 wherein the agent is a purinoreceptor P2X antagonist.

10923. The method of item 10835 wherein the agent is a Raf kinase inhibitor.

10924. The method of item 10835 wherein the agent is a reversible inhibitor of ErbB1 and ErbB2.

10925. The method of item 10835 wherein the agent is a ribonucleoside triphosphate reductase inhibitor.

10926. The method of item 10835 wherein the agent is an SDF-1 antagonist.

10927. The method of item 10835 wherein the agent is a sheddase inhibitor.

10928. The method of item 10835 wherein the agent is an SRC inhibitor.

10929. The method of item 10835 wherein the agent is a stromelysin inhibitor.

10930. The method of item 10835 wherein the agent is an Syk kinase inhibitor.

10931. The method of item 10835 wherein the agent is a telomerase inhibitor.

10932. The method of item 10835 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

10933. The method of item 10835 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

10934. The method of item 10835 wherein the agent is a Toll receptor inhibitor.

10935. The method of item 10835 wherein the agent is a tubulin antagonist.

10936. The method of item 10835 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

10937. The method of item 10835 wherein the agent is a VEGF inhibitor.

10938. The method of item 10835 wherein the agent is a vitamin D receptor agonist.

10939. The method of item 10835 wherein the agent is ZD-6474 (an angiogenesis inhibitor).

10940. The method of item 10835 wherein the agent is AP-23573 (an mTOR inhibitor).

10941. The method of item 10835 wherein the agent is synthadotin (a tubulin antagonist).

10942. The method of item 10835 wherein the agent is S-0885 (a collagenase inhibitor).

10943. The method of item 10835 wherein the agent is aplidine (an elongation factor-1 alpha inhibitor).

10944. The method of item 10835 wherein the agent is ixabepilone (an epithilone).

10945. The method of item 10835 wherein the agent is IDN-5390 (an angiogenesis inhibitor).

10946. The method of item 10835 wherein the agent is SB-2723005 (an angiogenesis inhibitor).

10947. The method of item 10835 wherein the agent is ABT-518 (an angiogenesis inhibitor).

10948. The method of item 10835 wherein the agent is combretastatin (an angiogenesis inhibitor).

10949. The method of item 10835 wherein the agent is anecortave acetate (an angiogenesis inhibitor).

10950. The method of item 10835 wherein the agent is SB-715992 (a kinesin antagonist).

10951. The method of item 10835 wherein the agent is temsirolimus (an mTOR inhibitor).

10952. The method of item 10835 wherein the agent is adalimumab (a TNFα antagonist).

10953. The method of item 10835, wherein the composition comprises a polymer.

10954. The method of item 10835, wherein the composition comprises a polymeric carrier.

10955. The method of item 10835 wherein the anti-scarring agent inhibits adhesion between the medical device and a host into which the medical device is implanted.

10956. The method of item 10835 wherein the medical device delivers the anti-scarring agent locally to tissue proximate to the medical device.

10957. The method of item 10835 wherein the medical device has a coating that comprises the anti-scarring agent.

10958. The method of item 10835, wherein the medical device has a coating that comprises the agent and is disposed on a surface of the electrical device.

10959. The method of item 10835, wherein the medical device has a coating that comprises the agent and directly contacts the electrical device.

10960. The method of item 10835, wherein the medical device has a coating that comprises the agent and indirectly contacts the electrical device.

10961. The method of item 10835, wherein the medical device has a coating that comprises the agent and partially covers the electrical device.

10962. The method of item 10835, wherein the medical device has a coating that comprises the agent and completely covers the electrical device.

10963. The method of item 10835, wherein the medical device has a uniform coating.

10964. The method of item 10835, wherein the medical device has a non-uniform coating.

10965. The method of item 10835, wherein the medical device has a discontinuous coating.

10966. The method of item 10835, wherein the medical device has a patterned coating.

10967. The method of item 10835, wherein the medical device has a coating with a thickness of 100 μm or less.

10968. The method of item 10835, wherein the medical device has a coating with a thickness of 10 pm or less.

10969. The method of item 10835, wherein the medical device has a coating, and the coating adheres to the surface of the electrical device upon deployment of the electrical device.

10970. The method of item 10835, wherein the medical device has a coating, and wherein the coating is stable at room temperature for a period of 1 year.

10971. The method of item 10835, wherein the medical device has a coating, and wherein the anti-scarring agent is present in the coating in an amount ranging between about 0.0001% to about 1% by weight.

10972. The method of item 10835, wherein the medical device has a coating, and wherein the anti-scarring agent is present in the coating in an amount ranging between about 1% to about 10% by weight.

10973. The method of item 10835, wherein the medical device has a coating, and wherein the anti-scarring agent is present in the coating in an amount ranging between about 10% to about 25% by weight.

10974. The method of item 10835, wherein the medical device has a coating, and wherein the anti-scarring agent is present in the coating in an amount ranging between about 25% to about 70% by weight.

10975. The method of item 10835, wherein the medical device has a coating, and wherein the coating further comprises a polymer.

10976. The method of item 10835, wherein the medical device has a first coating having a first composition and a second coating having a second composition.

10977. The method of item 10835, wherein the medical device has a first coating having a first composition and a second coating having a second composition, wherein the first composition and the second composition are different.

10978. The method of item 10835, wherein the composition comprises a polymer.

10979. The method of item 10835, wherein the composition comprises a polymeric carrier.

10980. The method of item 10835, wherein the composition comprises a polymeric carrier, and wherein the polymeric carrier comprises a copolymer.

10981. The method of item 10835, wherein the composition comprises a polymeric carrier, and wherein the polymeric carrier comprises a block copolymer.

10982. The method of item 10835, wherein the composition comprises a polymeric carrier, and wherein the polymeric carrier comprises a random copolymer.

10983. The method of item 10835, wherein the composition comprises a polymeric carrier, and wherein the polymeric carrier comprises a biodegradable polymer.

10984. The method of item 10835, wherein the composition comprises a polymeric carrier, and wherein the polymeric carrier comprises a non-biodegradable polymer.

10985. The method of item 10835, wherein the composition comprises a polymeric carrier, and wherein the polymeric carrier comprises a hydrophilic polymer.

10986. The method of item 10835, wherein the composition comprises a polymeric carrier, and wherein the polymeric carrier comprises a hydrophobic polymer.

10987. The method of item 10835, wherein the composition comprises a polymeric carrier, and wherein the polymeric carrier comprises a polymer having hydrophilic domains.

10988. The method of item 10835, wherein the composition comprises a polymeric carrier, and wherein the polymeric carrier comprises a polymer having hydrophobic domains.

10989. The method of item 10835, wherein the composition comprises a polymeric carrier, and wherein the polymeric carrier comprises a non-conductive polymer.

10990. The method of item 10835, wherein the composition comprises a polymeric carrier, and wherein the polymeric carrier comprises an elastomer.

10991. The method of item 10835, wherein the composition comprises a polymeric carrier, and wherein the polymeric carrier comprises a hydrogel.

10992. The method of item 10835, wherein the composition comprises a polymeric carrier, and wherein the polymeric carrier comprises a silicone polymer.

10993. The method of item 10835, wherein the composition comprises a polymeric carrier, and wherein the polymeric carrier comprises a hydrocarbon polymer.

10994. The method of item 10835, wherein the composition comprises a polymeric carrier, and wherein the polymeric carrier comprises a styrene-derived polymer.

10995. The method of item 10835, wherein the composition comprises a polymeric carrier, and wherein the polymeric carrier comprises a butadiene polymer.

10996. The method of item 10835, wherein the composition comprises a polymeric carrier, and wherein the polymeric carrier comprises a macromer.

10997. The method of item 10835, wherein the composition comprises a polymeric carrier, and wherein the polymeric carrier comprises a poly(ethylene glycol) polymer.

10998. The method of item 10835 wherein the composition comprises a polymeric carrier, and wherein the polymeric carrier comprises an amorphous polymer.

10999. The method of item 10835, wherein the medical device comprises a lubricious coating.

11000. The method of item 10835 wherein the anti-scarring agent is located within pores or holes of the medical device.

11001. The method of item 10835 wherein the anti-scarring agent is located within a channel, lumen, or divet of the medical device.

11002. The method of item 10835, wherein the medical device further comprises a second pharmaceutically active agent.

11003. The method of item 10835 wherein the medical device further comprises an anti-inflammatory agent.

11004. The method of item 10835 wherein the medical device further comprises an agent that inhibits infection.

11005. The method of item 10835 wherein the medical device further comprises an agent that inhibits infection, and wherein the agent is an anthracycline.

11006. The method of item 10835 wherein the medical device further comprises an agent that inhibits infection, and wherein the agent is doxorubicin.

11007. The method of item 10835 wherein the medical device further comprises an agent that inhibits infection, and wherein the agent is mitoxantrone.

11008. The method of item 10835 wherein the medical device further comprises an agent that inhibits infection, and wherein the agent is a fluoropyrimidine.

11009. The method of item 10835 wherein the medical device further comprises an agent that inhibits infection, and wherein the agent is 5-fluorouracil (5-FU).

11010. The method of item 10835 wherein the medical device further comprises an agent that inhibits infection, and wherein the agent is a folic acid antagonist.

11011. The method of item 10835 wherein the medical device further comprises an agent that inhibits infection, and wherein the agent is methotrexate.

11012. The method of item 10835 wherein the medical device further comprises an agent that inhibits infection, and wherein the agent is a podophylotoxin.

11013. The method of item 10835 wherein the medical device further comprises an agent that inhibits infection, and wherein the agent is etoposide.

11014. The method of item 10835 wherein the medical device further comprises an agent that inhibits infection, and wherein the agent is a camptothecin.

11015. The method of item 10835 wherein the medical device further comprises an agent that inhibits infection, and wherein the agent is a hydroxyurea.

11016. The method of item 10835 wherein the medical device further comprises an agent that inhibits infection, and wherein the agent is a platinum complex.

11017. The method of item 10835 wherein the medical device further comprises an agent that inhibits infection, and wherein the agent is cisplatin.

11018. The method of item 10835 wherein the medical device further comprises an anti-thrombotic agent.

11019. The method of item 10835 wherein the medical device further comprises a visualization agent.

11020. The method of item 10835 wherein the medical device further comprises a visualization agent, wherein the visualization agent is a radiopaque material, and wherein the radiopaque material further comprises a metal, a halogenated compound, or a barium containing compound.

11021. The method of item 10835 wherein the medical device further comprises a visualization agent, wherein the visualization agent is a radiopaque material, and wherein the radiopaque material further comprises barium, tantalum, or technetium.

11022. The method of item 10835 wherein the medical device further comprises a visualization agent, and wherein the visualization agent is a MRI responsive material.

11023. The method of item 10835 wherein the medical device further comprises a visualization agent, and wherein the visualization agent further comprises a gadolinium chelate.

11024. The method of item 10835 wherein the medical device further comprises a visualization agent, and wherein the visualization agent further comprises iron, magnesium, manganese, copper, or chromium.

11025. The method of item 10835 wherein the medical device further comprises a visualization agent, and wherein the visualization agent further comprises an iron oxide compound.

11026. The method of item 10835 wherein the medical device further comprises a visualization agent, and wherein the visualization agent further comprises a dye, pigment, or colorant.

11027. The method of item 10835 wherein the medical device further comprises an echogenic material.

11028. The method of item 10835 wherein the medical device further comprises an echogenic material, and wherein the echogenic material is in the form of a coating.

11029. The method of item 10835 wherein the medical device is sterile.

11030. The method of item 10835 wherein the anti-scarring agent is released into tissue in the vicinity of the medical device after deployment of the medical device.

11031. The method of item 10835 wherein the anti-scarring agent is released into tissue in the vicinity of the medical device after deployment of the medical device, and wherein the tissue is connective tissue.

11032. The method of item 10835 wherein the anti-scarring agent is released into tissue in the vicinity of the medical device after deployment of the medical device, and wherein the tissue is muscle tissue.

11033. The method of item 10835 wherein the anti-scarring agent is released into tissue in the vicinity of the medical device after deployment of the medical device, and wherein the tissue is nerve tissue.

11034. The method of item 10835 wherein the anti-scarring agent is released into tissue in the vicinity of the medical device after deployment of the medical device, and wherein the tissue is epithelium tissue.

11035. The method of item 10835 wherein the anti-scarring agent is released in effective concentrations from the medical device over a period ranging from the time of deployment of the medical device to about 1 year.

11036. The method of item 10835 wherein the anti-scarring agent is released in effective concentrations from the medical device over a period ranging from about 1 month to 6 months.

11037. The method of item 10835 wherein the anti-scarring agent is released in effective concentrations from the medical device over a period ranging from about 1-90 days.

11038. The method of item 10835 wherein the anti-scarring agent is released in effective concentrations from the medical device at a constant rate.

11039. The method of item 10835 wherein the anti-scarring agent is released in effective concentrations from the medical device at an increasing rate.

11040. The method of item 10835 wherein the anti-scarring agent is released in effective concentrations from the medical device at a decreasing rate.

11041. The method of item 10835 wherein the anti-scarring agent is released in effective concentrations from the composition comprising the anti-scarring agent by diffusion over a period ranging from the time of deployment of the medical device to about 90 days.

11042. The method of item 10835 wherein the anti-scarring agent is released in effective concentrations from the composition comprising the anti-scarring agent by erosion of the composition over a period ranging from the time of deployment of the medical device to about 90 days.

11043. The method of item 10835 wherein the medical device comprises about 0.01 μg to about 10 μg of the anti-scarring agent.

11044. The method of item 10835 wherein the medical device comprises about 10 μg to about 10 mg of the anti-scarring agent.

11045. The method of item 10835 wherein the medical device comprises about 10 mg to about 250 mg of the anti-scarring agent.

11046. The method of item 10835 wherein the medical device comprises about 250 mg to about 1000 mg of the anti-scarring agent.

11047. The method of item 10835 wherein the medical device comprises about 1000 mg to about 2500 mg of the anti-scarring agent.

11048. The method of item 10835 wherein a surface of the medical device comprises less than 0.01 μg of the anti-scarring agent per mm² of medical device surface to which the anti-scarring agent is applied.

11049. The method of item 10835 wherein a surface of the medical device comprises about 0.01 μg to about 1 μg of the anti-scarring agent per mm² of medical device surface to which the anti-scarring agent is applied.

11050. The method of item 10835 wherein a surface of the medical device comprises about 1 μg to about 10 μg of the anti-scarring agent per mm² of medical device surface to which the anti-scarring agent is applied.

11051. The method of item 10835 wherein a surface of the medical device comprises about 10 μg to about 250 μg of the anti-scarring agent per mm² of medical device surface to which the anti-scarring agent is applied.

11052. The method of item 10835 wherein a surface of the medical device comprises about 250 μg to about 1000 μg of the anti-scarring agent of anti-scarring agent per mm² of medical device surface to which the anti-scarring agent is applied.

11053. The method of item 10835 wherein a surface of the medical device comprises about 1000 μg to about 2500 μg of the anti-scarring agent per mm² of medical device surface to which the anti-scarring agent is applied.

11054. The method of item 10835 wherein the combining is performed by direct affixing the agent or the composition to the electrical device.

11055. The method of item 10835 wherein the combining is performed by spraying the agent or the component onto the electrical device.

11056. The method of item 10835 wherein the combining is performed by electrospraying the agent or the composition onto the electrical device.

11057. The method of item 10835 wherein the combining is performed by dipping the electrical device into a solution comprising the agent or the composition.

11058. The method of item 10835 wherein the combining is performed by covalently attaching the agent or the composition to the electrical device.

11059. The method of item 10835 wherein the combining is performed by non-covalently attaching the agent or the composition to the electrical device.

11060. The method of item 10835 wherein the combining is performed by coating the electrical device with a substance that contains the agent or the composition.

11061. The method of item 10835 wherein the combining is performed by coating the electrical device with a substance that absorbs the agent.

11062. The method of item 10835 wherein the combining is performed by interweaving the electrical device with a thread composed of, or coated with, the agent or the composition.

11063. The method of item 10835 wherein the combining is performed by completely covering the electrical device with a sleeve that contains the agent or the composition.

11064. The method of item 10835 wherein the combining is performed by covering a portion of the electrical device with a sleeve that contains the agent or the composition.

11065. The method of item 10835 wherein the combining is performed by completely covering the electrical device with a cover that contains the agent or the composition.

11066. The method of item 10835 wherein the combining is performed by covering a portion of the electrical device with a cover that contains the agent or the composition.

11067. The method of item 10835 wherein the combining is performed by completely covering the electrical device with an electrospun fabric that contains the agent or the composition.

11068. The method of item 10835 wherein the combining is performed by covering a portion of the electrical device with an electrospun fabric that contains the agent or the composition.

11069. The method of item 10835 wherein the combining is performed by completely covering the electrical device with a mesh that contains the agent or the composition.

11070. The method of item 10835 wherein the combining is performed by covering a portion of the electrical device with a mesh that contains the agent or the composition.

11071. The method of item 10835 wherein the combining is performed by constructing a portion of the electrical device with the agent or the composition.

11072. The method of item 10835 wherein the combining is performed by impregnating the electrical device with the agent or the composition.

11073. The method of item 10835 wherein the combining is performed by constructing a portion of the electrical device from a degradable polymer that releases the agent.

11074. The method of item 10835 wherein the combining is performed by dipping the electrical device into a solution that comprise the agent and an inert solvent for the electrical device.

11075. The method of item 10835 wherein the combining is performed by dipping the electrical device into a solution that comprises the agent and a solvent that will swell the electrical device.

11076. The method of item 10835 wherein the combining is performed by dipping the electrical device into a solution that comprises the agent and a solvent that will dissolve the electrical device.

11077. The method of item 10835 wherein the combining is performed by dipping the electrical device into a solution that comprises the agent, a polymer and an inert solvent for the electrical device.

11078. The method of item 10835 wherein the combining is performed by dipping the electrical device into a solution that comprises the agent, a polymer and a solvent that will swell the electrical device.

11079. The method of item 10835 wherein the combining is performed by dipping the electrical device into a solution that comprises the agent, a polymer and a solvent that will dissolve the electrical device.

11080. The method of item 10835 wherein the combining is performed by spraying the electrical device into a solution that comprises the agent and an inert solvent for the electrical device.

11081. The method of item 10835 wherein the combining is performed by spraying the electrical device into a solution that comprises the agent and a solvent that will swell the electrical device.

11082. The method of item 10835 wherein the combining is performed by spraying the electrical device into a solution that comprises the agent and a solvent that will dissolve the electrical device.

11083. The method of item 10835 wherein the combining is performed by spraying the electrical device into a solution that comprises the agent, a polymer and an inert solvent for the electrical device.

11084. The method of item 10835 wherein the combining is performed by spraying the electrical device into a solution that comprises the agent, a polymer and a solvent that will swell the electrical device.

11085. The method of item 10835 wherein the combining is performed by spraying the electrical device into a solution that comprises the agent, a polymer and a solvent that will dissolve the electrical device.

11086. The method for making a medical device of any one of items 10835-11085 wherein the neurostimulator is a spinal cord stimulator.

11087. The method for making a medical device of any one of items 10835-11085 wherein the neurostimulator is a brain stimulator.

11088. The method for making a medical device of any one of items 10835-11085 wherein the neurostimulator is a vagus nerve stimulator.

11089. The method for making a medical device of any one of items 10835-11085 wherein the neurostimulator is a sacral nerve stimulator.

11090. The method for making a medical device of any one of items 10835-11085 wherein the neurostimulator is a gastric nerve stimulator.

11091. The method for making a medical device of any one of items 10835-11085 wherein the neurostimulator is an auditory nerve stimulator.

11092. The method for making a medical device of any one of items 10835-11085 wherein the neurostimulator delivers stimulation to organs.

11093. The method for making a medical device of any one of items 10835-11085 wherein the neurostimulator delivers stimulation to bone.

11094. The method for making a medical device of any one of items 10835-11085 wherein the neurostimulator delivers stimulation to muscles.

11095. The method for making a medical device of any one of items 10835-11085 wherein the neurostimulator delivers stimulation to tissues.

11096. The method for making a medical device of any one of items 10835-11085 wherein the neurostimulator is a device for continuous subarachnoid infusion.

11097. The method for making a medical device of any one of items 10835-11085 wherein the neurostimulator is an implantable electrode.

11098. The method for making a medical device of any one of items 10835-11085 wherein the neurostimulator is an electrical lead.

11099. The method for making a medical device of any one of items 10835-11085 wherein the neurostimulator is a simulation catheter lead.

11100. The method for making a medical device of any one of items 10835-11085 wherein the neurostimulator is cochlear implant.

11101. The method for making a medical device of any one of items 10835-11085 wherein the neurostimulator is a microstimulator.

11102. The method for making a medical device of any one of items 10835-11085 wherein the neurostimulator is battery powered.

11103. The method for making a medical device of any one of items 10835-11085 wherein the neurostimulator is radio frequency powered.

11104. The method for making a medical device of any one of items 10835-11085 wherein the neurostimulator is both battery and radio frequency powered.

11105. The method for making a medical device of any one of items 10835-11085 wherein the neurostimulator is adapted for treating or preventing pain.

11106. The method for making a medical device of any one of items 10835-11085 wherein the neurostimulator is adapted for treating or preventing epilepsy.

11107. The method for making a medical device of any one of items 10835-11085 wherein the neurostimulator is adapted for treating or preventing Parkinson's disease.

11108. The method for making a medical device of any one of items 10835-11085 wherein the neurostimulator is adapted for treating or preventing movement disorders.

11109. The method for making a medical device of any one of items 10835-11085 wherein the neurostimulator is adapted for treating or preventing obesity.

11110. The method for making a medical device of any one of items 10835-11085 wherein the neurostimulator is adapted for treating or preventing depression.

11111. The method for making a medical device of any one of items 10835-11085 wherein the neurostimulator is adapted for treating or preventing anxiety.

11112. The method for making a medical device of any one of items 10835-11085 wherein the neurostimulator is adapted for treating or preventing hearing loss.

11113. The method for making a medical device of any one of items 10835-11085 wherein the neurostimulator is adapted for treating or preventing ulcers.

11114. The method for making a medical device of any one of items 10835-11085 wherein the neurostimulator is adapted for treating or preventing deep vein thrombosis.

11115. The method for making a medical device of any one of items 10835-11085 wherein the neurostimulator is adapted for treating or preventing muscular atrophy.

11116. The method for making a medical device of any one of items 10835-11085 wherein the neurostimulator is adapted for treating or preventing joint stiffness.

11117. The method for making a medical device of any one of items 10835-11085 wherein the neurostimulator is adapted for treating or preventing muscle spasms.

11118. The method for making a medical device of any one of items 10835-11085 wherein the neurostimulator is adapted for treating or preventing osteoporosis.

11119. The method for making a medical device of any one of items 10835-11085 wherein the neurostimulator is adapted for treating or preventing scoliosis.

11120. The method for making a medical device of any one of items 10835-11085 wherein the neurostimulator is adapted for treating or preventing spinal disc degeneration.

11121. The method for making a medical device of any one of items 10835-11085 wherein the neurostimulator is adapted for treating or preventing spinal cord injury.

11122. The method for making a medical device of any one of items 10835-11085 wherein the neurostimulator is adapted for treating or preventing urinary dysfunction.

11123. The method for making a medical device of any one of items 10835-11085 wherein the neurostimulator is adapted for treating or preventing gastroparesis.

11124. The method for making a medical device of any one of items 10835-11085 wherein the neurostimulator is adapted for treating or preventing malignancy.

11125. The method for making a medical device of any one of items 10835-11085 wherein the neurostimulator is adapted for treating or preventing arachnoiditis.

11126. The method for making a medical device of any one of items 10835-11085 wherein the neurostimulator is adapted for treating or preventing chronic disease.

11127. The method for making a medical device of any one of items 10835-11085 wherein the neurostimulator is adapted for treating or preventing migraine.

11128. The method for making a medical device of any one of items 10835-11085 wherein the neurostimulator is adapted for treating or preventing sleep disorders.

11129. The method for making a medical device of any one of items 10835-11085 wherein the neurostimulator is adapted for treating or preventing dementia.

11130. The method for making a medical device of any one of items 10835-11085 wherein the neurostimulator is adapted for treating or preventing Alzheimer's disease.

11131. A method for making a medical device comprising: combining a cardiac rhythm management device (i.e., an electrical device) and an anti-scarring agent or a composition comprising an anti-scarring agent, wherein the agent inhibits scarring between the device and a host into which the device is implanted.

11132. The method of item 11131 wherein the agent is an adensosine A2A receptor antagonist.

11133. The method of item 11131 wherein the agent is an AKT inhibitor.

11134. The method of item 11131 wherein the agent is an alpha 2 integrin antagonist, wherein the alpha 2 integrin antagonist is Pharmaprojects No. 5754 (Merck KgaA).

11135. The method of item 11131 wherein the agent is an alpha 4 integrin antagonist.

11136. The method of item 11131 wherein the agent is an alpha 7 nicotinic receptor agonist.

11137. The method of item 11131 wherein the agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

11138. The method of item 11131 wherein the agent is an apoptosis antagonist.

11139. The method of item 11131 wherein the agent is an apoptosis activator.

11140. The method of item 11131 wherein the agent is a beta 1 integrin antagonist.

11141. The method of item 11131 wherein the agent is a beta tubulin inhibitor.

11142. The method of item 11131 wherein the agent is a blocker of enzyme production in Hepatitis C.

11143. The method of item 11131 wherein the agent is a Bruton's tyrosine kinase inhibitor.

11144. The method of item 11131 wherein the agent is a calcineurin inhibitor.

11145. The method of item 11131 wherein the agent is a caspase 3 inhibitor.

11146. The method of item 11131 wherein the agent is a CC chemokine receptor antagonist.

11147. The method of item 11131 wherein the agent is a cell cycle inhibitor selected from the group consisting of SNS-595 (Sunesis), synthadotin, KRX-0403, and an analogue or derivative thereof.

11148. The method of item 11131 wherein the agent is a cathepsin B inhibitor.

11149. The method of item 11131 wherein the agent is a cathepsin K inhibitor, wherein the cathepsin K inhibitor is 462795 (GlaxoSmithKline), INPL-022-D6 (Amura Therapeutics), or an analogue or derivative thereof.

11150. The method of item 11131 wherein the agent is a cathepsin L inhibitor.

11151. The method of item 11131 wherein the agent is a CD40 antagonist.

11152. The method of item 11131 wherein the agent is a chemokine receptor agonist.

11153. The method of item 11131 wherein the agent is a chymase inhibitor.

11154. The method of item 11131 wherein the agent is a collagenase antagonist.

11155. The method of item 11131 wherein the agent is a CXCR antagonist.

11156. The method of item 11131 wherein the agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

11157. The method of item 11131 wherein the agent is a cyclooxygenase 1 inhibitor.

11158. The method of item 11131 wherein the agent is a DHFR inhibitor.

11159. The method of item 11131 wherein the agent is a dual integrin inhibitor.

11160. The method of item 11131 wherein the agent is an elastase inhibitor.

11161. The method of item 11131 wherein the agent is an elongation factor-1 alpha inhibitor.

11162. The method of item 11131 wherein the agent is an endothelial growth factor antagonist.

11163. The method of item 11131 wherein the agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

11164. The method of item 11131 wherein the agent is an endotoxin antagonist

11165. The method of item 11131 wherein the agent is an epothilone and tubulin binder.

11166. The method of item 11131 wherein the agent is an estrogen receptor antagonist.

11167. The method of item 11131 wherein the agent is an FGF inhibitor.

11168. The method of item 11131 wherein the agent is a farnexyl transferase inhibitor.

11169. The method of item 11131 wherein the agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

11170. The method of item 11131 wherein the agent is an FLT-3 kinase inhibitor.

11171. The method of item 11131 wherein the agent is an FGF receptor kinase inhibitor

11172. The method of item 11131 wherein the agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

11173. The method of item 11131 wherein the agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

11174. The method of item 11131 wherein the agent is a histone deacetylase inhibitor.

11175. The method of item 11131 wherein the agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

11176. The method of item 11131 wherein the agent is an ICAM inhibitor.

11177. The method of item 11131 wherein the agent is an IL, ICE and IRAK antagonist, wherein the antagonist is a CJ-14877, CP-424174 (Pfizer), NF-61 (Negma-Lerads), and an analogue or derivative thereof.

11178. The method of item 11131 wherein the agent is an IL-2 inhibitor.

11179. The method of item 11131 wherein the agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

11180. The method of item 11131 wherein the agent is an IMPDH (inosine monophosphate).

11181. The method of item 11131 wherein the agent is an integrin antagonist.

11182. The method of item 11131 wherein the agent is an interleukin antagonist.

11183. The method of item 11131 wherein the agent is an inhibitor of type III receptor tyrosine kinase.

11184. The method of item 11131 wherein the agent is an irreversible inhibitor of enzyme methionine aminopeptidase type 2.

11185. The method of item 11131 wherein the agent is an isozyme selective delta protein kinase C inhibitor.

11186. The method of item 11131 wherein the agent a JAK3 enzyme inhibitor.

11187. The method of item 11131 wherein the agent is a JNK inhibitor.

11188. The method of item 11131 wherein the agent is a kinase inhibitor.

11189. The method of item 11131 wherein the agent is kinesin antagonist.

11190. The method of item 11131 wherein the agent is a kinesin antagonist.

11191. The method of item 11131 wherein the agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

11192. The method of item 11131 wherein the agent is an MAP kinase inhibitor.

11193. The method of item 11131 wherein the agent is a matrix metalloproteinase inhibitor.

11194. The method of item 11131 wherein the agent is an MCP-CCR2 inhibitor.

11195. The method of item 11131 wherein the agent is an mTOR inhibitor.

11196. The method of item 11131 wherein the agent is an mTOR kinase inhibitor.

11197. The method of item 11131 wherein the agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

11198. The method of item 11131 wherein the agent is an MIF inhibitor.

11199. The method of item 11131 wherein the agent is an MMP inhibitor.

11200. The method of item 11131 wherein the agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

11201. The method of item 11131 wherein the agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

11202. The method of item 11131 wherein the agent is a nitric oxide agonist.

11203. The method of item 11131 wherein the agent is an ornithine decarboxylase inhibitor.

11204. The method of item 11131 wherein the agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

11205. The method of item 11131 wherein the agent is a palmitoyl-protein thioesterase inhibitor.

11206. The method of item 11131 wherein the agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

11207. The method of item 11131 wherein the agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

11208. The method of item 11131 wherein the agent is a phosphatase inhibitor

11209. The method of item 11131 wherein the agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

11210. The method of item 11131 wherein the agent is a PKC inhibitor.

11211. The method of item 11131 wherein the agent is a platelet activating factor antagonist.

11212. The method of item 11131 wherein the agent is a platelet-derived growth factor receptor kinase inhibitor.

11213. The method of item 11131 wherein the agent is a prolyl hydroxylase inhibitor.

11214. The method of item 11131 wherein the agent is a polymorphonuclear neutrophil inhibitor.

11215. The method of item 11131 wherein the agent is a protein kinase B inhibitor.

11216. The method of item 11131 wherein the agent is a protein kinase C stimulant.

11217. The method of item 11131 wherein the agent is a purine nucleoside analogue.

11218. The method of item 11131 wherein the agent is a purinoreceptor P2X antagonist.

11219. The method of item 11131 wherein the agent is a Raf kinase inhibitor.

11220. The method of item 11131 wherein the agent is a reversible inhibitor of ErbB1 and ErbB2.

11221. The method of item 11131 wherein the agent is a ribonucleoside triphosphate reductase inhibitor.

11222. The method of item 11131 wherein the agent is an SDF-1 antagonist.

11223. The method of item 11131 wherein the agent is a sheddase inhibitor.

11224. The method of item 11131 wherein the agent is an SRC inhibitor.

11225. The method of item 11131 wherein the agent is a stromelysin inhibitor.

11226. The method of item 11131 wherein the agent is an Syk kinase inhibitor.

11227. The method of item 11131 wherein the agent is a telomerase inhibitor.

11228. The method of item 11131 wherein the agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

11229. The method of item 11131 wherein the agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), vT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

11230. The method of item 11131 wherein the agent is a Toll receptor inhibitor.

11231. The method of item 11131 wherein the agent is a tubulin antagonist.

11232. The method of item 11131 wherein the agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

11233. The method of item 11131 wherein the agent is a VEGF inhibitor.

11234. The method of item 11131 wherein the agent is a vitamin D receptor agonist.

11235. The method of item 11131 wherein the agent is ZD-6474 (an angiogenesis inhibitor).

11236. The method of item 11131 wherein the agent is AP-23573 (an mTOR inhibitor).

11237. The method of item 11131 wherein the agent is synthadotin (a tubulin antagonist).

11238. The method of item 11131 wherein the agent is S-0885 (a collagenase inhibitor).

11239. The method of item 11131 wherein the agent is aplidine (an elongation factor-1 alpha inhibitor).

11240. The method of item 11131 wherein the agent is ixabepilone (an epithilone).

11241. The method of item 11131 wherein the agent is IDN-5390 (an angiogenesis inhibitor).

11242. The method of item 11131 wherein the agent is SB-2723005 (an angiogenesis inhibitor).

11243. The method of item 11131 wherein the agent is ABT-518 (an angiogenesis inhibitor).

11244. The method of item 11131 wherein the agent is combretastatin (an angiogenesis inhibitor).

11245. The method of item 11131 wherein the agent is anecortave acetate (an angiogenesis inhibitor).

11246. The method of item 11131 wherein the agent is SB-715992 (a kinesin antagonist).

11247. The method of item 11131 wherein the agent is temsirolimus (an mTOR inhibitor).

11248. The method of item 11131 wherein the agent is adalimumab (a TNFα antagonist).

11249. The method of item 11131, wherein the composition comprises a polymer.

11250. The method of item 11131, wherein the composition comprises a polymeric carrier.

11251. The method of item 11131 wherein the anti-scarring agent inhibits adhesion between the medical device and a host into which the medical device is implanted.

11252. The method of item 11131 wherein the medical device delivers the anti-scarring agent locally to tissue proximate to the medical device.

11253. The method of item 11131 wherein the medical device has a coating that comprises the anti-scarring agent.

11254. The method of item 11131, wherein the medical device has a coating that comprises the agent and is disposed on a surface of the electrical device.

11255. The method of item 11131, wherein the medical device has a coating that comprises the agent and directly contacts the electrical device.

11256. The method of item 11131, wherein the medical device has a coating that comprises the agent and indirectly contacts the electrical device.

11257. The method of item 11131, wherein the medical device has a coating that comprises the agent and partially covers the electrical device.

11258. The method of item 11131, wherein the medical device has a coating that comprises the agent and completely covers the electrical device.

11259. The method of item 11131, wherein the medical device has a uniform coating.

11260. The method of item 11131, wherein the medical device has a non-uniform coating.

11261. The method of item 11131, wherein the medical device has a discontinuous coating.

11262. The method of item 11131, wherein the medical device has a patterned coating.

11263. The method of item 11131, wherein the medical device has a coating with a thickness of 100 μm or less.

11264. The method of item 11131, wherein the medical device has a coating with a thickness of 10 μm or less.

11265. The method of item 11131, wherein the medical device has a coating, and the coating adheres to the surface of the electrical device upon deployment of the electrical device.

11266. The method of item 11131, wherein the medical device has a coating, and wherein the coating is stable at room temperature for a period of 1 year.

11267. The method of item 11131, wherein the medical device has a coating, and wherein the anti-scarring agent is present in the coating in an amount ranging between about 0.0001% to about 1% by weight.

11268. The method of item 11131, wherein the medical device has a coating, and wherein the anti-scarring agent is present in the coating in an amount ranging between about 1% to about 10% by weight.

11269. The method of item 11131, wherein the medical device has a coating, and wherein the anti-scarring agent is present in the coating in an amount ranging between about 10% to about 25% by weight.

11270. The method of item 11131, wherein the medical device has a coating, and wherein the anti-scarring agent is present in the coating in an amount ranging between about 25% to about 70% by weight.

11271. The method of item 11131, wherein the medical device has a coating, and wherein the coating further comprises a polymer.

11272. The method of item 11131, wherein the medical device has a first coating having a first composition and a second coating having a second composition.

11273. The method of item 11131, wherein the medical device has a first coating having a first composition and a second coating having a second composition, wherein the first composition and the second composition are different.

11274. The method of item 11131, wherein the composition comprises a polymer.

11275. The method of item 11131, wherein the composition comprises a polymeric carrier.

11276. The method of item 11131, wherein the composition comprises a polymeric carrier, and wherein the polymeric carrier comprises a copolymer.

11277. The method of item 11131, wherein the composition comprises a polymeric carrier, and wherein the polymeric carrier comprises a block copolymer.

11278. The method of item 11131, wherein the composition comprises a polymeric carrier, and wherein the polymeric carrier comprises a random copolymer.

11279. The method of item 11131, wherein the composition comprises a polymeric carrier, and wherein the polymeric carrier comprises a biodegradable polymer.

11280. The method of item 11131, wherein the composition comprises a polymeric carrier, and wherein the polymeric carrier comprises a non-biodegradable polymer.

11281. The method of item 11131, wherein the composition comprises a polymeric carrier, and wherein the polymeric carrier comprises a hydrophilic polymer.

11282. The method of item 11131, wherein the composition comprises a polymeric carrier, and wherein the polymeric carrier comprises a hydrophobic polymer.

11283. The method of item 11131, wherein the composition comprises a polymeric carrier, and wherein the polymeric carrier comprises a polymer having hydrophilic domains.

11284. The method of item 11131, wherein the composition comprises a polymeric carrier, and wherein the polymeric carrier comprises a polymer having hydrophobic domains.

11285. The method of item 11131, wherein the composition comprises a polymeric carrier, and wherein the polymeric carrier comprises a non-conductive polymer.

11286. The method of item 11131, wherein the composition comprises a polymeric carrier, and wherein the polymeric carrier comprises an elastomer.

11287. The method of item 11131, wherein the composition comprises a polymeric carrier, and wherein the polymeric carrier comprises a hydrogel.

11288. The method of item 11131, wherein the composition comprises a polymeric carrier, and wherein the polymeric carrier comprises a silicone polymer.

11289. The method of item 11131, wherein the composition comprises a polymeric carrier, and wherein the polymeric carrier comprises a hydrocarbon polymer.

11290. The method of item 11131, wherein the composition comprises a polymeric carrier, and wherein the polymeric carrier comprises a styrene-derived polymer.

11291. The method of item 11131, wherein the composition comprises a polymeric carrier, and wherein the polymeric carrier comprises a butadiene polymer.

11292. The method of item 11131, wherein the composition comprises a polymeric carrier, and wherein the polymeric carrier comprises a macromer.

11293. The method of item 11131, wherein the composition comprises a polymeric carrier, and wherein the polymeric carrier comprises a poly(ethylene glycol) polymer.

11294. The method of item 11131 wherein the composition comprises a polymeric carrier, and wherein the polymeric carrier comprises an amorphous polymer.

11295. The method of item 11131, wherein the medical device comprises a lubricious coating.

11296. The method of item 11131 wherein the anti-scarring agent is located within pores or holes of the medical device.

11297. The method of item 11131 wherein the anti-scarring agent is located within a channel, lumen, or divet of the medical device.

11298. The method of item 11131, wherein the medical device further comprises a second pharmaceutically active agent.

11299. The method of item 11131 wherein the medical device further comprises an anti-inflammatory agent.

11300. The method of item 11131 wherein the medical device further comprises an agent that inhibits infection.

11301. The method of item 11131 wherein the medical device further comprises an agent that inhibits infection, and wherein the agent is an anthracycline.

11302. The method of item 11131 wherein the medical device further comprises an agent that inhibits infection, and wherein the agent is doxorubicin.

11303. The method of item 11131 wherein the medical device further comprises an agent that inhibits infection, and wherein the agent is mitoxantrone.

11304. The method of item 11131 wherein the medical device further comprises an agent that inhibits infection, and wherein the agent is a fluoropyrimidine.

11305. The method of item 11131 wherein the medical device further comprises an agent that inhibits infection, and wherein the agent is 5-fluorouracil (5-FU).

11306. The method of item 11131 wherein the medical device further comprises an agent that inhibits infection, and wherein the agent is a folic acid antagonist.

11307. The method of item 11131 wherein the medical device further comprises an agent that inhibits infection, and wherein the agent is methotrexate.

11308. The method of item 11131 wherein the medical device further comprises an agent that inhibits infection, and wherein the agent is a podophylotoxin.

11309. The method of item 11131 wherein the medical device further comprises an agent that inhibits infection, and wherein the agent is etoposide.

11310. The method of item 11131 wherein the medical device further comprises an agent that inhibits infection, and wherein the agent is a camptothecin.

11311. The method of item 11131 wherein the medical device further comprises an agent that inhibits infection, and wherein the agent is a hydroxyurea.

11312. The method of item 11131 wherein the medical device further comprises an agent that inhibits infection, and wherein the agent is a platinum complex.

11313. The method of item 11131 wherein the medical device further comprises an agent that inhibits infection, and wherein the agent is cisplatin.

11314. The method of item 11131 wherein the medical device further comprises an anti-thrombotic agent.

11315. The method of item 11131 wherein the medical device further comprises a visualization agent.

11316. The method of item 11131 wherein the medical device further comprises a visualization agent, wherein the visualization agent is a radiopaque material, and wherein the radiopaque material further comprises a metal, a halogenated compound, or a barium containing compound.

11317. The method of item 11131 wherein the medical device further comprises a visualization agent, wherein the visualization agent is a radiopaque material, and wherein the radiopaque material further comprises barium, tantalum, or technetium.

11318. The method of item 11131 wherein the medical device further comprises a visualization agent, and wherein the visualization agent is a MRI responsive material.

11319. The method of item 11131 wherein the medical device further comprises a visualization agent, and wherein the visualization agent further comprises a gadolinium chelate.

11320. The method of item 11131 wherein the medical device further comprises a visualization agent, and wherein the visualization agent further comprises iron, magnesium, manganese, copper, or chromium.

11321. The method of item 11131 wherein the medical device further comprises a visualization agent, and wherein the visualization agent further comprises an iron oxide compound.

11322. The method of item 11131 wherein the medical device further comprises a visualization agent, and wherein the visualization agent further comprises a dye, pigment, or colorant.

11323. The method of item 11131 wherein the medical device further comprises an echogenic material.

11324. The method of item 11131 wherein the medical device further comprises an echogenic material, and wherein the echogenic material is in the form of a coating.

11325. The method of item 11131 wherein the medical device is sterile.

11326. The method of item 11131 wherein the anti-scarring agent is released into tissue in the vicinity of the medical device after deployment of the medical device.

11327. The method of item 11131 wherein the anti-scarring agent is released into tissue in the vicinity of the medical device after deployment of the medical device, and wherein the tissue is connective tissue.

11328. The method of item 11131 wherein the anti-scarring agent is released into tissue in the vicinity of the medical device after deployment of the medical device, and wherein the tissue is muscle tissue.

11329. The method of item 11131 wherein the anti-scarring agent is released into tissue in the vicinity of the medical device after deployment of the medical device, and wherein the tissue is nerve tissue.

11330. The method of item 11131 wherein the anti-scarring agent is released into tissue in the vicinity of the medical device after deployment of the medical device, and wherein the tissue is epithelium tissue.

11331. The method of item 11131 wherein the anti-scarring agent is released in effective concentrations from the medical device over a period ranging from the time of deployment of the medical device to about 1 year.

11332. The method of item 11131 wherein the anti-scarring agent is released in effective concentrations from the medical device over a period ranging from about 1 month to 6 months.

11333. The method of item 11131 wherein the anti-scarring agent is released in effective concentrations from the medical device over a period ranging from about 1-90 days.

11334. The method of item 11131 wherein the anti-scarring agent is released in effective concentrations from the medical device at a constant rate.

11335. The method of item 11131 wherein the anti-scarring agent is released in effective concentrations from the medical device at an increasing rate.

11336. The method of item 11131 wherein the anti-scarring agent is released in effective concentrations from the medical device at a decreasing rate.

11337. The method of item 11131 wherein the anti-scarring agent is released in effective concentrations from the composition comprising the anti-scarring agent by diffusion over a period ranging from the time of deployment of the medical device to about 90 days.

11338. The method of item 11131 wherein the anti-scarring agent is released in effective concentrations from the composition comprising the anti-scarring agent by erosion of the composition over a period ranging from the time of deployment of the medical device to about 90 days.

11339. The method of item 11131 wherein the medical device comprises about 0.01 μg to about 10 μg of the anti-scarring agent.

11340. The method of item 11131 wherein the medical device comprises about 10 μg to about 10 mg of the anti-scarring agent.

11341. The method of item 11131 wherein the medical device comprises about 10 mg to about 250 mg of the anti-scarring agent.

11342. The method of item 11131 wherein the medical device comprises about 250 mg to about 1000 mg of the anti-scarring agent.

11343. The method of item 11131 wherein the medical device comprises about 1000 mg to about 2500 mg of the anti-scarring agent.

11344. The method of item 11131 wherein a surface of the medical device comprises less than 0.01 μg of the anti-scarring agent per mm² of medical device surface to which the anti-scarring agent is applied.

11345. The method of item 11131 wherein a surface of the medical device comprises about 0.01 μg to about 1 μg of the anti-scarring agent per mm² of medical device surface to which the anti-scarring agent is applied.

11346. The method of item 11131 wherein a surface of the medical device comprises about 1 μg to about 10 μg of the anti-scarring agent per mm² of medical device surface to which the anti-scarring agent is applied.

11347. The method of item 11131 wherein a surface of the medical device comprises about 10 μg to about 250 μg of the anti-scarring agent per mm² of medical device surface to which the anti-scarring agent is applied.

11348. The method of item 11131 wherein a surface of the medical device comprises about 250 μg to about 1000 μg of the anti-scarring agent of anti-scarring agent per mm² of medical device surface to which the anti-scarring agent is applied.

11349. The method of item 11131 wherein a surface of the medical device comprises about 1000 μg to about 2500 μg of the anti-scarring agent per mm² of medical device surface to which the anti-scarring agent is applied.

11350. The method of item 11131 wherein the combining is performed by direct affixing the agent or the composition to the electrical device.

11351. The method of item 11131 wherein the combining is performed by spraying the agent or the component onto the electrical device.

11352. The method of item 11131 wherein the combining is performed by electrospraying the agent or the composition onto the electrical device.

11353. The method of item 11131 wherein the combining is performed by dipping the electrical device into a solution comprising the agent or the composition.

11354. The method of item 11131 wherein the combining is performed by covalently attaching the agent or the composition to the electrical device.

11355. The method of item 11131 wherein the combining is performed by non-covalently attaching the agent or the composition to the electrical device.

11356. The method of item 11131 wherein the combining is performed by coating the electrical device with a substance that contains the agent or the composition.

11357. The method of item 11131 wherein the combining is performed by coating the electrical device with a substance that absorbs the agent.

11358. The method of item 11131 wherein the combining is performed by interweaving the electrical device with a thread composed of, or coated with, the agent or the composition.

11359. The method of item 11131 wherein the combining is performed by completely covering the electrical device with a sleeve that contains the agent or the composition.

11360. The method of item 11131 wherein the combining is performed by covering a portion of the electrical device with a sleeve that contains the agent or the composition.

11361. The method of item 11131 wherein the combining is performed by completely covering the electrical device with a cover that contains the agent or the composition.

11362. The method of item 11131 wherein the combining is performed by covering a portion of the electrical device with a cover that contains the agent or the composition.

11363. The method of item 11131 wherein the combining is performed by completely covering the electrical device with an electrospun fabric that contains the agent or the composition.

11364. The method of item 11131 wherein the combining is performed by covering a portion of the electrical device with an electrospun fabric that contains the agent or the composition.

11365. The method of item 11131 wherein the combining is performed by completely covering the electrical device with a mesh that contains the agent or the composition.

11366. The method of item 11131 wherein the combining is performed by covering a portion of the electrical device with a mesh that contains the agent or the composition.

11367. The method of item 11131 wherein the combining is performed by constructing a portion of the electrical device with the agent or the composition.

11368. The method of item 11131 wherein the combining is performed by impregnating the electrical device with the agent or the composition.

11369. The method of item 11131 wherein the combining is performed by constructing a portion of the electrical device from a degradable polymer that releases the agent.

11370. The method of item 11131 wherein the combining is performed by dipping the electrical device into a solution that comprise the agent and an inert solvent for the electrical device.

11371. The method of item 11131 wherein the combining is performed by dipping the electrical device into a solution that comprises the agent and a solvent that will swell the electrical device.

11372. The method of item 11131 wherein the combining is performed by dipping the electrical device into a solution that comprises the agent and a solvent that will dissolve the electrical device.

11373. The method of item 11131 wherein the combining is performed by dipping the electrical device into a solution that comprises the agent, a polymer and an inert solvent for the electrical device.

11374. The method of item 11131 wherein the combining is performed by dipping the electrical device into a solution that comprises the agent, a polymer and a solvent that will swell the electrical device.

11375. The method of item 11131 wherein the combining is performed by dipping the electrical device into a solution that comprises the agent, a polymer and a solvent that will dissolve the electrical device.

11376. The method of item 11131 wherein the combining is performed by spraying the electrical device into a solution that comprises the agent and an inert solvent for the electrical device.

11377. The method of item 11131 wherein the combining is performed by spraying the electrical device into a solution that comprises the agent and a solvent that will swell the electrical device.

11378. The method of item 11131 wherein the combining is performed by spraying the electrical device into a solution that comprises the agent and a solvent that will dissolve the electrical device.

11379. The method of item 11131 wherein the combining is performed by spraying the electrical device into a solution that comprises the agent, a polymer and an inert solvent for the electrical device.

11380. The method of item 11131 wherein the combining is performed by spraying the electrical device into a solution that comprises the agent, a polymer and a solvent that will swell the electrical device.

11381. The method of item 11131 wherein the combining is performed by spraying the electrical device into a solution that comprises the agent, a polymer and a solvent that will dissolve the electrical device.

11382. The method for making a medical device of any one of items 11131-11381 wherein the cardiac rhythm management device is an implantable pulse generator.

11383. The method for making a medical device of any one of items 11131-11381 wherein the cardiac rhythm management device is an electrical lead.

11384. The method for making a medical device of any one of items 11131-11381 wherein the cardiac rhythm management device is a stimulation lead.

11385. The method for making a medical device of any one of items 11131-11381 wherein the cardiac rhythm management device is a simulation catheter lead.

11386. The method for making a medical device of any one of items 11131-11381 wherein the cardiac rhythm management device is a microstimulator.

11387. The method for making a medical device of any one of items 11131-11381 wherein the cardiac rhythm management device is battery powered.

11388. The method for making a medical device of any one of items 11131-11381 wherein the cardiac rhythm management device is radio frequency powered.

11389. The method for making a medical device of any one of items 11131-11381 wherein the cardiac rhythm management device is both battery and radio frequency powered.

11390. The method for making a medical device of any one of items 11131-11381 wherein the cardiac rhythm management device is a cardiac pacemaker.

11391. The method for making a medical device of any one of items 11131-11381 wherein the cardiac rhythm management device is an implantable cardioverter defibrillator system.

11392. The method for making a medical device of any one of items 11131-11381 wherein the cardiac rhythm management device is a cardiac lead.

11393. The method for making a medical device of any one of items 11131-11381 wherein the cardiac rhythm management device is a pacer lead.

11394. The method for making a medical device of any one of items 11131-11381 wherein the cardiac rhythm management device is an endocardial lead.

11395. The method for making a medical device of any one of items 11131-11381 wherein the cardiac rhythm management device is a cardioversion/defibrillator lead.

11396. The method for making a medical device of any one of items 11131-11381 wherein the cardiac rhythm management device is an epicardial lead.

11397. The method for making a medical device of any one of items 11131-11381 wherein the cardiac rhythm management device is an epicardial defibrillator lead.

11398. The method for making a medical device of any one of items 11131-11381 wherein the cardiac rhythm management device is a patch defibrillator.

11399. The method for making a medical device of any one of items 11131-11381 wherein the cardiac rhythm management device is a patch defibrillator lead.

11400. The method for making a medical device of any one of items 11131-11381 wherein the cardiac rhythm management device is an electrical patch.

11401. The method for making a medical device of any one of items 11131-11381 wherein the cardiac rhythm management device is a transvenous lead.

11402. The method for making a medical device of any one of items 11131-11381 wherein the cardiac rhythm management device is an active fixation lead.

11403. The method for making a medical device of any one of items 11131-11381 wherein the cardiac rhythm management device is a passive fixation lead.

11404. The method for making a medical device of any one of items 11131-11381 wherein the cardiac rhythm management device is a sensing lead.

11405. The method for making a medical device of any one of items 11131-11381 wherein the cardiac rhythm management device is a defibrillator.

11406. The method for making a medical device of any one of items 11131-11381 wherein the cardiac rhythm management device is an implantable sensor.

11407. The method for making a medical device of any one of items 11131-11381 wherein the cardiac rhythm management device is a left ventricular assist device.

11408. The method for making a medical device of any one of items 11131-11381 wherein the cardiac rhythm management device is a pulse generator.

11409. The method for making a medical device of any one of items 11131-11381 wherein the cardiac rhythm management device is a patch lead.

11410. The method for making a medical device of any one of items 11131-11381 wherein the cardiac rhythm management device is an electrical patch.

11411. The method for making a medical device of any one of items 11131-11381 wherein the cardiac rhythm management device is a cardiac stimulator.

11412. The method for making a medical device of any one of items 11131-11381 wherein the cardiac rhythm management device is an electrical deviceable sensor.

11413. The method for making a medical device of any one of items 11131-11381 wherein the cardiac rhythm management device is an electrical deviceable pump.

11414. The method for making a medical device of any one of items 11131-11381 wherein the cardiac rhythm management device is a dural patch.

11415. The method for making a medical device of any one of items 11131-11381 wherein the cardiac rhythm management device is a ventricular peritoneal shunt.

11416. The method for making a medical device of any one of items 11131-11381 wherein the cardiac rhythm management device is a ventricular atrial shunt.

11417. The method for making a medical device of any one of items 11131-11381 wherein the cardiac rhythm management device is adapted for treating or preventing epidural fibrosis post-laminectomy.

11418. The method for making a medical device of any one of items 11131-11381 wherein the cardiac rhythm management device is adapted for treating or preventing cardiac rhythm abnormalities.

11419. The method for making a medical device of any one of items 11131-11381 wherein the cardiac rhythm management device is adapted for treating or preventing atrial rhythm abnormalities.

11420. The method for making a medical device of any one of items 11131-11381 wherein the cardiac rhythm management device is adapted for treating or preventing conduction abnormalities.

11421. The method for making a medical device of any one of items 11131-11381 wherein the cardiac rhythm management device is adapted for treating or preventing ventricular rhythm abnormalities.

11422. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host.

11423. The method of item 11422 comprising: (a) infiltrating a tissue of a host where the electrical device is to be, or has been, implanted with an anti-fibrotic agent, and (b) implanting the electrical device into the host.

11424. The method of item 11422 comprising: (a) infiltrating a tissue of a host where the electrical device is to be, or has been, implanted with an anti-infective agent, and (b) implanting the electrical device into the host.

11425. The method of item 11422 comprising: (a) infiltrating a tissue of a host where the electrical device is to be, or has been, implanted with a polymer; and (b) implanting the electrical device into the host.

11426. The method of item 11422 comprising: (a) infiltrating a tissue of a host where the electrical device is to be, or has been, implanted with a composition comprising an anti-fibrotic agent and a polymer, and (b) implanting the electrical device into the host.

11427. The method of item 11422 comprising: (a) infiltrating a tissue of a host where the electrical device is to be, or has been, implanted with a composition comprising an anti-infective agent and a polymer, and (b) implanting the electrical device into the host.

11428. The method of item 11422 comprising: (a) infiltrating a tissue of a host where the electrical device is to be, or has been, implanted with a composition comprising an anti-fibrotic agent, an anti-infective agent and a polymer, and (b) implanting the electrical device into the host.

11429. The method of item 11422 wherein the agent is an adensosine A2A receptor antagonist.

11430. The method of item 11422 wherein the anti-fibrotic agent is an AKT inhibitor.

11431. The method of item 11422 wherein the anti-fibrotic agent is an alpha 2 integrin antagonist, wherein the alpha 2 integrin antagonist is Pharmaprojects No. 5754 (Merck KgaA).

11432. The method of item 11422 wherein the anti-fibrotic agent is an alpha 4 integrin antagonist.

11433. The method of item 11422 wherein the anti-fibrotic agent is an alpha 7 nicotinic receptor agonist.

11434. The method of item 11422 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

11435. The method of item 11422 wherein the anti-fibrotic agent is an apoptosis antagonist.

11436. The method of item 11422 wherein the anti-fibrotic agent is an apoptosis activator.

11437. The method of item 11422 wherein the anti-fibrotic agent is a beta 1 integrin antagonist.

11438. The method of item 11422 wherein the anti-fibrotic agent is a beta tubulin inhibitor.

11439. The method of item 11422 wherein the anti-fibrotic agent is a blocker of enzyme production in Hepatitis C.

11440. The method of item 11422 wherein the anti-fibrotic agent is a Bruton's tyrosine kinase inhibitor.

11441. The method of item 11422 wherein the anti-fibrotic agent is a calcineurin inhibitor.

11442. The method of item 11422 wherein the anti-fibrotic agent is a caspase 3 inhibitor.

11443. The method of item 11422 wherein the anti-fibrotic agent is a CC chemokine receptor antagonist.

11444. The method of item 11422 wherein the anti-fibrotic agent is a cell cycle inhibitor selected from the group consisting of SNS-595 (Sunesis), synthadotin, KRX-0403, and an analogue or derivative thereof.

11445. The method of item 11422 wherein the anti-fibrotic agent is a cathepsin B inhibitor.

11446. The method of item 11422 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

11447. The method of item 11422 wherein the anti-fibrotic agent is a cathepsin L inhibitor.

11448. The method of item 11422 wherein the anti-fibrotic agent is a CD40 antagonist.

11449. The method of item 11422 wherein the anti-fibrotic agent is a chemokine receptor agonist.

11450. The method of item 11422 wherein the anti-fibrotic agent is a chymase inhibitor.

11451. The method of item 11422 wherein the anti-fibrotic agent is a collagenase antagonist.

11452. The method of item 11422 wherein the anti-fibrotic agent is a CXCR antagonist.

11453. The method of item 11422 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

11454. The method of item 11422 wherein the anti-fibrotic agent is a cyclooxygenase 1 inhibitor.

11455. The method of item 11422 wherein the anti-fibrotic agent is a DHFR inhibitor.

11456. The method of item 11422 wherein the anti-fibrotic agent is a dual integrin inhibitor.

11457. The method of item 11422 wherein the anti-fibrotic agent is an elastase inhibitor.

11458. The method of item 11422 wherein the anti-fibrotic agent is an elongation factor-1 alpha inhibitor.

11459. The method of item 11422 wherein the anti-fibrotic agent is an endothelial growth factor antagonist.

11460. The method of item 11422 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

11461. The method of item 11422 wherein the anti-fibrotic agent is an endotoxin antagonist.

11462. The method of item 11422 wherein the anti-fibrotic agent is an epothilone and tubulin binder.

11463. The method of item 11422 wherein the anti-fibrotic agent is an estrogen receptor antagonist.

11464. The method of item 11422 wherein the anti-fibrotic agent is an FGF inhibitor.

11465. The method of item 11422 wherein the anti-fibrotic agent is a farnexyl transferase inhibitor.

11466. The method of item 11422 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

11467. The method of item 11422 wherein the anti-fibrotic agent is an FLT-3 kinase inhibitor.

11468. The method of item 11422 wherein the anti-fibrotic agent is an FGF receptor kinase inhibitor.

11469. The method of item 11422 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

11470. The method of item 11422 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

11471. The method of item 11422 wherein the anti-fibrotic agent is a histone deacetylase inhibitor.

11472. The method of item 11422 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

11473. The method of item 11422 wherein the anti-fibrotic agent is an ICAM inhibitor.

11474. The method of item 11422 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

11475. The method of item 11422 wherein the anti-fibrotic agent is an IL-2 inhibitor.

11476. The method of item 11422 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 11878841-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

11477. The method of item 11422 wherein the anti-fibrotic agent is an IMPDH (inosine monophosphate).

11478. The method of item 11422 wherein the anti-fibrotic agent is an integrin antagonist.

11479. The method of item 11422 wherein the anti-fibrotic agent is an interleukin antagonist.

11480. The method of item 11422 wherein the anti-fibrotic agent is an inhibitor of type III receptor tyrosine kinase.

11481. The method of item 11422 wherein the anti-fibrotic agent is an irreversible inhibitor of enzyme methionine aminopeptidase type 2.

11482. The method of item 11422 wherein the anti-fibrotic agent is an isozyme selective delta protein kinase C inhibitor.

11483. The method of item 11422 wherein the anti-fibrotic agent a JAK3 enzyme inhibitor.

11484. The method of item 11422 wherein the anti-fibrotic agent is a JNK inhibitor.

11485. The method of item 11422 wherein the anti-fibrotic agent is a kinase inhibitor.

11486. The method of item 11422 wherein the anti-fibrotic agent is kinesin antagonist.

11487. The method of item 11422 wherein the anti-fibrotic agent is a kinesin antagonist.

11488. The method of item 11422 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

11489. The method of item 11422 wherein the anti-fibrotic agent is an MAP kinase inhibitor.

11490. The method of item 11422 wherein the anti-fibrotic agent is a matrix metalloproteinase inhibitor.

11491. The method of item 11422 wherein the anti-fibrotic agent is an MCP-CCR2 inhibitor.

11492. The method of item 11422 wherein the anti-fibrotic agent is an mTOR inhibitor.

11493. The method of item 11422 wherein the anti-fibrotic agent is an mTOR kinase inhibitor.

11494. The method of item 11422 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DMI (Genentech), vinorelbine, and an analogue or derivative thereof.

11495. The method of item 11422 wherein the anti-fibrotic agent is an MIF inhibitor.

11496. The method of item 11422 wherein the anti-fibrotic agent is an MMP inhibitor.

11497. The method of item 11422 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

11498. The method of item 11422 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

11499. The method of item 11422 wherein the anti-fibrotic agent is a nitric oxide agonist.

11500. The method of item 11422 wherein the anti-fibrotic agent is an ornithine decarboxylase inhibitor.

11501. The method of item 11422 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

11502. The method of item 11422 wherein the anti-fibrotic agent is a palmitoyl-protein thioesterase inhibitor.

11503. The method of item 11422 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

11504. The method of item 11422 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

11505. The method of item 11422 wherein the anti-fibrotic agent is a phosphatase inhibitor.

11506. The method of item 11422 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

11507. The method of item 11422 wherein the anti-fibrotic agent is a PKC inhibitor.

11508. The method of item 11422 wherein the anti-fibrotic agent is a platelet activating factor antagonist.

11509. The method of item 11422 wherein the anti-fibrotic agent is a platelet-derived growth factor receptor kinase inhibitor.

11510. The method of item 11422 wherein the anti-fibrotic agent is a prolyl hydroxylase inhibitor.

11511. The method of item 11422 wherein the anti-fibrotic agent is a polymorphonuclear neutrophil inhibitor.

11512. The method of item 11422 wherein the anti-fibrotic agent is a protein kinase B inhibitor.

11513. The method of item 11422 wherein the anti-fibrotic agent is a protein kinase C stimulant.

11514. The method of item 11422 wherein the anti-fibrotic agent is a purine nucleoside analogue.

11515. The method of item 11422 wherein the anti-fibrotic agent is a purinoreceptor P2X antagonist.

11516. The method of item 11422 wherein the anti-fibrotic agent is a Raf kinase inhibitor.

11517. The method of item 11422 wherein the anti-fibrotic agent is a reversible inhibitor of ErbB1 and ErbB2.

11518. The method of item 11422 wherein the anti-fibrotic agent is a ribonucleoside triphosphate reductase inhibitor.

11519. The method of item 11422 wherein the anti-fibrotic agent is an SDF-1 antagonist.

11520. The method of item 11422 wherein the anti-fibrotic agent is a sheddase inhibitor.

11521. The method of item 11422 wherein the anti-fibrotic agent is an SRC inhibitor.

11522. The method of item 11422 wherein the anti-fibrotic agent is a stromelysin inhibitor.

11523. The method of item 11422 wherein the anti-fibrotic agent is an Syk kinase inhibitor.

11524. The method of item 11422 wherein the anti-fibrotic agent is a telomerase inhibitor.

11525. The method of item 11422 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

11526. The method of item 11422 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

11527. The method of item 11422 wherein the anti-fibrotic agent is a Toll receptor inhibitor.

11528. The method of item 11422 wherein the anti-fibrotic agent is a tubulin antagonist.

11529. The method of item 11422 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-1 3 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

11530. The method of item 11422 wherein the anti-fibrotic agent is a VEGF inhibitor.

11531. The method of item 11422 wherein the anti-fibrotic agent is a vitamin D receptor agonist.

11532. The method of item 11422 wherein the anti-fibrotic agent is ZD-6474 (an angiogenesis inhibitor).

11533. The method of item 11422 wherein the anti-fibrotic agent is AP-23573 (an mTOR inhibitor).

11534. The method of item 11422 wherein the anti-fibrotic agent is synthadotin (a tubulin antagonist).

11535. The method of item 11422 wherein the anti-fibrotic agent is S-0885 (a collagenase inhibitor).

11536. The method of item 11422 wherein the anti-fibrotic agent is aplidine (an elongation factor-1 alpha inhibitor).

11537. The method of item 11422 wherein the anti-fibrotic agent is ixabepilone (an epithilone).

11538. The method of item 11422 wherein the anti-fibrotic agent is IDN-5390 (an angiogenesis inhibitor).

11539. The method of item 11422 wherein the anti-fibrotic agent is SB-2723005 (an angiogenesis inhibitor).

11540. The method of item 11422 wherein the anti-fibrotic agent is ABT-518 (an angiogenesis inhibitor).

11541. The method of item 11422 wherein the anti-fibrotic agent is combretastatin (an angiogenesis inhibitor).

11542. The method of item 11422 wherein the anti-fibrotic agent is anecortave acetate (an angiogenesis inhibitor).

11543. The method of item 11422 wherein the anti-fibrotic agent is SB-715992 (a kinesin antagonist).

11544. The method of item 11422 wherein the anti-fibrotic agent is temsirolimus (an mTOR inhibitor).

11545. The method of item 11422 wherein the anti-fibrotic agent is adalimumab (a TNFα antagonist).

11546. The method of item 11422 wherein the anti-infective agent is an anthracycline.

11547. The method of item 11422 wherein the anti-infective agent is doxorubicin.

11548. The method of item 11422 wherein the anti-infective agent is mitoxantrone.

11549. The method of item 11422 wherein the anti-infective agent is a fluoropyrimidine.

11550. The method of item 11422 wherein the anti-infective agent is 5-fluorouracil (5-FU).

11551. The method of item 11422 wherein the anti-infective agent is a folic acid antagonist.

11552. The method of item 11422 wherein the anti-infective agent is methotrexate.

11553. The method of item 11422 wherein the anti-infective agent is a podophylotoxin.

11554. The method of item 11422 wherein the anti-infective agent is etoposide.

11555. The method of item 11422 wherein the anti-infective agent is camptothecin.

11556. The method of item 11422 wherein the anti-infective agent is a hydroxyurea.

11557. The method of item 11422 wherein the anti-infective agent is a platinum complex.

11558. The method of item 11422 wherein the anti-infective agent is cisplatin.

11559. The method of item 11422 wherein the composition comprises an anti-thrombotic agent.

11560. The method of item 11422 wherein the polymer is formed from reactants comprising a naturally occurring polymer.

11561. The method of item 11422 wherein the polymer is formed from reactants comprising protein.

11562. The method of item 11422 wherein the polymer is formed from reactants comprising carbohydrate.

11563. The method of item 11422 wherein the polymer is formed from reactants comprising biodegradable polymer.

11564. The method of item 11422 wherein the polymer is formed from reactants comprising nonbiodegradable polymer.

11565. The method of item 11422 wherein the polymer is formed from reactants comprising collagen.

11566. The method of item 11422 wherein the polymer is formed from reactants comprising methylated collagen.

11567. The method of item 11422 wherein the polymer is formed from reactants comprising fibrinogen.

11568. The method of item 11422 wherein the polymer is formed from reactants comprising thrombin.

11569. The method of item 11422 wherein the polymer is formed from reactants comprising blood plasma.

11570. The method of item 11422 wherein the polymer is formed from reactants comprising calcium salt.

11571. The method of item 11422 wherein the polymer is formed from reactants comprising an antifibronolytic agent.

11572. The method of item 11422 wherein the polymer is formed from reactants comprising fibrinogen analog.

11573. The method of item 11422 wherein the polymer is formed from reactants comprising albumin.

11574. The method of item 11422 wherein the polymer is formed from reactants comprising plasminogen.

11575. The method of item 11422 wherein the polymer is formed from reactants comprising von Willebrands factor.

11576. The method of item 11422 wherein the polymer is formed from reactants comprising Factor VIII.

11577. The method of item 11422 wherein the polymer is formed from reactants comprising hypoallergenic collagen.

11578. The method of item 11422 wherein the polymer is formed from reactants comprising atelopeptidic collagen.

11579. The method of item 11422 wherein the polymer is formed from reactants comprising telopeptide collagen.

11580. The method of item 11422 wherein the polymer is formed from reactants comprising crosslinked collagen.

11581. The method of item 11422 wherein the polymer is formed from reactants comprising aprotinin.

11582. The method of item 11422 wherein the polymer is formed from reactants comprising epsilon-amino-n-caproic acid.

11583. The method of item 11422 wherein the polymer is formed from reactants comprising gelatin.

11584. The method of item 11422 wherein the polymer is formed from reactants comprising protein conjugates.

11585. The method of item 11422 wherein the polymer is formed from reactants comprising gelatin conjugates.

11586. The method of item 11422 wherein the polymer is formed from reactants comprising a synthetic polymer.

11587. The method of item 11422 wherein the polymer is formed from reactants comprising a synthetic isocyanate-containing compound.

11588. The method of item 11422 wherein the polymer is formed from reactants comprising a synthetic thiol-containing compound.

11589. The method of item 11422 wherein the polymer is formed from reactants comprising a synthetic compound containing at least two thiol groups.

11590. The method of item 11422 wherein the polymer is formed from reactants comprising a synthetic compound containing at least three thiol groups.

11591. The method of item 11422 wherein the polymer is formed from reactants comprising a synthetic compound containing at least four thiol groups.

11592. The method of item 11422 wherein the polymer is formed from reactants comprising a synthetic amino-containing compound.

11593. The method of item 11422 wherein the polymer is formed from reactants comprising a synthetic compound containing at least two amino groups.

11594. The method of item 11422 wherein the polymer is formed from reactants comprising a synthetic compound containing at least three amino groups.

11595. The method of item 11422 wherein the polymer is formed from reactants comprising a synthetic compound containing at least four amino groups.

11596. The method of item 11422 wherein the polymer is formed from reactants comprising a synthetic compound comprising a carbonyl-oxygen-succinimidyl group.

11597. The method of item 11422 wherein the polymer is formed from reactants comprising a synthetic compound comprising at least two carbonyl-oxygen-succinimidyl groups.

11598. The method of item 11422 wherein the polymer is formed from reactants comprising a synthetic compound comprising at least three carbonyl-oxygen-succinimidyl groups.

11599. The method of item 11422 wherein the polymer is formed from reactants comprising a synthetic compound comprising at least four carbonyl-oxygen-succinimidyl groups.

11600. The method of item 11422 wherein the polymer is formed from reactants comprising a synthetic polyalkylene oxide-containing compound.

11601. The method of item 11422 wherein the polymer is formed from reactants comprising a synthetic compound comprising both polyalkylene oxide and biodegradable polyester blocks.

11602. The method of item 11422 wherein the polymer is formed from reactants comprising a synthetic polyalkylene oxide-containing compound having reactive amino groups.

11603. The method of item 11422 wherein the polymer is formed from reactants comprising a synthetic polyalkylene oxide-containing compound having reactive thiol groups.

11604. The method of item 11422 wherein the polymer is formed from reactants comprising a synthetic polyalkylene oxide-containing compound having reactive carbonyl-oxygen-succinimidyl groups.

11605. The method of item 11422 wherein the polymer is formed from reactants comprising a synthetic compound comprising a biodegradable polyester block.

11606. The method of item 11422 wherein the polymer is formed from reactants comprising a synthetic polymer formed in whole or part from lactic acid or lactide.

11607. The method of item 11422 wherein the polymer is formed from reactants comprising a synthetic polymer formed in whole or part from glycolic acid or glycolide.

11608. The method of item 11422 wherein the polymer is formed from reactants comprising polylysine.

11609. The method of item 11422 wherein the polymer is formed from reactants comprising (a) protein and (b) a compound comprising a polyalkylene oxide portion.

11610. The method of item 11422 wherein the polymer is formed from reactants comprising (a) protein and (b) polylysine.

11611. The method of item 11422 wherein the polymer is formed from reactants comprising (a) protein and (b) a compound having at least four thiol groups.

11612. The method of item 11422 wherein the polymer is formed from reactants comprising (a) protein and (b) a compound having at least four amino groups.

11613. The method of item 11422 wherein the polymer is formed from reactants comprising (a) protein and (b) a compound having at least four carbonyl-oxygen-succinimide groups.

11614. The method of item 11422 wherein the polymer is formed from reactants comprising (a) protein and (b) a compound having a biodegradable region formed from reactants selected from lactic acid, lactide, glycolic acid, glycolide, and epison-caprolactone.

11615. The method of item 11422 wherein the polymer is formed from reactants comprising (a) collagen and (b) a compound comprising a polyalkylene oxide portion.

11616. The method of item 11422 wherein the polymer is formed from reactants comprising (a) collagen and (b) polylysine.

11617. The method of item 11422 wherein the polymer is formed from reactants comprising (a) collagen and (b) a compound having at least four thiol groups.

11618. The method of item 11422 wherein the polymer is formed from reactants comprising (a) collagen and (b) a compound having at least four amino groups.

11619. The method of item 11422 wherein the polymer is formed from reactants comprising (a) collagen and (b) a compound having at least four carbonyl-oxygen-succinimide groups.

11620. The method of item 11422 wherein the polymer is formed from reactants comprising (a) collagen and (b) a compound having a biodegradable region formed from reactants selected from lactic acid, lactide, glycolic acid, glycolide, and epison-caprolactone.

11621. The method of item 11422 wherein the polymer is formed from reactants comprising (a) methylated collagen and (b) a compound comprising a polyalkylene oxide portion.

11622. The method of item 11422 wherein the polymer is formed from reactants comprising (a) methylated collagen and (b) polylysine.

11623. The method of item 11422 wherein the polymer is formed from reactants comprising (a) methylated collagen and (b) a compound having at least four thiol groups.

11624. The method of item 11422 wherein the polymer is formed from reactants comprising (a) methylated collagen and (b) a compound having at least four amino groups.

11625. The method of item 11422 wherein the polymer is formed from reactants comprising (a) methylated collagen and (b) a compound having at least four carbonyl-oxygen-succinimide groups.

11626. The method of item 11422 wherein the polymer is formed from reactants comprising (a) methylated collagen and (b) a compound having a biodegradable region formed from reactants selected from lactic acid, lactide, glycolic acid, glycolide, and epison-caprolactone.

11627. The method of item 11422 wherein the polymer is formed from reactants comprising hyaluronic acid.

11628. The method of item 11422 wherein the polymer is formed from reactants comprising a hyaluronic acid derivative.

11629. The method of item 11422 wherein the polymer is formed from reactants comprising pentaerythritol poly(ethylene glycol)ether tetra-sulfhydryl of number average molecular weight between 3,000 and 30,000.

11630. The method of item 11422 wherein the polymer is formed from reactants comprising pentaerythritol poly(ethylene glycol)ether tetra-amino of number average molecular weight between 3,000 and 30,000.

11631. The method of item 11422 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

11632. The method of item 11422 wherein the composition comprises a colorant.

11633. The method of item 11422 wherein the composition is sterile.

11634. The method for implanting an electrical device of any one of items 11422-11633 wherein the electrical device is a neurostimulator.

11635. The method for implanting an electrical device of any one of items 11422-11633 wherein the electrical device is a spinal cord stimulator.

11636. The method for implanting an electrical device of any one of items 11422-11633 wherein the electrical device is a brain stimulator.

11637. The method for implanting an electrical device of any one of items 11422-11633 wherein the electrical device is a vagus nerve stimulator.

11638. The method for implanting an electrical device of any one of items 11422-11633 wherein the electrical device is a sacral nerve stimulator.

11639. The method for implanting an electrical device of any one of items 11422-11633 wherein the electrical device is a gastric nerve stimulator.

11640. The method for implanting an electrical device of any one of items 11422-11633 wherein the electrical device is an auditory nerve stimulator.

11641. The method for implanting an electrical device of any one of items 11422-11633 wherein the electrical device delivers stimulation to organs.

11642. The method for implanting an electrical device of any one of items 11422-11633 wherein the electrical device delivers stimulation to bone.

11643. The method for implanting an electrical device of any one of items 11422-11633 wherein the electrical device delivers stimulation to muscles.

11644. The method for implanting an electrical device of any one of items 11422-11633 wherein the electrical device delivers stimulation to tissues.

11645. The method for implanting an electrical device of any one of items 11422-11633 wherein the electrical device is a device for continuous subarachnoid infusion.

11646. The method for implanting an electrical device of any one of items 11422-11633 wherein the electrical device is an implantable electrode.

11647. The method for implanting an electrical device of any one of items 11422-11633 wherein the electrical device is an implantable pulse generator.

11648. The method for implanting an electrical device of any one of items 11422-11633 wherein the electrical device is an electrical lead.

11649. The method for implanting an electrical device of any one of items 11422-11633 wherein the electrical device is a stimulation lead.

11650. The method for implanting an electrical device of any one of items 11422-11633 wherein the electrical device is a simulation catheter lead.

11651. The method for implanting an electrical device of any one of items 11422-11633 wherein the electrical device is cochlear implant.

11652. The method for implanting an electrical device of any one of items 11422-11633 wherein the electrical device is a microstimulator.

11653. The method for implanting an electrical device of any one of items 11422-11633 wherein the electrical device is battery powered.

11654. The method for implanting an electrical device of any one of items 11422-11633 wherein the electrical device is radio frequency powered.

11655. The method for implanting an electrical device of any one of items 11422-11633 wherein the electrical device is both battery and radio frequency powered.

11656. The method for implanting an electrical device of any one of items 11422-11633 wherein the electrical device is a cardiac rhythm management device.

11657. The method for implanting an electrical device of any one of items 11422-11633 wherein the electrical device is a cardiac pacemaker.

11658. The method for implanting an electrical device of any one of items 11422-11633 wherein the electrical device is an implantable cardioverter defibrillator system.

11659. The method for implanting an electrical device of any one of items 11422-11633 wherein the electrical device is a cardiac lead.

11660. The method for implanting an electrical device of any one of items 11422-11633 wherein the electrical device is a pacer lead.

11661. The method for implanting an electrical device of any one of items 11422-11633 wherein the electrical device is an endocardial lead.

11662. The method for implanting an electrical device of any one of items 11422-11633 wherein the electrical device is a cardioversion/defibrillator lead.

11663. The method for implanting an electrical device of any one of items 11422-11633 wherein the electrical device is an epicardial lead.

11664. The method for implanting an electrical device of any one of items 11422-11633 wherein the electrical device is an epicardial defibrillator lead.

11665. The method for implanting an electrical device of any one of items 11422-11633 wherein the electrical device is a patch defibrillator.

11666. The method for implanting an electrical device of any one of items 11422-11633 wherein the electrical device is a patch defibrillator lead.

11667. The method for implanting an electrical device of any one of items 11422-11633 wherein the electrical device is an electrical patch.

11668. The method for implanting an electrical device of any one of items 11422-11633 wherein the electrical device is a transvenous lead.

11669. The method for implanting an electrical device of any one of items 11422-11633 wherein the electrical device is an active fixation lead.

11670. The method for implanting an electrical device of any one of items 11422-11633 wherein the electrical device is a passive fixation lead.

11671. The method for implanting an electrical device of any one of items 11422-11633 wherein the electrical device is a sensing lead.

11672. The method for implanting an electrical device of any one of items 11422-11633 wherein the electrical device is a defibrillator.

11673. The method for implanting an electrical device of any one of items 11422-11633 wherein the electrical device is an implantable sensor.

11674. The method for implanting an electrical device of any one of items 11422-11633 wherein the electrical device is a left ventricular assist device.

11675. The method for implanting an electrical device of any one of items 11422-11633 wherein the electrical device is a pulse generator.

11676. The method for implanting an electrical device of any one of items 11422-11633 wherein the electrical device is a patch lead.

11677. The method for implanting an electrical device of any one of items 11422-11633 wherein the electrical device is an electrical patch.

11678. The method for implanting an electrical device of any one of items 11422-11633 wherein the electrical device is a cardiac stimulator.

11679. The method for implanting an electrical device of any one of items 11422-11633 wherein the electrical device is an electrical deviceable sensor.

11680. The method for implanting an electrical device of any one of items 11422-11633 wherein the electrical device is an electrical deviceable pump.

11681. The method for implanting an electrical device of any one of items 11422-11633 wherein the electrical device is a dural patch.

11682. The method for implanting an electrical device of any one of items 11422-11633 wherein the electrical device is a ventricular peritoneal shunt.

11683. The method for implanting an electrical device of any one of items 11422-11633 wherein the electrical device is a ventricular atrial shunt.

11684. The method for implanting an electrical device of any one of items 11422-11633 wherein the electrical device is adapted for treating or preventing epidural fibrosis post-laminectomy.

11685. The method for implanting an electrical device of any one of items 11422-11633 wherein the electrical device is adapted for treating or preventing cardiac rhythm abnormalities.

11686. The method for implanting an electrical device of any one of items 11422-11633 wherein the electrical device is adapted for treating or preventing atrial rhythm abnormalities.

11687. The method for implanting an electrical device of any one of items 11422-11633 wherein the electrical device is adapted for treating or preventing conduction abnormalities.

11688. The method for implanting an electrical device of any one of items 11422-11633 wherein the electrical device is adapted for treating or preventing ventricular rhythm abnormalities.

11689. The method for implanting an electrical device of any one of items 11422-11633 wherein the electrical device is adapted for treating or preventing pain.

11690. The method for implanting an electrical device of any one of items 11422-11633 wherein the electrical device is adapted for treating or preventing epilepsy.

11691. The method for implanting an electrical device of any one of items 11422-11633 wherein the electrical device is adapted for treating or preventing Parkinson's disease.

11692. The method for implanting an electrical device of any one of items 11422-11633 wherein the electrical device is adapted for treating or preventing movement disorders.

11693. The method for implanting an electrical device of any one of items 11422-11633 wherein the electrical device is adapted for treating or preventing obesity.

11694. The method for implanting an electrical device of any one of items 11422-11633 wherein the electrical device is adapted for treating or preventing depression.

11695. The method for implanting an electrical device of any one of items 11422-11633 wherein the electrical device is adapted for treating or preventing anxiety.

11696. The method for implanting an electrical device of any one of items 11422-11633 wherein the electrical device is adapted for treating or preventing hearing loss.

11697. The method for implanting an electrical device of any one of items 11422-11633 wherein the electrical device is adapted for treating or preventing ulcers.

11698. The method for implanting an electrical device of any one of items 11422-11633 wherein the electrical device is adapted for treating or preventing deep vein thrombosis.

11699. The method for implanting an electrical device of any one of items 11422-11633 wherein the electrical device is adapted for treating or preventing muscular atrophy.

11700. The method for implanting an electrical device of any one of items 11422-11633 wherein the electrical device is adapted for treating or preventing joint stiffness.

11701. The method for implanting an electrical device of any one of items 11422-11633 wherein the electrical device is adapted for treating or preventing muscle spasms.

11702. The method for implanting an electrical device of any one of items 11422-11633 wherein the electrical device is adapted for treating or preventing osteoporosis.

11703. The method for implanting an electrical device of any one of items 11422-11633 wherein the electrical device is adapted for treating or preventing scoliosis.

11704. The method for implanting an electrical device of any one of items 11422-11633 wherein the electrical device is adapted for treating or preventing spinal disc degeneration.

11705. The method for implanting an electrical device of any one of items 11422-11633 wherein the electrical device is adapted for treating or preventing spinal cord injury.

11706. The method for implanting an electrical device of any one of items 11422-11633 wherein the electrical device is adapted for treating or preventing urinary dysfunction.

11707. The method for implanting an electrical device of any one of items 11422-11633 wherein the electrical device is adapted for treating or preventing gastroparesis.

11708. The method for implanting an electrical device of any one of items 11422-11633 wherein the electrical device is adapted for treating or preventing malignancy.

11709. The method for implanting an electrical device of any one of items 11422-11633 wherein the electrical device is adapted for treating or preventing arachnoiditis.

11710. The method for implanting an electrical device of any one of items 11422-11633 wherein the electrical device is adapted for treating or preventing chronic disease.

11711. The method for implanting an electrical device of any one of items 11422-11633 wherein the electrical device is adapted for treating or preventing migraine.

11712. The method for implanting an electrical device of any one of items 11422-11633 wherein the electrical device is adapted for treating or preventing sleep disorders.

11713. The method for implanting an electrical device of any one of items 11422-11633 wherein the electrical device is adapted for treating or preventing dementia.

11714. The method for implanting an electrical device of any one of items 11422-11633 wherein the electrical device is adapted for treating or preventing Alzheimer's disease.

11715. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a neurostimulator for treating chronic pain.

11716. The method of item 11715 comprising: (a) infiltrating a tissue of a host where the electrical device is to be, or has been, implanted with an anti-fibrotic agent, and (b) implanting the electrical device into the host.

11717. The method of item 11715 comprising: (a) infiltrating a tissue of a host where the electrical device is to be, or has been, implanted with an anti-infective agent, and (b) implanting the electrical device into the host.

11718. The method of item 11715 comprising: (a) infiltrating a tissue of a host where the electrical device is to be, or has been, implanted with a polymer; and (b) implanting the electrical device into the host.

11719. The method of item 11715 comprising: (a) infiltrating a tissue of a host where the electrical device is to be, or has been, implanted with a composition comprising an anti-fibrotic agent and a polymer, and (b) implanting the electrical device into the host.

11720. The method of item 11715 comprising: (a) infiltrating a tissue of a host where the electrical device is to be, or has been, implanted with a composition comprising an anti-infective agent and a polymer, and (b) implanting the electrical device into the host.

11721. The method of item 11715 comprising: (a) infiltrating a tissue of a host where the electrical device is to be, or has been, implanted with a composition comprising an anti-fibrotic agent, an anti-infective agent and a polymer, and (b) implanting the electrical device into the host.

11722. The method of item 11715 wherein the agent is an adensosine A2A receptor antagonist.

11723. The method of item 11715 wherein the anti-fibrotic agent is an AKT inhibitor.

11724. The method of item 11715 wherein the anti-fibrotic agent is an alpha 2 integrin antagonist, wherein the alpha 2 integrin antagonist is Pharmaprojects No. 5754 (Merck KgaA).

11725. The method of item 11715 wherein the anti-fibrotic agent is an alpha 4 integrin antagonist.

11726. The method of item 11715 wherein the anti-fibrotic agent is an alpha 7 nicotinic receptor agonist.

11727. The method of item 11715 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

11728. The method of item 11715 wherein the anti-fibrotic agent is an apoptosis antagonist.

11729. The method of item 11715 wherein the anti-fibrotic agent is an apoptosis activator.

11730. The method of item 11715 wherein the anti-fibrotic agent is a beta 1 integrin antagonist.

11731. The method of item 11715 wherein the anti-fibrotic agent is a beta tubulin inhibitor.

11732. The method of item 11715 wherein the anti-fibrotic agent is a blocker of enzyme production in Hepatitis C.

11733. The method of item 11715 wherein the anti-fibrotic agent is a Bruton's tyrosine kinase inhibitor.

11734. The method of item 11715 wherein the anti-fibrotic agent is a calcineurin inhibitor.

11735. The method of item 11715 wherein the anti-fibrotic agent is a caspase 3 inhibitor.

11736. The method of item 11715 wherein the anti-fibrotic agent is a CC chemokine receptor antagonist.

11737. The method of item 11715 wherein the anti-fibrotic agent is a cell cycle inhibitor selected from the group consisting of SNS-595 (Sunesis), synthadotin, KRX-0403, and an analogue or derivative thereof.

11738. The method of item 11715 wherein the anti-fibrotic agent is a cathepsin B inhibitor.

11739. The method of item 11715 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

11740. The method of item 11715 wherein the anti-fibrotic agent is a cathepsin L inhibitor.

11741. The method of item 11715 wherein the anti-fibrotic agent is a CD40 antagonist.

11742. The method of item 11715 wherein the anti-fibrotic agent is a chemokine receptor agonist.

11743. The method of item 11715 wherein the anti-fibrotic agent is a chymase inhibitor.

11744. The method of item 11715 wherein the anti-fibrotic agent is a collagenase antagonist.

11745. The method of item 11715 wherein the anti-fibrotic agent is a CXCR antagonist.

11746. The method of item 11715 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

11747. The method of item 11715 wherein the anti-fibrotic agent is a cyclooxygenase 1 inhibitor.

11748. The method of item 11715 wherein the anti-fibrotic agent is a DHFR inhibitor.

11749. The method of item 11715 wherein the anti-fibrotic agent is a dual integrin inhibitor.

11750. The method of item 11715 wherein the anti-fibrotic agent is an elastase inhibitor.

11751. The method of item 11715 wherein the anti-fibrotic agent is an elongation factor-1 alpha inhibitor.

11752. The method of item 11715 wherein the anti-fibrotic agent is an endothelial growth factor antagonist.

11753. The method of item 11715 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

11754. The method of item 11715 wherein the anti-fibrotic agent is an endotoxin antagonist.

11755. The method of item 11715 wherein the anti-fibrotic agent is an epothilone and tubulin binder.

11756. The method of item 11715 wherein the anti-fibrotic agent is an estrogen receptor antagonist.

11757. The method of item 11715 wherein the anti-fibrotic agent is an FGF inhibitor.

11758. The method of item 11715 wherein the anti-fibrotic agent is a farnexyl transferase inhibitor.

11759. The method of item 11715 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

11760. The method of item 11715 wherein the anti-fibrotic agent is an FLT-3 kinase inhibitor.

11761. The method of item 11715 wherein the anti-fibrotic agent is an FGF receptor kinase inhibitor.

11762. The method of item 11715 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

11763. The method of item 11715 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

11764. The method of item 11715 wherein the anti-fibrotic agent is a histone deacetylase inhibitor.

11765. The method of item 11715 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

11766. The method of item 11715 wherein the anti-fibrotic agent is an ICAM inhibitor.

11767. The method of item 11715 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

11768. The method of item 11715 wherein the anti-fibrotic agent is an IL-2 inhibitor.

11769. The method of item 11715 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

11770. The method of item 11715 wherein the anti-fibrotic agent is an IMPDH (inosine monophosphate).

11771. The method of item 11715 wherein the anti-fibrotic agent is an integrin antagonist.

11772. The method of item 11715 wherein the anti-fibrotic agent is an interleukin antagonist.

11773. The method of item 11715 wherein the anti-fibrotic agent is an inhibitor of type III receptor tyrosine kinase.

11774. The method of item 11715 wherein the anti-fibrotic agent is an irreversible inhibitor of enzyme methionine aminopeptidase type 2.

11775. The method of item 11715 wherein the anti-fibrotic agent is an isozyme selective delta protein kinase C inhibitor.

11776. The method of item 11715 wherein the anti-fibrotic agent a JAK3 enzyme inhibitor.

11777. The method of item 11715 wherein the anti-fibrotic agent is a JNK inhibitor.

11778. The method of item 11715 wherein the anti-fibrotic agent is a kinase inhibitor.

11779. The method of item 11715 wherein the anti-fibrotic agent is kinesin antagonist.

11780. The method of item 11715 wherein the anti-fibrotic agent is a kinesin antagonist.

11781. The method of item 11715 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

11782. The method of item 11715 wherein the anti-fibrotic agent is an MAP kinase inhibitor.

11783. The method of item 11715 wherein the anti-fibrotic agent is a matrix metalloproteinase inhibitor.

11784. The method of item 11715 wherein the anti-fibrotic agent is an MCP-CCR2 inhibitor.

11785. The method of item 11715 wherein the anti-fibrotic agent is an mTOR inhibitor.

11786. The method of item 11715 wherein the anti-fibrotic agent is an mTOR kinase inhibitor.

11787. The method of item 11715 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

11788. The method of item 11715 wherein the anti-fibrotic agent is an MIF inhibitor.

11789. The method of item 11715 wherein the anti-fibrotic agent is an MMP inhibitor.

11790. The method of item 11715 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-1 05212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

11791. The method of item 11715 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

11792. The method of item 11715 wherein the anti-fibrotic agent is a nitric oxide agonist.

11793. The method of item 11715 wherein the anti-fibrotic agent is an ornithine decarboxylase inhibitor.

11794. The method of item 11715 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

11795. The method of item 11715 wherein the anti-fibrotic agent is a palmitoyl-protein thioesterase inhibitor.

11796. The method of item 11715 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

11797. The method of item 11715 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

11798. The method of item 11715 wherein the anti-fibrotic agent is a phosphatase inhibitor.

11799. The method of item 11715 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-1 53 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

11800. The method of item 11715 wherein the anti-fibrotic agent is a PKC inhibitor.

11801. The method of item 11715 wherein the anti-fibrotic agent is a platelet activating factor antagonist.

11802. The method of item 11715 wherein the anti-fibrotic agent is a platelet-derived growth factor receptor kinase inhibitor.

11803. The method of item 11715 wherein the anti-fibrotic agent is a prolyl hydroxylase inhibitor.

11804. The method of item 11715 wherein the anti-fibrotic agent is a polymorphonuclear neutrophil inhibitor.

11805. The method of item 11715 wherein the anti-fibrotic agent is a protein kinase B inhibitor.

11806. The method of item 11715 wherein the anti-fibrotic agent is a protein kinase C stimulant.

11807. The method of item 11715 wherein the anti-fibrotic agent is a purine nucleoside analogue.

11808. The method of item 11715 wherein the anti-fibrotic agent is a purinoreceptor P2X antagonist.

11809. The method of item 11715 wherein the anti-fibrotic agent is a Raf kinase inhibitor.

11810. The method of item 11715 wherein the anti-fibrotic agent is a reversible inhibitor of ErbB1 and ErbB2.

11811. The method of item 11715 wherein the anti-fibrotic agent is a ribonucleoside triphosphate reductase inhibitor.

11812. The method of item 11715 wherein the anti-fibrotic agent is an SDF-1 antagonist.

11813. The method of item 11715 wherein the anti-fibrotic agent is a sheddase inhibitor.

11814. The method of item 11715 wherein the anti-fibrotic agent is an SRC inhibitor.

11815. The method of item 11715 wherein the anti-fibrotic agent is a stromelysin inhibitor.

11816. The method of item 11715 wherein the anti-fibrotic agent is an Syk kinase inhibitor.

11817. The method of item 11715 wherein the anti-fibrotic agent is a telomerase inhibitor.

11818. The method of item 11715 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

11819. The method of item 11715 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

11820. The method of item 11715 wherein the anti-fibrotic agent is a Toll receptor inhibitor.

11821. The method of item 11715 wherein the anti-fibrotic agent is a tubulin antagonist.

11822. The method of item 11715 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

11823. The method of item 11715 wherein the anti-fibrotic agent is a VEGF inhibitor.

11824. The method of item 11715 wherein the anti-fibrotic agent is a vitamin D receptor agonist.

11825. The method of item 11715 wherein the anti-fibrotic agent is ZD-6474 (an angiogenesis inhibitor).

11826. The method of item 11715 wherein the anti-fibrotic agent is AP-23573 (an mTOR inhibitor).

11827. The method of item 11715 wherein the anti-fibrotic agent is synthadotin (a tubulin antagonist).

11828. The method of item 11715 wherein the anti-fibrotic agent is S-0885 (a collagenase inhibitor).

11829. The method of item 11715 wherein the anti-fibrotic agent is aplidine (an elongation factor-1 alpha inhibitor).

11830. The method of item 11715 wherein the anti-fibrotic agent is ixabepilone (an epithilone).

11831. The method of item 11715 wherein the anti-fibrotic agent is IDN-5390 (an angiogenesis inhibitor).

11832. The method of item 11715 wherein the anti-fibrotic agent is SB-2723005 (an angiogenesis inhibitor).

11833. The method of item 11715 wherein the anti-fibrotic agent is ABT-518 (an angiogenesis inhibitor).

11834. The method of item 11715 wherein the anti-fibrotic agent is combretastatin (an angiogenesis inhibitor).

11835. The method of item 11715 wherein the anti-fibrotic agent is anecortave acetate (an angiogenesis inhibitor).

11836. The method of item 11715 wherein the anti-fibrotic agent is SB-715992 (a kinesin antagonist).

11837. The method of item 11715 wherein the anti-fibrotic agent is temsirolimus (an mTOR inhibitor).

11838. The method of item 11715 wherein the anti-fibrotic agent is adalimumab (a TNFα antagonist).

11839. The method of item 11715 wherein the anti-infective agent is an anthracycline.

11840. The method of item 11715 wherein the anti-infective agent is doxorubicin.

11841. The method of item 11715 wherein the anti-infective agent is mitoxantrone.

11842. The method of item 11715 wherein the anti-infective agent is a fluoropyrimidine.

11843. The method of item 11715 wherein the anti-infective agent is 5-fluorouracil (5-FU).

11844. The method of item 11715 wherein the anti-infective agent is a folic acid antagonist.

11845. The method of item 11715 wherein the anti-infective agent is methotrexate.

11846. The method of item 11715 wherein the anti-infective agent is a podophylotoxin.

11847. The method of item 11715 wherein the anti-infective agent is etoposide.

11848. The method of item 11715 wherein the anti-infective agent is camptothecin.

11849. The method of item 11715 wherein the anti-infective agent is a hydroxyurea.

11850. The method of item 11715 wherein the anti-infective agent is a platinum complex.

11851. The method of item 11715 wherein the anti-infective agent is cisplatin.

11852. The method of item 11715 wherein the composition comprises an anti-thrombotic agent.

11853. The method of item 11715 wherein the polymer is formed from reactants comprising a naturally occurring polymer.

11854. The method of item 11715 wherein the polymer is formed from reactants comprising protein.

11855. The method of item 11715 wherein the polymer is formed from reactants comprising carbohydrate.

11856. The method of item 11715 wherein the polymer is formed from reactants comprising biodegradable polymer.

11857. The method of item 11715 wherein the polymer is formed from reactants comprising nonbiodegradable polymer.

11858. The method of item 11715 wherein the polymer is formed from reactants comprising collagen.

11859. The method of item 11715 wherein the polymer is formed from reactants comprising methylated collagen.

11860. The method of item 11715 wherein the polymer is formed from reactants comprising fibrinogen.

11861. The method of item 11715 wherein the polymer is formed from reactants comprising thrombin.

11862. The method of item 11715 wherein the polymer is formed from reactants comprising blood plasma.

11863. The method of item 11715 wherein the polymer is formed from reactants comprising calcium salt.

11864. The method of item 11715 wherein the polymer is formed from reactants comprising an antifibronolytic agent.

11865. The method of item 11715 wherein the polymer is formed from reactants comprising fibrinogen analog.

11866. The method of item 11715 wherein the polymer is formed from reactants comprising albumin.

11867. The method of item 11715 wherein the polymer is formed from reactants comprising plasminogen.

11868. The method of item 11715 wherein the polymer is formed from reactants comprising von Willebrands factor.

11869. The method of item 11715 wherein the polymer is formed from reactants comprising Factor VIII.

11870. The method of item 11715 wherein the polymer is formed from reactants comprising hypoallergenic collagen.

11871. The method of item 11715 wherein the polymer is formed from reactants comprising atelopeptidic collagen.

11872. The method of item 11715 wherein the polymer is formed from reactants comprising telopeptide collagen.

11873. The method of item 11715 wherein the polymer is formed from reactants comprising crosslinked collagen.

11874. The method of item 11715 wherein the polymer is formed from reactants comprising aprotinin.

11875. The method of item 11715 wherein the polymer is formed from reactants comprising epsilon-amino-n-caproic acid.

11876. The method of item 11715 wherein the polymer is formed from reactants comprising gelatin.

11877. The method of item 11715 wherein the polymer is formed from reactants comprising protein conjugates.

11878. The method of item 11715 wherein the polymer is formed from reactants comprising gelatin conjugates.

11879. The method of item 11715 wherein the polymer is formed from reactants comprising a synthetic polymer.

11880. The method of item 11715 wherein the polymer is formed from reactants comprising a synthetic isocyanate-containing compound.

11881. The method of item 11715 wherein the polymer is formed from reactants comprising a synthetic thiol-containing compound.

11882. The method of item 11715 wherein the polymer is formed from reactants comprising a synthetic compound containing at least two thiol groups.

11883. The method of item 11715 wherein the polymer is formed from reactants comprising a synthetic compound containing at least three thiol groups.

11884. The method of item 11715 wherein the polymer is formed from reactants comprising a synthetic compound containing at least four thiol groups.

11885. The method of item 11715 wherein the polymer is formed from reactants comprising a synthetic amino-containing compound.

11886. The method of item 11715 wherein the polymer is formed from reactants comprising a synthetic compound containing at least two amino groups.

11887. The method of item 11715 wherein the polymer is formed from reactants comprising a synthetic compound containing at least three amino groups.

11888. The method of item 11715 wherein the polymer is formed from reactants comprising a synthetic compound containing at least four amino groups.

11889. The method of item 11715 wherein the polymer is formed from reactants comprising a synthetic compound comprising a carbonyl-oxygen-succinimidyl group.

11890. The method of item 11715 wherein the polymer is formed from reactants comprising a synthetic compound comprising at least two carbonyl-oxygen-succinimidyl groups.

11891. The method of item 11715 wherein the polymer is formed from reactants comprising a synthetic compound comprising at least three carbonyl-oxygen-succinimidyl groups.

11892. The method of item 11715 wherein the polymer is formed from reactants comprising a synthetic compound comprising at least four carbonyl-oxygen-succinimidyl groups.

11893. The method of item 11715 wherein the polymer is formed from reactants comprising a synthetic polyalkylene oxide-containing compound.

11894. The method of item 11715 wherein the polymer is formed from reactants comprising a synthetic compound comprising both polyalkylene oxide and biodegradable polyester blocks.

11895. The method of item 11715 wherein the polymer is formed from reactants comprising a synthetic polyalkylene oxide-containing compound having reactive amino groups.

11896. The method of item 11715 wherein the polymer is formed from reactants comprising a synthetic polyalkylene oxide-containing compound having reactive thiol groups.

11897. The method of item 11715 wherein the polymer is formed from reactants comprising a synthetic polyalkylene oxide-containing compound having reactive carbonyl-oxygen-succinimidyl groups.

11898. The method of item 11715 wherein the polymer is formed from reactants comprising a synthetic compound comprising a biodegradable polyester block.

11899. The method of item 11715 wherein the polymer is formed from reactants comprising a synthetic polymer formed in whole or part from lactic acid or lactide.

11900. The method of item 11715 wherein the polymer is formed from reactants comprising a synthetic polymer formed in whole or part from glycolic acid or glycolide.

11901. The method of item 11715 wherein the polymer is formed from reactants comprising polylysine.

11902. The method of item 11715 wherein the polymer is formed from reactants comprising (a) protein and (b) a compound comprising a polyalkylene oxide portion.

11903. The method of item 11715 wherein the polymer is formed from reactants comprising (a) protein and (b) polylysine.

11904. The method of item 11715 wherein the polymer is formed from reactants comprising (a) protein and (b) a compound having at least four thiol groups.

11905. The method of item 11715 wherein the polymer is formed from reactants comprising (a) protein and (b) a compound having at least four amino groups.

11906. The method of item 11715 wherein the polymer is formed from reactants comprising (a) protein and (b) a compound having at least four carbonyl-oxygen-succinimide groups.

11907. The method of item 11715 wherein the polymer is formed from reactants comprising (a) protein and (b) a compound having a biodegradable region formed from reactants selected from lactic acid, lactide, glycolic acid, glycolide, and epison-caprolactone.

11908. The method of item 11715 wherein the polymer is formed from reactants comprising (a) collagen and (b) a compound comprising a polyalkylene oxide portion.

11909. The method of item 11715 wherein the polymer is formed from reactants comprising (a) collagen and (b) polylysine.

11910. The method of item 11715 wherein the polymer is formed from reactants comprising (a) collagen and (b) a compound having at least four thiol groups.

11911. The method of item 11715 wherein the polymer is formed from reactants comprising (a) collagen and (b) a compound having at least four amino groups.

11912. The method of item 11715 wherein the polymer is formed from reactants comprising (a) collagen and (b) a compound having at least four carbonyl-oxygen-succinimide groups.

11913. The method of item 11715 wherein the polymer is formed from reactants comprising (a) collagen and (b) a compound having a biodegradable region formed from reactants selected from lactic acid, lactide, glycolic acid, glycolide, and epison-caprolactone.

11914. The method of item 11715 wherein the polymer is formed from reactants comprising (a) methylated collagen and (b) a compound comprising a polyalkylene oxide portion.

11915. The method of item 11715 wherein the polymer is formed from reactants comprising (a) methylated collagen and (b) polylysine.

11916. The method of item 11715 wherein the polymer is formed from reactants comprising (a) methylated collagen and (b) a compound having at least four thiol groups.

11917. The method of item 11715 wherein the polymer is formed from reactants comprising (a) methylated collagen and (b) a compound having at least four amino groups.

11918. The method of item 11715 wherein the polymer is formed from reactants comprising (a) methylated collagen and (b) a compound having at least four carbonyl-oxygen-succinimide groups.

11919. The method of item 11715 wherein the polymer is formed from reactants comprising (a) methylated collagen and (b) a compound having a biodegradable region formed from reactants selected from lactic acid, lactide, glycolic acid, glycolide, and epison-caprolactone.

11920. The method of item 11715 wherein the polymer is formed from reactants comprising hyaluronic acid.

11921. The method of item 11715 wherein the polymer is formed from reactants comprising a hyaluronic acid derivative.

11922. The method of item 11715 wherein the polymer is formed from reactants comprising pentaerythritol poly(ethylene glycol)ether tetra-sulfhydryl of number average molecular weight between 3,000 and 30,000.

11923. The method of item 11715 wherein the polymer is formed from reactants comprising pentaerythritol poly(ethylene glycol)ether tetra-amino of number average molecular weight between 3,000 and 30,000.

11924. The method of item 11715 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

11925. The method of item 11715 wherein the composition comprises a colorant.

11926. The method of item 11715 wherein the composition is sterile.

11927. The method for implanting an electrical device of items 11715-11926 wherein the chronic pain results from injury.

11928. The method for implanting an electrical device of items 11715-11926 wherein the chronic pain results from an illness.

11929. The method for implanting an electrical device of items 11715-11926 wherein the chronic pain results from scoliosis.

11930. The method for implanting an electrical device of items 11715-11926 wherein the chronic pain results from spinal disc degeneration.

11931. The method for implanting an electrical device of items 11715-11926 wherein the chronic pain results from malignancy.

11932. The method for implanting an electrical device of items 11715-11926 wherein the chronic pain results from arachnoiditis.

11933. The method for implanting an electrical device of items 11715-11926 wherein the chronic pain results from a chronic disease.

11934. The method for implanting an electrical device of items 11715-11926 wherein the chronic pain results from a pain syndrome.

11935. The method for implanting an electrical device of items 11715-11926 wherein the neurostimulator comprises a lead that delivers electrical stimulation to a nerve and an electrical connection that connects a power source to the lead.

11936. The method for implanting an electrical device of items 11715-11926 wherein the neurostimulator is adapted for spinal cord stimulation, and comprises a sensor that detects the position of the spine and a stimulator that emits pulses that decrease in amplitude when the back is in a supine position.

11937. The method for implanting an electrical device of items 11715-11926 wherein the neurostimulator comprises an electrode and a control circuit that generates pulses and rest period based on intervals corresponding to the host body's activity and regeneration period.

11938. The method for implanting an electrical device of items 11715-11926 wherein the neurostimulator comprises a stimulation catheter lead and an electrode.

11939. The method for implanting an electrical device of items 11715-11926 wherein the neurostimulator is a self-centering epidural spinal cord lead.

11940. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a neurostimulator for treating Parkinson's Disease.

11941. The method of item 11940 comprising: (a) infiltrating a tissue of a host where the electrical device is to be, or has been, implanted with an anti-fibrotic agent, and (b) implanting the electrical device into the host.

11942. The method of item 11940 comprising: (a) infiltrating a tissue of a host where the electrical device is to be, or has been, implanted with an anti-infective agent, and (b) implanting the electrical device into the host.

11943. The method of item 11940 comprising: (a) infiltrating a tissue of a host where the electrical device is to be, or has been, implanted with a polymer; and (b) implanting the electrical device into the host.

11944. The method of item 11940 comprising: (a) infiltrating a tissue of a host where the electrical device is to be, or has been, implanted with a composition comprising an anti-fibrotic agent and a polymer, and (b) implanting the electrical device into the host.

11945. The method of item 11940 comprising: (a) infiltrating a tissue of a host where the electrical device is to be, or has been, implanted with a composition comprising an anti-infective agent and a polymer, and (b) implanting the electrical device into the host.

11946. The method of item 11940 comprising: (a) infiltrating a tissue of a host where the electrical device is to be, or has been, implanted with a composition comprising an anti-fibrotic agent, an anti-infective agent and a polymer, and (b) implanting the electrical device into the host.

11947. The method of item 11940 wherein the agent is an adensosine A2A receptor antagonist.

11948. The method of item 11940 wherein the anti-fibrotic agent is an AKT inhibitor.

11949. The method of item 11940 wherein the anti-fibrotic agent is an alpha 2 integrin antagonist, wherein the alpha 2 integrin antagonist is Pharmaprojects No. 5754 (Merck KgaA).

11950. The method of item 11940 wherein the anti-fibrotic agent is an alpha 4 integrin antagonist.

11951. The method of item 11940 wherein the anti-fibrotic agent is an alpha 7 nicotinic receptor agonist.

11952. The method of item 11940 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

11953. The method of item 11940 wherein the anti-fibrotic agent is an apoptosis antagonist.

11954. The method of item 11940 wherein the anti-fibrotic agent is an apoptosis activator.

11955. The method of item 11940 wherein the anti-fibrotic agent is a beta 1 integrin antagonist.

11956. The method of item 11940 wherein the anti-fibrotic agent is a beta tubulin inhibitor.

11957. The method of item 11940 wherein the anti-fibrotic agent is a blocker of enzyme production in Hepatitis C.

11958. The method of item 11940 wherein the anti-fibrotic agent is a Bruton's tyrosine kinase inhibitor.

11959. The method of item 11940 wherein the anti-fibrotic agent is a calcineurin inhibitor.

11960. The method of item 11940 wherein the anti-fibrotic agent is a caspase 3 inhibitor.

11961. The method of item 11940 wherein the anti-fibrotic agent is a CC chemokine receptor antagonist.

11962. The method of item 11940 wherein the anti-fibrotic agent is a cell cycle inhibitor selected from the group consisting of SNS-595 (Sunesis), synthadotin, KRX-0403, and an analogue or derivative thereof.

11963. The method of item 11940 wherein the anti-fibrotic agent is a cathepsin B inhibitor.

11964. The method of item 11940 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

11965. The method of item 11940 wherein the anti-fibrotic agent is a cathepsin L inhibitor.

11966. The method of item 11940 wherein the anti-fibrotic agent is a CD40 antagonist.

11967. The method of item 11940 wherein the anti-fibrotic agent is a chemokine receptor agonist.

11968. The method of item 11940 wherein the anti-fibrotic agent is a chymase inhibitor.

11969. The method of item 11940 wherein the anti-fibrotic agent is a collagenase antagonist.

11970. The method of item 11940 wherein the anti-fibrotic agent is a CXCR antagonist.

11971. The method of item 11940 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

11972. The method of item 11940 wherein the anti-fibrotic agent is a cyclooxygenase I inhibitor.

11973. The method of item 11940 wherein the anti-fibrotic agent is a DHFR inhibitor.

11974. The method of item 11940 wherein the anti-fibrotic agent is a dual integrin inhibitor.

11975. The method of item 11940 wherein the anti-fibrotic agent is an elastase inhibitor.

11976. The method of item 11940 wherein the anti-fibrotic agent is an elongation factor-1 alpha inhibitor.

11977. The method of item 11940 wherein the anti-fibrotic agent is an endothelial growth factor antagonist.

11978. The method of item 11940 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

11979. The method of item 11940 wherein the anti-fibrotic agent is an endotoxin antagonist.

11980. The method of item 11940 wherein the anti-fibrotic agent is an epothilone and tubulin binder.

11981. The method of item 11940 wherein the anti-fibrotic agent is an estrogen receptor antagonist.

11982. The method of item 11940 wherein the anti-fibrotic agent is an FGF inhibitor.

11983. The method of item 11940 wherein the anti-fibrotic agent is a farnexyl transferase inhibitor.

11984. The method of item 11940 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

11985. The method of item 11940 wherein the anti-fibrotic agent is an FLT-3 kinase inhibitor.

11986. The method of item 11940 wherein the anti-fibrotic agent is an FGF receptor kinase inhibitor.

11987. The method of item 11940 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

11988. The method of item 11940 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

11989. The method of item 11940 wherein the anti-fibrotic agent is a histone deacetylase inhibitor.

11990. The method of item 11940 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

11991. The method of item 11940 wherein the anti-fibrotic agent is an ICAM inhibitor.

11992. The method of item 11940 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

11993. The method of item 11940 wherein the anti-fibrotic agent is an IL-2 inhibitor.

11994. The method of item 11940 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

11995. The method of item 11940 wherein the anti-fibrotic agent is an IMPDH (inosine monophosphate).

11996. The method of item 11940 wherein the anti-fibrotic agent is an integrin antagonist.

11997. The method of item 11940 wherein the anti-fibrotic agent is an interleukin antagonist.

11998. The method of item 11940 wherein the anti-fibrotic agent is an inhibitor of type III receptor tyrosine kinase.

11999. The method of item 11940 wherein the anti-fibrotic agent is an irreversible inhibitor of enzyme methionine aminopeptidase type 2.

12000. The method of item 11940 wherein the anti-fibrotic agent is an isozyme selective delta protein kinase C inhibitor.

12001. The method of item 11940 wherein the anti-fibrotic agent a JAK3 enzyme inhibitor.

12002. The method of item 11940 wherein the anti-fibrotic agent is a JNK inhibitor.

12003. The method of item 11940 wherein the anti-fibrotic agent is a kinase inhibitor.

12004. The method of item 11940 wherein the anti-fibrotic agent is kinesin antagonist.

12005. The method of item 11940 wherein the anti-fibrotic agent is a kinesin antagonist.

12006. The method of item 11940 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

12007. The method of item 11940 wherein the anti-fibrotic agent is an MAP kinase inhibitor.

12008. The method of item 11940 wherein the anti-fibrotic agent is a matrix metalloproteinase inhibitor.

12009. The method of item 11940 wherein the anti-fibrotic agent is an MCP-CCR2 inhibitor.

12010. The method of item 11940 wherein the anti-fibrotic agent is an mTOR inhibitor.

12011. The method of item 11940 wherein the anti-fibrotic agent is an mTOR kinase inhibitor.

12012. The method of item 11940 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

12013. The method of item 11940 wherein the anti-fibrotic agent is an MIF inhibitor.

12014. The method of item 11940 wherein the anti-fibrotic agent is an MMP inhibitor.

12015. The method of item 11940 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

12016. The method of item 11940 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

12017. The method of item 11940 wherein the anti-fibrotic agent is a nitric oxide agonist.

12018. The method of item 11940 wherein the anti-fibrotic agent is an ornithine decarboxylase inhibitor.

12019. The method of item 11940 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

12020. The method of item 11940 wherein the anti-fibrotic agent is a palmitoyl-protein thioesterase inhibitor.

12021. The method of item 11940 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

12022. The method of item 11940 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), k-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from CareX, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

12023. The method of item 11940 wherein the anti-fibrotic agent is a phosphatase inhibitor.

12024. The method of item 11940 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Go), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

12025. The method of item 11940 wherein the anti-fibrotic agent is a PKC inhibitor.

12026. The method of item 11940 wherein the anti-fibrotic agent is a platelet activating factor antagonist.

12027. The method of item 11940 wherein the anti-fibrotic agent is a platelet-derived growth factor receptor kinase inhibitor.

12028. The method of item 11940 wherein the anti-fibrotic agent is a prolyl hydroxylase inhibitor.

12029. The method of item 11940 wherein the anti-fibrotic agent is a polymorphonuclear neutrophil inhibitor.

12030. The method of item 11940 wherein the anti-fibrotic agent is a protein kinase B inhibitor.

12031. The method of item 11940 wherein the anti-fibrotic agent is a protein kinase C stimulant.

12032. The method of item 11940 wherein the anti-fibrotic agent is a purine nucleoside analogue.

12033. The method of item 11940 wherein the anti-fibrotic agent is a purinoreceptor P2X antagonist.

12034. The method of item 11940 wherein the anti-fibrotic agent is a Raf kinase inhibitor.

12035. The method of item 11940 wherein the anti-fibrotic agent is a reversible inhibitor of ErbB1 and ErbB2.

12036. The method of item 11940 wherein the anti-fibrotic agent is a ribonucleoside triphosphate reductase inhibitor.

12037. The method of item 11940 wherein the anti-fibrotic agent is an SDF-1 antagonist.

12038. The method of item 11940 wherein the anti-fibrotic agent is a sheddase inhibitor.

12039. The method of item 11940 wherein the anti-fibrotic agent is an SRC inhibitor.

12040. The method of item 11940 wherein the anti-fibrotic agent is a stromelysin inhibitor.

12041. The method of item 11940 wherein the anti-fibrotic agent is an Syk kinase inhibitor.

12042. The method of item 11940 wherein the anti-fibrotic agent is a telomerase inhibitor.

12043. The method of item 11940 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

12044. The method of item 11940 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

12045. The method of item 11940 wherein the anti-fibrotic agent is a Toll receptor inhibitor.

12046. The method of item 11940 wherein the anti-fibrotic agent is a tubulin antagonist.

12047. The method of item 11940 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

12048. The method of item 11940 wherein the anti-fibrotic agent is a VEGF inhibitor.

12049. The method of item 11940 wherein the anti-fibrotic agent is a vitamin D receptor agonist.

12050. The method of item 11940 wherein the anti-fibrotic agent is ZD-6474 (an angiogenesis inhibitor).

12051. The method of item 11940 wherein the anti-fibrotic agent is AP-23573 (an mTOR inhibitor).

12052. The method of item 11940 wherein the anti-fibrotic agent is synthadotin (a tubulin antagonist).

12053. The method of item 11940 wherein the anti-fibrotic agent is S-0885 (a collagenase inhibitor).

12054. The method of item 11940 wherein the anti-fibrotic agent is aplidine (an elongation factor-1 alpha inhibitor).

12055. The method of item 11940 wherein the anti-fibrotic agent is ixabepilone (an epithilone).

12056. The method of item 11940 wherein the anti-fibrotic agent is IDN-5390 (an angiogenesis inhibitor).

12057. The method of item 11940 wherein the anti-fibrotic agent is SB-2723005 (an angiogenesis inhibitor).

12058. The method of item 11940 wherein the anti-fibrotic agent is ABT-518 (an angiogenesis inhibitor).

12059. The method of item 11940 wherein the anti-fibrotic agent is combretastatin (an angiogenesis inhibitor).

12060. The method of item 11940 wherein the anti-fibrotic agent is anecortave acetate (an angiogenesis inhibitor).

12061. The method of item 11940 wherein the anti-fibrotic agent is SB-715992 (a kinesin antagonist).

12062. The method of item 11940 wherein the anti-fibrotic agent is temsirolimus (an mTOR inhibitor).

12063. The method of item 11940 wherein the anti-fibrotic agent is adalimumab (a TNFα antagonist).

12064. The method of item 11940 wherein the anti-infective agent is an anthracycline.

12065. The method of item 11940 wherein the anti-infective agent is doxorubicin.

12066. The method of item 11940 wherein the anti-infective agent is mitoxantrone.

12067. The method of item 11940 wherein the anti-infective agent is a fluoropyrimidine.

12068. The method of item 11940 wherein the anti-infective agent is 5-fluorouracil (5-FU).

12069. The method of item 11940 wherein the anti-infective agent is a folic acid antagonist.

12070. The method of item 11940 wherein the anti-infective agent is methotrexate.

12071. The method of item 11940 wherein the anti-infective agent is a podophylotoxin.

12072. The method of item 11940 wherein the anti-infective agent is etoposide.

12073. The method of item 11940 wherein the anti-infective agent is camptothecin.

12074. The method of item 11940 wherein the anti-infective agent is a hydroxyurea.

12075. The method of item 11940 wherein the anti-infective agent is a platinum complex.

12076. The method of item 11940 wherein the anti-infective agent is cisplatin.

12077. The method of item 11940 wherein the composition comprises an anti-thrombotic agent.

12078. The method of item 11940 wherein the polymer is formed from reactants comprising a naturally occurring polymer.

12079. The method of item 11940 wherein the polymer is formed from reactants comprising protein.

12080. The method of item 11940 wherein the polymer is formed from reactants comprising carbohydrate.

12081. The method of item 11940 wherein the polymer is formed from reactants comprising biodegradable polymer.

12082. The method of item 11940 wherein the polymer is formed from reactants comprising nonbiodegradable polymer.

12083. The method of item 11940 wherein the polymer is formed from reactants comprising collagen.

12084. The method of item 11940 wherein the polymer is formed from reactants comprising methylated collagen.

12085. The method of item 11940 wherein the polymer is formed from reactants comprising fibrinogen.

12086. The method of item 11940 wherein the polymer is formed from reactants comprising thrombin.

12087. The method of item 11940 wherein the polymer is formed from reactants comprising blood plasma.

12088. The method of item 11940 wherein the polymer is formed from reactants comprising calcium salt.

12089. The method of item 11940 wherein the polymer is formed from reactants comprising an antifibronolytic agent.

12090. The method of item 11940 wherein the polymer is formed from reactants comprising fibrinogen analog.

12091. The method of item 11940 wherein the polymer is formed from reactants comprising albumin.

12092. The method of item 11940 wherein the polymer is formed from reactants comprising plasminogen.

12093. The method of item 11940 wherein the polymer is formed from reactants comprising von Willebrands factor.

12094. The method of item 11940 wherein the polymer is formed from reactants comprising Factor VIII.

12095. The method of item 11940 wherein the polymer is formed from reactants comprising hypoallergenic collagen.

12096. The method of item 11940 wherein the polymer is formed from reactants comprising atelopeptidic collagen.

12097. The method of item 11940 wherein the polymer is formed from reactants comprising telopeptide collagen.

12098. The method of item 11940 wherein the polymer is formed from reactants comprising crosslinked collagen.

12099. The method of item 11940 wherein the polymer is formed from reactants comprising aprotinin.

12100. The method of item 11940 wherein the polymer is formed from reactants comprising epsilon-amino-n-caproic acid.

12101. The method of item 11940 wherein the polymer is formed from reactants comprising gelatin.

12102. The method of item 11940 wherein the polymer is formed from reactants comprising protein conjugates.

12103. The method of item 11940 wherein the polymer is formed from reactants comprising gelatin conjugates.

12104. The method of item 11940 wherein the polymer is formed from reactants comprising a synthetic polymer.

12105. The method of item 11940 wherein the polymer is formed from reactants comprising a synthetic isocyanate-containing compound.

12106. The method of item 11940 wherein the polymer is formed from reactants comprising a synthetic thiol-containing compound.

12107. The method of item 11940 wherein the polymer is formed from reactants comprising a synthetic compound containing at least two thiol groups.

12108. The method of item 11940 wherein the polymer is formed from reactants comprising a synthetic compound containing at least three thiol groups.

12109. The method of item 11940 wherein the polymer is formed from reactants comprising a synthetic compound containing at least four thiol groups.

12110. The method of item 11940 wherein the polymer is formed from reactants comprising a synthetic amino-containing compound.

12111. The method of item 11940 wherein the polymer is formed from reactants comprising a synthetic compound containing at least two amino groups.

12112. The method of item 11940 wherein the polymer is formed from reactants comprising a synthetic compound containing at least three amino groups.

12113. The method of item 11940 wherein the polymer is formed from reactants comprising a synthetic compound containing at least four amino groups.

12114. The method of item 11940 wherein the polymer is formed from reactants comprising a synthetic compound comprising a carbonyl-oxygen-succinimidyl group.

12115. The method of item 11940 wherein the polymer is formed from reactants comprising a synthetic compound comprising at least two carbonyl-oxygen-succinimidyl groups.

12116. The method of item 11940 wherein the polymer is formed from reactants comprising a synthetic compound comprising at least three carbonyl-oxygen-succinimidyl groups.

12117. The method of item 11940 wherein the polymer is formed from reactants comprising a synthetic compound comprising at least four carbonyl-oxygen-succinimidyl groups.

12118. The method of item 11940 wherein the polymer is formed from reactants comprising a synthetic polyalkylene oxide-containing compound.

12119. The method of item 11940 wherein the polymer is formed from reactants comprising a synthetic compound comprising both polyalkylene oxide and biodegradable polyester blocks.

12120. The method of item 11940 wherein the polymer is formed from reactants comprising a synthetic polyalkylene oxide-containing compound having reactive amino groups.

12121. The method of item 11940 wherein the polymer is formed from reactants comprising a synthetic polyalkylene oxide-containing compound having reactive thiol groups.

12122. The method of item 11940 wherein the polymer is formed from reactants comprising a synthetic polyalkylene oxide-containing compound having reactive carbonyl-oxygen-succinimidyl groups.

12123. The method of item 11940 wherein the polymer is formed from reactants comprising a synthetic compound comprising a biodegradable polyester block.

12124. The method of item 11940 wherein the polymer is formed from reactants comprising a synthetic polymer formed in whole or part from lactic acid or lactide.

12125. The method of item 11940 wherein the polymer is formed from reactants comprising a synthetic polymer formed in whole or part from glycolic acid or glycolide.

12126. The method of item 11940 wherein the polymer is formed from reactants comprising polylysine.

12127. The method of item 11940 wherein the polymer is formed from reactants comprising (a) protein and (b) a compound comprising a polyalkylene oxide portion.

12128. The method of item 11940 wherein the polymer is formed from reactants comprising (a) protein and (b) polylysine.

12129. The method of item 11940 wherein the polymer is formed from reactants comprising (a) protein and (b) a compound having at least four thiol groups.

12130. The method of item 11940 wherein the polymer is formed from reactants comprising (a) protein and (b) a compound having at least four amino groups.

12131. The method of item 11940 wherein the polymer is formed from reactants comprising (a) protein and (b) a compound having at least four carbonyl-oxygen-succinimide groups.

12132. The method of item 11940 wherein the polymer is formed from reactants comprising (a) protein and (b) a compound having a biodegradable region formed from reactants selected from lactic acid, lactide, glycolic acid, glycolide, and epison-caprolactone.

12133. The method of item 11940 wherein the polymer is formed from reactants comprising (a) collagen and (b) a compound comprising a polyalkylene oxide portion.

12134. The method of item 11940 wherein the polymer is formed from reactants comprising (a) collagen and (b) polylysine.

12135. The method of item 11940 wherein the polymer is formed from reactants comprising (a) collagen and (b) a compound having at least four thiol groups.

12136. The method of item 11940 wherein the polymer is formed from reactants comprising (a) collagen and (b) a compound having at least four amino groups.

12137. The method of item 11940 wherein the polymer is formed from reactants comprising (a) collagen and (b) a compound having at least four carbonyl-oxygen-succinimide groups.

12138. The method of item 11940 wherein the polymer is formed from reactants comprising (a) collagen and (b) a compound having a biodegradable region formed from reactants selected from lactic acid, lactide, glycolic acid, glycolide, and epison-caprolactone.

12139. The method of item 11940 wherein the polymer is formed from reactants comprising (a) methylated collagen and (b) a compound comprising a polyalkylene oxide portion.

12140. The method of item 11940 wherein the polymer is formed from reactants comprising (a) methylated collagen and (b) polylysine.

12141. The method of item 11940 wherein the polymer is formed from reactants comprising (a) methylated collagen and (b) a compound having at least four thiol groups.

12142. The method of item 11940 wherein the polymer is formed from reactants comprising (a) methylated collagen and (b) a compound having at least four amino groups.

12143. The method of item 11940 wherein the polymer is formed from reactants comprising (a) methylated collagen and (b) a compound having at least four carbonyl-oxygen-succinimide groups.

12144. The method of item 11940 wherein the polymer is formed from reactants comprising (a) methylated collagen and (b) a compound having a biodegradable region formed from reactants selected from lactic acid, lactide, glycolic acid, glycolide, and epison-caprolactone.

12145. The method of item 11940 wherein the polymer is formed from reactants comprising hyaluronic acid.

12146. The method of item 11940 wherein the polymer is formed from reactants comprising a hyaluronic acid derivative.

12147. The method of item 11940 wherein the polymer is formed from reactants comprising pentaerythritol poly(ethylene glycol)ether tetra-sulfhydryl of number average molecular weight between 3,000 and 30,000.

12148. The method of item 11940 wherein the polymer is formed from reactants comprising pentaerythritol poly(ethylene glycol)ether tetra-amino of number average molecular weight between 3,000 and 30,000.

12149. The method of item 11940 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

12150. The method of item 11940 wherein the composition comprises a colorant.

12151. The method of item 11940 wherein the composition is sterile.

12152. The method for implanting an electrical device of any one of items 11940-12151 wherein the neurostimulator comprises an intracranially implantable electrical control module and an electrode.

12153. The method for implanting an electrical device of any one of items 11940-12151 wherein the neurostimulator comprises a sensor and an electrode.

12154 A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a vagal nerve stimulator for treating epilepsy.

12155. The method of item 12154 comprising: (a) infiltrating a tissue of a host where the electrical device is to be, or has been, implanted with an anti-fibrotic agent, and (b) implanting the electrical device into the host.

12156. The method of item 12154 comprising: (a) infiltrating a tissue of a host where the electrical device is to be, or has been, implanted with an anti-infective agent, and (b) implanting the electrical device into the host

12157. The method of item 12154 comprising: (a) infiltrating a tissue of a host where the electrical device is to be, or has been, implanted with a polymer; and (b) implanting the electrical device into the host.

12158. The method of item 12154 comprising: (a) infiltrating a tissue of a host where the electrical device is to be, or has been, implanted with a composition comprising an anti-fibrotic agent and a polymer, and (b) implanting the electrical device into the host

12159. The method of item 12154 comprising: (a) infiltrating a tissue of a host where the electrical device is to be, or has been, implanted with a composition comprising an anti-infective agent and a polymer, and (b) implanting the electrical device into the host.

12160. The method of item 12154 comprising: (a) infiltrating a tissue of a host where the electrical device is to be, or has been, implanted with a composition comprising an anti-fibrotic agent, an anti-infective agent and a polymer, and (b) implanting the electrical device into the host.

12161. The method of item 12154 wherein the agent is an adensosine A2A receptor antagonist.

12162. The method of item 12154 wherein the anti-fibrotic agent is an AKT inhibitor.

12163. The method of item 12154 wherein the anti-fibrotic agent is an alpha 2 integrin antagonist, wherein the alpha 2 integrin antagonist is Pharmaprojects No. 5754 (Merck KgaA).

12164. The method of item 12154 wherein the anti-fibrotic agent is an alpha 4 integrin antagonist.

12165. The method of item 12154 wherein the anti-fibrotic agent is an alpha 7 nicotinic receptor agonist.

12166. The method of item 12154 wherein the anti-fibrotic agent is an angiogenesis inhibitor selected from the group consisting of AG-12,958 (Pfizer), ATN-161 (Aftenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

12167. The method of item 12154 wherein the anti-fibrotic agent is an apoptosis antagonist.

12168. The method of item 12154 wherein the anti-fibrotic agent is an apoptosis activator.

12169. The method of item 12154 wherein the anti-fibrotic agent is a beta 1 integrin antagonist.

12170. The method of item 12154 wherein the anti-fibrotic agent is a beta tubulin inhibitor.

12171. The method of item 12154 wherein the anti-fibrotic agent is a blocker of enzyme production in Hepatitis C.

12172. The method of item 12154 wherein the anti-fibrotic agent is a Bruton's tyrosine kinase inhibitor.

12173. The method of item 12154 wherein the anti-fibrotic agent is a calcineurin inhibitor.

12174. The method of item 12154 wherein the anti-fibrotic agent is a caspase 3 inhibitor.

12175. The method of item 12154 wherein the anti-fibrotic agent is a CC chemokine receptor antagonist.

12176. The method of item 12154 wherein the anti-fibrotic agent is a cell cycle inhibitor selected from the group consisting of SNS-595 (Sunesis), synthadotin, KRX-0403, and an analogue or derivative thereof.

12177. The method of item 12154 wherein the anti-fibrotic agent is a cathepsin B inhibitor.

12178. The method of item 12154 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

12179. The method of item 12154 wherein the anti-fibrotic agent is a cathepsin L inhibitor.

12180. The method of item 12154 wherein the anti-fibrotic agent is a CD40 antagonist.

12181. The method of item 12154 wherein the anti-fibrotic agent is a chemokine receptor agonist.

12182. The method of item 12154 wherein the anti-fibrotic agent is a chymase inhibitor.

12183. The method of item 12154 wherein the anti-fibrotic agent is a collagenase antagonist.

12184. The method of item 12154 wherein the anti-fibrotic agent is a CXCR antagonist.

12185. The method of item 12154 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

12186. The method of item 12154 wherein the anti-fibrotic agent is a cyclooxygenase 1 inhibitor.

12187. The method of item 12154 wherein the anti-fibrotic agent is a DHFR inhibitor.

12188. The method of item 12154 wherein the anti-fibrotic agent is a dual integrin inhibitor.

12189. The method of item 12154 wherein the anti-fibrotic agent is an elastase inhibitor.

12190. The method of item 12154 wherein the anti-fibrotic agent is an elongation factor-1 alpha inhibitor.

12191. The method of item 12154 wherein the anti-fibrotic agent is an endothelial growth factor antagonist.

12192. The method of item 12154 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

12193. The method of item 12154 wherein the anti-fibrotic agent is an endotoxin antagonist.

12194. The method of item 12154 wherein the anti-fibrotic agent is an epothilone and tubulin binder.

12195. The method of item 12154 wherein the anti-fibrotic agent is an estrogen receptor antagonist.

12196. The method of item 12154 wherein the anti-fibrotic agent is an FGF inhibitor.

12197. The method of item 12154 wherein the anti-fibrotic agent is a farnexyl transferase inhibitor.

12198. The method of item 12154 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

12199. The method of item 12154 wherein the anti-fibrotic agent is an FLT-3 kinase inhibitor.

12200. The method of item 12154 wherein the anti-fibrotic agent is an FGF receptor kinase inhibitor.

12201. The method of item 12154 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

12202. The method of item 12154 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

12203. The method of item 12154 wherein the anti-fibrotic agent is a histone deacetylase inhibitor.

12204. The method of item 12154 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

12205. The method of item 12154 wherein the anti-fibrotic agent is an ICAM inhibitor.

12206. The method of item 12154 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

12207. The method of item 12154 wherein the anti-fibrotic agent is an IL-2 inhibitor.

12208. The method of item 12154 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

12209. The method of item 12154 wherein the anti-fibrotic agent is an IMPDH (inosine monophosphate).

12210. The method of item 12154 wherein the anti-fibrotic agent is an integrin antagonist.

12211. The method of item 12154 wherein the anti-fibrotic agent is an interleukin antagonist.

12212. The method of item 12154 wherein the anti-fibrotic agent is an inhibitor of type III receptor tyrosine kinase.

12213. The method of item 12154 wherein the anti-fibrotic agent is an irreversible inhibitor of enzyme methionine aminopeptidase type 2.

12214. The method of item 12154 wherein the anti-fibrotic agent is an isozyme selective delta protein kinase C inhibitor.

12215. The method of item 12154 wherein the anti-fibrotic agent a JAK3 enzyme inhibitor.

12216. The method of item 12154 wherein the anti-fibrotic agent is a JNK inhibitor.

12217. The method of item 12154 wherein the anti-fibrotic agent is a kinase inhibitor.

12218. The method of item 12154 wherein the anti-fibrotic agent is kinesin antagonist.

12219. The method of item 12154 wherein the anti-fibrotic agent is a kinesin antagonist.

12220. The method of item 12154 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

12221. The method of item 12154 wherein the anti-fibrotic agent is an MAP kinase inhibitor.

12222. The method of item 12154 wherein the anti-fibrotic agent is a matrix metalloproteinase inhibitor.

12223. The method of item 12154 wherein the anti-fibrotic agent is an MCP-CCR2 inhibitor.

12224. The method of item 12154 wherein the anti-fibrotic agent is an mTOR inhibitor.

12225. The method of item 12154 wherein the anti-fibrotic agent is an mTOR kinase inhibitor.

12226. The method of item 12154 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

12227. The method of item 12154 wherein the anti-fibrotic agent is an MIF inhibitor.

12228. The method of item 12154 wherein the anti-fibrotic agent is an MMP inhibitor.

12229. The method of item 12154 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

12230. The method of item 12154 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

12231. The method of item 12154 wherein the anti-fibrotic agent is a nitric oxide agonist.

12232. The method of item 12154 wherein the anti-fibrotic agent is an ornithine decarboxylase inhibitor.

12233. The method of item 12154 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

12234. The method of item 12154 wherein the anti-fibrotic agent is a palmitoyl-protein thioesterase inhibitor.

12235. The method of item 12154 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

12236. The method of item 12154 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

12237. The method of item 12154 wherein the anti-fibrotic agent is a phosphatase inhibitor.

12238. The method of item 12154 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-1 4-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

12239. The method of item 12154 wherein the anti-fibrotic agent is a PKC inhibitor.

12240. The method of item 12154 wherein the anti-fibrotic agent is a platelet activating factor antagonist.

12241. The method of item 12154 wherein the anti-fibrotic agent is a platelet-derived growth factor receptor kinase inhibitor.

12242. The method of item 12154 wherein the anti-fibrotic agent is a prolyl hydroxylase inhibitor.

12243. The method of item 12154 wherein the anti-fibrotic agent is a polymorphonuclear neutrophil inhibitor.

12244. The method of item 12154 wherein the anti-fibrotic agent is a protein kinase B inhibitor.

12245. The method of item 12154 wherein the anti-fibrotic agent is a protein kinase C stimulant.

12246. The method of item 12154 wherein the anti-fibrotic agent is a purine nucleoside analogue.

12247. The method of item 12154 wherein the anti-fibrotic agent is a purinoreceptor P2X antagonist.

12248. The method of item 12154 wherein the anti-fibrotic agent is a Raf kinase inhibitor.

12249. The method of item 12154 wherein the anti-fibrotic agent is a reversible inhibitor of ErbB1 and ErbB2.

12250. The method of item 12154 wherein the anti-fibrotic agent is a ribonucleoside triphosphate reductase inhibitor.

12251. The method of item 12154 wherein the anti-fibrotic agent is an SDF-1 antagonist.

12252. The method of item 12154 wherein the anti-fibrotic agent is a sheddase inhibitor.

12253. The method of item 12154 wherein the anti-fibrotic agent is an SRC inhibitor.

12254. The method of item 12154 wherein the anti-fibrotic agent is a stromelysin inhibitor.

12255. The method of item 12154 wherein the anti-fibrotic agent is an Syk kinase inhibitor.

12256. The method of item 12154 wherein the anti-fibrotic agent is a telomerase inhibitor.

12257. The method of item 12154 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

12258. The method of item 12154 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-214) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

12259. The method of item 12154 wherein the anti-fibrotic agent is a Toll receptor inhibitor.

12260. The method of item 12154 wherein the anti-fibrotic agent is a tubulin antagonist.

12261. The method of item 12154 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-10 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

12262. The method of item 12154 wherein the anti-fibrotic agent is a VEGF inhibitor.

12263. The method of item 12154 wherein the anti-fibrotic agent is a vitamin D receptor agonist.

12264. The method of item 12154 wherein the anti-fibrotic agent is ZD-6474 (an angiogenesis inhibitor).

12265. The method of item 12154 wherein the anti-fibrotic agent is AP-23573 (an mTOR inhibitor).

12266. The method of item 12154 wherein the anti-fibrotic agent is synthadotin (a tubulin antagonist).

12267. The method of item 12154 wherein the anti-fibrotic agent is S-0885 (a collagenase inhibitor).

12268. The method of item 12154 wherein the anti-fibrotic agent is aplidine (an elongation factor-1 alpha inhibitor).

12269. The method of item 12154 wherein the anti-fibrotic agent is ixabepilone (an epithilone).

12270. The method of item 12154 wherein the anti-fibrotic agent is IDN-5390 (an angiogenesis inhibitor).

12271. The method of item 12154 wherein the anti-fibrotic agent is SB-2723005 (an angiogenesis inhibitor).

12272. The method of item 12154 wherein the anti-fibrotic agent is ABT-518 (an angiogenesis inhibitor).

12273. The method of item 12154 wherein the anti-fibrotic agent is combretastatin (an angiogenesis inhibitor).

12274. The method of item 12154 wherein the anti-fibrotic agent is anecortave acetate (an angiogenesis inhibitor).

12275. The method of item 12154 wherein the anti-fibrotic agent is SB-715992 (a kinesin antagonist).

12276. The method of item 12154 wherein the anti-fibrotic agent is temsirolimus (an mTOR inhibitor).

12277. The method of item 12154 wherein the anti-fibrotic agent is adalimumab (a TNFα antagonist).

12278. The method of item 12154 wherein the anti-infective agent is an anthracycline.

12279. The method of item 12154 wherein the anti-infective agent is doxorubicin.

12280. The method of item 12154 wherein the anti-infective agent is mitoxantrone.

12281. The method of item 12154 wherein the anti-infective agent is a fluoropyrimidine.

12282. The method of item 12154 wherein the anti-infective agent is 5-fluorouracil (5-FU).

12283. The method of item 12154 wherein the anti-infective agent is a folic acid antagonist.

12284. The method of item 12154 wherein the anti-infective agent is methotrexate.

12285. The method of item 12154 wherein the anti-infective agent is a podophylotoxin.

12286. The method of item 12154 wherein the anti-infective agent is etoposide.

12287. The method of item 12154 wherein the anti-infective agent is camptothecin.

12288. The method of item 12154 wherein the anti-infective agent is a hydroxyurea.

12289. The method of item 12154 wherein the anti-infective agent is a platinum complex.

12290. The method of item 12154 wherein the anti-infective agent is cisplatin.

12291. The method of item 12154 wherein the composition comprises an anti-thrombotic agent.

12292. The method of item 12154 wherein the polymer is formed from reactants comprising a naturally occurring polymer.

12293. The method of item 12154 wherein the polymer is formed from reactants comprising protein.

12294. The method of item 12154 wherein the polymer is formed from reactants comprising carbohydrate.

12295. The method of item 12154 wherein the polymer is formed from reactants comprising biodegradable polymer.

12296. The method of item 12154 wherein the polymer is formed from reactants comprising nonbiodegradable polymer.

12297. The method of item 12154 wherein the polymer is formed from reactants comprising collagen.

12298. The method of item 12154 wherein the polymer is formed from reactants comprising methylated collagen.

12299. The method of item 12154 wherein the polymer is formed from reactants comprising fibrinogen.

12300. The method of item 12154 wherein the polymer is formed from reactants comprising thrombin.

12301. The method of item 12154 wherein the polymer is formed from reactants comprising blood plasma.

12302. The method of item 12154 wherein the polymer is formed from reactants comprising calcium salt.

12303. The method of item 12154 wherein the polymer is formed from reactants comprising an antifibronolytic agent.

12304. The method of item 12154 wherein the polymer is formed from reactants comprising fibrinogen analog.

12305. The method of item 12154 wherein the polymer is formed from reactants comprising albumin.

12306. The method of item 12154 wherein the polymer is formed from reactants comprising plasminogen.

12307. The method of item 12154 wherein the polymer is formed from reactants comprising von Willebrands factor.

12308. The method of item 12154 wherein the polymer is formed from reactants comprising Factor VIII.

12309. The method of item 12154 wherein the polymer is formed from reactants comprising hypoallergenic collagen.

12310. The method of item 12154 wherein the polymer is formed from reactants comprising atelopeptidic collagen.

12311. The method of item 12154 wherein the polymer is formed from reactants comprising telopeptide collagen.

12312. The method of item 12154 wherein the polymer is formed from reactants comprising crosslinked collagen.

12313. The method of item 12154 wherein the polymer is formed from reactants comprising aprotinin.

12314. The method of item 12154 wherein the polymer is formed from reactants comprising epsilon-amino-n-caproic acid.

12315. The method of item 12154 wherein the polymer is formed from reactants comprising gelatin.

12316. The method of item 12154 wherein the polymer is formed from reactants comprising protein conjugates.

12317. The method of item 12154 wherein the polymer is formed from reactants comprising gelatin conjugates.

12318. The method of item 12154 wherein the polymer is formed from reactants comprising a synthetic polymer.

12319. The method of item 12154 wherein the polymer is formed from reactants comprising a synthetic isocyanate-containing compound.

12320. The method of item 12154 wherein the polymer is formed from reactants comprising a synthetic thiol-containing compound.

12321. The method of item 12154 wherein the polymer is formed from reactants comprising a synthetic compound containing at least two thiol groups.

12322. The method of item 12154 wherein the polymer is formed from reactants comprising a synthetic compound containing at least three thiol groups.

12323. The method of item 12154 wherein the polymer is formed from reactants comprising a synthetic compound containing at least four thiol groups.

12324. The method of item 12154 wherein the polymer is formed from reactants comprising a synthetic amino-containing compound

12325. The method of item 12154 wherein the polymer is formed from reactants comprising a synthetic compound containing at least two amino groups.

12326. The method of item 12154 wherein the polymer is formed from reactants comprising a synthetic compound containing at least three amino groups.

12327. The method of item 12154 wherein the polymer is formed from reactants comprising a synthetic compound containing at least four amino groups.

12328. The method of item 12154 wherein the polymer is formed from reactants comprising a synthetic compound comprising a carbonyl-oxygen-succinimidyl group.

12329. The method of item 12154 wherein the polymer is formed from reactants comprising a synthetic compound comprising at least two carbonyl-oxygen-succinimidyl groups.

12330. The method of item 12154 wherein the polymer is formed from reactants comprising a synthetic compound comprising at least three carbonyl-oxygen-succinimidyl groups.

12331. The method of item 12154 wherein the polymer is formed from reactants comprising a synthetic compound comprising at least four carbonyl-oxygen-succinimidyl groups.

12332. The method of item 12154 wherein the polymer is formed from reactants comprising a synthetic polyalkylene oxide-containing compound.

12333. The method of item 12154 wherein the polymer is formed from reactants comprising a synthetic compound comprising both polyalkylene oxide and biodegradable polyester blocks.

12334. The method of item 12154 wherein the polymer is formed from reactants comprising a synthetic polyalkylene oxide-containing compound having reactive amino groups.

12335. The method of item 12154 wherein the polymer is formed from reactants comprising a synthetic polyalkylene oxide-containing compound having reactive thiol groups.

12336. The method of item 12154 wherein the polymer is formed from reactants comprising a synthetic polyalkylene oxide-containing compound having reactive carbonyl-oxygen-succinimidyl groups.

12337. The method of item 12154 wherein the polymer is formed from reactants comprising a synthetic compound comprising a biodegradable polyester block.

12338. The method of item 12154 wherein the polymer is formed from reactants comprising a synthetic polymer formed in whole or part from lactic acid or lactide.

12339. The method of item 12154 wherein the polymer is formed from reactants comprising a synthetic polymer formed in whole or part from glycolic acid or glycolide.

12340. The method of item 12154 wherein the polymer is formed from reactants comprising polylysine.

12341. The method of item 12154 wherein the polymer is formed from reactants comprising (a) protein and (b) a compound comprising a polyalkylene oxide portion.

12342. The method of item 12154 wherein the polymer is formed from reactants comprising (a) protein and (b) polylysine.

12343. The method of item 12154 wherein the polymer is formed from reactants comprising (a) protein and (b) a compound having at least four thiol groups.

12344. The method of item 12154 wherein the polymer is formed from reactants comprising (a) protein and (b) a compound having at least four amino groups.

12345. The method of item 12154 wherein the polymer is formed from reactants comprising (a) protein and (b) a compound having at least four carbonyl-oxygen-succinimide groups.

12346. The method of item 12154 wherein the polymer is formed from reactants comprising (a) protein and (b) a compound having a biodegradable region formed from reactants selected from lactic acid, lactide, glycolic acid, glycolide, and epison-caprolactone.

12347. The method of item 12154 wherein the polymer is formed from reactants comprising (a) collagen and (b) a compound comprising a polyalkylene oxide portion.

12348. The method of item 12154 wherein the polymer is formed from reactants comprising (a) collagen and (b) polylysine.

12349. The method of item 12154 wherein the polymer is formed from reactants comprising (a) collagen and (b) a compound having at least four thiol groups.

12350. The method of item 12154 wherein the polymer is formed from reactants comprising (a) collagen and (b) a compound having at least four amino groups.

12351. The method of item 12154 wherein the polymer is formed from reactants comprising (a) collagen and (b) a compound having at least four carbonyl-oxygen-succinimide groups.

12352. The method of item 12154 wherein the polymer is formed from reactants comprising (a) collagen and (b) a compound having a biodegradable region formed from reactants selected from lactic acid, lactide, glycolic acid, glycolide, and epison-caprolactone.

12353. The method of item 12154 wherein the polymer is formed from reactants comprising (a) methylated collagen and (b) a compound comprising a polyalkylene oxide portion.

12354. The method of item 12154 wherein the polymer is formed from reactants comprising (a) methylated collagen and (b) polylysine.

12355. The method of item 12154 wherein the polymer is formed from reactants comprising (a) methylated collagen and (b) a compound having at least four thiol groups.

12356. The method of item 12154 wherein the polymer is formed from reactants comprising (a) methylated collagen and (b) a compound having at least four amino groups.

12357. The method of item 12154 wherein the polymer is formed from reactants comprising (a) methylated collagen and (b) a compound having at least four carbonyl-oxygen-succinimide groups.

12358. The method of item 12154 wherein the polymer is formed from reactants comprising (a) methylated collagen and (b) a compound having a biodegradable region formed from reactants selected from lactic acid, lactide, glycolic acid, glycolide, and epison-caprolactone.

12359. The method of item 12154 wherein the polymer is formed from reactants comprising hyaluronic acid.

12360. The method of item 12154 wherein the polymer is formed from reactants comprising a hyaluronic acid derivative.

12361. The method of item 12154 wherein the polymer is formed from reactants comprising pentaerythritol poly(ethylene glycol)ether tetra-sulfhydryl of number average molecular weight between 3,000 and 30,000.

12362. The method of item 12154 wherein the polymer is formed from reactants comprising pentaerythritol poly(ethylene glycol)ether tetra-amino of number average molecular weight between 3,000 and 30,000.

12363. The method of item 12154 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

12364. The method of item 12154 wherein the composition comprises a colorant.

12365. The method of item 12154 wherein the composition is sterile.

12366. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a vagal nerve stimulator.

12367. The method of item 12366 comprising: (a) infiltrating a tissue of a host where the electrical device is to be, or has been, implanted with an anti-fibrotic agent, and (b) implanting the electrical device into the host.

12368. The method of item 12366 comprising: (a) infiltrating a tissue of a host where the electrical device is to be, or has been, implanted with an anti-infective agent, and (b) implanting the electrical device into the host.

12369. The method of item 12366 comprising: (a) infiltrating a tissue of a host where the electrical device is to be, or has been, implanted with a polymer; and (b) implanting the electrical device into the host.

12370. The method of item 12366 comprising: (a) infiltrating a tissue of a host where the electrical device is to be, or has been, implanted with a composition comprising an anti-fibrotic agent and a polymer, and (b) implanting the electrical device into the host.

12371. The method of item 12366 comprising: (a) infiltrating a tissue of a host where the electrical device is to be, or has been, implanted with a composition comprising an anti-infective agent and a polymer, and (b) implanting the electrical device into the host.

12372. The method of item 12366 comprising: (a) infiltrating a tissue of a host where the electrical device is to be, or has been, implanted with a composition comprising an anti-fibrotic agent, an anti-infective agent and a polymer, and (b) implanting the electrical device into the host.

12373. The method of item 12366 wherein the agent is an adensosine A2A receptor antagonist.

12374. The method of item 12366 wherein the anti-fibrotic agent is an AKT inhibitor.

12375. The method of item 12366 wherein the anti-fibrotic agent is an alpha 2 integrin antagonist, wherein the alpha 2 integrin antagonist is Pharmaprojects No. 5754 (Merck KgaA).

12376. The method of item 12366 wherein the anti-fibrotic agent is an alpha 4 integrin antagonist.

12377. The method of item 12366 wherein the anti-fibrotic agent is an alpha 7 nicotinic receptor agonist.

12378. The method of item 12366 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

12379. The method of item 12366 wherein the anti-fibrotic agent is an apoptosis antagonist.

12380. The method of item 12366 wherein the anti-fibrotic agent is an apoptosis activator.

12381. The method of item 12366 wherein the anti-fibrotic agent is a beta 1 integrin antagonist.

12382. The method of item 12366 wherein the anti-fibrotic agent is a beta tubulin inhibitor.

12383. The method of item 12366 wherein the anti-fibrotic agent is a blocker of enzyme production in Hepatitis C.

12384. The method of item 12366 wherein the anti-fibrotic agent is a Bruton's tyrosine kinase inhibitor.

12385. The method of item 12366 wherein the anti-fibrotic agent is a calcineurin inhibitor.

12386. The method of item 12366 wherein the anti-fibrotic agent is a caspase 3 inhibitor.

12387. The method of item 12366 wherein the anti-fibrotic agent is a CC chemokine receptor antagonist.

12388. The method of item 12366 wherein the anti-fibrotic agent is a cell cycle inhibitor selected from the group consisting of SNS-595 (Sunesis), synthadotin, KRX-0403, and an analogue or derivative thereof.

12389. The method of item 12366 wherein the anti-fibrotic agent is a cathepsin B inhibitor.

12390. The method of item 12366 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof

12391. The method of item 12366 wherein the anti-fibrotic agent is a cathepsin L inhibitor.

12392. The method of item 12366 wherein the anti-fibrotic agent is a CD40 antagonist.

12393. The method of item 12366 wherein the anti-fibrotic agent is a chemokine receptor agonist.

12394. The method of item 12366 wherein the anti-fibrotic agent is a chymase inhibitor.

12395. The method of item 12366 wherein the anti-fibrotic agent is a collagenase antagonist.

12396. The method of item 12366 wherein the anti-fibrotic agent is a CXCR antagonist.

12397. The method of item 12366 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

12398. The method of item 12366 wherein the anti-fibrotic agent is a cyclooxygenase 1 inhibitor.

12399. The method of item 12366 wherein the anti-fibrotic agent is a DHFR inhibitor.

12400. The method of item 12366 wherein the anti-fibrotic agent is a dual integrin inhibitor.

12401. The method of item 12366 wherein the anti-fibrotic agent is an elastase inhibitor.

12402. The method of item 12366 wherein the anti-fibrotic agent is an elongation factor-1 alpha inhibitor.

12403. The method of item 12366 wherein the anti-fibrotic agent is an endothelial growth factor antagonist.

12404. The method of item 12366 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

12405. The method of item 12366 wherein the anti-fibrotic agent is an endotoxin antagonist.

12406. The method of item 12366 wherein the anti-fibrotic agent is an epothilone and tubulin binder.

12407. The method of item 12366 wherein the anti-fibrotic agent is an estrogen receptor antagonist.

12408. The method of item 12366 wherein the anti-fibrotic agent is an FGF inhibitor.

12409. The method of item 12366 wherein the anti-fibrotic agent is a farnexyl transferase inhibitor.

12410. The method of item 12366 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

12411. The method of item 12366 wherein the anti-fibrotic agent is an FLT-3 kinase inhibitor.

12412. The method of item 12366 wherein the anti-fibrotic agent is an FGF receptor kinase inhibitor.

12413. The method of item 12366 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

12414. The method of item 12366 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

12415. The method of item 12366 wherein the anti-fibrotic agent is a histone deacetylase inhibitor.

12416. The method of item 12366 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

12417. The method of item 12366 wherein the anti-fibrotic agent is an ICAM inhibitor.

12418. The method of item 12366 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

12419. The method of item 12366 wherein the anti-fibrotic agent is an IL-2 inhibitor.

12420. The method of item 12366 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

12421. The method of item 12366 wherein the anti-fibrotic agent is an IMPDH (inosine monophosphate).

12422. The method of item 12366 wherein the anti-fibrotic agent is an integrin antagonist.

12423. The method of item 12366 wherein the anti-fibrotic agent is an interleukin antagonist.

12424. The method of item 12366 wherein the anti-fibrotic agent is an inhibitor of type III receptor tyrosine kinase.

12425. The method of item 12366 wherein the anti-fibrotic agent is an irreversible inhibitor of enzyme methionine aminopeptidase type 2.

12426. The method of item 12366 wherein the anti-fibrotic agent is an isozyme selective delta protein kinase C inhibitor.

12427. The method of item 12366 wherein the anti-fibrotic agent a JAK3 enzyme inhibitor.

12428. The method of item 12366 wherein the anti-fibrotic agent is a JNK inhibitor.

12429. The method of item 12366 wherein the anti-fibrotic agent is a kinase inhibitor.

12430. The method of item 12366 wherein the anti-fibrotic agent is kinesin antagonist.

12431. The method of item 12366 wherein the anti-fibrotic agent is a kinesin antagonist.

12432. The method of item 12366 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

12433. The method of item 12366 wherein the anti-fibrotic agent is an MAP kinase inhibitor.

12434. The method of item 12366 wherein the anti-fibrotic agent is a matrix metalloproteinase inhibitor.

12435. The method of item 12366 wherein the anti-fibrotic agent is an MCP-CCR2 inhibitor.

12436. The method of item 12366 wherein the anti-fibrotic agent is an mTOR inhibitor.

12437. The method of item 12366 wherein the anti-fibrotic agent is an mTOR kinase inhibitor.

12438. The method of item 12366 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

12439. The method of item 12366 wherein the anti-fibrotic agent is an MIF inhibitor.

12440. The method of item 12366 wherein the anti-fibrotic agent is an MMP inhibitor.

12441. The method of item 12366 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

12442. The method of item 12366 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 510449-4) (Encore Pharmaceuticals), and an analogue or derivative thereof.

12443. The method of item 12366 wherein the anti-fibrotic agent is a nitric oxide agonist.

12444. The method of item 12366 wherein the anti-fibrotic agent is an ornithine decarboxylase inhibitor.

12445. The method of item 12366 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

12446. The method of item 12366 wherein the anti-fibrotic agent is a palmitoyl-protein thioesterase inhibitor.

12447. The method of item 12366 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

12448. The method of item 12366 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

12449. The method of item 12366 wherein the anti-fibrotic agent is a phosphatase inhibitor.

12450. The method of item 12366 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

12451. The method of item 12366 wherein the anti-fibrotic agent is a PKC inhibitor.

12452. The method of item 12366 wherein the anti-fibrotic agent is a platelet activating factor antagonist.

12453. The method of item 12366 wherein the anti-fibrotic agent is a platelet-derived growth factor receptor kinase inhibitor.

12454. The method of item 12366 wherein the anti-fibrotic agent is a prolyl hydroxylase inhibitor.

12455. The method of item 12366 wherein the anti-fibrotic agent is a polymorphonuclear neutrophil inhibitor.

12456. The method of item 12366 wherein the anti-fibrotic agent is a protein kinase B inhibitor.

12457. The method of item 12366 wherein the anti-fibrotic agent is a protein kinase C stimulant.

12458. The method of item 12366 wherein the anti-fibrotic agent is a purine nucleoside analogue.

12459. The method of item 12366 wherein the anti-fibrotic agent is a purinoreceptor P2X antagonist.

12460. The method of item 12366 wherein the anti-fibrotic agent is a Raf kinase inhibitor.

12461. The method of item 12366 wherein the anti-fibrotic agent is a reversible inhibitor of ErbB1 and ErbB2.

12462. The method of item 12366 wherein the anti-fibrotic agent is a ribonucleoside triphosphate reductase inhibitor.

12463. The method of item 12366 wherein the anti-fibrotic agent is an SDF-1 antagonist.

12464. The method of item 12366 wherein the anti-fibrotic agent is a sheddase inhibitor.

12465. The method of item 12366 wherein the anti-fibrotic agent is an SRC inhibitor.

12466. The method of item 12366 wherein the anti-fibrotic agent is a stromelysin inhibitor.

12467. The method of item 12366 wherein the anti-fibrotic agent is an Syk kinase inhibitor.

12468. The method of item 12366 wherein the anti-fibrotic agent is a telomerase inhibitor.

12469. The method of item 12366 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

12470. The method of item 12366 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

12471. The method of item 12366 wherein the anti-fibrotic agent is a Toll receptor inhibitor.

12472. The method of item 12366 wherein the anti-fibrotic agent is a tubulin antagonist.

12473. The method of item 12366 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-10 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

12474. The method of item 12366 wherein the anti-fibrotic agent is a VEGF inhibitor.

12475. The method of item 12366 wherein the anti-fibrotic agent is a vitamin D receptor agonist.

12476. The method of item 12366 wherein the anti-fibrotic agent is ZD-6474 (an angiogenesis inhibitor).

12477. The method of item 12366 wherein the anti-fibrotic agent is AP-23573 (an mTOR inhibitor).

12478. The method of item 12366 wherein the anti-fibrotic agent is synthadotin (a tubulin antagonist).

12479. The method of item 12366 wherein the anti-fibrotic agent is S-0885 (a collagenase inhibitor).

12480. The method of item 12366 wherein the anti-fibrotic agent is aplidine (an elongation factor-1 alpha inhibitor).

12481. The method of item 12366 wherein the anti-fibrotic agent is ixabepilone (an epithilone).

12482. The method of item 12366 wherein the anti-fibrotic agent is IDN-5390 (an angiogenesis inhibitor).

12483. The method of item 12366 wherein the anti-fibrotic agent is SB-2723005 (an angiogenesis inhibitor).

12484. The method of item 12366 wherein the anti-fibrotic agent is ABT-518 (an angiogenesis inhibitor).

12485. The method of item 12366 wherein the anti-fibrotic agent is combretastatin (an angiogenesis inhibitor).

12486. The method of item 12366 wherein the anti-fibrotic agent is anecortave acetate (an angiogenesis inhibitor).

12487. The method of item 12366 wherein the anti-fibrotic agent is SB-715992 (a kinesin antagonist).

12488. The method of item 12366 wherein the anti-fibrotic agent is temsirolimus (an mTOR inhibitor).

12489. The method of item 12366 wherein the anti-fibrotic agent is adalimumab (a TNFα antagonist).

12490. The method of item 12366 wherein the anti-infective agent is an anthracycline.

12491. The method of item 12366 wherein the anti-infective agent is doxorubicin.

12492. The method of item 12366 wherein the anti-infective agent is mitoxantrone.

12493. The method of item 12366 wherein the anti-infective agent is a fluoropyrimidine.

12494. The method of item 12366 wherein the anti-infective agent is 5-fluorouracil (5-FU).

12495. The method of item 12366 wherein the anti-infective agent is a folic acid antagonist.

12496. The method of item 12366 wherein the anti-infective agent is methotrexate.

12497. The method of item 12366 wherein the anti-infective agent is a podophylotoxin.

12498. The method of item 12366 wherein the anti-infective agent is etoposide.

12499. The method of item 12366 wherein the anti-infective agent is camptothecin.

12500. The method of item 12366 wherein the anti-infective agent is a hydroxyurea.

12501. The method of item 12366 wherein the anti-infective agent is a platinum complex.

12502. The method of item 12366 wherein the anti-infective agent is cisplatin.

12503. The method of item 12366 wherein the composition comprises an anti-thrombotic agent.

12504. The method of item 12366 wherein the polymer is formed from reactants comprising a naturally occurring polymer.

12505. The method of item 12366 wherein the polymer is formed from reactants comprising protein.

12506. The method of item 12366 wherein the polymer is formed from reactants comprising carbohydrate.

12507. The method of item 12366 wherein the polymer is formed from reactants comprising biodegradable polymer.

12508. The method of item 12366 wherein the polymer is formed from reactants comprising nonbiodegradable polymer.

12509. The method of item 12366 wherein the polymer is formed from reactants comprising collagen.

12510. The method of item 12366 wherein the polymer is formed from reactants comprising methylated collagen.

12511. The method of item 12366 wherein the polymer is formed from reactants comprising fibrinogen.

12512. The method of item 12366 wherein the polymer is formed from reactants comprising thrombin.

12513. The method of item 12366 wherein the polymer is formed from reactants comprising blood plasma.

12514. The method of item 12366 wherein the polymer is formed from reactants comprising calcium salt.

12515. The method of item 12366 wherein the polymer is formed from reactants comprising an antifibronolytic agent.

12516. The method of item 12366 wherein the polymer is formed from reactants comprising fibrinogen analog.

12517. The method of item 12366 wherein the polymer is formed from reactants comprising albumin.

12518. The method of item 12366 wherein the polymer is formed from reactants comprising plasminogen.

12519. The method of item 12366 wherein the polymer is formed from reactants comprising von Willebrands factor.

12520. The method of item 12366 wherein the polymer is formed from reactants comprising Factor VIII.

12521. The method of item 12366 wherein the polymer is formed from reactants comprising hypoallergenic collagen.

12522. The method of item 12366 wherein the polymer is formed from reactants comprising atelopeptidic collagen.

12523. The method of item 12366 wherein the polymer is formed from reactants comprising telopeptide collagen.

12524. The method of item 12366 wherein the polymer is formed from reactants comprising crosslinked collagen.

12525. The method of item 12366 wherein the polymer is formed from reactants comprising aprotinin.

12526. The method of item 12366 wherein the polymer is formed from reactants comprising epsilon-amino-n-caproic acid.

12527. The method of item 12366 wherein the polymer is formed from reactants comprising gelatin.

12528. The method of item 12366 wherein the polymer is formed from reactants comprising protein conjugates.

12529. The method of item 12366 wherein the polymer is formed from reactants comprising gelatin conjugates.

12530. The method of item 12366 wherein the polymer is formed from reactants comprising a synthetic polymer.

12531. The method of item 12366 wherein the polymer is formed from reactants comprising a synthetic isocyanate-containing compound.

12532. The method of item 12366 wherein the polymer is formed from reactants comprising a synthetic thiol-containing compound.

12533. The method of item 12366 wherein the polymer is formed from reactants comprising a synthetic compound containing at least two thiol groups.

12534. The method of item 12366 wherein the polymer is formed from reactants comprising a synthetic compound containing at least three thiol groups.

12535. The method of item 12366 wherein the polymer is formed from reactants comprising a synthetic compound containing at least four thiol groups.

12536. The method of item 12366 wherein the polymer is formed from reactants comprising a synthetic amino-containing compound.

12537. The method of item 12366 wherein the polymer is formed from reactants comprising a synthetic compound containing at least two amino groups.

12538. The method of item 12366 wherein the polymer is formed from reactants comprising a synthetic compound containing at least three amino groups.

12539. The method of item 12366 wherein the polymer is formed from reactants comprising a synthetic compound containing at least four amino groups.

12540. The method of item 12366 wherein the polymer is formed from reactants comprising a synthetic compound comprising a carbonyl-oxygen-succinimidyl group.

12541. The method of item 12366 wherein the polymer is formed from reactants comprising a synthetic compound comprising at least two carbonyl-oxygen-succinimidyl groups.

12542. The method of item 12366 wherein the polymer is formed from reactants comprising a synthetic compound comprising at least three carbonyl-oxygen-succinimidyl groups.

12543. The method of item 12366 wherein the polymer is formed from reactants comprising a synthetic compound comprising at least four carbonyl-oxygen-succinimidyl groups.

12544. The method of item 12366 wherein the polymer is formed from reactants comprising a synthetic polyalkylene oxide-containing compound.

12545. The method of item 12366 wherein the polymer is formed from reactants comprising a synthetic compound comprising both polyalkylene oxide and biodegradable polyester blocks.

12546. The method of item 12366 wherein the polymer is formed from reactants comprising a synthetic polyalkylene oxide-containing compound having reactive amino groups.

12547. The method of item 12366 wherein the polymer is formed from reactants comprising a synthetic polyalkylene oxide-containing compound having reactive thiol groups.

12548. The method of item 12366 wherein the polymer is formed from reactants comprising a synthetic polyalkylene oxide-containing compound having reactive carbonyl-oxygen-succinimidyl groups.

12549. The method of item 12366 wherein the polymer is formed from reactants comprising a synthetic compound comprising a biodegradable polyester block.

12550. The method of item 12366 wherein the polymer is formed from reactants comprising a synthetic polymer formed in whole or part from lactic acid or lactide.

12551. The method of item 12366 wherein the polymer is formed from reactants comprising a synthetic polymer formed in whole or part from glycolic acid or glycolide.

12552. The method of item 12366 wherein the polymer is formed from reactants comprising polylysine.

12553. The method of item 12366 wherein the polymer is formed from reactants comprising (a) protein and (b) a compound comprising a polyalkylene oxide portion.

12554. The method of item 12366 wherein the polymer is formed from reactants comprising (a) protein and (b) polylysine.

12555. The method of item 12366 wherein the polymer is formed from reactants comprising (a) protein and (b) a compound having at least four thiol groups.

12556. The method of item 12366 wherein the polymer is formed from reactants comprising (a) protein and (b) a compound having at least four amino groups.

12557. The method of item 12366 wherein the polymer is formed from reactants comprising (a) protein and (b) a compound having at least four carbonyl-oxygen-succinimide groups.

12558. The method of item 12366 wherein the polymer is formed from reactants comprising (a) protein and (b) a compound having a biodegradable region formed from reactants selected from lactic acid, lactide, glycolic acid, glycolide, and epison-caprolactone.

12559. The method of item 12366 wherein the polymer is formed from reactants comprising (a) collagen and (b) a compound comprising a polyalkylene oxide portion.

12560. The method of item 12366 wherein the polymer is formed from reactants comprising (a) collagen and (b) polylysine.

12561. The method of item 12366 wherein the polymer is formed from reactants comprising (a) collagen and (b) a compound having at least four thiol groups.

12562. The method of item 12366 wherein the polymer is formed from reactants comprising (a) collagen and (b) a compound having at least four amino groups.

12563. The method of item 12366 wherein the polymer is formed from reactants comprising (a) collagen and (b) a compound having at least four carbonyl-oxygen-succinimide groups.

12564. The method of item 12366 wherein the polymer is formed from reactants comprising (a) collagen and (b) a compound having a biodegradable region formed from reactants selected from lactic acid, lactide, glycolic acid, glycolide, and epison-caprolactone.

12565. The method of item 12366 wherein the polymer is formed from reactants comprising (a) methylated collagen and (b) a compound comprising a polyalkylene oxide portion.

12566. The method of item 12366 wherein the polymer is formed from reactants comprising (a) methylated collagen and (b) polylysine.

12567. The method of item 12366 wherein the polymer is formed from reactants comprising (a) methylated collagen and (b) a compound having at least four thiol groups.

12568. The method of item 12366 wherein the polymer is formed from reactants comprising (a) methylated collagen and (b) a compound having at least four amino groups.

12569. The method of item 12366 wherein the polymer is formed from reactants comprising (a) methylated collagen and (b) a compound having at least four carbonyl-oxygen-succinimide groups.

12570. The method of item 12366 wherein the polymer is formed from reactants comprising (a) methylated collagen and (b) a compound having a biodegradable region formed from reactants selected from lactic acid, lactide, glycolic acid, glycolide, and epison-caprolactone.

12571. The method of item 12366 wherein the polymer is formed from reactants comprising hyaluronic acid.

12572. The method of item 12366 wherein the polymer is formed from reactants comprising a hyaluronic acid derivative.

12573. The method of item 12366 wherein the polymer is formed from reactants comprising pentaerythritol poly(ethylene glycol)ether tetra-sulfhydryl of number average molecular weight between 3,000 and 30,000.

12574. The method of item 12366 wherein the polymer is formed from reactants comprising pentaerythritol poly(ethylene glycol)ether tetra-amino of number average molecular weight between 3,000 and 30,000.

12575. The method of item 12366 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

12576. The method of item 12366 wherein the composition comprises a colorant.

12577. The method of item 12366 wherein the composition is sterile.

12578. The method for implanting an electrical device of any one of items 12366-12577 wherein the vagal nerve stimulator is adapted for treating or preventing depression.

12579. The method for implanting an electrical device of any one of items 12366-12577 wherein the vagal nerve stimulator is adapted for treating or preventing anxiety.

12580. The method for implanting an electrical device of any one of items 12366-12577 wherein the vagal nerve stimulator is adapted for treating or preventing panic disorders.

12581. The method for implanting an electrical device of any one of items 12366-12577 wherein the vagal nerve stimulator is adapted for treating or preventing obsessive-compulsive disorders.

12582. The method for implanting an electrical device of any one of items 12366-12577 wherein the vagal nerve stimulator is adapted for treating or preventing post-traumatic disorders.

12583. The method for implanting an electrical device of any one of items 12366-12577 wherein the vagal nerve stimulator is adapted for treating or preventing obesity.

12584. The method for implanting an electrical device of any one of items 12366-12577 wherein the vagal nerve stimulator is adapted for treating or preventing migraine.

12585. The method for implanting an electrical device of any one of items 12366-12577 wherein the vagal nerve stimulator is adapted for treating or preventing sleep disorders.

12586. The method for implanting an electrical device of any one of items 12366-12577 wherein the vagal nerve stimulator is adapted for treating or preventing dementia.

12587. The method for implanting an electrical device of any one of items 12366-12577 wherein the vagal nerve stimulator is adapted for treating or preventing Alzheimer's disease.

12588. The method for implanting an electrical device of any one of items 12366-12577 wherein the vagal nerve stimulator is adapted for treating or preventing chronic or degenerative neurological disorders.

12589. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a sacral nerve stimulator for treating a bladder control problem.

12590. The method of item 12589 comprising: (a) infiltrating a tissue of a host where the electrical device is to be, or has been, implanted with an anti-fibrotic agent, and (b) implanting the electrical device into the host.

12591. The method of item 12589 comprising: (a) infiltrating a tissue of a host where the electrical device is to be, or has been, implanted with an anti-infective agent, and (b) implanting the electrical device into the host.

12592. The method of item 12589 comprising: (a) infiltrating a tissue of a host where the electrical device is to be, or has been, implanted with a polymer; and (b) implanting the electrical device into the host.

12593. The method of item 12589 comprising: (a) infiltrating a tissue of a host where the electrical device is to be, or has been, implanted with a composition comprising an anti-fibrotic agent and a polymer, and (b) implanting the electrical device into the host.

12594. The method of item 12589 comprising: (a) infiltrating a tissue of a host where the electrical device is to be, or has been, implanted with a composition comprising an anti-infective agent and a polymer, and (b) implanting the electrical device into the host.

12595. The method of item 12589 comprising: (a) infiltrating a tissue of a host where the electrical device is to be, or has been, implanted with a composition comprising an anti-fibrotic agent, an anti-infective agent and a polymer, and (b) implanting the electrical device into the host.

12596. The method of item 12589 wherein the agent is an adensosine A2A receptor antagonist.

12597. The method of item 12589 wherein the anti-fibrotic agent is an AKT inhibitor.

12598. The method of item 12589 wherein the anti-fibrotic agent is an alpha 2 integrin antagonist, wherein the alpha 2 integrin antagonist is Pharmaprojects No. 5754 (Merck KgaA).

12599. The method of item 12589 wherein the anti-fibrotic agent is an alpha 4 integrin antagonist.

12600. The method of item 12589 wherein the anti-fibrotic agent is an alpha 7 nicotinic receptor agonist.

12601. The method of item 12589 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1 306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

12602. The method of item 12589 wherein the anti-fibrotic agent is an apoptosis antagonist.

12603. The method of item 12589 wherein the anti-fibrotic agent is an apoptosis activator.

12604. The method of item 12589 wherein the anti-fibrotic agent is a beta 1 integrin antagonist.

12605. The method of item 12589 wherein the anti-fibrotic agent is a beta tubulin inhibitor.

12606. The method of item 12589 wherein the anti-fibrotic agent is a blocker of enzyme production in Hepatitis C.

12607. The method of item 12589 wherein the anti-fibrotic agent is a Bruton's tyrosine kinase inhibitor.

12608. The method of item 12589 wherein the anti-fibrotic agent is a calcineurin inhibitor.

12609. The method of item 12589 wherein the anti-fibrotic agent is a caspase 3 inhibitor.

12610. The method of item 12589 wherein the anti-fibrotic agent is a CC chemokine receptor antagonist.

12611. The method of item 12589 wherein the anti-fibrotic agent is a cell cycle inhibitor selected from the group consisting of SNS-595 (Sunesis), synthadotin, KRX-0403, and an analogue or derivative thereof.

12612. The method of item 12589 wherein the anti-fibrotic agent is a cathepsin B inhibitor.

12613. The method of item 12589 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

12614. The method of item 12589 wherein the anti-fibrotic agent is a cathepsin L inhibitor.

12615. The method of item 12589 wherein the anti-fibrotic agent is a CD40 antagonist.

12616. The method of item 12589 wherein the anti-fibrotic agent is a chemokine receptor agonist.

12617. The method of item 12589 wherein the anti-fibrotic agent is a chymase inhibitor.

12618. The method of item 12589 wherein the anti-fibrotic agent is a collagenase antagonist.

12619. The method of item 12589 wherein the anti-fibrotic agent is a CXCR antagonist.

12620. The method of item 12589 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

12621. The method of item 12589 wherein the anti-fibrotic agent is a cyclooxygenase 1 inhibitor.

12622. The method of item 12589 wherein the anti-fibrotic agent is a DHFR inhibitor.

12623. The method of item 12589 wherein the anti-fibrotic agent is a dual integrin inhibitor.

12624. The method of item 12589 wherein the anti-fibrotic agent is an elastase inhibitor.

12625. The method of item 12589 wherein the anti-fibrotic agent is an elongation factor-1 alpha inhibitor.

12626. The method of item 12589 wherein the anti-fibrotic agent is an endothelial growth factor antagonist.

12627. The method of item 12589 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

12628. The method of item 12589 wherein the anti-fibrotic agent is an endotoxin antagonist.

12629. The method of item 12589 wherein the anti-fibrotic agent is an epothilone and tubulin binder.

12630. The method of item 12589 wherein the anti-fibrotic agent is an estrogen receptor antagonist.

12631. The method of item 12589 wherein the anti-fibrotic agent is an FGF inhibitor.

12632. The method of item 12589 wherein the anti-fibrotic agent is a farnexyl transferase inhibitor.

12633. The method of item 12589 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

12634. The method of item 12589 wherein the anti-fibrotic agent is an FLT-3 kinase inhibitor.

12635. The method of item 12589 wherein the anti-fibrotic agent is an FGF receptor kinase inhibitor.

12636. The method of item 12589 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

12637. The method of item 12589 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

12638. The method of item 12589 wherein the anti-fibrotic agent is a histone deacetylase inhibitor.

12639. The method of item 12589 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

12640. The method of item 12589 wherein the anti-fibrotic agent is an ICAM inhibitor.

12641. The method of item 12589 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

12642. The method of item 12589 wherein the anti-fibrotic agent is an IL-2 inhibitor.

12643. The method of item 12589 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

12644. The method of item 12589 wherein the anti-fibrotic agent is an IMPDH (inosine monophosphate).

12645. The method of item 12589 wherein the anti-fibrotic agent is an integrin antagonist.

12646. The method of item 12589 wherein the anti-fibrotic agent is an interleukin antagonist.

12647. The method of item 12589 wherein the anti-fibrotic agent is an inhibitor of type III receptor tyrosine kinase.

12648. The method of item 12589 wherein the anti-fibrotic agent is an irreversible inhibitor of enzyme methionine aminopeptidase type 2.

12649. The method of item 12589 wherein the anti-fibrotic agent is an isozyme selective delta protein kinase C inhibitor.

12650. The method of item 12589 wherein the anti-fibrotic agent a JAK3 enzyme inhibitor.

12651. The method of item 12589 wherein the anti-fibrotic agent is a JNK inhibitor.

12652. The method of item 12589 wherein the anti-fibrotic agent is a kinase inhibitor.

12653. The method of item 12589 wherein the anti-fibrotic agent is kinesin antagonist.

12654. The method of item 12589 wherein the anti-fibrotic agent is a kinesin antagonist.

12655. The method of item 12589 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

12656. The method of item 12589 wherein the anti-fibrotic agent is an MAP kinase inhibitor.

12657. The method of item 12589 wherein the anti-fibrotic agent is a matrix metalloproteinase inhibitor.

12658. The method of item 12589 wherein the anti-fibrotic agent is an MCP-CCR2 inhibitor.

12659. The method of item 12589 wherein the anti-fibrotic agent is an mTOR inhibitor.

12660. The method of item 12589 wherein the anti-fibrotic agent is an mTOR kinase inhibitor.

12661. The method of item 12589 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

12662. The method of item 12589 wherein the anti-fibrotic agent is an MIF inhibitor.

12663. The method of item 12589 wherein the anti-fibrotic agent is an MMP inhibitor.

12664. The method of item 12589 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

12665. The method of item 12589 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

12666. The method of item 12589 wherein the anti-fibrotic agent is a nitric oxide agonist.

12667. The method of item 12589 wherein the anti-fibrotic agent is an ornithine decarboxylase inhibitor.

12668. The method of item 12589 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

12669. The method of item 12589 wherein the anti-fibrotic agent is a palmitoyl-protein thioesterase inhibitor.

12670. The method of item 12589 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

12671. The method of item 12589 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

12672. The method of item 12589 wherein the anti-fibrotic agent is a phosphatase inhibitor.

12673. The method of item 12589 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

12674. The method of item 12589 wherein the anti-fibrotic agent is a PKC inhibitor.

12675. The method of item 12589 wherein the anti-fibrotic agent is a platelet activating factor antagonist.

12676. The method of item 12589 wherein the anti-fibrotic agent is a platelet-derived growth factor receptor kinase inhibitor.

12677. The method of item 12589 wherein the anti-fibrotic agent is a prolyl hydroxylase inhibitor.

12678. The method of item 12589 wherein the anti-fibrotic agent is a polymorphonuclear neutrophil inhibitor.

12679. The method of item 12589 wherein the anti-fibrotic agent is a protein kinase B inhibitor.

12680. The method of item 12589 wherein the anti-fibrotic agent is a protein kinase C stimulant.

12681. The method of item 12589 wherein the anti-fibrotic agent is a purine nucleoside analogue.

12682. The method of item 12589 wherein the anti-fibrotic agent is a purinoreceptor P2X antagonist.

12683. The method of item 12589 wherein the anti-fibrotic agent is a Raf kinase inhibitor.

12684. The method of item 12589 wherein the anti-fibrotic agent is a reversible inhibitor of ErbB1 and ErbB2.

12685. The method of item 12589 wherein the anti-fibrotic agent is a ribonucleoside triphosphate reductase inhibitor.

12686. The method of item 12589 wherein the anti-fibrotic agent is an SDF-1 antagonist.

12687. The method of item 12589 wherein the anti-fibrotic agent is a sheddase inhibitor.

12688. The method of item 12589 wherein the anti-fibrotic agent is an SRC inhibitor.

12689. The method of item 12589 wherein the anti-fibrotic agent is a stromelysin inhibitor.

12690. The method of item 12589 wherein the anti-fibrotic agent is an Syk kinase inhibitor.

12691. The method of item 12589 wherein the anti-fibrotic agent is a telomerase inhibitor.

12692. The method of item 12589 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

12693. The method of item 12589 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

12694. The method of item 12589 wherein the anti-fibrotic agent is a Toll receptor inhibitor.

12695. The method of item 12589 wherein the anti-fibrotic agent is a tubulin antagonist.

12696. The method of item 12589 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

12697. The method of item 12589 wherein the anti-fibrotic agent is a VEGF inhibitor.

12698. The method of item 12589 wherein the anti-fibrotic agent is a vitamin D receptor agonist.

12699. The method of item 12589 wherein the anti-fibrotic agent is ZD-6474 (an angiogenesis inhibitor).

12700. The method of item 12589 wherein the anti-fibrotic agent is AP-23573 (an mTOR inhibitor).

12701. The method of item 12589 wherein the anti-fibrotic agent is synthadotin (a tubulin antagonist).

12702. The method of item 12589 wherein the anti-fibrotic agent is S-0885 (a collagenase inhibitor).

12703. The method of item 12589 wherein the anti-fibrotic agent is aplidine (an elongation factor-1 alpha inhibitor).

12704. The method of item 12589 wherein the anti-fibrotic agent is ixabepilone (an epithilone).

12705. The method of item 12589 wherein the anti-fibrotic agent is IDN-5390 (an angiogenesis inhibitor).

12706. The method of item 12589 wherein the anti-fibrotic agent is SB-2723005 (an angiogenesis inhibitor).

12707. The method of item 12589 wherein the anti-fibrotic agent is ABT-518 (an angiogenesis inhibitor).

12708. The method of item 12589 wherein the anti-fibrotic agent is combretastatin (an angiogenesis inhibitor).

12709. The method of item 12589 wherein the anti-fibrotic agent is anecortave acetate (an angiogenesis inhibitor).

12710. The method of item 12589 wherein the anti-fibrotic agent is SB-715992 (a kinesin antagonist).

12711. The method of item 12589 wherein the anti-fibrotic agent is temsirolimus (an mTOR inhibitor).

12712. The method of item 12589 wherein the anti-fibrotic agent is adalimumab (a TNFα antagonist).

12713. The method of item 12589 wherein the anti-infective agent is an anthracycline.

12714. The method of item 12589 wherein the anti-infective agent is doxorubicin.

12715. The method of item 12589 wherein the anti-infective agent is mitoxantrone.

12716. The method of item 12589 wherein the anti-infective agent is a fluoropyrimidine.

12717. The method of item 12589 wherein the anti-infective agent is 5-fluorouracil (5-FU).

12718. The method of item 12589 wherein the anti-infective agent is a folic acid antagonist.

12719. The method of item 12589 wherein the anti-infective agent is methotrexate.

12720. The method of item 12589 wherein the anti-infective agent is a podophylotoxin.

12721. The method of item 12589 wherein the anti-infective agent is etoposide.

12722. The method of item 12589 wherein the anti-infective agent is camptothecin.

12723. The method of item 12589 wherein the anti-infective agent is a hydroxyurea.

12724. The method of item 12589 wherein the anti-infective agent is a platinum complex.

12725. The method of item 12589 wherein the anti-infective agent is cisplatin.

12726. The method of item 12589 wherein the composition comprises an anti-thrombotic agent.

12727. The method of item 12589 wherein the polymer is formed from reactants comprising a naturally occurring polymer.

12728. The method of item 12589 wherein the polymer is formed from reactants comprising protein.

12729. The method of item 12589 wherein the polymer is formed from reactants comprising carbohydrate.

12730. The method of item 12589 wherein the polymer is formed from reactants comprising biodegradable polymer.

12731. The method of item 12589 wherein the polymer is formed from reactants comprising nonbiodegradable polymer.

12732. The method of item 12589 wherein the polymer is formed from reactants comprising collagen.

12733. The method of item 12589 wherein the polymer is formed from reactants comprising methylated collagen.

12734. The method of item 12589 wherein the polymer is formed from reactants comprising fibrinogen.

12735. The method of item 12589 wherein the polymer is formed from reactants comprising thrombin.

12736. The method of item 12589 wherein the polymer is formed from reactants comprising blood plasma.

12737. The method of item 12589 wherein the polymer is formed from reactants comprising calcium salt.

12738. The method of item 12589 wherein the polymer is formed from reactants comprising an antifibronolytic agent.

12739. The method of item 12589 wherein the polymer is formed from reactants comprising fibrinogen analog.

12740. The method of item 12589 wherein the polymer is formed from reactants comprising albumin.

12741. The method of item 12589 wherein the polymer is formed from reactants comprising plasminogen.

12742. The method of item 12589 wherein the polymer is formed from reactants comprising von Willebrands factor.

12743. The method of item 12589 wherein the polymer is formed from reactants comprising Factor VIII.

12744. The method of item 12589 wherein the polymer is formed from reactants comprising hypoallergenic collagen.

12745. The method of item 12589 wherein the polymer is formed from reactants comprising atelopeptidic collagen.

12746. The method of item 12589 wherein the polymer is formed from reactants comprising telopeptide collagen.

12747. The method of item 12589 wherein the polymer is formed from reactants comprising crosslinked collagen.

12748. The method of item 12589 wherein the polymer is formed from reactants comprising aprotinin.

12749. The method of item 12589 wherein the polymer is formed from reactants comprising epsilon-amino-n-caproic acid.

12750. The method of item 12589 wherein the polymer is formed from reactants comprising gelatin.

12751. The method of item 12589 wherein the polymer is formed from reactants comprising protein conjugates.

12752. The method of item 12589 wherein the polymer is formed from reactants comprising gelatin conjugates.

12753. The method of item 12589 wherein the polymer is formed from reactants comprising a synthetic polymer.

12754. The method of item 12589 wherein the polymer is formed from reactants comprising a synthetic isocyanate-containing compound.

12755. The method of item 12589 wherein the polymer is formed from reactants comprising a synthetic thiol-containing compound.

12756. The method of item 12589 wherein the polymer is formed from reactants comprising a synthetic compound containing at least two thiol groups.

12757. The method of item 12589 wherein the polymer is formed from reactants comprising a synthetic compound containing at least three thiol groups.

12758. The method of item 12589 wherein the polymer is formed from reactants comprising a synthetic compound containing at least four thiol groups.

12759. The method of item 12589 wherein the polymer is formed from reactants comprising a synthetic amino-containing compound.

12760. The method of item 12589 wherein the polymer is formed from reactants comprising a synthetic compound containing at least two amino groups.

12761. The method of item 12589 wherein the polymer is formed from reactants comprising a synthetic compound containing at least three amino groups.

12762. The method of item 12589 wherein the polymer is formed from reactants comprising a synthetic compound containing at least four amino groups.

12763. The method of item 12589 wherein the polymer is formed from reactants comprising a synthetic compound comprising a carbonyl-oxygen-succinimidyl group.

12764. The method of item 12589 wherein the polymer is formed from reactants comprising a synthetic compound comprising at least two carbonyl-oxygen-succinimidyl groups.

12765. The method of item 12589 wherein the polymer is formed from reactants comprising a synthetic compound comprising at least three carbonyl-oxygen-succinimidyl groups.

12766. The method of item 12589 wherein the polymer is formed from reactants comprising a synthetic compound comprising at least four carbonyl-oxygen-succinimidyl groups.

12767. The method of item 12589 wherein the polymer is formed from reactants comprising a synthetic polyalkylene oxide-containing compound.

12768. The method of item 12589 wherein the polymer is formed from reactants comprising a synthetic compound comprising both polyalkylene oxide and biodegradable polyester blocks.

12769. The method of item 12589 wherein the polymer is formed from reactants comprising a synthetic polyalkylene oxide-containing compound having reactive amino groups.

12770. The method of item 12589 wherein the polymer is formed from reactants comprising a synthetic polyalkylene oxide-containing compound having reactive thiol groups.

12771. The method of item 12589 wherein the polymer is formed from reactants comprising a synthetic polyalkylene oxide-containing compound having reactive carbonyl-oxygen-succinimidyl groups.

12772. The method of item 12589 wherein the polymer is formed from reactants comprising a synthetic compound comprising a biodegradable polyester block.

12773. The method of item 12589 wherein the polymer is formed from reactants comprising a synthetic polymer formed in whole or part from lactic acid or lactide.

12774. The method of item 12589 wherein the polymer is formed from reactants comprising a synthetic polymer formed in whole or part from glycolic acid or glycolide.

12775. The method of item 12589 wherein the polymer is formed from reactants comprising polylysine.

12776. The method of item 12589 wherein the polymer is formed from reactants comprising (a) protein and (b) a compound comprising a polyalkylene oxide portion.

12777. The method of item 12589 wherein the polymer is formed from reactants comprising (a) protein and (b) polylysine.

12778. The method of item 12589 wherein the polymer is formed from reactants comprising (a) protein and (b) a compound having at least four thiol groups.

12779. The method of item 12589 wherein the polymer is formed from reactants comprising (a) protein and (b) a compound having at least four amino groups.

12780. The method of item 12589 wherein the polymer is formed from reactants comprising (a) protein and (b) a compound having at least four carbonyl-oxygen-succinimide groups.

12781. The method of item 12589 wherein the polymer is formed from reactants comprising (a) protein and (b) a compound having a biodegradable region formed from reactants selected from lactic acid, lactide, glycolic acid, glycolide, and epison-caprolactone.

12782. The method of item 12589 wherein the polymer is formed from reactants comprising (a) collagen and (b) a compound comprising a polyalkylene oxide portion.

12783. The method of item 12589 wherein the polymer is formed from reactants comprising (a) collagen and (b) polylysine.

12784. The method of item 12589 wherein the polymer is formed from reactants comprising (a) collagen and (b) a compound having at least four thiol groups.

12785. The method of item 12589 wherein the polymer is formed from reactants comprising (a) collagen and (b) a compound having at least four amino groups.

12786. The method of item 12589 wherein the polymer is formed from reactants comprising (a) collagen and (b) a compound having at least four carbonyl-oxygen-succinimide groups.

12787. The method of item 12589 wherein the polymer is formed from reactants comprising (a) collagen and (b) a compound having a biodegradable region formed from reactants selected from lactic acid, lactide, glycolic acid, glycolide, and epison-caprolactone.

12788. The method of item 12589 wherein the polymer is formed from reactants comprising (a) methylated collagen and (b) a compound comprising a polyalkylene oxide portion.

12789. The method of item 12589 wherein the polymer is formed from reactants comprising (a) methylated collagen and (b) polylysine.

12790. The method of item 12589 wherein the polymer is formed from reactants comprising (a) methylated collagen and (b) a compound having at least four thiol groups.

12791. The method of item 12589 wherein the polymer is formed from reactants comprising (a) methylated collagen and (b) a compound having at least four amino groups.

12792. The method of item 12589 wherein the polymer is formed from reactants comprising (a) methylated collagen and (b) a compound having at least four carbonyl-oxygen-succinimide groups.

12793. The method of item 12589 wherein the polymer is formed from reactants comprising (a) methylated collagen and (b) a compound having a biodegradable region formed from reactants selected from lactic acid, lactide, glycolic acid, glycolide, and epison-caprolactone.

12794. The method of item 12589 wherein the polymer is formed from reactants comprising hyaluronic acid.

12795. The method of item 12589 wherein the polymer is formed from reactants comprising a hyaluronic acid derivative.

12796. The method of item 12589 wherein the polymer is formed from reactants comprising pentaerythritol poly(ethylene glycol)ether tetra-sulfhydryl of number average molecular weight between 3,000 and 30,000.

12797. The method of item 12589 wherein the polymer is formed from reactants comprising pentaerythritol poly(ethylene glycol)ether tetra-amino of number average molecular weight between 3,000 and 30,000.

12798. The method of item 12589 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

12799. The method of item 12589 wherein the composition comprises a colorant.

12800. The method of item 12589 wherein the composition is sterile.

12801. The method for implanting an electrical device of any one of items 12589-12800 wherein the sacral nerve stimulator is adapted for treating or preventing urge incontinence.

12802. The method for implanting an electrical device of any one of items 12589-12800 wherein the sacral nerve stimulator is adapted for treating or preventing nonobstructive urinary retention.

12803. The method for implanting an electrical device of any one of items 12589-12800 wherein the sacral nerve stimulator is adapted for treating or preventing urgency frequency.

12804. The method for implanting an electrical device of any one of items 12589-12800 wherein the sacral nerve stimulator is an intramuscular electrical stimulator.

12805. The method for implanting an electrical device of any one of items 12589-12800 wherein the sacral nerve stimulator is a leadless, tubular-shaped microstimulator.

12806. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a gastric nerve stimulator for treating a gastrointestinal disorder.

12807. The method of item 12806 comprising: (a) infiltrating a tissue of a host where the electrical device is to be, or has been, implanted with an anti-fibrotic agent, and (b) implanting the electrical device into the host.

12808. The method of item 12806 comprising: (a) infiltrating a tissue of a host where the electrical device is to be, or has been, implanted with an anti-infective agent, and (b) implanting the electrical device into the host.

12809. The method of item 12806 comprising: (a) infiltrating a tissue of a host where the electrical device is to be, or has been, implanted with a polymer; and (b) implanting the electrical device into the host.

12810. The method of item 12806 comprising: (a) infiltrating a tissue of a host where the electrical device is to be, or has been, implanted with a composition comprising an anti-fibrotic agent and a polymer, and (b) implanting the electrical device into the host.

12811. The method of item 12806 comprising: (a) infiltrating a tissue of a host where the electrical device is to be, or has been, implanted with a composition comprising an anti-infective agent and a polymer, and (b) implanting the electrical device into the host.

12812. The method of item 12806 comprising: (a) infiltrating a tissue of a host where the electrical device is to be, or has been, implanted with a composition comprising an anti-fibrotic agent, an anti-infective agent and a polymer, and (b) implanting the electrical device into the host.

12813. The method of item 12806 wherein the agent is an adensosine A2A receptor antagonist.

12814. The method of item 12806 wherein the anti-fibrotic agent is an AKT inhibitor.

12815. The method of item 12806 wherein the anti-fibrotic agent is an alpha 2 integrin antagonist, wherein the alpha 2 integrin antagonist is Pharmaprojects No. 5754 (Merck KgaA).

12816. The method of item 12806 wherein the anti-fibrotic agent is an alpha 4 integrin antagonist.

12817. The method of item 12806 wherein the anti-fibrotic agent is an alpha 7 nicotinic receptor agonist.

12818. The method of item 12806 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1 alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

12819. The method of item 12806 wherein the anti-fibrotic agent is an apoptosis antagonist.

12820. The method of item 12806 wherein the anti-fibrotic agent is an apoptosis activator.

12821. The method of item 12806 wherein the anti-fibrotic agent is a beta 1 integrin antagonist.

12822. The method of item 12806 wherein the anti-fibrotic agent is a beta tubulin inhibitor.

12823. The method of item 12806 wherein the anti-fibrotic agent is a blocker of enzyme production in Hepatitis C.

12824. The method of item 12806 wherein the anti-fibrotic agent is a Bruton's tyrosine kinase inhibitor.

12825. The method of item 12806 wherein the anti-fibrotic agent is a calcineurin inhibitor.

12826. The method of item 12806 wherein the anti-fibrotic agent is a caspase 3 inhibitor.

12827. The method of item 12806 wherein the anti-fibrotic agent is a CC chemokine receptor antagonist.

12828. The method of item 12806 wherein the anti-fibrotic agent is a cell cycle inhibitor selected from the group consisting of SNS-595 (Sunesis), synthadotin, KRX-0403, and an analogue or derivative thereof.

12829. The method of item 12806 wherein the anti-fibrotic agent is a cathepsin B inhibitor.

12830. The method of item 12806 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

12831. The method of item 12806 wherein the anti-fibrotic agent is a cathepsin L inhibitor.

12832. The method of item 12806 wherein the anti-fibrotic agent is a CD40 antagonist.

12833. The method of item 12806 wherein the anti-fibrotic agent is a chemokine receptor agonist.

12834. The method of item 12806 wherein the anti-fibrotic agent is a chymase inhibitor.

12835. The method of item 12806 wherein the anti-fibrotic agent is a collagenase antagonist.

12836. The method of item 12806 wherein the anti-fibrotic agent is a CXCR antagonist.

12837. The method of item 12806 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

12838. The method of item 12806 wherein the anti-fibrotic agent is a cyclooxygenase 1 inhibitor.

12839. The method of item 12806 wherein the anti-fibrotic agent is a DHFR inhibitor.

12840. The method of item 12806 wherein the anti-fibrotic agent is a dual integrin inhibitor.

12841. The method of item 12806 wherein the anti-fibrotic agent is an elastase inhibitor.

12842. The method of item 12806 wherein the anti-fibrotic agent is an elongation factor-1 alpha inhibitor.

12843. The method of item 12806 wherein the anti-fibrotic agent is an endothelial growth factor antagonist.

12844. The method of item 12806 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

12845. The method of item 12806 wherein the anti-fibrotic agent is an endotoxin antagonist.

12846. The method of item 12806 wherein the anti-fibrotic agent is an epothilone and tubulin binder.

12847. The method of item 12806 wherein the anti-fibrotic agent is an estrogen receptor antagonist.

12848. The method of item 12806 wherein the anti-fibrotic agent is an FGF inhibitor.

12849. The method of item 12806 wherein the anti-fibrotic agent is a farnexyl transferase inhibitor.

12850. The method of item 12806 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

12851. The method of item 12806 wherein the anti-fibrotic agent is an FLT-3 kinase inhibitor.

12852. The method of item 12806 wherein the anti-fibrotic agent is an FGF receptor kinase inhibitor.

12853. The method of item 12806 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

12854. The method of item 12806 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

12855. The method of item 12806 wherein the anti-fibrotic agent is a histone deacetylase inhibitor.

12856. The method of item 12806 wherein the anti-fibrotic agent is an HMGCoA reductase inhibitor selected from the group consisting of an atherosclerosis therapeutic from Lipid Sciences, ATI-16000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

12857. The method of item 12806 wherein the anti-fibrotic agent is an ICAM inhibitor.

12858. The method of item 12806 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

12859. The method of item 12806 wherein the anti-fibrotic agent is an IL-2 inhibitor.

12860. The method of item 12806 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

12861. The method of item 12806 wherein the anti-fibrotic agent is an IMPDH (inosine monophosphate).

12862. The method of item 12806 wherein the anti-fibrotic agent is an integrin antagonist.

12863. The method of item 12806 wherein the anti-fibrotic agent is an interleukin antagonist.

12864. The method of item 12806 wherein the anti-fibrotic agent is an inhibitor of type III receptor tyrosine kinase.

12865. The method of item 12806 wherein the anti-fibrotic agent is an irreversible inhibitor of enzyme methionine aminopeptidase type 2.

12866. The method of item 12806 wherein the anti-fibrotic agent is an isozyme selective delta protein kinase C inhibitor.

12867. The method of item 12806 wherein the anti-fibrotic agent a JAK3 enzyme inhibitor.

12868. The method of item 12806 wherein the anti-fibrotic agent is a JNK inhibitor.

12869. The method of item 12806 wherein the anti-fibrotic agent is a kinase inhibitor.

12870. The method of item 12806 wherein the anti-fibrotic agent is kinesin antagonist.

12871. The method of item 12806 wherein the anti-fibrotic agent is a kinesin antagonist.

12872. The method of item 12806 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-15106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

12873. The method of item 12806 wherein the anti-fibrotic agent is an MAP kinase inhibitor.

12874. The method of item 12806 wherein the anti-fibrotic agent is a matrix metalloproteinase inhibitor.

12875. The method of item 12806 wherein the anti-fibrotic agent is an MCP-CCR2 inhibitor.

12876. The method of item 12806 wherein the anti-fibrotic agent is an mTOR inhibitor.

12877. The method of item 12806 wherein the anti-fibrotic agent is an mTOR kinase inhibitor.

12878. The method of item 12806 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-62-4), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

12879. The method of item 12806 wherein the anti-fibrotic agent is an MIF inhibitor.

12880. The method of item 12806 wherein the anti-fibrotic agent is an MMP inhibitor.

12881. The method of item 12806 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

12882. The method of item 12806 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-494) (Encore Pharmaceuticals), and an analogue or derivative thereof.

12883. The method of item 12806 wherein the anti-fibrotic agent is a nitric oxide agonist.

12884. The method of item 12806 wherein the anti-fibrotic agent is an ornithine decarboxylase inhibitor.

12885. The method of item 12806 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

12886. The method of item 12806 wherein the anti-fibrotic agent is a palmitoyl-protein thioesterase inhibitor.

12887. The method of item 12806 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

12888. The method of item 12806 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

12889. The method of item 12806 wherein the anti-fibrotic agent is a phosphatase inhibitor.

12890. The method of item 12806 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

12891. The method of item 12806 wherein the anti-fibrotic agent is a PKC inhibitor.

12892. The method of item 12806 wherein the anti-fibrotic agent is a platelet activating factor antagonist.

12893. The method of item 12806 wherein the anti-fibrotic agent is a platelet-derived growth factor receptor kinase inhibitor.

12894. The method of item 12806 wherein the anti-fibrotic agent is a prolyl hydroxylase inhibitor.

12895. The method of item 12806 wherein the anti-fibrotic agent is a polymorphonuclear neutrophil inhibitor.

12896. The method of item 12806 wherein the anti-fibrotic agent is a protein kinase B inhibitor.

12897. The method of item 12806 wherein the anti-fibrotic agent is a protein kinase C stimulant.

12898. The method of item 12806 wherein the anti-fibrotic agent is a purine nucleoside analogue.

12899. The method of item 12806 wherein the anti-fibrotic agent is a purinoreceptor P2X antagonist.

12900. The method of item 12806 wherein the anti-fibrotic agent is a Raf kinase inhibitor.

12901. The method of item 12806 wherein the anti-fibrotic agent is a reversible inhibitor of ErbB1 and ErbB2.

12902. The method of item 12806 wherein the anti-fibrotic agent is a ribonucleoside triphosphate reductase inhibitor.

12903. The method of item 12806 wherein the anti-fibrotic agent is an SDF-1 antagonist.

12904. The method of item 12806 wherein the anti-fibrotic agent is a sheddase inhibitor.

12905. The method of item 12806 wherein the anti-fibrotic agent is an SRC inhibitor.

12906. The method of item 12806 wherein the anti-fibrotic agent is a stromelysin inhibitor.

12907. The method of item 12806 wherein the anti-fibrotic agent is an Syk kinase inhibitor.

12908. The method of item 12806 wherein the anti-fibrotic agent is a telomerase inhibitor.

12909. The method of item 12806 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), man nose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

12910. The method of item 12806 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Celizome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

12911. The method of item 12806 wherein the anti-fibrotic agent is a Toll receptor inhibitor.

12912. The method of item 12806 wherein the anti-fibrotic agent is a tubulin antagonist.

12913. The method of item 12806 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

12914. The method of item 12806 wherein the anti-fibrotic agent is a VEGF inhibitor.

12915. The method of item 12806 wherein the anti-fibrotic agent is a vitamin D receptor agonist.

12916. The method of item 12806 wherein the anti-fibrotic agent is ZD-6474 (an angiogenesis inhibitor).

12917. The method of item 12806 wherein the anti-fibrotic agent is AP-23573 (an mTOR inhibitor).

12918. The method of item 12806 wherein the anti-fibrotic agent is synthadotin (a tubulin antagonist).

12919. The method of item 12806 wherein the anti-fibrotic agent is S-0885 (a collagenase inhibitor).

12920. The method of item 12806 wherein the anti-fibrotic agent is aplidine (an elongation factor-1 alpha inhibitor).

12921. The method of item 12806 wherein the anti-fibrotic agent is ixabepilone (an epithilone).

12922. The method of item 12806 wherein the anti-fibrotic agent is IDN-5390 (an angiogenesis inhibitor).

12923. The method of item 12806 wherein the anti-fibrotic agent is SB-2723005 (an angiogenesis inhibitor).

12924. The method of item 12806 wherein the anti-fibrotic agent is ABT-518 (an angiogenesis inhibitor).

12925. The method of item 12806 wherein the anti-fibrotic agent is combretastatin (an angiogenesis inhibitor).

12926. The method of item 12806 wherein the anti-fibrotic agent is anecortave acetate (an angiogenesis inhibitor).

12927. The method of item 12806 wherein the anti-fibrotic agent is SB-715992 (a kinesin antagonist).

12928. The method of item 12806 wherein the anti-fibrotic agent is temsirolimus (an mTOR inhibitor).

12929. The method of item 12806 wherein the anti-fibrotic agent is adalimumab (a TNFα antagonist).

12930. The method of item 12806 wherein the anti-infective agent is an anthracycline.

12931. The method of item 12806 wherein the anti-infective agent is doxorubicin.

12932. The method of item 12806 wherein the anti-infective agent is mitoxantrone.

12933. The method of item 12806 wherein the anti-infective agent is a fluoropyrimidine.

12934. The method of item 12806 wherein the anti-infective agent is 5-fluorouracil (5-FU).

12935. The method of item 12806 wherein the anti-infective agent is a folic acid antagonist.

12936. The method of item 12806 wherein the anti-infective agent is methotrexate.

12937. The method of item 12806 wherein the anti-infective agent is a podophylotoxin.

12938. The method of item 12806 wherein the anti-infective agent is etoposide.

12939. The method of item 12806 wherein the anti-infective agent is camptothecin.

12940. The method of item 12806 wherein the anti-infective agent is a hydroxyurea.

12941. The method of item 12806 wherein the anti-infective agent is a platinum complex.

12942. The method of item 12806 wherein the anti-infective agent is cisplatin.

12943. The method of item 12806 wherein the composition comprises an anti-thrombotic agent.

12944. The method of item 12806 wherein the polymer is formed from reactants comprising a naturally occurring polymer.

12945. The method of item 12806 wherein the polymer is formed from reactants comprising protein.

12946. The method of item 12806 wherein the polymer is formed from reactants comprising carbohydrate.

12947. The method of item 12806 wherein the polymer is formed from reactants comprising biodegradable polymer.

12948. The method of item 12806 wherein the polymer is formed from reactants comprising nonbiodegradable polymer.

12949. The method of item 12806 wherein the polymer is formed from reactants comprising collagen.

12950. The method of item 12806 wherein the polymer is formed from reactants comprising methylated collagen.

12951. The method of item 12806 wherein the polymer is formed from reactants comprising fibrinogen.

12952. The method of item 12806 wherein the polymer is formed from reactants comprising thrombin.

12953. The method of item 12806 wherein the polymer is formed from reactants comprising blood plasma.

12954. The method of item 12806 wherein the polymer is formed from reactants comprising calcium salt.

12955. The method of item 12806 wherein the polymer is formed from reactants comprising an antifibronolytic agent.

12956. The method of item 12806 wherein the polymer is formed from reactants comprising fibrinogen analog.

12957. The method of item 12806 wherein the polymer is formed from reactants comprising albumin.

12958. The method of item 12806 wherein the polymer is formed from reactants comprising plasminogen.

12959. The method of item 12806 wherein the polymer is formed from reactants comprising von Willebrands factor.

12960. The method of item 12806 wherein the polymer is formed from reactants comprising Factor VIII.

12961. The method of item 12806 wherein the polymer is formed from reactants comprising hypoallergenic collagen.

12962. The method of item 12806 wherein the polymer is formed from reactants comprising atelopeptidic collagen.

12963. The method of item 12806 wherein the polymer is formed from reactants comprising telopeptide collagen.

12964. The method of item 12806 wherein the polymer is formed from reactants comprising crosslinked collagen.

12965. The method of item 12806 wherein the polymer is formed from reactants comprising aprotinin.

12966. The method of item 12806 wherein the polymer is formed from reactants comprising epsilon-amino-n-caproic acid.

12967. The method of item 12806 wherein the polymer is formed from reactants comprising gelatin.

12968. The method of item 12806 wherein the polymer is formed from reactants comprising protein conjugates.

12969. The method of item 12806 wherein the polymer is formed from reactants comprising gelatin conjugates.

12970. The method of item 12806 wherein the polymer is formed from reactants comprising a synthetic polymer.

12971. The method of item 12806 wherein the polymer is formed from reactants comprising a synthetic isocyanate-containing compound.

12972. The method of item 12806 wherein the polymer is formed from reactants comprising a synthetic thiol-containing compound.

12973. The method of item 12806 wherein the polymer is formed from reactants comprising a synthetic compound containing at least two thiol groups.

12974. The method of item 12806 wherein the polymer is formed from reactants comprising a synthetic compound containing at least three thiol groups.

12975. The method of item 12806 wherein the polymer is formed from reactants comprising a synthetic compound containing at least four thiol groups.

12976. The method of item 12806 wherein the polymer is formed from reactants comprising a synthetic amino-containing compound.

12977. The method of item 12806 wherein the polymer is formed from reactants comprising a synthetic compound containing at least two amino groups.

12978. The method of item 12806 wherein the polymer is formed from reactants comprising a synthetic compound containing at least three amino groups.

12979. The method of item 12806 wherein the polymer is formed from reactants comprising a synthetic compound containing at least four amino groups.

12980. The method of item 12806 wherein the polymer is formed from reactants comprising a synthetic compound comprising a carbonyl-oxygen-succinimidyl group.

12981. The method of item 12806 wherein the polymer is formed from reactants comprising a synthetic compound comprising at least two carbonyl-oxygen-succinimidyl groups.

12982. The method of item 12806 wherein the polymer is formed from reactants comprising a synthetic compound comprising at least three carbonyl-oxygen-succinimidyl groups.

12983. The method of item 12806 wherein the polymer is formed from reactants comprising a synthetic compound comprising at least four carbonyl-oxygen-succinimidyl groups.

12984. The method of item 12806 wherein the polymer is formed from reactants comprising a synthetic polyalkylene oxide-containing compound.

12985. The method of item 12806 wherein the polymer is formed from reactants comprising a synthetic compound comprising both polyalkylene oxide and biodegradable polyester blocks.

12986. The method of item 12806 wherein the polymer is formed from reactants comprising a synthetic polyalkylene oxide-containing compound having reactive amino groups.

12987. The method of item 12806 wherein the polymer is formed from reactants comprising a synthetic polyalkylene oxide-containing compound having reactive thiol groups.

12988. The method of item 12806 wherein the polymer is formed from reactants comprising a synthetic polyalkylene oxide-containing compound having reactive carbonyl-oxygen-succinimidyl groups.

12989. The method of item 12806 wherein the polymer is formed from reactants comprising a synthetic compound comprising a biodegradable polyester block.

12990. The method of item 12806 wherein the polymer is formed from reactants comprising a synthetic polymer formed in whole or part from lactic acid or lactide.

12991. The method of item 12806 wherein the polymer is formed from reactants comprising a synthetic polymer formed in whole or part from glycolic acid or glycolide.

12992. The method of item 12806 wherein the polymer is formed from reactants comprising polylysine.

12993. The method of item 12806 wherein the polymer is formed from reactants comprising (a) protein and (b) a compound comprising a polyalkylene oxide portion.

12994. The method of item 12806 wherein the polymer is formed from reactants comprising (a) protein and (b) polylysine.

12995. The method of item 12806 wherein the polymer is formed from reactants comprising (a) protein and (b) a compound having at least four thiol groups.

12996. The method of item 12806 wherein the polymer is formed from reactants comprising (a) protein and (b) a compound having at least four amino groups.

12997. The method of item 12806 wherein the polymer is formed from reactants comprising (a) protein and (b) a compound having at least four carbonyl-oxygen-succinimide groups.

12998. The method of item 12806 wherein the polymer is formed from reactants comprising (a) protein and (b) a compound having a biodegradable region formed from reactants selected from lactic acid, lactide, glycolic acid, glycolide, and epison-caprolactone.

12999. The method of item 12806 wherein the polymer is formed from reactants comprising (a) collagen and (b) a compound comprising a polyalkylene oxide portion.

13000. The method of item 12806 wherein the polymer is formed from reactants comprising (a) collagen and (b) polylysine.

13001. The method of item 12806 wherein the polymer is formed from reactants comprising (a) collagen and (b) a compound having at least four thiol groups.

13002. The method of item 12806 wherein the polymer is formed from reactants comprising (a) collagen and (b) a compound having at least four amino groups.

13003. The method of item 12806 wherein the polymer is formed from reactants comprising (a) collagen and (b) a compound having at least four carbonyl-oxygen-succinimide groups.

13004. The method of item 12806 wherein the polymer is formed from reactants comprising (a) collagen and (b) a compound having a biodegradable region formed from reactants selected from lactic acid, lactide, glycolic acid, glycolide, and epison-caprolactone.

13005. The method of item 12806 wherein the polymer is formed from reactants comprising (a) methylated collagen and (b) a compound comprising a polyalkylene oxide portion.

13006. The method of item 12806 wherein the polymer is formed from reactants comprising (a) methylated collagen and (b) polylysine.

13007. The method of item 12806 wherein the polymer is formed from reactants comprising (a) methylated collagen and (b) a compound having at least four thiol groups.

13008. The method of item 12806 wherein the polymer is formed from reactants comprising (a) methylated collagen and (b) a compound having at least four amino groups.

13009. The method of item 12806 wherein the polymer is formed from reactants comprising (a) methylated collagen and (b) a compound having at least four carbonyl-oxygen-succinimide groups.

13010. The method of item 12806 wherein the polymer is formed from reactants comprising (a) methylated collagen and (b) a compound having a biodegradable region formed from reactants selected from lactic acid, lactide, glycolic acid, glycolide, and epison-caprolactone.

13011. The method of item 12806 wherein the polymer is formed from reactants comprising hyaluronic acid.

13012. The method of item 12806 wherein the polymer is formed from reactants comprising a hyaluronic acid derivative.

13013. The method of item 12806 wherein the polymer is formed from reactants comprising pentaerythritol poly(ethylene glycol)ether tetra-sulfhydryl of number average molecular weight between 3,000 and 30,000.

13014. The method of item 12806 wherein the polymer is formed from reactants comprising pentaerythritol poly(ethylene glycol)ether tetra-amino of number average molecular weight between 3,000 and 30,000.

13015. The method of item 12806 wherein the polymer is formed from reactants comprising (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.

13016. The method of item 12806 wherein the composition comprises a colorant.

13017. The method of item 12806 wherein the composition is sterile.

13018. The method for implanting an electrical device of any one of items 12806-13017 wherein the gastric nerve stimulator is adapted for treating or preventing morbid obesity.

13019. The method for implanting an electrical device of any one of items 12806-13017 wherein the gastric nerve stimulator is adapted for treating or preventing constipation.

13020. The method for implanting an electrical device of any one of items 12806-13017 wherein the gastric nerve stimulator comprises an electrical lead, an electrode and a stimulation generator.

13021. The method for implanting an electrical device of any one of items 12806-13017 wherein the gastric nerve stimulator comprises an electrical signal controller, connector wire and an attachment lead.

13022. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host, wherein the electrical device is a cochlear implant for treating deafness.

13023. The method of item 13022 comprising: (a) infiltrating a tissue of a host where the electrical device is to be, or has been, implanted with an anti-fibrotic agent, and (b) implanting the electrical device into the host.

13024. The method of item 13022 comprising: (a) infiltrating a tissue of a host where the electrical device is to be, or has been, implanted with an anti-infective agent, and (b) implanting the electrical device into the host.

13025. The method of item 13022 comprising: (a) infiltrating a tissue of a host where the electrical device is to be, or has been, implanted with a polymer; and (b) implanting the electrical device into the host.

13026. The method of item 13022 comprising: (a) infiltrating a tissue of a host where the electrical device is to be, or has been, implanted with a composition comprising an anti-fibrotic agent and a polymer, and (b) implanting the electrical device into the host.

13027. The method of item 13022 comprising: (a) infiltrating a tissue of a host where the electrical device is to be, or has been, implanted with a composition comprising an anti-infective agent and a polymer, and (b) implanting the electrical device into the host.

13028. The method of item 13022 comprising: (a) infiltrating a tissue of a host where the electrical device is to be, or has been, implanted with a composition comprising an anti-fibrotic agent, an anti-infective agent and a polymer, and (b) implanting the electrical device into the host.

13029. The method of item 13022 wherein the agent is an adensosine A2A receptor antagonist.

13030. The method of item 13022 wherein the anti-fibrotic agent is an AKT inhibitor.

13031. The method of item 13022 wherein the anti-fibrotic agent is an alpha 2 integrin antagonist, wherein the alpha 2 integrin antagonist is Pharmaprojects No. 5754 (Merck KgaA).

13032. The method of item 13022 wherein the anti-fibrotic agent is an alpha 4 integrin antagonist.

13033. The method of item 13022 wherein the anti-fibrotic agent is an alpha 7 nicotinic receptor agonist.

13034. The method of item 13022 wherein the anti-fibrotic agent is an angiogenesis inhibitor selected from the group consisting of AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), GPA-1734 (Novartis), NNC-47-0011 (Novo Nordisk), herbamycin (Nippon Kayaku), lenalidomide (Celegene), IP-10 (NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zeneca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (Oxigene), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biopharmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), ANGIOCOL (available from Biostratum Inc.), or an analogue or derivative thereof.

13035. The method of item 13022 wherein the anti-fibrotic agent is an apoptosis antagonist.

13036. The method of item 13022 wherein the anti-fibrotic agent is an apoptosis activator.

13037. The method of item 13022 wherein the anti-fibrotic agent is a beta 1 integrin antagonist.

13038. The method of item 13022 wherein the anti-fibrotic agent is a beta tubulin inhibitor.

13039. The method of item 13022 wherein the anti-fibrotic agent is a blocker of enzyme production in Hepatitis C.

13040. The method of item 13022 wherein the anti-fibrotic agent is a Bruton's tyrosine kinase inhibitor.

13041. The method of item 13022 wherein the anti-fibrotic agent is a calcineurin inhibitor.

13042. The method of item 13022 wherein the anti-fibrotic agent is a caspase 3 inhibitor.

13043. The method of item 13022 wherein the anti-fibrotic agent is a CC chemokine receptor antagonist.

13044. The method of item 13022 wherein the anti-fibrotic agent is a cell cycle inhibitor selected from the group consisting of SNS-595 (Sunesis), synthadotin, KRX-0403, and an analogue or derivative thereof.

13045. The method of item 13022 wherein the anti-fibrotic agent is a cathepsin B inhibitor.

13046. The method of item 13022 wherein the anti-fibrotic agent is a cathepsin K inhibitor, wherein the cathepsin K inhibitor is 462795 (GlaxoSmithKline), INPL-022-D6 (Amura Therapeutics), or an analogue or derivative thereof.

13047. The method of item 13022 wherein the anti-fibrotic agent is a cathepsin L inhibitor.

13048. The method of item 13022 wherein the anti-fibrotic agent is a CD40 antagonist.

13049. The method of item 13022 wherein the anti-fibrotic agent is a chemokine receptor agonist.

13050. The method of item 13022 wherein the anti-fibrotic agent is a chymase inhibitor.

13051. The method of item 13022 wherein the anti-fibrotic agent is a collagenase antagonist.

13052. The method of item 13022 wherein the anti-fibrotic agent is a CXCR antagonist.

13053. The method of item 13022 wherein the anti-fibrotic agent is a cyclin dependent kinase inhibitor selected from the group consisting of a CDK-1 inhibitor, a CDK-2 inhibitor, a CDK-4 inhibitor, a CDK-6 inhibitor, a CAK1 inhibitor from GPC Biotech and Bristol-Myers Squibb, RGB-286199 (GPC Biotech), an anticancer agent from Astex Technology, a CAK1 inhibitor from GPC Biotech, a CDK inhibitor from Sanofi-Aventis, a CDK1/CDK2 inhibitor from Amgen, a CDK2 inhibitor from SUGEN-2 (Pfizer), a hearing loss therapy agent (Sound Pharmaceuticals), PD-0332991 (Pfizer), RGB-286199 (GPC Biotech), Ro-0505124 (Hoffmann-La Roche), a Ser/Thr kinase inhibitor from Lilly (Eli Lilly), CVT-2584 (CAS No. 199986-75-9) (CV Therapeutics), and an analogue or derivative thereof.

13054. The method of item 13022 wherein the anti-fibrotic agent is a cyclooxygenase 1 inhibitor.

13055. The method of item 13022 wherein the anti-fibrotic agent is a DHFR inhibitor.

13056. The method of item 13022 wherein the anti-fibrotic agent is a dual integrin inhibitor.

13057. The method of item 13022 wherein the anti-fibrotic agent is an elastase inhibitor.

13058. The method of item 13022 wherein the anti-fibrotic agent is an elongation factor-1 alpha inhibitor.

13059. The method of item 13022 wherein the anti-fibrotic agent is an endothelial growth factor antagonist.

13060. The method of item 13022 wherein the anti-fibrotic agent is an endothelial growth factor receptor kinase inhibitor selected from the group consisting of sorafenib tosylate (Bayer), AAL-993 (Novartis), ABP-309 (Novartis), BAY-57-9352 (Bayer), BIBF-1120 (Boehringer Ingelheim), E-7080 (Eisai), EG-3306 (Ark Therapeutics), EXEL-2880 (Exelixis), GW-654652 (GlaxoSmithKline), a KDR inhibitor from LG Life Sciences, CT-6685 and CT-6729 (UCB), KRN-633 and KRN-951 (Kirin Brewery), OSI-930 (OSI Pharmaceuticals), SP-5.2 (Supratek Pharma), SU-11657 (Pfizer), a Tie-2 antagonist (Hybrigenics), a VEGFR-2 kinase inhibitor (Bristol-Myers Squibb), XL-647 (Exelixis), a KDR inhibitor from Abbott Laboratories, sorafenib tosylate, and an analogue or derivative thereof.

13061. The method of item 13022 wherein the anti-fibrotic agent is an endotoxin antagonist.

13062. The method of item 13022 wherein the anti-fibrotic agent is an epothilone and tubulin binder.

13063. The method of item 13022 wherein the anti-fibrotic agent is an estrogen receptor antagonist.

13064. The method of item 13022 wherein the anti-fibrotic agent is an FGF inhibitor.

13065. The method of item 13022 wherein the anti-fibrotic agent is a farnexyl transferase inhibitor.

13066. The method of item 13022 wherein the anti-fibrotic agent is farnesyltransferase inhibitor selected from the group of A-197574 (Abbott), a farnesyltransferase inhibitor from Servier, FPTIII (Strathclyde Institute for Drug R), LB-42908 (LG Life Sciences), Pharmaprojects No. 5063 (Genzyme), Pharmaprojects No. 5597 (Ipsen), Yissum Project No. B-1055 (Yissum), and an analogue or derivative thereof.

13067. The method of item 13022 wherein the anti-fibrotic agent is an FLT-3 kinase inhibitor.

13068. The method of item 13022 wherein the anti-fibrotic agent is an FGF receptor kinase inhibitor.

13069. The method of item 13022 wherein the anti-fibrotic agent is a fibrinogen antagonist selected from the group consisting of AUV-201 (Auvation), MG-13926 (Sanofi-Aventis), plasminogen activator (CAS No. 105913-11-9) (from Sanofi-Aventis or UCB), plasminogen activator-2 (tPA-2) (Sanofi-Aventis), pro-urokinase (CAS No. 82657-92-9) (Sanofi-Aventis), and an analogue or derivative thereof.

13070. The method of item 13022 wherein the anti-fibrotic agent is a heat shock protein 90 antagonist selected from the group consisting of SRN-005 (Sirenade), geldanamycin, NSC-33050 (17-allylaminogeldanamycin; 17-AAG), 17-dimethylaminoethylamino-17-demethoxy-geldanamycin (17-DMAG), rifabutin (rifamycin XIV, 1′,4-didehydro-1-deoxy-1,4-dihydro-5′-(2-methylpropyl)-1-oxo-), and an analogue or derivative thereof.

13071. The method of item 13022 wherein the anti-fibrotic agent is a histone deacetylase inhibitor.

13072. The method of item 13022 wherein the anti-fibrotic agent is an HMGCoA reductase inhibitor selected from the group consisting of an atherosclerosis therapeutic from Lipid Sciences, ATI-1 6000 (ARYx Therapeutics), KS-01-019 (Kos Pharmaceuticals), Pharmaprojects No. 2197 (Sanofi-Aventi), RP 61969 (Sanofi-Aventis), and an analogue or derivative thereof.

13073. The method of item 13022 wherein the anti-fibrotic agent is an ICAM inhibitor.

13074. The method of item 13022 wherein the anti-fibrotic agent is an IL, ICE and IRAK antagonist, wherein the antagonist is a CJ-14877, CP-424174 (Pfizer), NF-61 (Negma-Lerads), and an analogue or derivative thereof.

13075. The method of item 13022 wherein the anti-fibrotic agent is an IL-2 inhibitor.

13076. The method of item 13022 wherein the anti-fibrotic agent is an immunosuppressant selected from the group consisting of teriflunomide (Sanofi Aventis), chlorsulfaquinoxalone (NSC-339004), chlorsulfaquinoxalone sulfate, CS-712 (Sankyo), ismomultin alfa (CAS No. 457913-93-8) (Akzo Nobel), antiallergics from GenPat77, anti-inflammatories or AT-005 (Androclus Therapeutics), autoimmune disease therapeutics from EpiVax, BN-007 (Bone), budesonide (CAS No. 51333-22-3) (MAP Pharmaceuticals), CO-14 (Genzyme), edratide (CAS No. 433922-67-9) (Teva), EP-314 (Enanta), eprovafen (CAS No. 101335-99-3) (Sanofi-Aventis), HWA-131 (CAS No. 118788-41-3) (Sanofi-Aventis), immunomodulators from MerLion Pharmaceuticals, immunosuppressives from Alchemia, IPL-12 (Inflazyme), MDL-9563 (CAS No. 27086-86-8) (Sanofi-Aventis), Pharmaprojects No. 2330 (Sanofi-Aventis), Pharmaprojects No. 6426 (Abgenix), PXS-25 (Pharmaxis), rosmarinic acid (CAS No. 20283-92-5) (Sanofi-Aventis), RP 42927 or RP 54745 (CAS No. 135330-08-4) (Sanofi-Aventis), SGN-35 (Seattle Genetics), ST-1959 (Sigma-Tau), type I diabetes therapy from SYNX Pharma, UNIL-88 (Debiopharm), VP-025 (Vasogen), VR-694 (Vectura), PRTX-001 (Protalex), and an analogue or derivative thereof.

13077. The method of item 13022 wherein the anti-fibrotic agent is an IMPDH (inosine monophosphate).

13078. The method of item 13022 wherein the anti-fibrotic agent is an integrin antagonist.

13079. The method of item 13022 wherein the anti-fibrotic agent is an interleukin antagonist.

13080. The method of item 13022 wherein the anti-fibrotic agent is an inhibitor of type III receptor tyrosine kinase.

13081. The method of item 13022 wherein the anti-fibrotic agent is an irreversible inhibitor of enzyme methionine aminopeptidase type 2.

13082. The method of item 13022 wherein the anti-fibrotic agent is an isozyme selective delta protein kinase C inhibitor.

13083. The method of item 13022 wherein the anti-fibrotic agent a JAK3 enzyme inhibitor.

13084. The method of item 13022 wherein the anti-fibrotic agent is a JNK inhibitor.

13085. The method of item 13022 wherein the anti-fibrotic agent is a kinase inhibitor.

13086. The method of item 13022 wherein the anti-fibrotic agent is kinesin antagonist.

13087. The method of item 13022 wherein the anti-fibrotic agent is a kinesin antagonist.

13088. The method of item 13022 wherein the anti-fibrotic agent is a leukotriene inhibitor and antagonist selected from the group consisting of ambicromil (CAS No. 58805-38-2) (Sanofi-Aventis), amelubant (CAS No. 346735-24-8) (Boehringer Ingelheim), DW-1141 (Dong Wha), ebselen (Daiichi Pharmaceutical), ibudilast (Kyorin), leucotriene inhibitors from Sanofi-Aventis, lymphotoxin-beta receptor (LT-β) from Biogen Idec, Pharmaprojects No. 1535 and 2728 (CAS No. 119340-33-9) (Sanofi-Aventis), R-112 (Rigel), Rev-5367 (CAS No. 92532-05-3) (Sanofi-Aventis), RG-14893 (CAS No. 141835-49-6) (Sanofi-Aventis), RG-5901-A (CAS No. 101910-24-1), 92532-23-5, RP 66153 (CAS No. 142422-79-5), RP 66364 (CAS No. 186912-92-5), RP 69698 (CAS No. 141748-00-7) (Sanofi-Aventis), SC-411930 (Pfizer), SC-41930 (CAS No. 120072-59-5) (Pfizer), SC-50605 (CAS No. 138828-39-4) (Pfizer), SC-51146 (CAS No. 141059-52-1), SC-53228 (CAS No. 153633-01-3) (Pfizer), spaglumic acid (ZY-1 5106) (CAS No. 3106-85-2), 80619-64-3 (Novartis), tipredane (CAS No. 85197-77-9) (Bristol-Myers Squibb), U-75302 (CAS No. 119477-85-9) (Pfizer), and analogue or derivative thereof.

13089. The method of item 13022 wherein the anti-fibrotic agent is an MAP kinase inhibitor.

13090. The method of item 13022 wherein the anti-fibrotic agent is a matrix metalloproteinase inhibitor.

13091. The method of item 13022 wherein the anti-fibrotic agent is an MCP-CCR2 inhibitor.

13092. The method of item 13022 wherein the anti-fibrotic agent is an mTOR inhibitor.

13093. The method of item 13022 wherein the anti-fibrotic agent is an mTOR kinase inhibitor.

13094. The method of item 13022 wherein the anti-fibrotic agent is a microtubule inhibitor selected from the group consisting of antibody-maytansinoid conjugates from Biogen Idec, colchicines (MantiCore Pharmaceuticals), anticancer immunoconjugates from Johnson & Johnson, DIME from Octamer, gni-1f (GNI), huC242-DM4, huMy9-6-DM1 (ImmunoGen), IDN-5404 (Indena), IMO-098, IMOderm (Imotep), mebendazole (Introgen Therapeutics), microtubule poisons from Cambridge Enterprise, paclitaxel such as LOTAX from Aphios (CAS No. 33069-624), Genexol-PM from Samyang, Pharmaprojects No. 6383 (Tapestry Pharmaceuticals), RPR-112378 (Sanofi-Aventis), SGN-75 (Seattle Genetics), SPL-7435 (Starpharma), SSR-250411 (Sanofi-Aventis), trastuzumab-DM1 (Genentech), vinorelbine, and an analogue or derivative thereof.

13095. The method of item 13022 wherein the anti-fibrotic agent is an MIF inhibitor.

13096. The method of item 13022 wherein the anti-fibrotic agent is an MMP inhibitor.

13097. The method of item 13022 wherein the anti-fibrotic agent is a neurokinin (NK) antagonist selected from the group consisting of anthrotainin (CAS No. 148084-40-6) (Sanofi-Aventis), an IBS therapeutic from ArQule, MDL-105212A (CAS No. 167261-60-1) (Sanofi-Aventis), Pharmaprojects No. 2744, 3258 (CAS No. 139167-47-8) 4006, 4201, or 5986 (Sanofi-Aventis), RP 67580 (CAS No. 135911-02-3), SR-144190 (CAS No. 201152-86-5), SSR-240600, SSR-241586 (Sanofi-Aventis), TKA-457 (Novartis), vestipitant mesylate (CAS No. 334476-64-1) (GlaxoSmithKline), Win-64821 (Sanofi-Aventis), PRX-96026 (Predix Pharmaceuticals), and an analogue or derivative thereof.

13098. The method of item 13022 wherein the anti-fibrotic agent is an NF kappa B inhibitor selected from the group consisting of AVE-0545 or AVE-0547 (Sanofi-Aventis), bortezomib (CAS No. 179324-69-7) (Millennium Pharmaceuticals), (CAS No. 173026-17-0) (OXIS), dexanabinol (CAS No. 112924-45-5) (Pharmos), dexlipotam (Viatris), Pharmaprojects No. 6283 (INDRA) (OXiGENE), IPL-576092 (CAS No. 137571-30-3) (Inflazyme), NFKB decoy (Corgentech), NFKB decoy oligo (AnGes MG), NFKB's from Ariad, osteoporosis treatments or S5 (F005) from Fulcrum Pharmaceuticals, P61 (Phytopharm), R-flurbiprofen (CAS No. 5104-49-4) (Encore Pharmaceuticals), and an analogue or derivative thereof.

13099. The method of item 13022 wherein the anti-fibrotic agent is a nitric oxide agonist.

13100. The method of item 13022 wherein the anti-fibrotic agent is an ornithine decarboxylase inhibitor.

13101. The method of item 13022 wherein the anti-fibrotic agent is a p38 MAP kinase inhibitor selected from the group consisting of AZD-6703 (AstraZeneca), JX-401 (Jexys Pharmaceuticals), BMS-2 (Bristol-Myers Squibb), a p38 MAP kinase inhibitor from Novartis, a p38-alpha MAP kinase inhibitor from Amphora, Pharmaprojects No. 5704 (Pharmacopeia), RPR-200765A (Sanofi-Aventis), SD-282 (Johnson & Johnson), TAK-715 (Takeda), and an analogue or derivative thereof.

13102. The method of item 13022 wherein the anti-fibrotic agent is a palmitoyl-protein thioesterase inhibitor.

13103. The method of item 13022 wherein the anti-fibrotic agent is a PDGF receptor kinase inhibitor selected from the group consisting of AAL-993, AMN-107, or ABP-309 (Novartis), AMG-706 (Amgen), BAY-57-9352 (Bayer), CDP-860 (UCB), E-7080 (Eisai), imatinib (CAS No. 152459-95-5) (Novartis), OSI-930 (OSI Pharmaceuticals), RPR-127963E (Sanofi-Aventis), RWJ-540973 (Johnson & Johnson), sorafenib tosylate (Bayer), SU-11657 (Pfizer), tandutinib (CAS No. 387867-13-2) (Millennium Pharmaceuticals), vatalanib (Novartis), ZK-CDK (Schering AG), and an analogue or derivative thereof.

13104. The method of item 13022 wherein the anti-fibrotic agent is (−)-halofenate (Metabolex), AMG-131 (Amgen), antidiabetics from Japan Tobacco, AZD-4619, AZD-8450, AZD-8677 (AstraZeneca), DRF-10945, balaglitazone (Dr Reddy's), CS-00088, CS-00098 (Chipscreen Biosciences), E-3030 (Eisai), etalocib (CAS No. 161172-51-6) (Eli Lilly), GSK-641597 (Ligand), GSK-677954 (GlaxoSmithKline), GW-409544 (Ligand), GW-590735 (GlaxoSmithKline), K-111 (Hoffmann-La Roche), LY-518674 (Eli Lilly), LY-674 (Ligand), LY-929 (Ligand), MC-3001, MC-3002 (MaxoCore Pharmaceuticals), metformin HCl+pioglitazone (CAS No. 1115-70-4 and 112529-15-4), ACTOPLUS MET from Andrx), muraglitazar (CAS No. 331741-94-7) (Bristol-Myers Squibb), naveglitazar (Ligand), oleoylethanolamide (Kadmus Pharmaceuticals), ONO-5129, pioglitazone hydrochloride (CAS No. 111025-46-8 and 112529-15-4) (Takeda), PLX-204 (Plexxikon), PPAR agonists from Genfit, PPAR delta agonists from Eli Lilly, PPAR-alpha agonists from CrystalGenomics, PPAR-gamma modulators and PPAR-β modulators from C are X, rosiglitazone maleate (CAS No. 122320-73-4 or 155141-29-0) (GlaxoSmithKline), rosiglitazone maleate/glimepir (CAS No. 155141-29-0 and 93479-97-1), AVANDARYL, rosiglitazone maleate/metformin extend (CAS No. 155141-29-0 and 657-24-9), AVANDAMET, rosiglitazone maleate+metformin, AVANDAMET (GlaxoSmithKline), tesaglitazar (AstraZeneca), and an analogue or derivative thereof.

13105. The method of item 13022 wherein the anti-fibrotic agent is a phosphatase inhibitor.

13106. The method of item 13022 wherein the anti-fibrotic agent is a phosphodiesterase (PDE) inhibitor selected from the group consisting of avanafil (Tanabe Seiyaku), dasantafil (CAS No. 569351-91-3) (Schering-Plough), A-906119 (CAS No. 134072-58-5), DL-850 (Sanofi-Aventis), GRC-3015, GRC-3566, GRC-3886 (Glenmark), HWA-153 (CAS No. 56395-66-5) (Sanofi-Aventis), hydroxypumafentrine (Altana), IBFB-130011, IBFB-14-016, IBFB-140301, IBFB-150007, IBFB-211913 (IBFB Pharma), L-826141 (Merck & Co), medorinone (CAS No. 88296-61-1) (Sanofi-Aventis), MEM-1917 (Memory Pharmaceuticals), ND-1251 (Neuro3d), PDE inhibitors from ICOS, PDE IV inhibitors from Memory Pharmaceuticals and CrystalGenomics, Pharmaprojects No. 2742 and 6141 (Sanofi-Aventis), QAD-171 (Novartis), RHC-2963 (CAS No. 76993-12-9 and 76993-14-1), RPR-117658, RPR-122818 derivatives, SR-24870, and RPR-132294 (Sanofi-Aventis), SK-350 (In2Gen), stroke therapy agents from deCODE Genetics, TAS-203 (Taiho), tofimilast (CAS No. 185954-27-2) (Pfizer), UK-371800 (Pfizer), WIN-65579 (CAS No. 158020-82-7) (Sanofi-Aventis), IBFB-130020 (IBFB Pharma), OPC-6535 (CAS No. 145739-56-6) (Otsuka), a phosphodiesterase III inhibitor, enoximone, a phosphodiesterase IV inhibitor, fosfosal, Atopik (Barrier Therapeutics), triflusal, a phosphodiesterase V inhibitor, and an analogue or derivative thereof.

13107. The method of item 13022 wherein the anti-fibrotic agent is a PKC inhibitor.

13108. The method of item 13022 wherein the anti-fibrotic agent is a platelet activating factor antagonist.

13109. The method of item 13022 wherein the anti-fibrotic agent is a platelet-derived growth factor receptor kinase inhibitor.

13110. The method of item 13022 wherein the anti-fibrotic agent is a prolyl hydroxylase inhibitor.

13111. The method of item 13022 wherein the anti-fibrotic agent is a polymorphonuclear neutrophil inhibitor.

13112. The method of item 13022 wherein the anti-fibrotic agent is a protein kinase B inhibitor.

13113. The method of item 13022 wherein the anti-fibrotic agent is a protein kinase C stimulant.

13114. The method of item 13022 wherein the anti-fibrotic agent is a purine nucleoside analogue.

13115. The method of item 13022 wherein the anti-fibrotic agent is a purinoreceptor P2X antagonist.

13116. The method of item 13022 wherein the anti-fibrotic agent is a Raf kinase inhibitor.

13117. The method of item 13022 wherein the anti-fibrotic agent is a reversible inhibitor of ErbB1 and ErbB2.

13118. The method of item 13022 wherein the anti-fibrotic agent is a ribonucleoside triphosphate reductase inhibitor.

13119. The method of item 13022 wherein the anti-fibrotic agent is an SDF-1 antagonist.

13120. The method of item 13022 wherein the anti-fibrotic agent is a sheddase inhibitor.

13121. The method of item 13022 wherein the anti-fibrotic agent is an SRC inhibitor.

13122. The method of item 13022 wherein the anti-fibrotic agent is a stromelysin inhibitor.

13123. The method of item 13022 wherein the anti-fibrotic agent is an Syk kinase inhibitor.

13124. The method of item 13022 wherein the anti-fibrotic agent is a telomerase inhibitor.

13125. The method of item 13022 wherein the anti-fibrotic agent is a TGF beta inhibitor selected from the group consisting of pirfenidone (CAS No. 53179-13-8) (MARNAC), tranilast (CAS No. 53902-12-8) (Kissei), IN-1130 (In2Gen), mannose-6-phosphate (BTG), TGF-β antagonists from Inflazyme (Pharmaprojects No. 6075), TGF-β antagonists from Sydney, non-industrial source), TGF-βI receptor kinase inhibitors from Eli Lilly, TGF-β receptor inhibitors from Johnson & Johnson, and an analogue or derivative thereof.

13126. The method of item 13022 wherein the anti-fibrotic agent is a TNFα antagonist or TACE inhibitor selected from the group consisting of adalimumab (CAS No. 331731-18-1) (Cambridge Antibody Technology), AGIX-4207 (AtheroGenics), AGT-1 (Advanced Biotherapy), an anti-inflammatory from Borean Pharma, Cellzome, or Paradigm Therapeutics, anti-inflammatory vaccine (TNF-alpha kinoid) from Neovacs, humanized anti-TNF antibody or an anti-TNF MAb (CB0006) Celltech (UCB), apratastat (CAS No. 287405-51-0) (Wyeth), BMS-561392 (Bristol-Myers Squibb), BN-006 (Bone), certolizumab pegol (CAS No. 428863-50-7 or CH-138 (UCB), cilomilast (CAS No. 153259-65-5) (GlaxoSmithKline), CR-1 (Nuada Pharmaceuticals), CRx-119 (CombinatoRx), D-5410 (UCB), dacopafant (CAS No. 125372-33-0) (Sanofi-Aventis), dersalazine (CAS No. 188913-57-7/188913-58-8) (Uriach), etanercept (CAS No. 185243-69-0) (Amgen), ethyl pyruvate (Critical (Critical Therapeutics), golimumab (CAS No. 476181-74-5) (Johnson & Johnson), hormono-immunotherapy from Ipsen, CDP571 (e.g., Humicade from UCB), IC-485 (ICOS), infliximab (CAS No. 170277-31-3) (Johnson & Johnson), IP-751 (Manhattan Pharmaceuticals), ISIS-104838 (CAS No. 250755-32-9) (ISIS Pharmaceuticals), lenalidomide (CAS No. 191732-72-6) (Celgene), lentinan (CAS No. 37339-90-5) (Ajinomoto), MDL-201112 (CAS No. 142130-73-2) (Sanofi-Aventis), medroxyprogesterone (CAS No. 520-85-4) (InKine Pharmaceutical), N-acetylcysteine (CAS No. 616-91-1) (Zambon), NBE-P2 (DIREVO Biotech), nerelimomab (CAS No. 162774-06-3) (Chiron), OM-294DP (OM PHARMA), onercept (CAS No. 199685-57-9) (Yeda), PASSTNF-alpha (Verigen), pentoxifylline or oxypentifylline (Sanofi-Aventis), Pharmaprojects No. 4091, 4241, 4295, or 4488 (Sanofi-Aventis), Pharmaprojects No. 5480 (Amgen), Pharmaprojects No. 6749 (Cengent), pirfenidone (CAS No. 53179-13-8) (MARNAC), RPR-132294 (Sanofi-Aventis), S5 (F002) (Fulcrum Pharmaceuticals), simvastatin (CAS No. 79902-63-9) (Merck & Co), STA-6292 (Synta Pharmaceuticals), tacrolimus (CAS No. 104987-11-3) (Fujisawa LifeCycle Pharma), talactoferrin alfa (CAS No. 308240-58-6) (Agennix), thalidomide (CAS No. 50-35-1) (Celgene), TNF antagonists form ProStrakan, and Synergen, TNF inhibitors (Amgen), TNF-alpha antagonists from Dynavax Technologies and Jerina AG (Germany), TNF-alpha inhibitors from IBFB Pharma and Xencor (Xencor), torbafylline (CAS No. 105102-21-4) (Sanofi-Aventis), UR-1505 (Uriach), VT-346 (Viron Therapeutics), YSIL6 (Y's Therapeutics), YSTH2 (Y's Therapeutics), NPI-1302a-3 (Nereus Pharmaceuticals, a TNF antagonist from Jerina AG (Germany), dersalazine, and an analogue or derivative thereof.

13127. The method of item 13022 wherein the anti-fibrotic agent is a Toll receptor inhibitor.

13128. The method of item 13022 wherein the anti-fibrotic agent is a tubulin antagonist.

13129. The method of item 13022 wherein the anti-fibrotic agent is a tyrosine kinase inhibitor selected from the group consisting of SU-011248, SUTENT from Pfizer Inc. (New York, N.Y.), BMS-354825, PN-355 (Paracelsian Pharmaceuticals), AGN-199659 (Allergan), AAL-993 or ABP-309 (Novartis), adaphostin (NIH), AEE-788 (Novartis), AG-013736 (OSI Pharmaceuticals), AG-13736 (Pfizer), ALT-110 (Alteris Therapeutics), AMG-706 (Amgen), anticancer MAbs from Xencor, anti-EGFrvIII MAbs from Abgenix, anti-HER2 MAb from Abiogen, AZD-2171 or AZD-9935 (AstraZeneca), BAY-57-9352 (Bayer), BIBF-1120 (Boehringer Ingelheim), CEP-5214 (Cephalon), CEP-7055 (Cephalon), cetuximab (ImClone Systems), CHIR-200131 and CHIR-258 (Chiron), CP-547632 (OSI Pharmaceuticals), CP-724714 (Pfizer), CT-301 (Creabilis Therapeutics), D-69491 (Baxter International), E-7080 (Eisai), EG-3306 (Ark Therapeutics), EGFR/ErbB2 inhibitors from Array BioPharma, erlotinib (CAS No. 183319-69-9) (OSI Pharmaceuticals), EXEL-2880 (Exelixis), FK-778 (Sanofi-Aventis), gefitinib (CAS No. 184475-35-2) (AstraZeneca), GW-2286 or GW-654652 (GlaxoSmithKline), her2/neu antigen from AlphaVax, HER-2/neu inhibitor from Generex, Herzyme (Medipad) (Sirna Therapeutics), HKI-272 (Wyeth), HuMax-EGFr (Genmab), idronoxil (CAS No. 81267-65-4) (Novogen), IGF-1 inhibitors from Ontogen, IMC-11F8 (ImClone Systems), kahalalide F (CAS No. 149204-42-2) (PharmaMar), KDR inhibitor from LG Life Sciences, KDR inhibitors from Abbott Laboratories, KDR kinase inhibitors (UCB), Kdr kinase inhibitors from Merck & Co, KRN-633 and KRN-951 (Kirin Brewery), KSB-102 (Xenova), lapatinib ditosylate (CAS No. 388082-78-8) (GlaxoSmithKline), matuzumab (Merck KGaA), MDX-214 (Medarex), ME-103 (Pharmexa), MED-A300 (Gerolymatos), MNAC-13 (Lay Line Genomics), nimotuzumab (Center of Molecular Immunology), NSC-330507 or NSC-707545 (NIH), NV-50 (Novogen), OSI-930 (OSI Pharmaceuticals), panitumumab (Abgenix), pelitinib (CAS No. 287933-82-7) (Wyeth), pertuzumab (CAS No. 380610-27-5) (Genentech), Pharmaprojects No. 3985 (Sanofi-Aventis), prostate cancer therapeutics from Sequenom (SQPC35, SQPC36, SQPC90), removab and remoxab (Trion Pharma), RG-13022 (CAS No. 136831-48-6), RG-13291 (CAS No. 138989-50-1), or RG-14620 (CAS No. 136831-49-7) (Sanofi-Aventis), RM-6427 (Romark), RNAi breast cancer therapy from Benitec, RP 53801 (CAS No. 125882-88-4) (Sanofi-Aventis), sorafenib tosylate (Bayer), SU-11657 (Pfizer), Tie-2 antagonists from Semaia (Hybrigenics), Tie-2 inhibitors from Ontogen, trastuzumab (CAS No. 180288-69-1) (Genentech), tyrosine kinase inhibitors from Sanofi-Aventis, U3-1287, U3-1565, U3-1784, U3-1800 (U3 Pharma), vatalanib (Novartis), VEGFR-2 kinase inhibitor from Bristol-Myers Squibb, XL-647 (Exelixis), ZD-6474 (AstraZeneca), ZK-CDK (Schering AG), an EGFR tyrosine kinase inhibitor, EKB-569 (Wyeth), and an analogue or derivative thereof.

13130. The method of item 13022 wherein the anti-fibrotic agent is a VEGF inhibitor.

13131. The method of item 13022 wherein the anti-fibrotic agent is a vitamin D receptor agonist.

13132. The method of item 13022 wherein the anti-fibrotic agent is ZD-6474 (an angiogenesis inhibitor).

13133. The method of item 13022 wherein the anti-fibrotic agent is AP-23573 (an mTOR inhibitor).

13134. The method of item 13022 wherein the anti-fibrotic agent is synthadotin (a tubulin antagonist).

13135. The method of item 13022 wherein the anti-fibrotic agent is S-0885 (a collagenase inhibitor).

13136. The method of item 13022 wherein the anti-fibrotic agent is aplidine (an elongation factor-1 alpha inhibitor).

13137. The method of item 13022 wherein the anti-fibrotic agent is ixabepilone (an epithilone).

13138. The method of item 13022 wherein the anti-fibrotic agent is IDN-5390 (an angiogenesis inhibitor).

13139. The method of item 13022 wherein the anti-fibrotic agent is SB-2723005 (an angiogenesis inhibitor).

13140. The method of item 13022 wherein the anti-fibrotic agent is ABT-518 (an angiogenesis inhibitor).

13141. The method of item 13022 wherein the anti-fibrotic agent is combretastatin (an angiogenesis inhibitor).

13142. The method of item 13022 wherein the anti-fibrotic agent is anecortave acetate (an angiogenesis inhibitor).

13143. The method of item 13022 wherein the anti-fibrotic agent is SB-715992 (a kinesin antagonist).

13144. The method of item 13022 wherein the anti-fibrotic agent is temsirolimus (an mTOR inhibitor).

13145. The method of item 13022 wherein the anti-fibrotic agent is adalimumab (a TNFα antagonist).

13146. The method of item 13022 wherein the anti-infective agent is an anthracycline.

13147. The method of item 13022 wherein the anti-infective agent is doxorubicin.

13148. The method of item 13022 wherein the anti-infective agent is mitoxantrone.

13149. The method of item 13022 wherein the anti-infective agent is a fluoropyrimidine.

13150. The method of item 13022 wherein the anti-infective agent is 5-fluorouracil (5-FU).

13151. The method of item 13022 wherein the anti-infective agent is a folic acid antagonist.

13152. The method of item 13022 wherein the anti-infective agent is methotrexate.

13153. The method of item 13022 wherein the anti-infective agent is a podophylotoxin.

13154. The method of item 13022 wherein the anti-infective agent is etoposide.

13155. The method of item 13022 wherein the anti-infective agent is camptothecin.

13156. The method of item 13022 wherein the anti-infective agent is a hydroxyurea.

13157. The method of item 13022 wherein the anti-infective agent is a platinum complex.

13158. The method of item 13022 wherein the anti-infective agent is cisplatin.

13159. The method of item 13022 wherein the composition comprises an anti-thrombotic agent.

13160. The method of item 13022 wherein the polymer is formed from reactants comprising a naturally occurring polymer.

13161. The method of item 13022 wherein the polymer is formed from reactants comprising protein.

13162. The method of item 13022 wherein the polymer is formed from reactants comprising carbohydrate.

13163. The method of item 13022 wherein the polymer is formed from reactants comprising biodegradable polymer.

13164. The method of item 13022 wherein the polymer is formed from reactants comprising nonbiodegradable polymer.

13165. The method of item 13022 wherein the polymer is formed from reactants comprising collagen.

13166. The method of item 13022 wherein the polymer is formed from reactants comprising methylated collagen.

13167. The method of item 13022 wherein the polymer is formed from reactants comprising fibrinogen.

13168. The method of item 13022 wherein the polymer is formed from reactants comprising thrombin.

13169. The method of item 13022 wherein the polymer is formed from reactants comprising blood plasma.

13170. The method of item 13022 wherein the polymer is formed from reactants comprising calcium salt.

13171. The method of item 13022 wherein the polymer is formed from reactants comprising an antifibronolytic agent.

13172. The method of item 13022 wherein the polymer is formed from reactants comprising fibrinogen analog.

13173. The method of item 13022 wherein the polymer is formed from reactants comprising albumin.

13174. The method of item 13022 wherein the polymer is formed from reactants comprising plasminogen.

13175. The method of item 13022 wherein the polymer is formed from reactants comprising von Willebrands factor.

13176. The method of item 13022 wherein the polymer is formed from reactants comprising Factor VIII.

13177. The method of item 13022 wherein the polymer is formed from reactants comprising hypoallergenic collagen.

13178. The method of item 13022 wherein the polymer is formed from reactants comprising atelopeptidic collagen.

13179. The method of item 13022 wherein the polymer is formed from reactants comprising telopeptide collagen.

13180. The method of item 13022 wherein the polymer is formed from reactants comprising crosslinked collagen.

13181. The method of item 13022 wherein the polymer is formed from reactants comprising aprotinin.

13182. The method of item 13022 wherein the polymer is formed from reactants comprising epsilon-amino-n-caproic acid.

13183. The method of item 13022 wherein the polymer is formed from reactants comprising gelatin.

13184. The method of item 13022 wherein the polymer is formed from reactants comprising protein conjugates.

13185. The method of item 13022 wherein the polymer is formed from reactants comprising gelatin conjugates.

13186. The method of item 13022 wherein the polymer is formed from reactants comprising a synthetic polymer.

13187. The method of item 13022 wherein the polymer is formed from reactants comprising a synthetic isocyanate-containing compound.

13188. The method of item 13022 wherein the polymer is formed from reactants comprising a synthetic thiol-containing compound.

13189. The method of item 13022 wherein the polymer is formed from reactants comprising a synthetic compound containing at least two thiol groups.

13190. The method of item 13022 wherein the polymer is formed from reactants comprising a synthetic compound containing at least three thiol groups.

13191. The method of item 13022 wherein the polymer is formed from reactants comprising a synthetic compound containing at least four thiol groups.

13192. The method of item 13022 wherein the polymer is formed from reactants comprising a synthetic amino-containing compound.

13193. The method of item 13022 wherein the polymer is formed from reactants comprising a synthetic compound containing at least two amino groups.

13194. The method of item 13022 wherein the polymer is formed from reactants comprising a synthetic compound containing at least three amino groups.

13195. The method of item 13022 wherein the polymer is formed from reactants comprising a synthetic compound containing at least four amino groups.

13196. The method of item 13022 wherein the polymer is formed from reactants comprising a synthetic compound comprising a carbonyl-oxygen-succinimidyl group.

13197. The method of item 13022 wherein the polymer is formed from reactants comprising a synthetic compound comprising at least two carbonyl-oxygen-succinimidyl groups.

13198. The method of item 13022 wherein the polymer is formed from reactants comprising a synthetic compound comprising at least three carbonyl-oxygen-succinimidyl groups.

13199. The method of item 13022 wherein the polymer is formed from reactants comprising a synthetic compound comprising at least four carbonyl-oxygen-succinimidyl groups.

13200. The method of item 13022 wherein the polymer is formed from reactants comprising a synthetic polyalkylene oxide-containing compound.

13201. The method of item 13022 wherein the polymer is formed from reactants comprising a synthetic compound comprising both polyalkylene oxide and biodegradable polyester blocks.

13202. The method of item 13022 wherein the polymer is formed from reactants comprising a synthetic polyalkylene oxide-containing compound having reactive amino groups.

13203. The method of item 13022 wherein the polymer is formed from reactants comprising a synthetic polyalkylene oxide-containing compound having reactive thiol groups.

13204. The method of item 13022 wherein the polymer is formed from reactants comprising a synthetic polyalkylene oxide-containing compound having reactive carbonyl-oxygen-succinimidyl groups.

13205. The method of item 13022 wherein the polymer is formed from reactants comprising a synthetic compound comprising a biodegradable polyester block.

13206. The method of item 13022 wherein the polymer is formed from reactants comprising a synthetic polymer formed in whole or part from lactic acid or lactide.

13207. The method of item 13022 wherein the polymer is formed from reactants comprising a synthetic polymer formed in whole or part from glycolic acid or glycolide.

13208. The method of item 13022 wherein the polymer is formed from reactants comprising polylysine.

13209. The method of item 13022 wherein the polymer is formed from reactants comprising (a) protein and (b) a compound comprising a polyalkylene oxide portion.

13210. The method of item 13022 wherein the polymer is formed from reactants comprising (a) protein and (b) polylysine.

13211. The method of item 13022 wherein the polymer is formed from reactants comprising (a) protein and (b) a compound having at least four thiol groups.

13212. The method of item 13022 wherein the polymer is formed from reactants comprising (a) protein and (b) a compound having at least four amino groups.

13213. The method of item 13022 wherein the polymer is formed from reactants comprising (a) protein and (b) a compound having at least four carbonyl-oxygen-succinimide groups.

13214. The method of item 13022 wherein the polymer is formed from reactants comprising (a) protein and (b) a compound having a biodegradable region formed from reactants selected from lactic acid, lactide, glycolic acid, glycolide, and epison-caprolactone.

13215. The method of item 13022 wherein the polymer is formed from reactants comprising (a) collagen and (b) a compound comprising a polyalkylene oxide portion.

13216. The method of item 13022 wherein the polymer is formed from reactants comprising (a) collagen and (b) polylysine.

13217. The method of item 13022 wherein the polymer is formed from reactants comprising (a) collagen and (b) a compound having at least four thiol groups.

13218. The method of item 13022 wherein the polymer is formed from reactants comprising (a) collagen and (b) a compound having at least four amino groups.

13219. The method of item 13022 wherein the polymer is formed from reactants comprising (a) collagen and (b) a compound having at least four carbonyl-oxygen-succinimide groups.

13220. The method of item 13022 wherein the polymer is formed from reactants comprising (a) collagen and (b) a compound having a biodegradable region formed from reactants selected from lactic acid, lactide, glycolic acid, glycolide, and epison-caprolactone.

1. A medical device, comprising an electrical device and an anti-scarring agent or a composition comprising an anti-scarring agent, wherein the agent inhibits scarring between the medical device and the host into which the medical device is implanted.

2. The medical device of claim 1 wherein the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

3. The medical device of claim 1 or claim 2 wherein the anti-scarring agent is an antimicrobial compound.

4. The medical device of claim 3 wherein the antimicrobial compound is brefeldin A.

5. The medical device of claim 1 or claim 2 wherein the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

6. The medical device of claim 1 or claim 2 wherein the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

7. The medical device of claim 1 or claim 2 wherein the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

8. The medical device of claim 1 or claim 2 wherein the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

9. The medical device of any one of claims 1-8 further comprising a coating wherein the coating comprises (a) the anti-scarring agent or (b) the anti-scarring agent and a polymer.

10. The medical device of any one of claims 1-8 further comprising a coating wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

11. The medical device of any one of claims 1-8 further comprising a polymer or further comprising a polymeric carrier.

12. The medical device of claim 11 wherein the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

13. The medical device of any one of claims 1-8 further comprising a polymeric carrier, wherein the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

14. The medical device of any one of claims 1-8 further comprising a second pharmaceutically active agent.

15. The medical device of any one of claims 1-8 further comprising at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

16. The medical device of any one of claims 1-8 wherein the device is adapted for delivering the anti-scarring agent locally to tissue proximate to the device.

17. The medical device of any one of claims 1-8 wherein the anti-scarring agent is released into tissue in the vicinity of the device after deployment of the device.

18. The medical device of any one of claims 1-8 wherein the anti-scarring agent is released in effective concentrations from the device over a period ranging from the time of deployment of the device to about 1 year.

19. The medical device of any one of claims 1-8 wherein the anti-scarring agent is released in effective concentrations from the device at a constant rate, an increasing rate, or a decreasing rate.

20. The medical device of any one of claims 1-8 wherein the device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

21. The medical device of any one of claims 1-8 wherein the device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

22. The medical device of any one of claims 1-8 wherein the device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

23. A method for inhibiting scarring comprising placing an electrical device and an anti-scarring agent or a composition comprising an ant-scarring agent into an animal host, wherein the agent inhibits scarring.

24. The method of claim 23 wherein the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

25. The method of claim 23 or claim 24 wherein the anti-scarring agent is an antimicrobial compound.

26. The method of claim 25 wherein the antimicrobial compound is brefeldin A.

27. The method of claim 23 or claim 24 wherein the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

28. The method of claim 23 or claim 24 wherein the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

29. The method of claim 23 or claim 24 wherein the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

30. The method of claim 23 or claim 24 wherein the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

31. The method of any one of claims 23-30 wherein the electrical device further comprises a coating, and wherein the coating comprises (a) the anti-scarring agent or (b) the anti-scarring agent and a polymer.

32. The method of any one of claims 23-30 wherein the electrical device further comprises a coating, and wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

33. The method of any one of claims 23-30 wherein the electrical device further comprises a polymer or further comprises a polymeric carrier.

34. The method of claim 33 wherein the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

35. The method of any one of claims 23-30 wherein the electrical device further comprises a polymeric carrier, and wherein the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

36. The method of any one of claims 23-30 wherein the electrical device further comprises a second pharmaceutically active agent.

37. The method of any one of claims 23-30 wherein the electrical device further comprises at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

38. The method of any one of claims 23-30 wherein the electrical device is adapted for delivering the anti-scarring agent locally to tissue proximate to the device.

39. The method of any one of claims 23-30 wherein the anti-scarring agent is released into tissue in the vicinity of the electrical device after deployment of the device.

40. The method of any one of claims 23-30 wherein the anti-scarring agent is released in effective concentrations from the electrical device over a period ranging from the time of deployment of the device to about 1 year.

41. The method of any one of claims 23-30 wherein the anti-scarring agent is released in effective concentrations from the electrical device at a constant rate, an increasing rate, or a decreasing rate.

42. The method of any one of claims 23-30 wherein the electrical device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

43. The method of any one of claims 23-30 wherein the electrical device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

44. The method of any one of claims 23-30 wherein the electrical device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

45. A method for making a medical device comprising: combining an electrical device and an anti-scarring agent or a composition comprising an anti-scarring agent, wherein the agent inhibits scarring between the device and a host into which the device is implanted.

46. The method of claim 45 wherein the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

47. The method of claim 45 or claim 46 wherein the anti-scarring agent is an antimicrobial compound.

48. The method of claim 47 wherein the antimicrobial compound is brefeldin A.

49. The method of claim 45 or claim 46 wherein the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

50. The method of claim 45 or claim 46 wherein the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase I inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

51. The method of claim 45 or claim 46 wherein the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

52. The method of claim 45 or claim 46 wherein the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

53. The method of any one of claims 45-52 wherein the electrical device further comprises a coating, and wherein the coating comprises (a) the anti-scarring agent or (b) the anti-scarring agent and a polymer.

54. The method of any one of claims 45-52 wherein the electrical device further comprises a coating, and wherein the anti-scarring agent is present in the coating in an amount ranging between (a) about 0.0001% to about 1% by weight; (b) about 1% to about 10% by weight; (c) about 10% to about 25% by weight; or (d) about 25% to about 70% by weight.

55. The method of any one of claims 45-52 wherein the electrical device further comprises a polymer or further comprises a polymeric carrier.

56. The method of claim 55 wherein the polymeric carrier comprises a copolymer, a block copolymer, a random copolymer, a biodegradable polymer, a non-biodegradable polymer, a hydrophilic polymer, a hydrophobic polymer, a polymer having hydrophilic domains, or a polymer having hydrophobic domains.

57. The method of any one of claims 45-52 wherein the electrical device further comprises a polymeric carrier, and wherein the polymeric carrier comprises a non-conductive polymer, an elastomer, a hydrogel, a silicone polymer, a hydrocarbon polymer, a styrene-derived polymer, a butadiene polymer, a macromer, a poly-ethylene glycol) polymer, and an amorphous polymer.

58. The method of any one of claims 45-52 wherein the electrical device further comprises a second pharmaceutically active agent.

59. The method of any one of claims 45-52 wherein the electrical device further comprises at least one of an anti-inflammatory agent, an agent that inhibits infection, anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, cisplatin, an anti-thrombotic agent, a visualization agent, and an echogenic material.

60. The method of any one of claims 45-52 wherein the electrical device is adapted for delivering the anti-scarring agent locally to tissue proximate to the device.

61. The method of any one of claims 45-52 wherein the anti-scarring agent is released into tissue in the vicinity of the electrical device after deployment of the device.

62. The method of any one of claims 45-52 wherein the anti-scarring agent is released in effective concentrations from the electrical device over a period ranging from the time of deployment of the device to about 1 year.

63. The method of any one of claims 45-52 wherein the anti-scarring agent is released in effective concentrations from the electrical device at a constant rate, an increasing rate, or a decreasing rate.

64. The method of any one of claims 45-52 wherein the electrical device comprises (a) about 0.01 μg to about 10 μg of the anti-scarring agent; (b) about 10 μg to about 10 mg of the anti-scarring agent; (c) about 10 mg to about 250 mg of the anti-scarring agent; (d) about 250 mg to about 1000 mg of the anti-scarring agent; or (e) about 1000 mg to about 2500 mg of the anti-scarring agent.

65. The method of any one of claims 45-52 wherein the electrical device comprises (a) about 0.01 μg to about 1 μg of the anti-scarring agent per mm² of device surface to which the anti-scarring agent is applied; (b) about 1 μg to about 10 μg of the anti-scarring agent per mm² of device surface to which the anti-scarring agent is applied; (c) about 10 μg to about 250 μg of the anti-scarring agent per mm² of device surface to which the anti-scarring agent is applied; (d) about 250 μg to about 1000 μg of the anti-scarring agent per mm² of device surface to which the anti-scarring agent is applied; or (e) about 1000 μg to about 2500 μg of the anti-scarring agent per mm² of device surface to which the anti-scarring agent is applied.

66. The method of any one of claims 45-52 wherein the electrical device comprises (a) about 0.01 μg to about 100 μg of the anti-scarring agent per mm² of device surface to which the anti-scarring agent is applied; (b) about 0.01 μg to about 200 μg of the anti-scarring agent per mm² of device surface to which the anti-scarring agent is applied; or (c) about 0.01 μg to about 500 μg of the anti-scarring agent per mm² of device surface to which the anti-scarring agent is applied.

67. A method for implanting an electrical device comprising: (a) infiltrating a tissue of a host where the electrical device is to be, or has been, implanted with i) an anti-fibrotic agent, ii) an anti-infective agent, iii) a polymer; iv) a composition comprising an anti-fibrotic agent and a polymer, v) a composition comprising an anti-infective agent and a polymer, or vi) a composition comprising an anti-fibrotic agent, an anti-infective agent and a polymer, and (b) implanting the electrical device into the host.

68. The method of claim 67 wherein the electrical device is a neurostimulator for treating chronic pain, a neurostimulator for treating Parkinson's Disease, a vagal nerve stimulator for treating epilepsy, a vagal nerve stimulator for treating a chronic or degenerative neurological disorder, a sacral nerve stimulator for treating a bladder control problem, a gastric nerve stimulator for treating a gastrointestinal disorder, a cochlear implant for treating deafness, a bone growth stimulator, a cardiac pacemaker, an implantable cardioverter defibrillator (ICD) system, a vagus nerve stimulator for treating arrhythemia, an electrical lead, a neurostimulator, or a cardiac rhythm management device.

69. The method of claim 67 or claim 68 wherein the anti-scarring agent is an antimicrobial compound.

70. The method of claim 69 wherein the antimicrobial compound is brefeldin A.

71. The method of claim 67 or claim 68 wherein the anti-scarring agent is selected from a histamine receptor antagonist, an alpha adrenergic receptor antagonist, an anti-psychotic compound, a CaM kinase II inhibitor, a G protein agonist, an antibiotic selected from the group consisting of apigenin, ampicillin sodium salt, puromycin, an anti-microbial agent, a DNA topoisomerase inhibitor, a thromboxane A2 receptor inhibitor, a D2 dopamine receptor antagonist, a Peptidyl-Prolyl Cis/Trans Isomerase Inhibitor, a dopamine antagonist, an anesthetic compound, a clotting factor, a lysyl hydrolase inhibitor, a muscarinic receptor inhibitor, a superoxide anion generator, a steroid, an anti-proliferative agent, a diuretic, an anti-coagulant, a cyclic GMP agonist, an adenylate cyclase agonist, an antioxidant, a nitric oxide synthase inhibitor, an anti-neoplastic agent, a DNA synthesis inhibitor, a DNA alkylating agent, a DNA methylation inhibitor, a NSAID agent, a peptidylglycine alpha-hydroxylating monooxygenase inhibitor, an MEK1/MEK 2 inhibitor, a NO synthase inhibitor, a retinoic acid receptor antagonist, an ACE inhibitor, a glycosylation inhibitor, an intracellular calcium influx inhibitor, an anti-emetic agent, an acetylcholinesterase inhibitor, an ALK-5 receptor antagonist, a RAR/RXT antagonist, an eIF-2a inhibitor, an S-adenosyl-L-homocysteine hydrolase inhibitor, an estrogen agonist, a serotonin receptor inhibitor, an anti-thrombotic agent, a tryptase inhibitor, a pesticide, a bone mineralization promoter, a bisphosphonate compound selected from risedronate and an analogue or derivative thereof, an anti-inflammatory compound, a DNA methylation promoter, an anti-spasmodic agent, a protein synthesis inhibitor, an α-glucosidase inhibitor, a calcium channel blocker, a pyruvate dehydrogenase activator, a prostaglandin inhibitor, a sodium channel inhibitor, a serine protease inhibitor, an intracellular calcium flux inhibitor, a JAK2 inhibitor, an androgen inhibitor, an aromatase inhibitor, an anti-viral agent, a 5-HT inhibitor, an FXR antagonist, an actin polymerization and stabilization promoter, an AXOR12 agonist, an angiotensin II receptor agonist, a platelet aggregation inhibitor, a CB1/CB2 receptor agonist, a norepinephrine reuptake inhibitor, a selective serotonin reuptake inhibitor, a reducing agent, and a immuno-modulator selected from Bay 11-7085, (−)-arctigenin, idazoxan hydrochloride.

72. The method of claim 67 or claim 68 wherein the anti-scarring agent is selected from an angiogenesis inhibitor, an apoptosis antagonist, an apoptosis activator, a beta 1 integrin antagonist, a beta tubulin inhibitor, a blocker of enzyme production in Hepatitis C, a Bruton's tyrosine kinase inhibitor, a calcineurin inhibitor, a caspase 3 inhibitor, a CC chemokine receptor antagonist, a cell cycle inhibitor, a cathepsin B inhibitor, a cathepsin K inhibitor, a cathepsin L inhibitor, a CD40 antagonist, a chemokine receptor antagonist, a chymase inhibitor, a collagenase antagonist, a CXCR antagonist, a cyclin dependent kinase inhibitor, a cyclooxygenase 1 inhibitor, a DHFR inhibitor, a cual integrin inhibitor, an elastase inhibitor, an elongation factor-1 alpha inhibitor, an endothelial growth factor antagonist, an endothelial growth factor receptor kinase inhibitor, an endotoxin antagonist, an epothilone and tubulin binder, an estrogen receptor antagonist, an FGF inhibitor, a farnexyl transferase inhibitor, a farnesyltransferase inhibitor, an FLT-3 kinase inhibitor, an FGF receptor kinase inhibitor, a fibrinogen antagonist, a histone deacetylase inhibitor, an HMGCoA reductase inhibitor, an ICAM inhibitor, an IL, ICE, and IRAK antagonist, an IL-2 inhibitor, an immunosuppressant, an inosine monophosphate inhibitor, an integrin antagonist, an interleukin antagonist, an inhibitor of type III receptor tyrosine kinase, an irreversible inhibitor of enzyme methionine aminopeptidase type 2, an isozyme selective delta protein kinase C inhibitor, a JAK3 enzyme inhibitor, a JNK inhibitor, a kinase inhibitor, a kinesin antagonist, a leukotriene inhibitor and antagonist, a MAP kinase inhibitor, a matrix metalloproteinase inhibitor, an MCP-CCR2 inhibitor, an mTOR inhibitor, an mTOR kinase inhibitor, a microtubule inhibitor, an MIF inhibitor, a neurokinin antagonist, an NF kappa B inhibitor, a nitric oxide agonist, an ornithine decarboxylase inhibitor, a p38 MAP kinase inhibitor, a palmitoyl-protein thioesterase inhibitor, a PDGF receptor kinase inhibitor, a peroxisome proliferator-activated receptor (PPAR) agonist, a phosphatase inhibitor, a phosphodiesterase inhibitor, a PKC inhibitor, a platelet activating factor antagonist, a prolyl hydroxylase inhibitor, a polymorphonuclear neutrophil inhibitor, protein kinase B inhibitor, protein kinase C stimulant, purine nucleoside analogue, a purineoreceptor P2X antagonist, a Raf kinase inhibitor, reversible inhibitor of ErbB1 and ErbB2, ribonucleoside triphosphate reductase inhibitor, an SDF-1 antagonist, a sheddase inhibitor, an SRC inhibitor, a stromelysin inhibitor, an Syk kinase inhibitor, a telomerase inhibitor, a TGF beta inhibitor, a TNF-alpha antagonist or TACE inhibitor, a tumor necrosis factor antagonist, a Toll receptor inhibitor, a tubulin antagonist, a protein tyrosine kinase inhibitor, a VEGF inhibitor, and a vitamin D receptor agonist.

73. The method of claim 67 or claim 68 wherein the anti-scarring agent is selected from a retinoic acid receptor antagonist, a heat shock protein 90 antagonist, a steroid, a cell cycle inhibitor, a histone deacetylase inhibitor, an anti-microbial agent, an intracellular calcium flux inhibitor, an microtubule inhibitor, an HMGCoA reductase inhibitor, an actin polymerization and stabilization promoter, a tyrosine kinase inhibitor, a TGF beta inhibitor, a TNF-alpha antagonist, a TACE inhibitor, a calcineurin inhibitor, a peptidyl-prolyl cis/trans isomerase inhibitor, an apoptosis activator, an antimetabolite and anti-neoplastic agent, a TGF beta inhibitor, a DNA methylation promoter, and a PKC inhibitor.

74. The method of claim 67 or claim 68 wherein the anti-scarring agent is selected from ZD-6474, AP-23573, synthadotin, S-0885, aplidine, ixabepilone, IDN-5390, SB-2723005, ABT-518, combretastatin, anecortave acetate, SB-715992, temsirolimus, adalimumab, erucylphosphocholine, alphastatin, etanercept, humicade, gefitinib, isotretinoin, radicicol, clobetasol propionate, homoharringtonine, trichostatin A, brefeldin A, thapsigargin, dolastatin 15, cerivastatin, jasplakinolide, herbimycin A, pirfenidone, vinorelbine, 17-DMAG, tacrolimus, loteprednol etabonate, juglone, prednisolone, puromycin, 3-BAABE, cladribine, mannose-6-phosphate, 5-azacytidine, Ly333531 (ruboxistaurin), and simvastatin.

75. The method of any one of claims 67-74 wherein the anti-infective agent is selected from an anthracycline, doxorubicin, mitoxantrone, fluoropyrimidine, 5-fluorouracil, a folic acid antagonist, methotrexate, podophylotoxin, etoposide, camptothecin, hydroxyurea, a platinum complex, and cisplatin.

76. The method of any one of claims 67-74 wherein the composition comprises an anti-thrombotic agent.

77. The method of any one of claims 67-74 wherein the polymer is formed from reactants comprising a naturally occurring polymer; protein; carbohydrate; biodegradable polymer; nonbiodegradable polymer; collagen; methylated collagen; fibrinogen; thrombin; blood plasma; calcium salt; an antifibronolytic agent; fibrinogen analog; albumin; plasminogen; von Willebrands factor; factor VIII; hypoallergenic collagen; atelopeptidic collagen; crosslinked collagen; aprotinin; epsilon-amino-n-caproic acid; gelatin; protein conjugates; gelatin conjugates; a synthetic polymer; isocyanate-containing compound; a synthetic thiol-containing compound; a synthetic compound containing at least two thiol groups; a synthetic compound containing at least three thiol groups; a synthetic compound containing at least four thiol groups; a synthetic amino-containing compound; a synthetic compound containing at least two amino groups; a synthetic compound containing at least three amino groups; a synthetic compound containing at least four amino groups; a synthetic compound comprising a carbonyl-oxygen-succinimidyl group; a synthetic compound comprising at least two carbonyl-oxygen-succinimidyl groups; a synthetic compound comprising at least three carbonyl-oxygen-succinimidyl groups; a synthetic compound comprising at least four carbonyl-oxygen-succinimidyl groups; a synthetic polyalkylene oxide-containing compound; a synthetic compound comprising both polyalkylene oxide and biodegradable polyester blocks; a synthetic polyalkylene oxide-containing compound having reactive amino groups; a synthetic polyalkylene oxide-containing compound having reactive thiol groups; a synthetic polyalkylene oxide-containing compound having reactive carbonyl-oxygen-succinimidyl groups; a synthetic compound comprising a biodegradable polyester block; a synthetic polymer formed in whole or part from lactic acid or lactide; a synthetic polymer formed in whole or part from glycolic acid or glycolide; polylysine; (a) protein and (b) a compound comprising a polyalkylene oxide portion; (a) protein and (b) polylysine; (a) protein and (b) a compound having at least four thiol groups; (a) protein and (b) a compound having at least four amino groups; (a) protein and (b) a compound having at least four carbonyl-oxygen-succinimide groups; (a) protein and (b) a compound having a biodegradable region formed from reactants selected from lactic acid, lactide, glycolic acid, glycolide, and epison-caprolactone; (a) collagen and (b) a compound comprising a polyalkylene oxide portion; (a) collagen and (b) polylysine; (a) collagen and (b) a compound having at least four thiol groups; (a) collagen and (b) a compound having at least four thiol groups; (a) collagen and (b) a compound having at least four amino groups; (a) collagen and (b) a compound having at least four carbonyl-oxygen-succinimide groups; (a) collagen and (b) a compound having a biodegradable region formed from reactants selected from lactic acid, lactide, glycolic acid, glycolide, and epison-caprolactone; (a) methylated collagen and (b) a compound comprising a polyalkylene oxide portion; (a) methylated collagen and (b) polylysine; (a) methylated collagen and (b) a compound having at least four thiol groups; (a) methylated collagen and (b) a compound having at least four amino groups; (a) methylated collagen and (b) a compound having at least four carbonyl-oxygen-succinimide groups; (a) methylated collagen and (b) a compound having a biodegradable region formed from reactants selected from lactic acid, lactide, glycolic acid, glycolide, and epison-caprolactone; hyaluronic acid; a hyaluronic acid derivative; pentaerythritol poly(ethylene glycol)ether tetra-sulfhydryl of number average molecular weight between 3,000 and 30,000; pentaerythritol poly(ethylene glycol)ether tetra-amino of number average molecular weight between 3,000 and 30,000; or (a) a synthetic compound having a number average molecular weight between 3,000 and 30,000 and comprising a polyalkylene oxide region and multiple nucleophilic groups, and (b) a synthetic compound having a number average molecular weight between 3,000 and 30,000 and comprising a polyalkylene oxide region and multiple electrophilic groups.
